(19) United States
(12) Patent Application Publication
(10) Pub. No.: US 2025/0236608 A1
(43) Pub. Date: Jul. 24, 2025

(54) EGFR INHIBITORS

(71) Applicant: Blueprint Medicines Corporation, Cambridge, MA (US)

(72) Inventors: Omar Ahmad, Cambridge, MA (US); Kevin K. Barvian, Cambridge, MA (US); John Emmerson Campbell, Cambridge, MA (US); Thomas A. Dineen, Cambridge, MA (US); Meredith Suzanne Eno, Cambridge, MA (US); Dilinie Prasadhini Fernando, Cambridge, MA (US); Emanuele Perola, Cambridge, MA (US); Vinicius Barros Ribeiro da Silva, Paris (FR); Quentin Perron, Paris (FR)

(21) Appl. No.: 18/853,933

(22) PCT Filed: Apr. 4, 2023
(86) PCT No.: PCT/US2023/017384

§ 371 Date: Oct. 3, 2024

Related U.S. Application Data
(60) Provisional application No. 63/327,631, filed on Apr. 5, 2022.

(51) Int. Cl. C07D 401/14; A61K 31/517; A61K 31/519; A61K 31/5377; A61K 31/538; A61K 31/5386; C07D 403/04; C07D 403/14; C07D 405/14; C07D 413/14; C07D 417/04; C07D 417/14; C07D 471/04; C07D 487/04; C07D 487/10; C07D 491/107; C07D 498/08; C07D 519/00

(52) U.S. Cl. CPC C07D 401/14; A61K 31/517; A61K 31/519; A61K 31/5377; A61K 31/538; A61K 31/5386; C07D 403/04; C07D 403/14; C07D 405/14; C07D 413/14; C07D 417/04; C07D 417/14; C07D 471/04; C07D 487/04; C07D 487/10; C07D 491/107; C07D 498/08; C07D 519/00

(57) ABSTRACT

The present disclosure provides a compound represented by structural formula (A) or (I): or a pharmaceutically acceptable salt thereof useful for treating a cancer.

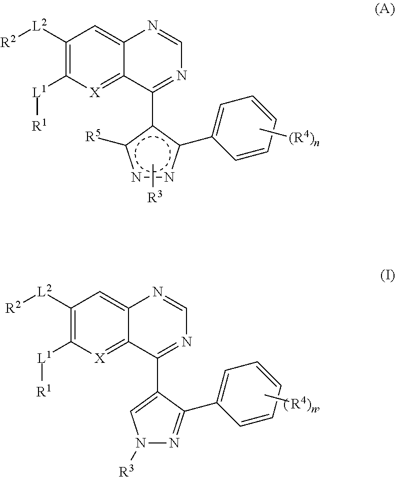

### CROSS-REFERENCE TO RELATED APPLICATIONS

This application claims priority from U.S. Provisional Application No. 63/327,631, filed Apr. 5, 2022. The entire contents of the aforementioned application are incorporated herein by reference.

### BACKGROUND

EGFR (Epidermal Growth Factor Receptor) is a member of the erbB receptor family, which includes transmembrane protein tyrosine kinase receptors. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors. EGFR gene amplification and alterations such as mutations, deletions and the like, can promote malignant transformation of cells and play an important role in tumor cell proliferation, invasion, metastasis and angiogenesis. For example, alterations such as mutations and deletions in the EGFR gene are found in non-small lung cancer (NSCLC) tumors. The two most frequent EGFR alternations found in NSCLC tumors are short in-frame deletions in exon 19 (del19) and L858R, a single missense mutation in exon 21 (Cancer Discovery 2016 6 (6) 601). These two alterations, referred to as sensitizing mutations, cause ligand-independent EGFR activation and are referred to as primary or activating mutations in EGFR mutant NSCLC (EGFR M+). Clinical experience shows an objective response rate (ORR) of approximately 60-85% in EGFR M+ NSCLC patients treated first line (1 L) with EGFR tyrosine kinase inhibitors (TKIs) erlotinib, gefitinib, afatinib and osimertinib (Lancet Oncol. 2010 Vol. 11, 121; Lancet Oncol. 2016 Vol. 17, 577; N. Engl. J. Med. 2017 Nov. 18 Doi: 10.1056/NEJMoal713137; Lancet Oncol. 2011 Vol. 12, 735), thus demonstrating that EGFR mutant NSCLC tumors depend on oncogenic EGFR activity for survival and proliferation and establishing del 19 and L858R mutated EGFR as oncogenic drivers of disease and thus, validating drug targets and biomarkers for the treatment of NSCLC.

Osimertinib is a covalent third (3^{rd}) generation EGFR TKI that is now the approved standard of care (SOC) in first line (1 L) for the treatment of NSCLC harboring del 19 and L858R mutations. With a progression-free survival (PFS) of 18.9 mo (J C Soria et al—NEJM, 2018 January: 378 (2): 113-125), it shows a transformative outcome for patients compared to first generation TKIs. However, after an average of 10-12 months of treatment, resistance has been observed in almost all NSCLC patients (Lancet Oncol. 2010 February: 11(2): 121-8: Lancet Oncol. 2016 May: 17 (5): 577-89; Lancet Oncol. 2011 August: 12 (8): 735-42). Additional 3rd generation TKIs are being used in front line (e.g. lazertinib) and relay on the same covalent mechanism of binding to EGFR. The most prominent on-target resistance mechanism is due to the secondary mutation in EGFR of C797X (where “X” can be an “S” or a “G” or an “N” or a “Y” or a “T” or a “D”), which occurs in 7% to 22% of patients progressing on 3rd generation EGFR inhibitors used in front line (Blakely, 2012; Kobayashi, 2005). This secondary C797S mutation reduces the affinity of the drug with the target, thereby producing drug resistance, and resulting in tumor recurrence or disease progression. The resulting “double mutant” tumors, that harbors the sensitizing mutations del19 or L858R and the resistance mutation C797X (e.g., C797S), are no longer sensitive to 2^{nd} and 3^{rd} generation TKIs. There is no approved drug to treat the double mutant patients. 1^{st} generation TKIs (gefitinib and erlotinib) are active against C797X (e.g., C797S) but they are poorly tolerated due to activity associated with wild-type EGFR inhibition, and do not control brain disease due to their low ability to cross the blood brain barrier (BBB).

There is an unmet need for a selective therapeutic agent that treats the double mutant tumors, that is brain penetrant and treats the brain disease, and with reduced toxicologies (diarrhea, skin rash) associated with wild-type EGFR inhibition.

### SUMMARY

The applicant has discovered novel compounds which are effective inhibitors of certain mutant forms of EGFR (see Synthetic Examples 1-454 and 458-500). In particular, it has been demonstrated that the compounds of the present disclosure effectively inhibit certain mutant forms of EGFR. Compounds of the disclosure (also referred to herein as the “disclosed compounds”) or pharmaceutically acceptable salts thereof effectively inhibit EGFR with one or more alterations, including L858R or exon 19 deletion mutation, and C797X (e.g., C797S) mutation (hereinafter “EGFR with LRCS mutations” or “double mutant EGFR”) (see Biological Example 1) and can be used treat various cancers, for example, lung cancer (see Biological Example 2). Importantly, the disclosed compounds are selective EGFR inhibitors, i.e., the disclosed compounds have no or low activity against wild-type EGFR and the kinome. Advantages associated with such selectivity may include facilitating efficacious dosing and reducing EGFR-mediated on-target toxicities. Some of the disclosed compounds exhibit good penetration of the brain and blood brain barrier (e.g., a PGP efflux ratio of less than 5). As such, the compounds of the disclosure or pharmaceutically acceptable salts thereof are expected to be effective for the treatment of metastatic cancer, including brain metastesis, including leptomeningeal disease and other systemic metastesis. Some of the disclosed compounds also have the advantage of having high microsomal stability. Compounds of the disclosure also may have favorable toxicity profiles related to other non-kinase targets.

In one aspect, the present disclosure provides a compound represented by the following structural Formula (A) or (I):

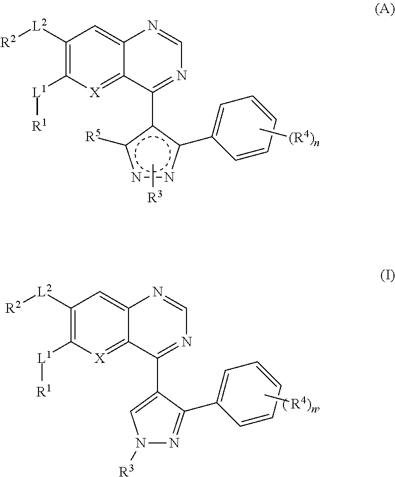

or a pharmaceutically acceptable salt thereof, the definition of each variable is provided below.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (A) or (I)) or a pharmaceutically acceptable salt thereof or a pharmaceutical composition of the disclosure. In one embodiment, the cancer is non-small cell lung cancer. In another embodiment, the subject cancer has metastasized to the brain. In another embodiment, the subject has brain metastasis from non-small cell lung cancer.

In one embodiment, the cancer to be treated has epidermal growth factor receptor (EGFR) L858R mutation or exon 19 deletion mutation. In another embodiment, the cancer to be treated may further has epidermal growth factor receptor (EGFR) L858R mutation or exon 19 deletion mutation and the C797X (e.g., C797S) mutation. In another embodiment, the cancer to be treated in either of the foregoing embodiments is lung cancer, e.g., non-small cell lung cancer. In a specific embodiment, the cancer is non-small cell lung cancer with brain metastasis or leptomeningeal disease.

The treatment method disclosed herein further comprises administering to the subject an effective amount of an EGFR inhibitor (e.g., afatinib and/or osimertinib), and a MET inhibitor in combination with an effective amount of a compound of the disclosure.

The present disclosure also provides a method of inhibiting epidermal growth factor receptor (EGFR) in a subject in need thereof, comprising administering to the subject an effective amount of a compound of the disclosure (e.g., a compound of Formula (A) or (I)) or a pharmaceutically acceptable salt thereof or a pharmaceutical composition of the disclosure.

The present disclosure also provides the use of an effective amount of a compound of the disclosure (e.g., a compound of Formula (A) or (I)), or a pharmaceutically acceptable salt thereof, or a pharmaceutical composition of the disclosure, for the preparation of a medicament for the treatment of cancers.

In another aspect, provided herein a compound of Formula (A) or (I), or a pharmaceutically acceptable salt thereof, or a pharmaceutical composition of the disclosure for use in treating cancers.

### DETAILED DESCRIPTION

### Definitions

The term “halo” as used herein means halogen and includes chloro, fluoro, bromo and iodo.

The term “alkyl” used alone or as part of a larger moiety, such as “alkoxy” and the like, means saturated aliphatic straight-chain or branched monovalent hydrocarbon radical. Unless otherwise specified, an alkyl group typically has 1-6 carbon atoms, i.e. (C₁-C₆)alkyl. As used herein, a “(C₁-C₆)alkyl” group means a radical having from 1 to 6 carbon atoms in a linear or branched arrangement. Examples include methyl, ethyl, n-propyl, iso-propyl, and the like.

The term “haloalkyl” or “C₁₋₄haloalkyl” refers to an alkyl group wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “cycloalkyl” refers to a monocyclic or bicyclic or polycyclic saturated hydrocarbon ring system. Cycloalkyl may include fused and/or bridged rings and/or spirocyclic rings. Non-limiting examples of fused/bridged cycloalkyl include: bicyclo[1.1.0]butane, bicyclo[2.1.0]pentane, bicyclo[1.1.0]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2,2,2]octane, and the like. Cycloalkyl also includes spirocyclic rings (e.g., spirocyclic bicycle wherein two rings share one ring atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. Unless otherwise specified, cycloalkyl has from 3-12 carbon atoms. For example, a C₃-C₆cycloalkyl includes cyclopropyl, cyclobutyl, cyclopentyl and cyclohexyl. Unless otherwise described, a “cycloalkyl” has from three to six carbon atoms.

The term “heterocyclyl” or “heterocyclic” refers to a radical of a 4- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“4-12 membered heterocyclyl”). In some embodiments, a heterocyclyl group is a 4- to 8-membered non-aromatic ring system having ring carbon atoms and 1-4 (typically 1 to 2) ring heteroatoms, wherein each heteroatom is independently selected from nitrogen, oxygen, and sulfur (“4-8 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or a tricyclic system (“tricyclic heterocyclyl”)). A polycyclic ring system includes fused, bridged, or spiro ring systems. When a heterocyclyl group is a polycyclic ring system, said ring system includes at least one non-aromatic ring. Exemplary monocyclic heterocyclyl groups include azetidinyl, oxetanyl, thietanyl, tetrahydrofuranyl, pyrrolidinyl, pyrrolidin-2-onyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system-including polycyclic ring systems having a non-aromatic ring fused to a phenyl or heteroaryl ring. Exemplary polycyclic heterocyclic groups include 2H-benzo[b][1,4]oxazin-3 (4H)-onyl, isoindolin-1-onyl, isoquinolin-1(2H)-onyl, 3-oxabicyclo[3.1.0]hexanyl, 8-oxa-3-azabicyclo[3.2.1]octanyl, 2-oxa-6-azaspiro[ 3.3]heptanyl, 6-oxa-3-azabicyclo[3.1.1]heptanyl, tetrahydropyrazolo[1,5-a]pyridinyl, and the like. Substituents may be present on one or more rings in the polycyclic ring system.

“Heteroaryl” refers to a radical of a 4- to 12-membered aromatic ring system having ring carbon atoms and 1 to 4 ring heteroatoms, wherein each heteroatom is independently selected from nitrogen, oxygen, and sulfur. In some embodiments, a heteroaryl group is a 5 or 6 membered heteroaryl having ring carbon atoms and 1 to 4 ring heteroatoms (typically 1 to 2). Representative heteroaryl groups include ring systems where each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl.

A bridged bicyclic system has two non-aromatic rings containing from 5-12 ring atoms (heterocyclyl or cycloalkyl) and which share three or more ring atoms, with the two bridgehead ring atoms separated by a bridge containing at least one atom. “Bridged heterocyclyl” includes bicyclic or polycyclic hydrocarbon or aza-bridged hydrocarbon groups: examples include bicyclo[1.1.1]pentanyl, 3-oxabicyclo[3.1.0]hexanyl, 2-azabicyclo[2.2.1]heptanyl, 6-oxa-3-azabicyclo[3.1.1]heptanyl, 3-azabicyclo[3.2.1]octanyl, 6-oxa-2-azabicyclo[3.2.1]octanyl, 6-oxa-3-azabicyclo[3.2.1]octanyl, and 8-oxa-3-azabicyclo[3.2.1]octanyl.

A fused bicyclic system has two rings containing from 6-12 ring atoms and which share two adjacent ring atoms. When the fused bicyclic system is heterocyclyl, at least one of the rings is non-aromatic. Examples of fused bicyclic systems include hexahydro-1H-furo[3,4-b]pyrrolyl, and hexahydro-1H-furo[3,4-c]pyrrolyl.

A spiro bicyclic system has two non-aromatic rings containing (heterocyclyl or cycloalkyl) from 7-12 ring atoms and which share one ring atom. Examples of spiro bicyclic systems include 1-oxa-7-azaspiro[3.5]nonan-7-yl, 2-oxa-6-azaspiro[3.3]heptanyl, 1,4-dioxa-8-azaspiro[4.5]decan-8-yl, and 1,4-dioxa-9-azaspiro[5.5]undecan-9-yl.

### Compounds of the Present Disclosure

Disclosed herein are embodiments of compounds having a general structure of Formula (A) or (I). These compounds are selective inhibitors of L858R, Ex19del, L858RC797S and Ex19DelC797S EGFR. In contrast to other EGFR inhibitors such as osimertinib which binds EGFR irreversibly, the compounds of the disclosure are non-covalent inhibitors.

In a first embodiment, the present disclosure provides a compound represented by the following structural formula (A):

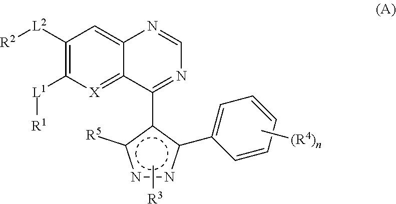

or a pharmaceutically acceptable salt thereof, wherein
   X is CR^{x} or N;
   R^{x} is H or F;
   L¹ is a bond, NH, —NHC(O)—*, —NHC(O)O—*, O, or —OC(O)—*; wherein -* represents the point which attaches to R¹;
   L² is a bond or O;
   R¹ is H; or
      C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl; or
      C₃-C₈cycloalkyl, phenyl, 4 to 12 membered heterocyclyl, or 5 to 12 membered heteroaryl, wherein the cycloalkyl, phenyl, heterocyclyl and heteroaryl represented by R¹ are each optionally substituted with 1 to 4 groups independently selected from R¹¹;
   each R¹¹ is independently selected from halo, deuterium, OR^{a}, C(O)R^{a}, C(O)NR^{a}R^{b}, NR^{a}C(O)OR^{a}, NR^{a}R^{b}, S(O)₂R^{a}, C₁-C₄alkyl, C₃-C₆cycloalkyl, phenyl, 4 to 12 membered heterocyclyl and 5 to 12 membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄alkyl, OR^{a} and NR^{a}R^{b}, or two R¹¹ which are attached to the same carbon atom are taken together to form ═O;
   R² is:
      H, C₁-C₄alkyl, C₃-C₈cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl; or
      4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl;
   R³ is attached to either nitrogen atom in the pyrazole ring, and is selected from H, deuterium, C₁-C₄alkyl, C₃-C₆cycloalkyl, and 4 to 6 membered heterocyclyl, wherein the alkyl, cycloalkyl and heterocyclyl represented by R³ are each optionally substituted with 1 to 4 groups independently selected from halo, deuterium, OR^{a}, NR^{a}R^{b} and C₃-C₆cycloalkyl;
   each R⁴ is independently selected from halo, deuterium and OR^{a}:
   R⁵ is selected from H, deuterium, halo and C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl;
   each R^{a} is independently selected from H, deuterium, C₁-C₄alkyl, C₁-C₄haloalkyl, C₃-C₆cycloalkyl and 4 to 6 membered heterocyclyl;
   each R^{b} is independently selected from H, deuterium and C₁-C₄alkyl; and
   n is 0, 1, 2, 3, 4 or 5.

In a second embodiment, the compound according to structural formula (A) is represented by a structural formula selected from (B) and (C):

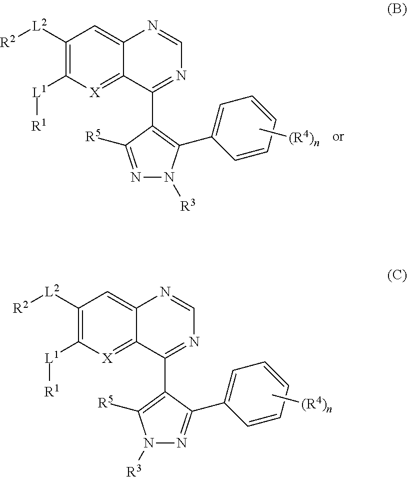

or a pharmaceutically acceptable salt thereof, wherein the variables are as defined in the first embodiment.

In a third embodiment, the present disclosure provides a compound according to structural formulas (A), (B) or (C), or a pharmaceutically acceptable salt thereof, wherein when L¹ and L² are each a bond, R¹ is H, R² is not H, and wherein the remainder of the variables are as defined in the first embodiment.

In a fourth embodiment, the present disclosure provides a compound according to structural formulas (A), (B) or (C), or a pharmaceutically acceptable salt thereof, wherein R² is C₁-C₄alkyl, C₃-C₈cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl; or 4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl, and wherein the remainder of the variables are as defined in the first or third embodiment.

In a fifth embodiment, the present disclosure provides a compound according to structural formula (A), (B) or (C), or a pharmaceutically acceptable salt thereof, wherein R⁵ is H, F or methyl, and wherein the remainder of the variables are as defined in the first, third or fourth embodiment.

In a sixth embodiment, the present disclosure provides a compound represented by the following structural formula (I):

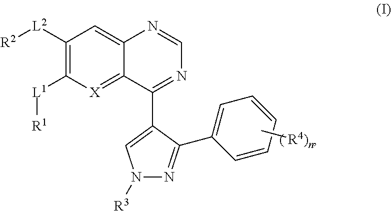

or a pharmaceutically acceptable salt thereof, wherein
   X is CR^{x} or N;
   R^{x} is H or F;
   L¹ is a bond, NH, —NHC(O)—*, —NHC(O)O—*, O, or —OC(O)—*; wherein -* represents the point which attaches to R¹;
   L² is a bond or O;
   R¹ is H; or
      C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl; or
      C₃-C₈cycloalkyl, phenyl, 4 to 12 membered heterocyclyl, or 5 to 12 membered heteroaryl, wherein the cycloalkyl, phenyl, heterocyclyl and heteroaryl represented by R¹ are each optionally substituted with 1 to 4 groups independently selected from R¹¹;
   each R¹¹ is independently selected from halo, deuterium, OR^{a}, C(O)R^{a}, C(O)NR^{a}R^{b}, NR^{a}C(O)OR^{a}, NR^{a}R^{b}, S(O)₂R³, C₁-C₄alkyl, C₃-C₆cycloalkyl, phenyl, 4 to 12 membered heterocyclyl and 5 to 12 membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄alkyl, OR^{a} and NR^{a}R^{b}, or two R¹¹ which are attached to the same carbon atom are taken together to form ═O;
   R² is:
      C₁-C₄alkyl, C₃-C₈cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl; or
      4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl;
   R³ is H, deuterium, C₁-C₄alkyl, C₃-C₆cycloalkyl or 4 to 6 membered heterocyclyl, wherein the alkyl, cycloalkyl and heterocyclyl represented by R³ are each optionally substituted with 1 to 4 groups independently selected from halo, deuterium, OR^{a}, NR^{a}R^{b} and C₃-C₆cycloalkyl;
   each R⁴ is independently selected from halo, deuterium and OR^{a};
   each R^{a} is independently selected from H, deuterium, C₁-C₄alkyl, C₁-C₄haloalkyl, C₃-C₆cycloalkyl and 4 to 6 membered heterocyclyl;
   each R^{b} is independently selected from H, deuterium and C₁-C₄alkyl; and
   n is 0, 1, 2, 3, 4 or 5. Alternatively, the variables in Structural Formula (I) are as defined in the first, third or fourth embodiment.

In a seventh embodiment, the compound according to structural formula (I) is represented by one of the structural formulas (II-1), (II-2), (II-3), (II-4), (II-5), (II-6), (II-7), (II-8), (II-9), (II-10), (II-11) or (II-12):

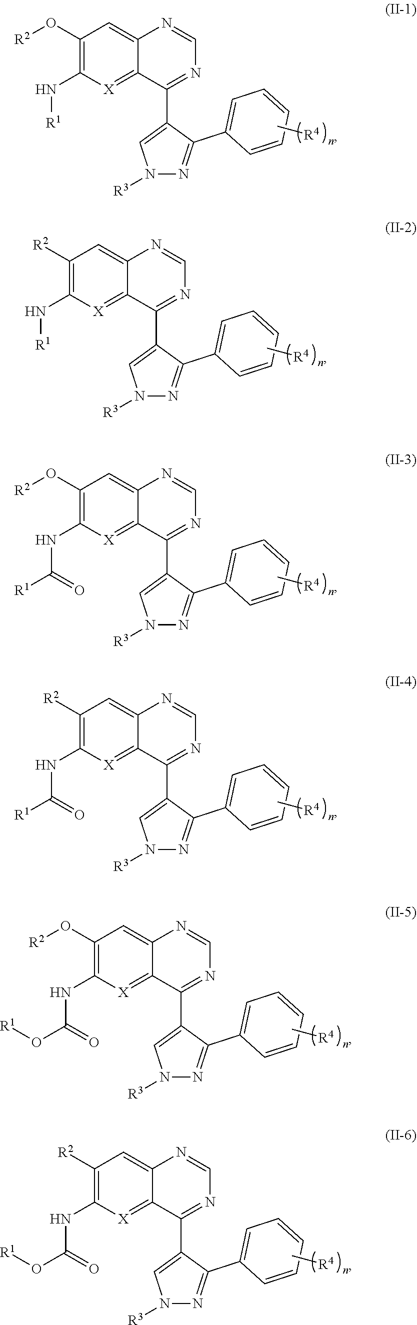

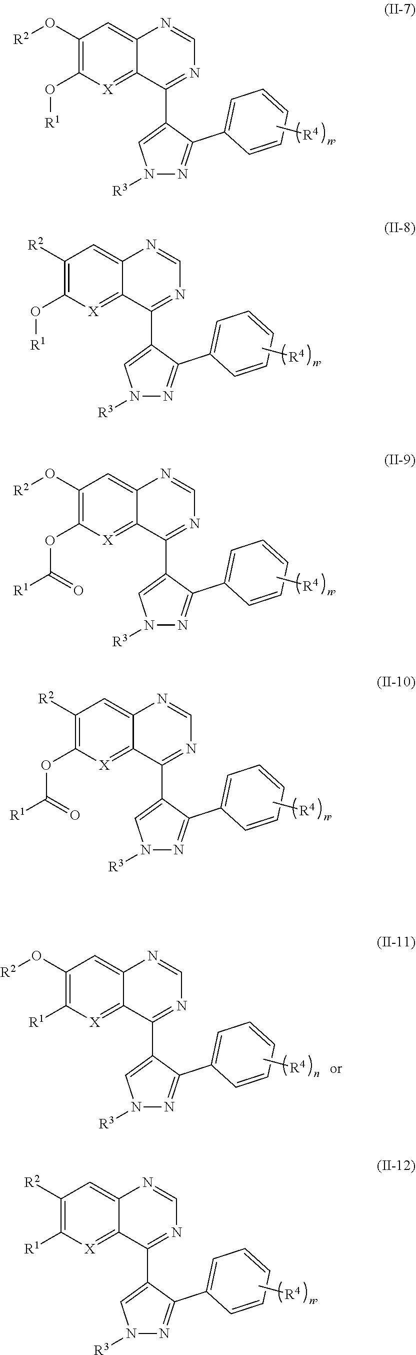

or a pharmaceutically acceptable salt thereof, wherein the variables are as defined in the first, third, fourth, or sixth embodiment.

In a eighth embodiment, the compound of formula (I) is represented by one of structural formulas (II-1a), (II-3a), (II-3b), (II-4a), (II-5a), (II-7a), (II-7b), (II-8a), (II-9a), (II-9b), (II-11a), (II-11b) or (II-12a):

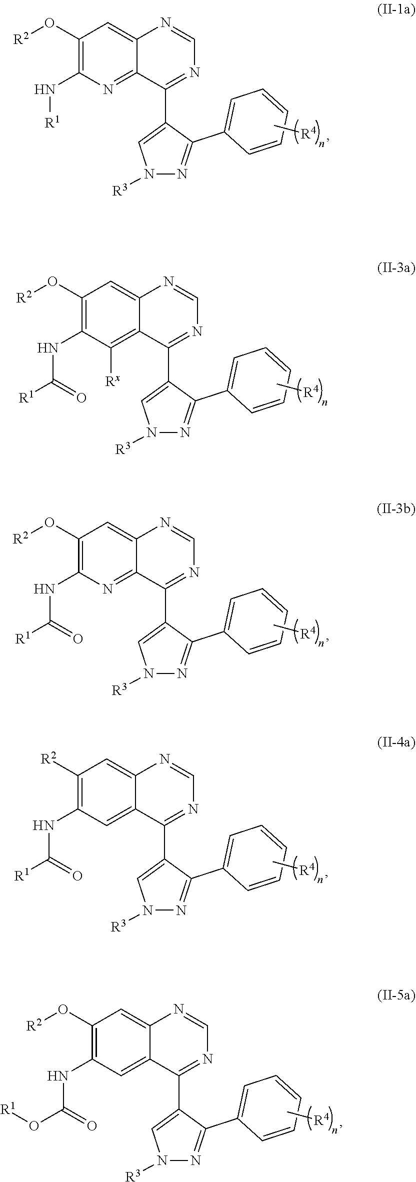

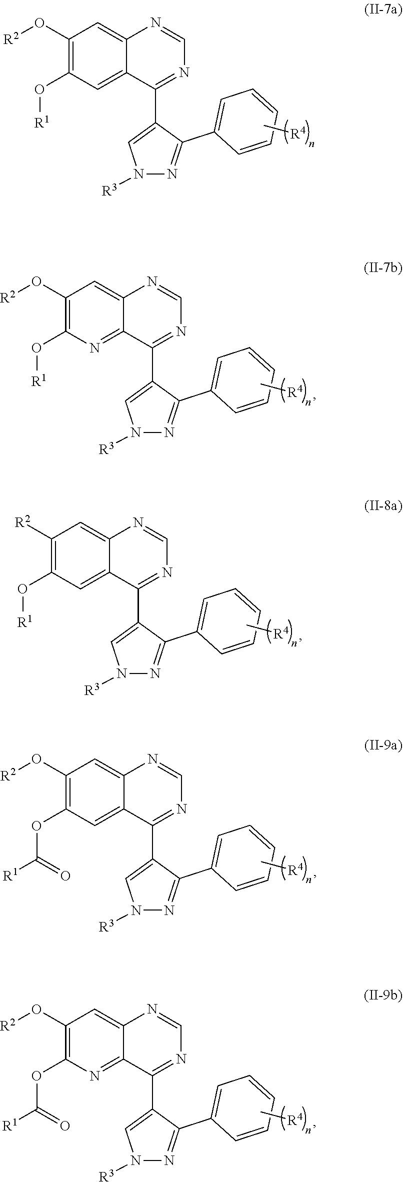

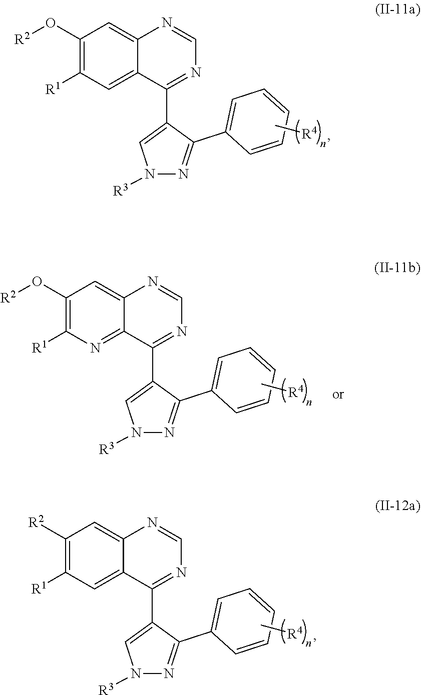

or a pharmaceutically acceptable salt thereof, wherein the variables are as defined in the first, third, fourth, or sixth embodiment.

In a ninth embodiment, the present disclosure provides a compound according to structural formulas (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R^{x} is H, and wherein the remainder of the variables are as defined in the first, third, fourth, fifth, or sixth embodiment.

In a tenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R¹ is H; or C₁-C₃alkyl optionally substituted with 1 to 2 groups independently selected from halo, OR^{a}, NR^{a}R^{b}, C₃-C₅cycloalkyl, 4 to 6 membered heterocyclyl and 5 to 6 membered heteroaryl, wherein the heterocyclyl and heteroaryl are each optionally substituted with 1 to 2 groups independently selected from halo and —CH₃; or C₃-C₈cycloalkyl, phenyl, 4 to 9 membered heterocyclyl or 5 to 10 membered heteroaryl, wherein the cycloalkyl, phenyl, heterocyclyl and heteroaryl in the group represented by R¹ are each optionally substituted with 1 to 3 groups independently selected from R¹¹; and each R^{a} is independently H or —CH₃; and each R^{b} is —CH₃, and wherein the remainder of the variables are as defined in the first, third, fourth, fifth, sixth, or ninth embodiment.

In an eleventh embodiment, the present disclosure provides a compound according to structural formulas (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R¹ is H; or R¹ is selected from —CH₃, —CH₂CH₃, —CH₂CH₃CH₃ and —CH(CH₃)₂, each of which is optionally substituted with 1-3 groups selected from F, —OH, —OCH₃, —N(CH₃)₂, cyclopropyl, morpholinyl, oxadiazolyl, oxetanyl, oxazolyl, pyridinyl, tetrahydrofuranyl, tetrazolyl, thiazolyl and triazolyl, wherein the pyridinyl, thiadiazolyl, thiazolyl and triazolyl are each optionally substituted with F, —CH₃ or —CH₂CH₃; or R¹ is selected from azabicyclo[3.1.0]hexanyl, azetidinyl, 1H-benzo[d]imidazolyl, bicyclo[1.1.1]pentanyl, cubanyl, cyclobutyl, cyclopropyl, dihydroisoquinolinyl, dihydropyrrolyl, dihydro-2H-benzo[b][1,4]oxazinyl, dioxepanyl, hexahydro-1H-pyrrolizinyl, imidazolidinyl, indazolyl, isoindolinyl, isothiazolyl, morpholinyl, octahydropyrrolo[1,2-a]pyrazinyl, oxabicyclo[3.1.0]hexanyl, 6-oxa-3-azabicyclo[3.1.1]heptanyl, oxabicyclo[3.3]heptanyl, 7-oxa-4-azaspiro[2.5]octanyl, oxetanyl, phenyl, piperidinyl, pyrazolyl, pyridazinyl, pyridinyl, pyrimidinyl, pyrrolidinyl, pyrrolopyridinyl, tetrahydrofuranyl, tetrahydropyranyl and thiazolyl, each of which is optionally substituted with 1 to 3 groups independently selected from R¹¹, and wherein the remainder of the variables are as defined in the first, third, fourth, fifth, sixth, ninth or tenth embodiment.

In a twelfth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R¹ is selected from H, —CH₃, —CH₂(R¹¹), —CH(R¹¹)₂, —CH₂CH₃, —CH(R¹¹)—CH₃, —CH(CH₃)₂, —C(CH₃)₂—R¹¹, —CH₂CH₂CH₂—R¹¹, —CH(CH₃)CH₂—R¹¹,

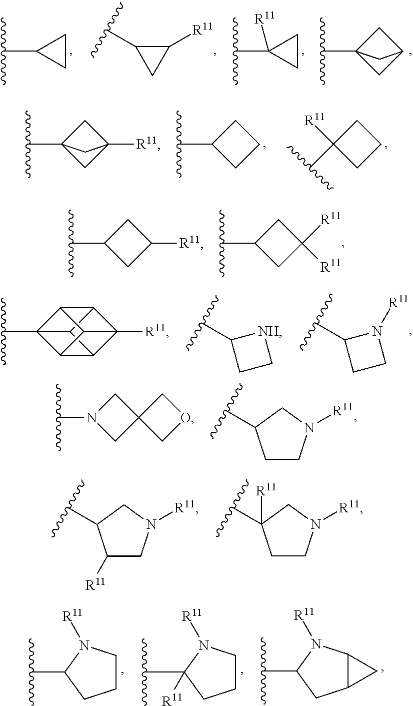

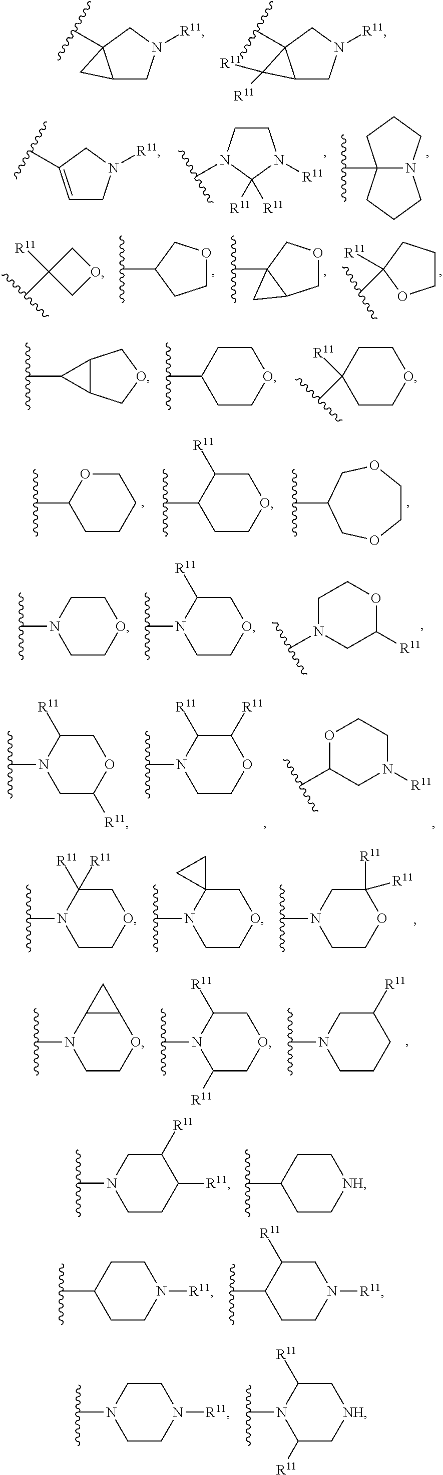

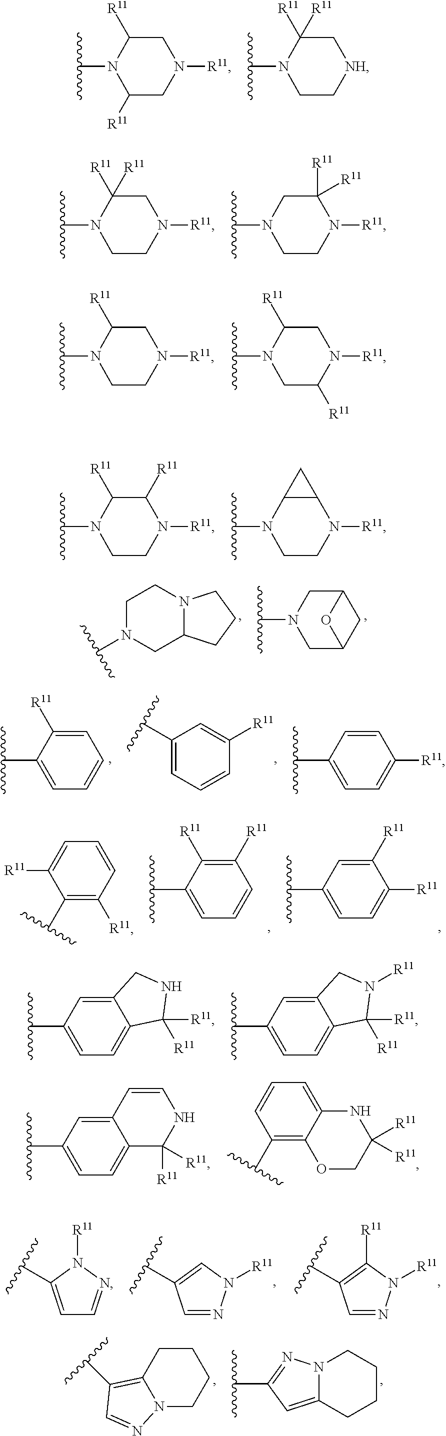

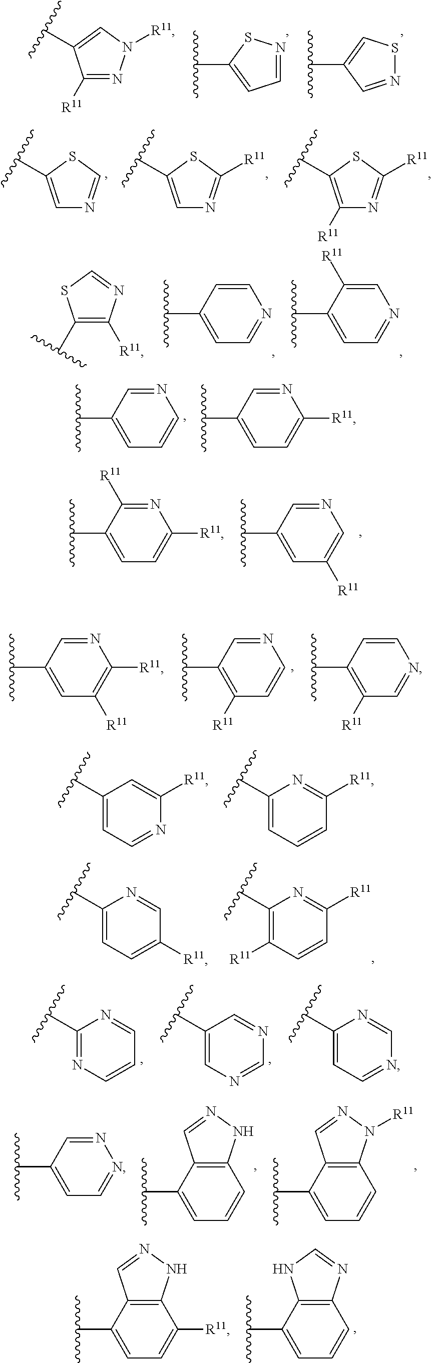

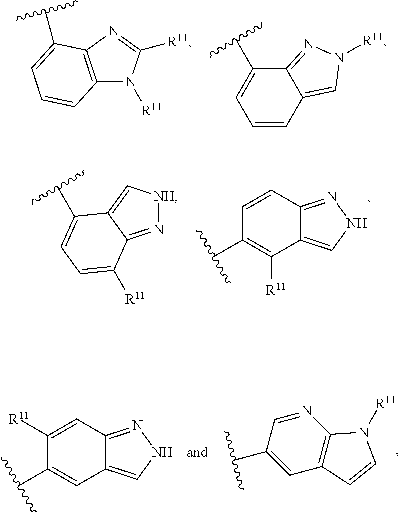

and wherein the remainder of the variables are as defined in the first, third, fourth, fifth, sixth, ninth, tenth or eleventh embodiments.

In a thirteenth embodiment, the present disclosure provides a compound according to structural formula (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein each R¹¹ is independently selected from halo, OR^{a}, C(O)R^{a}, C(O)NR^{a}R^{b}, NR^{a}C(O)OR^{a}, NR^{a}R^{b}, C₁-C₄alkyl, C₃-C₆cycloalkyl, 4 to 12 membered heterocyclyl, and 5 to 12 membered heteroaryl, wherein the alkyl, cycloalkyl, heterocyclyl, and heteroaryl represented by R¹¹ are each optionally substituted with 1 to 3 groups selected from deuterium, halo, OR^{a} and NR^{a}R^{b}, or two R¹¹ which are attached to the same carbon atom are taken together to form ═O; each R^{a} is independently selected from H, C₁-C₄alkyl, C₃-C₆cycloalkyl, and 4 to 6 membered heterocyclyl; and each R^{b} is independently selected from H and C₁-C₄alkyl, and wherein the remainder of the variables are as defined in the first, third, fourth, fifth, sixth, ninth, tenth, eleventh or twelfth embodiments.

In a fourteenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein each R¹¹ is independently selected from halo, OR^{a}, C(O)R^{a}, C(O)NR^{a}R^{b}, NR^{a}C(O)OR^{a}, NR^{a}R^{b}, C₁-C₃alkyl, morpholinyl, oxazolyl, oxadiazolyl, oxetanyl, pyridinyl, tetrahydrofuranyl, tetrazolyl, thiadiazolyl and thiazolyl, wherein the alkyl, morpholinyl, oxazolyl, oxadiazolyl, oxetanyl, pyridinyl, tetrahydrofuranyl, tetrazolyl, thiadiazolyl and thiazolyl represented by R¹¹ are each optionally substituted with 1 to 3 groups selected from deuterium, halo, OR^{a} and NR^{a}R^{b}, or two R¹¹ which are attached to the same carbon atom are taken together to form ═O; each R^{a} is independently selected from H, —CH₃, —CH₂CH₃, —C(CH₃)₃,

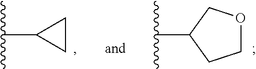

and each R^{b} is independently H or —CH₃, and wherein the remainder of the variables are as defined in first, third, fourth, fifth, sixth, ninth, tenth, eleventh, twelfth or thirteenth embodiment.

In a fifteenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein each R¹¹ is independently selected from Cl, F, —OH, —OCH₃, —C(O)CH₂CH₃, —N(CH₃)₂, —NHC(O)OC(CH₃)₃, —CH₃, —CD₃, —CHF₂, —CF₃, —CH₂OH, —CH₂OCH₃, —CH₂—N(CH₃)₂, —CH₂CH₃, —CH₂CF₃, —CH₂CH₂—N(CH₃)₂, —CH(CH₃)₂, —C(CH₃)₂—OH,

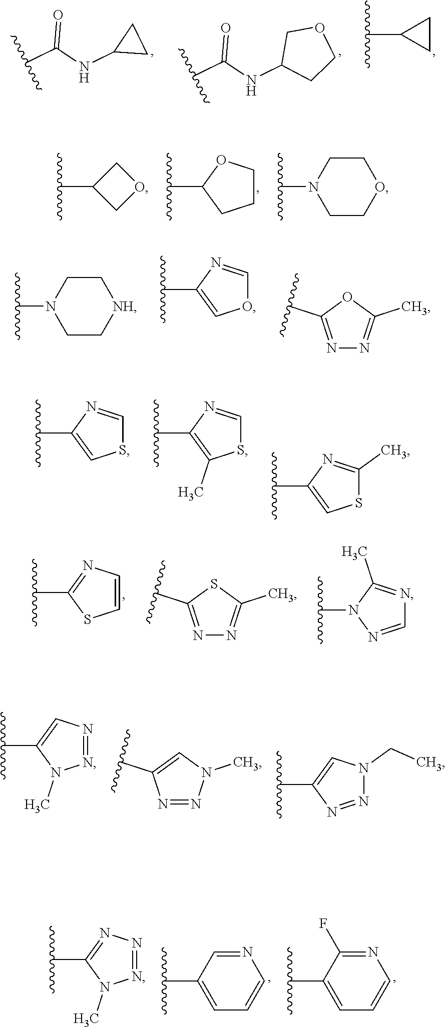

or two R¹¹ which are attached to the same carbon atom are taken together to form ═O, and wherein the remainder of the variables are as defined in the first, third, fourth, fifth, sixth, ninth, tenth, eleventh, twelfth, thirteenth or fourteenth embodiment.

In a sixteenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein L² is a O, and wherein the remainder of the variables are as defined in the first, third, fourth, fifth, sixth, ninth, tenth, eleventh, twelfth, thirteenth, fourteenth or fifteenth embodiment.

In a seventeenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R² is C₁-C₄alkyl, C₃-C₅cycloalkyl, C₂-C₄alkenyl or C₂-C₄alkynyl, each of which is optionally substituted with 1 to 3 groups independently selected from halo, OR^{a}, NR^{a}R^{b} and 4 to 12 membered heterocyclyl, or 4 to 12 membered heterocyclyl or 5 to 12 membered heteroaryl, wherein the heterocyclyl and heteroaryl represented by R² are each optionally substituted with 1 to 4 groups selected from C₁-C₄alkyl, C(O)R^{a} and 4 to 12 membered heterocyclyl which is optionally substituted with 1 to 3 groups selected from halo, and C₁-C₄alkyl; each R^{a} is independently selected from H and C₁-C₄alkyl; and each R^{b} is independently selected from H and C₁-C₄alkyl, and wherein the remainder of the variables are as defined in the first, third, fourth, fifth, sixth, ninth, tenth, eleventh, twelfth, thirteenth, fourteenth, fifteenth or sixteenth embodiment.

In an eighteenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R² is C₁-C₄alkyl, C₃-C₅cycloalkyl or C₃-C₄alkynyl, each of which is optionally substituted with 1 to 2 groups independently selected from halo, OR^{a}, NR^{a}R^{b} and 4 to 6 membered heterocyclyl, or 5 to 7 membered heterocyclyl or 5 to 6 membered heteroaryl, wherein the heterocyclyl and heteroaryl in the group represented by R² are each optionally substituted with 1 to 2 groups selected from C₁-C₂alkyl, C(O)R^{a} and 6 membered heterocyclyl optionally substituted with C₁-C₂alkyl; each R^{a} is independently selected from H and —CH₃; and each R^{b} is —CH₃, and wherein the remainder of the variables are as defined in the first, third, fourth, fifth, sixth, ninth, tenth, eleventh, twelfth, thirteenth, fourteenth, fifteenth, sixteenth or seventeenth embodiment.

In a nineteenth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R² is —CH₃, —CH₂CH₃, —CH(CH₃)₂, cyclopropyl or

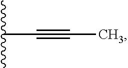 [SMILES: *C#CC]

each of which is optionally substituted with F, —OCH₃, —N(CH₃)₂, morpholinyl or oxetanyl; or R² is diazaspiro[3.3]heptanyl, pyrazolyl, pyrimidinyl or tetrahydrofuranyl, each of which is optionally substituted with C₁-C₂alkyl, C(O)C₁-C₄alkyl or piperidinyl optionally substituted with —CH₃, and wherein the remainder of the variables are as defined in the first, third, fourth, fifth, sixth, ninth, tenth, eleventh, twelfth, thirteenth, fourteenth, fifteenth, sixteenth, seventeenth or eighteenth embodiment.

In a twentieth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R² is —CH₃, —CHF₂, —CH₂CH₃, —CH₂CH₂—OCH₃, —CH₂CH₂—N(CH₃)₂, —CH(CH₃)₂, cyclopropyl,

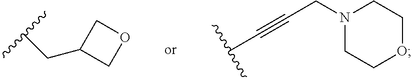 [SMILES: *CC1COC1 ; "or" ; *C#CCN1CCOCC1]

### or R² is

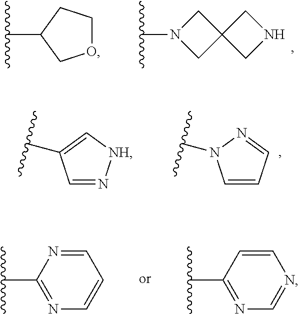

each of which is optionally substituted with —CH₃, C(O)CH₃ or

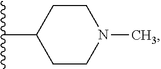 [SMILES: *C1CCN(C)CC1]

and wherein the remainder of the variables are as defined in the first, third, fourth, fifth, sixth, ninth, tenth, eleventh, twelfth, thirteenth, fourteenth, fifteenth, sixteenth, seventeenth, eighteenth or nineteenth embodiment.

In a twenty-first embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R³ is H, C₁-C₄alkyl, C₃-C₆cycloalkyl or 4 to 6 membered heterocyclyl, wherein the alkyl, cycloalkyl and heterocyclyl represented by R³ are each optionally substituted with 1 to 3 groups independently selected from halo, deuterium, OR^{a}, NR^{a}R^{b} and C₃-C₆cycloalkyl; each R^{a} is independently H or C₁-C₄alkyl; and each R^{b} is independently H or C₁-C₄alkyl, and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth or twentieth embodiment.

In a twenty-second embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R³ is H, cyclopropyl, oxetanyl, tetrahydropyanyl or C₁-C₃alkyl optionally substituted with 1 to 3 groups independently selected from halo, deuterium, OH, N(CH₃)₂ and cyclopropyl, and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth, twentieth or twenty-first embodiment.

In a twenty-third embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein R³ is selected from H, —CH₃, —CHF₂, —CD₃, —CH₂CH₃, —CH₂CHF₂, —CH₂CF₃, —CH₂CH₂—OH, —CH₂CH₂—N(CH₃)₂, —CH₂CH CH₃, —CH(CH₃)₂,

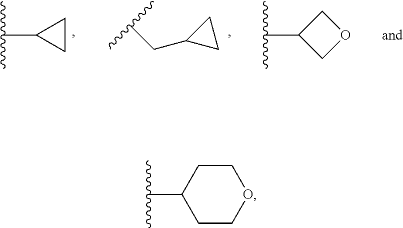

and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth, twentieth, twenty-first or twenty-second embodiment.

In a twenty-fourth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein each R⁴ is halo or OR^{a}; each R^{a} is independently selected from H and C₁-C₄alkyl; and n is 0, 1, 2 or 3, and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth, twentieth, twenty-first, twenty-second or twenty-third embodiment.

In a twenty-fifth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein n is 0, and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment.

In a twenty-sixth embodiment, the present disclosure provides a compound according to structural formula (A), (B), (C), (I), (II-1), (II-1a), (II-2), (II-3), (II-3a), (II-3b), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12) or (II-12a), or a pharmaceutically acceptable salt thereof, wherein n is 1 or 2, and each R⁴ is independently selected from F and OH, and wherein the remainder of the variables are as defined in the first, third, fourth, fifth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth or twenty-fifth embodiment.

In a twenty-seventh embodiment, the compound of formula (I) is represented by one of structural formula (II-3c) or (II-3b):

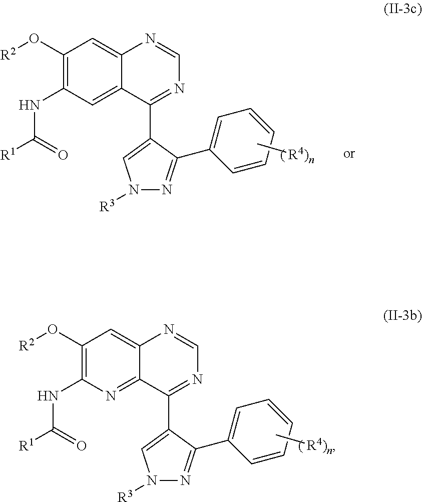

or a pharmaceutically acceptable salt thereof, wherein the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth or twenty-sixth embodiment.

In a twenty-eighth embodiment, the compound of formula (I) is represented by one of structural formula (II-7a) or (II-7b):

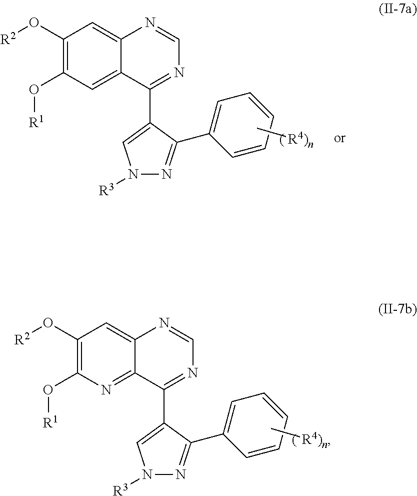

or a pharmaceutically acceptable salt thereof, wherein the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth or twenty-sixth embodiment.

In a twenty-ninth embodiment, the present disclosure provides a compound according to structural formula (II-3c), (II-3b), (II-7a) or (II-7b), or a pharmaceutically acceptable salt thereof, wherein R¹ is: C₁-C₄alkyl optionally substituted with 5 to 10 membered heteroaryl, wherein the 5 to 10 membered heteroaryl is optionally substituted with C₁-C₃alkyl, or C₃-C₈cycloalkyl, 4 to 12 membered heterocyclyl or 5 to 12 membered heteroaryl, wherein the cycloalkyl, heterocyclyl and heteroaryl represented by R¹ are each optionally substituted with 1 to 3 groups independently selected from R¹¹; each R¹¹ is independently selected from halo, NR^{a}R^{b}, C₃-C₆cycloalkyl, and C₁-C₄alkyl optionally substituted with 1 to 3 halo, and each R^{a} is independently selected from H and C₁-C₄alkyl; R² and R³ are each independently C₁-C₄alkyl; and R⁴ is halo, where n is 0, 1, or 2, and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth or twenty-sixth embodiment.

In a thirtieth embodiment, the present disclosure provides a compound according to structural formula (II-3c), (II-3b), (II-7a) or (II-7b), or a pharmaceutically acceptable salt thereof, wherein R¹ is: C₁-C₃alkyl optionally substituted with 5 membered heteroaryl wherein the 5 membered heteroaryl is optionally substituted with C₁-C₂alkyl, or C₃-C₈cycloalkyl, 5-7 membered heterocyclyl or 5-6 membered heteroaryl, wherein the cycloalkyl, heterocyclyl and heteroaryl represented by R¹ are each optionally substituted with 1 to 2 groups independently selected from R¹¹; each R¹¹ is independently selected from halo, N(CH₃)₂, C₃-C₈cycloalkyl, and C₁-C₂alkyl optionally substituted with 1 to 3 halo; R² and R³ are each independently C₁-C₃alkyl; R⁴ is halo; and n is 0, 1, or 2, and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth or twenth-ninth embodiment.

In a thirty-first embodiment, the present disclosure provides a compound according to structural formula (II-3c), (II-3b), (II-7a) or (II-7b), or a pharmaceutically acceptable salt thereof, wherein R¹ is —CH₂CH; substituted with oxadiazolyl optionally substituted with —CH₃; or R¹ is selected from azabicyclo[3.1.0]hexanyl, bicyclo[1.1.1]pentanyl, cyclopropyl, 3-oxabicyclo[3.1.0]hexanyl, piperidinyl, pyrazolyl and pyridinyl, each of which is optionally substituted with 1 to 2 groups independently selected from R¹¹, each R¹¹ is independently selected from F, —N(CH₃)₂, —CH₃, —CF₃,

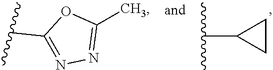

and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenth-ninth or thirtieth embodiment.

In a thirty-second embodiment, the present disclosure provides a compound according to structural formula (II-3c), (II-3b), (II-7a) or (II-7b), or a pharmaceutically acceptable salt thereof, wherein R¹ is selected from

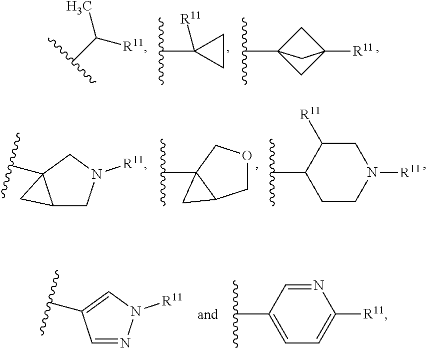

and each R¹¹ is independently selected from F, —N(CH₃)₂, —CH₃, —CF₃,

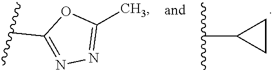

and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenth-ninth, thirtieth or thirty-first embodiment.

In a thirty-third embodiment, the present disclosure provides a compound according to structural formula (II-3c), (II-3b), (II-7a) or (II-7b), or a pharmaceutically acceptable salt thereof, wherein R² is C₁-C₄alkyl; R³ is C₁-C₄alkyl; each R⁴ is independently halo; and n is 0, 1 or 2, and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenth-ninth, thirtieth, thirty-first or thirty-second embodiment.

In a thirty-fourth embodiment, the present disclosure provides a compound according to structural formula (II-3c), (II-3b), (II-7a) or (II-7b), or a pharmaceutically acceptable salt thereof, wherein R² is —CH₃ or —CH₂CH₃; R³ is —CH₃; R⁴ is F; and n is 0 or 1, and wherein the remainder of the variables are as defined in the first, third, fourth, sixth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenth-ninth, thirtieth, thirty-first, thirty-second or thirty-third embodiment.

In a thirty-fifth embodiment, the compound of formula (I) is represented by the following structural formula (III):

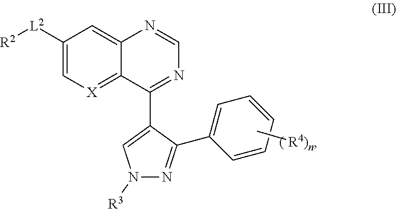

or a pharmaceutically acceptable salt thereof, and wherein the variables are as defined in the first, third, fourth, sixth, ninth, sixteenth, seventeenth, eighteenth, nineteenth, twentieth, twenty-first, twenty-second, twenty-third, twenty-fourth, twenty-fifth, twenty-sixth, thirty-third and thirty-fourth embodiment.

In a thirty-sixth embodiment, the compound of formula (I) is represented by one of the following structural formula (III-1) or (III-2):

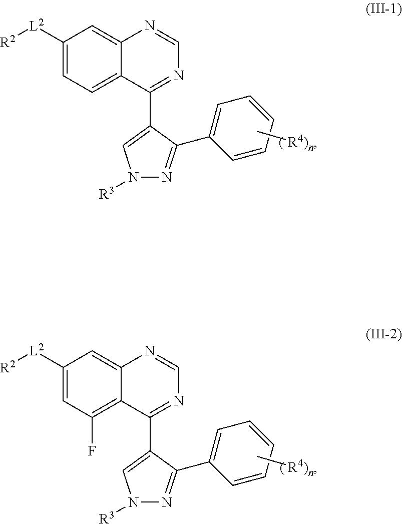

or a pharmaceutically acceptable salt thereof, and wherein the variables are as defined in the first, third, fourth, sixth, sixteenth, seventeenth, eighteenth, nineteenth, twentieth, twenty-first, twenty-second, twenty-third, twenty-fourth, twenty-fifth twenty-sixth, thirty-third and thirty-fourth embodiment.

In a thirty-seventh embodiment, the present disclosure provides a compound according to structural formula (III), (III-1) or (III-2), or a pharmaceutically acceptable salt thereof, wherein L² is a bond, and wherein the remainder of the variables are as defined in the first, third, fourth, sixth, ninth, sixteenth, seventeenth, eighteenth, nineteenth, twentieth, twenty-first, twenty-second, twenty-third, twenty-fourth, twenty-fifth, twenty-sixth, thirty-third and thirty-fourth embodiment.

In a thirty-eighth embodiment, a compound of the present disclosure is any one of the compounds disclosed in the examples (including neutral form, pharmaceutically acceptable salts, and intermediates) and Table 1, or a pharmaceutically acceptable salt thereof.

TABLE 1

| Example | |
|---|---|
| number | Structure |
| 1 | 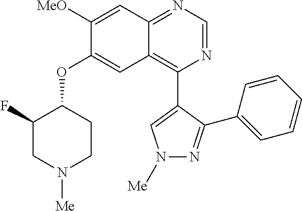 [SMILES: COc1cc2ncnc(-c3cn(C)nc3-c3ccccc3)c2cc1O[C@@H]1CCN(C)C[C@H]1F] |
| 2 | 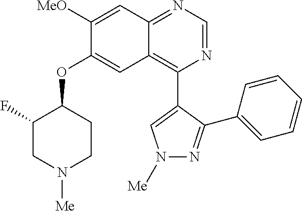 [SMILES: COc1cc2ncnc(-c3cn(C)nc3-c3ccccc3)c2cc1O[C@H]1CCN(C)C[C@@H]1F] |
| 3 | 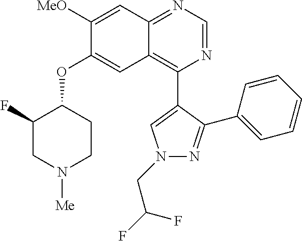 [SMILES: COc1cc2ncnc(-c3cn(CC(F)F)nc3-c3ccccc3)c2cc1O[C@@H]1CCN(C)C[C@H]1F] |
| 4 | 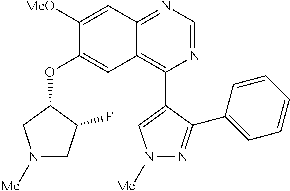 [SMILES: COc1cc2ncnc(-c3cn(C)nc3-c3ccccc3)c2cc1O[C@H]1CN(C)C[C@H]1F] |
| | or |
| | 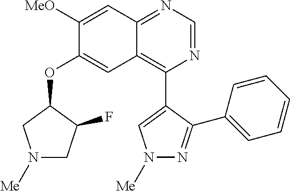 [SMILES: COc1cc2ncnc(-c3cn(C)nc3-c3ccccc3)c2cc1O[C@@H]1CN(C)C[C@@H]1F] |
| 5 | 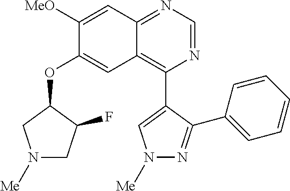 [SMILES: COc1cc2ncnc(-c3cn(C)nc3-c3ccccc3)c2cc1O[C@@H]1CN(C)C[C@@H]1F] |
| | or |
| | 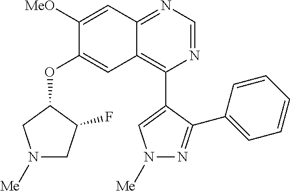 [SMILES: COc1cc2ncnc(-c3cn(C)nc3-c3ccccc3)c2cc1O[C@H]1CN(C)C[C@H]1F] |
| 6 | 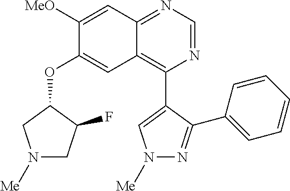 [SMILES: COc1cc2ncnc(-c3cn(C)nc3-c3ccccc3)c2cc1O[C@H]1CN(C)C[C@@H]1F] |
| | or |
| | 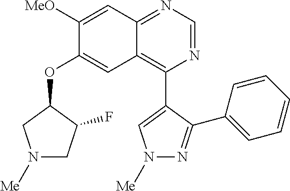 [SMILES: COc1cc2ncnc(-c3cn(C)nc3-c3ccccc3)c2cc1O[C@@H]1CN(C)C[C@H]1F] |
| 7 | 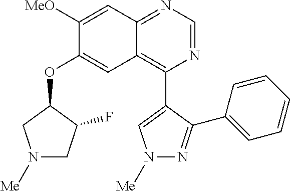 [SMILES: COc1cc2ncnc(-c3cn(C)nc3-c3ccccc3)c2cc1O[C@@H]1CN(C)C[C@H]1F] |
| | or |
| | 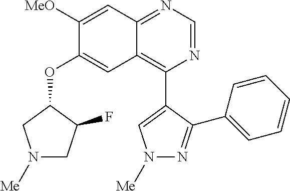 [SMILES: COc1cc2ncnc(-c3cn(C)nc3-c3ccccc3)c2cc1O[C@H]1CN(C)C[C@@H]1F] |
| 8 | 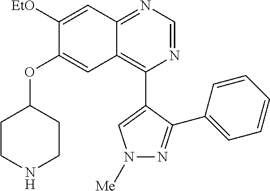 [SMILES: CCOc1cc2ncnc(-c3cn(C)nc3-c3ccccc3)c2cc1OC1CCNCC1] |
| 9 | 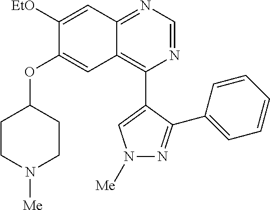 [SMILES: CCOc1cc2ncnc(-c3cn(C)nc3-c3ccccc3)c2cc1OC1CCN(C)CC1] |
| 10 | 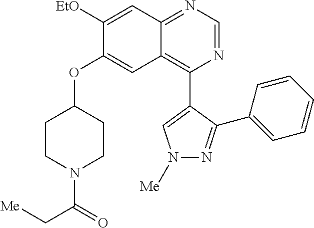 [SMILES: CCOc1cc2ncnc(-c3cn(C)nc3-c3ccccc3)c2cc1OC1CCN(C(=O)CC)CC1] |
| 11 | 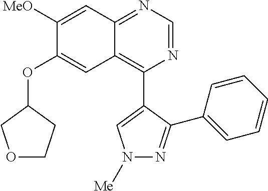 [SMILES: COc1cc2ncnc(-c3cn(C)nc3-c3ccccc3)c2cc1OC1CCOC1] |
| 12 | 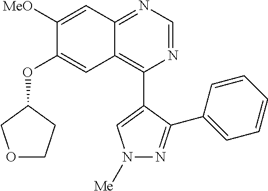 [SMILES: COc1cc2ncnc(-c3cn(C)nc3-c3ccccc3)c2cc1O[C@@H]1CCOC1] |
| | or |
| | 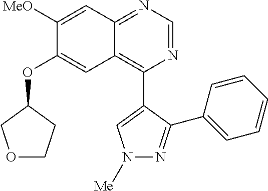 [SMILES: COc1cc2ncnc(-c3cn(C)nc3-c3ccccc3)c2cc1O[C@H]1CCOC1] |
| 13 | 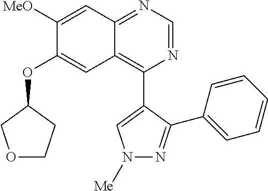 [SMILES: COc1cc2ncnc(-c3cn(C)nc3-c3ccccc3)c2cc1O[C@H]1CCOC1] |
| | or |
| | 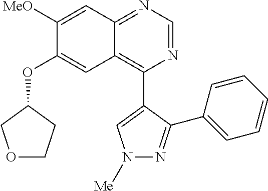 [SMILES: COc1cc2ncnc(-c3cn(C)nc3-c3ccccc3)c2cc1O[C@@H]1CCOC1] |
| 14 | 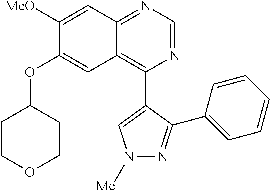 [SMILES: COc1cc2ncnc(-c3cn(C)nc3-c3ccccc3)c2cc1OC1CCOCC1] |
| 15 | 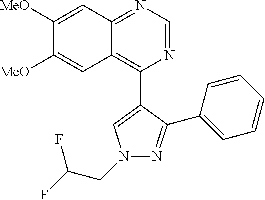 [SMILES: COc1cc2ncnc(-c3cn(CC(F)F)nc3-c3ccccc3)c2cc1OC] |
| 16 | 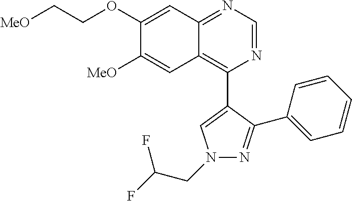 [SMILES: COCCOc1cc2ncnc(-c3cn(CC(F)F)nc3-c3ccccc3)c2cc1OC] |
| 17 | 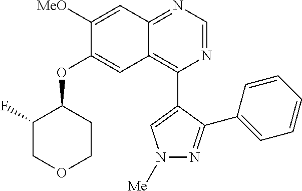 [SMILES: COc1cc2ncnc(-c3cn(C)nc3-c3ccccc3)c2cc1O[C@H]1CCOC[C@@H]1F] |
| | or |
| | 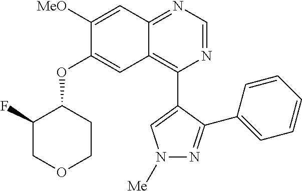 [SMILES: COc1cc2ncnc(-c3cn(C)nc3-c3ccccc3)c2cc1O[C@@H]1CCOC[C@H]1F] |
| 18 | 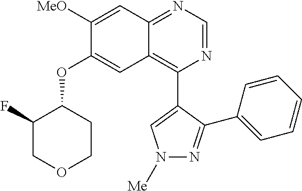 [SMILES: COc1cc2ncnc(-c3cn(C)nc3-c3ccccc3)c2cc1O[C@@H]1CCOC[C@H]1F] |
| | or |
| | 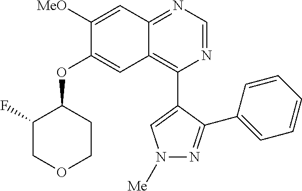 [SMILES: COc1cc2ncnc(-c3cn(C)nc3-c3ccccc3)c2cc1O[C@H]1CCOC[C@@H]1F] |
| 19 | 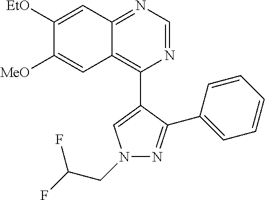 [SMILES: CCOc1cc2ncnc(-c3cn(CC(F)F)nc3-c3ccccc3)c2cc1OC] |
| 20 | 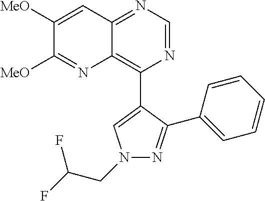 [SMILES: COc1cc2ncnc(-c3cn(CC(F)F)nc3-c3ccccc3)c2nc1OC] |
| 21 | 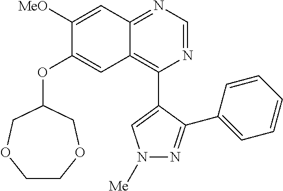 [SMILES: COc1cc2ncnc(-c3cn(C)nc3-c3ccccc3)c2cc1OC1COCCOC1] |
| 22 | 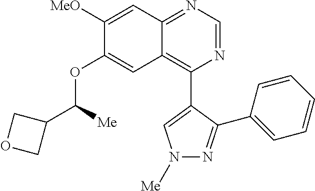 [SMILES: COc1cc2ncnc(-c3cn(C)nc3-c3ccccc3)c2cc1O[C@@H](C)C1COC1] |
| 23 | 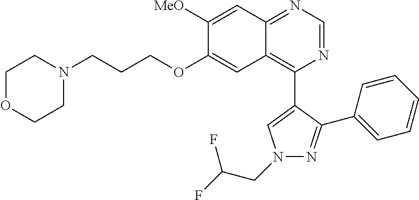 [SMILES: COc1cc2ncnc(-c3cn(CC(F)F)nc3-c3ccccc3)c2cc1OCCCN1CCOCC1] |
| 24 | 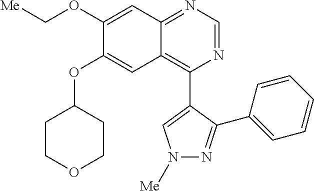 [SMILES: CCOc1cc2ncnc(-c3cn(C)nc3-c3ccccc3)c2cc1OC1CCOCC1] |
| 25 | 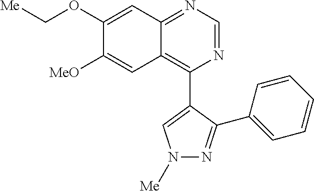 [SMILES: CCOc1cc2ncnc(-c3cn(C)nc3-c3ccccc3)c2cc1OC] |
| 26 | 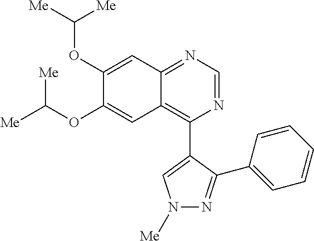 [SMILES: CC(C)Oc1cc2ncnc(-c3cn(C)nc3-c3ccccc3)c2cc1OC(C)C] |
| 27 | 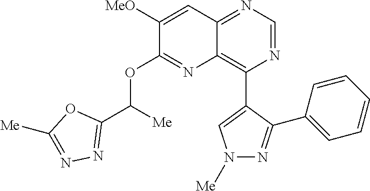 [SMILES: COc1cc2ncnc(-c3cn(C)nc3-c3ccccc3)c2nc1OC(C)c1nnc(C)o1] |
| 28 | 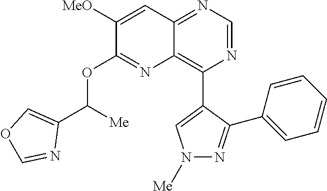 [SMILES: COc1cc2ncnc(-c3cn(C)nc3-c3ccccc3)c2nc1OC(C)c1cocn1] |
| 29 | 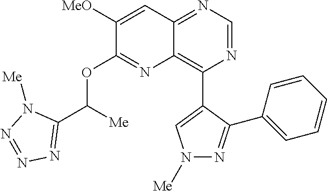 [SMILES: COc1cc2ncnc(-c3cn(C)nc3-c3ccccc3)c2nc1OC(C)c1nnnn1C] |
| 30 | 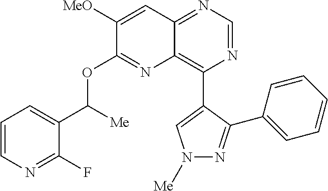 [SMILES: COc1cc2ncnc(-c3cn(C)nc3-c3ccccc3)c2nc1OC(C)c1cccnc1F] |
| 31 | 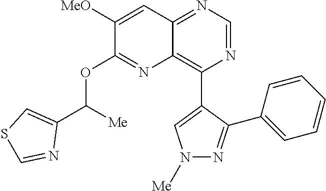 [SMILES: COc1cc2ncnc(-c3cn(C)nc3-c3ccccc3)c2nc1OC(C)c1cscn1] |
| 32 | 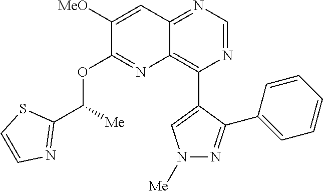 [SMILES: COc1cc2ncnc(-c3cn(C)nc3-c3ccccc3)c2nc1O[C@H](C)c1nccs1] |
| 33 | 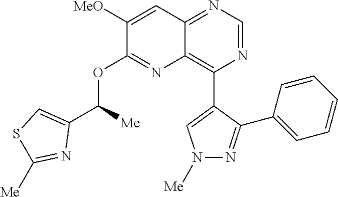 [SMILES: COc1cc2ncnc(-c3cn(C)nc3-c3ccccc3)c2nc1O[C@@H](C)c1csc(C)n1] |
| 34 | 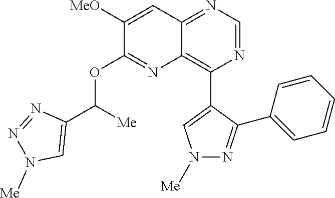 [SMILES: COc1cc2ncnc(-c3cn(C)nc3-c3ccccc3)c2nc1OC(C)c1cn(C)nn1] |
| 35 | 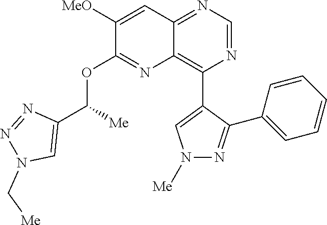 [SMILES: CCn1cc([C@@H](C)Oc2nc3c(-c4cn(C)nc4-c4ccccc4)ncnc3cc2OC)nn1] |
| | or |
| | 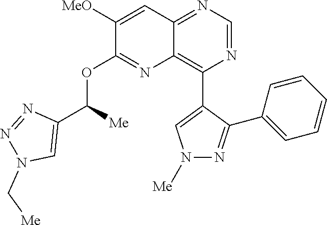 [SMILES: CCn1cc([C@H](C)Oc2nc3c(-c4cn(C)nc4-c4ccccc4)ncnc3cc2OC)nn1] |
| 36 | 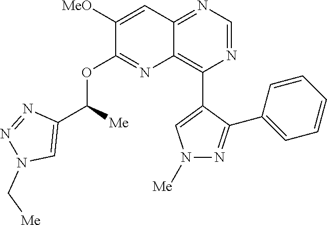 [SMILES: CCn1cc([C@H](C)Oc2nc3c(-c4cn(C)nc4-c4ccccc4)ncnc3cc2OC)nn1] |
| | or |
| | 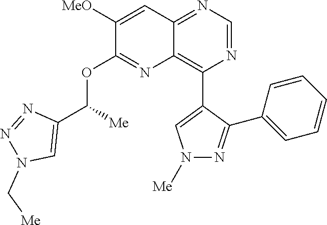 [SMILES: CCn1cc([C@@H](C)Oc2nc3c(-c4cn(C)nc4-c4ccccc4)ncnc3cc2OC)nn1] |
| 37 | 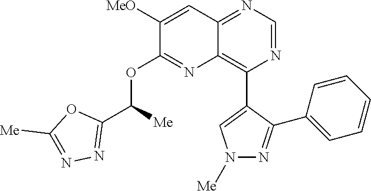 [SMILES: COc1cc2ncnc(-c3cn(C)nc3-c3ccccc3)c2nc1O[C@@H](C)c1nnc(C)o1] |
| | or |
| | 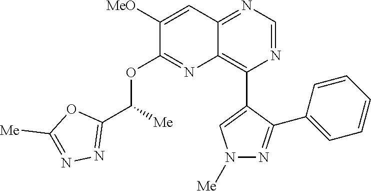 [SMILES: COc1cc2ncnc(-c3cn(C)nc3-c3ccccc3)c2nc1O[C@H](C)c1nnc(C)o1] |
| 38 | 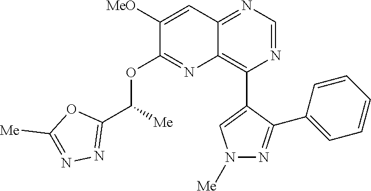 [SMILES: COc1cc2ncnc(-c3cn(C)nc3-c3ccccc3)c2nc1O[C@H](C)c1nnc(C)o1] |
| | or |
| | 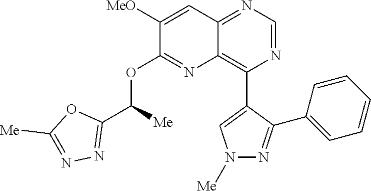 [SMILES: COc1cc2ncnc(-c3cn(C)nc3-c3ccccc3)c2nc1O[C@@H](C)c1nnc(C)o1] |
| 39 | 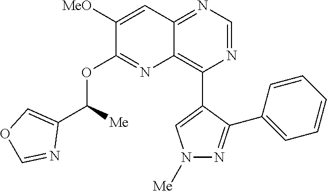 [SMILES: COc1cc2ncnc(-c3cn(C)nc3-c3ccccc3)c2nc1O[C@@H](C)c1cocn1] |
| | or |
| | 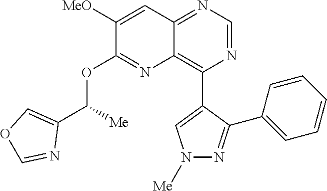 [SMILES: COc1cc2ncnc(-c3cn(C)nc3-c3ccccc3)c2nc1O[C@H](C)c1cocn1] |
| 40 | 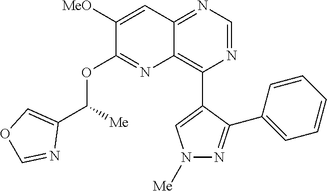 [SMILES: COc1cc2ncnc(-c3cn(C)nc3-c3ccccc3)c2nc1O[C@H](C)c1cocn1] |
| | or |
| | 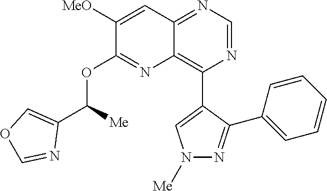 [SMILES: COc1cc2ncnc(-c3cn(C)nc3-c3ccccc3)c2nc1O[C@@H](C)c1cocn1] |
| 41 | 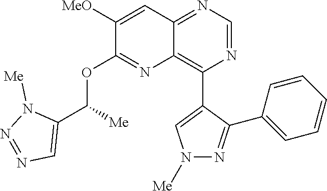 [SMILES: COc1cc2ncnc(-c3cn(C)nc3-c3ccccc3)c2nc1O[C@H](C)c1cnnn1C] |
| | Or |
| | 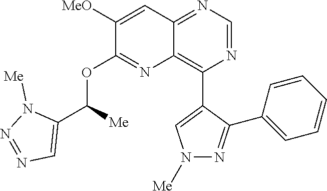 [SMILES: COc1cc2ncnc(-c3cn(C)nc3-c3ccccc3)c2nc1O[C@@H](C)c1cnnn1C] |
| 42 | 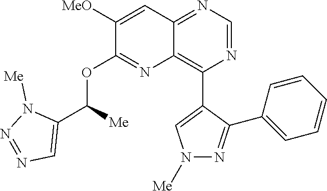 [SMILES: COc1cc2ncnc(-c3cn(C)nc3-c3ccccc3)c2nc1O[C@@H](C)c1cnnn1C] |
| | Or |
| | 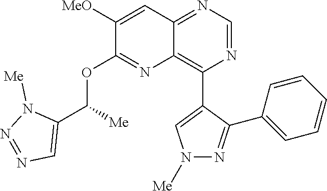 [SMILES: COc1cc2ncnc(-c3cn(C)nc3-c3ccccc3)c2nc1O[C@H](C)c1cnnn1C] |
| 43 | 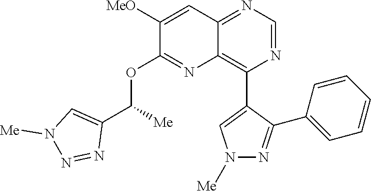 [SMILES: COc1cc2ncnc(-c3cn(C)nc3-c3ccccc3)c2nc1O[C@H](C)c1cn(C)nn1] |
| | Or |
| | 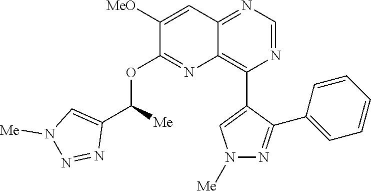 [SMILES: COc1cc2ncnc(-c3cn(C)nc3-c3ccccc3)c2nc1O[C@@H](C)c1cn(C)nn1] |
| 44 | 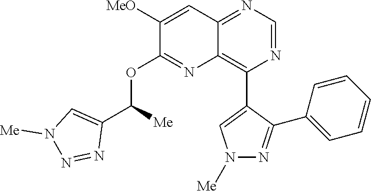 [SMILES: COc1cc2ncnc(-c3cn(C)nc3-c3ccccc3)c2nc1O[C@@H](C)c1cn(C)nn1] |
| | Or |
| | 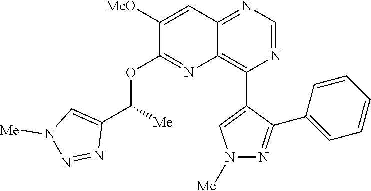 [SMILES: COc1cc2ncnc(-c3cn(C)nc3-c3ccccc3)c2nc1O[C@H](C)c1cn(C)nn1] |
| 45 | 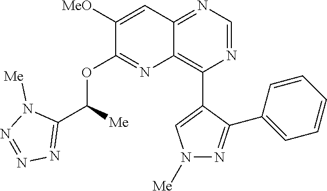 [SMILES: COc1cc2ncnc(-c3cn(C)nc3-c3ccccc3)c2nc1O[C@@H](C)c1nnnn1C] |
| | Or |
| | 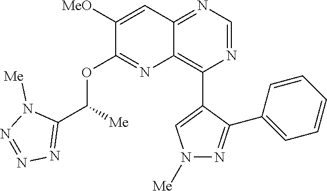 [SMILES: COc1cc2ncnc(-c3cn(C)nc3-c3ccccc3)c2nc1O[C@H](C)c1nnnn1C] |
| 46 | 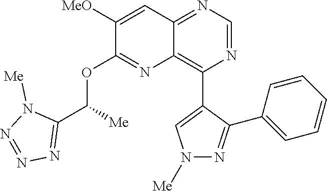 [SMILES: COc1cc2ncnc(-c3cn(C)nc3-c3ccccc3)c2nc1O[C@H](C)c1nnnn1C] |
| | Or |
| | 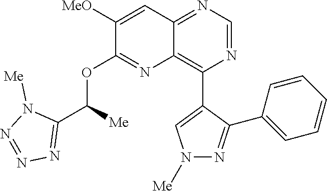 [SMILES: COc1cc2ncnc(-c3cn(C)nc3-c3ccccc3)c2nc1O[C@@H](C)c1nnnn1C] |
| 47 | 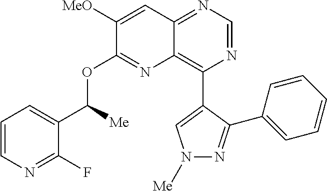 [SMILES: COc1cc2ncnc(-c3cn(C)nc3-c3ccccc3)c2nc1O[C@@H](C)c1cccnc1F] |
| | or |
| | 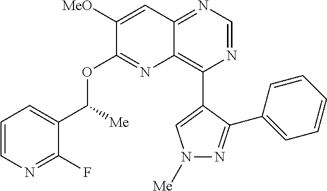 [SMILES: COc1cc2ncnc(-c3cn(C)nc3-c3ccccc3)c2nc1O[C@H](C)c1cccnc1F] |
| 48 | 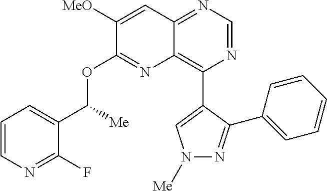 [SMILES: COc1cc2ncnc(-c3cn(C)nc3-c3ccccc3)c2nc1O[C@H](C)c1cccnc1F] |
| | or |
| | 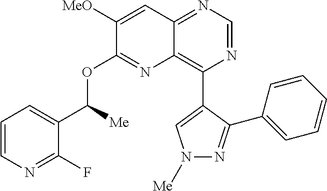 [SMILES: COc1cc2ncnc(-c3cn(C)nc3-c3ccccc3)c2nc1O[C@@H](C)c1cccnc1F] |
| 49 | 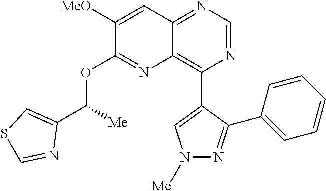 [SMILES: COc1cc2ncnc(-c3cn(C)nc3-c3ccccc3)c2nc1O[C@H](C)c1cscn1] |
| | Or |
| | 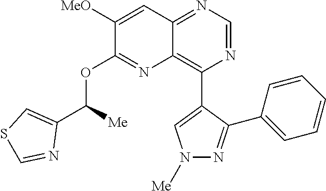 [SMILES: COc1cc2ncnc(-c3cn(C)nc3-c3ccccc3)c2nc1O[C@@H](C)c1cscn1] |
| 50 | 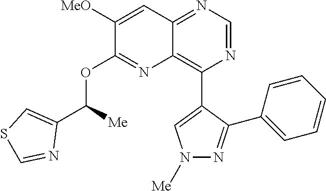 [SMILES: COc1cc2ncnc(-c3cn(C)nc3-c3ccccc3)c2nc1O[C@@H](C)c1cscn1] |
| | Or |
| | 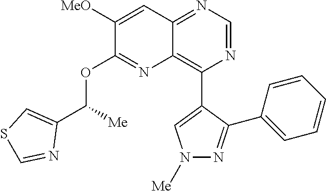 [SMILES: COc1cc2ncnc(-c3cn(C)nc3-c3ccccc3)c2nc1O[C@H](C)c1cscn1] |
| 51 | 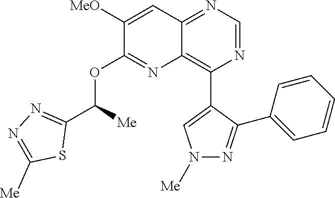 [SMILES: COc1cc2ncnc(-c3cn(C)nc3-c3ccccc3)c2nc1O[C@@H](C)c1nnc(C)s1] |
| | Or |
| | 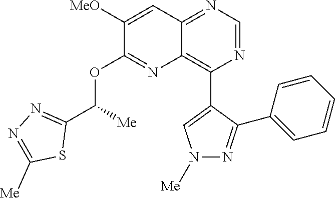 [SMILES: COc1cc2ncnc(-c3cn(C)nc3-c3ccccc3)c2nc1O[C@H](C)c1nnc(C)s1] |
| 52 | 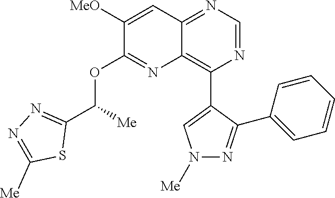 [SMILES: COc1cc2ncnc(-c3cn(C)nc3-c3ccccc3)c2nc1O[C@H](C)c1nnc(C)s1] |
| | Or |
| | 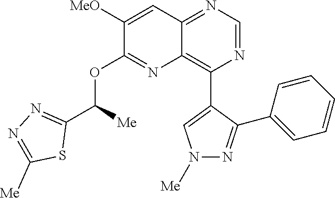 [SMILES: COc1cc2ncnc(-c3cn(C)nc3-c3ccccc3)c2nc1O[C@@H](C)c1nnc(C)s1] |
| 53 | 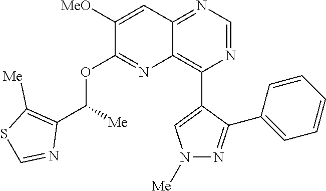 [SMILES: COc1cc2ncnc(-c3cn(C)nc3-c3ccccc3)c2nc1O[C@H](C)c1ncsc1C] |
| | Or |
| | 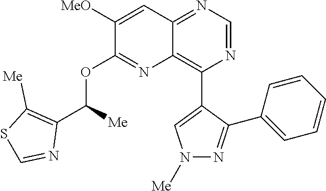 [SMILES: COc1cc2ncnc(-c3cn(C)nc3-c3ccccc3)c2nc1O[C@@H](C)c1ncsc1C] |
| 54 | 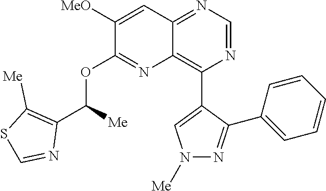 [SMILES: COc1cc2ncnc(-c3cn(C)nc3-c3ccccc3)c2nc1O[C@@H](C)c1ncsc1C] |
| | Or |
| | 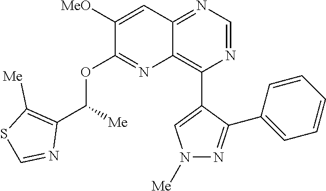 [SMILES: COc1cc2ncnc(-c3cn(C)nc3-c3ccccc3)c2nc1O[C@H](C)c1ncsc1C] |
| 55 | 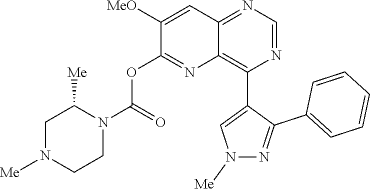 [SMILES: COc1cc2ncnc(-c3cn(C)nc3-c3ccccc3)c2nc1OC(=O)N1CCN(C)C[C@@H]1C] |
| 56 | 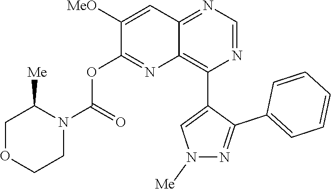 [SMILES: COc1cc2ncnc(-c3cn(C)nc3-c3ccccc3)c2nc1OC(=O)N1CCOC[C@H]1C] |
| 57 | 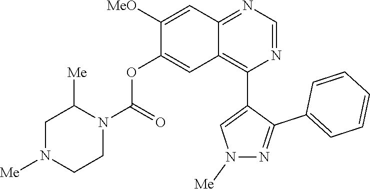 [SMILES: COc1cc2ncnc(-c3cn(C)nc3-c3ccccc3)c2cc1OC(=O)N1CCN(C)CC1C] |
| 58 | 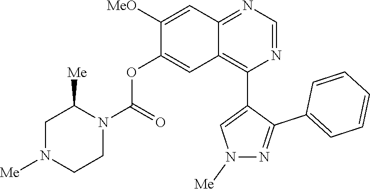 [SMILES: COc1cc2ncnc(-c3cn(C)nc3-c3ccccc3)c2cc1OC(=O)N1CCN(C)C[C@H]1C] |
| | Or |
| | 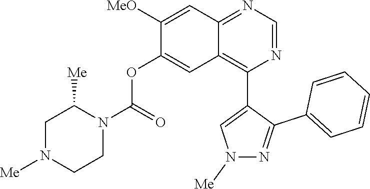 [SMILES: COc1cc2ncnc(-c3cn(C)nc3-c3ccccc3)c2cc1OC(=O)N1CCN(C)C[C@@H]1C] |
| 59 | 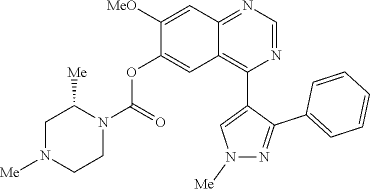 [SMILES: COc1cc2ncnc(-c3cn(C)nc3-c3ccccc3)c2cc1OC(=O)N1CCN(C)C[C@@H]1C] |
| | Or |
| | 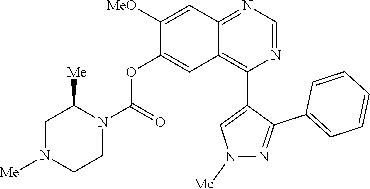 [SMILES: COc1cc2ncnc(-c3cn(C)nc3-c3ccccc3)c2cc1OC(=O)N1CCN(C)C[C@H]1C] |
| 60 | 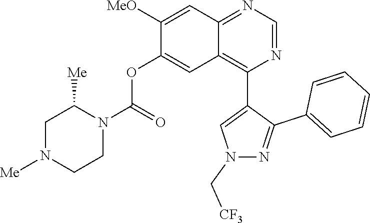 [SMILES: COc1cc2ncnc(-c3cn(CC(F)(F)F)nc3-c3ccccc3)c2cc1OC(=O)N1CCN(C)C[C@@H]1C] |
| 61 | 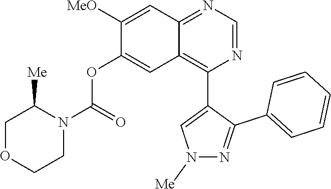 [SMILES: COc1cc2ncnc(-c3cn(C)nc3-c3ccccc3)c2cc1OC(=O)N1CCOC[C@H]1C] |
| 62 | 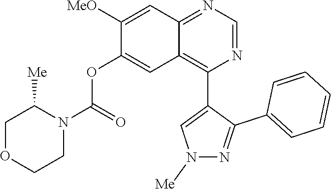 [SMILES: COc1cc2ncnc(-c3cn(C)nc3-c3ccccc3)c2cc1OC(=O)N1CCOC[C@@H]1C] |
| 63 | 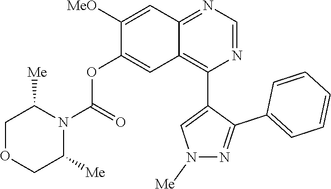 [SMILES: COc1cc2ncnc(-c3cn(C)nc3-c3ccccc3)c2cc1OC(=O)N1[C@H](C)COC[C@@H]1C] |
| 64 | 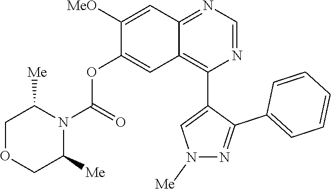 [SMILES: COc1cc2ncnc(-c3cn(C)nc3-c3ccccc3)c2cc1OC(=O)N1[C@@H](C)COC[C@@H]1C] |
| 65 | 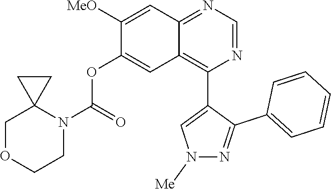 [SMILES: COc1cc2ncnc(-c3cn(C)nc3-c3ccccc3)c2cc1OC(=O)N1CCOCC12CC2] |
| 66 | 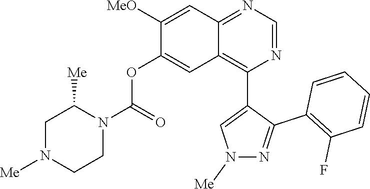 [SMILES: COc1cc2ncnc(-c3cn(C)nc3-c3ccccc3F)c2cc1OC(=O)N1CCN(C)C[C@@H]1C] |
| 67 | 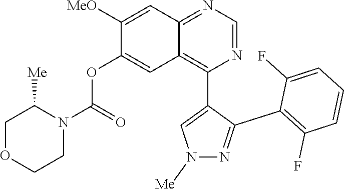 [SMILES: COc1cc2ncnc(-c3cn(C)nc3-c3c(F)cccc3F)c2cc1OC(=O)N1CCOC[C@@H]1C] |
| 68 | 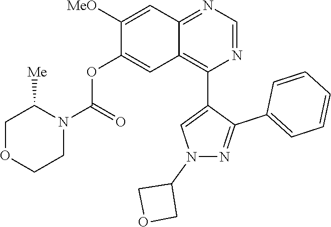 [SMILES: COc1cc2ncnc(-c3cn(C4COC4)nc3-c3ccccc3)c2cc1OC(=O)N1CCOC[C@@H]1C] |
| 69 | 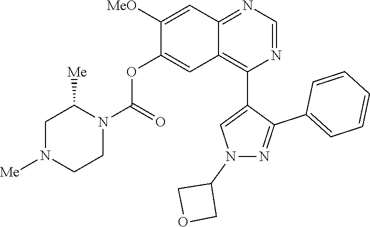 [SMILES: COc1cc2ncnc(-c3cn(C4COC4)nc3-c3ccccc3)c2cc1OC(=O)N1CCN(C)C[C@@H]1C] |
| 70 | 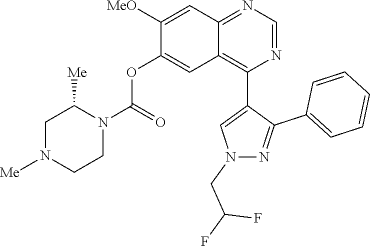 [SMILES: COc1cc2ncnc(-c3cn(CC(F)F)nc3-c3ccccc3)c2cc1OC(=O)N1CCN(C)C[C@@H]1C] |
| 71 | 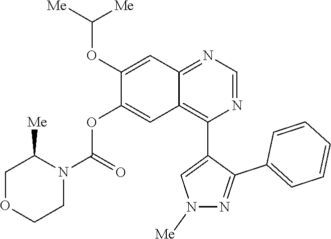 [SMILES: CC(C)Oc1cc2ncnc(-c3cn(C)nc3-c3ccccc3)c2cc1OC(=O)N1CCOC[C@H]1C] |
| 72 | 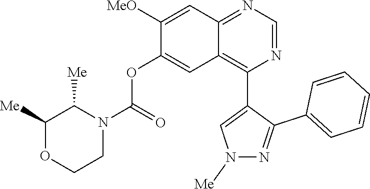 [SMILES: COc1cc2ncnc(-c3cn(C)nc3-c3ccccc3)c2cc1OC(=O)N1CCO[C@@H](C)[C@@H]1C] |
| 73 | 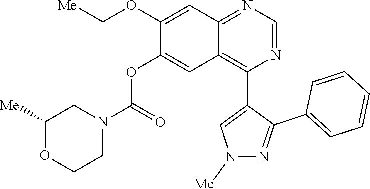 [SMILES: CCOc1cc2ncnc(-c3cn(C)nc3-c3ccccc3)c2cc1OC(=O)N1CCO[C@H](C)C1] |
| 74 | 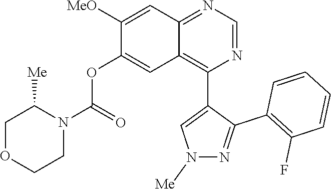 [SMILES: COc1cc2ncnc(-c3cn(C)nc3-c3ccccc3F)c2cc1OC(=O)N1CCOC[C@@H]1C] |
| 75 | 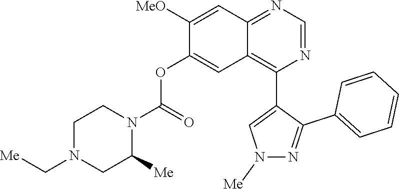 [SMILES: CCN1CCN(C(=O)Oc2cc3c(-c4cn(C)nc4-c4ccccc4)ncnc3cc2OC)[C@@H](C)C1] |
| 76 | 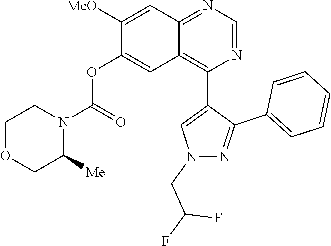 [SMILES: COc1cc2ncnc(-c3cn(CC(F)F)nc3-c3ccccc3)c2cc1OC(=O)N1CCOC[C@@H]1C] |
| 77 | 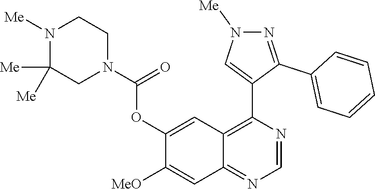 [SMILES: COc1cc2ncnc(-c3cn(C)nc3-c3ccccc3)c2cc1OC(=O)N1CCN(C)C(C)(C)C1] |
| 78 | 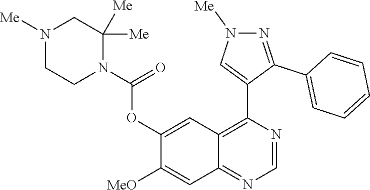 [SMILES: COc1cc2ncnc(-c3cn(C)nc3-c3ccccc3)c2cc1OC(=O)N1CCN(C)CC1(C)C] |
| 79 | 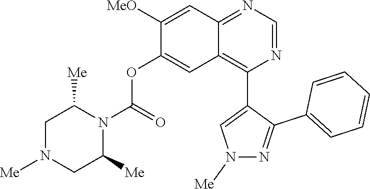 [SMILES: COc1cc2ncnc(-c3cn(C)nc3-c3ccccc3)c2cc1OC(=O)N1[C@@H](C)CN(C)C[C@@H]1C] |
| 80 | 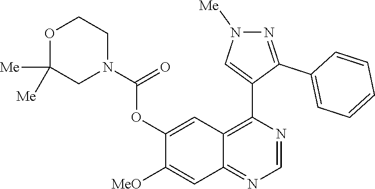 [SMILES: COc1cc2ncnc(-c3cn(C)nc3-c3ccccc3)c2cc1OC(=O)N1CCOC(C)(C)C1] |
| 81 | 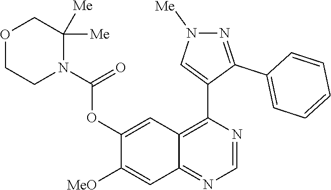 [SMILES: COc1cc2ncnc(-c3cn(C)nc3-c3ccccc3)c2cc1OC(=O)N1CCOCC1(C)C] |
| 82 | 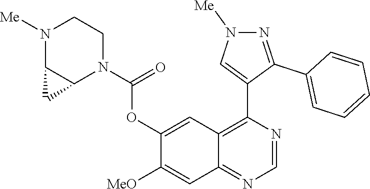 [SMILES: COc1cc2ncnc(-c3cn(C)nc3-c3ccccc3)c2cc1OC(=O)N1CCN(C)[C@H]2C[C@H]21] |
| 83 | 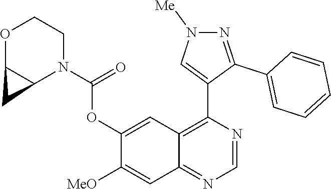 [SMILES: COc1cc2ncnc(-c3cn(C)nc3-c3ccccc3)c2cc1OC(=O)N1CCO[C@@H]2C[C@@H]21] |
| 84 | 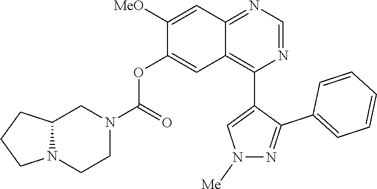 [SMILES: COc1cc2ncnc(-c3cn(C)nc3-c3ccccc3)c2cc1OC(=O)N1CCN2CCC[C@@H]2C1] |
| 85 | 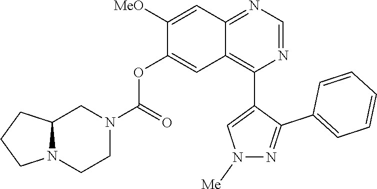 [SMILES: COc1cc2ncnc(-c3cn(C)nc3-c3ccccc3)c2cc1OC(=O)N1CCN2CCC[C@H]2C1] |
| 86 | 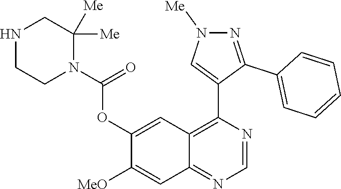 [SMILES: COc1cc2ncnc(-c3cn(C)nc3-c3ccccc3)c2cc1OC(=O)N1CCNCC1(C)C] |
| 87 | 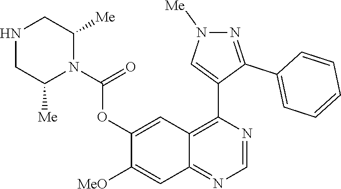 [SMILES: COc1cc2ncnc(-c3cn(C)nc3-c3ccccc3)c2cc1OC(=O)N1[C@H](C)CNC[C@@H]1C] |
| 88 | 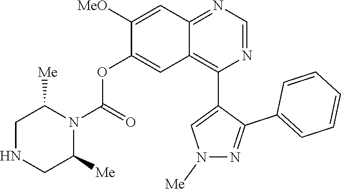 [SMILES: COc1cc2ncnc(-c3cn(C)nc3-c3ccccc3)c2cc1OC(=O)N1[C@@H](C)CNC[C@@H]1C] |
| 89 | 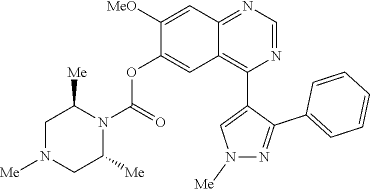 [SMILES: COc1cc2ncnc(-c3cn(C)nc3-c3ccccc3)c2cc1OC(=O)N1[C@H](C)CN(C)C[C@H]1C] |
| 90 | 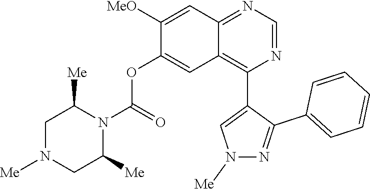 [SMILES: COc1cc2ncnc(-c3cn(C)nc3-c3ccccc3)c2cc1OC(=O)N1[C@H](C)CN(C)C[C@@H]1C] |
| 91 | 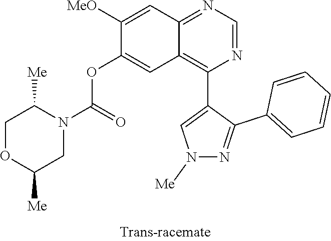 [SMILES: COc1cc2ncnc(-c3cn(C)nc3-c3ccccc3)c2cc1OC(=O)N1C[C@@H](C)OC[C@@H]1C] |
| | or |
| | 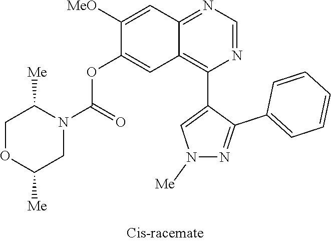 [SMILES: COc1cc2ncnc(-c3cn(C)nc3-c3ccccc3)c2cc1OC(=O)N1C[C@H](C)OC[C@@H]1C] |
| 92 | 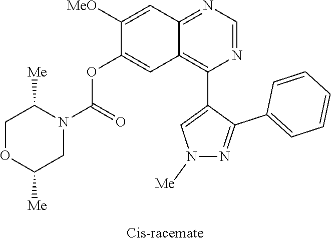 [SMILES: COc1cc2ncnc(-c3cn(C)nc3-c3ccccc3)c2cc1OC(=O)N1C[C@H](C)OC[C@@H]1C] |
| | or |
| | 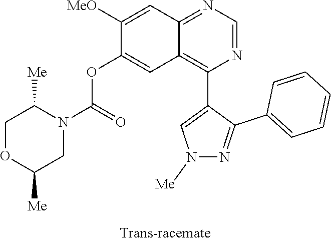 [SMILES: COc1cc2ncnc(-c3cn(C)nc3-c3ccccc3)c2cc1OC(=O)N1C[C@@H](C)OC[C@@H]1C] |
| 93 | 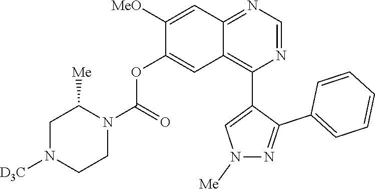 [SMILES: [2H]C([2H])([2H])N1CCN(C(=O)Oc2cc3c(-c4cn(C)nc4-c4ccccc4)ncnc3cc2OC)[C@@H](C)C1] |
| 94 | 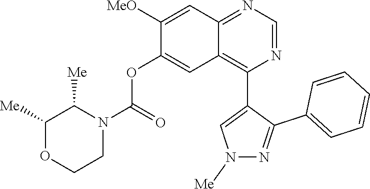 [SMILES: COc1cc2ncnc(-c3cn(C)nc3-c3ccccc3)c2cc1OC(=O)N1CCO[C@H](C)[C@@H]1C] |
| 95 | 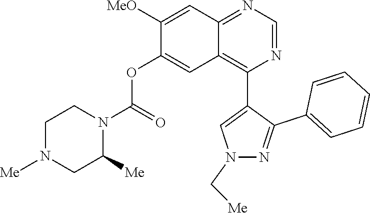 [SMILES: CCn1cc(-c2ncnc3cc(OC)c(OC(=O)N4CCN(C)C[C@@H]4C)cc23)c(-c2ccccc2)n1] |
| 96 | 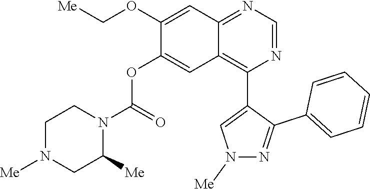 [SMILES: CCOc1cc2ncnc(-c3cn(C)nc3-c3ccccc3)c2cc1OC(=O)N1CCN(C)C[C@@H]1C] |
| 97 | 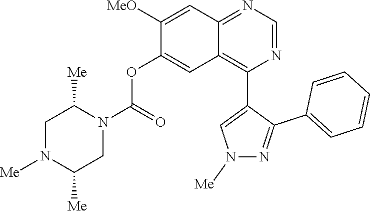 [SMILES: COc1cc2ncnc(-c3cn(C)nc3-c3ccccc3)c2cc1OC(=O)N1C[C@H](C)N(C)C[C@@H]1C] |
| 98 | 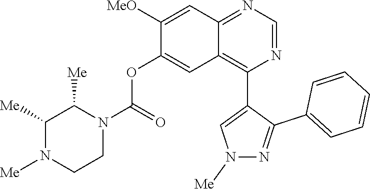 [SMILES: COc1cc2ncnc(-c3cn(C)nc3-c3ccccc3)c2cc1OC(=O)N1CCN(C)[C@H](C)[C@@H]1C] |
| 99 | 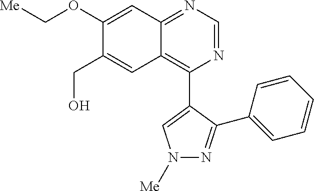 [SMILES: CCOc1cc2ncnc(-c3cn(C)nc3-c3ccccc3)c2cc1CO] |
| 100 | 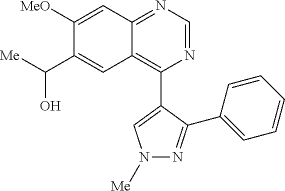 [SMILES: COc1cc2ncnc(-c3cn(C)nc3-c3ccccc3)c2cc1C(C)O] |
| 101 | 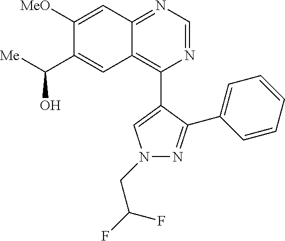 [SMILES: COc1cc2ncnc(-c3cn(CC(F)F)nc3-c3ccccc3)c2cc1[C@H](C)O] |
| | or |
| | 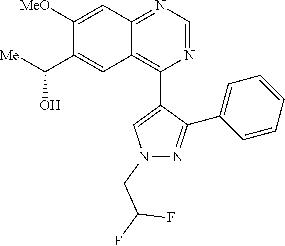 [SMILES: COc1cc2ncnc(-c3cn(CC(F)F)nc3-c3ccccc3)c2cc1[C@@H](C)O] |
| 102 | 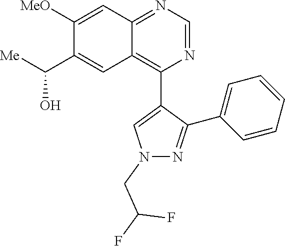 [SMILES: COc1cc2ncnc(-c3cn(CC(F)F)nc3-c3ccccc3)c2cc1[C@@H](C)O] |
| | or |
| | 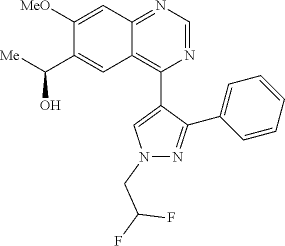 [SMILES: COc1cc2ncnc(-c3cn(CC(F)F)nc3-c3ccccc3)c2cc1[C@H](C)O] |
| 103 | 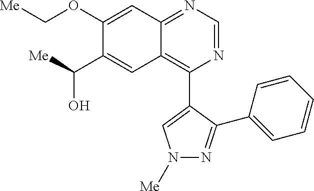 [SMILES: CCOc1cc2ncnc(-c3cn(C)nc3-c3ccccc3)c2cc1[C@H](C)O] |
| | or |
| | 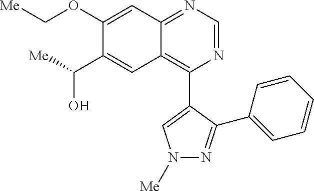 [SMILES: CCOc1cc2ncnc(-c3cn(C)nc3-c3ccccc3)c2cc1[C@@H](C)O] |
| 104 | 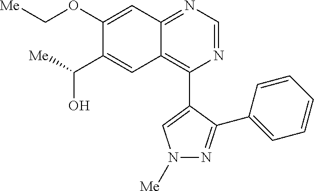 [SMILES: CCOc1cc2ncnc(-c3cn(C)nc3-c3ccccc3)c2cc1[C@@H](C)O] |
| | or |
| | 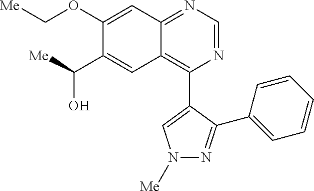 [SMILES: CCOc1cc2ncnc(-c3cn(C)nc3-c3ccccc3)c2cc1[C@H](C)O] |
| 105 | 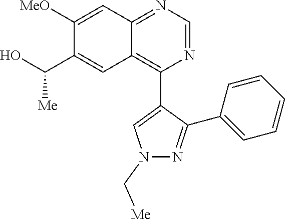 [SMILES: CCn1cc(-c2ncnc3cc(OC)c([C@H](C)O)cc23)c(-c2ccccc2)n1] |
| | or |
| | 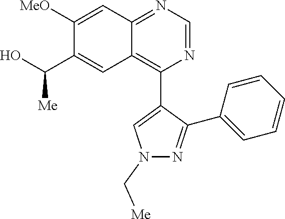 [SMILES: CCn1cc(-c2ncnc3cc(OC)c([C@@H](C)O)cc23)c(-c2ccccc2)n1] |
| 106 | 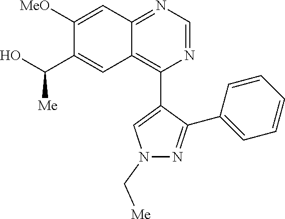 [SMILES: CCn1cc(-c2ncnc3cc(OC)c([C@@H](C)O)cc23)c(-c2ccccc2)n1] |
| | or |
| | 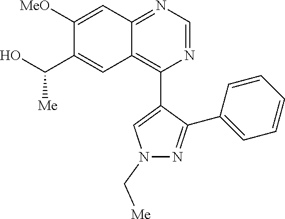 [SMILES: CCn1cc(-c2ncnc3cc(OC)c([C@H](C)O)cc23)c(-c2ccccc2)n1] |
| 107 | 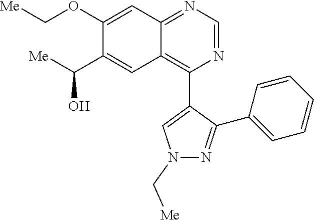 [SMILES: CCOc1cc2ncnc(-c3cn(CC)nc3-c3ccccc3)c2cc1[C@H](C)O] |
| | or |
| | 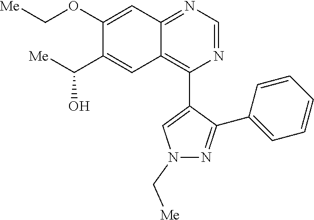 [SMILES: CCOc1cc2ncnc(-c3cn(CC)nc3-c3ccccc3)c2cc1[C@@H](C)O] |
| 108 | 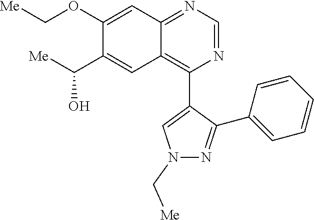 [SMILES: CCOc1cc2ncnc(-c3cn(CC)nc3-c3ccccc3)c2cc1[C@@H](C)O] |
| | or |
| | 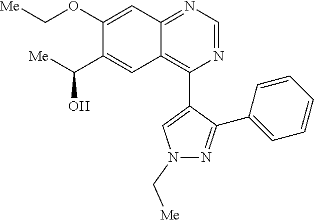 [SMILES: CCOc1cc2ncnc(-c3cn(CC)nc3-c3ccccc3)c2cc1[C@H](C)O] |
| 109 | 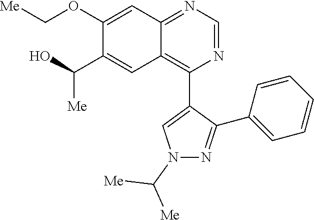 [SMILES: CCOc1cc2ncnc(-c3cn(C(C)C)nc3-c3ccccc3)c2cc1[C@@H](C)O] |
| | or |
| | 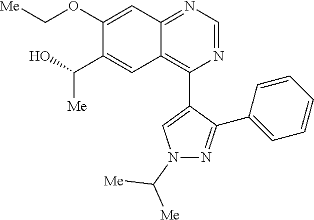 [SMILES: CCOc1cc2ncnc(-c3cn(C(C)C)nc3-c3ccccc3)c2cc1[C@H](C)O] |
| 110 | 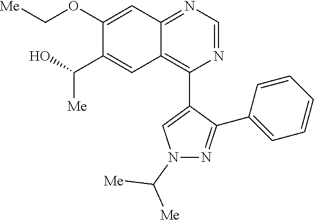 [SMILES: CCOc1cc2ncnc(-c3cn(C(C)C)nc3-c3ccccc3)c2cc1[C@H](C)O] |
| | or |
| | 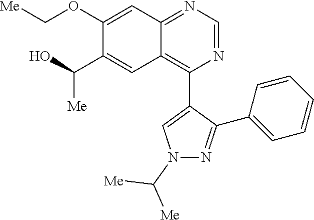 [SMILES: CCOc1cc2ncnc(-c3cn(C(C)C)nc3-c3ccccc3)c2cc1[C@@H](C)O] |
| 111 | 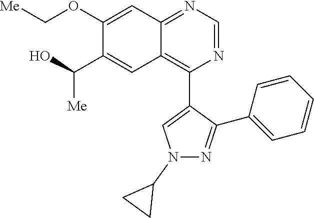 [SMILES: CCOc1cc2ncnc(-c3cn(C4CC4)nc3-c3ccccc3)c2cc1[C@@H](C)O] |
| | or |
| | 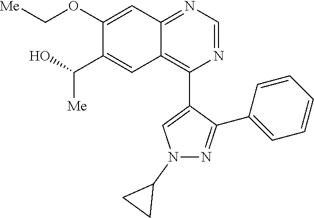 [SMILES: CCOc1cc2ncnc(-c3cn(C4CC4)nc3-c3ccccc3)c2cc1[C@H](C)O] |
| 112 | 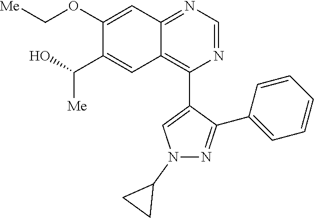 [SMILES: CCOc1cc2ncnc(-c3cn(C4CC4)nc3-c3ccccc3)c2cc1[C@H](C)O] |
| | or |
| | 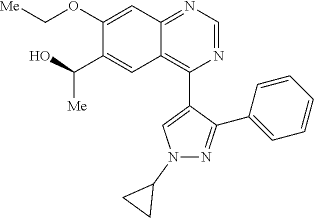 [SMILES: CCOc1cc2ncnc(-c3cn(C4CC4)nc3-c3ccccc3)c2cc1[C@@H](C)O] |
| 113 | 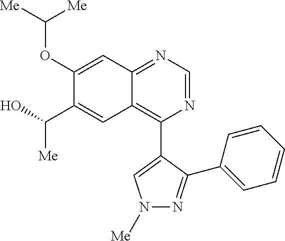 [SMILES: CC(C)Oc1cc2ncnc(-c3cn(C)nc3-c3ccccc3)c2cc1[C@H](C)O] |
| | or |
| | 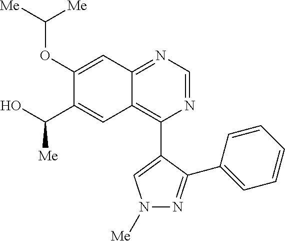 [SMILES: CC(C)Oc1cc2ncnc(-c3cn(C)nc3-c3ccccc3)c2cc1[C@@H](C)O] |
| 114 | 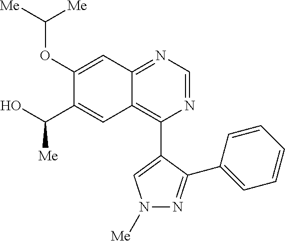 [SMILES: CC(C)Oc1cc2ncnc(-c3cn(C)nc3-c3ccccc3)c2cc1[C@@H](C)O] |
| | or |
| | 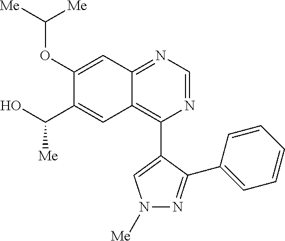 [SMILES: CC(C)Oc1cc2ncnc(-c3cn(C)nc3-c3ccccc3)c2cc1[C@H](C)O] |
| 115 | 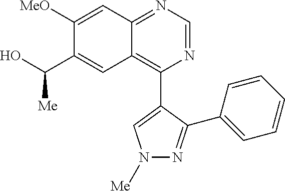 [SMILES: COc1cc2ncnc(-c3cn(C)nc3-c3ccccc3)c2cc1[C@@H](C)O] |
| | or |
| | 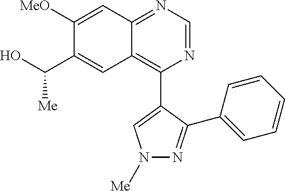 [SMILES: COc1cc2ncnc(-c3cn(C)nc3-c3ccccc3)c2cc1[C@H](C)O] |
| 116 | 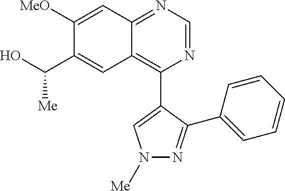 [SMILES: COc1cc2ncnc(-c3cn(C)nc3-c3ccccc3)c2cc1[C@H](C)O] |
| | or |
| | 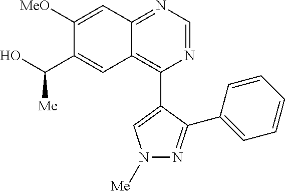 [SMILES: COc1cc2ncnc(-c3cn(C)nc3-c3ccccc3)c2cc1[C@@H](C)O] |
| 117 | 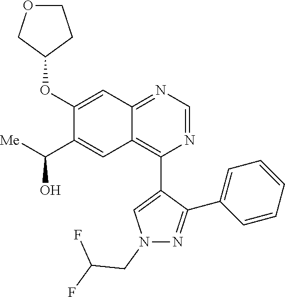 [SMILES: C[C@H](O)c1cc2c(-c3cn(CC(F)F)nc3-c3ccccc3)ncnc2cc1O[C@H]1CCOC1] |
| | or |
| | 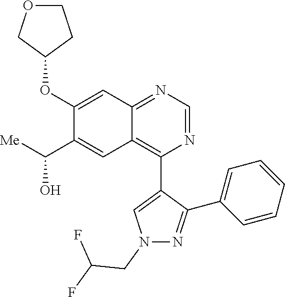 [SMILES: C[C@@H](O)c1cc2c(-c3cn(CC(F)F)nc3-c3ccccc3)ncnc2cc1O[C@H]1CCOC1] |
| 118 | 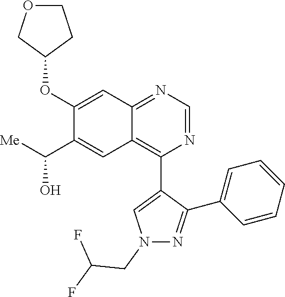 [SMILES: C[C@@H](O)c1cc2c(-c3cn(CC(F)F)nc3-c3ccccc3)ncnc2cc1O[C@H]1CCOC1] |
| | or |
| | 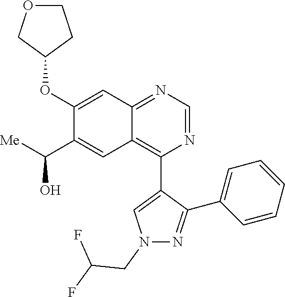 [SMILES: C[C@H](O)c1cc2c(-c3cn(CC(F)F)nc3-c3ccccc3)ncnc2cc1O[C@H]1CCOC1] |
| 119 | 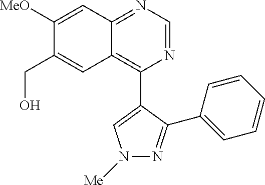 [SMILES: COc1cc2ncnc(-c3cn(C)nc3-c3ccccc3)c2cc1CO] |
| 120 | 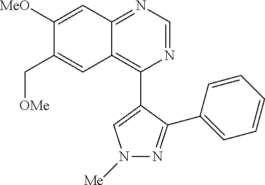 [SMILES: COCc1cc2c(-c3cn(C)nc3-c3ccccc3)ncnc2cc1OC] |
| 121 | 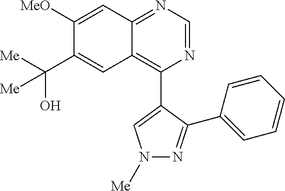 [SMILES: COc1cc2ncnc(-c3cn(C)nc3-c3ccccc3)c2cc1C(C)(C)O] |
| 122 | 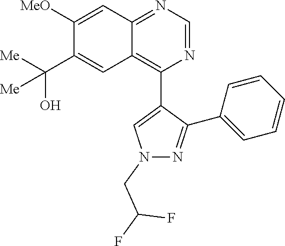 [SMILES: COc1cc2ncnc(-c3cn(CC(F)F)nc3-c3ccccc3)c2cc1C(C)(C)O] |
| 123 | 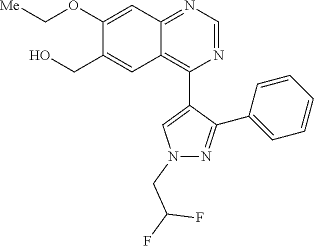 [SMILES: CCOc1cc2ncnc(-c3cn(CC(F)F)nc3-c3ccccc3)c2cc1CO] |
| 124 | 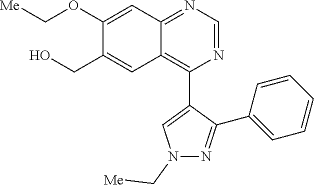 [SMILES: CCOc1cc2ncnc(-c3cn(CC)nc3-c3ccccc3)c2cc1CO] |
| 125 | 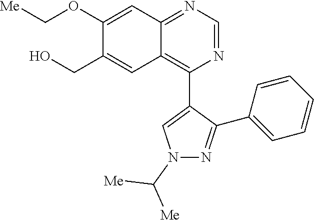 [SMILES: CCOc1cc2ncnc(-c3cn(C(C)C)nc3-c3ccccc3)c2cc1CO] |
| 126 | 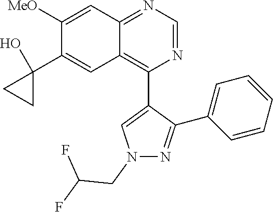 [SMILES: COc1cc2ncnc(-c3cn(CC(F)F)nc3-c3ccccc3)c2cc1C1(O)CC1] |
| 127 | 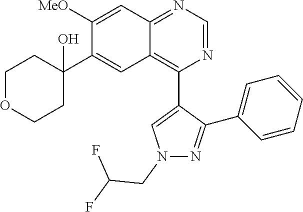 [SMILES: COc1cc2ncnc(-c3cn(CC(F)F)nc3-c3ccccc3)c2cc1C1(O)CCOCC1] |
| 128 | 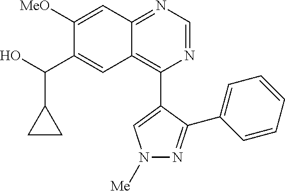 [SMILES: COc1cc2ncnc(-c3cn(C)nc3-c3ccccc3)c2cc1C(O)C1CC1] |
| 129 | 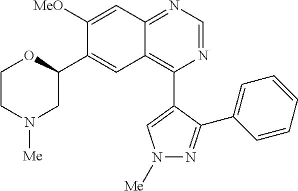 [SMILES: COc1cc2ncnc(-c3cn(C)nc3-c3ccccc3)c2cc1[C@H]1CN(C)CCO1] |
| | or |
| | 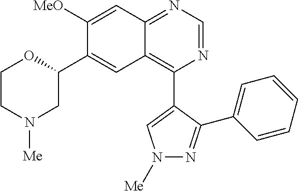 [SMILES: COc1cc2ncnc(-c3cn(C)nc3-c3ccccc3)c2cc1[C@@H]1CN(C)CCO1] |
| 130 | 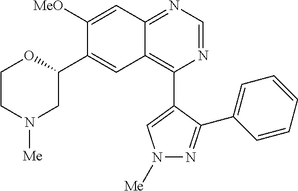 [SMILES: COc1cc2ncnc(-c3cn(C)nc3-c3ccccc3)c2cc1[C@@H]1CN(C)CCO1] |
| | or |
| | 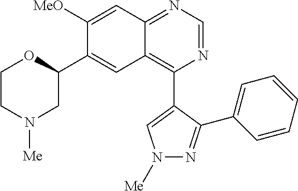 [SMILES: COc1cc2ncnc(-c3cn(C)nc3-c3ccccc3)c2cc1[C@H]1CN(C)CCO1] |
| 131 | 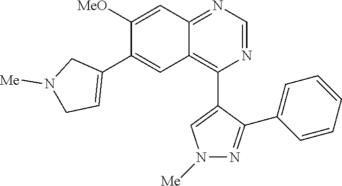 [SMILES: COc1cc2ncnc(-c3cn(C)nc3-c3ccccc3)c2cc1C1=CCN(C)C1] |
| 132 | 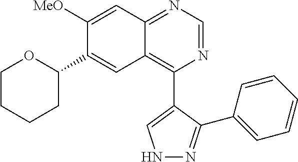 [SMILES: COc1cc2ncnc(-c3c[nH]nc3-c3ccccc3)c2cc1[C@@H]1CCCCO1] |
| | or |
| | 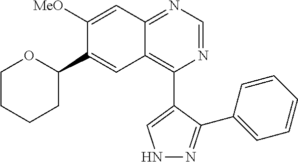 [SMILES: COc1cc2ncnc(-c3c[nH]nc3-c3ccccc3)c2cc1[C@H]1CCCCO1] |
| 133 | 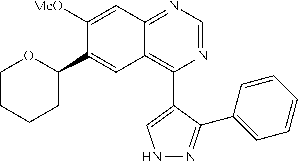 [SMILES: COc1cc2ncnc(-c3c[nH]nc3-c3ccccc3)c2cc1[C@H]1CCCCO1] |
| | or |
| | 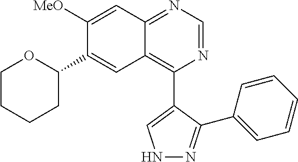 [SMILES: COc1cc2ncnc(-c3c[nH]nc3-c3ccccc3)c2cc1[C@@H]1CCCCO1] |
| 134 | 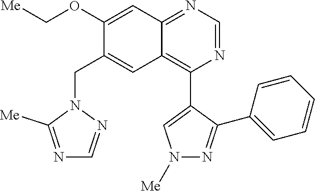 [SMILES: CCOc1cc2ncnc(-c3cn(C)nc3-c3ccccc3)c2cc1Cn1ncnc1C] |
| 135 | 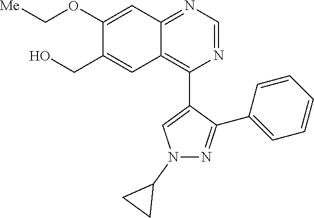 [SMILES: CCOc1cc2ncnc(-c3cn(C4CC4)nc3-c3ccccc3)c2cc1CO] |
| 136 | 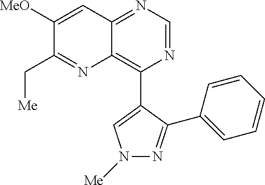 [SMILES: CCc1nc2c(-c3cn(C)nc3-c3ccccc3)ncnc2cc1OC] |
| 137 | 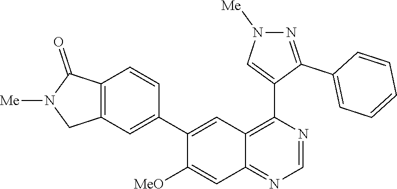 [SMILES: COc1cc2ncnc(-c3cn(C)nc3-c3ccccc3)c2cc1-c1ccc2c(c1)CN(C)C2=O] |
| 138 | 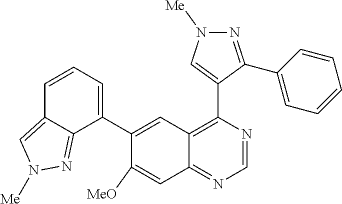 [SMILES: COc1cc2ncnc(-c3cn(C)nc3-c3ccccc3)c2cc1-c1cccc2cn(C)nc12] |
| 139 | 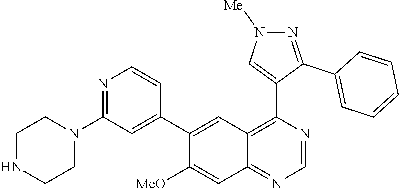 [SMILES: COc1cc2ncnc(-c3cn(C)nc3-c3ccccc3)c2cc1-c1ccnc(N2CCNCC2)c1] |
| 140 | 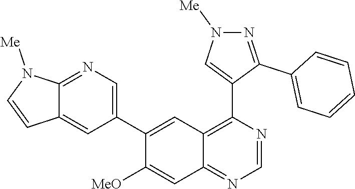 [SMILES: COc1cc2ncnc(-c3cn(C)nc3-c3ccccc3)c2cc1-c1cnc2c(ccn2C)c1] |
| 141 | 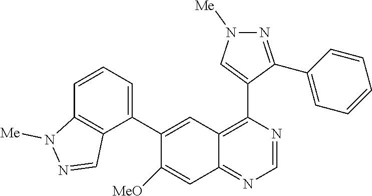 [SMILES: COc1cc2ncnc(-c3cn(C)nc3-c3ccccc3)c2cc1-c1cccc2c1cnn2C] |
| 142 | 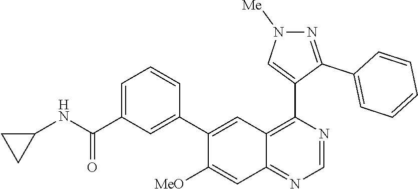 [SMILES: COc1cc2ncnc(-c3cn(C)nc3-c3ccccc3)c2cc1-c1cccc(C(=O)NC2CC2)c1] |
| 143 | 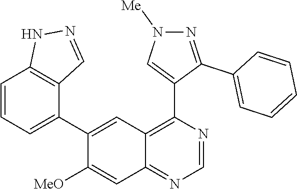 [SMILES: COc1cc2ncnc(-c3cn(C)nc3-c3ccccc3)c2cc1-c1cccc2[nH]ncc12] |
| 144 | 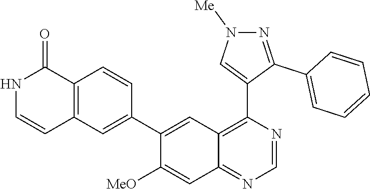 [SMILES: COc1cc2ncnc(-c3cn(C)nc3-c3ccccc3)c2cc1-c1ccc2c(=O)[nH]ccc2c1] |
| 145 | 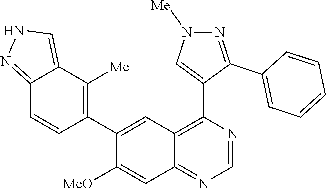 [SMILES: COc1cc2ncnc(-c3cn(C)nc3-c3ccccc3)c2cc1-c1ccc2n[nH]cc2c1C] |
| 146 | 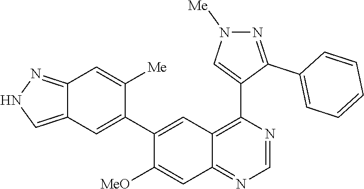 [SMILES: COc1cc2ncnc(-c3cn(C)nc3-c3ccccc3)c2cc1-c1cc2c[nH]nc2cc1C] |
| 147 | 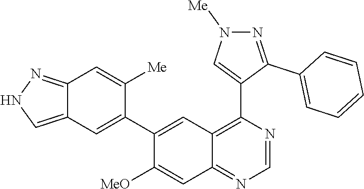 [SMILES: COc1cc2ncnc(-c3cn(C)nc3-c3ccccc3)c2cc1-c1cc2c[nH]nc2cc1C] |
| 148 | 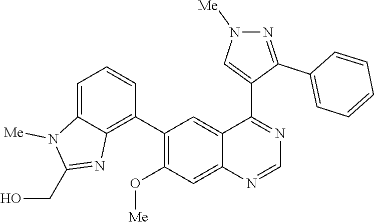 [SMILES: COc1cc2ncnc(-c3cn(C)nc3-c3ccccc3)c2cc1-c1cccc2c1nc(CO)n2C] |
| 149 | 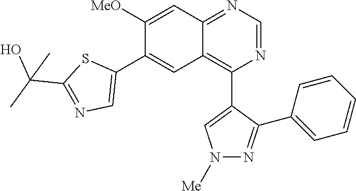 [SMILES: COc1cc2ncnc(-c3cn(C)nc3-c3ccccc3)c2cc1-c1cnc(C(C)(C)O)s1] |
| 150 | 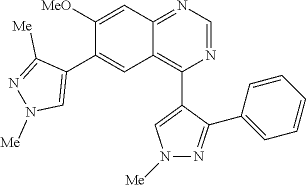 [SMILES: COc1cc2ncnc(-c3cn(C)nc3-c3ccccc3)c2cc1-c1cn(C)nc1C] |
| 151 | 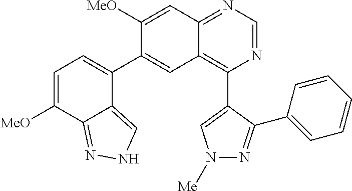 [SMILES: COc1cc2ncnc(-c3cn(C)nc3-c3ccccc3)c2cc1-c1ccc(OC)c2n[nH]cc12] |
| 152 | 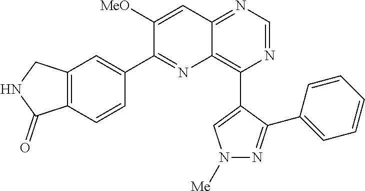 [SMILES: COc1cc2ncnc(-c3cn(C)nc3-c3ccccc3)c2nc1-c1ccc2c(c1)CNC2=O] |
| 153 | 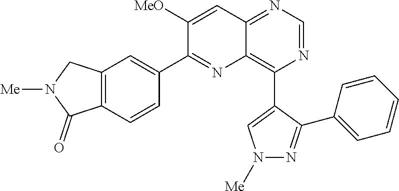 [SMILES: COc1cc2ncnc(-c3cn(C)nc3-c3ccccc3)c2nc1-c1ccc2c(c1)CN(C)C2=O] |
| 154 | 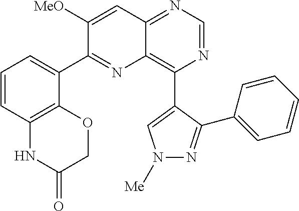 [SMILES: COc1cc2ncnc(-c3cn(C)nc3-c3ccccc3)c2nc1-c1cccc2c1OCC(=O)N2] |
| 155 | 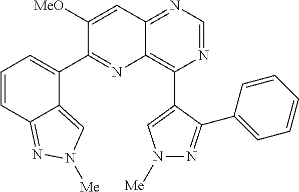 [SMILES: COc1cc2ncnc(-c3cn(C)nc3-c3ccccc3)c2nc1-c1cccc2nn(C)cc12] |
| 156 | 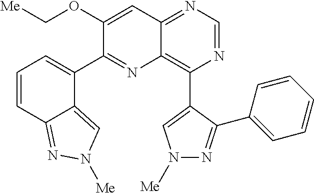 [SMILES: CCOc1cc2ncnc(-c3cn(C)nc3-c3ccccc3)c2nc1-c1cccc2nn(C)cc12] |
| 157 | 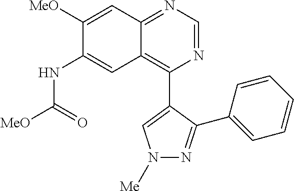 [SMILES: COC(=O)Nc1cc2c(-c3cn(C)nc3-c3ccccc3)ncnc2cc1OC] |
| 158 | 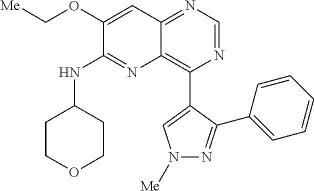 [SMILES: CCOc1cc2ncnc(-c3cn(C)nc3-c3ccccc3)c2nc1NC1CCOCC1] |
| 159 | 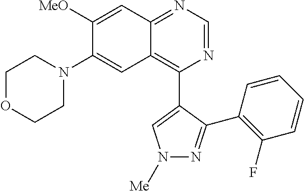 [SMILES: COc1cc2ncnc(-c3cn(C)nc3-c3ccccc3F)c2cc1N1CCOCC1] |
| 160 | 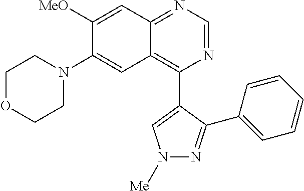 [SMILES: COc1cc2ncnc(-c3cn(C)nc3-c3ccccc3)c2cc1N1CCOCC1] |
| 161 | 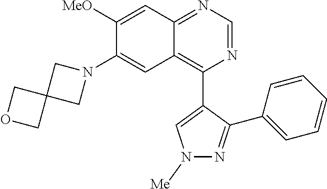 [SMILES: COc1cc2ncnc(-c3cn(C)nc3-c3ccccc3)c2cc1N1CC2(COC2)C1] |
| 162 | 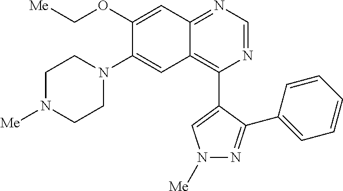 [SMILES: CCOc1cc2ncnc(-c3cn(C)nc3-c3ccccc3)c2cc1N1CCN(C)CC1] |
| 163 | 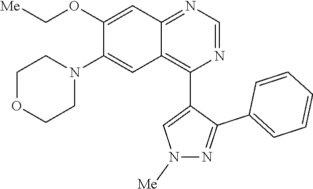 [SMILES: CCOc1cc2ncnc(-c3cn(C)nc3-c3ccccc3)c2cc1N1CCOCC1] |
| 164 | 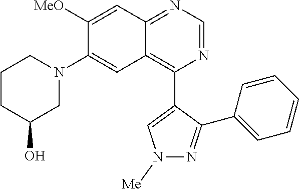 [SMILES: COc1cc2ncnc(-c3cn(C)nc3-c3ccccc3)c2cc1N1CCC[C@H](O)C1] |
| 165 | 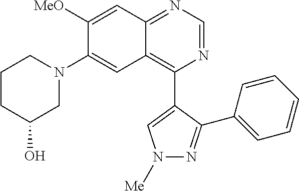 [SMILES: COc1cc2ncnc(-c3cn(C)nc3-c3ccccc3)c2cc1N1CCC[C@@H](O)C1] |
| 166 | 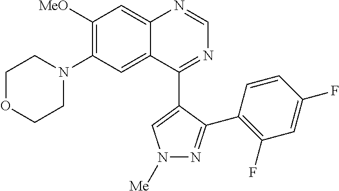 [SMILES: COc1cc2ncnc(-c3cn(C)nc3-c3ccc(F)cc3F)c2cc1N1CCOCC1] |
| 167 | 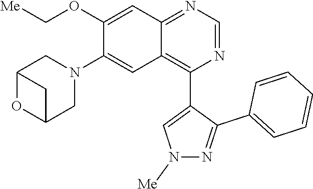 [SMILES: CCOc1cc2ncnc(-c3cn(C)nc3-c3ccccc3)c2cc1N1CC2CC(C1)O2] |
| 168 | 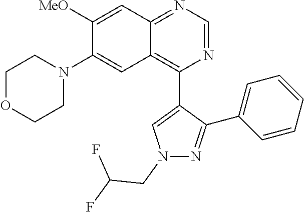 [SMILES: COc1cc2ncnc(-c3cn(CC(F)F)nc3-c3ccccc3)c2cc1N1CCOCC1] |
| 169 | 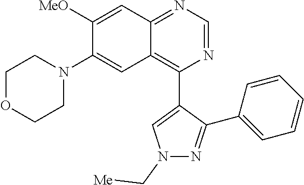 [SMILES: CCn1cc(-c2ncnc3cc(OC)c(N4CCOCC4)cc23)c(-c2ccccc2)n1] |
| 170 | 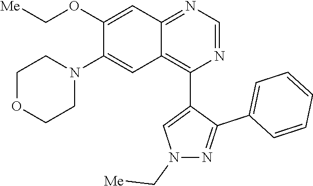 [SMILES: CCOc1cc2ncnc(-c3cn(CC)nc3-c3ccccc3)c2cc1N1CCOCC1] |
| 171 | 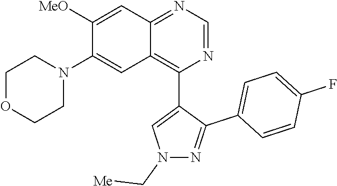 [SMILES: CCn1cc(-c2ncnc3cc(OC)c(N4CCOCC4)cc23)c(-c2ccc(F)cc2)n1] |
| 172 | 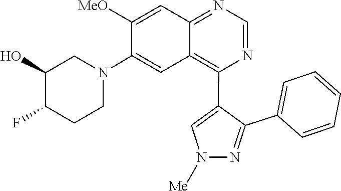 [SMILES: COc1cc2ncnc(-c3cn(C)nc3-c3ccccc3)c2cc1N1CC[C@H](F)[C@@H](O)C1] |
| | or |
| | 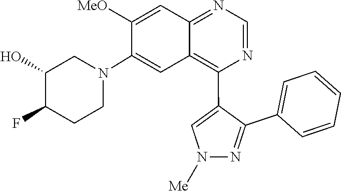 [SMILES: COc1cc2ncnc(-c3cn(C)nc3-c3ccccc3)c2cc1N1CC[C@@H](F)[C@H](O)C1] |
| 173 | 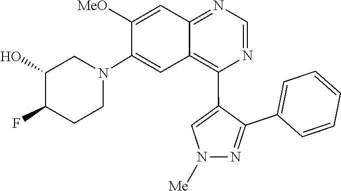 [SMILES: COc1cc2ncnc(-c3cn(C)nc3-c3ccccc3)c2cc1N1CC[C@@H](F)[C@H](O)C1] |
| | or |
| | 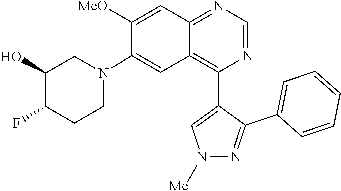 [SMILES: COc1cc2ncnc(-c3cn(C)nc3-c3ccccc3)c2cc1N1CC[C@H](F)[C@@H](O)C1] |
| 174 | 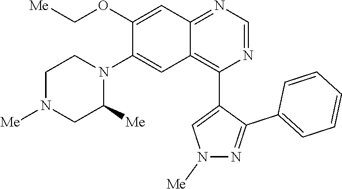 [SMILES: CCOc1cc2ncnc(-c3cn(C)nc3-c3ccccc3)c2cc1N1CCN(C)C[C@@H]1C] |
| 175 | 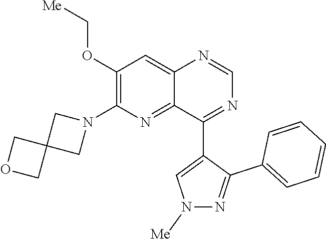 [SMILES: CCOc1cc2ncnc(-c3cn(C)nc3-c3ccccc3)c2nc1N1CC2(COC2)C1] |
| 176 | 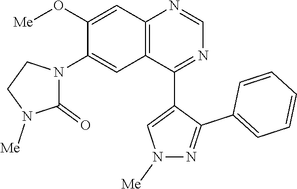 [SMILES: COc1cc2ncnc(-c3cn(C)nc3-c3ccccc3)c2cc1N1CCN(C)C1=O] |
| 177 | 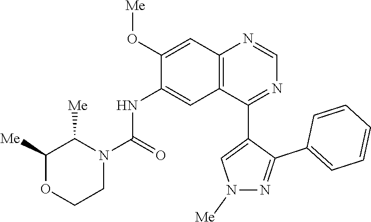 [SMILES: COc1cc2ncnc(-c3cn(C)nc3-c3ccccc3)c2cc1NC(=O)N1CCO[C@@H](C)[C@@H]1C] |
| 178 | 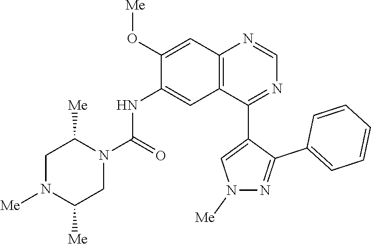 [SMILES: COc1cc2ncnc(-c3cn(C)nc3-c3ccccc3)c2cc1NC(=O)N1C[C@H](C)N(C)C[C@@H]1C] |
| 179 | 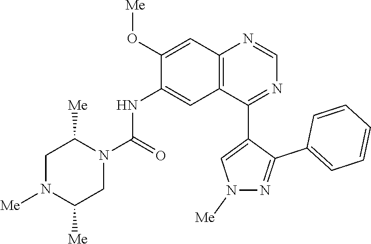 [SMILES: COc1cc2ncnc(-c3cn(C)nc3-c3ccccc3)c2cc1NC(=O)N1C[C@H](C)N(C)C[C@@H]1C] |
| 180 | 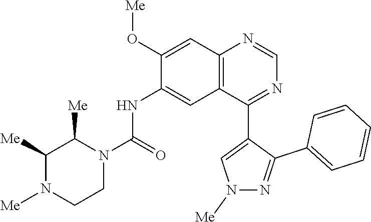 [SMILES: COc1cc2ncnc(-c3cn(C)nc3-c3ccccc3)c2cc1NC(=O)N1CCN(C)[C@@H](C)[C@H]1C] |
| | or |
| | 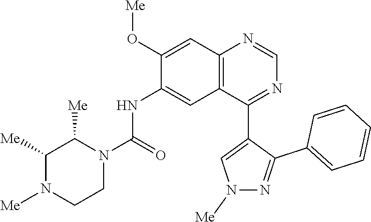 [SMILES: COc1cc2ncnc(-c3cn(C)nc3-c3ccccc3)c2cc1NC(=O)N1CCN(C)[C@H](C)[C@@H]1C] |
| 181 | 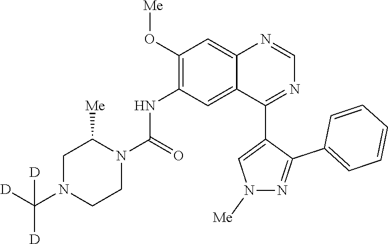 [SMILES: [2H]C([2H])([2H])N1CCN(C(=O)Nc2cc3c(-c4cn(C)nc4-c4ccccc4)ncnc3cc2OC)[C@@H](C)C1] |
| 182 | 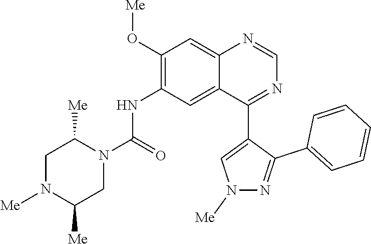 [SMILES: COc1cc2ncnc(-c3cn(C)nc3-c3ccccc3)c2cc1NC(=O)N1C[C@@H](C)N(C)C[C@@H]1C] |
| 183 | 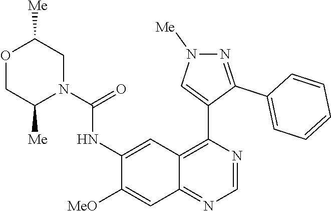 [SMILES: COc1cc2ncnc(-c3cn(C)nc3-c3ccccc3)c2cc1NC(=O)N1C[C@@H](C)OC[C@@H]1C] |
| 184 | 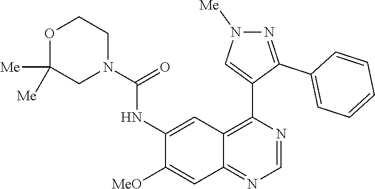 [SMILES: COc1cc2ncnc(-c3cn(C)nc3-c3ccccc3)c2cc1NC(=O)N1CCOC(C)(C)C1] |
| 185 | 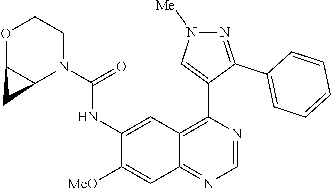 [SMILES: COc1cc2ncnc(-c3cn(C)nc3-c3ccccc3)c2cc1NC(=O)N1CCO[C@@H]2C[C@@H]21] |
| 186 | 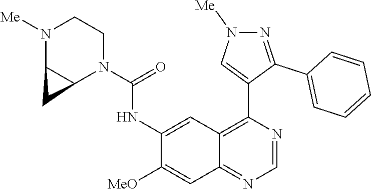 [SMILES: COc1cc2ncnc(-c3cn(C)nc3-c3ccccc3)c2cc1NC(=O)N1CCN(C)[C@@H]2C[C@@H]21] |
| 187 | 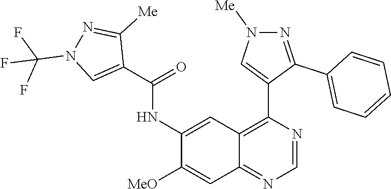 [SMILES: COc1cc2ncnc(-c3cn(C)nc3-c3ccccc3)c2cc1NC(=O)c1cn(C(F)(F)F)nc1C] |
| 188 | 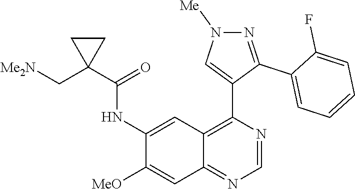 [SMILES: COc1cc2ncnc(-c3cn(C)nc3-c3ccccc3F)c2cc1NC(=O)C1(CN(C)C)CC1] |
| 189 | 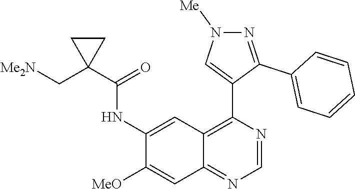 [SMILES: COc1cc2ncnc(-c3cn(C)nc3-c3ccccc3)c2cc1NC(=O)C1(CN(C)C)CC1] |
| 190 | 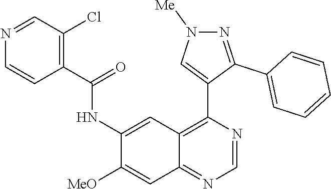 [SMILES: COc1cc2ncnc(-c3cn(C)nc3-c3ccccc3)c2cc1NC(=O)c1ccncc1Cl] |
| 191 | 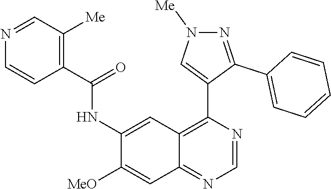 [SMILES: COc1cc2ncnc(-c3cn(C)nc3-c3ccccc3)c2cc1NC(=O)c1ccncc1C] |
| 192 | 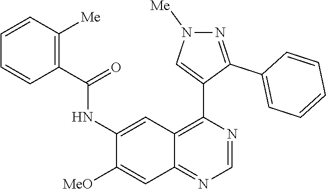 [SMILES: COc1cc2ncnc(-c3cn(C)nc3-c3ccccc3)c2cc1NC(=O)c1ccccc1C] |
| 193 | 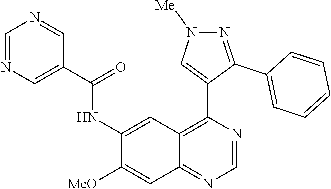 [SMILES: COc1cc2ncnc(-c3cn(C)nc3-c3ccccc3)c2cc1NC(=O)c1cncnc1] |
| 194 | 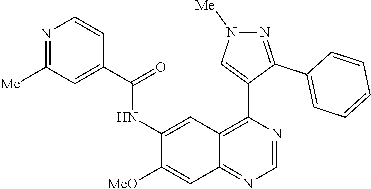 [SMILES: COc1cc2ncnc(-c3cn(C)nc3-c3ccccc3)c2cc1NC(=O)c1ccnc(C)c1] |
| 195 | 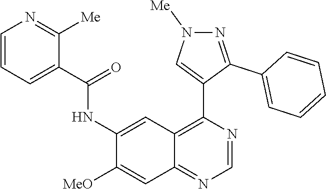 [SMILES: COc1cc2ncnc(-c3cn(C)nc3-c3ccccc3)c2cc1NC(=O)c1cccnc1C] |
| 196 | 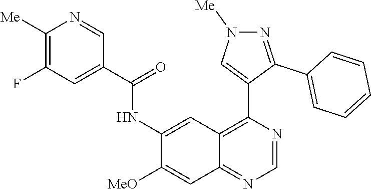 [SMILES: COc1cc2ncnc(-c3cn(C)nc3-c3ccccc3)c2cc1NC(=O)c1cnc(C)c(F)c1] |
| 197 | 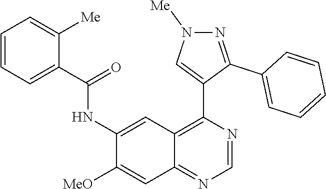 [SMILES: COc1cc2ncnc(-c3cn(C)nc3-c3ccccc3)c2cc1NC(=O)c1ccccc1C] |
| 198 | 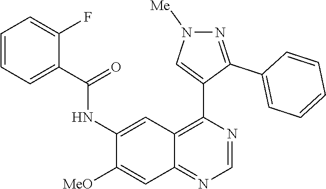 [SMILES: COc1cc2ncnc(-c3cn(C)nc3-c3ccccc3)c2cc1NC(=O)c1ccccc1F] |
| 199 | 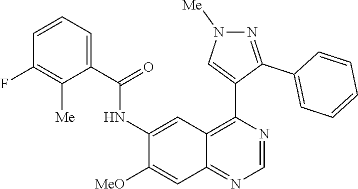 [SMILES: COc1cc2ncnc(-c3cn(C)nc3-c3ccccc3)c2cc1NC(=O)c1cccc(F)c1C] |
| 200 | 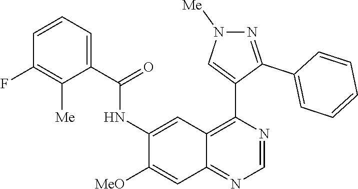 [SMILES: COc1cc2ncnc(-c3cn(C)nc3-c3ccccc3)c2cc1NC(=O)c1cccc(F)c1C] |
| 201 | 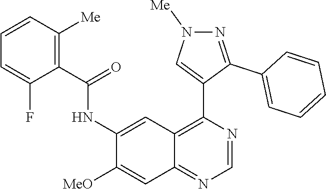 [SMILES: COc1cc2ncnc(-c3cn(C)nc3-c3ccccc3)c2cc1NC(=O)c1c(C)cccc1F] |
| 202 | 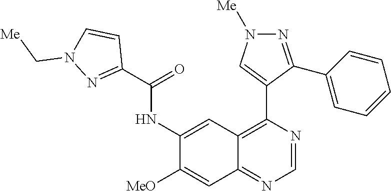 [SMILES: CCn1ccc(C(=O)Nc2cc3c(-c4cn(C)nc4-c4ccccc4)ncnc3cc2OC)n1] |
| 203 | 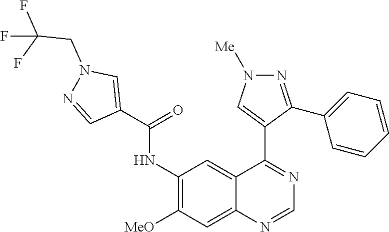 [SMILES: COc1cc2ncnc(-c3cn(C)nc3-c3ccccc3)c2cc1NC(=O)c1cnn(CC(F)(F)F)c1] |
| 204 | 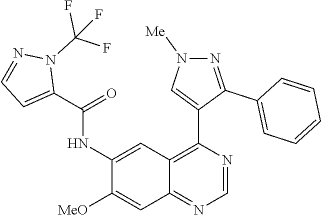 [SMILES: COc1cc2ncnc(-c3cn(C)nc3-c3ccccc3)c2cc1NC(=O)c1ccnn1C(F)(F)F] |
| 205 | 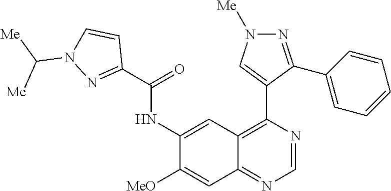 [SMILES: COc1cc2ncnc(-c3cn(C)nc3-c3ccccc3)c2cc1NC(=O)c1ccn(C(C)C)n1] |
| 206 | 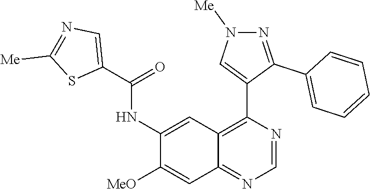 [SMILES: COc1cc2ncnc(-c3cn(C)nc3-c3ccccc3)c2cc1NC(=O)c1cnc(C)s1] |
| 207 | 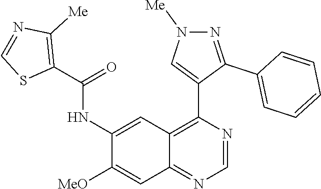 [SMILES: COc1cc2ncnc(-c3cn(C)nc3-c3ccccc3)c2cc1NC(=O)c1scnc1C] |
| 208 | 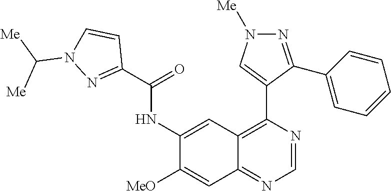 [SMILES: COc1cc2ncnc(-c3cn(C)nc3-c3ccccc3)c2cc1NC(=O)c1ccn(C(C)C)n1] |
| 209 | 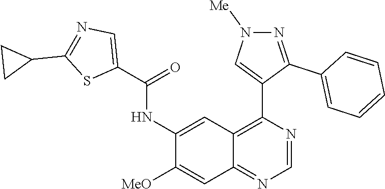 [SMILES: COc1cc2ncnc(-c3cn(C)nc3-c3ccccc3)c2cc1NC(=O)c1cnc(C2CC2)s1] |
| 210 | 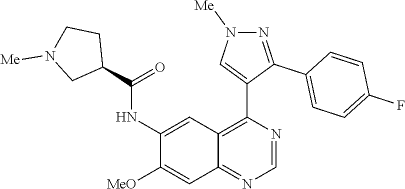 [SMILES: COc1cc2ncnc(-c3cn(C)nc3-c3ccc(F)cc3)c2cc1NC(=O)[C@@H]1CCN(C)C1] |
| 211 | 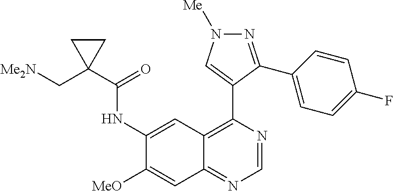 [SMILES: COc1cc2ncnc(-c3cn(C)nc3-c3ccc(F)cc3)c2cc1NC(=O)C1(CN(C)C)CC1] |
| 212 | 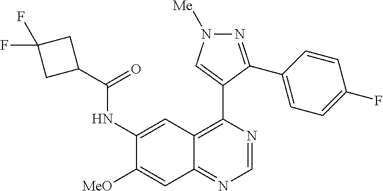 [SMILES: COc1cc2ncnc(-c3cn(C)nc3-c3ccc(F)cc3)c2cc1NC(=O)C1CC(F)(F)C1] |
| 213 | 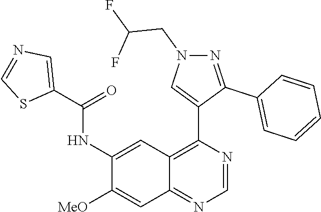 [SMILES: COc1cc2ncnc(-c3cn(CC(F)F)nc3-c3ccccc3)c2cc1NC(=O)c1cncs1] |
| 214 | 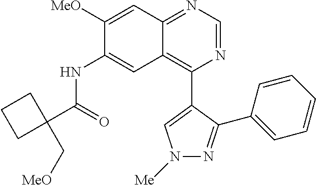 [SMILES: COCC1(C(=O)Nc2cc3c(-c4cn(C)nc4-c4ccccc4)ncnc3cc2OC)CCC1] |
| 215 | 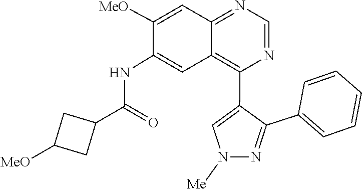 [SMILES: COc1cc2ncnc(-c3cn(C)nc3-c3ccccc3)c2cc1NC(=O)C1CC(OC)C1] |
| 216 | 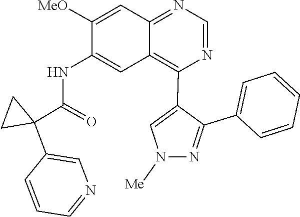 [SMILES: COc1cc2ncnc(-c3cn(C)nc3-c3ccccc3)c2cc1NC(=O)C1(c2cccnc2)CC1] |
| 217 | 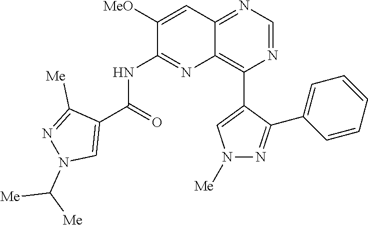 [SMILES: COc1cc2ncnc(-c3cn(C)nc3-c3ccccc3)c2nc1NC(=O)c1cn(C(C)C)nc1C] |
| 218 | 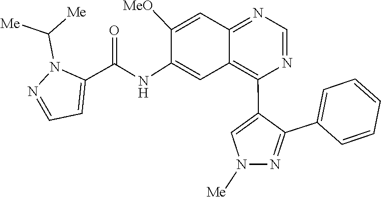 [SMILES: COc1cc2ncnc(-c3cn(C)nc3-c3ccccc3)c2cc1NC(=O)c1ccnn1C(C)C] |
| 219 | 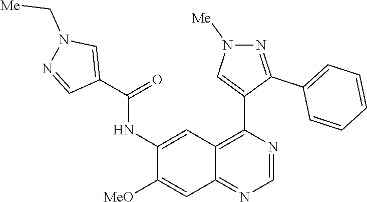 [SMILES: CCn1cc(C(=O)Nc2cc3c(-c4cn(C)nc4-c4ccccc4)ncnc3cc2OC)cn1] |
| 220 | 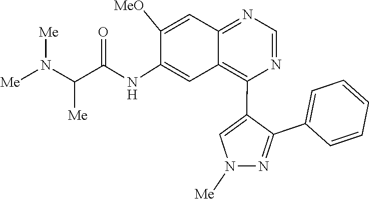 [SMILES: COc1cc2ncnc(-c3cn(C)nc3-c3ccccc3)c2cc1NC(=O)C(C)N(C)C] |
| 221 | 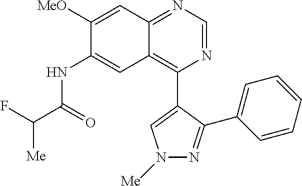 [SMILES: COc1cc2ncnc(-c3cn(C)nc3-c3ccccc3)c2cc1NC(=O)C(C)F] |
| 222 | 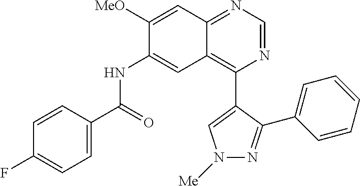 [SMILES: COc1cc2ncnc(-c3cn(C)nc3-c3ccccc3)c2cc1NC(=O)c1ccc(F)cc1] |
| 223 | 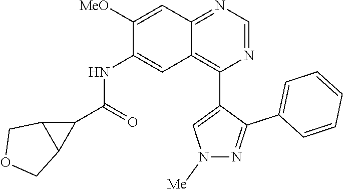 [SMILES: COc1cc2ncnc(-c3cn(C)nc3-c3ccccc3)c2cc1NC(=O)C1C2COCC21] |
| 224 | 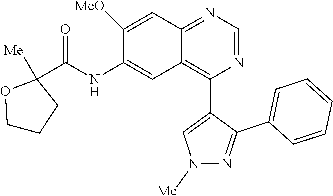 [SMILES: COc1cc2ncnc(-c3cn(C)nc3-c3ccccc3)c2cc1NC(=O)C1(C)CCCO1] |
| 225 | 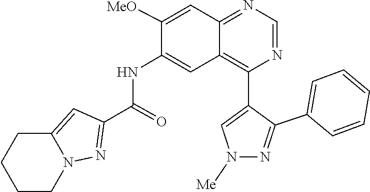 [SMILES: COc1cc2ncnc(-c3cn(C)nc3-c3ccccc3)c2cc1NC(=O)c1cc2n(n1)CCCC2] |
| 226 | 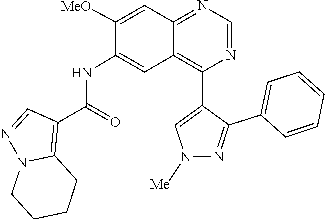 [SMILES: COc1cc2ncnc(-c3cn(C)nc3-c3ccccc3)c2cc1NC(=O)c1cnn2c1CCCC2] |
| 227 | 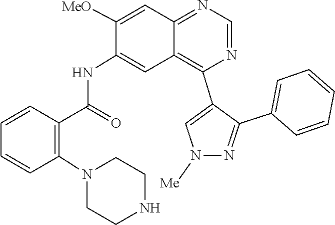 [SMILES: COc1cc2ncnc(-c3cn(C)nc3-c3ccccc3)c2cc1NC(=O)c1ccccc1N1CCNCC1] |
| 228 | 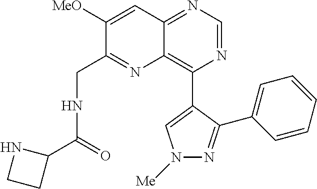 [SMILES: COc1cc2ncnc(-c3cn(C)nc3-c3ccccc3)c2nc1CNC(=O)C1CCN1] |
| 229 | 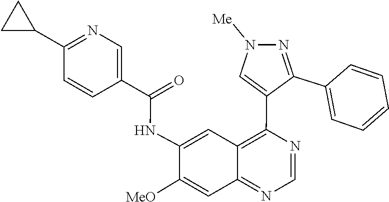 [SMILES: COc1cc2ncnc(-c3cn(C)nc3-c3ccccc3)c2cc1NC(=O)c1ccc(C2CC2)nc1] |
| 230 | 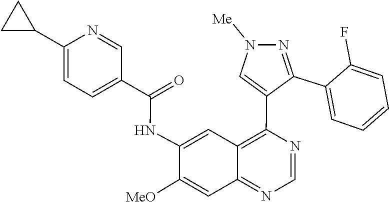 [SMILES: COc1cc2ncnc(-c3cn(C)nc3-c3ccccc3F)c2cc1NC(=O)c1ccc(C2CC2)nc1] |
| 231 | 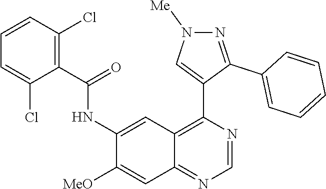 [SMILES: COc1cc2ncnc(-c3cn(C)nc3-c3ccccc3)c2cc1NC(=O)c1c(Cl)cccc1Cl] |
| 232 | 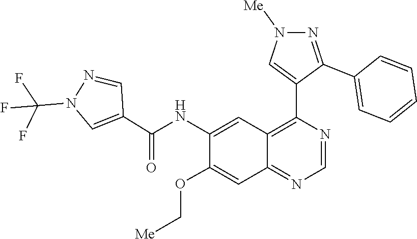 [SMILES: CCOc1cc2ncnc(-c3cn(C)nc3-c3ccccc3)c2cc1NC(=O)c1cnn(C(F)(F)F)c1] |
| 233 | 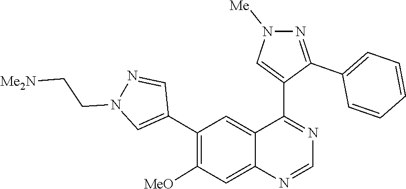 [SMILES: COc1cc2ncnc(-c3cn(C)nc3-c3ccccc3)c2cc1-c1cnn(CCN(C)C)c1] |
| 234 | 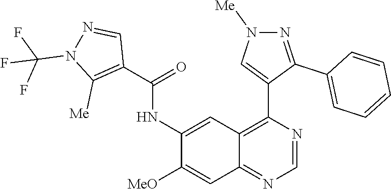 [SMILES: COc1cc2ncnc(-c3cn(C)nc3-c3ccccc3)c2cc1NC(=O)c1cnn(C(F)(F)F)c1C] |
| 235 | 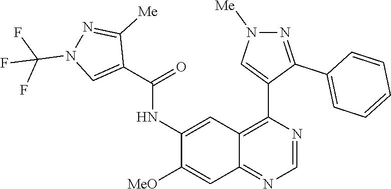 [SMILES: COc1cc2ncnc(-c3cn(C)nc3-c3ccccc3)c2cc1NC(=O)c1cn(C(F)(F)F)nc1C] |
| 236 | 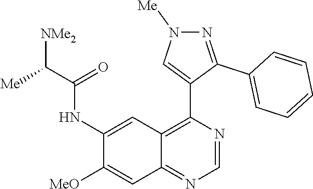 [SMILES: COc1cc2ncnc(-c3cn(C)nc3-c3ccccc3)c2cc1NC(=O)[C@H](C)N(C)C] |
| | or |
| | 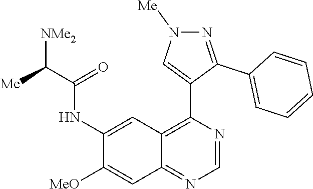 [SMILES: COc1cc2ncnc(-c3cn(C)nc3-c3ccccc3)c2cc1NC(=O)[C@@H](C)N(C)C] |
| 237 | 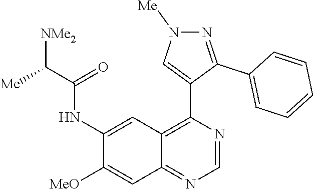 [SMILES: COc1cc2ncnc(-c3cn(C)nc3-c3ccccc3)c2cc1NC(=O)[C@H](C)N(C)C] |
| | or |
| | 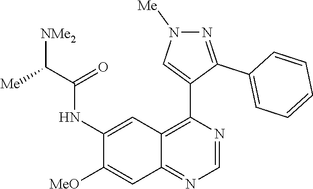 [SMILES: COc1cc2ncnc(-c3cn(C)nc3-c3ccccc3)c2cc1NC(=O)[C@H](C)N(C)C] |
| 238 | 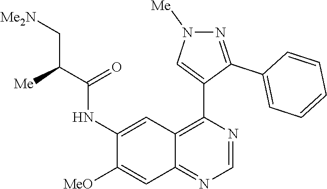 [SMILES: COc1cc2ncnc(-c3cn(C)nc3-c3ccccc3)c2cc1NC(=O)[C@@H](C)CN(C)C] |
| | or |
| | 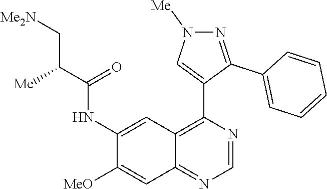 [SMILES: COc1cc2ncnc(-c3cn(C)nc3-c3ccccc3)c2cc1NC(=O)[C@H](C)CN(C)C] |
| 239 | 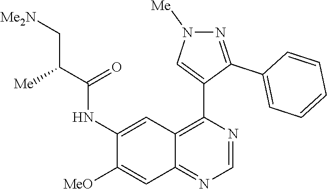 [SMILES: COc1cc2ncnc(-c3cn(C)nc3-c3ccccc3)c2cc1NC(=O)[C@H](C)CN(C)C] |
| | or |
| | 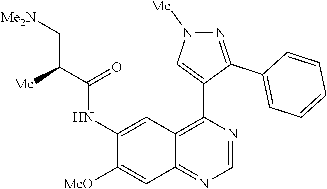 [SMILES: COc1cc2ncnc(-c3cn(C)nc3-c3ccccc3)c2cc1NC(=O)[C@@H](C)CN(C)C] |
| 240 | 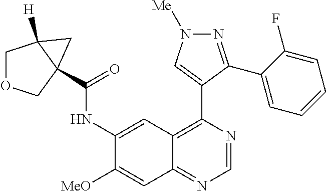 [SMILES: COc1cc2ncnc(-c3cn(C)nc3-c3ccccc3F)c2cc1NC(=O)[C@@]12COC[C@@H]1C2] |
| | or |
| | 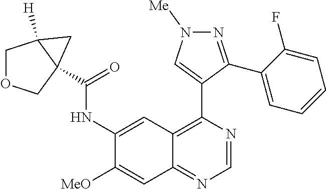 [SMILES: COc1cc2ncnc(-c3cn(C)nc3-c3ccccc3F)c2cc1NC(=O)[C@]12COC[C@H]1C2] |
| 241 | 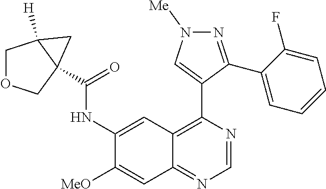 [SMILES: COc1cc2ncnc(-c3cn(C)nc3-c3ccccc3F)c2cc1NC(=O)[C@]12COC[C@H]1C2] |
| | or |
| | 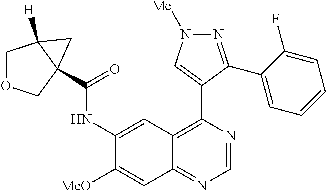 [SMILES: COc1cc2ncnc(-c3cn(C)nc3-c3ccccc3F)c2cc1NC(=O)[C@@]12COC[C@@H]1C2] |
| 242 | 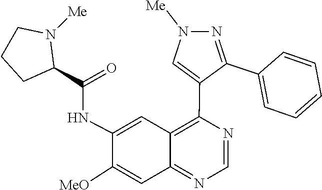 [SMILES: COc1cc2ncnc(-c3cn(C)nc3-c3ccccc3)c2cc1NC(=O)[C@H]1CCCN1C] |
| | or |
| | 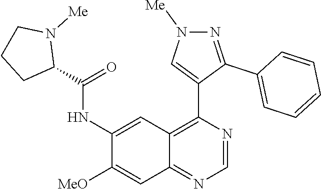 [SMILES: COc1cc2ncnc(-c3cn(C)nc3-c3ccccc3)c2cc1NC(=O)[C@@H]1CCCN1C] |
| 243 | 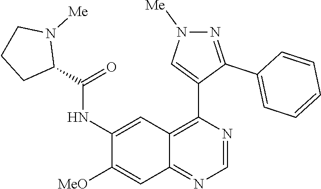 [SMILES: COc1cc2ncnc(-c3cn(C)nc3-c3ccccc3)c2cc1NC(=O)[C@@H]1CCCN1C] |
| | or |
| | 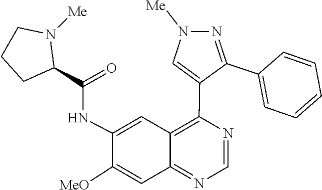 [SMILES: COc1cc2ncnc(-c3cn(C)nc3-c3ccccc3)c2cc1NC(=O)[C@H]1CCCN1C] |
| 244 | 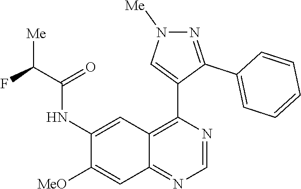 [SMILES: COc1cc2ncnc(-c3cn(C)nc3-c3ccccc3)c2cc1NC(=O)[C@H](C)F] |
| | or |
| | 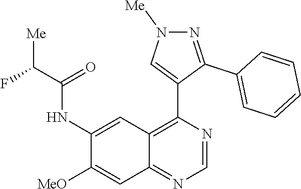 [SMILES: COc1cc2ncnc(-c3cn(C)nc3-c3ccccc3)c2cc1NC(=O)[C@@H](C)F] |
| 245 | 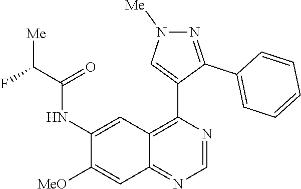 [SMILES: COc1cc2ncnc(-c3cn(C)nc3-c3ccccc3)c2cc1NC(=O)[C@@H](C)F] |
| | or |
| | 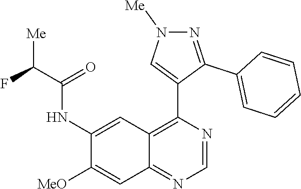 [SMILES: COc1cc2ncnc(-c3cn(C)nc3-c3ccccc3)c2cc1NC(=O)[C@H](C)F] |
| 246 | 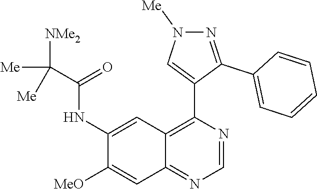 [SMILES: COc1cc2ncnc(-c3cn(C)nc3-c3ccccc3)c2cc1NC(=O)C(C)(C)N(C)C] |
| 247 | 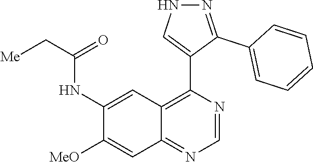 [SMILES: CCC(=O)Nc1cc2c(-c3c[nH]nc3-c3ccccc3)ncnc2cc1OC] |
| 248 | 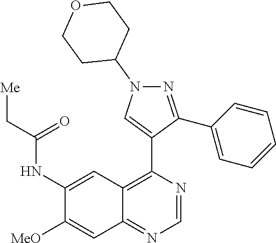 [SMILES: CCC(=O)Nc1cc2c(-c3cn(C4CCOCC4)nc3-c3ccccc3)ncnc2cc1OC] |
| 249 | 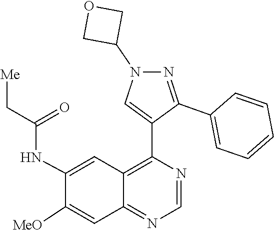 [SMILES: CCC(=O)Nc1cc2c(-c3cn(C4COC4)nc3-c3ccccc3)ncnc2cc1OC] |
| 250 | 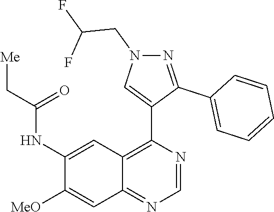 [SMILES: CCC(=O)Nc1cc2c(-c3cn(CC(F)F)nc3-c3ccccc3)ncnc2cc1OC] |
| 251 | 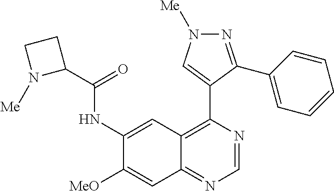 [SMILES: COc1cc2ncnc(-c3cn(C)nc3-c3ccccc3)c2cc1NC(=O)C1CCN1C] |
| 252 | 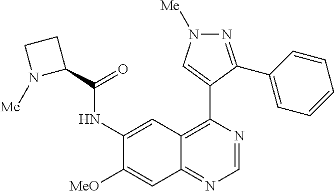 [SMILES: COc1cc2ncnc(-c3cn(C)nc3-c3ccccc3)c2cc1NC(=O)[C@@H]1CCN1C] |
| | or |
| | 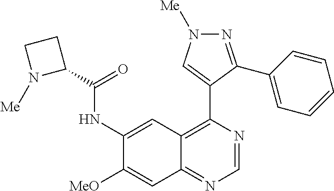 [SMILES: COc1cc2ncnc(-c3cn(C)nc3-c3ccccc3)c2cc1NC(=O)[C@H]1CCN1C] |
| 253 | 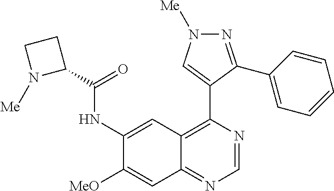 [SMILES: COc1cc2ncnc(-c3cn(C)nc3-c3ccccc3)c2cc1NC(=O)[C@H]1CCN1C] |
| | or |
| | 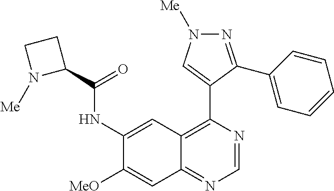 [SMILES: COc1cc2ncnc(-c3cn(C)nc3-c3ccccc3)c2cc1NC(=O)[C@@H]1CCN1C] |
| 254 | 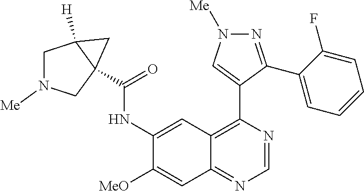 [SMILES: COc1cc2ncnc(-c3cn(C)nc3-c3ccccc3F)c2cc1NC(=O)[C@@]12C[C@@H]1CN(C)C2] |
| | or |
| | 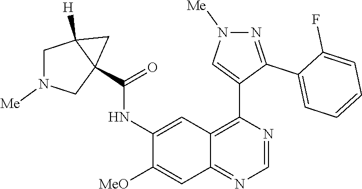 [SMILES: COc1cc2ncnc(-c3cn(C)nc3-c3ccccc3F)c2cc1NC(=O)[C@]12C[C@H]1CN(C)C2] |
| 255 | 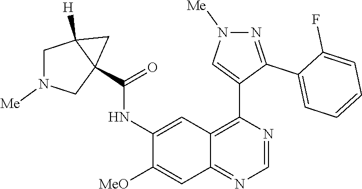 [SMILES: COc1cc2ncnc(-c3cn(C)nc3-c3ccccc3F)c2cc1NC(=O)[C@]12C[C@H]1CN(C)C2] |
| | or |
| | 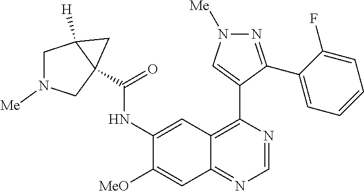 [SMILES: COc1cc2ncnc(-c3cn(C)nc3-c3ccccc3F)c2cc1NC(=O)[C@@]12C[C@@H]1CN(C)C2] |
| 256 | 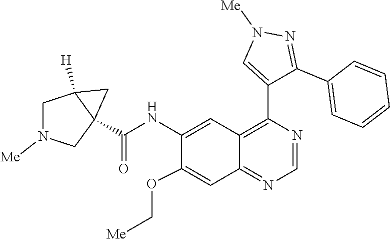 [SMILES: CCOc1cc2ncnc(-c3cn(C)nc3-c3ccccc3)c2cc1NC(=O)[C@@]12C[C@@H]1CN(C)C2] |
| | or |
| | 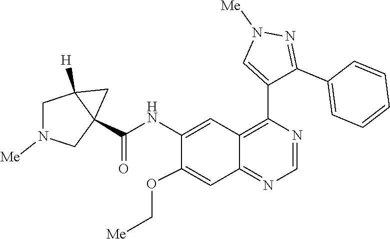 [SMILES: CCOc1cc2ncnc(-c3cn(C)nc3-c3ccccc3)c2cc1NC(=O)[C@]12C[C@H]1CN(C)C2] |
| 257 | 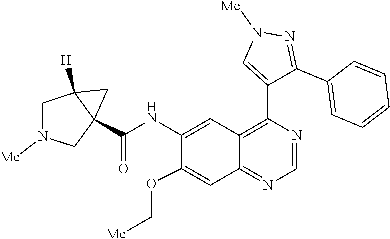 [SMILES: CCOc1cc2ncnc(-c3cn(C)nc3-c3ccccc3)c2cc1NC(=O)[C@]12C[C@H]1CN(C)C2] |
| | or |
| | 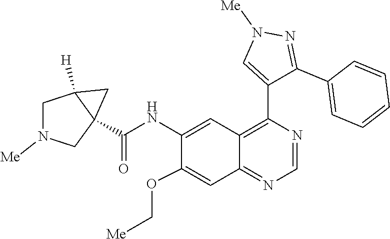 [SMILES: CCOc1cc2ncnc(-c3cn(C)nc3-c3ccccc3)c2cc1NC(=O)[C@@]12C[C@@H]1CN(C)C2] |
| 258 | 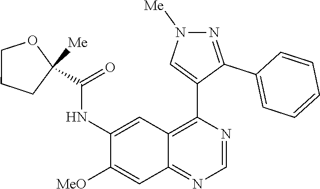 [SMILES: COc1cc2ncnc(-c3cn(C)nc3-c3ccccc3)c2cc1NC(=O)[C@]1(C)CCCO1] |
| | or |
| | 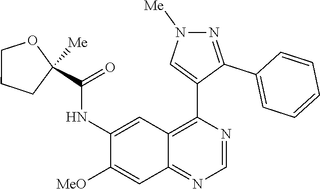 [SMILES: COc1cc2ncnc(-c3cn(C)nc3-c3ccccc3)c2cc1NC(=O)[C@@]1(C)CCCO1] |
| 259 | 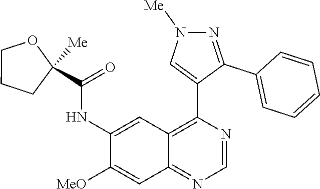 [SMILES: COc1cc2ncnc(-c3cn(C)nc3-c3ccccc3)c2cc1NC(=O)[C@@]1(C)CCCO1] |
| | or |
| | 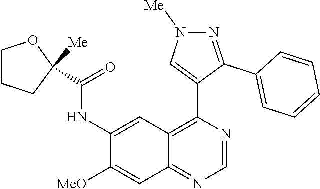 [SMILES: COc1cc2ncnc(-c3cn(C)nc3-c3ccccc3)c2cc1NC(=O)[C@]1(C)CCCO1] |
| 260 | 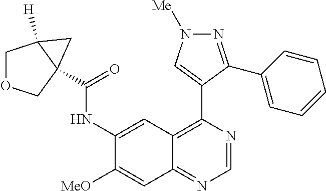 [SMILES: COc1cc2ncnc(-c3cn(C)nc3-c3ccccc3)c2cc1NC(=O)[C@]12COC[C@H]1C2] |
| | or |
| | 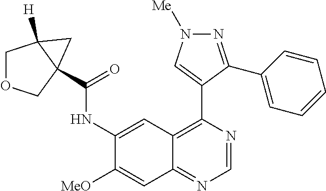 [SMILES: COc1cc2ncnc(-c3cn(C)nc3-c3ccccc3)c2cc1NC(=O)[C@@]12COC[C@@H]1C2] |
| 261 | 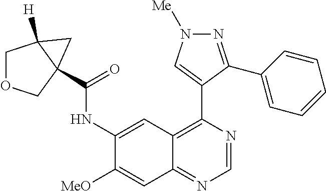 [SMILES: COc1cc2ncnc(-c3cn(C)nc3-c3ccccc3)c2cc1NC(=O)[C@@]12COC[C@@H]1C2] |
| | or |
| | 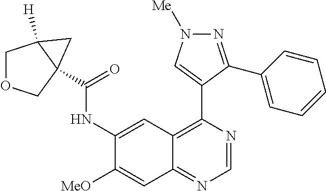 [SMILES: COc1cc2ncnc(-c3cn(C)nc3-c3ccccc3)c2cc1NC(=O)[C@]12COC[C@H]1C2] |
| 262 | 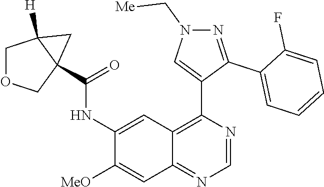 [SMILES: CCn1cc(-c2ncnc3cc(OC)c(NC(=O)[C@@]45COC[C@@H]4C5)cc23)c(-c2ccccc2F)n1] |
| | or |
| | 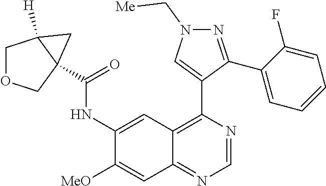 [SMILES: CCn1cc(-c2ncnc3cc(OC)c(NC(=O)[C@]45COC[C@H]4C5)cc23)c(-c2ccccc2F)n1] |
| 263 | 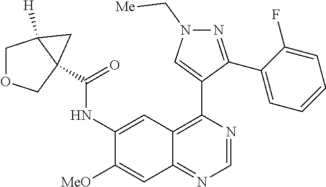 [SMILES: CCn1cc(-c2ncnc3cc(OC)c(NC(=O)[C@]45COC[C@H]4C5)cc23)c(-c2ccccc2F)n1] |
| | or |
| | 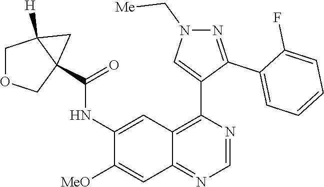 [SMILES: CCn1cc(-c2ncnc3cc(OC)c(NC(=O)[C@@]45COC[C@@H]4C5)cc23)c(-c2ccccc2F)n1] |
| 264 | 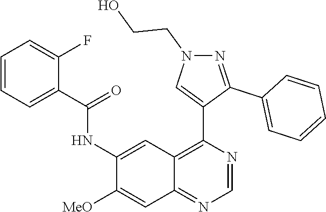 [SMILES: COc1cc2ncnc(-c3cn(CCO)nc3-c3ccccc3)c2cc1NC(=O)c1ccccc1F] |
| 265 | 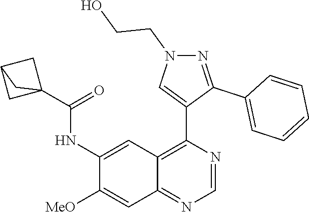 [SMILES: COc1cc2ncnc(-c3cn(CCO)nc3-c3ccccc3)c2cc1NC(=O)C12CC(C1)C2] |
| 266 | 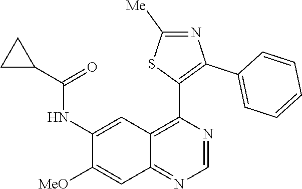 [SMILES: COc1cc2ncnc(-c3sc(C)nc3-c3ccccc3)c2cc1NC(=O)C1CC1] |
| 267 | 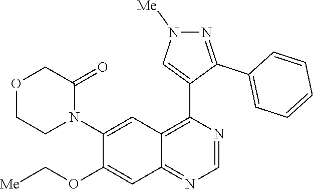 [SMILES: CCOc1cc2ncnc(-c3cn(C)nc3-c3ccccc3)c2cc1N1CCOCC1=O] |
| 268 | 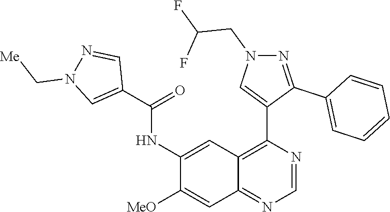 [SMILES: CCn1cc(C(=O)Nc2cc3c(-c4cn(CC(F)F)nc4-c4ccccc4)ncnc3cc2OC)cn1] |
| 269 | 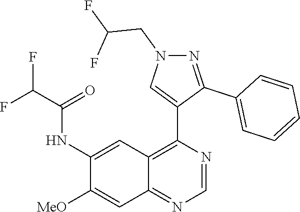 [SMILES: COc1cc2ncnc(-c3cn(CC(F)F)nc3-c3ccccc3)c2cc1NC(=O)C(F)F] |
| 270 | 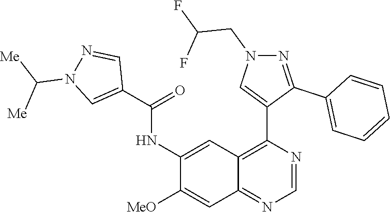 [SMILES: COc1cc2ncnc(-c3cn(CC(F)F)nc3-c3ccccc3)c2cc1NC(=O)c1cnn(C(C)C)c1] |
| 271 | 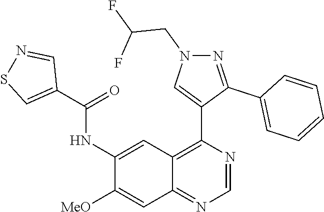 [SMILES: COc1cc2ncnc(-c3cn(CC(F)F)nc3-c3ccccc3)c2cc1NC(=O)c1cnsc1] |
| 272 | 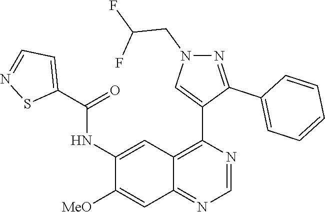 [SMILES: COc1cc2ncnc(-c3cn(CC(F)F)nc3-c3ccccc3)c2cc1NC(=O)c1ccns1] |
| 273 | 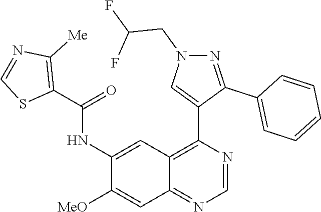 [SMILES: COc1cc2ncnc(-c3cn(CC(F)F)nc3-c3ccccc3)c2cc1NC(=O)c1scnc1C] |
| 274 | 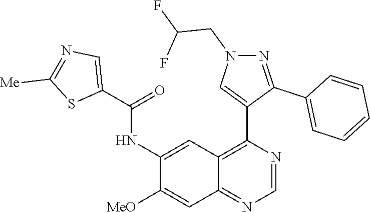 [SMILES: COc1cc2ncnc(-c3cn(CC(F)F)nc3-c3ccccc3)c2cc1NC(=O)c1cnc(C)s1] |
| 275 | 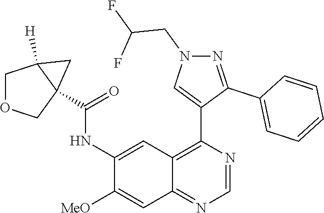 [SMILES: COc1cc2ncnc(-c3cn(CC(F)F)nc3-c3ccccc3)c2cc1NC(=O)[C@]12COC[C@H]1C2] |
| | or |
| | 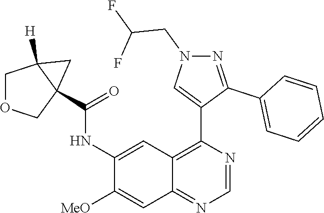 [SMILES: COc1cc2ncnc(-c3cn(CC(F)F)nc3-c3ccccc3)c2cc1NC(=O)[C@@]12COC[C@@H]1C2] |
| 276 | 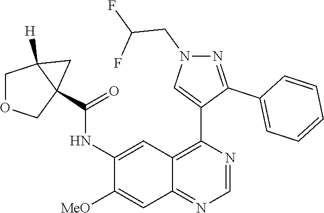 [SMILES: COc1cc2ncnc(-c3cn(CC(F)F)nc3-c3ccccc3)c2cc1NC(=O)[C@@]12COC[C@@H]1C2] |
| | or |
| | 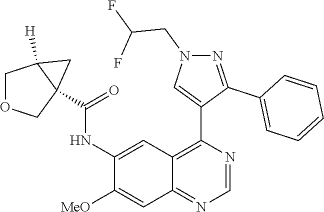 [SMILES: COc1cc2ncnc(-c3cn(CC(F)F)nc3-c3ccccc3)c2cc1NC(=O)[C@]12COC[C@H]1C2] |
| 277 | 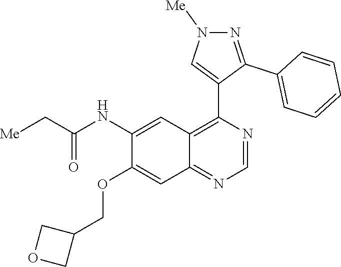 [SMILES: CCC(=O)Nc1cc2c(-c3cn(C)nc3-c3ccccc3)ncnc2cc1OCC1COC1] |
| 278 | 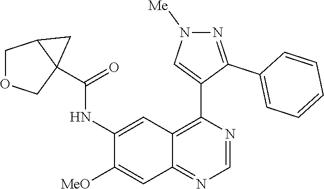 [SMILES: COc1cc2ncnc(-c3cn(C)nc3-c3ccccc3)c2cc1NC(=O)C12COCC1C2] |
| 279 | 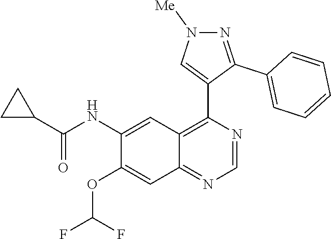 [SMILES: Cn1cc(-c2ncnc3cc(OC(F)F)c(NC(=O)C4CC4)cc23)c(-c2ccccc2)n1] |
| 280 | 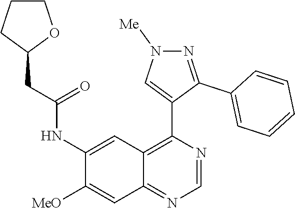 [SMILES: COc1cc2ncnc(-c3cn(C)nc3-c3ccccc3)c2cc1NC(=O)C[C@H]1CCCO1] |
| | or |
| | 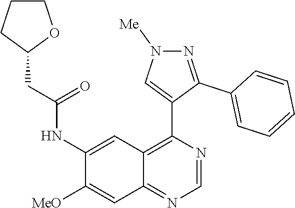 [SMILES: COc1cc2ncnc(-c3cn(C)nc3-c3ccccc3)c2cc1NC(=O)C[C@@H]1CCCO1] |
| 281 | 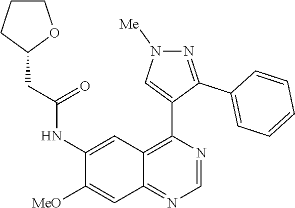 [SMILES: COc1cc2ncnc(-c3cn(C)nc3-c3ccccc3)c2cc1NC(=O)C[C@@H]1CCCO1] |
| | or |
| | 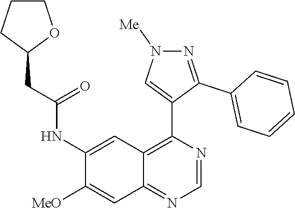 [SMILES: COc1cc2ncnc(-c3cn(C)nc3-c3ccccc3)c2cc1NC(=O)C[C@H]1CCCO1] |
| 282 | 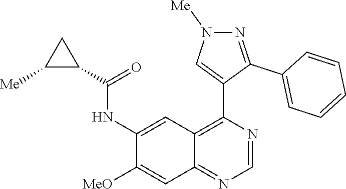 [SMILES: COc1cc2ncnc(-c3cn(C)nc3-c3ccccc3)c2cc1NC(=O)[C@H]1C[C@H]1C] |
| | or |
| | 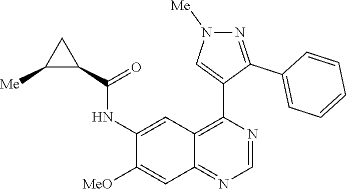 [SMILES: COc1cc2ncnc(-c3cn(C)nc3-c3ccccc3)c2cc1NC(=O)[C@@H]1C[C@@H]1C] |
| 283 | 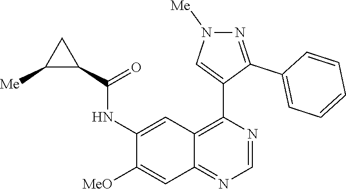 [SMILES: COc1cc2ncnc(-c3cn(C)nc3-c3ccccc3)c2cc1NC(=O)[C@@H]1C[C@@H]1C] |
| | or |
| | 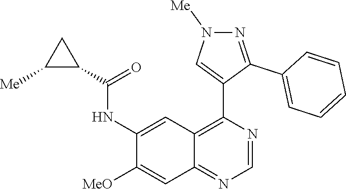 [SMILES: COc1cc2ncnc(-c3cn(C)nc3-c3ccccc3)c2cc1NC(=O)[C@H]1C[C@H]1C] |
| 284 | 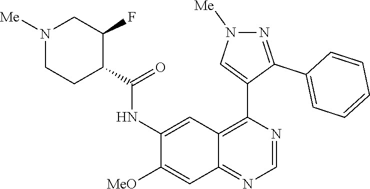 [SMILES: COc1cc2ncnc(-c3cn(C)nc3-c3ccccc3)c2cc1NC(=O)[C@@H]1CCN(C)C[C@H]1F] |
| | or |
| | 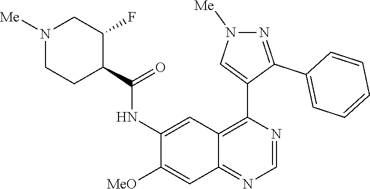 [SMILES: COc1cc2ncnc(-c3cn(C)nc3-c3ccccc3)c2cc1NC(=O)[C@H]1CCN(C)C[C@@H]1F] |
| 285 | 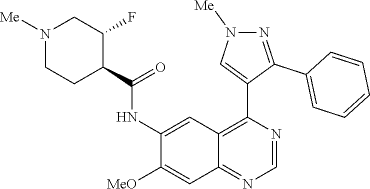 [SMILES: COc1cc2ncnc(-c3cn(C)nc3-c3ccccc3)c2cc1NC(=O)[C@H]1CCN(C)C[C@@H]1F] |
| | or |
| | 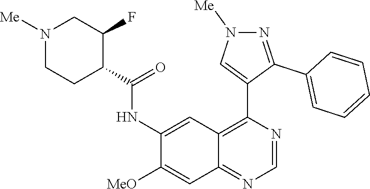 [SMILES: COc1cc2ncnc(-c3cn(C)nc3-c3ccccc3)c2cc1NC(=O)[C@@H]1CCN(C)C[C@H]1F] |
| 286 | 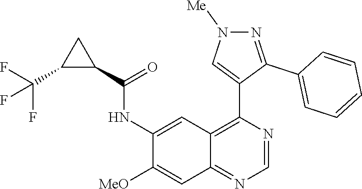 [SMILES: COc1cc2ncnc(-c3cn(C)nc3-c3ccccc3)c2cc1NC(=O)[C@@H]1C[C@H]1C(F)(F)F] |
| | or |
| | 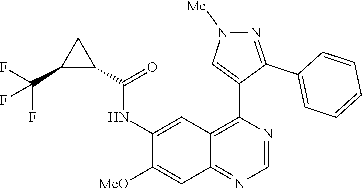 [SMILES: COc1cc2ncnc(-c3cn(C)nc3-c3ccccc3)c2cc1NC(=O)[C@H]1C[C@@H]1C(F)(F)F] |
| 287 | 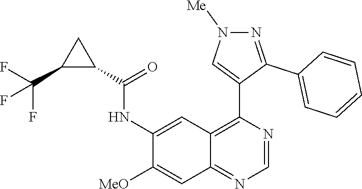 [SMILES: COc1cc2ncnc(-c3cn(C)nc3-c3ccccc3)c2cc1NC(=O)[C@H]1C[C@@H]1C(F)(F)F] |
| | or |
| | 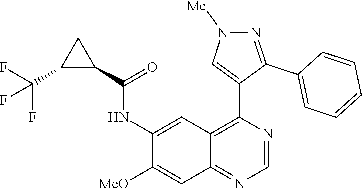 [SMILES: COc1cc2ncnc(-c3cn(C)nc3-c3ccccc3)c2cc1NC(=O)[C@@H]1C[C@H]1C(F)(F)F] |
| 288 | 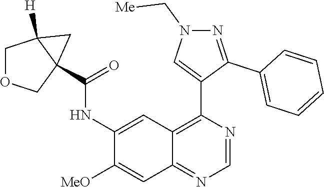 [SMILES: CCn1cc(-c2ncnc3cc(OC)c(NC(=O)[C@@]45COC[C@@H]4C5)cc23)c(-c2ccccc2)n1] |
| | or |
| | 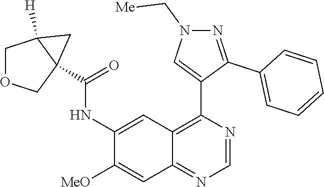 [SMILES: CCn1cc(-c2ncnc3cc(OC)c(NC(=O)[C@]45COC[C@H]4C5)cc23)c(-c2ccccc2)n1] |
| 289 | 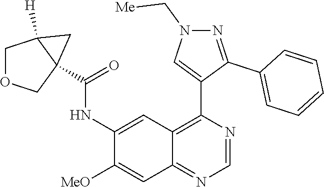 [SMILES: CCn1cc(-c2ncnc3cc(OC)c(NC(=O)[C@]45COC[C@H]4C5)cc23)c(-c2ccccc2)n1] |
| | or |
| | 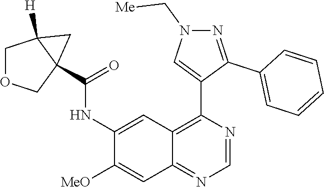 [SMILES: CCn1cc(-c2ncnc3cc(OC)c(NC(=O)[C@@]45COC[C@@H]4C5)cc23)c(-c2ccccc2)n1] |
| 290 | 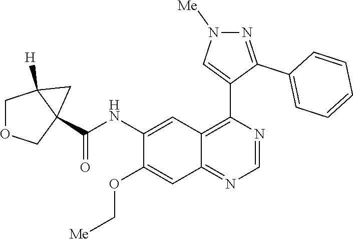 [SMILES: CCOc1cc2ncnc(-c3cn(C)nc3-c3ccccc3)c2cc1NC(=O)[C@@]12COC[C@@H]1C2] |
| | or |
| | 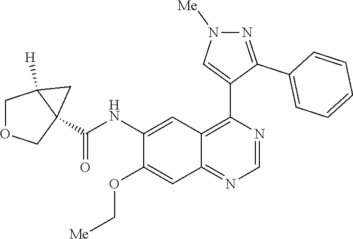 [SMILES: CCOc1cc2ncnc(-c3cn(C)nc3-c3ccccc3)c2cc1NC(=O)[C@]12COC[C@H]1C2] |
| 291 | 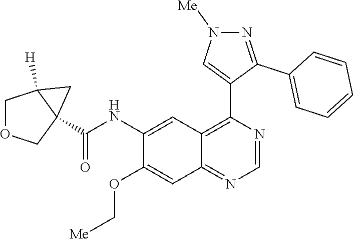 [SMILES: CCOc1cc2ncnc(-c3cn(C)nc3-c3ccccc3)c2cc1NC(=O)[C@]12COC[C@H]1C2] |
| | or |
| | 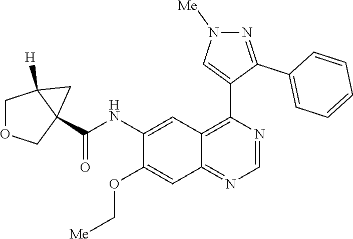 [SMILES: CCOc1cc2ncnc(-c3cn(C)nc3-c3ccccc3)c2cc1NC(=O)[C@@]12COC[C@@H]1C2] |
| 292 | 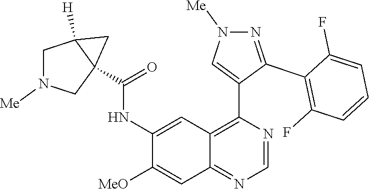 [SMILES: COc1cc2ncnc(-c3cn(C)nc3-c3c(F)cccc3F)c2cc1NC(=O)[C@@]12C[C@@H]1CN(C)C2] |
| | or |
| | 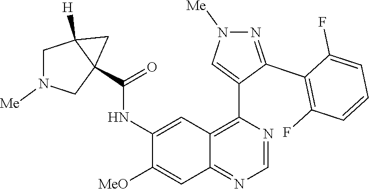 [SMILES: COc1cc2ncnc(-c3cn(C)nc3-c3c(F)cccc3F)c2cc1NC(=O)[C@]12C[C@H]1CN(C)C2] |
| 293 | 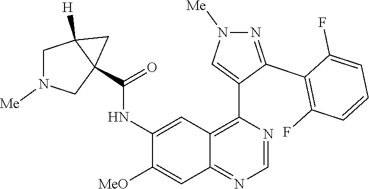 [SMILES: COc1cc2ncnc(-c3cn(C)nc3-c3c(F)cccc3F)c2cc1NC(=O)[C@]12C[C@H]1CN(C)C2] |
| | or |
| | 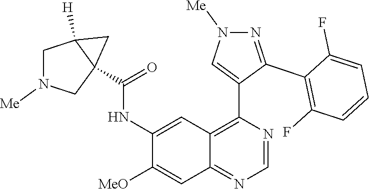 [SMILES: COc1cc2ncnc(-c3cn(C)nc3-c3c(F)cccc3F)c2cc1NC(=O)[C@@]12C[C@@H]1CN(C)C2] |
| 294 | 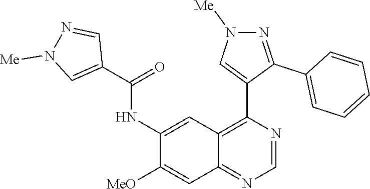 [SMILES: COc1cc2ncnc(-c3cn(C)nc3-c3ccccc3)c2cc1NC(=O)c1cnn(C)c1] |
| 295 | 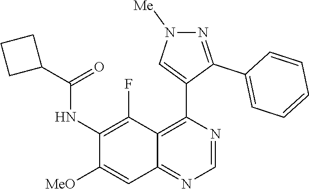 [SMILES: COc1cc2ncnc(-c3cn(C)nc3-c3ccccc3)c2c(F)c1NC(=O)C1CCC1] |
| 296 | 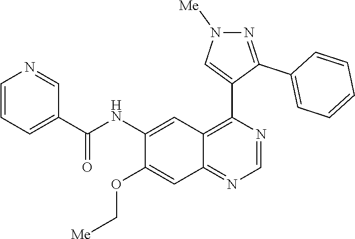 [SMILES: CCOc1cc2ncnc(-c3cn(C)nc3-c3ccccc3)c2cc1NC(=O)c1cccnc1] |
| 297 | 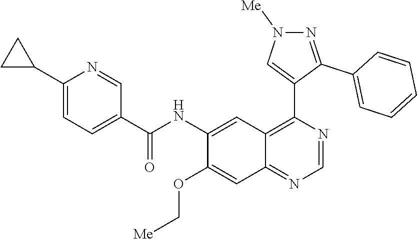 [SMILES: CCOc1cc2ncnc(-c3cn(C)nc3-c3ccccc3)c2cc1NC(=O)c1ccc(C2CC2)nc1] |
| 298 | 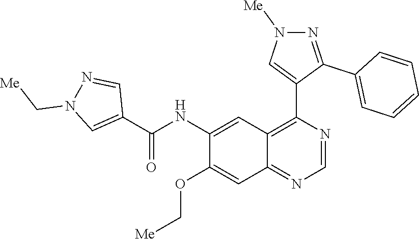 [SMILES: CCOc1cc2ncnc(-c3cn(C)nc3-c3ccccc3)c2cc1NC(=O)c1cnn(CC)c1] |
| 299 | 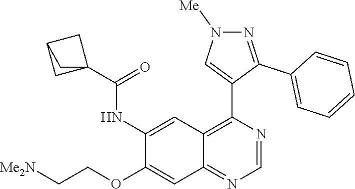 [SMILES: CN(C)CCOc1cc2ncnc(-c3cn(C)nc3-c3ccccc3)c2cc1NC(=O)C12CC(C1)C2] |
| 300 | 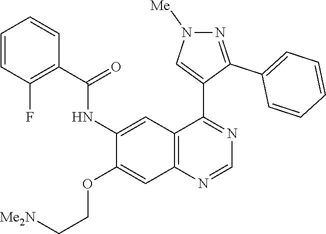 [SMILES: CN(C)CCOc1cc2ncnc(-c3cn(C)nc3-c3ccccc3)c2cc1NC(=O)c1ccccc1F] |
| 301 | 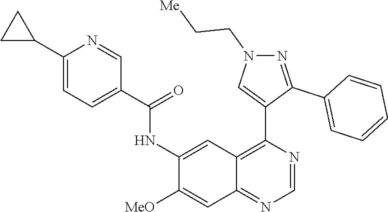 [SMILES: CCCn1cc(-c2ncnc3cc(OC)c(NC(=O)c4ccc(C5CC5)nc4)cc23)c(-c2ccccc2)n1] |
| 302 | 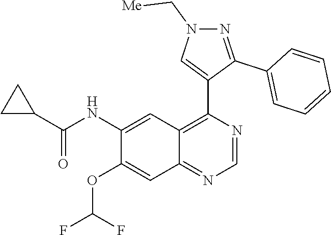 [SMILES: CCn1cc(-c2ncnc3cc(OC(F)F)c(NC(=O)C4CC4)cc23)c(-c2ccccc2)n1] |
| 303 | 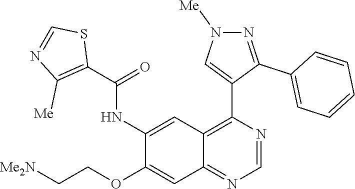 [SMILES: Cc1ncsc1C(=O)Nc1cc2c(-c3cn(C)nc3-c3ccccc3)ncnc2cc1OCCN(C)C] |
| 304 | 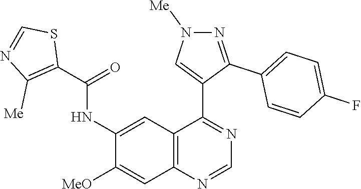 [SMILES: COc1cc2ncnc(-c3cn(C)nc3-c3ccc(F)cc3)c2cc1NC(=O)c1scnc1C] |
| 305 | 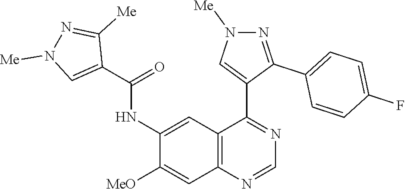 [SMILES: COc1cc2ncnc(-c3cn(C)nc3-c3ccc(F)cc3)c2cc1NC(=O)c1cn(C)nc1C] |
| 306 | 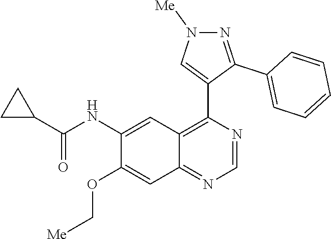 [SMILES: CCOc1cc2ncnc(-c3cn(C)nc3-c3ccccc3)c2cc1NC(=O)C1CC1] |
| 307 | 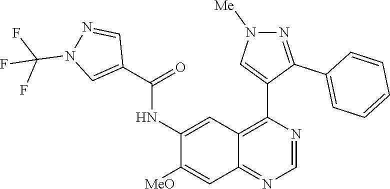 [SMILES: COc1cc2ncnc(-c3cn(C)nc3-c3ccccc3)c2cc1NC(=O)c1cnn(C(F)(F)F)c1] |
| 308 | 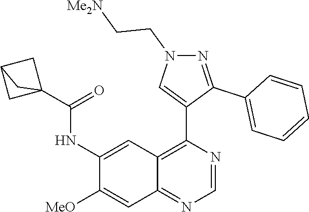 [SMILES: COc1cc2ncnc(-c3cn(CCN(C)C)nc3-c3ccccc3)c2cc1NC(=O)C12CC(C1)C2] |
| 309 | 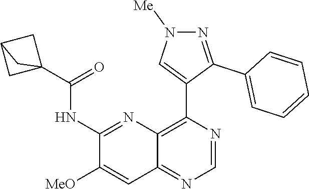 [SMILES: COc1cc2ncnc(-c3cn(C)nc3-c3ccccc3)c2nc1NC(=O)C12CC(C1)C2] |
| 310 | 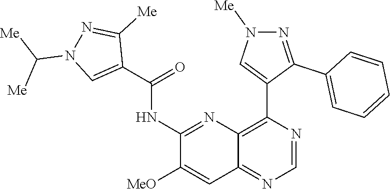 [SMILES: COc1cc2ncnc(-c3cn(C)nc3-c3ccccc3)c2nc1NC(=O)c1cn(C(C)C)nc1C] |
| 311 | 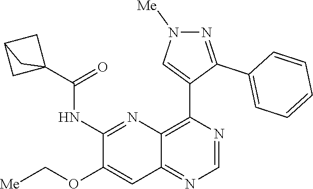 [SMILES: CCOc1cc2ncnc(-c3cn(C)nc3-c3ccccc3)c2nc1NC(=O)C12CC(C1)C2] |
| 312 | 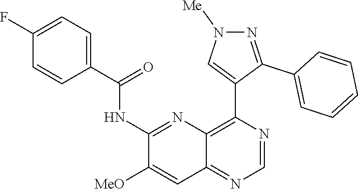 [SMILES: COc1cc2ncnc(-c3cn(C)nc3-c3ccccc3)c2nc1NC(=O)c1ccc(F)cc1] |
| 313 | 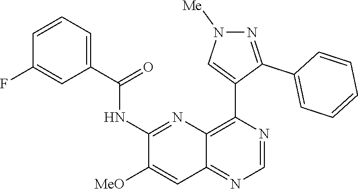 [SMILES: COc1cc2ncnc(-c3cn(C)nc3-c3ccccc3)c2nc1NC(=O)c1cccc(F)c1] |
| 314 | 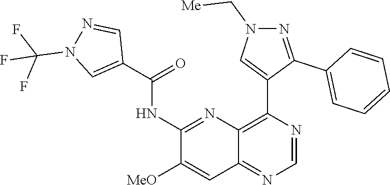 [SMILES: CCn1cc(-c2ncnc3cc(OC)c(NC(=O)c4cnn(C(F)(F)F)c4)nc23)c(-c2ccccc2)n1] |
| 315 | 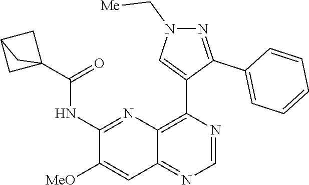 [SMILES: CCn1cc(-c2ncnc3cc(OC)c(NC(=O)C45CC(C4)C5)nc23)c(-c2ccccc2)n1] |
| 316 | 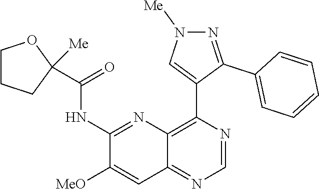 [SMILES: COc1cc2ncnc(-c3cn(C)nc3-c3ccccc3)c2nc1NC(=O)C1(C)CCCO1] |
| 317 | 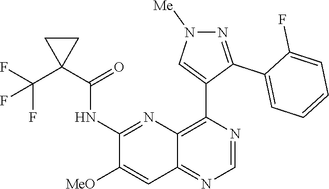 [SMILES: COc1cc2ncnc(-c3cn(C)nc3-c3ccccc3F)c2nc1NC(=O)C1(C(F)(F)F)CC1] |
| 318 | 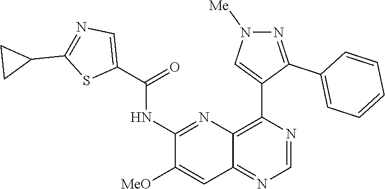 [SMILES: COc1cc2ncnc(-c3cn(C)nc3-c3ccccc3)c2nc1NC(=O)c1cnc(C2CC2)s1] |
| 319 | 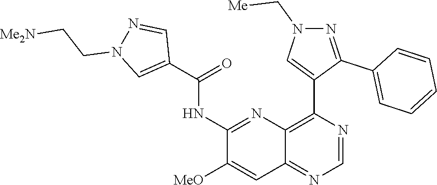 [SMILES: CCn1cc(-c2ncnc3cc(OC)c(NC(=O)c4cnn(CCN(C)C)c4)nc23)c(-c2ccccc2)n1] |
| 320 | 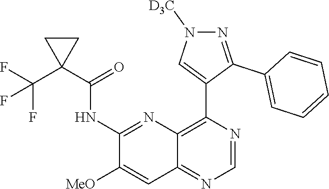 [SMILES: [2H]C([2H])([2H])n1cc(-c2ncnc3cc(OC)c(NC(=O)C4(C(F)(F)F)CC4)nc23)c(-c2ccccc2)n1] |
| 321 | 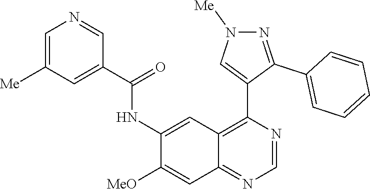 [SMILES: COc1cc2ncnc(-c3cn(C)nc3-c3ccccc3)c2cc1NC(=O)c1cncc(C)c1] |
| 322 | 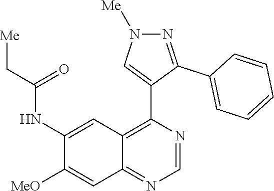 [SMILES: CCC(=O)Nc1cc2c(-c3cn(C)nc3-c3ccccc3)ncnc2cc1OC] |
| 323 | 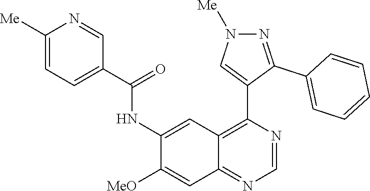 [SMILES: COc1cc2ncnc(-c3cn(C)nc3-c3ccccc3)c2cc1NC(=O)c1ccc(C)nc1] |
| 324 | 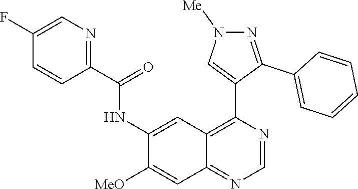 [SMILES: COc1cc2ncnc(-c3cn(C)nc3-c3ccccc3)c2cc1NC(=O)c1ccc(F)cn1] |
| 325 | 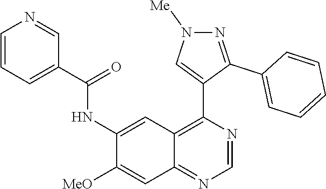 [SMILES: COc1cc2ncnc(-c3cn(C)nc3-c3ccccc3)c2cc1NC(=O)c1cccnc1] |
| 326 | 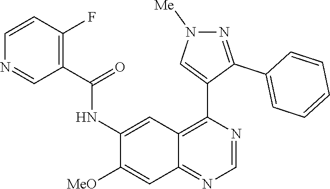 [SMILES: COc1cc2ncnc(-c3cn(C)nc3-c3ccccc3)c2cc1NC(=O)c1cnccc1F] |
| 327 | 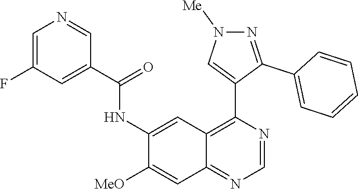 [SMILES: COc1cc2ncnc(-c3cn(C)nc3-c3ccccc3)c2cc1NC(=O)c1cncc(F)c1] |
| 328 | 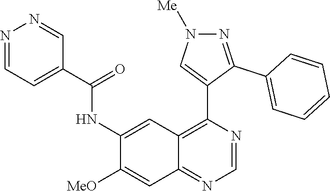 [SMILES: COc1cc2ncnc(-c3cn(C)nc3-c3ccccc3)c2cc1NC(=O)c1ccnnc1] |
| 329 | 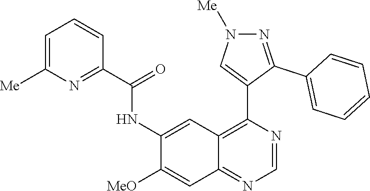 [SMILES: COc1cc2ncnc(-c3cn(C)nc3-c3ccccc3)c2cc1NC(=O)c1cccc(C)n1] |
| 330 | 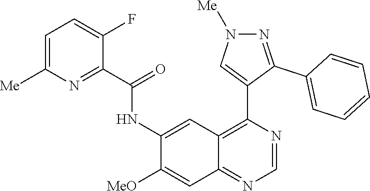 [SMILES: COc1cc2ncnc(-c3cn(C)nc3-c3ccccc3)c2cc1NC(=O)c1nc(C)ccc1F] |
| 331 | 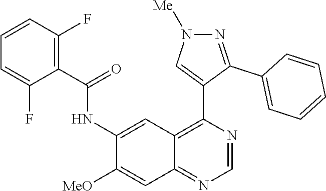 [SMILES: COc1cc2ncnc(-c3cn(C)nc3-c3ccccc3)c2cc1NC(=O)c1c(F)cccc1F] |
| 332 | 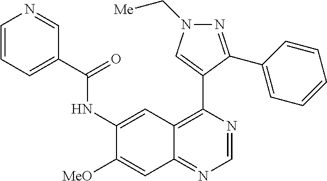 [SMILES: CCn1cc(-c2ncnc3cc(OC)c(NC(=O)c4cccnc4)cc23)c(-c2ccccc2)n1] |
| 333 | 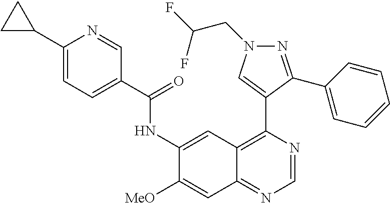 [SMILES: COc1cc2ncnc(-c3cn(CC(F)F)nc3-c3ccccc3)c2cc1NC(=O)c1ccc(C2CC2)nc1] |
| 334 | 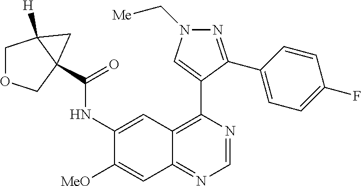 [SMILES: CCn1cc(-c2ncnc3cc(OC)c(NC(=O)[C@@]45COC[C@@H]4C5)cc23)c(-c2ccc(F)cc2)n1] |
| | or |
| | 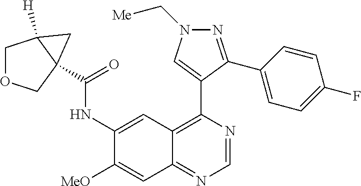 [SMILES: CCn1cc(-c2ncnc3cc(OC)c(NC(=O)[C@]45COC[C@H]4C5)cc23)c(-c2ccc(F)cc2)n1] |
| 335 | 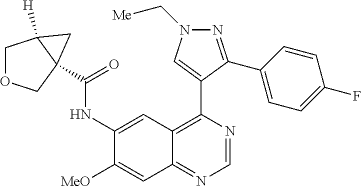 [SMILES: CCn1cc(-c2ncnc3cc(OC)c(NC(=O)[C@]45COC[C@H]4C5)cc23)c(-c2ccc(F)cc2)n1] |
| | or |
| | 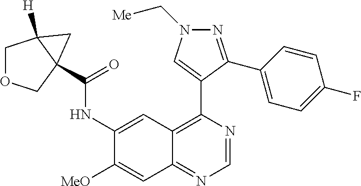 [SMILES: CCn1cc(-c2ncnc3cc(OC)c(NC(=O)[C@@]45COC[C@@H]4C5)cc23)c(-c2ccc(F)cc2)n1] |
| 336 | 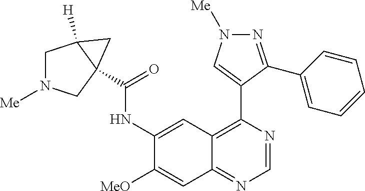 [SMILES: COc1cc2ncnc(-c3cn(C)nc3-c3ccccc3)c2cc1NC(=O)[C@@]12C[C@@H]1CN(C)C2] |
| | or |
| | 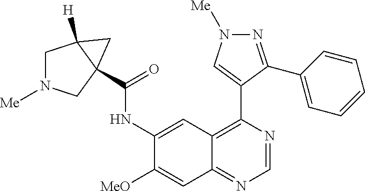 [SMILES: COc1cc2ncnc(-c3cn(C)nc3-c3ccccc3)c2cc1NC(=O)[C@]12C[C@H]1CN(C)C2] |
| 337 | 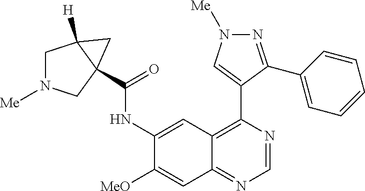 [SMILES: COc1cc2ncnc(-c3cn(C)nc3-c3ccccc3)c2cc1NC(=O)[C@]12C[C@H]1CN(C)C2] |
| | or |
| | 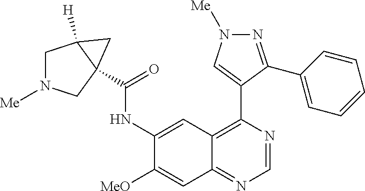 [SMILES: COc1cc2ncnc(-c3cn(C)nc3-c3ccccc3)c2cc1NC(=O)[C@@]12C[C@@H]1CN(C)C2] |
| 338 | 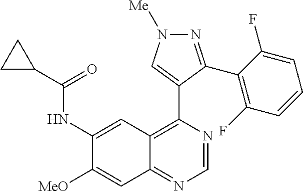 [SMILES: COc1cc2ncnc(-c3cn(C)nc3-c3c(F)cccc3F)c2cc1NC(=O)C1CC1] |
| 339 | 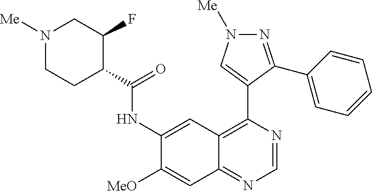 [SMILES: COc1cc2ncnc(-c3cn(C)nc3-c3ccccc3)c2cc1NC(=O)[C@@H]1CCN(C)C[C@H]1F] |
| 340 | 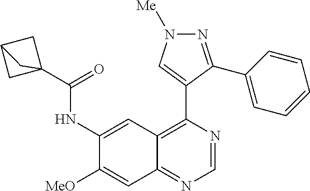 [SMILES: COc1cc2ncnc(-c3cn(C)nc3-c3ccccc3)c2cc1NC(=O)C12CC(C1)C2] |
| 341 | 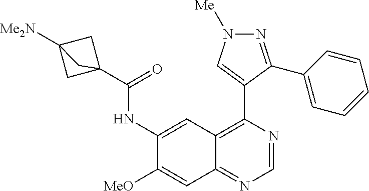 [SMILES: COc1cc2ncnc(-c3cn(C)nc3-c3ccccc3)c2cc1NC(=O)C12CC(N(C)C)(C1)C2] |
| 342 | 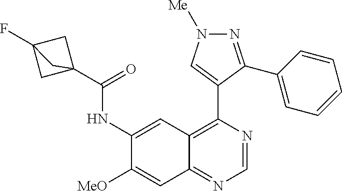 [SMILES: COc1cc2ncnc(-c3cn(C)nc3-c3ccccc3)c2cc1NC(=O)C12CC(F)(C1)C2] |
| 343 | 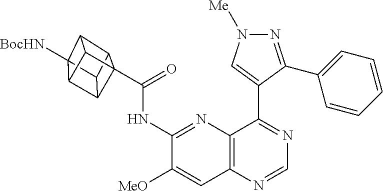 [SMILES: COc1cc2ncnc(-c3cn(C)nc3-c3ccccc3)c2nc1NC(=O)C12C3C4C1C1C2C3C41NC(=O)OC(C)(C)C] |
| 344 | 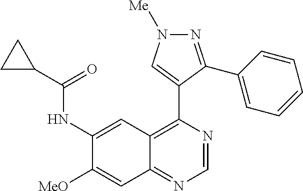 [SMILES: COc1cc2ncnc(-c3cn(C)nc3-c3ccccc3)c2cc1NC(=O)C1CC1] |
| 345 | 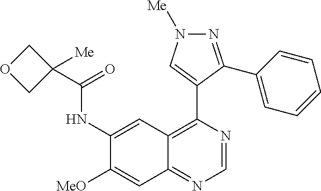 [SMILES: COc1cc2ncnc(-c3cn(C)nc3-c3ccccc3)c2cc1NC(=O)C1(C)COC1] |
| 346 | 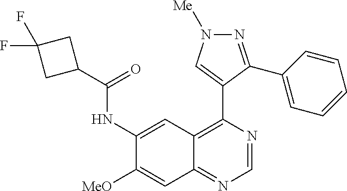 [SMILES: COc1cc2ncnc(-c3cn(C)nc3-c3ccccc3)c2cc1NC(=O)C1CC(F)(F)C1] |
| 347 | 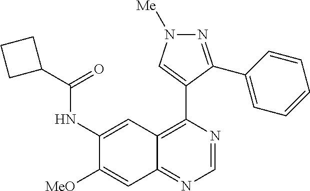 [SMILES: COc1cc2ncnc(-c3cn(C)nc3-c3ccccc3)c2cc1NC(=O)C1CCC1] |
| 348 | 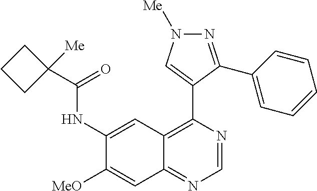 [SMILES: COc1cc2ncnc(-c3cn(C)nc3-c3ccccc3)c2cc1NC(=O)C1(C)CCC1] |
| 349 | 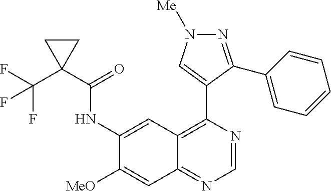 [SMILES: COc1cc2ncnc(-c3cn(C)nc3-c3ccccc3)c2cc1NC(=O)C1(C(F)(F)F)CC1] |
| 350 | 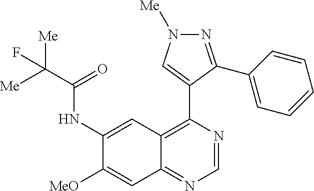 [SMILES: COc1cc2ncnc(-c3cn(C)nc3-c3ccccc3)c2cc1NC(=O)C(C)(C)F] |
| 351 | 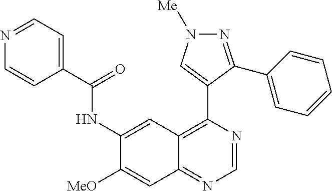 [SMILES: COc1cc2ncnc(-c3cn(C)nc3-c3ccccc3)c2cc1NC(=O)c1ccncc1] |
| 352 | 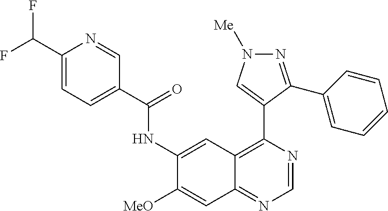 [SMILES: COc1cc2ncnc(-c3cn(C)nc3-c3ccccc3)c2cc1NC(=O)c1ccc(C(F)F)nc1] |
| 353 | 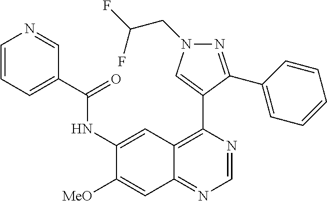 [SMILES: COc1cc2ncnc(-c3cn(CC(F)F)nc3-c3ccccc3)c2cc1NC(=O)c1cccnc1] |
| 354 | 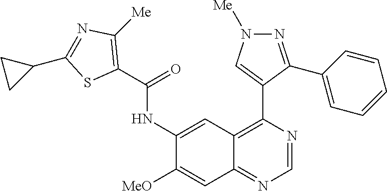 [SMILES: COc1cc2ncnc(-c3cn(C)nc3-c3ccccc3)c2cc1NC(=O)c1sc(C2CC2)nc1C] |
| 355 | 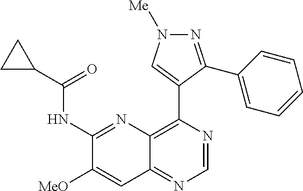 [SMILES: COc1cc2ncnc(-c3cn(C)nc3-c3ccccc3)c2nc1NC(=O)C1CC1] |
| 356 | 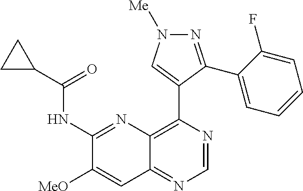 [SMILES: COc1cc2ncnc(-c3cn(C)nc3-c3ccccc3F)c2nc1NC(=O)C1CC1] |
| 357 | 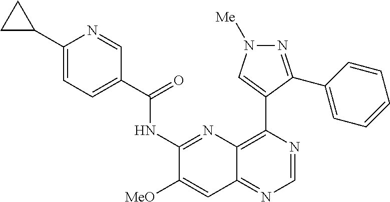 [SMILES: COc1cc2ncnc(-c3cn(C)nc3-c3ccccc3)c2nc1NC(=O)c1ccc(C2CC2)nc1] |
| 358 | 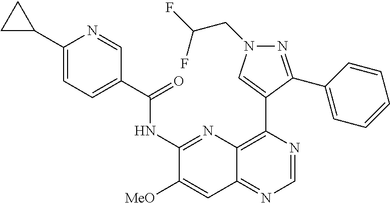 [SMILES: COc1cc2ncnc(-c3cn(CC(F)F)nc3-c3ccccc3)c2nc1NC(=O)c1ccc(C2CC2)nc1] |
| 359 | 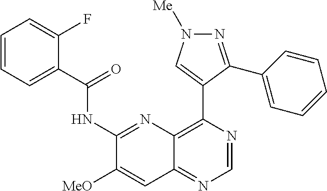 [SMILES: COc1cc2ncnc(-c3cn(C)nc3-c3ccccc3)c2nc1NC(=O)c1ccccc1F] |
| 360 | 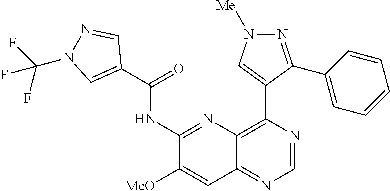 [SMILES: COc1cc2ncnc(-c3cn(C)nc3-c3ccccc3)c2nc1NC(=O)c1cnn(C(F)(F)F)c1] |
| 361 | 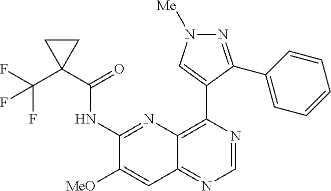 [SMILES: COc1cc2ncnc(-c3cn(C)nc3-c3ccccc3)c2nc1NC(=O)C1(C(F)(F)F)CC1] |
| 362 | 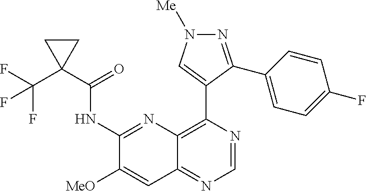 [SMILES: COc1cc2ncnc(-c3cn(C)nc3-c3ccc(F)cc3)c2nc1NC(=O)C1(C(F)(F)F)CC1] |
| 363 | 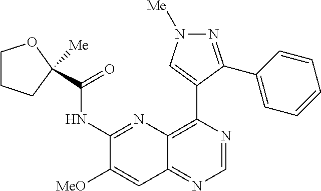 [SMILES: COc1cc2ncnc(-c3cn(C)nc3-c3ccccc3)c2nc1NC(=O)[C@@]1(C)CCCO1] |
| 364 | 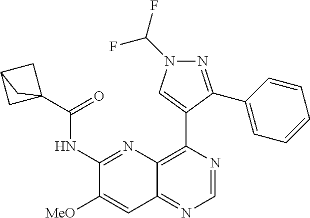 [SMILES: COc1cc2ncnc(-c3cn(C(F)F)nc3-c3ccccc3)c2nc1NC(=O)C12CC(C1)C2] |
| 365 | 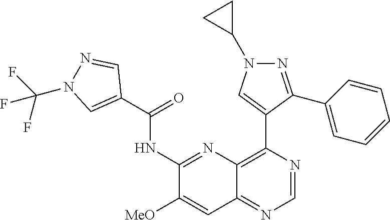 [SMILES: COc1cc2ncnc(-c3cn(C4CC4)nc3-c3ccccc3)c2nc1NC(=O)c1cnn(C(F)(F)F)c1] |
| 366 | 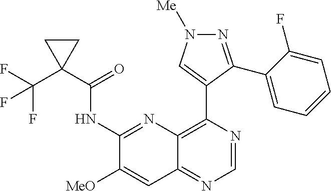 [SMILES: COc1cc2ncnc(-c3cn(C)nc3-c3ccccc3F)c2nc1NC(=O)C1(C(F)(F)F)CC1] |
| 367 | 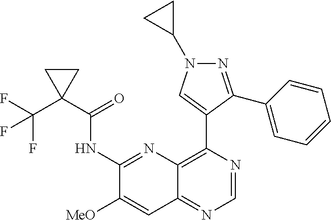 [SMILES: COc1cc2ncnc(-c3cn(C4CC4)nc3-c3ccccc3)c2nc1NC(=O)C1(C(F)(F)F)CC1] |
| 368 | 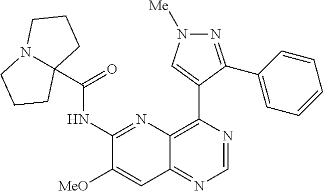 [SMILES: COc1cc2ncnc(-c3cn(C)nc3-c3ccccc3)c2nc1NC(=O)C12CCCN1CCC2] |
| 369 | 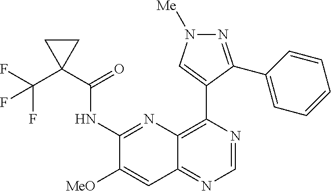 [SMILES: COc1cc2ncnc(-c3cn(C)nc3-c3ccccc3)c2nc1NC(=O)C1(C(F)(F)F)CC1] |
| 370 | 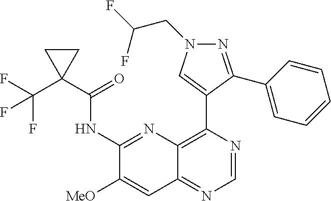 [SMILES: COc1cc2ncnc(-c3cn(CC(F)F)nc3-c3ccccc3)c2nc1NC(=O)C1(C(F)(F)F)CC1] |
| 371 | 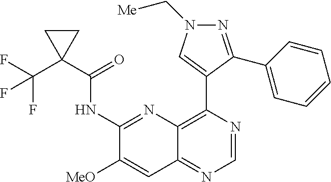 [SMILES: CCn1cc(-c2ncnc3cc(OC)c(NC(=O)C4(C(F)(F)F)CC4)nc23)c(-c2ccccc2)n1] |
| 372 | 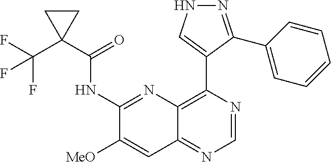 [SMILES: COc1cc2ncnc(-c3c[nH]nc3-c3ccccc3)c2nc1NC(=O)C1(C(F)(F)F)CC1] |
| 373 | 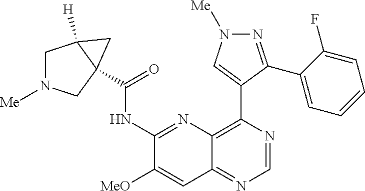 [SMILES: COc1cc2ncnc(-c3cn(C)nc3-c3ccccc3F)c2nc1NC(=O)[C@@]12C[C@@H]1CN(C)C2] |
| | or |
| | 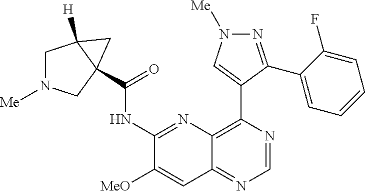 [SMILES: COc1cc2ncnc(-c3cn(C)nc3-c3ccccc3F)c2nc1NC(=O)[C@]12C[C@H]1CN(C)C2] |
| 374 | 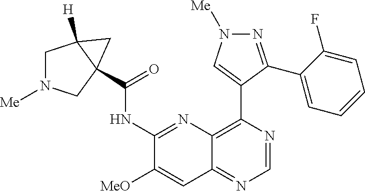 [SMILES: COc1cc2ncnc(-c3cn(C)nc3-c3ccccc3F)c2nc1NC(=O)[C@]12C[C@H]1CN(C)C2] |
| | or |
| | 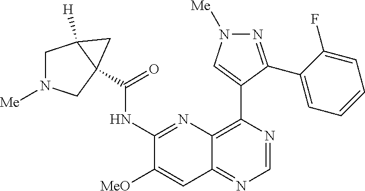 [SMILES: COc1cc2ncnc(-c3cn(C)nc3-c3ccccc3F)c2nc1NC(=O)[C@@]12C[C@@H]1CN(C)C2] |
| 375 | 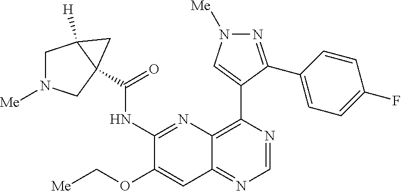 [SMILES: CCOc1cc2ncnc(-c3cn(C)nc3-c3ccc(F)cc3)c2nc1NC(=O)[C@@]12C[C@@H]1CN(C)C2] |
| | or |
| | 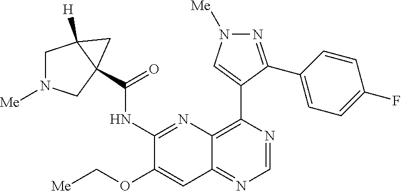 [SMILES: CCOc1cc2ncnc(-c3cn(C)nc3-c3ccc(F)cc3)c2nc1NC(=O)[C@]12C[C@H]1CN(C)C2] |
| 376 | 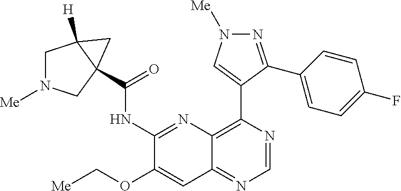 [SMILES: CCOc1cc2ncnc(-c3cn(C)nc3-c3ccc(F)cc3)c2nc1NC(=O)[C@]12C[C@H]1CN(C)C2] |
| | or |
| | 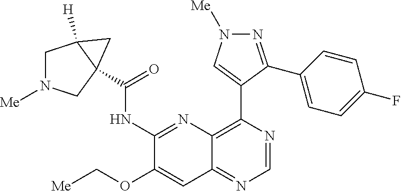 [SMILES: CCOc1cc2ncnc(-c3cn(C)nc3-c3ccc(F)cc3)c2nc1NC(=O)[C@@]12C[C@@H]1CN(C)C2] |
| 377 | 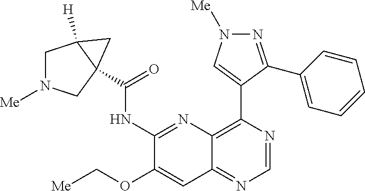 [SMILES: CCOc1cc2ncnc(-c3cn(C)nc3-c3ccccc3)c2nc1NC(=O)[C@@]12C[C@@H]1CN(C)C2] |
| | or |
| | 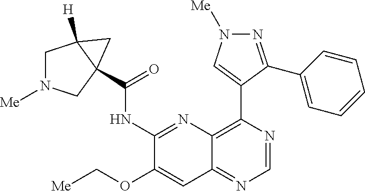 [SMILES: CCOc1cc2ncnc(-c3cn(C)nc3-c3ccccc3)c2nc1NC(=O)[C@]12C[C@H]1CN(C)C2] |
| 378 | 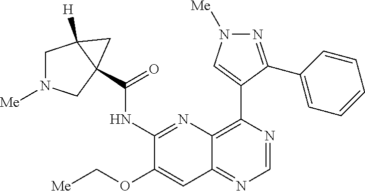 [SMILES: CCOc1cc2ncnc(-c3cn(C)nc3-c3ccccc3)c2nc1NC(=O)[C@]12C[C@H]1CN(C)C2] |
| | or |
| | 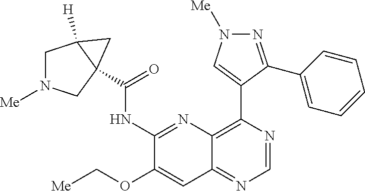 [SMILES: CCOc1cc2ncnc(-c3cn(C)nc3-c3ccccc3)c2nc1NC(=O)[C@@]12C[C@@H]1CN(C)C2] |
| 379 | 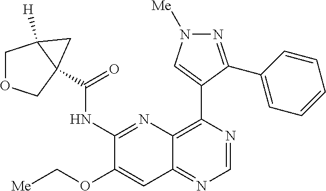 [SMILES: CCOc1cc2ncnc(-c3cn(C)nc3-c3ccccc3)c2nc1NC(=O)[C@]12COC[C@H]1C2] |
| | or |
| | 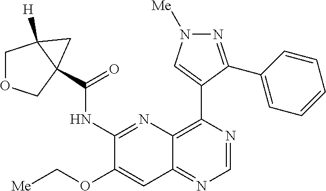 [SMILES: CCOc1cc2ncnc(-c3cn(C)nc3-c3ccccc3)c2nc1NC(=O)[C@@]12COC[C@@H]1C2] |
| 380 | 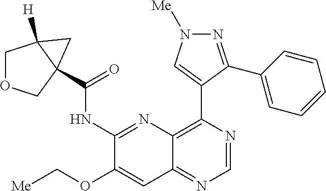 [SMILES: CCOc1cc2ncnc(-c3cn(C)nc3-c3ccccc3)c2nc1NC(=O)[C@@]12COC[C@@H]1C2] |
| | or |
| | 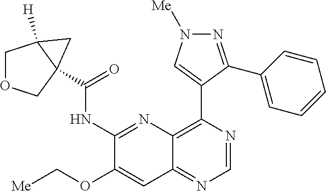 [SMILES: CCOc1cc2ncnc(-c3cn(C)nc3-c3ccccc3)c2nc1NC(=O)[C@]12COC[C@H]1C2] |
| 381 | 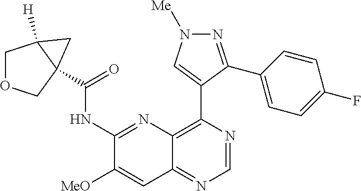 [SMILES: COc1cc2ncnc(-c3cn(C)nc3-c3ccc(F)cc3)c2nc1NC(=O)[C@]12COC[C@H]1C2] |
| | or |
| | 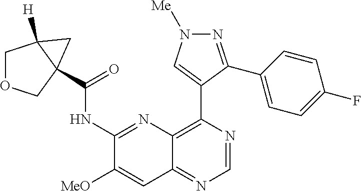 [SMILES: COc1cc2ncnc(-c3cn(C)nc3-c3ccc(F)cc3)c2nc1NC(=O)[C@@]12COC[C@@H]1C2] |
| 382 | 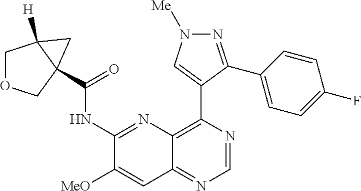 [SMILES: COc1cc2ncnc(-c3cn(C)nc3-c3ccc(F)cc3)c2nc1NC(=O)[C@@]12COC[C@@H]1C2] |
| | or |
| | 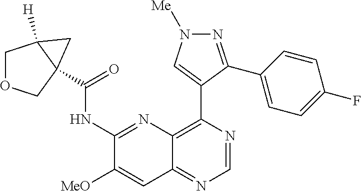 [SMILES: COc1cc2ncnc(-c3cn(C)nc3-c3ccc(F)cc3)c2nc1NC(=O)[C@]12COC[C@H]1C2] |
| 383 | 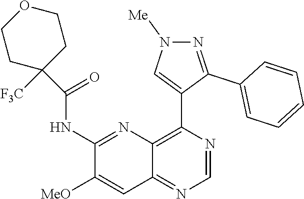 [SMILES: COc1cc2ncnc(-c3cn(C)nc3-c3ccccc3)c2nc1NC(=O)C1(C(F)(F)F)CCOCC1] |
| 384 | 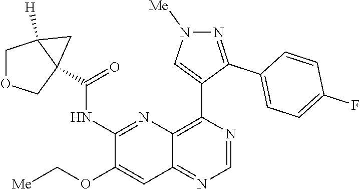 [SMILES: CCOc1cc2ncnc(-c3cn(C)nc3-c3ccc(F)cc3)c2nc1NC(=O)[C@]12COC[C@H]1C2] |
| | or |
| | 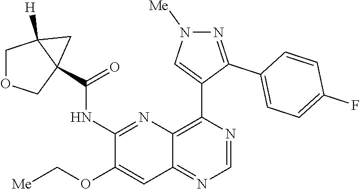 [SMILES: CCOc1cc2ncnc(-c3cn(C)nc3-c3ccc(F)cc3)c2nc1NC(=O)[C@@]12COC[C@@H]1C2] |
| 385 | 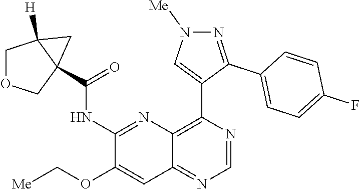 [SMILES: CCOc1cc2ncnc(-c3cn(C)nc3-c3ccc(F)cc3)c2nc1NC(=O)[C@@]12COC[C@@H]1C2] |
| | or |
| | 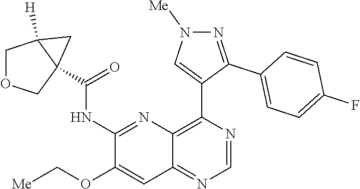 [SMILES: CCOc1cc2ncnc(-c3cn(C)nc3-c3ccc(F)cc3)c2nc1NC(=O)[C@]12COC[C@H]1C2] |
| 386 | 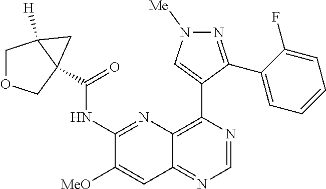 [SMILES: COc1cc2ncnc(-c3cn(C)nc3-c3ccccc3F)c2nc1NC(=O)[C@]12COC[C@H]1C2] |
| | or |
| | 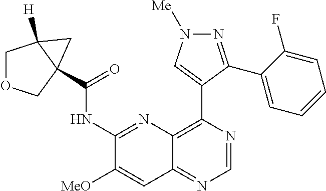 [SMILES: COc1cc2ncnc(-c3cn(C)nc3-c3ccccc3F)c2nc1NC(=O)[C@@]12COC[C@@H]1C2] |
| 387 | 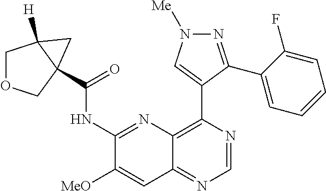 [SMILES: COc1cc2ncnc(-c3cn(C)nc3-c3ccccc3F)c2nc1NC(=O)[C@@]12COC[C@@H]1C2] |
| | or |
| | 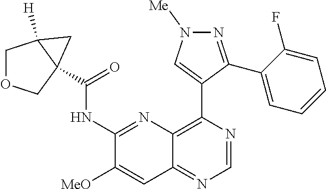 [SMILES: COc1cc2ncnc(-c3cn(C)nc3-c3ccccc3F)c2nc1NC(=O)[C@]12COC[C@H]1C2] |
| 388 | 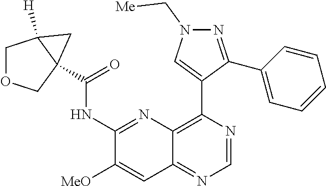 [SMILES: CCn1cc(-c2ncnc3cc(OC)c(NC(=O)[C@]45COC[C@H]4C5)nc23)c(-c2ccccc2)n1] |
| | or |
| | 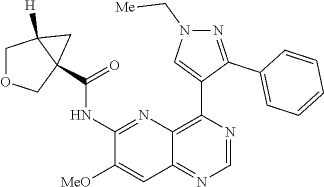 [SMILES: CCn1cc(-c2ncnc3cc(OC)c(NC(=O)[C@@]45COC[C@@H]4C5)nc23)c(-c2ccccc2)n1] |
| 389 | 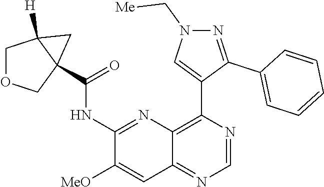 [SMILES: CCn1cc(-c2ncnc3cc(OC)c(NC(=O)[C@@]45COC[C@@H]4C5)nc23)c(-c2ccccc2)n1] |
| | or |
| | 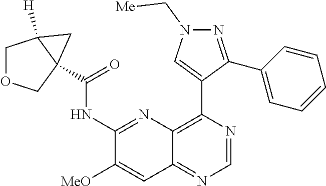 [SMILES: CCn1cc(-c2ncnc3cc(OC)c(NC(=O)[C@]45COC[C@H]4C5)nc23)c(-c2ccccc2)n1] |
| 390 | 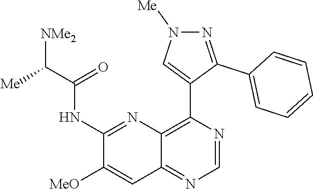 [SMILES: COc1cc2ncnc(-c3cn(C)nc3-c3ccccc3)c2nc1NC(=O)[C@H](C)N(C)C] |
| 391 | 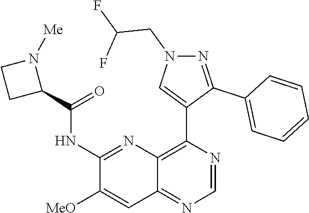 [SMILES: COc1cc2ncnc(-c3cn(CC(F)F)nc3-c3ccccc3)c2nc1NC(=O)[C@H]1CCN1C] |
| 392 | 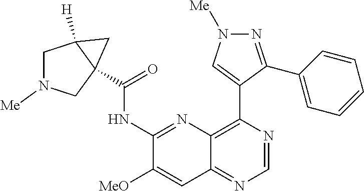 [SMILES: COc1cc2ncnc(-c3cn(C)nc3-c3ccccc3)c2nc1NC(=O)[C@@]12C[C@@H]1CN(C)C2] |
| | or |
| | 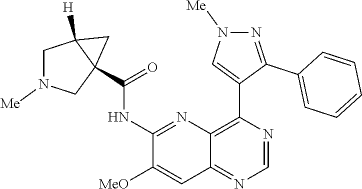 [SMILES: COc1cc2ncnc(-c3cn(C)nc3-c3ccccc3)c2nc1NC(=O)[C@]12C[C@H]1CN(C)C2] |
| 393 | 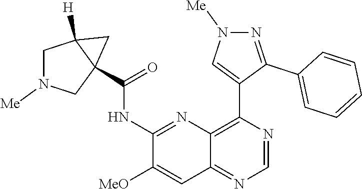 [SMILES: COc1cc2ncnc(-c3cn(C)nc3-c3ccccc3)c2nc1NC(=O)[C@]12C[C@H]1CN(C)C2] |
| | or |
| | 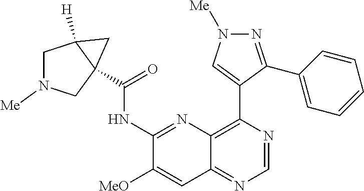 [SMILES: COc1cc2ncnc(-c3cn(C)nc3-c3ccccc3)c2nc1NC(=O)[C@@]12C[C@@H]1CN(C)C2] |
| 394 | 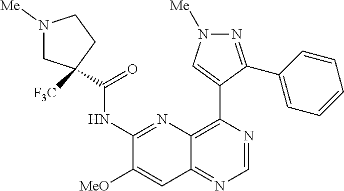 [SMILES: COc1cc2ncnc(-c3cn(C)nc3-c3ccccc3)c2nc1NC(=O)[C@@]1(C(F)(F)F)CCN(C)C1] |
| | or |
| | 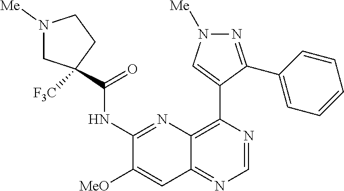 [SMILES: COc1cc2ncnc(-c3cn(C)nc3-c3ccccc3)c2nc1NC(=O)[C@]1(C(F)(F)F)CCN(C)C1] |
| 395 | 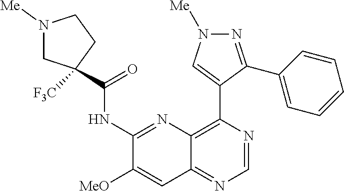 [SMILES: COc1cc2ncnc(-c3cn(C)nc3-c3ccccc3)c2nc1NC(=O)[C@]1(C(F)(F)F)CCN(C)C1] |
| | or |
| | 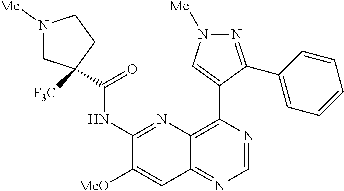 [SMILES: COc1cc2ncnc(-c3cn(C)nc3-c3ccccc3)c2nc1NC(=O)[C@@]1(C(F)(F)F)CCN(C)C1] |
| 396 | 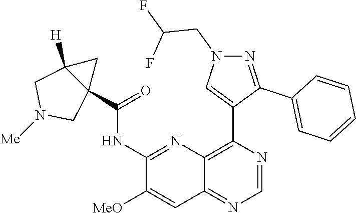 [SMILES: COc1cc2ncnc(-c3cn(CC(F)F)nc3-c3ccccc3)c2nc1NC(=O)[C@]12C[C@H]1CN(C)C2] |
| | or |
| | 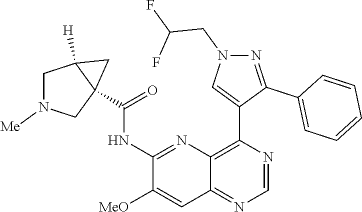 [SMILES: COc1cc2ncnc(-c3cn(CC(F)F)nc3-c3ccccc3)c2nc1NC(=O)[C@@]12C[C@@H]1CN(C)C2] |
| 397 | 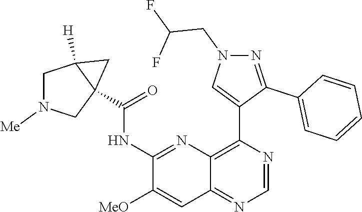 [SMILES: COc1cc2ncnc(-c3cn(CC(F)F)nc3-c3ccccc3)c2nc1NC(=O)[C@@]12C[C@@H]1CN(C)C2] |
| | or |
| | 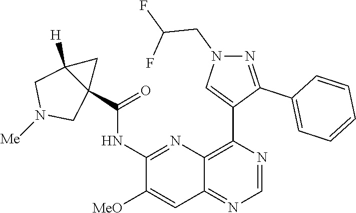 [SMILES: COc1cc2ncnc(-c3cn(CC(F)F)nc3-c3ccccc3)c2nc1NC(=O)[C@]12C[C@H]1CN(C)C2] |
| 398 | 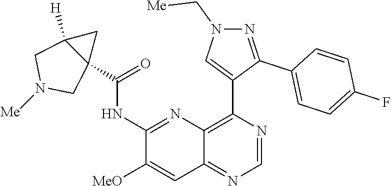 [SMILES: CCn1cc(-c2ncnc3cc(OC)c(NC(=O)[C@@]45C[C@@H]4CN(C)C5)nc23)c(-c2ccc(F)cc2)n1] |
| | or |
| | 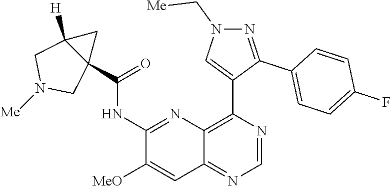 [SMILES: CCn1cc(-c2ncnc3cc(OC)c(NC(=O)[C@]45C[C@H]4CN(C)C5)nc23)c(-c2ccc(F)cc2)n1] |
| 399 | 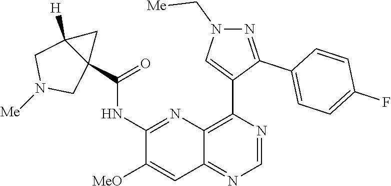 [SMILES: CCn1cc(-c2ncnc3cc(OC)c(NC(=O)[C@]45C[C@H]4CN(C)C5)nc23)c(-c2ccc(F)cc2)n1] |
| | or |
| | 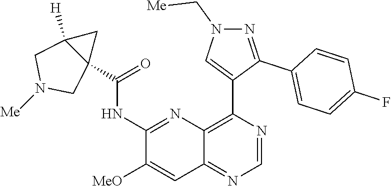 [SMILES: CCn1cc(-c2ncnc3cc(OC)c(NC(=O)[C@@]45C[C@@H]4CN(C)C5)nc23)c(-c2ccc(F)cc2)n1] |
| 400 | 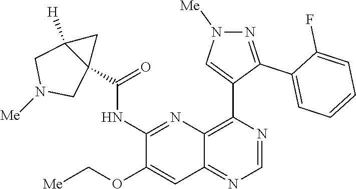 [SMILES: CCOc1cc2ncnc(-c3cn(C)nc3-c3ccccc3F)c2nc1NC(=O)[C@@]12C[C@@H]1CN(C)C2] |
| | or |
| | 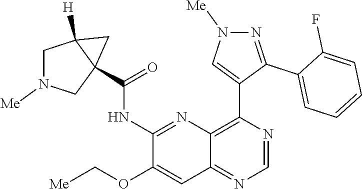 [SMILES: CCOc1cc2ncnc(-c3cn(C)nc3-c3ccccc3F)c2nc1NC(=O)[C@]12C[C@H]1CN(C)C2] |
| 401 | 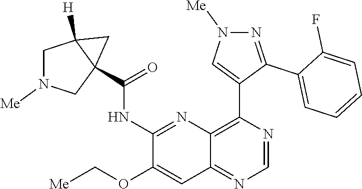 [SMILES: CCOc1cc2ncnc(-c3cn(C)nc3-c3ccccc3F)c2nc1NC(=O)[C@]12C[C@H]1CN(C)C2] |
| | or |
| | 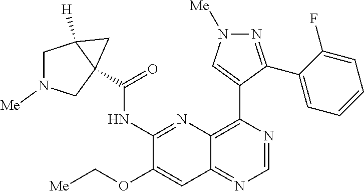 [SMILES: CCOc1cc2ncnc(-c3cn(C)nc3-c3ccccc3F)c2nc1NC(=O)[C@@]12C[C@@H]1CN(C)C2] |
| 402 | 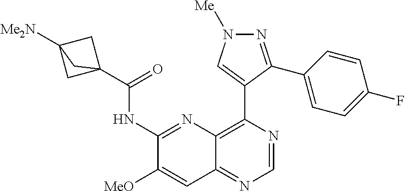 [SMILES: COc1cc2ncnc(-c3cn(C)nc3-c3ccc(F)cc3)c2nc1NC(=O)C12CC(N(C)C)(C1)C2] |
| 403 | 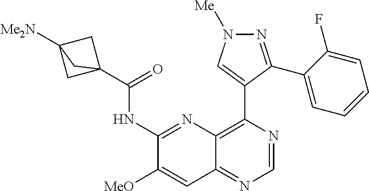 [SMILES: COc1cc2ncnc(-c3cn(C)nc3-c3ccccc3F)c2nc1NC(=O)C12CC(N(C)C)(C1)C2] |
| 404 | 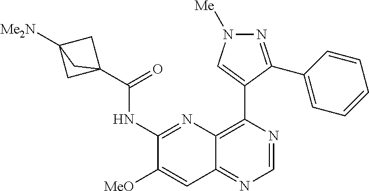 [SMILES: COc1cc2ncnc(-c3cn(C)nc3-c3ccccc3)c2nc1NC(=O)C12CC(N(C)C)(C1)C2] |
| 405 | 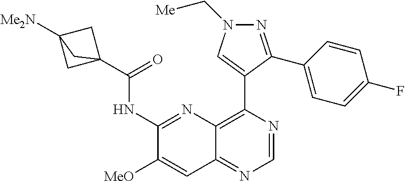 [SMILES: CCn1cc(-c2ncnc3cc(OC)c(NC(=O)C45CC(N(C)C)(C4)C5)nc23)c(-c2ccc(F)cc2)n1] |
| 406 | 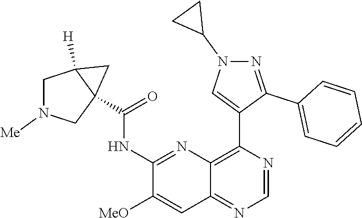 [SMILES: COc1cc2ncnc(-c3cn(C4CC4)nc3-c3ccccc3)c2nc1NC(=O)[C@@]12C[C@@H]1CN(C)C2] |
| | or |
| | 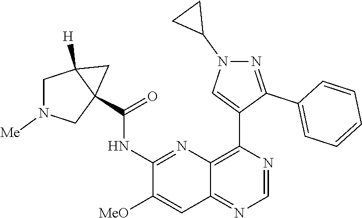 [SMILES: COc1cc2ncnc(-c3cn(C4CC4)nc3-c3ccccc3)c2nc1NC(=O)[C@]12C[C@H]1CN(C)C2] |
| 407 | 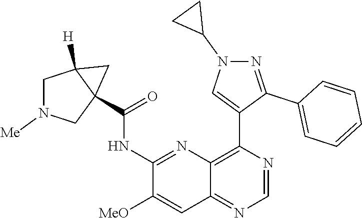 [SMILES: COc1cc2ncnc(-c3cn(C4CC4)nc3-c3ccccc3)c2nc1NC(=O)[C@]12C[C@H]1CN(C)C2] |
| | or |
| | 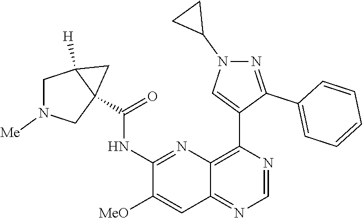 [SMILES: COc1cc2ncnc(-c3cn(C4CC4)nc3-c3ccccc3)c2nc1NC(=O)[C@@]12C[C@@H]1CN(C)C2] |
| 408 | 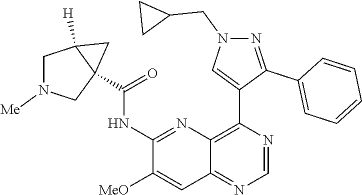 [SMILES: COc1cc2ncnc(-c3cn(CC4CC4)nc3-c3ccccc3)c2nc1NC(=O)[C@@]12C[C@@H]1CN(C)C2] |
| | or |
| | 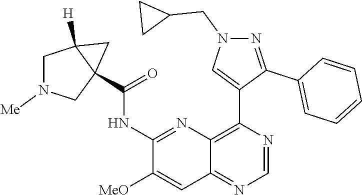 [SMILES: COc1cc2ncnc(-c3cn(CC4CC4)nc3-c3ccccc3)c2nc1NC(=O)[C@]12C[C@H]1CN(C)C2] |
| 409 | 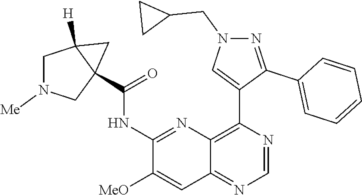 [SMILES: COc1cc2ncnc(-c3cn(CC4CC4)nc3-c3ccccc3)c2nc1NC(=O)[C@]12C[C@H]1CN(C)C2] |
| | or |
| | 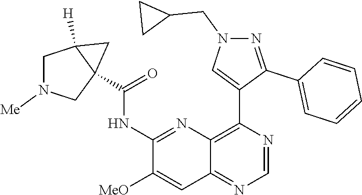 [SMILES: COc1cc2ncnc(-c3cn(CC4CC4)nc3-c3ccccc3)c2nc1NC(=O)[C@@]12C[C@@H]1CN(C)C2] |
| 410 | 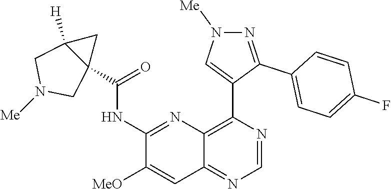 [SMILES: COc1cc2ncnc(-c3cn(C)nc3-c3ccc(F)cc3)c2nc1NC(=O)[C@@]12C[C@@H]1CN(C)C2] |
| | or |
| | 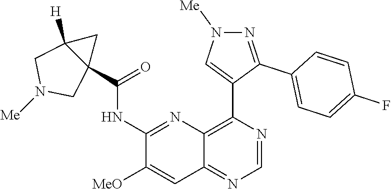 [SMILES: COc1cc2ncnc(-c3cn(C)nc3-c3ccc(F)cc3)c2nc1NC(=O)[C@]12C[C@H]1CN(C)C2] |
| 411 | 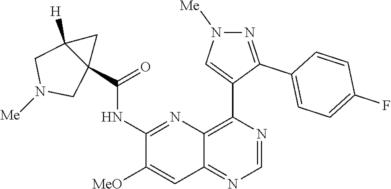 [SMILES: COc1cc2ncnc(-c3cn(C)nc3-c3ccc(F)cc3)c2nc1NC(=O)[C@]12C[C@H]1CN(C)C2] |
| | or |
| | 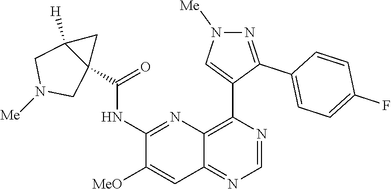 [SMILES: COc1cc2ncnc(-c3cn(C)nc3-c3ccc(F)cc3)c2nc1NC(=O)[C@@]12C[C@@H]1CN(C)C2] |
| 412 | 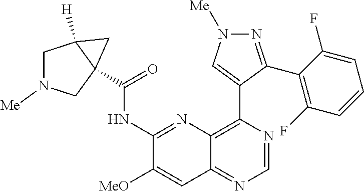 [SMILES: COc1cc2ncnc(-c3cn(C)nc3-c3c(F)cccc3F)c2nc1NC(=O)[C@@]12C[C@@H]1CN(C)C2] |
| | or |
| | 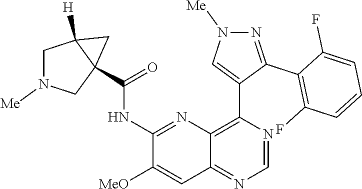 [SMILES: COc1cc2ncnc(-c3cn(C)nc3-c3c(F)cccc3F)c2nc1NC(=O)[C@]12C[C@H]1CN(C)C2] |
| 413 | 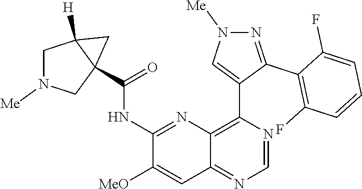 [SMILES: COc1cc2ncnc(-c3cn(C)nc3-c3c(F)cccc3F)c2nc1NC(=O)[C@]12C[C@H]1CN(C)C2] |
| | or |
| | 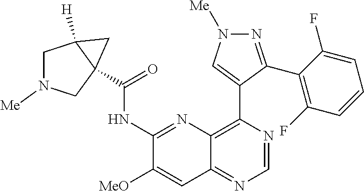 [SMILES: COc1cc2ncnc(-c3cn(C)nc3-c3c(F)cccc3F)c2nc1NC(=O)[C@@]12C[C@@H]1CN(C)C2] |
| 414 | 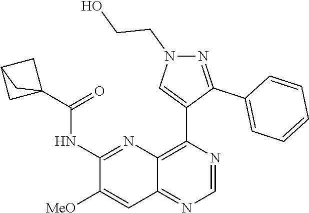 [SMILES: COc1cc2ncnc(-c3cn(CCO)nc3-c3ccccc3)c2nc1NC(=O)C12CC(C1)C2] |
| 415 | 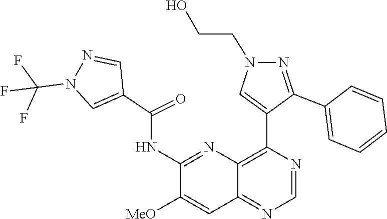 [SMILES: COc1cc2ncnc(-c3cn(CCO)nc3-c3ccccc3)c2nc1NC(=O)c1cnn(C(F)(F)F)c1] |
| 416 | 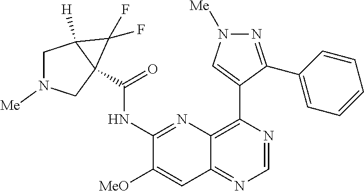 [SMILES: COc1cc2ncnc(-c3cn(C)nc3-c3ccccc3)c2nc1NC(=O)[C@]12CN(C)C[C@H]1C2(F)F] |
| | or |
| | 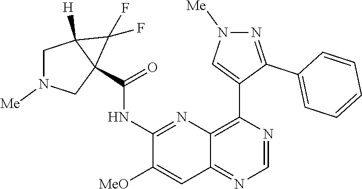 [SMILES: COc1cc2ncnc(-c3cn(C)nc3-c3ccccc3)c2nc1NC(=O)[C@@]12CN(C)C[C@@H]1C2(F)F] |
| 417 | 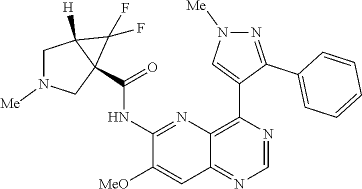 [SMILES: COc1cc2ncnc(-c3cn(C)nc3-c3ccccc3)c2nc1NC(=O)[C@@]12CN(C)C[C@@H]1C2(F)F] |
| | or |
| | 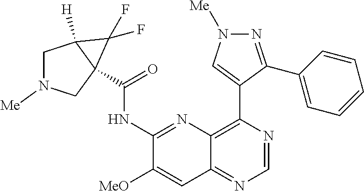 [SMILES: COc1cc2ncnc(-c3cn(C)nc3-c3ccccc3)c2nc1NC(=O)[C@]12CN(C)C[C@H]1C2(F)F] |
| 418 | 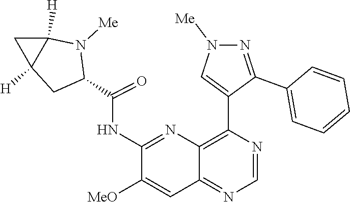 [SMILES: COc1cc2ncnc(-c3cn(C)nc3-c3ccccc3)c2nc1NC(=O)[C@@H]1C[C@H]2C[C@H]2N1C] |
| 419 | 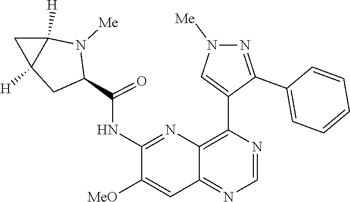 [SMILES: COc1cc2ncnc(-c3cn(C)nc3-c3ccccc3)c2nc1NC(=O)[C@H]1C[C@H]2C[C@H]2N1C] |
| 420 | 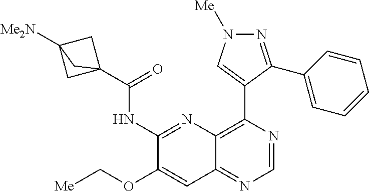 [SMILES: CCOc1cc2ncnc(-c3cn(C)nc3-c3ccccc3)c2nc1NC(=O)C12CC(N(C)C)(C1)C2] |
| 421 | 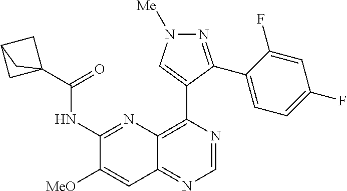 [SMILES: COc1cc2ncnc(-c3cn(C)nc3-c3ccc(F)cc3F)c2nc1NC(=O)C12CC(C1)C2] |
| 422 | 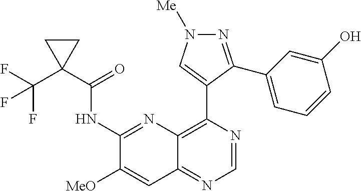 [SMILES: COc1cc2ncnc(-c3cn(C)nc3-c3cccc(O)c3)c2nc1NC(=O)C1(C(F)(F)F)CC1] |
| 423 | 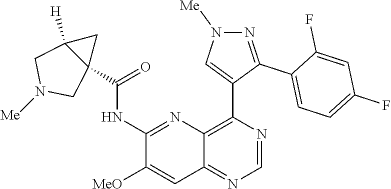 [SMILES: COc1cc2ncnc(-c3cn(C)nc3-c3ccc(F)cc3F)c2nc1NC(=O)[C@@]12C[C@@H]1CN(C)C2] |
| | or |
| | 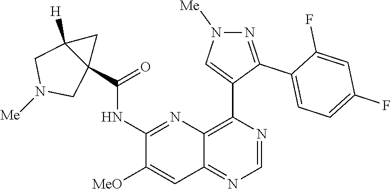 [SMILES: COc1cc2ncnc(-c3cn(C)nc3-c3ccc(F)cc3F)c2nc1NC(=O)[C@]12C[C@H]1CN(C)C2] |
| 424 | 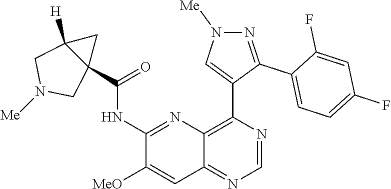 [SMILES: COc1cc2ncnc(-c3cn(C)nc3-c3ccc(F)cc3F)c2nc1NC(=O)[C@]12C[C@H]1CN(C)C2] |
| | or |
| | 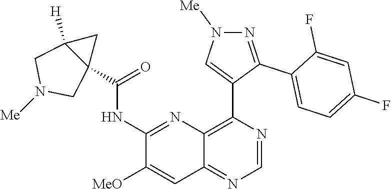 [SMILES: COc1cc2ncnc(-c3cn(C)nc3-c3ccc(F)cc3F)c2nc1NC(=O)[C@@]12C[C@@H]1CN(C)C2] |
| 425 | 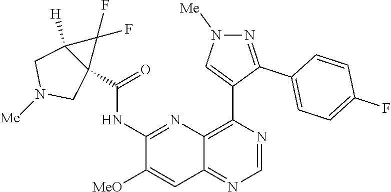 [SMILES: COc1cc2ncnc(-c3cn(C)nc3-c3ccc(F)cc3)c2nc1NC(=O)[C@]12CN(C)C[C@H]1C2(F)F] |
| | or |
| | 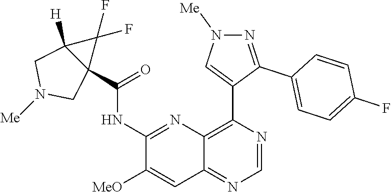 [SMILES: COc1cc2ncnc(-c3cn(C)nc3-c3ccc(F)cc3)c2nc1NC(=O)[C@@]12CN(C)C[C@@H]1C2(F)F] |
| 426 | 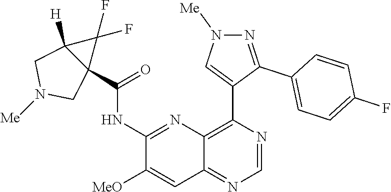 [SMILES: COc1cc2ncnc(-c3cn(C)nc3-c3ccc(F)cc3)c2nc1NC(=O)[C@@]12CN(C)C[C@@H]1C2(F)F] |
| | or |
| | 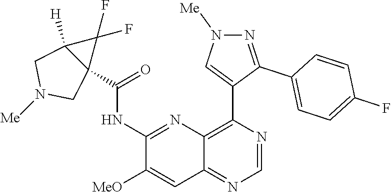 [SMILES: COc1cc2ncnc(-c3cn(C)nc3-c3ccc(F)cc3)c2nc1NC(=O)[C@]12CN(C)C[C@H]1C2(F)F] |
| 427 | 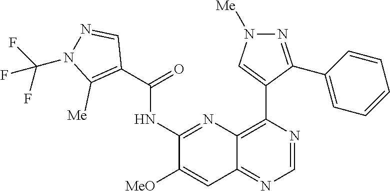 [SMILES: COc1cc2ncnc(-c3cn(C)nc3-c3ccccc3)c2nc1NC(=O)c1cnn(C(F)(F)F)c1C] |
| 428 | 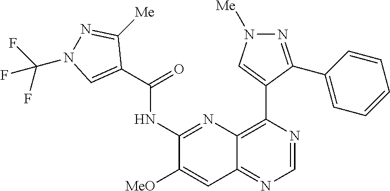 [SMILES: COc1cc2ncnc(-c3cn(C)nc3-c3ccccc3)c2nc1NC(=O)c1cn(C(F)(F)F)nc1C] |
| 429 | 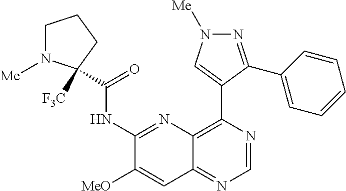 [SMILES: COc1cc2ncnc(-c3cn(C)nc3-c3ccccc3)c2nc1NC(=O)[C@@]1(C(F)(F)F)CCCN1C] |
| | or |
| | 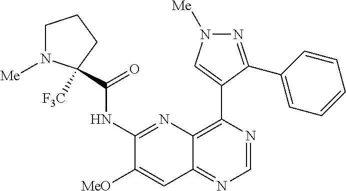 [SMILES: COc1cc2ncnc(-c3cn(C)nc3-c3ccccc3)c2nc1NC(=O)[C@]1(C(F)(F)F)CCCN1C] |
| 430 | 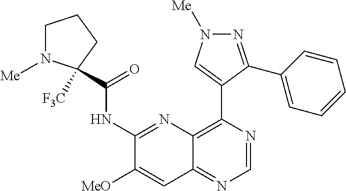 [SMILES: COc1cc2ncnc(-c3cn(C)nc3-c3ccccc3)c2nc1NC(=O)[C@]1(C(F)(F)F)CCCN1C] |
| | or |
| | 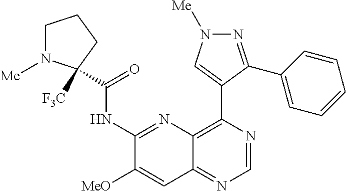 [SMILES: COc1cc2ncnc(-c3cn(C)nc3-c3ccccc3)c2nc1NC(=O)[C@@]1(C(F)(F)F)CCCN1C] |
| 431 | 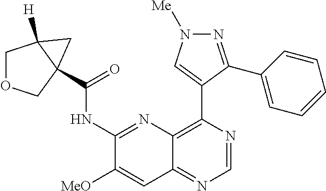 [SMILES: COc1cc2ncnc(-c3cn(C)nc3-c3ccccc3)c2nc1NC(=O)[C@@]12COC[C@@H]1C2] |
| | or |
| | 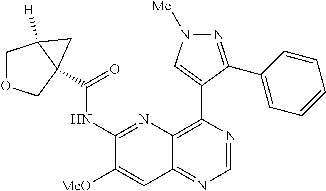 [SMILES: COc1cc2ncnc(-c3cn(C)nc3-c3ccccc3)c2nc1NC(=O)[C@]12COC[C@H]1C2] |
| 432 | 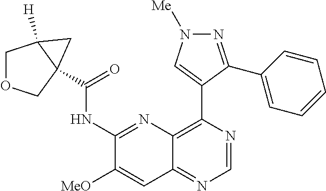 [SMILES: COc1cc2ncnc(-c3cn(C)nc3-c3ccccc3)c2nc1NC(=O)[C@]12COC[C@H]1C2] |
| | or |
| | 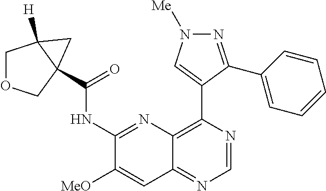 [SMILES: COc1cc2ncnc(-c3cn(C)nc3-c3ccccc3)c2nc1NC(=O)[C@@]12COC[C@@H]1C2] |
| 433 | 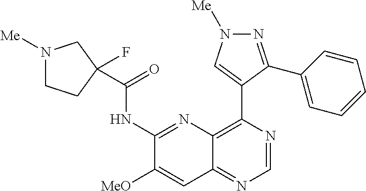 [SMILES: COc1cc2ncnc(-c3cn(C)nc3-c3ccccc3)c2nc1NC(=O)C1(F)CCN(C)C1] |
| 434 | 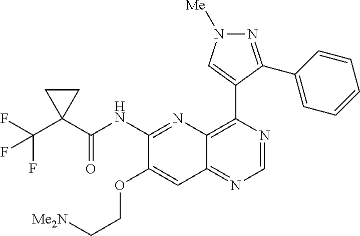 [SMILES: CN(C)CCOc1cc2ncnc(-c3cn(C)nc3-c3ccccc3)c2nc1NC(=O)C1(C(F)(F)F)CC1] |
| 435 | 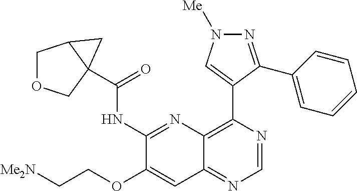 [SMILES: CN(C)CCOc1cc2ncnc(-c3cn(C)nc3-c3ccccc3)c2nc1NC(=O)C12COCC1C2] |
| 436 | 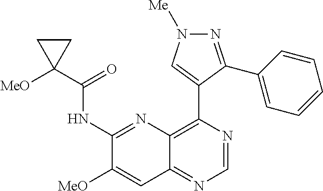 [SMILES: COc1cc2ncnc(-c3cn(C)nc3-c3ccccc3)c2nc1NC(=O)C1(OC)CC1] |
| 437 | 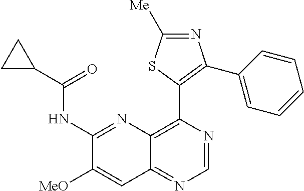 [SMILES: COc1cc2ncnc(-c3sc(C)nc3-c3ccccc3)c2nc1NC(=O)C1CC1] |
| 438 | 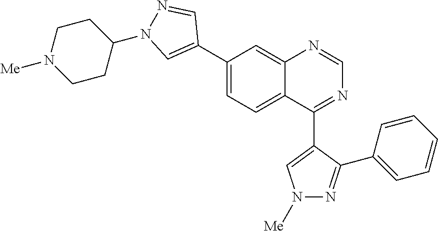 [SMILES: CN1CCC(n2cc(-c3ccc4c(-c5cn(C)nc5-c5ccccc5)ncnc4c3)cn2)CC1] |
| 439 | 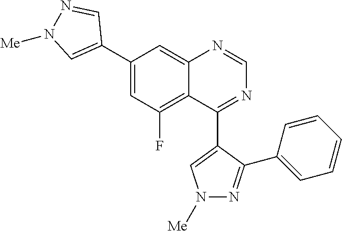 [SMILES: Cn1cc(-c2cc(F)c3c(-c4cn(C)nc4-c4ccccc4)ncnc3c2)cn1] |
| 440 | 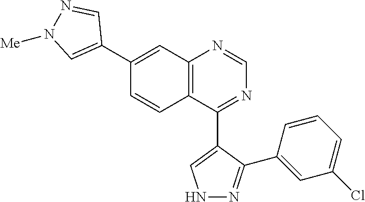 [SMILES: Cn1cc(-c2ccc3c(-c4c[nH]nc4-c4cccc(Cl)c4)ncnc3c2)cn1] |
| 441 | 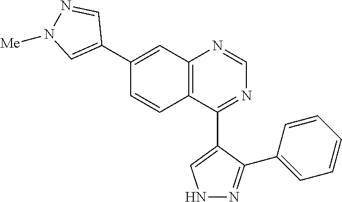 [SMILES: Cn1cc(-c2ccc3c(-c4c[nH]nc4-c4ccccc4)ncnc3c2)cn1] |
| 442 | 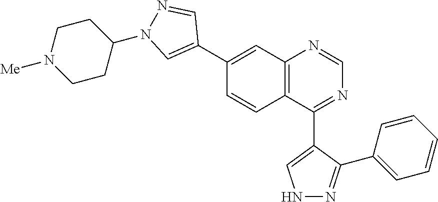 [SMILES: CN1CCC(n2cc(-c3ccc4c(-c5c[nH]nc5-c5ccccc5)ncnc4c3)cn2)CC1] |
| 443 | 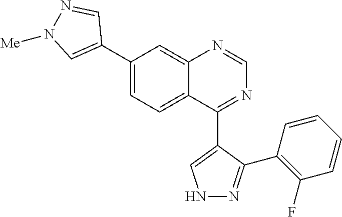 [SMILES: Cn1cc(-c2ccc3c(-c4c[nH]nc4-c4ccccc4F)ncnc3c2)cn1] |
| 444 | 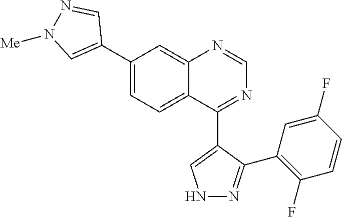 [SMILES: Cn1cc(-c2ccc3c(-c4c[nH]nc4-c4cc(F)ccc4F)ncnc3c2)cn1] |
| 445 | 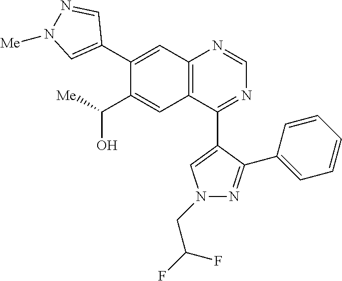 [SMILES: C[C@@H](O)c1cc2c(-c3cn(CC(F)F)nc3-c3ccccc3)ncnc2cc1-c1cnn(C)c1] |
| | or |
| | 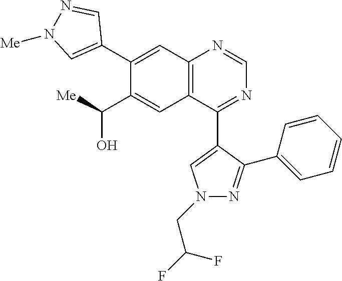 [SMILES: C[C@H](O)c1cc2c(-c3cn(CC(F)F)nc3-c3ccccc3)ncnc2cc1-c1cnn(C)c1] |
| 446 | 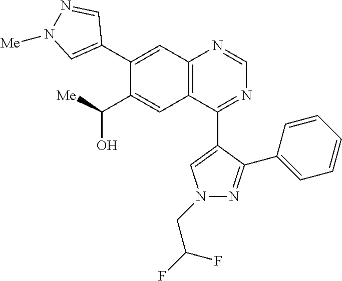 [SMILES: C[C@H](O)c1cc2c(-c3cn(CC(F)F)nc3-c3ccccc3)ncnc2cc1-c1cnn(C)c1] |
| | or |
| | 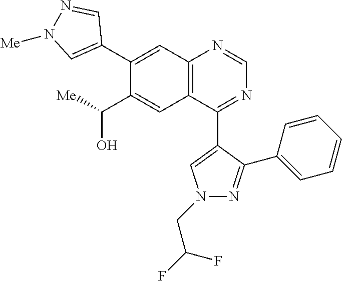 [SMILES: C[C@@H](O)c1cc2c(-c3cn(CC(F)F)nc3-c3ccccc3)ncnc2cc1-c1cnn(C)c1] |
| 447 | 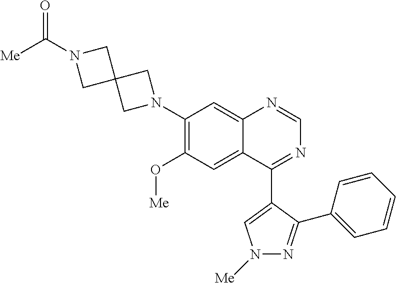 [SMILES: COc1cc2c(-c3cn(C)nc3-c3ccccc3)ncnc2cc1N1CC2(CN(C(C)=O)C2)C1] |
| 448 | 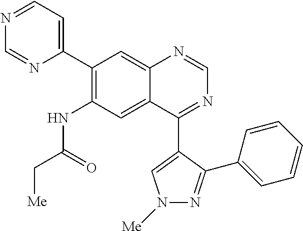 [SMILES: CCC(=O)Nc1cc2c(-c3cn(C)nc3-c3ccccc3)ncnc2cc1-c1ccncn1] |
| 449 | 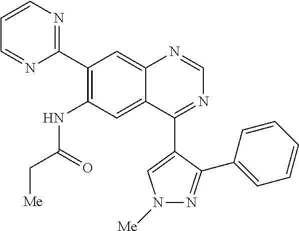 [SMILES: CCC(=O)Nc1cc2c(-c3cn(C)nc3-c3ccccc3)ncnc2cc1-c1ncccn1] |
| 450 | 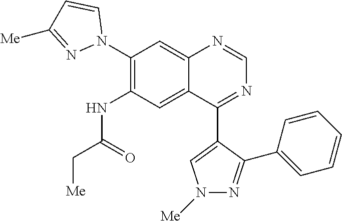 [SMILES: CCC(=O)Nc1cc2c(-c3cn(C)nc3-c3ccccc3)ncnc2cc1-n1ccc(C)n1] |
| 451 | 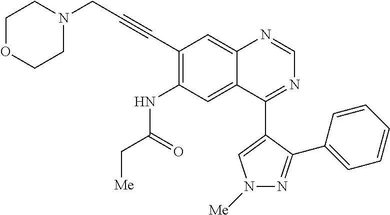 [SMILES: CCC(=O)Nc1cc2c(-c3cn(C)nc3-c3ccccc3)ncnc2cc1C#CCN1CCOCC1] |
| 452 | 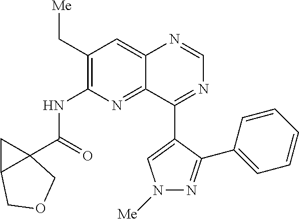 [SMILES: CCc1cc2ncnc(-c3cn(C)nc3-c3ccccc3)c2nc1NC(=O)C12COCC1C2] |
| 453 | 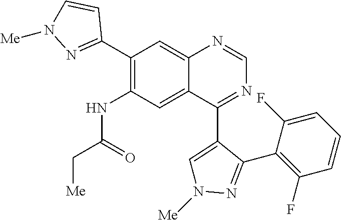 [SMILES: CCC(=O)Nc1cc2c(-c3cn(C)nc3-c3c(F)cccc3F)ncnc2cc1-c1ccn(C)n1] |
| 454 | 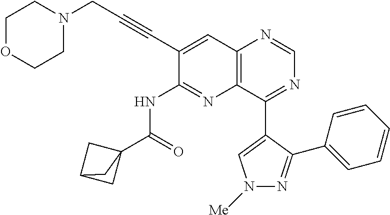 [SMILES: Cn1cc(-c2ncnc3cc(C#CCN4CCOCC4)c(NC(=O)C45CC(C4)C5)nc23)c(-c2ccccc2)n1] |
| 458 | 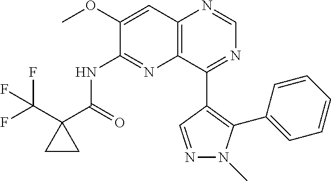 [SMILES: COc1cc2ncnc(-c3cnn(C)c3-c3ccccc3)c2nc1NC(=O)C1(C(F)(F)F)CC1] |
| 459 | 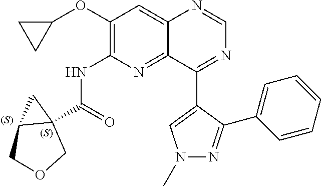 |
| 460 | 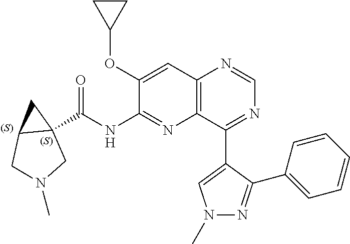 |
| 461 | 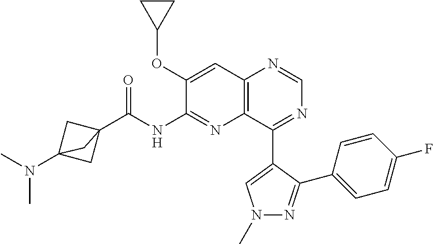 [SMILES: CN(C)C12CC(C(=O)Nc3nc4c(-c5cn(C)nc5-c5ccc(F)cc5)ncnc4cc3OC3CC3)(C1)C2] |
| 463 | 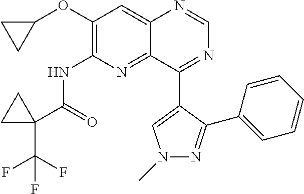 [SMILES: Cn1cc(-c2ncnc3cc(OC4CC4)c(NC(=O)C4(C(F)(F)F)CC4)nc23)c(-c2ccccc2)n1] |
| 464 | 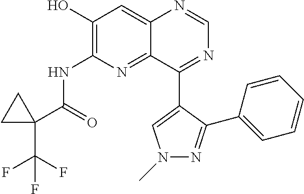 [SMILES: Cn1cc(-c2ncnc3cc(O)c(NC(=O)C4(C(F)(F)F)CC4)nc23)c(-c2ccccc2)n1] |
| 465 | 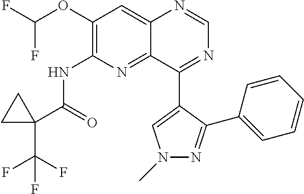 [SMILES: Cn1cc(-c2ncnc3cc(OC(F)F)c(NC(=O)C4(C(F)(F)F)CC4)nc23)c(-c2ccccc2)n1] |
| 466 | 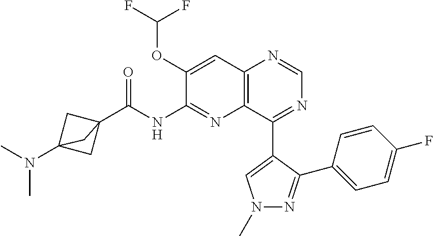 [SMILES: CN(C)C12CC(C(=O)Nc3nc4c(-c5cn(C)nc5-c5ccc(F)cc5)ncnc4cc3OC(F)F)(C1)C2] |
| 467 | 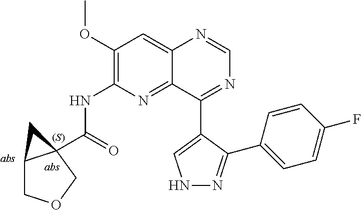 |
| 468 | 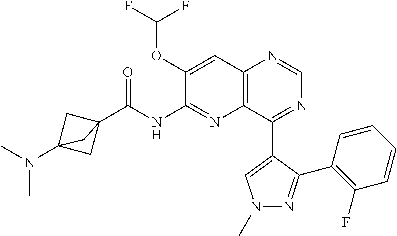 [SMILES: CN(C)C12CC(C(=O)Nc3nc4c(-c5cn(C)nc5-c5ccccc5F)ncnc4cc3OC(F)F)(C1)C2] |
| 469 | 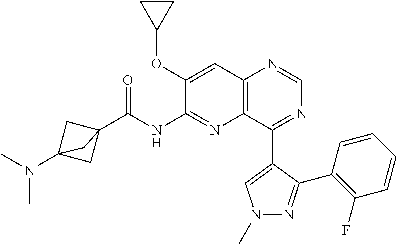 [SMILES: CN(C)C12CC(C(=O)Nc3nc4c(-c5cn(C)nc5-c5ccccc5F)ncnc4cc3OC3CC3)(C1)C2] |
| 470 | 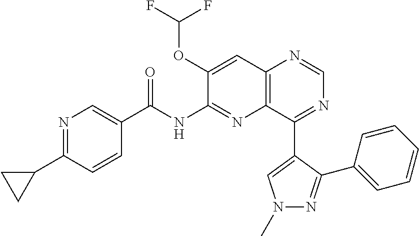 [SMILES: Cn1cc(-c2ncnc3cc(OC(F)F)c(NC(=O)c4ccc(C5CC5)nc4)nc23)c(-c2ccccc2)n1] |
| 472 | 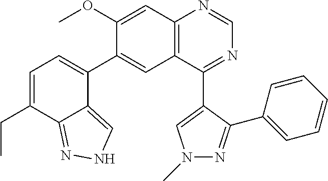 [SMILES: CCc1ccc(-c2cc3c(-c4cn(C)nc4-c4ccccc4)ncnc3cc2OC)c2c[nH]nc12] |
| 474 | 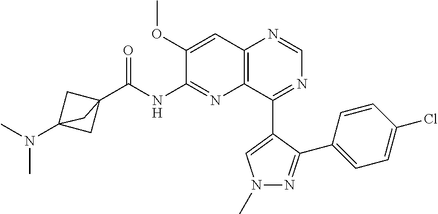 [SMILES: COc1cc2ncnc(-c3cn(C)nc3-c3ccc(Cl)cc3)c2nc1NC(=O)C12CC(N(C)C)(C1)C2] |
| 475 | 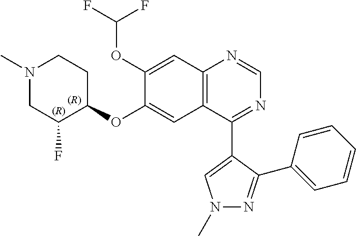 |
| 476 | 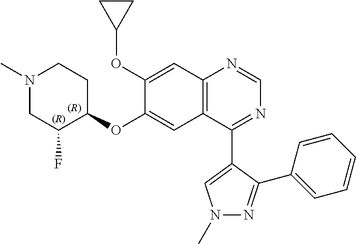 |
| 477 | 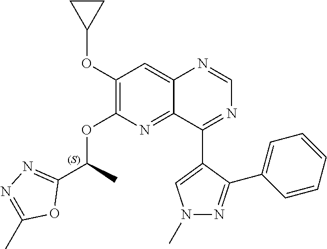 |
| | or |
| | 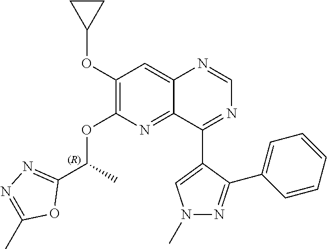 |
| 478 | 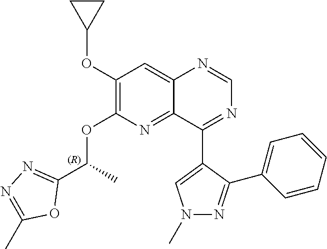 |
| | or |
| | 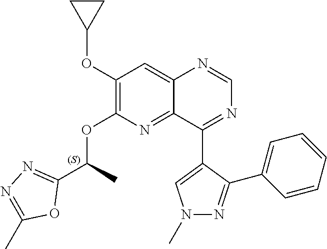 |
| 479 | 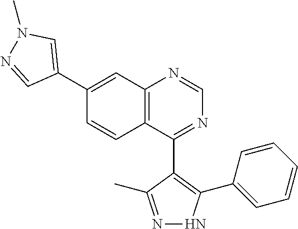 [SMILES: Cc1n[nH]c(-c2ccccc2)c1-c1ncnc2cc(-c3cnn(C)c3)ccc12] |
| 480 | 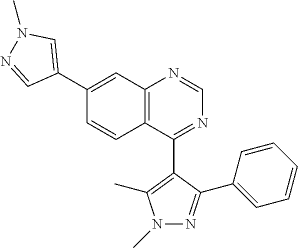 [SMILES: Cc1c(-c2ncnc3cc(-c4cnn(C)c4)ccc23)c(-c2ccccc2)nn1C] |
| 481 | 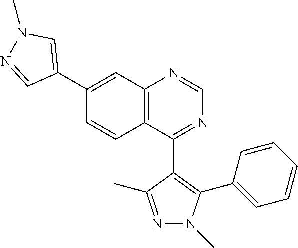 [SMILES: Cc1nn(C)c(-c2ccccc2)c1-c1ncnc2cc(-c3cnn(C)c3)ccc12] |
| 482 | 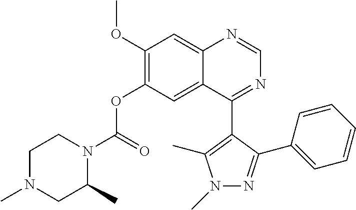 [SMILES: COc1cc2ncnc(-c3c(-c4ccccc4)nn(C)c3C)c2cc1OC(=O)N1CCN(C)C[C@@H]1C] |
| 483 | 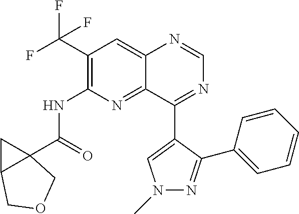 [SMILES: Cn1cc(-c2ncnc3cc(C(F)(F)F)c(NC(=O)C45COCC4C5)nc23)c(-c2ccccc2)n1] |
| 484 | 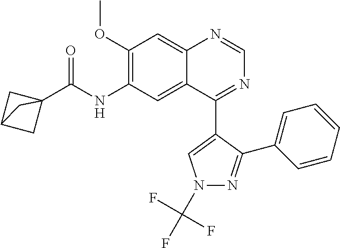 [SMILES: COc1cc2ncnc(-c3cn(C(F)(F)F)nc3-c3ccccc3)c2cc1NC(=O)C12CC(C1)C2] |
| 485 | 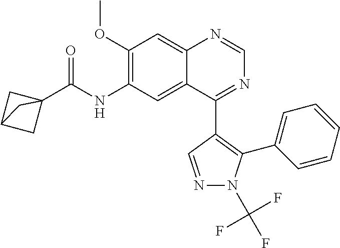 [SMILES: COc1cc2ncnc(-c3cnn(C(F)(F)F)c3-c3ccccc3)c2cc1NC(=O)C12CC(C1)C2] |
| 486 | 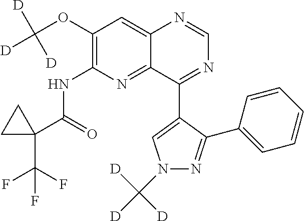 [SMILES: [2H]C([2H])([2H])Oc1cc2ncnc(-c3cn(C([2H])([2H])[2H])nc3-c3ccccc3)c2nc1NC(=O)C1(C(F)(F)F)CC1] |
| 487 | 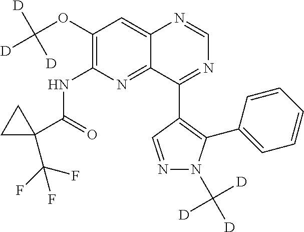 [SMILES: [2H]C([2H])([2H])Oc1cc2ncnc(-c3cnn(C([2H])([2H])[2H])c3-c3ccccc3)c2nc1NC(=O)C1(C(F)(F)F)CC1] |
| 488 | 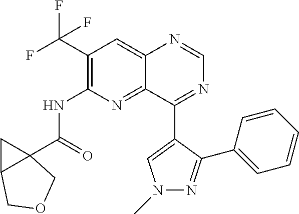 [SMILES: Cn1cc(-c2ncnc3cc(C(F)(F)F)c(NC(=O)C45COCC4C5)nc23)c(-c2ccccc2)n1] |
| 489 | 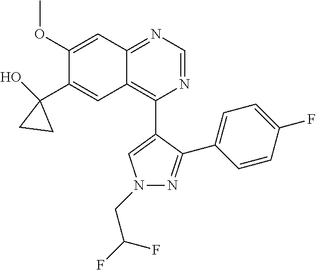 [SMILES: COc1cc2ncnc(-c3cn(CC(F)F)nc3-c3ccc(F)cc3)c2cc1C1(O)CC1] |
| 490 | 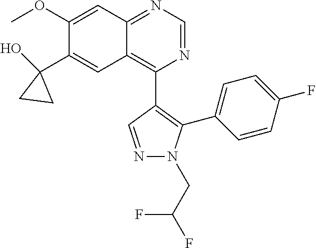 [SMILES: COc1cc2ncnc(-c3cnn(CC(F)F)c3-c3ccc(F)cc3)c2cc1C1(O)CC1] |
| 492 | 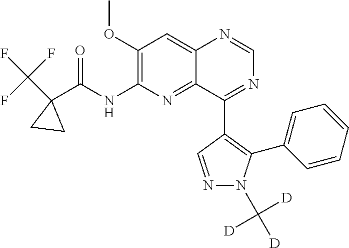 [SMILES: [2H]C([2H])([2H])n1ncc(-c2ncnc3cc(OC)c(NC(=O)C4(C(F)(F)F)CC4)nc23)c1-c1ccccc1] |
| 493 | 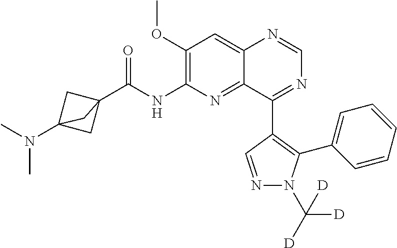 [SMILES: [2H]C([2H])([2H])n1ncc(-c2ncnc3cc(OC)c(NC(=O)C45CC(N(C)C)(C4)C5)nc23)c1-c1ccccc1] |
| 494 | 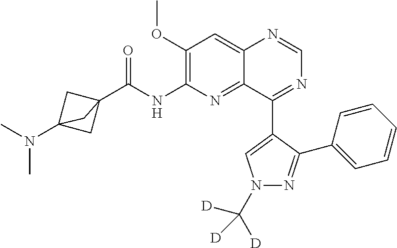 [SMILES: [2H]C([2H])([2H])n1cc(-c2ncnc3cc(OC)c(NC(=O)C45CC(N(C)C)(C4)C5)nc23)c(-c2ccccc2)n1] |
| 495 | 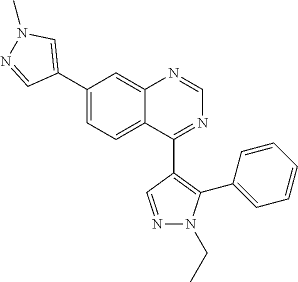 [SMILES: CCn1ncc(-c2ncnc3cc(-c4cnn(C)c4)ccc23)c1-c1ccccc1] |
| 496 | 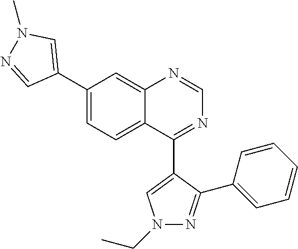 [SMILES: CCn1cc(-c2ncnc3cc(-c4cnn(C)c4)ccc23)c(-c2ccccc2)n1] |
| 497 | 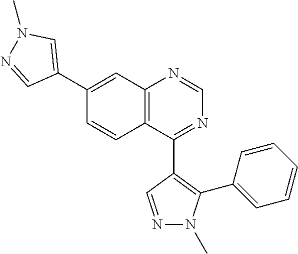 [SMILES: Cn1cc(-c2ccc3c(-c4cnn(C)c4-c4ccccc4)ncnc3c2)cn1] |
| 498 | 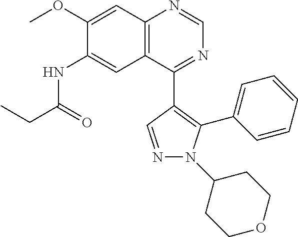 [SMILES: CCC(=O)Nc1cc2c(-c3cnn(C4CCOCC4)c3-c3ccccc3)ncnc2cc1OC] |
| 499 | 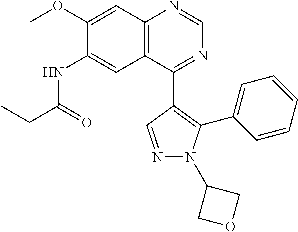 [SMILES: CCC(=O)Nc1cc2c(-c3cnn(C4COC4)c3-c3ccccc3)ncnc2cc1OC] |
| 500 | 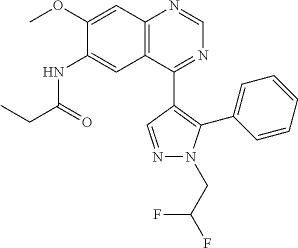 [SMILES: CCC(=O)Nc1cc2c(-c3cnn(CC(F)F)c3-c3ccccc3)ncnc2cc1OC] |
| Preparation 190 | 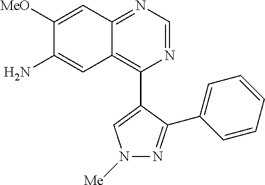 [SMILES: COc1cc2ncnc(-c3cn(C)nc3-c3ccccc3)c2cc1N] |

In one embodiment, the compounds of Examples 455-457 and 458A in Table 3, and pharmaceutically acceptable salts thereof, are excluded from the disclosure.

In some embodiments, the present disclosure provides a compound according to structural formula (A), (B), (C), (I), (II-1), (II-1a), (II-2), (II-3), (II-3a), (II-3b), (II-3c), (II-4), (II-4a), (II-5), (II-5a), (II-6), (II-7), (II-7a), (II-7b), (II-8), (II-8a), (II-9), (II-9a), (II-9b), (II-10), (II-11), (II-11a), (II-11b), (II-12), (II-12a), (III), (III-1) or (III-2), or any one of the compounds of disclosed in the examples (including intermediates) and Table 1, or a pharmaceutically acceptable salt thereof, wherein one or more hydrogen is replaced with deuterium.

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

When two stereoisomers are depicted by their chemical names or structures, and the names or structures are connected by an “or”, one or the other of the two stereoisomers is intended, but not both.

A racemic mixture means a mixture of 50% of one enantiomer and 50% of its corresponding enantiomer. The present teachings encompass all enantiomerically-pure, enantiomerically-enriched, diastereomerically pure, diastereomerically-enriched, and racemic mixtures, and diastereomeric mixtures of the compounds disclosed herein.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well-known asymmetric synthetic methods.

“First eluting compound” or “Peak 1” in the Experimental section refers to an intended reaction product compound obtained from a chromatography separation/purification that elutes earlier than a second intended reaction product compound from the same preceding reaction. The second intended product compound is referred to as “Second eluting compound” or “Peak 2”.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are selective EGFR inhibitors. As used herein, the term “selective EGFR inhibitor” means a compound which selectively inhibits certain mutant EGFR kinases over wild-type EGFR and the kinome. Said another way, a selective EGFR inhibitor has no or low activity against wild-type EGFR and the kinome. A selective EGFR inhibitor's inhibitory activity against certain mutant EGFR kinases is more potent in terms of IC₅₀ value (i.e., the IC₅₀ value is subnanomolar) when compared with its inhibitory activity against wild-type EGFR and many other kinases. Potency can be measured using known biochemical assays.

Some compounds of the disclosure have the advantage of good penetration of the brain. The ability of a particular compound to cross the BBB and penetrate the brain can be assessed using a variety of known methods or combinations of such methods. One in vitro method that is frequently used to predict a compound's in vivo brain penetration is P-gp efflux ratio. P-glycoprotein (P-gp) is expressed at the blood-brain barrier (BBB) and restricts the penetration of its substrates into the central nervous system (CNS). Compounds that are found to be good P-gp substrates in vitro (i.e., have a high efflux ratio) are predicted to have poor in vivo brain penetration. In order to measure the P-gp efflux ratio. Madin-Darby canine kidney cells overexpressing P-gp (MDCK-MDR1 cells) the apparent apical to basolateral permeability (Papp[A-B]) and the apparent basolateral to apical permeability (Papp[B-A]) for compounds is determined. The P-gp efflux ratio is a measure of the ratio of Papp[B-A]/Papp[A-B]. In some embodiments, a compound of the disclosure has a P-gp efflux ratio of less than 2, less than 3, less than 4, less than 5.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes. Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (A) or (I)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The present disclosure provides a method of inhibiting certain mutant forms of epidermal growth factor receptor (EGFR) in a subject in need thereof, comprising administering to the subject an effective amount of a compound disclosed herein, a pharmaceutically acceptable salt thereof or a pharmaceutical composition disclosed herein. Mutant forms of EGFR include for example, EGFR with LRCS mutation (the exon 19 deletion (del19) or exon 21 (L858R) substitution mutation, and C797X (e.g., C797S) mutation). Subjects “in need of inhibiting EGFR” are those having a disease for which a beneficial therapeutic effect can be achieved by inhibiting at least one mutant EGFR, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by mutant EGFR, wherein the inhibition of the mutant EGFR is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. Cancers to be treated according to the disclosed methods include lung cancer, colon cancer, urothelial cancer, breast cancer, prostate cancer, brain cancers, ovarian cancer, gastric cancer, pancreatic cancer, head and neck cancer, bladder cancer, and mesothelioma, including metastasis (in particular brain metastasis) of all cancers listed. Typically, the cancer is characterized by at one or more EGFR mutations described herein. In a specific embodiment, the cancer has progressed on or after EGFR tyrosine kinase inhibitor (TKI) therapy. In a specific embodiment, the disease has progressed on or after first line 3^{rd} generation TKI, e.g. osimertinib. In a specific embodiment, the cancer was not previously treated.

In a specific embodiment, the cancer to be treated is lung cancer. In a more specific embodiment, the cancer is non-small cell lung cancer (NSCLC). In some embodiments, the lung cancer is locally advanced or metastatic NSCLC, NSCLC adenocarcinoma, NSCLC with squamous histology and NSCLC with non-squamous histology. In another embodiment, the lung cancer is NSCLC adenocarcinoma. In another specific embodiment, the lung cancer (or non-small cell lung cancer) has metastasized to the brain.

In another embodiment, the disease/condition/or cancer associated with or modulated by mutant EGFR that is characterized by an EGFR genotype selected from genotypes 1-36 according the Table below (del18=Exon 18 deletion, specifically, e.g., del E709_T710 insD; and del19=Exon 19 deletion, specifically, e.g., delE746_A750 (most common), delE746_S752insV, del747_A750insP, delL747_P753insS, and delS752_1759; ex20ins-Exon 20 insertion, specifically, e.g., D761-E762insX, A763-Y764insX, Y764-V765insX, V765-M766insX, A767-S768insX, S768-D769insX, V769-D770insX, N771-P772insX, P772-H773insX, H773-V774insX, and V774-C775insX):

EGFR Genotype

| | |
|---|---|
| 1 | EGFR del19 |
| 2 | EGFR del19 C797S |
| 3 | EGFR del19 C797X (C797G or C797N or C797Y or C797T or |
| | C797D) |
| 4 | EGFR del19 L792X (L792F, L792H or L792Y) |
| 5 | EGFR del19 G796R (G796S) |
| 6 | EGFR del19 L792R (L792V or L792P) |
| 7 | EGFR del19 L718Q (L718V) |
| 8 | EGFR del19 G724S |
| 9 | EGFR del19 S768I (SV768IL) |
| 10 | EGFR del 19 V834L |
| 11 | EGFR del19 C797S L718Q (L718V) |
| 12 | EGFR del19 L718Q (L718V) A750P |
| 13 | EGFR L858R |
| 14 | EGFR L858R C797S |
| 15 | EGFR L858R C797X (797G or C797N or C797Y or C797T or |
| | C797D) |
| 16 | EGFR L858R L792X (L792F, L792H or L792Y) |
| 17 | EGFR L858R G796R (G796S) |
| 18 | EGFR L858R L792R (L792V or L792P) |
| 19 | EGFR L858R L718Q (L718V) |
| 20 | EGFR L858R G724S |
| 21 | EGFR L858R S768I (SV768IL) |
| 22 | EGFR L858R V834L |
| 23 | EGFR L858R C797S L718Q (L718V) |
| 24 | EGFR L858R L718Q (L718V) A750P |
| 25 | EGFR L861Q |
| 27 | EGFR L861Q C797S/G/N |
| 28 | EGFR del18 |
| 29 | EGFR G719X (G719A, G719S, G719C, G719R, G719D, or G719V) |
| 30 | EGFR E709X (E709K, E709H, or E709A) |
| 31 | EGFR E709X (E709K, E709H, or E709A) (G719A, G719S, G719C, |
| | G719D, G719R, or G719V) |
| 32 | EGFR G719X (G719A, G719S, G719C, G719D, G719R, or G719V) |
| | S768I |
| 33 | EGFR S768I |
| 34 | EGFR ex20ins |
| 35 | EGFR ex20ins L718Q |
| 36 | EGFR ex20ins C797S |

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797X (C797G or C797N or C797Y or C797T or C797D).

In another embodiment, the disease/condition/or cancer (e.g., NSCLC) being treated with a disclosed compound, a pharmaceutically acceptable salt, or or a pharmaceutical composition disclosed herein is characterized by EGFR comprising EGFR del19 L792X (L792F, L792H or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 G796R (G796S).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L792R (L792V or L792P).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L718Q (L718V).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797X (797G or C797N or C797Y or C797T or C797D).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L792X (L792F, L792H or L792Y).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L718Q (L718V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del18.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719R, G719D, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A) (G719A, G719S, G719C, G719D, G719R, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719D, G719R, or G719V) S768I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR S768I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins L718Q.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR cx20ins C797S.

In another embodiment, the disease/condition/or cancer (e.g., NSCLC) being treated with a disclosed compound, a pharmaceutically acceptable salt or a pharmaceutical composition disclosed herein is characterized by an EGFR genotype selected from genotypes 1-36.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to lazertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to amivantamab.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to amivantamab and lazertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to aumolertinib (formerly almonertinib).

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to olmutinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to nazartinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to avitinib.

Another embodiment is the treatment a subject with metastatic NSCLC with tumors harboring activating Exon 19 Deletion or L858R EGFR mutations, G719X (A, S, C, D, R, V), S768I and L861Q, as well as a resistance mutation disclosed herein as detected by an approved molecular testing methodology.

Another embodiment is a disclosed compound used in combination with a 2nd or 3rd generation TKI indicated for the treatment of subject with metastatic NSCLC with tumors harboring C797X mutations as detected by an approved test, and whose disease has progressed on or after 1 or 2 prior EGFR TKI therapies.

Another embodiment is a disclosed compound for the treatment of subjects with metastatic NSCLC whose disease with on-target EGFR resistance has progressed on or after any EGFR TKI. In a specific embodiment, the disclosed compound is used in combination with a 2nd or 3rd generation TKI indicated for the treatment of subject with metastatic NSCLC.

Another embodiment is a disclosed compound for the treatment of subjects with metastatic EGFR C797X mutation-positive NSCLC as detected by an approved molecular test, whose disease has progressed on or after first-line or second-line osimertinib. In a specific embodiment, the disclosed compound is used in combination with a 2nd or 3rd generation TKI indicated for the treatment of subject with metastatic NSCLC.

In a particular embodiment, the deletions, mutations, and insertions disclosed herein are detected by an FDA-approved test.

A person of ordinary skill in the art can readily determine the certain EGFR alterations a subject possesses in a cell, cancer, gene, or gene product, e.g., whether a subject has one or more of the mutations or deletions described herein using a detection method selected from those known in the art such as hybridization-based methods, amplification-based methods, microarray analysis, flow cytometry analysis. DNA sequencing, next-generation sequencing (NGS), primer extension, PCR, in situ hybridization, fluorescent in situ hybridization, dot blot, and Southern blot.

To detect one or more EGFR deletions and/or mutations, a primary tumor sample, circulating tumor DNA (ctDNA), circulating tumor cells (CTC), and/or circulating exosomes may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples.

The compounds of the disclosure, pharmaceutically acceptable salts thereof or pharmaceutical compositions disclosed herein may be used for treating to a subject who has become refractory to treatment with one or more other EGFR inhibitors. “Refractory” means that the subject's cancer previously responded to drugs but later responds poorly or not at all. In some some embodiments, the subject has become refractory to one or more first generation EGFR inhibitors such as erlotinib, gefitinib, icotinib or lapatinib. In some embodiments, the subject has been become refractory to treatment with one or more second generation EGFR inhibitors such as afatinib, dacomitinib, poziotinib, or neratinib. In some embodiments the subject has become refractory to treatment with one or more first generation inhibitors and one or more second generation inhibitors. In some embodiments, the subject has become refractory to treatment with one or more third generation inhibitors such as osimertinib, nazartinib, or avitinib. In one embodiment, the subject has become refractory to treatment with one or more first generation EGFR inhibitors and one or more third generation EGFR inhibitors. In some embodiments, the subject has become refractory to treatment with one or more second generation EGFR inhibitors and one or more third generation EGFR inhibitors. In some embodiments, the subject has become refractory to treatment with one or more first generation inhibitors, and one or more third generation EGFR inhibitors.

### Combinations

The compounds of the disclosure, pharmaceutically acceptable salts thereof, or pharmaceutical compositions disclosed herein can be used in combination with one or more additional pharmacologically active substances. For example, the disclosure includes methods of treating a condition/disease/or cancer comprising administering to a subject in need thereof a compound of the disclosure or a pharmaceutically acceptable salt or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). For the treatment of cancer e.g., NSCLC using a compound of the disclosure or pharmaceutically acceptable salt thereof or pharmaceutical composition disclosed herein in combination with a first line therapy, for example a first, second, or third generation EGFR inhibitor (i.e., as an initial treatment before the cancer has become refractory) may forestall or delay the cancer from becoming refractory. Typically, the cancer is characterized by one of the EGFR genotypes described herein.

In one aspect, a compound of the disclosure, a pharmaceutically acceptable salt thereof or a pharmaceutical composition disclosed herein can be administered in combination with a compound disclosed in International Application Publication No. WO 2021/133809, a pharmaceutically acceptable salt thereof or a pharmaceutical composition comprising the same.

In one embodiment, a compound of the disclosure, a pharmaceutically acceptable salt thereof or a pharmaceutical composition disclosed herein can be administered in combination with a compound provided below,
(3S,4R)-3-fluoro-1-(4-(5-isopropyl-8-((2R,3S)-2-methyl-3-(methylsulfonylmethyl) azetidin-1-yl)isoquinolin-3-ylamino)pyrimidin-2-yl)-4-methylpiperidin-4-ol,
(3R,4S)-3-fluoro-1-(4-(5-isopropyl-8-((2R,3S)-2-methyl-3-(methylsulfonylmethyl) azetidin-1-yl)isoquinolin-3-ylamino)pyrimidin-2-yl)-4-methylpiperidin-4-ol,
N-(2-((3S,4R)-3-fluoro-4-methoxypiperidin-1-yl)pyrimidin-4-yl)-5-isopropyl-8-((2R,3S)-2-methyl-3-((methylsulfonyl)methyl) azetidin-1-yl)isoquinolin-3-amine,
N-(2-((3R,4S)-3-fluoro-4-methoxypiperidin-1-yl)pyrimidin-4-yl)-5-isopropyl-8-((2R,3S)-2-methyl-3-((methylsulfonyl)methyl) azetidin-1-yl)isoquinolin-3-amine,
N-(2-((3S,4R)-3-fluoro-4-(methoxy-d3) piperidin-1-yl)pyrimidin-4-yl)-5-isopropyl-8-((2R,3S)-2-methyl-3-((methylsulfonyl)methyl) azetidin-1-yl)isoquinolin-3-amine,
N-(2-((3R,4S)-3-fluoro-4-(methoxy-d3) piperidin-1-yl)pyrimidin-4-yl)-5-isopropyl-8-((2R,3S)-2-methyl-3-((methylsulfonyl)methyl) azetidin-1-yl)isoquinolin-3-amine,
(3S,4R)-1-(4-(8-((2R,3S)-3-(ethylsulfonylmethyl)-2-methylazetidin-1-yl)-5-isopropyl-2,6-naphthyridin-3-ylamino)pyrimidin-2-yl)-3-fluoro-4-methylpiperidin-4-ol,
(3S,4R)-1-(4-(8-((2R,3S)-3-(ethylsulfonylmethyl)-2-methylazetidin-1-yl)-5-isopropyl-2,7-naphthyridin-3-ylamino)pyrimidin-2-yl)-3-fluoro-4-methylpiperidin-4-ol,
2-((3S,4R)-1-(4-(8-((2R,3S)-3-(ethylsulfonylmethyl)-2-methylazetidin-1-yl)-5-isopropylisoquinolin-3-ylamino)pyrimidin-2-yl)-3-fluoropiperidin-4-yloxy) ethanol,
(3S,4S)-5,5-difluoro-1-(4-(5-isopropyl-8-((2R,3S)-2-methyl-3-(methylsulfonylmethyl) azetidin-1-yl)isoquinolin-3-ylamino)pyrimidin-2-yl)-4-methoxypiperidin-3-ol,
(3R,4R)-5,5-difluoro-1-(4-(5-isopropyl-8-((2R,3S)-2-methyl-3-(methylsulfonylmethyl) azetidin-1-yl)isoquinolin-3-ylamino)pyrimidin-2-yl)-4-methoxypiperidin-3-ol,
(3S,4S)-1-(4-(8-((2R,3S)-3-(ethylsulfonylmethyl)-2-methylazetidin-1-yl)-5-isopropylisoquinolin-3-ylamino)pyrimidin-2-yl)-4-methoxypiperidin-3-ol,
(3R,4R)-1-(4-(8-((2R,3S)-3-(ethylsulfonylmethyl)-2-methylazetidin-1-yl)-5-isopropylisoquinolin-3-ylamino)pyrimidin-2-yl)-4-methoxypiperidin-3-ol,
a pharmaceutically acceptable salt thereof, or a pharmaceutical composition comprising the same.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib, glumetinib, tepotinib) and MET antibodies (emibetuzumab, telisotuzumab vedotin (ABBV 339)); mitotic kinase inhibitors (CDK4/6 inhibitors such as palbociclib, ribociclib, abemacicilb, GIT38); anti-angiogenic agents e.g., bevacizumab, nintedanib; apoptosis inducers such as Bcl-2 inhibitors e.g. venetoclax, obatoclax, navitoclax, palcitoclax (APG-1252), and Mcl-1 inhibitors e.g., AZD-5991, AMG-176, S-64315; mTOR inhibitors e.g. rapamycin, temsirolimus, everolimus, ridoforolimus; RET inhibitors, like pralsetinib and selpercatinib, and PI3K inhibitors dactolisib (BEZ235), pictilisib (GDC-0941), LY294002, idelalisib (CAL-101); JAK inhibitors (e.g., AZD4205, itacitinib), Aurora A inhibitors (e.g., alisertib); BCR/ABL and/or Src family tyrosine kinase inhibitors (e.g., dasatinib); VEGF inhibitors (e.g., MP0250; ramucirumab); multi-kinase protein inhibitors (e.g., anlotinib, midostaurin); PARP inhibitors (e.g., niraparib); platinum therapies (e.g., cisplatin (CDDP), carboplatin (CBDCA), or nedaplatin (CDGP)); PD-L1 inhibitors (e.g., durvalumab (MEDI 4736)); HER2/neu receptor inhibitors (e.g., trastuzumab); anti-HER2 or anti-HER3 antibody-drug conjugates (e.g., patritumab deruxtecan (U3-1402), trastuzumab emtansine); or immunogene therapy (e.g., oncoprex).

A “subject” is a human in need of treatment.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (A) or (I) being used by following, for example, dosages reported in the literature and recommended in the Physician's Desk Reference (57th Ed . . . 2003).

“Treating” or “treatment” refers to obtaining a desired pharmacological and/or physiological effect. The effect can be therapeutic, which includes achieving, partially or substantially, one or more of the following results; partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition). Mack Publishing Co., Easton. Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed EGFR inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The compounds of the disclosure can be prepared according to the following general synthetic methods.

### Generic Synthetic Schemes

According to a first process, compounds of Formula (I)(A), wherein L¹ is a bond, may be prepared from the compounds of Formulae (II), (III) and (IV), as illustrated by Scheme 1A.

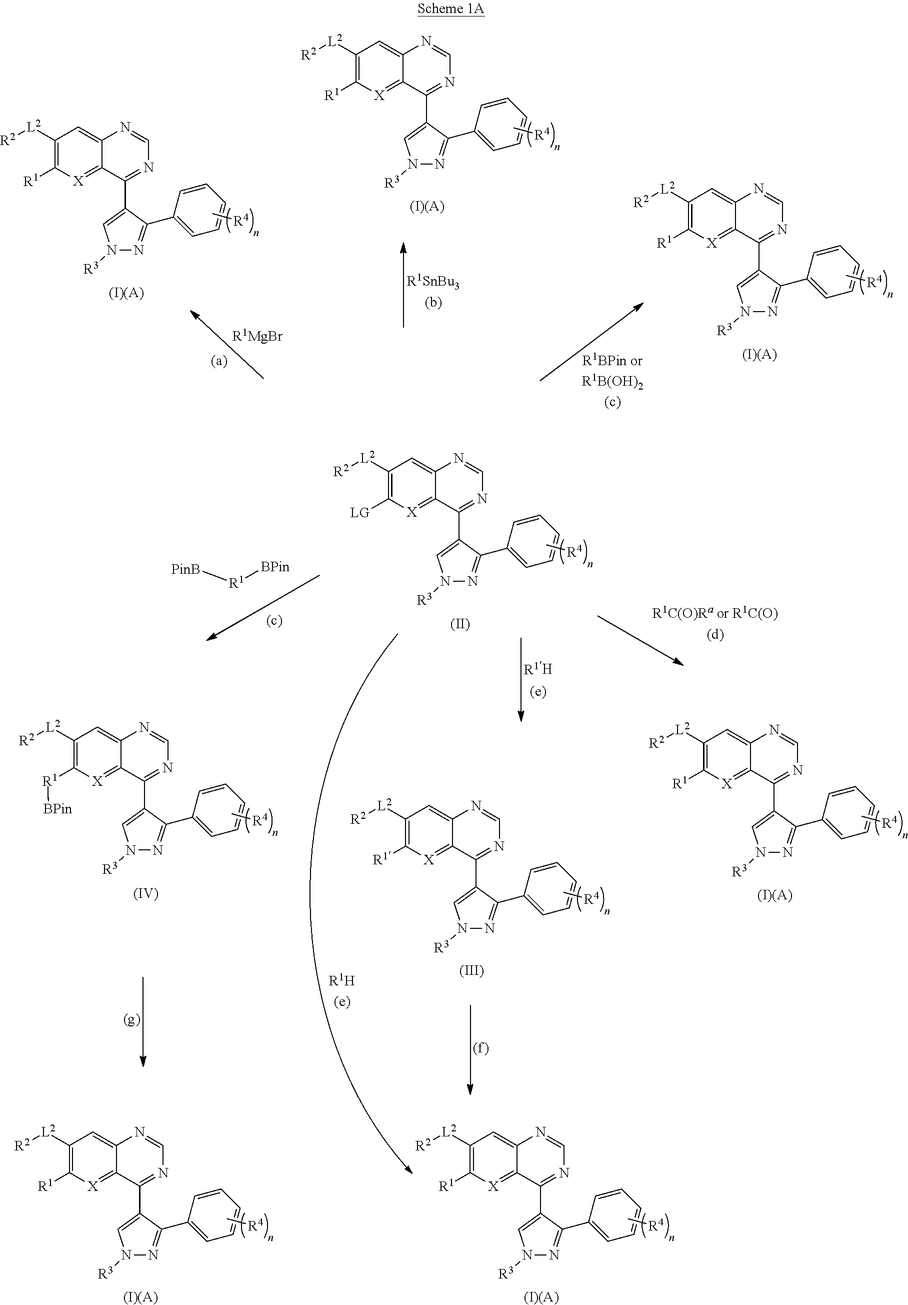

LG is a suitable leaving group, typically halo or triflate and preferably, Cl or Br or triflate The compound of Formula (I)(A) may be prepared from the compound of Formula (II) and R¹MgBr, by process step (a) an Fe catalysed cross-coupling reaction with a Grignard reagent in the presence of NMP as described by Muñoz et. al. Angew. Chem. Int. Ed. 2018, 57, 6496. Preferred conditions comprise, reaction of the compound of Formula (II) with R¹MgBr, Fe (III) acetylacetone, NMP in THF at between 0° C. and rt.

Alternatively, the compound of Formula (I)(A) may be prepared from the compound of Formula (II) and R¹Sn(alkyl)₃, by process step (b) a palladium catalysed cross-coupling reaction with a suitable alkyl or aryl stannane, a Stille Reaction. Typical cross-coupling reaction conditions comprise a palladium catalyst containing suitable phosphine ligands, in the presence of an inorganic base, in a suitable aqueous solvent at between rt and the reflux temperature of the reaction. Preferred conditions comprise reaction of the compound of Formula (II) with R¹SnBu₃, in the presence of a suitable catalyst such as Pd(PPh₃)₂Cl₂, Pd(PPh₃)₄, optionally in the presence of an additive, typically LiCl, in a suitable solvent such as dioxane, at elevated temperature, such as 90-100° C. Alternatively, the compound of Formula (I)(A) may be prepared from the compound of Formula (II) and R¹BPin or R¹B(OH)₂ according to process step (c) a palladium catalysed, cross-coupling reaction, such as a Suzuki reaction. Typical cross-coupling reaction conditions comprise reaction of the compound of Formula (II) with R¹BPin or R¹B(OH)₂, and a palladium catalyst containing suitable phosphine ligands, in the presence of an inorganic base, in a suitable aqueous solvent at between rt and the reflux temperature of the reaction. Preferred conditions comprise, reaction of the compound of Formula (II) and R¹BPin or R¹B(OH)₂, in the presence of Xphos Pd G3, Pd(dppf)Cl₂, Pd₂(dppf)₃ Pd(PPh₃)₄, cataCXium A Pd G3 or PdCl₂(Amphos)₂ and a suitable base such as K₃PO₄, KHCO₃, K₂CO₃ or Cs₂CO₃ in a suitable solvent such as aqueous dioxane, DME or DMSO at between 70° C. and 100° C.

Alternatively, compounds of Formula (I)(A), wherein R¹ is C₁-C₄ alkyl or C₃-C₆cycloalkyl substituted by OH, may be prepared from the compound of Formula (II) and R¹C(O)R^{a} or R¹C(O), by process step (d). Preferred conditions comprise reaction of the compound of Formula (II) and R¹C(O)R^{a} or R¹C(O) in the presence of a strong base such as n-BuLi, in a suitable solvent such as THF at low temperature, such as −78° C.

Compounds of Formula (III), wherein R¹ is an alkyl, cycloalkyl, or heterocyclyl and R^{1′} is its unsaturated pre-cursor, may be prepared from the compounds of Formula (II) and R^{1′}H by process step (c), a palladium catalysed, cross-coupling reaction, such as a Heck reaction. Preferred conditions comprise reaction of the compound of Formula (II) with R^{1′}H in the presence of a suitable palladium catalyst such as Pd(dppf)Cl₂ or Pd(PPh₃)₄, in the presence of a suitable base such as K₂CO₃ or K₃PO₄, in a suitable solvent such as aqueous dioxane, or THF at elevated temperature such as 80° C.

The compound of Formula (I)(A, may be prepared from the compound of Formula (III) by process step (f) a reduction reaction. Preferred conditions comprise reaction of the compound of Formula (III) with a suitable reducing agent such as NaCNBH₃ in the presence of AcOH in a suitable alcoholic solvent such as MeOH at rt.

Alternatively, wherein R¹ is heteroaryl, the compound of Formula (I)(A) may be prepared from the compound of Formula (II) and R¹H by process step (c) as previously described.

Compounds of Formula (IV)) may be prepared from the compound of Formula (II) and R¹(BPin)₂ by process step (c) as previously described above. The compound of Formula (I)(A), wherein R¹ is a cycloalkyl group substituted by OH may be prepared from the compound of Formula (IV) by process step (g) an oxidation reaction. Typical conditions comprise reaction of the compound of Formula (IV) with a suitable oxidising agent such as NaBO₃ in a suitable solvent such as aqueous THF at about rt.

According to a second process, compounds of Formula (I)(A), wherein L¹ is a bond, may be prepared from the compounds of Formulae (II), (V), (VI) and (VII), as illustrated by Scheme 1B.

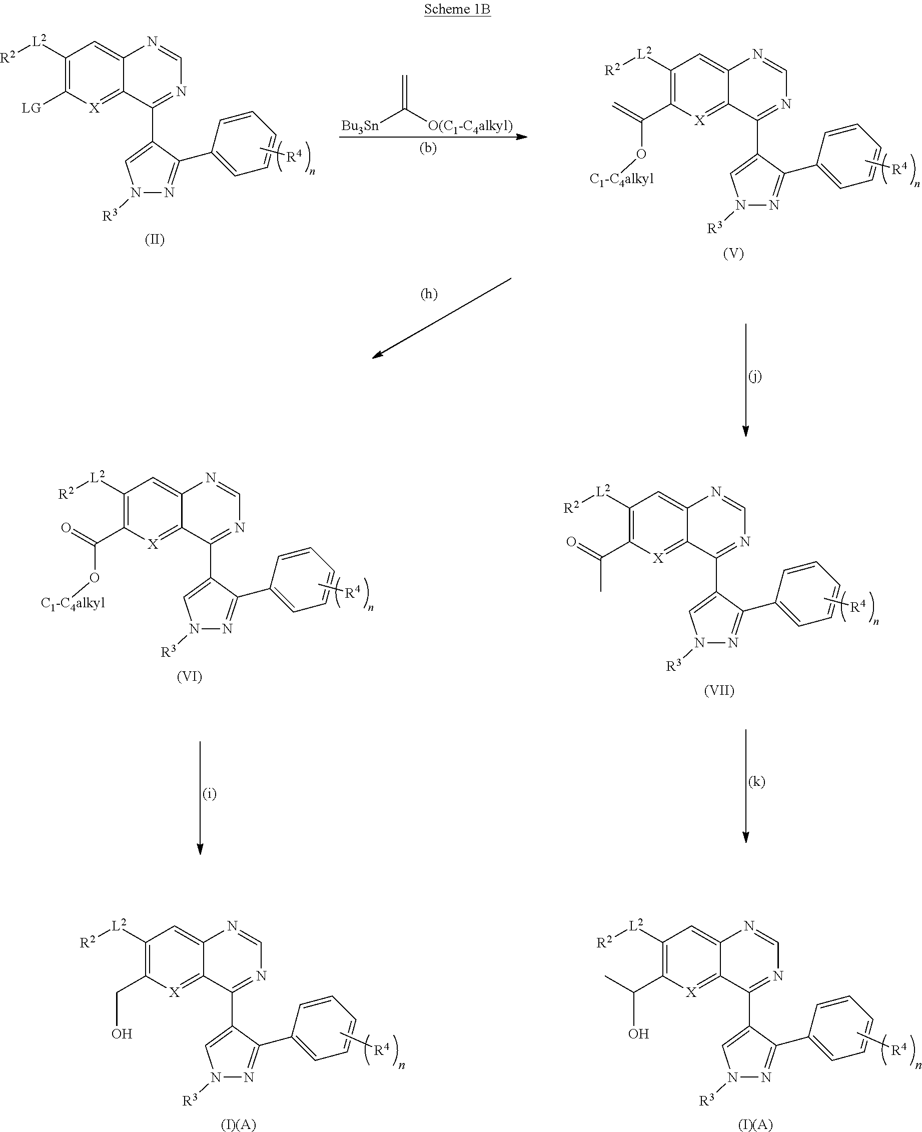

The compound of Formula (V) may be prepared from the compound of Formula (II) and (C₁-C₄alkyl)OC(CH)Sn(alkyl)₃, by process step (b) a palladium catalysed cross-coupling reaction with a suitable alkyl stannane, a Stille Reaction as previously described in Scheme 1A.

The compound of Formula (VI) may be prepared from the compound of Formula (V) by process step (h) an oxidation reaction. Typical conditions comprise reaction of the compound of Formula (V) with KMnO₄, NaIO₄, in a suitable solvent such as aqueous DCM at rt.

The compound of Formula (I)(A) may be prepared from the compound of Formula (VI), according to process step (i) a reduction reaction of an ester. Typical conditions comprise reaction of the compound of Formula (VI) with DIBAL-H in a suitable solvent such as THF at low temperature, such as −30° C.

The compound of Formula (VII) may be prepared from the compound of Formula (V) by process step (j). Typical conditions comprise reaction of the compound of Formula (V) with TFA in DCM at rt.

The compound of Formula (I)(A) may be prepared from the compound of Formula (VII) by process step (k) a reduction reaction of a ketone. Typical conditions comprise reaction of the compound of Formula (VII) with a suitable reducing agent such as NaBH₄ in a suitable solvent such as MeOH.

According to a third process, compounds of Formula (I)(B), wherein L¹ is O, may be prepared from the compound of Formula (VIII), as illustrated by Scheme 1C.

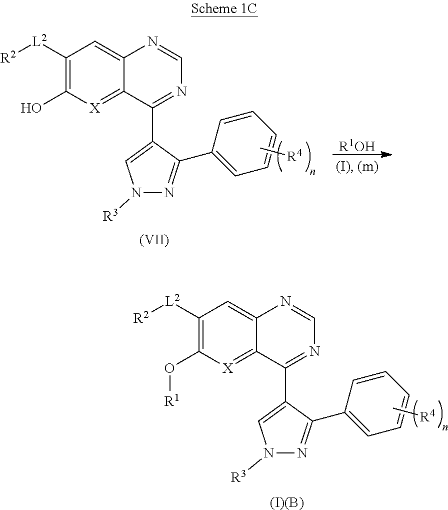

The compound of Formula (I)(B) may be prepared from the compound of Formula (VIII) according to process step (1) a Mitsunobu reaction. Typical conditions comprise reaction of the alcohol of Formula (VIII) with R¹OH in the presence of PPh₃ and a suitable azo dicarboxylate such as DEAD, DIAD or DBAD in a suitable solvent such as THF at between −10° C. and rt.

Alternatively, the compound of Formula (I)(B) may be prepared from the compound of Formula (VIII) and R¹OH by process step (m) an alkylation reaction. Typical conditions comprise reaction of the compound of Formula (VIII) with R¹OH in the presence of an inorganic base such as NaH, KOtBu, KOH or K₂CO₃ in a suitable solvent such as DMF or THF at between rt and elevated temperature such as 80° C.

According to a fourth process, compounds of Formula (I)(B), wherein L¹ is O, may be prepared from the compound of Formula (II) as illustrated by Scheme 1D

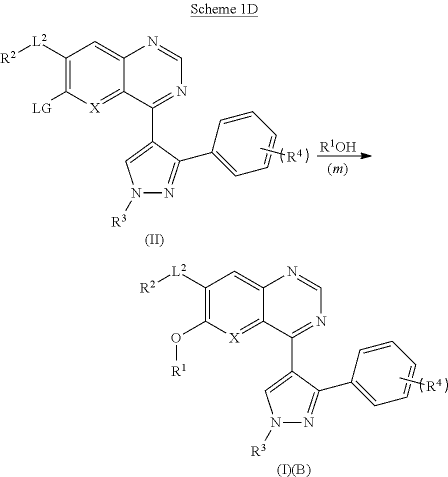

The compound of Formula (I)(B) may be prepared from the compound of Formula (II) and R¹OH by an alkylation reaction as previously described in Scheme 1C above.

According to a fifth process, the compound of Formula (I)(C), wherein L¹ is OC(O) may be prepared from the compounds of Formulae (VIII) and (IX), as illustrated by Scheme 1E

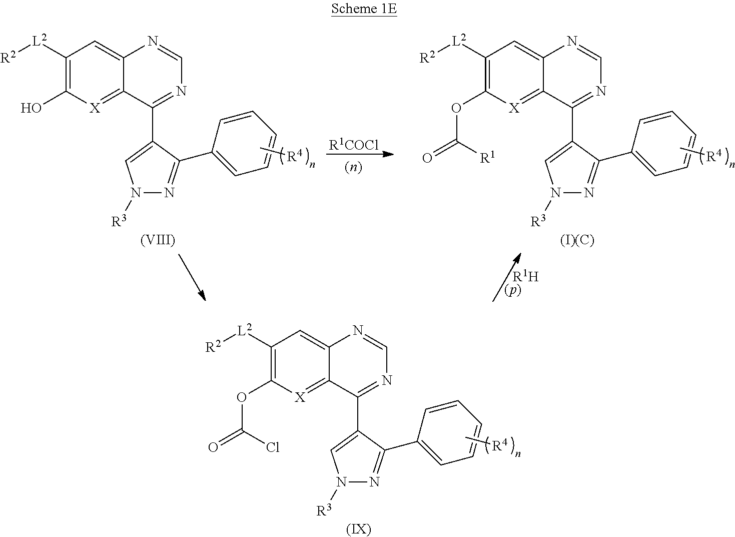

The compound of Formula (IX) may be prepared from the compound of Formula (VIII) by process step (o) a phosgenation reaction. Typical conditions comprise reaction of the compound of Formula (VIII) with phosgene or triphosgene in the presence of a suitable organic base, such as DIPEA in a suitable solvent, such as THF at low temperatures to rt.

The compound of Formula (I)(C) may be prepared from the compound of Formula (IX) and R¹H according to process step (p). Typical conditions comprise reaction of the compound of Formula (IX) with R¹H in the presence of suitable organic base, such as TEA in a suitable organic solvent such as DCM at about rt.

Alternatively, the compound of Formula (I)(C) may be prepared from the compound of Formula (VIII) and R¹COCl, by process step (n). Typical conditions comprise reaction of the compound of Formula (VIII) with R¹COCl in the presence of a suitable inorganic or organic base such as K₂CO₃ or TEA or pyridine in a suitable solvent such as DMF, THF, MeCN or DCM at between rt and elevated temperature such as 80° C.

According to a sixth process, compounds of Formula (I)(D), wherein L¹ is NH, or wherein L¹ is a bond and R¹ is an N-linked heterocycle, may be prepared from the compounds of Formulae (II), as illustrated by Scheme IF

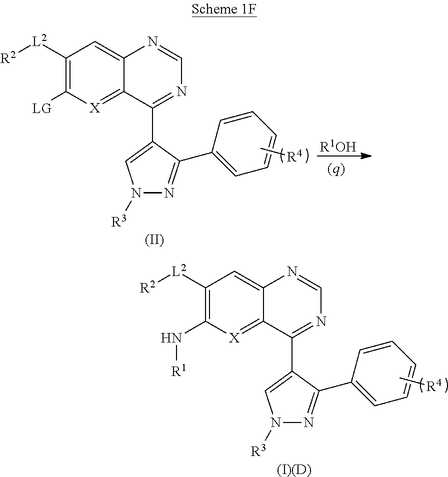

The compound of Formula (I)(D) may be prepared from the compound of Formula (II) and R¹NH₂ according to process step (q) a Buchwald-Hartwig cross-coupling. Typical conditions comprise, reaction of the compound of Formula (II) with R¹NH₂ in the presence of a suitable inorganic base, a suitable catalyst in a suitable solvent at elevated temperature. Preferred conditions comprise, reaction of the compounds of Formula (II) and R¹NH₂ in the presence of, BrettPhos Pd G3, Rockphos Pd, Xantphos Pd G3, RuPhos Pd, E-Phos Pd G4, PEPPSI Pd-Ipent-O-Picoline or BINAP Pd G2 or Xphos or Xphos Pd G3 in combination with Pd₂(dba)₃ in the presence of a suitable base such as Cs₂CO₃, K₂CO₃, K₃PO₄, KOAc or NaOtBu in a suitable solvent such as toluene, dioxane or MeCN, at between 80° C. and 120° C.

Alternatively, the compound of Formula (I)(D) may be prepared from the compound of Formula (II) and R¹NH₂ according to process step (z), an amination reaction. Preferred conditions comprise reaction of the compound of Formula (II) and R¹NH₂, optionally in the presence of an organic or inorganic base, optionally in a suitable solvent such as DMSO at elevated temperature, such as 100° C.

According to a seventh process, compounds of Formula (I)(E), wherein L¹ is NHC(O), may be prepared from the compound of Formula (X), as illustrated by Scheme IG.

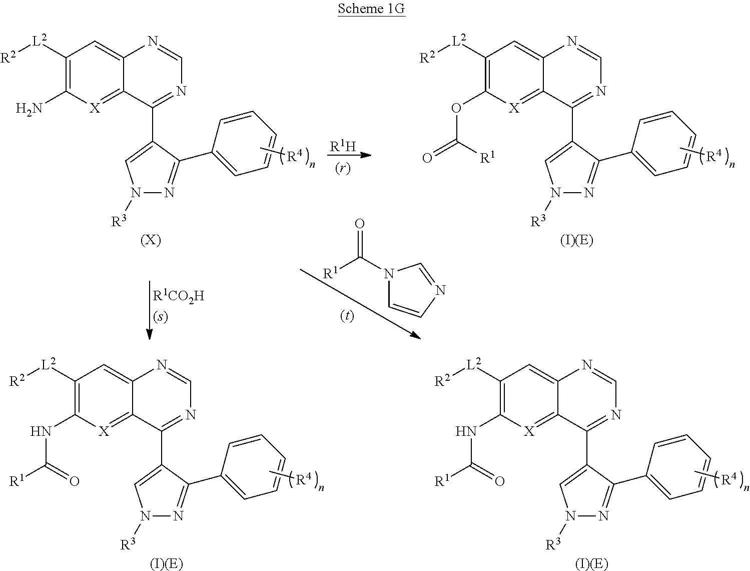

The compound of Formula (I)(E) may be prepared from the compound of Formula (X) and R¹H according to process step (r). Typical conditions comprise reaction of the compound of Formula (XX) with a suitable “carbonylating agent”, such as 4-nitrophenyl chloroformate, 2,4,6-trichlorobenzoyl chloride or CDI, in the presence of a suitable organic base such as pyridine or TEA, optionally in a suitable solvent such as DCM at between −78° C. and 60° C., followed by reaction with R¹H optionally in the presence of an organic base such as TEA in a suitable solvent such as DCM at between rt and about 80° C.

Alternatively, the compound of Formula (I)(E) may be prepared from the compound of Formula (X) and R¹C(O)Cl according to process step (n) as previously described in Scheme 1E above. Alternatively, the compound of Formula (I)(E) may be prepared from the compound of Formula (X) and R¹CO₂H according to process step(s), an amide bond forming reaction, in the presence of a suitable coupling agent and organic base, optionally in a suitable polar aprotic solvent. Preferred conditions, comprise the reaction of the amine of Formula (X), with R¹CO₂H in the presence of coupling agent preferably, T3P®, CDI or HATU in combination with DMAP, in the presence of a suitable organic base such as TEA, DIPEA or pyridine, optionally in a suitable solvent, such as DMF, dioxane or THF at between rt and the reflux temperature of the reaction.

Alternatively, the compound of Formula (I)(E) may be prepared from the compound of Formula (X) and R¹C(O) imidazole according to process step (t). Preferred conditions comprise, reaction of the compound of Formula (X) with R¹C(O) imidazole, in the presence of a strong base such as NaH in a suitable solvent such as THF at low temperature, typically 0° C.

According to an eighth process, compounds of Formula (I)(E), may be prepared from the compound of Formula (II), as illustrated by Scheme 1H.

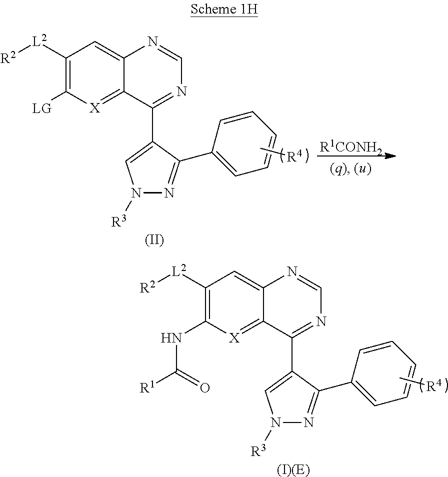

The compound of Formula (I)(E) may be prepared from the compound of Formula (II) and R¹CONH₂ according to process step (q) as previously described in Scheme IF.

Alternatively, the compound of Formula (I)(E) may be prepared from the compound of Formula (II) and R¹CONH₂ according to process step (u), an Ullmann-type, copper mediated coupling reaction. Preferred conditions comprise, reaction of the compound of Formula (II) with R¹CONH₂ in the presence of CuI, a suitable ligand such as N1,N2-dimethylethane-1,2-diamine or L-proline, a suitable inorganic base such as K₂CO₃ or K₃PO₄, in dioxane or DMSO at between 9° and 120° C. According to a ninth process, compounds of Formula (I)(F), wherein L¹ is NHC(O)O, may be prepared from the compounds of Formulae (X), as illustrated by Scheme II.

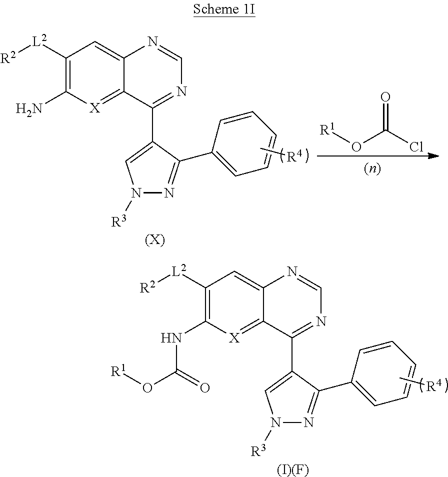

The compound of Formula (I)(F) may be prepared from the compound of Formula (X) and R¹OC(O)Cl according to process step (n) as previously described in Scheme 1E above. According to a tenth process, compounds of Formula (I)(G), wherein L² is O, may be prepared from the compound of Formula (XI), as illustrated by Scheme 2A.

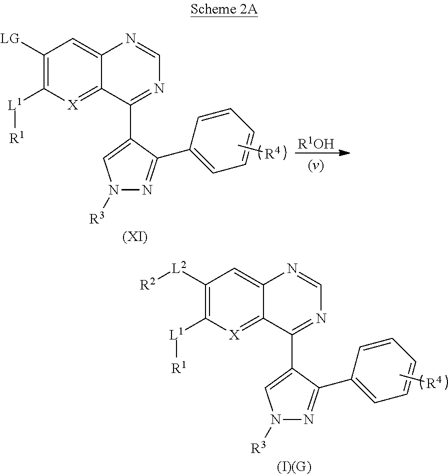

LG is as previously defined.

The compound of Formula (I)(G) may be prepared from the compound of Formula (XI) and R²OH according to process step (v) a Buchwald-Hartwig cross-coupling reaction. Typical conditions comprise, reaction of the compound of Formula (XI) with R²OH in the presence of a suitable inorganic base, a suitable catalyst in a suitable solvent at elevated temperature. Preferred conditions comprise, reaction of the compounds of Formula (XI) and R²OH in the presence of, BrettPhos Pd G3, Rockphos Pd, Xantphos Pd G3, or t-BuXphos and Pd₂(dba)₃ with a suitable base such as Cs₂CO₃ in a suitable solvent such as toluene, DMSO or aqueous dioxane at about 95° C.

According to an eleventh process, compounds of Formula (I)(G), wherein L¹ is O, may be prepared from the compound of Formula (XII), as illustrated by Scheme 2B.

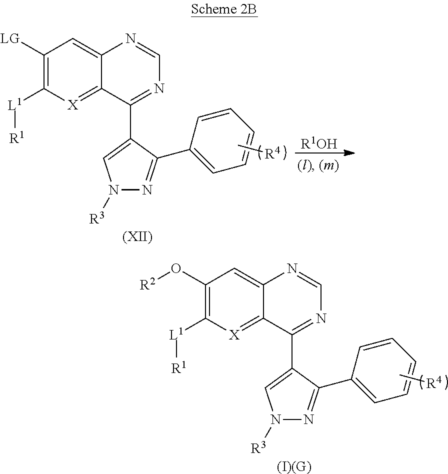

The compound of Formula (I)(G) may be prepared from the compound of Formula (XII) and R²OH according to process step (1) a Mitsunobu reaction, as previously described in Scheme 1C. Alternatively, the compound of Formula (I)(G) may be prepared from the compound of Formula (XII) and R²OH by process step (m) an alkylation reaction, as previously described in Scheme 1C above.

According to a twelfth process, compounds of Formula (I)(H), wherein L¹ is a bond, may be prepared from the compound of Formula (XI), as illustrated by Scheme 2C.

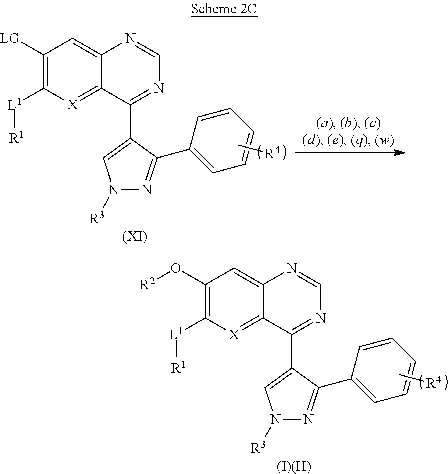

The compound of Formula (I)(H) may be prepared from the compound of Formula (XI) and R²MgBr by process step (a) as previously described in Scheme 1A above.

Alternatively, the compound of Formula (I)(H) may be prepared from the compound of Formula (XI) and R²SnBu₃ by process step (b) as previously described in Scheme 1A above.

Alternatively, the compound of Formula (I)(H) may be prepared from the compound of Formula (XI) and R²BPin by process step (c) as previously described in Scheme 1A above.

Alternatively, The compound of Formula (I)(H) may be prepared from the compound of Formula (XI) and R²C(O)R^{a} by process step (d) as previously described in Scheme 1A above.

Alternatively, The compound of Formula (I)(H) may be prepared from the compound of Formula (XI) and R²H by process step (e) and (f) as previously described in Scheme 1A above.

Alternatively, wherein R² is a C₂-C₄alkynl group, the compound of Formula (I)(H) may be prepared from the compound of Formula (XI) and R²H according to process step (w) a palladium catalysed cross coupling reaction, Sonagashira type reaction. Typical conditions comprise reaction of the compound of Formula (XI) with R²H in the presence of a suitable Cu (I) salt such as CuI, a suitable palladium catalyst such as Pd(PPh₃)₄, in the presence of an organic base such as TEA in DMF at elevated temperature, such as 100° C.

Alternatively, wherein R² is an N-linked heterocycle, the compound of Formula (I)(H) may be prepared from the compound of Formula (XI) and R²H, according to process step (q) a Buchwald reaction as previously described in Scheme IF above.

According to a thirteenth process, compounds of Formula (I), may be prepared from the compounds of Formulae (XIII) and (XIV), as illustrated by Scheme 3.

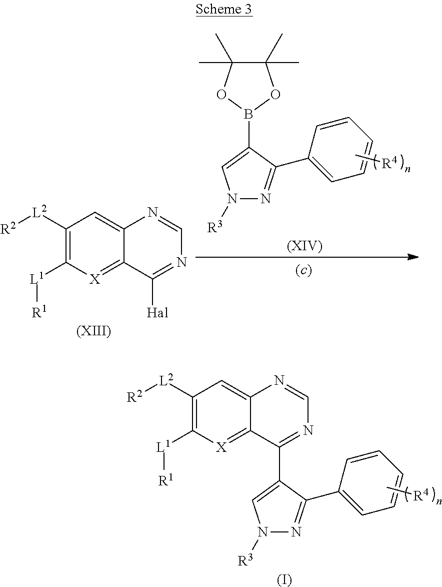

Hal is a halogen, preferably Cl or Br.

The compound of Formula (I) may be prepared from the compounds of Formulae (XIII) and (XIV) according to process step (c) a Suzuki type cross-coupling reaction, as previously described in Scheme 1A above.

According to a fourteenth process, compounds of Formula (II), may be prepared from the compounds of Formulae (XV), (XVI) and (XIV), as illustrated by Scheme 4.

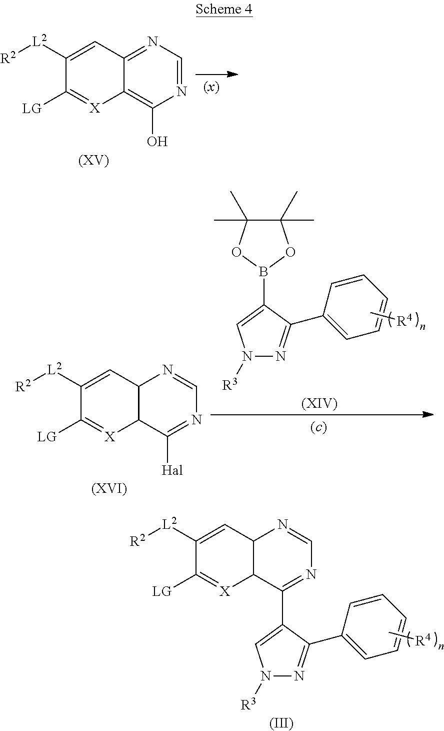

The compound of Formula (XVI) may be prepared from the compound of Formula (XV) by process step (x) a halogenation reaction reaction, with a suitable halogenating agent. Typical conditions comprise reaction of the compound of Formula (XV) with a suitable halogenating agent such as POCl₃ or CCl₄ or POBr₃ in the presence of PPh₃, optionally in the presence of an organic base such as DIPEA or N,N-diethylaniline, optionally in a suitable solvent such as DCE or MeCN at elevated temperature such as 70 to 100° C.

The compound of Formula (II) may be prepared from the compounds of Formulae (XVI) and (XIV) by process step (c) a Suzuki type cross-coupling reaction, as previously described in Scheme 1A above.

According to a fifteenth process, compounds of Formula (VIII), may be prepared from the compounds of Formulae (II), (XIV) and (XVII), as illustrated by Scheme 5.

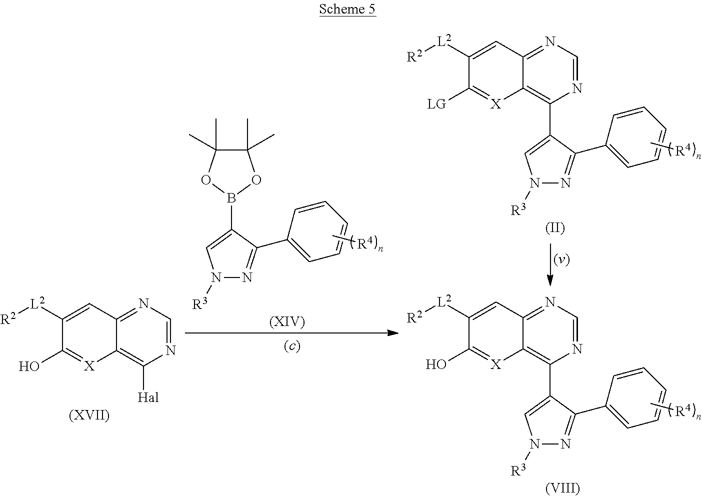

The compound of Formula (VIII) may be prepared from the compound of Formula (II) by process step (v) a Buchwald type cross coupling reaction as previously described in Scheme 2A above.

The compound of Formula (VIII) may be prepared from the compounds of Formulae (XVII) and (XIV) by process step (c) a Suzuki type cross-coupling reaction, as previously described in Scheme 1A above.

According to a sixteenth process, compounds of Formula (XI), may be prepared from the compounds of Formulae (XVIII) and (XIX), as illustrated by Scheme 6.

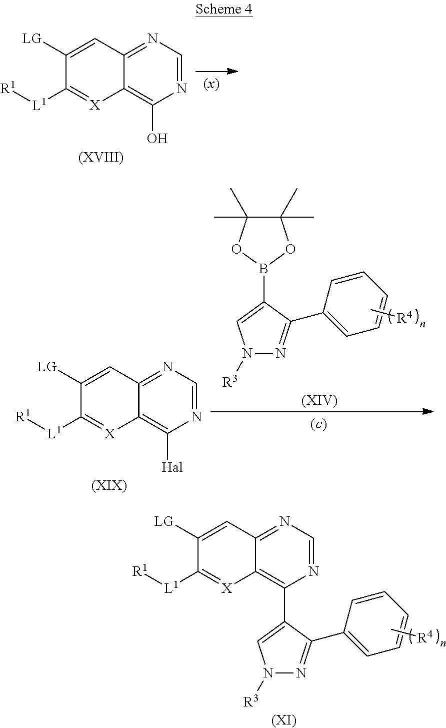

The compound of Formula (XIX) may be prepared from the compound of Formula (XVIII) by process step (x) a halogenation reaction as described previously in Scheme 4.

The compound of Formula (XI) may be prepared from the compounds of Formulae (XIX) and (XIV) according to process step (c) as previously described in Scheme 1A.

According to an eighteenth process, compounds of Formula (XII), may be prepared from the compounds of Formulae (XX) and (XIV), as illustrated by Scheme 7.

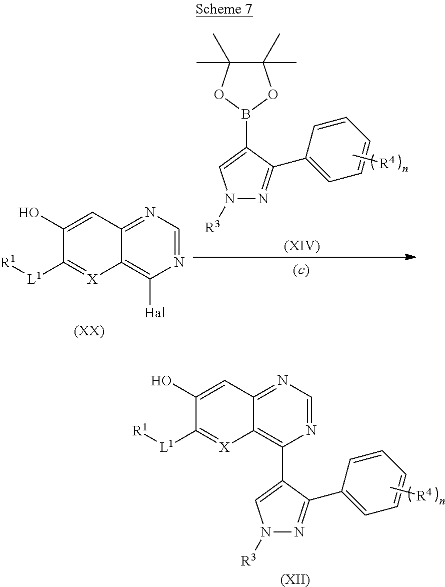

The compound of Formula (XII) may be prepared from the compounds of Formulae (XX) and (XIV) according to process step (c) as previously described in Scheme 1A.

According to a nineteenth process, compounds of Formula (X), may be prepared from the compounds of Formulae (XIV), (XXI) and (XXII), as illustrated by Scheme 8.

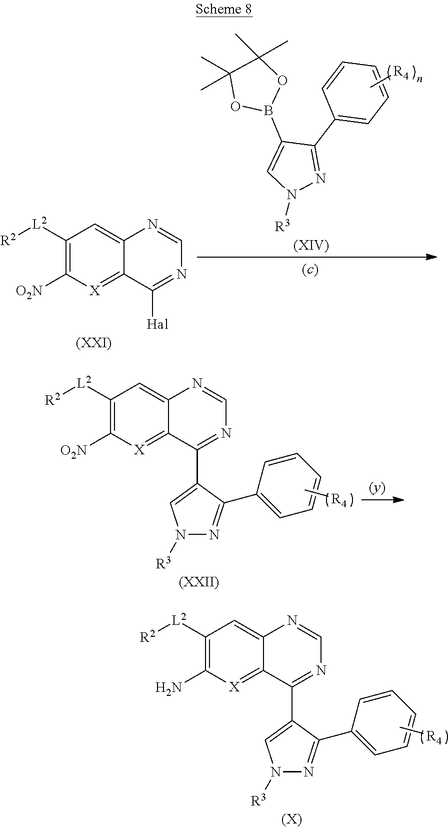

The compound of Formula (XXII) may be prepared from the compounds of Formulae (XXI) and (XIV) by process step (c) a Suzuki cross coupling reaction as previously described in Scheme 1A.

The compound of Formula (X) may be prepared from the compound of Formula (XXII) by process step (y) a reduction of a nitro group. Typical conditions comprise reaction of the compound of Formula (XXII) with iron in the presence of a mild acid, in a suitable alcoholic solvent such as aqueous EtOH at elevated temperature e.g., 80° C.

According to a twentieth process, compounds of Formula (XIII), may be prepared from the compounds of Formulae (XVII) and (XXIII), as illustrated by Scheme 9.

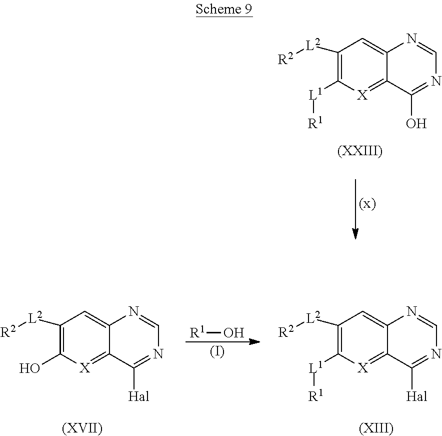

The compound of Formula (XIII) may be prepared from the compound of Formula (XVII) and R¹OH according to process step (1), a Mitsunobu reaction as previously described in Scheme 1C.

Alternatively, the compound of Formula (XIII) may be prepared from the compound of Formula (XXIII) according to process step (x), a halogenation reaction as previously described in Scheme 4.

The compounds of Formulae (XIV), (XV), (XVII), XVIII), (XX), (XXI) and (XXIII) are either commercially available or may be prepared by analogy to methods known in the literature, or the methods described in the Experimental section below.

Compounds of Formula (I), (II), (VIII), (X), (XI), (XII), (XIII), (XIV), (XV), (XVI), (XVII), (XVIII), (XIX), (XX), (XXI) and (XXII) may be converted to alternative compounds of Formula (I), (II), (VIII), (X), (XI), (XII), (XIII), (XIV), (XV), (XVI), (XVII), (XVIII), (XIX), (XX), (XXI) and (XXII) by standard chemical transformations, known to those skilled in the art. Examples of these transformations include, but are not limited to: reductive amination of an N atom, alkylation or acetylation of a heteroatom, such as N or O, a Chan-Lam coupling reaction of an N—H containing compound or reduction of an ester to an alcohol.

It will be appreciated by those skilled in the art that it may be necessary to utilize a suitable protecting group strategy for the preparation of compounds of Formula (I). Typical protecting groups may comprise, carbamate and preferably Boc for the protection of amines, a TBS or benzyl group for the protection of a primary alcohol, a benzyl group, methyl or TBDMS for the protection of a phenolic OH, a THP or group for the protection of a pyrazole N atom.

It will be further appreciated that it may be necessary or desirable to carry out the transformations in a different order from that described in the schemes, or to modify one or more of the transformations, to provide the desired compound of the invention.

The following examples are intended to be illustrative and are not intended to be limiting in any way to the scope of the disclosure.

### EXEMPLIFICATION

Preparation of Exemplary Compounds

Definitions

| | |
|---|---|
| ° C. | degrees Celsius |
| CC | Silica Column Chromatography |
| CAN, MeCN | acetonitrile |
| AcOH | acetic Acid |
| DCM | dichloromethane |
| DIEA | diisopropylethylamine |
| DMF | dimethyl formamide |
| DMF—DMA | N,N-Dimethylformamide dimethyl acetal |
| DMSO | dimethylsulfoxide |
| EA | ethyl acetate |
| EDCI | N-(3-Dimethylaminopropyl)-N′-ethylcarbodiimide |
| EtOAc | Ethyl acetate |
| H, h, hr(s) | hour(s) |
| HOBt | 1-Hydroxybenzotriazole |
| HPLC | high performance liquid chromatography |
| IC₅₀ | inhibitory concentration 50% |
| i-PrOH | isopropyl alcohol |
| IPA | isopropyl alcohol |
| min | minutes |
| MTBE | methyl tert-butyl ether |
| MeOH | methanol |
| Pd₂(dba)₃ | Tris(dibenzylideneacetone)dipalladium(0) |
| Pd(dppf)Cl₂•DCM | [1,1′-Bis(diphenylphosphino)ferrocene]dichloro- |
| | palladium(II), complex with dichloromethane |
| PE | petroleum ether |
| rt | room temperature |
| TEA | triethylamine |
| TFA | trifluoracetic Acid |
| THF | tetrahydrofuran |
| RT | retention time |
| Prep HPLC | preparative high-performance liquid chromatography |
| Prep-TLC | preparative thin layer chromatography |
| TLC | thin layer chromatography |
| MsCl | methanesulfonyl chloride |
| Bpin | boronic acid pinacol ester |
| BINAP | (2,2′-bis(diphenylphosphino)-1,1′-binaphthyl) |
| Pd(dppf)Cl₂ | [1,1′-Bis(diphenylphosphino)ferrocene]dichloro- |
| | palladium |
| XPhos | 2-Dicyclohexylphosphino-2′,4′,6′- |
| | triisopropylbiphenyl |

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety. Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization:

LC-MS: liquid chromatography-mass spectrometry (LC-MS) data (sample analyzed for purity and identity) were obtained with:
an Agilent model-1260 LC system using an Agilent model 6120 mass spectrometer utilizing ES-API ionization fitted with an Agilent Poroshel 120 (EC-C18, 2.7 μm particle size, 3.0×50 mm dimensions) reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 4 minutes was utilized. The flow rate was constant at 1 mL/min; or
Shimadzu LCMS system using a Shimadzu LCMS mass spectrometer utilizing ESI ionization fitted with an Agilent (Poroshel HPH-C18 2.7 μm particle size, 3.0×50 mm dimensions) reverse-phase column at 22.4 degrees Celsius. The mobile phase consisted of a mixture of solvent 5 mM NH₄HCO₃ (or 0.05% TFA) in water and acetonitrile. A constant gradient from 90% aqueous/10% organic to 5% aqueous/95% organic mobile phase over the course of 2 minutes was utilized. The flow rate was constant at 1.5 mL/min.

Prep LC-MS: Preparative HPLC was performed on a Shimadzu Discovery VP® Preparative system fitted with a Luna 5u C18(2) 100A, AXIA packed, 250×21.2 mm reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 25 minutes was utilized. The flow rate was constant at 20 mL/min.

Manufacturer: Yantai Xinnuo Chemicals Co, particle size: 10-40 um, PH=6.2-7, Thickness: 1 mm, Binder: CMC, Specifications: 200*200 mm

Silica gel chromatography: Silica gel chromatography was performed on either a Teledyne Isco CombiFlash® Rf unit or a Biotage® Isolera Four unit or a Biotage® Isolera Prime unit.

Proton NMR: ¹H NMR spectra were obtained with
a Varian 400 MHz Unity Inova 400 MHz NMR instrument (acquisition time=3.5 seconds with a 1 second delay: 16 to 64 scans). Where characterized, all protons were reported in DMSO-d6 solvent as parts-per million (ppm) with respect to residual DMSO (2.50 ppm); or
a Avance 400 MHz Unity Inova 400 MHz NM instrument (acquisition time=3.99 seconds with a 1 second delay: 4 to 64 scans) or a Avance 300 MHz Unity Inova 300 MHz NMR instrument (acquisition time=5.45 seconds with a 1 second delay; 4 to 64 scans).

Prep LC-MS: Preparative HPLC was performed on a Waters Preparative system fitted with Column: XBridge Shield RP18 OBD Column, 30*150 mm, 5 um: The mobile phase consisted of a solvent mixture of aqueous: (Water (10 mmol/L NH₄HCO₃+0.05% NH₃·H₂O)) and organic (acetonitrile). A constant gradient from 95% aqueous/5% organic to 5% aqueous/95% organic mobile phase was utilized. The flow rate was constant and typically 60 mL/min.

Reactions carried out in a microwave were done so in a Biotage Initiator microwave unit. One of ordinary skill in the art will recognize that modifications of the gradient, column length, and flow rate are possible and that some conditions may be more suitable for compound characterization than others, depending on the chemical species being analyzed.

### PREPARATION OF INTERMEDIATES

### Preparation 1: 1-ethyl-3-phenyl-1H-pyrazole

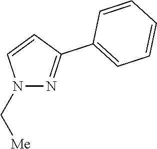 [SMILES: CCn1ccc(-c2ccccc2)n1]

Cs₂CO₃ (441 g, 1.35 mol) was added to a solution of 3-phenyl-1H-pyrazole (65 g, 451 mmol) in DMF (250 mL) and the mixture warmed to 45-60° C. Ethyl iodide (141 g, 902 mL) was added drop wise and the reaction stirred overnight at 45-60° C. The cooled mixture was diluted with water (2.5 L) and extracted with EtOAc (700 mL×3). The combined organic extracts were washed with brine (1 L×3), dried over Na₂SO₄ filtered and the filtrate evaporated under reduced pressure to afford the title compound, 80.8 g. LCMS m/z=173.1 [M+H]⁺.

### Preparation 2: 1-(2,2-difluoroethyl)-3-phenyl-1H-pyrazole

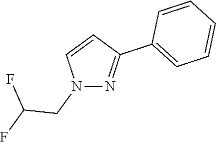 [SMILES: FC(F)Cn1ccc(-c2ccccc2)n1]

To a solution of 3-phenyl-1H-pyrazole (84.0 g, 582 mmol) and Cs₂CO₃ (569 g, 1.75 mol) in DMF (900 mL) was added 1,1-difluoro-2-iodoethane (223 g, 1.17 mol) dropwise at 45° C. and the mixture was stirred at 45° C. for 12 h. The reaction mixture was diluted with H₂O (1.50 L) and extracted with EtOAc (1.50 L×3). The combined organic layers were washed with brine (2.00 L×2), dried over (Na₂SO₄), filtered and concentrated under reduced pressure. The residue was purified by column chromatography (SiO₂, PE:EtOAc=20:1 to 3:1) to give the title compound (137 g, crude) as a light yellow oil. LCMS m/z=209.2 [M+H]⁺.

### Preparation 3: 1-ethyl-3-(4-fluorophenyl)-1H-pyrazole

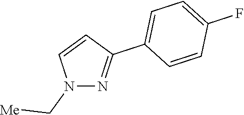 [SMILES: CCn1ccc(-c2ccc(F)cc2)n1]

NaH (295 mg, 12.3 mmol) was added to 3-(4-fluorophenyl)-1H-pyrazole (2 g, 12.3 mmol) in DMF (20 mL) and the solution stirred for 30 min. Ethyl iodide (1.91 g, 12.3 mmol) was added and the resulting solution was stirred for 1 h at rt. The reaction was quenched with water (5 mL) and the resulting solution was extracted with DCM (3×10 mL). The combined organic extracts were evaporated under reduced pressure to give the title compound as a light yellow solid, 1.5 g, 64.3% yield. LCMS m/z=191 [M+H]⁺.

### Preparation 4: 1-ethyl-3-(2-fluorophenyl)-1H-pyrazole

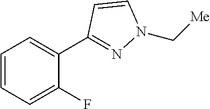 [SMILES: CCn1ccc(-c2ccccc2F)n1]

NaH (590 mg, 24.6 mmol) was added slowly to 3-(2-fluorophenyl)-1H-pyrazole (2 g, 12.3 mmol) in DMF (10 mL) at 0° C. and the mixture stirred for 30 min before iodoethane (3.83 g, 24.6 mmol) was added to the mixture at 0° C. and the mixture stirred at 60° C. for 16 h. The solution was diluted with water (500 mL) and extracted with EtOAc (3×200 mL). The combined organics were dried (Na₂SO₄) and evaporated to dryness in vacuo. The residue was purified by column chromatography (10:1 PE/EtOAc) to afford the title compound as a yellow oil (2 g, 85%). LCMS: m/z=191 [M+H]⁺.

### Preparation 5: 1-(oxetan-3-yl)-3-phenyl-1H-pyrazole

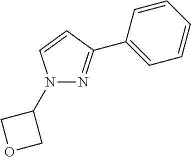 [SMILES: c1ccc(-c2ccn(C3COC3)n2)cc1]

3-Iodooxetane (794 μL, 9.02 mmol) was added to a mixture of 3-phenyl-1H-pyrazole (1.0 g, 6.94 mmol) and NaOtBu (727 mg, 7.56 mmol) in DMF (23.1 mL) and the reaction was stirred at rt for 24 h. Additional NaOtBu (242 mg) and 3-iodooxetane (0.26 mL) were added and the reaction stirred for a further 24 h. The reaction was diluted with water, the layers separated and the aqueous phase was extracted with EtOAc (2×10 mL). The combined organic layers were washed with water (3×5 mL), dried over anhydrous Na₂SO₄, filtered and the solvent removed under reduced pressure. The crude product was purified by ISCO CombiFlash (0-30% EtOAc in hexanes) to give the title compound, as a light-yellow oil, 870 mg, 62.6% yield. ¹H NMR (500 MHz, CDCl₃) δ 7.84 (d, 2H), 7.61 (d, 1H), 7.41 (td, 2H), 7.35-7.28 (m, 1H), 6.63 (d, 1H), 5.57-5.44 (m, 1H), 5.14 (t, 2H), 5.10-5.03 (m, 2H).

### Preparation 6: 3-(2-fluorophenyl)-1-methyl-1H-pyrazole

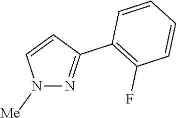 [SMILES: Cn1ccc(-c2ccccc2F)n1]

To a vial containing 1-methyl-3-(4,4,5,5-tetramethyl-1,3,2-dioxaborolan-2-yl)-1H-pyrazole (1.5 g, 7.21 mmol) and PdCl₂(dppf):DCM (589 mg, 0.721 mmol) in DME (10.30 mL) under N₂ was added Na₂CO₃ (2.29 g, 21.63 mmol) in water (10.30 mL) followed by 1-bromo-2-fluorobenzene (1.183 mL, 10.81 mmol) and the reaction was stirred at 90° C. for 1 h. The reaction mixture was diluted with water and EtOAc, the organic layer was separated, washed with saturated brine, dried over anhydrous Na₂SO₄, and concentrated in vacuo. The crude product was purified by ISCO Combiflash (0-60% EtOAc in Hexanes) to give the title compound as an orange oil, 637 mg, 50% yield.

### Preparation 7: 3-(2,6-difluorophenyl)-1-methyl-1H-pyrazole

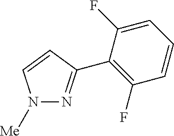 [SMILES: Cn1ccc(-c2c(F)cccc2F)n1]

The title compound was obtained as a yellow oil, 1.05 g, 56% yield, from 2-bromo-1,3-difluorobenzene and 1-methyl-3-(4,4,5,5-tetramethyl-1,3,2-dioxaborolan-2-yl)-1H-pyrazole, following the procedure described in Preparation 6.

### Preparation 8: 3-(4-fluorophenyl)-1-methyl-1H-pyrazole

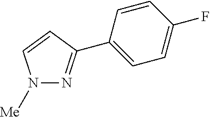 [SMILES: Cn1ccc(-c2ccc(F)cc2)n1]

A mixture of Pd(dppf)Cl₂ (465 mg, 0.571 mmol), K₂CO₃ (1.18 g, 8.56 mmol), 1-bromo-4-fluorobenzene (1 g, 5.71 mmol) and 1-methyl-3-(4,4,5,5-tetramethyl-1,3,2-dioxaborolan-2-yl)-1H-pyrazole (1.78 g, 8.56 mmol) in H₂O (6 mL) and dioxane (24 mL) was stirred at 80° C. for 2 h under N₂. The reaction mixture was diluted with DCM (100 mL) and washed with H₂O (2×50 mL), dried (Na₂SO₄) and evaporated to dryness. The residue was purified by column chromatography (20:1 DCM/MeOH) to afford the title compound as an off-white solid (800 mg, 80%). LCMS: m/z=177 [M+H]⁺.

### Preparation 9: 3-(2,4-difluorophenyl)-1-methyl-1H-pyrazole

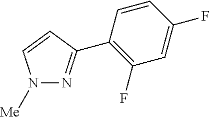 [SMILES: Cn1ccc(-c2ccc(F)cc2F)n1]

The title compound was obtained as a yellow oil, 990 mg, from 1-methyl-3-(4,4,5,5-tetramethyl-1,3,2-dioxaborolan-2-yl)-1H-pyrazole and 1-bromo-2,4-difluorobenzene following the procedure described in Preparation 8. LCMS m/z=195 [M+H]⁺.

### Preparation 10: 1-methyl-3-(3-((tetrahydro-2H-pyran-2-yl)oxy)phenyl)-1H-pyrazole

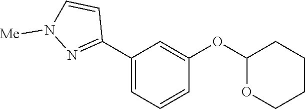 [SMILES: Cn1ccc(-c2cccc(OC3CCCCO3)c2)n1]

A mixture of Pd(dppf)Cl₂ (1.39 g, 1.91 mmol), K₂CO₃ (3.96 g, 28.7 mmol), 1-methyl-3-(4,4,5,5-tetramethyl-1,3,2-dioxaborolan-2-yl)-1H-pyrazole (4 g, 19.2 mmol) and 2-(3-bromophenoxy) tetrahydro-2H-pyran (7.37 g, 28.7 mmol) in dioxane/H₂O (120 mL/30 mL) was stirred at 80° C. for 2 h under N₂. The reaction mixture was extracted with EtOAc (3×150 mL) and the combined organics dried (Na₂SO₄) and evaporated to dryness in vacuo. The residue was purified by silica gel chromatography (2:1 PE/EtOAc) to afford the title compound as a white solid (3.5 g, 70%). LCMS: m/z=259 [M+H]⁺.

### Preparation 11: 3-(2,6-difluorophenyl)-1-methyl-1H-pyrazole

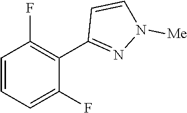 [SMILES: Cn1ccc(-c2c(F)cccc2F)n1]

A mixture of 3-bromo-1-methyl-1H-pyrazole (5 g, 31.0 mmol), (2,6-difluorophenyl) boronic acid (7.34 g, 46.5 mmol), Pd(dtbpf)Cl₂ (2.01 g, 3.10 mmol) and K₃PO₄ (13.1 g, 62.0 mmol) in dioxane (60 mL) and water (10 mL) was stirred at 100° C. for 16 h. The reaction mixture was extracted with EtOAc and the combined organics evaporated to dryness. The residue was purified by column chromatography (20% EtOAc/PE) to give the title compound as a white solid (3 g, 50%). LCMS: m/z=195 [M+H]⁺.

### Preparation 12: 4-bromo-1-ethyl-3-phenyl-1H-pyrazole

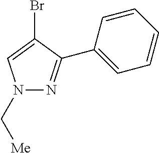 [SMILES: CCn1cc(Br)c(-c2ccccc2)n1]

NBS (87.2 g, 490 mmol) was added to a solution of 1-ethyl-3-phenyl-1H-pyrazole (Preparation 1, 80.8 g, 466 mmol) in MeCN (850 mL) and the reaction stirred at rt for 1 h. The mixture was concentrated in vacuo, the residue dissolved in EtOAc (100 mL) and washed with aq. NaHCO₃ solution (700 mL). The aqueous solution was extracted with EtOAc (200 mL×3), the combined organic phases were washed with brine (250 mL×5), dried over Na₂SO₄, filtered and concentrated in vacuo. The residue was purified by column chromatography on silica gel (PE/EtOAc 20/1 to 10/1) to give a brown oil, 115 g. This was further purified by SFC column: DAICEL CHIRALCEL OJ (250 mm*50 mm, 10 μm), mobile phase: IPA (0.05% DEA) to give the title compound as a yellow oil, 40 g, 35% yield. ¹H NMR (400 MHZ, CDCl₃) δ: 7.90 (q, 2H), 7.49 (s, 1H), 7.46-7.44 (m, 2H), 7.42-7.37 (m, 1H), 4.22-4.17 (m, 2H), 1.55-1.51 (t, 3H).

### Preparation 13: 4-bromo-1-ethyl-3-(4-fluorophenyl)-1H-pyrazole

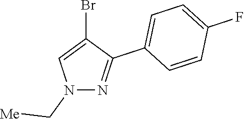 [SMILES: CCn1cc(Br)c(-c2ccc(F)cc2)n1]

A mixture of 1-ethyl-3-(4-fluorophenyl)-1H-pyrazole (Preparation 3, 2 g, 10.5 mmol), NBS (1.86 g, 10.5 mmol) in MeCN (5.00 mL) was stirred for 2 h at rt. The mixture was concentrated in vacuo and the residue was purified by silica gel column chromatography with DCM/MeOH (20/1) to give the title compound, 1.5 g, 53% as a light yellow solid. LCMS m/z=269 [M+H]⁺.

### Preparation 14: 4-bromo-1-(2,2-difluoroethyl)-3-phenyl-1H-pyrazole

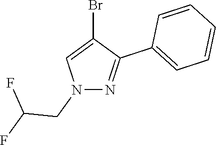 [SMILES: FC(F)Cn1cc(Br)c(-c2ccccc2)n1]

To a solution of 1-(2,2-difluoroethyl)-3-phenyl-1H-pyrazole (Preparation 2, 137 g, 658 mmol) in MeCN (1.50 L) was added NBS (117 g, 658 mmol) and the reaction was stirred at 80° C. for 1 h. The reaction mixture was concentrated under reduced pressure and the residue was purified by column chromatography (SiO₂, PE:EtOAc=20:1 to 3:1) to give the title compound, (165 g, 87.3% yield) as a brown oil. LCMS m/z=287.0 [M+H]⁺.

### Preparation 15: 4-bromo-3-(4-fluorophenyl)-1-methyl-1H-pyrazole

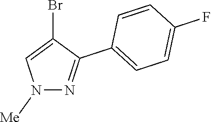 [SMILES: Cn1cc(Br)c(-c2ccc(F)cc2)n1]

NBS (968 mg, 5.44 mmol) was added to 3-(4-fluorophenyl)-1-methyl-1H-pyrazole (Preparation 8, 800 mg, 4.54 mmol) in DMF (15 mL) at rt and the resulting mixture stirred at rt for 2 h. The mixture was diluted with EtOAc (100 mL) and washed with brine (2×50 mL). The combined organics were dried (Na₂SO₄) and evaporated to dryness in vacuo. The residue was purified by column chromatography (25:1 DCM/MeOH) to afford the title compound as a yellow solid (720 mg, 90%). LCMS: m/z=255 [M+H]⁺.

### Preparation 16: 5-bromo-2-methyl-4-phenylthiazole

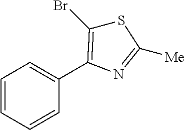 [SMILES: Cc1nc(-c2ccccc2)c(Br)s1]

The title compound was prepared as a white solid (1.3 g, 90%) from 2-methyl-4-phenylthiazole using an analogous method to that described for Preparation 15. LCMS: m/z=254 [M+H]⁺.

### Preparation 17: 4-bromo-3-(2-fluorophenyl)-1-methyl-1H-pyrazole

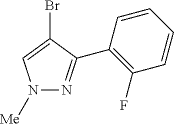 [SMILES: Cn1cc(Br)c(-c2ccccc2F)n1]

To a solution of 3-(2-fluorophenyl)-1-methyl-1H-pyrazole (Preparation 6, 637 mg, 3.62 mmol) in DMF (9 mL) was added NBS (643 mg, 3.62 mmol) portion wise over 10 mins and the reaction was stirred at rt for 3 h. The reaction was quenched with water, extracted with EtOAc, the combined organic extracts were washed with brine, then dried over anhydrous Na₂SO₄, filtered and solvent removed under reduced pressure. The crude product was purified by Combiflash ISCO (0-50% EtOAc in Hexanes) to give the title compound, as a light brown oil, 428 mg, 46% yield. LCMS m/z=255 [M+H]⁺.

### Preparation 18 to 22

The compounds in the following table were prepared from the appropriate pyrazole, following a similar procedure to that described in Preparation 17.

| Preparation No | Name, Structure, Starting Material (SM), Data |
|---|---|
| 18 | 4-bromo-3-(2,4-difluorophenyl)-1-methyl-1H-pyrazole |
| | 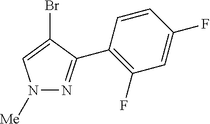 [SMILES: Cn1cc(Br)c(-c2ccc(F)cc2F)n1] |
| | SM: 3-(2,4-difluorophenyl)-1-methyl-1H-pyrazole (Preparation 9) |
| | yellow solid, 900 mg, 65%. LCMS m/z = 273 [M + H]⁺. |
| 19 | 4-bromo-3-(2,6-difluorophenyl)-1-methyl-1H-pyrazole |
| | 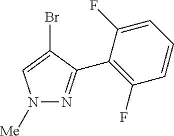 [SMILES: Cn1cc(Br)c(-c2c(F)cccc2F)n1] |
| | SM: 3-(2,6-difluorophenyl)-1-methyl-1H-pyrazole (Preparation 11) |
| | a white solid, 1.35 g, 92% yield. 1H NMR (500 MHz, CDCl₃) δ 7.96-7.88 (m, |
| | 2H), 7.70 (t, 1H), 7.49-7.35 (m, 3H), 5.52-5.39 (m, 1H), 5.08 (dt, 4H). |
| 20 | 4-bromo-1-ethyl-3-(2-fluorophenyl)-1H-pyrazole |
| | 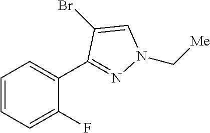 [SMILES: CCn1cc(Br)c(-c2ccccc2F)n1] |
| | SM: 1-ethyl-3-(2-fluorophenyl)-1H-pyrazole (Preparation 4) |
| | yellow oil (1.6 g, 94%). LCMS: m/z = 269 [M + H]⁺. |
| 21 | 4-bromo-3-(2,5-difluorophenyl)-1H-pyrazole |
| | 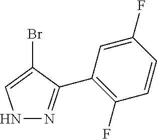 [SMILES: Fc1ccc(F)c(-c2n[nH]cc2Br)c1] |
| | SM: 3-(2,5-Difluorophenyl)-1H-pyrazole |
| | light yellow oil (650 mg, 70%). LCMS m/z = 259, 261 [M + H]⁺. |
| 22 | 4-bromo-1-(oxetan-3-yl)-3-phenyl-1H-pyrazole |
| | 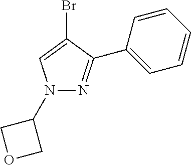 [SMILES: Brc1cn(C2COC2)nc1-c1ccccc1] |
| | SM: 1-(oxetan-3-yl)-3-phenyl-1H-pyrazole (Preparation 5) |
| | white solid, 1.03 g, 85% yield. 1H NMR (500 MHz, CDCl₃) δ 7.96-7.88 (m, |
| | 2H), 7.70 (t, 1H), 7.49-7.35 (m, 3H), 5.52-5.39 (m, 1H), 5.08 (dt, 4H). |

### Preparation 23:3-(4-bromo-1-methyl-1H-pyrazol-3-yl)phenol

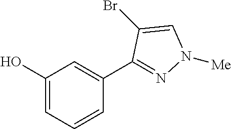 [SMILES: Cn1cc(Br)c(-c2cccc(O)c2)n1]

NBS (2.63 g, 14.8 mmol) was added to 1-methyl-3-(3-((tetrahydro-2H-pyran-2-yl)oxy)phenyl)-1H-pyrazole (Preparation 10, 3.5 g, 13.5 mmol) in THF (60 mL) at 0° C. and the mixture was stirred at rt for 2 h. The resulting solution was extracted with EtOAc (3×100 mL), the combined organics dried (Na₂SO₄) and evaporated to dryness. The residue was purified by silica gel chromatography (33% EtOAc/PE) to afford the title compound as a white solid (2.6 g, 76%). LCMS: m/z=253 [M+H]⁺.

### Preparation 24: 4-bromo-3-(2-fluorophenyl)-1H-pyrazole

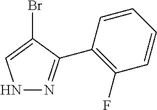 [SMILES: Fc1ccccc1-c1n[nH]cc1Br]

Pyridinium tribromide (1.67 g, 5.24 mmol) was added to 3-(2-fluorophenyl)-1H-pyrazole (850 mg, 5.24 mmol) in MeOH (9 mL) and the reaction mixture was stirred at rt for 2 h. The resulting solution was concentrated in vacuo. The residue was purified by prep-TLC (30:1 DCM/MeOH) to afford the title compound as a light yellow liquid (1.2 g, 93%). LCMS m/z=241 [M+H]⁺.

### Preparation 25: 4-bromo-3-(3-chlorophenyl)-1H-pyrazole

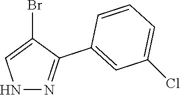 [SMILES: Clc1cccc(-c2n[nH]cc2Br)c1]

The title compound was obtained as an off-white solid, 1.36 g, 95%, from 3-(3-chlorophenyl)-1H-pyrazole following a similar procedure to that described in Preparation 24. LCMS m/z=257, 259 [M+H]⁺.

### Preparation 26: 4-bromo-3-phenyl-1-(tetrahydro-2H-pyran-2-yl)-1H-pyrazole

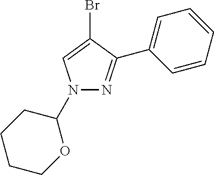 [SMILES: Brc1cn(C2CCCCO2)nc1-c1ccccc1]

To a mixture of 4-bromo-3-phenyl-1H-pyrazole (500 mg, 2.24 mmol) in DCM (30 mL) was added dihydropyran (942 mg, 11.2 mmol) and para-toluene sulfonate (385 mg, 2.24 mmol). The reaction mixture was stirred at 50° C. for 2 h. The solvent was removed in vacuo. The residue was purified by column chromatography (SiO₂, 25% EtOAc/PE) to afford the title compound as an off-white solid (480 mg, 70%). LCMS m/z=307, 309 [M+H]⁺.

### Preparation 27: 4-bromo-3-(2-fluorophenyl)-1-(tetrahydro-2H-pyran-2-yl)-1H-pyrazole

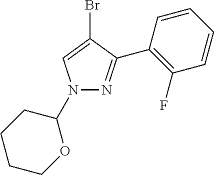 [SMILES: Fc1ccccc1-c1nn(C2CCCCO2)cc1Br]

The title compound was obtained as a colourless liquid, 750 mg, 47% yield, from 4-bromo-3-(2-fluorophenyl)-1H-pyrazole, following a similar procedure to that described for Preparation 26. LCMS m/z=325 [M+H]⁺.

### Preparation 28: 4-bromo-3-(3-chlorophenyl)-1-(tetrahydro-2H-pyran-2-yl)-1H-pyrazole

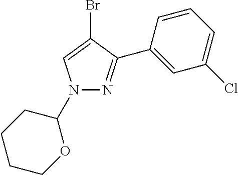 [SMILES: Clc1cccc(-c2nn(C3CCCCO3)cc2Br)c1]

The title compound was obtained as an off-white solid, 900 mg, 68% yield, from 4-bromo-3-(3-chlorophenyl)-1H-pyrazole (Preparation 25) and dihydropyran following a similar procedure to that described in Preparation 26. LCMS m/z=341, 343 [M+H]⁺.

### Preparation 29: 4-bromo-3-(2,5-difluorophenyl)-1-(tetrahydro-2H-pyran-2-yl)-1H-pyrazole

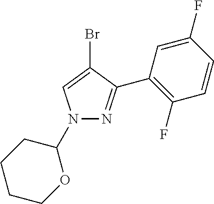 [SMILES: Fc1ccc(F)c(-c2nn(C3CCCCO3)cc2Br)c1]

The title compound was obtained as a colourless liquid, 570 mg, 67% yield, from 4-bromo-3-(2,5-difluorophenyl)-1H-pyrazole (Preparation 21), using a similar procedure to that described in Preparation 26. LCMS m/z=343, 345 [M+H]⁺.

### Preparation 30: 4-bromo-3-phenyl-1-(2,2,2-trifluoroethyl)-1H-pyrazole

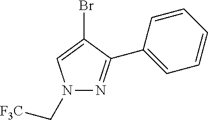 [SMILES: FC(F)(F)Cn1cc(Br)c(-c2ccccc2)n1]

A mixture of 4-bromo-3-phenyl-1H-pyrazole (1.0 g, 4.48 mmol), 2,2,2-trifluoroethyl trifluoromethanesulfonate (0.775 mL, 5.60 mmol), DMF (9 mL) and Cs₂CO₃ (2.92 g, 8.97 mmol) was stirred at rt overnight. The reaction was diluted with water and extracted with EtOAc (2×). The combined organic layers were washed with water (4×) and brine, dried over MgSO₄, filtered and concentrated. The residue was purified by silica column chromatography (DCM) to give the title compound, 894 mg, 65.4% yield. ¹H NMR (500 MHZ, DMSO-d₆) δ 8.23 (s, 1H), 7.83-7.78 (m, 2H), 7.53-7.46 (m, 2H), 7.46-7.41 (m, 1H), 5.21 (q, 2H).

### Preparation 31: 4-bromo-3-(3-((tert-butyldimethylsilyl)oxy)phenyl)-1-methyl-1H-pyrazole

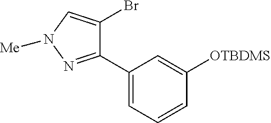 [SMILES: Cn1cc(Br)c(-c2cccc(O[Si](C)(C)C(C)(C)C)c2)n1]

Imidazole (2.07 g, 30.5 mmol) was added to 3-(4-bromo-1-methyl-1H-pyrazol-3-yl)phenol (2.6 g, 10.2 mmol) and TBSCl (4.60 g, 30.5 mmol) in DMF (50 mL) at 0° C. and the reaction mixture stirred at rt for 2 h. The resulting solution was extracted with EtOAc (3×100 mL), the combined organics dried (Na₂SO₄) and evaporated to dryness in vacuo. The residue was purified by silica gel chromatography (50% DCM/PE) to afford the title compound as a white solid (3 g, 80%). LCMS: m/z=367 [M+H]⁺.

### Preparation 32: 4-bromo-1-(2-((tert-butyldimethylsilyl)oxy)ethyl)-3-phenyl-1H-pyrazole

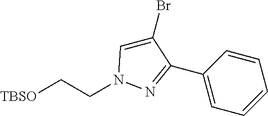 [SMILES: CC(C)(C)[Si](C)(C)OCCn1cc(Br)c(-c2ccccc2)n1]

To a solution of 4-bromo-3-phenyl-1H-pyrazole (2 g, 8.96 mmol), (2-bromoethoxy)(tert-butyl)dimethylsilane (3.20 g, 13.4 mmol) and DMF (25 mL) was added NaH (321 mg, 13.4 mmol) and the resulting mixture stirred at 50° C. for 2 h. The reaction was quenched with water and evaporated to dryness in vacuo. The residue was purified by column chromatography (5% EtOAc/PE) to afford the title compound as a yellow liquid (3.2 g, 85%). LCMS: m/z=383 [M+H]⁺.

### Preparation 33: 1-ethyl-3-phenyl-4-(4,4,5,5-tetramethyl-1,3,2-dioxaborolan-2-yl)-1H-pyrazole

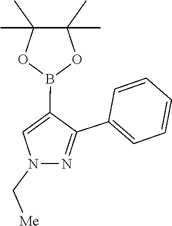 [SMILES: CCn1cc(B2OC(C)(C)C(C)(C)O2)c(-c2ccccc2)n1]

A solution of 4-bromo-1-ethyl-3-phenyl-1H-pyrazole (Preparation 12, 40 g, 159 mmol) and 2-isopropoxy-4,4,5,5-tetramethyl-1,3,2-dioxaborolane (59.3 g, 319 mmol) in THF (400 mL) was cooled to −78° C. and n-BuLi (127 mL, 2.5M) was added dropwise. The reaction was stirred at −60° C. for 1 h, then allowed to warm to rt and stirred for a further 30 min. The mixture was poured into saturated NH₄Cl (aq) solution (500 mL) at 0° C. and the solution extracted with EtOAc (500 mL×3). The combined organic extracts were washed with brine (500 mL), dried over Na₂SO₄, filtered and the filtrate evaporated under reduced pressure. The residue was purified by silica gel column (PE/EtOAc 1/0 to 4/1) to give the title compound as a yellow solid, 22.3 g, 47% yield. LCMS m/z=299 [M+H]⁺.

### Preparation 34: 3-(2,6-difluorophenyl)-1-methyl-4-(4,4,5,5-tetramethyl-1,3,2-dioxaborolan-2-yl)-1H-pyrazole

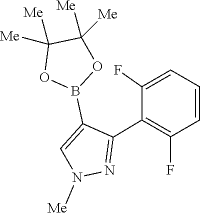 [SMILES: Cn1cc(B2OC(C)(C)C(C)(C)O2)c(-c2c(F)cccc2F)n1]

The title compound was obtained as a white solid, 364 mg, 49% from 4-bromo-3-(2,6-difluorophenyl)-1-methyl-1H-pyrazole (Preparation 19) and 2-isopropoxy-4,4,5,5-tetramethyl-1,3,2-dioxaborolane following a similar procedure to that described in Preparation 33. ¹H NMR (500 MHz, CDCl₃) δ 7.75 (t, 1H), 7.32-7.24 (m, 1H), 6.92 (td, 2H), 3.98 (t, 3H), 1.21 (t, 12H).

### Preparation 35: 1-(2-((tert-butyldimethylsilyl)oxy)ethyl)-3-phenyl-4-(4,4,5,5-tetramethyl-1,3,2-dioxaborolan-2-yl)-1H-pyrazole

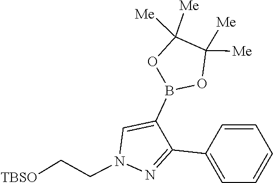 [SMILES: CC1(C)OB(c2cn(CCO[Si](C)(C)C(C)(C)C)nc2-c2ccccc2)OC1(C)C]

To a solution of 4-bromo-1-(2-((tert-butyldimethylsilyl)oxy)ethyl)-3-phenyl-1H-pyrazole (Preparation 32, 3 g, 7.76 mmol) and 2-isopropoxy-4,4,5,5-tetramethyl-1,3,2-dioxaborolane (2.15 g, 11.6 mmol) in THF (20 mL) was added BuLi with stirring at −70° C. and stirring continued for 1 h at −10° C. The reaction was quenched with water and evaporated to dryness in vacuo. The residue was purified by column chromatography to give the title compound as a yellow solid (500 mg, 13%). LCMS: m/z=429 [M+H]⁺.

### Preparation 36: 2-methyl-4-phenyl-5-(4,4,5,5-tetramethyl-1,3,2-dioxaborolan-2-yl)thiazole

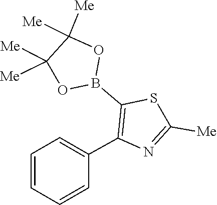 [SMILES: Cc1nc(-c2ccccc2)c(B2OC(C)(C)C(C)(C)O2)s1]

nBuLi (1.5 mL) was added to 5-bromo-2-methyl-4-phenylthiazole (Preparation 16, 1.2 g, 4.72 mmol) in THF (50 mL) at −78° C. and the resulting mixture stirred for 1 h under N₂. To this was added 4,4,5,5-tetramethyl-2-(propan-2-yloxy)-1,3,2-dioxaborolane (878 mg, 4.72 mmol) at −78° C. and the reaction mixture allowed to warm to rt and stirred at 25° C. for 2 h. The resulting solution was extracted with EtOAc (3×100 mL), the combined organics were dried (Na₂SO₄) and evaporated to dryness in vacuo. The residue was purified by column chromatography (25:1 DCM/MeOH) to afford the title compound as an off-white solid (400 mg, 28%). LCMS: m/z=254 [M+H]⁺.

### Preparation 37: 3-(2-fluorophenyl)-1-methyl-4-(4,4,5,5-tetramethyl-1,3,2-dioxaborolan-2-yl)-1H-pyrazole

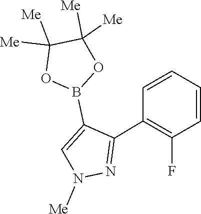 [SMILES: Cn1cc(B2OC(C)(C)C(C)(C)O2)c(-c2ccccc2F)n1]

A mixture of 4-bromo-3-(2-fluorophenyl)-1-methyl-1H-pyrazole (Preparation 17, 428 mg, 1.68 mmol), KOAc (494 mg, 5.03 mmol), B(Pin)₂ (469 mg, 1.846 mmol) and PdCl₂ (dppf) DCM (68.5 mg, 0.084 mmol) in dioxane (1.86 mL) was purged with N₂ and stirred at 100° C. for 3 h. The reaction mixture was filtered through Celite® washing through with EtOAc and the filtrate concentrated in vacuo. The crude product was purified by silica gel column (0-30% EtOAc in hexanes) to give the title compound as a sticky white solid, 220 mg, 43% yield. ¹H NMR (500 MHZ, CDCl₃) δ 7.71 (s, 1H), 7.62-7.55 (m, 1H), 7.31 (q, 1H), 7.16 (t, 1H), 7.12-7.05 (m, 1H), 3.96 (s, 3H), 1.26 (t, 12H)

### Preparation 38: 1-(oxetan-3-yl)-3-phenyl-4-(4,4,5,5-tetramethyl-1,3,2-dioxaborolan-2-yl)-1H-pyrazole

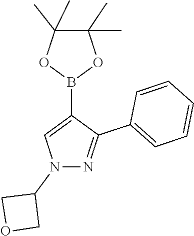 [SMILES: CC1(C)OB(c2cn(C3COC3)nc2-c2ccccc2)OC1(C)C]

The title compound was obtained as a white solid, 281 mg, 40% yield from 4-bromo-1-(oxetan-3-yl)-3-phenyl-1H-pyrazole (Preparation 22), following the procedure described in Preparation 37. ¹H NMR (500 MHz, CDCl₃) δ 8.06-7.94 (m, 3H), 7.44-7.29 (m, 3H), 5.53 (p, 1H), 5.17-5.09 (m, 2H), 5.05 (t, 2H), 1.32 (d, 12H).

### Preparation 39: 3-(3-((tert-butyldimethylsilyl)oxy)phenyl)-1-methyl-4-(4,4,5,5-tetramethyl-1,3,2-dioxaborolan-2-yl)-1H-pyrazole

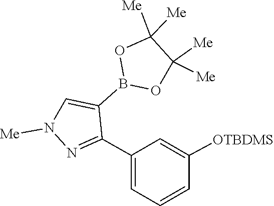 [SMILES: Cn1cc(B2OC(C)(C)C(C)(C)O2)c(-c2cccc(O[Si](C)(C)C(C)(C)C)c2)n1]

A mixture of KOAc (1.39 g, 14.1 mmol), Pd(dppf)Cl₂ (517 mg, 0.707 mmol), 4-bromo-3-(3-((tert-butyldimethylsilyl)oxy)phenyl)-1-methyl-1H-pyrazole (Preparation 31, 2.6 g, 7.07 mmol) and B(Pin)₂ (3.58 g, 14.1 mmol) in dioxane (60 mL) was stirred at 100° C. for 2 h under N₂. The reaction mixture was extracted with EtOAc (3×100 mL). The combined organics were dried (Na₂SO₄) and evaporated to dryness in vacuo. The residue was purified by silica gel chromatography (10:1 EtOAc/PE) to afford the title compound as a white solid (500 mg, 17%). LCMS: m/z=415 [M+H]⁺.

### Preparation 40: 1-ethyl-3-(2-fluorophenyl)-4-(4,4,5,5-tetramethyl-1,3,2-dioxaborolan-2-yl)-1H-pyrazole

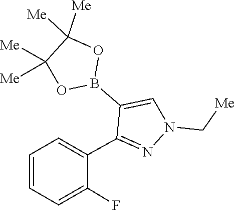 [SMILES: CCn1cc(B2OC(C)(C)C(C)(C)O2)c(-c2ccccc2F)n1]

Pd(dppf)Cl₂ (544 mg, 0.743 mmol) and KOAc (1.45 g, 14.8 mmol) were added to a solution of 4-bromo-1-ethyl-3-(2-fluorophenyl)-1H-pyrazole (Preparation 20, 2 g, 7.43 mmol) and B(Pin)₂ (3.75 g, 14.8 mmol) in DMSO and the mixture was stirred at 80° C. overnight. The reaction mixture was diluted with water (500 mL) and extracted with EtOAc (3×200 mL). The combined extracts were dried (Na₂SO₄) and evaporated to dryness in vacuo. The residue was purified by column chromatography (10:1 PE/EtOAc) to afford the title compound as an oil (2.1 g, 89%). LCMS: m/z=317 [M+H]⁺.

### Preparation 41: 1-(2,2-difluoroethyl)-3-phenyl-4-(4,4,5,5-tetramethyl-1,3,2-dioxaborolan-2-yl)-1H-pyrazole

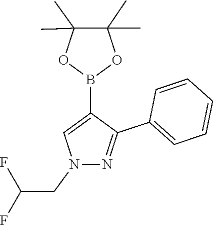 [SMILES: CC1(C)OB(c2cn(CC(F)F)nc2-c2ccccc2)OC1(C)C]

A mixture of 4-bromo-1-(2,2-difluoroethyl)-3-phenyl-1H-pyrazole (Preparation 14, 160 g, 557 mmol), (BPin)₂ (283 g, 1.11 mol), Pd(dppf)Cl₂·DCM (45.5 g, 55.7 mmol) and KOAc (164 g, 1.67 mol) in DMSO (1.80 L) was degassed and purged with N₂ (×3) and the mixture was stirred at 90° C. for 4 h under N₂. The reaction mixture was filtered, then diluted with H₂O (3.60 L) and extracted with EtOAc (2.0 L×3). The combined organic layers were washed with brine (3.0 L×2), dried over (Na₂SO₄), filtered and concentrated under reduced pressure to give a residue. The residue was purified by column chromatography (SiO₂, PE:EtOAc=20:1 to 3:1). The residue was further purified by prep-HPLC (column: Phenomenex luna c18 250 mm*100 mm*10 um; mobile phase: [water (0.225% FA)-MeCN]; B %; 50%-70%, 25 min) to give the title compound (60.0 g, 31.4% yield) as a light yellow solid. LCMS m/z=335.2 [M+H]⁺.

### Preparation 42: 3-phenyl-1-(tetrahydro-2H-pyran-2-yl)-4-(4,4,5,5-tetramethyl-1,3,2-dioxaborolan-2-yl)-1H-pyrazole

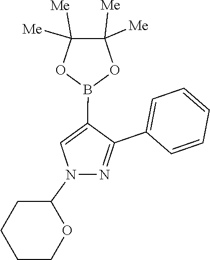 [SMILES: CC1(C)OB(c2cn(C3CCCCO3)nc2-c2ccccc2)OC1(C)C]

To a mixture of 4-bromo-3-phenyl-1-(tetrahydro-2H-pyran-2-yl)-1H-pyrazole (Preparation 26, 440 mg, 1.43 mmol) in DMSO (4 mL) was added B(Pin)₂ (726 mg, 2.86 mmol), K₃PO₄ (607 mg, 2.86 mmol) and Pd(dppf)Cl₂·DCM (116 mg, 0.143 mmol) and the reaction mixture was stirred at 100° C. for 2 h. The reaction mixture was extracted with EtOAc and the organic phase was concentrated in vacuo. The residue was purified by column chromatography (SiO₂, 10% MeOH/DCM) to afford the title compound as a light-yellow solid (400 mg, 79%). LCMS m/z=355 [M+H]⁺.

### Preparation 43: 3-(4-fluorophenyl)-1-methyl-4-(4,4,5,5-tetramethyl-1,3,2-dioxaborolan-2-yl)-1H-pyrazole

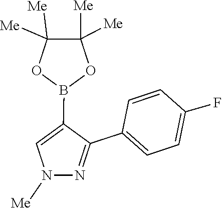 [SMILES: Cn1cc(B2OC(C)(C)C(C)(C)O2)c(-c2ccc(F)cc2)n1]

A mixture of Pd(PPh₃)₄ (216 mg, 0.282 mmol), KOAc (414 mg, 4.23 mmol), 4-bromo-3-(4-fluorophenyl)-1-methyl-1H-pyrazole (Preparation 15, 720 mg, 2.82 mmol) and B(Pin)₂ (986 mg, 3.29 mmol) in H₂O (4 mL) and DMSO (16 mL) was stirred at 100° C. for 2 h under N₂. The mixture was diluted with EtOAc (150 mL), washed with brine (2×75 mL), dried (Na₂SO₄) and evaporated to dryness in vacuo. The residue was purified by column chromatography (15:1 DCM/MeOH) to afford the title compound as a pale-yellow solid (600 mg, 83%). LCMS: m/z=303 [M+H]⁺.

### Preparation 44: 1-ethyl-3-(4-fluorophenyl)-4-(4,4,5,5-tetramethyl-1,3,2-dioxaborolan-2-yl)-1H-pyrazole

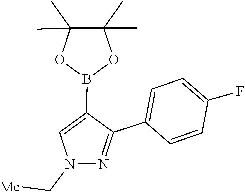 [SMILES: CCn1cc(B2OC(C)(C)C(C)(C)O2)c(-c2ccc(F)cc2)n1]

The title compound was obtained as an off-white solid, 1 g, 56.8% yield, from 4-bromo-1-ethyl-3-(4-fluorophenyl)-1H-pyrazole (Preparation 13), following the procedure described in Preparation 43. LCMS m/z=317 [M+H]⁺.

### Preparation 45: 3-(2,4-difluorophenyl)-1-methyl-4-(4,4,5,5-tetramethyl-1,3,2-dioxaborolan-2-yl)-1H-pyrazole

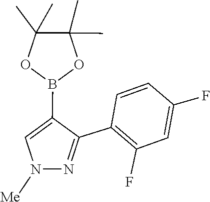 [SMILES: Cn1cc(B2OC(C)(C)C(C)(C)O2)c(-c2ccc(F)cc2F)n1]

The title compound was obtained as an off-white solid, 900 mg, from 3-(2,4-difluorophenyl)-1-methyl-1H-pyrazole (Preparation 18) following a similar procedure to that described in Preparation 43. LCMS m/z=321 [M+H]⁺

### Preparation 46: 3-(2-fluorophenyl)-1-(tetrahydro-2H-pyran-2-yl)-4-(4,4,5,5-tetramethyl-1,3,2-dioxaborolan-2-yl)-1H-pyrazole

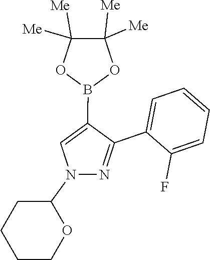 [SMILES: CC1(C)OB(c2cn(C3CCCCO3)nc2-c2ccccc2F)OC1(C)C]

The title compound was obtained as a colorless liquid, 650 mg, 76% yield, from 4-bromo-3-(2-fluorophenyl)-1-(tetrahydro-2H-pyran-2-yl)-1H-pyrazole (Preparation 27), following a similar procedure to that described in Preparation 43. LCMS m/z=373 [M+H]⁺.

### Preparation 47: 3-(3-chlorophenyl)-1-(tetrahydro-2H-pyran-2-yl)-4-(4,4,5,5-tetramethyl-1,3,2-dioxaborolan-2-yl)-1H-pyrazole

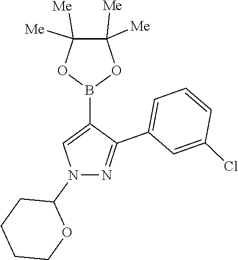 [SMILES: CC1(C)OB(c2cn(C3CCCCO3)nc2-c2cccc(Cl)c2)OC1(C)C]

The title compound was obtained as a yellow solid, 380 mg, 84%, from 4-bromo-3-(3-chlorophenyl)-1-(tetrahydro-2H-pyran-2-yl)-1H-pyrazole (Preparation 28) following a similar procedure to that described in Preparation 43. LCMS m/z=389 [M+H]⁺.

### Preparation 48: 3-(2,5-difluorophenyl)-1-(tetrahydro-2H-pyran-2-yl)-4-(4,4,5,5-tetramethyl-1,3,2-dioxaborolan-2-yl)-1H-pyrazole

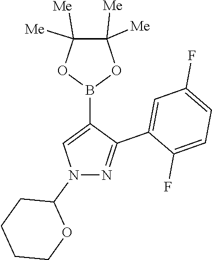 [SMILES: CC1(C)OB(c2cn(C3CCCCO3)nc2-c2cc(F)ccc2F)OC1(C)C]

A mixture of 4-bromo-3-(2,5-difluorophenyl)-1-(tetrahydro-2H-pyran-2-yl)-1H-pyrazole (Preparation 29, 300 mg, 0.874 mmol) in DMSO (3 mL), B(Pin)₂ (441 mg 1.74 mmol), Pd(PPh₃)₄ (20.1 mg, 0.0174 mmol) and NaOAc (142 mg, 1.74 mmol) was stirred at 100° C. for 16 h. The resulting solution was concentrated under reduced pressure. The residue was purified by prep-TLC (4:1 PE/EtOAc) to afford the title compound as a light yellow oil (130 mg, 38%). LCMS m/z=391 [M+H]⁺.

### Preparation 49: 1,4-dioxepan-6-ol

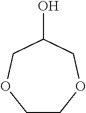

To a mixture of 1,4-dioxepan-6-one (200 mg, 1.72 mmol) in MeOH (10 mL) was added NaBH₄ (130 mg, 3.44 mmol) in ice water and the solution stirred for 10 mins. The resulting solution was diluted with water (30 mL), extracted with EtOAc (2×20 mL) and the organic layers combined. The resulting organic solution was washed with brine (20 mL), dried over anhydrous Na₂SO₄ and concentrated under vacuum to give the title compound, 150 mg, 85% yield, as a white solid.

### Preparation 50: 1-(5-methylthiazol-4-yl)ethan-1-ol

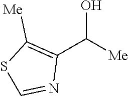

Methylmagnesium bromide (3M in Et₂O, 2 mL) was added to 5-methyl-1,3-thiazole-4-carbaldehyde (250 mg, 1.96 mmol) in THF (5 mL) at 0° C. and the reaction mixture was stirred at rt for 2 h. The reaction was quenched by the addition of 1M HCl (5 mL) and the mixture was extracted with EtOAc (3×50 mL). The combined organic layer was dried over Na₂SO₄ and evaporated under reduced pressure to give the title compound, 250 mg, 89% yield as a clear liquid. LCMS: m/z=144 [M+H]⁺.

### Preparation 51: 1-(5-methyl-1,3,4-thiadiazol-2-yl)ethan-1-ol

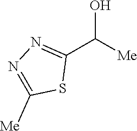

The title compound was prepared from 5-methyl-1,3,4-thiadiazole-2-carbaldehyde, following the procedure described in Preparation 50. LCMS m/z=145 [M+H]⁺.

### Preparation 52: 8-(4,4,5,5-tetramethyl-1,3,2-dioxaborolan-2-yl)-2H-benzo[b][1,4]oxazin-3(4H)-one

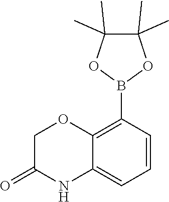 [SMILES: CC1(C)OB(c2cccc3c2OCC(=O)N3)OC1(C)C]

A mixture of 8-bromo-3,4-dihydro-2H-1,4-benzoxazin-3-one (300 mg, 1.31 mmol), (BPin)₂ (497 mg, 1.96 mmol), Pd(dppf)Cl₂ (96.0 mg, 1.96 mmol) and KOAc (192 mg, 1.96 mmol) in DMSO (15 mL) was stirred at 80° C. for 3 h under N₂. The solution was diluted with H₂O (500 mL) and extracted with EtOAc (3×200 mL). The combined organic layer was dried over Na₂SO₄ and concentrated under vacuum. The crude product was purified by Prep-TLC with PE:EtOAc=2:1 to afford the title compound (120 mg) as a brown solid. LCMS: m/z=276 [M+H]⁺.

### Preparation 53: (S)-2,4-dimethylpiperazine-1-carbonyl chloride

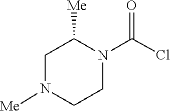 [SMILES: C[C@H]1CN(C)CCN1C(=O)Cl]

To a solution of triphosgene (59 mg, 0.20 mmol) in DCM (1 mL) at 0° C. under N₂ was added pyridine (0.142 mL, 1.75 mmol) followed by(S)-1,3-dimethylpiperazine (0.067 mL, 0.5 mmol). The reaction mixture was slowly warmed to rt and stirred overnight. The solvent was removed under reduced pressure to provide the title compound.

### Preparations 54 to 58

The compounds in the following table were obtained from the appropriate amine and phosgene, following the procedure described in Preparation above.

| Preparation No | Name, Structure, Starting Material (SM) |
|---|---|
| 54 | (3S,5R)-3,5-dimethylmorpholine-4-carbonyl chloride |
| | 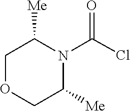 [SMILES: C[C@@H]1COC[C@H](C)N1C(=O)Cl] |
| | SM: (3S,5R)-3,5-dimethylmorpholine |
| 55 | (3S,5S)-3,5-dimethylmorpholine-4-carbonyl chloride |
| | 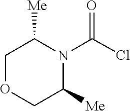 [SMILES: C[C@H]1COC[C@H](C)N1C(=O)Cl] |
| | SM: (3S,5R)-3,5-dimethylmorpholine hydrochloride |
| 56 | (2S,3S)-2,3-dimethylmorpholine-4-carbonyl chloride |
| | 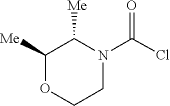 [SMILES: C[C@@H]1OCCN(C(=O)Cl)[C@H]1C] |
| | SM: (2S,3S)-2,3-dimethylmorpholine hydrochloride |
| | brown solid, 117 mg, crude |
| 57 | (R)-2-methylmorpholine-4-carbonyl chloride |
| | 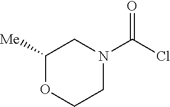 |
| | SM: (R)-2-methylmorpholine hydrochloride |
| | 400 mg, crude |
| 58 | tert-butyl (2S,5S)-4-(chlorocarbonyl)-2,5-dimethylpiperazine-1-carboxylate |
| | 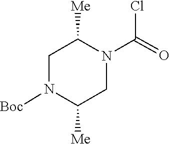 [SMILES: C[C@H]1CN(C(=O)OC(C)(C)C)[C@@H](C)CN1C(=O)Cl] |
| | SM: tert-butyl (2S,5S)-2,5-dimethylpiperazine-1-carboxylate |
| | brown oil, 1.1 g crude |

### Preparation 59: (S)-4-ethyl-2-methylpiperazine-1-carbonyl chloride

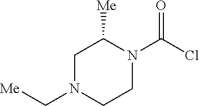 [SMILES: CCN1CCN(C(=O)Cl)[C@@H](C)C1]

Triphosgene (115 mg, 0.389 mmol) was added batchwise to pyridine (184 mg, 2.33 mmol) and (3S)-1-ethyl-3-methylpiperazine (100 mg, 0.779 mmol) in DCM (10 mL) at 0° C. and the reaction was stirred at rt for 2 h. The mixture was diluted with DCM (50 mL) and washed with brine (20 mL×2), the organic layer was dried over Na₂SO₄ and concentrated under vacuum. The residue was purified by prep-TLC with PE:EtOAc=2:1 to give the title compound, as a white solid, 100 mg.

### Preparation 60: tetrahydro-1H-pyrrolizine-7a(5H)-carboxamide

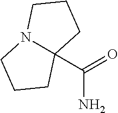 [SMILES: NC(=O)C12CCCN1CCC2]

Methyl hexahydro-1H-pyrrolizine-7a-carboxylate (200 mg, 1.18 mmol) was added to methanolic ammonia and the mixture stirred at 80° C. for 16 h. The reaction mixture was evaporated to dryness in vacuo and the residue purified by prep-TLC (20:1 DCM/MeOH=20:1) to afford the title compound as a white solid (150 mg, 82%). LCMS: m/z=155 [M+H]⁺.

### Preparation 61: ethyl 1-(2-(dimethylamino)ethyl)-1H-pyrazole-4-carboxylate

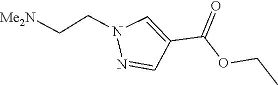

Ethyl 1H-pyrazole-4-carboxylate (300 mg, 2.14 mmol), (2-bromoethyl)dimethylamine (976 mg, 6.42 mmol) and Cs₂CO₃ (2.09 g, 6.42 mmol) were added to DMF and the reaction stirred at 60° C. for 5 h. The solution was diluted with water (500 mL) then extracted with EtOAc (3×200 mL). The organic layer was dried over Na₂SO₄ and concentrated under vacuum. The crude product was purified by column with DCM:MeOH=10:1 to afford the title compound (200 mg) as yellow oil. LCMS m/z=212 [M+H]⁺.

### Preparation 62: 1-(2-(dimethylamino)ethyl)-1H-pyrazole-4-carboxamide

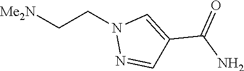 [SMILES: CN(C)CCn1cc(C(N)=O)cn1]

Ethyl 1-(2-(dimethylamino)ethyl)-1H-pyrazole-4-carboxylate (Preparation 61, 100 mg, 0.473 mmol) in NH₄OH (5 mL) was heated to 80° C. for 4 h. The mixture was concentrated under reduced pressure to afford the title compound as a yellow oil (50 mg, 58%) which was used without further purification. LCMS: m/z=183 [M+H]⁺.

### Preparation 63: tert-butyl (3-carbamoylbicyclo[1.1.1]pentan-1-yl)carbamate

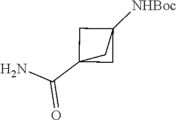 [SMILES: CC(C)(C)OC(=O)NC12CC(C(N)=O)(C1)C2]

A mixture of ethyl 3-((tert-butoxycarbonyl)amino) bicyclo[1.1.1]pentane-1-carboxylate (2 g, 8.28 mmol) in NH₄OH was stirred at 60° C. for 16 h. The solution was evaporated to dryness to afford tert-butyl (3-carbamoylbicyclo[1.1.1]pentan-1-yl)carbamate as a white solid (1.5 g) which was used without further purification.

### Preparation 64: 3-oxabicyclo[3.1.0]hexane-1-carboxamide

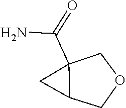

To a solution of 3-oxa-bicyclo[3.1.0]hexane-1-carboxylic acid (200 mg, 1.56 mmol) in DCM (10 mL), was added EDCl (450 mg, 2.34 mmol) and HOBt (316 mg, 4.00 mmol) and the solution was stirred for 2 h at rt. 35% aq. NH₃ (3 mL) was added and the reaction stirred at rt for 1 h then concentrated to dryness. The residue was purified by prep-TLC (5% MeOH in DCM) to afford the title compound (140 mg, yield=70%) as a white solid. LCMS: m/z=128 [M+H]⁺.

### Preparation 65: tert-butyl 1-carbamoyl-3-azabicyclo[3.1.0]hexane-3-carboxylate

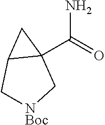 [SMILES: CC(C)(C)OC(=O)N1CC2CC2(C(N)=O)C1]

To a mixture of 3-((tert-butoxy) carbonyl)-3-azabicyclo[3.1.0]hexane-1-carboxylic acid (200 mg, 0.88 mmol) in DCM (15 mL) was added EDCl (275 mg, 1.75 mmol) and HOBt (236 mg, 1.75 mmol) and the solution stirred at 25° C. for 2 h. 35% aq. NH₃ (3 mL) was added, the reaction stirred at 25° C. for 1 h then concentrated to dryness. The residue was purified on prep-TLC with DCM:MeOH=15:1 to give the title compound (160 mg, yield: 80.4%) as a white solid. LCMS m/z=171 [M+H]⁺.

### Preparation 66: 2-methyltetrahydrofuran-2-carboxamide

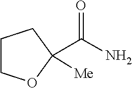

The title compound was obtained as a white solid, 150 mg, 76% yield, from 2-methyltetrahydrofuran-2-carboxylic acid, following a similar procedure to that described in Preparation 65. LCMS: m/z=130 [M+H]⁺.

### Preparation 67: (R)-2-methyltetrahydrofuran-2-carboxamide

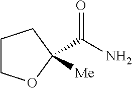

The title compound was prepared as a white solid (70 mg, 71%) from (R)-2-methyltetrahydrofuran-2-carboxylic acid using an analogous method to that described for Preparation 65. LCMS: m/z=130 [M+H]⁺.

### Preparation 68: 1-(trifluoromethyl)cyclopropane-1-carboxamide

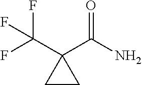

To a mixture of 1-(trifluoromethyl)cyclopropanecarboxylic acid (10 g, 64.9 mmol) in DCM (150 mL) was added HOBt (10.5 g, 77.9 mmol) and EDCl (15.0 g, 77.9 mmol) in batches. The solution was stirred at rt for 2 h then 35% aq NH₃ (5 mL) was added. The reaction was stirred at rt for 1 h then concentrated to dryness. The residue was purified on silica gel column with 5% MeOH in DCM to afford the title compound (9.1 g, yield: 91%) as a white solid. LCMS: m/z=154 [M+H]⁺.

### Preparations 69 to 75

The compounds in the following table were prepared from the appropriate carboxylic acid, following a similar procedure to that described in Preparation 68.

| Preparation No | Name, Structure, Starting carboxylic acid (RCO₂H), Data |
|---|---|
| 69 | 1-(difluoromethyl)cyclopropane-1-carboxamide |
| | 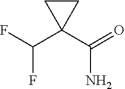 |
| | RCO₂H: 1-(difluoromethyl)cyclopropane-1-carboxylic acid |
| | white solid (260 mg, 87%) LCMS: m/z = 136 [M + H]⁺. |
| 70 | bicyclo[1.1.1]pentane-1-carboxamide |
| | 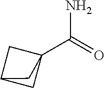 |
| | RCO₂H: bicyclo[1.1.1]pentane-1-carboxylic acid |
| | white solid, 250 g, 76%, LCMS m/z = 112 [M + H]⁺. |
| 71 | 4-methylthiazole-5-carboxamide |
| | 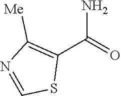 |
| | RCO₂H: 4-Methylthiazole-5-carboxylic acid |
| | off-white solid (100 mg, 75%). LCMS: m/z = 143 [M + H]⁺. |
| 72 | 1,3-dimethyl-1H-pyrazole-4-carboxamide |
| | 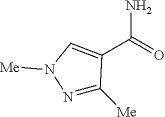 |
| | RCO₂H: 1,3-dimethyl-1H-pyrazole-4-carboxylic acid |
| | yellow oil (100 mg). LCMS: m/z = 140 [M + H]⁺. |
| 73 | tert-butyl 1-carbamoyl-6,6-difluoro-3-azabicyclo[3.1.0]hexane-3-carboxylate |
| | 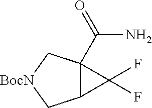 [SMILES: CC(C)(C)OC(=O)N1CC2C(F)(F)C2(C(N)=O)C1] |
| | RCO₂H: 3-(tert-butoxycarbonyl)-6,6-difluoro-3-azabicyclo[3.1.0]hexane-1- |
| | carboxylic acid |
| | white solid (400 mg, 80%). LCMS: m/z = 207 [M − 55 + H]⁺. |
| 74 | tert-butyl (1R,3S,5R)-3-carbamoyl-2-azabicyclo[3.1.0]hexane-2-carboxylate |
| | 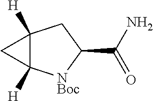 [SMILES: CC(C)(C)OC(=O)N1[C@@H]2C[C@@H]2C[C@H]1C(N)=O] |
| | RCO₂H: (1R,3S,5R)-2-(tert-butoxycarbonyl)-2-azabicyclo[3.1.0]hexane-3- |
| | carboxylic acid |
| | yellow solid (200 mg, 40%). LCMS: m/z = 227 [M + H]⁺. |
| 75 | tert-butyl (1R,3R,5R)-3-carbamoyl-2-azabicyclo[3.1.0]hexane-2-carboxylate |
| | 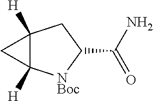 |
| | RCO₂H: (1R,3R,5R)-2-(tert-butoxycarbonyl)-2-azabicyclo[3.1.0]hexane-3- |
| | carboxylic acid |
| | yellow solid (200 mg, 40%) LCMS: m/z = 227 [M + H]⁺. |

### Preparation 76: 1-(trifluoromethyl)-1H-pyrazole-4-carboxamide

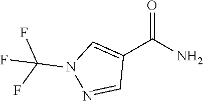 [SMILES: NC(=O)c1cnn(C(F)(F)F)c1]

A mixture of 1-(trifluoromethyl) pyrazole-4-carboxylic acid (200 mg, 1.11 mmol), HOBT (225 mg, 1.67 mmol), EDCl (319 mg, 1.67 mmol) and NH₄OH (77.8 mg, 2.22 mmol) in DCM (5 mL) was stirred for 3 h at rt. The reaction was quenched by the addition of water and the resulting solution extracted with DCM (3×10 mL). The combined organics were evaporated to dryness in vacuo and the residue purified by column chromatography (SiO₂, 20:1 DCM/MeOH) to afford the title compound as an off-white solid (150 mg, 75%).

### Preparation 77: 1-isopropyl-3-methyl-1H-pyrazole-4-carboxamide

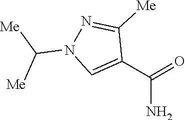 [SMILES: Cc1nn(C(C)C)cc1C(N)=O]

The title compound was obtained as a white solid, 90 mg, from 1-isopropyl-3-methylpyrazole-4-carboxylic acid following the procedure described in Preparation 76. LCMS: m/z=168 [M+H]⁺.

### Preparation 78: 2-(trifluoromethyl) pyrrolidine-2-carboxamide

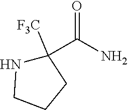 [SMILES: NC(=O)C1(C(F)(F)F)CCCN1]

LiOH·H₂O (267 mg, 6.36 mmol), H₂O (2 mL) and 30% H₂O₂ (1 mL) were added to a solution of 2-(trifluoromethyl) pyrrolidine-2-carbonitrile (300 mg, 1.82 mmol) in MeOH (6 mL) and the reaction mixture was stirred at rt for 2 h. The reaction mixture was diluted with water and extracted with EtOAc. The combined organics were dried (Na₂SO₄) and evaporated to dryness in vacuo. The residue was purified by column chromatography (2:1 EtOAc/PE) to afford the title compound as a colourless oil (140 mg, 42%). LCMS: m/z=183 [M+H]⁺.

### Preparation 79 and 80: Methyl 1-(bromodifluoromethyl)-1H-pyrazole-3-carboxylate and methyl 1-(bromodifluoromethyl)-1H-pyrazole-5-carboxylate

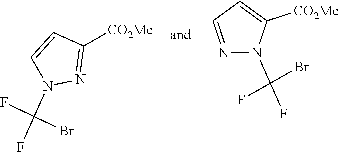 [SMILES: COC(=O)c1ccn(C(F)(F)Br)n1 ; COC(=O)c1ccnn1C(F)(F)Br]

To a mixture of methyl 1H-pyrazole-3-carboxylate (3 g, 23.7 mmol) in DMF (45 mL) was added NaH (1.42 g, 59.2 mmol) at 0° C. and the reaction mixture stirred at 0° C. for 15 min. ibromodifluoromethane (14.9 g, 71.1 mmol) was added and the reaction stirred at 25° C. for 16 h. The reaction mixture was added to ice water and extracted with EtOAc. The combined organics were evaporated to dryness in vacuo and the residue was purified by column chromatography (14% EtOAc/PE) to give the title compounds.

Peak 1, Preparation 79; methyl 1-(bromodifluoromethyl)-1H-pyrazole-3-carboxylate (colourless oil, 3 g, 50%). LCMS: m/z=255 [M+H]⁺; ¹H NMR (300 MHz, DMSO-d₆) δ: 8.61 (dt, 1H), 7.06 (d, 1H), 3.87 (s, 4H).

Peak 2, Preparation 80; methyl 1-(bromodifluoromethyl)-1H-pyrazole-5-carboxylate (colourless oil, 400 mg, 7%). LCMS: m/z=255 [M+H]⁺; ¹H NMR (300 MHz, DMSO-d₆) δ 8.02 (d, 1H), 7.26 (dt, 1H), 3.89 (s, 3H).

### Preparation 81: Methyl 1-(trifluoromethyl)-1H-pyrazole-5-carboxylate

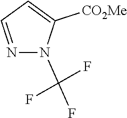 [SMILES: COC(=O)c1ccnn1C(F)(F)F]

To a mixture of methyl 1-(bromodifluoromethyl)-1H-pyrazole-5-carboxylate (Preparation 80, 400 mg, 1.56 mmol) in DCM (20 mL) was added AgBF₄ (912 mg, 4.68 mmol) at −78° C. under N₂ atmosphere and the reaction was stirred overnight at rt. The resulting solution was diluted with water (40 mL) and extracted with DCM (2×50 mL). The combined organics were washed with brine (30 mL), dried (Na₂SO₄) and evaporated to dryness in vacuo. The residue was purified by column chromatography (10% EtOAc/PE) to afford the title compound as a yellow oil (300 mg, 99%). LCMS: m/z=195 [M+H]⁺.

### Preparation 82: 1-(trifluoromethyl)-1H-pyrazole-5-carboxylic acid

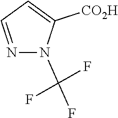 [SMILES: O=C(O)c1ccnn1C(F)(F)F]

To a solution of methyl 1-(trifluoromethyl)-1H-pyrazole-5-carboxylate (Preparation 81, 300 mg, 1.54 mmol) in THF/H₂O was added LiOH (110 mg, 4.62 mmol) and the mixture stirred at 25° C. for 4 h. The resulting solution was evaporated to dryness in vacuo to afford the title compound as a white solid (150 mg, 54%) which was used without further purification.

### Preparation 83: Methyl 1-(bromodifluoromethyl)-3-methyl-1H-pyrazole-4-carboxylate and methyl 1-(bromodifluoromethyl)-5-methyl-1H-pyrazole-4-carboxylate

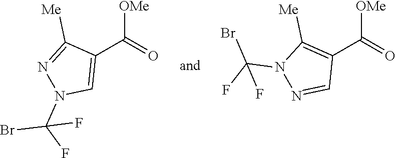 [SMILES: COC(=O)c1cn(C(F)(F)Br)nc1C ; COC(=O)c1cnn(C(F)(F)Br)c1C]

NaH (567 mg, 14.2 mmol) was added to methyl 3-methyl-1H-pyrazole-4-carboxylate (2 g, 14.2 mmol) in DMF (10 mL) at 0° C. and then stirred at 0° C. for 30 min before CF₂Br₂ (4.43 g, 21.3 mmol) was added and the resulting mixture stirred at rt for 16 h. The mixture was evaporated to dryness in vacuo and the residue purified by prep-TLC (2:1 PE/EtOAc) to give a mixture of the title compounds as a pale-yellow solid (1.9 g, 50%). LCMS: m/z=269 [M+H]⁺.

### Preparation 84: Methyl 3-methyl-1-(trifluoromethyl)-1H-pyrazole-4-carboxylate and methyl 5-methyl-1-(trifluoromethyl)-1H-pyrazole-4-carboxylate

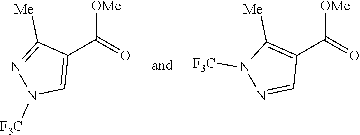 [SMILES: COC(=O)c1cn(C(F)(F)F)nc1C ; COC(=O)c1cnn(C(F)(F)F)c1C]

AgBF₄ (2.88 g, 14.8 mmol) was added to mixture of methyl 1-(bromodifluoromethyl)-3-methyl-1H-pyrazole-4-carboxylate and methyl 1-(bromodifluoromethyl)-5-methyl-1H-pyrazole-4-carboxylate (Preparation 83, 2 g, 7.43 mmol) in DCM (30 mL) at −78° C. under N₂ and the resulting mixture stirred at rt for 16 h. The reaction mixture was filtered and the filtrate was concentrated in vacuo. The residue was purified by prep-TLC (50% EtOAc/PE) to afford a mixture of the title compounds as an off-white oil (540 mg). LCMS: m/z=209 [M+H]⁺.

### Preparation 85: 3-methyl-1-(trifluoromethyl)-1H-pyrazole-4-carboxylic acid and 5-methyl-1-(trifluoromethyl)-1H-pyrazole-4-carboxylic Acid

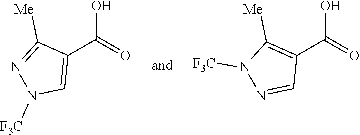 [SMILES: Cc1nn(C(F)(F)F)cc1C(=O)O ; Cc1c(C(=O)O)cnn1C(F)(F)F]

LiOH (343 mg, 14.3 mmol) was added to mixture of 3-methyl-1-(trifluoromethyl)-1H-pyrazole-4-carboxylate and methyl 5-methyl-1-(trifluoromethyl)-1H-pyrazole-4-carboxylate (Preparation 84, 500 mg, 2.40 mmol) in THF/H₂O (15 mL/5 mL) at rt and the resulting mixture was stirred at rt for 16 h. The reaction mixture was diluted with water and the pH adjusted to pH 6˜7 with HCl (1 M) and extracted with EtOAc (100 mL). The combined organics were washed with water (3×100 mL) and brine (100 mL), then dried (Na₂SO₄) and evaporated to dryness in vacuo. The residue was purified by column chromatography (20:1 DCM/MeOH) to afford the title compounds as a white solid (430 mg, 92%). LCMS: m/z=195 [M+H]⁺.

### Preparation 86: 7-methoxy-4-(1-methyl-3-phenyl-1H-pyrazol-4-yl)quinazolin-6-ol

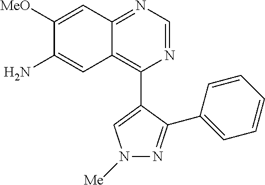 [SMILES: COc1cc2ncnc(-c3cn(C)nc3-c3ccccc3)c2cc1N]

A mixture of 4-chloro-7-methoxyquinazolin-6-ol (2.0 g, 9.49 mmol), 1-methyl-3-phenyl-4-(4,4,5,5-tetramethyl-1,3,2-dioxaborolan-2-yl)-1H-pyrazole (3.21 g, 11.3 mmol), Pd(dppf)Cl₂ (1.38 g, 1.89 mmol) and K₃PO₄ (4.0 g, 18.9 mmol) in dioxane and H₂O was stirred for 2 h at 100° C. under N₂. The cooled mixture was partitioned between EtOAc and water, the layers separated and the aqueous phase extracted with EtOAc. The combined organic layers were dried over Na₂SO₄ and concentrated in vacuo. The residue was purified by silica gel column chromatography eluting with DCM/MeOH (20:1) to give the title compound as a yellow solid, 2.20 g, 69.8%. LCMS m/z=333 [M+H]⁺.

### Preparation 87: 6-bromo-7-fluoroquinazolin-4 (3H)-one

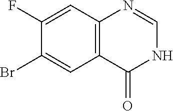 [SMILES: O=c1[nH]cnc2cc(F)c(Br)cc12]

A mixture of 2-amino-5-bromo-4-fluorobenzoic acid (2 g, 8.54 mmol) and formamidine acetate (4.43 g, 42.6 mmol) in EtOH (20 mL) was stirred at 100° C. for 16 h. the mixture was concentrated to dryness, the residue diluted with water and extracted with DCM. The combined organic layer was dried over Na₂SO₄, filtered and the filtrate was evaporated under reduced pressure to give the title compound (2 g, yield=96%) as an off-white solid. LCMS m/z=243, 245 [M+H]⁺.

### Preparation 88: 6-bromo-7-ethoxyquinazolin-4 (3H)-one

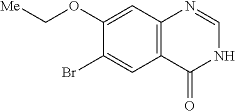 [SMILES: CCOc1cc2nc[nH]c(=O)c2cc1Br]

To a solution of EtONa in EtOH (21%, 10 mL), was added 6-bromo-7-fluoroquinazolin-4 (3H)-one (Preparation 87, 2 g, 8.22 mmol) and the reaction was stirred at 80° C. for 16 h. The reaction was concentrated to dryness, the residue was diluted with water and the mixture filtered. The solid was dried in vacuo to afford title compound (2.1 g, yield=90%) as a white solid. LCMS m/z=271 [M+H]⁺.

### Preparation 89: 6-bromo-4-chloro-7-ethoxyquinazoline

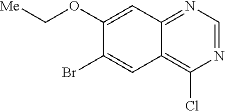 [SMILES: CCOc1cc2ncnc(Cl)c2cc1Br]

A solution of 6-bromo-7-ethoxyquinazolin-4 (3H)-one (Preparation 88, 2.2 g, 8.17 mmol) in POCl₃ (10 mL) was stirred at 100° C. for 3 h. The cooled mixture was concentrated to dryness and the residue was diluted with DCM (5 mL). The resulting solution was added drop wise to sat. Na₂CO₃ (aq)(30 mL) and the mixture extracted with DCM. The organic layer was dried over Na₂SO₄, filtered and concentrated to dryness to afford title compound (2.4 g, yield=90%) as a brown solid. LCMS m/z=289 [M+H]⁺.

### Preparation 90: 6-bromo-7-ethoxy-4-(1-methyl-3-phenyl-1H-pyrazol-4-yl)quinazoline

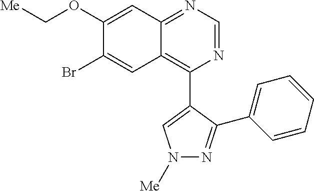 [SMILES: CCOc1cc2ncnc(-c3cn(C)nc3-c3ccccc3)c2cc1Br]

A mixture of 6-bromo-4-chloro-7-ethoxyquinazoline (Preparation 89, 300 mg, 1.04 mmol), 1-methyl-3-phenyl-4-(4,4,5,5-tetramethyl-1,3,2-dioxaborolan-2-yl)-1H-pyrazole (443 mg, 1.56 mmol), K₃PO₄ (331 mg, 1.56 mmol) and Pd(PPh₃)₄ (48 mg, 0.0416 mmol) in dioxane (15 mL) and water (5 mL) was stirred at 80° C. for 3 h. The reaction mixture was evaporated to dryness and the residue purified by prep-TLC (20:1 DCM/MeOH) to afford the title compound as a yellow solid (225 mg, 53%). LCMS: m/z=409, 411 [M+H]⁺.

### Preparation 91: 6-bromo-7-methoxy-4-(1-methyl-3-phenyl-1H-pyrazol-4-yl)quinazoline

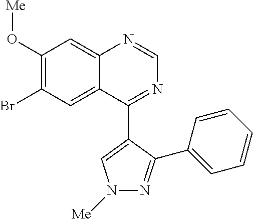 [SMILES: COc1cc2ncnc(-c3cn(C)nc3-c3ccccc3)c2cc1Br]

The title compound was obtained as a white solid, 1.0 g, 49%, from 6-bromo-4-chloro-7-methoxyquinazoline and 1-methyl-3-phenyl-4-(4,4,5,5-tetramethyl-1,3,2-dioxaborolan-2-yl)-1H-pyrazole, following a similar procedure to that described in Preparation 90. LCMS m/z=397 [M+H]⁺.

### Preparation 92: 6-bromo-4-(3-(2,6-difluorophenyl)-1-methyl-1H-pyrazol-4-yl)-7-methoxy quinazoline

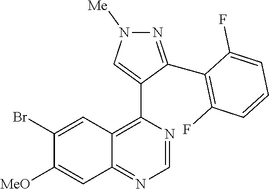 [SMILES: COc1cc2ncnc(-c3cn(C)nc3-c3c(F)cccc3F)c2cc1Br]

The title compound was prepared as an off-white solid (160 mg, 88%) from 6-bromo-4-chloro-7-methoxyquinazoline and 3-(2,6-difluorophenyl)-1-methyl-4-(4,4,5,5-tetramethyl-1,3,2-dioxaborolan-2-yl)-1H-pyrazole (Preparation 34) using an analogous method to that described for Preparation 90. LCMS: m/z=397 [M+H]⁺.

### Preparation 93: 6-bromo-7-ethoxy-4-(1-ethyl-3-phenyl-1H-pyrazol-4-yl)quinazoline

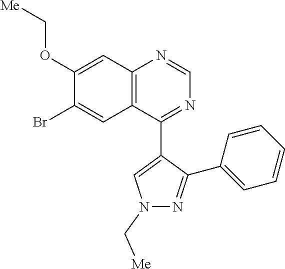 [SMILES: CCOc1cc2ncnc(-c3cn(CC)nc3-c3ccccc3)c2cc1Br]

The title compound was obtained as an off-white solid, 180 mg, 61% yield, from 6-bromo-4-chloro-7-ethoxyquinazoline (Preparation 89) and 1-ethyl-3-phenyl-4-(4,4,5,5-tetramethyl-1,3,2-dioxaborolan-2-yl)-1H-pyrazole (Preparation 33), following a similar procedure to that described in Preparation 90. LCMS m/z=423,425 [M+H]⁺.

### Preparation 94: 6-bromo-4-(1-ethyl-3-phenyl-1H-pyrazol-4-yl)-7-methoxyquinazoline

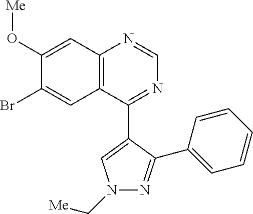 [SMILES: CCn1cc(-c2ncnc3cc(OC)c(Br)cc23)c(-c2ccccc2)n1]

The title compound was obtained as a yellow solid, 200 mg, 57% yield, from 6-bromo-4-chloro-7-methoxyquinazoline and 1-ethyl-3-phenyl-4-(4,4,5,5-tetramethyl-1,3,2-dioxaborolan-2-yl)-1H-pyrazole (Preparation 33), following the procedure described in Preparation 90. LCMS m/z=409,411 [M+H]⁺.

### Preparation 95: 6-bromo-7-ethoxy-4-(3-phenyl-1-(tetrahydro-2H-pyran-2-yl)-1H-pyrazol-4-yl)quinazoline

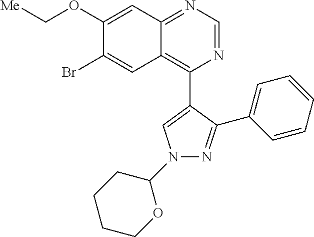 [SMILES: CCOc1cc2ncnc(-c3cn(C4CCCCO4)nc3-c3ccccc3)c2cc1Br]

The title compound was obtained as a yellow solid, 560 mg, 56% yield, from 6-bromo-4-chloro-7-ethoxyquinazoline (Preparation 89) and 3-phenyl-1-(tetrahydro-2H-pyran-2-yl)-4-(4,4,5,5-tetramethyl-1,3,2-dioxaborolan-2-yl)-1H-pyrazole (Preparation 42), following a similar procedure to that described in Preparation 90. LCMS m/z=479,481 [M+H]⁺.

### Preparation 96: 6-bromo-4-(3-(2,4-difluorophenyl)-1-methyl-1H-pyrazol-4-yl)-7-methoxy quinazoline

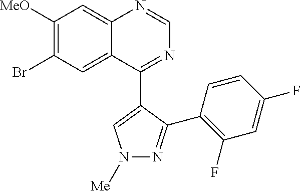 [SMILES: COc1cc2ncnc(-c3cn(C)nc3-c3ccc(F)cc3F)c2cc1Br]

Pd(PPh₃)₄ (95.2 mg, 0.124 mmol) and K₃PO₄ (394 mg, 1.86 mmol) were added to 3-(2,4-difluorophenyl)-1-methyl-4-(4,4,5,5-tetramethyl-1,3,2-dioxaborolan-2-yl)-1H-pyrazole (Preparation 45, 400 mg, 1.24 mmol) and 6-bromo-4-chloro-7-methoxyquinazoline (404 mg, 1.48 mmol) in H₂O (4 mL) and dioxane (16 mL) and the reaction mixture was stirred at 80° C. for 2 h under N₂. The mixture was diluted with DCM (100 mL), washed with brine (50 mL×2), the organic layer was dried over Na₂SO₄ and concentrated under vacuum. The residue was purified by silica gel column with DCM:MeOH=20:1 to give the title compound, 400 mg as a white solid. LCMS m/z=431 [M+H]⁺.

### Preparation 97: 6-bromo-4-(3-(4-fluorophenyl)-1-methyl-1H-pyrazol-4-yl)-7-methoxyquinazoline

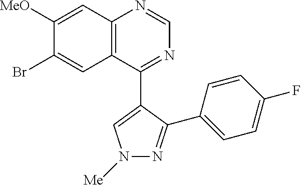 [SMILES: COc1cc2ncnc(-c3cn(C)nc3-c3ccc(F)cc3)c2cc1Br]

A mixture of Pd(dppf)Cl₂ (465 mg, 0.571 mmol), K₃PO₄ (419 mg, 1.98 mmol), 3-(4-fluorophenyl)-1-methyl-4-(4,4,5,5-tetramethyl-1,3,2-dioxaborolan-2-yl)-1H-pyrazole (Preparation 43, 400 mg, 1.32 mmol) and 6-bromo-4-chloro-7-methoxyquinazoline (432 mg, 1.58 mmol) in H₂O (6 mL) and dioxane (24 mL) was stirred at 80° C. for 2 h under N₂. The mixture was diluted with EtOAc (150 mL), washed with brine (2×75 mL), dried (Na₂SO₄) and evaporated to dryness in vacuo. The residue was purified by column chromatography (15:1 DCM/MeOH) to afford the title compound as an off-white solid (205 mg, 51%). LCMS: m/z=413 [M+H]⁺.

### Preparation 98: 6-bromo-4-(1-ethyl-3-(2-fluorophenyl)-1H-pyrazol-4-yl)-7-methoxyquinazoline

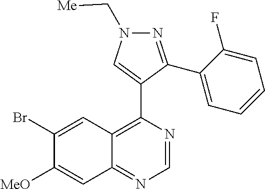 [SMILES: CCn1cc(-c2ncnc3cc(OC)c(Br)cc23)c(-c2ccccc2F)n1]

Pd(dppf)Cl₂ (133 mg, 1.82 mmol) and K₂CO₃ (251 mg, 1.82 mmol) were added to a solution of 6-bromo-4-chloro-7-methoxyquinazoline (500 mg, 1.82 mmol) and 1-ethyl-3-(2-fluorophenyl)-4-(4,4,5,5-tetramethyl-1,3,2-dioxaborolan-2-yl)-1H-pyrazole (Preparation 40, 575 mg, 1.82 mmol) in dioxane/H₂O and the mixture was stirred at 80° C. for 3 h. The resulting solution was extracted with EtOAc (3×20 mL), dried (Na₂SO₄) and evaporated to dryness in vacuo. The residue was purified by prep-TLC (30:1 DCM/MeOH) to afford the title compound as a yellow solid (290 mg, 37%). LCMS: m/z=427 [M+H]⁺.

### Preparation 99: 6-bromo-4-(1-ethyl-3-phenyl-1H-pyrazol-4-yl)quinazolin-7-ol

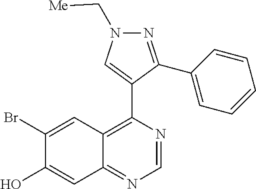 [SMILES: CCn1cc(-c2ncnc3cc(O)c(Br)cc23)c(-c2ccccc2)n1]

A mixture of 6-bromo-4-(1-ethyl-3-phenyl-1H-pyrazol-4-yl)-7-methoxyquinazoline (Preparation 94, 400 mg, 0.977 mmol) and pyridine hydrochloride (2.25 g, 19.5 mmol) was stirred at 140° C. for 8 h. The mixture was diluted with DCM (100 mL), washed with brine (2×50 mL), dried (Na₂SO₄) and evaporated to dryness in vacuo. The residue was purified by prep-TLC (25:1 DCM/MeOH) to afford the title compound as a yellow solid (200 mg, 52%). LCMS: m/z=395 [M+H]⁺.

### Preparation 100: 6-bromo-7-(difluoromethoxy)-4-(1-ethyl-3-phenyl-1H-pyrazol-4-yl)quinazoline

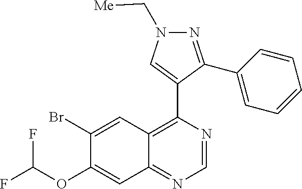 [SMILES: CCn1cc(-c2ncnc3cc(OC(F)F)c(Br)cc23)c(-c2ccccc2)n1]

Sodium 2-chloro-2,2-difluoroacetate (76.9 mg, 0.505 mmol) was added to a mixture of K₂CO₃ (69.6 mg, 0.505 mmol), 6-bromo-4-(1-ethyl-3-phenyl-1H-pyrazol-4-yl)quinazolin-7-ol (Preparation 99, 200 mg, 0.505 mmol) in DMF (10 mL) and H₂O (1 mL) at rt and the resulting mixture was heated to 100° C. for 16 h. The mixture was diluted with EtOAc (100 mL) and washed with brine (2×50 mL). The combined organics were dried (Na₂SO₄) and evaporated to dryness in vacuo. The residue was purified by prep-TLC (25:1 DCM/MeOH) to afford the title compound as a white solid (120 mg, 53%). LCMS: m/z=395 [M+H]⁺.

### Preparation 101: 6-bromo-4-(1-methyl-3-phenyl-1H-pyrazol-4-yl)quinazolin-7-ol

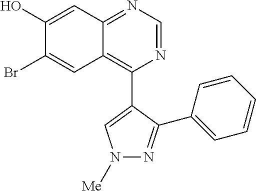 [SMILES: Cn1cc(-c2ncnc3cc(O)c(Br)cc23)c(-c2ccccc2)n1]

A solution of 6-bromo-7-methoxy-4-(1-methyl-3-phenyl-1H-pyrazol-4-yl)quinazoline (Preparation 91, 300 mg, 0.759 mmol) in BBr₃ (10.40 g, 41.51 mmol) was stirred at 80° C. for 6 h. The mixture was added dropwise to H₂O (30 mL) at 0° C., the pH adjusted with Na₂CO₃ to pH 8-9 and the mixture was extracted with DCM (20 mL×3). The combined organic layers were dried over Na₂SO₄, filtered and concentrated under reduced pressure. The residue was purified by prep-TLC (PE/EtOAc=1/1) to give the title compound, 74 mg, 25.6% as a yellow solid.

### Preparation 102: 6-bromo-7-isopropoxy-4-(1-methyl-3-phenyl-1H-pyrazol-4-yl)quinazoline

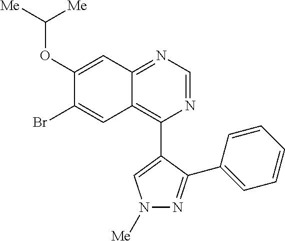 [SMILES: CC(C)Oc1cc2ncnc(-c3cn(C)nc3-c3ccccc3)c2cc1Br]

To a solution of 6-bromo-4-(1-methyl-3-phenyl-1H-pyrazol-4-yl)quinazolin-7-ol (Preparation 101, 45 mg, 0.118 mmol) and 2-iodopropane (30.1 mg, 0.177 mmol) in DMF (3 mL) was added K₂CO₃ (32.63 mg, 0.236 mmol) and the reaction was stirred at 80° C. for 2 h. The reaction mixture was diluted with H₂O (5 mL) and extracted with EtOAc (3 mL×3). The combined organic layers were washed with brine (5 mL×2), dried over Na₂SO₄, filtered and concentrated under reduced pressure. The residue was purified by prep-TLC (0 to 100% EtOAc in PE) to give the title compound, 27 mg, 54% as a white solid.

### Preparation 103: 7-isopropoxy-4-(1-methyl-3-phenyl-1H-pyrazol-4-yl)quinazolin-6-ol

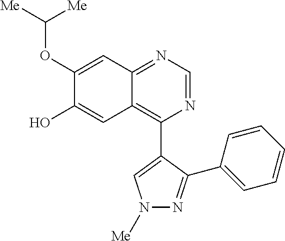 [SMILES: CC(C)Oc1cc2ncnc(-c3cn(C)nc3-c3ccccc3)c2cc1O]

To a solution of 6-bromo-7-isopropoxy-4-(1-methyl-3-phenyl-1H-pyrazol-4-yl)quinazoline (Preparation 102, 37 mg, 0.087 mmol) in dioxane (1 mL) and H₂O (0.25 mL) was added Pd₂(dba)₃ (8.0 mg, 0.009 mmol), t-BuXphos (7.42 mg, 0.0175 mmol) and KOH (9.81 mg, 0.175 mmol) and the reaction was stirred at 80° C. for 2 h under N₂. The reaction mixture was concentrated under reduced pressure and the residue was purified by prep-TLC (EtOAc/MeOH=10/1) to give the title compound, 16.0 mg, 50.8% as a yellow solid.

### Preparation 104: tert-butyl (2-((6-bromo-4-(1-methyl-3-phenyl-1H-pyrazol-4-yl)quinazolin-7-yl)oxy)ethyl)carbamate

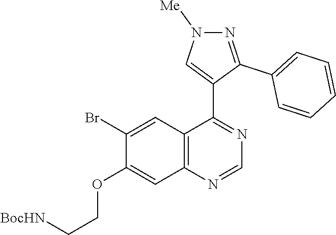 [SMILES: Cn1cc(-c2ncnc3cc(OCCNC(=O)OC(C)(C)C)c(Br)cc23)c(-c2ccccc2)n1]

A mixture of 6-bromo-4-(1-methyl-3-phenyl-1H-pyrazol-4-yl)quinazolin-7-ol (Preparation 101, 150 mg, 0.393 mmol), tert-butyl N-(2-bromoethyl)carbamate (176 mg, 0.786 mmol) and Cs₂CO₃ (257 mg, 0.786 mmol) in DMF (5 ml) was stirred at 100° C. for 16 h. The reaction was diluted with water and extracted with EtOAc (3×50 ml). The combined organics were dried (Na₂SO₄) and evaporated to dryness in vacuo and the residue purified by prep-TLC (10:1 DCM/MeOH) to afford the title compound as a light yellow solid (180 mg, 87%). LCMS: m/z=523 [M+H]⁺.

### Preparation 105: 2-((6-bromo-4-(1-methyl-3-phenyl-1H-pyrazol-4-yl)quinazolin-7-yl)oxy)-N,N-dimethylethan-1-amine

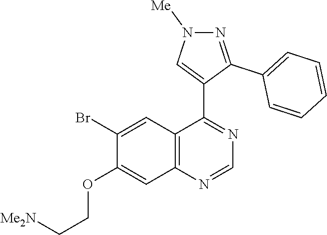 [SMILES: CN(C)CCOc1cc2ncnc(-c3cn(C)nc3-c3ccccc3)c2cc1Br]

Part 1. TFA was added to tert-butyl (2-((6-bromo-4-(1-methyl-3-phenyl-1H-pyrazol-4-yl)quinazolin-7-yl)oxy)ethyl)carbamate (Preparation 104, 200 mg, 0.381 mmol) in DCM and the mixture stirred at rt for 4 h. The reaction was evaporated to dryness in vacuo to afford 2-((6-bromo-4-(1-methyl-3-phenyl-1H-pyrazol-4-yl)quinazolin-7-yl)oxy) ethan-1-amine which was used in Part 2 without purification.

Part 2. HCHO solution (173 μL, 1.76 mmol) was added to a solution of 2-((6-bromo-4-(1-methyl-3-phenyl-1H-pyrazol-4-yl)quinazolin-7-yl)oxy) ethan-1-amine (Part 2, 150 mg, 0.354 mmol) in DCM followed by Na(OAc)₃BH (224 mg, 1.06 mmol) and the reaction mixture stirred at rt for 1 h. The reaction was quenched by NH₄Cl solution and extracted with DCM. The combined organics were evaporated to dryness and the residue purified by prep-TLC (10:1 DCM/MeOH) to afford the title compound as a white solid (70 mg, 41%). LCMS: m/z=454 [M+H]⁺.

### Preparation 106: 6-bromo-4-(1-(2-((tert-butyldimethylsilyl)oxy)ethyl)-3-phenyl-1H-pyrazol-4-yl)-7-methoxyquinazoline

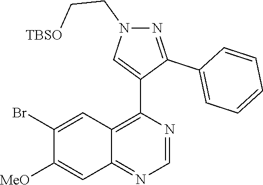 [SMILES: COc1cc2ncnc(-c3cn(CCO[Si](C)(C)C(C)(C)C)nc3-c3ccccc3)c2cc1Br]

A mixture of 1-(2-((tert-butyldimethylsilyl)oxy)ethyl)-3-phenyl-4-(4,4,5,5-tetramethyl-1,3,2-dioxaborolan-2-yl)-1H-pyrazole (Preparation 35, 390 mg, 0.774 mmol), 6-bromo-4-chloro-7-methoxyquinazoline (317 mg, 1.16 mmol), Pd(PPh₃)₄ (30 mg, 0.026 mmol) and K₃PO₄ (326 mg, 1.54 mmol) in dioxane (12 mL) and H₂O (3 mL) was stirred at 80° C. for 2 h. The reaction mixture was evaporated to dryness and the residue purified by prep-TLC (35:1 DCM/MeOH to afford the title compound as a yellow solid (240 mg, 52%). LCMS: m/z=541 [M+H]⁺.

### Preparation 107: 6-bromo-7-methoxy-4-(3-phenyl-1H-pyrazol-4-yl)quinazoline

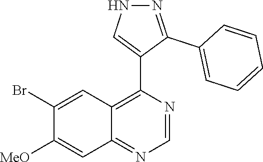 [SMILES: COc1cc2ncnc(-c3c[nH]nc3-c3ccccc3)c2cc1Br]

To a solution of 6-bromo-4-(1-(2-((tert-butyldimethylsilyl)oxy)ethyl)-3-phenyl-1H-pyrazol-4-yl)-7-methoxyquinazoline (Preparation 106, 570 mg, 1.10 mmol) in DCM (20 mL) was added TFA (5 mL) and the reaction stirred at rt for 2 h. The reaction was concentrated to dryness, the residue was diluted with DCM and washed with sat. aq. Na₂CO₃. The organic layer was separated, dried over Na₂SO₄, filtered and concentrated to dryness to afford the title compound (480 mg, yield: 84.9%) as a yellow solid. LCMS m/z=381, 383 [M+H]⁺

### Preparation 108: 6-bromo-7-methoxy-4-(3-phenyl-1-propyl-1H-pyrazol-4-yl)quinazoline

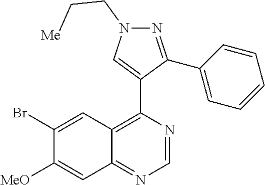 [SMILES: CCCn1cc(-c2ncnc3cc(OC)c(Br)cc23)c(-c2ccccc2)n1]

NaH (31.3 mg, 0.786 mmol) was added to a solution of 6-bromo-7-methoxy-4-(3-phenyl-1H-pyrazol-4-yl)quinazoline (Preparation 107, 150 mg, 0.393 mmol), 1-bromopropane (96.6 mg, 0.786 mmol) and DMF (5 mL) and the mixture stirred at 50° C. for 2 h. The reaction was quenched with water and evaporated to dryness. The residue was purified by prep-TLC (20:1 DCM/MeOH) to afford the title compound as a yellow solid (120 mg, 38%). LCMS: m/z=425 [M+H]⁺.

### Preparation 109: 7-ethoxy-4-(1-methyl-3-phenyl-1H-pyrazol-4-yl)quinazolin-6-ol

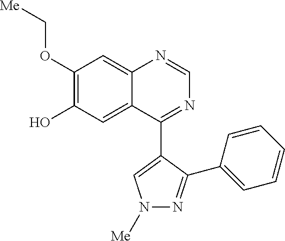 [SMILES: CCOc1cc2ncnc(-c3cn(C)nc3-c3ccccc3)c2cc1O]

A mixture of 6-bromo-7-ethoxy-4-(1-methyl-3-phenyl-1H-pyrazol-4-yl)quinazoline (Preparation 90, 500 mg, 1.22 mmol), BrettPhos Pd G3 (111 mg, 0.122 mmol) and Cs₂CO₃ (598 mg, 1.83 mmol) in H₂O (2 mL) and dioxane (10 mL) was stirred at 100° C. for 16 h. The reaction mixture was diluted with EtOAc (100 mL) and washed with brine (2×50 mL). The organic layer was dried over Na₂SO₄ and concentrated in vacuo. The residue was purified by prep-TLC (15:1 DCM/MeOH) to afford the title compound as a yellow solid (350 mg, 83%). LCMS m/z=347 [M+H]⁺.

### Preparation 110: 7-methoxy-4-(3-phenyl-1-(tetrahydro-2H-pyran-2-yl)-1H-pyrazol-4-yl)quinazolin-6-ol

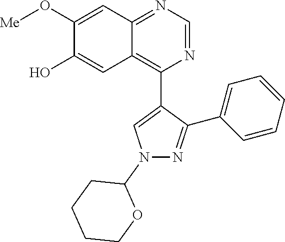 [SMILES: COc1cc2ncnc(-c3cn(C4CCCCO4)nc3-c3ccccc3)c2cc1O]

A mixture of 4-chloro-7-methoxyquinazolin-6-ol (315 mg, 1.5 mmol), 3-phenyl-1-(tetrahydro-2H-pyran-2-yl)-4-(4,4,5,5-tetramethyl-1,3,2-dioxaborolan-2-yl)-1H-pyrazole (Preparation 42, 634 mg, 1.79 mmol), Pd(dppf)₂Cl₂ (109 mg, 0.150 mmol) and K₃PO₄ (1.27 g, 6.0 mmol) in dioxane (9 mL) and H₂O (3 mL) was heated to 80° C. for 3 h. The reaction mixture was concentrated to dryness and the residue was purified by column chromatography to afford the title compound as a light brown syrup (400 mg, 66%). LCMS m/z=403 [M+H]⁺.

### Preparation 111: 4-(1-(2,2-difluoroethyl)-3-phenyl-1H-pyrazol-4-yl)-7-methoxyquinazolin-6-ol

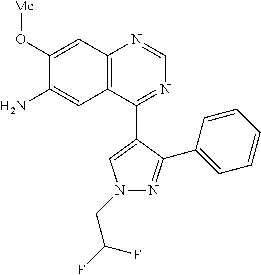 [SMILES: COc1cc2ncnc(-c3cn(CC(F)F)nc3-c3ccccc3)c2cc1N]

A mixture of 4-chloro-7-methoxyquinazolin-6-ol (3 g, 14.2 mmol), 1-(2,2-difluoroethyl)-3-phenyl-4-(4,4,5,5-tetramethyl-1,3,2-dioxaborolan-2-yl)-1H-pyrazole (Preparation 41, 5.68 g, 17.0 mmol) Pd(PPh₃)₄ (1.64 g, 1.42 mmol) and K₃PO₄ (3.60 g, 17.0 mmol) in dioxane/H₂O, was stirred at 80° C. for 16 h. The resulting solution was extracted with EtOAc (3×20 mL). The combined organic layers were dried over Na₂SO₄ and concentrated in vacuo. The crude product was purified by column chromatography (SiO₂, 3% MeOH/DCM) to afford the title compound as a yellow solid (2.4 g, 44%). LCMS m/z=383 [M+H]⁺.

### Preparation 112: 4-(3-(2-fluorophenyl)-1-methyl-1H-pyrazol-4-yl)-7-methoxyquinazolin-6-ol

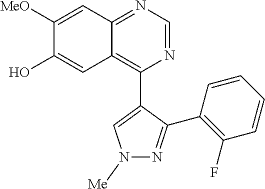 [SMILES: COc1cc2ncnc(-c3cn(C)nc3-c3ccccc3F)c2cc1O]

To a mixture of 4-chloro-7-methoxyquinazolin-6-ol (70 mg, 0.332 mmol), K₂CO₃ (92 mg, 0.665 mmol) and 3-(2-fluorophenyl)-1-methyl-4-(4,4,5,5-tetramethyl-1,3,2-dioxaborolan-2-yl)-1H-pyrazole (Preparation 37, 151 mg, 0.499 mmol) in water (0.317 mL), dioxane (1.266 mL) and DME (0.633 mL) was added Pd(PPh₃)₄ (38.4 mg, 0.033 mmol) and the reaction was stirred at 100° C. for 5 h under N₂. The mixture was filtered through Celite® eluting with DCM/MeOH and the mixture was concentrated in vacuum. The residue was purified by Combiflash Isco, 12 g gold column, 0-20% MeOH in DCM, to give the title compound, as a viscous yellow oil, 92.4 mg, 63.5% yield.

### Preparation 113: 4-(3-(2,6-difluorophenyl)-1-methyl-1H-pyrazol-4-yl)-7-methoxyquinazolin-6-ol

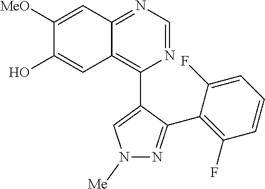 [SMILES: COc1cc2ncnc(-c3cn(C)nc3-c3c(F)cccc3F)c2cc1O]

The title compound was obtained as an off-white solid, 40.6 mg, 46.4% yield from 4-chloro-7-methoxyquinazolin-6-ol and 3-(2,6-difluorophenyl)-1-methyl-4-(4,4,5,5-tetramethyl-1,3,2-dioxaborolan-2-yl)-1H-pyrazole (Preparation 34), following the procedure described in Preparation 112.

### Preparation 114: 7-methoxy-4-(1-(oxetan-3-yl)-3-phenyl-1H-pyrazol-4-yl)quinazolin-6-ol

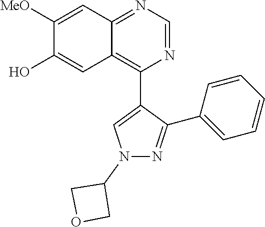 [SMILES: COc1cc2ncnc(-c3cn(C4COC4)nc3-c3ccccc3)c2cc1O]

The title compound was obtained as a light yellow solid, 89.6 mg, 36% yield, from 1-(oxetan-3-yl)-3-phenyl-4-(4,4,5,5-tetramethyl-1,3,2-dioxaborolan-2-yl)-1H-pyrazole (Preparation 38) and 4-chloro-7-methoxyquinazolin-6-ol, following the procedure described in Preparation 112.

### Preparation 115: 7-methoxy-4-(3-phenyl-1-(2,2,2-trifluoroethyl)-1H-pyrazol-4-yl)quinazolin-6-ol

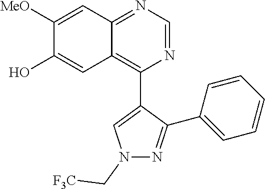 [SMILES: COc1cc2ncnc(-c3cn(CC(F)(F)F)nc3-c3ccccc3)c2cc1O]

A mixture of 4-bromo-3-phenyl-1-(2,2,2-trifluoroethyl)-1H-pyrazole (Preparation 30, 250 mg, 0.819 mmol), (BPin)₂ (250 mg, 0.983 mmol), KOAc (241 mg, 2.458 mmol) and PdCl₂ (dppf)-DCM (100 mg, 0.123 mmol) in dioxane (4 mL) was sparged with N₂ for 5 mins and the reaction was heated at 90° C. overnight. The mixture was cooled to rt, 4-chloro-7-methoxyquinazolin-6-ol (86 mg, 0.410 mmol), 2M aq. K₂CO₃ (1.229 mL, 2.46 mmol) and additional PdCl₂(dppf)-DCM (100 mg, 0.123 mmol) were added and the reaction heated at 90° C. overnight. The reaction was diluted with water (1 mL) and mixed vigorously. The resulting solution was added to an Isolute HMN SPE tube (5 mL size) and allowed to gravity elute with EtOAc. The filtrate was concentrated and dried under vacuum. The residue was purified by silica column chromatography (DCM to 20% MeOH in DCM) to give the title compound, 142 mg, 39.0%. LCMS m/z=401.1 [M+H]⁺.

### Preparation 116: 7-methoxy-4-(1-methyl-3-phenyl-1H-pyrazol-4-yl)quinazolin-6-yl trifluoromethanesulfonate

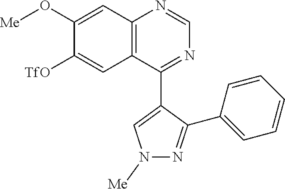 [SMILES: COc1cc2ncnc(-c3cn(C)nc3-c3ccccc3)c2cc1OS(=O)(=O)C(F)(F)F]

Tf₂O (2.11 g, 7.50 mmol) was added dropwise to 7-methoxy-4-(1-methyl-3-phenyl-1H-pyrazol-4-yl)quinazolin-6-ol (Preparation 86, 1 g, 3.0 mmol) and TEA (1.21 g, 12.0 mmol) in dry DCM (20 mL) at −60° C. and the reaction was stirred for 1 h. The solution was extracted with DCM (2×50 mL), the combined organic layers were washed with brine (50 mL), dried over Na₂SO₄ and concentrated in vacuo. The residue was purified by column chromatography (SiO₂, 1:1 DCM/EtOAc) to afford the title compound as a yellow solid (1.0 g, 72%). LCMS m/z=465 [M+H]⁺.

### Preparation 117: 4-(1-(2,2-difluoroethyl)-3-phenyl-1H-pyrazol-4-yl)-7-methoxyquinazolin-6-yl trifluoromethanesulfonate

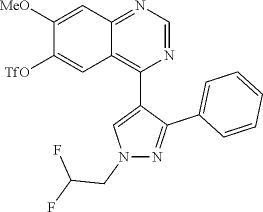 [SMILES: COc1cc2ncnc(-c3cn(CC(F)F)nc3-c3ccccc3)c2cc1OS(=O)(=O)C(F)(F)F]

TEA (116 mg, 1.15 mmol) was added to an ice-cooled solution of 4-(1-(2,2-difluoroethyl)-3-phenyl-1H-pyrazol-4-yl)-7-methoxyquinazolin-6-ol (Preparation 111, 220 mg, 0.575 mmol) in DCM (20 mL). Tf₂O was added dropwise and the reaction stirred for 1 h. The reaction was neutralized with aq. NaHCO₃, extracted with DCM (60 mL×3) and the combined organic extracts were concentrated in vacuo. The crude compound was purified by TLC PE:EtOAc=1:1 to give the title compound, 250 mg, as a red solid. LCMS m/z=515 [M+H]⁺.

### Preparation 118: 4-(3-(2-fluorophenyl)-1-methyl-1H-pyrazol-4-yl)-7-methoxyquinazolin-6-yl trifluoromethanesulfonate

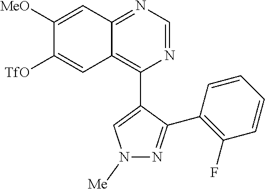 [SMILES: COc1cc2ncnc(-c3cn(C)nc3-c3ccccc3F)c2cc1OS(=O)(=O)C(F)(F)F]

To a solution of 4-(3-(2-fluorophenyl)-1-methyl-1H-pyrazol-4-yl)-7-methoxyquinazolin-6-ol (Preparation 112, 200 mg, 0.570 mmol) in DCM (15 mL) was added TEA (230 mg, 2.28 mmol) and the solution cooled to −50° C. Tf₂O (482 mg, 1.71 mmol) was added and the reaction stirred for 1 h at −50° C. The mixture was concentrated under vacuum, the residue was diluted with water (100 mL), the mixture was extracted with EtOAc (2×100 mL) and the organic layers combined. The organic solution was washed with brine (50 mL), dried over anhydrous Na₂SO₄ and concentrated under vacuum. The product was purified by chromatography with DCM:MeOH (10:1) to give the title compound, 240 mg, 87.5%, as yellow solid. LCMS m/z=483 [M+H]⁺.

### Preparation 119: tert-butyl (3R,4R)-3-fluoro-4-((7-methoxy-4-(1-methyl-3-phenyl-1H-pyrazol-4-yl)quinazolin-6-yl)oxy) piperidine-1-carboxylate

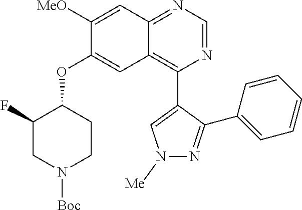 [SMILES: COc1cc2ncnc(-c3cn(C)nc3-c3ccccc3)c2cc1O[C@@H]1CCN(C(=O)OC(C)(C)C)C[C@H]1F]

DIAD (484 mg, 2.40 mmol) was added dropwise to PPh₃ (789 mg, 3.0 mmol), 7-methoxy-4-(1-methyl-3-phenyl-1H-pyrazol-4-yl)quinazolin-6-ol (Preparation 86, 200 mg, 0.60 mmol) and tert-butyl (3R,4S)-3-fluoro-4-hydroxypiperidine-1-carboxylate (526 mg, 2.40 mmol) in THF at −10° C. and the resulting mixture was stirred at rt for 8 h. The mixture was diluted with EtOAc (100 mL) and washed with brine (50 mL×2). The organic layer was dried over Na₂SO₄ and concentrated under vacuum. The residue was purified by prep-TLC eluting with PE:EtOAc=1:1 to afford the title compound, 250 mg as a yellow solid. LCMS m/z=534 [M+H]⁺.

### Preparations 120 to 124

The compounds in the table below were prepared from the appropriate quinazolin-6-ol and alcohol, following a similar procedure to that described in Preparation 119.

| Preparation | |
|---|---|
| No | Name/Structure/Starting materials (SM)/Data |
| 120 | tert-butyl (3S,4S)-3-fluoro-4-((7- |
| | methoxy-4-(1-methyl-3-phenyl-1H- |
| | pyrazol-4-yl)quinazolin-6-yl)oxy)piperidine-1-carboxylate |
| | 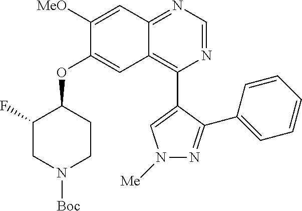 [SMILES: COc1cc2ncnc(-c3cn(C)nc3-c3ccccc3)c2cc1O[C@H]1CCN(C(=O)OC(C)(C)C)C[C@@H]1F] |
| | SM: 7-methoxy-4-(1-methyl-3-phenyl-1H-pyrazol-4-yl) |
| | quinazolin-6-ol (Preparation 86) and tert-butyl (3S,4R)-3- |
| | fluoro-4-hydroxypiperidine-1-carboxylate |
| | yellow solid, 250 mg, 62% yield. LCMS m/z = 534 [M + H]⁺ |
| 121 | Cis-rac-tert-butyl (3S,4R)-3-fluoro-4- |
| | ((7-methoxy-4-(1-methyl-3-phenyl- |
| | 1H-pyrazol-4-yl)quinazolin-6-yl)oxy)piperidine-1-carboxylate |
| | 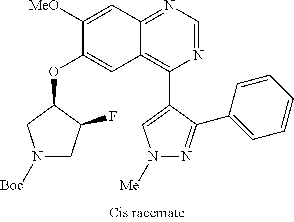 [SMILES: COc1cc2ncnc(-c3cn(C)nc3-c3ccccc3)c2cc1O[C@@H]1CN(C(=O)OC(C)(C)C)C[C@@H]1F] |
| | SM: 7-methoxy-4-(1-methyl-3-phenyl-1H-pyrazol-4-yl) |
| | quinazolin-6-ol (Preparation 86) and trans-rac-tert-butyl (3R,4R)- |
| | 3-fluoro-4-hydroxypyrrolidine-1-carboxylate |
| | yellow solid, 360 mg, 85% yield. LCMS m/z = 520 [M + H]⁺ |
| 122 | Trans-rac-tert-butyl (3S,4S)-3-fluoro- |
| | 4-((7-methoxy-4-(1-methyl-3-phenyl- |
| | 1H-pyrazol-4-yl)quinazolin-6-yl)oxy)pyrrolidine-1-carboxylate |
| | 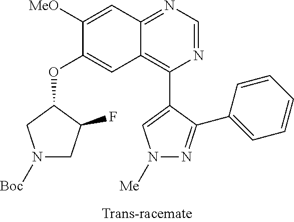 [SMILES: COc1cc2ncnc(-c3cn(C)nc3-c3ccccc3)c2cc1O[C@H]1CN(C(=O)OC(C)(C)C)C[C@@H]1F] |
| | SM: 7-methoxy-4-(1-methyl-3-phenyl-1H-pyrazol-4-yl) |
| | quinazolin-6-ol (Preparation 86) and cis-rac-tert-butyl (3S,4R)- |
| | 3-fluoro-4-hydroxypyrrolidine-1-carboxylate |
| | white solid, 400 mg, 85% yield. LCMS m/z = 520 [M + H]⁺ |
| 123 | tert-butyl (3R,4R)-4-((4-(1-(2,2-difluoroethyl)- |
| | 3-phenyl-1H-pyrazol-4-yl)- |
| | 7-methoxyquinazolin-6-yl)oxy)-3-fluoropiperidine-1-carboxylate |
| | 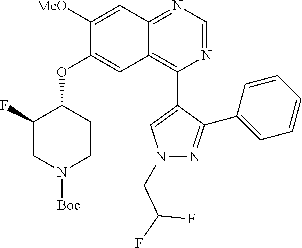 [SMILES: COc1cc2ncnc(-c3cn(CC(F)F)nc3-c3ccccc3)c2cc1O[C@@H]1CCN(C(=O)OC(C)(C)C)C[C@H]1F] |
| | SM: 4-(1-(2,2-difluoroethyl)-3-phenyl-1H-pyrazol-4-yl)-7- |
| | methoxyquinazolin-6-ol (Preparation 111) and tert-butyl |
| | (3R,4S)-3-fluoro-4-hydroxypiperidine-1-carboxylate. |
| | Yellow solid, 200 mg, 44% yield. LCMS m/z = 584 [M + H]⁺ |
| 124 | tert-butyl 4-((7-ethoxy-4-(1-methyl-3- |
| | phenyl-1H-pyrazol-4-yl)quinazolin- |
| | 6-yl)oxy)piperidine-1-carboxylate |
| | 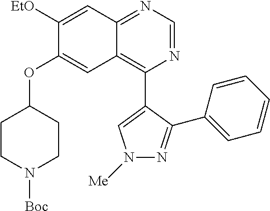 [SMILES: CCOc1cc2ncnc(-c3cn(C)nc3-c3ccccc3)c2cc1OC1CCN(C(=O)OC(C)(C)C)CC1] |
| | SM: 7-ethoxy-4-(1-methyl-3-phenyl- |
| | 1H-pyrazol-4-yl)quinazolin-6-ol |
| | (Preparation 109) and tert-butyl 4-hydroxypiperidine-1- |
| | carboxylate |
| | yellow solid, 100 mg, 65.7% yield |

### Preparation 125: 4-chloro-7-methoxy-6-((tetrahydrofuran-3-yl)oxy)quinazoline

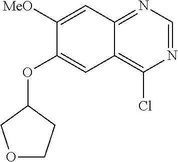 [SMILES: COc1cc2ncnc(Cl)c2cc1OC1CCOC1]

A mixture of 4-chloro-7-methoxyquinazolin-6-ol (200 mg, 0.949 mmol), PPh₃ (495 mg, 1.89 mmol) and tetrahydrofuran-3-ol (166 mg, 1.89 mmol) in THF (20 mL) was stirred at rt for 10 min. DIAD (382 mg, 1.89 mmol) was added and the reaction was stirred at rt for 4 h. The reaction was quenched with water and the mixture concentrated to dryness. The residue was purified by prep-TLC eluting with PE:EtOAc=1:1 to afford the title compound (200 mg, yield: 75%) as an off-white solid. LCMS m/z=281 [M+H]⁺.

### Preparation 126: 4-chloro-7-methoxy-6-((tetrahydro-2H-pyran-4-yl)oxy)quinazoline

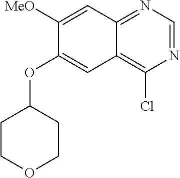 [SMILES: COc1cc2ncnc(Cl)c2cc1OC1CCOCC1]

The title compound was obtained as a colorless oil, 240 mg, 86% yield, from 4-chloro-7-methoxyquinazolin-6-ol and tetrahydro-2H-pyran-4-ol, following the procedure described in Preparation 125. LCMS m/z=295 [M+H]⁺.

### Preparation 127: Trans-rac-4-chloro-6-(((3S,4S)-3-fluorotetrahydro-2H-pyran-4-yl)oxy)-7-methoxyquinazoline

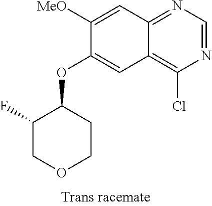 [SMILES: COc1cc2ncnc(Cl)c2cc1O[C@H]1CCOC[C@@H]1F]

PPh₃ (1.24 g, 4.74 mmol) and DIAD (773 mg, 3.79 mmol) were added to 4-chloro-7-methoxyquinazolin-6-ol (200 mg, 0.949 mmol) and cis-rac-(3S,4SR)-3-fluorotetrahydro-2H-pyran-4-ol (569 mg, 4.74 mmol) in THF (5 mL) at 0° C. and the reaction mixture was stirred at rt for 2 h under N₂. The mixture was extracted with EtOAc (3×40 mL), the combined organic layer was dried over Na₂SO₄ and concentrated under vacuum. The crude product was purified by prep-TLC with DCM:MeOH=25:1 to give the title compound as a yellow solid, 110 mg, 37%. LCMS: m/z=313 [M+H]⁺.

### Preparation 128:6-methoxy-4-(1-methyl-3-phenyl-1H-pyrazol-4-yl)quinazolin-7-ol

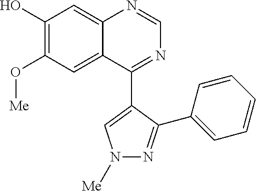 [SMILES: COc1cc2c(-c3cn(C)nc3-c3ccccc3)ncnc2cc1O]

Pd(dppf)Cl₂ (173 mg, 0.237 mmol) and K₂CO₃ (489 mg, 3.55 mmol) were added to 4-chloro-6-methoxyquinazolin-7-ol (500 mg, 2.37 mmol) and 1-methyl-3-phenyl-4-(4,4,5,5-tetramethyl-1,3,2-dioxaborolan-2-yl)-1H-pyrazole in dioxane/H₂O (4/1 V/V). The reaction was stirred at 80° C. under N₂ overnight. The resulting solution was extracted with EtOAc (3×50 mL). The combined organic layers were dried over Na₂SO₄ and concentrated in vacuo. The crude product was purified by prep-TLC (25:1 DCM/MeOH) to afford the title compound as a yellow solid (209 mg, 27%). LCMS m/z=333 [M+H]⁺.

### Preparation 129: 4-chloro-7-ethoxy-6-methoxyquinazoline

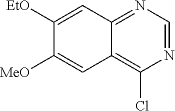 [SMILES: CCOc1cc2ncnc(Cl)c2cc1OC]

DIAD (1.15 g, 5.68 mmol) was added dropwise to PPh₃ (1.86 g, 7.10 mmol), 4-chloro-6-methoxyquinazolin-7-ol (300 mg, 1.42 mmol) and EtOH (327 mg, 7.10 mmol) in THF (15 mL) at 0° C. and the reaction stirred at rt for 2 h. The resulting solution was extracted with EtOAc (3×50 mL), the combined organic layer was dried over Na₂SO₄ and concentrated under vacuum. The crude product was purified by prep-TLC with DCM:MeOH=25:1 to give the title compound, 200 mg (59%) as a yellow solid. LCMS m/z=239 [M+H]⁺.

### Preparation 130: 4-chloro-6-methoxy-7-(2-methoxyethoxy)quinazoline

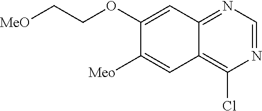

The title compound was obtained as a yellow solid, 500 mg, 56% yield, from 4-chloro-6-methoxyquinazolin-7-ol and 2-methoxyethan-1-ol following the procedure described in Preparation 129. LCMS m/z=269 [M+H]⁺.

### Preparation 131: 6-methoxy-4-(1-methyl-3-phenyl-1H-pyrazol-4-yl)quinazolin-7-ol

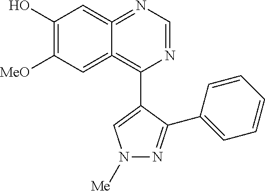

To a solution of 4-chloro-6-methoxyquinazolin-7-ol (350 mg, 1.66 mmol) in dioxane/H₂O (4/1 V/V) was added 1-methyl-3-phenyl-4-(4,4,5,5-tetramethyl-1,3,2-dioxaborolan-2-yl)-1H-pyrazole (565 mg, 1.99 mmol), K₂CO₃ (458 mg, 3.32 mmol) and Pd(dppf)Cl₂ (121 mg, 0.166 mmol) under N₂ and the reaction was stirred at 80° C. for 2 h. The reaction mixture was cooled to rt, then diluted with water (20 mL). The resulting solution was extracted with EtOAc (2×20 mL), the organic layers combined, washed with brine (20 mL), dried over anhydrous Na₂SO₄ and concentrated under vacuum. The crude product was purified by prep-TLC (DCM:MeOH=10:1) to give the title compound, 270 mg, 49.0%, as a yellow solid. LCMS m/z=333 [M+H]⁺.

### Preparation 132: 4-(1-methyl-3-phenyl-1H-pyrazol-4-yl)quinazoline-6,7-diol

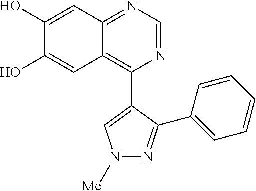 [SMILES: Cn1cc(-c2ncnc3cc(O)c(O)cc23)c(-c2ccccc2)n1]

To a solution of 6-methoxy-4-(1-methyl-3-phenyl-1H-pyrazol-4-yl)quinazolin-7-ol (Preparation 131, 140 mg, 0.421 mmol) in DCE (5 mL) was added BBr₃ (1.06 g, 4.21 mmol) dropwise at 0° C. The mixture was stirred at 25° C. for 1 h, then quenched with H₂O (10 mL) and extracted with DCM (10 mL×2). The aqueous layer was diluted with MeOH (20 mL), the mixture was filtered and the filtrate was concentrated under reduced pressure to give the title compound (150 mg, crude) as a yellow solid.

### Preparation 133: 6-(benzyloxy)-7-bromopyrido[3,2-d]pyrimidin-4-ol

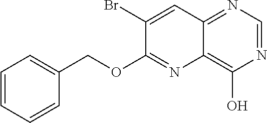 [SMILES: Oc1ncnc2cc(Br)c(OCc3ccccc3)nc12]

To a solution of benzyl alcohol (177.26 mg, 1.64 mmol) in DMF (2.0 mL) was added NaH (98.35 mg, 2.46 mmol, 60% purity) and the mixture was stirred at 25° C. for 5 mins. 7-Bromo-6-fluoropyrido[3,2-d]pyrimidin-4-ol (400 mg, 1.64 mmol) was added and the reaction was stirred at 25° C. for 20 mins. The reaction was diluted with H₂O (8 mL) at 0° C., and then 1M HCl added dropwise until pH=3. The reaction mixture was filtered and the filter cake was concentrated under reduced pressure to give the title compound as a white solid.

### Preparation 134: 6-(benzyloxy)-7-methoxypyrido[3,2-d]pyrimidin-4-ol

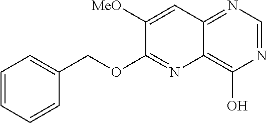 [SMILES: COc1cc2ncnc(O)c2nc1OCc1ccccc1]

To a solution of 6-(benzyloxy)-7-bromopyrido[3,2-d]pyrimidin-4-ol (Preparation 133, 200 mg, 0.602 mmol) in MeOH (5.0 mL) and DMSO (5.0 mL) was added t-BuXphos (25.57 mg, 0.06 mmol), Cs₂CO₃ (392.37 mg, 1.20 mmol) and Pd₂(dba)₃ (55.14 mg, 0.06 mmol) and the mixture was stirred at 80° C. for 2 h under N₂. The crude product was diluted with H₂O (8 mL) at 0° C., and then 1M HCl added dropwise until pH=3. The reaction mixture was filtered and the filtrate was freeze-dried. The crude product was purified by re-crystallization from H₂O (2 mL) to give the title compound, as an off-white solid.

### Preparation 135: 6-(benzyloxy)-4-chloro-7-methoxypyrido[3,2-d]pyrimidine

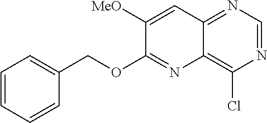 [SMILES: COc1cc2ncnc(Cl)c2nc1OCc1ccccc1]

To a solution of 6-(benzyloxy)-7-methoxypyrido[3,2-d]pyrimidin-4-ol (Preparation 134, 100 mg, 0.353 mmol) in CCl₄ (1.0 mL) and DCE (2.0 mL) was added PPh₃ (277.8 mg, 1.06 mmol) and the reaction was stirred at 75° C. for 2 h under N₂. The reaction mixture was concentrated under reduced pressure and the residue was purified by column chromatography (PE/EtOAc=1/0 to 1/1) to give the title compound (150 mg, crude) as a yellow gum.

### Preparation 136: 6-(benzyloxy)-7-methoxy-4-(1-methyl-3-phenyl-1H-pyrazol-4-yl)pyrido[3,2-d]pyrimidine

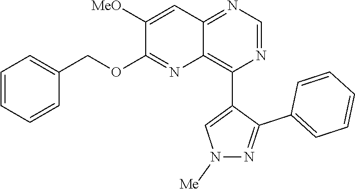 [SMILES: COc1cc2ncnc(-c3cn(C)nc3-c3ccccc3)c2nc1OCc1ccccc1]

To a solution of 6-(benzyloxy)-4-chloro-7-methoxypyrido[3,2-d]pyrimidine (Preparation 135, 150 mg, 0.497 mmol) and 1-methyl-3-phenyl-4-(4,4,5,5-tetramethyl-1,3,2-dioxaborolan-2-yl) pyrazole (70.63 mg, 0.249 mmol) in H₂O (0.10 mL), DME (1.00 mL) and dioxane (2.00 mL) was added Pd(PPh₃)₄ (57.45 mg, 0.05 mmol) and K₂CO₃ (137.41 mg, 0.994 mmol). The mixture was stirred at 100° C. for 1 hr under N₂. The reaction mixture was concentrated under reduced pressure to give a residue. The residue was purified by prep-TLC (PE/EtOAc=0/1) to give the title compound (40 mg, 19.0% yield) as a white solid. ¹H-NMR (400 MHz, CDCl₃) δ ppm 9.05 (s, 1H), 7.97 (s, 1H), 7.49-7.47 (m, 2H), 7.41 (s, 1H), 7.37-7.34 (m, 5H), 7.26-7.24 (m, 3H), 5.06 (s, 2H), 4.03 (s, 3H), 4.01 (s, 3H).

### Preparation 137: 7-methoxy-4-(1-methyl-3-phenyl-1H-pyrazol-4-yl)pyrido[3,2-d]pyrimidin-6-ol

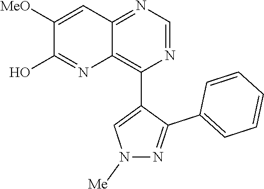 [SMILES: COc1cc2ncnc(-c3cn(C)nc3-c3ccccc3)c2nc1O]

To a solution of 6-(benzyloxy)-7-methoxy-4-(1-methyl-3-phenyl-1H-pyrazol-4-yl)pyrido[3,2-d]pyrimidine (Preparation 136, 40.0 mg, 0.095 mmol) in EtOAc (5.0 mL) was added Pd/C (40.0 mg, 10% purity, 50% in H₂O) and the reaction was stirred at 25° C. for 16 h under H₂ (15 psi). The reaction mixture was filtered and the filtrate was concentrated under reduced pressure to the title compound (40.0 mg, crude) as a yellow solid.

### Preparation 138: 7-methoxy-4-(3-phenyl-1-(tetrahydro-2H-pyran-2-yl)-1H-pyrazol-4-yl)quinazolin-6-yl (2S)-2,4-dimethylpiperazine-1-carboxylate

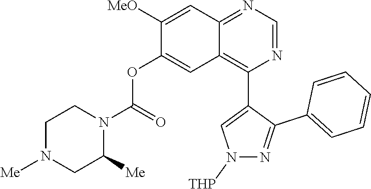

A mixture of 7-methoxy-4-(3-phenyl-1-(tetrahydro-2H-pyran-2-yl)-1H-pyrazol-4-yl)quinazolin-6-ol (Preparation 110, 100 mg, 0.248 mmol), (2S)-2,4-dimethylpiperazine-1-carbonyl chloride (43 mg, 0.248 mmol) and K₂CO₃ (68 mg, 0.497 mmol) in MeCN (5.0 mL) was stirred at 0° C. for 2 h. The reaction mixture was concentrated to dryness. The residue was purified by silica gel column with 5% MeOH in DCM to give the title compound, 100 mg, yield=74% as a light-yellow solid. LCMS m/z=543 [M+H]⁺.

### Preparation 139: 7-methoxy-4-(3-phenyl-1H-pyrazol-4-yl)quinazolin-6-yl(S)-2,4-dimethyl piperazine-1-carboxylate

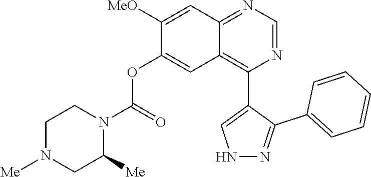 [SMILES: COc1cc2ncnc(-c3c[nH]nc3-c3ccccc3)c2cc1OC(=O)N1CCN(C)C[C@@H]1C]

To a solution of 7-methoxy-4-(3-phenyl-1-(tetrahydro-2H-pyran-2-yl)-1H-pyrazol-4-yl)quinazolin-6-yl (2S)-2,4-dimethylpiperazine-1-carboxylate (Preparation 138, 100 mg, 0.184 mmol) in DCM (5 mL), was added TFA (2 mL) at 0° C. and the reaction stirred at rt for 3 h. The mixture was concentrated to dryness, the residue was diluted with DCM and washed with sat. aq. Na₂CO₃. The organic layer was separated and dried over Na₂SO₄, filtered and concentrated to dryness to give the title compound, 100 mg, crude, as a light-yellow solid. LCMS m/z=459 [M+H]⁺.

### Preparation 140: 1-(tert-butyl) 4-(7-methoxy-4-(1-methyl-3-phenyl-1H-pyrazol-4-yl)quinazolin-6-yl)(2S,5S)-2,5-dimethylpiperazine-1,4-dicarboxylate

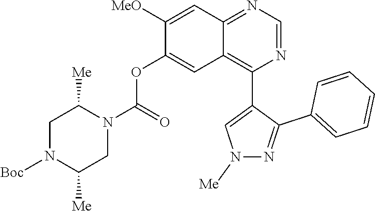 [SMILES: COc1cc2ncnc(-c3cn(C)nc3-c3ccccc3)c2cc1OC(=O)N1C[C@H](C)N(C(=O)OC(C)(C)C)C[C@@H]1C]

To a mixture of 7-methoxy-4-(1-methyl-3-phenyl-1H-pyrazol-4-yl)quinazolin-6-ol (Preparation 86, 100 mg, 0.301 mmol) and tert-butyl (2S,5S)-4-(chlorocarbonyl)-2,5-dimethylpiperazine-1-carboxylate (Preparation 58, 166.54 mg, 0.602 mmol) in DMF (2 mL) was added K₂CO₃ (83.17 mg, 0.602 mmol) and the reaction was stirred at 80° C. for 1 h. The mixture was filtered and the filtrate concentrated under reduced pressure to give the title compound (150 mg, crude) as yellow solid.

### Preparation 141: 4-(tert-butyl) 1-(7-methoxy-4-(1-methyl-3-phenyl-1H-pyrazol-4-yl)quinazolin-6-yl)(S)-2-methylpiperazine-1,4-dicarboxylate

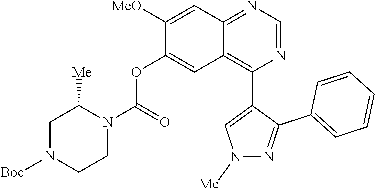 [SMILES: COc1cc2ncnc(-c3cn(C)nc3-c3ccccc3)c2cc1OC(=O)N1CCN(C(=O)OC(C)(C)C)C[C@@H]1C]

The title compound was obtained as a yellow solid, 100 mg, crude, from 7-methoxy-4-(1-methyl-3-phenyl-1H-pyrazol-4-yl)quinazolin-6-ol (Preparation 86) and tert-butyl (2S)-4-(chlorocarbonyl)-2,5-dimethylpiperazine-1-carboxylate, following the procedure described in Preparation 140.

### Preparation 142: 1-(tert-butyl) 4-(7-methoxy-4-(1-methyl-3-phenyl-1H-pyrazol-4-yl)quinazolin-6-yl) (2R,3S)-2,3-dimethylpiperazine-1,4-dicarboxylate

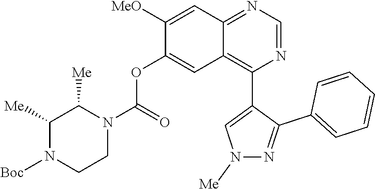 [SMILES: COc1cc2ncnc(-c3cn(C)nc3-c3ccccc3)c2cc1OC(=O)N1CCN(C(=O)OC(C)(C)C)[C@H](C)[C@@H]1C]

Part 1: To a solution of 7-methoxy-4-(1-methyl-3-phenyl-1H-pyrazol-4-yl)quinazolin-6-ol (Preparation 86, 500 mg, 1.50 mmol) and DIPEA (291.65 mg, 2.26 mmol) in THF (20 mL) was added triphosgene (1.050 g, 3.54 mmol) dropwise at 0° C. and the reaction was stirred at 25° C. for 1 h. The reaction mixture was concentrated under reduced pressure to give 7-methoxy-4-(1-methyl-3-phenyl-1H-pyrazol-4-yl)quinazolin-6-yl carbonochloridate (500 mg, crude) as a yellow solid which was used directly in the next step.

Part 2: To the solution of 7-methoxy-4-(1-methyl-3-phenyl-1H-pyrazol-4-yl)quinazolin-6-yl carbonochloridate (200 mg, 0.507 mmol) and (2R,3S)-tert-butyl 2,3-dimethylpiperazine-1-carboxylate (54.28 mg, 0.253 mmol) in DCM (2 mL) was added TEA (102.52 mg, 1.01 mmol) and the reaction was stirred at 25° C. for 2 h. The reaction mixture was concentrated under reduced pressure and the residue was purified by prep-HPLC (column: Waters Xbridge BEH C18 100*30 mm*10 um; mobile phase: [water (10 mM NH₄HCO₃)—MeCN]; B %: 30%-60%, 10 min) to give the title compound (80 mg, 27.6% yield) as a white solid. ¹H-NMR (400 MHZ, CDCl₃) δ ppm 9.18 (s, 1H), 7.79 (s, 1H), 7.45 (s, 1H), 7.40-7.34 (m, 3H), 7.23-7.18 (m, 3H), 6.80-6.73 (m, 1H), 4.06 (s, 3H), 4.05-4.01 (m, 1H), 3.98 (s, 3H), 3.97-3.92 (m, 1H), 3.72-3.63 (m, 2H), 3.57-3.44 (m, 2H), 1.50 (s, 9H), 1.36 (d, 3H), 1.29 (d, 3H).

### Preparation 143: 7-methoxy-4-(1-methyl-3-phenyl-1H-pyrazol-4-yl)quinazolin-6-yl (2S,5S)-2,5-dimethylpiperazine-1-carboxylate

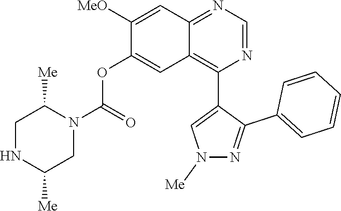 [SMILES: COc1cc2ncnc(-c3cn(C)nc3-c3ccccc3)c2cc1OC(=O)N1C[C@H](C)NC[C@@H]1C]

A mixture of 1-(tert-butyl) 4-(7-methoxy-4-(1-methyl-3-phenyl-1H-pyrazol-4-yl)quinazolin-6-yl)(2S,5S)-2,5-dimethylpiperazine-1,4-dicarboxylate (Preparation 140, 150 mg, 0.262 mmol) in HCl/EtOAc (5.0 mL) was stirred at 20° C. for 20 mins. The mixture was concentrated in vacuo and the residue purified by prep-HPLC (column: Phenomenex Gemini-NX C18 75*30 mm*3 um; mobile phase: [water (0.05% NH₃H₂O+10 mM NH₄HCO₃)—MeCN]; B %: 20%-45%, 8 min) to afford the title compound, 66.30 mg, 53.6% yield as pale yellow solid. ¹H-NMR (400 MHZ, CDCl₃) δ ppm 9.20 (s, 1H), 7.82 (s, 1H), 7.52 (s, 1H), 7.43-7.40 (m, 3H), 7.25-7.23 (m, 3H), 4.33-4.27 (m, 1H), 4.09 (s, 3H), 4.02 (s, 3H), 3.92-3.84 (m, 1H), 3.13-3.07 (m, 1H), 2.89 (br d, 1H, J=12.4 Hz), 2.84-2.67 (m, 2H), 1.35 (br dd, 3H, J=18.0, 6.8 Hz), 1.14-1.13 (m, 3H).

### Preparation 144: 7-methoxy-4-(1-methyl-3-phenyl-1H-pyrazol-4-yl)quinazolin-6-yl (2S,3R)-2,3-dimethylpiperazine-1-carboxylate

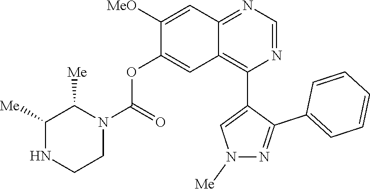 [SMILES: COc1cc2ncnc(-c3cn(C)nc3-c3ccccc3)c2cc1OC(=O)N1CCN[C@H](C)[C@@H]1C]

The title compound was obtained as a yellow solid, 36.2 mg, 58% yield, from 1-(tert-butyl) 4-(7-methoxy-4-(1-methyl-3-phenyl-1H-pyrazol-4-yl)quinazolin-6-yl)(2R,3S)-2,3-dimethylpiperazine-1,4-dicarboxylate (Preparation 142), following a similar procedure to that described in Preparation 143.

### Preparation 145: 7-methoxy-4-(1-methyl-3-phenyl-1H-pyrazol-4-yl)quinazolin-6-yl(S)-2-methylpiperazine-1-carboxylate trifluoroacetate

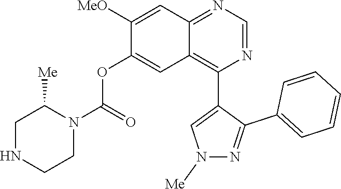 [SMILES: COc1cc2ncnc(-c3cn(C)nc3-c3ccccc3)c2cc1OC(=O)N1CCNC[C@@H]1C]

To a mixture of 4-(tert-butyl) 1-(7-methoxy-4-(1-methyl-3-phenyl-1H-pyrazol-4-yl)quinazolin-6-yl)(S)-2-methylpiperazine-1,4-dicarboxylate (Preparation 141, 100 mg, 0.179 mmol) in DCM (1.5 mL) was added TFA (0.5 mL) and the reaction stirred at 20° C. for 0.5 h. The mixture was concentrated under reduced pressure to give the title compound (120 mg, crude) as yellow oil.

### Preparation 146: methyl 5-(benzyloxy)-4-bromo-2-nitrobenzoate

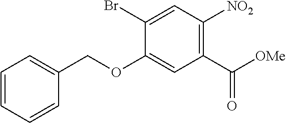 [SMILES: COC(=O)c1cc(OCc2ccccc2)c(Br)cc1[N+](=O)[O-]]

To a mixture of phenylmethanol (7.76 g, 71.8 mmol) in THF (200 mL) was added NaH (1.72 g, 71.8 mmol) at 0° C., the solution stirred at 0° C. for 15 min, then methyl 4-bromo-5-fluoro-2-nitrobenzoate (10 g, 35.9 mmol) was added and the reaction mixture was stirred at 25° C. for 16 h. The mixture was added to ice water and extracted with EtOAc. The organic phase was concentrated under vacuum and the residue was purified by column chromatography (50% EtOAc in PE) to give the title compound, 6 g, yield: 45.8% as a yellow solid. LCMS m/z=366 [M+H]⁺.

### Preparation 147: methyl 2-amino-5-(benzyloxy)-4-bromobenzoate

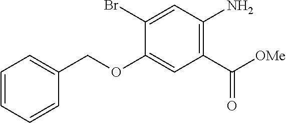 [SMILES: COC(=O)c1cc(OCc2ccccc2)c(Br)cc1N]

A mixture of methyl 5-(benzyloxy)-4-bromo-2-nitrobenzoate (Preparation 146, 6 g, 16.3 mmol), Fe (13.6 g, 244 mmol) and HCl (8.54 g, 244 mmol) in EtOH (100 mL) and water (10 mL) was stirred at 80° C. for 2 h. The cooled mixture was filtered and the filtrate was evaporated under reduced pressure. The crude solid was diluted with DCM (100 mL), basified with NaHCO₃ and extracted with DCM. The organic phase was dried over Na₂SO₄ and filtered and then concentrated to afford the title compound, 5 g, 91.4% yield as a yellow solid. LCMS m/z=336 [M+H]⁺.

### Preparation 148: 2-amino-5-(benzyloxy)-4-bromobenzoic Acid

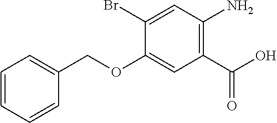 [SMILES: Nc1cc(Br)c(OCc2ccccc2)cc1C(=O)O]

To a solution of methyl 2-amino-5-(benzyloxy)-4-bromobenzoate (Preparation 147, 5 g, 14.8 mmol) in THF/H₂O was added LiOH (1.06 g, 44.4 mmol) and the reaction stirred at 25° C. for 4 h. The resulting solution was diluted with water (100 mL), extracted with EtOAc (2×50 mL) and the organic layers combined. The resulting solution was washed with brine (20 mL), dried over anhydrous Na₂SO₄ and evaporated under reduced pressure to give the title compound, 4 g, 84.0% as a yellow solid. LCMS m/z=322 [M+H]⁺.

### Preparation 149: 6-(benzyloxy)-7-bromoquinazolin-4(3H)-one

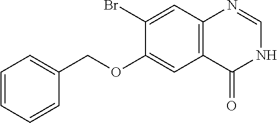 [SMILES: O=c1[nH]cnc2cc(Br)c(OCc3ccccc3)cc12]

To a solution of 2-amino-5-(benzyloxy)-4-bromobenzoic acid (Preparation 148, 4 g, 12.4 mmol) in EtOH (100 mL) was added ethanimidamide (3.60 g, 62.0 mmol) and the reaction was stirred for 16 h at 80° C. The solution was concentrated in vacuo, water added and the resulting solid filtered off to give the title compound as a yellow solid, 3.5 g, 85.3% as a yellow solid. LCMS m/z=322 [M+H]⁺.

### Preparation 150: 6-(benzyloxy)-7-bromo-4-chloroquinazoline

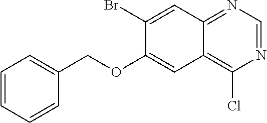 [SMILES: Clc1ncnc2cc(Br)c(OCc3ccccc3)cc12]

To a solution of 6-(benzyloxy)-7-bromo-3,4-dihydroquinazolin-4-one (Preparation 149, 3.5 g, 10.5 mmol) in MeCN (100 mL) was added TEA (6.36 g, 63.0 mmol), then phosphoroyl trichloride (8.04 g, 52.5 mmol) and the reaction was stirred at 80° C. for 3 h. The cooled mixture was poured into sodium bicarbonate solution and extracted with EtOAc. The organic layer was dried (MgSO₄), filtered and the solvent was evaporated to dryness. The crude product was purified by column chromatography on silica gel (PE:EtOAc=2:1) to give the title compound, 2.0 g, 54.4% as a yellow solid. LCMS m/z=350 [M+H]⁺.

### Preparation 151: 6-(benzyloxy)-7-bromo-4-(1-methyl-3-phenyl-1H-pyrazol-4-yl)quinazoline

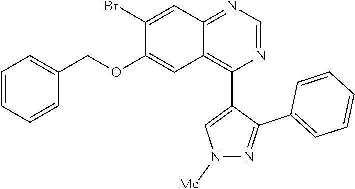 [SMILES: Cn1cc(-c2ncnc3cc(Br)c(OCc4ccccc4)cc23)c(-c2ccccc2)n1]

A mixture of 6-(benzyloxy)-7-bromo-4-chloroquinazoline (Preparation 150, 699 mg, 2 mmol), 1-methyl-3-phenyl-4-(4,4,5,5-tetramethyl-1,3,2-dioxaborolan-2-yl)-1H-pyrazole (625 mg, 2.20 mmol), Pd(PPh₃)₄ (346 mg, 0.30 mmol) and K₃PO₄ (805 mg, 3.80 mmol) in DMSO (20 mL) was heated to 80° C. for 3 h. After cooling down to rt, the mixture was quenched with 5% NaOH(aq)(50 mL). The mixture was extracted with EtOAc (20 mL×3), the organic layers combined and concentrated in vacuo. The residue was purified by silica gel column chromatography (DCM/EtOAc=1:1 to DCM/MeOH=15:1) to give the title compound, 300 mg as a yellow solid.

### Preparation 152: 7-bromo-4-(1-methyl-3-phenyl-1H-pyrazol-4-yl)quinazolin-6-ol

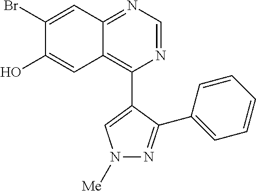 [SMILES: Cn1cc(-c2ncnc3cc(Br)c(O)cc23)c(-c2ccccc2)n1]

A solution of 6-(benzyloxy)-7-bromo-4-(1-methyl-3-phenyl-1H-pyrazol-4-yl)quinazoline (Preparation 151, 282 mg, 0.6 mmol) in TFA (20 mL) was heated to 80° C. for 3 h. After cooling down to rt, the mixture was evaporated under reduced pressure to afford the title compound (190 mg, 83%) as a yellow syrup.

### Preparation 153: 7-bromo-4-(1-methyl-3-phenyl-1H-pyrazol-4-yl)quinazolin-6-yl(S)-2,4-dimethyl piperazine-1-carboxylate

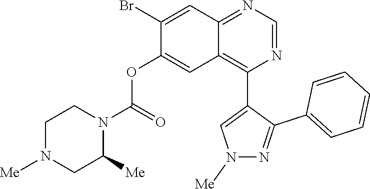 [SMILES: C[C@H]1CN(C)CCN1C(=O)Oc1cc2c(-c3cn(C)nc3-c3ccccc3)ncnc2cc1Br]

A mixture of 7-bromo-4-(1-methyl-3-phenyl-1H-pyrazol-4-yl)quinazolin-6-ol (Preparation 152, 190 mg, 0.5 mmol), (2S)-2,4-dimethylpiperazine-1-carbonyl chloride (176 mg, 1 mmol) and K₂CO₃ (207 mg, 1.5 mmol) in MeCN (5 mL) was heated to 80° C. for 3 h. The cooled mixture was concentrated in vacuo and the residue was purified by prep-TLC (DCM/MeOH=15:1) to afford the title compound (200 mg, 76.9%) as a yellow syrup. LCMS m/z=521 [M+H]⁺.

### Preparation 154: (S)-6-bromo-7-((tetrahydrofuran-3-yl)oxy) quinazolin-4 (3H)-one

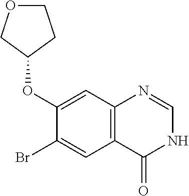 [SMILES: O=c1[nH]cnc2cc(O[C@H]3CCOC3)c(Br)cc12]

To a solution of 6-bromo-7-fluoroquinazolin-4 (3H)-one (200 mg, 0.826 mmol) and (S)-tetrahydrofuran-3-ol (145 mg, 1.65 mmol) in DMF (10 mL), was added NaH (60%, 66 mg, 1.65 mmol) at rt and the reaction stirred at 80° C. for 3 h. The reaction mixture was quenched with water and extracted with DCM. The combined organic layers were dried over Na₂SO₄, filtered and concentrated to dryness. The residue was purified by prep-TLC with DCM:MeOH=25:1 to afford the title compound (180 mg, yield: 70%) as a white solid. LCMS: m/z=311, 313 [M+H]⁺.

### Preparation 155: (S)-6-bromo-4-chloro-7-((tetrahydrofuran-3-yl)oxy)quinazoline

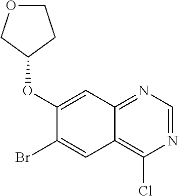 [SMILES: Clc1ncnc2cc(O[C@H]3CCOC3)c(Br)cc12]

A solution of(S)-6-bromo-7-((tetrahydrofuran-3-yl)oxy) quinazolin-4 (3H)-one (Preparation 154, 180 mg, 0.579 mmol) in POCl₃ (5 mL) was stirred at 100° C. for 3 h. The cooled reaction was concentrated to dryness and the residue was then diluted with DCM (5 mL). The resulting solution was diluted with sat. Na₂CO₃ (aq)(30 mL) and extracted with DCM. The organic layer was dried over Na₂SO₄, filtered and concentrated to dryness to afford the title compound (200 mg, crude) as a brown solid. LCMS: m/z=330 [M+H]⁺

### Preparation 156: (S)-6-bromo-4-(1-(2,2-difluoroethyl)-3-phenyl-1H-pyrazol-4-yl)-7-((tetrahydrofuran-3-yl)oxy)quinazoline

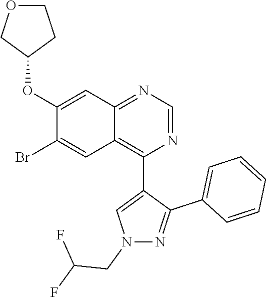 [SMILES: FC(F)Cn1cc(-c2ncnc3cc(O[C@H]4CCOC4)c(Br)cc23)c(-c2ccccc2)n1]

The title compound was obtained as a yellow solid, 150 mg, 50% yield, from (S)-6-bromo-4-chloro-7-((tetrahydrofuran-3-yl)oxy)quinazoline (Preparation 155) and 1-(2,2-difluoroethyl)-3-phenyl-4-(4,4,5,5-tetramethyl-1,3,2-dioxaborolan-2-yl)-1H-pyrazole (Preparation 41) following the procedure described in Preparation 90. LCMS: m/z=501, 503 [M+H]⁺.

### Preparation 157: 6-bromo-4-(1-(2,2-difluoroethyl)-3-phenyl-1H-pyrazol-4-yl)-7-methoxy quinazoline

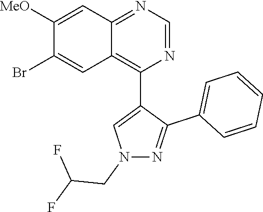 [SMILES: COc1cc2ncnc(-c3cn(CC(F)F)nc3-c3ccccc3)c2cc1Br]

The title compound was obtained, 400 mg, 90% yield, from 6-bromo-4-chloro-7-methoxyquinazoline and 1-(2,2-difluoroethyl)-3-phenyl-4-(4,4,5,5-tetramethyl-1,3,2-dioxaborolan-2-yl)-1H-pyrazole (Preparation 41) following a similar procedure to that described in Preparation 90. LCMS: m/z=445, 447 [M+H]⁺.

### Preparation 158: 6-bromo-4-(1-ethyl-3-(4-fluorophenyl)-1H-pyrazol-4-yl)-7-methoxyquinazoline

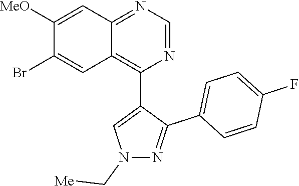 [SMILES: CCn1cc(-c2ncnc3cc(OC)c(Br)cc23)c(-c2ccc(F)cc2)n1]

The title compound was obtained as a light yellow solid, 50 mg, 37% yield, from 1-ethyl-3-(4-fluorophenyl)-4-(4,4,5,5-tetramethyl-1,3,2-dioxaborolan-2-yl)-1H-pyrazole (Preparation 44) and 6-bromo-4-chloro-7-methoxyquinazoline, following a similar procedure to that described in Preparation 90. LCMS m/z=427 [M+H]⁺.

### Preparation 159: 6-bromo-4-(1-(2,2-difluoroethyl)-3-phenyl-1H-pyrazol-4-yl)-7-ethoxyquinazoline

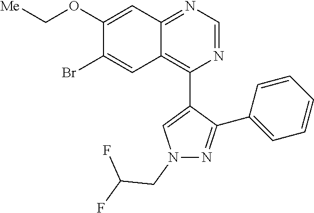 [SMILES: CCOc1cc2ncnc(-c3cn(CC(F)F)nc3-c3ccccc3)c2cc1Br]

Pd(dppf)Cl₂ (76.0 mg, 0.104 mmol) and K₂CO₃ (215 mg, 1.56 mmol) were added to 6-bromo-4-chloro-7-ethoxyquinazoline (Preparation 89, 300 mg, 1.04 mmol) and 1-(2,2-difluoroethyl)-3-phenyl-4-(4,4,5,5-tetramethyl-1,3,2-dioxaborolan-2-yl)-1H-pyrazole (Preparation 41, 380 mg, 1.14 mmol) in dioxane/H₂O (10 mL/2.5 mL) and the reaction mixture was heated at 80° C. for 16 h. The reaction mixture was diluted with EtOAc (100 mL) and washed sequentially with water (100 mL×3) and brine (100 mL). The organic layer was dried over Na₂SO₄, filtered and evaporated under reduced pressure. The crude product was purified by prep-TLC eluting with PE:EtOAc=1:1 to give the title compound, 360 mg (75.4%) as a light-yellow solid. LCMS m/z=458 [M+H]⁺.

### Preparation 160: 6-bromo-7-ethoxy-4-(3-phenyl-1H-pyrazol-4-yl)quinazoline

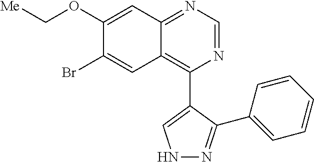 [SMILES: CCOc1cc2ncnc(-c3c[nH]nc3-c3ccccc3)c2cc1Br]

To a solution of 6-bromo-7-ethoxy-4-(3-phenyl-1-(tetrahydro-2H-pyran-2-yl)-1H-pyrazol-4-yl)quinazoline (Preparation 95, 600 mg, 1.25 mmol) in DCM (9 mL) was added TFA (3 mL) and the reaction stirred at rt for 1 h. The reaction was concentrated to dryness, the residue was diluted with sat. aq. Na₂CO₃ and extracted with DCM. The combined organic layer was dried over Na₂SO₄, filtered and concentrated to dryness to afford the title compound, 400 mg, yield=80% as a light yellow solid. LCMS: m/z=395, 397 [M+H]⁺.

### Preparation 161: 6-bromo-7-ethoxy-4-(1-isopropyl-3-phenyl-1H-pyrazol-4-yl)quinazoline

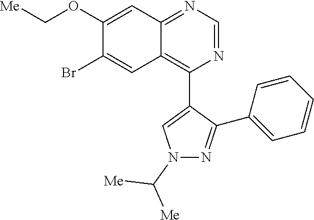 [SMILES: CCOc1cc2ncnc(-c3cn(C(C)C)nc3-c3ccccc3)c2cc1Br]

To a mixture of 6-bromo-7-ethoxy-4-(3-phenyl-1H-pyrazol-4-yl)quinazoline (Preparation 160, 200 mg, 0.505 mmol) and 2-iodopropane (171 mg, 1.01 mmol) in DMF (10 mL) was added NaH (24.2 mg, 1.01 mmol) and the reaction stirred at 50° C. for 1 h. The reaction was quenched with ice-water then concentrated to dryness. The residue was purified by prep-TLC with DCM:MeOH=20:1 to afford the title compound (150 mg, yield=68%) as a light-yellow solid. LCMS: m/z=437, 439 [M+H]⁺.

### Preparation 162: 6-bromo-4-(1-cyclopropyl-3-phenyl-1H-pyrazol-4-yl)-7-ethoxyquinazoline

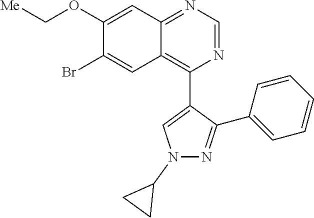 [SMILES: CCOc1cc2ncnc(-c3cn(C4CC4)nc3-c3ccccc3)c2cc1Br]

A mixture of Cu(OAc)₂ (92 mg, 0.760 mmol) and bipyridine (118 mg, 0.760 mmol) in DCE (15 mL) was stirred at 80° C. for 30 min, then allowed to cool. 6-Bromo-7-ethoxy-4-(3-phenyl-1H-pyrazol-4-yl)quinazoline (Preparation 160, 150 mg, 0.380 mmol), cyclopropylboronic acid (65 mg, 0.760 mmol) and Na₂CO₃ (80 mg, 0.760 mmol) were added and the reaction stirred at 80° C. under an atmosphere of O₂ for 4 h. The mixture was concentrated to dryness and the residue was purified by prep-TLC with DCM:MeOH=25:1 to afford the title compound (80 mg, yield: 48%) as a light-yellow solid. LCMS: m/z=435, 437 [M+H]⁺.

### Preparation 163: 6-(1-ethoxyvinyl)-7-methoxy-4-(1-methyl-3-phenyl-1H-pyrazol-4-yl)quinazoline

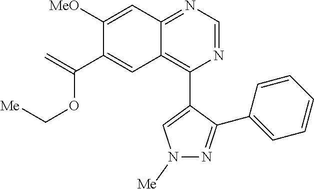 [SMILES: C=C(OCC)c1cc2c(-c3cn(C)nc3-c3ccccc3)ncnc2cc1OC]

To a solution of 7-methoxy-4-(1-methyl-3-phenyl-1H-pyrazol-4-yl)quinazolin-6-yl trifluoromethanesulfonate (Preparation 116, 1 g, 2.15 mmol) in dioxane/H₂O (20 mL/5 mL) was added tributyl(1-ethoxyethenyl) stannane (776 mg, 2.15 mmol), K₂CO₃ (593 mg, 4.30 mmol) and Pd(PPh₃)₂Cl₂ (171 mg, 0.21 mmol) under N₂ and the reaction was stirred at 100° C. for 4 h. The reaction mixture was cooled to rt, then diluted with water (25 mL). The resulting solution was extracted with EtOAc (2×40 mL) and the organic layers combined. The resulting mixture was washed with brine (20 mL), dried over anhydrous Na₂SO₄ and concentrated under vacuum. The product was purified by chromatography with PE:EtOAc (1:1) to give the title compound, 600 mg (72.2%), as a yellow solid. LCMS m/z=387 [M+H]⁺.

### Preparation 164: 4-(1-(2,2-difluoroethyl)-3-phenyl-1H-pyrazol-4-yl)-6-(1-ethoxyvinyl)-7-methoxy quinazoline

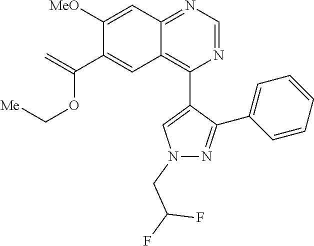 [SMILES: C=C(OCC)c1cc2c(-c3cn(CC(F)F)nc3-c3ccccc3)ncnc2cc1OC]

The title compound was obtained as a white solid, 211 mg, 48%, from 4-(1-(2,2-difluoroethyl)-3-phenyl-1H-pyrazol-4-yl)-7-methoxyquinazolin-6-yl trifluoromethanesulfonate (Preparation 117) following the procedure described in Preparation 163. LCMS m/z=437 [M+H]⁺.

### Preparation 165: 7-ethoxy-6-(1-ethoxyvinyl)-4-(1-methyl-3-phenyl-1H-pyrazol-4-yl)quinazoline

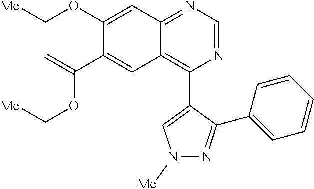 [SMILES: C=C(OCC)c1cc2c(-c3cn(C)nc3-c3ccccc3)ncnc2cc1OCC]

A mixture of 6-bromo-7-ethoxy-4-(1-methyl-3-phenyl-1H-pyrazol-4-yl)quinazoline (Preparation 90, 200 mg, 0.488 mmol), (1-ethoxyethenyl)triethylstannane (270 mg, 0.976 mmol), Pd(PPh₃)₂Cl₂ (30 mg, 0.043 mmol) and Cs₂CO₃ (317 mg, 0.976 mmol) in dioxane (15 mL) was stirred at 100° C. for 4 h and the reaction mixture then concentrated in vacuum. The residue was purified by prep-TLC with DCM:MeOH=15:1 to afford the title compound (160 mg, yield: 75.8%) as a grey-yellow solid. LCMS: m/z=401 [M+H]⁺.

### Preparation 166: 7-ethoxy-6-(1-ethoxyvinyl)-4-(1-ethyl-3-phenyl-1H-pyrazol-4-yl)quinazoline

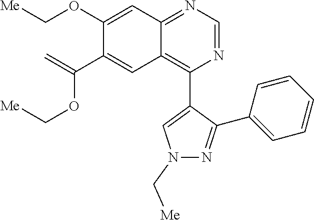 [SMILES: C=C(OCC)c1cc2c(-c3cn(CC)nc3-c3ccccc3)ncnc2cc1OCC]

A mixture of 6-bromo-7-ethoxy-4-(1-ethyl-3-phenyl-1H-pyrazol-4-yl)quinazoline (Preparation 93, 180 mg, 0.425 mmol), tributyl(1-ethoxyethenyl) stannane (230 mg, 0.637 mmol) and Pd(PPh₃)₂Cl₂ (29.8 mg, 0.0425 mmol) in dioxane (15 mL) was stirred at 100° C. for 2 h and concentrated to dryness. The residue was purified by prep-TLC with DCM:MeOH=20:1 to afford the title compound (130 mg, 74%) as a light-yellow solid. LCMS: m/z=415 [M+H]⁺.

### Preparation 167 to 171

The compounds in the following table were prepared from the appropriate 6-bromoquinazoline and tributyl(1-ethoxyethenyl) stannane, following a similar procedure to that described in Preparation 166.

| Preparation | |
|---|---|
| No | Name, Structure, Starting material (SM), Data |
| 167 | 6-(1-ethoxyvinyl)-4-(1-ethyl-3-phenyl-1H-pyrazol-4-yl)-7-methoxyquinazoline |
| | 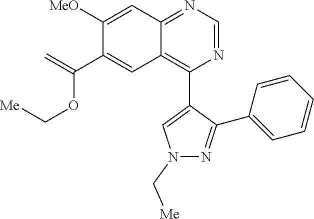 [SMILES: C=C(OCC)c1cc2c(-c3cn(CC)nc3-c3ccccc3)ncnc2cc1OC] |
| | SM: 6-bromo-4-(1-ethyl-3-phenyl-1H-pyrazol-4-yl)-7-methoxyquinazoline |
| | (Preparation 94). |
| | yellow solid, 240 mg, 90% yield. LCMS m/z = 401 [M + H]⁺ |
| 168 | (S)-4-(1-(2,2-difluoroethyl)-3-phenyl-1H-pyrazol-4-yl)-6-(1-ethoxyvinyl)-7- |
| | ((tetrahydrofuran-3-yl)oxy)quinazoline |
| | 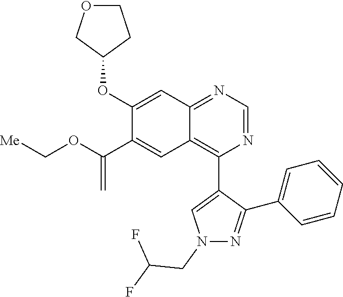 [SMILES: C=C(OCC)c1cc2c(-c3cn(CC(F)F)nc3-c3ccccc3)ncnc2cc1O[C@H]1CCOC1] |
| | SM: (S)-6-bromo-4-(1-(2,2-difluoroethyl)-3-phenyl-1H-pyrazol-4-yl)-7- |
| | ((tetrahydrofuran-3-yl)oxy)quinazoline (Preparation 156) |
| | 110 mg, yield = 75% as a light-yellow solid. LCMS: m/z = 493 [M + H]⁺. |
| 169 | 7-ethoxy-6-(1-ethoxyvinyl)-4-(1-isopropyl-3-phenyl-1H-pyrazol-4-yl)quinazoline |
| | 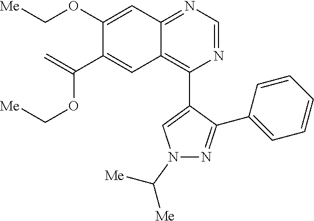 [SMILES: C=C(OCC)c1cc2c(-c3cn(C(C)C)nc3-c3ccccc3)ncnc2cc1OCC] |
| | SM: 6-bromo-7-ethoxy-4-(1-isopropyl-3-phenyl-1H-pyrazol-4-yl)quinazoline |
| | (Preparation 161) |
| | 120 mg, 55% yield. LCMS m/z = 429 [M + H]⁺ |
| 170 | 4-(1-cyclopropyl-3-phenyl-1H-pyrazol-4-yl)-7-ethoxy-6-(1- |
| | ethoxyvinyl)quinazoline |
| | 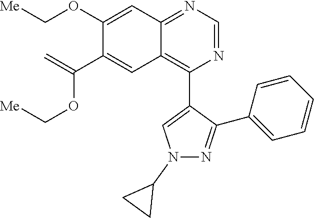 [SMILES: C=C(OCC)c1cc2c(-c3cn(C4CC4)nc3-c3ccccc3)ncnc2cc1OCC] |
| | SM: 6-bromo-4-(1-cyclopropyl-3-phenyl-1H-pyrazol-4-yl)-7-ethoxyquinazoline |
| | (Preparation 162) |
| | yellow solid, 125 mg, 85%. LCMS m/z = 427 [M + H]⁺ |
| 171 | 6-(1-ethoxyvinyl)-7-isopropoxy-4-(1-methyl-3-phenyl-1H-pyrazol-4- |
| | yl)quinazoline |
| | 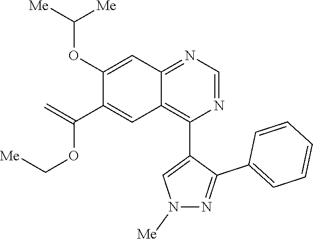 [SMILES: C=C(OCC)c1cc2c(-c3cn(C)nc3-c3ccccc3)ncnc2cc1OC(C)C] |
| | SM: 6-bromo-7-isopropoxy-4-(1-methyl-3-phenyl-1H-pyrazol-4-yl)quinazoline |
| | (Preparation 102) |
| | light yellow solid, 300 mg, 85% yield. LCMS m/z = 415 [M + H]⁺ |

### Preparation 172: 1-(7-methoxy-4-(1-methyl-3-phenyl-1H-pyrazol-4-yl)quinazolin-6-yl)ethan-1-one

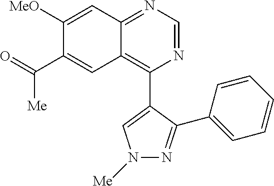 [SMILES: COc1cc2ncnc(-c3cn(C)nc3-c3ccccc3)c2cc1C(C)=O]

A mixture of 6-(1-ethoxyvinyl)-7-methoxy-4-(1-methyl-3-phenyl-1H-pyrazol-4-yl)quinazoline (Preparation 163, 150 mg, 0.388 mmol) in TFA (3.0 mL) and DCM (3.0 mL) was stirred at 25° C. for 1 h. The mixture was evaporated under reduced pressure to give the title compound as a yellow solid, 120 mg, 86.3%. LCMS m/z=359 [M+H]⁺.

### Preparation 173: 1-(4-(1-(2,2-difluoroethyl)-3-phenyl-1H-pyrazol-4-yl)-7-methoxyquinazolin-6-yl)ethan-1-one

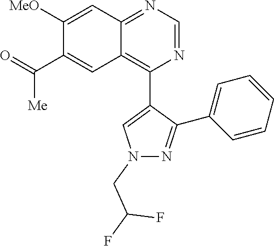 [SMILES: COc1cc2ncnc(-c3cn(CC(F)F)nc3-c3ccccc3)c2cc1C(C)=O]

1-(4-(1-(2,2-Difluoroethyl)-3-phenyl-1H-pyrazol-4-yl)-7-methoxyquinazolin-6-yl)ethan-1-one was obtained from 4-(1-(2,2-difluoroethyl)-3-phenyl-1H-pyrazol-4-yl)-6-(1-ethoxyvinyl)-7-methoxyquinazoline (Preparation 164), following the procedure described in Preparation 172. LCMS m/z=409 [M+H]⁺.

### Preparation 174: 1-(7-isopropoxy-4-(1-methyl-3-phenyl-1H-pyrazol-4-yl)quinazolin-6-yl)ethan-1-one

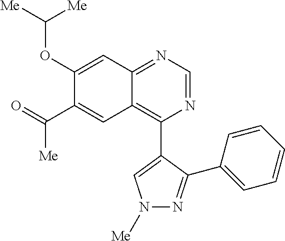 [SMILES: CC(=O)c1cc2c(-c3cn(C)nc3-c3ccccc3)ncnc2cc1OC(C)C]

The title compound was obtained as a light yellow solid, from 6-(1-ethoxyvinyl)-7-isopropoxy-4-(1-methyl-3-phenyl-1H-pyrazol-4-yl)quinazoline (Preparation 171), following the procedure described in Preparation 172. LCMS m/z=387 [M+H]⁺.

### Preparation 175: (S)-1-(4-(1-(2,2-difluoroethyl)-3-phenyl-1H-pyrazol-4-yl)-7-((tetrahydrofuran-3-yl)oxy) quinazolin-6-yl)ethan-1-one

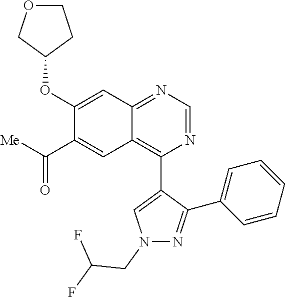 [SMILES: CC(=O)c1cc2c(-c3cn(CC(F)F)nc3-c3ccccc3)ncnc2cc1O[C@H]1CCOC1]

A mixture of(S)-4-(1-(2,2-difluoroethyl)-3-phenyl-1H-pyrazol-4-yl)-6-(1-ethoxyvinyl)-7-((tetrahydrofuran-3-yl)oxy)quinazoline (Preparation 168, 110 mg, 0.223 mmol) in DCM (12 mL) and TFA (4 mL) was stirred at rt for 1 h then concentrated to dryness. The residue was purified by prep-TLC with DCM:MeOH=30:1 to afford the title compound (90 mg, yield=86%) as a light-yellow solid. LCMS: m/z=465 [M+H]⁺.

### Preparation 176: 1-(4-(1-cyclopropyl-3-phenyl-1H-pyrazol-4-yl)-7-ethoxyquinazolin-6-yl)ethan-1-one

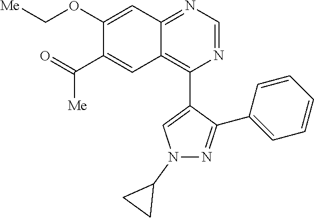 [SMILES: CCOc1cc2ncnc(-c3cn(C4CC4)nc3-c3ccccc3)c2cc1C(C)=O]

The title compound was obtained as an off-white solid, 95 mg, 81% yield, from 4-(1-cyclopropyl-3-phenyl-1H-pyrazol-4-yl)-7-ethoxy-6-(1-ethoxyvinyl)quinazoline (Preparation 170), following the procedure described in Preparation 175. LCMS m/z=399 [M+H]⁺.

### Preparation 177: 1-(7-ethoxy-4-(1-methyl-3-phenyl-1H-pyrazol-4-yl)quinazolin-6-yl)ethan-1-one

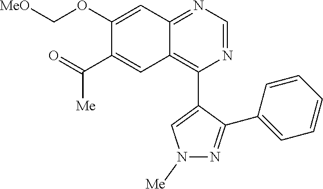 [SMILES: COCOc1cc2ncnc(-c3cn(C)nc3-c3ccccc3)c2cc1C(C)=O]

A mixture of 7-ethoxy-6-(1-ethoxyvinyl)-4-(1-methyl-3-phenyl-1H-pyrazol-4-yl)quinazoline (Preparation 165, 260 mg, 0.601 mmol) in TFA (2 mL) and DCM (10 mL) was stirred at 25° C. for 2 h and the mixture concentrated in vacuo. The residue was diluted with sat. aq. Na₂CO₃ and extracted with EtOAc. The combined organic layers were dried over Na₂SO₄, filtered and concentrated to dryness to afford the title compound (240 mg, yield: 99.1%) as a yellow solid. LCMS m/z=373 [M+H]⁺.

### Preparation 178: 1-(7-ethoxy-4-(1-ethyl-3-phenyl-1H-pyrazol-4-yl)quinazolin-6-yl)ethan-1-one

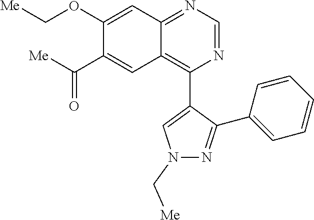 [SMILES: CCOc1cc2ncnc(-c3cn(CC)nc3-c3ccccc3)c2cc1C(C)=O]

The title compound was obtained as an off-white solid, 100 mg 82% yield from 7-ethoxy-6-(1-ethoxyvinyl)-4-(1-ethyl-3-phenyl-1H-pyrazol-4-yl)quinazoline (Preparation 166), following the procedure described in Preparation 177. LCMS: m/z=387 [M+H]⁺.

### Preparation 179: 1-(4-(1-ethyl-3-phenyl-1H-pyrazol-4-yl)-7-methoxyquinazolin-6-yl)ethan-1-one

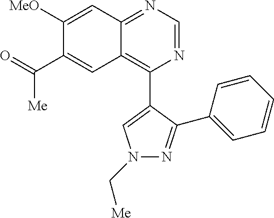 [SMILES: CCn1cc(-c2ncnc3cc(OC)c(C(C)=O)cc23)c(-c2ccccc2)n1]

The title compound was obtained as a yellow solid, 140 mg, 68% yield, from 6-(1-ethoxyvinyl)-4-(1-ethyl-3-phenyl-1H-pyrazol-4-yl)-7-methoxyquinazoline (Preparation 167), following the procedure described in Preparation 177. LCMS m/z=373 [M+H]⁺.

### Preparation 180: 1-(7-ethoxy-4-(1-isopropyl-3-phenyl-1H-pyrazol-4-yl)quinazolin-6-yl)ethan-1-one

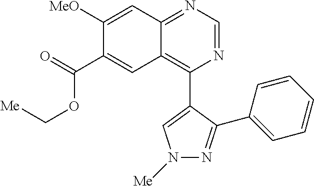 [SMILES: CCOC(=O)c1cc2c(-c3cn(C)nc3-c3ccccc3)ncnc2cc1OC]

A solution of 7-ethoxy-6-(1-ethoxyvinyl)-4-(1-isopropyl-3-phenyl-1H-pyrazol-4-yl)quinazoline (Preparation 169, 120 mg, 0.238 mmol) in DCM (10 mL) and TFA (2 mL) was stirred at 25° C. for 2 h and then concentrated to dryness. The residue was diluted with sat. aq. Na₂CO₃ and extracted with EtOAc. The organic layer was dried over Na₂SO₄, filtered and concentrated to dryness and purified on prep-TLC with DCM:MeOH=40:1 to afford the title compound (76 mg, yield: 57.3%) as a yellow solid. LCMS: m/z=401 [M+H]⁺.

### Preparation 181: ethyl 7-methoxy-4-(1-methyl-3-phenyl-1H-pyrazol-4-yl)quinazoline-6-carboxylate

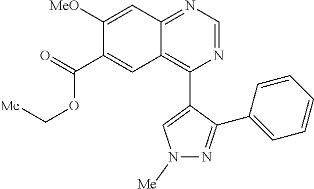 [SMILES: CCOC(=O)c1cc2c(-c3cn(C)nc3-c3ccccc3)ncnc2cc1OC]

To a solution of 6-(1-ethoxyvinyl)-7-methoxy-4-(1-methyl-3-phenyl-1H-pyrazol-4-yl)quinazoline (Preparation 163, 150 mg, 0.388 mmol) in dioxane (6 mL), was added a solution of NaIO₄ (166 mg, 0.776 mmol) in water (2 mL), followed by the batchwise addition of KMnO₄ (12.2 mg, 0.078 mmol). The resulting solution was stirred for 2 h at 25° C. then extracted with DCM (2×20 mL) and the organic layers combined. The resulting mixture was washed with H₂O (2×20 mL) and brine (10 mL), dried over Na₂SO₄ and concentrated under vacuum. The residue was purified by TLC DCM:MeOH=10:1 to give the title compound, 100 mg (66.6%) as a white solid. LCMS m/z=389 [M+H]⁺.

### Preparation 182: ethyl 4-(1-(2,2-difluoroethyl)-3-phenyl-1H-pyrazol-4-yl)-7-methoxyquinazoline-6-carboxylate

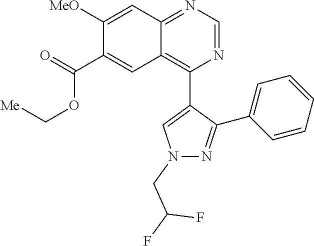 [SMILES: CCOC(=O)c1cc2c(-c3cn(CC(F)F)nc3-c3ccccc3)ncnc2cc1OC]

Ethyl 4-(1-(2,2-difluoroethyl)-3-phenyl-1H-pyrazol-4-yl)-7-methoxyquinazoline-6-carboxylate was obtained as a white solid, 100 mg, 66.6% yield, from 4-(1-(2,2-difluoroethyl)-3-phenyl-1H-pyrazol-4-yl)-6-(1-ethoxyvinyl)-7-methoxyquinazoline (Preparation 164), following a similar procedure to that described in Preparation 181. LCMS m/z=439 [M+H]⁺.

### Preparation 183: 4-(1-(2,2-difluoroethyl)-3-phenyl-1H-pyrazol-4-yl)-7-methoxy-6-(1-(4,4,5,5-tetramethyl-1,3,2-dioxaborolan-2-yl)cyclopropyl)quinazoline

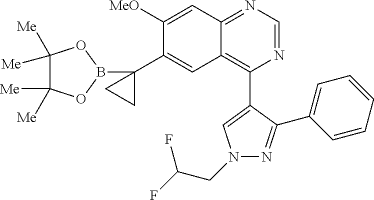 [SMILES: COc1cc2ncnc(-c3cn(CC(F)F)nc3-c3ccccc3)c2cc1C1(B2OC(C)(C)C(C)(C)O2)CC1]

Cs₂CO₃ (5.11 g, 15.7 mmol) was added to cataCXium A Pd G3 (389 mg, 0.524 mmol), 4-(1-(2,2-difluoroethyl)-3-phenyl-1H-pyrazol-4-yl)-7-methoxyquinazolin-6-yl trifluoromethanesulfonate (Preparation 117, 2.7 g, 5.24 mmol) and 2,2′-(cyclopropane-1,1-diyl)bis(4,4,5,5-tetramethyl-1,3,2-dioxaborolane) (4.61 g, 15.7 mmol) in dioxane/H₂O (50 mL/5 mL) and the reaction was stirred at 100° C. for 16 h. The mixture was diluted with EtOAc (300 mL), washed with brine (100 mL×2), the organic layer was dried over Na₂SO₄ and concentrated under vacuum. The residue was purified by silica gel column eluting with DCM:MeOH=25:1 to afford the title compound, 2.4 g, as a yellow solid. LCMS: m/z=533 [M+H]⁺.

### Preparation 184: tert-butyl 6-(7-methoxy-4-(1-methyl-3-phenyl-1H-pyrazol-4-yl)quinazolin-6-yl)-2,3-dihydro-4H-1,4-oxazine-4-carboxylate

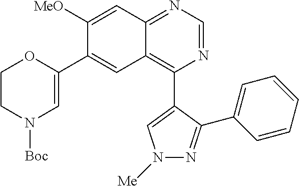 [SMILES: COc1cc2ncnc(-c3cn(C)nc3-c3ccccc3)c2cc1C1=CN(C(=O)OC(C)(C)C)CCO1]

Pd(dppf)Cl₂ (36.9 mg, 0.051 mmol) and K₂CO₃ (104 mg, 0.757 mmol) were added to 6-bromo-7-methoxy-4-(1-methyl-3-phenyl-1H-pyrazol-4-yl)quinazoline (Preparation 91, 200 mg, 0.505 mmol) and tert-butyl 6-(4,4,5,5-tetramethyl-1,3,2-dioxaborolan-2-yl)-2,3-dihydro-4H-1,4-oxazine-4-carboxylate (188 mg, 0.606 mmol) in H₂O (1 mL) and dioxane (4 mL) at rt and the reaction mixture was heated at 80° C. for 2 h under N₂. The resulting solution was extracted with EtOAc (3×50 mL), the combined organic layer was dried over (Na₂SO₄) and concentrated under vacuum. The crude product was purified by prep-TLC with DCM:MeOH=25:1 to give the title compound, 150 mg (59%) as a yellow solid. LCMS: m/z=500 [M+H]⁺.

### Preparation 185: 6-(7-methoxy-4-(1-methyl-3-phenyl-1H-pyrazol-4-yl)quinazolin-6-yl)-3,4-dihydro-2H-1,4-oxazine trifluoroacetate

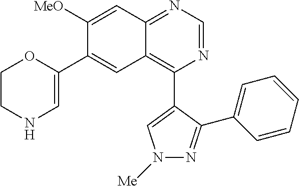 [SMILES: COc1cc2ncnc(-c3cn(C)nc3-c3ccccc3)c2cc1C1=CNCCO1]

TFA (2 mL) was added to tert-butyl 6-(7-methoxy-4-(1-methyl-3-phenyl-1H-pyrazol-4-yl)quinazolin-6-yl)-2,3-dihydro-4H-1,4-oxazine-4-carboxylate (Preparation 184, 60 mg, 0.12 mmol) in DCM (5 mL) and the reaction mixture was stirred at rt for 2 h. The mixture was concentrated under vacuum to afford the title compound, 40 mg (83%) as a yellow oil. LCMS: m/z=400 [M+H]⁺.

### Preparation 186: 6-(2,5-dihydro-1H-pyrrol-3-yl)-7-methoxy-4-(1-methyl-3-phenyl-1H-pyrazol-4-yl)quinazoline trifluoroacetate

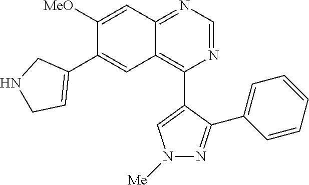 [SMILES: COc1cc2ncnc(-c3cn(C)nc3-c3ccccc3)c2cc1C1=CCNC1]

A mixture of 6-bromo-7-methoxy-4-(1-methyl-3-phenyl-1H-pyrazol-4-yl)quinazoline (Preparation 91, 50 mg, 0.126 mmol), Pd(PPh₃)₄ (14.62 mg, 0.013 mmol), THF (0.5 mL), K₃PO₄ (126 μl, 0.253 mmol) and tert-butyl 3-(4,4,5,5-tetramethyl-1,3,2-dioxaborolan-2-yl)-2,5-dihydro-1H-pyrrole-1-carboxylate (56 mg, 0.19 mmol) was stirred at 80° C. The cooled mixture was diluted with water and DCM, the layers separated and the organic phase concentrated in vacuo. The residue was purified by silica gel column (MeOH:DCM) to provide tert-butyl 3-(7-methoxy-4-(1-methyl-3-phenyl-1H-pyrazol-4-yl)quinazolin-6-yl)-2,5-dihydro-1H-pyrrole-1-carboxylate. This was dissolved in TFA (1 mL) and the solution stirred at rt overnight. The solution was evaporated under reduced pressure to afford the title compound.

### Preparation 187: (7-ethoxy-4-(1-methyl-3-phenyl-1H-pyrazol-4-yl)quinazolin-6-yl)methyl methanesulfonate

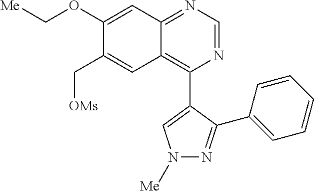 [SMILES: CCOc1cc2ncnc(-c3cn(C)nc3-c3ccccc3)c2cc1COS(C)(=O)=O]

Methanesulfonyl chloride (155 mg, 1.25 mmol) was added dropwise to an ice-cooled solution of (7-ethoxy-4-(1-methyl-3-phenyl-1H-pyrazol-4-yl)quinazolin-6-yl)methanol (Example 99, 300 mg, 0.832 mmol) and TEA (251 mg, 2.49 mmol) in DCM (10 mL) and the reaction was stirred at rt for 1 h. The reaction was extracted with EtOAc (3×50 mL), the combined organic layer was dried over Na₂SO₄ and concentrated under vacuum. The crude product was purified by Prep-TLC with DCM:MeOH=20:1 to afford the title compound (200 mg) as yellow solid. LCMS: m/z=439 [M+H]⁺.

### Preparation 188: tert-butyl(S)-4-(7-ethoxy-4-(1-methyl-3-phenyl-1H-pyrazol-4-yl)quinazolin-6-yl)-3-methylpiperazine-1-carboxylate

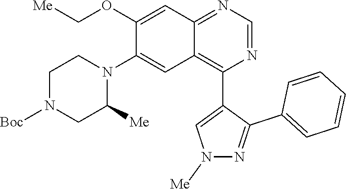 [SMILES: CCOc1cc2ncnc(-c3cn(C)nc3-c3ccccc3)c2cc1N1CCN(C(=O)OC(C)(C)C)C[C@@H]1C]

PEPPSI Pd-Ipent-O-Picoline (40.9 mg, 0.049 mmol) and Cs₂CO₃ (318 mg, 0.976 mmol) were added to 6-bromo-7-ethoxy-4-(1-methyl-3-phenyl-1H-pyrazol-4-yl)quinazoline (Preparation 90, 200 mg, 0.488 mmol) and tert-butyl (3S)-3-methylpiperazine-1-carboxylate (195 mg, 0.976 mmol) in dioxane (10 mL) and the reaction was heated to 80° C. for 16 h under N₂. The reaction mixture was diluted with EtOAc (100 mL) and washed sequentially with water (100 mL×3) and saturated brine (100 mL). The organic layer was dried over Na₂SO₄, filtered and evaporated under reduced pressure. The crude product was purified by Prep-TLC with DCM:MeOH=20:1 to give the title compound, 220 mg, 85.6%, as a light yellow solid. LCMS m/z=529 [M+H]⁺.

### Preparation 189: 4-bromo-7-methoxy-6-nitroquinazoline

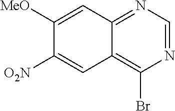 [SMILES: COc1cc2ncnc(Br)c2cc1[N+](=O)[O-]]

Phosphoroyl tribromide (2.59 g, 9.04 mmol) was added to 7-methoxy-6-nitro-3,4-dihydroquinazolin-4-one (1 g, 4.52 mmol) in MeCN (10 mL) at rt and the resulting mixture heated to 90° C. for 1 h. The reaction mixture was poured into 100 g of crushed ice and extracted with EtOAc (3×50 mL). The combined organics were dried (Na₂SO₄) and evaporated to dryness in vacuo. The residue was purified by column chromatography (SiO₂, 33% EtOAc/PE) to afford the title compound as a light yellow solid (350 mg) which was used which was used without further purification.

### Preparation 190: 7-methoxy-4-(1-methyl-3-phenyl-1H-pyrazol-4-yl)quinazolin-6-amine

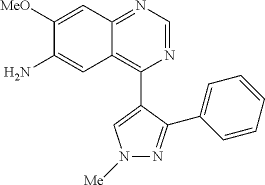 [SMILES: COc1cc2ncnc(-c3cn(C)nc3-c3ccccc3)c2cc1N]

Part 1: A mixture of 4-bromo-7-methoxy-6-nitroquinazoline (Preparation 189, 350 mg, 1.23 mmol), 1-methyl-3-phenyl-4-(4,4,5,5-tetramethyl-1,3,2-dioxaborolan-2-yl)-1H-pyrazole (417 mg, 1.47 mmol), K₂CO₃ (344 mg, 2.46 mmol) and Pd(dppf)Cl₂ (100 mg, 0.123 mmol) in dioxane/water (10 mL/2.5 mL) was heated at 80° C. for 16 h under N₂. The reaction mixture was extracted with EtOAc (3×30 mL) and the combined organics dried (Na₂SO₄) and evaporated to dryness in vacuo. The residue was purified by prep-TLC (15:1 DCM/MeOH) to afford 7-methoxy-4-(1-methyl-3-phenyl-1H-pyrazol-4-yl)-6-nitroquinazoline as a light yellow solid (300 mg). LCMS: m/z=362 [M+H]⁺.

Part 2: A mixture of 7-methoxy-4-(1-methyl-3-phenyl-1H-pyrazol-4-yl)-6-nitroquinazoline (Part 1, 300 mg, 0.830 mmol), iron (463 mg, 8.30 mmol) and NH₄Cl (448 mg, 8.30 mmol) in EtOH/H₂O (10 mL/2.5 mL) was heated to 80° C. for 1 h. The reaction mixture was filtered through a Celite® pad and the filtrate evaporated to dryness in vacuo. The residue was purified by prep-TLC (12:1 DCM/MeOH) to afford the title compound as a light yellow solid (250 mg). LCMS: m/z=332 [M+H]⁺.

### Preparation 191: tert-butyl (2S,5S)-4-((7-methoxy-4-(1-methyl-3-phenyl-1H-pyrazol-4-yl)quinazolin-6-yl)carbamoyl)-2,5-dimethylpiperazine-1-carboxylate

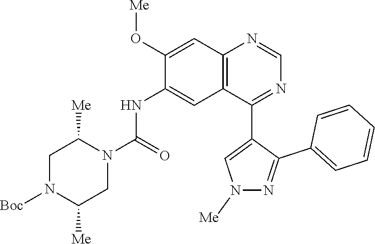 [SMILES: COc1cc2ncnc(-c3cn(C)nc3-c3ccccc3)c2cc1NC(=O)N1C[C@H](C)N(C(=O)OC(C)(C)C)C[C@@H]1C]

To a solution of 7-methoxy-4-(1-methyl-3-phenyl-1H-pyrazol-4-yl)quinazolin-6-amine (Preparation 190, 70.0 mg, 0.211 mmol) in DCM (2 mL) was added pyridine (33.42 mg, 0.422 mmol) and 4-nitrophenyl chloroformate (63.87 mg, 0.317 mmol) at 0° C. and the reaction was stirred at 25° C. for 1 h. The mixture was added dropwise to a solution of tert-butyl (2S,5S)-2,5-dimethylpiperazine-1-carboxylate (226.35 mg, 1.06 mmol) and TEA (64.13 mg, 0.634 mmol) in DCM (2 mL) and the reaction was stirred at 45° C. for 16 h. The reaction mixture was concentrated under reduced pressure and the residue was purified by prep-TLC (PE/EtOAc=0:1) to give the title compound (70 mg, 58%) as yellow gum. ¹H-NMR (400 MHz, CDCl₃) δ ppm 9.08-9.02 (m, 1H), 8.72 (s, 1H), 7.86 (s, 1H), 7.41-7.38 (m, 2H), 7.34 (s, 1H), 7.22-7.14 (m, 4H), 4.28 (d, 1H), 4.08 (d, 6H), 4.00-3.94 (m, 1H), 2.98 (d, 2H), 2.89-2.82 (m, 2H), 1.25 (d, 6H), 1.20 (s, 9H).

### Preparation 192: tert-butyl(S)-4-((7-methoxy-4-(1-methyl-3-phenyl-1H-pyrazol-4-yl)quinazolin-6-yl)carbamoyl)-3-methylpiperazine-1-carboxylate

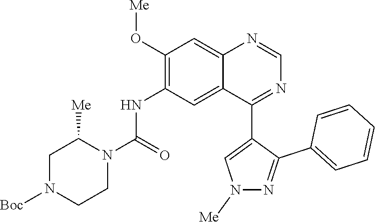 [SMILES: COc1cc2ncnc(-c3cn(C)nc3-c3ccccc3)c2cc1NC(=O)N1CCN(C(=O)OC(C)(C)C)C[C@@H]1C]

The title compound was obtained as a yellow solid, 50 mg, 30% yield, from 7-methoxy-4-(1-methyl-3-phenyl-1H-pyrazol-4-yl)quinazolin-6-amine (Preparation 190), 4-nitrophenyl chloroformate and tert-butyl (3S)-3-methylpiperazine-1-carboxylate following a similar procedure to that described in Preparation 191.

### Preparation 193: tert-butyl (2R,5S)-4-((7-methoxy-4-(1-methyl-3-phenyl-1H-pyrazol-4-yl)quinazolin-6-yl)carbamoyl)-2,5-dimethylpiperazine-1-carboxylate

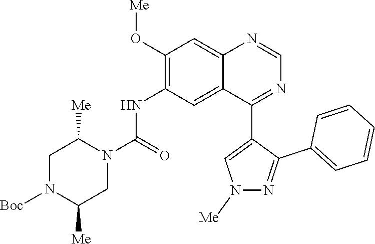 [SMILES: COc1cc2ncnc(-c3cn(C)nc3-c3ccccc3)c2cc1NC(=O)N1C[C@@H](C)N(C(=O)OC(C)(C)C)C[C@@H]1C]

The title compound was obtained as a yellow solid, 190 mg, 55% yield, from 7-methoxy-4-(1-methyl-3-phenyl-1H-pyrazol-4-yl)quinazolin-6-amine (Preparation 190), 4-nitrophenyl chloroformate and tert-butyl (2R,5S)-2,5-dimethylpiperazine-1-carboxylate, following a similar procedure to that described in Preparation 191.

### Preparation 194: tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate or tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate

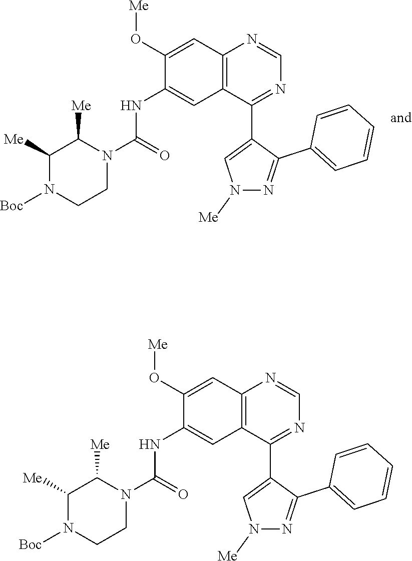 [SMILES: COc1cc2ncnc(-c3cn(C)nc3-c3ccccc3)c2cc1NC(=O)N1CCN(C(=O)OC(C)(C)C)[C@@H](C)[C@H]1C ; COc1cc2ncnc(-c3cn(C)nc3-c3ccccc3)c2cc1NC(=O)N1CCN(C(=O)OC(C)(C)C)[C@H](C)[C@@H]1C]

The racemate of the title compound was obtained as a yellow solid from 7-methoxy-4-(1-methyl-3-phenyl-1H-pyrazol-4-yl)quinazolin-6-amine (Preparation 190), 4-nitrophenyl chloroformate and cis-tert-butyl 2,3-dimethylpiperazine-1-carboxylate following a similar procedure to that described in Preparation 191. LCMS m/z=486 [M+H]⁺. This was further purified by Prep-Chiral-SFC Column: DAICEL CHIRALCEL OJ (250 mm*30 mm, 10 μm); Mobile phase: A for CO₂ and B for IPA (0.1% NH₄OH); Gradient: B %=15% isocratic elution mode; Flow rate: 60 g/min, to give Peak 1, (10.0 mg, 6%) and Peak 2 (10.0 mg, 6%), Preparation 194, tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate or tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate. ¹H-NMR (400 MHZ, CDCl₃) δ ppm 8.99 (s, 1H), 8.63 (s, 1H), 7.80 (s, 1H), 7.31-7.34 (m, 2H), 7.16-7.22 (m, 2H), 7.10-7.13 (m, 3H), 4.21-4.25 (m, 1H), 4.03 (s, 1H), 4.01 (s, 3H), 4.00 (s, 3H), 3.72-3.90 (m, 2H), 3.55-3.63 (m, 1H), 3.36-3.47 (m, 2H), 1.41 (s, 9H), 1.27 (d, 3H), 1.23 (d, 3H).

### Preparation 195: (2S,5S)—N-(7-methoxy-4-(1-methyl-3-phenyl-1H-pyrazol-4-yl)quinazolin-6-yl)-2,5-dimethylpiperazine-1-carboxamide formate

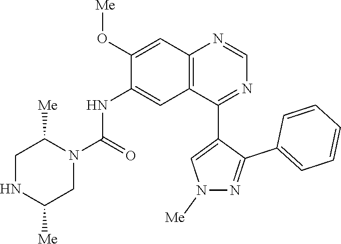 [SMILES: COc1cc2ncnc(-c3cn(C)nc3-c3ccccc3)c2cc1NC(=O)N1C[C@H](C)NC[C@@H]1C]

tert-Butyl (2S,5S)-4-((7-methoxy-4-(1-methyl-3-phenyl-1H-pyrazol-4-yl)quinazolin-6-yl)carbamoyl)-2,5-dimethylpiperazine-1-carboxylate (Preparation 191, 70.0 mg, 0.122 mmol) in HCl/EtOAc (10 mL) was stirred at 25° C. for 5 h. The reaction mixture was concentrated under reduced pressure and purified by prep-HPLC (Column: Phenomenex Luna C18 200*40 mm*10 um; mobile phase: [water (0.2% FA)-MeCN]; B %: 1%-50%, 8 min) to give the title compound as pale yellow solid (11.7 mg, 18%). LCMS m/z=472 [M+H]⁺. ¹H-NMR (400 MHZ, CDCl₃) δ ppm 9.07 (s, 1H), 8.67 (s, 1H), 7.87 (s, 1H), 7.38-7.44 (m, 2H), 7.36 (s, 1H), 7.25 (s, 2H), 7.19 (d, 3H), 4.15-4.26 (m, 1H), 4.10 (s, 3H), 4.08 (s, 3H), 3.83 (d, 1H), 2.98-3.15 (m, 2H), 2.75-2.96 (m, 2H), 1.38 (d, 3H), 1.27 (d, 3H).

### Preparation 196: (2R,3S)—N-(7-methoxy-4-(1-methyl-3-phenyl-1H-pyrazol-4-yl)quinazolin-6-yl)-2,3-dimethylpiperazine-1-carboxamide or (2S,3R)—N-(7-methoxy-4-(1-methyl-3-phenyl-1H-pyrazol-4-yl)quinazolin-6-yl)-2,3-dimethylpiperazine-1-carboxamide

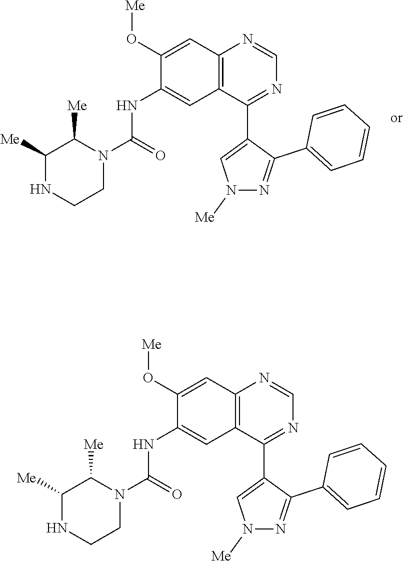 [SMILES: COc1cc2ncnc(-c3cn(C)nc3-c3ccccc3)c2cc1NC(=O)N1CCN[C@@H](C)[C@H]1C ; COc1cc2ncnc(-c3cn(C)nc3-c3ccccc3)c2cc1NC(=O)N1CCN[C@H](C)[C@@H]1C]

A solution of tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate or tert-butyl (2S,3R)-4-((7-methoxy-4-(1-methyl-3-phenyl-1H-pyrazol-4-yl)quinazolin-6-yl)carbamoyl)-2,3-dimethylpiperazine-1-carboxylate Preparation 194 (10.0 mg, 0.0175 mmol) in HCl/EtOAc (2 mL) was stirred at 25° C. for 1 h. The reaction mixture was concentrated under reduced pressure. The residue was purified by prep-HPLC (Column: Phenomenex Gemini-NX C18 75*30 mm*3 um; mobile phase: [water-(0.05% NH₃H₂O₄ 10 mM NH₄HCO₃)—MeCN]; B %: 20%-45%, 8 min) to give the title compound as a white solid (2.60 mg, 30%). LCMS m/z=472 [M+H]⁺. ¹H-NMR (400 MHZ, CDCl₃) δ ppm 9.04 (s, 1H), 8.67 (s, 1H), 7.84 (s, 1H), 7.37-7.41 (m, 2H), 7.31 (s, 1H), 7.22-7.24 (m, 1H), 7.15-7.18 (m, 3H), 4.06 (s, 3H), 3.99-4.05 (m, 5H), 3.63-3.74 (m, 1H), 2.98-3.17 (m, 3H), 2.81-2.88 (m, 1H), 1.18 (d, 3H, J=6.8 Hz), 1.10 (d, 3H, J=7.2 Hz).

### Preparation 197: (S)—N-(7-methoxy-4-(1-methyl-3-phenyl-1H-pyrazol-4-yl)quinazolin-6-yl)-2-methylpiperazine-1-carboxamide trifluoroacetate

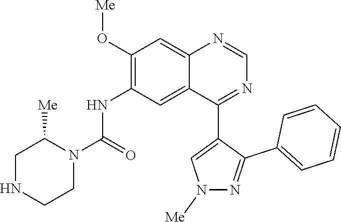 [SMILES: COc1cc2ncnc(-c3cn(C)nc3-c3ccccc3)c2cc1NC(=O)N1CCNC[C@@H]1C]

A mixture of tert-butyl(S)-4-((7-methoxy-4-(1-methyl-3-phenyl-1H-pyrazol-4-yl)quinazolin-6-yl)carbamoyl)-3-methylpiperazine-1-carboxylate (Preparation 192, 50 mg, 0.090 mmol) in TFA (0.2 mL) and DCM (1 mL) was stirred at 25° C. for 1 h. The reaction mixture was concentrated under reduced pressure to afford the title compound as yellow oil (20 mg, crude).

### Preparation 198: (2S,5R)—N-(7-methoxy-4-(1-methyl-3-phenyl-1H-pyrazol-4-yl)quinazolin-6-yl)-2,5-dimethylpiperazine-1-carboxamide

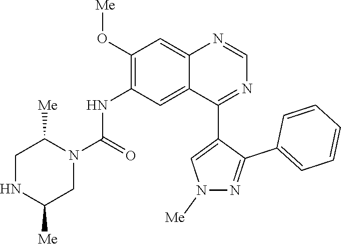 [SMILES: COc1cc2ncnc(-c3cn(C)nc3-c3ccccc3)c2cc1NC(=O)N1C[C@@H](C)NC[C@@H]1C]

A mixture of the tert-butyl (2R,5S)-4-((7-methoxy-4-(1-methyl-3-phenyl-1H-pyrazol-4-yl)quinazolin-6-yl)carbamoyl)-2,5-dimethylpiperazine-1-carboxylate (Preparation 193, 190 mg, 0.332 mmol) in DCM (2 mL) and TFA (0.4 mL) was stirred at 25° C. for 1 h. The reaction mixture concentrated under reduced pressure. The residue was purified by prep-HPLC (Column: Waters Xbridge BEH C18 100*30 mm*10 um; mobile phase: [water (10 mM NH₄HCO₃)—MeCN]; B %: 12%-42%, 10 min) to give the title compound as a yellow solid (22.1 mg, 13% yield). LCMS m/z=472 [M+H]⁺. ¹H-NMR (400 MHZ, CDCl₃) δ ppm 9.10 (s, 1H), 8.69 (s, 1H), 8.17 (d, 1H), 7.90 (s, 1H), 7.41 (dd, 2H), 7.38 (s, 1H), 7.22-7.20 (m, 3H), 6.89 (d, 1H), 4.23 (s, 1H), 4.11 (d, 6H), 3.62 (d, 1H), 3.52-3.47 (m, 1H), 3.44 (s, 1H), 3.37 (dd, 1H), 2.79 (dd, 1H), 1.40 (d, 3H), 1.34 (d, 3H).

### Preparation 199: N-(7-methoxy-4-oxo-3,4-dihydroquinazolin-6-yl)cyclopropanecarboxamide

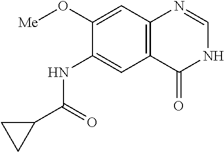 [SMILES: COc1cc2nc[nH]c(=O)c2cc1NC(=O)C1CC1]

Cyclopropanecarbonyl chloride (6.54 g, 62.6 mmol) was added dropwise to 6-amino-7-methoxy-3,4-dihydroquinazolin-4-one (4 g, 20.9 mmol) and TEA (11.9 g, 104 mmol) in DCM (35 mL) at 0° C. The resulting mixture was stirred at rt for 1 h. The resulting solid was filtered and washed with EtOAc (20 mL) to give the title compound (3.9 g, yield: 97%) as a yellow solid. LCMS m/z=260 [M+H]⁺.

### Preparation 200: N-(7-methoxy-4-oxo-3,4-dihydroquinazolin-6-yl)propionamide

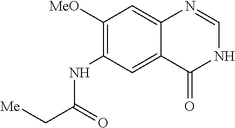 [SMILES: CCC(=O)Nc1cc2c(=O)[nH]cnc2cc1OC]

Part 1. Fe (804 mg, 14.4 mmol) and NH₄Cl (163 mg, 14.4 mmol) were added to 7-methoxy-6-nitroquinazolin-4 (3H)-one (400 mg, 1.8 mmol) in EtOH/H₂O (10 mL/2 mL) and the resulting mixture was heated to 80° C. for 2 h. The reaction was filtered and the filtrate was concentrated in vacuo and the residue purified by prep-TLC (15:1 DCM/MeOH) to afford 6-amino-7-methoxyquinazolin-4 (3H)-one as a white solid (300 mg) which was used without further purification.

Part 2. To a solution of 6-amino-7-methoxyquinazolin-4 (3H)-one (Part 1, 300 mg, 1.56 mmol) in propionic anhydride (5 mL) was heated to 120° C. for 2 h. The reaction was evaporated to dryness in vacuo and the residue purified by prep-TLC (15:1 DCM/MeOH) to afford the title compound as a white solid (180 mg, 40%). LCMS: m/z=248 [M+H]⁺.

### Preparation 201: N-(4-chloro-7-methoxyquinazolin-6-yl)cyclopropanecarboxamide

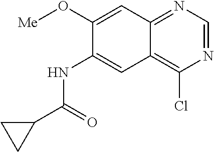 [SMILES: COc1cc2ncnc(Cl)c2cc1NC(=O)C1CC1]

POCl₃ (1.46 g, 9.60 mmol) and TEA (1.17 g, 11.5 mmol) were added to N-(7-methoxy-4-oxo-3,4-dihydroquinazolin-6-yl)cyclopropanecarboxamide (Preparation 199, 500 mg, 1.92 mmol) in MeCN (15 mL) at rt. The reaction mixture was heated to 80° C. for 2 h, then poured onto ice-water. Na₂CO₃ (1 M) was added to achieve pH 8. The resulting solution was extracted with EtOAc and concentrated in vacuo. The crude product was purified by column chromatography (SiO₂, 5:1 DCM/EtOAc) to afford the title compound as a yellow solid (400 mg, 75%). LCMS m/z=278 [M+H]⁺.

### Preparation 202: N-(4-chloro-7-methoxyquinazolin-6-yl)propionamide

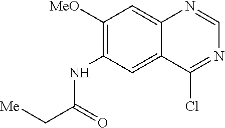 [SMILES: CCC(=O)Nc1cc2c(Cl)ncnc2cc1OC]

POCl₃ (167 mg, 1.09 mmol) was added to N-(7-methoxy-4-oxo-3,4-dihydroquinazolin-6-yl)propionamide (Preparation 200, 180 mg, 0.727 mmol) in MeCN (5 mL) was heated to 100° C. for 1 h. The reaction was quenched with H₂O (2 mL) at 0° C. and extracted with EtOAc (3×15 mL). The combined organics were dried (Na₂SO₄) and evaporated to dryness in vacuo. The residue was purified by prep-TLC (15:1 DCM/MeOH) to afford the title compound as a pale yellow solid (60 mg, 31%). LCMS: m/z=266 [M+H]⁺.

### Preparation 203: 4-chloro-7-methoxy-6-nitroquinazoline

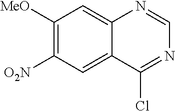 [SMILES: COc1cc2ncnc(Cl)c2cc1[N+](=O)[O-]]

7-Methoxy-6-nitroquinazolin-4-ol (10 g, 45.2 mmol) was suspended in POCl₃ (165 g, 1.08 mol) and the mixture heated to 110° C. for 4 h. The reaction mixture was evaporated to dryness in vacuo and the residue dissolved in a mixture of DCM (50 mL) and aqueous NaHCO₃ (50 mL). The organic layer was dried and evaporated to dryness to afford the title compound as a yellow solid (7 g, 58%) which was used without further purification.

### Preparation 204: 7-methoxy-4-(1-methyl-3-phenyl-1H-pyrazol-4-yl)-6-nitroquinazoline

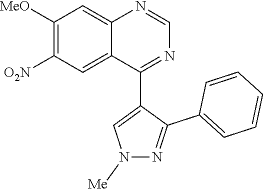 [SMILES: COc1cc2ncnc(-c3cn(C)nc3-c3ccccc3)c2cc1[N+](=O)[O-]]

To a mixture of 4-chloro-7-methoxy-6-nitroquinazoline (Preparation 203, 1.07 g, 3.76 mmol), 1-methyl-3-phenyl-4-(4,4,5,5-tetramethyl-1,3,2-dioxaborolan-2-yl)-1H-pyrazole (1 g, 3.76 mmol) and Cs₂CO₃ (2.45 g, 7.51 mmol) in dioxane (10 mL), H₂O (2 mL) was added Pd-118 (122.40 mg, 0.188 mmol) and the mixture stirred at 100° C. for 2 h. The reaction mixture was concentrated under reduced pressure and the residue purified by silica gel chromatography (ISCOR: 0-30% EtOAc/PE) to afford the title compound as a yellow solid (1 g, 66%).

### Preparation 205: 4-(1-ethyl-3-(4-fluorophenyl)-1H-pyrazol-4-yl)-7-methoxy-6-nitroquinazoline

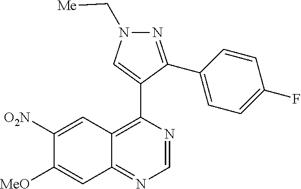 [SMILES: CCn1cc(-c2ncnc3cc(OC)c([N+](=O)[O-])cc23)c(-c2ccc(F)cc2)n1]

To a mixture of 4-bromo-7-methoxy-6-nitroquinazoline (Preparation 189, 200 mg, 0.704 mmol) and 1-ethyl-3-(4-fluorophenyl)-4-(4,4,5,5-tetramethyl-1,3,2-dioxaborolan-2-yl)-1H-pyrazole (Preparation 44, 222 mg, 0.704 mmol) in dioxane (8 mL) and H₂O (2 mL) was added Pd(dppf)Cl₂ (25.7 mg, 0.0352 mmol) and K₂CO₃ (193 mg, 1.40 mmol) at 25° C. and the mixture was stirred at 80° C. for 4 h under N₂. The reaction mixture was diluted with water (40 mL) and extracted with EtOAc (2×40 mL). The combined organics were washed with brine (20 mL), dried (Na₂SO₄) and evaporated to dryness in vacuo. The residue was purified by prep-TLC (20:1 DCM/MeOH) to afford the title compound as a yellow solid (120 mg, 43%). LCMS: m/z=394 [M+H]⁺.

### Preparation 206: N-(7-methoxy-4-(3-phenyl-1-(tetrahydro-2H-pyran-2-yl)-1H-pyrazol-4-yl)quinazolin-6-yl)propionamide

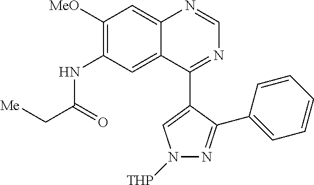

To a solution of N-(4-chloro-7-methoxyquinazolin-6-yl)propionamide (Preparation 202, 60 mg, 0.225 mmol), 3-phenyl-1-(tetrahydro-2H-pyran-2-yl)-4-(4,4,5,5-tetramethyl-1,3,2-dioxaborolan-2-yl)-1H-pyrazole (Preparation 42, 87.5 mg, 0.247 mmol), K₂CO₃ (63 mg, 0.450 mmol) and Pd(dppf)Cl₂ (18.3 mg, 0.0225 mmol) in dioxane/water (3 mL/0.8 mL) was heated at 80° C. for 2 h under N₂. The reaction was evaporated to dryness in vacuo and the residue purified by prep-TLC (15:1 DCM/MeOH) to afford the title compound as a yellow solid (20 mg, 19%). LCMS: m/z=458 [M+H]⁺.

### Preparation 207: N-(4-(1-(2-((tert-butyldimethylsilyl)oxy)ethyl)-3-phenyl-1H-pyrazol-4-yl)-7-methoxyquinazolin-6-yl)-2-fluorobenzamide

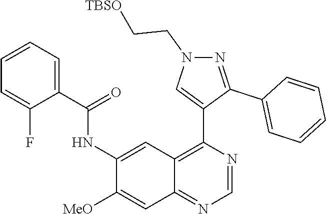 [SMILES: COc1cc2ncnc(-c3cn(CCO[Si](C)(C)C(C)(C)C)nc3-c3ccccc3)c2cc1NC(=O)c1ccccc1F]

A mixture of 6-bromo-4-(1-(2-((tert-butyldimethylsilyl)oxy)ethyl)-3-phenyl-1H-pyrazol-4-yl)-7-methoxyquinazoline (Preparation 106, 100 mg, 0.167 mmol), 2-fluorobenzamide (34.7 mg, 0.250 mmol), Pd₂ (dba); (20 mg, 0.0193 mmol), XPhos (20 mg, 0.0345 mmol) and Cs₂CO₃ (108 mg, 0.334 mmol) in toluene (10 mL) was stirred at 100° C. for 2 h. The reaction mixture was evaporated to dryness in vacuo and the residue purified by prep-TLC (35:1 DCM/MeOH) to afford the title compound as a yellow solid (84 mg, 73%). LCMS: m/z=598 [M+H]⁺.

### Preparation 208: N-(4-(1-(2-((tert-butyldimethylsilyl)oxy)ethyl)-3-phenyl-1H-pyrazol-4-yl)-7-methoxyquinazolin-6-yl)bicyclo[1.1.1]pentane-1-carboxamide

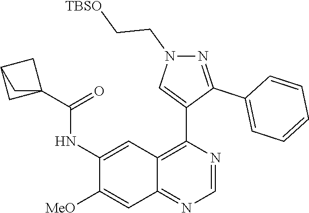 [SMILES: COc1cc2ncnc(-c3cn(CCO[Si](C)(C)C(C)(C)C)nc3-c3ccccc3)c2cc1NC(=O)C12CC(C1)C2]

The title compound was prepared as a yellow solid (159 mg, 86%) from 6-bromo-4-(1-(2-((tert-butyldimethylsilyl)oxy)ethyl)-3-phenyl-1H-pyrazol-4-yl)-7-methoxyquinazoline (Preparation 106) and bicyclo[1.1.1]pentane-1-carboxamide (Preparation 70) using an analogous method to that described for Preparation 207. LCMS: m/z=570 [M+H]⁺.

### Preparation 209: 6-chloro-7-methoxypyrido[3,2-d]pyrimidin-4 (3H)-one

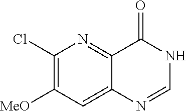 [SMILES: COc1cc2nc[nH]c(=O)c2nc1Cl]

Formamidine acetate (420 g, 4.04 mol) was added to 3-amino-6-chloro-5-methoxypicolinic acid (220 g, 1.01 mol) in EtOH (2.0 L) at rt and the resulting mixture was heated to 90° C. for 24 h. The solids were collected by filtration to afford the title compound as a brown solid (201 g, 94%). LCMS: m/z=212 [M+H]⁺.

### Preparation 210: 7-methoxy-6-(4-methoxybenzyl)amino)pyrido[3,2-d]pyrimidin-4 (3H)-one

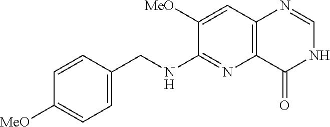 [SMILES: COc1ccc(CNc2nc3c(=O)[nH]cnc3cc2OC)cc1]

1-(4-Methoxyphenyl) methanamine (45.2 g, 330 mmol) was added to 6-chloro-7-methoxypyrido[3,2-d]pyrimidin-4 (3H)-one (Preparation 209, 10 g, 47.2 mmol) in DMSO (50 mL) at rt and the resulting mixture stirred at 100° C. for 2 h. The mixture was diluted with EtOAc (100 mL) and washed with brine (2×50 mL). The combined organics were dried (Na₂SO₄) and evaporated to dryness in vacuo. The residue was purified by column chromatography (SiO₂, 25:1 DCM/MeOH) to afford the title compound as a yellow solid (8 g, 54%). LCMS: m/z=313 [M+H]⁺.

### Preparation 211: 4-chloro-7-methoxy-N-(4-methoxybenzyl)pyrido[3,2-d]pyrimidin-6-amine

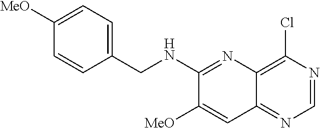 [SMILES: COc1ccc(CNc2nc3c(Cl)ncnc3cc2OC)cc1]

POCl₃ (15.7 g, 102 mmol) and N,N-diethylaniline (25.7 g, 153 mmol) were added to 7-methoxy-6-((4-methoxybenzyl)amino)pyrido[3,2-d]pyrimidin-4 (3H)-one (Preparation 210, 8 g, 25.6 mmol) in MeCN (15 mL) at rt and the reaction stirred at 80° C. for 2 h. The resulting mixture was added to ice-water and the pH adjusted to pH 8 using aq. Na₂CO₃ (1 M) and extracted with EtOAc (2×500 mL). The combined organic extracts were dried (Na₂SO₄) and evaporated to dryness in vacuo. The residue was purified by silica gel chromatography (10:1 DCM/EtOAc) to afford the title compound as a yellow solid (7 g, 83%). LCMS: m/z=331 [M+H]⁺.

### Preparation 212:7-methoxy-N-(4-methoxybenzyl)-4-(1-methyl-3-phenyl-1H-pyrazol-4-yl)pyrido[3,2-d]pyrimidin-6-amine

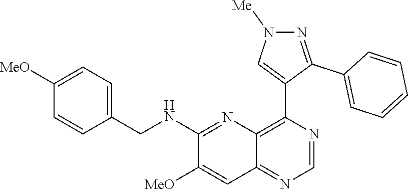 [SMILES: COc1ccc(CNc2nc3c(-c4cn(C)nc4-c4ccccc4)ncnc3cc2OC)cc1]

A mixture of Pd(dppf)Cl₂ (1.76 g, 2.11 mmol), K₂CO₃ (4.36 g, 31.6 mmol), 4-chloro-7-methoxy-N-(4-methoxybenzyl)pyrido[3,2-d]pyrimidin-6-amine (Preparation 211, 7 g, 21.1 mmol) and 1-methyl-3-phenyl-4-(4,4,5,5-tetramethyl-1,3,2-dioxaborolan-2-yl)-1H-pyrazole (8.97 g, 31.6 mmol) in H₂O (30 mL) and dioxane (120 mL) was stirred at 80° C. for 2 h under N₂. The mixture was diluted with EtOAc (200 mL) and washed with water (2×50 mL). The combined organics were dried (Na₂SO₄) and evaporated to dryness in vacuo. The residue was purified by silica gel chromatography (20:1 DCM/MeOH) to afford the title compound as a yellow oil (6.5 g, 68%). LCMS: m/z=453 [M+H]⁺.

### Preparation 213: 4-(3-(2,4-difluorophenyl)-1-methyl-1H-pyrazol-4-yl)-7-methoxy-N-(4-methoxybenzyl)pyrido[3,2-d]pyrimidin-6-amine

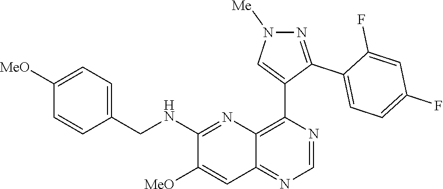 [SMILES: COc1ccc(CNc2nc3c(-c4cn(C)nc4-c4ccc(F)cc4F)ncnc3cc2OC)cc1]

The title compound was prepared as an off-white solid (550 mg, 80%) from 4-chloro-7-methoxy-N-(4-methoxybenzyl)pyrido[3,2-d]pyrimidin-6-amine (Preparation 211) and 3-(2,4-difluorophenyl)-1-methyl-4-(4,4,5,5-tetramethyl-1,3,2-dioxaborolan-2-yl)-1H-pyrazole (Preparation 45) using an analogous method to that described for Preparation 212. LCMS: m/z=489 [M+H]⁺.

### Preparation 214: 4-(3-(4-fluorophenyl)-1-methyl-1H-pyrazol-4-yl)-7-methoxy-N-(4-methoxybenzyl)pyrido[3,2-d]pyrimidin-6-amine

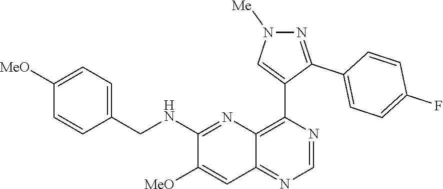 [SMILES: COc1ccc(CNc2nc3c(-c4cn(C)nc4-c4ccc(F)cc4)ncnc3cc2OC)cc1]

The title compound was prepared as a yellow solid (600 mg, 85%) from 4-chloro-7-methoxy-N-(4-methoxybenzyl)pyrido[3,2-d]pyrimidin-6-amine (Preparation 211) and 3-(4-fluorophenyl)-1-methyl-4-(4,4,5,5-tetramethyl-1,3,2-dioxaborolan-2-yl)-1H-pyrazole (Preparation 43) using an analogous method to that described for Preparation 212. LCMS: m/z=471 [M+H]⁺.

### Preparation 215:4-(3-(3-((tert-butyldimethylsilyl)oxy)phenyl)-1-methyl-1H-pyrazol-4-yl)-7-methoxy-N-(4-methoxybenzyl)pyrido[3,2-d]pyrimidin-6-amine

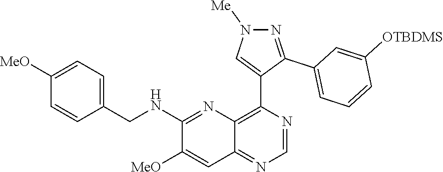 [SMILES: COc1ccc(CNc2nc3c(-c4cn(C)nc4-c4cccc(O[Si](C)(C)C(C)(C)C)c4)ncnc3cc2OC)cc1]

A mixture of Pd(PPh₃)₄ (138 mg, 0.120 mmol), K₃PO₄ (381 mg, 1.79 mmol), 4-chloro-7-methoxy-N-(4-methoxybenzyl)pyrido[3,2-d]pyrimidin-6-amine (Preparation 211, 396 mg, 1.20 mmol) and 3-(3-((tert-butyldimethylsilyl)oxy)phenyl)-1-methyl-4-(4,4,5,5-tetramethyl-1,3,2-dioxaborolan-2-yl)-1H-pyrazole (Preparation 39, 500 mg, 1.20 mmol) in dioxane/H₂O (8 mL/2 mL) was stirred at 80° C. for 2 h under N₂. The resulting solution was extracted with EtOAc (3×50 mL) and the combined organic extracts dried (Na₂SO₄) and evaporated to dryness in vacuo. The residue was purified by silica gel chromatography (25:1 DCM/MeOH) to afford the title compound as a yellow solid (370 mg, 52%). LCMS: m/z=583 [M+H]⁺.

### Preparation 216: 7-methoxy-4-(1-methyl-3-phenyl-1H-pyrazol-4-yl)pyrido[3,2-d]pyrimidin-6-amine

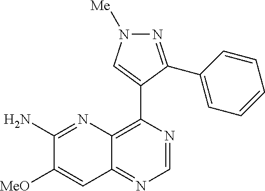 [SMILES: COc1cc2ncnc(-c3cn(C)nc3-c3ccccc3)c2nc1N]

TFA (5 mL) was added to 7-methoxy-N-(4-methoxybenzyl)-4-(1-methyl-3-phenyl-1H-pyrazol-4-yl)pyrido[3,2-d]pyrimidin-6-amine (Preparation 212, 3 g, 5.49 mmol) at rt and the mixture stirred at 60° C. for 3 h. The mixture was diluted with EtOAc (100 mL), washed with brine (2×50 mL), dried (Na₂SO₄) and evaporated to dryness in vacuo. The residue was purified by silica gel chromatography (18:1 DCM/MeOH) to afford the title compound as a yellow solid (2.5 g). LCMS: m/z=333 [M+H]⁺.

### Preparation 217: 4-(3-(2,4-difluorophenyl)-1-methyl-1H-pyrazol-4-yl)-7-methoxypyrido[3,2-d]pyrimidin-6-amine

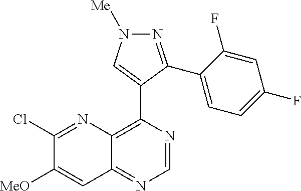 [SMILES: COc1cc2ncnc(-c3cn(C)nc3-c3ccc(F)cc3F)c2nc1Cl]

A mixture of 4-(3-(2,4-difluorophenyl)-1-methyl-1H-pyrazol-4-yl)-7-methoxy-N-(4-methoxybenzyl)pyrido[3,2-d]pyrimidin-6-amine (Preparation 213, 550 mg, 1.12 mmol) and TFA (5 mL) was stirred at 60° C. for 3 h under N₂. The mixture was diluted with EtOAc (100 mL) and washed with NaHCO_{3(aq)} (2×50 mL). The combined organics were dried (Na₂SO₄), evaporated to dryness in vacuo and the residue was purified by silica gel chromatography (18:1 DCM/EtOAc) to afford the title compound as a yellow solid (320 mg, 78%). LCMS: m/z=369 [M+H]⁺.

### Preparation 218:4-(3-(4-fluorophenyl)-1-methyl-1H-pyrazol-4-yl)-7-methoxypyrido[3,2-d]pyrimidin-6-amine

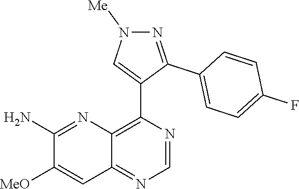 [SMILES: COc1cc2ncnc(-c3cn(C)nc3-c3ccc(F)cc3)c2nc1N]

The title compound was prepared as a yellow solid (400 mg, 90%) from 4-(3-(4-fluorophenyl)-1-methyl-1H-pyrazol-4-yl)-7-methoxy-N-(4-methoxybenzyl)pyrido[3,2-d]pyrimidin-6-amine (Preparation 214) using an analogous method to that described for Preparation 217. LCMS: m/z=351 [M+H]⁺.

### Preparation 219: 3-(4-(6-amino-7-methoxypyrido[3,2-d]pyrimidin-4-yl)-1-methyl-1H-pyrazol-3-yl)phenol

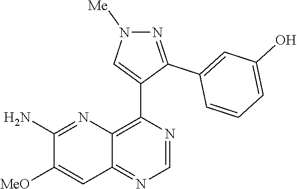 [SMILES: COc1cc2ncnc(-c3cn(C)nc3-c3cccc(O)c3)c2nc1N]

A solution of 4-(3-(3-((tert-butyldimethylsilyl)oxy)phenyl)-1-methyl-1H-pyrazol-4-yl)-7-methoxy-N-(4-methoxybenzyl)pyrido[3,2-d]pyrimidin-6-amine (Preparation 215, 370 mg, 0.634 mmol) in TFA (5 mL) was stirred at 60° C. for 2 h. The resulting solution was extracted with EtOAc (3×50 mL) and the combined extracts dried (Na₂SO₄) and evaporated to dryness. The residue was purified by prep-TLC (15:1 DCM/MeOH) to afford the title compound as a yellow solid (140 mg, 63%). LCMS: m/z=349 [M+H]⁺.

### Preparation 220: 4-(3-(2-fluorophenyl)-1-methyl-1H-pyrazol-4-yl)-7-methoxy-N-(4-methoxybenzyl)pyrido[3,2-d]pyrimidin-6-amine

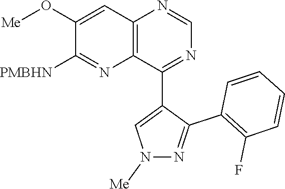

A mixture of 4-chloro-7-methoxy-N-(4-methoxybenzyl)pyrido[3,2-d]pyrimidin-6-amine (Preparation 211, 800 mg, 2.4 mmol), K₂CO₃ (672 mg, 4.8 mmol), Pd(dppf)Cl₂ (174 mg, 0.24 mmol) and 3-(2-fluorophenyl)-1-methyl-4-(4,4,5,5-tetramethyl-1,3,2-dioxaborolan-2-yl)-1H-pyrazole (Preparation 37, 724 mg, 2.4 mmol) in dioxane/H₂O (10 mL/2 mL) was stirred at 80° C. for 1 h. The reaction mixture was concentrated to dryness. The residue was purified by column chromatography (SiO₂, 5% MeOH/DCM) to afford the title compound as an off-white solid (800 mg, 71%). LCMS m/z=471 [M+H]⁺.

### Preparation 221: 4-(3-(2-fluorophenyl)-1-methyl-1H-pyrazol-4-yl)-7-methoxypyrido[3,2-d]pyrimidin-6-amine trifluoroacetate

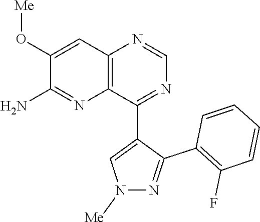 [SMILES: COc1cc2ncnc(-c3cn(C)nc3-c3ccccc3F)c2nc1N]

To a solution of 4-(3-(2-fluorophenyl)-1-methyl-1H-pyrazol-4-yl)-7-methoxy-N-(4-methoxybenzyl)pyrido[3,2-d]pyrimidin-6-amine (Preparation 220, 800 mg, 1.70 mmol) in DCM (5 mL) was added TFA (2 mL) at 0° C. and the reaction mixture was stirred at rt for 3 h. The solution was concentrated to dryness to yield the title compound as a yellow oil (600 mg, 90%). LCMS m/z=351 [M+H]⁺.

### Preparation 222: 4-(1-(2,2-difluoroethyl)-3-phenyl-1H-pyrazol-4-yl)-7-methoxy-N-(4-methoxybenzyl)pyrido[3,2-d]pyrimidin-6-amine

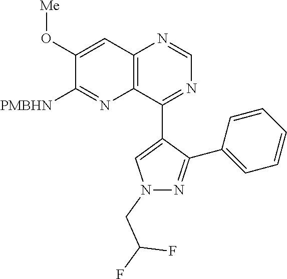

The title compound was obtained as a light yellow solid, 560 mg, 43%, from 4-chloro-7-methoxy-N-(4-methoxybenzyl)pyrido[3,2-d]pyrimidin-6-amine (Preparation 211) and 1-(2,2-difluoroethyl)-3-phenyl-4-(4,4,5,5-tetramethyl-1,3,2-dioxaborolan-2-yl)-1H-pyrazole (Preparation 41) following the procedure described in Preparation 220. LCMS m/z=503 [M+H]⁺.

### Preparation 223: 4-(1-(2,2-difluoroethyl)-3-phenyl-1H-pyrazol-4-yl)-7-methoxypyrido[3,2-d]pyrimidin-6-amine trifluoroaceate

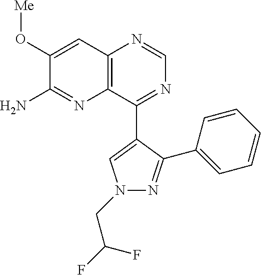 [SMILES: COc1cc2ncnc(-c3cn(CC(F)F)nc3-c3ccccc3)c2nc1N]

The title compound was obtained as a light yellow oil, 400 mg, crude, from 4-(1-(2,2-difluoroethyl)-3-phenyl-1H-pyrazol-4-yl)-7-methoxy-N-(4-methoxybenzyl)pyrido[3,2-d]pyrimidin-6-amine (Preparation 222), following the procedure described in Preparation 221. LCMS m/z=383 [M+H]⁺.

### Preparation 224: 4,6-dichloro-7-methoxypyrido[3,2-d]pyrimidine

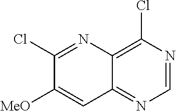 [SMILES: COc1cc2ncnc(Cl)c2nc1Cl]

N,N-Diethylaniline (986 g, 6.61 mol) was added to 6-chloro-7-methoxypyrido[3,2-d]pyrimidin-4 (3H)-one (Preparation 209, 200 g, 945 mmol) and POCl₃ (722 g, 4.72 mol) in MeCN (2000 mL) at rt and the resulting mixture was heated at 80° C. for 2 h. The reaction mixture was added to ice-water and Na₂CO₃ (5 M) added until pH=8. The solution was extracted with EtOAc and the combined organics concentrated under vacuum. The residue was purified by silica gel chromatography (5:1 DCM/EtOAc) to give the title compound as a yellow solid (178 g, 82%). LCMS: m/z=230 [M+H]⁺.

### Preparation 225: 2-bromo-5-ethoxypyridin-3-amine

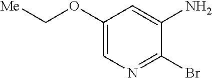 [SMILES: CCOc1cnc(Br)c(N)c1]

NaOEt (45 mL) was added at rt to a solution of 2-bromo-5-fluoropyridin-3-amine (6 g, 31.4 mmol) in EtOH (45 mL) and the mixture was heated at 120° C. for 16 h. After cooling down to 25° C. the mixture was evaporated to dryness and the residue purified by column chromatography (SiO₂, 5:1 PE/EtOAc) to afford the title compound as an off-white solid (5.1 g, 75%). LCMS: m/z=217 [M+H]⁺.

### Preparation 226: methyl 3-amino-5-ethoxypicolinate

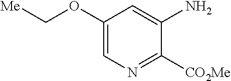 [SMILES: CCOc1cnc(C(=O)OC)c(N)c1]

A mixture of 2-bromo-5-ethoxypyridin-3-amine (Preparation 225, 5.5 g, 25.3 mmol), TEA (7.65 g, 75.8 mmol) and Pd(dppf)Cl₂ (1.84 g, 2.53 mmol) in MeOH (100 mL) was stirred under an atmosphere of CO (10 atm) at 80° C. for 16 h. The reaction mixture was diluted with EtOAc (200 mL), washed with water (3×200 mL) and saturated brine (200 mL). The organic layer was dried (Na₂SO₄) and evaporated to dryness in vacuo. The residue was purified by silica gel column chromatography (20% EtOAc/PE) to afford the title compound as a yellow solid (1.5 g, 30%). LCMS: m/z=197 [M+H]⁺.

### Preparation 227: Methyl 3-amino-6-chloro-5-ethoxypicolinate

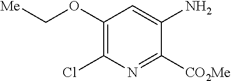 [SMILES: CCOc1cc(N)c(C(=O)OC)nc1Cl]

NCS (1.48 g, 11.1 mmol) was added to a solution of methyl 3-amino-5-ethoxypicolinate (Preparation 226, 2 g, 10.1 mmol) in MeCN (50 mL) at rt and the resulting mixture heated to 80° C. for 16 h. The reaction mixture was diluted with EtOAc (150 mL), washed with water (3×150 mL) and saturated brine (150 mL). The organic layer was dried (Na₂SO₄) and evaporated to dryness in vacuo. The residue was purified by silica gel chromatography (2:1 PE/EtOAc) to afford the title compound as a light yellow solid (1.6 g, 69%). LCMS: m/z=231 [M+H]⁺.

### Preparation 228: 3-amino-6-chloro-5-ethoxypicolinic Acid

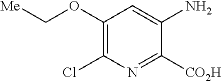 [SMILES: CCOc1cc(N)c(C(=O)O)nc1Cl]

LiOH (996 mg, 41.5 mmol) was added to methyl 3-amino-6-chloro-5-ethoxypicolinate (Preparation 227, 1.6 g, 6.93 mmol) in THF/H₂O (15 mL/5 mL) at rt and the resulting mixture stirred at rt for 16 h. The pH was adjusted to pH 3 with HCl (1 mmol). The reaction mixture was diluted with DCM (200 mL) and washed with water (3×200 mL) and saturated brine (200 mL). The combined organics were dried (Na₂SO₄) and evaporated to dryness in vacuo to afford the title compound as an off-white solid (1.4 g, 93%). LCMS: m/z=217 [M+H]⁺.

### Preparation 229:6-chloro-7-ethoxypyrido[3,2-d]pyrimidin-4 (3H)-one

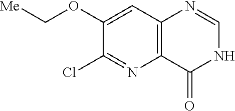 [SMILES: CCOc1cc2nc[nH]c(=O)c2nc1Cl]

Formamidine acetic acid salt (5.75 g, 55.3 mmol) was added to 3-amino-6-chloro-5-ethoxypicolinic acid (Preparation 228, 1.5 g, 6.92 mmol) in EtOH (50 mL) at rt and the resulting mixture heated to 80° C. for 16 h. The reaction mixture was diluted with water (200 mL) and the solids collected by filtration to afford the title compound as a white solid (1.5 g, 96%). LCMS: m/z=226 [M+H]⁺.

### Preparation 230: 4,6-dichloro-7-ethoxypyrido[3,2-d]pyrimidine

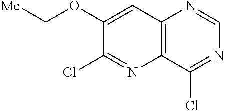 [SMILES: CCOc1cc2ncnc(Cl)c2nc1Cl]

POCl₃ (5.11 g, 33.2 mmol) was added to 6-chloro-7-ethoxypyrido[3,2-d]pyrimidin-4 (3H)-one (Preparation 229, 1.5 g, 6.64 mmol) and N,N-diethylaniline (6.92 g, 46.4 mmol) in MeCN (50 mL) at rt and the resulting mixture heated to 80° C. for 2 h. The solution was poured into ice-water and the pH adjusted to pH 8 by the addition of aq. NaHCO₃ (1.00 M). The solution was extracted with EtOAc and evaporated to dryness in vacuo. The residue was purified by silica gel column chromatography (3:1 DCM:EtOAc) to afford the title compound as an off-white solid (1.4 g, 86%). LCMS: m/z=244 [M+H]⁺.

### Preparation 231: 6-chloro-7-ethoxy-4-(1-methyl-3-phenyl-1H-pyrazol-4-yl)pyrido[3,2-d]pyrimidine

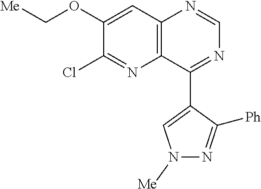 [SMILES: CCOc1cc2ncnc(-c3cn(C)nc3-c3ccccc3)c2nc1Cl]

Pd(PPh₃)₄ (235 mg, 0.204 mmol) and K₃PO₄ (864 mg, 4.08 mmol) were added to 4,6-dichloro-7-ethoxypyrido[3,2-d]pyrimidine (Preparation 230, 500 mg, 2.04 mmol) and 1-methyl-3-phenyl-4-(4,4,5,5-tetramethyl-1,3,2-dioxaborolan-2-yl)-1H-pyrazole (579 mg, 2.04 mmol) in dioxane/H₂O (16 mL/4 mL) at rt and the resulting mixture was heated to 80° C. for 2 h under N₂. The reaction mixture was diluted with EtOAc (200 mL), washed with water (3×100 mL) and saturated brine (100 mL). The combined organics were dried (Na₂SO₄) and evaporated to dryness in vacuo. The residue was purified by silica gel column chromatography (20:1 DCM/MeOH) to afford the title compound as an off-white solid (290 mg, 39%). LCMS: m/z=366 [M+H]⁺.

### Preparation 232: 6-chloro-7-ethoxy-4-(3-(4-fluorophenyl)-1-methyl-1H-pyrazol-4-yl)pyrido[3,2-d]pyrimidine

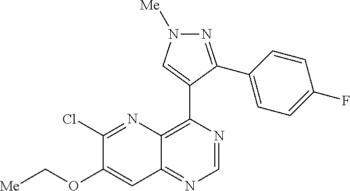 [SMILES: CCOc1cc2ncnc(-c3cn(C)nc3-c3ccc(F)cc3)c2nc1Cl]

The title compound was obtained as a yellow solid, 1 g, 32%, from 4,6-dichloro-7-ethoxypyrido[3,2-d]pyrimidine (Preparation 230) and 3-(4-fluorophenyl)-1-methyl-4-(4,4,5,5-tetramethyl-1,3,2-dioxaborolan-2-yl)-1H-pyrazole (Preparation 43) following a similar procedure to that described in Preparation 97. LCMS: m/z=385 [M+H]⁺.

### Preparation 233: 6-chloro-7-ethoxy-4-(3-(2-fluorophenyl)-1-methyl-1H-pyrazol-4-yl)pyrido[3,2-d]pyrimidine

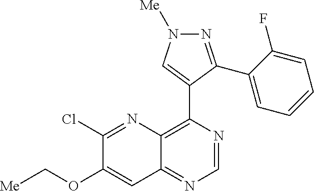 [SMILES: CCOc1cc2ncnc(-c3cn(C)nc3-c3ccccc3F)c2nc1Cl]

The title compound was obtained as a yellow solid, 500 mg, 20%, from 4,6-dichloro-7-ethoxypyrido[3,2-d]pyrimidine (Preparation 230) and 3-(2-fluorophenyl)-1-methyl-4-(4,4,5,5-tetramethyl-1,3,2-dioxaborolan-2-yl)-1H-pyrazole (Preparation 37) following a similar procedure to that described in Preparation 231. LCMS: m/z=384 [M+H]⁺.

### Preparation 234: 6-chloro-7-methoxy-4-(3-phenyl-1-(tetrahydro-2H-pyran-2-yl)-1H-pyrazol-4-yl)pyrido[3,2-d]pyrimidine

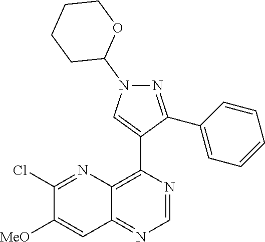 [SMILES: COc1cc2ncnc(-c3cn(C4CCCCO4)nc3-c3ccccc3)c2nc1Cl]

The title compound was obtained as a yellow solid, 4.2 g, 42% yield, from 4,6-dichloro-7-methoxypyrido[3,2-d]pyrimidine (Preparation 224) and 3-phenyl-1-(tetrahydro-2H-pyran-2-yl)-4-(4,4,5,5-tetramethyl-1,3,2-dioxaborolan-2-yl)-1H-pyrazole (Preparation 42) following a similar procedure to that described in Preparation 231. LCMS: m/z=442 [M+H]⁺.

### Preparation 235: 6-chloro-7-methoxy-4-(3-phenyl-1H-pyrazol-4-yl)pyrido[3,2-d]pyrimidine

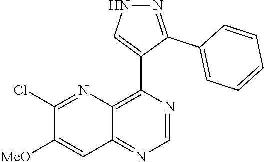 [SMILES: COc1cc2ncnc(-c3c[nH]nc3-c3ccccc3)c2nc1Cl]

A mixture of 6-chloro-7-methoxy-4-(3-phenyl-1-(tetrahydro-2H-pyran-2-yl)-1H-pyrazol-4-yl)pyrido[3,2-d]pyrimidine (Preparation 234, 2 g, 4.73 mmol) in TFA (10 mL) and DCM (30 mL) was stirred at rt for 3 h under N₂. The reaction mixture was diluted with EtOAc (120 mL) and washed with H₂O (60 mL). The combined organics were dried (Na₂SO₄) and evaporated to dryness in vacuo. The residue was purified by prep-TLC (50% DCM/EtOAc) to afford the title compound as a yellow solid (1.5 g, 94%). LCMS: m/z=338 [M+H]⁺.

### Preparation 236: 6-chloro-7-methoxy-4-(1-(methyl-d₃)-3-phenyl-1H-pyrazol-4-yl)pyrido[3,2-d]pyrimidine and 6-chloro-7-methoxy-4-(1-(methyl-d₃)-5-phenyl-1H-pyrazol-4-yl)pyrido[3,2-d]pyrimidine

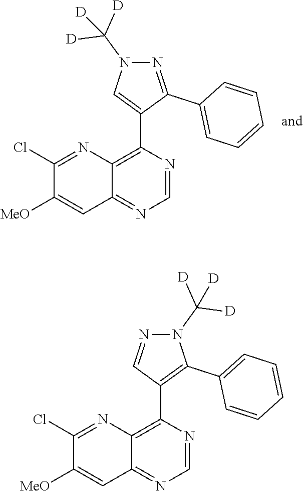 [SMILES: [2H]C([2H])([2H])n1cc(-c2ncnc3cc(OC)c(Cl)nc23)c(-c2ccccc2)n1 ; [2H]C([2H])([2H])n1ncc(-c2ncnc3cc(OC)c(Cl)nc23)c1-c1ccccc1]

A mixture of D₃I (1.96 g, 14.8 mmol), K₂CO₃ (2.04 g, 14.8 mmol) and 6-chloro-7-methoxy-4-(3-phenyl-1H-pyrazol-4-yl)pyrido[3,2-d]pyrimidine (Preparation 235, 1 g, 2.96 mmol) in DMF (10 mL) was stirred at rt for 3 h under N₂. The reaction mixture was diluted with EtOAc (120 mL) and washed with water (60 mL). The combined organics were dried (Na₂SO₄) and evaporated to dryness. The residue was purified by prep-TLC (2:1 DCM/EtOAc) to afford a mixture of 6-chloro-7-methoxy-4-(1-(methyl-d₃)-3-phenyl-1H-pyrazol-4-yl)pyrido[3,2-d]pyrimidine and 6-chloro-7-methoxy-4-(1-(methyl-d₃)-5-phenyl-1H-pyrazol-4-yl)pyrido[3,2-d]pyrimidine as a yellow solid (700 mg, 67%) which was not purified further. LCMS: m/z=355 [M+H]⁺.

### Preparation 237: 6-chloro-4-(3-(2-fluorophenyl)-1-methyl-1H-pyrazol-4-yl)-7-methoxypyrido[3,2-d]pyrimidine

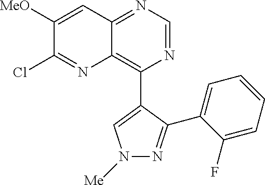 [SMILES: COc1cc2ncnc(-c3cn(C)nc3-c3ccccc3F)c2nc1Cl]

The title compound was obtained as a white solid, 150 mg, 47%, from 4,6-dichloro-7-methoxypyrido[3,2-d]pyrimidine (Preparation 224) and 3-(2-fluorophenyl)-1-methyl-4-(4,4,5,5-tetramethyl-1,3,2-dioxaborolan-2-yl)-1H-pyrazole (Preparation 37), following a similar procedure to that described in Preparation 231. LCMS: m/z=370 [M+H]⁺.

### Preparation 238: 6-chloro-4-(3-(4-fluorophenyl)-1-methyl-1H-pyrazol-4-yl)-7-methoxypyrido[3,2-d]pyrimidine

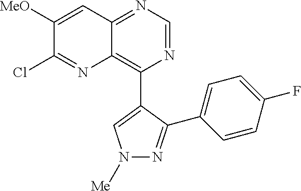 [SMILES: COc1cc2ncnc(-c3cn(C)nc3-c3ccc(F)cc3)c2nc1Cl]

The title compound was obtained as a yellow solid, 300 mg, 37%, from 4,6-dichloro-7-methoxypyrido[3,2-d]pyrimidine (Preparation 224, 500 mg, 2.17 mmol) and 3-(4-fluorophenyl)-1-methyl-4-(4,4,5,5-tetramethyl-1,3,2-dioxaborolan-2-yl)-1H-pyrazole (Preparation 43), following a similar procedure to that described in Preparation 231. LCMS: m/z=370 [M+H]⁺.

### Preparation 239: 6-chloro-4-(1-ethyl-3-phenyl-1H-pyrazol-4-yl)-7-methoxypyrido[3,2-d]pyrimidine

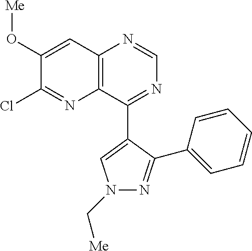 [SMILES: CCn1cc(-c2ncnc3cc(OC)c(Cl)nc23)c(-c2ccccc2)n1]

K₃PO₄ (5.0 g, 23.6 mmol) was added to Pd(dppf)Cl₂·DCM (1.41 g, 1.73 mmol), 4,6-dichloro-7-methoxypyrido[3,2-d]pyrimidine (Preparation 224, 5.0 g, 21.6 mmol) and 1-ethyl-3-phenyl-4-(4,4,5,5-tetramethyl-1,3,2-dioxaborolan-2-yl)-1H-pyrazole (Preparation 33, 5.79 g, 19.4 mmol) in dioxane/H₂O at rt and the reaction was stirred at 60° C. for 16 h. The reaction mixture was diluted with EtOAc (300 mL) and washed with brine (2×100 mL). The organic layer was dried over Na₂SO₄ and concentrated in vacuo. The residue was purified by column chromatography (SiO₂, 5:1 DCM/EtOAc) to afford the title compound as a yellow solid (1.6 g, 23%). LCMS m/z=366 [M+H]⁺.

### Preparation 240: 6-chloro-4-(1-(2,2-difluoroethyl)-3-phenyl-1H-pyrazol-4-yl)-7-methoxypyrido[3,2-d]pyrimidine

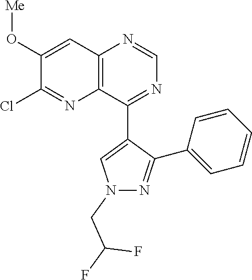 [SMILES: COc1cc2ncnc(-c3cn(CC(F)F)nc3-c3ccccc3)c2nc1Cl]

The title compound was obtained as a yellow solid, 852 mg, 24% yield, from 4,6-dichloro-7-methoxypyrido[3,2-d]pyrimidine (Preparation 224) and 1-(2,2-difluoroethyl)-3-phenyl-4-(4,4,5,5-tetramethyl-1,3,2-dioxaborolan-2-yl)-1H-pyrazole (Preparation 41), following the procedure described in Preparation 239. LCMS m/z=402 [M+H]⁺.

### Preparation 241: 5-(6-chloro-7-methoxypyrido[3,2-d]pyrimidin-4-yl)-2-methyl-4-phenylthiazole

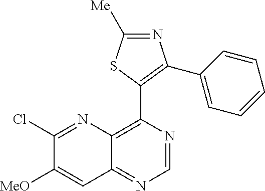 [SMILES: COc1cc2ncnc(-c3sc(C)nc3-c3ccccc3)c2nc1Cl]

The title compound was obtained as a yellow solid, 40 mg, 16% yield, from 4,6-dichloro-7-methoxypyrido[3,2-d]pyrimidine (Preparation 224) and, 2-methyl-4-phenyl-5-(4,4,5,5-tetramethyl-1,3,2-dioxaborolan-2-yl)-1,3-thiazole following a similar procedure to that described in preparation 231. LCMS: m/z=369 [M+H]⁺.

### Preparation 242: 6-chloro-7-methoxy-4-(1-methyl-3-phenyl-1H-pyrazol-4-yl)pyrido[3,2-d]pyrimidine

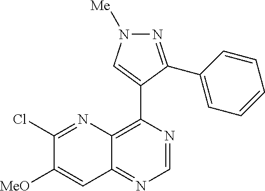 [SMILES: COc1cc2ncnc(-c3cn(C)nc3-c3ccccc3)c2nc1Cl]

K₃PO₄ (250 g, 1.18 mol) was added to Pd(dppf)Cl₂·DCM (70.5 g, 86.4 mmol), 4,6-dichloro-7-methoxypyrido[3,2-d]pyrimidine (Preparation 224, 250 g, 1.08 mol) and 1-methyl-3-phenyl-4-(4,4,5,5-tetramethyl-1,3,2-dioxaborolan-2-yl)-1H-pyrazole (276 g, 972 mmol) in dioxane/H₂O at rt and the resulting mixture stirred at 60° C. for 16 h. The reaction was diluted with EtOAc (3 L) and washed with brine (2×1 L). The combined organics were dried (Na₂SO₄) and evaporated to dryness in vacuo and the residue purified by a silica gel chromatography (5:1 DCM/EtOAc) to afford the title compound as a yellow solid (66 g, 17%. LCMS: m/z=352 [M+H]⁺.

### Preparation 243: 6-chloro-4-(3-(2,6-difluorophenyl)-1-methyl-1H-pyrazol-4-yl)-7-methoxypyrido[3,2-d]pyrimidine

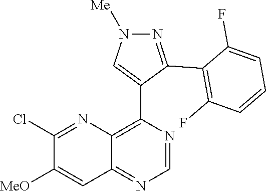 [SMILES: COc1cc2ncnc(-c3cn(C)nc3-c3c(F)cccc3F)c2nc1Cl]

A mixture of 4,6-dichloro-7-methoxypyrido[3,2-d]pyrimidine (Preparation 224, 600 mg, 2.60 mmol), 3-(2,6-difluorophenyl)-1-methyl-4-(4,4,5,5-tetramethyl-1,3,2-dioxaborolan-2-yl)-1H-pyrazole (Preparation 34, 915 mg, 2.86 mmol), Pd(PPh₃)₄ (300 mg, 0.260 mmol) and K₃PO₄ (1.10 g, 5.20 mmol) in dioxane (10 mL) and H₂O (2 mL) under N₂ and the mixture was stirred at 80° C. for 3 h. The reaction mixture was extracted with EtOAc and the combined organics were dried and evaporated to dryness in vacuo. The residue was purified by prep-TLC (15:1 DCM/MeOH) to afford the title compound as a white solid (200 mg, 20%). LCMS: m/z=388 [M+H]⁺.

### Preparation 244: 6-chloro-4-(1-(difluoromethyl)-3-phenyl-1H-pyrazol-4-yl)-7-methoxypyrido[3,2-d]pyrimidine

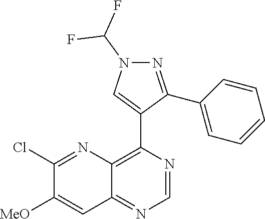 [SMILES: COc1cc2ncnc(-c3cn(C(F)F)nc3-c3ccccc3)c2nc1Cl]

A mixture of 6-chloro-7-methoxy-4-(3-phenyl-1H-pyrazol-4-yl)pyrido[3,2-d]pyrimidine (Preparation 235, 200 mg, 0.592 mmol), diethyl(bromodifluoromethyl)phosphonate (315 mg, 1.18 mmol), KF (68.5 mg, 1.18 mmol) and KI (195 mg, 1.18 mmol) in MeCN (10 mL) was stirred at 60° C. for 16 h. The reaction mixture was evaporated to dryness in vacuo and the residue purified by prep-TLC (30:1 DCM/MeOH) to afford the title compound as a yellow solid (60 mg, 26%). LCMS: m/z=487 [M+H]⁺.

### Preparation 245: 6-chloro-4-(1-cyclopropyl-3-phenyl-1H-pyrazol-4-yl)-7-methoxypyrido[3,2-d]pyrimidine

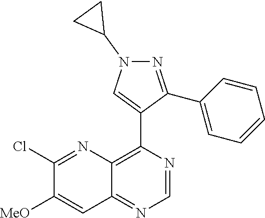 [SMILES: COc1cc2ncnc(-c3cn(C4CC4)nc3-c3ccccc3)c2nc1Cl]

A mixture of Cu(OAc)₂ (192 mg, 0.592 mmol), 2,2-bipyridine (92.4 mg, 0.592 mmol) and DCE (15 mL) was stirred at 80° C. for 0.5 h. 6-Chloro-7-methoxy-4-(3-phenyl-1H-pyrazol-4-yl)pyrido[3,2-d]pyrimidine (Preparation 235, 100 mg, 0.296 mmol), cyclopropylboronic acid (50.8 mg, 0.592 mmol) and Na₂CO₃ (62.7 mg, 0.592 mmol) were added and the resulting mixture stirred at 80° C. for 4 h under N₂. The reaction mixture was evaporated to dryness in vacuo and the residue was purified by prep-TLC (30:1 DCM/MeOH) to afford the title compound as a yellow solid (60 mg, 86%). LCMS: m/z=378 [M+H]⁺.

### Preparation 246:6-chloro-4-(1-(cyclopropylmethyl)-3-phenyl-1H-pyrazol-4-yl)-7-methoxypyrido[3,2-d]pyrimidine

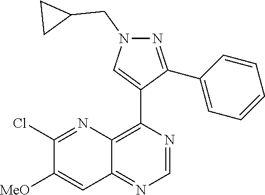 [SMILES: COc1cc2ncnc(-c3cn(CC4CC4)nc3-c3ccccc3)c2nc1Cl]

A mixture of 6-chloro-7-methoxy-4-(3-phenyl-1H-pyrazol-4-yl)pyrido[3,2-d]pyrimidine (Preparation 235, 200 mg, 0.592 mmol), (bromomethyl)cyclopropane (159 mg, 1.18 mmol) and Cs₂CO₃ (384 mg, 1.18 mmol) in MeCN (8 mL) was stirred at 60° C. for 6 h. The reaction mixture was evaporated to dryness in vacuo and the residue purified by prep-TLC (40:1 DCM/MeOH) to afford title compound as a yellow solid (80 mg, 35%). LCMS: m/z=392 [M+H]⁺.

### Preparation 247: 4-(1-(2-((tert-butyldimethylsilyl)oxy)ethyl)-3-phenyl-1H-pyrazol-4-yl)-6-chloro-7-methoxypyrido[3,2-d]pyrimidine

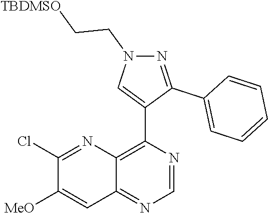 [SMILES: COc1cc2ncnc(-c3cn(CCO[Si](C)(C)C(C)(C)C)nc3-c3ccccc3)c2nc1Cl]

The title compound was prepared as a yellow solid (120 mg, 41%) from 6-chloro-7-methoxy-4-(3-phenyl-1H-pyrazol-4-yl)pyrido[3,2-d]pyrimidine (Preparation 235) and (2-bromoethoxy)(tert-butyl)dimethylsilane using an analogous methods to that described for Preparation 246. LCMS: m/z=496 [M+H]⁺.

### Preparation 248: N-(7-methoxy-4-(1-methyl-3-phenyl-1H-pyrazol-4-yl)quinazolin-6-yl)propionamide

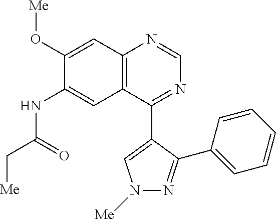 [SMILES: CCC(=O)Nc1cc2c(-c3cn(C)nc3-c3ccccc3)ncnc2cc1OC]

To a solution of 7-methoxy-4-(1-methyl-3-phenyl-1H-pyrazol-4-yl)quinazolin-6-amine (Preparation 190, 5 g, 15.0 mmol) and propanoic acid (1.66 g, 22.6 mmol) in THF (50 mL) were added T3P® (10 mL, 50% solution in EtOAc) and pyridine (1.18 g, 15.0 mmol). The reaction was stirred at rt for 2 h and quenched with water. The reaction mixture was extracted with EtOAc, the organic layers were dried over Na₂SO₄, then concentrated in vacuo. The residue was purified by column chromatography (SiO₂, 20:1 DCM/MeOH) to afford the title compound as a yellow solid (5.81 g, 99%). LCMS m/z=388 [M+H]⁺.

### Preparation 249: N-(7-hydroxy-4-(1-methyl-3-phenyl-1H-pyrazol-4-yl)quinazolin-6-yl)propionamide

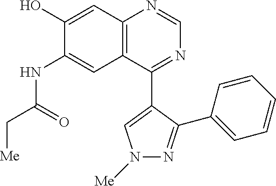 [SMILES: CCC(=O)Nc1cc2c(-c3cn(C)nc3-c3ccccc3)ncnc2cc1O]

To N-(7-methoxy-4-(1-methyl-3-phenyl-1H-pyrazol-4-yl)quinazolin-6-yl)propionamide (Preparation 248, 400 mg, 1.03 mmol) in pyridine (4 mL) was added pyridine-HCl (1.18 g, 10.3 mmol) and the reaction was stirred at 100° C. overnight. The reaction was concentrated in vacuo and adjusted to pH 8 with TEA. The residue was purified by prep-TLC (10:1 DCM/MeOH) to afford the title compound as yellow oil (200 mg, 52%). LCMS m/z=374 [M+H]⁺.

### Preparation 250: 4-(1-methyl-3-phenyl-1H-pyrazol-4-yl)-6-propanamidoquinazolin-7-yl trifluoromethanesulfonate

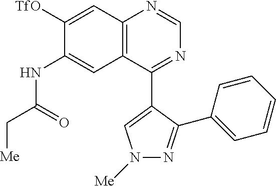 [SMILES: CCC(=O)Nc1cc2c(-c3cn(C)nc3-c3ccccc3)ncnc2cc1OS(=O)(=O)C(F)(F)F]

To N-(7-hydroxy-4-(1-methyl-3-phenyl-1H-pyrazol-4-yl)quinazolin-6-yl)propionamide (Preparation 249, 200 mg, 0.535 mmol) and TEA (226 mg, 0.802 mmol) in DCM (2 mL) was added Tf₂O (226 mg, 0.802 mmol) dropwise at 0° C. under N₂ and the reaction was stirred at rt for 2 h. The reaction was quenched with water/ice and extracted with DCM. The combined organics were concentrated in vacuo and the residue was purified by prep-TLC (5:1 DCM/MeOH) to afford the title compound as a yellow solid (100 mg, 37%). LCMS m/z=506 [M+H]⁺.

### Preparation 251: 4-(1-(2,2-difluoroethyl)-3-phenyl-1H-pyrazol-4-yl)-7-methoxyquinazolin-6-amine

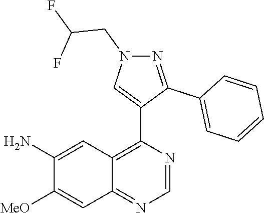 [SMILES: COc1cc2ncnc(-c3cn(CC(F)F)nc3-c3ccccc3)c2cc1N]

The title compound was prepared as a yellow solid (300 mg, 56%) from 4-bromo-7-methoxy-6-nitroquinazoline (Preparation 189) and 1-(2,2-difluoroethyl)-3-phenyl-4-(4,4,5,5-tetramethyl-1,3,2-dioxaborolan-2-yl)-1H-pyrazole (Preparation 41) using an analogous 2-part process as described for Preparation 190. LCMS: m/z=382 [M+H]⁺.

### Preparation 252: 4-(1-ethyl-3-phenyl-1H-pyrazol-4-yl)-7-methoxyquinazolin-6-amine

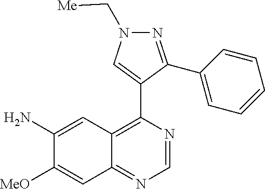 [SMILES: CCn1cc(-c2ncnc3cc(OC)c(N)cc23)c(-c2ccccc2)n1]

The title compound was prepared as a white solid (120 mg) from 4-bromo-7-methoxy-6-nitroquinazoline (Preparation 189) and 1-ethyl-3-phenyl-4-(4,4,5,5-tetramethyl-1,3,2-dioxaborolan-2-yl)-1H-pyrazole (Preparation 33) using an analogous 2-part process as described for Preparation 190. LCMS: m/z=346 [M+H]⁺.

### Preparation 253: 4-(3-(2-fluorophenyl)-1-methyl-1H-pyrazol-4-yl)-7-methoxyquinazolin-6-amine

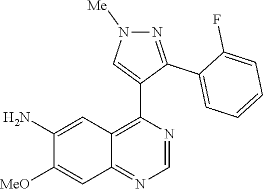 [SMILES: COc1cc2ncnc(-c3cn(C)nc3-c3ccccc3F)c2cc1N]

The title compound was prepared as a white solid (80 mg, 77%) from 7-methoxy-6-nitro-3,4-dihydroquinazolin-4-one and 3-(2-fluorophenyl)-1-methyl-4-(4,4,5,5-tetramethyl-1,3,2-dioxaborolan-2-yl)-1H-pyrazole (Preparation 37) using an analogous 2-part method as described for Preparation 190. LCMS: m/z=350 [M+H]⁺.

### Preparation 254: 4-(3-(4-fluorophenyl)-1-methyl-1H-pyrazol-4-yl)-7-methoxyquinazolin-6-amine

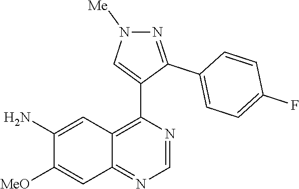 [SMILES: COc1cc2ncnc(-c3cn(C)nc3-c3ccc(F)cc3)c2cc1N]

The title compound was prepared as a yellow solid (100 mg, 54%) from 7-methoxy-6-nitro-3,4-dihydroquinazolin-4-one and 3-(4-fluorophenyl)-1-methyl-4-(4,4,5,5-tetramethyl-1,3,2-dioxaborolan-2-yl)-1H-pyrazole (Preparation 43) using an analogous 2-part method as described for Preparation 190. LCMS: m/z=380 [M+H]⁺.

### Preparation 255: 4-(1-ethyl-3-(4-fluorophenyl)-1H-pyrazol-4-yl)-7-methoxyquinazolin-6-amine

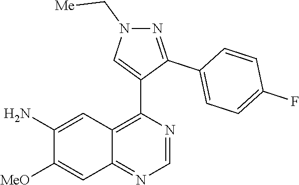 [SMILES: CCn1cc(-c2ncnc3cc(OC)c(N)cc23)c(-c2ccc(F)cc2)n1]

To a mixture of 4-(1-ethyl-3-(4-fluorophenyl)-1H-pyrazol-4-yl)-7-methoxy-6-nitroquinazoline (Preparation 205, 120 mg, 0.305 mmol) in EtOH (5 mL) and H₂O (0.5 mL) was added Fe (341 mg, 6.10 mmol) and HCl (0.2 mL) and the reaction mixture was stirred at 80° C. for 3 h. The cooled reaction mixture was filtered and the filtrate was evaporated to dryness. The residue was purified by prep-TLC (20:1 DCM/MeOH) to afford the title compound as a yellow solid (80 mg, 73%). LCMS: m/z=364 [M+H]⁺.

### Preparation 256: 5-fluoro-7-methoxy-4-(1-methyl-3-phenyl-1H-pyrazol-4-yl)quinazolin-6-amine

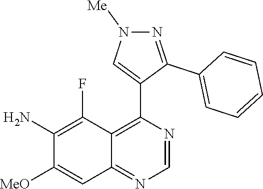 [SMILES: COc1cc2ncnc(-c3cn(C)nc3-c3ccccc3)c2c(F)c1N]

Selectfluor (266 mg, 0.754 mmol) was added to 7-methoxy-4-(1-methyl-3-phenyl-1H-pyrazol-4-yl)quinazolin-6-amine (Preparation 190, 250 mg, 0.754 mmol) in MeCN (15 mL) at rt and the mixture stirred at rt for 2 h. The mixture was diluted with EtOAc (100 mL) and washed with brine (2×50 mL). The combined organics were dried (Na₂SO₄) and evaporated to dryness in vacuo. The residue was purified by column chromatography (SiO₂, 15:1 DCM/MeOH) to afford the title compound as a yellow solid (180 mg, 37%). LCMS: m/z=350 [M+H]⁺.

### Preparation 257: tert-butyl 1-((7-methoxy-4-(1-methyl-3-phenyl-1H-pyrazol-4-yl)quinazolin-6-yl)carbamoyl)-3-azabicyclo[3.1.0]hexane-3-carboxylate

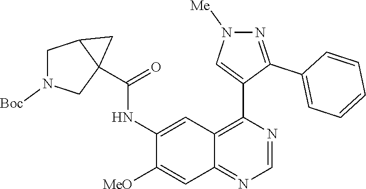 [SMILES: COc1cc2ncnc(-c3cn(C)nc3-c3ccccc3)c2cc1NC(=O)C12CC1CN(C(=O)OC(C)(C)C)C2]

The title compound was prepared as a yellow solid (310 mg, 95%) from 7-methoxy-4-(1-methyl-3-phenyl-1H-pyrazol-4-yl)quinazolin-6-amine (Preparation 190) and tert-butyl 1-carbamoyl-3-azabicyclo[3.1.0]hexane-3-carboxylate (Preparation 65) using an analogous method to that described for Example 205 LCMS: m/z=541 [M+H]⁺.

### Preparation 258: tert-butyl 1-((4-(3-(2-fluorophenyl)-1-methyl-1H-pyrazol-4-yl)-7-methoxypyrido[3,2-d]pyrimidin-6-yl)carbamoyl)-3-azabicyclo[3.1.0]hexane-3-carboxylate

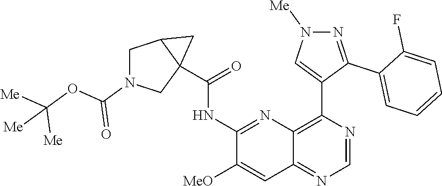 [SMILES: COc1cc2ncnc(-c3cn(C)nc3-c3ccccc3F)c2nc1NC(=O)C12CC1CN(C(=O)OC(C)(C)C)C2]

A mixture of BINAP Pd G3 (35.2 mg, 0.0378 mmol), Cs₂CO₃ (184 mg, 0.567 mmol), 6-chloro-4-(3-(2-fluorophenyl)-1-methyl-1H-pyrazol-4-yl)-7-methoxypyrido[3,2-d]pyrimidine (Preparation 237, 140 mg, 0.378 mmol) and tert-butyl 1-carbamoyl-3-azabicyclo[3.1.0]hexane-3-carboxylate (Preparation 65, 128 mg, 0.567 mmol) in dioxane (10 mL) was stirred at 100° C. for 3 h under N₂. The reaction mixture was diluted with water, extracted with EtOAc (3×50 mL) and the combined extracts were dried (Na₂SO₄) and evaporated to dryness in vacuo. The residue was purified by prep-TLC (25:1 DCM/MeOH) to afford the title compound as a yellow solid (100 mg, 47%). LCMS: m/z=560 [M+H]⁺.

### Preparation 259: tert-butyl 1-((7-ethoxy-4-(3-(4-fluorophenyl)-1-methyl-1H-pyrazol-4-yl)pyrido[3,2-d]pyrimidin-6-yl)carbamoyl)-3-azabicyclo[3.1.0]hexane-3-carboxylate

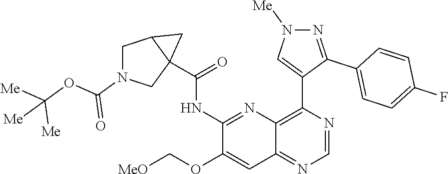 [SMILES: COCOc1cc2ncnc(-c3cn(C)nc3-c3ccc(F)cc3)c2nc1NC(=O)C12CC1CN(C(=O)OC(C)(C)C)C2]

The title compound was prepared as a yellow solid (320 mg, 71%) from 6-chloro-7-ethoxy-4-(3-(4-fluorophenyl)-1-methyl-1H-pyrazol-4-yl)pyrido[3,2-d]pyrimidine (Preparation 232) and tert-butyl 1-carbamoyl-3-azabicyclo[3.1.0]hexane-3-carboxylate (Preparation 65) using an analogous method to that described for Preparation 258. LCMS: m/z=574 [M+H]⁺.

### Preparation 260: tert-butyl 6,6-difluoro-1-((7-methoxy-4-(1-methyl-3-phenyl-1H-pyrazol-4-yl)pyrido[3,2-d]pyrimidin-6-yl)carbamoyl)-3-azabicyclo[3.1.0]hexane-3-carboxylate

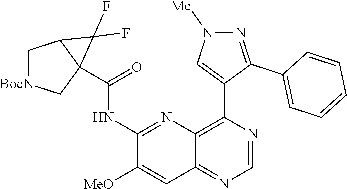 [SMILES: COc1cc2ncnc(-c3cn(C)nc3-c3ccccc3)c2nc1NC(=O)C12CN(C(=O)OC(C)(C)C)CC1C2(F)F]

A mixture of 6-chloro-7-methoxy-4-(1-methyl-3-phenyl-1H-pyrazol-4-yl)pyrido[3,2-d]pyrimidine (Preparation 242, 110 mg, 0.312 mmol), tert-butyl 1-carbamoyl-6,6-difluoro-3-azabicyclo[3.1.0]hexane-3-carboxylate (Preparation 73, 81.8 mg, 0.312 mmol), Pd₂(dba); (5 mg, 5.46 μmol), XantPhos (180 mg, 0.312 mmol) and Cs₂CO₃ (101 mg, 0.312 mmol) in toluene (15 mL) was stirred at 100° C. for 3 h. The reaction mixture was evaporated to dryness in vacuo and the residue purified by prep-TLC (30:1 DCM/MeOH) to afford the title compound as a yellow solid (70 mg, 39%). LCMS: m/z=578 [M+H]⁺.

### Preparation 261: N-(4-(1-(2-((tert-butyldimethylsilyl)oxy)ethyl)-3-phenyl-1H-pyrazol-4-yl)-7-methoxypyrido[3,2-d]pyrimidin-6-yl)bicyclo[1.1.1]pentane-1-carboxamide

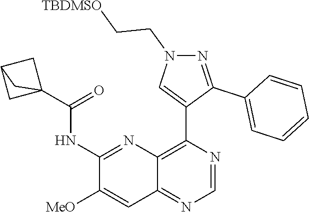 [SMILES: COc1cc2ncnc(-c3cn(CCO[Si](C)(C)C(C)(C)C)nc3-c3ccccc3)c2nc1NC(=O)C12CC(C1)C2]

The title compound was prepared as a yellow solid (80 mg, 58%) from 4-(1-(2-((tert-butyldimethylsilyl)oxy)ethyl)-3-phenyl-1H-pyrazol-4-yl)-6-chloro-7-methoxypyrido[3,2-d]pyrimidine (Preparation 247) and bicyclo[1.1.1]pentane-1-carboxamide (Preparation 70) using an analogous method to that described for Preparation 260. LCMS: m/z=571 [M+H]⁺.

### Preparation 262: N-(4-(1-(2-((tert-butyldimethylsilyl)oxy)ethyl)-3-phenyl-1H-pyrazol-4-yl)-7-methoxypyrido[3,2-d]pyrimidin-6-yl)-1-(trifluoromethyl)-1H-pyrazole-4-carboxamide

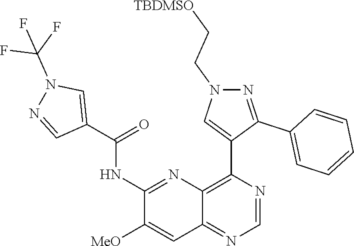 [SMILES: COc1cc2ncnc(-c3cn(CCO[Si](C)(C)C(C)(C)C)nc3-c3ccccc3)c2nc1NC(=O)c1cnn(C(F)(F)F)c1]

The title compound was prepared as a yellow solid (60 mg, 47%) from 4-(1-(2-((tert-butyldimethyl silyl)oxy)ethyl)-3-phenyl-1H-pyrazol-4-yl)-6-chloro-7-methoxypyrido[3,2-d]pyrimidine (Preparation 247) and 1-(trifluoromethyl)-1H-pyrazole-4-carboxamide (Preparation 76) using an analogous method to that described for Preparation 260. LCMS: m/z=639 [M+H]⁺.

### Preparation 263: tert-butyl (1R,3S,5R)-3-((7-methoxy-4-(1-methyl-3-phenyl-1H-pyrazol-4-yl)pyrido[3,2-d]pyrimidin-6-yl)carbamoyl)-2-azabicyclo[3.1.0]hexane-2-carboxylate

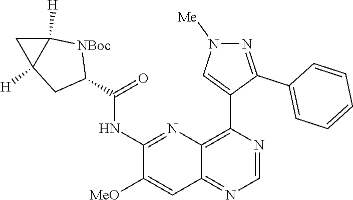 [SMILES: COc1cc2ncnc(-c3cn(C)nc3-c3ccccc3)c2nc1NC(=O)[C@@H]1C[C@H]2C[C@H]2N1C(=O)OC(C)(C)C]

The title compound was prepared as a yellow solid (100 mg, 43%) from tert-butyl (1R,3S,5R)-3-carbamoyl-2-azabicyclo[3.1.0]hexane-2-carboxylate (Preparation 74) and 6-chloro-7-methoxy-4-(1-methyl-3-phenyl-1H-pyrazol-4-yl)pyrido[3,2-d]pyrimidine (Preparation 242) using an analogous method to that described for Preparation 260. LCMS: m/z=542 [M+H]⁺.

### Preparation 264: tert-butyl (1R,3R,5R)-3-((7-methoxy-4-(1-methyl-3-phenyl-1H-pyrazol-4-yl)pyrido[3,2-d]pyrimidin-6-yl)carbamoyl)-2-azabicyclo[3.1.0]hexane-2-carboxylate

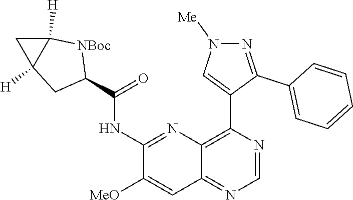 [SMILES: COc1cc2ncnc(-c3cn(C)nc3-c3ccccc3)c2nc1NC(=O)[C@H]1C[C@H]2C[C@H]2N1C(=O)OC(C)(C)C]

The title compound was prepared as a yellow solid (100 mg, 43%) from tert-butyl (1R,3R,5R)-3-carbamoyl-2-azabicyclo[3.1.0]hexane-2-carboxylate (Preparation 75) and 6-chloro-7-methoxy-4-(1-methyl-3-phenyl-1H-pyrazol-4-yl)pyrido[3,2-d]pyrimidine (Preparation 242) using an analogous method to that described for Preparation 260. LCMS: m/z=542 [M+H]⁺.

### Preparation 265: 7-(1-methyl-1H-pyrazol-4-yl)quinazolin-4 (3H)-one

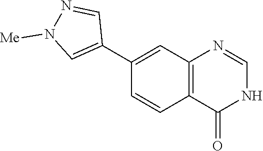 [SMILES: Cn1cc(-c2ccc3c(=O)[nH]cnc3c2)cn1]

7-Bromoquinazolin-4 (3H)-one (3.00 g, 13.3 mmol), 1-methyl-4-(4,4,5,5-tetramethyl-1,3,2-dioxaborolan-2-yl) pyrazole (3.33 g, 16.0 mmol), KHCO₃ (3.71 mg, 26.7 mmol) and Pd(dppf)Cl₂ (975 mg, 1.33 mmol) in dioxane (100 mL) and H₂O (20 mL) were placed into a round-bottom flask under N₂ and the resulting solution was stirred at 100° C. for 1 h. The reaction mixture was diluted with H₂O (50 mL), the solids were removed by filtration and the resulting solution was extracted with EtOAc (3×50 mL). The organic layer was concentrated in vacuo. The residue was purified by column chromatography (SiO₂, 20:1 DCM/MeOH) to afford the title compound as a light yellow solid (1.70 g, 56%). LCMS m/z=227 [M+H]⁺

### Preparation 266: 4-chloro-7-(1-methyl-1H-pyrazol-4-yl)quinazoline

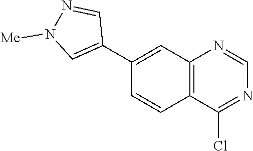 [SMILES: Cn1cc(-c2ccc3c(Cl)ncnc3c2)cn1]

7-(1-Methyl-1H-pyrazol-4-yl)quinazolin-4 (3H)-one (Preparation 265, 1.70 mg, 7.51 mmol), POCl₃ (10.0 mL) and DIPEA (2.91 g, 22.5 mmol) were placed into a 40-mL pressure tank reactor under N₂ and the resulting solution was stirred at 100° C. for 2 h. The mixture was concentrated under vacuum and the residue diluted with water (10 mL). The resulting solution was extracted with DCM (3×10 mL) and the combined organic layer was concentrated in vacuo to afford the title compound as a light yellow solid (1.30 g, 71%). LCMS m/z=245 [M+H]⁺.

### Preparation 267: 7-(1-methyl-1H-pyrazol-4-yl)-4-(3-phenyl-1-(tetrahydro-2H-pyran-2-yl)-1H-pyrazol-4-yl)quinazoline

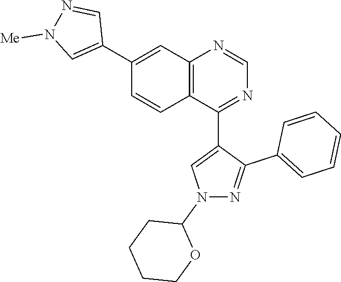 [SMILES: Cn1cc(-c2ccc3c(-c4cn(C5CCCCO5)nc4-c4ccccc4)ncnc3c2)cn1]

3-Phenyl-1-(tetrahydro-2H-pyran-2-yl)-4-(4,4,5,5-tetramethyl-1,3,2-dioxaborolan-2-yl)-1H-pyrazole (Preparation 42, 289 mg, 0.816 mmol), 4-chloro-7-(1-methyl-1H-pyrazol-4-yl)quinazoline (Preparation 266, 100 mg, 0.408 mmol) in DMSO (1 mL), Pd(PPh₃)₄ (47.1 mg, 0.0408 mmol) and K₃PO₄ (173 mg, 0.816 mmol) were placed into a 8-mL sealed tube. The reaction mixture was stirred at 100° C. for 2 h. The resulting solution was extracted with EtOAc (3×10 mL). The organic phase was concentrated under reduced pressure. The residue was purified by prep-TLC (20:1 DCM/MeOH) to afford the title compound as a white solid (65 mg, 36%). LCMS m/z=437 [M+H]⁺.

### Preparation 268: 4-(3-(2-fluorophenyl)-1-(tetrahydro-2H-pyran-2-yl)-1H-pyrazol-4-yl)-7-(1-methyl-1H-pyrazol-4-yl)quinazoline

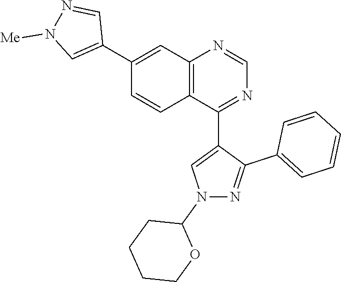 [SMILES: Cn1cc(-c2ccc3c(-c4cn(C5CCCCO5)nc4-c4ccccc4)ncnc3c2)cn1]

The title compound was obtained as a light yellow solid, 80 mg, 54% yield from 3-(2-fluorophenyl)-1-(tetrahydro-2H-pyran-2-yl)-4-(4,4,5,5-tetramethyl-1,3,2-dioxaborolan-2-yl)-1H-pyrazole (Preparation 46) and 4-chloro-7-(1-methyl-1H-pyrazol-4-yl)quinazoline (Preparation 266), following the procedure described in Preparation 267. LCMS m/z=455 [M+H]⁺.

### Preparation 269: 4-(3-(2,5-difluorophenyl)-1-(tetrahydro-2H-pyran-2-yl)-1H-pyrazol-4-yl)-7-(1-methyl-1H-pyrazol-4-yl)quinazoline

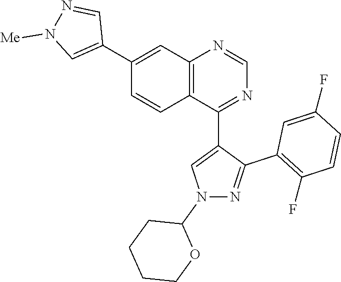 [SMILES: Cn1cc(-c2ccc3c(-c4cn(C5CCCCO5)nc4-c4cc(F)ccc4F)ncnc3c2)cn1]

The title compound was obtained as a white solid, 60 mg, from 4-chloro-7-(1-methyl-1H-pyrazol-4-yl)quinazoline (Preparation 266) and 3-(2,5-difluorophenyl)-1-(tetrahydro-2H-pyran-2-yl)-4-(4,4,5,5-tetramethyl-1,3,2-dioxaborolan-2-yl)-1H-pyrazole (Preparation 48) following a similar procedure to that described in Preparation 267.

### Preparation 270: 4-chloro-7-(1-(1-methylpiperidin-4-yl)-1H-pyrazol-4-yl)quinazoline

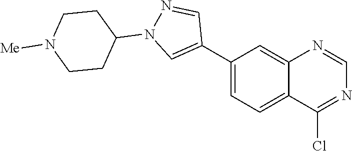 [SMILES: CN1CCC(n2cc(-c3ccc4c(Cl)ncnc4c3)cn2)CC1]

7-Bromo-4-chloroquinazoline (100 mg, 0.410 mmol) in DMF (1.2 mL), 1-methyl-4-(4-(4,4,5,5-tetramethyl-1,3,2-dioxaborolan-2-yl)-1H-pyrazol-1-yl) piperidine (238 mg, 0.820 mmol), Pd(dppf)Cl₂·DCM (33.4 mg, 0.041 mmol) and K₂CO₃ (113 mg, 0.820 mmol) were place into a 20-mL sealed tube. The reaction mixture was stirred at 100° C. for 16 h. The resulting solution was concentrated in vacuo. The residue was purified by prep-TLC (20:1 DCM/MeOH) to afford the title compound as a white solid (80 mg, 60%).

### Preparation 271: 7-(1-(1-methylpiperidin-4-yl)-1H-pyrazol-4-yl)-4-(3-phenyl-1-(tetrahydro-2H-pyran-2-yl)-1H-pyrazol-4-yl)quinazoline

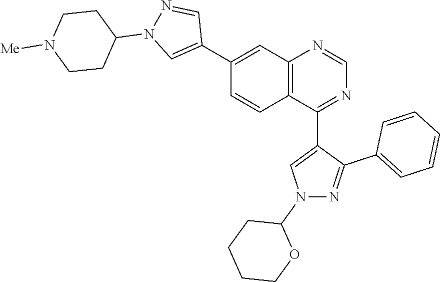 [SMILES: CN1CCC(n2cc(-c3ccc4c(-c5cn(C6CCCCO6)nc5-c5ccccc5)ncnc4c3)cn2)CC1]

The title compound was obtained as a yellow solid, 25 mg, 16% yield, from 3-phenyl-1-(tetrahydro-2H-pyran-2-yl)-4-(4,4,5,5-tetramethyl-1,3,2-dioxaborolan-2-yl)-1H-pyrazole (Preparation 42) and 4-chloro-7-(1-(1-methylpiperidin-4-yl)-1H-pyrazol-4-yl)quinazoline (Preparation 270) following a similar procedure to that described in Preparation 267. LCMS m/z=437 [M+H]⁺.

### Preparation 272: 5-fluoro-7-(1-methyl-1H-pyrazol-4-yl)quinazolin-4 (3H)-one

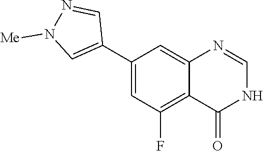 [SMILES: Cn1cc(-c2cc(F)c3c(=O)[nH]cnc3c2)cn1]

The title compound was obtained as a white solid, 400 mg, 80% yield, from 1-methyl-4-(4,4,5,5-tetramethyl-1,3,2-dioxaborolan-2-yl)-1H-pyrazole and 7-bromo-5-fluoroquinazolin-4 (3H)-one following a similar procedure to that described in Preparation 265. LCMS m/z=245 [M+H]⁺.

### Preparation 273: 4-chloro-5-fluoro-7-(1-methyl-1H-pyrazol-4-yl)quinazoline

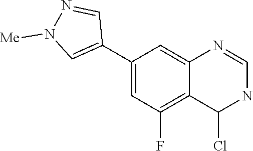 [SMILES: Cn1cc(-c2cc(F)c3c(c2)N=CNC3Cl)cn1]

The title compound was obtained as a white solid, 200 mg, 47% yield, from 5-fluoro-7-(1-methyl-1H-pyrazol-4-yl)quinazolin-4 (3H)-one (Preparation 272), following a similar procedure to that described in Preparation 266. LCMS m/z=263 [M+H]⁺.

### Preparation 274: 6-(benzyloxy)-7-bromo-4-(1-(2,2-difluoroethyl)-3-phenyl-1H-pyrazol-4-yl)quinazoline

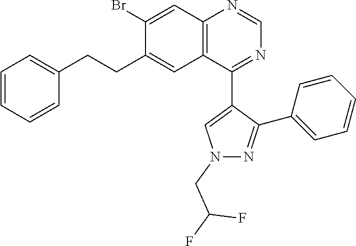 [SMILES: FC(F)Cn1cc(-c2ncnc3cc(Br)c(CCc4ccccc4)cc23)c(-c2ccccc2)n1]

To a mixture of 6-(benzyloxy)-7-bromo-4-chloroquinazoline (Preparation 150, 1 g, 2.86 mmol) and 1-(2,2-difluoroethyl)-3-phenyl-4-(4,4,5,5-tetramethyl-1,3,2-dioxaborolan-2-yl)-1H-pyrazole (Preparation 41, 955 mg, 2.86 mmol) in dioxane (20 mL) and H₂O (5 mL) was added Pd(dppf)Cl₂ (209 mg, 0.286 mmol) and K₂CO₃ (789 mg, 5.72 mmol) under N₂ and the reaction was stirred at 80° C. for 4 h. The mixture was diluted with water (40 mL) and extracted with EtOAc (2×40 mL). The combined organic layers were washed with brine (20 mL), dried over Na₂SO₄ and concentrated under reduced pressure. The crude product was purified by prep-TLC (10:1 DCM/MeOH) to afford the title compound as a yellow solid (700 mg, 74%). LCMS m/z=521 [M+H]⁺.

### Preparation 275: 6-(benzyloxy)-4-(1-(2,2-difluoroethyl)-3-phenyl-1H-pyrazol-4-yl)-7-(1-methyl-1H-pyrazol-4-yl)quinazoline

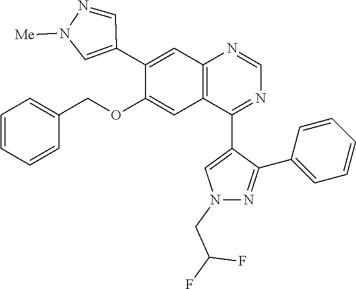 [SMILES: Cn1cc(-c2cc3ncnc(-c4cn(CC(F)F)nc4-c4ccccc4)c3cc2OCc2ccccc2)cn1]

The title compound was obtained as a white solid, 550 mg, 79% yield, from 6-(benzyloxy)-7-bromo-4-(1-(2,2-difluoroethyl)-3-phenyl-1H-pyrazol-4-yl)quinazoline (Preparation 274) and 1-methyl-4-(4,4,5,5-tetramethyl-1,3,2-dioxaborolan-2-yl)-1H-pyrazole, following a similar procedure to that described in Preparation 274. LCMS m/z=523 [M+H]⁺.

### Preparation 276: 4-(1-(2,2-difluoroethyl)-3-phenyl-1H-pyrazol-4-yl)-7-(1-methyl-1H-pyrazol-4-yl)quinazolin-6-ol

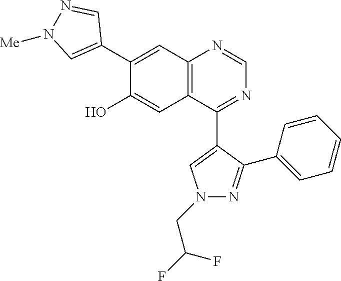 [SMILES: Cn1cc(-c2cc3ncnc(-c4cn(CC(F)F)nc4-c4ccccc4)c3cc2O)cn1]

To a mixture of 6-(benzyloxy)-4-(1-(2,2-difluoroethyl)-3-phenyl-1H-pyrazol-4-yl)-7-(1-methyl-1H-pyrazol-4-yl)quinazoline (Preparation 275, 550 mg, 1.05 mmol) in MeOH (10 mL) was added Pd/C (150 mg) and the reaction was stirred at 25° C. for 4 h under a H₂ atmosphere. The mixture was filtered and the filtrate evaporated to dryness to afford the title compound as a yellow solid (400 mg, 88.1%).

### Preparation 277: 4-(1-(2,2-difluoroethyl)-3-phenyl-1H-pyrazol-4-yl)-7-(1-methyl-1H-pyrazol-4-yl)quinazolin-6-yl trifluoromethanesulfonate

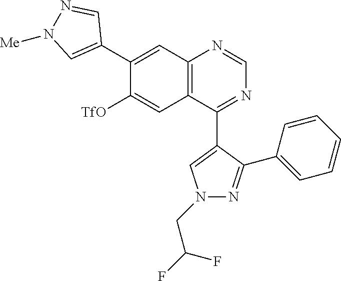 [SMILES: Cn1cc(-c2cc3ncnc(-c4cn(CC(F)F)nc4-c4ccccc4)c3cc2OS(=O)(=O)C(F)(F)F)cn1]

A solution of 4-(1-(2,2-difluoroethyl)-3-phenyl-1H-pyrazol-4-yl)-7-(1-methyl-1H-pyrazol-4-yl)quinazolin-6-ol (Preparation 276, 400 mg, 0.925 mmol) in DCM (10 mL) and TEA (233 mg, 2.31 mmol) was cooled to −50° C. Tf₂O (467 mg, 1.84 mmol) was added dropwise and the reaction was stirred for 1 h. The reaction was quenched with water and extracted with DCM (2×50 mL). The combined organic layers were washed with brine (50 mL), dried over Na₂SO₄ and concentrated under reduced pressure. The crude product was purified by column chromatography (SiO₂, 1:1 DCM/EtOAc) to afford the title compound as a yellow solid (500 mg, 96%). LCMS m/z=565 [M+H]⁺.

### Preparation 278: 4-(1-(2,2-difluoroethyl)-3-phenyl-1H-pyrazol-4-yl)-6-(1-ethoxyvinyl)-7-(1-methyl-1H-pyrazol-4-yl)quinazoline

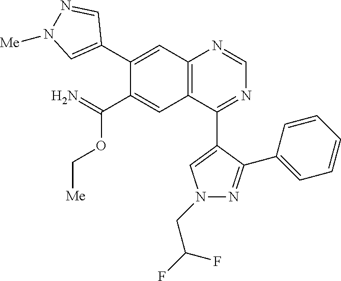 [SMILES: CCOC(=N)c1cc2c(-c3cn(CC(F)F)nc3-c3ccccc3)ncnc2cc1-c1cnn(C)c1]

To a solution of 4-(1-(2,2-difluoroethyl)-3-phenyl-1H-pyrazol-4-yl)-7-(1-methyl-1H-pyrazol-4-yl)quinazolin-6-yl trifluoromethanesulfonate (Preparation 277, 500 mg, 0.886 mmol) in dioxane/H₂O was added tributyl(1-ethoxyvinyl) stannane (319 mg, 0.885 mmol) K₂CO₃ (244 mg, 1.77 mmol) and Pd(PPh₃)₂Cl₂ (57.5 mg, 0.089 mmol) under N₂. The reaction mixture was stirred at 100° C. for 4 h, then cooled to rt, diluted with water (25 mL) and extracted with EtOAc (2×40 mL). The combined organic layers were washed with brine (20 mL), then dried over Na₂SO₄ and concentrated under reduced pressure. The crude product was purified by column chromatography (SiO₂, 1:1 PE/EtOAc) to afford the title compound as a yellow solid (400 mg, 93%). LCMS m/z=487 [M+H]⁺.

### Preparation 279: 1-(4-(1-(2,2-difluoroethyl)-3-phenyl-1H-pyrazol-4-yl)-7-(1-methyl-1H-pyrazol-4-yl)quinazolin-6-yl)ethan-1-one

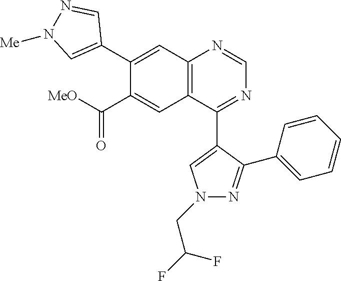 [SMILES: COC(=O)c1cc2c(-c3cn(CC(F)F)nc3-c3ccccc3)ncnc2cc1-c1cnn(C)c1]

A solution of 4-(1-(2,2-difluoroethyl)-3-phenyl-1H-pyrazol-4-yl)-6-(1-ethoxyvinyl)-7-(1-methyl-1H-pyrazol-4-yl)quinazoline (Preparation 278, 400 mg, 0.822 mmol) in TFA (3 mL) and DCM (9 mL) was stirred at 25° C. for 1 h. The reaction mixture was concentrated under reduced pressure to yield the title compound as yellow solid (220 mg 58%) which was used without further purification in the next step. LCMS m/z=489 [M+H]⁺.

### Preparation 280: 7-bromo-4-chloro-6-methoxyquinazoline

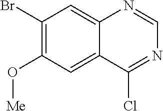 [SMILES: COc1cc2c(Cl)ncnc2cc1Br]

TEA (479 mg, 4.70 mmol) was added to POCl₃ (599 mg, 3.92 mmol) and 7-bromo-6-methoxy quinazolin-4 (3H)-one (200 mg, 0.784 mmol) in MeCN (1 mL) at rt. The reaction mixture was heated to 80° C. for 2 h. The resulting solution was added to ice-water followed by Na₂CO₃ (1 M) to adjust to pH 8. The solution was extracted with EtOAc and concentrated under reduced pressure. The crude product was purified by column chromatography (SiO₂, 100% DCM) to afford the title compound as a yellow solid (60 mg, 28%). LCMS m/z=273 [M+H]⁺.

### Preparation 281: 7-bromo-6-methoxy-4-(1-methyl-3-phenyl-1H-pyrazol-4-yl)quinazoline

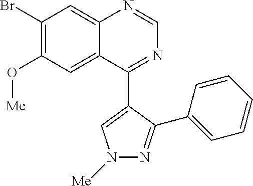 [SMILES: COc1cc2c(-c3cn(C)nc3-c3ccccc3)ncnc2cc1Br]

The title compound was obtained as a yellow solid, 40 mg, 46%, from 7-bromo-4-chloro-6-methoxy quinazoline (Preparation 280) and 1-methyl-3-phenyl-4-(4,4,5,5-tetramethyl-1,3,2-dioxaborolan-2-yl)-1H-pyrazole, following the procedure described in Preparation 231. LCMS m/z=395 [M+H]⁺.

### Preparation 282: 7-(1-methyl-1H-pyrazol-3-yl)-6-nitroquinazolin-4 (3H)-one

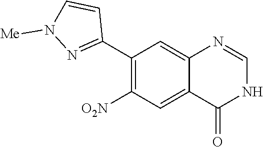 [SMILES: Cn1ccc(-c2cc3nc[nH]c(=O)c3cc2[N+](=O)[O-])n1]

To a solution of 7-bromo-6-nitro-3,4-dihydroquinazolin-4-one (500 mg, 1.85 mmol) in dioxane and water was added 1-methyl-3-(4,4,5,5-tetramethyl-1,3-dioxolan-2-yl)-1H-pyrazole (389 mg, 1.85 mmol), K₂CO₃ (511 mg, 3.70 mmol) and Pd(dppf)Cl₂ (270 mg, 0.370 mmol) and the reaction was stirred at 100° C. for 2 h. The mixture was extracted with EtOAc (20 mL×3) and washed with brine (10 mL). The organic layer was dried over Na₂SO₄ and concentrated under vacuum. The residue was purified by prep TLC (DCM:MeOH=10:1) to give the title compound, 400 mg, 80% as a yellow solid. LCMS: m/z=272 [M+H]⁺.

### Preparation 283: 4-bromo-7-(1-methyl-1H-pyrazol-3-yl)-6-nitroquinazoline

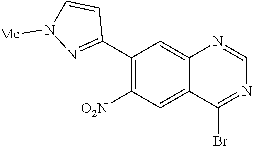 [SMILES: Cn1ccc(-c2cc3ncnc(Br)c3cc2[N+](=O)[O-])n1]

A solution of 7-(1-methyl-1H-pyrazol-3-yl)-6-nitroquinazolin-4 (3H)-one (Preparation 282, 400 mg, 1.47 mmol), DIPEA (400 mg, 1.47 mmol) and POBr₃ (400 mg, 2.20 mmol) in MeCN (5 mL) was stirred at 80° C. for 2 h. The mixture was poured into ice-NaHCO₃, extracted with EtOAc (3×20 mL) and washed with brine (10 mL). The organic layer was dried over Na₂SO₄ and concentrated under reduced pressure. The residue was purified by prep-TLC (10:1 DCM/MeOH) to afford the title compound as a yellow solid (280 mg, 57%). LCMS m/z=334 [M+H]⁺.

### Preparation 284: 4-(3-(2,6-difluorophenyl)-1-methyl-1H-pyrazol-4-yl]-7-(1-methyl-1H-pyrazol-3-yl)-6-nitroquinazoline

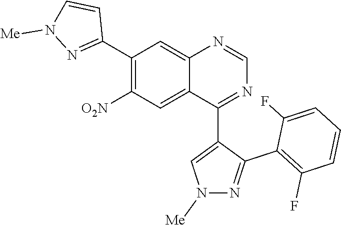 [SMILES: Cn1ccc(-c2cc3ncnc(-c4cn(C)nc4-c4c(F)cccc4F)c3cc2[N+](=O)[O-])n1]

The title compound was obtained as a yellow solid, 40 mg, 25% yield, from 4-bromo-7-(1-methyl-1H-pyrazol-3-yl)-6-nitroquinazoline (Preparation 283) and 3-(2,6-difluorophenyl)-1-methyl-4-(4,4,5,5-tetramethyl-1,3,2-dioxaborolan-2-yl)-1H-pyrazole (Preparation 34) following a similar procedure to that described in Preparation 274. LCMS m/z=448 [M+H]⁺.

### Preparation 285: 4-(3-(2,6-difluorophenyl)-1-methyl-1H-pyrazol-4-yl)-7-(1-methyl-1H-pyrazol-3-yl)quinazolin-6-amine

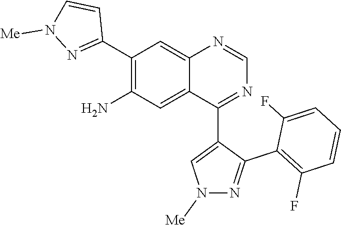 [SMILES: Cn1ccc(-c2cc3ncnc(-c4cn(C)nc4-c4c(F)cccc4F)c3cc2N)n1]

To a solution of 4-(3-(2,6-difluorophenyl)-1-methyl-1H-pyrazol-4-yl]-7-(1-methyl-1H-pyrazol-3-yl)-6-nitroquinazoline (Preparation 284, 40 mg, 0.089 mmol) was added iron (49.9 mg, 0.894 mmol) and NH₄Cl (47.8 mg, 0.894 mmol) in EtOH (3 mL) and H₂O (1 mL). The reaction mixture was stirred at 80° C. for 2 h, then filtered and concentrated under reduced pressure. The residue was purified by prep-TLC (10:1 DCM/MeOH) to afford the title compound as a yellow solid (30 mg, 80%).

### Preparation 286: methyl 3-amino-5,6-dichloropicolinate

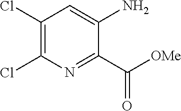 [SMILES: COC(=O)c1nc(Cl)c(Cl)cc1N]

NCS (749 mg, 5.59 mmol) was added to methyl 3-amino-5-chloropyridine-2-carboxylate (950 mg, 5.09 mmol) in MeCN (10 mL) at rt. The resulting mixture was heated to 80° C. for 16 h. The reaction mixture was diluted with EtOAc (100 mL), washed with water (100 mL×3) and saturated brine (100 mL). The organic layer was dried over Na₂SO₄, filtered and evaporated to afford crude product. This was purified by silica gel column eluting with PE:EtOAc=4:1 to give the title compound (1.1 g, yield: 98.2%) as a yellow solid. LCMS m/z=221 [M+H]⁺.

### Preparation 287: 6,7-dichloropyrido[3,2-d]pyrimidin-4 (3H)-one

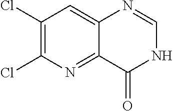 [SMILES: O=c1[nH]cnc2cc(Cl)c(Cl)nc12]

AcOH (1 mL) was added to methyl 3-amino-5,6-dichloropicolinate (Preparation 286, 1.1 g, 4.97 mmol) in formamide (10 mL) at rt and the reaction was heated to 120° C. for 16 h. The reaction mixture was diluted with EtOAc (100 mL), washed with water (100 mL×3) and saturated brine (100 mL). The organic layer was dried over Na₂SO₄, filtered and evaporated. The crude product was purified by silica gel column with DCM:MeOH=20:1 to give the title compound (900 mg, yield: 84.1%) as a brown solid. LCMS m/z=215 [M+H]⁺.

### Preparation 288: 7-chloro-6-((4-methoxybenzyl)amino)pyrido[3,2-d]pyrimidin-4 (3H)-one

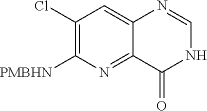

1-(4-Methoxyphenyl) methanamine (2.85 g, 20.8 mmol) was added to 6,7-dichloropyrido[3,2-d]pyrimidin-4 (3H)-one (Preparation 287, 900 mg, 4.16 mmol) in DMSO (10 mL) and the resulting mixture was heated at 80° C. for 16 h. The reaction mixture was diluted with EtOAc (100 mL), washed with water (100 mL×3) and saturated brine (100 mL). The organic layer was dried over Na₂SO₄, filtered and evaporated. The crude product was purified by prep-TLC with DCM:MeOH=20:1 to give the title compound (900 mg, yield: 68.7%) as a brown solid. LCMS m/z=317 [M+H]⁺.

### Preparation 289: 4,7-dichloro-N-(4-methoxybenzyl)pyrido[3,2-d]pyrimidin-6-amine

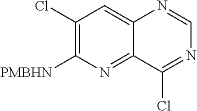

N,N-Diethylaniline (2.95 g, 19.8 mmol) was added to POCl₃ (2.17 g, 14.2 mmol) and 7-chloro-6-((4-methoxybenzyl)amino)pyrido[3,2-d]pyrimidin-4 (3H)-one (Preparation 288, 900 mg, 2.84 mmol) in MeCN (15 mL) at rt. The resulting mixture was heated at 80° C. for 2 h. The resulting solution was added to ice-water, then 1 M Na₂CO₃ was added until pH=8. The solution was extracted with EtOAc and concentrated under vacuum. The residue was purified by silica gel column with DCM:EtOAc=3:1 to give the title compound (360 mg, yield: 37.8%) as a yellow solid. LCMS m/z=335 [M+H]⁺.

### Preparation 290: 7-chloro-N-(4-methoxybenzyl)-4-(1-methyl-3-phenyl-1H-pyrazol-4-yl)pyrido[3,2-d]pyrimidin-6-amine

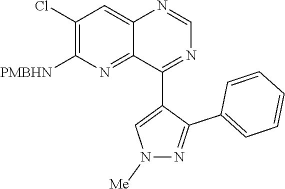

A mixture of Pd(dppf)Cl₂ (76.0 mg, 0.104 mmol), K₃PO₄ (330 mg, 1.56 mmol), 4,7-dichloro-N-(4-methoxybenzyl)pyrido[3,2-d]pyrimidin-6-amine (Preparation 289, 350 mg, 1.04 mmol) and 1-methyl-3-phenyl-4-(4,4,5,5-tetramethyl-1,3,2-dioxaborolan-2-yl)-1H-pyrazole (295 mg, 1.04 mmol) in dioxane/H₂O (8 mL/2 mL) was heated at 80° C. for 16 h under N₂. The reaction mixture was diluted with EtOAc (100 mL), washed with water (100 mL×3) and saturated brine (100 mL). The organic layer was dried over Na₂SO₄, filtered and evaporated. The crude product was purified by silica gel column with DCM:MeOH=20:1 to give the title compound (350 mg, yield: 73.6%) as a yellow solid. LCMS m/z=457 [M+H]⁺.

### Preparation 291: 7-chloro-4-(1-methyl-3-phenyl-1H-pyrazol-4-yl)pyrido[3,2-d]pyrimidin-6-amine

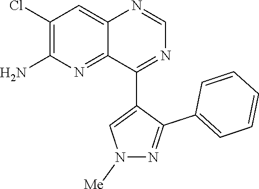 [SMILES: Cn1cc(-c2ncnc3cc(Cl)c(N)nc23)c(-c2ccccc2)n1]

A solution of 7-chloro-N-(4-methoxybenzyl)-4-(1-methyl-3-phenyl-1H-pyrazol-4-yl)pyrido[3,2-d]pyrimidin-6-amine (Preparation 290, 340 mg, 0.744 mmol) in TFA (3 mL) was heated to 60° C. for 2 h. The mixture was concentrated under vacuum. The crude product was purified by prep-TLC with DCM:MeOH=10:1 to give the title compound (160 mg, yield: 64.0%) as a yellow solid. LCMS m/z=337 [M+H]⁺.

### Preparation 292: N-(7-chloro-4-(1-methyl-3-phenyl-1H-pyrazol-4-yl)pyrido[3,2-d]pyrimidin-6-yl)-3-oxabicyclo[3.1.0]hexane-1-carboxamide

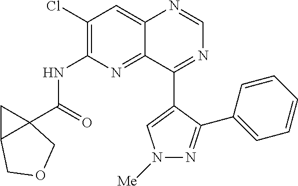 [SMILES: Cn1cc(-c2ncnc3cc(Cl)c(NC(=O)C45COCC4C5)nc23)c(-c2ccccc2)n1]

2,4,6-Trichlorobenzoyl chloride (324 mg, 1.33 mmol) was added to TEA (224 mg, 2.22 mmol) and 3-oxabicyclo[3.1.0]hexane-1-carboxylic acid (169 mg, 1.33 mmol) in THF (10 mL) at 0° C. After stirring for 1 h, 7-chloro-4-(1-methyl-3-phenyl-1H-pyrazol-4-yl)pyrido[3,2-d]pyrimidin-6-amine (Preparation 291, 150 mg, 0.445 mmol) was added and the mixture was stirred for 80° C. for 16 h. The reaction mixture was diluted with EtOAc (100 mL), washed with water (100 mL×3) and brine (100 mL). The organic layer was dried over Na₂SO₄, filtered and evaporated to afford crude product. The crude product was purified by prep-TLC with DCM:MeOH=20:1. The residue was purified by Prep-HPLC (Column: XBridge Prep OBD C18 Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 30% B to 50% B in 8 min, to give the title compound, 34.1 mg, 17.2% as a white solid. LCMS: m/z=447 [M+H]⁺, ¹H NMR (400 MHZ, DMSO-d₆) δ 10.10 (s, 1H), 9.03 (s, 1H), 8.94 (s, 1H), 8.64 (s, 1H), 7.52-7.45 (m, 2H), 7.31 (q, J=2.9 Hz, 3H), 4.06-3.98 (m, 5H), 3.80 (s, 2H), 2.36 (t, J=6.5 Hz, 1H), 1.56 (dd, J=8.3, 4.5 Hz, 1H), 0.99 (t, J=4.9 Hz, 1H).

### Preparation 293: 6-(4-methoxybenzyl)amino)-4-(1-methyl-3-phenyl-1H-pyrazol-4-yl)pyrido[3,2-d]pyrimidin-7-ol

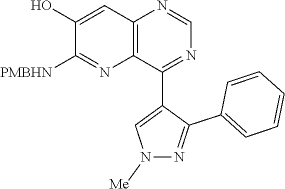

L-Selectride (1.67 g, 8.82 mmol) was added to 7-methoxy-N-(4-methoxybenzyl)-4-(1-methyl-3-phenyl-1H-pyrazol-4-yl)pyrido[3,2-d]pyrimidin-6-amine (Preparation 212, 2 g, 4.41 mmol) in THF (16 mL) and the reaction mixture was stirred at 80° C. for 2 h, then diluted with EtOAc (200 mL) and washed with brine (2×10 mL). The organic layer was dried over Na₂SO₄ and concentrated under reduced pressure. The residue was purified by column chromatography (SiO₂, 25:1 DCM/MeOH) to afford the title compound as a white solid (1.1 g, 55%). LCMS m/z=439 [M+H]⁺.

### Preparation 294: 6-((4-methoxybenzyl)amino)-4-(1-methyl-3-phenyl-1H-pyrazol-4-yl)pyrido[3,2-d]pyrimidin-7-yl trifluoromethanesulfonate

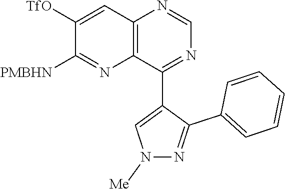

Tf₂O (321 mg, 1.14 mmol) was added to the solution of 6-((4-methoxybenzyl)amino)-4-(1-methyl-3-phenyl-1H-pyrazol-4-yl)pyrido[3,2-d]pyrimidin-7-ol (Preparation 293, 500 mg, 1.14 mmol), DMAP (137 mg, 0.57 mmol) and pyridine (456 mg, 5.70 mmol) in DCM (20 mL) at rt. The reaction mixture was stirred at rt for 2 h, then was diluted with EtOAc (100 mL) and washed with brine (2×50 mL). The organic layer was dried with Na₂SO₄ and concentrated under reduced pressure. The residue was purified by column chromatography (SiO₂, 15:1 DCM/MeOH) to afford the title compound as a yellow solid (160 mg, 32%). LCMS m/z=571 [M+H]⁺.

### Preparation 295: N-(4-methoxybenzyl)-4-(1-methyl-3-phenyl-1H-pyrazol-4-yl)-7-(3-morpholinoprop-1-yn-1-yl)pyrido[3,2-d]pyrimidin-6-amine

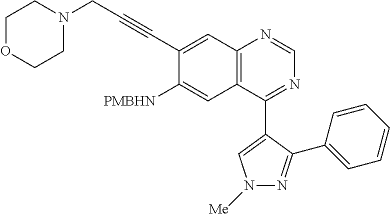

CuI (10.0 mg, 0.524 mmol) was added to a mixture of TEA (119 mg, 1.04 mmol), 6-((4-methoxy benzyl)amino)-4-(1-methyl-3-phenyl-1H-pyrazol-4-yl)pyrido[3,2-d]pyrimidin-7-yl trifluoromethane sulfonate (Preparation 294, 150 mg, 0.262 mmol) and 4-(prop-2-yn-1-yl)morpholine (98.3 mg, 0.786 mmol) in DME (5 mL) and the reaction was stirred at 80° C. for 2 h under N₂. The mixture was diluted with EtOAc (150 mL) and washed with brine (2×75 mL). The organic layer was dried over Na₂SO₄ and concentrated under reduced pressure. The residue was purified by column chromatography (SiO₂, 15:1 DCM/EtOAc) to afford the title compound as an off-white solid (80 mg, 54%). LCMS m/z=546 [M+H]⁺.

### Preparation 296: 4-(1-methyl-3-phenyl-1H-pyrazol-4-yl)-7-(3-morpholinoprop-1-yn-1-yl)pyrido[3,2-d]pyrimidin-6-amine

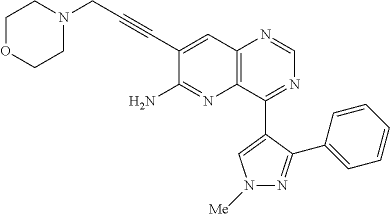 [SMILES: Cn1cc(-c2ncnc3cc(C#CCN4CCOCC4)c(N)nc23)c(-c2ccccc2)n1]

TFA (5 mL) was added to N-(4-methoxybenzyl)-4-(1-methyl-3-phenyl-1H-pyrazol-4-yl)-7-(3-morpholinoprop-1-yn-1-yl)pyrido[3,2-d]pyrimidin-6-amine (Preparation 295, 80 mg, 0.146 mmol) at rt. The reaction mixture was stirred at 60° C. for 3 h, then diluted with EtOAc (100 mL) and washed with brine (2×50 mL). The organic layer was dried with Na₂SO₄ and concentrated under reduced pressure. The residue was purified by column chromatography (SiO₂, 18:1 DCM/MeOH) to afford the title compound as a yellow solid (50 mg, 63%). LCMS m/z=426 [M+H]⁺.

### Preparation 297: 2-(4-(6-bromo-7-methoxyquinazolin-4-yl)-3-phenyl-1H-pyrazol-1-yl)-N,N-dimethylethan-1-amine

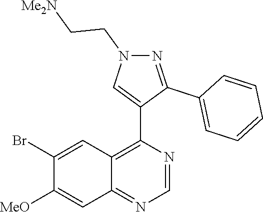 [SMILES: COc1cc2ncnc(-c3cn(CCN(C)C)nc3-c3ccccc3)c2cc1Br]

NaH (60%, 66.6 mg, 1.67 mmol) was added to a solution of 6-bromo-7-methoxy-4-(3-phenyl-1H-pyrazol-4-yl)quinazoline (Preparation 107, 320 mg, 839 μmol), (2-bromoethyl)dimethylamine hydrobromide (389 mg, 1.67 mmol) in DMF (15 mL) and the mixture stirred at 50° C. for 2 h. The reaction was quenched with water and evaporated to dryness in vacuo. The residue was purified by prep-TLC (20:1 DCM/MeOH) to afford the title compound as a yellow solid (100 mg, 10.5%). LCMS m/z=452,454 [M+H]⁺.

### Preparation 298: tert-butyl 3-carbamoyl-3-(trifluoromethyl) pyrrolidine-1-carboxylate

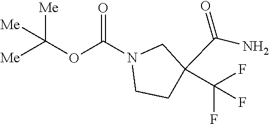 [SMILES: CC(C)(C)OC(=O)N1CCC(C(N)=O)(C(F)(F)F)C1]

To a solution of 1-(tert-butoxycarbonyl)-3-(trifluoromethyl) pyrrolidine-3-carboxylic acid (300 mg, 1.05 mmol) in DCM (10 mL) was added EDCl (256 mg, 1.65 mmol) and HOBt (222 mg, 1.65 mmol) and the mixture stirred at rt for 2 h before NH₃ (35% aq, 3 mL) was added. The resulting mixture was stirred at rt for 1 h and then concentrated to dryness. The residue was purified by prep-TLC (15:1 DCM/MeOH) to afford the title compound as a grey solid (200 mg, 66%) which was used without further purification.

### Preparation 299: 6-chloro-4-(1-ethyl-3-(4-fluorophenyl)-1H-pyrazol-4-yl)-7-methoxypyrido[3,2-d]pyrimidine

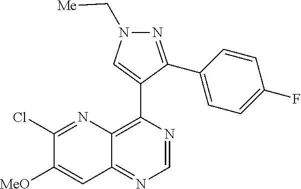 [SMILES: CCn1cc(-c2ncnc3cc(OC)c(Cl)nc23)c(-c2ccc(F)cc2)n1]

Into a 20-mL pressure tank reactor purged and maintained with an inert atmosphere of nitrogen, was placed 1-ethyl-3-(4-fluorophenyl)-4-(4,4,5,5-tetramethyl-1,3,2-dioxaborolan-2-yl)-1H-pyrazole (Preparation 44, 400 mg, 1.26 mmol), K₂CO₃ (324 mg, 2.52 mmol), Pd(dppf)Cl₂ (92 mg, 0.126 mmol), 4,6-dichloro-7-methoxypyrido[3,2-d]pyrimidine (Preparation 224, 289 mg, 1.26 mmol), dioxane (5 mL) and water (1 mL) and the resulting solution was stirred for 2 hr at 80° C. The reaction mixture was applied onto a silica gel column and eluted with 20:1 DCM/MeOH to afford the title compound as an off-white solid (200 mg, 41%). LCMS: m/z=384 [M+H]⁺.

### Preparation 300: (1S,5S)-3-oxabicyclo[3.1.0]hexane-1-carboxamide

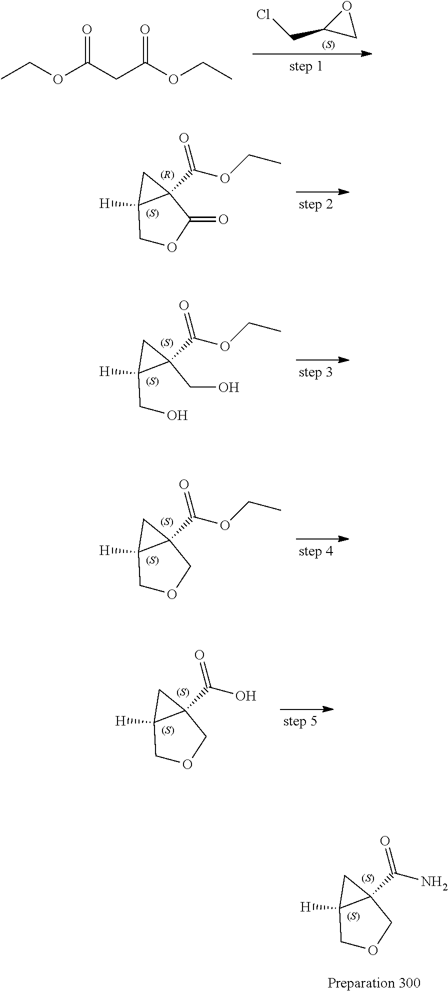

### Step 1: ethyl (1R,5S)-2-oxo-3-oxabicyclo[3.1.0]hexane-1-carboxylate

To a solution of diethyl malonate (228 g, 1.42 mol) in ethanol (1.2 L) was added 20% sodium ethoxide (463 g, 1.36 mol) ethanol solution at 20° C. slowly. The mixture was stirred at 20° C. for 0.5 hour under nitrogen. Then, to the mixture was added a solution of(S)-2-(chloromethyl) oxirane (120 g, 1.30 mol) in ethanol (200 mL) at 20° C. The mixture was stirred at 80° C. for 16 hours under nitrogen. On completion, after cooled to 25° C., the mixture was concentrated in vacuo. The residue was dissolved in ethyl acetate (1.0 L) and aqueous hydrochloric acid (1.0 M, 1.4 L) and partitioned. The aqueous phase was extracted with ethyl acetate (700 mL×2). The organic layer was washed with brine (300 mL), dried over anhydrous sodium sulfate and concentrated in vacuo. The residue was purified by column chromatography on silica gel (petroleum ether:ethyl acetate=10:1 to 3:1) to give the title compound (168 g, 76% yield) as a light yellow oil.

### Step 2: ethyl (1S,2S)-1,2-bis(hydroxymethyl)cyclopropane-1-carboxylate

The reaction was performed for two batches in parallel: To a solution of ethyl (1R,5S)-2-oxo-3-oxabicyclo[3.1.0]hexane-1-carboxylate (84.0 g, 493 mmol) in ethanol (850 mL) was added sodium borohydride (14.9 g, 395 mmol) in portions at 0˜5° C. The mixture was stirred at 20° C. for 2 hours. On completion, the reaction was quenched with aqueous hydrochloric acid (0.2 M, 100 mL) at 0° C., the mixture was stirred at 20° C. for 0.5 hour. The mixture of two batches was combined and concentrated in vacuo. The residue was diluted with brine (200 mL), extracted with dichloromethane/isopropanol=3:1 (100 mL×3). The organic layer was dried over sodium sulfate, filtered and concentrated in vacuo to give the title compound (136 g, 79% yield) as a light yellow oil.

### Step 3: ethyl (1S,5S)-3-oxabicyclo[3.1.0]hexane-1-carboxylate

The reaction was performed for two batches in parallel: To a solution of ethyl (1S,2S)-1,2-bis(hydroxymethyl)cyclopropane-1-carboxylate (63.0 g. 361 mmol), triethylamine (110 g, 1.09 mol) and N,N-dimethylpyridin-4-amine (8.84 g. 72.3 mmol) in dichloromethane (1.2 L) was added p-toluenesulfonylchloride (138 g, 723 mmol) slowly at 0-5° C. The mixture was stirred at 20° C. for 16 hours under nitrogen. On completion, the mixture of two batches were combined. The mixture was washed with aqueous hydrochloric acid (2.0 M, 1.2 L). The aqueous phase was extracted with dichloromethane (1.0 L). The combined organic layer was washed with brine (500 mL), dried over sodium sulfate, filtered and concentrated in vacuo. The residue was purified by column chromatography on silica gel (petroleum ether:ethyl acetate=20:1) to give the title compound (130 g, crude) as a light yellow oil.

### Step 4: (1S,5S)-3-oxabicyclo[3.1.0]hexane-1-carboxylic acid

To a solution of ethyl (1S,5S)-3-oxabicyclo[3.1.0]hexane-1-carboxylate (130 g, crude) in tetrahydrofuran (1.2 L) and methanol (200 mL) was added a solution of lithium hydroxide hydrate (70.0 g. 1.67 mol) in water (400 mL) at 20° C. The mixture was stirred at 20° C. for 3 hours. On completion, the mixture was diluted with water (2.5 L), extracted with tert-butylmethylether (1.0 L×3). The aqueous phase was acidified with potassium hydrogen sulfate solid (700 g. 5.14 mol), the mixture was stirred for 0.5 hour. To the mixture was added ethyl acetate (1.5 L), the mixture was filtered and partitioned. The aqueous phase was extracted with ethyl acetate (1.5 L×2). The combined organic layer was washed with brine (500 mL), dried over sodium sulfate, filtered and concentrated in vacuo to give the title compound as a yellow oil. ¹H-NMR line list data: ¹H NMR (400 MHZ, 6d-DMSO): δ ppm 12.98-11.69 (m, 1H), 3.85 (d, J=8.4 Hz, 1H), 3.75 (d, J=8.4 Hz, 1H), 3.72-3.68 (m, 1H), 3.68-3.64 (m, 1H), 2.08-2.04 (m, 1H), 1.33 (dd, J=8.0, 4.0 Hz, 1H), 0.85-0.82 (m, 1H).

### Step 5: (1S,5S)-3-oxabicyclo[3.1.0]hexane-1-carboxamide

To a mixture of (1S,5S)-3-oxabicyclo[3.1.0]hexane-1-carboxylic acid (1S,5S)-3-oxabicyclo[3.1.0]hexane-1-carboxylic acid (100 mg, 781 μmol) in DCM (10 mL) was added EDCl (225 mg, 1.17 mmol), HOBt (158 mg, 1.17 mmol). Stirred at r.t. for 1 h. Then added NH₃ (35% aq, 2 mL) was added and stirred at r.t. for 30 min. Concentrated to dryness. The residue was purified on prep-TLC with DCM:MeOH=15:1 to afford title compound (75 mg, yield: 75%) as a white solid. LC-MS: (ES, m/z): RT=0.913 min, LCMS: m/z=128 [M+1].

### Preparation 301: 7-methoxy-4-(1-methyl-3-phenyl-1H-pyrazol-4-yl)-6-(4,4,5,5-tetramethyl-1,3,2-dioxaborolan-2-yl)quinazoline

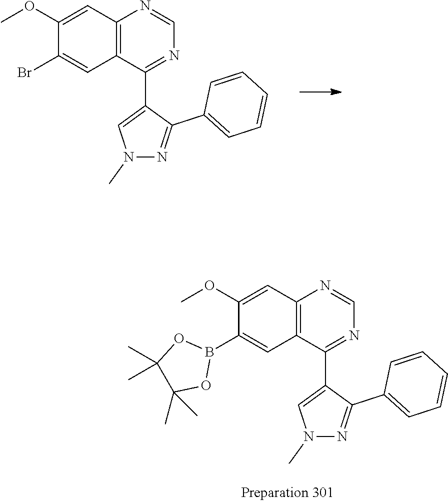 [SMILES: COc1cc2ncnc(-c3cn(C)nc3-c3ccccc3)c2cc1Br ; COc1cc2ncnc(-c3cn(C)nc3-c3ccccc3)c2cc1B1OC(C)(C)C(C)(C)O1]

To a solution of 6-bromo-7-methoxy-4-(1-methyl-3-phenyl-1H-pyrazol-4-yl)quinazoline (Preparation 91, 100 mg, 253 μmol), 2-isopropoxy-4,4,5,5-tetramethyl-1,3,2-dioxaborolane (271 mg, 506 μmol) in THF (10 mL) was added BuLi (0.2 mL, 2.5 M in hexane, 506 μmol) at −78° C. Stirred at −10° C. for 1 h. Quenched with water. Extracted with EtOAC. The organic layer was dried over Na₂SO₄. Filtered and concentrated to dryness to afford title compound (150 mg, crude) as a grey solid. LC-MS: (ES, m/z): RT=1.235 min, LCMS: m/z=443 [M+1].

### Preparation 302: tert-butyl (1S,5S)-1-carbamoyl-3-azabicyclo[3.1.0]hexane-3-carboxylate

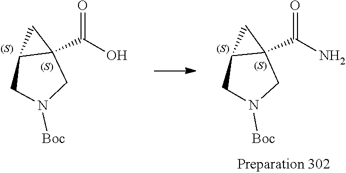

To a mixture of (1S,5S)-3-(tert-butoxycarbonyl)-3-azabicyclo[3.1.0]hexane-1-carboxylic acid (100 mg, 440 μmol) in DCM (10 mL) was added EDCl (126 mg, 660 μmol), HOBt (89 mg, 660 μmol). Stirred at r.t. for 1 h. Then added NH₃ (35% aq, 2 mL) was added and stirred at r.t. for 30 min. Concentrated to dryness. The residue was purified on prep-TLC with DCM:MeOH=15:1 to afford title compound (70 mg, yield: 70%) as a white solid. LC-MS: (ES, m/z): RT=0.871 min, LCMS: m/z=171 [M-55]

### Preparation 303: 7-methoxy-6-nitro-4-(3-phenyl-1H-pyrazol-4-yl)quinazoline

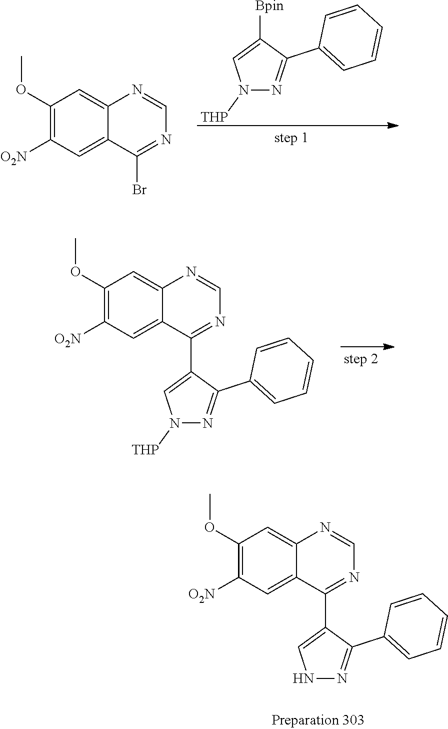

### Step 1: Synthesis of 7-methoxy-6-nitro-4-[1-(oxan-2-yl)-3-phenyl-1H-pyrazol-4-yl]quinazoline

The reaction mixture of 4-bromo-7-methoxy-6-nitroquinazoline (568 mg, 2 mmol), 1-(oxan-2-yl)-3-phenyl-4-(4,4,5,5-tetramethyl-1,3,2-dioxaborolan-2-yl)-1H-pyrazole (1.05 g, 3 mmol), Pd(dppf)Cl₂ (155 mg, 0.2 mmol) and K₂CO₃ (549 mg, 4 mmol) in dioxane (8 mL) and H₂O (2 mL) was stirred at 80° C. for 4 hrs, then poured into water, and the title compound (900 mg, crude). LC-MS: (ES, m/z): RT=1.319 min, LCMS: m/z=432 [M+1].

### Step 2: Synthesis of 7-methoxy-6-nitro-4-(3-phenyl-1H-pyrazol-4-yl)quinazoline

7-methoxy-6-nitro-4-[1-(oxan-2-yl)-3-phenyl-1H-pyrazol-4-yl]quinazoline (500 mg, 1.15 mmol) was added to HCl in Dioxane (5 mL) and stirred for 2 hrs, then the title compound was collected by filter (400 mg, yield: 90%). LC-MS: (ES, m/z): RT=1.107 min, LCMS: m/z=347.9 [M+1].

### PREPARATION OF EXAMPLES

### Example 1: 6-(((3R,4R)-3-fluoro-1-methylpiperidin-4-yl)oxy)-7-methoxy-4-(1-methyl-3-phenyl-1H-pyrazol-4-yl)quinazoline

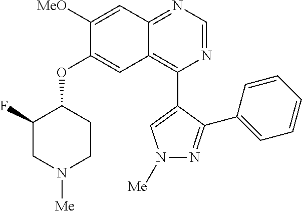 [SMILES: COc1cc2ncnc(-c3cn(C)nc3-c3ccccc3)c2cc1O[C@@H]1CCN(C)C[C@H]1F]

Part 1: TFA (5 mL) was added to tert-butyl (3R,4R)-3-fluoro-4-((7-methoxy-4-(1-methyl-3-phenyl-1H-pyrazol-4-yl)quinazolin-6-yl)oxy) piperidine-1-carboxylate (Preparation 119, 250 mg, 0.468 mmol) in DCM (15 mL) and the reaction was stirred at rt for 1 h. The mixture was concentrated under vacuum to afford 6-(((3R,4R)-3-fluoropiperidin-4-yl)oxy)-7-methoxy-4-(1-methyl-3-phenyl-1H-pyrazol-4-yl)quinazoline trifluoroacetate, 150 mg as a yellow oil.

Part 2: This oil was dissolved in DCM, formaldehyde (0.5 mL) and Na(OAc)₃BH (73.3 mg, 0.346 mmol) added and the reaction stirred at rt for 16 h. The mixture was diluted with EtOAc (100 mL), washed with brine (50 mL×2), the organic layer was dried over Na₂SO₄ and concentrated under vacuum. The residue was purified by Prep HPLC: Column: XBridge Prep OBD C18 Column, 30×150 mm 5 um; Mobile Phase A: Water (10 mmol/L NH₄HCO₃), Mobile Phase B: MeCN; Flow rate: 60 mL/min; Gradient: 20% B to 40% B in 8 min; to afford the title compound as a white solid, 20.5 mg. LCMS: m/z=448 [M+H]⁺, ¹H NMR (DMSO-d₆, 400 MHz): ppm δ=9.08 (s, 1H), 8.22 (s, 1H), 7.37 (s, 1H), 7.32-7.19 (m, 5H), 6.99 (s, 1H), 4.52 (dtd, 1H), 4.03 (s, 3H), 3.96 (s, 3H), 3.86-3.73 (m, 1H), 2.95 (td, 1H), 2.55 (s, 1H), 2.24 (s, 3H), 2.12 (td, 1H), 2.01-1.91 (m, 1H), 1.50 (d, 1H), 1.36-1.22 (m, 1H).

### Example 2: 6-(((3S,4S)-3-fluoro-1-methylpiperidin-4-yl)oxy)-7-methoxy-4-(1-methyl-3-phenyl-1H-pyrazol-4-yl)quinazoline

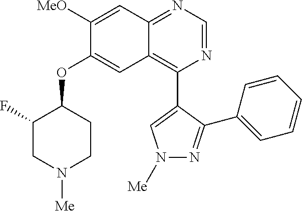 [SMILES: COc1cc2ncnc(-c3cn(C)nc3-c3ccccc3)c2cc1O[C@H]1CCN(C)C[C@@H]1F]

The title compound was obtained as a white solid, 35.7 mg, 17%, from tert-butyl (3S,4S)-3-fluoro-4-((7-methoxy-4-(1-methyl-3-phenyl-1H-pyrazol-4-yl)quinazolin-6-yl)oxy) piperidine-1-carboxylate (Preparation 120) and formaldehyde, following the procedure described in Example 1. LCMS: m/z=448 [M+H]⁺, ¹H NMR (400 MHZ, DMSO-d₆) ppm δ=9.08 (s, 1H), 8.22 (s, 1H), 7.37 (s, 1H), 7.32-7.19 (m, 5H), 6.99 (s, 1H), 4.52 (d, 1H, J=49.8 Hz), 4.02 (s, 3H), 3.96 (s, 3H), 3.80 (s, 1H), 2.96 (s, 1H), 2.25 (s, 3H), 2.13 (s, 1H), 2.20-1.97 (m, 1H), 1.49 (bs, 1H), 1.29-1.28 (m, 1H), 1.23-1.22 (m, 1H).

### Example 3:4-(1-(2,2-difluoroethyl)-3-phenyl-1H-pyrazol-4-yl)-6-(((3R,4R)-3-fluoro-1-methylpiperidin-4-yl)oxy)-7-methoxyquinazoline

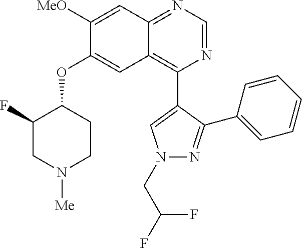 [SMILES: COc1cc2ncnc(-c3cn(CC(F)F)nc3-c3ccccc3)c2cc1O[C@@H]1CCN(C)C[C@H]1F]

The title compound was obtained as a white solid, 81.7 mg, 66% yield, from tert-butyl (3R,4R)-4-((4-(1-(2,2-difluoroethyl)-3-phenyl-1H-pyrazol-4-yl)-7-methoxyquinazolin-6-yl)oxy)-3-fluoropiperidine-1-carboxylate (Preparation 123) and formaldehyde, following a similar procedure to that described in Example 1, except the crude product was purified by Column: YMC-Actus Triart C18 ExRS, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 23% B to 56% B in 7 min, 56% B; LCMS: m/z=498 [M+H]⁺, ¹H NMR (400 MHZ, DMSO-d₆) δ 9.08 (s, 1H), 8.34 (s, 1H), 7.40 (s, 1H), 7.31-7.26 (m, 5H), 7.03 (s, 1H), 6.55 (t, 1H), 4.83 (td, 2H), 4.66-4.56 (m, 1H), 3.98 (s, 3H), 3.88 (s, 1H), 2.97 (s, 1H), 2.57 (s, 1H), 2.24 (s, 3H), 2.11 (td, 1H), 2.01-1.91 (m, 1H), 1.60 (d, 1H), 1.41-1.27 (m, 1H).

### Example 4 and 5: 6-(((3S,4R)-4-fluoro-1-methylpyrrolidin-3-yl)oxy)-7-methoxy-4-(1-methyl-3-phenyl-1H-pyrazol-4-yl)quinazoline and 6-(((3R,4S)-4-fluoro-1-methylpyrrolidin-3-yl)oxy)-7-methoxy-4-(1-methyl-3-phenyl-1H-pyrazol-4-yl)quinazoline

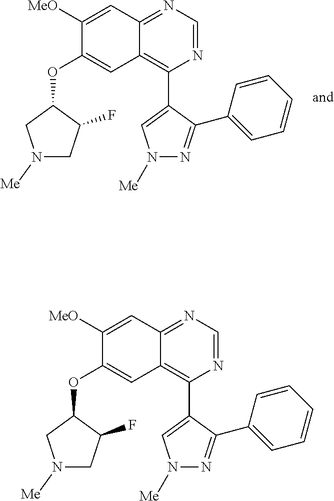 [SMILES: COc1cc2ncnc(-c3cn(C)nc3-c3ccccc3)c2cc1O[C@H]1CN(C)C[C@H]1F ; COc1cc2ncnc(-c3cn(C)nc3-c3ccccc3)c2cc1O[C@@H]1CN(C)C[C@@H]1F]

Cis-rac-6-(((3S,4R)-4-fluoro-1-methylpyrrolidin-3-yl)oxy)-7-methoxy-4-(1-methyl-3-phenyl-1H-pyrazol-4-yl)quinazoline was obtained as a white solid, 170 mg, 65% yield from cis-rac-tert-butyl (3S,4R)-3-fluoro-4-((7-methoxy-4-(1-methyl-3-phenyl-1H-pyrazol-4-yl)quinazolin-6-yl)oxy) piperidine-1-carboxylate (Preparation 121) and formaldehyde following a similar procedure to that described in Example 1, except the crude was purified by YMC-Actus Triart C18 ExRS, 30*150 mm. 5 μm; Mobile Phase A: Water (10 mmol/L NH₄HCO₃+0.1% NH₃·H₂O), Mobile Phase B: MeCN: Flow rate: 60 mL/min; Gradient: 19% B to 49% B in 7 min.

This compound was further purified by chiral prep-HPLC Column: CHIRALPAK IF. 2*25 cm, 5 μm; Mobile Phase A: Hex:DCM=3:1 (0.5% 2M NH₃—MeOH), Mobile Phase B: IPA: Flow rate: 20 mL/min; Gradient: 10% B isocratic; to give Peak 1, (86.7 mg, yield: 51%) as a white solid, Example 4, (6-(((3S,4R)-4-fluoro-1-methylpyrrolidin-3-yl)oxy)-7-methoxy-4-(1-methyl-3-phenyl-1H-pyrazol-4-yl)quinazoline or 6-(((3R,4S)-4-fluoro-1-methylpyrrolidin-3-yl)oxy)-7-methoxy-4-(1-methyl-3-phenyl-1H-pyrazol-4-yl)quinazoline. LCMS: m/z=434 [M+H]⁺, ¹H NMR (400 MHZ, DMSO-d₆) δ 9.07 (s, 1H), 8.24 (s, 1H), 7.38 (s, 1H), 7.34-7.28 (m, 2H), 7.27-7.22 (m, 3H), 6.99 (s, 1H), 4.96-4.77 (m, 1H), 4.36 (dq, 1H), 4.03 (s, 3H), 3.97 (s, 3H), 2.89-2.75 (m, 2H), 2.69-2.56 (m, 2H), 2.30 (s, 3H).

Further elution provided Peak 2, 11.1 mg, 6% yield as a white solid, Example 5, (6-(((3R,4S)-4-fluoro-1-methylpyrrolidin-3-yl)oxy)-7-methoxy-4-(1-methyl-3-phenyl-1H-pyrazol-4-yl)quinazoline or 6-(((3S,4R)-4-fluoro-1-methylpyrrolidin-3-yl)oxy)-7-methoxy-4-(1-methyl-3-phenyl-1H-pyrazol-4-yl)quinazoline. LCMS: m/z=434 [M+H]⁺, ¹H NMR (400 MHZ, DMSO-d₆) δ 9.07 (s, 1H), 8.23 (s, 1H), 7.38 (s, 1H), 7.32 (dd, 2H), 7.26-7.22 (m, 3H), 6.98 (s, 1H), 4.85 (dtd, 1H), 4.31 (dq, 1H), 4.03 (s, 3H), 3.97 (s, 3H), 2.81-2.69 (m, 2H), 2.62-2.53 (m, 2H), 2.25 (s, 3H).

### Example 6 and 7: 6-(((3S,4S)-4-fluoro-1-methylpyrrolidin-3-yl)oxy)-7-methoxy-4-(1-methyl-3-phenyl-1H-pyrazol-4-yl)quinazoline and 6-(((3R,4R)-4-fluoro-1-methylpyrrolidin-3-yl)oxy)-7-methoxy-4-(1-methyl-3-phenyl-1H-pyrazol-4-yl)quinazoline

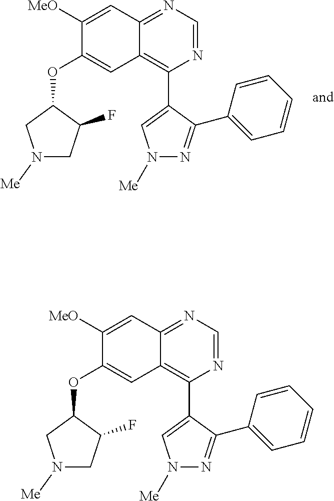 [SMILES: COc1cc2ncnc(-c3cn(C)nc3-c3ccccc3)c2cc1O[C@H]1CN(C)C[C@@H]1F ; COc1cc2ncnc(-c3cn(C)nc3-c3ccccc3)c2cc1O[C@@H]1CN(C)C[C@H]1F]

Trans-rac-6-(((3S,4S)-4-fluoro-1-methylpyrrolidin-3-yl)oxy)-7-methoxy-4-(1-methyl-3-phenyl-1H-pyrazol-4-yl)quinazoline was obtained as an off-white solid, 157 mg, 47%, from trans-rac-tert-butyl (3S,4S)-3-fluoro-4-((7-methoxy-4-(1-methyl-3-phenyl-1H-pyrazol-4-yl)quinazolin-6-yl)oxy) pyrrolidine-1-carboxylate (Preparation 122) and formaldehyde, following the procedure described in Example 4.

This was further purified by Prep-Chiral-HPLC: Column: Lux 5 μm Cellulose-4, 2.12*25 cm, 5 μm; Mobile Phase A: Hex (0.5% 2M NH₃—MeOH), Mobile Phase B: IPA; Flow rate: 20 mL/min; Gradient: 30% B isocratic, to give

Peak 1, Example 6, 51.6 mg, as a white solid, 6-(((3S,4S)-4-fluoro-1-methylpyrrolidin-3-yl)oxy)-7-methoxy-4-(1-methyl-3-phenyl-1H-pyrazol-4-yl)quinazoline and 6-(((3R,4R)-4-fluoro-1-methylpyrrolidin-3-yl)oxy)-7-methoxy-4-(1-methyl-3-phenyl-1H-pyrazol-4-yl)quinazoline. LCMS m/z=434 [M+H]⁺. ¹H NMR (400 MHZ, DMSO-d₆) δ 9.07 (s, 1H), 8.25 (s, 1H), 7.40 (s, 1H), 7.34-7.26 (m, 2H), 7.26-7.17 (m, 3H), 7.06 (s, 1H), 5.02 (d, 1H), 4.43 (dt, 1H), 3.99 (d, 6H), 2.92 (dd, 1H), 2.76 (t, 2H), 2.24 (s, 4H).

and Peak 2, 58.5 mg, as a white solid, Example 7, 6-(((3R,4R)-4-fluoro-1-methylpyrrolidin-3-yl)oxy)-7-methoxy-4-(1-methyl-3-phenyl-1H-pyrazol-4-yl)quinazoline or 6-(((3S,4S)-4-fluoro-1-methylpyrrolidin-3-yl)oxy)-7-methoxy-4-(1-methyl-3-phenyl-1H-pyrazol-4-yl)quinazoline. LCMS: m/z=434 [M+H]⁺, ¹H NMR (400 MHZ, DMSO-d₆) δ 9.07 (s, 1H), 8.25 (s, 1H), 7.40 (s, 1H), 7.34-7.26 (m, 2H), 7.26-7.17 (m, 3H), 7.06 (s, 1H), 5.02 (s, 1H), 4.43 (dt, 1H), 3.99 (d, 6H), 2.92 (dd, 1H), 2.76 (t, 2H), 2.24 (s, 4H).

### Example 8:7-ethoxy-4-(1-methyl-3-phenyl-1H-pyrazol-4-yl)-6-(piperidin-4-yloxy)quinazoline

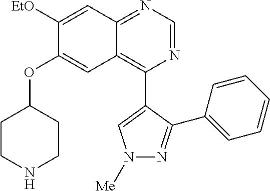 [SMILES: CCOc1cc2ncnc(-c3cn(C)nc3-c3ccccc3)c2cc1OC1CCNCC1]

TFA (3 mL) was added dropwise to tert-butyl 4-((7-ethoxy-4-(1-methyl-3-phenyl-1H-pyrazol-4-yl)quinazolin-6-yl)oxy) piperidine-1-carboxylate (Preparation 124, 70 mg, 0.132 mmol) in DCM (9 mL) and the reaction stirred at rt for 1 h. The mixture was concentrated under vacuum to afford crude product as a yellow oil. This was purified with prep-HPLC Column: XBridge Prep OBD C18 Column, 30×150 mm 5 um; Mobile Phase A: Water (10 mmol/L NH₄HCO₃+0.1% NH₃·H₂O), Mobile Phase B: MeCN: Flow rate: 60 mL/min; Gradient: 13% B to 43% B in 8 min, to give the title compound, as a white solid. LCMS m/z=430 [M+H]⁺ 1H NMR (400 MHZ, DMSO-d₆): ppm δ=9.06 (s, 1H), 8.20 (s, 1H), 7.32 (s, 1H), 7.30-7.24 (m, 2H), 7.24-7.19 (m, 3H), 6.89 (s, 1H), 4.22 (q, 2H), 4.02 (s, 3H), 3.82 (s, 1H), 2.84 (d, 2H), 2.40 (t, 2H), 1.50 (d, 2H), 1.40 (t, 3H), 1.22 (d, 2H).

### Example 9: 7-ethoxy-4-(1-methyl-3-phenyl-1H-pyrazol-4-yl)-6-((1-methylpiperidin-4-yl)oxy)quinazoline

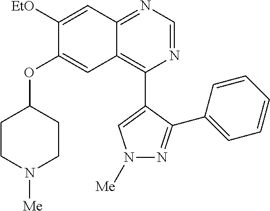 [SMILES: CCOc1cc2ncnc(-c3cn(C)nc3-c3ccccc3)c2cc1OC1CCN(C)CC1]

Na(OAc)₃BH (49.1 mg, 0.232 mmol) was added batchwise to 7-ethoxy-4-(1-methyl-3-phenyl-1H-pyrazol-4-yl)-6-((1-methylpiperidin-4-yl)oxy)quinazoline (Example 8, 50 mg, 0.116 mmol) and formaldehyde (25.5 mg, 0.580 mmol) in DCM (10 mL) and the reaction was stirred at rt for 2 h. The mixture was concentrated under vacuum and the residue was purified by Prep-HPLC Column: YMC-Actus Triart C18, 30 mm×150 mm, 5 μm; Mobile Phase A: Water (10 mmol/L NH₄HCO₃+0.1% NH₃·H₂O), Mobile Phase B: MeCN; Flow rate: 60 mL/min; Gradient: 40% B to 60% B in 8 min to give the title compound as a white solid, 12.8 mg, 25%. LCMS: m/z=444 [M+H]⁺, ¹H NMR (400 MHZ, DMSO-d₆) δ=9.06 (s, 1H), 8.20 (s, 1H), 7.33 (s, 1H), 7.31-7.21 (m, 2H), 7.25-7.19 (m, 3H), 6.89 (s, 1H), 4.22 (q, 2H), 4.02 (s, 3H), 3.83-3.76 (m, 1H), 2.55 (t, 2H), 2.16 (s, 3H), 2.00 (t, 2H), 1.51 (s, 2H), 1.44-1.31 (m, 5H).

### Example 10: 1-(4-((7-ethoxy-4-(1-methyl-3-phenyl-1H-pyrazol-4-yl)quinazolin-6-yl)oxy) piperidin-1-yl) propan-1-one

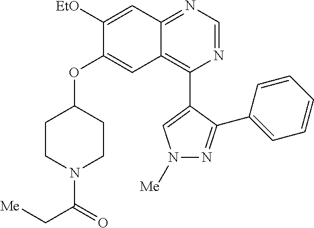 [SMILES: CCOc1cc2ncnc(-c3cn(C)nc3-c3ccccc3)c2cc1OC1CCN(C(=O)CC)CC1]

TEA (21.1 mg, 0.209 mmol) was added to an ice-cooled mixture of 7-ethoxy-4-(1-methyl-3-phenyl-1H-pyrazol-4-yl)-6-(piperidin-4-yloxy)quinazoline (Example 8, 30 mg, 0.07 mmol) and prop-2-enoyl chloride (12.8 mg, 0.139 mmol) in DCM (3 mL) and the reaction mixture was stirred at 25° C. for 1 h. The mixture was concentrated in vacuum and the residue was purified by prep-HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 22% B to 45% B in 10 min, to give the title compound (17.7 mg, yield: 52.3%) as a white solid. LCMS: m/z=486 [M+H]⁺. ¹H NMR (400 MHZ, DMSO-d₆) δ 9.07 (s, 1H), 7.35 (s, 1H), 7.32-7.26 (m, 2H), 7.26-7.21 (m, 3H), 6.97 (s, 1H), 4.23 (q, 2H), 4.12-4.06 (m, 1H), 4.02 (s, 3H), 3.78-3.69 (m, 1H), 3.57 (d, 1H), 3.19 (t, 1H), 3.10 (t, 1H), 2.32 (q, 2H), 1.59 (d, 1H), 1.48 (s, 1H), 1.39 (t, 4H), 1.22 (d, 1H), 0.99 (t, 3H).

### Example 11: 7-methoxy-4-(1-methyl-3-phenyl-1H-pyrazol-4-yl)-6-((tetrahydrofuran-3-yl)oxy)quinazoline

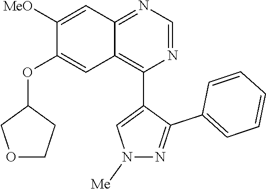 [SMILES: COc1cc2ncnc(-c3cn(C)nc3-c3ccccc3)c2cc1OC1CCOC1]

A mixture of 4-chloro-7-methoxy-6-((tetrahydrofuran-3-yl)oxy)quinazoline (Preparation 125, 80 mg, 0.284 mmol), 1-methyl-3-phenyl-4-(4,4,5,5-tetramethyl-1,3,2-dioxaborolan-2-yl)-1H-pyrazole (80 mg, 0.281 mmol), K₃PO₄ (59.6 mg, 0.281 mmol) and Pd(PPh₃)₄ (25 mg, 0.0216 mmol) in dioxane (15 mL) and water (4 mL) was stirred at 100° C. for 16 h. The reaction mixture was concentrated to dryness. The residue was purified on prep-TLC eluting with DCM:MeOH=20:1. The crude product was purified by prep-HPLC, Column: YMC-Actus Triart C18, 30*150 mm, 5 μm; Mobile Phase A: Water (10 mmol/L NH₂HCO₃+0.1% NH₃·H₂O), Mobile Phase B: MeCN; Flow rate: 60 mL/min; Gradient: 30% B to 45% B in 8 min, to afford the title compound, 38.9 mg as a white solid. LCMS: m/z=403 [M+H]⁺; ¹H NMR (300 MHz, DMSO-d₆) δ: 9.07 (s, 1H), 8.23 (s, 1H), 7.37 (s, 1H), 7.30 (dd, 2H), 7.27-7.18 (m, 3H), 6.86 (s, 1H), 4.47 (s, 1H), 4.03 (s, 3H), 3.96 (s, 3H), 3.76-3.51 (m, 4H), 1.94 (m, 1H), 1.73-1.63 (m, 1H).

### Example 12 and 13: (R)-7-methoxy-4-(1-methyl-3-phenyl-1H-pyrazol-4-yl)-6-((tetrahydrofuran-3-yl)oxy)quinazoline and (S)-7-methoxy-4-(1-methyl-3-phenyl-1H-pyrazol-4-yl)-6-((tetrahydrofuran-3-yl)oxy)quinazoline

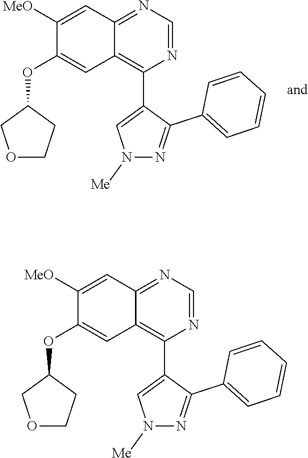 [SMILES: COc1cc2ncnc(-c3cn(C)nc3-c3ccccc3)c2cc1O[C@@H]1CCOC1 ; COc1cc2ncnc(-c3cn(C)nc3-c3ccccc3)c2cc1O[C@H]1CCOC1]

7-Methoxy-4-(1-methyl-3-phenyl-1H-pyrazol-4-yl)-6-((tetrahydrofuran-3-yl)oxy)quinazoline (Example 11, 30 mg) was further purified by Column: CHIRALPAK AD-H, 2*25 cm, 5 um; Mobile Phase A: Hex (0.5% 2M NH₃-MeOH), Mobile Phase B: EtOH; Flow rate: 40 mL/min; Gradient: 30% B isocratic to afford

Peak 1, 9.3 mg as a white solid, Example 12, (R)-7-methoxy-4-(1-methyl-3-phenyl-1H-pyrazol-4-yl)-6-((tetrahydrofuran-3-yl)oxy)quinazoline or (S)-7-methoxy-4-(1-methyl-3-phenyl-1H-pyrazol-4-yl)-6-((tetrahydrofuran-3-yl)oxy)quinazoline. LCMS: m/z=403 [M+H]⁺; ¹H NMR (400 MHz, DMSO-d₆) δ: 9.07 (s, 1H), 8.24 (s, 1H), 7.37 (s, 1H), 7.20-7.33 (m, 5H), 6.86 (s, 1H), 4.44-4.51 (m, 1H), 4.03 (s, 3H), 3.96 (s, 3H), 3.57-3.77 (m, 3H), 3.53 (d, 1H), 1.95 (m, 1H), 1.68 (m, 1H).

And Peak 2, 6.1 mg as a white solid, Example 13, (S)-7-methoxy-4-(1-methyl-3-phenyl-1H-pyrazol-4-yl)-6-((tetrahydrofuran-3-yl)oxy)quinazoline or (R)-7-methoxy-4-(1-methyl-3-phenyl-1H-pyrazol-4-yl)-6-((tetrahydrofuran-3-yl)oxy)quinazoline. LCMS: m/z=403 [M+H]⁺ 1H NMR (400 MHz, DMSO-d₆) δ=9.07 (s, 1H), 8.24 (s, 1H), 7.39-7.09 (m, 6H), 6.86 (s, 1H), 4.51-4.44 (m, 1H), 4.03 (s, 3H), 3.96 (s, 3H), 3.77-3.57 (m, 3H), 3.53 (d, 1H), 2.02-1.89 (m, 1H), 1.68 (m, 1H).

### Example 14:7-methoxy-4-(1-methyl-3-phenyl-1H-pyrazol-4-yl)-6-((tetrahydro-2H-pyran-4-yl)oxy)quinazoline

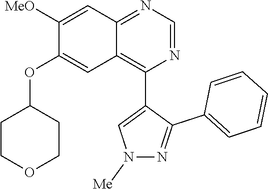 [SMILES: COc1cc2ncnc(-c3cn(C)nc3-c3ccccc3)c2cc1OC1CCOCC1]

The title compound was obtained as a white solid, 54 mg, 51% yield, from 4-chloro-7-methoxy-6-((tetrahydro-2H-pyran-4-yl)oxy)quinazoline (Preparation 126) and 1-methyl-3-phenyl-4-(4,4,5,5-tetramethyl-1,3,2-dioxaborolan-2-yl)-1H-pyrazole, following a similar procedure to that described in Example 11, except the compound was purified by prep-HPLC, Column: YMC-Actus Triart C18 ExRS, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 17% B to 50% B in 7 min. LCMS m/z=417 [M+H]⁺, ¹H NMR (300 MHz, DMSO-d₆) δ=9.08 (s, 1H), 8.21 (s, 1H), 7.36 (s, 1H), 7.28 (dd, 2H), 7.22 (dd, 3H), 6.92 (s, 1H), 4.02 (s, 4H), 3.96 (s, 3H), 3.73 (dt, 2H), 3.33-3.27 (m, 2H), 1.52 (m, 2H), 1.33 (m, 2H).

### Example 15:4-(1-(2,2-difluoroethyl)-3-phenyl-1H-pyrazol-4-yl)-6,7-dimethoxyquinazoline

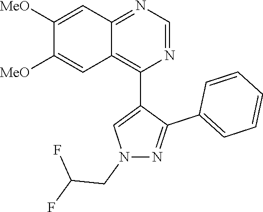 [SMILES: COc1cc2ncnc(-c3cn(CC(F)F)nc3-c3ccccc3)c2cc1OC]

The title compound was obtained as a white solid, 59.8 mg, 33% yield, from 4-chloro-6,7-dimethoxyquinazoline and 1-(2,2-difluoroethyl)-3-phenyl-4-(4,4,5,5-tetramethyl-1,3,2-dioxaborolan-2-yl)-1H-pyrazole (Preparation 41), following a similar procedure to that described in Example 11. LCMS: m/z=397 [M+H]⁺, ¹H NMR (300 MHz, DMSO-d₆) δ=9.07 (s, 1H), 8.40 (s, 1H), 7.36 (s, 1H), 7.28 (m, 5H), 6.90 (s, 1H), 6.55 (t, 1H), 4.91-4.75 (m, 2H), 3.96 (s, 3H), 3.47 (s, 3H).

### Example 16:4-(1-(2,2-difluoroethyl)-3-phenyl-1H-pyrazol-4-yl)-6-methoxy-7-(2-methoxyethoxy)quinazoline

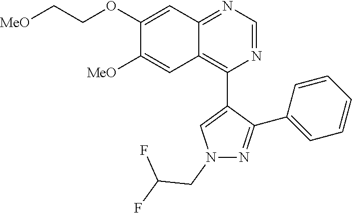 [SMILES: COCCOc1cc2ncnc(-c3cn(CC(F)F)nc3-c3ccccc3)c2cc1OC]

A mixture of Pd(PPh₃)₄ (120 mg, 0.104 mmol), K₃PO₄ (332 mg, 1.56 mmol), 4-chloro-6-methoxy-7-(2-methoxyethoxy)quinazoline (Preparation 130, 280 mg, 1.04 mmol) and 1-(2,2-difluoroethyl)-3-phenyl-4-(4,4,5,5-tetramethyl-1,3,2-dioxaborolan-2-yl)-1H-pyrazole (Preparation 41, 521 mg, 1.56 mmol) in dioxane was stirred at 80° C. for 16 h. The reaction mixture was diluted with EtOAc (120 mL) and washed with water (60 mL). The organic layer was dried over Na₂SO₄, filtered and concentrated under vacuum. The crude product was purified by prep-TLC with DCM:MeOH=10:1. The residue was purified by prep-HPLC (Column: Xselect CSH C18 OBD Column 30*150 mm 5 μm, n: Mobile Phase A: Water (0.05% FA), Mobile Phase B: MeCN: Flow rate: 60 mL/min; Gradient: 33% B isocratic, to give the title compound, 63.5 mg as an off-white solid. LCMS: m/z=441 [M+H]⁺, ¹H NMR (400 MHZ, DMSO-d₆) δ 9.06 (s, 1H), 8.40 (s, 1H), 7.37 (s, 1H), 7.35-7.27 (m, 5H), 6.89 (s, 1H), 6.56 (t, 1H), 4.84 (td, 2H), 4.34-4.27 (m, 2H), 3.76-3.70 (m, 2H), 3.47 (s, 3H), 3.32 (s, 3H).

### Example 17 and 18:6-(((3S,4S)-3-fluorotetrahydro-2H-pyran-4-yl)oxy)-7-methoxy-4-(1-methyl-3-phenyl-1H-pyrazol-4-yl)quinazoline and 6-(((3R,4R)-3-fluorotetrahydro-2H-pyran-4-yl)oxy)-7-methoxy-4-(1-methyl-3-phenyl-1H-pyrazol-4-yl)quinazoline

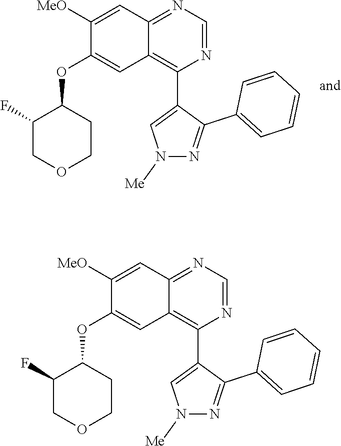 [SMILES: COc1cc2ncnc(-c3cn(C)nc3-c3ccccc3)c2cc1O[C@H]1CCOC[C@@H]1F ; COc1cc2ncnc(-c3cn(C)nc3-c3ccccc3)c2cc1O[C@@H]1CCOC[C@H]1F]

Pd(dppf)Cl₂ (26.9 mg, 35.1 μmol) and K₂CO₃ (72.5 mg, 0.526 mmol) were added to trans-rac-4-chloro-6-(((3S,4S)-3-fluorotetrahydro-2H-pyran-4-yl)oxy)-7-methoxyquinazoline (Preparation 127, 110 mg, 0.351 mmol) and 1-methyl-3-phenyl-4-(4,4,5,5-tetramethyl-1,3,2-dioxaborolan-2-yl)-1H-pyrazole (149 mg, 0.526 mmol) in H₂O (0.5 mL) and dioxane (2 mL) and the reaction mixture was heated to 80° C. for 2 h under N₂. The resulting solution was extracted with EtOAc (3×30 mL), the combined organic layer was dried over Na₂SO₄ and concentrated under vacuum. The crude product was purified by Prep-HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 30% B to 43% B in 8 min: to give trans-rac-6-(((3S,4S)-3-fluorotetrahydro-2H-pyran-4-yl)oxy)-7-methoxy-4-(1-methyl-3-phenyl-1H-pyrazol-4-yl)quinazoline, 50 mg (32%) as a white solid. LCMS m/z=435 [M+H]⁺. This compound (50 mg, 0.115 mmol) was purified by Chiral-HPLC column: Lux 5 μm Cellulose-4, 2.12*25 cm, 5 μm; Mobile Phase A: Hex (0.5% 2M NH₃-MeOH), Mobile Phase B: IPA: Flow rate: 20 mL/min; Gradient: 50% B isocratic; to give

Peak 1, 14.3 mg, 28%, as a white solid, Example 17, 6-(((3S,4S)-3-fluorotetrahydro-2H-pyran-4-yl)oxy)-7-methoxy-4-(1-methyl-3-phenyl-1H-pyrazol-4-yl)quinazoline or 6-(((3R,4R)-3-fluorotetrahydro-2H-pyran-4-yl)oxy)-7-methoxy-4-(1-methyl-3-phenyl-1H-pyrazol-4-yl)quinazoline. LCMS: m/z=435 [M+H]⁺, ¹H NMR (400 MHZ, DMSO-d₆) δ 9.09 (s, 1H), 8.23 (s, 1H), 7.39 (s, 1H), 7.35-7.19 (m, 5H), 7.06 (s, 1H), 4.57-4.36 (m, 1H), 4.19-3.85 (m, 8H), 3.72-3.60 (m, 1H), 3.50-3.36 (m, 2H), 1.68-1.52 (m, 1H), 1.32 (dtd, 1H).

and Peak 2, 6.9 mg, 13% as a white solid, Example 18, 6-(((3R,4R)-3-fluorotetrahydro-2H-pyran-4-yl)oxy)-7-methoxy-4-(1-methyl-3-phenyl-1H-pyrazol-4-yl)quinazoline or 6-(((3S,4S)-3-fluorotetrahydro-2H-pyran-4-yl)oxy)-7-methoxy-4-(1-methyl-3-phenyl-1H-pyrazol-4-yl)quinazoline. LCMS: m/z=435 [M+H]⁺; ¹H NMR (400 MHZ, DMSO-d₆) δ 9.09 (s, 1H), 8.23 (s, 1H), 7.39 (s, 1H), 7.34-7.18 (m, 5H), 7.06 (s, 1H), 4.58-4.39 (m, 1H), 4.17-3.87 (m, 8H), 3.70-3.62 (m, 1H), 3.47-3.35 (m, 2H), 1.66-1.54 (m, 1H), 1.32 (dtd, 1H).

### Example 19:4-(1-(2,2-difluoroethyl)-3-phenyl-1H-pyrazol-4-yl)-7-ethoxy-6-methoxyquinazoline

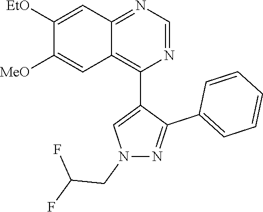 [SMILES: CCOc1cc2ncnc(-c3cn(CC(F)F)nc3-c3ccccc3)c2cc1OC]

Pd(dppf)Cl₂ (32.1 mg, 0.042 mmol) and K₂CO₃ (86.5 mg, 0.627 mmol) were added to 4-chloro-7-ethoxy-6-methoxyquinazoline (Preparation 129, 100 mg, 0.418 mmol) and 1-(2,2-difluoroethyl)-3-phenyl-4-(4,4,5,5-tetramethyl-1,3,2-dioxaborolan-2-yl)-1H-pyrazole (Preparation 41, 209 mg, 0.627 mmol) in H₂O (0.5 mL) and dioxane (2 mL) and the reaction was heated to 80° C. for 2 h under N₂. The resulting solution was extracted with EtOAc (3×30 mL), the combined organic extracts, dried over Na₂SO₄ and concentrated under vacuum. The crude product was purified by Prep-HPLC: Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN: Flow rate: 60 mL/min; Gradient: 37% B to 47% B in 8 min, to give the title compound, 36.2 mg (21%) as a white solid. LCMS: m/z=411 [M+H]⁺, ¹H NMR (400 MHZ, DMSO-d₆) δ 9.06 (s, 1H), 8.39 (s, 1H), 7.40-7.19 (m, 6H), 6.89 (s, 1H), 6.55 (tt, 1H), 4.84 (td, 2H), 4.23 (q, 2H), 3.47 (s, 3H), 1.40 (t, 3H).

### Example 20:4-(1-(2,2-difluoroethyl)-3-phenyl-1H-pyrazol-4-yl)-6,7-dimethoxypyrido[3,2-d]pyrimidine

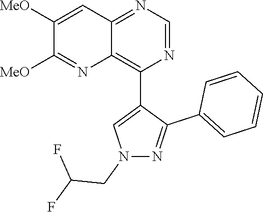 [SMILES: COc1cc2ncnc(-c3cn(CC(F)F)nc3-c3ccccc3)c2nc1OC]

Into a pressure tank reactor purged and maintained with an inert atmosphere of N₂, was placed 4-chloro-6,7-dimethoxypyrido[3,2-d]pyrimidine (Example 7, WO2019148036, 60 mg, 0.266 mmol), 1-(2,2-difluoroethyl)-3-phenyl-4-(4,4,5,5-tetramethyl-1,3,2-dioxaborolan-2-yl)-1H-pyrazole (Preparation 41, 106.6 mg, 0.319 mmol), K₂CO₃ (74.04 mg, 0.532 mmol), water (1.0 mL), Pd(dppf)Cl₂ (19.46 mg, 0.027 mmol) and dioxane (5.0 mL) and the reaction was stirred for 1 h at 80 C. The mixture was extracted with EtOAc (3×10 mL), and the combined organic extracts concentrated under vacuum. The residue was purified by silica gel column eluting with DCM/MeOH (20/1). The product was further purified by prep-HPLC XBridge Prep OBD C18 Column, 30*150 mm, 5 μm; Mobile Phase A: Water (10 mmol/L NH₄HCO₃), Mobile Phase B: MeCN: Flow rate: 60 mL/min; Gradient: 39% B to 49% B in 8 min, to give 14.6 mg, 13.8% of the title compound as a white solid. LCMS: m/z=398 [M+H]⁺. ¹H NMR (300 MHz, DMSO-d₆) δ 8.94 (s, 1H), 8.74 (s, 1H), 7.61 (s, 1H), 7.43 (dd, 2H), 7.35-7.25 (m, 3H), 6.52 (t, 1H), 4.84 (td, 2H), 4.01 (s, 3H), 3.84 (s, 3H).

### Example 21:6-((1,4-dioxepan-6-yl)oxy)-7-methoxy-4-(1-methyl-3-phenyl-1H-pyrazol-4-yl)quinazoline

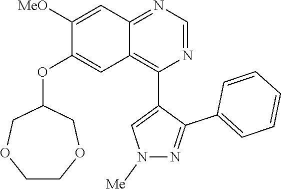 [SMILES: COc1cc2ncnc(-c3cn(C)nc3-c3ccccc3)c2cc1OC1COCCOC1]

To an ice cooled mixture of 1,4-dioxepan-6-ol (Preparation 49, 106 mg, 0.902 mmol) and 7-methoxy-4-(1-methyl-3-phenyl-1H-pyrazol-4-yl)quinazolin-6-ol (Preparation 86, 150 mg, 0.451 mmol) in THF (10 mL) was added PPh₃ (353 mg, 1.35 mmol) and the solution stirred for 10 mins. DIAD (226 mg, 1.12 mmol) was added and the reaction was stirred overnight at 25° C. The reaction mixture was concentrated under vacuum, the resulting solution was diluted with water (20 mL) and the mixture was extracted with EtOAc (2×20 mL). The combined organic layers were washed with brine (20 mL), dried over anhydrous Na₂SO₄ and concentrated under vacuum. The product was purified by prep-TLC with DCM:MeOH (10:1). The residue was then purified by prep-HPLC (Column: YMC-Actus Triart C18, 30 mm×150 mm, 5 um; Mobile Phase A: Water (10 mmol/L NH₄+HCO₃+0.1% NH₃·H₂O), Mobile Phase B: MeCN: Flow rate: 60 mL/min; Gradient: 32% B to 42% B in 8 min, to afford the title compound 49.3 mg, yield: 25.2%. LCMS: m/z=433 [M+H]⁺. ¹H NMR (300 MHz, DMSO-d₆) δ 9.06 (s, 1H), 8.23 (s, 1H), 7.37 (s, 1H), 7.34-7.17 (m, 5H), 6.89 (s, 1H), 4.28 (p, 1H), 4.03 (s, 3H), 3.96 (s, 3H), 3.79-3.53 (m, 8H).

### Example 22: (S)-7-methoxy-4-(1-methyl-3-phenyl-1H-pyrazol-4-yl)-6-(1-(oxetan-3-yl)ethoxy)quinazoline

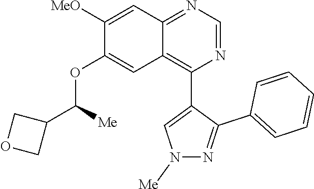 [SMILES: COc1cc2ncnc(-c3cn(C)nc3-c3ccccc3)c2cc1O[C@@H](C)C1COC1]

The title compound was obtained as a white solid, 120.3 mg, 56% yield, from 7-methoxy-4-(1-methyl-3-phenyl-1H-pyrazol-4-yl)quinazolin-6-ol (Preparation 86) and (1R)-1-(oxetan-3-yl) ethan-1-ol, following a similar procedure to that described in Example 21. LCMS: m/z=478.4 [M+H]⁺, ¹H NMR (400 MHz, DMSO-d₆) δ 9.08 (s, 1H), 8.24 (s, 1H), 7.35 (s, 6H), 6.97 (s, 1H), 4.55 (ddd, 2H), 4.30 (t, 2H), 4.23 (t, 1H), 4.18-4.07 (m, 3H), 3.93 (s, 3H), 3.16-3.04 (m, 1H), 1.20 (d, 2H), 0.85 (d, 3H).

### Example 23: 4-(3-((4-(1-(2,2-difluoroethyl)-3-phenyl-1H-pyrazol-4-yl)-7-methoxyquinazolin-6-yl)oxy)propyl)morpholine

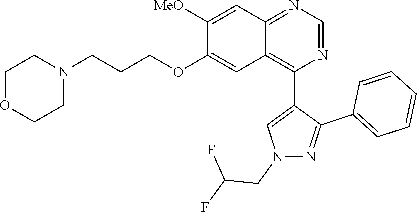 [SMILES: COc1cc2ncnc(-c3cn(CC(F)F)nc3-c3ccccc3)c2cc1OCCCN1CCOCC1]

The title compound was obtained as a white solid, 95.3 mg, 48% yield, from 4-(1-(2,2-difluoroethyl)-3-phenyl-1H-pyrazol-4-yl)-7-methoxyquinazolin-6-ol (Preparation 111) and 3-(morpholin-4-yl) propan-1-ol, following a similar procedure to that described in Example 21, except the crude product was purified by prep-HPLC (XBridge Shield RP18 OBD Column, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 30% B to 40% B in 8 min. LCMS m/z=510 [M+H]⁺. ¹H NMR (400 MHz, DMSO-d₆) δ 9.06 (s, 1H), 8.37 (s, 1H), 7.35 (s, 1H), 7.33-7.20 (m, 5H), 6.88 (s, 1H), 6.55 (t, 1H), 4.83 (td, 2H), 3.96 (s, 3H), 3.63-3.53 (m, 6H), 2.36-2.28 (m, 6H), 1.77 (p, 2H).

### Example 24: 7-ethoxy-4-(1-methyl-3-phenyl-1H-pyrazol-4-yl)-6-((tetrahydro-2H-pyran-4-yl)oxy)quinazoline

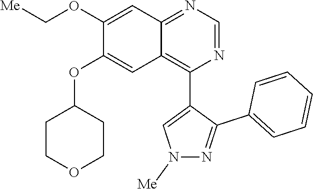 [SMILES: CCOc1cc2ncnc(-c3cn(C)nc3-c3ccccc3)c2cc1OC1CCOCC1]

To an ice-cooled solution of 7-ethoxy-4-(1-methyl-3-phenyl-1H-pyrazol-4-yl)quinazolin-6-ol (Preparation 109, 60 mg, 0.173 mmol) in THF was added PPh₃ (158 mg, 0.606 mmol) and tetrahydro-2H-pyran-4-ol (53.0 mg, 0.519 mmol), followed by DBAD (119 mg, 0.519 mmol) and the reaction stirred at rt for 3 h. The resulting mixture was concentrated under vacuum. The residue was purified by prep-HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 30% B to 42% B in 8 min, to give the title compound (20.7 mg, yield: 27.8%) as a white solid. LCMS: m/z=431 [M+H]⁺. ¹H NMR (400 MHZ, DMSO-d₆) δ 9.07 (s, 1H), 8.21 (s, 1H), 7.34 (s, 1H), 7.28 (dd, 2H), 7.24-7.20 (m, 3H), 6.93 (s, 1H), 4.23 (q, 2H), 4.02 (s, 3H), 3.73 (dt, 2H), 3.35-3.24 (m, 2H), 1.52 (dd, 2H), 1.40 (t, 3H), 1.32 (ddd, 2H).

### Example 25: 7-ethoxy-6-methoxy-4-(1-methyl-3-phenyl-1H-pyrazol-4-yl)quinazoline

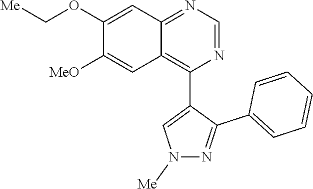 [SMILES: CCOc1cc2ncnc(-c3cn(C)nc3-c3ccccc3)c2cc1OC]

To a solution of 6-methoxy-4-(1-methyl-3-phenyl-1H-pyrazol-4-yl)quinazolin-7-ol (Preparation 131, 270 mg, 0.812 mmol) in DMF was added iodoethane (378 mg, 2.43 mmol) and K₂CO: (335 mg, 2.43 mmol) and the reaction was stirred for 3 h at rt. The resulting solution was diluted with water (50 mL), extracted with EtOAc (2×30 mL) and the organic layers combined. The organic solution was washed with brine (30 mL), dried over anhydrous Na₂SO₄ and concentrated under vacuum. The residue was purified by prep-HPLC (Column: YMC-Actus Triart C18, 30 mm×150 mm, 5 um; Mobile Phase A: Water (20 mmol/L NH₄HCO₃), Mobile Phase B: MeCN: Flow rate: 60 mL/min; Gradient: 30% B to 60% B in 8 min, to give the title compound, 110.5 mg, yield: 37.6% as a white solid. LCMS m/z=361 [M+H]⁺; ¹H NMR (300 MHz, DMSO-d₆) δ 9.04 (s, 1H), 8.28 (s, 1H), 7.31-7.18 (m, 6H), 6.88 (s, 1H), 4.22 (q, 2H), 3.43 (s, 3H), 1.40 (t, 3H).

### Example 26:6,7-diisopropoxy-4-(1-methyl-3-phenyl-1H-pyrazol-4-yl)quinazoline

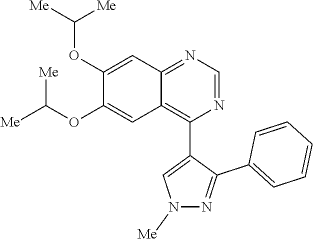 [SMILES: CC(C)Oc1cc2ncnc(-c3cn(C)nc3-c3ccccc3)c2cc1OC(C)C]

To a solution of 4-(1-methyl-3-phenyl-1H-pyrazol-4-yl)quinazoline-6,7-diol (Preparation 132, 50 mg, 0.157 mmol) and KOH (26.44 mg, 0.471 mmol) in DMF (2 mL) was added 2-iodopropane (53.40 mg, 0.314 mmol) and the reaction was stirred at 60° C. for 2 h. The reaction mixture was filtered and the filtrate concentrated in vacuo. The residue was purified by prep-HPLC (column: Phenomenex Gemini-NX C18 75*30 mm*3 um; mobile phase: [water (0.05% NH₃H₂O+10 mM NH₄HCO₃)—MeCN]; B %: 40%-60%, 8 min, to give the title compound (36.7 mg, 58% yield) as a pale yellow solid. LCMS m/z=403 [M+H]⁺ 1H-NMR (400 MHZ, CDCl₃) δ ppm 9.12 (s, 1H), 7.82 (s, 1H), 7.39 (dd, 2H), 7.29 (s, 1H), 7.19-7.17 (m, 3H), 6.95 (s, 1H), 4.77-4.68 (m, 1H), 4.07 (s, 3H), 3.89-3.82 (m, 1H), 1.44 (d, 6H), 1.07 (d, 6H).

### Example 27: 2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole

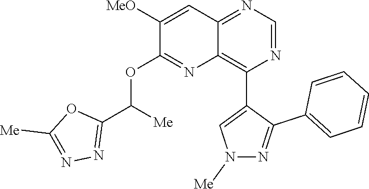 [SMILES: COc1cc2ncnc(-c3cn(C)nc3-c3ccccc3)c2nc1OC(C)c1nnc(C)o1]

A mixture of 6-chloro-7-methoxy-4-(1-methyl-3-phenyl-1H-pyrazol-4-yl)pyrido[3,2-d]pyrimidine (Preparation 242, 50 mg, 0.142 mmol), 1-(5-methyl-1,3,4-oxadiazol-2-yl)ethanol (27.32 mg, 0.213 mmol) and KOtBu (31.90 mg, 0.284 mmol) in THF (5.0 mL) was stirred for 12 h at 80° C. The reaction was quenched by the addition of H₂O, the mixture was extracted with DCM (3×10 mL) and the combined organic extracts were concentrated in vacuo. The residue was purified by silica gel column eluting with DCM/MeOH (20/1). The crude product was further purified by Prep-HPLC Column: YMC-Actus Triart C18 ExRS, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 30% B to 50% B in 8 min, to give the title compound, 16.9 mg (26.8%) as an off-white solid. LCMS: m/z=444 [M+H]⁺ 1H NMR (300 MHz, DMSO-d₆) δ 8.94 (s, 1H), 8.55 (s, 1H), 7.68 (s, 1H), 7.41 (dq, 2H), 7.28 (q, 3H), 6.15 (q, 1H), 4.04 (d, 6H), 2.43 (s, 3H), 1.68 (d, 3H).

### Examples 28 to 33

The compounds in the following table were prepared from 6-chloro-7-methoxy-4-(1-methyl-3-phenyl-1H-pyrazol-4-yl)pyrido[3,2-d]pyrimidine (Preparation 242) and the appropriate alcohol, following a similar procedure to that in Example 27.

| Example | |
|---|---|
| No. | Name, Structure, Starting material (SM), HPLC, Data |
| 28 | 4-(1-((7-methoxy-4-(1-methyl-3-phenyl-1H-pyrazol-4-yl)pyrido |
| | [3,2-d]pyrimidin-6-yl)oxy)ethyl)oxazole |
| | 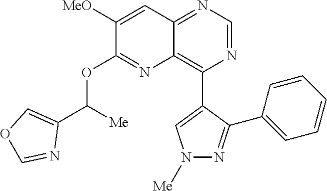 [SMILES: COc1cc2ncnc(-c3cn(C)nc3-c3ccccc3)c2nc1OC(C)c1cocn1] |
| | SM: 1-(1,3-oxazol-4-yl)ethanol |
| | off-white solid, 10.8 mg, 18% yield. LCMS m/z = 429 [M + H]⁺. |
| | 1H NMR (300 MHz, DMSO-d₆) δ 8.94 (s, 1H), 8.47 (s, 1H), 8.36 |
| | (d, 1H), 7.85 (t, 1H), 7.60 (s, 1H), 7.46-7.35 (m, 2H), 7.27 (ddt, |
| | 3H), 5.91 (q, 1H), 4.00 (d, 6H), 1.55 (d, 3H). |
| 29 | 7-methoxy-6-(1-(1-methyl-1H-tetrazol-5-yl)ethoxy)-4-(1-methyl- |
| | 3-phenyl-1H-pyrazol-4-yl)pyrido[3,2-d]pyrimidine |
| | 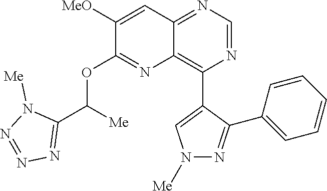 [SMILES: COc1cc2ncnc(-c3cn(C)nc3-c3ccccc3)c2nc1OC(C)c1nnnn1C] |
| | SM: 1-(1-methyl-1,2,3,4-tetrazol-5-yl)ethanol |
| | off-white solid, 7.2 mg, 11% yield. LCMS m/z = 444 [M + H]⁺. |
| | 1H NMR (300 MHz, DMSO-d₆) δ 8.97 (s, 1H), 8.31 (s, 1H), 7.68 |
| | (s, 1H), 7.42-7.32 (m, 2H), 7.26 (dp, 3H), 6.32 (q, 1H), 4.05 (d, |
| | 6H), 3.87 (s, 3H), 1.72 (d, 3H). |
| 30 | 6-(1-(2-fluoropyridin-3-yl)ethoxy)-7-methoxy-4-(1-methyl-3- |
| | phenyl-1H-pyrazol-4-yl)pyrido[3,2-d]pyrimidine |
| | 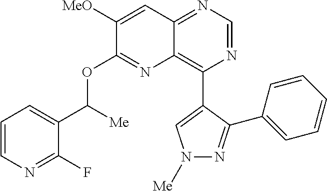 [SMILES: COc1cc2ncnc(-c3cn(C)nc3-c3ccccc3)c2nc1OC(C)c1cccnc1F] |
| | SM: 1-(2-fluoropyridin-3-yl)ethanol |
| | off-white solid, 12 mg, 18.5% yield. LCMS m/z = 457 [M + H]+. |
| | 1H NMR (300 MHz, DMSO-d₆) δ 8.91 (s, 1H), 8.14 (d, 2H), 7.84 |
| | (t, 1H), 7.65 (s, 1H), 7.40-7.28 (m, 2H), 7.27 (dq, 4H), 6.17 (d, |
| | 1H), 4.04 (d, 6H), 1.60 (d, 3H). |
| 31 | 4-(1-((7-methoxy-4-(1-methyl-3-phenyl-1H-pyrazol-4-yl)pyrido |
| | [3,2-d]pyrimidin-6-yl)oxy)ethyl)thiazole |
| | 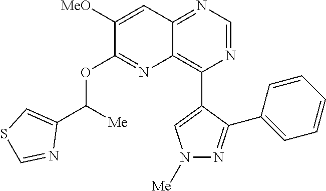 [SMILES: COc1cc2ncnc(-c3cn(C)nc3-c3ccccc3)c2nc1OC(C)c1cscn1] |
| | SM: 1-(thiazol-4-yl)ethan-1-ol |
| | 46.7 mg, 73% yield. 1H NMR (500 MHz, MeOH-d₄) δ 8.91-8.87 |
| | (m, 1H), 8.80 (s, 1H), 8.22 (s, 1H), 7.44 (s, 1H), 7.35-7.29 (m, |
| | 2H), 7.26-7.23 (m, 1H), 7.23-7.14 (m, 3H), 6.06 (q, 1H), 4.00 |
| | (s, 3H), 3.99 (s, 3H), 1.57 (d, 3H). |
| 32 | (R)-2-(1-((7-methoxy-4-(1-methyl-3-phenyl-1H-pyrazol-4- |
| | yl)pyrido[3,2-d]pyrimidin-6-yl)oxy)ethyl)thiazole |
| | 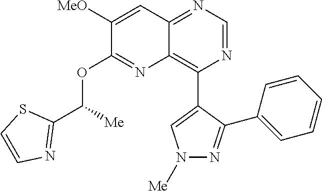 [SMILES: COc1cc2ncnc(-c3cn(C)nc3-c3ccccc3)c2nc1O[C@H](C)c1nccs1] |
| | SM: (1R)-1-(1,3-thiazol-2-yl)ethanol |
| | 11.8 mg, 19% yield, as a light yellow solid. |
| | LCMS m/z = 445 [M + H]⁺ |
| | 1H NMR (300 MHz, DMSO-d₆) δ 8.92 (s, 1H), 8.40 (s, 1H), 7.83 |
| | (d, 1H), 7.72-7.63 (m, 2H), 7.45-7.34 (m, 2H), 7.27 (dt, 3H), |
| | 6.25 (q, 1H), 4.04 (d, 6H), 1.69 (d, 3H). |
| 33 | (S)-4-(1-((7-methoxy-4-(1-methyl-3-phenyl-1H-pyrazol-4- |
| | yl)pyrido[3,2-d]pyrimidin-6-yl)oxy)ethyl)-2-methylthiazole |
| | 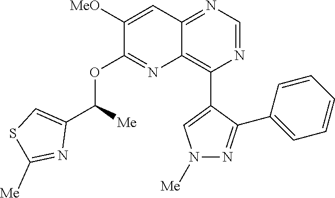 [SMILES: COc1cc2ncnc(-c3cn(C)nc3-c3ccccc3)c2nc1O[C@@H](C)c1csc(C)n1] |
| | SM: (1S)-1-(2-methyl-1,3-thiazol-4-yl) ethan-1-ol |
| | HPLC: XBridge Shield RP18 OBD |
| | Column, 30* 150 mm, 5 μm; Mobile |
| | Phase A: Water (10 mmol/L NH₄HCO₃ + |
| | 0.1%NH₃.H₂O), Mobile Phase |
| | B: MeCN; Flow rate: 60 mL/min; |
| | Gradient: 15% B to 63% B in 10 min |
| | 71.9 mg, 55% yield, an an off-white solid. |
| | LCMS m/z = 459 [M + H]⁺ |
| | 1H NMR (400 MHz, DMSO-d₆) δ 8.90 (s, 1H), 8.43 (s, 1H), 7.60 |
| | (s, 1H), 7.45-7.36 (m, 2H), 7.31-7.23 (m, 3H), 7.21 (d, 1H), 6.11 |
| | (q, 1H), 4.01 (d, 6H), 2.64 (s, 3H), 1.59 (d, 3H). |

### Example 34: 7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine

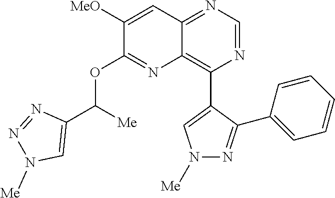 [SMILES: COc1cc2ncnc(-c3cn(C)nc3-c3ccccc3)c2nc1OC(C)c1cn(C)nn1]

To 6-chloro-7-methoxy-4-(1-methyl-3-phenyl-1H-pyrazol-4-yl)pyrido[3,2-d]pyrimidine (Preparation 242, 75 mg, 0.213 mmol) in a 4 dram vial was added a solution of 1-(1-methyl-1H-1,2,3-triazol-4-yl)ethan-1-ol and KOtBu (298 μl, 0.298 mmol) in THF (1 mL) and the reaction heated to 80° C. for 2 h. The reaction mixture was concentrated in vacuo and the crude product purified by reverse phase Isco (X Select CSH C18 OBD Prep column 5 μm, 30 mm×250 mm. 0-40% 0.1% TFA MeCN/Water gradient) to give the title compound, 70 mg, 74% yield. ¹H NMR (MeOH-d₄) δ: 8.91 (s, 1H), 8.44 (d, 1H), 7.67 (d, 1H), 7.52 (s, 1H), 7.45-7.40 (m, 2H), 7.35-7.25 (m, 3H), 6.08 (q, 1H), 4.12 (s, 3H), 4.07 (s, 3H), 4.01 (s, 3H), 1.68 (d, 3H).

### Example 35 and 36: (R)-6-(1-(1-ethyl-1H-1,2,3-triazol-4-yl)ethoxy)-7-methoxy-4-(1-methyl-3-phenyl-1H-pyrazol-4-yl)pyrido[3,2-d]pyrimidine and (S)-6-(1-(1-ethyl-1H-1,2,3-triazol-4-yl) ethoxy)-7-methoxy-4-(1-methyl-3-phenyl-1H-pyrazol-4-yl)pyrido[3,2-d]pyrimidine

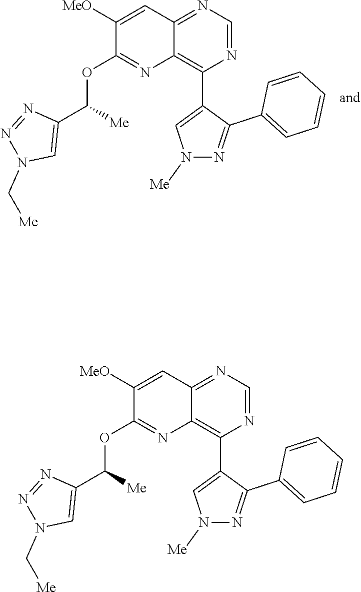 [SMILES: CCn1cc([C@@H](C)Oc2nc3c(-c4cn(C)nc4-c4ccccc4)ncnc3cc2OC)nn1 ; CCn1cc([C@H](C)Oc2nc3c(-c4cn(C)nc4-c4ccccc4)ncnc3cc2OC)nn1]

6-(1-(1-Ethyl-1H-1,2,3-triazol-4-yl)ethoxy)-7-methoxy-4-(1-methyl-3-phenyl-1H-pyrazol-4-yl)pyrido[3,2-d]pyrimidine was obtained, 70 mg, 74% yield, from 6-chloro-7-methoxy-4-(1-methyl-3-phenyl-1H-pyrazol-4-yl)pyrido[3,2-d]pyrimidine (Preparation 242) and 1-(1-ethyl-1H-1,2,3-triazol-4-yl)ethan-1-ol, following a similar procedure to that described in Example 34. This compound, 40 mg, was further purified by CHIRALPAK IE, 2*25 cm, 5 um; Mobile Phase A: Hex:DCM=3:1 (0.5% 2M NH₃-MeOH), Mobile Phase B: IPA; Flow rate: 20 mL/min; Gradient: 15% B isocratic to give

Peak 1, 10.1 mg, as a white solid, Example 35, (R)-6-(1-(1-ethyl-1H-1,2,3-triazol-4-yl)ethoxy)-7-methoxy-4-(1-methyl-3-phenyl-1H-pyrazol-4-yl)pyrido[3,2-d]pyrimidine or (S)-6-(1-(1-ethyl-1H-1,2,3-triazol-4-yl)ethoxy)-7-methoxy-4-(1-methyl-3-phenyl-1H-pyrazol-4-yl)pyrido[3,2-d]pyrimidine. LCMS m/z=457 [M+H]⁺; ¹H NMR (400 MHZ, DMSO-d₆) δ 8.91 (s, 1H), 8.55 (s, 1H), 7.91 (s, 1H), 7.59 (s, 1H), 7.43-7.40 (m, 2H), 7.28-7.26 (m, 3H), 6.11 (q, 1H), 4.31-4.28 (m, 2H), 4.01 (s, 3H), 3.99 (s, 3H), 1.63 (d, 3H), 1.35 (t, 3H).

And Peak 2, 15.5 mg, as a white solid, Example 36, (S)-6-(1-(1-ethyl-1H-1,2,3-triazol-4-yl)ethoxy)-7-methoxy-4-(1-methyl-3-phenyl-1H-pyrazol-4-yl)pyrido[3,2-d]pyrimidine or (R)-6-(1-(1-ethyl-1H-1,2,3-triazol-4-yl)ethoxy)-7-methoxy-4-(1-methyl-3-phenyl-1H-pyrazol-4-yl)pyrido[3,2-d]pyrimidine. LCMS: m/z=457 [M+H]⁺ 1H NMR (400 MHz, DMSO-d₆) δ 8.91 (s, 1H), 8.55 (s, 1H), 7.91 (s, 1H), 7.59 (s, 1H), 7.43-7.40 (m, 2H), 7.28-7.26 (m, 3H), 6.11 (q, 1H), 4.31-4.28 (m, 2H), 4.01 (s, 3H), 3.99 (s, 3H), 1.63 (d, 3H), 1.35 (t, 3H).

### Example 37 and 38: (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole and (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole

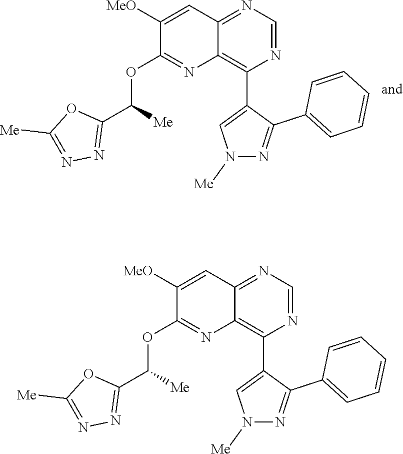 [SMILES: COc1cc2ncnc(-c3cn(C)nc3-c3ccccc3)c2nc1O[C@@H](C)c1nnc(C)o1 ; COc1cc2ncnc(-c3cn(C)nc3-c3ccccc3)c2nc1O[C@H](C)c1nnc(C)o1]

2-(1-((7-Methoxy-4-(1-methyl-3-phenyl-1H-pyrazol-4-yl)pyrido[3,2-d]pyrimidin-6-yl)oxy)ethyl)-5-methyl-1,3,4-oxadiazole (Example 27) was further purified by Column: CHIRALPAK 1H, 2*25 cm, 5 μm; Mobile Phase A: Hex (0.5% 2M NH₃-MeOH), Mobile Phase B: EtOH: Flow rate: 20 mL/min; Gradient: 30% B isocratic: to give

Peak 1, 2.3 mg, 23% as an off-white solid, Example 37, (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole or (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole. LCMS: m/z=444 [M+H]⁺. ¹HNMR (300 MHz, DMSO-d₆) δ 8.96 (s, 1H), 8.58 (s, 1H), 7.58 (q, 1H), 7.46-7.36 (m, 2H), 7.28 (q, 3H), 6.08 (s, 1H), 4.04 (d, 6H), 2.48-2.40 (m, 3H), 1.68 (d, 3H).

Further elution provided Peak 2, 2.3 mg, 23% as an off-white solid, Example 38, (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole or (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-oxadiazole. LCMS: m/z=444 [M+H]⁺. ¹H NMR (300 MHz, DMSO-d₆) δ 8.94 (s, 1H), 8.54 (s, 1H), 7.68 (s, 1H), 7.41 (dd, 2H), 7.32-7.23 (m, 3H), 6.15 (d, 1H), 4.04 (d, 6H), 2.43 (s, 3H), 1.68 (d, 3H).

### Example 39 and 40: (S)-4-(1-((7-methoxy-4-(1-methyl-3-phenyl-1H-pyrazol-4-yl)pyrido[3,2-d]pyrimidin-6-yl)oxy)ethyl) oxazole and (R)-4-(1-((7-methoxy-4-(1-methyl-3-phenyl-1H-pyrazol-4-yl)pyrido[3,2-d]pyrimidin-6-yl)oxy)ethyl) oxazole

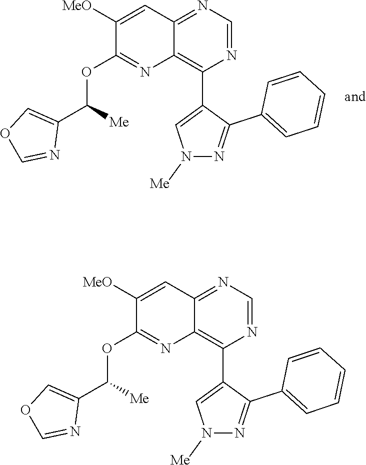 [SMILES: COc1cc2ncnc(-c3cn(C)nc3-c3ccccc3)c2nc1O[C@@H](C)c1cocn1 ; COc1cc2ncnc(-c3cn(C)nc3-c3ccccc3)c2nc1O[C@H](C)c1cocn1]

4-(1-((7-Methoxy-4-(1-methyl-3-phenyl-1H-pyrazol-4-yl)pyrido[3,2-d]pyrimidin-6-yl)oxy)ethyl) oxazole (Example 28), was further purified by prep chiral-HPLC Column: Column: CHIRALPAK IG, 2*25 cm, 5 um; Mobile Phase A: EtOH, Mobile Phase B: Hex (0.5% 2M NH₃—MeOH); Flow rate: 20 mL/min; Gradient: 50% B isocratic, to give

Peak 1, 6.7 mg, 13% yield as an off-white solid, Example 39, (S)-4-(1-((7-methoxy-4-(1-methyl-3-phenyl-1H-pyrazol-4-yl)pyrido[3,2-d]pyrimidin-6-yl)oxy)ethyl) oxazole or (R)-4-(1-((7-methoxy-4-(1-methyl-3-phenyl-1H-pyrazol-4-yl)pyrido[3,2-d]pyrimidin-6-yl)oxy)ethyl) oxazole. LCMS m/z=429 [M+H]⁺. ¹H NMR (300 MHz, DMSO-d₆) δ 8.95 (s, 1H), 8.47 (s, 1H), 8.36 (s, 1H), 7.85 (s, 1H), 7.61 (s, 1H), 7.40 (d, 2H), 7.31-7.23 (m, 3H), 5.92 (d, 1H), 4.00 (d, 6H), 1.55 (d, 3H).

Further elution provided Peak 2, 11 mg, 22.1% as an off-white solid, Example 40, (R)-4-(1-((7-methoxy-4-(1-methyl-3-phenyl-1H-pyrazol-4-yl)pyrido[3,2-d]pyrimidin-6-yl)oxy)ethyl) oxazole or (S)-4-(1-((7-methoxy-4-(1-methyl-3-phenyl-1H-pyrazol-4-yl)pyrido[3,2-d]pyrimidin-6-yl)oxy)ethyl) oxazole. LCMS m/z=429 [M+H]⁺. ¹H NMR (300 MHz, DMSO-d₆) δ 8.95 (s, 1H), 8.47 (s, 1H), 8.37 (s, 1H), 7.85 (s, 1H), 7.61 (s, 1H), 7.41 (s, 2H), 7.31-7.23 (m, 3H), 5.92 (d, 1H), 4.01 (d, 6H), 1.55 (d, 3H).

### Example 41 and 42: (R)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine and (S)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine

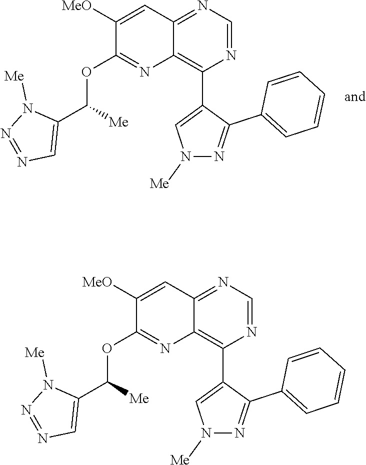 [SMILES: COc1cc2ncnc(-c3cn(C)nc3-c3ccccc3)c2nc1O[C@H](C)c1cnnn1C ; COc1cc2ncnc(-c3cn(C)nc3-c3ccccc3)c2nc1O[C@@H](C)c1cnnn1C]

7-Methoxy-6-(1-(1-methyl-1H-1,2,3-triazol-5-yl)ethoxy)-4-(1-methyl-3-phenyl-1H-pyrazol-4-yl)pyrido[3,2-d]pyrimidine was obtained, 84 mg, from 6-chloro-7-methoxy-4-(1-methyl-3-phenyl-1H-pyrazol-4-yl)pyrido[3,2-d]pyrimidine (Preparation 242) and 1-(1-methyl-1H-1,2,3-triazol-5-yl)ethan-1-ol, following a similar procedure to that described in Example 34. This compound was further purified by Prep-Chiral-HPLC with following condition: Column: CHIRAL ART Amylose-SA, 2*25 cm, 5 um; Mobile Phase A: MtBE (0.5% 2M NH₃—MeOH), Mobile Phase B: MeOH:DCM=1:1: Flow rate: 20 mL/min; Gradient: 10% B isocratic to give

Peak 1, 23 mg, as a yellow solid, Example 41 ((R)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine or (S)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine. LCMS m/z=443 [M+H]⁺, ¹H NMR (400 MHZ, DMSO-d₆) δ 8.98 (s, 1H), 8.33 (s, 1H), 7.73 (s, 1H), 7.64 (s, 1H), 7.38 (dd, 2H), 7.27-7.19 (m, 3H), 6.02 (q, 1H), 4.01 (d, 6H), 3.79 (s, 3H), 1.61 (d, 3H).

Further elution provided Peak 2, 20.3 mg as a white solid, Example 42. (S)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine or (R)-7-methoxy-6-(1-(1-methyl-1H-1,2,3-triazol-5-yl)ethoxy)-4-(1-methyl-3-phenyl-1H-pyrazol-4-yl)pyrido[3,2-d]pyrimidine. LCMS m/z=443 [M+H]⁺, ¹H NMR (400 MHZ, DMSO-d₆) δ 8.98 (s, 1H), 8.33 (s, 1H), 7.73 (s, 1H), 7.64 (s, 1H), 7.38 (dt, 2H), 7.27-7.21 (m, 3H), 6.02 (q, 1H), 4.01 (d, 6H), 3.79 (s, 3H), 1.61 (d, 3H).

### Example 43 and 44: (R)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine and (S)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine

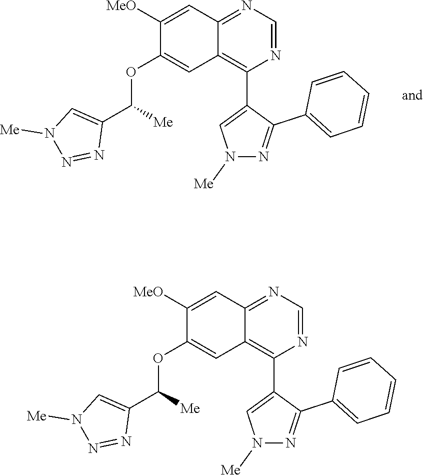 [SMILES: COc1cc2ncnc(-c3cn(C)nc3-c3ccccc3)c2cc1O[C@H](C)c1cn(C)nn1 ; COc1cc2ncnc(-c3cn(C)nc3-c3ccccc3)c2cc1O[C@@H](C)c1cn(C)nn1]

7-Methoxy-6-(1-(1-methyl-1H-1,2,3-triazol-4-yl)ethoxy)-4-(1-methyl-3-phenyl-1H-pyrazol-4-yl)pyrido[3,2-d]pyrimidine (Example 34, 70 mg) was further purified by Column: CHIRAL ART Cellulose-SB, 2*25 cm, 5 μm; Mobile Phase A: Hex (0.5% 2M NH₃—MeOH), Mobile Phase B: EtOH; Flow rate: 20 mL/min; Gradient: 15% B isocratic to give

Peak 1, 5.7 mg, as a white solid, Example 43, (R)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine or (S)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine. LCMS m/z=443 [M+H]⁺. ¹H NMR (400 MHZ, DMSO-d₆) δ 8.91 (s, 1H), 8.55 (s, 1H), 7.86 (s, 1H), 7.59 (s, 1H), 7.42 (dd, 2H), 7.28 (h, 3H), 6.15 (q, 1H), 4.02 (s, 9H), 1.63 (d, 3H).

And Peak 2, 11.2 mg, as a white solid, Example 44, (S)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine or (R)-7-methoxy-6-(1-(1-methyl-1H-1,2,3-triazol-4-yl)ethoxy)-4-(1-methyl-3-phenyl-1H-pyrazol-4-yl)pyrido[3,2-d]pyrimidine. 1H NMR (400 MHZ, DMSO-d₆) δ 8.91 (s, 1H), 8.55 (s, 1H), 7.86 (s, 1H), 7.59 (s, 1H), 7.46-7.37 (m, 2H), 7.27 (p, 3H), 6.15 (q, 1H), 4.05-3.94 (m, 9H), 1.63 (d, 3H).

### Example 45 and 46: (S)-7-methoxy-6-(1-(1-methyl-1H-tetrazol-5-yl)ethoxy)-4-(1-methyl-3-phenyl-1H-pyrazol-4-yl)pyrido[3,2-d]pyrimidine and (R)-7-methoxy-6-(1-(1-methyl-1H-tetrazol-5-yl)ethoxy)-4-(1-methyl-3-phenyl-1H-pyrazol-4-yl)pyrido[3,2-d]pyrimidine

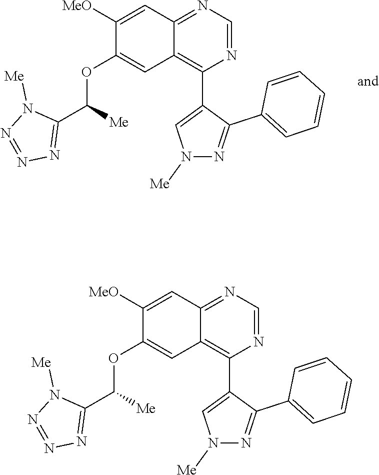 [SMILES: COc1cc2ncnc(-c3cn(C)nc3-c3ccccc3)c2cc1O[C@@H](C)c1nnnn1C ; COc1cc2ncnc(-c3cn(C)nc3-c3ccccc3)c2cc1O[C@H](C)c1nnnn1C]

7-Methoxy-6-(1-(1-methyl-1H-tetrazol-5-yl)ethoxy)-4-(1-methyl-3-phenyl-1H-pyrazol-4-yl)pyrido[3,2-d]pyrimidine (Example 29) was further purified by prep-HPLC Column: CHIRALPAK ID, 2*25 cm, 5 μm; Mobile Phase A: Hex:DCM=3:1 (0.5% 2M NH₃—MeOH), Mobile Phase B: IPA; Flow rate: 20 mL/min; Gradient: 10% B isocratic, to give

Peak 1, 47.8 mg, 32.0% as a light yellow solid, Example 45, (S)-7-methoxy-6-(1-(1-methyl-1H-tetrazol-5-yl)ethoxy)-4-(1-methyl-3-phenyl-1H-pyrazol-4-yl)pyrido[3,2-d]pyrimidine or (R)-7-methoxy-6-(1-(1-methyl-1H-tetrazol-5-yl)ethoxy)-4-(1-methyl-3-phenyl-1H-pyrazol-4-yl)pyrido[3,2-d]pyrimidine. LCMS m/z=444 [M+H]⁺. ¹H NMR (300 MHz, DMSO-d₆) δ 8.97 (s, 1H), 8.31 (s, 1H), 7.68 (s, 1H), 7.38 (dq, 2H), 7.30-7.21 (m, 3H), 6.32 (q, 1H), 4.05 (d, 6H), 3.88 (s, 3H), 1.72 (d, 3H), 1.05 (d, 1H).

And Peak 2, 42.6 mg, 28.5% as an off-white solid, Example 46, (R)-7-methoxy-6-(1-(1-methyl-1H-tetrazol-5-yl)ethoxy)-4-(1-methyl-3-phenyl-1H-pyrazol-4-yl)pyrido[3,2-d]pyrimidine or (S)-7-methoxy-6-(1-(1-methyl-1H-tetrazol-5-yl)ethoxy)-4-(1-methyl-3-phenyl-1H-pyrazol-4-yl)pyrido[3,2-d]pyrimidine. LCMS m/z=444 [M+H]⁺. ¹H NMR (400 MHZ, DMSO-d₆) δ 8.97 (s, 1H), 8.31 (s, 1H), 7.69 (s, 1H), 7.37 (qd, 2H), 7.30-7.22 (m, 3H), 6.32 (q, 1H), 4.05 (d, 6H), 3.88 (s, 3H), 1.72 (d, 3H).

### Example 47 and 48: (S)-6-(1-(2-fluoropyridin-3-yl)ethoxy)-7-methoxy-4-(1-methyl-3-phenyl-1H-pyrazol-4-yl)pyrido[3,2-d]pyrimidine and (R)-6-(1-(2-fluoropyridin-3-yl)ethoxy)-7-methoxy-4-(1-methyl-3-phenyl-1H-pyrazol-4-yl)pyrido[3,2-d]pyrimidine

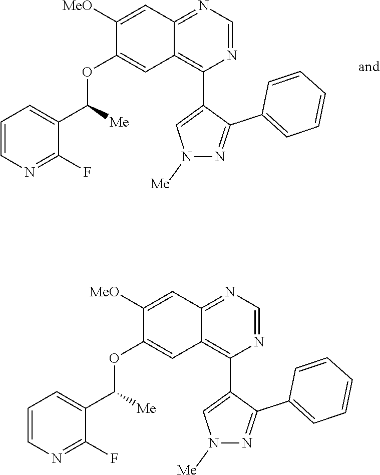 [SMILES: COc1cc2ncnc(-c3cn(C)nc3-c3ccccc3)c2cc1O[C@@H](C)c1cccnc1F ; COc1cc2ncnc(-c3cn(C)nc3-c3ccccc3)c2cc1O[C@H](C)c1cccnc1F]

6-(1-(2-Fluoropyridin-3-yl)ethoxy)-7-methoxy-4-(1-methyl-3-phenyl-1H-pyrazol-4-yl)pyrido[3,2-d]pyrimidine (Example 30) was further purified by HPLC (Column CHIRALPAK AD-H, 2*25 cm, 5 μm; Mobile Phase A: Hex (0.5% 2M NH₃—MeOH), Mobile Phase B: EtOH: Flow rate: 20 mL/min; Gradient: 30% B isocratic, to give

Peak 1, 1.5 mg as an off-white solid, Example 47, (S)-6-(1-(2-fluoropyridin-3-yl)ethoxy)-7-methoxy-4-(1-methyl-3-phenyl-1H-pyrazol-4-yl)pyrido[3,2-d]pyrimidine or (R)-6-(1-(2-fluoropyridin-3-yl)ethoxy)-7-methoxy-4-(1-methyl-3-phenyl-1H-pyrazol-4-yl)pyrido[3,2-d]pyrimidine. LCMS m/z=457 [M+H]⁺; ¹H NMR (300 MHz, DMSO-d₆) δ 8.91 (s, 1H), 8.14 (d, 2H), 7.90-7.77 (m, 1H), 7.65 (s, 1H), 7.36 (dd, 2H), 7.33-7.20 (m, 4H), 6.17 (q, 1H), 4.04 (d, 6H), 1.60 (d, 3H).

Further elution provided Peak 2, 1.3 mg, as an off-white solid, Example 48, (R)-6-(1-(2-fluoro pyridin-3-yl)ethoxy)-7-methoxy-4-(1-methyl-3-phenyl-1H-pyrazol-4-yl)pyrido[3,2-d]pyrimidine or (S)-6-(1-(2-fluoropyridin-3-yl)ethoxy)-7-methoxy-4-(1-methyl-3-phenyl-1H-pyrazol-4-yl)pyrido[3,2-d]pyrimidine. LCMS m/z=457 [M+H]⁺; ¹H NMR (300 MHz, DMSO-d₆) δ 8.91 (s, 1H), 8.14 (d, 2H), 7.90-7.77 (m, 1H), 7.65 (s, 1H), 7.36 (dq, 2H), 7.29 (ddd, 1H), 7.28-7.20 (m, 3H), 6.17 (q, 1H), 4.04 (d, 6H), 1.60 (d, 3H).

### Example 49 and 50: (R)-4-(1-((7-methoxy-4-(1-methyl-3-phenyl-1H-pyrazol-4-yl)pyrido[3,2-d]pyrimidin-6-yl)oxy)ethyl)thiazole and (S)-4-(1-((7-methoxy-4-(1-methyl-3-phenyl-1H-pyrazol-4-yl)pyrido[3,2-d]pyrimidin-6-yl)oxy)ethyl)thiazole

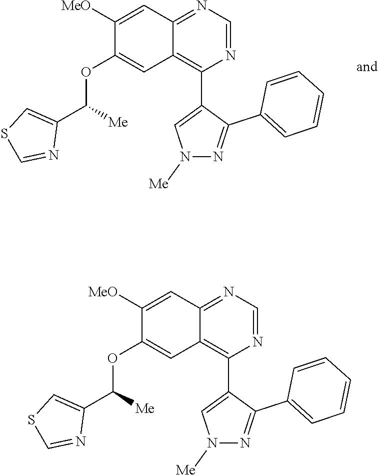 [SMILES: COc1cc2ncnc(-c3cn(C)nc3-c3ccccc3)c2cc1O[C@H](C)c1cscn1 ; COc1cc2ncnc(-c3cn(C)nc3-c3ccccc3)c2cc1O[C@@H](C)c1cscn1]

4-(1-((7-Methoxy-4-(1-methyl-3-phenyl-1H-pyrazol-4-yl)pyrido[3,2-d]pyrimidin-6-yl)oxy)ethyl)thiazole (Example 31, 40 mg) was further purified by Prep-Chiral-HPLC Column: CHIRALPAK IG, 2*25 cm, 5 μm; Mobile Phase A: Hex (0.5% 2M NH₃—MeOH), Mobile Phase B: EtOH: Flow rate: 20 mL/min; Gradient: 30% B isocratic to give

Peak 1, 9.6 mg, as an off-white solid, Example 49 ((R)-4-(1-((7-methoxy-4-(1-methyl-3-phenyl-1H-pyrazol-4-yl)pyrido[3,2-d]pyrimidin-6-yl)oxy)ethyl)thiazole or (S)-4-(1-((7-methoxy-4-(1-methyl-3-phenyl-1H-pyrazol-4-yl)pyrido[3,2-d]pyrimidin-6-yl)oxy)ethyl)thiazole). LCMS m/z=445 [M+H]⁺, ¹H NMR (400 MHZ, DMSO-d₆) δ 9.12 (d, 1H), 8.90 (s, 1H), 8.32 (s, 1H), 7.61 (s, 1H), 7.46 (d, 3H), 7.26 (h, 3H), 6.20 (q, 1H), 4.00 (d, 6H), 1.63 (d, 3H), 1.23 (s, 1H).

Further elution provided Peak 2, 8.7 mg as an off-white solid, Example 50, ((S)-4-(1-((7-methoxy-4-(1-methyl-3-phenyl-1H-pyrazol-4-yl)pyrido[3,2-d]pyrimidin-6-yl)oxy)ethyl)thiazole or (R)-4-(1-((7-methoxy-4-(1-methyl-3-phenyl-1H-pyrazol-4-yl)pyrido[3,2-d]pyrimidin-6-yl)oxy)ethyl)thiazole). LCMS m/z=445 [M+H]⁺, ¹H NMR (400 MHZ, DMSO-d₆) δ 9.12 (d, 1H), 8.90 (s, 1H), 8.32 (s, 1H), 7.61 (s, 1H), 7.46 (dd, 3H), 7.26 (h, 3H), 6.20 (q, 1H), 4.00 (d, 6H), 1.63 (d, 3H), 1.23 (s, 1H).

### Example 51 and 52: (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole and (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole

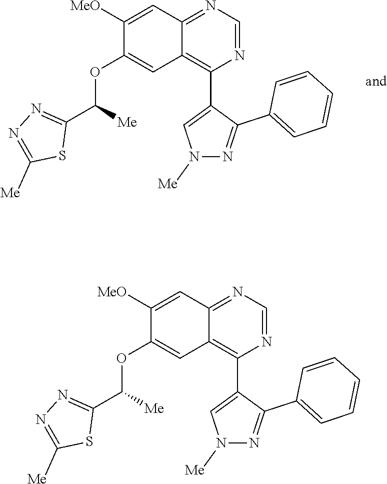 [SMILES: COc1cc2ncnc(-c3cn(C)nc3-c3ccccc3)c2cc1O[C@@H](C)c1nnc(C)s1 ; COc1cc2ncnc(-c3cn(C)nc3-c3ccccc3)c2cc1O[C@H](C)c1nnc(C)s1]

KOtBu (126 mg, 1.13 mmol) was added to 1-(5-methyl-1,3,4-thiadiazol-2-yl)ethan-1-ol (Preparation 51, 162 mg, 1.13 mmol) and 6-chloro-7-methoxy-4-(1-methyl-3-phenyl-1H-pyrazol-4-yl)pyrido[3,2-d]pyrimidine (Preparation 242, 200 mg, 0.568 mmol) in THF (6 mL) and the reaction mixture was stirred at 80° C. for 2 h. The resulting solution was extracted with EtOAc (3×60 mL), the combined organic layer was dried over Na₂SO₄ and concentrated under vacuum. The crude product was purified by Prep-HPLC: Column: YMC-Actus Triart C18 ExRS, 30*150 mm, 5 μm; Mobile Phase A: Water (10 mmol/L NH₄HCO₃+0.1% NH₃·H₂O), Mobile Phase B: MeCN: Flow rate: 60 mL/min; Gradient: 27% B to 49% B in 8 min: to give 2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole (80 mg, yield: 30%) as a white solid. LCMS m/z=460 [M+H]⁺

This compound was further purified by Column: CHIRALPAK AD-H, 2*25 cm, 5 μm; Mobile Phase A: Hex (0.5% 2M NH₃—MeOH), Mobile Phase B: EtOH: Flow rate: 20 mL/min; Gradient: 30% B isocratic: to give Peak 1, 27.4 mg, 34% yield as a white solid, Example 51, (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole or (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole. LCMS m/z=460 [M+H]⁺; ¹H NMR (400 MHz, DMSO-d₆) δ 8.94 (s, 1H), 8.41 (s, 1H), 7.68 (s, 1H), 7.42-7.37 (m, 2H), 7.27 (dt, 3H), 6.25 (q. 1H), 4.05 (d, 6H), 2.64 (s, 3H), 1.72 (d, 3H).

And Peak 2, 27.3 mg, yield: 34%, as a white solid, Example 52, (R)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole or (S)-2-(1-((7-methoxy-4-(1-methyl-3-phenyl-1H-pyrazol-4-yl)pyrido[3,2-d]pyrimidin-6-yl)oxy)ethyl)-5-methyl-1,3,4-thiadiazole. LCMS m/z=460 [M+H]⁺; ¹H NMR (400 MHZ, DMSO-d₆) δ 8.95 (s, 1H), 8.41 (s, 1H), 7.68 (s, 1H), 7.42-7.38 (m, 2H), 7.27 (dt, 3H), 6.25 (q, 1H), 4.05 (d, 6H), 2.64 (s, 3H), 1.72 (d, 3H).

### Example 53 and 54: (R)-4-(1-((7-methoxy-4-(1-methyl-3-phenyl-1H-pyrazol-4-yl)pyrido[3,2-d]pyrimidin-6-yl)oxy)ethyl)-5-methylthiazole and (S)-4-(1-((7-methoxy-4-(1-methyl-3-phenyl-1H-pyrazol-4-yl)pyrido[3,2-d]pyrimidin-6-yl)oxy)ethyl)-5-methylthiazole

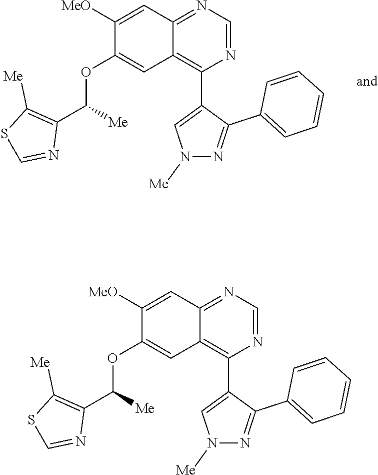 [SMILES: COc1cc2ncnc(-c3cn(C)nc3-c3ccccc3)c2cc1O[C@H](C)c1ncsc1C ; COc1cc2ncnc(-c3cn(C)nc3-c3ccccc3)c2cc1O[C@@H](C)c1ncsc1C]

NaH (69.9 mg, 1.74 mmol) was added to 1-(5-methylthiazol-4-yl)ethan-1-ol (Preparation 50, 250 mg, 1.74 mmol) in THF (8 mL) at 0° C. After stirring for 30 min, 6-chloro-7-methoxy-4-(1-methyl-3-phenyl-1H-pyrazol-4-yl)pyrido[3,2-d]pyrimidine (Preparation 242, 306 mg, 0.870 mmol) was added and the reaction was stirred at 80° C. for 2 h. The reaction was quenched by the addition of water (10 mL), the mixture was extracted with EtOAc and the combined organic extracts concentrated in vacuo. The crude product was purified by prep-TLC with DCM:MeOH=25:1 to give 4-(1-((7-methoxy-4-(1-methyl-3-phenyl-1H-pyrazol-4-yl)pyrido[3,2-d]pyrimidin-6-yl)oxy)ethyl)-5-methylthiazole (50 mg, yield: 12%) as a white solid. LCMS: m/z=459 [M+H]⁺

This was further purified by Prep-Chiral-HPLC as following conditions: Column: CHIRALPAK IE, 2*25 cm, 5 um; Mobile Phase A: Hex:DCM=3:1 (0.5% 2M NH₃—MeOH), Mobile Phase B: IPA: Flow rate: 20 mL/min; Gradient: 10% B isocratic: to give Peak 1, 14.5 mg, 29% as a white solid, Example 53, (R)-4-(1-((7-methoxy-4-(1-methyl-3-phenyl-1H-pyrazol-4-yl)pyrido[3,2-d]pyrimidin-6-yl)oxy)ethyl)-5-methylthiazole or (S)-4-(1-((7-methoxy-4-(1-methyl-3-phenyl-1H-pyrazol-4-yl)pyrido[3,2-d]pyrimidin-6-yl)oxy)ethyl)-5-methylthiazole. LCMS: m/z=459 [M+H]⁺, ¹H NMR (300 MHz, DMSO-d₆) δ 8.95 (s, 1H), 8.80 (s, 1H), 8.39 (s, 1H), 7.56 (s, 1H), 7.38-7.32 (m, 2H), 7.25 (dt, 3H), 6.23 (q, 1H), 4.03 (s, 3H), 3.98 (s, 3H), 2.17 (s, 3H), 1.53 (d, 3H).

And Peak 2, 14 mg, 28% yield as a white solid, Example 54, (S)-4-(1-((7-methoxy-4-(1-methyl-3-phenyl-1H-pyrazol-4-yl)pyrido[3,2-d]pyrimidin-6-yl)oxy)ethyl)-5-methylthiazole or (R)-4-(1-((7-methoxy-4-(1-methyl-3-phenyl-1H-pyrazol-4-yl)pyrido[3,2-d]pyrimidin-6-yl)oxy)ethyl)-5-methylthiazole. LCMS m/z=459 [M+H]⁺; ¹H NMR (300 MHz, DMSO-d₆) δ 8.95 (s, 1H), 8.80 (s, 1H), 8.39 (s, 1H), 7.56 (s, 1H), 7.38-7.33 (m, 2H), 7.28-7.22 (m, 3H), 6.23 (q, 1H), 4.03 (s, 3H), 3.98 (s, 3H), 2.17 (s, 3H), 1.53 (d, 3H).

### Example 55: 7-methoxy-4-(1-methyl-3-phenyl-1H-pyrazol-4-yl)pyrido[3,2-d]pyrimidin-6-yl (S)-2,4-dimethylpiperazine-1-carboxylate

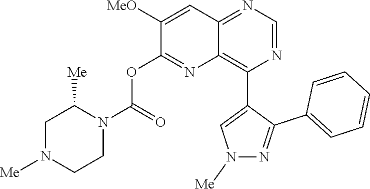 [SMILES: COc1cc2ncnc(-c3cn(C)nc3-c3ccccc3)c2nc1OC(=O)N1CCN(C)C[C@@H]1C]

To a solution of 7-methoxy-4-(1-methyl-3-phenyl-1H-pyrazol-4-yl)pyrido[3,2-d]pyrimidin-6-ol (Preparation 137, 40 mg, 0.120 mmol) and K₂CO₃ (82.92 mg, 0.60 mmol) in DMF (1.00 mL) was added(S)-2,4-dimethylpiperazine-1-carbonyl chloride (Preparation 53, 42.4 mg, 0.240 mmol) and the reaction was stirred at 80° C. for 6 h. The reaction mixture was filtered and the filtrate concentrated in vacuo. The residue was purified by prep-HPLC (column: Waters Xbridge Prep OBD C18 150*40 mm*10 um; mobile phase: [water (0.05% NH₃H₂O+10 mM NH₄HCO₃)—MeCN]; B %: 20%-50% in 8 min, to give the title compound, 13.5 mg, 23.7% yield as a pale yellow solid. LCMS m/z=474 [M+H]⁺. ¹H-NMR (400 MHZ, DMSO-d₆) δ ppm 9.01 (s, 1H), 8.49 (s, 1H), 7.87 (s, 1H), 7.39 (d, 1H), 7.29-7.27 (m, 3H), 4.28-4.21 (m, 1H), 4.02 (d, 6H), 3.88-3.70 (m, 2H), 2.79 (d, 1H), 2.68 (d, 1H), 2.20 (s, 3H), 2.14-2.09 (m, 1H), 1.91 (s, 1H), 1.33 (s, 3H).

### Example 56: 7-methoxy-4-(1-methyl-3-phenyl-1H-pyrazol-4-yl)pyrido[3,2-d]pyrimidin-6-yl (R)-3-methylmorpholine-4-carboxylate

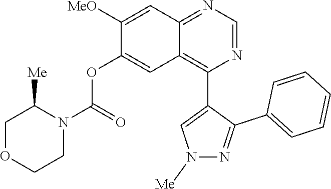 [SMILES: COc1cc2ncnc(-c3cn(C)nc3-c3ccccc3)c2cc1OC(=O)N1CCOC[C@H]1C]

To a solution of 7-methoxy-4-(1-methyl-3-phenyl-1H-pyrazol-4-yl)pyrido[3,2-d]pyrimidin-6-ol (Preparation 137, 30 mg, 0.09 mmol) and (R)-3-methylmorpholine-4-carbonyl chloride (29.45 mg, 0.18 mmol) in DMF (0.5 mL) was added K₂CO₃ (37.32 mg, 0.27 mmol). The mixture was stirred at 80° C. for 12 h under N₂. The reaction mixture was concentrated under reduced pressure and the residue was purified by prep-HPLC (column: Phenomenex Luna C18 75*30 mm*3 μm; mobile phase: [water (0.2% FA)-MeCN]; B %: 15%-45%, 8 min) to afford the title compound, 2.30 mg, yield: 4.96%, FA) as a yellow solid. LCMS m/z=461 [M+H]⁺ 1H NMR (400 MHZ, CDCl₃) δ ppm 9.00 (s, 1H), 8.50 (s, 1H), 7.61 (s, 1H), 7.57-7.51 (m, 2H), 7.34-7.28 (m, 3H), 4.29 (br s, 1H), 4.03 (s, 6H), 3.94 (br s, 2H), 3.74 (br s, 2H), 3.62-3.53 (m, 1H), 3.37 (s, 1H), 1.45 (br s, 3H).

### Example 57:7-methoxy-4-(1-methyl-3-phenyl-1H-pyrazol-4-yl)quinazolin-6-yl 2,4-dimethylpiperazine-1-carboxylate

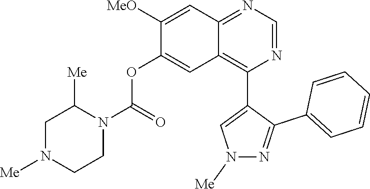 [SMILES: COc1cc2ncnc(-c3cn(C)nc3-c3ccccc3)c2cc1OC(=O)N1CCN(C)CC1C]

To a solution of 7-methoxy-4-(1-methyl-3-phenyl-1H-pyrazol-4-yl)quinazolin-6-ol (Preparation 86, 300 mg, 0.90 mmol) and K₂CO₃ (248 mg, 1.80 mmol) in DMF was added 2,4-dimethylpiperazine-1-carbonyl chloride (238 mg, 1.35 mmol) and the reaction was stirred overnight at rt. The mixture was partitioned between EtOAc and water, the organic layer was dried over Na₂SO₄ and concentrated in vacuo. The crude product was purified by Column: XBridge Shield RP18 OBD Column, 30*150 mm, 5 um; Mobile Phase A: Water (0.05% NH₃H₂O), Mobile Phase B: MeCN: Flow rate: 60 mL/min; Gradient: 42% B to 52% B in 7 min: to give the title compound, 200 mg as an off-white solid. LCMS: m/z=473.4 [M+H]⁺, ¹H NMR (300 MHz, CDCl₃) δ 9.17 (s, 1H), 7.77 (s, 1H), 7.48 (s, 1H), 7.43-7.32 (m, 3H), 7.27-7.13 (m, 3H), 4.31 (s, 1H), 4.05 (s, 3H), 3.98 (s, 3H), 3.90 (d, 1H), 3.29 (s, 1H), 2.79 (d, 1H), 2.70-2.60 (m, 1H), 2.29 (s, 3H), 2.21 (dd, 1H), 2.01 (td, 1H), 1.36 (d, 3H).

### Example 58 and 59:7-methoxy-4-(1-methyl-3-phenyl-1H-pyrazol-4-yl)quinazolin-6-yl (R)-2,4-dimethylpiperazine-1-carboxylate and 7-methoxy-4-(1-methyl-3-phenyl-1H-pyrazol-4-yl)quinazolin-6-yl(S)-2,4-dimethylpiperazine-1-carboxylate

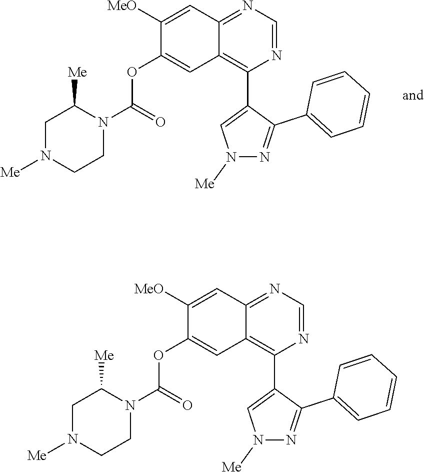 [SMILES: COc1cc2ncnc(-c3cn(C)nc3-c3ccccc3)c2cc1OC(=O)N1CCN(C)C[C@H]1C ; COc1cc2ncnc(-c3cn(C)nc3-c3ccccc3)c2cc1OC(=O)N1CCN(C)C[C@@H]1C]

7-Methoxy-4-(1-methyl-3-phenyl-1H-pyrazol-4-yl)quinazolin-6-yl 2,4-dimethylpiperazine-1-carboxylate (Example 57) was further purified by Prep-HPLC Column: CHIRALPAK AD-H, 2*25 cm, 5 μm; Mobile Phase A: Hex (0.5% 2M NH₃—MeOH), Mobile Phase B: EtOH, Flow rate: 23 mL/min; Gradient: 23% B isocratic, to give

Peak 1, as a light yellow solid, 60 mg, 42%, Example 58, 7-methoxy-4-(1-methyl-3-phenyl-1H-pyrazol-4-yl)quinazolin-6-yl (R)-2,4-dimethylpiperazine-1-carboxylate or 7-methoxy-4-(1-methyl-3-phenyl-1H-pyrazol-4-yl)quinazolin-6-yl(S)-2,4-dimethylpiperazine-1-carboxylate LCMS: m/z=473.2 [M+H]⁺, ¹H NMR (400 MHZ, MeOH-d₄) δ 9.05 (s, 1H), 8.10 (s, 1H), 7.51 (s, 1H), 7.43 (s, 1H), 7.30 (dd, 2H), 7.28-7.17 (m, 3H), 4.30 (s, 1H), 4.07 (s, 3H), 4.01 (s, 3H), 2.83 (d, 1H), 2.74 (d, 1H), 2.29 (s, 3H), 2.22 (dd, 1H), 2.08-1.98 (m, 1H), 1.36 (d, 3H).

Further elution provided Peak 2, as a light yellow solid, 60 mg, 42%, Example 59, 7-methoxy-4-(1-methyl-3-phenyl-1H-pyrazol-4-yl)quinazolin-6-yl(S)-2,4-dimethylpiperazine-1-carboxylate or 7-methoxy-4-(1-methyl-3-phenyl-1H-pyrazol-4-yl)quinazolin-6-yl (R)-2,4-dimethylpiperazine-1-carboxylate. LCMS: m/z=473.2 [M+H]⁺ 1H NMR (400 MHZ, MeOH-d₄) δ 9.05 (s, 1H), 8.09 (s, 1H), 7.51 (s, 1H), 7.43 (s, 1H), 7.30 (dd, 2H), 7.27-7.17 (m, 3H), 4.30 (s, 1H), 4.06 (s, 3H), 4.01 (s, 3H), 2.87-2.79 (m, 1H), 2.74 (d, 1H), 2.29 (s, 3H), 2.22 (dd, 1H), 2.08-1.97 (m, 1H), 1.36 (d, 3H).

### Example 60: 7-methoxy-4-(3-phenyl-1-(2,2,2-trifluoroethyl)-1H-pyrazol-4-yl)quinazolin-6-yl(S)-2,4-dimethylpiperazine-1-carboxylate

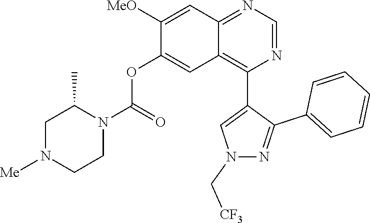 [SMILES: COc1cc2ncnc(-c3cn(CC(F)(F)F)nc3-c3ccccc3)c2cc1OC(=O)N1CCN(C)C[C@@H]1C]

K₂CO₃ (145 mg, 1.049 mmol) and (S)-2,4-dimethylpiperazine-1-carbonyl chloride (Preparation 53, 61.8 mg, 0.350 mmol) were added to a solution of 7-methoxy-4-(3-phenyl-1-(2,2,2-trifluoroethyl)-1H-pyrazol-4-yl)quinazolin-6-ol (Preparation 115, 70 mg, 0.175 mmol) in DMF (2.5 mL) and the reaction was stirred at rt overnight under N₂. The mixture was diluted with water (1 mL) and stirred vigorously. The resulting solution was added to an Isolute HMN SPE tube (5 mL size) and allowed to gravity elute with EtOAc and the filtrate concentrated and dried under vacuum. The residue was purified by silica column chromatography (DCM to 20% MeOH in DCM with NH₄OH) to give the title compound, 44 mg, 46.6% yield. LCMS m/z=305, 307 [M+H]⁺. ¹H NMR (500 MHz, DMSO-d₆) δ 9.69 (s, 1H), 9.19 (s, 1H), 8.41 (s, 1H), 7.58 (s, 1H), 7.54-7.42 (m, 1H), 7.35-7.20 (m, 7H), 5.35 (q, 2H), 4.63-4.10 (m, 3H), 4.01 (s, 3H), 3.60-2.97 (m, 6H), 2.87 (s, 3H), 1.44-1.23 (m, 3H).

### Examples 61 to 70

The compounds in the following table were prepared from the appropriate quinazolin-6-ol, listed below and carbonyl chloride, following a similar procedure to that described in Example 60, (Quinazolin-6-ol) 1: 7-methoxy-4-(1-methyl-3-phenyl-1H-pyrazol-4-yl)quinazolin-6-ol (Preparation 86), (Quinazolin-6-ol) 2: 4-(3-(2-fluorophenyl)-1-methyl-1H-pyrazol-4-yl)-7-methoxyquinazolin-6-ol (Preparation 112), (Quinazolin-6-ol) 3: 4-(3-(2,6-difluorophenyl)-1-methyl-1H-pyrazol-4-yl)-7-methoxyquinazolin-6-ol (Preparation 113), (Quinazolin-6-ol) 4: 7-methoxy-4-(1-(oxetan-3-yl)-3-phenyl-1H-pyrazol-4-yl)quinazolin-6-ol (Preparation 114), (Quinazolin-6-ol) 5: 4-(1-(2,2-difluoroethyl)-3-phenyl-1H-pyrazol-4-yl)-7-methoxyquinazolin-6-ol, (Preparation 111)

| Example | |
|---|---|
| Number | Name, Structure, Starting materials, Data |
| 61 | 7-methoxy-4-(1-methyl-3-phenyl-1H-pyrazol-4-yl)quinazolin-6-yl (R)-3- |
| | methylmorpholine-4-carboxylate |
| | 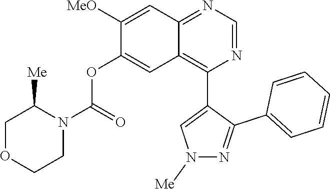 [SMILES: COc1cc2ncnc(-c3cn(C)nc3-c3ccccc3)c2cc1OC(=O)N1CCOC[C@H]1C] |
| | SM: (Quinazolin-6-ol) 1 and (R)-3-methylmorpholine-4-carbonyl chloride |
| | 44 mg. 91% yield as a white solid. LCMS m/z = 460 [M + H]⁺ |
| | 1H NMR (500 MHz, DMSO-d₆) δ 9.13 (d, 1H), 8.25 (s, 1H), 7.61 (d, 1H), |
| | 7.54 (s, 1H), 7.32 (d, 2H), 7.25 (q, 3H), 4.15-3.91 (m, 6H), 3.87 (d, 1H), 3.75- |
| | 3.54 (m, 3H), 3.43-3.30 (m, 3H), 1.30 (s, 3H). |
| 62 | 7-methoxy-4-(1-methyl-3-phenyl-1H-pyrazol-4-yl)quinazolin-6-yl (S)-3- |
| | methylmorpholine-4-carboxylate |
| | 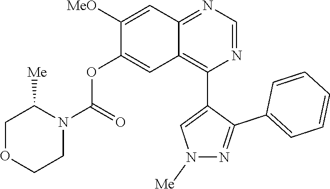 [SMILES: COc1cc2ncnc(-c3cn(C)nc3-c3ccccc3)c2cc1OC(=O)N1CCOC[C@@H]1C] |
| | SM: (Quinazolin-6-ol) 1 and (S)-3-methylmorpholine-4-carbonyl chloride |
| | Yellow solid, 40.2 mg, 58% yield. LCMS m/z = 460 [M + H]⁺ |
| | 1H NMR (500 MHz, DMSO-d₆) δ 9.11 (s, 1H), 8.24 (s, 1H), 7.59 (s, 1H), 7.52 |
| | (s, 1H), 7.29 (dd, 2H), 7.26-7.16 (m, 3H), 4.00 (d, 6H), 3.85 (dd, 1H), 3.65 |
| | (d, 1H), 3.57 (dd, 1H), 3.45-3.36 (m, 4H), 1.32-1.25 (m, 3H). |
| 63 | 7-methoxy-4-(1-methyl-3-phenyl-1H-pyrazol-4-yl)quinazolin-6-yl (3S,5R)- |
| | 3,5-dimethylmorpholine-4-carboxylate |
| | 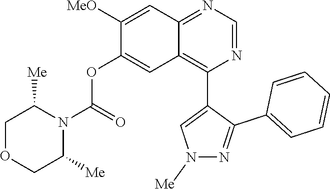 [SMILES: COc1cc2ncnc(-c3cn(C)nc3-c3ccccc3)c2cc1OC(=O)N1[C@H](C)COC[C@@H]1C] |
| | SM: (Quinazolin-6-ol) 1 and (3S,5R)-3,5-dimethylmorpholine-4-carbonyl |
| | chloride (Preparation 54) |
| | 73 mg. 86% yield as a light yellow solid. LCMS m/z = 474 [M + H]⁺. 1H NMR |
| | (500 MHz, DMSO-d₆) δ 9.14 (d, 1H), 8.26 (d, 1H), 7.58 (d, 1H), 7.54 (d, 1H), |
| | 7.31 (dt, 2H), 7.24 (q, 3H), 4.02 (dd, 6H), 3.96 (d, 2H), 3.72 (d, 2H), 3.57 (dt, |
| | 2H), 1.34 (d, 6H). |
| 64 | 7-methoxy-4-(1-methyl-3-phenyl-1H-pyrazol-4-yl)quinazolin-6-yl (3S,5S)- |
| | 3,5-dimethylmorpholine-4-carboxylate |
| | 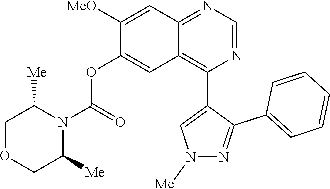 [SMILES: COc1cc2ncnc(-c3cn(C)nc3-c3ccccc3)c2cc1OC(=O)N1[C@@H](C)COC[C@@H]1C] |
| | SM: (Quinazolin-6-ol) 1 and (3S,5S)-3,5-dimethylmorpholine-4-carbonyl |
| | chloride (Preparation 55) |
| | 7.7 mg, 16% yield, as a light yellow solid. LCMS m/z = 474 [M + H]⁺ |
| | 1H NMR (500 MHz, DMSO-d₆) δ 9.13 (d, 1H), 8.25 (d, 1H), 7.52 (s, 2H), |
| | 7.29 (d, 2H), 7.23 (t, 3H), 4.01 (dd, 6H), 3.92-3.72 (m, 4H), 3.46 (s, 2H), |
| | 1.29 (d, 6H). |
| 65 | 7-methoxy-4-(1-methyl-3-phenyl-1H-pyrazol-4-yl)quinazolin-6-yl 7-oxa-4- |
| | azaspiro[2.5]octane-4-carboxylate |
| | 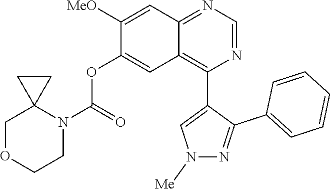 [SMILES: COc1cc2ncnc(-c3cn(C)nc3-c3ccccc3)c2cc1OC(=O)N1CCOCC12CC2] |
| | SM: 7-oxa-4-azaspiro[2.5]octane-4-carbonyl chloride and (Quinazolin-6-ol) 1 |
| | 24 mg, 51% yield as a white solid. LCMS m/z = 472 [M + H]⁺ |
| | 1H NMR (500 MHz, DMSO-d₆) δ 9.12 (d, 1H), 8.24 (d, 1H), 7.57 (d, 1H), |
| | 7.51 (d, 1H), 7.29 (d, 2H), 7.23 (t, 3H), 4.01 (t, 6H), 3.60 (d, 6H), 1.05 (s, 2H), |
| | 0.85 (s, 2H). |
| 66 | 4-(3-(2-fluorophenyl)-1-methyl-1H-pyrazol-4-yl)-7-methoxyquinazolin-6-yl |
| | (S)-2,4-dimethylpiperazine-1-carboxylate |
| | 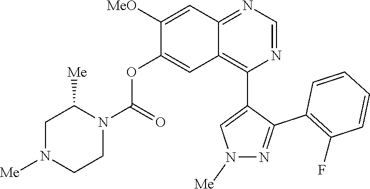 [SMILES: COc1cc2ncnc(-c3cn(C)nc3-c3ccccc3F)c2cc1OC(=O)N1CCN(C)C[C@@H]1C] |
| | SM: (S)-2,4-dimethylpiperazine-1-carbonyl chloride (Preparation 53) and |
| | (Quinazolin-6-ol) 2. |
| | white solid, 35.4 mg, 34% yield. LCMS m/z = 491 [M + H]⁺ 1H NMR (500 |
| | MHz, DMSO-d₆) δ 8.98 (s, 1H), 8.41 (s, 1H), 7.75 (s, 1H), 7.60 (t, 1H), 7.50 |
| | (s, 1H), 7.37 (q, 1H), 7.24 (t, 1H), 7.06-6.91 (m, 1H), 4.25 (s, 1H), 4.06 (s, |
| | 3H), 4.00 (s, 3H), 3.81 (s, 1H), 3.24 (s, 1H), 2.80 (d, 1H), 2.75-2.64 (m, 1H), |
| | 2.23 (s, 3H), 2.12 (d, 1H), 1.92 (s, 1H), 1.33 (s, 3H). |
| 67 | 4-(3-(2,6-difluorophenyl)-1-methyl-1H-pyrazol-4-yl)-7-methoxyquinazolin-6- |
| | yl (S)-3-methylmorpholine-4-carboxylate |
| | 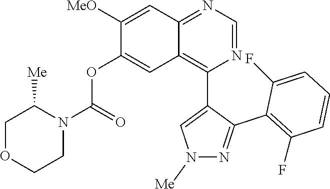 [SMILES: COc1cc2ncnc(-c3cn(C)nc3-c3c(F)cccc3F)c2cc1OC(=O)N1CCOC[C@@H]1C] |
| | SM: (S)-3-methylmorpholine-4-carbonyl chloride and (Quinazolin-6-ol) 3 |
| | Off-white solid, 46.1 mg, 93% yield. LCMS m/z = 496 [M + H]⁺ 1H NMR (500 |
| | MHz, DMSO-d₆) δ 8.92 (d, 1H), 8.52 (s, 1H), 7.84 (s, 1H), 7.49 (s, 1H), 7.42 |
| | (t, 1H), 7.05 (t, 2H), 4.06 (s, 3H), 3.99 (s, 3H), 3.93-3.84 (m, 1H), 3.84- |
| | 3.30 (m, 6H), 1.34 (s, 3H). |
| 68 | 7-methoxy-4-(1-(oxetan-3-yl)-3-phenyl-1H-pyrazol-4-yl)quinazolin-6-yl (S)- |
| | 3-methylmorpholine-4-carboxylate |
| | 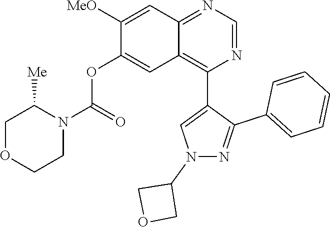 [SMILES: COc1cc2ncnc(-c3cn(C4COC4)nc3-c3ccccc3)c2cc1OC(=O)N1CCOC[C@@H]1C] |
| | SM: (Quinazolin-6-ol) 4 and (S)-3-methylmorpholine-4-carbonyl chloride. |
| | 40.4 mg, 81% yield as a white solid. |
| | LCMS m/z = 502 [M + H]⁺. 1H NMR (500 MHz, DMSO-d₆) δ 9.12 (d, 1H), |
| | 8.40 (t, 1H), 7.63 (d, 1H), 7.53 (d, 1H), 7.35 (dt, 2H), 7.26 (q, 3H), 5.75-5.69 |
| | (m, 1H), 5.08 (dq, 2H), 5.00 (td, 2H), 3.99 (t, 4H), 3.85 (d, 1H), 3.75-3.51 |
| | (m, 2H), 3.41-3.26 (m, 3H), 1.28 (s, 3H). |
| 69 | 7-methoxy-4-(1-(oxetan-3-yl)-3-phenyl-1H-pyrazol-4-yl)quinazolin-6-yl (S)- |
| | 2,4-dimethylpiperazine-1-carboxylate |
| | 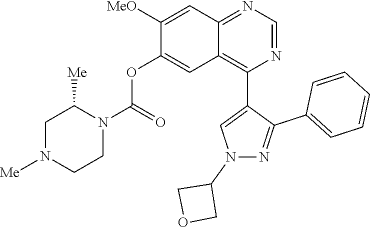 [SMILES: COc1cc2ncnc(-c3cn(C4COC4)nc3-c3ccccc3)c2cc1OC(=O)N1CCN(C)C[C@@H]1C] |
| | SM: (Quinazolin-6-ol) 4 and (S)-2,4-dimethylpiperazine-1-carbonyl chloride |
| | (Preparation 53). |
| | 42.9 mg, 83% yield as an off-white solid. LCMS m/z = 515 [M + H]⁺ |
| | 1H NMR (500 MHz, DMSO-d₆) δ 9.11 (d, 1H), 8.40 (d, 1H), 7.61 (d, 1H), |
| | 7.52 (d, 1H), 7.38-7.32 (m, 2H), 7.26 (q, 3H), 5.74 (s, 1H), 5.08 (t, 2H), 4.20 |
| | (s, 1H), 3.98 (d, 3H), 3.75 (s, 1H), 3.19 (s, 1H), 2.76 (d, 1H), 2.63 (s, 1H), 2.19 |
| | (s, 3H), 2.06 (d, 1H), 1.87 (s, 1H), 1.28 (s, 3H). |
| 70 | 4-(1-(2,2-difluoroethyl)-3-phenyl-1H-pyrazol-4-yl)-7-methoxyquinazolin-6-yl |
| | (S)-2,4-dimethylpiperazine-1-carboxylate |
| | 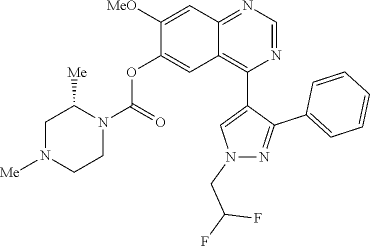 [SMILES: COc1cc2ncnc(-c3cn(CC(F)F)nc3-c3ccccc3)c2cc1OC(=O)N1CCN(C)C[C@@H]1C] |
| | SM: (Quinazolin-6-ol) 5 and (S)-2,4-dimethylpiperazine-1-carbonyl chloride |
| | (Preparation 53). |
| | 26.3 mg, 77% yield as an off-white solid. LCMS m/z = 523 [M + H]⁺ |
| | 1H NMR (500 MHz, DMSO-d₆) δ 9.14 (d, 1H), 8.35 (d, 1H), 7.53 (t, 2H), |
| | 7.31 (s, 1H), 7.26 (t, 3H), 6.55 (dd, 1H), 4.82 (t, 2H), 4.20 (s, 1H), 3.99 (d, |
| | 3H), 3.75 (s, 1H), 3.25 (s, 1H), 2.77 (d, 1H), 2.64 (d, 1H), 2.20 (d, 4H), 2.07 |
| | (d, 1H), 1.88 (s, 1H), 1.28 (s, 3H). |

### Example 71:7-isopropoxy-4-(1-methyl-3-phenyl-1H-pyrazol-4-yl)quinazolin-6-yl (R)-3-methylmorpholine-4-carboxylate

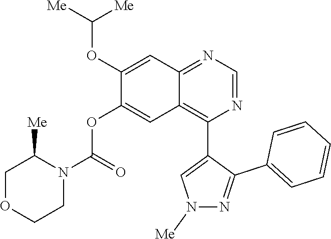 [SMILES: CC(C)Oc1cc2ncnc(-c3cn(C)nc3-c3ccccc3)c2cc1OC(=O)N1CCOC[C@H]1C]

To a solution of (R)-3-methylmorpholine-4-carbonyl chloride (14.53 mg, 0.089 mmol) and 6-bromo-7-isopropoxy-4-(1-methyl-3-phenyl-1H-pyrazol-4-yl)quinazoline (Preparation 102, 16 mg, 0.044 mmol) in DMF (0.50 mL) was added K₂CO₃ (18.41 mg, 0.133 mmol) and the reaction was stirred at 80° C. for 12 h under N₂. The reaction mixture was concentrated under reduced pressure and the residue was purified by prep-HPLC (column: Phenomenex Gemini-NX C18 75*30 mm*3 um; mobile phase: [water (0.05% NH₃H₂O+10 mM NH₄HCO₃)—MeCN]; B %: 25%-60%, 8 min) to give the title compound, 13 mg, 60.1% as yellow solid. LCMS m/z=488 [M+H]⁺ 1H-NMR (400 MHZ, CDCl₃) δ ppm 9.13 (s, 1H), 7.73 (s, 1H), 7.49 (s, 1H), 7.33 (s, 2H), 7.21-7.16 (m, 1H), 7.21-7.16 (m, 3H), 4.75 (spt, 1H), 4.22-4.11 (m, 1H), 4.03 (s, 3H), 3.90 (d, 1H), 3.79 (d, 1H), 3.72-3.61 (m, 2H), 3.49 (dt, 1H), 3.32 (s, 1H), 1.46-1.32 (m, 9H).

### Example 72 to 75

The compounds in the following table were prepared from the appropriate quinazolin-6-ol and carbonyl chloride following a similar procedure to that described in Example 71. (Quinazolin-6-ol) 6: 7-ethoxy-4-(1-methyl-3-phenyl-1H-pyrazol-4-yl)quinazolin-6-ol (Preparation 109)

| Example No | Name, Structure, Starting Materials (SM), Data |
|---|---|
| 72 | 7-methoxy-4-(1-methyl-3-phenyl-1H-pyrazol-4-yl)quinazolin-6-yl (2S,3S)-2,3- |
| | dimethylmorpholine-4-carboxylate |
| | 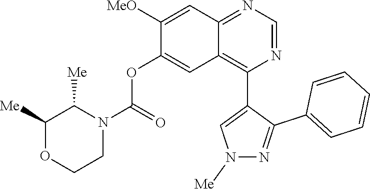 [SMILES: COc1cc2ncnc(-c3cn(C)nc3-c3ccccc3)c2cc1OC(=O)N1CCO[C@@H](C)[C@@H]1C] |
| | SM: (Quinazolin-6-ol) 1 and (2S,3S)-2,3-dimethylmorpholine-4-carbonyl chloride |
| | (Preparation 56) |
| | 26.7 mg, 91.8% yield, was obtained as yellow solid. LCMS m/z = 474 [M + H]⁺ 1H |
| | NMR (400MHz, DMSO-d₆) δ = 9.13 (s, 1H), 8.26 (s, 1H), 7.59 (s, 1H), 7.53 (s, |
| | 1H), 7.33-7.28 (m, 2H), 7.27-7.23 (m, 3H), 4.03 (s, 3H), 4.00 (s, 3H), 3.89-3.62 |
| | (m, 5H), 3.57 (dd, 1H), 1.30 (dd, 6H) |
| 73 | 7-ethoxy-4-(1-methyl-3-phenyl-1H-pyrazol-4-yl)quinazolin-6-yl (R)-2- |
| | methylmorpholine-4-carboxylate |
| | 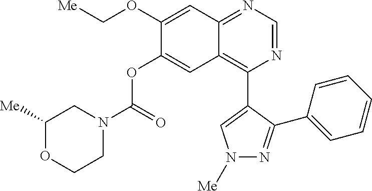 [SMILES: CCOc1cc2ncnc(-c3cn(C)nc3-c3ccccc3)c2cc1OC(=O)N1CCO[C@H](C)C1] |
| | SM: (Quinazolin-6-ol) 6 and (R)-2-methylmorpholine-4-carbonyl chloride |
| | (Preparation 57) |
| | prep-HPLC (column: Phenomenex Gemini-NX C18 75*30 mm*3 um; mobile |
| | phase: [water (0.05%NH₃H₂O + 10 mM NH₄HCO₃)—MeCN]; B%: 20%-45%, 8 min) |
| | 8.10 mg, yield: 19.8%, as a pale yellow solid. LCMS m/z = 474 [M + H]⁺ |
| | 1H-NMR (400 MHz, CDCl₃) δ ppm 9.18 (s, 1H), 7.78 (s, 1H), 7.51 (s, 1H), 7.41- |
| | 7.38 (m, 2H), 7.36 (s, 1H), 7.22-7.20 (m, 3H), 4.23 (q, 2H), 4.06 (s, 3H), 4.01-3.93 |
| | (m, 3H), 3.66-3.60 (m, 2H), 3.18-3.04 (m, 1H), 2.86-2.69 (m, 1H), 1.49 (t, 3H), |
| | 1.22 (d, 3H). |
| 74 | 4-(3-(2-fluorophenyl)-1-methyl-1H-pyrazol-4-yl)-7-methoxyquinazolin-6-yl (S)-3- |
| | methylmorpholine-4-carboxylate |
| | 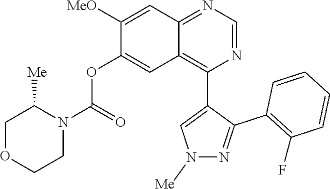 [SMILES: COc1cc2ncnc(-c3cn(C)nc3-c3ccccc3F)c2cc1OC(=O)N1CCOC[C@@H]1C] |
| | SM: (Quinazolin-6-ol) 2 and (S)-3-methylmorpholine-4-carbonyl chloride |
| | prep-HPLC (column: Phenomenex Gemini-NX C18 75*30 mm*3 um; mobile |
| | phase: [water (0.05%NH₃H₂O + 10mM NH₄HCO₃)—MeCN]; B%: 30%-60%, 8 min |
| | 151 mg, 18.3% as yellow solid. LCMS m/z = 478 [M + H]⁺. 1H-NMR (400MHz, |
| | CDCl₃) δ ppm 9.10 (s, 1H), 7.90 (s, 1H), 7.63-7.61 (m, 2H), 7.40 (s, 1H), 7.26-7.25 |
| | (m, 1H), 7.16-7.14 (m, 1H), 6.89-6.86 (m, 1H), 4.21 (d, 1H), 4.10 (s, 3H), 4.00 (s, |
| | 3H), 3.95 (dd, 1H), 3.82 (d, 1H), 3.76-3.72 (m, 2H), 3.59-3.56 (m, 1H), 3.37 (br.s, |
| | 1H), 1.41 (d, 3H). |
| 75^{A} | 7-methoxy-4-(1-methyl-3-phenyl-1H-pyrazol-4-yl)quinazolin-6-yl (S)-4-ethyl-2- |
| | methylpiperazine-1-carboxylate |
| | 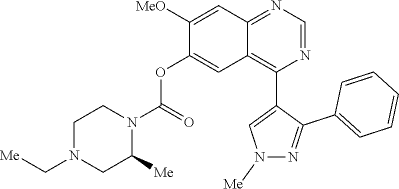 [SMILES: CCN1CCN(C(=O)Oc2cc3c(-c4cn(C)nc4-c4ccccc4)ncnc3cc2OC)[C@@H](C)C1] |
| | SM: (Quinazolin-6-ol) 1 and (S)-4-ethyl-2-methylpiperazine-1-carbonyl chloride |
| | (Preparation 59) |
| | XBridge Prep OBD C18 Column, 30*150 mm, 5 μm; Mobile Phase A: Water |
| | (10mmol/L NH₄HCO₃ + 0.1%NH₃.H₂O), Mobile Phase B: MeCN; Flow rate: 60 |
| | mL/min; Gradient: 30% B to 50% B in 8 min; |
| | 10 mg, 13.7% as a white solid. LCMS: m/z = 487.5 [M + H]⁺. 1H NMR (300 MHz, |
| | DMSO-d₆) δ 9.11 (s, 1H), 8.24 (s, 1H), 7.55 (d, 2H), 7.35-7.27 (m, 2H), 7.24 (q, |
| | 3H), 4.22 (s, 1H), 4.00 (d, 6H), 3.77 (s, 1H), 3.18 (s, 1H), 2.87 (d, 1H), 2.74 (d, |
| | 1H), 2.34 (s, 2H), 2.08 (s, OH), 1.89 (s, 1H), 1.28 (s, 3H), 1.03 (t, 3H). |

^{A} = MeCN was used instead of DMF

### Example 76: 4-(1-(2,2-difluoroethyl)-3-phenyl-1H-pyrazol-4-yl)-7-methoxyquinazolin-6-yl (S)-3-methylmorpholine-4-carboxylate

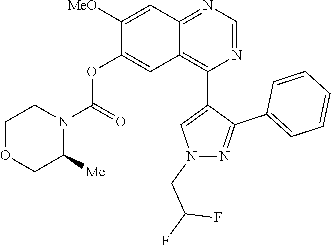 [SMILES: COc1cc2ncnc(-c3cn(CC(F)F)nc3-c3ccccc3)c2cc1OC(=O)N1CCOC[C@@H]1C]

To a solution of 4-(1-(2,2-difluoroethyl)-3-phenyl-1H-pyrazol-4-yl)-7-methoxyquinazolin-6-ol (Preparation 111, 45 mg, 0.118 mmol) in DMF (0.588 mL) under N₂ was added K₂CO₃ (32.5 mg, 0.235 mmol) and (S)-3-methylmorpholine-4-carbonyl chloride (38.5 mg, 0.235 mmol) and the reaction stirred at rt overnight. The mixture was concentrated in vacuo and the residue purified by ISCO Combiflash (0-15% MeOH in DCM) to give an impure product, this was further purified by by Prep-Chiral-HPLC Column: CHIRALPAK IG, 2*25 cm, 5 μm; Mobile Phase A: Hex:DCM=3:1 (0.5% 2M NH₃—MeOH), Mobile Phase B: EtOH: Flow rate: 16 mL/min; Gradient: 50% B isocratic to give 26.1 mg of the title compound as a white solid. LCMS: m/z=510 [M+H]⁺, ¹H NMR (400 MHz, DMSO-d₆) δ 9.14 (s, 1H), 8.35 (s, 1H), 7.54 (s, 2H), 7.36-7.27 (m, 5H), 6.55 (t, 1H), 4.82 (td, 2H), 4.06 (s, 4H), 3.86 (dd, 1H), 3.66 (d, 3H), 3.42 (td, 2H), 1.28 (s, 3H).

### Example 77 to 85

The title compounds were prepared from 7-methoxy-4-(1-methyl-3-phenyl-1H-pyrazol-4-yl)quinazolin-6-ol (Preparation 86) and the appropriate amine (RNH₂) using a library protocol as outlined below.

DIPEA (0.184 mL, 1.05 mmol) was added to a mixture of 7-methoxy-4-(1-methyl-3-phenyl-1H-pyrazol-4-yl)quinazolin-6-ol (Preparation 86, 69.7 mg, 0.21 mmol) and triphosgene (62.4 mg, 0.21 mmol) in DCM (4 mL) with dry-ice cooling and shaking for 1 h. The appropriate amine (0.32 mmol) was added and the reaction mixture was shaken at 30° C. for 2 h. The reaction was filtered and evaporated to dryness by Speedvac. The residue was purified by prep-HPLC (Column: Xtimate C18 150*25 mm*5 μm; mobile phase: A: NH₄OH/H₂O=0.05% v/v; B: MeCN) to afford the title compounds.

| Example Number | Name/Structure/Amine/Data |
|---|---|
| 77 | 7-methoxy-4-(1-methyl-3-phenyl-1H-pyrazol-4-yl)quinazolin-6-yl |
| | 3,3,4-trimethylpiperazine-1-carboxylate |
| | 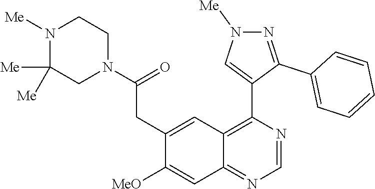 [SMILES: COc1cc2ncnc(-c3cn(C)nc3-c3ccccc3)c2cc1CC(=O)N1CCN(C)C(C)(C)C1] |
| | Amine: 1,2,2-trimethylpiperazine |
| | LCMS: m/z = 487 [M + H]⁺ |
| 78 | 7-methoxy-4-(1-methyl-3-phenyl-1H-pyrazol-4-yl)quinazolin-6-yl |
| | 2,2,4-trimethylpiperazine-1-carboxylate |
| | 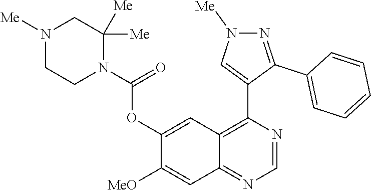 [SMILES: COc1cc2ncnc(-c3cn(C)nc3-c3ccccc3)c2cc1OC(=O)N1CCN(C)CC1(C)C] |
| | Amine: 1,3,3-trimethylpiperazine |
| | LCMS: m/z = 487 [M + H]⁺ |
| 79 | 7-methoxy-4-(1-methyl-3-phenyl-1H-pyrazol-4-yl)quinazolin-6-yl |
| | (2S,6S)-2,4,6-trimethylpiperazine-1-carboxylate |
| | 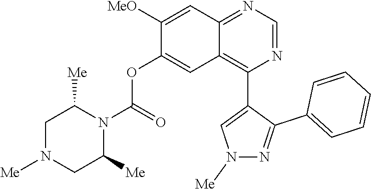 [SMILES: COc1cc2ncnc(-c3cn(C)nc3-c3ccccc3)c2cc1OC(=O)N1[C@@H](C)CN(C)C[C@@H]1C] |
| | Amine: (3S,5S)-1,3,5-trimethylpiperazine |
| | LCMS m/z = 487 [M + H]⁺ |
| 80 | 7-methoxy-4-(1-methyl-3-phenyl-1H-pyrazol-4-yl)quinazolin-6-yl 2,2- |
| | dimethylmorpholine-4-carboxylate |
| | 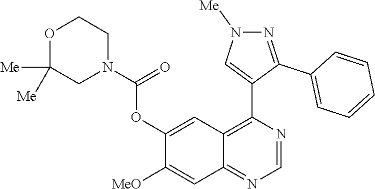 [SMILES: COc1cc2ncnc(-c3cn(C)nc3-c3ccccc3)c2cc1OC(=O)N1CCOC(C)(C)C1] |
| | Amine: 2,2-dimethylmorpholine |
| | LCMS: m/z = 474 [M + H]⁺ |
| 81 | 7-methoxy-4-(1-methyl-3-phenyl-1H-pyrazol-4-yl)quinazolin-6-yl 3,3- |
| | dimethylmorpholine-4-carboxylate |
| | 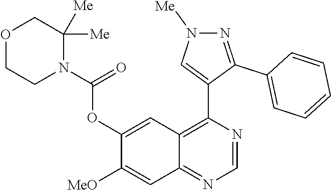 [SMILES: COc1cc2ncnc(-c3cn(C)nc3-c3ccccc3)c2cc1OC(=O)N1CCOCC1(C)C] |
| | Amine: 3,3-dimethylmorpholine |
| | LCMS: m/z = 474 [M + H]⁺ |
| 82 | Cis-rac-7-methoxy-4-(1-methyl-3-phenyl-1H-pyrazol-4-yl)quinazolin-6- |
| | yl (1R,6S)-5-methyl-2,5-diazabicyclo[4.1.0]heptane-2-carboxylate |
| | 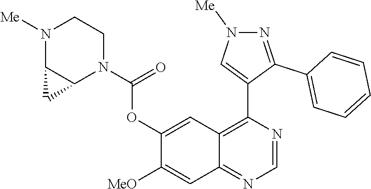 [SMILES: COc1cc2ncnc(-c3cn(C)nc3-c3ccccc3)c2cc1OC(=O)N1CCN(C)[C@H]2C[C@H]21] |
| | Amine: cis-rac-(1S,6R)-2-methyl-2,5-diazabicyclo[4.1.0]heptane |
| | LCMS: m/z = 471 [M + H]⁺ |
| 83 | Cis-rac-7-methoxy-4-(1-methyl-3-phenyl-1H-pyrazol-4-yl)quinazolin-6- |
| | yl (1R,6S)-2-oxa-5-azabicyclo[4.1.0]heptane-5-carboxylate |
| | 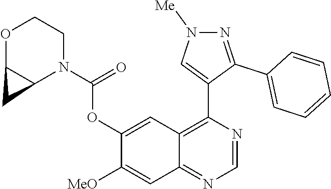 [SMILES: COc1cc2ncnc(-c3cn(C)nc3-c3ccccc3)c2cc1OC(=O)N1CCO[C@@H]2C[C@@H]21] |
| | Amine: cis-rac-(1R,6S)-2-oxa-5-azabicyclo[4.1.0]heptane |
| | LCMS: m/z = 458 [M + H]⁺ |
| 84 | 7-methoxy-4-(1-methyl-3-phenyl-1H-pyrazol-4-yl)quinazolin-6-yl (S)- |
| | hexahydropyrrolo[1,2-a]pyrazine-2(1H)-carboxylate |
| | 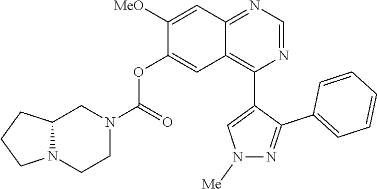 [SMILES: COc1cc2ncnc(-c3cn(C)nc3-c3ccccc3)c2cc1OC(=O)N1CCN2CCC[C@@H]2C1] |
| | Amine: (R)-octahydropyrrolo[1,2-a]pyrazine |
| | LCMS m/z = 485 [M + H]⁺ |
| 85 | 7-methoxy-4-(1-methyl-3-phenyl-1H-pyrazol-4-yl)quinazolin-6-yl (R)- |
| | hexahydropyrrolo[1,2-a]pyrazine-2(1H)-carboxylate |
| | 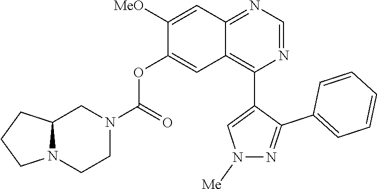 [SMILES: COc1cc2ncnc(-c3cn(C)nc3-c3ccccc3)c2cc1OC(=O)N1CCN2CCC[C@H]2C1] |
| | Amine: (S)-octahydropyrrolo[1,2-a]pyrazine |
| | LCMS m/z = 485 [M + H]⁺ |

### Examples 86 to 88

Part 1: The title compounds were prepared from 7-methoxy-4-(1-methyl-3-phenyl-1H-pyrazol-4-yl)quinazolin-6-ol (Preparation 86) and the appropriate amine (RNH₂) using a library protocol as outlined below.

DIPEA (0.184 mL, 1.05 mmol) was added to a mixture of 7-methoxy-4-(1-methyl-3-phenyl-1H-pyrazol-4-yl)quinazolin-6-ol (Preparation 86, 69.7 mg, 0.21 mmol) and triphosgene (62.4 mg, 0.21 mmol) in DCM (4 mL) with dry-ice cooling and shaking for 1 h. The appropriate amine (0.32 mmol) was added and the reaction mixture was shaken at 30° C. for 2 h. The reaction was filtered and evaporated to dryness by Speedvac.

Part 2: To a solution of the product from Part 1 in DCM (3.0 mL) was added HCl-Dioxane (4 M, 10 eq) and the reaction was stirred at 30° C. for 5 h. The mixture was concentrated under reduced pressure to give a residue. The residue was purified by pre-HPLC Column: Xtimate C18 150*25 mm*5 μm; mobile phase: A: NH₄OH/H₂O=0.05% v/v; B: MeCN, to give the desired product.

| Example No | Name, Structure, Starting Amine, Data |
|---|---|
| 86 | 7-methoxy-4-(1-methyl-3-phenyl-1H-pyrazol-4-yl)quinazolin-6-yl 2,2- |
| | dimethylpiperazine-1-carboxylate |
| | 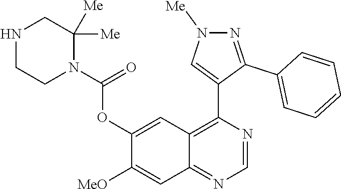 [SMILES: COc1cc2ncnc(-c3cn(C)nc3-c3ccccc3)c2cc1OC(=O)N1CCNCC1(C)C] |
| | Amine: tert-butyl 3,3-dimethylpiperazine-1-carboxylate |
| | LCMS: m/z = 473 [M + H]⁺ |
| 87 | 7-methoxy-4-(1-methyl-3-phenyl-1H-pyrazol-4-yl)quinazolin-6-yl (2R,6S)-2,6- |
| | dimethylpiperazine-1-carboxylate |
| | 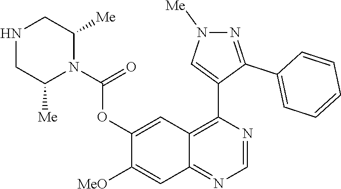 [SMILES: COc1cc2ncnc(-c3cn(C)nc3-c3ccccc3)c2cc1OC(=O)N1[C@H](C)CNC[C@@H]1C] |
| | Amine: tert-butyl 3,5-dimethylpiperazine-1-carboxylate |
| | LCMS: m/z = 473 [M + H]⁺ |
| 88 | 7-methoxy-4-(1-methyl-3-phenyl-1H-pyrazol-4-yl)quinazolin-6-yl (2S,6S)- |
| | 2,6-dimethylpiperazine-1-carboxylate |
| | 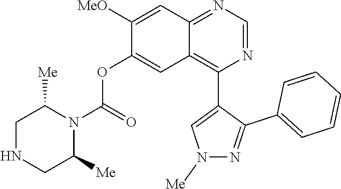 [SMILES: COc1cc2ncnc(-c3cn(C)nc3-c3ccccc3)c2cc1OC(=O)N1[C@@H](C)CNC[C@@H]1C] |
| | Amine: tert-butyl (3S,5S)-3,5-dimethylpiperazine-1-carboxylate |
| | LCMS m/z = 237.2 [M + H]⁺ |

### Example 89:7-methoxy-4-(1-methyl-3-phenyl-1H-pyrazol-4-yl)quinazolin-6-yl (2R,6R)-2,4,6-trimethylpiperazine-1-carboxylate

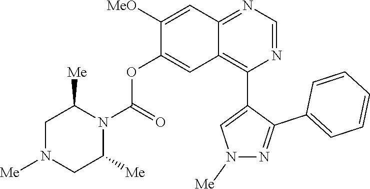 [SMILES: COc1cc2ncnc(-c3cn(C)nc3-c3ccccc3)c2cc1OC(=O)N1[C@H](C)CN(C)C[C@H]1C]

To a solution of 7-methoxy-4-(1-methyl-3-phenyl-1H-pyrazol-4-yl)quinazolin-6-yl (2R,6R)-2,6-dimethylpiperazine-1-carboxylate (Example 88, 100 mg, 0.21 mmol), TEA (152.2 μL, 1.05 mmol) and AcOH (0.3 mL) in MeOH (3 mL) was added formaldehyde (37%, 25.95 mg, 0.32 mmol) and 2-Picoline Borane Complex (66.34 mg, 0.62 mmol). The reaction mixture was shaken at 50° C. for 16 h, then filtered and the solvent removed by Speedvac. The residue was purified by HPLC to give the title compound. LCMS m/z=464 [M+H]⁺

### Example 90:7-methoxy-4-(1-methyl-3-phenyl-1H-pyrazol-4-yl)quinazolin-6-yl (2R,6R)-2,4,6-trimethylpiperazine-1-carboxylate

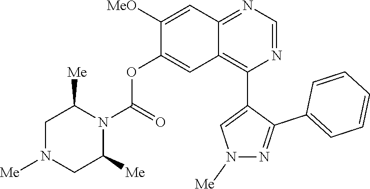 [SMILES: COc1cc2ncnc(-c3cn(C)nc3-c3ccccc3)c2cc1OC(=O)N1[C@H](C)CN(C)C[C@@H]1C]

The title compound was obtained from 7-methoxy-4-(1-methyl-3-phenyl-1H-pyrazol-4-yl)quinazolin-6-yl (2R,6S)-2,6-dimethylpiperazine-1-carboxylate (Example 87) and formaldehyde, following the procedure described in the Example 89. LCMS m/z=464 [M+H]⁺

### Example 91 and 92: trans-rac-7-methoxy-4-(1-methyl-3-phenyl-1H-pyrazol-4-yl)quinazolin-6-yl (2R,5S)-2,5-dimethylmorpholine-4-carboxylate and cis-rac-7-methoxy-4-(1-methyl-3-phenyl-1H-pyrazol-4-yl)quinazolin-6-yl (2S,5S)-2,5-dimethylmorpholine-4-carboxylate

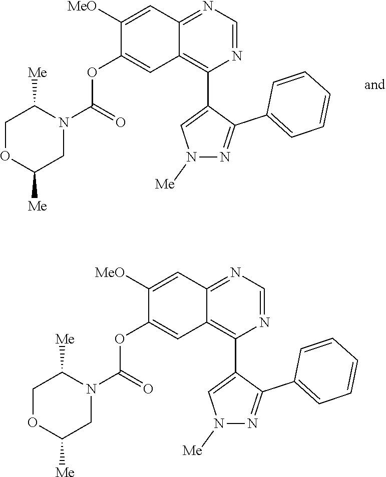 [SMILES: COc1cc2ncnc(-c3cn(C)nc3-c3ccccc3)c2cc1OC(=O)N1C[C@@H](C)OC[C@@H]1C ; COc1cc2ncnc(-c3cn(C)nc3-c3ccccc3)c2cc1OC(=O)N1C[C@H](C)OC[C@@H]1C]

7-Methoxy-4-(1-methyl-3-phenyl-1H-pyrazol-4-yl)quinazolin-6-yl 2,5-dimethylmorpholine-4-carboxylate was obtained, 114.9 mg, from 7-methoxy-4-(1-methyl-3-phenyl-1H-pyrazol-4-yl)quinazolin-6-ol (Preparation 86) and 2,5-dimethylmorpholine following the procedure described for Examples 77 to 85. The compound was further purified by SFC DAICEL CHIRALPAK AD (250 mm*30 mm, 10 μm) 0.1% NH₃H₂O, 30% EtOH, to give peak 1, Example 91, 53 mg as a white solid, trans-rac-7-methoxy-4-(1-methyl-3-phenyl-1H-pyrazol-4-yl)quinazolin-6-yl (2R,5S)-2,5-dimethylmorpholine-4-carboxylate or cis-rac-7-methoxy-4-(1-methyl-3-phenyl-1H-pyrazol-4-yl)quinazolin-6-yl (2S,5S)-2,5-dimethylmorpholine-4-carboxylate. LCMS m/z=474 [M+H]⁺Further elution provided peak 2, Example 92, 62.0 mg, as a white solid, cis-rac-7-methoxy-4-(1-methyl-3-phenyl-1H-pyrazol-4-yl)quinazolin-6-yl (2R,5S)-2,5-dimethylmorpholine-4-carboxylate or trans-rac-7-methoxy-4-(1-methyl-3-phenyl-1H-pyrazol-4-yl)quinazolin-6-yl (2S,5S)-2,5-dimethylmorpholine-4-carboxylate. LCMS m/z=474 [M+H]⁺

### Example 93:7-methoxy-4-(1-methyl-3-phenyl-1H-pyrazol-4-yl)quinazolin-6-yl(S)-2-methyl-4-(methyl-d₃) piperazine-1-carboxylate

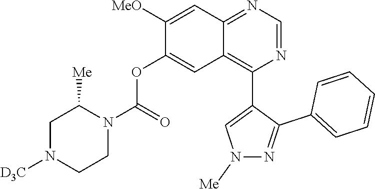 [SMILES: [2H]C([2H])([2H])N1CCN(C(=O)Oc2cc3c(-c4cn(C)nc4-c4ccccc4)ncnc3cc2OC)[C@@H](C)C1]

To a mixture of 7-methoxy-4-(1-methyl-3-phenyl-1H-pyrazol-4-yl)quinazolin-6-yl(S)-2-methylpiperazine-1-carboxylate trifluoroacetate (Preparation 145, 50 mg, 0.109 mmol) and K₂CO₃ (15.07 mg, 0.109 mmol in DMF (2 mL) was added trideuterio(iodo)methane (23.71 mg, 0.164 mmol) and the reaction mixture was stirred at 0° C. for 0.5 h. The reaction was concentrated under vacuum to remove trideuterio(iodo)methane, the mixture was filtered and concentrated under reduced pressure. The residue was purified by prep-HPLC Phenomenex Gemini-NX C18 75*30 mm*3 μm; mobile phase: [water (0.05% NH₃H₂O+10 mM NH₂HCO₃)—MeCN to afford the title compound, 33.70 mg, 65% yield as yellow solid. LCMS m/z=476 [M+H]⁺. ¹H-NMR (400 MHZ, CDCl₃) δ ppm 9.21 (s, 1H), 7.83 (s, 1H), 7.52 (s, 1H), 7.43-7.41 (m, 3H), 7.26-7.24 (m, 3H), 4.36 (br s, 1H), 4.10 (s, 3H), 4.03 (s, 3H), 3.94 (br d, 1H), 3.33 (br s, 1H), 2.83 (br d, 1H), 2.69 (br d, 1H), 2.25 (br dd, 1H), 2.05 (td, 1H), 1.41 (br d, 3H).

### Example 94:7-methoxy-4-(1-methyl-3-phenyl-1H-pyrazol-4-yl)quinazolin-6-yl (2R,3S)-2,3-dimethylmorpholine-4-carboxylate

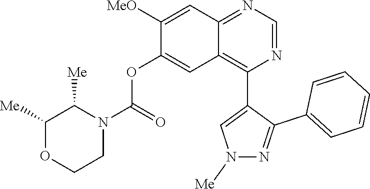 [SMILES: COc1cc2ncnc(-c3cn(C)nc3-c3ccccc3)c2cc1OC(=O)N1CCO[C@H](C)[C@@H]1C]

To a solution of 7-methoxy-4-(1-methyl-3-phenyl-1H-pyrazol-4-yl)quinazolin-6-ol (Preparation 86, 100 mg, 0.301 mmol) and bis(trichloromethyl) carbonate (446.43 mg, 1.50 mmol) in THF (20 mL) was added DIPEA (50.55 mg, 0.391 mmol) dropwise at 0° C. and the reaction was stirred at 25° C. for 12 h. The reaction mixture was concentrated under reduced pressure to give 7-methoxy-4-(1-methyl-3-phenyl-1H-pyrazol-4-yl)quinazolin-6-yl carbonochloridate (100 mg, crude) as a yellow solid. To a mixture of (2R,3S)-2,3-dimethylmorpholine hydrochloride (15 mg, 0.1 mmol) and TEA (20 mg, 0.198 mmol) in DCM (2 mL) was added 7-methoxy-4-(1-methyl-3-phenyl-1H-pyrazol-4-yl)quinazolin-6-yl carbonochloridate (42.96 mg, 0.109 mmol) and the reaction stirred at 20° C. for 1 h. The mixture was concentrated and the residue purified by prep-HPLC column: Phenomenex Gemini-NX C18 75*30 mm*3 um; mobile phase: [water (0.05% NH₃H₂O+10 mM NH₄HCO₃)—MeCN]; B %: 20%-55%, 8 min) to give the title compound (13.4 mg, 26.0% yield) as a pale yellow solid. LCMS m/z=474.2 [M+H]⁺ 1H-NMR (400 MHZ, CDCl₃) δ ppm 9.22 (s, 1H), 7.86 (s, 1H), 7.53 (d, 1H), 7.46 (s, 1H), 7.42 (dd, 2H), 7.26-7.24 (m, 3H), 4.11 (s, 3H), 4.08 (br s, 1H), 4.04 (s, 3H), 4.00-3.94 (m, 1H), 3.85-3.81 (m, 1H), 3.78-3.75 (m, 1H), 3.65 (br t, 1H), 3.40-3.20 (m, 1H), 1.30-1.18 (m, 6H).

### Example 95: 4-(1-ethyl-3-phenyl-1H-pyrazol-4-yl)-7-methoxyquinazolin-6-yl(S)-2,4-dimethylpiperazine-1-carboxylate

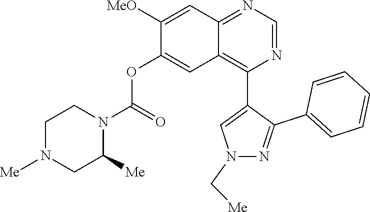 [SMILES: CCn1cc(-c2ncnc3cc(OC)c(OC(=O)N4CCN(C)C[C@@H]4C)cc23)c(-c2ccccc2)n1]

To a solution of 7-methoxy-4-(3-phenyl-1H-pyrazol-4-yl)quinazolin-6-yl(S)-2,4-dimethylpiperazine-1-carboxylate (Preparation 139, 100 mg, 0.218 mmol) in DMF (5 mL) was added NaH (10 mg, 0.436 mmol) at 0° C. and the solution stirred at rt for 1 h. Ethyl iodide (102.04 mg, 0.654 mmol) was added and the reaction stirred at rt for 3 h. The reaction mixture was quenched with water and extracted with DCM. The organic layer was dried over Na₂SO₄, filtered and concentrated to dryness. The residue was purified by silica gel column with 5% MeOH in DCM and further purified by prep-chiral-HPLC Column: CHIRAL ART Cellulose-SC, 2*25 cm, 5 μm; Mobile Phase A: Hex (0.5% 2M NH₃—MeOH), Mobile Phase B: EtOH; Flow rate: 20 mL/min; Gradient: 50% B isocratic, to give the title compound, 14 mg, yield=46.8% as a white solid. LCMS m/z=487 [M+H]^{+ 1}H NMR (300 MHz, DMSO-d₆) δ 9.12 (s, 1H), 8.30 (s, 1H), 7.53 (d, 2H), 7.35-7.27 (m, 2H), 7.24 (q, 3H), 4.31 (q, 2H), 4.19 (s, 1H), 3.99 (s, 3H), 3.75 (s, 1H), 3.15 (s, 1H), 2.79 (s, 1H), 2.64 (d, 1H), 2.20 (s, 3H), 2.07 (d, 1H), 1.86 (d, 1H), 1.52 (t, 3H), 1.28 (d, 3H).

### Example 96: 7-ethoxy-4-(1-methyl-3-phenyl-1H-pyrazol-4-yl)quinazolin-6-yl(S)-2,4-dimethylpiperazine-1-carboxylate

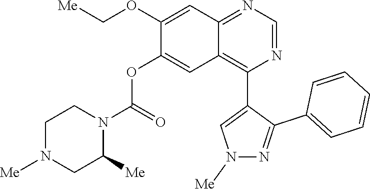 [SMILES: CCOc1cc2ncnc(-c3cn(C)nc3-c3ccccc3)c2cc1OC(=O)N1CCN(C)C[C@@H]1C]

A mixture of 7-bromo-4-(1-methyl-3-phenyl-1H-pyrazol-4-yl)quinazolin-6-yl(S)-2,4-dimethylpiperazine-1-carboxylate (Preparation 153, 104 mg, 0.20 mmol), EtOH (184 mg, 4 mmol), RockphosPd(20 mg, cat) and Cs₂CO₃ (195 mg, 0.60 mmol) in toluene (5 mL) was stirred for 3 h at 95° C. After cooling to rt, the mixture was concentrated. The residue was purified by prep-HPLC Column: XBridge Prep OBD C18 Column, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 30% B to 50% B in 8 min, to give the title compound, 11.4 mg, 11.7% yield, as a white solid. LCMS m/z=487 [M+H]⁺, ¹H NMR (400 MHz, DMSO-d₆): δ 8=9.10 (s, 1H), 8.23 (s, 1H), 7.60 (s, 1H), 7.49 (s, 1H), 7.36-7.09 (m, 5H), 4.25 (s, 3H), 4.02 (s, 3H), 3.79 (s, 1H), 3.33-3.30 (m, 1H), 3.23 (d, 1H), 2.72 (dd, 2H), 2.19 (s, 3H), 2.07 (d, 1H), 1.88 (s, 1H), 1.44-1.08 (m, 6H).

### Example 97:7-methoxy-4-(1-methyl-3-phenyl-1H-pyrazol-4-yl)quinazolin-6-yl (2S,5S)-2,4,5-trimethylpiperazine-1-carboxylate

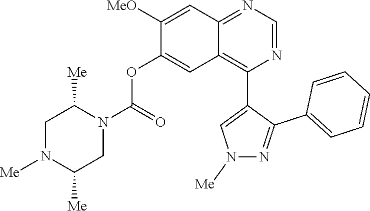 [SMILES: COc1cc2ncnc(-c3cn(C)nc3-c3ccccc3)c2cc1OC(=O)N1C[C@H](C)N(C)C[C@@H]1C]

To a mixture of 7-methoxy-4-(1-methyl-3-phenyl-1H-pyrazol-4-yl)quinazolin-6-yl (2S,5S)-2,5-dimethylpiperazine-1-carboxylate (Preparation 143, 40 mg, 0.085 mmol) in MeOH (2 mL) was added HCHO (13.74 mg, 0.169 mmol, 37.0% purity) in H₂O and the solution stirred at 20° C. for 0.5 h. NaBH₃CN (7.98 mg, 0.127 mmol) was added and the reaction was stirred at 20° C. for 12 h. The mixture was concentrated and the residue was purified by prep-HPLC (column: Phenomenex Gemini-NX C18 75*30 mm*3 μm; mobile phase: [water (0.05% NH₃H₂O+10 mM NH₄HCO₃)—MeCN]; B %: 30%-55%, 8 min) to afford the title compound, 18.80 mg, 45.7% yield as yellow solid. LCMS m/z=487 [M+H]⁺. ¹H-NMR (400 MHZ, CDCl₃) δ ppm 9.21 (s, 1H), 7.82 (s, 1H), 7.52 (s, 1H), 7.43-7.42 (m, 3H), 7.26-7.24 (m, 3H), 4.36-4.33 (m, 1H), 4.10 (s, 3H), 4.03 (s, 3H), 3.88-3.80 (m, 1H), 3.02-2.84 (m, 1H), 2.70 (br d, 1H), 2.45-2.37 (m, 1H), 2.31 (s, 3H), 2.04 (br s, 1H), 1.40 (br dd, 3H), 1.14 (br d, 3H).

### Example 98:7-methoxy-4-(1-methyl-3-phenyl-1H-pyrazol-4-yl)quinazolin-6-yl (2S,3R)-2,3,4-trimethylpiperazine-1-carboxylate

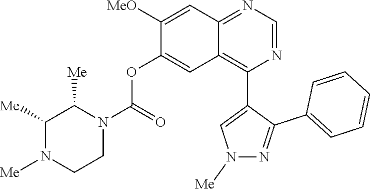 [SMILES: COc1cc2ncnc(-c3cn(C)nc3-c3ccccc3)c2cc1OC(=O)N1CCN(C)[C@H](C)[C@@H]1C]

To a solution of 7-methoxy-4-(1-methyl-3-phenyl-1H-pyrazol-4-yl)quinazolin-6-yl (2S,3R)-2,3-dimethylpiperazine-1-carboxylate (Preparation 144, 30.0 mg, 0.059 mmol) in MeOH (2.0 mL) was added AcOH (17.70 mg, 0.295 mmol) until pH=5. HCHO (5.71 μL, 0.077 mmol, 37% purity, solution in H₂O) was added, the solution was stirred at 25° C. for 1 h, then NaBH₃CN (5.56 mg, 0.088 mmol) was added and the reaction stirred at 25° C. for 1 h. The reaction mixture was quenched by addition of acetone (1 mL) then concentrated under reduced pressure. The residue was purified by prep-HPLC (column: Phenomenex Gemini-NX C18 75*30 mm*3 um; mobile phase: [water (0.05% NH₃H₂O+10 mM NH₄HCO₃)—MeCN]) to afford the title compound, 26.70 mg, 91.8% yield as yellow solid. LCMS m/z=486 [M+H]⁺. ¹H-NMR (400 MHZ, CDCl₃) δ ppm 9.08 (s, 1H), 8.12 (s, 1H), 7.59 (s, 1H), 7.47 (s, 1H), 7.30 (s, 2H), 7.24 (s, 3H), 4.60-4.32 (m, 1H), 4.31-4.13 (m, 1H), 4.08 (s, 3H), 4.03 (s, 3H), 3.61-3.35 (m, 3H), 3.22-3.05 (m, 1H), 1.40-1.29 (m, 6H).

### Example 99: (7-ethoxy-4-(1-methyl-3-phenyl-1H-pyrazol-4-yl)quinazolin-6-yl)methanol

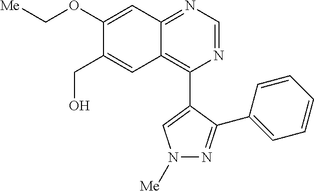 [SMILES: CCOc1cc2ncnc(-c3cn(C)nc3-c3ccccc3)c2cc1CO]

A mixture of 6-bromo-7-ethoxy-4-(1-methyl-3-phenyl-1H-pyrazol-4-yl)quinazoline (Preparation 90, 500 mg, 1.22 mmol), Pd(PPh₃)₂Cl₂ (85.6 mg, 0.122 mmol) and tributylstannyl methanol (587 mg, 1.83 mmol) in dioxane (20 mL) was stirred at 80° C. for 16 h under N₂. The resulting solution was extracted with EtOAc (3×20 mL) the combined organic layer was dried over Na₂SO₄ and concentrated under vacuum. The crude product was purified by prep-TLC with DCM:MeOH=25:1 to afford the title compound (300 mg) as yellow solid. LCMS: m/z=361 [M+H]⁺.

### Example 100: 1-(7-methoxy-4-(1-methyl-3-phenyl-1H-pyrazol-4-yl)quinazolin-6-yl)ethan-1-ol

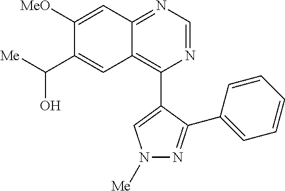 [SMILES: COc1cc2ncnc(-c3cn(C)nc3-c3ccccc3)c2cc1C(C)O]

To a solution of 1-(7-methoxy-4-(1-methyl-3-phenyl-1H-pyrazol-4-yl)quinazolin-6-yl)ethan-1-one (Preparation 172, 130 mg, 0.363 mmol) in MeOH in an ice-bath, was added NaBH₄ (27.5 mg, 0.72 mmol) and the mixture was stirred at rt for 3 h. The solution was quenched with water and concentrated under vacuum. The mixture was diluted with water (20 mL), the resulting solution was extracted with EtOAc (2×20 mL), the organic layers combined, washed with brine (15 mL), dried over Na₂SO₄ and concentrated under vacuum. The residue was purified by prep-HPLC (Column: YMC-Actus Triart C18 ExRS, 30 mm×150 mm, 5 um; Mobile Phase A: Water (10 mmol/L NH₄HCO₃+0.1% NH₃·H₂O), Mobile Phase B: MeCN: Flow rate: 60 mL/min; Gradient: 14% B to 47% B to give the title compound (97.4 mg, yield: 79.4%) as a white solid. LCMS: m/z=361 [M+H]⁺. ¹H NMR (300 MHz, DMSO-d₆) δ 9.09 (s, 1H), 8.21 (s, 1H), 7.91 (d, 1H), 7.34 (s, 1H), 7.30-7.23 (m, 2H), 7.20 (dp, 3H), 5.20-4.81 (m, 2H), 4.04 (s, 3H), 3.98 (s, 3H), 1.06 (d, 3H).

### Example 101 and 102: (S)-1-(4-(1-(2,2-difluoroethyl)-3-phenyl-1H-pyrazol-4-yl)-7-methoxyquinazolin-6-yl)ethan-1-ol and (R)-1-(4-(1-(2,2-difluoroethyl)-3-phenyl-1H-pyrazol-4-yl)-7-methoxyquinazolin-6-yl)ethan-1-ol

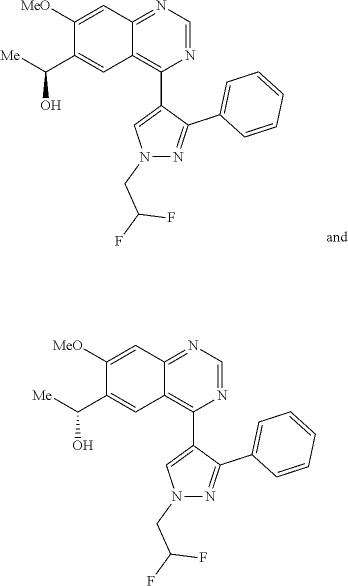 [SMILES: COc1cc2ncnc(-c3cn(CC(F)F)nc3-c3ccccc3)c2cc1[C@H](C)O ; COc1cc2ncnc(-c3cn(CC(F)F)nc3-c3ccccc3)c2cc1[C@@H](C)O]

1-(4-(1-(2,2-Difluoroethyl)-3-phenyl-1H-pyrazol-4-yl)-7-methoxyquinazolin-6-yl)ethan-1-ol was obtained as a white solid, 30 mg, 50% yield, from 1-(4-(1-(2,2-difluoroethyl)-3-phenyl-1H-pyrazol-4-yl)-7-methoxyquinazolin-6-yl)ethan-1-one (Preparation 173), following a similar procedure to that described in Example 100. 1-(4-(1-(2,2-Difluoroethyl)-3-phenyl-1H-pyrazol-4-yl)-7-methoxyquinazolin-6-yl)ethan-1-ol (20 mg) was further purified Column: CHIRAL ART Cellulose-SB, 3*25 cm, 5 μm; Mobile Phase A: Hex (0.5% 2M NH₃—MeOH), Mobile Phase B: IPA: Flow rate: 20 mL/min; Gradient: 30% B isocratic to afford

Peak 1, Example 101, (S)-1-(4-(1-(2,2-difluoroethyl)-3-phenyl-1H-pyrazol-4-yl)-7-methoxyquinazolin-6-yl)ethan-1-ol or (R)-1-(4-(1-(2,2-difluoroethyl)-3-phenyl-1H-pyrazol-4-yl)-7-methoxyquinazolin-6-yl)ethan-1-ol, as a white solid (4 mg, 40%), LCMS: m/z=411 [M+H]⁺; ¹H NMR (300 MHz, DMSO-d₆) δ 10.42 (s, 1H), 8.42 (d, 2H), 8.10 (dd, 2H), 7.76 (dd, 1H), 7.60-7.50 (m, 2H), 7.37 (d, 1H), 5.11-5.01 (m, 1H), 4.17 (d, 3H), 3.90 (s, 3H), 2.86 (dd, 1H), 2.43 (d, J=4.0 Hz, OH), 2.16 (s, 6H), 1.48 (d, 3H).

Further elution provided, Peak 2, 9 mg, 40% as a white solid, Example 102, (R)-1-(4-(1-(2,2-difluoroethyl)-3-phenyl-1H-pyrazol-4-yl)-7-methoxyquinazolin-6-yl)ethan-1-ol or (S)-1-(4-(1-(2,2-difluoroethyl)-3-phenyl-1H-pyrazol-4-yl)-7-methoxyquinazolin-6-yl)ethan-1-ol. LCMS m/z=411 [M+H]⁺. ¹H NMR (400 MHZ, DMSO-d₆) δ 9.11 (s, 1H), 8.31 (s, 1H), 7.88 (d, 1H), 7.35 (s, 1H), 7.27 (ddd, 2H), 7.26-7.18 (m, 3H), 6.56 (t, 1H), 5.06 (d, 1H), 4.99-4.89 (m, 1H), 4.84 (td, 2H), 3.99 (s, 3H), 1.05 (d, 3H).

### Example 103 and 104: (S)-1-(7-ethoxy-4-(1-methyl-3-phenyl-1H-pyrazol-4-yl)quinazolin-6-yl)ethan-1-ol and (R)-1-(7-ethoxy-4-(1-methyl-3-phenyl-1H-pyrazol-4-yl)quinazolin-6-yl)ethan-1-ol

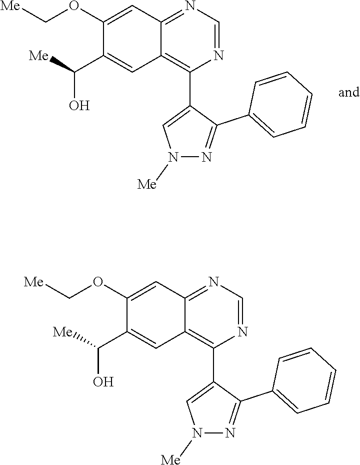 [SMILES: CCOc1cc2ncnc(-c3cn(C)nc3-c3ccccc3)c2cc1[C@H](C)O ; CCOc1cc2ncnc(-c3cn(C)nc3-c3ccccc3)c2cc1[C@@H](C)O]

A mixture of 1-(7-ethoxy-4-(1-methyl-3-phenyl-1H-pyrazol-4-yl)quinazolin-6-yl)ethan-1-one (Preparation 177, 240 mg, 0.593 mmol) and NaBH₄ (74.3 mg, 1.18 mmol) in MeOH (15 mL) was stirred at 25° C. for 2 h. The reaction mixture was quenched with water and then concentrated to dryness. The residue was purified by prep-TLC eluting with DCM:MeOH=20:1. The product was further purified by prep HPLC Column: CHIRALPAK 1H, 5*25 cm, 5 μm; Mobile Phase A: Hex (0.5% 2M NH₃—MeOH), Mobile Phase B: EtOH, Flow rate: 20 mL/min; Gradient: 15% B isocratic, to afford

Peak 1, 32.7 mg, as a white solid, Example 103, (S)-1-(7-ethoxy-4-(1-methyl-3-phenyl-1H-pyrazol-4-yl)quinazolin-6-yl)ethan-1-ol or (R)-1-(7-ethoxy-4-(1-methyl-3-phenyl-1H-pyrazol-4-yl)quinazolin-6-yl)ethan-1-ol. LCMS m/z=375 [M+H]⁺. ¹H NMR (400 MHZ, DMSO-d₆) δ=9.08 (s, 1H), 8.21 (s, 1H), 7.91 (s, 1H), 7.30 (s, 1H), 7.23 (dq, 5H), 5.11-4.87 (m, 2H), 4.34-4.17 (m, 2H), 4.04 (s, 3H), 1.41 (t, 3H), 1.08 (d, 3H).

Further elution provided Peak 2, 31.2 mg, as a white solid, Example 104, (R)-1-(7-ethoxy-4-(1-methyl-3-phenyl-1H-pyrazol-4-yl)quinazolin-6-yl)ethan-1-ol or (S)-1-(7-ethoxy-4-(1-methyl-3-phenyl-1H-pyrazol-4-yl)quinazolin-6-yl)ethan-1-ol. LCMS m/z=375 [M+H]⁺. ¹H NMR (400 MHZ, DMSO-d₆) δ=9.08 (s, 1H), 8.21 (s, 1H), 7.90 (d, 1H), 7.30 (s, 1H), 7.25 (ddd, 2H), 7.20 (q, 3H), 4.94 (q, 2H), 4.32-4.19 (m, 2H), 4.04 (s, 3H), 1.41 (t, 3H), 1.08 (d, 3H).

### Example 105 and 106: (S)-1-(4-(1-ethyl-3-phenyl-1H-pyrazol-4-yl)-7-methoxyquinazolin-6-yl)ethan-1-ol and (R)-1-(4-(1-ethyl-3-phenyl-1H-pyrazol-4-yl)-7-methoxyquinazolin-6-yl)ethan-1-ol

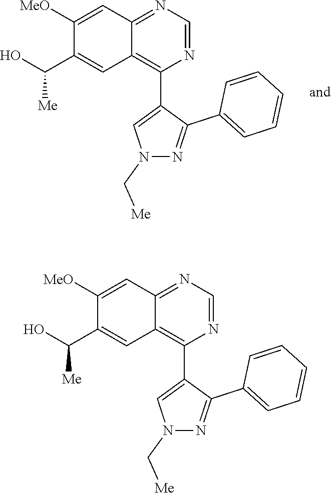 [SMILES: CCn1cc(-c2ncnc3cc(OC)c([C@H](C)O)cc23)c(-c2ccccc2)n1 ; CCn1cc(-c2ncnc3cc(OC)c([C@@H](C)O)cc23)c(-c2ccccc2)n1]

1-(4-(1-Ethyl-3-phenyl-1H-pyrazol-4-yl)-7-methoxyquinazolin-6-yl)ethan-1-ol was obtained from 1-(4-(1-ethyl-3-phenyl-1H-pyrazol-4-yl)-7-methoxyquinazolin-6-yl)ethan-1-one (Preparation 179), following the procedure described in Examples 103 and 104. The racemic product was further purified by prep-CHIRAL-HPLC, Column: CHIRAL ART Cellulose-SB, 2*25 cm, 5 um; Mobile Phase A: Hex (0.5% 2M NH₃/MeOH), Mobile Phase B: EtOH: Flow rate: 20 mL/min; Gradient: 20% B isocratic, to give

Peak 1, 6.9 mg, as a white solid, Example 105, (S)-1-(4-(1-ethyl-3-phenyl-1H-pyrazol-4-yl)-7-methoxyquinazolin-6-yl)ethan-1-ol or (R)-1-(4-(1-ethyl-3-phenyl-1H-pyrazol-4-yl)-7-methoxyquinazolin-6-yl)ethan-1-ol. LCMS: m/z=375 [M+H]⁺; ¹H NMR (400 MHZ, DMSO-d₆): δ=9.09 (s, 1H), 8.26 (s, 1H), 7.90 (d, 1H), 7.33 (s, 1H), 7.31-7.22 (m, 2H), 7.20 (m, 3H), 5.07 (d, 1H), 4.98-4.88 (m, 1H), 4.33 (q, 2H), 3.98 (s, 3H), 1.53 (t, 3H), 1.05 (d, 3H).

Further elution provided Peak 2, 6.5 mg as a light blue solid, Example 106, (R)-1-(4-(1-ethyl-3-phenyl-1H-pyrazol-4-yl)-7-methoxyquinazolin-6-yl)ethan-1-ol or (S)-1-(4-(1-ethyl-3-phenyl-1H-pyrazol-4-yl)-7-methoxyquinazolin-6-yl)ethan-1-ol, LCMS: m/z=375 [M+H]⁺; ¹H NMR (400 MHZ, DMSO-d₆): δ=9.09 (s, 1H), 8.26 (s, 1H), 7.90 (d, 1H), 7.33 (s, 1H), 7.31-7.22 (m, 2H), 7.20 (m, 3H), 5.07 (d, 1H), 4.98-4.88 (m, 1H), 4.33 (q, 2H), 3.98 (s, 3H), 1.53 (t, 3H), 1.05 (d, 3H).

### Example 107 and 108: (S)-1-(7-ethoxy-4-(1-ethyl-3-phenyl-1H-pyrazol-4-yl)quinazolin-6-yl)ethan-1-ol and (R)-1-(7-ethoxy-4-(1-ethyl-3-phenyl-1H-pyrazol-4-yl)quinazolin-6-yl)ethan-1-ol

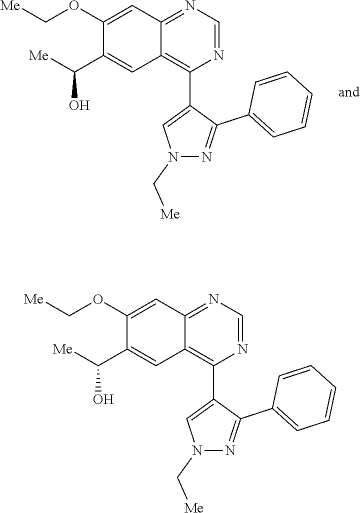 [SMILES: CCOc1cc2ncnc(-c3cn(CC)nc3-c3ccccc3)c2cc1[C@H](C)O ; CCOc1cc2ncnc(-c3cn(CC)nc3-c3ccccc3)c2cc1[C@@H](C)O]

To a solution of 1-(7-ethoxy-4-(1-ethyl-3-phenyl-1H-pyrazol-4-yl)quinazolin-6-yl)ethan-1-one (Preparation 178, 100 mg, 0.258 mmol) in MeOH (3 mL) was added NaBH₄ (19.5 mg, 0.516 mmol) and the reaction stirred at rt for 1 h. The reaction was quenched with water and then concentrated to dryness. The residue was purified by prep-TLC with DCM:MeOH=25:1. The product was further purified by prep-HPLC Column: Lux 5 μm Cellulose-2, 2.12*25 cm, 5 um; Mobile Phase A: Hex (0.5% 2M NH₃—MeOH), Mobile Phase B: EtOH: Flow rate: 20 mL/min; Gradient: 10% B isocratic to give

Peak 1, 10.9 mg, as a light-yellow solid, Example 107, (S)-1-(7-ethoxy-4-(1-ethyl-3-phenyl-1H-pyrazol-4-yl)quinazolin-6-yl)ethan-1-ol or (R)-1-(7-ethoxy-4-(1-ethyl-3-phenyl-1H-pyrazol-4-yl)quinazolin-6-yl)ethan-1-ol. LCMS: m/z=389 [M+H]⁺, ¹H NMR (400 MHZ, MeOH-d₄) δ 9.04 (s, 1H), 8.16 (s, 1H), 7.96 (d, 1H), 7.35-7.28 (m, 3H), 7.22 (qd, 3H), 5.10 (q, 1H), 4.40 (q, 2H), 4.29 (qt. 2H), 1.64 (t, 3H), 1.52 (t, 3H), 1.17 (d, 3H).

Further elution provided Peak 2, 10 mg as a light yellow solid, Example 108, (R)-1-(7-ethoxy-4-(1-ethyl-3-phenyl-1H-pyrazol-4-yl)quinazolin-6-yl)ethan-1-ol or (S)-1-(7-ethoxy-4-(1-ethyl-3-phenyl-1H-pyrazol-4-yl)quinazolin-6-yl)ethan-1-ol. ¹H NMR (400 MHz, MeOH-d₄) δ 9.04 (s, 1H), 8.16 (s, 1H), 7.96 (d, 1H), 7.35-7.28 (m, 3H), 7.22 (qd, 3H), 5.10 (q, 1H), 4.40 (q, 2H), 4.29 (qt, 2H), 1.64 (t, 3H), 1.52 (t, 3H), 1.17 (d, 3H).

### Example 109 and 110: (R)-1-(7-ethoxy-4-(1-isopropyl-3-phenyl-1H-pyrazol-4-yl)quinazolin-6-yl)ethan-1-ol and (S)-1-(7-ethoxy-4-(1-isopropyl-3-phenyl-1H-pyrazol-4-yl)quinazolin-6-yl)ethan-1-ol

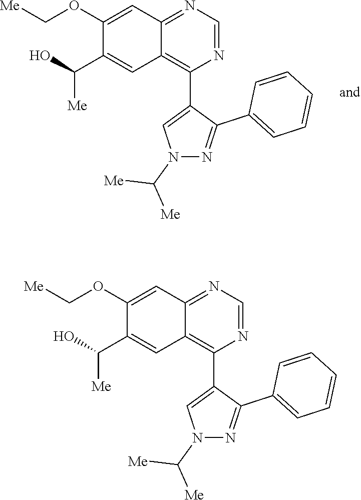 [SMILES: CCOc1cc2ncnc(-c3cn(C(C)C)nc3-c3ccccc3)c2cc1[C@@H](C)O ; CCOc1cc2ncnc(-c3cn(C(C)C)nc3-c3ccccc3)c2cc1[C@H](C)O]

1-(7-Ethoxy-4-(1-isopropyl-3-phenyl-1H-pyrazol-4-yl)quinazolin-6-yl)ethan-1-ol was obtained from 1-(7-ethoxy-4-(1-isopropyl-3-phenyl-1H-pyrazol-4-yl)quinazolin-6-yl)ethan-1-one (Preparation 180), following the procedure described in Examples 107 and 108. The racemic compound was further purified by prep-HPLC Column: Lux 5 um Cellulose-2, 2.12*25 cm, 5 um; Mobile Phase A: Hex (0.5% 2M NH₃—MeOH), Mobile Phase B: EtOH: Flow rate: 20 mL/min; Gradient: 10% B isocratic to give

Peak 1, 6.7 mg as a white solid, Example 109, (R)-1-(7-ethoxy-4-(1-isopropyl-3-phenyl-1H-pyrazol-4-yl)quinazolin-6-yl)ethan-1-ol or (S)-1-(7-ethoxy-4-(1-isopropyl-3-phenyl-1H-pyrazol-4-yl)quinazolin-6-yl)ethan-1-ol. LCMS: m/z=403 [M+H]⁺, ¹H NMR (400 MHZ, DMSO-d₆): δ=9.08 (s, 1H), 8.28 (s, 1H), 7.90 (s, 1H), 7.32-7.17 (m, 6H), 4.94 (q, 1H), 4.69 (p, 1H), 4.32-4.19 (m, 2H), 1.57 (d, 6H), 1.41 (t, 3H), 1.06 (d, 3H).

Further elution provided Peak 2, 5.4 mg, as a white solid, Example 110, (S)-1-(7-ethoxy-4-(1-isopropyl-3-phenyl-1H-pyrazol-4-yl)quinazolin-6-yl)ethan-1-ol or (R)-1-(7-ethoxy-4-(1-isopropyl-3-phenyl-1H-pyrazol-4-yl)quinazolin-6-yl)ethan-1-ol. LCMS: m/z=403 [M+H]⁺; ¹H NMR (400 MHZ, DMSO-d₆) δ=9.08 (s, 1H), 8.29 (s, 1H), 7.90 (s, 1H), 7.33-7.18 (m, 6H), 5.06 (d, 1H), 4.94 (s, 1H), 4.74-4.64 (m, 1H), 4.30-4.20 (m, 2H), 1.57 (d, 6H), 1.41 (t, 3H), 1.05 (d, 3H).

### Example 111 and 112: (R)-1-(4-(1-cyclopropyl-3-phenyl-1H-pyrazol-4-yl)-7-ethoxyquinazolin-6-yl)ethan-1-ol and (S)-1-(4-(1-cyclopropyl-3-phenyl-1H-pyrazol-4-yl)-7-ethoxyquinazolin-6-yl)ethan-1-ol

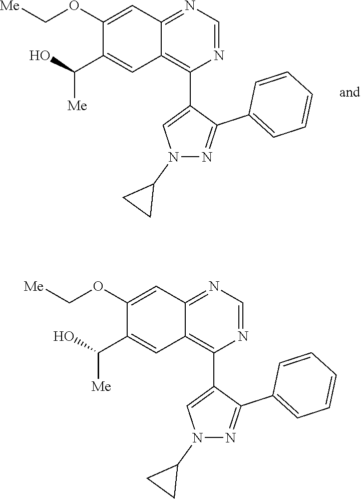 [SMILES: CCOc1cc2ncnc(-c3cn(C4CC4)nc3-c3ccccc3)c2cc1[C@@H](C)O ; CCOc1cc2ncnc(-c3cn(C4CC4)nc3-c3ccccc3)c2cc1[C@H](C)O]

1-(4-(1-Cyclopropyl-3-phenyl-1H-pyrazol-4-yl)-7-ethoxyquinazolin-6-yl)ethan-1-ol was obtained from 1-(4-(1-cyclopropyl-3-phenyl-1H-pyrazol-4-yl)-7-ethoxyquinazolin-6-yl)ethan-1-one (Preparation 176), following the procedure described in Examples 107 and 108. The racemate was further purified by prep-Chiral-HPLC, Column: CHIRALPAK AD-H, 5*25 cm, 5 μm; Mobile Phase A: Hex (0.5% 2M NH₃—MeOH), Mobile Phase B: EtOH: Flow rate: 20 mL/min; Gradient: 15% B isocratic to give

Peak 1, 15.3 mg as a white solid, Example 111, (R)-1-(4-(1-cyclopropyl-3-phenyl-1H-pyrazol-4-yl)-7-ethoxyquinazolin-6-yl)ethan-1-ol or (S)-1-(4-(1-cyclopropyl-3-phenyl-1H-pyrazol-4-yl)-7-ethoxyquinazolin-6-yl)ethan-1-ol, LCMS m/z=401 [M+H]⁺ 1H NMR (400 MHZ, MeOH-d₄) δ 9.01 (s, 1H), 8.16 (s, 1H), 7.91 (d, 1H), 7.31-7.22 (m, 3H), 7.25-7.13 (m, 3H), 5.17-5.03 (m, 1H), 4.26 (qd, 2H), 3.86 (tt, 1H), 1.49 (t, 3H), 1.35-1.24 (m, 2H), 1.27-1.09 (m, 5H).

Further elution provided Peak 2, 8.6 mg, as a white solid, Example 112, (S)-1-(4-(1-cyclopropyl-3-phenyl-1H-pyrazol-4-yl)-7-ethoxyquinazolin-6-yl)ethan-1-ol or (R)-1-(4-(1-cyclopropyl-3-phenyl-1H-pyrazol-4-yl)-7-ethoxyquinazolin-6-yl)ethan-1-ol. LCMS: m/z=401 [M+H]⁺, 1NMR (400 MHz, MeOH-d₄) δ 9.01 (s, 1H), 8.16 (s, 1H), 7.91 (d, 1H), 7.31-7.22 (m, 3H), 7.25-7.13 (m, 3H), 5.17-5.03 (m, 1H), 4.26 (qd, 2H), 3.86 (tt, 1H), 1.49 (t, 3H), 1.35-1.24 (m, 2H), 1.27-1.09 (m, 5H)

### Example 113 and 114: (S)-1-(7-isopropoxy-4-(1-methyl-3-phenyl-1H-pyrazol-4-yl)quinazolin-6-yl)ethan-1-ol and (R)-1-(7-isopropoxy-4-(1-methyl-3-phenyl-1H-pyrazol-4-yl)quinazolin-6-yl)ethan-1-ol

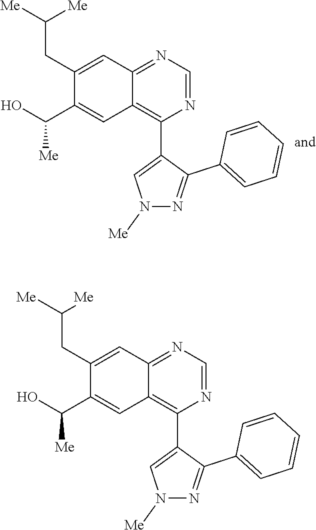 [SMILES: CC(C)Cc1cc2ncnc(-c3cn(C)nc3-c3ccccc3)c2cc1[C@H](C)O ; CC(C)Cc1cc2ncnc(-c3cn(C)nc3-c3ccccc3)c2cc1[C@@H](C)O]

NaBH₄ (61.9 mg, 1.63 mmol) was added to 1-(7-isopropoxy-4-(1-methyl-3-phenyl-1H-pyrazol-4-yl)quinazolin-6-yl)ethan-1-one (Preparation 174, 210 mg, 0.543 mmol) in MeOH (5 mL) and the reaction was stirred at 25° C. for 2 h. The reaction was quenched by the addition of aq. NH—Cl and extracted with EtOAc. The combined organic extracts were concentrated in vacuum and the crude was purified by Prep-HPLC: Column: CHIRALPAK IA, 2*25 cm, 5 μm; Mobile Phase A: Hex (0.5% 2M NH₃—MeOH), Mobile Phase B: EtOH: Flow rate: 20 mL/min; Gradient: 10% B isocratic, to afford 1-(7-isopropoxy-4-(1-methyl-3-phenyl-1H-pyrazol-4-yl)quinazolin-6-yl)ethan-1-ol (110 mg, yield 52.3%) as a light yellow solid. This was further purified by Column: CHIRALPAK IA, 2*25 cm, 5 μm; Mobile Phase A: Hex (0.5% 2M NH₃—MeOH), Mobile Phase B: EtOH; Flow rate: 20 mL/min; Gradient: 10% B isocratic, to afford

Peak 1, 31.2 mg, yield: 62.7% as a light yellow solid, Example 113, (S)-1-(7-isopropoxy-4-(1-methyl-3-phenyl-1H-pyrazol-4-yl)quinazolin-6-yl)ethan-1-ol or (R)-1-(7-isopropoxy-4-(1-methyl-3-phenyl-1H-pyrazol-4-yl)quinazolin-6-yl)ethan-1-ol. LCMS: m/z=389 [M+H]⁺; 1H NMR (300 MHZ, DMSO-d₆) δ 9.06 (s, 1H), 8.19 (s, 1H), 7.93 (s, 1H), 7.32 (s, 1H), 7.26 (qd, 2H), 7.21 (q, 3H), 5.05 (s, 1H), 4.93 (p, 2H), 4.04 (s, 3H), 1.36 (dd, 6H), 1.24 (s, 1H), 1.09 (d, 3H)

Further elution provided Peak 2, 24.9 mg, as a light yellow solid, Example 114. (R)-1-(7-isopropoxy-4-(1-methyl-3-phenyl-1H-pyrazol-4-yl)quinazolin-6-yl)ethan-1-ol or (S)-1-(7-isopropoxy-4-(1-methyl-3-phenyl-1H-pyrazol-4-yl)quinazolin-6-yl)ethan-1-ol. LCMS: m/z=389 [M+H]⁺; 1H NMR (300 MHz, DMSO-d₆) δ 9.06 (s, 1H), 8.19 (s, 1H), 7.93 (s, 1H), 7.32 (s, 1H), 7.30-7.20 (m, 2H), 7.21 (q, 3H), 5.05 (d, 1H), 4.94 (dt, 1H), 4.92 (s, 1H), 4.04 (s, 3H), 1.36 (dd, 6H), 1.09 (d, 3H).

### Example 115 and 116: (R)-1-(7-methoxy-4-(1-methyl-3-phenyl-1H-pyrazol-4-yl)quinazolin-6-yl)ethan-1-ol and (S)-1-(7-methoxy-4-(1-methyl-3-phenyl-1H-pyrazol-4-yl)quinazolin-6-yl)ethan-1-ol

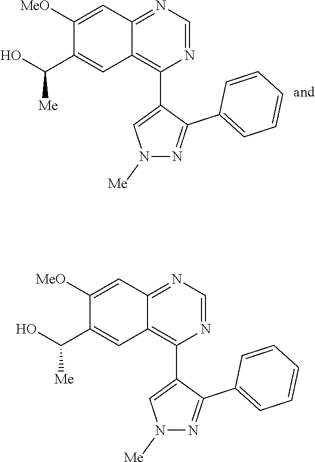 [SMILES: COc1cc2ncnc(-c3cn(C)nc3-c3ccccc3)c2cc1[C@@H](C)O ; COc1cc2ncnc(-c3cn(C)nc3-c3ccccc3)c2cc1[C@H](C)O]

1-(7-Methoxy-4-(1-methyl-3-phenyl-1H-pyrazol-4-yl)quinazolin-6-yl)ethan-1-ol (Example 100, 70 mg, 0.194 mmol) was purified by Prep-HPLC Column: CHIRAL ART Cellulose-SB, 3*25 cm, 5 μm; Mobile Phase A: Hex:DCM=3:1 (0.5% 2M NH₃—MeOH), Mobile Phase B: IPA Flow rate: 20 mL/min; Gradient: 10% B isocratic to give

Peak 1, 27.2 mg as a white solid, Example 115, (R)-1-(7-methoxy-4-(1-methyl-3-phenyl-1H-pyrazol-4-yl)quinazolin-6-yl)ethan-1-ol or (S)-1-(7-methoxy-4-(1-methyl-3-phenyl-1H-pyrazol-4-yl)quinazolin-6-yl)ethan-1-ol. LCMS: m/z=361 [M+H]⁺. 1H NMR (300 MHZ, DMSO-d₆) δ 9.09 (s, 1H), 8.21 (s, 1H), 7.90 (s, 1H), 7.34 (s, 1H), 7.28-7.12 (m, 5H), 5.09 (s, 1H), 4.94 (q, 1H), 4.01 (d, 6H), 1.06 (d, 3H).

Further elution provided Peak 2, 28.9 mg, as a white solid, Example 116, (S)-1-(7-methoxy-4-(1-methyl-3-phenyl-1H-pyrazol-4-yl)quinazolin-6-yl)ethan-1-ol or (R)-1-(7-methoxy-4-(1-methyl-3-phenyl-1H-pyrazol-4-yl)quinazolin-6-yl)ethan-1-ol. LCMS: m/z=361 [M+H]⁺. 1H NMR (300 MHz, DMSO-d₆) δ 9.10 (s, 1H), 8.22 (s, 1H), 7.90 (s, 1H), 7.34 (s, 1H), 7.28-7.13 (m, 5H), 4.94 (q, 1H), 4.04 (s, 3H), 3.98 (s, 3H), 1.06 (d, 3H).

### Example 117 and 118: (S)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol and (R)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol

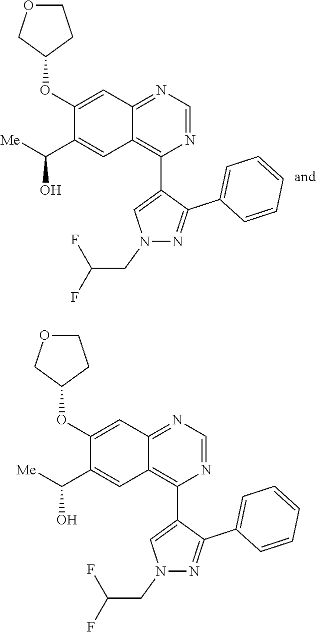

To a solution of(S)-1-(4-(1-(2,2-difluoroethyl)-3-phenyl-1H-pyrazol-4-yl)-7-((tetrahydrofuran-3-yl)oxy) quinazolin-6-yl)ethan-1-one (Preparation 175, 50 mg, 0.107 mmol), in MeOH (10 mL) was added NaBH₄ (8.09 mg, 0.214 mmol) and the reaction was stirred at rt for 1 h. The reaction was quenched with water and the mixture concentrated to dryness. The residue was purified on prep-TLC with DCM:MeOH=30:1. The product was further purified by Chiral-HPLC, Column: CHIRALPAK ID, 2*25 cm, 5 um; Mobile Phase A: Hex:DCM=3:1 (0.5% 2M NH₃—MeOH), Mobile Phase B: EtOH; Flow rate: 20 mL/min; Gradient: 5% B isocratic to afford

Peak 1, 19.5 mg, as a white solid, Example 117, (S)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol or (R)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol. LCMS: m/z=467 [M+H]⁺1H NMR (400 MHZ, DMSO-d₆) δ 9.11 (s, 1H), 8.31 (s, 1H), 7.91 (s, 1H), 7.40-7.22 (m, 3H), 7.25-7.18 (m, 3H), 6.55 (t, 1H), 5.39-5.31 (m, 1H), 5.06 (s, 1H), 4.86 (m, 3H), 3.99 (dd, 1H), 3.90-3.77 (m, 3H), 2.40-2.26 (m, 1H), 2.05 (m, 1H), 1.07 (d, 3H).

Further elution provided Peak 2, 8.6 mg, as a white solid, Example 118, (R)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol or (S)-1-(4-(1-(2,2-difluoroethyl)-3-phenyl-1H-pyrazol-4-yl)-7-(((S)-tetrahydrofuran-3-yl)oxy) quinazolin-6-yl)ethan-1-ol. LCMS: m/z=467 [M+H]⁺. 1H NMR (400 MHZ, DMSO-d₆) δ 9.11 (s, 1H), 8.31 (s, 1H), 7.91 (s, 1H), 7.40-7.22 (m, 3H), 7.25-7.18 (m, 3H), 6.55 (t, 1H), 5.39-5.31 (m, 1H), 5.06 (s, 1H), 4.86 (m, 3H), 3.99 (dd, 1H), 3.90-3.77 (m, 3H), 2.40-2.26 (m, 1H), 2.05 (m, 1H), 1.07 (d, 3H).

### Example 119: (7-methoxy-4-(1-methyl-3-phenyl-1H-pyrazol-4-yl)quinazolin-6-yl)methanol

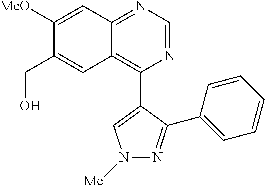 [SMILES: COc1cc2ncnc(-c3cn(C)nc3-c3ccccc3)c2cc1CO]

A solution of ethyl 7-methoxy-4-(1-methyl-3-phenyl-1H-pyrazol-4-yl)quinazoline-6-carboxylate (Preparation 181, 50 mg, 0.129 mmol) in anhydrous THF (3 mL) was cooled to −30° C. and DIBAL-H in toluene (1.0 M, 0.46 mL) was added dropwise over 5 min under an atmosphere of N₂. The reaction was stirred at −10° C. for 1 h then quenched with HCl and partitioned between EtOAc and H₂O. The aqueous phase was re-extracted with EtOAc (3×) and the combined organic fractions were washed with brine, dried over Na₂SO₄, filtered and concentrated in vacuo resulting in a crude yellow oil. The residue was purified by prep-HPLC (Column: YMC-Actus Triart C18, 30 mm×150 mm, 5 um; Mobile Phase A: Water (10 mmol/L NH₄HCO₃+0.1% NH₃·H₂O), Mobile Phase B: MeCN; Flow rate: 60 mL/min; Gradient: 26% B to 36% B in 8 min, to give the title compound (14.3 mg, yield: 32.1%) as a white solid. LCMS m/z=347 [M+H]⁺1H NMR (300 MHz, DMSO-d₆) δ 9.08 (s, 1H), 8.20 (s, 1H), 7.94 (d, 1H), 7.37 (s, 1H), 7.34-7.28 (m, 2H), 7.27-7.14 (m, 3H), 4.53 (d, 2H), 4.03 (s, 3H), 3.99 (s, 3H).

### Example 120:7-methoxy-6-(methoxymethyl)-4-(1-methyl-3-phenyl-1H-pyrazol-4-yl)quinazoline

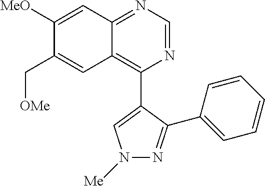 [SMILES: COCc1cc2c(-c3cn(C)nc3-c3ccccc3)ncnc2cc1OC]

To a mixture of (7-methoxy-4-(1-methyl-3-phenyl-1H-pyrazol-4-yl)quinazolin-6-yl)methanol (Example 119, 50 mg, 0.144 mmol) in DMF (4 mL) was added NaH (10.3 mg, 3.33 mmol) at 0° C., the mixture stirred at 0° C. for 15 min, then iodomethane (61.4 mg, 0.433 mmol) was added and the reaction mixture stirred at 25° C. for 3 h. The reaction was added to ice water then concentrated under vacuum. The residue was purified by prep-HPLC (Column: XBridge Prep OBD C18 Column, 30×150 mm 5 um; Mobile Phase A: Water (10 mmol/L NH₄HCO₃+0.1% NH₃·H₂O), Mobile Phase B: MeCN: Flow rate: 60 mL/min; Gradient: 23% B to 53% B in 8 min, to give the title compound (16.9 mg, yield: 32.4%) as a yellow solid. LCMS m/z=361 [M+H]⁺1H NMR (300 MHz, DMSO-d₆) δ 9.13 (s, 1H), 8.21 (s, 1H), 7.73 (d, 1H), 7.39 (s, 1H), 7.31-7.12 (m, 5H), 4.38 (d, 2H), 4.01 (d, 6H), 3.13 (s, 3H).

### Example 121:2-(7-methoxy-4-(1-methyl-3-phenyl-1H-pyrazol-4-yl)quinazolin-6-yl) propan-2-ol

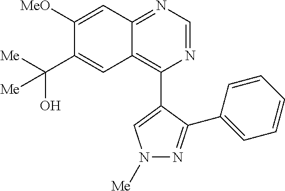 [SMILES: COc1cc2ncnc(-c3cn(C)nc3-c3ccccc3)c2cc1C(C)(C)O]

A solution of ethyl 7-methoxy-4-(1-methyl-3-phenyl-1H-pyrazol-4-yl)quinazoline-6-carboxylate (Preparation 181, 100 mg, 0.257 mmol) in anhydrous THF (5 mL) was cooled to −10° C. and MeMgBr (3.0 M in THF, 0.25 mL) was added over 5 min under an atmosphere of Ar. The reaction was stirred at −10° C. for 1 h, then quenched with sat. NH₄Cl (2.0 mL) and partitioned between EtOAc and H₂O. The aqueous was extracted with EtOAc (3×) and the combined organic fractions were washed with brine, dried over Na₂SO₄, filtered and concentrated in vacuo resulting in a crude yellow oil. The residue was purified by prep-HPLC (Column YMC-Actus Triart C18, 30 mm×150 mm, 5 um; Mobile Phase A: Water (10 mmol/L NH₄HCO₃+0.1% NH₃·H₂O), Mobile Phase B: MeCN: Flow rate: 60 mL/min; Gradient: 25% B to 55% B in 8 min: to give the title compound (49.6 mg, yield: 51.5%) as a white solid. LCMS m/z=375 [M+H]⁺ 1H NMR (300 MHz, DMSO-d₆) δ 9.06 (s, 1H), 8.24 (s, 1H), 8.16 (s, 1H), 7.36 (s, 1H), 7.31-7.24 (m, 2H), 7.21 (dp, 3H), 5.04 (s, 1H), 4.03 (s, 3H), 4.00 (s, 3H), 1.41 (s, 6H).

### Example 122:2-(4-(1-(2,2-difluoroethyl)-3-phenyl-1H-pyrazol-4-yl)-7-methoxyquinazolin-6-yl) propan-2-ol

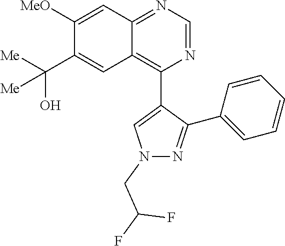 [SMILES: COc1cc2ncnc(-c3cn(CC(F)F)nc3-c3ccccc3)c2cc1C(C)(C)O]

The title compound was obtained as a white solid, 9.9 mg, 20%, from ethyl 4-(1-(2,2-difluoroethyl)-3-phenyl-1H-pyrazol-4-yl)-7-methoxyquinazoline-6-carboxylate (Preparation 182), following a similar procedure to that described in Example 121, except the crude was purified by HPLC: Column: XSelect CSH Prep C18 OBD Column, 19*250 mm, 5 um; Mobile Phase A: Water (10 mmol/L NH₄HCO₃+0.1% NH₃·H₂O), Mobile Phase B: MeCN; Flow rate: 20 mL/min; Gradient: 40% B to 42% B in 10 min. LCMS: m/z=425 [M+H]⁺; ¹H NMR (300 MHz, DMSO-d₆) δ 9.08 (s, 1H), 8.34 (s, 1H), 8.14 (s, 1H), 7.37 (s, 1H), 7.33-7.17 (m, 5H), 6.55 (t, 1H), 4.99 (s, 1H), 4.84 (td, 2H), 4.00 (s, 3H), 1.41 (s, 6H).

### Example 123: (4-(1-(2,2-difluoroethyl)-3-phenyl-1H-pyrazol-4-yl)-7-ethoxyquinazolin-6-yl)methanol

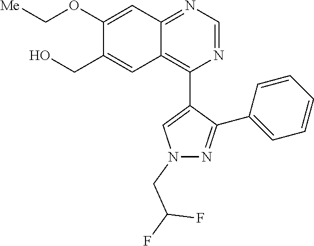 [SMILES: CCOc1cc2ncnc(-c3cn(CC(F)F)nc3-c3ccccc3)c2cc1CO]

Pd(PPh₃)₂Cl₂ (50.3 mg, 0.072 mmol) was added to 6-bromo-4-(1-(2,2-difluoroethyl)-3-phenyl-1H-pyrazol-4-yl)-7-ethoxyquinazoline (Preparation 159, 330 mg, 0.72 mmol) and (tributylstannyl)methanol (343 mg, 1.07 mmol) in dioxane (10 mL) and the reaction was heated at 80° C. for 16 h under N₂. The reaction mixture was diluted with EtOAc (100 mL) and washed sequentially with water (100 mL×3) and saturated brine (100 mL). The organic layer was dried over Na₂SO₄, filtered and evaporated under reduced pressure. The crude product was purified by prep-TLC with DCM:MeOH=25:1 and further purified by Prep-HPLC Column: YMC-Actus Triart C18, 30 mm×150 mm, 5 um; Mobile Phase A: Water (10 mmol/L NH₂HCO₃+0.1% NH₃·H₂O), Mobile Phase B: MeCN: Flow rate: 60 mL/min; Gradient: 30% B to 65% B in 8 min; to give the title compound, 39.5 mg (13.4%) as a white solid. LCMS: m/z=411 [M+H]⁺, 1H NMR (400 MHZ, DMSO-d₆) δ 9.08 (s, 1H), 8.29 (s, 1H), 7.94-7.89 (m, 1H), 7.41-7.26 (m, 3H), 7.29-7.20 (m, 3H), 6.55 (t, 0H), 5.21 (t, 1H), 4.83 (td, 2H), 4.52 (dd, 2H), 4.27 (q, 2H), 1.41 (t, 3H).

### Example 124: (7-ethoxy-4-(1-ethyl-3-phenyl-1H-pyrazol-4-yl)quinazolin-6-yl)methanol

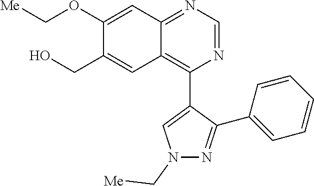 [SMILES: CCOc1cc2ncnc(-c3cn(CC)nc3-c3ccccc3)c2cc1CO]

The title compound was obtained as a white solid, 19.9 mg, 22.5% yield from 6-bromo-7-ethoxy-4-(1-ethyl-3-phenyl-1H-pyrazol-4-yl)quinazoline (Preparation 93), following a similar procedure to that described in Example 123. LCMS m/z=375 [M+H]⁺, 1H NMR (400 MHZ, DMSO-d₆) δ 9.06 (s, 1H), 8.24 (s, 1H), 7.94 (d, 1H), 7.36-7.28 (m, 3H), 7.21 (p, 3H), 5.20 (t, 1H), 4.53 (dd, 2H), 4.29 (dq, 4H), 1.52 (t, 3H), 1.42 (t, 3H).

### Example 125: (7-ethoxy-4-(1-isopropyl-3-phenyl-1H-pyrazol-4-yl)quinazolin-6-yl)methanol

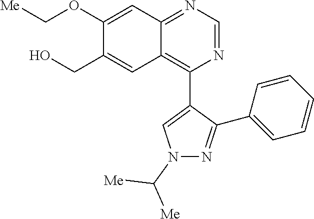 [SMILES: CCOc1cc2ncnc(-c3cn(C(C)C)nc3-c3ccccc3)c2cc1CO]

The title compound was obtained as a white solid, 5.8 mg, from 6-bromo-7-ethoxy-4-(1-isopropyl-3-phenyl-1H-pyrazol-4-yl)quinazoline (Preparation 161) and (tributylstannyl)methanol following a similar procedure to that described in Example 123, except the compound was purified by prep-HPLC, Column: XBridge Prep Phenyl OBD Column, 19*150 mm, 5 um; Mobile Phase A: Water (10 mmol/L NH₄HCO₃+0.1% NH₃·H₂O), Mobile Phase B: MeOH; Flow rate: 25 mL/min; Gradient: 55% B to 73% B in 7 min. LCMS: m/z=250 [M+H]⁺, ¹H NMR (400 MHZ, DMSO-d₆) δ 9.06 (s, 1H), 8.26 (s, 1H), 7.94 (d, 1H), 7.35-7.27 (m, 3H), 7.22 (dt, 3H), 5.19 (s, 1H), 4.67 (h, 1H), 4.52 (d, 2H), 4.27 (q, 2H), 1.57 (d, 6H), 1.42 (t, 3H).

### Example 126:1-(4-(1-(2,2-difluoroethyl)-3-phenyl-1H-pyrazol-4-yl)-7-methoxyquinazolin-6-yl)cyclopropan-1-ol

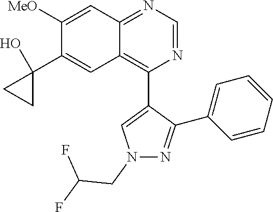 [SMILES: COc1cc2ncnc(-c3cn(CC(F)F)nc3-c3ccccc3)c2cc1C1(O)CC1]

NaBO₃ (2.07 g, 13.5 mmol) was added to 4-(1-(2,2-difluoroethyl)-3-phenyl-1H-pyrazol-4-yl)-7-methoxy-6-(1-(4,4,5,5-tetramethyl-1,3,2-dioxaborolan-2-yl)cyclopropyl)quinazoline (Preparation 183, 2.4 g, 4.50 mmol) in THF/H₂O (20 mL/20 mL) and the reaction was stirred at rt for 2 h. The mixture was diluted with DCM (200 mL), washed with brine (200 mL×2), the organic layer was dried over Na₂SO₄ and concentrated under vacuum. The residue was purified by prep-HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 21% B to 54% B in 7 min, to give the title compound, 689.1 mg as a white solid. LCMS: m/z=423 [M+H]⁺: 1H NMR (400 MHZ, DMSO-d₆): ppm δ=9.12 (s, 1H), 8.35 (s, 1H), 7.67 (s, 1H), 7.35 (s, 1H), 7.33-7.25 (m, 2H), 7.29-7.17 (m, 3H), 6.56 (tt, 1H), 5.54 (s, 1H), 4.84 (td, 2H), 3.99 (s, 3H), 0.87-0.75 (m, 2H), 0.51-0.43 (m, 2H).

### Example 127:4-(4-(1-(2,2-difluoroethyl)-3-phenyl-1H-pyrazol-4-yl)-7-methoxyquinazolin-6-yl)tetrahydro-2H-pyran-4-ol

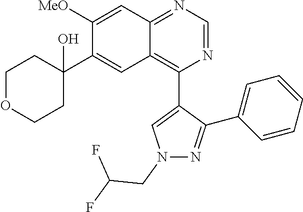 [SMILES: COc1cc2ncnc(-c3cn(CC(F)F)nc3-c3ccccc3)c2cc1C1(O)CCOCC1]

n-Butyllithium (0.1 mL, 2.5 M, 0.25 mmol) was added dropwise to 6-bromo-4-(1-(2,2-difluoroethyl)-3-phenyl-1H-pyrazol-4-yl)-7-methoxyquinazoline (Preparation 157, 44.5 mg, 0.1 mmol) and tetrahydro-4H-pyran-4-one (40 mg, 0.4 mmol) in THF (10 mL) at −78° C. under N₂, and the reaction was warmed to rt slowly and stirred until the starting material had been consumed. The mixture was concentrated in vacuo and the residue was purified by prep-HPLC (Column: YMC-Actus Triart C18, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 23% B to 43% B in 10 min, to give the title compound, as a white solid 12.2 mg, 26%. LCMS m/z=467 [M+H]⁺. 1H NMR (300 MHz, DMSO-d₆) δ 9.09 (s, 1H), 8.37 (s, 1H), 8.19 (s, 1H), 7.41 (s, 1H), 7.30 (dd, 2H), 7.28-7.19 (m, 3H), 6.80-6.26 (m, 2H), 5.01 (s, 1H), 4.84 (td, 2H), 4.03 (s, 3H), 3.81-3.61 (m, 4H), 2.41 (dd, 2H), 1.22 (d, 2H).

### Example 128: cyclopropyl (7-methoxy-4-(1-methyl-3-phenyl-1H-pyrazol-4-yl)quinazolin-6-yl)methanol

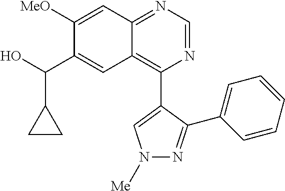 [SMILES: COc1cc2ncnc(-c3cn(C)nc3-c3ccccc3)c2cc1C(O)C1CC1]

n-Butyllithium (2.5 M, 111 μl, 0.278 mmol) was added dropwise to a solution of 6-bromo-7-methoxy-4-(1-methyl-3-phenyl-1H-pyrazol-4-yl)quinazoline (Preparation 91, 100 mg, 0.253 mmol) in DCM (3 mL) and the solution stirred for 20 mins. A solution of cyclopropanecarboxaldehyde (21.3 mg, 0.3 mmol) in DCM (1 mL) was added dropwise and the reaction allowed to warm to rt. The reaction was quenched with sat NH₄Cl solution and the mixture extracted with DCM. The combined organic layer was dried and concentrated in vacuo. The crude material was purified by prep-HPLC (Column: XBridge Shield RP18 OBD, 30*150 mm, 5 um; mobile phase: 0-40% (0.1% TFA modified water/MeCN) at 60 mL/min, to give the title compound, 9.2 mg, 9.41% yield. 1H NMR (MeOH-d₄) δ: 9.12 (d, 1H), 8.19 (d, 1H), 8.04 (s, 1H), 7.42 (s, 1H), 7.35 (dd, 2H), 7.23 (t, 3H), 4.46 (d, 1H), 4.12 (d, 3H), 4.07 (d, 3H), 0.71 (p, 1H), 0.40 (t, 1H), 0.27 (p, 3H).

### Example 129 and 130: (S)-2-(7-methoxy-4-(1-methyl-3-phenyl-1H-pyrazol-4-yl)quinazolin-6-yl)-4-methylmorpholine and (R)-2-(7-methoxy-4-(1-methyl-3-phenyl-1H-pyrazol-4-yl)quinazolin-6-yl)-4-methylmorpholine

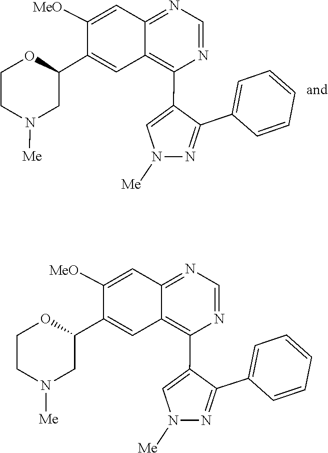 [SMILES: COc1cc2ncnc(-c3cn(C)nc3-c3ccccc3)c2cc1[C@H]1CN(C)CCO1 ; COc1cc2ncnc(-c3cn(C)nc3-c3ccccc3)c2cc1[C@@H]1CN(C)CCO1]

6-(7-Methoxy-4-(1-methyl-3-phenyl-1H-pyrazol-4-yl)quinazolin-6-yl)-3,4-dihydro-2H-1,4-oxazine (Preparation 185, 40 mg, 0.10 mmol) and bis(formaldehyde)(0.5 mL) were added to MeOH (3 mL) and AcOH (0.3 mL) and the solution stirred at rt for 30 min. NaCNBH₃ (12.5 mg, 0.20 mmol) was added and the reaction stirred at rt for 2 h. The reaction mixture was extracted with EtOAc (3×30 mL), the combined organic layers were dried over Na₂SO₄ and concentrated under vacuum. The crude product was purified by Prep-HPLC Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN: Flow rate: 60 mL/min; Gradient: 11% to 41% B in 10 min, to give 7-methoxy-4-(1-methyl-3-phenyl-1H-pyrazol-4-yl)-6-(4-methylmorpholin-2-yl)quinazoline, 10 mg, 24% as a white solid. LCMS: m/z=416 [M+H]⁺. This compound (10 mg, 0.024 mmol) was further purified by Prep-Chiral-HPLC: CHIRALPAK IH, 2*25 cm, 5 μm; Mobile Phase A: Hex (0.5% 2M NH₃—MeOH), Mobile Phase B: EtOH: Flow rate: 20 mL/min; Gradient: 30% B isocratic, to give

Peak 1, 2.5 mg, 25% as a white solid, Example 129, (S)-2-(7-methoxy-4-(1-methyl-3-phenyl-1H-pyrazol-4-yl)quinazolin-6-yl)-4-methylmorpholine or (R)-2-(7-methoxy-4-(1-methyl-3-phenyl-1H-pyrazol-4-yl)quinazolin-6-yl)-4-methylmorpholine. LCMS: m/z=416 [M+H]⁺, 1H NMR (400 MHz, DMSO-d₆) δ 9.16 (s, 1H), 8.24 (s, 1H), 7.66 (d, 1H), 7.34 (s, 1H), 7.20 (s, 5H), 4.67-4.56 (m, 1H), 4.04 (s, 3H), 3.97 (s, 3H), 3.90-3.81 (m, 1H), 3.55 (t, 1H), 2.59 (t, 2H), 2.13 (s, 3H), 1.97 (dt, 1H), 0.97 (t, 1H).

Further elution provided Peak 2 as a white solid, 3.6 mg, 36%, Example 130. LCMS: m/z=416 [M+H]⁺, 1H NMR (400 MHz, DMSO-d₆) δ 9.16 (s, 1H), 8.24 (s, 1H), 7.66 (d, 1H), 7.34 (s, 1H), 7.20 (s, 5H), 4.67-4.57 (m, 1H), 4.04 (s, 3H), 3.97 (s, 3H), 3.90-3.81 (m, 1H), 3.63-3.49 (m, 1H), 2.59 (t, 2H), 2.11 (s, 3H), 1.93 (td, 1H), 1.04-0.93 (m, 1H).

### Example 131:7-methoxy-6-(1-methyl-2,5-dihydro-1H-pyrrol-3-yl)-4-(1-methyl-3-phenyl-1H-pyrazol-4-yl)quinazoline

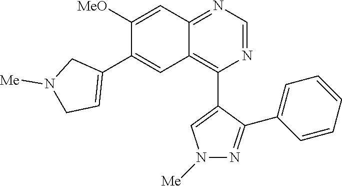 [SMILES: COc1cc2ncnc(-c3cn(C)nc3-c3ccccc3)c2cc1C1=CCN(C)C1]

Paraformaldehyde (0.053 g, 0.710 mmol) and MeOH (3 mL) were added to 6-(2,5-dihydro-1H-pyrrol-3-yl)-7-methoxy-4-(1-methyl-3-phenyl-1H-pyrazol-4-yl)quinazoline trifluoroacetate (Preparation 186, 54 mg, 0.142 mmol) and the solution stirred at rt. The solution was concentrated in vacuo and the process repeated. MeOH (3 mL) and Na(OAc)₃BH (0.150 g, 0.71 mmol) were added to the residue and the reaction was stirred at rt. The reaction mixture was concentrated in vacuo and the residue purified by prep-HPLC to afford the title compound. 1H NMR (MeOH-d₄) δ: 9.17 (s, 1H), 8.20 (d, 1H), 7.62 (s, 1H), 7.45 (s, 1H), 7.36 (dd, 2H), 7.33-7.23 (m, 3H), 6.17-6.13 (m, 1H), 4.45 (dd, 2H), 4.10 (d, 6H), 4.01 (d, 1H), 3.04 (s, 3H).

### Example 132 and 133: (S)-7-methoxy-4-(3-phenyl-1H-pyrazol-4-yl)-6-(tetrahydro-2H-pyran-2-yl)quinazoline and (R)-7-methoxy-4-(3-phenyl-1H-pyrazol-4-yl)-6-(tetrahydro-2H-pyran-2-yl)quinazoline

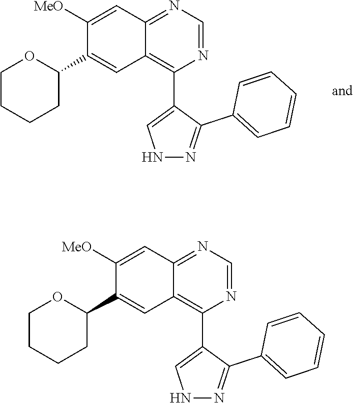 [SMILES: COc1cc2ncnc(-c3c[nH]nc3-c3ccccc3)c2cc1[C@@H]1CCCCO1 ; COc1cc2ncnc(-c3c[nH]nc3-c3ccccc3)c2cc1[C@H]1CCCCO1]

A solution of 6-bromo-7-methoxy-4-(1-methyl-3-phenyl-1H-pyrazol-4-yl)quinazoline (Preparation 91, 100 mg, 0.253 mmol) in DCM (3 mL) was cooled to −78° C. n-Butyllithium (253 μL, 0.632 mmol) was added dropwise and the solution stirred for 30 min. A solution of 5-chloropentanal (36.6 mg, 0.304 mmol) was added and the reaction warmed slowly to rt and stirred for 2 h. The mixture was partitioned between water and DCM and the organic layer concentrated in vacuo. The crude product was dissolved in DMF (1 mL), sodium hydride (25.3 mg, 0.632 mmol) was added and the solution stirred at rt for 1 h. The reaction was quenched with sat. NH₄Cl and extracted with DCM. The combined organic layer was evaporated under reduced pressure and the residue purified by HPLC Column: XBridge Shield RP18 OBD Column, 30*150 mm, 5 um; 0-40% (0.1% TFA modified water/MeCN) at 60 mL/min to give 7-methoxy-4-(1-methyl-3-phenyl-1H-pyrazol-4-yl)-6-(tetrahydro-2H-pyran-2-yl)quinazoline, 27.9 mg, 27.5% yield. 7-Methoxy-4-(1-methyl-3-phenyl-1H-pyrazol-4-yl)-6-(tetrahydro-2H-pyran-2-yl)quinazoline (19 mg) was further purified by Chiral-HPLC Column: CHIRALPAK 1H, 2*25 cm, 5 um; Mobile Phase A: Hex (0.5% 2M NH₃—MeOH), Mobile Phase B: EtOH: Flow rate: 20 mL/min; Gradient: 20% B isocratic, to give

Peak 1, Example 132, 3.0 mg as an off-white solid, (S)-7-methoxy-4-(3-phenyl-1H-pyrazol-4-yl)-6-(tetrahydro-2H-pyran-2-yl)quinazoline or (R)-7-methoxy-4-(3-phenyl-1H-pyrazol-4-yl)-6-(tetrahydro-2H-pyran-2-yl)quinazoline. LCMS: m/z=401 [M+H]⁺. 1H NMR (300 MHz, DMSO-d₆) δ 9.12 (s, 1H), 8.21 (s, 1H), 7.67 (s, 1H), 7.32 (s, 1H), 7.20 (d, 5H), 4.46 (d, 1H), 4.03 (s, 3H), 3.96 (s, 3H), 3.92 (s, 1H), 3.43 (s, 1H), 1.71-1.56 (m, 2H), 1.47 (s, 3H), 0.60 (d, 1H).

and Peak 2, Example 133, 4.6 mg, as an off-white solid, (R)-7-methoxy-4-(3-phenyl-1H-pyrazol-4-yl)-6-(tetrahydro-2H-pyran-2-yl)quinazoline or (S)-7-methoxy-4-(3-phenyl-1H-pyrazol-4-yl)-6-(tetrahydro-2H-pyran-2-yl)quinazoline. LCMS: m/z=401 [M+H]⁺. 1H NMR (300 MHZ, DMSO-d₆) δ 9.12 (s, 1H), 8.21 (s, 1H), 7.67 (s, 1H), 7.32 (s, 1H), 7.26-7.15 (m, 5H), 4.46 (d, 1H), 4.03 (s, 3H), 3.96 (s, 3H), 3.92 (s, 1H), 1.61 (dd, 2H), 1.47 (s, 3H), 0.60 (d, 1H).

### Example 134: 7-ethoxy-6-((5-methyl-1H-1,2,4-triazol-1-yl)methyl)-4-(1-methyl-3-phenyl-1H-pyrazol-4-yl)quinazoline

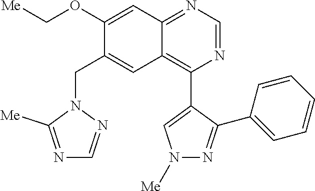 [SMILES: CCOc1cc2ncnc(-c3cn(C)nc3-c3ccccc3)c2cc1Cn1ncnc1C]

A mixture of Cs₂CO₃ (27.2 mg, 0.027 mmol), (7-ethoxy-4-(1-methyl-3-phenyl-1H-pyrazol-4-yl)quinazolin-6-yl)methyl methanesulfonate (Preparation 187, 120 mg, 0.273 mmol) and 5-methyl-1H-1,2,4-triazole (22.6 mg, 0.273 mmol) in DMF (10 mL) was stirred at 80° C. for 16 h. The reaction was extracted with EtOAc (3×20 mL), the combined organic layer was dried over Na₂SO₄ and concentrated under vacuum. The crude product was purified by Prep-HPLC Column: XBridge Prep OBD C18 Column, 30×150 mm 5 um; Mobile Phase A: Water (10 mmol/L NH₂HCO₃+0.1% NH₃·H₂O), Mobile Phase B: MeCN: Flow rate: 60 mL/min; Gradient: 21% B to 41% B in 8 min; and was further purified by: Column: CHIRAL ART Cellulose-SB, 5*25 cm, 5 um; Mobile Phase A: MtBE (0.5% 2M NH₃—MeOH), Mobile Phase B: EtOH; Flow rate: 20 mL/min; Gradient: 20% B isocratic; to give Peak 1, the title compound, 5.5 mg as a white solid, LCMS: m/z=426 [M+H]⁺, 1H NMR (400 MHZ, DMSO-d₆) δ 9.10 (s, 1H), 8.12 (s, 1H), 7.73 (s, 1H), 7.55 (s, 2H), 7.38-7.20 (m, 5H), 5.26 (s, 2H), 4.24 (dd, 2H), 4.01 (s, 3H), 2.34 (d, 3H), 1.35 (t, 3H).

### Example 135: (4-(1-cyclopropyl-3-phenyl-1H-pyrazol-4-yl)-7-ethoxyquinazolin-6-yl)methanol

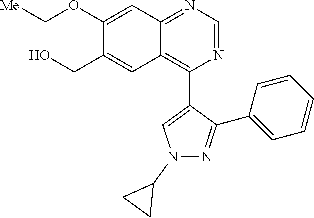 [SMILES: CCOc1cc2ncnc(-c3cn(C4CC4)nc3-c3ccccc3)c2cc1CO]

A mixture of 6-bromo-4-(1-cyclopropyl-3-phenyl-1H-pyrazol-4-yl)-7-ethoxyquinazoline (Preparation 162, 40 mg, 0.092 mmol), tributyl (1-ethoxyethenyl) stannane (58.7 mg, 0.183 mmol) and Pd(PPh₃)₂Cl₂ (15 mg, 0.021 mmol) in dioxane (20 mL) was stirred at 100° C. for 2 h. The reaction mixture was concentrated to dryness and the residue was purified on prep-TLC with DCM:MeOH=40:1. The product was further purified on prep-HPLC, Column: YMC-Actus Triart C18, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 30% B to 60% B in 8 min, to afford the title compound, (7.5 mg) as a white solid. LCMS: m/z=387 [M+H]⁺, ¹H NMR (400 MHZ, MeOH-d₄) δ 9.02 (s, 1H), 8.18 (s, 1H), 7.94 (d, 1H), 7.35-7.26 (m, 3H), 7.29-7.17 (m, 3H), 4.63 (d, 2H), 4.30 (q, 2H), 3.88 (m, 1H), 1.52 (t, 3H), 1.30 (m, 2H), 1.24-1.13 (m, 2H).

### Example 136:6-ethyl-7-methoxy-4-(1-methyl-3-phenyl-1H-pyrazol-4-yl)pyrido[3,2-d]pyrimidine

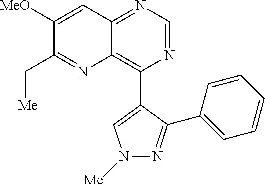 [SMILES: CCc1nc2c(-c3cn(C)nc3-c3ccccc3)ncnc2cc1OC]

A mixture of 6-chloro-7-methoxy-4-(1-methyl-3-phenyl-1H-pyrazol-4-yl)pyrido[3,2-d]pyrimidine (Preparation 242, 70 mg, 0.199 mmol), NMP (1.0 mL), THF (5.0 mL), iron (III) acetylacetonate (140.6 mg, 0.398 mmol) and ethylmagnesium bromide (79.56 mg, 0.597 mmol) under N₂ was stirred for 2 h at 0° C. in a pressure reactor. The reaction was quenched by the addition of NH₄Cl (aq), the mixture extracted with EtOAc (3×10 mL) and the combined organics concentrated in vacuo. The residue was purified by silica gel column eluting with DCM/MeOH (20/1). The product was further purified by Prep-HPLC Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 8% B to 56% B in 10 min, to give the title compound, 19.2 mg, 27.9% as an off-white solid. LCMS m/z=346 [M+H]⁺. ¹H NMR (300 MHz, DMSO-d₆) δ: 8.90 (s, 1H), 8.70 (s, 1H), 7.67 (dd, 1H), 7.31 (s, 2H), 7.04 (td, 3H), 4.02 (td, 6H), 2.88 (s, 2H), 1.10 (s, 3H).

### Example 137 to 147

The title compounds were prepared from 6-bromo-7-methoxy-4-(1-methyl-3-phenyl-1H-pyrazol-4-yl)quinazoline (Preparation 91) and the appropriate boronate using a library protocol as outlined below. Specific Procedure (1 or 2) as noted in the table.

Procedure 1: To a solution of 6-bromo-7-methoxy-4-(1-methyl-3-phenyl-1H-pyrazol-4-yl)quinazoline (Preparation 91, 71.1 mg, 0.18 mmol) and the appropriate boronate (0.27 mmol) in dioxane (3 mL) was added Cs₂CO₃ (2.0 M aq., 0.54 mmol) and Pd-132 (0.018 mmol) under N₂ and the mixture stirred at 100° C. for 16 h. The reaction mixture was diluted with water (5 mL) and extracted with EtOAc (3×4 mL). The combined organics were concentrated by speedvac and the residue purified by prep-HPLC (Column: Xtimate C18 150*25 mm*5 μm; Mobile Phase: A: NH₄OH/H₂O=0.05% v/v; B: MeCN) to give the title compounds.

Procedure 2: To a solution of 6-bromo-7-methoxy-4-(1-methyl-3-phenyl-1H-pyrazol-4-yl)quinazoline (Preparation 91, 71.1 mg, 0.18 mmol) and the appropriate boronate (0.27 mmol, 1.5 eq) in dioxane (3 mL) was added K₃PO₄ (2.0 M aq., 0.54 mmol) and Xphos Pd G3 (0.018 mmol) under N₂ and the mixture stirred at 80° C. for 16 h. The reaction mixture was washed with water (5 mL) and extracted with EtOAc (3×4 mL). The combined organics were concentrated by speedvac and the residue purified by prep-HPLC (Column: Xtimate C18 150*25 mm*5 um; Mobile Phase: A: NH₄OH/H₂O=0.05% v/v; B: MeCN) to give the title compounds.

| Example Number | Name/Structure/Procedure/Boronate/Data |
|---|---|
| 137 | 5-(7-methoxy-4-(1-methyl-3-phenyl-1H-pyrazol-4-yl)quinazolin-6-yl)-2- |
| | methylisoindolin-1-one |
| | 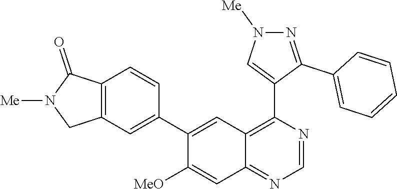 [SMILES: COc1cc2ncnc(-c3cn(C)nc3-c3ccccc3)c2cc1-c1ccc2c(c1)CN(C)C2=O] |
| | Procedure 1; Boronate: (2-methyl-1-oxoisoindolin-5-yl)boronic acid |
| | LCMS: m/z = 462 [M + H]⁺ |
| 138 | 7-methoxy-6-(2-methyl-2H-indazol-7-yl)-4-(1-methyl-3-phenyl-1H-pyrazol-4- |
| | yl)quinazoline |
| | 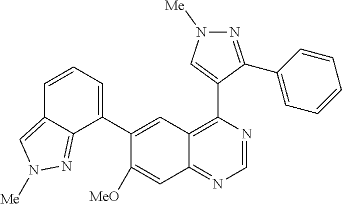 [SMILES: COc1cc2ncnc(-c3cn(C)nc3-c3ccccc3)c2cc1-c1cccc2cn(C)nc12] |
| | Procedure 1; Boronate: (2-methyl-2H-indazol-7-yl)boronic acid |
| | LCMS: m/z = 447 [M + H]⁺ |
| 139^{A} | 7-methoxy-4-(1-methyl-3-phenyl-1H-pyrazol-4-yl)-6-(2-(piperazin-1- |
| | yl)pyridin-4-yl)quinazoline formate |
| | 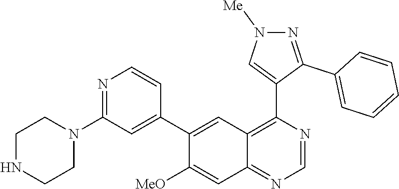 [SMILES: COc1cc2ncnc(-c3cn(C)nc3-c3ccccc3)c2cc1-c1ccnc(N2CCNCC2)c1] |
| | Procedure 1; Boronate: tert-butyl 4-(4-(4,4,5,5-tetramethyl-1,3,2- |
| | dioxaborolan-2-yl)pyridin-2-yl)piperazine-1-carboxylate |
| | LCMS: m/z = 478 [M + H]⁺ |
| 140 | 7-methoxy-6-(1-methyl-1H-pyrrolo[2,3-b]pyridin-5-yl)-4-(1-methyl-3-phenyl- |
| | 1H-pyrazol-4-yl)quinazoline |
| | 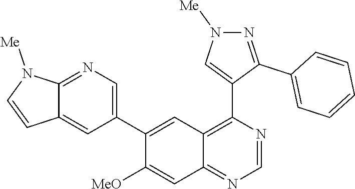 [SMILES: COc1cc2ncnc(-c3cn(C)nc3-c3ccccc3)c2cc1-c1cnc2c(ccn2C)c1] |
| | Procedure 1; Boronate: 1-methyl-5-(4,4,5,5-tetramethyl-1,3,2-dioxaborolan-2- |
| | yl)-1H-pyrrolo[2,3-b]pyridine |
| | LCMS: m/z = 447 [M + H]⁺ |
| 141 | 7-methoxy-6-(1-methyl-1H-indazol-4-yl)-4-(1-methyl-3-phenyl-1H-pyrazol-4- |
| | yl)quinazoline |
| | 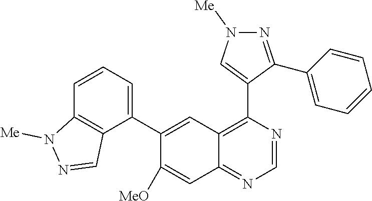 [SMILES: COc1cc2ncnc(-c3cn(C)nc3-c3ccccc3)c2cc1-c1cccc2c1cnn2C] |
| | Procedure 1; Boronate: (1-methyl-1H-indazol-4-yl)boronic acid |
| | LCMS: m/z = 447 [M + H]⁺ |
| 142 | N-cyclopropyl-3-(7-methoxy-4-(1-methyl-3-phenyl-1H-pyrazol-4- |
| | yl)quinazolin-6-yl)benzamide |
| | 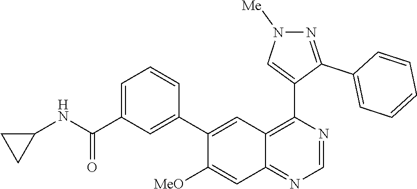 [SMILES: COc1cc2ncnc(-c3cn(C)nc3-c3ccccc3)c2cc1-c1cccc(C(=O)NC2CC2)c1] |
| | Procedure 2; Boronate: N-cyclopropyl-3-(4,4,5,5-tetramethyl-1,3,2- |
| | dioxaborolan-2-yl)benzamide |
| | LCMS: m/z = 476 [M + H]⁺ |
| 143 | 6-(1H-indazol-4-yl)-7-methoxy-4-(1-methyl-3-phenyl-1H-pyrazol-4- |
| | yl)quinazoline |
| | 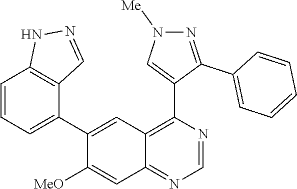 [SMILES: COc1cc2ncnc(-c3cn(C)nc3-c3ccccc3)c2cc1-c1cccc2[nH]ncc12] |
| | Procedure 2; Boronate: 4-(4,4,5,5-tetramethyl-1,3,2-dioxaborolan-2-yl)-1H- |
| | indazole |
| | LCMS: m/z = 433 [M + H]⁺ |
| 144 | 6-(7-methoxy-4-(1-methyl-3-phenyl-1H-pyrazol-4-yl)quinazolin-6- |
| | yl)isoquinolin-1(2H)-one |
| | 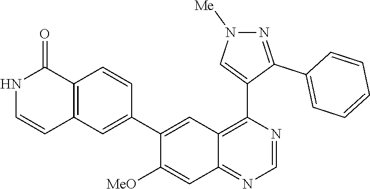 [SMILES: COc1cc2ncnc(-c3cn(C)nc3-c3ccccc3)c2cc1-c1ccc2c(=O)[nH]ccc2c1] |
| | Procedure 2; Boronate: 6-(4,4,5,5-tetramethyl-1,3,2-dioxaborolan-2- |
| | yl)isoquinolin-1(2H)-one |
| | LCMS: m/z = 460 [M + H]⁺ |
| 145 | 7-methoxy-6-(4-methyl-2H-indazol-5-yl)-4-(1-methyl-3-phenyl-1H-pyrazol-4- |
| | yl)quinazoline |
| | 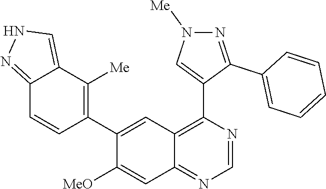 [SMILES: COc1cc2ncnc(-c3cn(C)nc3-c3ccccc3)c2cc1-c1ccc2n[nH]cc2c1C] |
| | Procedure 2; Boronate: (4-methyl-2H-indazol-5-yl)boronic acid |
| | LCMS: m/z = 447 [M + H]⁺ |
| 146 | 7-methoxy-6-(6-methyl-2H-indazol-5-yl)-4-(1-methyl-3-phenyl-1H-pyrazol-4- |
| | yl)quinazoline |
| | 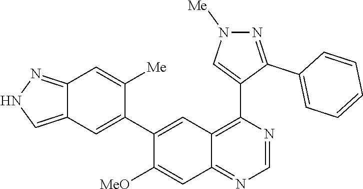 [SMILES: COc1cc2ncnc(-c3cn(C)nc3-c3ccccc3)c2cc1-c1cc2c[nH]nc2cc1C] |
| | Procedure 2; Boronate: (6-methyl-2H-indazol-5-yl)boronic acid |
| | LCMS: m/z = 447 [M + H]⁺ |
| 147 | 7-methoxy-6-(6-methyl-2H-indazol-5-yl)-4-(1-methyl-3-phenyl-1H-pyrazol-4- |
| | yl)quinazoline |
| | 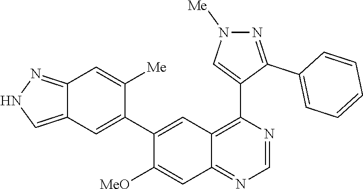 [SMILES: COc1cc2ncnc(-c3cn(C)nc3-c3ccccc3)c2cc1-c1cc2c[nH]nc2cc1C] |
| | Procedure 2; Boronate: 7-methyl-4-(4,4,5,5-tetramethyl-1,3,2-dioxaborolan-2- |
| | yl)-1H-indazole |
| | LCMS: m/z = 447 [M + H]⁺ |

^{A}the crude was purified by Column: Xtimate C18 150*25 mm*5 μm; Mobile phase: A: FA/H₂O =
0.225% v/v; B: MeCN

### Example 148: (4-(7-methoxy-4-(1-methyl-3-phenyl-1H-pyrazol-4-yl)quinazolin-6-yl)-1-methyl-1H-benzo[d]imidazol-2-yl)methanol

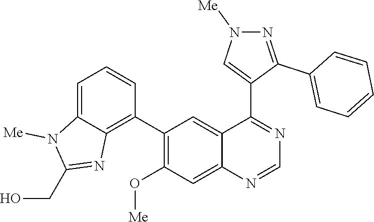 [SMILES: COc1cc2ncnc(-c3cn(C)nc3-c3ccccc3)c2cc1-c1cccc2c1nc(CO)n2C]

Step 1. To a solution of 6-bromo-7-methoxy-4-(1-methyl-3-phenyl-1H-pyrazol-4-yl)quinazoline (Preparation 91, 71.1 mg, 0.18 mmol) and bis(pinacolato)diboron (0.27 mmol) in dioxane (3 mL) was added KOAc (0.54 mmol) and Pd(dppf)Cl₂·DCM (0.018 mmol) under N₂ and the mixture stirred at 100° C. for 16 h. The reaction mixture was diluted with water (5 mL) and extracted with EtOAc (3×4 mL). The combined organics were concentrated by speedvac to afford 7-methoxy-4-(1-methyl-3-phenyl-1H-pyrazol-4-yl)-6-(4,4,5,5-tetramethyl-1,3,2-dioxaborolan-2-yl)quinazoline which was used without further purification.

Step 2. To a solution of 7-methoxy-4-(1-methyl-3-phenyl-1H-pyrazol-4-yl)-6-(4,4,5,5-tetramethyl-1,3,2-dioxaborolan-2-yl)quinazoline (Part 1, 0.18 mmol) and (4-bromo-1-methyl-1H-benzo[d]imidazol-2-yl)methanol (0.27 mmol) in dioxane (3 mL) was added Cs₂CO₃ (2.0 M aq., 0.54 mmol) and Pd(dppf)Cl₂·DCM (0.018 mmol) under N₂ and the mixture stirred at 80° C. for 16 h. The reaction mixture was diluted with water (5 mL) and extracted with EtOAc (3×4 mL). The combined organics were evaporated to dryness and the residue purified by prep-HPLC (Column: Xtimate C18 150*25 mm*5 μm; Mobile Phase: A: FA/H₂O=0.225% v/v; B: MeCN) to give the title compound. LCMS: m/z=477 [M+H]⁺

### Example 149:2-(5-(7-methoxy-4-(1-methyl-3-phenyl-1H-pyrazol-4-yl)quinazolin-6-yl) thiazol-2-yl) propan-2-ol

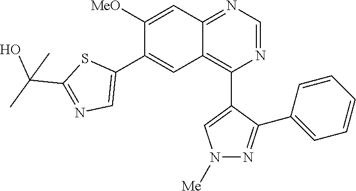 [SMILES: COc1cc2ncnc(-c3cn(C)nc3-c3ccccc3)c2cc1-c1cnc(C(C)(C)O)s1]

A mixture of 6-bromo-7-methoxy-4-(1-methyl-3-phenyl-1H-pyrazol-4-yl)quinazoline (Preparation 91, 80 mg, 0.202 mmol), 5-(tributylstannyl)thiazole (91 mg, 0.243 mmol) and Pd(PPh₃)₂Cl₂ (7.10 mg, 0.010 mmol) in dioxane (1.5 mL) was sparged with N₂ and heated to 90° C. for 5 h. The reaction mixture was concentrated under reduced pressure and purified by HPLC Column: XBridge Shield RP18 OBD Column, 30*150 mm, 5 um; 0-40% (0.1% TFA modified water/MeCN) at 60 mL/min to give the title compound, 94 mg, ¹H NMR (MeOH-d₄) δ: 9.04 (d, 1H), 8.13 (d, 1H), 7.81-7.75 (m, 1H), 7.38-7.33 (m, 2H), 7.24 (dt, 2H), 7.14-7.08 (m, 3H), 4.00 (d, 6H), 1.51 (s, 6H)

### Example 150:6-(1,3-dimethyl-1H-pyrazol-4-yl)-7-methoxy-4-(1-methyl-3-phenyl-1H-pyrazol-4-yl)quinazoline

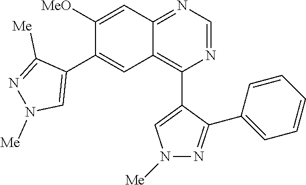 [SMILES: COc1cc2ncnc(-c3cn(C)nc3-c3ccccc3)c2cc1-c1cn(C)nc1C]

To a mixture of 7-methoxy-4-(1-methyl-3-phenyl-1H-pyrazol-4-yl)quinazolin-6-yl trifluoromethane sulfonate (Preparation 116, 35 mg, 0.075 mmol) and 1,3-dimethyl-4-(4,4,5,5-tetramethyl-1,3,2-dioxaborolan-2-yl)-1H-pyrazole (29.3 mg, 0.132 mmol) in dioxane (0.685 mL) and water (0.069 mL) was added K₃PO₄ (32.0 mg, 0.151 mmol) and PdCl₂(dppf):DCM (12.31 mg, 0.015 mmol) and the reaction stirred at 80° C. for 2 h under N₂. The reaction mixture was filtered through a Celite® plug washing through with DCM w/5% MeOH, and the filtrate evaporated under reduced pressure. The residue was purified by silica gel column chromatography (ISCO) to give the title compound, as a brown solid, 24.8 mg, 80%. 1H NMR (500 MHZ, DMSO-d₆) δ 9.12 (d, 1H), 8.23 (d, 1H), 7.63 (d, 1H), 7.50 (d, 1H), 7.45 (d, 1H), 7.32 (d, 2H), 7.24 (q, 3H), 4.01 (dd, 6H), 3.77 (d, 3H), 1.95 (d, 3H).

### Example 151:7-methoxy-6-(7-methoxy-2H-indazol-4-yl)-4-(1-methyl-3-phenyl-1H-pyrazol-4-yl)quinazoline

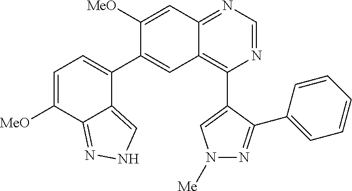 [SMILES: COc1cc2ncnc(-c3cn(C)nc3-c3ccccc3)c2cc1-c1ccc(OC)c2n[nH]cc12]

A mixture of K₂CO₃ (67.8 mg, 0.492 mmol), Pd(dppf)Cl₂ (26.7 mg, 0.033 mmol), 7-methoxy-4-(4,4,5,5-tetramethyl-1,3,2-dioxaborolan-2-yl)-2H-indazole (90 mg, 0.328 mmol) and 6-bromo-7-methoxy-4-(1-methyl-3-phenyl-1H-pyrazol-4-yl)quinazoline (Preparation 91, 194 mg, 0.492 mmol) in H₂O (2 mL) and dioxane (8 mL) was stirred at 80° C. for 2 h under N₂. The mixture was diluted with DCM (100 mL), washed with water (50 mL×2), the organic layer was dried over Na₂SO₄ and concentrated under vacuum. The residue was purified by silica gel column with DCM:MeOH=20:1. The residue was further purified by prep-HPLC: Column: YMC-Actus Triart C18 ExRS, 30*150 mm, 5 μm; Mobile Phase A: Water (10 mmol/L NH₄CO₃+0.1% NH₃·H₂O), Mobile Phase B: MeCN: Flow rate: 60 mL/min; Gradient: 28% B to 47% B in 8 min, to give the title compound (62 mg) as an off-white solid. LCMS: m/z=463 [M+H]⁺. 1HNMR (400 MHZ, DMSO-d₆) δ 13.30 (s, 1H), 9.17 (s, 1H), 8.25 (s, 1H), 7.66 (s, 1H), 7.58-7.49 (m, 2H), 7.29 (tdd, 5H), 6.78 (d, 1H), 6.47 (d, 1H), 3.97 (d, 6H), 3.93 (s, 3H).

### Example 152:5-(7-methoxy-4-(1-methyl-3-phenyl-1H-pyrazol-4-yl)pyrido[3,2-d]pyrimidin-6-yl) isoindolin-1-one

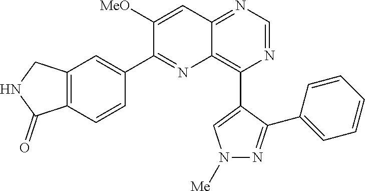 [SMILES: COc1cc2ncnc(-c3cn(C)nc3-c3ccccc3)c2nc1-c1ccc2c(c1)CNC2=O]

To a solution of 6-chloro-7-methoxy-4-(1-methyl-3-phenyl-1H-pyrazol-4-yl)pyrido[3,2-d]pyrimidine (Preparation 242, 200 mg, 0.568 mmol) in dioxane (4.00 mL) and H₂O (1.0 mL) as added 5-(4,4,5,5-tetramethyl-1,3,2-dioxaborolan-2-yl)-2,3-dihydro-1H-isoindol-1-one (176 mg, 0.681 mmol) K₂CO₃ (234 mg, 1.70 mmol) and Pd(dppf)Cl₂·DCM (46.3 mg, 0.057 mmol) and the reaction stirred for 3 h at 80° C. The mixture was concentrated under vacuum, the residue was diluted with water (20 mL) and extracted with EtOAc (2×20 mL). The combined organic layers were washed with brine (20 mL), dried over anhydrous Na₂SO₄ and concentrated under vacuum. The crude product was purified by prep-HPLC Column: Oxbridge Shield RP18 OBD Column, 30*150 mm, 5 μm; Mobile Phase A: Water (10 m mol/L NH₄HCO₃+0.1% NH₃·H₂O), Mobile Phase B: MeCN: Flow rate: 60 mL/min; Gradient: 5% B to 42% B in 10 min, to give the title compound, 12.5 mg, 4.9%, as a yellow solid. LCMS: m/z=449.2 [M+H]⁺, 1H NMR (300 MHz, DMSO-d₆) δ 10.89 (s, 1H), 9.40 (d, 1H), 8.33 (d, 2H), 8.03 (d, 1H), 7.81 (d, 1H), 7.73-7.51 (m, 1H), 7.35 (s, 1H), 7.35 (s, 1H), 5.16 (s, 2H), 3.86 (s, 3H), 3.44 (d, J=3.6 Hz, 1H), 3.29 (s, 2H), 2.34 (s, 2H), 2.22 (s, 3H), 2.14 (s, 3H).

### Examples 153 to 156

The compounds in the following table were prepared from the appropriate pyrido[3,2-d]pyrimidine and boronate ester, following a similar procedure to that described in Example 152.

| Example Number | Name, Structure, Starting Materials (SM), Data |
|---|---|
| 153 | 5-(7-methoxy-4-(1-methyl-3-phenyl-1H-pyrazol-4-yl)pyrido[3,2-d]pyrimidin- |
| | 6-yl)-2-methylisoindolin-1-one |
| | 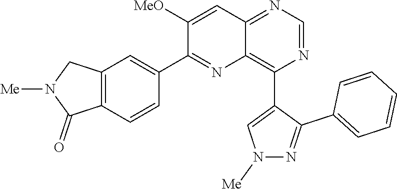 [SMILES: COc1cc2ncnc(-c3cn(C)nc3-c3ccccc3)c2nc1-c1ccc2c(c1)CN(C)C2=O] |
| | SM: 6-chloro-7-methoxy-4-(1-methyl-3-phenyl-1H-pyrazol-4-yl)pyrido[3,2- |
| | d]pyrimidine (Preparation 242) and 2-methyl-5-(4,4,5,5-tetramethyl-1,3,2- |
| | dioxaborolan-2-yl)-2,3-dihydro-1H-isoindol-1-one |
| | Column: X Bridge Shield RP18 OBD Column, 30*150 mm, 5 μm; Mobile |
| | Phase A: Water (10 mmol/L NH₄HCO₃ + 0.1% NH₃•H₂O), Mobile Phase B: |
| | MeCN; Flow rate: 60 mL/min; Gradient: 10% B to 59% B in 10 min |
| | 77.1 mg, 74.1%, as an off-white solid. LCMS: m/z = 463 [M + H]⁺, 1H NMR |
| | (400 MHz, DMSO-d₆) δ 9.16 (s, 1H), 8.48 (s, 1H), 7.89 (s, 1H), 7.74-7.60 |
| | (m, 2H), 7.45-7.38 (m, 3H), 7.35-7.14 (m, 3H), 4.51 (s, 2H), 4.02 (d, 6H), |
| | 3.13 (s, 3H) |
| 154^{A} | 8-(7-methoxy-4-(1-methyl-3-phenyl-1H-pyrazol-4-yl)pyrido[3,2-d]pyrimidin- |
| | 6-yl)-2H-benzo[b][1,4]oxazin-3(4H)-one |
| | 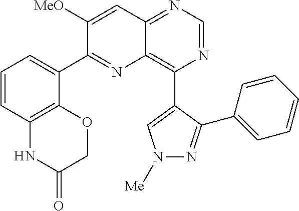 [SMILES: COc1cc2ncnc(-c3cn(C)nc3-c3ccccc3)c2nc1-c1cccc2c1OCC(=O)N2] |
| | SM: 8-(4,4,5,5-tetramethyl-1,3,2-dioxaborolan-2-yl)-2H-benzo[b][1,4]oxazin- |
| | 3-(4H)-one (Preparation 52) and 6-chloro-7-methoxy-4-(1-methyl-3-phenyl- |
| | 1H-pyrazol-4-yl)pyrido[3,2-d]pyrimidine (Preparation 242) |
| | Column: XBridge Shield RP18 OBD Column, 30*150 mm, 5 μm; Mobile |
| | Phase A: Water (10 mmol/L NH₄HCO₃ + 0.1% NH₃•H₂O), Mobile Phase B: |
| | MeCN; Flow rate: 60 mL/min; Gradient: 5% B to 52% B; |
| | 59.8 mg as a white solid. |
| | LCMS: m/z = 465 [M + H]⁺ 1H NMR (400 MHz, DMSO-d₆) δ 10.81 (s, 1H), |
| | 9.11 (s, 1H), 8.46 (s, 1H), 7.80 (s, 1H), 7.37-7.34 (m, 2H), 7.30-7.27 (m, 3H), |
| | 7.01-6.95 (m, 2H), 6.45 (dd, 2.5 Hz, 1H), 4.52 (s, 2H), 4.01 (s, 3H), 3.97 (s, 3H). |
| 155^{A} | 7-methoxy-6-(2-methyl-2H-indazol-4-yl)-4-(1-methyl-3-phenyl-1H-pyrazol-4- |
| | yl)pyrido[3,2-d]pyrimidine |
| | 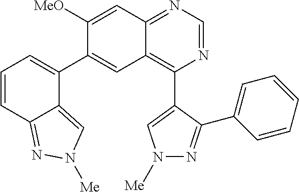 [SMILES: COc1cc2ncnc(-c3cn(C)nc3-c3ccccc3)c2cc1-c1cccc2nn(C)cc12] |
| | SM: 6-chloro-7-methoxy-4-(1-methyl-3-phenyl-1H-pyrazol-4-yl)pyrido[3,2- |
| | d]pyrimidine (Preparation 242) and 2-methyl-7-(4,4,5,5-tetramethyl-1,3,2- |
| | dioxaborolan-2-yl)-2H-indazole |
| | Column: YMC-Actus Triart C18 ExRS, 30*150 mm, 5 μm; Mobile Phase A: |
| | Water (10 mmol/L NH₄CO₃ + 0.1 NH₃•H₂O), Mobile Phase B: MeCN; Flow |
| | rate: 60 mL/min; Gradient: 30% B to 52% B in 8 min |
| | 73.7 mg, 65% as an off-white solid. |
| | LCMS: m/z = 448 [M + H]⁺, 1H NMR (300 MHz, DMSO-d₆) δ 9.03 (s, 1H), |
| | 8.88 (s, 1H), 8.46 (s, 1H), 7.84 (d, 2H), 7.45-7.40 (m, 2H), 7.34-7.28 (m, |
| | 3H), 7.19-7.08 (m, 2H), 4.20 (s, 3H), 3.96 (s, 3H), 3.91 (s, 3H). |
| 156 | 7-ethoxy-6-(2-methyl-2H-indazol-4-yl)-4-(1-methyl-3-phenyl-1H-pyrazol-4- |
| | yl)pyrido[3,2-d]pyrimidine |
| | 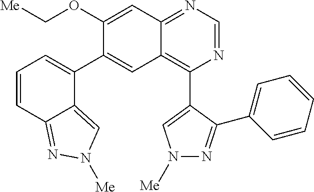 [SMILES: CCOc1cc2ncnc(-c3cn(C)nc3-c3ccccc3)c2cc1-c1cccc2nn(C)cc12] |
| | SM: 6-chloro-7-ethoxy-4-(1-methyl-3-phenyl-1H-pyrazol-4-yl)pyrido[3,2- |
| | d]pyrimidine (Preparation 231) and 2-methyl-7-(4,4,5,5-tetramethyl-1,3,2- |
| | dioxaborolan-2-yl)-2H-indazole |
| | Column: XBridge Shield RP18 OBD Column, 30*150 mm, 5 μm; Mobile |
| | Phase A: Water (10 mmol/L NH₄HCO₃ + 0.1% NH₃•H₂O), Mobile Phase B: |
| | MeCN; Flow rate: 60 mL/min; Gradient: 1% B to 59% B in 10 min |
| | 71.6 mg, 81.2%, as an off-white solid. |
| | LCMS: m/z = 462 [M + H]⁺, 1H NMR (300 MHz, DMSO-d₆) δ 9.01 (s, 1H), |
| | 8.94 (s, 1H), 8.47 (s, 1H), 7.84 (d, 2H), 7.49-7.39 (m, 2H), 7.36-7.26 (m, |
| | 3H), 7.23 (dd, 1H), 7.12 (dd, 1H), 4.31 (q, 2H), 4.22 (s, 3H), 3.91 (s, 3H), 1.25 |
| | (t, 3H). |

^{A}Cs₂CO₃ was used instead of K₂CO₃

### Example 157: methyl (7-methoxy-4-(1-methyl-3-phenyl-1H-pyrazol-4-yl)quinazolin-6-yl)carbamate

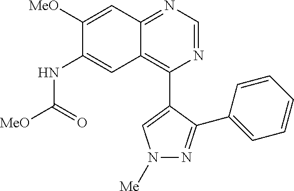 [SMILES: COC(=O)Nc1cc2c(-c3cn(C)nc3-c3ccccc3)ncnc2cc1OC]

Methyl carbonochloridate (25 mg, 0.264 mmol) was added dropwise to 7-methoxy-4-(1-methyl-3-phenyl-1H-pyrazol-4-yl)quinazolin-6-amine (Preparation 190, 80 mg, 0.241 mmol) and TEA (48.6 mg, 0.481 mmol) in DCM (3 mL) at 0° C. and the mixture was stirred at rt for 3 h. The mixture was concentrated under vacuum and the crude product was purified by preparative HPLC: Column: XBridge Prep OBD C18 Column, 30×150 mm 5 um; Mobile Phase A: Water (10 mmol/L NH₄HCO₃+0.1% NH₃·H₂O), Mobile Phase B: MeCN: Flow rate: 60 mL/min; Gradient: 26% B to 41% B in 7 min to give the title compound, 20.5 mg as an off-white solid. LCMS: m/z=390 [M+H]⁺: H NMR (300 MHz, DMSO-d₆): ppm δ 9.03 (s, 1H), 8.88 (s, 1H), 8.35 (s, 1H), 8.17 (s, 1H), 7.41 (s, 1H), 7.33-7.23 (m, 2H), 7.20 (dp, 3H), 4.02 (d, 6H), 3.65 (s, 3H).

### Example 158:7-ethoxy-4-(1-methyl-3-phenyl-1H-pyrazol-4-yl)-N-(tetrahydro-2H-pyran-4-yl)pyrido[3,2-d]pyrimidin-6-amine

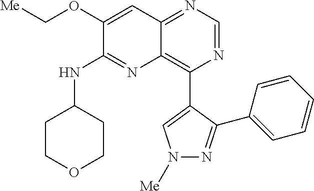 [SMILES: CCOc1cc2ncnc(-c3cn(C)nc3-c3ccccc3)c2nc1NC1CCOCC1]

Cs₂CO₃ (44.3 mg, 0.136 mmol) was added to tetrahydro-2H-pyran-4-amine (27.6 mg, 0.273 mmol), Ruphos Pd(5.70 mg, 0.007 mmol) and 6-chloro-7-ethoxy-4-(1-methyl-3-phenyl-1H-pyrazol-4-yl)pyrido[3,2-d]pyrimidine (Preparation 231, 25 mg, 0.068 mmol) in dioxane (4 mL) and the reaction mixture was stirred at 100° C. for 3 h under N₂. The mixture was diluted with EtOAc (100 mL), washed with brine (50 mL×2), the organic layer was dried over Na₂SO₄ and concentrated under vacuum. The residue was purified by Prep-TLC with DCM:MeOH=15:1. The product was further purified by prep-HPLC (Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN: Flow rate: 60 mL/min; Gradient: 32% B to 42% B in 8 min, to give the title compound, 13.1 mg as an off-white solid. LCMS: m/z=431 [M+H]⁺1H NMR (300 MHz, DMSO-d₆) δ 8.66 (s, 1H), 8.48 (s, 1H), 7.44-7.30 (m, 2H), 7.28-7.19 (m, 4H), 6.81 (d, 1H), 4.28 (q, 2H), 3.98 (s, 3H), 3.90 (s, 2H), 3.39 (d, 1H), 1.72 (d, 2H), 1.66-1.56 (m, 1H), 1.59-1.49 (m, 1H), 1.44 (t, 3H).

### Example 159:4-(4-(3-(2-fluorophenyl)-1-methyl-1H-pyrazol-4-yl)-7-methoxyquinazolin-6-yl)morpholine

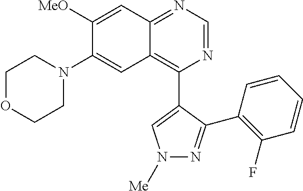 [SMILES: COc1cc2ncnc(-c3cn(C)nc3-c3ccccc3F)c2cc1N1CCOCC1]

To a solution of 4-(3-(2-fluorophenyl)-1-methyl-1H-pyrazol-4-yl)-7-methoxyquinazolin-6-yl trifluoromethanesulfonate (Preparation 118, 240 mg, 0.5 mmol) in dioxane (10 mL) was added morpholine (108 mg, 1.24 mmol), Cs₂CO₃ (645 mg, 1.98 mmol) and BINAP Pd G3 (46.2 mg, 0.05 mmol) under N₂ and the reaction was stirred at 100° C. for 3 h. The cooled reaction mixture was diluted with water (20 mL), extracted with EtOAc (2×20 mL) and the organic layers combined. The organic solution was washed with brine (20 mL), dried over anhydrous Na₂SO₄ and concentrated under vacuum. The crude product was purified by Prep-HPLC Column: YMC-Altus Traits C18, 30*150 mm, 5 μm; Mobile Phase A: Water (10 m mol/L NH₂HCO₃+0.1% NH₃·H₂O), Mobile Phase B: MeCN: Flow rate: 60 mL/min; Gradient: 19% B to 49% B in 7 min, to give the title compound, 42.1 mg, 20.2% as an off-white solid. LCMS: m/z=420, 421 [M+H]⁺1H NMR (400 MHZ, DMSO-d₆) δ 8.94 (s, 1H), 8.39 (s, 1H), 7.65 (td, 1H), 7.32 (d, 2H), 7.24 (td, 1H), 7.08-6.82 (m, 2H), 4.06 (s, 3H), 3.97 (s, 3H), 3.71-3.62 (m, 4H), 2.71 (t, 4H).

### Example 160:4-(7-methoxy-4-(1-methyl-3-phenyl-1H-pyrazol-4-yl)quinazolin-6-yl)morpholine

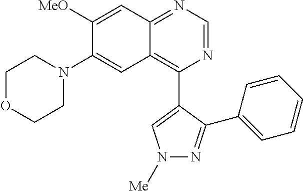 [SMILES: COc1cc2ncnc(-c3cn(C)nc3-c3ccccc3)c2cc1N1CCOCC1]

A mixture of 7-methoxy-4-(1-methyl-3-phenyl-1H-pyrazol-4-yl)quinazolin-6-yl trifluoromethane sulfonate (Preparation 116, 50 mg, 0.108 mmol), Cs₂CO₃ (70.2 mg, 0.215 mmol) and Xantphos Pd G3 (10.21 mg, 0.011 mmol) in dioxane (1.08 mL) was purged with N₂ and stirred at 100° C. overnight. The cooled reaction mixture was filtered through silica gel, eluting with DCM:MeOH (95:5) and the filtrate concentrated in vacuo. The crude product was purified by ISCO Combiflash to give the title compound as a beige solid, 23 mg, 53% yield. LCMS m/z=402 [M+H]⁺1H NMR (500 MHz, DMSO-d₆) δ 9.12-8.97 (m, 1H), 8.28 (t, 1H), 7.39-7.17 (m, 6H), 6.90 (t, 1H), 4.05 (t, 3H), 3.99 (t, 3H), 3.70-3.59 (m, 4H), 2.64 (d, 4H).

### Example 161:6-(7-methoxy-4-(1-methyl-3-phenyl-1H-pyrazol-4-yl)quinazolin-6-yl)-2-oxa-6-azaspiro[3.3]heptane

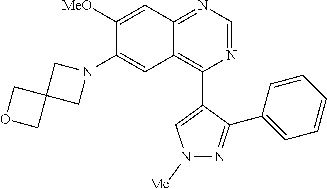 [SMILES: COc1cc2ncnc(-c3cn(C)nc3-c3ccccc3)c2cc1N1CC2(COC2)C1]

A mixture of 7-methoxy-4-(1-methyl-3-phenyl-1H-pyrazol-4-yl)quinazolin-6-yl trifluoromethane sulfonate (Preparation 116, 40 mg, 0.086 mmol), Cs₂CO₃ (56.1 mg, 0.172 mmol) and Xantphos Pd G3 (8.17 mg, 0.0086 mmol) in dioxane (0.86 mL) was purged with N₂ and stirred at 100° C. The cooled reaction was filtered through silica gel eluting with DCM w/5% MeOH and the filtrate was concentrated in vacuo. The residue was purified by reverse phase ISCO, eluting with 5-70% MeCN w/0.1% TFA in H₂O, the product containing fractions neutralized with sat. Na₂CO₃ and extracted with DCM (w 5% MeOH). The combined organic extracts were dried over anhydrous Na₂SO₄, filtered and solvent removed under reduced pressure to give the title compound as a yellow solid, 19.3 mg, 54%. 1H NMR (500 MHz, DMSO-d₆) δ 8.93 (s, 1H), 8.22 (s, 1H), 7.25 (dd, 6H), 6.29 (d, J=3.2 Hz, 1H), 4.64 (s, 4H), 4.04 (s, 3H), 3.95 (s, 3H), 3.81 (s, 4H).

### Example 162:7-ethoxy-4-(1-methyl-3-phenyl-1H-pyrazol-4-yl)-6-(4-methylpiperazin-1-yl)quinazoline

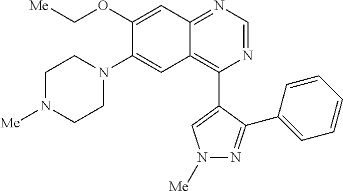 [SMILES: CCOc1cc2ncnc(-c3cn(C)nc3-c3ccccc3)c2cc1N1CCN(C)CC1]

To a mixture of 6-bromo-7-ethoxy-4-(1-methyl-3-phenyl-1H-pyrazol-4-yl)quinazoline (Preparation 90, 50 mg, 0.122 mmol) and 1-methylpiperazine (10.6 mg, 0.122 mmol) in dioxane (3 mL) was added Ruphos Pd(10.2 mg, 0.012 mmol) and Cs₂CO₃ (79.5 mg, 0.244 mmol) and the reaction mixture was stirred at 100° C. for 4 h under N₂. The mixture was diluted with water (20 mL), extracted with EtOAc (2×20 mL) and the organic layers combined. The solution was washed with brine (20 mL), dried over anhydrous Na₂SO₄ and concentrated under vacuum. The product was purified by TLC eluting with DCM:MeOH (10:1). The product was further purified by Prep-HPLC Column: XBridge Shield RP18 OBD Column, 30*150 mm, 5 um; Mobile Phase A: Water (10 mmol/l NH₄HCO₃+0.1% NH₃·H₂O), Mobile Phase B: MeCN; Flow rate: 60 mL/min; Gradient: 28% B to 45% B in 8 min, to afford the title compound, 15.5 mg, 30.6%. LCMS: m/z=429 [M+H]⁺, 1H NMR (300 MHz, DMSO-d₆) δ 9.03 (s, 1H), 8.26 (s, 1H), 7.31-7.17 (m, 6H), 6.86 (s, 1H), 4.22 (q, 2H), 4.03 (s, 3H), 3.61 (d, 4H), 2.64 (d, 4H), 1.41 (t, 3H). (d, 4H), 1.41 (t, 3H).

### Example 163:4-(7-ethoxy-4-(1-methyl-3-phenyl-1H-pyrazol-4-yl)quinazolin-6-yl)morpholine

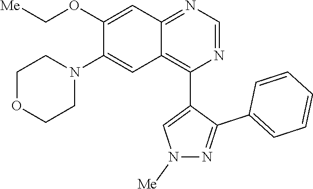 [SMILES: CCOc1cc2ncnc(-c3cn(C)nc3-c3ccccc3)c2cc1N1CCOCC1]

The title compound was obtained, 15.5 mg, 30.6% yield, from 6-bromo-7-ethoxy-4-(1-methyl-3-phenyl-1H-pyrazol-4-yl)quinazoline (Preparation 90) and morpholine, following the procedure described in Example 162. LCMS m/z=416 [M+H]⁺. 1H NMR (300 MHZ, DMSO-d₆) δ 9.03 (s, 1H), 8.26 (s, 1H), 7.31-7.17 (m, 6H), 6.86 (s, 1H), 4.22 (q, 2H), 4.03 (s, 3H), 3.61 (d, 4H), 2.64 (d, 4H), 1.41 (t, 3H).

### Example 164: (S)-1-(7-methoxy-4-(1-methyl-3-phenyl-1H-pyrazol-4-yl)quinazolin-6-yl) piperidin-3-ol

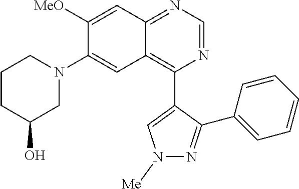 [SMILES: COc1cc2ncnc(-c3cn(C)nc3-c3ccccc3)c2cc1N1CCC[C@H](O)C1]

To a solution of 6-bromo-7-methoxy-4-(1-methyl-3-phenyl-1H-pyrazol-4-yl)quinazoline (Preparation 91, 100 mg, 0.253 mmol) in dioxane (8 mL) was added (3S)-piperidin-3-ol (76.6 mg, 0.758 mmol), Cs₂CO₃ (492 mg, 1.51 mmol) and PEPPSI Pd-Ipent-O-Picoline (21.2 mg, 25.2 μmol) and the reaction was stirred for 16 h at 120° C. The cooled mixture was diluted with water (20 mL) extracted with EtOAc (2×20 mL) and the organic layers combined. The organic solution was washed with brine (20 mL) dried over anhydrous Na₂SO₄ and concentrated under vacuum. The residue was purified by prep-HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 12% B to 46% B in 7 min; to give the title compound, 19.3 mg, 18.5% as a yellow solid. LCMS: m/z=416 [M+H]⁺. 1H NMR (400 MHZ, DMSO-d₆) δ 9.03 (s, 1H), 8.25 (s, 1H), 7.33-7.12 (m, 6H), 6.88 (s, 1H), 4.71 (d, 1H), 4.03 (s, 3H), 3.97 (s, 3H), 3.50 (dq, 1H), 3.17 (d, 1H), 2.73 (d, 1H), 2.11 (dd, 1H), 1.98 (t, 1H), 1.84 (d, 1H), 1.61 (d, 1H), 1.41 (q, 1H), 1.19-1.05 (m, 1H)

### Examples 165 to 170

The compounds in the following table were prepared from the appropriate bromo quinazoline and amine following a similar procedure to that described in Example 164.

| Example No | Name/Structure/Starting material/Data |
|---|---|
| 165 | (R)-1-(7-methoxy-4-(1-methyl-3-phenyl-1H-pyrazol-4-yl)quinazolin-6- |
| | yl)piperidin-3-ol |
| | 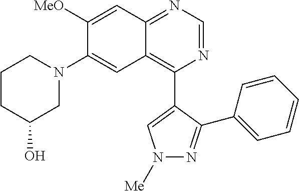 [SMILES: COc1cc2ncnc(-c3cn(C)nc3-c3ccccc3)c2cc1N1CCC[C@@H](O)C1] |
| | SM: 6-bromo-7-methoxy-4-(1-methyl-3-phenyl-1H-pyrazol-4-yl)quinazoline |
| | (Preparation 91) and (3R)-piperidin-3-ol |
| | yellow solid, 16.9 mg, 16% yield |
| | prep-HPLC (Column: YMC-Actus Triart C18 ExRS, 30*150 mm, 5 μm; |
| | Mobile Phase A: Water (10 mmol/L NH₄HCO₃ + 0.1% NH₃•H₂O), Mobile |
| | Phase B: MeCN; Flow rate: 60 mL/min; Gradient: 23% B to 53% B in 7 |
| | min;. LCMS m/z = 416 [M + H]⁺ 1H NMR (300 MHz, DMSO-d₆) δ 8.90 (s, |
| | 1H), 8.12 (s, 1H), 7.21-7.02 (m, 6H), 6.75 (s, 1H), 4.57 (d, 1H), 3.87 (d, |
| | 6H), 3.37 (dq, 1H), 3.05 (d, 1H), 2.61 (d, 1H), 2.07-1.92 (m, 1H), 1.91- |
| | 1.78 (m, 1H), 1.71 (d, 1H), 1.49 (d, 1H), 1.28 (d, 1H), 1.10-0.88 (m, 1H). |
| 166^{A} | 4-(4-(3-(2,4-difluorophenyl)-1-methyl-1H-pyrazol-4-yl)-7- |
| | methoxyquinazolin-6-yl)morpholine |
| | 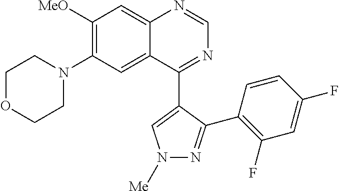 [SMILES: COc1cc2ncnc(-c3cn(C)nc3-c3ccc(F)cc3F)c2cc1N1CCOCC1] |
| | SM: morpholine and 6-bromo-4-(3-(2,4-difluorophenyl)-1-methyl-1H- |
| | pyrazol-4-yl)-7-methoxyquinazoline (Preparation 96) |
| | 33.8 mg, as a light yellow solid |
| | prep-HPLC Column: YMC-Actus Triart C18 ExRS, 30*150 mm, 5 μm; |
| | Mobile Phase A: Water (10 mmol/L NH₄HCO₃ + 0.1% NH₃•H₂O), Mobile |
| | Phase B: MeCN; Flow rate: 60 mL/min; Gradient: 25% B to 55% B in 7 min, |
| | LCMS m/z = 438 [M + H]⁺. 1H NMR (300 MHz, DMSO-d₆) δ 8.91 (s, 1H), |
| | 8.39 (s, 1H), 7.73-7.59 (m, 1H), 7.30 (s, 1H), 7.13 (td, 1H), 7.13-6.98 (m, |
| | 3H), 4.03 (s, 3H), 3.96 (s, 3H), 3.67 (t, 4H), 2.77 (t, 4H). |
| 167^{A} | 3-(7-ethoxy-4-(1-methyl-3-phenyl-1H-pyrazol-4-yl)quinazolin-6-yl)-6-oxa- |
| | 3-azabicyclo[3.1.1]heptane |
| | 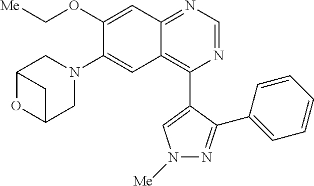 [SMILES: CCOc1cc2ncnc(-c3cn(C)nc3-c3ccccc3)c2cc1N1CC2CC(C1)O2] |
| | SM: 6-bromo-7-ethoxy-4-(1-methyl-3-phenyl-1H-pyrazol-4-yl)quinazoline |
| | (Preparation 90) and 6-oxa-3-azabicyclo[3.1.1]heptane |
| | yellow solid, 26.2 mg, 31% yield |
| | Column: XBridge Shield RP18 OBD Column, 30*150 mm, 5 μm; Mobile |
| | Phase A: Water (10 mmol/L NH₄HCO₃ + 0.1% NH₃•H₂O), Mobile Phase B: |
| | MeCN; Flow rate: 60 mL/min; Gradient: 32% B to 42% B in 8 min.. LCMS: |
| | m/z = 428 [M + H]⁺, 1H NMR (400 MHz, DMSO-d₆) δ 8.97 (s, 1H), 8.25 (s, |
| | 1H), 7.32-7.24 (m, 3H), 7.21 (dt, 3H), 6.91 (s, 1H), 4.50 (d, 2H), 4.25 (q, |
| | 2H), 4.02 (s, 3H), 3.45 (d, 2H), 3.13 (d, 2H), 2.99 (q, 1H), 2.05 (d, 1H), 1.42 |
| | (t, 3H). |
| 168^{A,B} | 4-(4-(1-(2,2-difluoroethyl)-3-phenyl-1H-pyrazol-4-yl)-7-methoxyquinazolin- |
| | 6-yl)morpholine |
| | 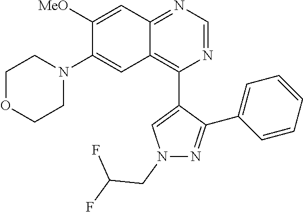 [SMILES: COc1cc2ncnc(-c3cn(CC(F)F)nc3-c3ccccc3)c2cc1N1CCOCC1] |
| | SM: 6-bromo-4-(1-(2,2-difluoroethyl)-3-phenyl-1H-pyrazol-4-yl)-7- |
| | methoxyquinazoline (Preparation 157) and morpholine |
| | off-white solid, 78.5 mg, 55%, |
| | prep-HPLC: Column: XBridge Shield RP18 OBD Column, 30*150 mm, |
| | 5 μm; Mobile Phase A: Water (10 mmol/L NH₄HCO₃ + 0.1% NH₃•H₂O), |
| | Mobile Phase B: MeCN; Flow rate: 60 mL/min; Gradient: 23% B to 56% B |
| | in 7 min; LCMS m/z = 452 [M + H]⁺. 1H NMR (400 MHz, DMSO-d₆) δ ppm |
| | 9.06 (s, 1H), 8.37 (s, 1H), 7.34-7.23 (m, 6H), 6.90 (s, 1H), 6.56 (tt, 1H), 4.84 |
| | (dt, 2H), 3.98 (s, 3H), 3.64 (m, 4H), 2.74 (m, 4H). |
| 169 | 4-(4-(1-ethyl-3-phenyl-1H-pyrazol-4-yl)-7-methoxyquinazolin-6- |
| | yl)morpholine |
| | 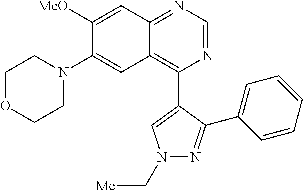 [SMILES: CCn1cc(-c2ncnc3cc(OC)c(N4CCOCC4)cc23)c(-c2ccccc2)n1] |
| | SM: 6-bromo-4-(1-ethyl-3-phenyl-1H-pyrazol-4-yl)-7-methoxyquinazoline |
| | (Preparation 94) and morpholine |
| | prep-HPLC (Column: XBridge Shield RP18 OBD Column, 30*150 mm, |
| | 5 μm; Mobile Phase A: Water (10 mmol/L NH₄HCO₃ + 0.1% NH₃•H₂O), |
| | Mobile Phase B: MeCN; Flow rate: 60 mL/min; Gradient: 6% B to 57% B in |
| | 10 min |
| | yellow solid (20.2 mg, 10%). LCMS m/z = 416 [M + H]⁺ |
| | 1H NMR (300 MHz, DMSO-d₆) δ 9.04 (s, 1H), 8.32 (s, 1H), 7.34-7.23 (m, |
| | 4H), 7.23-7.17 (m, 3H), 6.88 (s, 1H), 4.32 (q, 2H), 3.96 (s, 3H), 3.61 (d, |
| | 4H), 2.61 (t, 4H), 1.52 (t, 3H) |
| 170 | 4-(7-ethoxy-4-(1-ethyl-3-phenyl-1H-pyrazol-4-yl)quinazolin-6- |
| | yl)morpholine |
| | 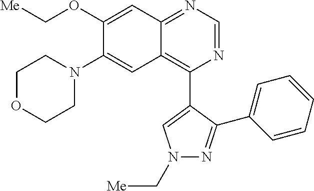 [SMILES: CCOc1cc2ncnc(-c3cn(CC)nc3-c3ccccc3)c2cc1N1CCOCC1] |
| | SM: 6-bromo-7-ethoxy-4-(1-ethyl-3-phenyl-1H-pyrazol-4-yl)quinazoline |
| | (Preparation 93) and morpholine |
| | prep-HPLC (Column: Xselect CSH C18 OBD Column 30*150 mm 5 μm, n; |
| | Mobile Phase A: Water (0.1% FA), Mobile Phase B: MeCN; Flow rate: |
| | 6.22 mL/min; Gradient: 35% B to 50% B in 8 min, 50% B; |
| | as a off-white solid (14.1 mg, 7%). LCMS m/z = 430 [M + H]⁺ |
| | 1H NMR (300 MHz, DMSO-d₆) δ 9.02 (s, 1H), 8.31 (s, 1H), 7.32-7.17 (m, |
| | 7H), 6.86 (s, 1H), 4.32 (q, 2H), 4.22 (q, 2H), 3.61 (t, 4H), 2.63 (t, 4H), 1.52 |
| | (t, 3H), 1.40 (t, 3H). |

^{A}MeCN was used instead of dioxane
^{B}K₂CO₃ was used instead of Cs₂CO₃

### Example 171:4-(4-(1-ethyl-3-(4-fluorophenyl)-1H-pyrazol-4-yl)-7-methoxyquinazolin-6-yl)morpholine

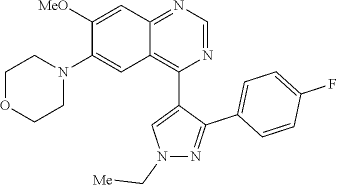 [SMILES: CCn1cc(-c2ncnc3cc(OC)c(N4CCOCC4)cc23)c(-c2ccc(F)cc2)n1]

A mixture of 6-bromo-4-(1-ethyl-3-(4-fluorophenyl)-1H-pyrazol-4-yl)-7-methoxyquinazoline (Preparation 158, 50.0 mg, 0.117 mmol). Cs₂CO₃ (76.7 mg, 0.234 mmol). Brettphos Pd G3 (10.6 mg, 0.012 mmol), morpholine (10.1 mg, 0.117 mmol) and dioxane (5 mL) was stirred for 1 h at 80° C. under N₂. The cooled reaction was then quenched by the addition of H₂O, the solution was extracted with EtOAc (3×10 mL) and the combined organic extracts concentrated under vacuum. The residue was purified by prep-HPLC Column: YMC-Actus Triart C18 ExRS, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 31% B to 51% B in 8 min, to give the title compound, 20.7 mg, 41.5% as a light yellow solid. LCMS m/z=434 [M+H]^{+ 1}NMR (300 MHz, DMSO-d₆) δ 9.03 (s, 1H), 8.34 (s, 1H), 7.31 (dt, 3H), 7.13-7.00 (m, 2H), 6.91 (s, 1H), 4.31 (q, 2H), 3.98 (s, 3H), 3.65 (d, 1H), 3.65 (s, 3H), 1.51 (t, 2H).

### Example 172 and 173: (3S,4S)-4-fluoro-1-(7-methoxy-4-(1-methyl-3-phenyl-1H-pyrazol-4-yl)quinazolin-6-yl) piperidin-3-ol and (3R,4R)-4-fluoro-1-(7-methoxy-4-(1-methyl-3-phenyl-1H-pyrazol-4-yl) quinazolin-6-yl) piperidin-3-ol

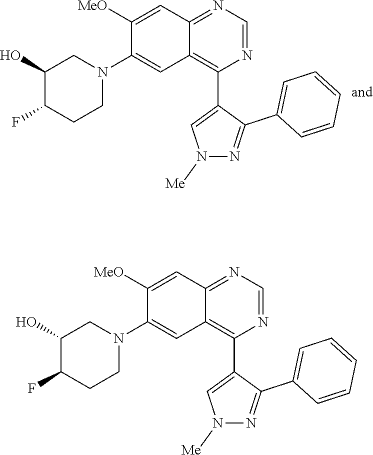 [SMILES: COc1cc2ncnc(-c3cn(C)nc3-c3ccccc3)c2cc1N1CC[C@H](F)[C@@H](O)C1 ; COc1cc2ncnc(-c3cn(C)nc3-c3ccccc3)c2cc1N1CC[C@@H](F)[C@H](O)C1]

A mixture of trans-rac-4-fluoropiperidin-3-ol (60 mg, 0.5 mmol), 6-bromo-7-methoxy-4-(1-methyl-3-phenyl-1H-pyrazol-4-yl)quinazoline (Preparation 91, 200 mg, 0.5 mmol), BrettPhos Pd G3 (39.3 mg, 0.05 mmol) and Cs₂CO₃ (329 mg, 1 mmol) in dioxane (5 mL) was stirred at 100° C. under N₂ for 10 h. Water was added, the mixture was extracted with EtOAc and the combined organic phases concentrated in vacuo. The residue was purified by prep-HPLC (Column: Xselect CSH C18 OBD Column 30*150 mm 5 μm, n: Mobile Phase A: Water (0.1% FA), Mobile Phase B: MeCN: Flow rate: 60 mL/min; Gradient: 22% B to 32% B in 8 min, to afford trans-rac-4-fluoro-1-(7-methoxy-4-(1-methyl-3-phenyl-1H-pyrazol-4-yl)quinazolin-6-yl) piperidin-3-ol (30 mg) as yellow solid. LCMS: m/z=434 [M+H]⁺. This compound, 30 mg was further purified by chiral prep-HPLC (Column: Column: Lux 5 um Amylose-1, 5*25 cm, 10 μm; Mobile Phase A: Hex (0.5% 2M NH₃—MeOH), Mobile Phase B: IPA: Flow rate: 20 mL/min; Gradient: 40% isocratic to give

Peak 1, Example 172, (3S,4S)-4-fluoro-1-(7-methoxy-4-(1-methyl-3-phenyl-1H-pyrazol-4-yl)quinazolin-6-yl) piperidin-3-ol or (3R,4R)-4-fluoro-1-(7-methoxy-4-(1-methyl-3-phenyl-1H-pyrazol-4-yl)quinazolin-6-yl) piperidin-3-ol as a white solid (2.1 mg). LCMS: m/z=434 [M+H]⁺ 1H NMR (300 MHz, DMSO-d₆) δ 9.03 (s, 1H), 8.25 (s, 1H), 7.29-7.22 (m, 6H), 6.89 (s, 1H), 5.31 (d, 1H), 4.33-4.17 (m, 1H), 4.00 (m, 6H), 3.56 (s, 1H), 3.19 (m, 1H), 2.79 (d, 1H), 2.32 (t, 1H), 2.12 (t, 1H), 1.99 (s, 1H), 1.64-1.51 (m, 1H)

Further elution provided Peak 2, 4.8 mg, as a white solid, Example 173, (3R,4R)-4-fluoro-1-(7-methoxy-4-(1-methyl-3-phenyl-1H-pyrazol-4-yl)quinazolin-6-yl) piperidin-3-ol or (3S,4S)-4-fluoro-1-(7-methoxy-4-(1-methyl-3-phenyl-1H-pyrazol-4-yl)quinazolin-6-yl) piperidin-3-ol. LCMS: m/z=434 [M+H]⁺1H NMR (300 MHz, DMSO-d₆) δ 9.03 (s, 1H), 8.25 (s, 1H), 7.29-7.19 (m, 6H), 6.88 (s, 1H), 5.31 (d, 1H), 4.20-4.40 (m, 1H) 4.00 (m, 6H), 3.52 (s, 1H), 3.22 (s, 1H), 2.78 (s, 1H), 2.33 (m, 1H), 2.12 (m, 1H), 1.99 (s, 1H), 1.58 (s, 1H).

### Example 174: (S)-6-(2,4-dimethylpiperazin-1-yl)-7-ethoxy-4-(1-methyl-3-phenyl-1H-pyrazol-4-yl)quinazoline

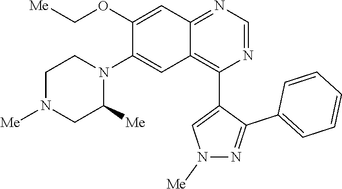 [SMILES: CCOc1cc2ncnc(-c3cn(C)nc3-c3ccccc3)c2cc1N1CCN(C)C[C@@H]1C]

Part 1: TFA (2 mL) was added to tert-butyl(S)-4-(7-ethoxy-4-(1-methyl-3-phenyl-1H-pyrazol-4-yl)quinazolin-6-yl)-3-methylpiperazine-1-carboxylate (Preparation 188, 60 mg, 0.113 mmol) in DCM (6 mL) at rt and the reaction was stirred for 2 h. The mixture was concentrated under vacuum and the residue was purified by Prep-HPLC as following condition: Column: XBridge Shield RP18 OBD Column, 30*150 mm, 5 um; Mobile Phase A: Water (10 mmol/L NH₂HCO₃+0.1% NH₃·H₂O), Mobile Phase B: MeCN; Flow rate: 60 mL/min; Gradient: 20% B to 40% B in 8 min; to give(S)-7-ethoxy-4-(1-methyl-3-phenyl-1H-pyrazol-4-yl)-6-(2-methylpiperazin-1-yl)quinazoline as a yellow solid, 8 mg, 16.5%.

Part 2: NaBH(OAc)₃ (178 mg, 0.840 mmol) was added to(S)-7-ethoxy-4-(1-methyl-3-phenyl-1H-pyrazol-4-yl)-6-(2-methylpiperazin-1-yl)quinazoline (180 mg, 0.420 mmol) and HCHO (94.0 mg, 2.09 mmol) in DCM (10 mL) at 0° C. and the reaction was stirred at rt for 2 h. The mixture was concentrated under vacuum and the residue was purified by Prep-HPLC Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 30% B to 44% B in 8 min, to give the title compound, 62.5 mg as a yellow solid. LCMS m/z=443 [M+H]^{+ 1}H NMR (400 MHZ, DMSO-d₆) δ 9.03 (s, 1H), 8.20 (s, 1H), 7.29-7.22 (m, 3H), 7.22-7.16 (m, 3H), 6.82 (s, 1H), 4.30-4.11 (m, 2H), 4.03 (s, 3H), 3.77 (s, 1H), 2.84 (t, 1H), 2.51 (s, 1H), 2.36 (s, 1H), 2.24 (d, 1H), 2.13-2.09 (m, 4H), 1.99 (d, 1H), 1.40 (t, 3H), 0.63 (d, 3H).

### Example 175:6-(7-ethoxy-4-(1-methyl-3-phenyl-1H-pyrazol-4-yl)pyrido[3,2-d]pyrimidin-6-yl)-2-oxa-6-azaspiro[3.3]heptane

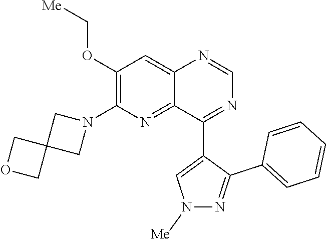 [SMILES: CCOc1cc2ncnc(-c3cn(C)nc3-c3ccccc3)c2nc1N1CC2(COC2)C1]

PEPPSI Pd-Ipent-O-Picoline (18.3 mg, 0.218 mmol) and Cs₂CO₃ (106 mg, 0.327 mmol) were added to 6-chloro-7-ethoxy-4-(1-methyl-3-phenyl-1H-pyrazol-4-yl)pyrido[3,2-d]pyrimidine (Preparation 231, 80 mg, 0.218 mmol) and 2-oxa-6-azaspiro[3.3]heptane (25.8 mg, 0.261 mmol) in dioxane (5 mL) at rt. The reaction mixture was heated to 100° C. for 16 h under N₂, then diluted with EtOAc (100 mL), washed with water (3×100 mL) and brine (100 mL). The organic layer was dried over Na₂SO₄, filtered and evaporated to dryness. The crude product was purified by prep-TLC (20:1 DCM/MeOH) followed by prep-HPLC YMC-Actus Triart C18 ExRS, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 14% B to 44% B in 7 min to afford the title compound as a white solid (12.6 mg, 13%). LCMS m/z=429 [M+H]⁺. ¹H NMR (400 MHZ, DMSO-d₆) δ 8.67 (s, 1H), 8.61 (s, 1H), 7.43-7.32 (m, 2H), 7.32-7.21 (m, 4H), 4.70 (s, 4H), 4.34-4.12 (m, 6H), 4.02 (s, 3H), 1.43 (t, 3H).

### Example 176:1-(7-methoxy-4-(1-methyl-3-phenyl-1H-pyrazol-4-yl)quinazolin-6-yl)-3-methylimidazolidin-2-one

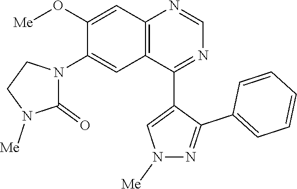 [SMILES: COc1cc2ncnc(-c3cn(C)nc3-c3ccccc3)c2cc1N1CCN(C)C1=O]

A mixture of 7-methoxy-4-(1-methyl-3-phenyl-1H-pyrazol-4-yl)quinazolin-6-yl trifluoro methanesulfonate (Preparation 116, 50 mg, 0.108 mmol), Cs₂CO₃ (70.2 mg, 0.215 mmol), 1-methylimidazolidin-2-one (21.56 mg, 0.215 mmol) and XantPhos Pd G3 (10.2 mg, 0.0108 mmol) in dioxane (1.1 mL) was stirred at 100° C. under N₂ overnight. The mixture was cooled, filtered through a silica gel plug (5% DCM/MeOH) and purified by Combiflash Isco, to yield the title compound as a beige solid (4.9 mg, 11%). LCMS m/z=415 [M+H]⁺. ¹H NMR (500 MHz, DMSO-d₆) δ 9.10 (d, 1H), 8.24 (s, 1H), 7.71 (d, 1H), 7.46 (s, 1H), 7.36-7.18 (m, 5H), 4.04 (s, 3H), 4.00 (s, 3H), 3.49 (t, 2H), 3.40 (t, 2H), 2.74 (d, 3H).

### Example 177: (2S,3S)—N-(7-methoxy-4-(1-methyl-3-phenyl-1H-pyrazol-4-yl)quinazolin-6-yl)-2,3-dimethylmorpholine-4-carboxamide

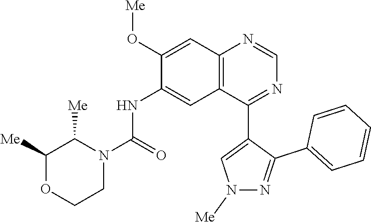 [SMILES: COc1cc2ncnc(-c3cn(C)nc3-c3ccccc3)c2cc1NC(=O)N1CCO[C@@H](C)[C@@H]1C]

To a solution of 7-methoxy-4-(1-methyl-3-phenyl-1H-pyrazol-4-yl)quinazolin-6-amine (Preparation 190, 50.0 mg, 0.151 mmol) and pyridine (23.9 mg, 0.302 mmol) in DCM (1 mL) was added 4-nitrophenyl chloroformate (45.6 mg, 0.226 mmol). The reaction mixture was stirred at 25° C. for 1 h and then added to the HCl salt of (2S,3S)-2,3-dimethylmorpholine (91.5 mg, 0.604 mmol) and TEA (30.5 mg, 0.302 mmol) in DCM (1 mL). The reaction mixture was stirred at 45° C. for 12 h, then concentrated under reduced pressure. The residue was purified by prep-HPLC column: Phenomenex Gemini-NX C18 75*30 mm*3 um; mobile phase: [water (0.05% NH₃H₂O+10 mM NH₂HCO₃)—MeCN]; B %: 15%-45%, 8 min to give the title compound as a yellow solid (15.0 mg, 19%). LCMS m/z=473 [M+H]⁺. ¹H-NMR (400 MHZ, DMSO-d₆) δ ppm 9.03 (s, 1H), 8.36 (s, 1H), 8.18 (s, 1H), 7.91 (s, 1H), 7.43 (s, 1H), 7.32-7.26 (m, 2H), 7.23-7.17 (m, 3H), 4.06 (s, 3H), 4.04 (s, 3H), 3.91-3.82 (m, 1H), 3.79-3.69 (m, 2H), 3.64 (d, 1H), 3.55 (dd, 1H), 3.19 (dt, 1H), 1.23 (t, 6H).

### Example 178: (2S,5S)—N-(7-methoxy-4-(1-methyl-3-phenyl-1H-pyrazol-4-yl)quinazolin-6-yl)-2,4,5-trimethylpiperazine-1-carboxamide

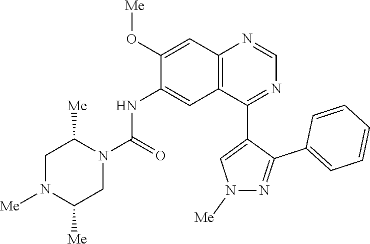 [SMILES: COc1cc2ncnc(-c3cn(C)nc3-c3ccccc3)c2cc1NC(=O)N1C[C@H](C)N(C)C[C@@H]1C]

To a solution of (2S,5S)—N-(7-methoxy-4-(1-methyl-3-phenyl-1H-pyrazol-4-yl)quinazolin-6-yl)-2,5-dimethylpiperazine-1-carboxamide (Preparation 195, 30.0 mg, 0.064 mmol) in MeOH (2 mL) was added HCHO (5.16 mg, 0.064 mmol) and the solution was stirred at 25° C. for 1 h. NaBH₃CN (6.00 mg, 0.095 mmol) was added and the mixture was stirred at 25° C. for 1 h, then quenched with acetone (1 mL). The solvents were evaporated under reduced pressure. The residue was purified by prep-HPLC (column: Phenomenex Luna C18 75*30 mm*3 um; mobile phase: [water (0.2% FA)-MeCN]; B %: 18%-50%, 8 min) to yield the title compound as a yellow solid (3.90 mg, 11%). LCMS m/z=486 [M+H]⁺. ¹H-NMR (400 MHZ, CDCl₃) δ ppm 9.10 (s, 1H), 8.71 (s, 1H), 7.90 (s, 1H), 7.50-7.42 (m, 3H), 7.41 (s, 1H), 7.23 (s, 3H), 4.24 (d, 1H), 4.13 (s, 3H), 4.11 (s, 3H), 3.80 (d, 1H), 2.98-3.05 (m, 1H), 2.85-2.91 (m, 1H), 2.52 (d, 2H), 2.40 (s, 3H), 1.45 (d, 3H), 1.26 (d, 3H).

### Example 179: (2R,3S)-4-(7-methoxy-4-(1-methyl-3-phenyl-1H-pyrazol-4-yl)quinazolin-6-yl)-2,3-dimethylmorpholine

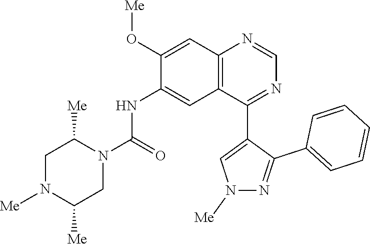 [SMILES: COc1cc2ncnc(-c3cn(C)nc3-c3ccccc3)c2cc1NC(=O)N1C[C@H](C)N(C)C[C@@H]1C]

The title compound was obtained as a yellow solid, 27.3 mg, 57% yield, from 7-methoxy-4-(1-methyl-3-phenyl-1H-pyrazol-4-yl)quinazolin-6-amine (Preparation 190), 4-nitrophenyl chloroformate and (2R,3S)-2,3-dimethylmorpholine (commercial) following a similar procedure to that described in Example 177. LCMS m/z=473 [M+H]⁺. ¹H-NMR (400 MHz, CDCl₃) δ ppm 8.99 (s, 1H), 8.62 (s, 1H), 7.79 (s, 1H), 7.34-7.32 (m, 2H), 7.27 (s, 1H), 7.17 (s, 1H), 7.12-7.10 (m, 3H), 4.01 (s, 3H), 4.00 (s, 3H), 3.92-3.85 (m, 2H), 3.65 (qd, 1H), 3.57-3.51 (m, 2H), 3.23-3.16 (m, 1H), 1.12 (dd, 6H).

### Example 180: (2R,3S)—N-(7-methoxy-4-(1-methyl-3-phenyl-1H-pyrazol-4-yl)quinazolin-6-yl)-2,3,4-trimethylpiperazine-1-carboxamide or (2S,3R)—N-(7-methoxy-4-(1-methyl-3-phenyl-1H-pyrazol-4-yl)quinazolin-6-yl)-2,3,4-trimethylpiperazine-1-carboxamide

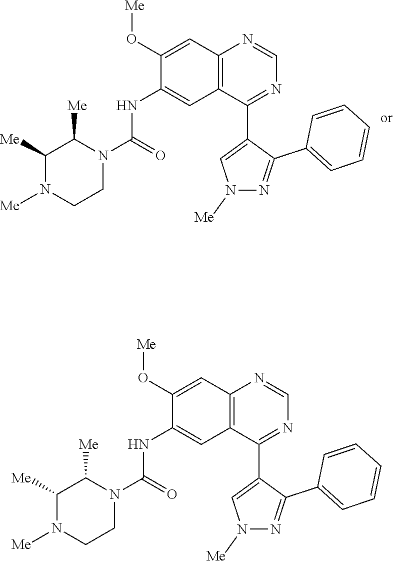 [SMILES: COc1cc2ncnc(-c3cn(C)nc3-c3ccccc3)c2cc1NC(=O)N1CCN(C)[C@@H](C)[C@H]1C ; COc1cc2ncnc(-c3cn(C)nc3-c3ccccc3)c2cc1NC(=O)N1CCN(C)[C@H](C)[C@@H]1C]

The title compound was obtained as a white solid, 7.1 mg, 35% yield, from (2R,3S)—N-(7-methoxy-4-(1-methyl-3-phenyl-1H-pyrazol-4-yl)quinazolin-6-yl)-2,3-dimethylpiperazine-1-carboxamide or (2S,3R)—N-(7-methoxy-4-(1-methyl-3-phenyl-1H-pyrazol-4-yl)quinazolin-6-yl)-2,3-dimethyl piperazine-1-carboxamide (Preparation 196) following a similar procedure to that described in Example 178. LCMS m/z=486 [M+H]⁺. ¹H-NMR (400 MHZ, CDCl₃) δ ppm 8.98 (s, 1H), 8.61 (s, 1H), 7.78 (s, 1H), 7.35-7.31 (m, 2H), 7.19 (s, 1H), 7.17 (s, 1H), 7.13-7.10 (m, 3H), 4.00 (d, 6H), 3.71-3.61 (m, 1H), 3.31 (s, 1H), 2.83 (s, 1H), 2.33-2.16 (m, 6H), 1.18 (d, 3H), 1.07 (d, 3H).

### Example 181: (S)—N-(7-methoxy-4-(1-methyl-3-phenyl-1H-pyrazol-4-yl)quinazolin-6-yl)-2-methyl-4-(methyl-d3) piperazine-1-carboxamide

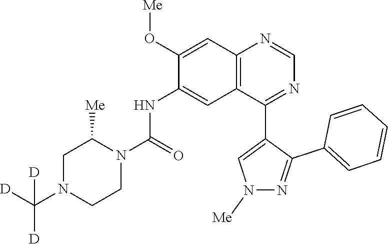 [SMILES: [2H]C([2H])([2H])N1CCN(C(=O)Nc2cc3c(-c4cn(C)nc4-c4ccccc4)ncnc3cc2OC)[C@@H](C)C1]

To a solution of(S)—N-(7-methoxy-4-(1-methyl-3-phenyl-1H-pyrazol-4-yl)quinazolin-6-yl)-2-methylpiperazine-1-carboxamide trifluoroacetate (Preparation 197, 40 mg, 0.070 mmol) and K₂CO₃ (9.67 mg, 0.070 mmol) in DMF (1 mL) was added trideuterio(iodo)methane (20.3 mg, 0.140 mmol) at 0° C. The mixture was stirred at 0° C. for 1 h, then quenched by addition sat. aqueous NH₃ (2 mL, 25%) and the reaction mixture was concentrated in vacuo. The residue was purified by prep-HPLC (Phenomenex Luna C18 75*30 mm*3 um; mobile phase: [water (0.2% FA)-MeCN]; B %: 1%-30%, 8 min) to give the title compound as a yellow solid (24.7 mg, 63% yield, 93% purity) as yellow solid. LCMS m/z=476 [M+H]⁺. ¹H-NMR (400 MHZ, CDCl₃) δ ppm 9.08 (s, 1H), 8.74 (s, 1H), 7.90 (s, 1H), 7.44 (dd, 2H), 7.37 (s, 1H), 7.32 (s, 1H), 7.22 (dd, 3H), 4.24 (d, 2H), 4.11 (d, 6H), 3.82 (d, 1H), 3.36-3.29 (m, 1H), 2.88 (d, 1H), 2.75 (d, 1H), 2.25 (dd, 1H), 2.09-2.02 (m, 1H), 1.39 (d, 3H).

### Example 182: (2S,5R)—N-(7-methoxy-4-(1-methyl-3-phenyl-1H-pyrazol-4-yl)quinazolin-6-yl)-2,4,5-trimethylpiperazine-1-carboxamide

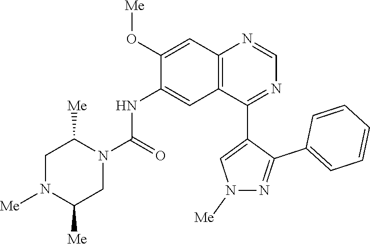 [SMILES: COc1cc2ncnc(-c3cn(C)nc3-c3ccccc3)c2cc1NC(=O)N1C[C@@H](C)N(C)C[C@@H]1C]

The title compound was obtained as a pale yellow solid, 11.9 mg, 9% yield, 92% purity, from (2S,5R)—N-(7-methoxy-4-(1-methyl-3-phenyl-1H-pyrazol-4-yl)quinazolin-6-yl)-2,5-dimethyl piperazine-1-carboxamide (Preparation 198) following a similar procedure to that described in Example 178. LCMS m/z=486 [M+H]⁺. H-NMR (400 MHZ, CDCl₃) δ ppm 9.08 (s, 1H), 8.72 (s, 1H), 7.90 (s, 1H), 7.44 (dd, 2H) 7.36 (s, 1H), 7.32 (s, 1H), 7.22-7.20 (m, 3H), 4.22 (s, 1H), 4.11 (d, 6H), 3.64 (d, 1H), 3.47 (dd, 1H), 2.96 (s, 1H), 2.83 (dd, 1H), 2.38-2.35 (m, 4H), 1.38 (d, 3H), 1.05 (d, 3H).

### Example 183 to 186

The title compounds were prepared from 7-methoxy-4-(1-methyl-3-phenyl-1H-pyrazol-4-yl) quinazolin-6-amine (Preparation 190) and the appropriate amine (RNH₂) using a library protocol as outlined below. To a mixture of 7-methoxy-4-(1-methyl-3-phenyl-1H-pyrazol-4-yl)quinazolin-6-amine (Preparation 190, 69.5 mg, 0.21 mmol) and CDI (178 mg, 0.42 mmol) in DCM (4 mL) was added TEA (0.183 mL, 1.26 mmol) under dry-ice and the mixture shaken for 1 h and then at 60° C. for 16 h. To this was added the appropriate amine (0.32 mmol) under dry-ice and shaking continued for 1 h followed by 30° C. for 16 h. The reaction mixture was filter and evaporated to dryness by Speedvac. The residue was purified by prep-HPLC (Column: Xtimate C18 150*25 mm*5 μm; Mobile Phase: A: NH₄OH/H₂O=0.05% v/v; B: MeCN), to afford the title compounds.

| Example Number | Name/Structure/Amine/Data |
|---|---|
| 183 | (2R,5S)-N-(7-methoxy-4-(1-methyl-3-phenyl-1H-pyrazol-4- |
| | yl)quinazolin-6-yl)-2,5-dimethylmorpholine-4-carboxamide |
| | 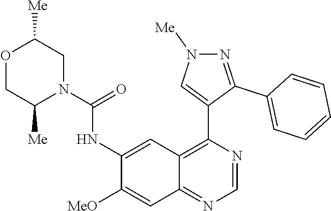 [SMILES: COc1cc2ncnc(-c3cn(C)nc3-c3ccccc3)c2cc1NC(=O)N1C[C@@H](C)OC[C@@H]1C] |
| | Amine: (2R,5S)-2,5-dimethylmorpholine |
| | LCMS: m/z = 473 [M + H]⁺ |
| 184 | N-[7-methoxy-4-(1-methyl-3-phenyl-1H-pyrazol-4-yl)quinazolin-6-yl]- |
| | 2,2-dimethylmorpholine-4-carboxamide |
| | 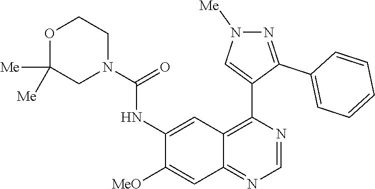 [SMILES: COc1cc2ncnc(-c3cn(C)nc3-c3ccccc3)c2cc1NC(=O)N1CCOC(C)(C)C1] |
| | Amine: 2,2-dimethylmorpholine |
| | LCMS: m/z = 473 [M + H]⁺ |
| 185 | N-[7-methoxy-4-(1-methyl-3-phenyl-1H-pyrazol-4-yl)quinazolin-6-yl]-2- |
| | oxa-5-azabicyclo[4.1.0]heptane-5-carboxamide |
| | 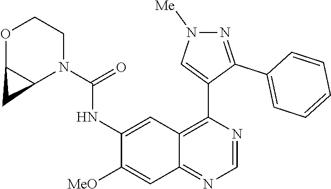 [SMILES: COc1cc2ncnc(-c3cn(C)nc3-c3ccccc3)c2cc1NC(=O)N1CCO[C@@H]2C[C@@H]21] |
| | Amine: (1R,6S)-2-oxa-5-azabicyclo[4.1.0]heptane |
| | LCMS: m/z = 457 [M + H]⁺ |
| 186 | N-[7-methoxy-4-(1-methyl-3-phenyl-1H-pyrazol-4-yl)quinazolin-6-yl]-5- |
| | methyl-2,5-diazabicyclo[4.1.0]heptane-2-carboxamide |
| | 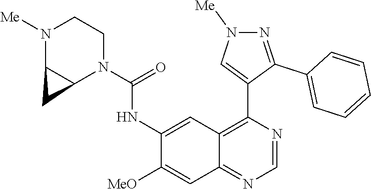 [SMILES: COc1cc2ncnc(-c3cn(C)nc3-c3ccccc3)c2cc1NC(=O)N1CCN(C)[C@@H]2C[C@@H]21] |
| | Amine: (1R,6S)-2-methyl-2,5-diazabicyclo[4.1.0]heptane |
| | LCMS: m/z = 470 [M + H]⁺ |

### Example 187: N-(7-methoxy-4-(1-methyl-3-phenyl-1H-pyrazol-4-yl)quinazolin-6-yl)-1-(trifluoromethyl)-1H-pyrazole-4-carboxamide

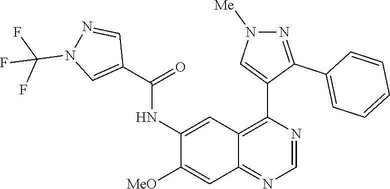 [SMILES: COc1cc2ncnc(-c3cn(C)nc3-c3ccccc3)c2cc1NC(=O)c1cnn(C(F)(F)F)c1]

A mixture of 7-methoxy-4-(1-methyl-3-phenyl-1H-pyrazol-4-yl)quinazolin-6-amine (Preparation 190, 200 mg, 0.604 mmol), 1-(trifluoromethyl)-1H-pyrazole-4-carboxylic acid (108 mg, 603 μmol), T3P® (1.53 g, 2.41 mmol) and pyridine (0.5 mL) in THF was stirred at 50° C. for 2 h. The reaction was extracted with EtOAc (3×60 mL) and washed with water. The combined organics were dried (Na₂SO₄) and evaporated to dryness in vacuo. The residue was purified by prep-HPLC (XBridge Prep OBD C18 Column, 30×150 mm, 5 μm; 32-53% MeCN/H₂O (10 mM NH₄HCO₃+0.1% NH₄OH)) to afford the title compound as a white solid (138 mg, 46%). LCMS: m/z=494 [M+H]⁺; ¹H NMR (300 MHz, DMSO-d₆) δ: 9.67 (s, 1H), 9.20 (s, 1H), 9.07 (s, 1H), 8.49 (s, 1H), 8.45-8.38 (m, 1H), 8.18 (s, 1H), 7.49 (s, 1H), 7.33-7.23 (m, 2H), 7.18 (ddt, 3H), 4.03 (d, 6H).

### Example 188: 1-((dimethylamino)methyl)-N-(4-(3-(2-fluorophenyl)-1-methyl-1H-pyrazol-4-yl)-7-methoxyquinazolin-6-yl)cyclopropane-1-carboxamide

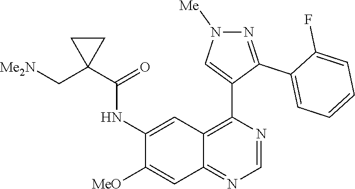 [SMILES: COc1cc2ncnc(-c3cn(C)nc3-c3ccccc3F)c2cc1NC(=O)C1(CN(C)C)CC1]

The title compound was prepared as a white solid (28.5 mg, 21%) from 4-(3-(2-fluorophenyl)-1-methyl-1H-pyrazol-4-yl)-7-methoxyquinazolin-6-amine (Preparation 253) and 1-((dimethylamino)methyl)cyclopropane-1-carboxylic acid using an analogous method to that described for Example 187, except the compound was purified by Prep-HPLC (XBridge Shield RP18 OBD, 30×150 mm, 5 μm; 26-65% MeCN/H₂O (10 mM NH₄HCO₃+0.1% NH₄OH)). LCMS: m/z=380 [M+H]⁺; ¹H NMR (400 MHZ, DMSO-d₆) δ: 12.63 (s, 1H), 8.99 (s, 1H), 8.86 (s, 1H), 8.29 (s, 1H), 7.56 (td, 1H), 7.39 (s, 1H), 7.30 (tdd, 1H), 7.18 (td, 1H), 6.95 (ddd, 1H), 4.09 (s, 3H), 4.04 (s, 3H), 2.36 (s, 6H), 1.15 (q, 2H), 0.66 (q, 2H).

### Example 189-213

The title compounds were prepared from the appropriate amine and appropriate carboxylic acid (RCO₂H) using an analogous method to that described for Example 187. Amine-1:7-methoxy-4-(1-methyl-3-phenyl-1H-pyrazol-4-yl)quinazolin-6-amine (Preparation 190); Amine-2:4-(3-(4-fluorophenyl)-1-methyl-1H-pyrazol-4-yl)-7-methoxyquinazolin-6-amine (Preparation 254): Amine-3:4-(1-(2,2-difluoroethyl)-3-phenyl-1H-pyrazol-4-yl)-7-methoxyquinazolin-6-amine (Preparation 251)

| Example Number | Name/Structure/RCO₂H/Data |
|---|---|
| 189 | 1-((dimethylamino)methyl)-N-(7-methoxy-4-(1-methyl-3-phenyl-1H-pyrazol-4- |
| | yl)quinazolin-6-yl)cyclopropane-1-carboxamide |
| | 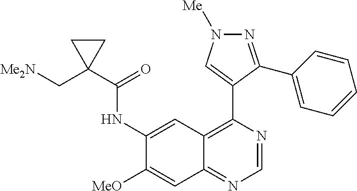 [SMILES: COc1cc2ncnc(-c3cn(C)nc3-c3ccccc3)c2cc1NC(=O)C1(CN(C)C)CC1] |
| | Amine-1; RCO₂H: 1-((dimethylamino)methyl)cyclopropane-1-carboxylic acid |
| | HPLC: XBridge Prep OBD, 30 × 150 mm, 5 μm; 39-59% MeCN/H₂O (10 mM |
| | NH₄HCO₃ + 0.1% NH₃•H₂O) |
| | White solid (14.6 mg, 11%). LCMS: m/z = 457 [M + H]⁺ ; ¹H NMR (400 MHz, |
| | DMSO-d₆) δ: 12.63 (s, 1H), 9.01 (s, 1H), 8.85 (s, 1H), 8.15 (s, 1H), 7.42 (s, 1H), |
| | 7.30-7.22 (m, 2H), 7.22-7.14 (m, 3H), 4.10 (s, 3H), 4.01 (s, 3H), 3.35 (s, 2H), 2.35 |
| | (s, 6H), 1.11 (q, 2H), 0.63 (q, 2H). |
| 190 | 3-chloro-N-(7-methoxy-4-(1-methyl-3-phenyl-1H-pyrazol-4-yl)quinazolin-6- |
| | yl)isonicotinamide |
| | 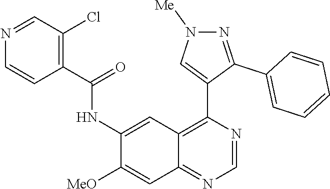 [SMILES: COc1cc2ncnc(-c3cn(C)nc3-c3ccccc3)c2cc1NC(=O)c1ccncc1Cl] |
| | Amine-1; RCO₂H: 3-chloroisonicotinic acid |
| | HPLC: XBridge Prep OBD, 30 × 150 mm, 5 μm; 19-49% MeCN/H₂O (10 mM |
| | NH₄HCO₃ + 0.1% NH₄OH) |
| | White solid (38.5 mg, 27%). LCMS: m/z = 471 [M + H]⁺; ¹H NMR (300 MHz, |
| | DMSO-d₆) δ: 10.31 (s, 1H), 9.10 (s, 1H), 8.74 (d, 2H), 8.64 (d, 1H), 8.24 (s, 1H), |
| | 7.57-7.45 (m, 2H), 7.33-7.23 (m, 5H), 4.04 (d, 6H). |
| 191 | N-(7-methoxy-4-(1-methyl-3-phenyl-1H-pyrazol-4-yl)quinazolin-6-yl)-3- |
| | methylisonicotinamide |
| | 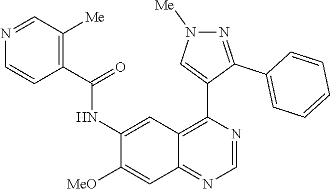 [SMILES: COc1cc2ncnc(-c3cn(C)nc3-c3ccccc3)c2cc1NC(=O)c1ccncc1C] |
| | Amine-1; RCO₂H: 3-methylpyridine-4-carboxylic acid |
| | HPLC: XBridge Prep OBD, 30 × 150 mm, 5 μm; 31-56% MeCN/H₂O (10 mM |
| | NH₄HCO₃ + 0.1% NH₄OH) |
| | White solid (57.6 mg, 60%). LCMS: m/z = 451 [M + H]⁺; ¹H NMR (300 MHz, |
| | DMSO-d₆) δ: 9.94 (s, 1H), 9.08 (s, 1H), 8.58 (s, 1H), 8.55-8.41 (m, 2H), 8.22 (s, |
| | 1H), 7.45 (s, 1H), 7.34 (d, 1H), 7.30-7.20 (m, 2H), 7.24-7.14 (m, 3H), 4.01 (d, 6H), |
| | 2.28 (s, 3H). |
| 192 | N-(7-methoxy-4-(1-methyl-3-phenyl-1H-pyrazol-4-yl)quinazolin-6-yl)pyrimidine- |
| | 2-carboxamide |
| | 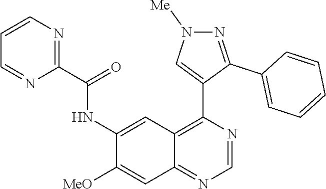 [SMILES: COc1cc2ncnc(-c3cn(C)nc3-c3ccccc3)c2cc1NC(=O)c1ncccn1] |
| | Amine-1; RCO₂H: pyrimidine-2-carboxylic acid |
| | HPLC: XBridge Prep OBD, 30 × 150 mm, 5 μm; 17-47% MeCN/H₂O (10 mM |
| | NH₄HCO₃ + 0.1% NH₄OH) |
| | White solid (8.1 mg, 12%). LCMS: m/z = 438 [M + H]⁺; ¹H NMR (300 MHz, |
| | DMSO-d₆) δ: 10.63 (s, 1H), 9.09-9.01 (m, 4H), 8.24 (s, 1H), 7.77 (t, 1H), 7.54 (s, |
| | 1H), 7.35-7.25 (m, 2H), 7.24-7.12 (m, 3H), 4.15 (s, 3H), 4.04 (s, 3H). |
| 193 | N-(7-methoxy-4-(1-methyl-3-phenyl-1H-pyrazol-4-yl)quinazolin-6-yl)pyrimidine- |
| | 5-carboxamide |
| | 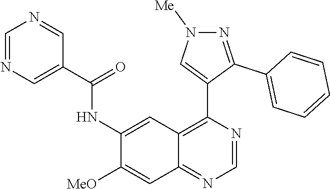 [SMILES: COc1cc2ncnc(-c3cn(C)nc3-c3ccccc3)c2cc1NC(=O)c1cncnc1] |
| | Amine-1; RCO₂H: pyrimidine-5-carboxylic acid |
| | HPLC: XBridge Prep OBD, 30 × 150 mm, 5 μm; 29-45% MeCN/H₂O (10 mM |
| | NH₄HCO₃ + 0.1% NH₄OH) |
| | White solid (28.1 mg, 27%). LCMS: m/z = 438 [M + H]⁺; ¹H NMR (300 MHz, |
| | DMSO-d₆) δ: 10.22 (s, 1H), 9.36 (s, 1H), 9.19 (s, 2H), 9.10 (s, 1H), 8.58 (s, 1H), |
| | 8.21 (s, 1H), 7.52 (s, 1H), 7.35-7.26 (m, 2H), 7.24-7.16 (m, 3H), 4.08 (s, 3H), 4.03 |
| | (s, 3H). |
| 194 | N-(7-methoxy-4-(1-methyl-3-phenyl-1H-pyrazol-4-yl)quinazolin-6-yl)-2- |
| | methylisonicotinamide |
| | 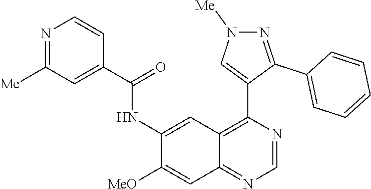 [SMILES: COc1cc2ncnc(-c3cn(C)nc3-c3ccccc3)c2cc1NC(=O)c1ccnc(C)c1] |
| | Amine-1; RCO₂H: 2-methylpyridine-4-carboxylic acid |
| | HPLC: XBridge Prep OBD, 30 × 150 mm, 5 μm; 28-38% MeCN/H₂O (10 mM |
| | NH₄HCO₃ + 0.1% NH₃•H₂O) |
| | White solid (50.1 mg, 46%). LCMS: m/z = 451 [M + H]⁺; ¹H NMR (400 MHz, |
| | DMSO-d₆) δ: 9.94 (s, 1H), 9.10 (s, 1H), 8.66 (d, 1H), 8.53 (s, 1H), 8.22 (s, 1H), |
| | 7.74 (s, 1H), 7.65 (d, 1H), 7.52 (s, 1H), 7.30 (ddt, 2H), 7.24-7.16 (m, 3H), 4.05 (d, |
| | 3H), 4.01 (s, 3H), 2.59 (s, 3H). |
| 195 | N-(7-methoxy-4-(1-methyl-3-phenyl-1H-pyrazol-4-yl)quinazolin-6-yl)-2- |
| | methylnicotinamide |
| | 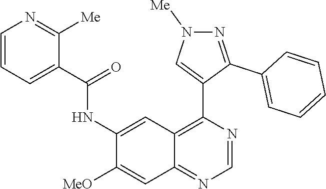 [SMILES: COc1cc2ncnc(-c3cn(C)nc3-c3ccccc3)c2cc1NC(=O)c1cccnc1C] |
| | Amine-1; RCO₂H: 2-methylnicotinic acid |
| | HPLC: YMC-Actus Triart 30 × 150 mm, 5 μm; 20-50% MeCN/H₂O (10 mM |
| | NH₄HCO₃ + 0.1% NH₄OH) |
| | Off-white solid (32 mg, 24%). LCMS: m/z = 451 [M + H]⁺; ¹H NMR (300 MHz, |
| | DMSO-d₆) δ: 9.91 (s, 1H), 9.10 (s, 1H), 8.63 (s, 2H), 8.24 (s, 1H), 7.78 (d, 1H), |
| | 7.47 (s, 1H), 7.37-7.16 (m, 6H), 4.03 (d, 6H), 3.2 (s, 3H). |
| 196 | 5-fluoro-N-(7-methoxy-4-(1-methyl-3-phenyl-1H-pyrazol-4-yl)quinazolin-6-yl)-6- |
| | methylnicotinamide |
| | 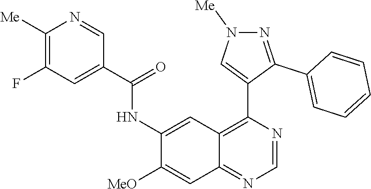 [SMILES: COc1cc2ncnc(-c3cn(C)nc3-c3ccccc3)c2cc1NC(=O)c1cnc(C)c(F)c1] |
| | Amine-1; RCO₂H: 5-fluoro-6-methylpyridine-3-carboxylic acid |
| | HPLC: YMC-Actus Triart 30 × 150 mm, 5 μm; 27-57% MeCN/H₂O (10 mM |
| | NH₄HCO₃ + 0.1% NH₄OH) |
| | White solid (46.6 mg, 66%). LCMS: m/z = 469 [M + H]⁺; ¹H NMR (400 MHz, |
| | DMSO-d₆) δ: 9.97 (s, 1H), 9.10 (s, 1H), 8.83 (t, 1H), 8.51 (s, 1H), 8.22 (s, 1H), |
| | 8.09 (dd, 1H), 7.52 (s, 1H), 7.31 (ddt, 2H), 7.21 (ddt, 3H), 4.08-4.03 (m, 6H), 2.54 |
| | (d, 3H). |
| 197 | N-(7-methoxy-4-(1-methyl-3-phenyl-1H-pyrazol-4-yl)quinazolin-6-yl)-2- |
| | methylbenzamide |
| | 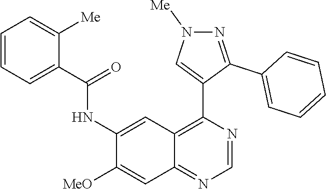 [SMILES: COc1cc2ncnc(-c3cn(C)nc3-c3ccccc3)c2cc1NC(=O)c1ccccc1C] |
| | Amine-1; RCO₂H: 2-methylbenzoic acid |
| | HPLC: YMC-Actus Triart 30 × 150 mm, 5 μm; 31-64% MeCN/H₂O (10 mM |
| | NH₄HCO₃ + 0.1% NH₄OH) |
| | White solid (40.9 mg, 37%). LCMS: m/z = 450 [M + H]⁺; ¹H NMR (400 MHz, |
| | DMSO-d₆) δ: 9.50 (s, 1H), 9.08 (s, 1H), 8.60 (s, 1H), 8.23 (s, 1H), 7.46-7.37 (m, |
| | 3H), 7.29-7.19 (m, 7H), 4.03 (s, 1H), 4.02 (s, 1H), 2.07 (s, 1H). |
| 198 | 2-fluoro-N-(7-methoxy-4-(1-methyl-3-phenyl-1H-pyrazol-4-yl)quinazolin-6- |
| | yl)benzamide |
| | 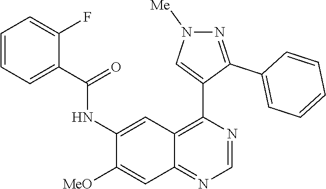 [SMILES: COc1cc2ncnc(-c3cn(C)nc3-c3ccccc3)c2cc1NC(=O)c1ccccc1F] |
| | Amine-1; RCO₂H: 2-fluorobenzoic acid |
| | Product collected by filtration. |
| | White solid (20.4 mg, 18%). LCMS: m/z = 454 [M + H]⁺; ¹H NMR (400 MHz, |
| | DMSO-d₆) δ: 9.72 (d, 1H), 9.08 (s, 1H), 8.86 (s, 1H), 8.23 (s, 1H), 7.84 (t, 1H), |
| | 7.69-7.59 (m, 1H), 7.51 (s, 1H), 7.43-7.33 (m, 2H), 7.30 (dt, 2H), 7.19 (p, 3H), |
| | 4.10 (s, 3H), 4.05 (s, 3H). |
| 199 | 2-chloro-3-fluoro-N-(7-methoxy-4-(1-methyl-3-phenyl-1H-pyrazol-4- |
| | yl)quinazolin-6-yl)benzamide |
| | 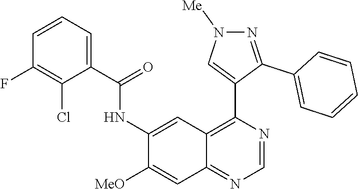 [SMILES: COc1cc2ncnc(-c3cn(C)nc3-c3ccccc3)c2cc1NC(=O)c1cccc(F)c1Cl] |
| | Amine-1; RCO₂H: 2-chloro-3-fluorobenzoic acid |
| | HPLC: XBridge Shield RP18 OBD, 30 × 150 mm, 5 μm; 35-55% MeCN/H₂O (10 |
| | mM NH₄HCO₃ + 0.1% NH₄OH) |
| | White solid (69.2 mg, 59%). LCMS: m/z = 488 [M + H]⁺; ¹H NMR (300 MHz, |
| | DMSO-d₆) δ: 10.11 (s, 1H), 9.10 (s, 1H), 8.71 (s, 1H), 8.24 (s, 1H), 7.49 (d, 3H), |
| | 7.40-7.16 (m, 6H), 4.04 (d, 6H). |
| 200 | 3-fluoro-N-(7-methoxy-4-(1-methyl-3-phenyl-1H-pyrazol-4-yl)quinazolin-6-yl)-2- |
| | methylbenzamide |
| | 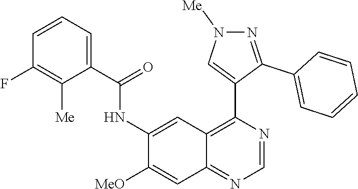 [SMILES: COc1cc2ncnc(-c3cn(C)nc3-c3ccccc3)c2cc1NC(=O)c1cccc(F)c1C] |
| | Amine-1; RCO₂H: 3-fluoro-2-methylbenzoic acid |
| | HPLC: XBridge Shield RP18 OBD, 30 × 150 mm, 5 μm; 35-55% MeCN/H₂O (10 |
| | mM NH₄HCO₃ + 0.1% NH₄OH) |
| | Off-white solid (49.4 mg, 44%). LCMS: m/z = 468 [M + H]⁺; ¹H NMR (400 MHz, |
| | DMSO-d₆) δ: 9.71 (s, 1H), 9.10 (s, 1H), 8.60 (s, 1H), 8.24 (s, 1H), 7.46 (s, 1H), |
| | 7.35-7.19 (m, 8H), 4.03 (d, 6H), 2.23 (d, 3H). |
| 201 | 2-fluoro-N-(7-methoxy-4-(1-methyl-3-phenyl-1H-pyrazol-4-yl)quinazolin-6-yl)-6- |
| | methylbenzamide |
| | 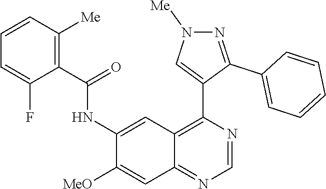 [SMILES: COc1cc2ncnc(-c3cn(C)nc3-c3ccccc3)c2cc1NC(=O)c1c(C)cccc1F] |
| | Amine-1; RCO₂H: 2-fluoro-6-methylbenzoic acid |
| | HPLC: YMC-Actus Triart C19 ExRS, 30 × 150 mm, 5 μm; 29-62% MeCN/H₂O |
| | (10 mM NH₄HCO₃ + 0.1% NH₄OH) |
| | White solid (17.3 mg, 15%). LCMS: m/z = 468 [M + H]⁺; ¹H NMR (300 MHz, |
| | DMSO-d₆) δ: 10.06 (s, 1H), 9.11 (s, 1H), 8.70 (s, 1H), 8.25 (s, 1H), 7.46 (s, 1H), |
| | 7.38 (td, 1H), 7.28 (dtd, 2H), 7.21 (dt, 3H), 7.15-7.05 (m, 2H), 4.03 (d, 6H), 2.25 |
| | (s, 3H). |
| 202 | 1-ethyl-N-(7-methoxy-4-(1-methyl-3-phenyl-1H-pyrazol-4-yl)quinazolin-6-yl)-1H- |
| | pyrazole-3-carboxamide |
| | 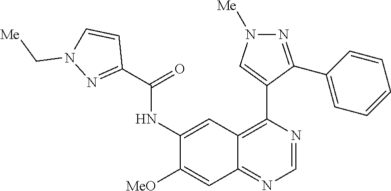 [SMILES: CCn1ccc(C(=O)Nc2cc3c(-c4cn(C)nc4-c4ccccc4)ncnc3cc2OC)n1] |
| | Amine-1; RCO₂H: 1-ethyl-1H-pyrazole-3-carboxylic acid |
| | HPLC: YMC-Actus Triart C19 ExRS, 30 × 150 mm, 5 μm; 27-60% MeCN/H₂O |
| | (10 mM NH₄HCO₃ + 0.1% NH₄OH) |
| | White solid (70.7 mg, 52%). LCMS: m/z = 454 [M + H]⁺; ¹H NMR (400 MHz, |
| | DMSO-d₆) δ: 9.48 (s, 1H), 9.05 (s, 1H), 8.95 (s, 1H), 8.22 (s, 1H), 7.94 (d, 1H), |
| | 7.52 (s, 1H), 7.36-7.27 (m, 2H), 7.19 (qd, 3H), 6.78 (d, 1H), 4.26 (q, 2H), 4.14 (s, |
| | 3H), 4.05 (s, 3H), 1.44 (t, 3H). |
| 203 | N-(7-methoxy-4-(1-methyl-3-phenyl-1H-pyrazol-4-yl)quinazolin-6-yl)-1-(2,2,2- |
| | trifluoroethyl)-1H-pyrazole-4-carboxamide |
| | 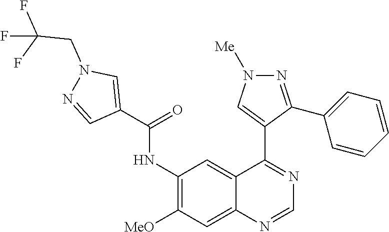 [SMILES: COc1cc2ncnc(-c3cn(C)nc3-c3ccccc3)c2cc1NC(=O)c1cnn(CC(F)(F)F)c1] |
| | Amine-1; RCO₂H: 1-(2,2,2-trifluoroethyl)-1H-pyrazole-4-carboxylic acid |
| | HPLC: XBridge Prep OBD C18, 30 × 150 mm, 5 μm; 35-55% MeCN/H₂O |
| | (10 mM NH₄HCO₃ + 0.1% NH₄OH) |
| | White solid (66.1 mg, 86%). LCMS: m/z = 508 [M + H]⁺; ¹H NMR (300 MHz, |
| | DMSO-d₆) δ: 9.44 (s, 1H), 9.05 (s, 1H), 8.50 (d, 2H), 8.16 (d, 2H), 7.48 (s, 1H), |
| | 7.33-7.24 (m, 2H), 7.18 (dt, 3H), 5.20 (d, 2H), 4.03 (d, 6H). |
| 204 | N-(7-methoxy-4-(1-methyl-3-phenyl-1H-pyrazol-4-yl)quinazolin-6-yl)-1- |
| | (trifluoromethyl)-1H-pyrazole-5-carboxamide |
| | 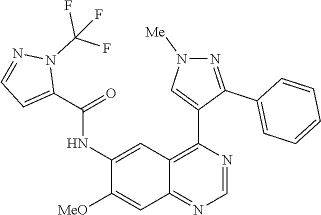 [SMILES: COc1cc2ncnc(-c3cn(C)nc3-c3ccccc3)c2cc1NC(=O)c1ccnn1C(F)(F)F] |
| | Amine-1; RCO₂H: 1-(trifluoromethyl)-1H-pyrazole-5-carboxylic acid (Preparation |
| | 82) |
| | HPLC: XBridge Shield RP18 OBD, 30 × 150 mm, 5 μm; 22-58% MeCN/H₂O |
| | (10 mM NH₄HCO₃ + 0.1% NH₄OH) |
| | White solid (45.3 mg, 33%). LCMS: m/z = 494 [M + H]⁺; ¹H NMR (400 MHz, |
| | DMSO-d₆) δ: 10.30 (s, 1H), 9.12 (s 1H), 8.48-8.43 (m, 1H), 8.24 (s, 1H), 8.01 (d, |
| | 1H), 7.50 (s, 1H), 7.31-7.22 (m, 2H), 7.20 (qd, 3H), 7.10 (s, 1H), 4.04 (d, 6H) |
| 205 | 1-isopropyl-N-(7-methoxy-4-(1-methyl-3-phenyl-1H-pyrazol-4-yl)quinazolin-6- |
| | yl)-1H-pyrazole-3-carboxamide |
| | 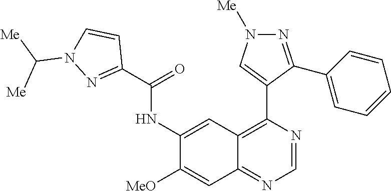 [SMILES: COc1cc2ncnc(-c3cn(C)nc3-c3ccccc3)c2cc1NC(=O)c1ccn(C(C)C)n1] |
| | Amine-1: RCO₂H: 1-isopropyl-1H-pyrazole-3-carboxylic acid |
| | HPLC: YMC-Actus Triart C19 ExRS, 30 × 150 mm, 5 μm; 40-70% MeCN/H₂O |
| | (10 mM NH₄HCO₃ + 0.1% NH₄OH) |
| | White solid (87.5 mg, 62%). LCMS: m/z = 468 [M + H]⁺; ¹H NMR (300 MHz, |
| | DMSO-d₆) δ: 9.50 (s, 1H), 9.04 (s, 1H), 8.92 (s, 1H), 8.21 (s, 1H), 7.97 (d, 1H), |
| | 7.50 (s, 1H), 7.33-7.24 (m, 2H), 7.17 (dd, 3H), 6.77 (d, 1H), 4.61 (p, 1H), 4.13 (s, |
| | 3H), 4.03 (s, 3H), 1.46 (d, 6H). |
| 206 | N-(7-methoxy-4-(1-methyl-3-phenyl-1H-pyrazol-4-yl)quinazolin-6-yl)-2- |
| | methylthiazole-5-carboxamide |
| | 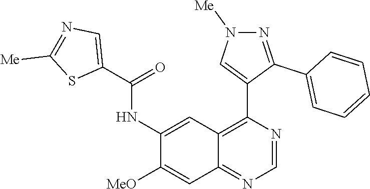 [SMILES: COc1cc2ncnc(-c3cn(C)nc3-c3ccccc3)c2cc1NC(=O)c1cnc(C)s1] |
| | Amine-1; RCO₂H: 2-methyl-1,3-thiazole-5-carboxylic acid |
| | Product collected by filtration. White solid (20.1 mg, 15%). LCMS: m/z = 457 |
| | [M + H]⁺; ¹H NMR (400 MHz, DMSO-d₆) δ: 9.83 (s, 1H), 9.09 (s, 1H), 8.46 (s, 2H), |
| | 8.22 (s, 1H), 7.52 (s, 1H), 7.30 (dd, 2H), 7.21 (q, 3H), 4.08 (s, 6H), 2.71 (s, 3H). |
| 207 | N-(7-methoxy-4-(1-methyl-3-phenyl-1H-pyrazol-4-yl)quinazolin-6-yl)-4- |
| | methylthiazole-5-carboxamide |
| | 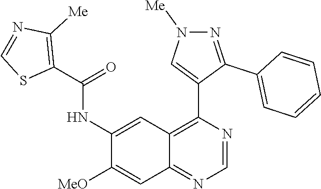 [SMILES: COc1cc2ncnc(-c3cn(C)nc3-c3ccccc3)c2cc1NC(=O)c1scnc1C] |
| | Amine-1; RCO₂H: 4-methyl-1,3-thiazole-5-carboxylic acid |
| | Product collected by filtration. White solid (72 mg, 52%). LCMS: m/z = 457 |
| | [M + H]⁺; ¹H NMR (400 MHz, DMSO-d₆) δ: 9.52 (s, 1H), 9.15 (s, 1H), 9.09 (s, 1H), |
| | 8.56 (s, 1H), 8.22 (s, 1H), 7.51 (s, 1H), 7.33-7.25 (m, 2H), 7.21 (dp, 3H), 4.06 (d, |
| | 6H), 2.66 (s, 3H), 1.55-1.45 (m, 1H), 1.00-0.91 (m, 1H). |
| 208 | 1-isopropyl-N-(7-methoxy-4-(1-methyl-3-phenyl-1H-pyrazol-4-yl)quinazolin-6- |
| | yl)-1H-pyazole-4-carboxamide |
| | 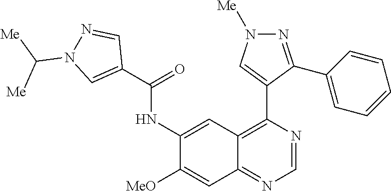 [SMILES: COc1cc2ncnc(-c3cn(C)nc3-c3ccccc3)c2cc1NC(=O)c1cnn(C(C)C)c1] |
| | Amine-1; RCO₂H: 1-isopropyl-1H-pyrazole-4-carboxylic acid |
| | HPLC: YMC-Actus Triart C19 ExRS, 30 × 150 mm, 5 μm; 25-55% MeCN/H₂O |
| | (10 mM NH₄HCO₃ + 0.1% NH₄OH) |
| | White solid (38.3 mg, 27%). LCMS: m/z = 468 [M + H]⁺; ¹H NMR (300 MHz, |
| | DMSO-d₆) δ: 9.24 (s, 1H), 9.06 (s, 1H), 8.56 (s, 1H), 8.47 (s, 1H), 8.21 (s, 1H), |
| | 8.02 (s, 1H), 7.50 (s, 1H), 7.31 (dd, 2H), 7.25-7.16 (m, 3H), 4.61-4.50 (m, 1H), |
| | 4.06 (d, 6H), 1.45 (d, 6H). |
| 209 | 2-cyclopropyl-N-(7-methoxy-4-(1-methyl-3-phenyl-1H-pyrazol-4-yl)quinazolin-6- |
| | yl)thiazole-5-carboxamide |
| | 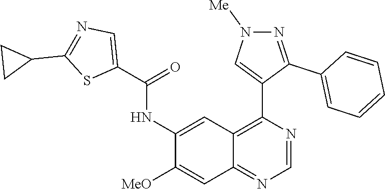 [SMILES: COc1cc2ncnc(-c3cn(C)nc3-c3ccccc3)c2cc1NC(=O)c1cnc(C2CC2)s1] |
| | Amine-1; RCO₂H: 2-cyclopropyl-1,3-thiazole-5-carboxylic acid |
| | HPLC: YMC-Actus Triart C19 ExRS, 30 × 150 mm, 5 μm; 31-58% MeCN/H₂O |
| | (10 mM NH₄HCO₃ + 0.1% NH₄OH) |
| | White solid (21.1 mg, 14%). LCMS: m/z = 483 [M + H]⁺; ¹H NMR (400 MHz, |
| | DMSO-d₆) δ: 9.81 (s, 1H), 9.08 (s, 1H), 8.42 (s, 1H), 8.38 (s, 1H), 8.21 (s, 1H), |
| | 7.51 (s, 1H), 7.31-7.28 (m, 2H), 7.31-7.19 (m, 3H), 4.07 (s, 3H), 4.02 (s, 3H), 2.49- |
| | 2.45 (m, 1H), 1.21-1.16 (m, 2H), 1.06-1.02 (m, 2H). |
| 210 | (R)-N-(7-methoxy-4-(1-methyl-3-phenyl-1H-pyrazol-4-yl)quinazolin-6-yl)-1- |
| | methylpyrrolidine-3-carboxamide |
| | 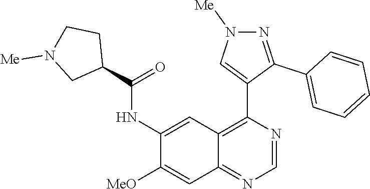 [SMILES: COc1cc2ncnc(-c3cn(C)nc3-c3ccccc3)c2cc1NC(=O)[C@@H]1CCN(C)C1] |
| | Amine-1; RCO₂H: (3R)-1-methylpyrrolidine-3-carboxylic acid |
| | HPLC: XSelect CSH C18 OBD, 30 × 150 mm, 5 μm; 20-35% MeCN/H₂O |
| | (10 mM NH₄HCO₃ + 0.1% NH₄OH) |
| | White solid (44.9 mg, 48%). LCMS: m/z = 443 [M + H]⁺; ¹H NMR (300 MHz, |
| | DMSO-d₆) δ: 9.89 (s, 1H), 9.03 (s, 1H), 8.71 (s, 1H), 8.19 (s, 1H), 7.42 (s, 1H), |
| | 7.31-7.20 (m, 2H), 7.19 (dt, 3H), 4.05 (d, 6H), 3.08 (s, 1H), 2.61 (dt, 2H), 2.47- |
| | 2.34 (m, 2H), 2.32 (s, 3H), 2.10-1.97 (m, 2H). |
| 211 | 1-((dimethylamino)methyl)-N-(4-(3-(4-fluorophenyl)-1-methyl-1H-pyrazol-4-yl)- |
| | 7-methoxyquinazolin-6-yl)cyclopropane-1-carboxamide |
| | 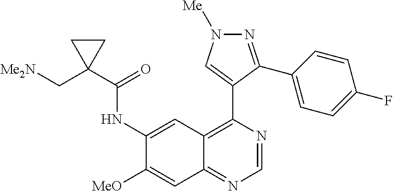 [SMILES: COc1cc2ncnc(-c3cn(C)nc3-c3ccc(F)cc3)c2cc1NC(=O)C1(CN(C)C)CC1] |
| | Amine-2; Acid: 1-((dimethylamino)methyl)cyclopropane-1-carboxylic acid |
| | HPLC: XBridge Shield RP18 OBD Column, 30 × 150 mm, 5 μm; 30-60% |
| | MeCN/H₂O (10 mM NH₄CO₃ + 0.1% NH₄OH) |
| | White solid (31.1 mg, 23%); LCMS: m/z = 475 [M + H]⁺; ¹H NMR (300 MHz, |
| | DMSO-d₆) δ: 12.62 (s, 1H), 8.99 (s, 1H), 8.82 (s, 1H), 8.15 (s, 1H), 7.41 (s, 1H), |
| | 7.34-7.14 (m, 2H), 7.08-6.87 (m, 2H), 4.08 (s, 3H), 3.99 (s, 3H), 2.48 (d, 2H), 2.33 |
| | (s, 6H), 1.09 (q, 2H), 0.62 (q, 2H). |
| 212 | 3,3-difluoro-N-(4-(3-(4-fluorophenyl)-1-methyl-1H-pyrazol-4-yl)-7- |
| | methoxyquinazolin-6-yl)cyclobutane-1-carboxamide |
| | 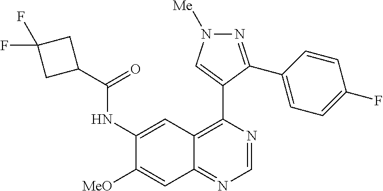 [SMILES: COc1cc2ncnc(-c3cn(C)nc3-c3ccc(F)cc3)c2cc1NC(=O)C1CC(F)(F)C1] |
| | Amine-2; Acid: 3,3-difluorocyclobutane-1-carboxylic acid |
| | HPLC: XBridge Shield RP18 OBD Column, 30 × 150 mm, 5 μm; 18-54% |
| | MeCN/H₂O (10 mM NH₄HCO₃ + 0.1% NH₄OH) |
| | White solid (63.7 mg); LCMS: m/z = 468 [M + H]⁺; ¹H NMR (400 MHz, DMSO-d₆) |
| | δ: 9.65 (s, 1H), 9.05 (s, 1H), 8.74 (s, 1H), 8.21 (s, 1H), 7.44 (s, 1H), 7.35-7.25 (m, |
| | 2H), 7.08-6.98 (m, 2H), 4.05 (d, 6H), 3.52 (s, 1H), 2.74 (dt, 4H). |
| 213 | N-(4-(1-(2,2-difluoroethyl)-3-phenyl-1H-pyrazol-4-yl)-7-methoxyquinazolin-6- |
| | yl)thiazole-5-carboxamide |
| | 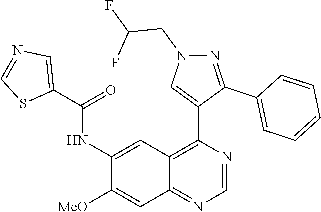 [SMILES: COc1cc2ncnc(-c3cn(CC(F)F)nc3-c3ccccc3)c2cc1NC(=O)c1cncs1] |
| | Amine-3; Acid: thiazole-5-carboxylic acid |
| | HPLC: XBridge Shield RP18 OBD Column, 30 × 150 mm, 5 μm; 9-22% |
| | MeCN/H₂O (10 mM NH₄HCO₃ + 0.1% NH₄OH) |
| | Off-white solid (76 mg, 39%); LCMS: m/z = 493 [M + H]⁺; ¹H NMR (300 MHz, |
| | DMSO-d₆) δ: 9.99 (s, 1H), 9.32 (s, 1H), 9.12 (s, 1H), 8.72 (s, 1H), 8.34 (d, 2H), |
| | 7.54 (s, 1H), 7.31 (dd, 2H), 7.22 (dd, 3H), 6.88-6.26 (m, 1H), 4.83 (td, 2H), 4.08 (s, |
| | 3H). |

### Example 214-228

The title compounds were prepared using a 1-step library protocol as outlined below.

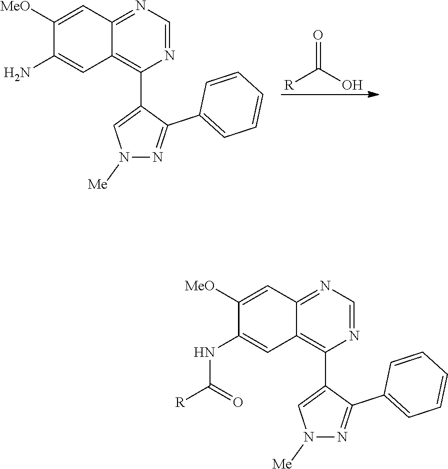

Pyridine (1 mL) and T₃P® (1 mL) were added to a mixture of 7-methoxy-4-(1-methyl-3-phenyl-1H-pyrazol-4-yl)quinazolin-6-amine (Preparation 190, 66.3 mg, 0.20 mmol) and the appropriate carboxylic acid (RCO₂H, 0.20 mmol) in THF (1 mL) and the mixture shaken at 50° C. for 5 h. The reaction mixture was evaporated to dryness by Speedvac. The residues were purified by prep-HPLC (Xtimate C18 25×150 mm, 5 μm; MeCN/(0.05% v/v NH₄OH/H₂O); solvent gradient optimized for each compound) to give the title compounds.

| Example | |
|---|---|
| Number | Name/Structure/RCO₂H/Data |
| 214 | N-(7-methoxy-4-(1-methyl-3-phenyl-1H-pyrazol-4-yl)quinazolin-6-yl)-1- |
| | (methoxymethyl)cyclobutane-1-carboxamide |
| | 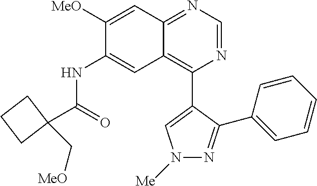 [SMILES: COCC1(C(=O)Nc2cc3c(-c4cn(C)nc4-c4ccccc4)ncnc3cc2OC)CCC1] |
| | RCO₂H: 1-(methoxymethyl)cyclobutane-1-carboxylic acid |
| | LCMS: m/z = 458 [M + H]⁺ |
| 215 | 3-methoxy-N-(7-methoxy-4-(1-methyl-3-phenyl-1H-pyrazol-4-yl)quinazolin-6- |
| | yl)cyclobutane-1-carboxamide |
| | 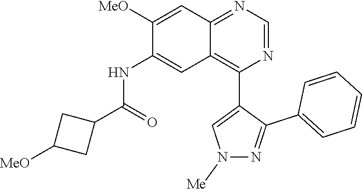 [SMILES: COc1cc2ncnc(-c3cn(C)nc3-c3ccccc3)c2cc1NC(=O)C1CC(OC)C1] |
| | RCO₂H: 3-methoxycyclobutane-1-carboxylic acid |
| | LCMS: m/z = 444 [M + H]⁺ |
| 216 | N-(7-methoxy-4-(1-methyl-3-phenyl-1H-pyrazol-4-yl)quinazolin-6-yl)-1-(pyridin- |
| | 3-yl)cyclopropane-1-carboxamide |
| | 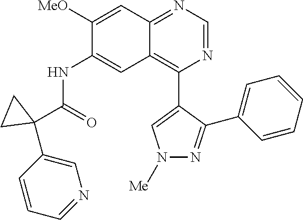 [SMILES: COc1cc2ncnc(-c3cn(C)nc3-c3ccccc3)c2cc1NC(=O)C1(c2cccnc2)CC1] |
| | RCO₂H: 1-(pyridin-3-yl)cyclopropane-1-carboxylic acid |
| | LCMS: m/z = 477 [M + H]⁺ |
| 217 | 1-isopropyl-N-(7-methoxy-4-(1-methyl-3-phenyl-1H-pyrazol-4-yl)quinazolin-6- |
| | yl)-3-methyl-1H-pyrazole-4-carboxamide |
| | 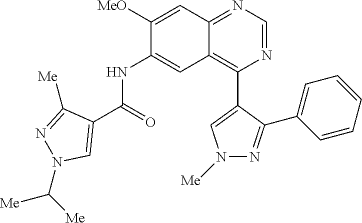 [SMILES: COc1cc2ncnc(-c3cn(C)nc3-c3ccccc3)c2cc1NC(=O)c1cn(C(C)C)nc1C] |
| | RCO₂H: 1-isopropyl-3-methyl-1H-pyrazole-4-carboxylic acid |
| | LCMS: m/z = 482 [M + H]⁺ |
| 218 | 1-isopropyl-N-(7-methoxy-4-(1-methyl-3-phenyl-1H-pyrazol-4-yl)quinazolin-6- |
| | yl)-1H-pyrazole-5-carboxamide |
| | 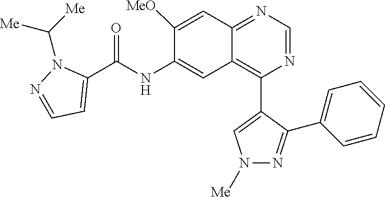 [SMILES: COc1cc2ncnc(-c3cn(C)nc3-c3ccccc3)c2cc1NC(=O)c1ccnn1C(C)C] |
| | RCO₂H: 1-isopropyl-1H-pyrazole-5-carboxylic acid |
| | LCMS: m/z = 468 [M + H]⁺ |
| 219 | 1-ethyl-N-(7-methoxy-4-(1-methyl-3-phenyl-1H-pyrazol-4-yl)quinazolin-6-yl)-1H- |
| | pyrazole-4-carboxamide |
| | 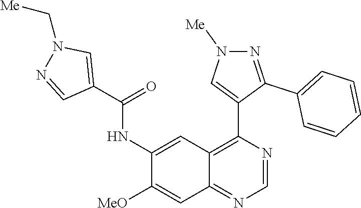 [SMILES: CCn1cc(C(=O)Nc2cc3c(-c4cn(C)nc4-c4ccccc4)ncnc3cc2OC)cn1] |
| | RCO₂H: 1-ethyl-1H-pyrazole-5-carboxylic acid |
| | LCMS: m/z = 454 [M + H]⁺ |
| 220 | 2-(dimethylamino)-N-(7-methoxy-4-(1-methyl-3-phenyl-1H-pyrazol-4- |
| | yl)quinazolin-6-yl)propanamide |
| | 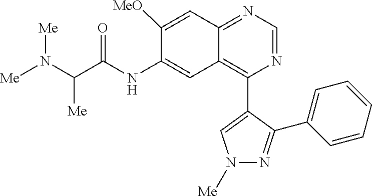 [SMILES: COc1cc2ncnc(-c3cn(C)nc3-c3ccccc3)c2cc1NC(=O)C(C)N(C)C] |
| | RCO₂H: dimethylalanine |
| | LCMS: m/z = 431 [M + H]⁺ |
| 221 | 2-fluoro-N-(7-methoxy-4-(1-methyl-3-phenyl-1H-pyrazol-4-yl)quinazolin-6- |
| | yl)propanamide |
| | 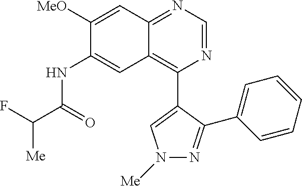 [SMILES: COc1cc2ncnc(-c3cn(C)nc3-c3ccccc3)c2cc1NC(=O)C(C)F] |
| | RCO₂H: 2-fluoropropanoic acid |
| | LCMS: m/z = 406 [M + H]⁺ |
| 222 | 4-fluoro-N-(7-methoxy-4-(1-methyl-3-phenyl-1H-pyrazol-4-yl)quinazolin-6- |
| | yl)benzamide |
| | 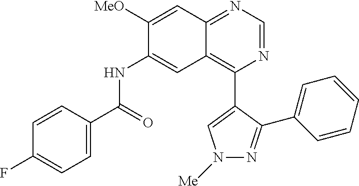 [SMILES: COc1cc2ncnc(-c3cn(C)nc3-c3ccccc3)c2cc1NC(=O)c1ccc(F)cc1] |
| | RCO₂H: 4-fluorobenzoic acid |
| | LCMS: m/z = 454 [M + H]⁺ |
| 223 | N-(7-methoxy-4-(1-methyl-3-phenyl-1H-pyrazol-4-yl)quinazolin-6-yl)-3- |
| | oxabicyclo[3.1.0]hexane-6-carboxamide |
| | 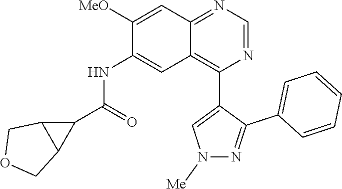 [SMILES: COc1cc2ncnc(-c3cn(C)nc3-c3ccccc3)c2cc1NC(=O)C1C2COCC21] |
| | RCO₂H: 3-oxabicyclo[3.1.0]hexane-6-carboxylic acid |
| | LCMS: m/z = 442 [M + H]⁺ |
| 224 | N-(7-methoxy-4-(1-methyl-3-phenyl-1H-pyrazol-4-yl)quinazolin-6-yl)-2- |
| | methyltetrahydrofuran-2-carboxamide |
| | 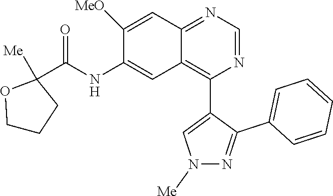 [SMILES: COc1cc2ncnc(-c3cn(C)nc3-c3ccccc3)c2cc1NC(=O)C1(C)CCCO1] |
| | RCO₂H: 2-methyltetrahydrofuran-2-carboxylic acid |
| | LCMS: m/z = 520 [M + H]⁺ |
| 225 | N-(7-methoxy-4-(1-methyl-3-phenyl-1H-pyrazol-4-yl)quinazolin-6-yl)-4,5,6,7- |
| | tetrahydropyrazolo[1,5-a]pyridine-2-carboxamide |
| | 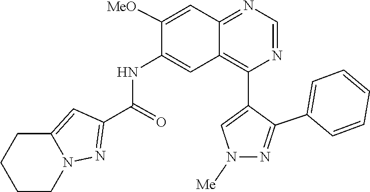 [SMILES: COc1cc2ncnc(-c3cn(C)nc3-c3ccccc3)c2cc1NC(=O)c1cc2n(n1)CCCC2] |
| | RCO₂H: 4,5,6,7-tetrahydropyrazolo[1,5-a]pyridine-2-carboxylic acid |
| | LCMS: m/z = 480 [M + H]⁺ |
| 226 | N-(7-methoxy-4-(1-methyl-3-phenyl-1H-pyrazol-4-yl)quinazolin-6-yl)-4,5,6,7- |
| | tetrahydropyrazolo[1,5-a]pyridine-3-carboxamide |
| | 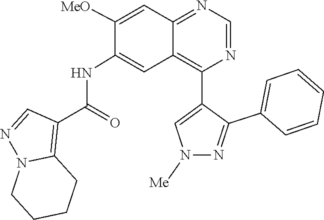 [SMILES: COc1cc2ncnc(-c3cn(C)nc3-c3ccccc3)c2cc1NC(=O)c1cnn2c1CCCC2] |
| | RCO₂H: 4,5,6,7-tetrahydropyrazolo[1,5-a]pyridine-3-carboxylic acid |
| | LCMS: m/z = 480 [M + H]⁺ |
| 227† | N-(7-methoxy-4-(1-methyl-3-phenyl-1H-pyrazol-4-yl)quinazolin-6-yl)-2- |
| | (piperazin-1-yl)benzamide |
| | 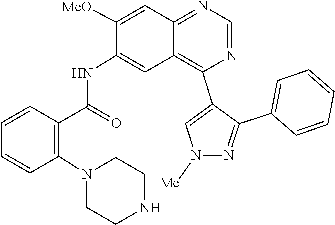 [SMILES: COc1cc2ncnc(-c3cn(C)nc3-c3ccccc3)c2cc1NC(=O)c1ccccc1N1CCNCC1] |
| | RCO₂H: 2-(4-(tert-butoxycarbonyl)piperazin-1-yl)benzoic acid |
| | LCMS: m/z = 520 [M + H]⁺ |
| 228† | N-(7-methoxy-4-(1-methyl-3-phenyl-1H-pyrazol-4-yl)quinazolin-6-yl)azetidine-2- |
| | carboxamide |
| | 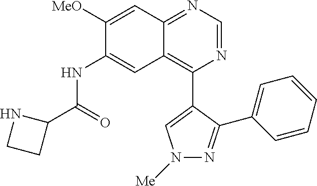 [SMILES: COc1cc2ncnc(-c3cn(C)nc3-c3ccccc3)c2cc1NC(=O)C1CCN1] |
| | RCO₂H: 1-(tert-butoxycarbonyl)azetidine-2-carboxylic acid |
| | LCMS: m/z = 415 [M + H]⁺ |

†Additional Boc deprotection required. To a solution of Boc protected intermediate (crude) in DCM (3.0 mL) was added TFA (1.0 mL). The mixture was stirred at 30° C. for 2 hrs. The mixture was concentrated under reduced pressure to give a residue. The residue was purified by prep-HPLC to give the title compounds.

### Example 229:6-cyclopropyl-N-(7-methoxy-4-(1-methyl-3-phenyl-1H-pyrazol-4-yl)quinazolin-6-yl) nicotinamide

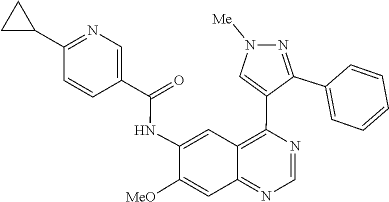 [SMILES: COc1cc2ncnc(-c3cn(C)nc3-c3ccccc3)c2cc1NC(=O)c1ccc(C2CC2)nc1]

Part 1: DMF (44.6 mg, 0.304 mmol) was added to a mixture of 6-cyclopropylnicotinic acid (500 mg, 3.04 mmol) and oxalic dichloride (771 mg, 6.08 mmol) in DCM at 0° C. The reaction was warmed to rt and stirred at 25° C. for 1 h. The mixture was evaporated to dryness in vacuo and used without further purification in the Part 2.

Part 2: Pyridine (35.7 mg, 0.452 mmol) was added dropwise to a mixture of 7-methoxy-4-(1-methyl-3-phenyl-1H-pyrazol-4-yl)quinazolin-6-amine (Preparation 190, 200 mg, 0.604 mmol) and 6-cyclopropylpyridine-3-carbonyl chloride (Part 1, 131 mg, 0.724 mmol) in DCM and reaction was stirred at rt for 1 h. The reaction was purified by prep-HPLC (XBridge Shield RP18 OBD Column, 30*150 mm, 5 μm; 35-50% MeCN/H₂O (10 mM NH₄HCO₃+0.1% NH₄OH) to afford the title compound as a white solid (144 mg, 50%). LCMS: m/z=477 [M+H]⁺; ¹H NMR (300 MHz, DMSO-d₆) δ: 9.77 (s, 1H), 9.06 (s, 1H), 8.88 (d, 1H), 8.54 (s, 1H), 8.20 (s, 1H), 8.09 (dd, 1H), 7.52-7.39 (m, 2H), 7.34-7.23 (m, 2H), 7.18 (ddt, 3H), 4.03 (d, 6H), 2.19 (tt, 1H), 1.09-0.93 (m, 4H).

### Example 230:6-cyclopropyl-N-(4-(3-(2-fluorophenyl)-1-methyl-1H-pyrazol-4-yl)-7-methoxyquinazolin-6-yl) nicotinamide

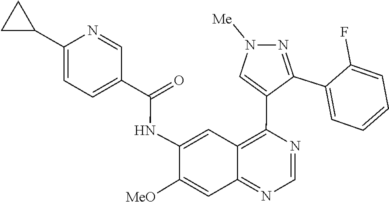 [SMILES: COc1cc2ncnc(-c3cn(C)nc3-c3ccccc3F)c2cc1NC(=O)c1ccc(C2CC2)nc1]

The title compound was prepared as a white solid (72.9 mg, 30%) from 4-(3-(2-fluorophenyl)-1-methyl-1H-pyrazol-4-yl)-7-methoxyquinazolin-6-amine (Preparation 253) and 6-cyclopropylnicotinic acid using an analogous 2-part process as described for Example 229, but using TEA as the base. LCMS: m/z=495 [M+H]⁺; ¹H NMR (300 MHz, DMSO-d₆) δ: 9.83 (s, 1H), 8.94 (s, 2H), 8.70 (s, 1H), 8.36 (s, 1H), 8.19-8.10 (m, 1H), 7.60 (s, 1H), 7.47 (d, 2H), 7.32 (s, 1H), 7.20 (d, 1H), 6.99 (s, 1H), 4.06 (s, 6H), 2.22 (s, 1H), 1.05 (d, 4H).

### Example 231: 2,6-dichloro-N-(7-methoxy-4-(1-methyl-3-phenyl-1H-pyrazol-4-yl)quinazolin-6-yl)benzamide

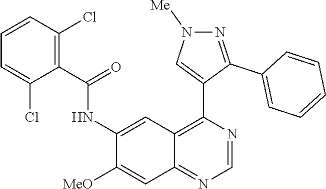 [SMILES: COc1cc2ncnc(-c3cn(C)nc3-c3ccccc3)c2cc1NC(=O)c1c(Cl)cccc1Cl]

The title compound was prepared as an off-white solid (27.4 mg, 22%) from 7-methoxy-4-(1-methyl-3-phenyl-1H-pyrazol-4-yl)quinazolin-6-amine (Preparation 190) and 2,6-dichlorobenzoic acid using an analogous 2-part process as described for Example 229. LCMS: m/z=504 [M+H]⁺; ¹H NMR (300 MHz, DMSO-d₆) δ: 10.39 (s, 1H), 9.11 (s, 1H), 8.75 (s, 1H), 8.24 (s, 1H), 7.60-7.43 (m, 4H), 7.34-7.23 (m, 2H), 7.23-7.14 (m, 3H), 4.04 (d, 6H).

### Example 232: N-(7-ethoxy-4-(1-methyl-3-phenyl-1H-pyrazol-4-yl)quinazolin-6-yl)-1-(trifluoromethyl)-1H-pyrazole-4-carboxamide

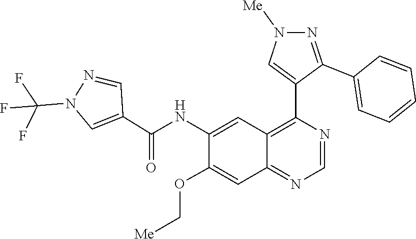 [SMILES: CCOc1cc2ncnc(-c3cn(C)nc3-c3ccccc3)c2cc1NC(=O)c1cnn(C(F)(F)F)c1]

A mixture of 1-(trifluoromethyl)-1H-pyrazole-4-carboxamide (Preparation 76, 30 mg, 0.168 mmol), 6-bromo-7-ethoxy-4-(1-methyl-3-phenyl-1H-pyrazol-4-yl)quinazoline (Preparation 90, 82.3 mg, 0.201 mmol), K₂CO₃ (46.6 mg, 0.335 mmol), XPhos (7.99 mg, 0.017 mmol), Pd₂(dba)₃ (15.3 mg, 0.017 mmol) in dioxane (5 mL) was purged with N₂ and stirred for 3 h at 100° C. under N₂. The reaction was quenched by the addition of 5 mL of water and the resulting mixture extracted with EtOAc (3×10 mL). The combined extracts were evaporated to dryness in vacuo and the residue purified by column chromatography (20:1 DCM/MeOH) followed by prep-HPLC Column: XBridge Shield RP18 OBD Column, 30×150 mm, 5 μm; 8-56% MeCN/H₂O (10 mM NH₄HCO₃+0.1% NH₄OH) to afford the title compound as an off-white solid (9.3 mg, 11%). LCMS: m/z=508 [M+H]⁺; ¹H NMR (300 MHz, DMSO-d₆) δ: 9.63 (s, 1H), 9.18 (s, 1H), 9.08 (s, 1H), 8.46 (d, 2H), 8.21 (s, 1H), 7.50 (s, 1H), 7.30 (dd, 2H), 7.21 (q, 3H), 4.37 (q, 2H), 4.03 (s, 3H), 1.48 (t, 3H).

### Example 233:2-(4-(7-methoxy-4-(1-methyl-3-phenyl-1H-pyrazol-4-yl)quinazolin-6-yl)-1H-pyrazol-1-yl)-N,N-dimethylethan-1-amine

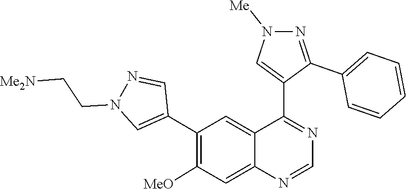 [SMILES: COc1cc2ncnc(-c3cn(C)nc3-c3ccccc3)c2cc1-c1cnn(CCN(C)C)c1]

A mixture of 6-bromo-7-methoxy-4-(1-methyl-3-phenyl-1H-pyrazol-4-yl)quinazoline (Preparation 91, 100 mg, 0.252 mmol), N,N-dimethyl-2-(4-(4,4,5,5-tetramethyl-1,3,2-dioxaborolan-2-yl)-1H-pyrazol-1-yl)ethan-1-amine (133 mg, 0.504 mmol), Pd(PPh₃)₄ (15 mg, 12.9 μmol) and K₂CO₃ (69.6 mg, 0.504 mmol) in dioxane (8 mL) and H₂O (2 mL) was stirred at 100° C. for 2 h. The reaction mixture was evaporated to dryness and the residue purified by prep-TLC (15:1 DCM/MeOH) and prep-HPLC (YMC-Actus Triart C18 ExRS, 30×150 mm, 5 μm; 21-51% MeCN/H₂O (10 mM NH₄HCO₃+0.1% NH₄OH)) to afford the title compound as a white solid (60.7 mg, 53%). LCMS: m/z=454 [M+H]⁺; ¹H NMR (300 MHz, DMSO-d₆) δ: 9.08 (s, 1H), 8.30 (s, 1H), 7.95 (d, 1H), 7.83 (s, 1H), 7.49 (d, 1H), 7.43 (s, 1H), 7.35 (m, 2H), 7.28-7.19 (m, 3H), 4.19 (t, 2H), 4.05 (d, 6H), 2.64 (t, 2H), 2.17 (s, 6H).

### Example 234 and 235: N-(7-methoxy-4-(1-methyl-3-phenyl-1H-pyrazol-4-yl)quinazolin-6-yl)-5-methyl-1-(trifluoromethyl)-1H-pyrazole-4-carboxamide and N-(7-methoxy-4-(1-methyl-3-phenyl-1H-pyrazol-4-yl)quinazolin-6-yl)-3-methyl-1-(trifluoromethyl)-1H-pyrazole-4-carboxamide

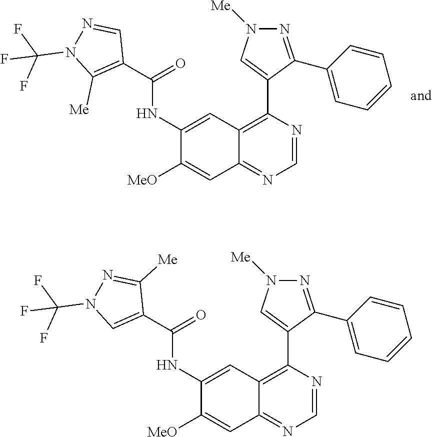 [SMILES: COc1cc2ncnc(-c3cn(C)nc3-c3ccccc3)c2cc1NC(=O)c1cnn(C(F)(F)F)c1C ; COc1cc2ncnc(-c3cn(C)nc3-c3ccccc3)c2cc1NC(=O)c1cn(C(F)(F)F)nc1C]

A mixture of 7-methoxy-4-(1-methyl-3-phenyl-1H-pyrazol-4-yl)quinazolin-6-amine (Preparation 190, 150 mg, 452 μmol) and 3-methyl-1-(trifluoromethyl)-1H-pyrazole-4-carboxylic acid and 5-methyl-1-(trifluoromethyl)-1H-pyrazole-4-carboxylic acid (Preparation 85, 262 mg, 1.35 mmol) and T3P® (3 mL) and pyridine (3 mL) in THF (4 mL) at rt was heated to 50° C. for 2 h. The mixture was evaporated to dryness in vacuo and the residue purified by prep-HPLC (XBridge Shield RP18 OBD, 30×150 mm, 5 μm; 27-63% MeCN/H₂O (10 mM NH₄HCO₃+0.1% NH₄OH)) to afford a mixture of This resulted in a mixture (170 mg, 74%) of N-(7-methoxy-4-(1-methyl-3-phenyl-1H-pyrazol-4-yl)quinazolin-6-yl)-5-methyl-1-(trifluoromethyl)-1H-pyrazole-4-carboxamide and N-(7-methoxy-4-(1-methyl-3-phenyl-1H-pyrazol-4-yl)quinazolin-6-yl)-3-methyl-1-(trifluoromethyl)-1H-pyrazole-4-carboxamide. The mixture of title compounds were isolated by prep-SFC chromatography (Diacel DCpak P4VP, 30×250 mm, 5 μm; 25% IPA (0.5% 2M NH₃—MeOH) in CO₂) to afford:

Peak 1, white solid (26.1 mg, 15%). Example 234, N-(7-methoxy-4-(1-methyl-3-phenyl-1H-pyrazol-4-yl)quinazolin-6-yl)-5-methyl-1-(trifluoromethyl)-1H-pyrazole-4-carboxamide. LCMS: m/z=508 [M+H]⁺; ¹H NMR (300 MHZ, DMSO-d₆) δ: 9.63 (s, 1H), 9.09 (s, 1H), 8.43 (d, 2H), 8.23 (d, 1H), 7.51 (s, 1H), 7.30-7.21 (m, 5H), 4.05 (d, 6H), 2.69 (d, 3H).

Peak 2, white solid (69.1 mg, 41%). Example 235, N-(7-methoxy-4-(1-methyl-3-phenyl-1H-pyrazol-4-yl)quinazolin-6-yl)-3-methyl-1-(trifluoromethyl)-1H-pyrazole-4-carboxamide. LCMS: m/z=508 [M+H]⁺; ¹H NMR (400 MHZ, DMSO-d₆) δ: 9.49 (s, 1H), 9.10 (d, 2H), 8.53 (s, 1H), 8.22 (s, 1H), 7.50 (s, 1H), 7.29 (ddt, 2H), 7.24-7.16 (m, 3H), 4.08-4.03 (m, 6H), 2.43 (s, 3H).

### Example 236 and 237: (S)-2-(dimethylamino)-N-(7-methoxy-4-(1-methyl-3-phenyl-1H-pyrazol-4-yl)quinazolin-6-yl) propenamide and (R)-2-(dimethylamino)-N-(7-methoxy-4-(1-methyl-3-phenyl-1H-pyrazol-4-yl)quinazolin-6-yl) propanamide

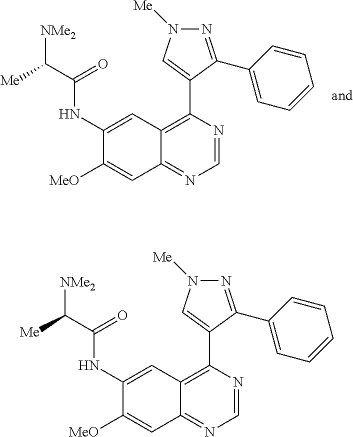 [SMILES: COc1cc2ncnc(-c3cn(C)nc3-c3ccccc3)c2cc1NC(=O)[C@H](C)N(C)C ; COc1cc2ncnc(-c3cn(C)nc3-c3ccccc3)c2cc1NC(=O)[C@@H](C)N(C)C]

Part 1. T3P® (2 mL) and pyridine (2 mL) were added to 7-methoxy-4-(1-methyl-3-phenyl-1H-pyrazol-4-yl)quinazolin-6-amine (Preparation 190, 200 mg, 0.603 mmol) and 2-bromopropanoic acid (275 mg, 1.80 mmol) in THF (2 mL) at rt and the resulting mixture heated to 50° C. for 16 h. The reaction mixture was diluted with EtOAc (100 mL) and washed with water (3×100 mL) and brine (100 mL). The organic layer was dried (Na₂SO₄) and evaporated to dryness and the residue purified by prep-TLC (20:1 DCM/MeOH) to afford 2-bromo-N-(7-methoxy-4-(1-methyl-3-phenyl-1H-pyrazol-4-yl)quinazolin-6-yl) propenamide was prepared as a yellow solid (130 mg, 46%). LCMS: m/z=466 [M+H]⁺.

Part 2. Cs₂CO₃ (181 mg, 556 μmol) was added to bromo-N-(7-methoxy-4-(1-methyl-3-phenyl-1H-pyrazol-4-yl)quinazolin-6-yl) propenamide (Part 1, 130 mg, 278 μmol) and dimethylamine (1 mL) in DMF (5 mL) at rt and the resulting mixture heated to 40° C. for 16 h. The mixture was evaporated to dryness in vacuo and the residue diluted with EtOAc (100 mL) and washed with water (3×100 mL) and saturated brine (100 mL). The combined organics were dried (Na₂SO₄) and evaporated to dryness. The residue was purified by prep-TLC (15:1 DCM/MeOH) followed by prep-HPLC (YMC-Actus Triart C18 ExRS, 30×150 mm, 5 μm; 25-58% MeCN/H₂O (10 mM NH₄HCO₃+0.1% NH₄OH)) to afford 2-(dimethylamino)-N-(7-methoxy-4-(1-methyl-3-phenyl-1H-pyrazol-4-yl)quinazolin-6-yl) propenamide as a white solid (80 mg, 66%). LCMS: m/z=431 [M+H]⁺.

Part 3. 2-(dimethylamino)-N-(7-methoxy-4-(1-methyl-3-phenyl-1H-pyrazol-4-yl)quinazolin-6-yl) propenamide (Part 3, 80 mg, 0.185 mmol) was separated using prep-HPLC (CHIRALPAK IG, 20×250 mm, 5 μm; 50% EtOH/Hex (0.5% 2M NH₃—MeOH)) to afford the title compounds.

Peak 1, Example 236 (white solid, 36.4 mg, 46%). (S)-2-(dimethylamino)-N-(7-methoxy-4-(1-methyl-3-phenyl-1H-pyrazol-4-yl)quinazolin-6-yl) propenamide or (R)-2-(dimethylamino)-N-(7-methoxy-4-(1-methyl-3-phenyl-1H-pyrazol-4-yl)quinazolin-6-yl) propenamide. LCMS: m/z=431 [M+H]⁺; ¹H NMR (300 MHz, DMSO-d₆) δ: 9.85 (s, 1H), 9.05 (s, 1H), 8.81 (s, 1H), 8.21 (s, 1H), 7.47 (s, 1H), 7.30-7.18 (m, 5H), 4.06 (d, 6H), 3.25 (t, 1H), 2.23 (s, 6H), 1.12 (d, 3H).

Peak 2, Example 237 (white solid, 33.4 mg, 42%). (R)-2-(dimethylamino)-N-(7-methoxy-4-(1-methyl-3-phenyl-1H-pyrazol-4-yl)quinazolin-6-yl) propenamide or (S)-2-(dimethylamino)-N-(7-methoxy-4-(1-methyl-3-phenyl-1H-pyrazol-4-yl)quinazolin-6-yl) propenamide. LCMS: m/z=431 [M+H]⁺; ¹H NMR (300 MHz, DMSO-d₆) δ: 9.85 (s, 1H), 9.05 (s, 1H), 8.81 (s, 1H), 8.21 (s, 1H), 7.47 (s, 1H), 7.30-7.19 (m, 5H), 4.06 (d, 6H), 3.26 (q, 1H), 2.23 (s, 6H), 1.12 (d, 3H).

### Example 238 and 239: (S)-3-(dimethylamino)-N-(7-methoxy-4-(1-methyl-3-phenyl-1H-pyrazol-4-yl)quinazolin-6-yl)-2-methylpropanamide and (R)-3-(dimethylamino)-N-(7-methoxy-4-(1-methyl-3-phenyl-1H-pyrazol-4-yl)quinazolin-6-yl)-2-methylpropanamide

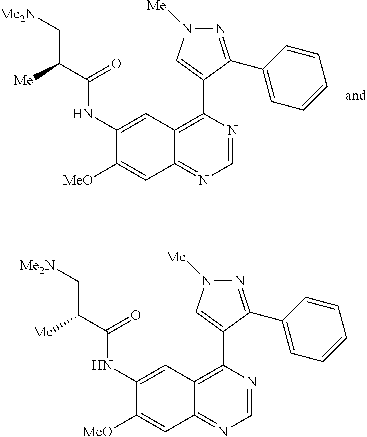 [SMILES: COc1cc2ncnc(-c3cn(C)nc3-c3ccccc3)c2cc1NC(=O)[C@@H](C)CN(C)C ; COc1cc2ncnc(-c3cn(C)nc3-c3ccccc3)c2cc1NC(=O)[C@H](C)CN(C)C]

Part 1. 3-(dimethylamino)-N-(7-methoxy-4-(1-methyl-3-phenyl-1H-pyrazol-4-yl)quinazolin-6-yl)-2-methylpropanamide was prepared from 7-methoxy-4-(1-methyl-3-phenyl-1H-pyrazol-4-yl)quinazolin-6-amine (Preparation 190) and 3-(dimethylamino)-2-methylpropanoic acid using an analogous method to that described for Example 187. LCMS: m/z=445 [M+H]⁺.

Part 2. 3-(dimethylamino)-N-(7-methoxy-4-(1-methyl-3-phenyl-1H-pyrazol-4-yl)quinazolin-6-yl)-2-methylpropanamide (Part 1) was separated by chiral-HPLC (CHIRAL ART Cellulose-SB, 20×250 mm, 5 μm; 20% IPA/Hex (0.5% 2M NH₃—MeOH)) to afford the title compounds.

Peak 1, Example 238 (white solid, 37.1 mg). (S)-3-(dimethylamino)-N-(7-methoxy-4-(1-methyl-3-phenyl-1H-pyrazol-4-yl)quinazolin-6-yl)-2-methylpropanamide or (R)-3-(dimethylamino)-N-(7-methoxy-4-(1-methyl-3-phenyl-1H-pyrazol-4-yl)quinazolin-6-yl)-2-methylpropanamide. LCMS: m/z=445 [M+H]⁺; ¹H NMR (400 MHZ, DMSO-d₆) δ: 11.60 (s, 1H), 9.01 (s, 1H), 8.86 (s, 1H), 8.15 (s, 1H), 7.42 (s, 1H), 7.27 (dtd, 2H), 7.23-7.15 (m, 3H), 4.08 (s, 6H), 2.73 (ddd, 1H), 2.59 (t, 1H), 2.30 (s, 6H), 2.19 (dd, 1H), 1.01 (d, 3H).

Peak 2, Example 239 (white solid, 29.1 mg). (R)-3-(dimethylamino)-N-(7-methoxy-4-(1-methyl-3-phenyl-1H-pyrazol-4-yl)quinazolin-6-yl)-2-methylpropanamide or (S)-3-(dimethylamino)-N-(7-methoxy-4-(1-methyl-3-phenyl-1H-pyrazol-4-yl)quinazolin-6-yl)-2-methylpropanamide. LCMS: m/z=445 [M+H]⁺; ¹H NMR (400 MHZ, DMSO-d₆) δ: 11.61 (s, 1H), 9.01 (s, 1H), 8.86 (s, 1H), 8.15 (s, 1H), 7.42 (s, 1H), 7.27 (dtd, 2H), 7.23-7.15 (m, 3H), 4.08 (s, 6H), 2.73 (ddd, 1H), 2.59 (t, 1H), 2.30 (s, 6H), 2.19 (dd, 1H), 1.01 (d, 3H).

### Example 240 and 241: (1R,5R)—N-(4-(3-(2-fluorophenyl)-1-methyl-1H-pyrazol-4-yl)-7-methoxyquinazolin-6-yl)-3-oxabicyclo[3.1.0]hexane-1-carboxamide and (1S,5S)—N-(4-(3-(2-fluorophenyl)-1-methyl-1H-pyrazol-4-yl)-7-methoxyquinazolin-6-yl)-3-oxabicyclo[3.1.0]hexane-1-carboxamide

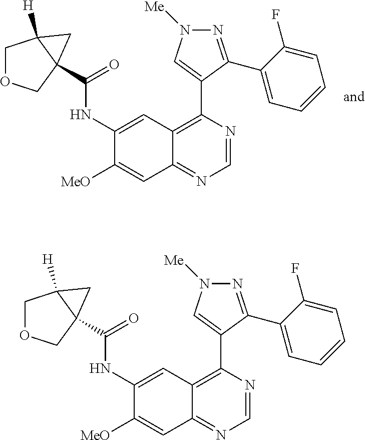 [SMILES: COc1cc2ncnc(-c3cn(C)nc3-c3ccccc3F)c2cc1NC(=O)[C@@]12COC[C@@H]1C2 ; COc1cc2ncnc(-c3cn(C)nc3-c3ccccc3F)c2cc1NC(=O)[C@]12COC[C@H]1C2]

The title compounds were prepared from 4-(3-(2-fluorophenyl)-1-methyl-1H-pyrazol-4-yl)-7-methoxyquinazolin-6-amine (Preparation 253) and 3-oxabicyclo[3.1.0]hexane-1-carboxylic acid using an analogous 2-part procedure as described for Examples 238 and 239. The racemic product was purified by Chiral-HPLC (CHIRAL ART Amylose-C NEO, 20×250 mm, 5 μm; 30% IPA/Hex (0.5% 2M NH₃—MeOH)) to afford:

Peak 1, Example 240 (off-white solid, 136.2 mg, 45%). (1R,5R)—N-(4-(3-(2-fluorophenyl)-1-methyl-1H-pyrazol-4-yl)-7-methoxyquinazolin-6-yl)-3-oxabicyclo[3.1.0]hexane-1-carboxamide or (1S,5S)—N-(4-(3-(2-fluorophenyl)-1-methyl-1H-pyrazol-4-yl)-7-methoxyquinazolin-6-yl)-3-oxabicyclo[3.1.0]hexane-1-carboxamide. LCMS: m/z=460 [M+H]⁺; ¹H NMR (300 MHz, DMSO-d₆) δ: 8.90 (s, 1H), 8.71 (s, 1H), 8.56 (s, 1H), 8.30 (s, 1H), 7.57 (td, 1H), 7.42 (s, 1H), 7.30 (ddd, 1H), 7.18 (dd, 1H), 6.96 (ddd, 1H), 4.04 (s, 5H), 3.97 (s, 2H), 3.73 (d, 2H), 2.19 (ddd, 1H), 1.41 (dd, 1H), 0.89 (t, 1H).

Peak 2, Example 241 (off-white solid, 134.5 mg, 45%). (1S,5S)—N-(4-(3-(2-fluorophenyl)-1-methyl-1H-pyrazol-4-yl)-7-methoxyquinazolin-6-yl)-3-oxabicyclo[3.1.0]hexane-1-carboxamide or (1R,5R)—N-(4-(3-(2-fluorophenyl)-1-methyl-1H-pyrazol-4-yl)-7-methoxyquinazolin-6-yl)-3-oxabicyclo[3.1.0]hexane-1-carboxamide. LCMS: m/z=460 [M+H]⁺; ¹H NMR (300 MHz, DMSO-d₆) δ: 8.90 (s, 1H), 8.71 (s, 1H), 8.56 (s, 1H), 8.30 (s, 1H), 7.57 (td, 1H), 7.42 (s, 1H), 7.37-7.25 (m, 1H), 7.19 (td, 1H), 6.96 (ddd, 1H), 4.04 (s, 5H), 3.97 (s, 2H), 3.73 (d, 2H), 2.18 (td, 1H), 1.41 (dd, J=8.3, 4.4 Hz, 1H), 0.89 (t, 1H).

### Example 242 and 243: (R)—N-(7-methoxy-4-(1-methyl-3-phenyl-1H-pyrazol-4-yl)quinazolin-6-yl)-1-methylpyrrolidine-2-carboxamide and (S)—N-(7-methoxy-4-(1-methyl-3-phenyl-1H-pyrazol-4-yl)quinazolin-6-yl)-1-methylpyrrolidine-2-carboxamide

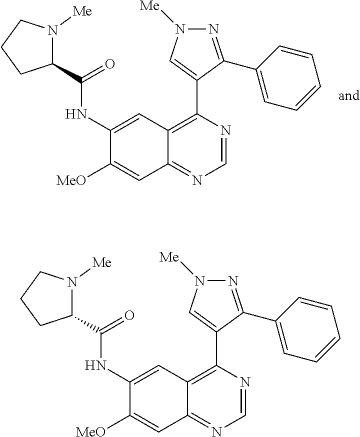 [SMILES: COc1cc2ncnc(-c3cn(C)nc3-c3ccccc3)c2cc1NC(=O)[C@H]1CCCN1C ; COc1cc2ncnc(-c3cn(C)nc3-c3ccccc3)c2cc1NC(=O)[C@@H]1CCCN1C]

The title compounds were prepared from 7-methoxy-4-(1-methyl-3-phenyl-1H-pyrazol-4-yl)quinazolin-6-amine (Preparation 190) and 1-methylpyrrolidine-2-carboxylic acid using an analogous 2-part procedure as described for Examples 240 and 241. The racemic product was purified by Chiral-HPLC (Chiralpak IG, 20×250 mm, 5 μm; 50% EtOH/Hex (0.5% 2M NH₃—MeOH)) to afford:

Peak 1, Example 242 (white solid, 27.7 mg, 35%). (R)—N-(7-methoxy-4-(1-methyl-3-phenyl-1H-pyrazol-4-yl)quinazolin-6-yl)-1-methylpyrrolidine-2-carboxamide or (S)—N-(7-methoxy-4-(1-methyl-3-phenyl-1H-pyrazol-4-yl)quinazolin-6-yl)-1-methylpyrrolidine-2-carboxamide: LCMS: m/z=443 [M+H]⁺; ¹H NMR (300 MHz, DMSO-d₆) δ: 9.92 (s, 1H), 9.05 (s, 1H), 8.83 (s, 1H), 8.22 (s, 1H), 7.48 (s, 1H), 7.27-7.14 (m, 5H), 4.10-4.04 (m, 6H), 3.15 (t, 1H), 2.95 (dd, 1H), 2.39 (s, 4H), 2.17 (s, 1H), 1.80-1.68 (m, 3H).

Peak 2, Example 243 (white solid, 30 mg, 37%). (R)—N-(7-methoxy-4-(1-methyl-3-phenyl-1H-pyrazol-4-yl)quinazolin-6-yl)-1-methylpyrrolidine-2-carboxamide or (S)—N-(7-methoxy-4-(1-methyl-3-phenyl-1H-pyrazol-4-yl)quinazolin-6-yl)-1-methylpyrrolidine-2-carboxamide: LCMS: m/z=443 [M+H]⁺; ¹H NMR (300 MHz, DMSO-d₆): δ 9.92 (s, 1H), 9.05 (s, 1H), 8.83 (s, 1H), 8.22 (s, 1H), 7.48 (s, 1H), 7.26-7.14 (m, 5H), 4.10-4.04 (s, 6H), 3.15 (t, 1H), 2.95 (dd, 1H), 2.39 (s, 4H), 2.17 (s, 1H), 1.80-1.68 (m, 3H).

### Example 244 and 245: (S)-2-fluoro-N-(7-methoxy-4-(1-methyl-3-phenyl-1H-pyrazol-4-yl)quinazolin-6-yl) propenamide and (R)-2-fluoro-N-(7-methoxy-4-(1-methyl-3-phenyl-1H-pyrazol-4-yl)quinazolin-6-yl) propanamide

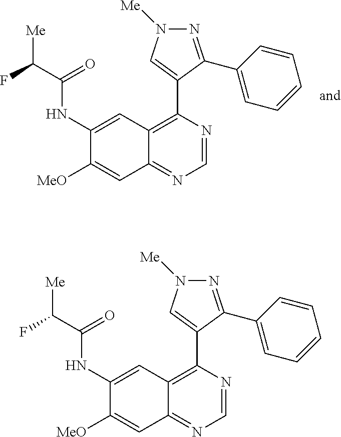 [SMILES: COc1cc2ncnc(-c3cn(C)nc3-c3ccccc3)c2cc1NC(=O)[C@H](C)F ; COc1cc2ncnc(-c3cn(C)nc3-c3ccccc3)c2cc1NC(=O)[C@@H](C)F]

The title compounds were obtained from 2-fluoro-N-(7-methoxy-4-(1-methyl-3-phenyl-1H-pyrazol-4-yl)quinazolin-6-yl) propenamide (Example 221) by prep-HPLC (CHIRALPAK IG, 20×250 mm, 5 μm; 7% EtOH/3:1 Hex/DCM (0.5% 2M NH₃—MeOH).

Peak 1, Example 244 (white solid, 4.5 mg) (S)-2-fluoro-N-(7-methoxy-4-(1-methyl-3-phenyl-1H-pyrazol-4-yl)quinazolin-6-yl) propenamide or (R)-2-fluoro-N-(7-methoxy-4-(1-methyl-3-phenyl-1H-pyrazol-4-yl)quinazolin-6-yl) propenamide. LCMS: m/z=406 [M+H]⁺; ¹H NMR (400 MHz, DMSO-d₆) δ: 9.31 (d, 1H), 9.08 (s, 1H), 8.69 (s, 1H), 8.21 (s, 1H), 7.49 (s, 1H), 7.26 (p, 5H), 5.38 (p, 1H), 4.09 (s, 6H), 1.53 (d, 3H), 1.46 (d, 3H).

Peak 2, Example 245 (white solid, 1.9 mg). (R)-2-fluoro-N-(7-methoxy-4-(1-methyl-3-phenyl-1H-pyrazol-4-yl)quinazolin-6-yl) propenamide or (S)-2-fluoro-N-(7-methoxy-4-(1-methyl-3-phenyl-1H-pyrazol-4-yl)quinazolin-6-yl) propenamide. LCMS: m/z=406 [M+H]⁺; ¹H NMR (400 MHz, DMSO-d₆) δ: 9.31 (d, 1H), 9.08 (s, 1H), 8.69 (s, 1H), 8.21 (s, 1H), 7.49 (s, 1H), 7.26 (p, 5H), 5.38 (p, 1H), 4.09 (s, 6H), 1.53 (d, 3H), 1.46 (d, 3H).

### Example 246:2-(dimethylamino)-N-(7-methoxy-4-(1-methyl-3-phenyl-1H-pyrazol-4-yl)quinazolin-6-yl)-2-methylpropanamide

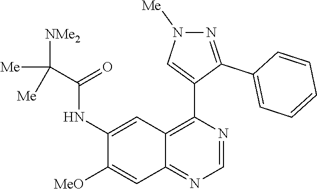 [SMILES: COc1cc2ncnc(-c3cn(C)nc3-c3ccccc3)c2cc1NC(=O)C(C)(C)N(C)C]

The title compound was prepared as a white solid (14.2 mg, 8%) from 7-methoxy-4-(1-methyl-3-phenyl-1H-pyrazol-4-yl)quinazolin-6-amine (Preparation 190), 2-bromo-2-methylpropanoic acid and dimethylamine using an analogous 2-part method to that described for Example 236 (Part 1 and Part 2). Purified by prep-HPLC (XBridge Shield RP18 OBD Column, 30×150 mm, 5 μm; 37-47% MeCN/H₂O (10 mM NH₄HCO₃+0.1% NH₄OH)). LCMS: m/z=445 [M+H]⁺; ¹H NMR (300 MHz, DMSO-d₆) δ: 9.94 (s, 1H), 9.05 (s, 1H), 8.77 (s, 1H), 8.22 (s, 1H), 7.47 (s, 1H), 7.29-7.18 (m, 5H), 4.07 (d, 6H), 2.18 (s, 6H), 1.13 (s, 6H).

### Example 247: N-(7-methoxy-4-(3-phenyl-1H-pyrazol-4-yl)quinazolin-6-yl)propionamide

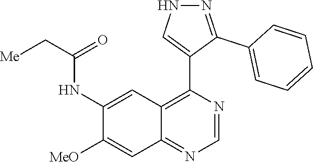 [SMILES: CCC(=O)Nc1cc2c(-c3c[nH]nc3-c3ccccc3)ncnc2cc1OC]

A solution of N-(7-methoxy-4-(3-phenyl-1-(tetrahydro-2H-pyran-2-yl)-1H-pyrazol-4-yl)quinazolin-6-yl)propionamide (Preparation 206, 20 mg, 43.7 μmol) in HCl (4M in dioxane, 1 mL) was stirred at rt for 1 h. The reaction mixture was concentrated in vacuo and the residue purified by prep-HPLC (XSelect CSH Prep C18 OBD, 19×250 mm, 5 μm; 25-60% MeCN/H₂O (0.1% HCO₂H)) afforded the title compound as a yellow solid (3.7 mg, 22%). LCMS: m/z=374 [M+H]⁺; ¹H NMR (300 MHz, DMSO-d₆) δ: 13.4 (s, 1H), 9.34 (s, 1H), 8.99 (s, 1H), 8.79 (s, 1H), 7.41 (s, 1H), 7.32 (dd, 2H), 7.24 (s, 3H), 4.05 (s, 3H), 2.40 (t, 2H), 1.02 (t, 3H).

### Example 248: N-(7-methoxy-4-(3-phenyl-1-(tetrahydro-2H-pyran-4-yl)-1H-pyrazol-4-yl)quinazolin-6-yl)propionamide

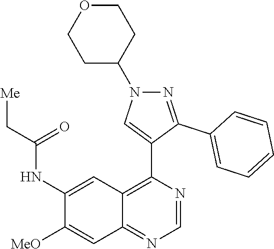 [SMILES: CCC(=O)Nc1cc2c(-c3cn(C4CCOCC4)nc3-c3ccccc3)ncnc2cc1OC]

A mixture of N-(7-methoxy-4-(3-phenyl-1H-pyrazol-4-yl)quinazolin-6-yl)propionamide (Example 247, 120 mg, 0.321 mmol), 4-iodotetrahydro-2H-pyran (68 mg, 0.321 mmol), Cs₂CO₃ (209 mg, 0.642 mmol) in DMF (4 mL) was stirred at 80° C. for 2 h. The reaction mixture was extracted with EtOAc (3×20 mL) and the combined organics were dried (Na₂SO₄) and evaporated to dryness in vacuo. The residue was purified by prep-HPLC (CHIRAL ART Cellulose-SC, 20×250 mm, 5 μm; 50% EtOH/Hex (0.5% 2M NH₃—MeOH) to afford the title compound as a white solid (14.5 mg, 10%). LCMS: m/z=458 [M+H]⁺; ¹H NMR (300 MHz, DMSO-d₆) δ: 9.35 (s, 1H), 9.03 (s, 1H), 8.79 (s, 1H), 8.31 (s, 1H), 7.44 (s, 1H), 7.37-7.27 (m, 2H), 7.22 (ddt, 3H), 4.61 (tt, 1H), 4.07 (s, 3H), 4.01 (s, 1H), 3.55 (td, 2H), 2.43 (q, 2H), 2.19-2.00 (m, 4H), 1.04 (t, 3H).

### Example 249-250

The title compounds were prepared from N-(7-methoxy-4-(3-phenyl-1H-pyrazol-4-yl)quinazolin-6-yl)propionamide (Example 247) and the appropriate halide using an analogous method to that described for Example 248.

| Example Number | Name/Structure/Halide/Data |
|---|---|
| 249 | N-(7-methoxy-4-(1-(oxetan-3-yl)-3-phenyl-1H-pyrazol-4-yl)quinazolin- |
| | 6-yl)propionamide |
| | 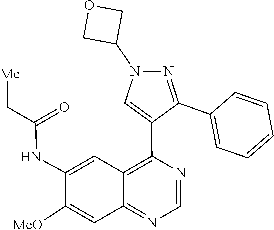 [SMILES: CCC(=O)Nc1cc2c(-c3cn(C4COC4)nc3-c3ccccc3)ncnc2cc1OC] |
| | Halide: 3-iodooxetane |
| | prep-HPLC (CHIRAL ART Cellulose-SC, 20 x 250 mm, 5 μm; 50% |
| | EtOH/Hex (0.5% 2M NH₃—MeOH) |
| | White solid (40.4 mg, 29%). LCMS: m/z = 430 [M + H]⁺; ¹H NMR (300 |
| | MHz, DMSO-d₆) δ: 9.35 (s, 1H), 8.76 (s, 2H), 8.42 (s, 1H), 7.45 (s, 1H), |
| | 7.39-7.29 (m, 2H), 7.24 (tt, 3H), 5.85-5.69 (m, 1H), 5.06-4.81 (p, 4H), |
| | 4.07 (s, 3H), 2.43 (q, 2H), 1.04 (t, 3H). |
| 250 | N-(4-(1-(2,2-difluoroethyl)-3-phenyl-1H-pyrazol-4-yl)-7- |
| | methoxyquinazolin-6-yl)propionamide |
| | 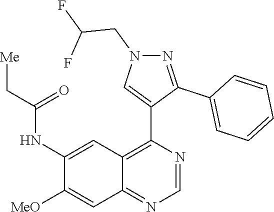 [SMILES: CCC(=O)Nc1cc2c(-c3cn(CC(F)F)nc3-c3ccccc3)ncnc2cc1OC] |
| | Halide: 2-bromo-1,1-difluoroethane |
| | prep-HPLC (CHIRAL ART Cellulose-SC, 20 x 250 mm, 5 μm; 50% |
| | EtOH/Hex (0.5% 2M NH₃—MeOH) |
| | White solid (13.1 mg, 9%). LCMS: m/z = 438 [M + H]⁺; ¹H NMR (300 |
| | MHz, DMSO-d₆) δ: 9.33 (s, 1H), 9.05 (s, 1H), 8.71 (s, 1H), 8.29 (s, 1H), |
| | 7.45 (s, 1H), 7.34-7.17 (m, 5H), 6.54 (t, 1H), 4.84 (td, 2H), 4.07 (s, 3H), |
| | 2.42 (q, 2H), 1.03 (t, 3H). |

### Example 251: N-(7-methoxy-4-(1-methyl-3-phenyl-1H-pyrazol-4-yl)quinazolin-6-yl)-1-methylazetidine-2-carboxamide

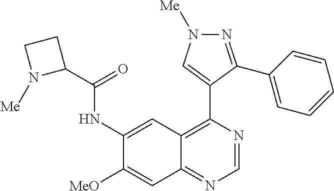 [SMILES: COc1cc2ncnc(-c3cn(C)nc3-c3ccccc3)c2cc1NC(=O)C1CCN1C]

Part 1. tert-Butyl 2-((7-methoxy-4-(1-methyl-3-phenyl-1H-pyrazol-4-yl)quinazolin-6-yl)carbamoyl) azetidine-1-carboxylate was prepared as a yellow solid (130 mg, 84%) from 7-methoxy-4-(1-methyl-3-phenyl-1H-pyrazol-4-yl)quinazolin-6-amine (Preparation 190) and 1-(tert-butoxycarbonyl) azetidine-2-carboxylic acid using an analogous method to that described for Example 236 and 237, Part 1. LCMS: m/z=515 [M+H]⁺.

Part 2. A solution of tert-butyl 2-((7-methoxy-4-(1-methyl-3-phenyl-1H-pyrazol-4-yl)quinazolin-6-yl)carbamoyl) azetidine-1-carboxylate (Part 1, 130 mg, 0.252 mmol) in TFA (1 mL) and DCM (2 mL) was stirred at 20° C. for 2 h. The mixture was extracted with EtOAc (3×20 mL) and the combined organics washed with brine (10 mL), dried (Na₂SO₄) and evaporated to dryness in vacuo. The residue was purified by prep-TLC (10:1 DCM/MeOH) to afford N-(7-methoxy-4-(1-methyl-3-phenyl-1H-pyrazol-4-yl)quinazolin-6-yl) azetidine-2-carboxamide as a yellow solid (100 mg, 96%). LCMS: m/z=415 [M+H]⁺.

Part 3. HCHO (97.2 mg, 1.20 mmol, 37% purity) was added to a solution of N-(7-methoxy-4-(1-methyl-3-phenyl-1H-pyrazol-4-yl)quinazolin-6-yl) azetidine-2-carboxamide (Part 2, 100 mg, 0.241 mmol) in MeOH (3 mL) and the mixture stirred at 20° C. for 20 min. To this was added NaBH(OAc)₃ (153 mg, 0.722 mmol) and the mixture stirred at 20° C. for 1 h and then evaporated to dryness. The residue was purified by prep-HPLC (XBridge Shield RP18 OBD, 30×150 mm, 5 μm; 19-55% MeCN/H₂O (10 mM NH₄HCO₃+0.1% NH₄OH)) to afford the title compound as a white solid (10 mg, 10%). LCMS: m/z=438 [M+H]⁺; ¹H NMR (400 MHZ, DMSO-d₆) δ: 9.94 (s, 1H), 9.06 (s, 1H), 8.86 (s, 1H), 8.21 (s, 1H), 7.50 (s, 1H), 7.30-7.23 (m, 2H), 7.20 (q, 3H), 4.12 (s, 3H), 4.03 (s, 3H), 3.55 (s, 1H), 2.98 (s, 1H), 2.35 (s, 3H), 2.01 (s, 1H).

### Example 252 and 253: (S)—N-(7-methoxy-4-(1-methyl-3-phenyl-1H-pyrazol-4-yl)quinazolin-6-yl)-1-methylazetidine-2-carboxamide and (R)—N-(7-methoxy-4-(1-methyl-3-phenyl-1H-pyrazol-4-yl)quinazolin-6-yl)-1-methylazetidine-2-carboxamide

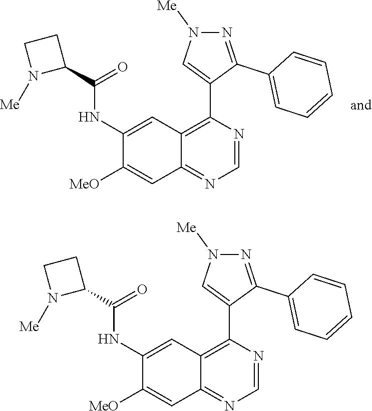 [SMILES: COc1cc2ncnc(-c3cn(C)nc3-c3ccccc3)c2cc1NC(=O)[C@@H]1CCN1C ; COc1cc2ncnc(-c3cn(C)nc3-c3ccccc3)c2cc1NC(=O)[C@H]1CCN1C]

N-(7-methoxy-4-(1-methyl-3-phenyl-1H-pyrazol-4-yl)quinazolin-6-yl)-1-methylazetidine-2-carboxamide (Example 251) was separated by chiral-HPLC (CHIRALPAK IG, 20×250 mm, 5 μm; 40% EtOH/Hex (0.5% 2M NH₃—MeOH)) to afford the title compounds:

Peak 1, white solid (1.4 mg, 14%). Example 252: (S)—N-(7-methoxy-4-(1-methyl-3-phenyl-1H-pyrazol-4-yl)quinazolin-6-yl)-1-methylazetidine-2-carboxamide or (R)—N-(7-methoxy-4-(1-methyl-3-phenyl-1H-pyrazol-4-yl)quinazolin-6-yl)-1-methylazetidine-2-carboxamide. LCMS: m/z=429 [M+H]⁺; ¹H NMR (400 MHZ, DMSO-d₆) δ: 9.93 (s, 1H), 9.06 (s, 1H), 8.87 (s, 1H), 8.20 (s, 1H), 7.50 (s, 1H), 7.30-7.23 (m, 2H), 7.23-7.16 (m, 3H), 4.12 (s, 3H), 4.03 (s, 3H), 3.55 (t, 1H), 3.43-3.34 (m, 1H), 2.98 (q, 1H), 2.34 (s, 3H), 2.40-2.28 (m, 1H), 2.01 (dt, 1H).

Peak 2 as a white solid (1.6 mg, 16%). Example 253: (R)—N-(7-methoxy-4-(1-methyl-3-phenyl-1H-pyrazol-4-yl)quinazolin-6-yl)-1-methylazetidine-2-carboxamide or (S)—N-(7-methoxy-4-(1-methyl-3-phenyl-1H-pyrazol-4-yl)quinazolin-6-yl)-1-methylazetidine-2-carboxamide. LCMS: m/z=429 [M+H]⁺; ¹H NMR (400 MHZ, DMSO-d₆) δ: 9.94 (s, 1H), 9.06 (s, 1H), 8.86 (s, 1H), 8.20 (s, 1H), 7.50 (s, 1H), 7.30-7.23 (m, 2H), 7.23-7.16 (m, 3H), 4.12 (s, 3H), 4.03 (s, 3H), 3.55 (s, 1H), 2.99 (s, 1H), 2.35 (s, 4H), 2.01 (s, 1H).

### Example 254 and 254: (1S,5S)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide and (1R,5R)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide

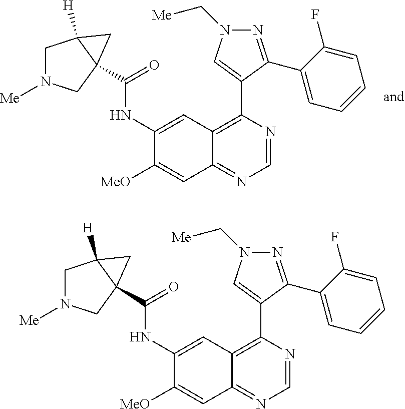 [SMILES: CCn1cc(-c2ncnc3cc(OC)c(NC(=O)[C@@]45C[C@@H]4CN(C)C5)cc23)c(-c2ccccc2F)n1 ; CCn1cc(-c2ncnc3cc(OC)c(NC(=O)[C@]45C[C@H]4CN(C)C5)cc23)c(-c2ccccc2F)n1]

Part 1. To a solution of 4-(3-(2-fluorophenyl)-1-methyl-1H-pyrazol-4-yl)-7-methoxyquinazolin-6-amine (Preparation 253, 150 mg, 0.429 mmol) in DMF (10 mL) was added 3-(tert-butoxycarbonyl)-3-azabicyclo[3.1.0]hexane-1-carboxylic acid (290 mg, 1.28 mmol), DIPEA (332 mg, 2.57 mmol) and HATU (813 mg, 2.14 mmol), was stirred at 60° C. for 3 h. The reaction mixture was diluted with H₂O (20 mL) and extracted with EtOAc (2×20 mL). The combined organics were washed with brine (20 mL), dried (Na₂SO₄) and evaporated to dryness in vacuo. The residue was purified by TLC (10:1 DCM/MeOH) to afford tert-butyl 1-((4-(3-(2-fluorophenyl)-1-methyl-1H-pyrazol-4-yl)-7-methoxyquinazolin-6-yl)carbamoyl)-3-azabicyclo[3.1.0]hexane-3-carboxylate as a white solid (160 mg, 67%). LCMS: m/z=559 [M+H]⁺.

Part 2. tert-butyl 1-((4-(3-(2-fluorophenyl)-1-methyl-1H-pyrazol-4-yl)-7-methoxyquinazolin-6-yl)carbamoyl)-3-azabicyclo[3.1.0]hexane-3-carboxylate (Part 1, 160 mg, 0.286 mmol) in DCM (10 mL) and TFA (3 mL) was stirred at 25° C. for 2 h. The reaction was evaporated to dryness in vacuo to afford N-(4-(3-(2-fluorophenyl)-1-methyl-1H-pyrazol-4-yl)-7-methoxyquinazolin-6-yl)-3-azabicyclo[3.1.0]hexane-1-carboxamide trifluoroacetate as a yellow solid (110 mg, 84%). LCMS: m/z=459 [M+H]⁺.

Part 3. To a solution of N-(4-(3-(2-fluorophenyl)-1-methyl-1H-pyrazol-4-yl)-7-methoxyquinazolin-6-yl)-3-azabicyclo[3.1.0]hexane-1-carboxamide trifluoroacetate (Part 2, 110 mg, 0.239 mmol) in DCM (10 mL) and HCHO (37% in H₂O, 1 mL) was added AcOH (1 mL) and the mixture stirred at 25° C. for 30 min. To this was added NaBH(OAc)₃ (101 mg, 0.478 mmol) and stirring continued for 2 h. The reaction mixture was diluted with EtOAc (100 mL) and washed with brine (2×50 mL). The combined organics were dried (Na₂SO₄) and evaporated to dryness in vacuo to afford N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide as a white solid which was purified by prep-HPLC (Chiralpak AD-H, 20×250 mm, 5 μm; 30% IPA/Hex (0.5% 2M NH₃—MeOH)) to afford the title compounds.

Peak 1, off-white solid (24.2 mg, 22%). Example 254, (1S,5S)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide or (1R,5R)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide. LCMS: m/z=473 [M+H]⁺; ¹H NMR (300 MHz, DMSO-d₆): δ 8.90 (s, 1H), 8.67 (s, 1H), 8.53 (s, 1H), 8.31 (s, 1H), 7.61-7.50 (m, 1H), 7.44 (s, 1H), 7.32 (d, 1H), 7.18 (d, 1H), 6.98 (d, 1H), 4.06 (d, 6H), 3.20 (d, 1H), 2.91 (d, 1H), 2.59 (d, 1H), 2.39-2.26 (m, 4H), 1.95 (d, 1H), 1.31 (d, 1H), 1.27-1.12 (m, 1H).

Peak 2, off-white solid (26.3 mg, 24%). Example 255, (1R,5R)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide or (1S,5S)—N-(4-(3-(2-fluorophenyl)-1-methyl-1H-pyrazol-4-yl)-7-methoxyquinazolin-6-yl)-3-methyl-3-azabicyclo[3.1.0]hexane-1-carboxamide. LCMS: m/z=473 [M+H]⁺; ¹H NMR (300 MHz, DMSO-d₆) δ: 8.90 (s, 1H), 8.67 (s, 1H), 8.53 (s, 1H), 8.31 (s, 1H), 7.57 (t, 1H), 7.44 (s, 1H), 7.31 (s, 1H), 7.19 (s, 1H), 6.98 (d, 1H), 4.06 (d, 6H), 3.20 (d, 1H), 2.92 (s, 1H), 2.58 (s, 1H), 2.38-2.25 (m, 4H), 1.96 (s, 1H), 1.32 (s, 1H), 1.19 (s, 1H).

### Example 256 and 257: (1S,5S)—N-(7-ethoxy-4-(1-methyl-3-phenyl-1H-pyrazol-4-yl)quinazolin-6-yl)-3-methyl-3-azabicyclo[3.1.0]hexane-1-carboxamide and (1R,5R)—N-(7-ethoxy-4-(1-methyl-3-phenyl-1H-pyrazol-4-yl)quinazolin-6-yl)-3-methyl-3-azabicyclo[3.1.0]hexane-1-carboxamide

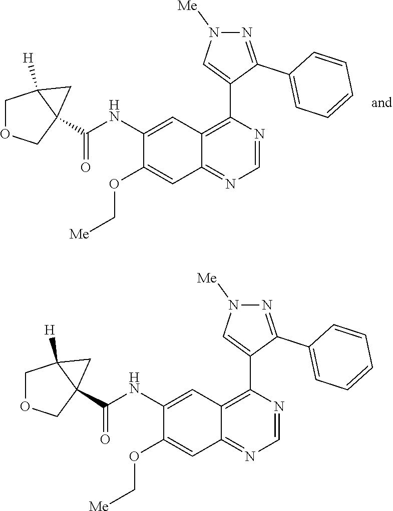 [SMILES: CCOc1cc2ncnc(-c3cn(C)nc3-c3ccccc3)c2cc1NC(=O)[C@]12COC[C@H]1C2 ; CCOc1cc2ncnc(-c3cn(C)nc3-c3ccccc3)c2cc1NC(=O)[C@@]12COC[C@@H]1C2]

Part 1. tert-butyl 1-((7-ethoxy-4-(1-methyl-3-phenyl-1H-pyrazol-4-yl)quinazolin-6-yl)carbamoyl)-3-azabicyclo[3.1.0]hexane-3-carboxylate (90 mg) was prepared from 6-bromo-7-ethoxy-4-(1-methyl-3-phenyl-1H-pyrazol-4-yl)quinazoline (Preparation 90) using an analogous method to that described for Example 306. LCMS: m/z=555 [M+H]⁺.

Part 2. tert-butyl 1-((7-ethoxy-4-(1-methyl-3-phenyl-1H-pyrazol-4-yl)quinazolin-6-yl)carbamoyl)-3-azabicyclo[3.1.0]hexane-3-carboxylate (Part 1, 90 mg, 0.162 mmol) was added to an ice-cold mixture of 1:4 DCM/(HCl in dioxane) and the resulting mixture stirred at rt for 1 h and then evaporated to dryness in vacuo to afford a white solid. The solid was dissolved in 10:1 MeOH/AcOH and cooled to 0° C. before HCHO (1 mL) and sodium cyanoborohydride (24.9 mg, 0.396 mmol) added. The resulting mixture was stirred at rt for 3 h. The mixture was diluted with EtOAc (120 mL) and washed with water (60 mL). The organics were dried (Na₂SO₄) and evaporated to dryness in vacuo. The residue was purified by prep-HPLC (YMC-Actus Triart C18 ExRS, 30×150 mm, 5 μm; 30-47% MeCN/H₂O (10 mM NH₄HCO₃+0.1% NH₄OH)) to afford N-(7-ethoxy-4-(1-methyl-3-phenyl-1H-pyrazol-4-yl)quinazolin-6-yl)-3-methyl-3-azabicyclo[3.1.0]hexane-1-carboxamide as an off-white solid (50 mg). LCMS: m/z=469 [M+H]⁺.

Part 3. N-(7-ethoxy-4-(1-methyl-3-phenyl-1H-pyrazol-4-yl)quinazolin-6-yl)-3-methyl-3-azabicyclo[3.1.0]hexane-1-carboxamide (Part 2) was further separated by chiral-SFC (CHIRAL ART Amylose-C NEO, 30×250 mm, 5 μm; Mobile 35% IPA (0.5% 2M NH₃—MeOH) in CO₂) to afford the title compounds.

Peak 1, off-white solid (10.9 mg). Example 256, (1S,5S)—N-(7-ethoxy-4-(1-methyl-3-phenyl-1H-pyrazol-4-yl)quinazolin-6-yl)-3-methyl-3-azabicyclo[3.1.0]hexane-1-carboxamide or (1R,5R)—N-(7-ethoxy-4-(1-methyl-3-phenyl-1H-pyrazol-4-yl)quinazolin-6-yl)-3-methyl-3-azabicyclo[3.1.0]hexane-1-carboxamide. LCMS: m/z=469 [M+H]⁺; ¹H NMR (400 MHZ, DMSO-d₆) δ: 9.04 (s, 1H), 8.54 (s, 1H), 8.47 (s, 1H), 8.16 (s, 1H), 7.44 (s, 1H), 7.30-7.23 (m, 2H), 7.23-7.16 (m, 3H), 4.34 (q, 2H), 4.02 (s, 3H), 3.17 (d, 1H), 2.90 (d, 1H), 2.56 (d, 1H), 2.27 (s, 4H), 1.96-1.89 (m, 1H), 1.47 (t, 3H), 1.31 (t, 1H), 1.23 (s, 1H), 1.16 (dd, 1H).

Peak 2, off-white solid (12.8 mg). Example 257, (1R,5R)—N-(7-ethoxy-4-(1-methyl-3-phenyl-1H-pyrazol-4-yl)quinazolin-6-yl)-3-methyl-3-azabicyclo[3.1.0]hexane-1-carboxamide or (1S,5S)—N-(7-ethoxy-4-(1-methyl-3-phenyl-1H-pyrazol-4-yl)quinazolin-6-yl)-3-methyl-3-azabicyclo[3.1.0]hexane-1-carboxamide. LCMS: m/z=469 [M+H]⁺; ¹H NMR (400 MHZ, DMSO-d₆) δ: 9.04 (s, 1H), 8.53 (s, 2H), 8.16 (s, 1H), 7.44 (s, 1H), 7.30-7.23 (m, 2H), 7.23-7.15 (m, 3H), 4.34 (q, 2H), 4.02 (s, 3H), 3.19 (s, 1H), 2.92 (s, 1H), 2.60 (s, 1H), 2.29 (s, 4H), 1.94 (s, 1H), 1.47 (t, 3H), 1.31 (t, 1H), 1.23 (s, 1H), 1.17 (s, 1H).

### Example 258 and 259: (S)—N-(7-methoxy-4-(1-methyl-3-phenyl-1H-pyrazol-4-yl)quinazolin-6-yl)-2-methyltetrahydrofuran-2-carboxamide and (R)—N-(7-methoxy-4-(1-methyl-3-phenyl-1H-pyrazol-4-yl)quinazolin-6-yl)-2-methyltetrahydrofuran-2-carboxamide

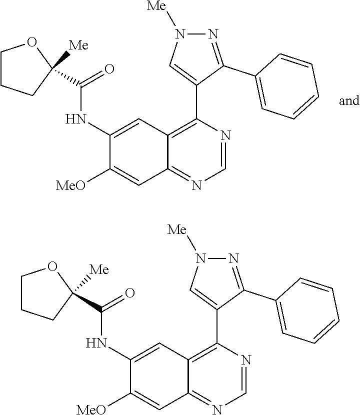

N-(7-methoxy-4-(1-methyl-3-phenyl-1H-pyrazol-4-yl)quinazolin-6-yl)-2-methyltetrahydrofuran-2-carboxamide (Example 224, 21 mg, 47.3 μmol) was purified by prep-HPLC (Chiralpak AD-H, 20×250 mm, 5 μm; 50% EtOH/Hex (0.5% 2M NH₃—MeOH)) to afford the title compounds.

Peak 1, off-white solid (2.5 mg). Example 258, (S)—N-(7-methoxy-4-(1-methyl-3-phenyl-1H-pyrazol-4-yl)quinazolin-6-yl)-2-methyltetrahydrofuran-2-carboxamide or (R)—N-(7-methoxy-4-(1-methyl-3-phenyl-1H-pyrazol-4-yl)quinazolin-6-yl)-2-methyltetrahydrofuran-2-carboxamide. LCMS: m/z=444 [M+H]⁺; ¹H NMR (400 MHZ, DMSO-d₆) δ: 9.37 (s, 1H), 9.07 (s, 1H), 8.80 (s, 1H), 8.23 (s, 1H), 7.48 (s, 1H), 7.29-7.19 (m, 5H), 4.04 (s, 6H), 4.02-3.86 (m, 2H), 2.21 (ddd, 1H), 1.99-1.88 (m, 3H), 1.37 (s, 3H).

Peak 2, off-white solid (1.9 mg). Example 259, (R)—N-(7-methoxy-4-(1-methyl-3-phenyl-1H-pyrazol-4-yl)quinazolin-6-yl)-2-methyltetrahydrofuran-2-carboxamide or (S)—N-(7-methoxy-4-(1-methyl-3-phenyl-1H-pyrazol-4-yl)quinazolin-6-yl)-2-methyltetrahydrofuran-2-carboxamide. LCMS: m/z=444 [M+H]⁺; ¹H NMR (400 MHZ, DMSO-d₆) δ: 9.37 (s, 1H), 9.07 (s, 1H), 8.80 (s, 1H), 8.23 (s, 1H), 7.48 (s, 1H), 7.23-7.12 (m, 5H), 4.10 (s, 6H), 4.02-3.86 (m, 2H), 2.26-2.14 (m, 1H), 2.00-1.88 (m, 3H), 1.37 (s, 3H).

### Example 260 and 261: (1S,5S)—N-(7-methoxy-4-(1-methyl-3-phenyl-1H-pyrazol-4-yl)quinazolin-6-yl)-5-methyl-3-oxabicyclo[3.1.0]hexane-1-carboxamide and (1R,5R)—N-(7-methoxy-4-(1-methyl-3-phenyl-1H-pyrazol-4-yl)quinazolin-6-yl)-3-oxabicyclo[3.1.0]hexane-1-carboxamide

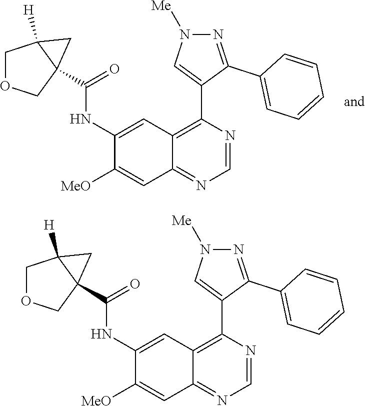

Part 1. N-(7-methoxy-4-(1-methyl-3-phenyl-1H-pyrazol-4-yl)quinazolin-6-yl)-3-oxabicyclo[3.1.0]hexane-1-carboxamide was prepared (50 mg, 23%) from 6-bromo-7-methoxy-4-(1-methyl-3-phenyl-1H-pyrazol-4-yl)quinazoline (Preparation 91) and 3-oxabicyclo[3.1.0]hexane-1-carboxamide (Preparation 64) using an analogous method to that described for Example 306. LCMS: m/z=442 [M+H]⁺.

Part 2. N-(7-methoxy-4-(1-methyl-3-phenyl-1H-pyrazol-4-yl)quinazolin-6-yl)-3-oxabicyclo[3.1.0]hexane-1-carboxamide (Part 1) was further separated by chiral-HPLC (CHIRAL ART Cellulose-SB, 20×250 mm, 5 μm; 10% EtOH/MTBE (0.5% 2M NH₃—MeOH)) to afford the title compounds.

Peak 1, white solid (18.7 mg). Example 260, (1S,5S)—N-(7-methoxy-4-(1-methyl-3-phenyl-1H-pyrazol-4-yl)quinazolin-6-yl)-5-methyl-3-oxabicyclo[3.1.0]hexane-1-carboxamide or (1R,5R)—N-(7-methoxy-4-(1-methyl-3-phenyl-1H-pyrazol-4-yl)quinazolin-6-yl)-3-oxabicyclo[3.1.0]hexane-1-carboxamide. LCMS: m/z=442 [M+H]⁺; ¹H NMR (300 MHz, DMSO-d₆) δ: 9.06 (s, 1H), 8.69 (s, 1H), 8.43 (s, 1H), 8.17 (s, 1H), 7.46 (s, 1H), 7.27 (ddt, 2H), 7.20 (dt, 3H), 4.04 (d, 6H), 3.95 (s, 2H), 3.79-3.66 (m, 2H), 2.22-2.11 (m, 1H), 1.39 (dd, 1H), 0.87 (t, 1H).

Peak 2, white solid (18.0 mg). Example 261, (1R,5R)—N-(7-methoxy-4-(1-methyl-3-phenyl-1H-pyrazol-4-yl)quinazolin-6-yl)-5-methyl-3-oxabicyclo[3.1.0]hexane-1-carboxamide or (1S,5S)—N-(7-methoxy-4-(1-methyl-3-phenyl-1H-pyrazol-4-yl)quinazolin-6-yl)-3-oxabicyclo[3.1.0]hexane-1-carboxamide. LCMS: m/z=442 [M+H]⁺; ¹H NMR (300 MHz, DMSO-d₆) δ: 9.06 (s, 1H), 8.69 (s, 1H), 8.43 (s, 1H), 8.17 (s, 1H), 7.46 (s, 1H), 7.27 (ddt, 2H), 7.20 (dt, 3H), 4.04 (d, 6H), 3.95 (s, 2H), 3.79-3.66 (m, 2H), 2.22-2.11 (m, 1H), 1.39 (dd, 1H), 0.87 (t, 1H).

### Example 262 and 263: (1R,5R)—N-(4-(1-ethyl-3-(2-fluorophenyl)-1H-pyrazol-4-yl)-7-methoxyquinazolin-6-yl)-3-oxabicyclo[3.1.0]hexane-1-carboxamide and (1S,5S)—N-(4-(1-ethyl-3-(2-fluorophenyl)-1H-pyrazol-4-yl)-7-methoxyquinazolin-6-yl)-3-oxabicyclo[3.1.0]hexane-1-carboxamide

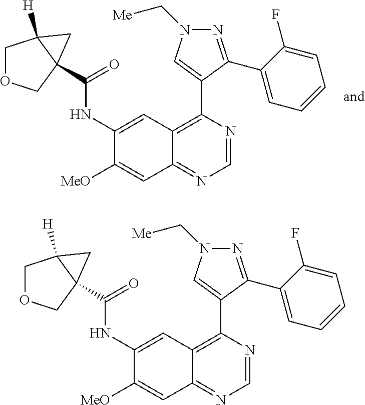 [SMILES: CCn1cc(-c2ncnc3cc(OC)c(NC(=O)[C@@]45COC[C@@H]4C5)cc23)c(-c2ccccc2F)n1 ; CCn1cc(-c2ncnc3cc(OC)c(NC(=O)[C@]45COC[C@H]4C5)cc23)c(-c2ccccc2F)n1]

Part 1. Pd₂(dba)₃ (42.8 mg, 0.047 mmol), XPhos (27.0 mg, 0.047 mmol) and K₂CO₃ (96.8 mg, 0.702 mmol) were added to a solution of 6-bromo-4-(1-ethyl-3-(2-fluorophenyl)-1H-pyrazol-4-yl)-7-methoxyquinazoline (Preparation 98, 200 mg, 0.468 mmol) and 3-oxabicyclo[3.1.0]hexane-1-carboxamide (89.2 mg, 0.702 mmol) in toluene and the mixture stirred at 100° C. for 3 h. The reaction mixture was extracted with EtOAc (3×50 mL) and the combined organics dried (Na₂SO₄) and evaporated to dryness in vacuo. The residue was purified by prep-HPLC (YMC-Actus Triart C18 ExRS, 30×150 mm, 5 μm; 30-53% MeCN/H₂O (10 mM NH₄HCO₃+0.1% NH₄OH)) to afford N-(4-(1-ethyl-3-(2-fluorophenyl)-1H-pyrazol-4-yl)-7-methoxyquinazolin-6-yl)-3-oxabicyclo[3.1.0]hexane-1-carboxamide as a white solid (100 mg).

Part 2. N-(4-(1-ethyl-3-(2-fluorophenyl)-1H-pyrazol-4-yl)-7-methoxyquinazolin-6-yl)-3-oxabicyclo[3.1.0]hexane-1-carboxamide (Part 1, 100 mg) was further purified by prep-SFC (Chiralpak IG, 30×250 mm, 5 μm; 35% MeOH (0.1% 2 M NH₃—MeOH) in CO₂) to afford the title compounds:

Peak 1, Example 262, yellow solid, 34.5 mg: (1R,5R)—N-(4-(1-ethyl-3-(2-fluorophenyl)-1H-pyrazol-4-yl)-7-methoxyquinazolin-6-yl)-3-oxabicyclo[3.1.0]hexane-1-carboxamide and (1S,5S)—N-(4-(1-ethyl-3-(2-fluorophenyl)-1H-pyrazol-4-yl)-7-methoxyquinazolin-6-yl)-3-oxabicyclo[3.1.0]hexane-1-carboxamide. LCMS: m/z=474 [M+H]⁺; ¹H NMR (400 MHZ, DMSO-d₆) δ: 8.91 (s, 1H), 8.70 (s, 1H), 8.59 (s, 1H), 8.36 (s, 1H), 7.59 (td, 1H), 7.43 (s, 1H), 7.36-7.30 (m, 1H), 7.20 (dt, 1H), 6.99-6.94 (m, 1H), 4.33 (q, 2H), 4.05 (s, 3H), 3.97 (s, 2H), 3.77-3.74 (m, 2H), 2.21-2.17 (m, 1H), 1.53 (t, 3H), 1.43-1.39 (m, 2H).

Peak 2, Example 263, brown solid, 45.1 mg: (1S,5S)—N-(4-(1-ethyl-3-(2-fluorophenyl)-1H-pyrazol-4-yl)-7-methoxyquinazolin-6-yl)-3-oxabicyclo[3.1.0]hexane-1-carboxamide and (1R,5R)—N-(4-(1-ethyl-3-(2-fluorophenyl)-1H-pyrazol-4-yl)-7-methoxyquinazolin-6-yl)-3-oxabicyclo[3.1.0]hexane-1-carboxamide. LCMS: m/z=474 [M+H]⁺; ¹H NMR (400 MHZ, DMSO-d₆) δ: ppm 8.91 (s, 1H), 8.70 (s, 1H), 8.59 (s, 1H), 8.36 (s, 1H), 7.59 (td, 1H), 43 (s, 1H), 7.36-7.30 (m, 1H), 7.20 (dt, 1H), 6.99-6.94 (m, 1H), 4.34 (q, 2H), 4.05 (s, 3H), 3.97 (s, 2H), 3.77-3.74 (m, 2H), 2.21-2.17 (m, 1H), 1.53 (t, 3H), 1.43-1.39 (m, 2H).

### Example 264:2-fluoro-N-(4-(1-(2-hydroxyethyl)-3-phenyl-1H-pyrazol-4-yl)-7-methoxyquinazolin-6-yl)benzamide

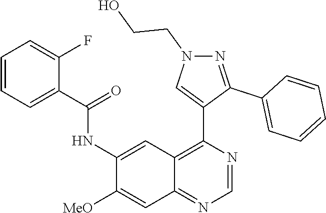 [SMILES: COc1cc2ncnc(-c3cn(CCO)nc3-c3ccccc3)c2cc1NC(=O)c1ccccc1F]

A solution of N-(4-(1-(2-((tert-butyldimethylsilyl)oxy)ethyl)-3-phenyl-1H-pyrazol-4-yl)-7-methoxyquinazolin-6-yl)-2-fluorobenzamide (Preparation 207, 80 mg, 116 μmol) and 7M HCl in dioxane (10 mL) was stirred at 25° C. for 2 h. The reaction mixture was evaporated to dryness and the residue purified by prep-HPLC (XBridge Prep OBD C18 Column, 30×150 mm, 5 μm; 42-72% MeCN/H₂O (10 mM NH₄HCO₃+0.1% NH₄OH)) to afford the title compound as an off-white solid (20.3 mg, 35%). LCMS: m/z=484 [M+H]⁺; ¹H NMR (300 MHz, DMSO-d₆) δ: 9.74 (d, 1H), 9.09 (s, 1H), 8.87 (s, 1H), 8.25 (s, 1H), 7.83 (t, 1H), 7.69-7.58 (m, 1H), 7.51 (s, 1H), 7.45-7.27 (m, 4H), 7.21 (q, 3H), 5.06 (t, 1H), 4.35 (t, 2H), 4.10 (s, 3H), 3.90 (m, 2H).

### Example 265: N-(4-(1-(2-hydroxyethyl)-3-phenyl-1H-pyrazol-4-yl)-7-methoxyquinazolin-6-yl)bicyclo[1.1.1]pentane-1-carboxamide

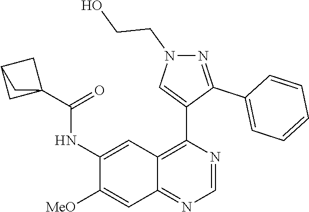 [SMILES: COc1cc2ncnc(-c3cn(CCO)nc3-c3ccccc3)c2cc1NC(=O)C12CC(C1)C2]

The title compound was prepared as a white solid (58 mg, 52%) from N-(4-(1-(2-((tert-butyldimethyl silyl)oxy)ethyl)-3-phenyl-1H-pyrazol-4-yl)-7-methoxyquinazolin-6-yl)bicyclo[1.1.1]pentane-1-carboxamide (Preparation 208) using an analogous method to that described for Example 264. YMC-Actus Triart C18, 30×150 mm, 5 μm; 35-50% MeCN/H₂O (10 mM NH₄HCO₃+0.1% NH₄OH)). LCMS: m/z=456 [M+H]⁺; ¹H NMR (300 MHz, DMSO-d₆) δ: 9.06 (s, 1H), 8.71 (s, 1H), 8.55 (s, 1H), 8.19 (s, 1H), 7.47 (s, 1H), 7.34-7.25 (m, 2H), 7.21 (q, 3H), 5.04 (t, 1H), 4.33 (t, 2H), 4.07 (s, 3H), 3.88 (q, 2H), 2.47 (s, 1H), 2.08 (s, 6H).

### Example 266: N-(7-methoxy-4-(2-methyl-4-phenylthiazol-5-yl)quinazolin-6-yl)cyclopropanecarboxamide

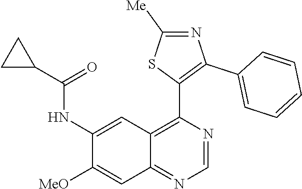 [SMILES: COc1cc2ncnc(-c3sc(C)nc3-c3ccccc3)c2cc1NC(=O)C1CC1]

Pd₂(dppf)₃ (36.3 mg, 49.6 μmol) and K₂CO₃ (102 mg, 0.745 mmol) were added to 2-methyl-4-phenyl-5-(4,4,5,5-tetramethyl-1,3,2-dioxaborolan-2-yl)-1,3-thiazole (150 mg, 0.497 mmol) and N-(4-chloro-7-methoxyquinazolin-6-yl)cyclopropanecarboxamide (Preparation 201, 137 mg, 0.496 mmol) in H₂O (1.5 mL) and dioxane (6 mL) at rt and the resulting was heated at 80° C. for 2 h under N₂. The reaction mixture was extracted with EtOAc (3×50 mL) and the combined organics dried (Na₂SO₄) and evaporated to dryness in vacuo. The residue was purified by prep-HPLC (XBridge Shield RP18 OBD Column, 30×150 mm, 5 μm; 13-61% MeCN/H₂O (10 mM NH₄HCO₃+0.1% NH₄OH)) to afford the title compound was a white solid (87 mg, 42%). LCMS: m/z=417 [M+H]⁺; ¹H NMR (400 MHZ, DMSO-d₆) δ: 9.65 (s, 1H), 9.14 (s, 1H), 8.54 (d, 1H), 7.44 (s, 1H), 7.25-7.20 (m, 2H), 7.19-7.11 (m, 3H), 4.06 (s, 3H), 2.82 (s, 3H), 2.13 (p, 1H), 0.76 (d, 4H).

### Example 267:4-(7-ethoxy-4-(1-methyl-3-phenyl-1H-pyrazol-4-yl)quinazolin-6-yl)morpholin-3-one

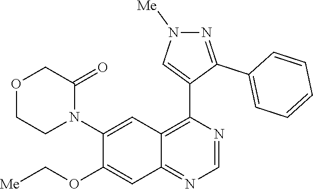 [SMILES: CCOc1cc2ncnc(-c3cn(C)nc3-c3ccccc3)c2cc1N1CCOCC1=O]

A mixture of 6-bromo-7-ethoxy-4-(1-methyl-3-phenyl-1H-pyrazol-4-yl)quinazoline (Preparation 90, 200 mg, 0.488 mol), morpholin-3-one (49.3 mg, 0.488 mmol), CuI (9.61 mg, 0.0488 mmol), N1,N2-dimethylethane-1,2-diamine (8.58 mg, 0.0976 mmol), K₃PO₄ (206 mg, 0.976 mmol) in dioxane (5 mL) was stirred 120° C. for 10 h under N₂. The solids were removed by filtration and the filtrate evaporated to dryness. The residue was purified by prep-HPLC (XBridge Shield RP18 OBD Column, 30×150 mm, 5 μm; 47% MeCN/H₂O (10 mM NH₄HCO₃+0.1% NH₄OH)) to afford the title compound as a white solid (8.3 mg). LCMS: m/z=430 [M+H]⁺; ¹H NMR (300 MHZ, DMSO-d₆) δ: 9.14 (s, 1H), 8.25 (s, 1H), 7.66 (s, 1H), 7.47 (s, 1H), 7.32-7.18 (m, 6H), 4.25 (m, 2H), 4.15 (s, 2H), 4.02 (s, 3H), 3.88 (m, 2H), 3.24 (m, 2H), 1.36 (t, 3H).

### Example 268: N-(4-(1-(2,2-difluoroethyl)-3-phenyl-1H-pyrazol-4-yl)-7-methoxyquinazolin-6-yl)-1-ethyl-1H-pyrazole-4-carboxamide

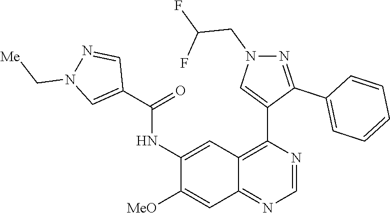 [SMILES: CCn1cc(C(=O)Nc2cc3c(-c4cn(CC(F)F)nc4-c4ccccc4)ncnc3cc2OC)cn1]

To the solution of 4-(1-(2,2-difluoroethyl)-3-phenyl-1H-pyrazol-4-yl)-7-methoxyquinazolin-6-amine (Preparation 251, 50 mg, 0.131 mmol) and 1-ethyl-1H-pyrazole-4-carboxylic acid (27.6 mg, 0.197 mmol) in pyridine (2 mL) was added EDCl (37.7 mg, 0.197 mol) and the mixture stirred at 60° C. for 4 h. The reaction mixture was evaporated to dryness and the residue purified by prep-HPLC (Waters Xbridge BEH C18, 30×100 mm, 10 μm; 20-45% MeCN/H₂O (10 mM NH₄HCO₃+0.05% NH₄OH)) to afford the title compound as a white solid (14.9 mg, 21%). LCMS: m/z=504 [M+H]⁺; ¹H NMR (400 MHZ, CDCl₃) δ: 9.12 (s, 1H), 9.04 (s, 1H), 8.28 (s, 1H), 8.01 (s, 1H), 7.97 (s, 1H), 7.85 (s, 1H), 7.41-7.46 (m, 2H), 7.40 (s, 1H), 7.22 (d, 2H), 7.21 (d, 1H), 6.12-6.47 (m, 1H), 4.66 (td, 2H), 4.25 (q, 2H), 4.14 (s, 3H), 1.55 (t, 3H).

### Example 269-274

The title compounds were prepared from 4-(1-(2,2-difluoroethyl)-3-phenyl-1H-pyrazol-4-yl)-7-methoxyquinazolin-6-amine (Preparation 251) and the appropriate carboxylic acid (RCO₂H) using an analogous method to that described for Example 268.

| Example | |
|---|---|
| Number | Name/Structure/Amine/RCO₂H/Data |
| 269 | N-(4-(1-(2,2-difluoroethyl)-3-phenyl-1H-pyrazol-4-yl)-7-methoxyquinazolin-6-yl)- |
| | 2,2-difluoroacetamide |
| | 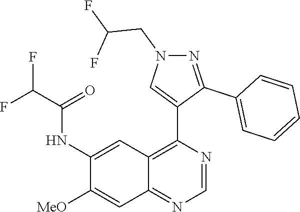 [SMILES: COc1cc2ncnc(-c3cn(CC(F)F)nc3-c3ccccc3)c2cc1NC(=O)C(F)F] |
| | Acid: 2,2-difluoroacetic acid |
| | HPLC: XBridge BEH C18, 30 x 150 mm, 10 μm; 20-50% MeCN/H₂O (10 mM |
| | NH₄HCO₃ + 0.05% NH₄OH) |
| | White solid (4.4 mg, 6.7%). LCMS: m/z = 460 [M + H]⁺; ¹H NMR (400 MHz, |
| | CDCl₃) δ: 9.10 (s, 1H), 8.80 (s, 1H), 8.62 (s, 1H), 7.91 (s, 1H), 7.33 (s, 2H), 7.30 |
| | (d, 1H), 7.16-7.11 (m, 3H), 6.38-6.06 (m, 1H), 6.06-5.77 (m, 1H), 4.56 (td, 2H), |
| | 4.04 (s, 3H). |
| 270 | N-(4-(1-(2,2-difluoroethyl)-3-phenyl-1H-pyrazol-4-yl)-7-methoxyquinazolin-6-yl)- |
| | 1-isopropyl-1H-pyrazole-4-carboxamide |
| | 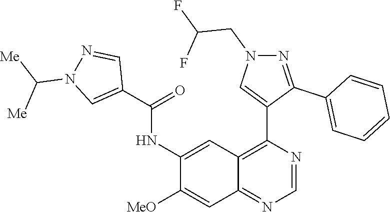 [SMILES: COc1cc2ncnc(-c3cn(CC(F)F)nc3-c3ccccc3)c2cc1NC(=O)c1cnn(C(C)C)c1] |
| | Acid: 1-isopropyl-1H-pyrazole-4-carboxylic acid |
| | HPLC: Phenomenex Gemini-NX C18, 30 x 75 mm, 3 μm; 35-60% MeCN/H₂O (10 |
| | mM NH₄HCO₃ + 0.05% NH₄OH) |
| | Yellow solid (36.3 mg, 52%). LCMS: m/z = 518 [M + H]⁺; ¹H NMR (400 MHz, |
| | CDCl₃) δ: 9.12 (s, 1H), 9.04 (s, 1H), 8.28 (s, 1H), 8.00 (d, 2H), 7.85 (s, 1H), 7.44 |
| | (dd, 2H), 7.40 (s, 1H), 7.24-7.20 (m, 3H), 6.48-6.10 (m, 1H), 4.65 (td, 2H), 4.56 |
| | (dt, 1H), 4.14 (s, 3H), 1.56 (d, 6H). |
| 271 | N-(4-(1-(2,2-difluoroethyl)-3-phenyl-1H-pyrazol-4-yl)-7-methoxyquinazolin-6- |
| | yl)isothiazole-4-carboxamide |
| | 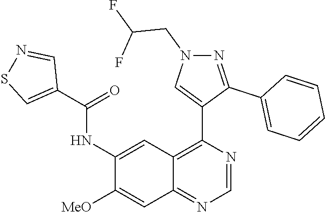 [SMILES: COc1cc2ncnc(-c3cn(CC(F)F)nc3-c3ccccc3)c2cc1NC(=O)c1cnsc1] |
| | Acid: isothiazole-4-carboxylic acid |
| | HPLC: Phenomenex Gemini-NX C18, 30 x 75 mm, 3 μm; 25-55% MeCN/H₂O (10 |
| | mM NH₄HCO₃ + 0.05% NH₄OH) |
| | Yellow solid (75 mg, 83%). LCMS: m/z = 493 [M + H]⁺; ¹H NMR (400 MHz, |
| | CDCl₃) δ: 9.21 (s, 1H), 9.15 (s, 1H), 9.03 (s, 1H), 8.90 (s, 1H), 8.46 (s, 1H,), 8.02 |
| | (s, 1H), 7.44 (s, 1H), 7.43-7.41 (m, 2H), 7.22-7.20 (m, 3H), 6.45-6.29 (m, 1H), 4.66 |
| | (td, 2H, J = 17.6, 4.0 Hz), 4.14 (s, 3H). |
| 272 | N-(4-(1-(2,2-difluoroethyl)-3-phenyl-1H-pyrazol-4-yl)-7-methoxyquinazolin-6- |
| | yl)isothiazole-5-carboxamide |
| | 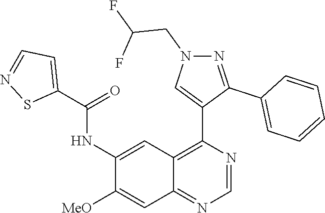 [SMILES: COc1cc2ncnc(-c3cn(CC(F)F)nc3-c3ccccc3)c2cc1NC(=O)c1ccns1] |
| | Acid: isothiazole-5-carboxylic acid |
| | HPLC: Phenomenex Gemini-NX C18, 30 x 75 mm, 3 μm; 30-60% MeCN/H₂O (10 |
| | mM NH₄HCO₃ + 0.05% NH₄OH) |
| | White solid (25.3 mg, 65%). LCMS: m/z = 493 [M + H]⁺; ¹H NMR (400 MHz, |
| | CDCl₃) δ: 9.18 (s, 1H), 9.00 (s, 1H), 8.59 (d, 1H), 8.52 (s, 1H), 8.03 (s, 1H), 7.62 |
| | (d, 1H), 7.49-7.39 (m, 3H), 7.25-7.17 (m, 3H), 6.47-6.15 (m, 1H), 4.67 (td, 2H), |
| | 4.15 (s, 3H). |
| 273 | N-(4-(1-(2,2-difluoroethyl)-3-phenyl-1H-pyrazol-4-yl)-7-methoxyquinazolin-6-yl)- |
| | 4-methylthiazole-5-carboxamide |
| | 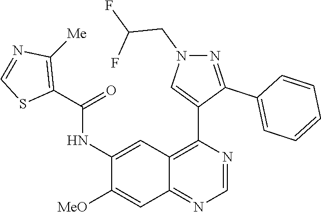 [SMILES: COc1cc2ncnc(-c3cn(CC(F)F)nc3-c3ccccc3)c2cc1NC(=O)c1scnc1C] |
| | Acid: 4-methylthiazole-5-carboxylic acid |
| | HPLC: Phenomenex Gemini-NX C18, 30 x 75 mm, 3 μm; 20-50% MeCN/H₂O (10 |
| | mM NH₄HCO₃ + 0.05% NH₄OH |
| | White solid (16.7 mg, 42%). LCMS: m/z = 507 [M + H]⁺; ¹H NMR (400 MHz, |
| | CDCl₃) δ: 9.07 (s, 1H), 8.90 (s, 1H), 8.35 (s, 1H), 8.03 (s, 1H), 7.95 (s, 1H), 7.39 |
| | (s, 1H), 7.34-7.32 (m, 2H), 7.15-7.11 (m, 3H), 6.36-6.06 (m, 1H), 4.58 (dt, 2H), |
| | 4.08 (s, 3H), 2.72 (s, 3H). |
| 274 | N-(4-(1-(2,2-difluoroethyl)-3-phenyl-1H-pyrazol-4-yl)-7-methoxyquinazolin-6-yl)- |
| | 2-methylthiazole-5-carboxamide |
| | 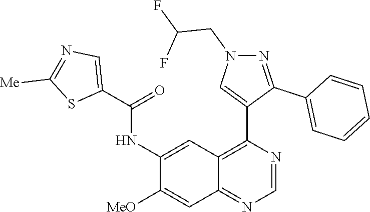 [SMILES: COc1cc2ncnc(-c3cn(CC(F)F)nc3-c3ccccc3)c2cc1NC(=O)c1cnc(C)s1] |
| | Acid: 2-methylthiazole-5-carboxylic acid |
| | HPLC: Phenomenex Gemini-NX C18, 30 x 75 mm, 3 μm; 30-60% MeCN/H₂O (10 |
| | mM NH₄HCO₃ + 0.05% NH₄OH |
| | White solid (11.2 mg, 28%). LCMS: m/z = 507 [M + H]⁺; ¹H NMR (400 MHz, |
| | CDCl₃) δ: 9.07 (s, 1H), 8.90 (s, 1H), 8.35 (s, 1H), 8.03 (s, 1H), 7.95 (s, 1H), 7.39 |
| | (s, 1H), 7.35-7.32 (m, 2H), 7.15-7.11 (m, 3H), 6.37-6.07 (m, 1H), 4.58 (dt, 2H), |
| | 4.08 (s, 3H), 2.72 (s, 3H). |

### Example 275 and 276: (1S,5S)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide and (1R,5R)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide

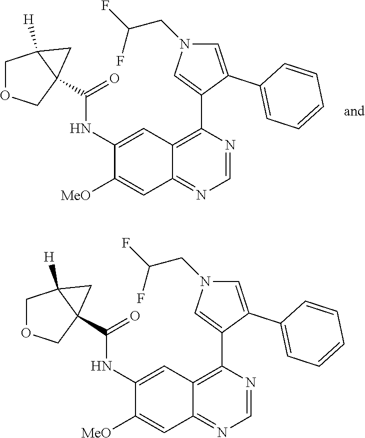 [SMILES: COc1cc2ncnc(-c3cn(CC(F)F)cc3-c3ccccc3)c2cc1NC(=O)[C@]12COC[C@H]1C2 ; COc1cc2ncnc(-c3cn(CC(F)F)cc3-c3ccccc3)c2cc1NC(=O)[C@@]12COC[C@@H]1C2]

To a solution of 4-(1-(2,2-difluoroethyl)-3-phenyl-1H-pyrazol-4-yl)-7-methoxyquinazolin-6-amine (Preparation 251, 238 mg, 0.624 mmol) and 3-oxabicyclo[3.1.0]hexane-1-carboxylic acid (100 mg, 0.780 mmol) in EtOAc (1 mL) was added DIPEA (303 mg, 2.34 mmol) HATU (594 mg, 1.56 mmol) and DMAP (95.4 mg, 0.780 mmol) and the mixture stirred at 70° C. for 12 h. The reaction mixture was filtered and the filtrate concentrated under reduced pressure and the residue was purified by prep-HPLC (Xtimate C18 150*25 mm*5 μm; 20-60% MeCN/H₂O (+0.2% HCO₂H)) to afford N-(4-(1-(2,2-difluoroethyl)-3-phenyl-1H-pyrazol-4-yl)-7-methoxyquinazolin-6-yl)-3-oxabicyclo[3.1.0]hexane-1-carboxamide as a white solid (30 mg, 7.8%) followed by chiral-SFC (Chiralpak IG, 30×250 mm, 10 μm; 40-60% IPA in CO₂) to afford the title compounds.

Peak 1, Example 275, (14.4 mg, 47%): (1S,5S)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide or (1R,5R)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide. LCMS: m/z=492 [M+H]⁺; ¹H NMR (400 MHZ, CDCl₃) δ: 9.12 (1H, s), 8.93 (1H, s), 7.99 (1H, s), 7.90 (1H, s), 7.42-7.39 (2H, m), 7.25-7.19 (3H, m), 6.43-6.12 (1H, m), 4.64 (td, 2H), 4.11 (3H, s), 4.10-4.03 (2H, m), 3.92-3.83 (2H, m), 2.17 (ddd, 1H), 1.49 (dd, 1H), 1.10 (t, 1H).

Peak 2, Example 276, (9.3 mg, 30%): (1R,5R)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide or (1S,5S)—N-(4-(1-(2,2-difluoroethyl)-3-phenyl-1H-pyrazol-4-yl)-7-methoxyquinazolin-6-yl)-3-oxabicyclo[3.1.0]hexane-1-carboxamide. LCMS: m/z=492 [M+H]⁺; ¹H NMR (400 MHZ, CDCl₃) δ: 9.12 (1H, s), 8.93 (1H, s), 7.99 (1H, s), 7.90 (1H, s), 7.42-7.39 (2H, m), 7.25-7.19 (3H, m), 6.43-6.12 (1H, m), 4.64 (td, 2H), 4.11 (3H, s), 4.10-4.03 (2H, m), 3.92-3.83 (2H, m), 2.17 (ddd, 1H), 1.49 (dd, 1H), 1.10 (t, 1H).

### Example 277: N-(4-(1-methyl-3-phenyl-1H-pyrazol-4-yl)-7-(oxetan-3-ylmethoxy) quinazolin-6-yl)propionamide

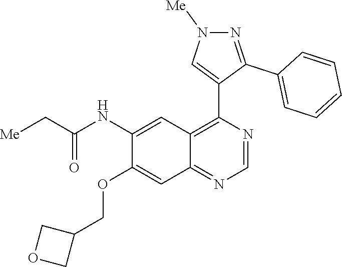 [SMILES: CCC(=O)Nc1cc2c(-c3cn(C)nc3-c3ccccc3)ncnc2cc1OCC1COC1]

To a solution of N-(7-hydroxy-4-(1-methyl-3-phenyl-1H-pyrazol-4-yl)quinazolin-6-yl)propionamide (Preparation 249, 35 mg, 0.094 mmol) and 3-(bromomethyl) oxetane (14.2 mg, 0.094 mmol) in DMF (0.5 mL) was added K₂CO₃ (38.9 mg, 0.281 mmol) and the mixture stirred at 60° C. for 12 h. The reaction mixture was filtered and the filtrate was concentrated in vacuo. The residue was purified by prep-HPLC (Xbridge BEH C18 25×100 mm, 5 μm; 15-45% MeCN/H₂O (10 mM NH4HCO3)-to afford the title compound as a yellow solid (10.7 mg, 26%). LCMS: m/z=441 [M+H]⁺; ¹H NMR (400 MHZ, CDCl₃) δ: 8.12 (s, 1H), 7.90 (s, 1H), 7.45-7.43 (m, 2H), 7.40 (s, 1H), 7.24 (d, 2H), 7.22 (s, 1H), 5.05 (dd, 2H), 4.69 (t, 2H), 4.48 (d, 2H), 4.12 (s, 3H), 3.58-3.51 (m, 1H), 2.49-2.43 (m, 2H), 1.28 (t, 3H).

### Example 278: N-(7-methoxy-4-(1-methyl-3-phenyl-1H-pyrazol-4-yl)quinazolin-6-yl)-3-oxabicyclo[3.1.0]hexane-1-carboxamide

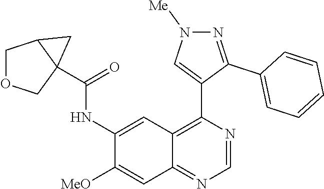 [SMILES: COc1cc2ncnc(-c3cn(C)nc3-c3ccccc3)c2cc1NC(=O)C12COCC1C2]

To a solution of 7-methoxy-4-(1-methyl-3-phenyl-1H-pyrazol-4-yl)quinazolin-6-amine (Preparation 190, 19.4 mg, 0.059 mmol) and 3-oxabicyclo[3.1.0]hexane-1-carboxylic acid (15 mg, 0.117 mmol) in EtOAc (1 mL) was added DIPEA (45.4 mg, 0.351 mmol) and HATU (89 mg, 0.234 mmol) and the mixture stirred at 70° C. for 3 h. DMAP (14.3 mg, 0.117 mmol) was added and the mixture stirred at 70° C. for 5 h. The reaction mixture was filtered, the filtrate was concentrated under reduced pressure and the residue purified by prep-HPLC (Phenomenex Gemini-NX C18 30×75 mm, 3 μm; 20-45% MeCN/H₂O (MeCN/H₂O (10 mM NH₄HCO₃+0.05% NH₄OH)) to afford the title compound as a yellow solid (1.7 mg, 3.3%). LCMS: m/z=442 [M+H]⁺; ¹H NMR (400 MHZ, CDCl₃) δ: 9.10 (s, 1H), 8.90 (s, 1H), 7.87 (s, 1H), 7.86 (s, 1H), 7.40 (s, 1H), 7.39-7.37 (m, 2H), 7.19-7.18 (m, 3H), 4.10 (s, 3H), 4.08 (s, 3H), 3.91-3.83 (m, 2H), 2.18-2.14 (m, 1H), 1.58-1.46 (m, 2H), 1.09 (t, 1H), 0.88-0.83 (m, 1H).

### Example 279: N-(7-(difluoromethoxy)-4-(1-methyl-3-phenyl-1H-pyrazol-4-yl)quinazolin-6-yl)cyclopropanecarboxamide

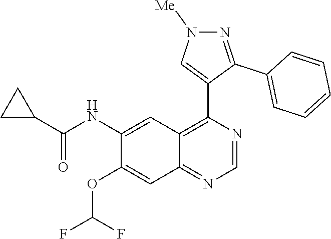 [SMILES: Cn1cc(-c2ncnc3cc(OC(F)F)c(NC(=O)C4CC4)cc23)c(-c2ccccc2)n1]

Part 1. To a mixture of 7-methoxy-4-(1-methyl-3-phenyl-1H-pyrazol-4-yl)quinazolin-6-amine (Preparation 190, 200 mg, 0.604 mmol) and cyclopropanecarboxylic acid (156 mg, 1.81 mmol) in pyridine (2.5 mL) was added EDCl (174 mg, 0.905 mmol) and the mixture stirred at 60° C. for 14 h. The reaction mixture was concentrated under reduced pressure and residue purified by prep-TLC (10:1 EtOAc/EtOH) to give N-(7-methoxy-4-(1-methyl-3-phenyl-1H-pyrazol-4-yl)quinazolin-6-yl)cyclopropanecarboxamide as a yellow solid (300 mg, crude) which was used without further purification.

Part 2. To a mixture of N-(7-methoxy-4-(1-methyl-3-phenyl-1H-pyrazol-4-yl)quinazolin-6-yl)cyclopropanecarboxamide (Part 1, 200 mg, 0.500 mmol) in DMF (2 mL) was added NaSMe (330 mg, 4.71 mmol) and the mixture stirred at 120° C. for 1 h under N₂. The reaction mixture was adjusted to pH 5 with 1 M HCl and partitioned between EtOAc (5 mL) and H₂O (3 mL). The aqueous phase was extracted with EtOAc (3×5 mL) and the combined organics dried (Na₂SO₄) and evaporated to dryness in vacuo to give N-(7-hydroxy-4-(1-methyl-3-phenyl-1H-pyrazol-4-yl)quinazolin-6-yl)cyclopropanecarboxamide as a yellow oil (200 mg, crude) which was used without further purification.

Part 3. To a mixture of N-(7-hydroxy-4-(1-methyl-3-phenyl-1H-pyrazol-4-yl)quinazolin-6-yl)cyclopropanecarboxamide (Part 2, 100 mg, 0.259 mmol) and (2-chloro-2,2-difluoro-acetyl)oxysodium (59.3 mg, 0.389 mmol) in DMF (2 mL) was added K₂CO₃ (53.8 mg, 0.389 mmol) and the mixture stirred at 80° C. for 2 h. The reaction mixture was concentrated under reduced pressure and the residue purified by prep-HPLC (Phenomenex Gemini-NX C18 30×75 mm, 3 μm; 30-60% MeCN/H₂O (10 mM NH₄HCO₃+0.05% NH₄OH)) to afford the title compound as a pale yellow solid (28 mg, 24%). LCMS: m/z=436 [M+H]⁺; ¹H NMR (400 MHZ, CDCl₃) δ: 9.08 (s, 1H), 8.94 (s, 1H), 7.83 (s, 1H), 7.79 (s, 1H), 7.58 (s, 1H), 7.28 (dd, 2H), 7.14-7.08 (m, 3H), 6.93-6.57 (m, 1H), 4.00 (s, 3H), 1.51-1.47 (m, 1H), 1.05-1.01 (m, 2H), 0.87-0.84 (m, 2H).

### Example 280 and 281: (R)—N-(7-methoxy-4-(1-methyl-3-phenyl-1H-pyrazol-4-yl)quinazolin-6-yl)-2-(tetrahydrofuran-2-yl) acetamide and (S)—N-(7-methoxy-4-(1-methyl-3-phenyl-1H-pyrazol-4-yl)quinazolin-6-yl)-2-(tetrahydrofuran-2-yl) acetamide

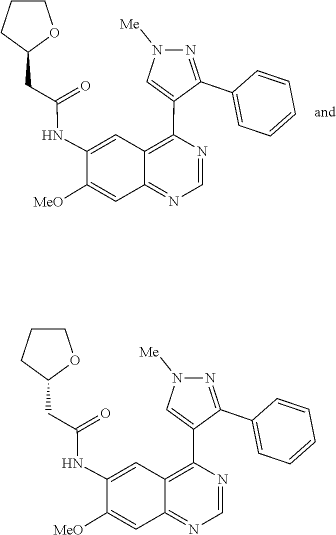 [SMILES: COc1cc2ncnc(-c3cn(C)nc3-c3ccccc3)c2cc1NC(=O)C[C@H]1CCCO1 ; COc1cc2ncnc(-c3cn(C)nc3-c3ccccc3)c2cc1NC(=O)C[C@@H]1CCCO1]

Part 1. Pyridine (1 mL) and T.P.R (1 mL) were added to a mixture of 7-methoxy-4-(1-methyl-3-phenyl-1H-pyrazol-4-yl)quinazolin-6-amine (Preparation 190, 66.3 mg, 0.20 mmol) and 2-(tetrahydrofuran-2-yl) acetic acid (26 mg, 0.20 mmol) in THF (1 mL) and the mixture shaken at 50° C. for 5 h. The reaction mixture was evaporated to dryness by Speedvac. The residue was purified by prep-HPLC to give N-(7-methoxy-4-(1-methyl-3-phenyl-1H-pyrazol-4-yl)quinazolin-6-yl)-2-(tetrahydrofuran-2-yl) acetamide.

Part 2. N-(7-methoxy-4-(1-methyl-3-phenyl-1H-pyrazol-4-yl)quinazolin-6-yl)-2-(tetrahydrofuran-2-yl) acetamide (Part 1, 41 mg, 92.4 μmol) in MeOH was purified by chiral-HPLC (CHIRALPAK AD-H, 20×250 mm, 5 μm; 40% MeOH/Hex (0.5% 2M NH₃—MeOH) to afford:

Peak 1: yellow solid 6.6 mg. Example 280: (R)—N-(7-methoxy-4-(1-methyl-3-phenyl-1H-pyrazol-4-yl)quinazolin-6-yl)-2-(tetrahydrofuran-2-yl) acetamide or (S)—N-(7-methoxy-4-(1-methyl-3-phenyl-1H-pyrazol-4-yl)quinazolin-6-yl)-2-(tetrahydrofuran-2-yl) acetamide. LCMS: m/z=444 [M+H]⁺; ¹H NMR (400 MHZ, DMSO-d₆) δ: 9.49 (s, 1H), 9.04 (s, 1H), 8.77 (s, 1H), 8.18 (s, 1H), 7.44 (s, 1H), 7.27 (ddt, 2H), 7.19 (tt, 3H), 4.12 (qd, 1H), 4.07 (s, 3H), 4.03 (s, 3H), 3.81 (ddd, 1H), 3.65 (td, 1H), 2.66 (dd, 1H), 2.56 (dd, 1H), 1.98 (dddd, 1H), 1.93-1.75 (m, 2H), 1.48 (ddt, 1H), 1.24 (s, 1H).

Peak 2: yellow solid 8.4 mg. Example 281: (S)—N-(7-methoxy-4-(1-methyl-3-phenyl-1H-pyrazol-4-yl)quinazolin-6-yl)-2-(tetrahydrofuran-2-yl) acetamide or (R)—N-(7-methoxy-4-(1-methyl-3-phenyl-1H-pyrazol-4-yl)quinazolin-6-yl)-2-(tetrahydrofuran-2-yl) acetamide. LCMS: m/z=514.35 [M+H]⁺, ¹H NMR (400 MHz, DMSO-d₆) δ: 9.49 (s, 1H), 9.04 (s, 1H), 8.77 (d, 1H), 8.18 (s, 1H), 7.44 (s, 1H), 7.27 (dtd, 2H), 7.19 (tt, 3H), 4.17-4.08 (m, 7H), 3.81 (ddd, 1H), 3.65 (td, 1H), 2.66 (dd, 2H), 1.98 (dddd, 1H), 1.93-1.75 (m, 2H), 1.48 (ddt, 1H), 1.28-1.17 (m, 1H).

### Example 282 and 283: (1S,2R)—N-(7-methoxy-4-(1-methyl-3-phenyl-1H-pyrazol-4-yl)quinazolin-6-yl)-2-methylcyclopropane-1-carboxamide and (1R,2S)—N-(7-methoxy-4-(1-methyl-3-phenyl-1H-pyrazol-4-yl)quinazolin-6-yl)-2-methylcyclopropane-1-carboxamide

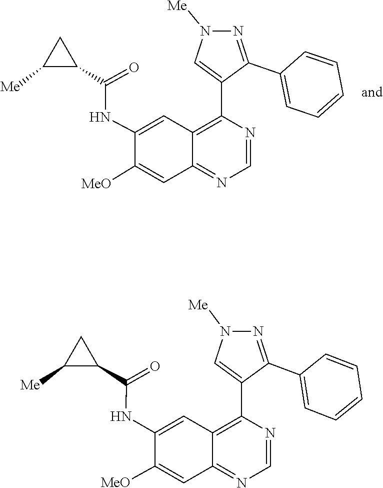 [SMILES: COc1cc2ncnc(-c3cn(C)nc3-c3ccccc3)c2cc1NC(=O)[C@H]1C[C@H]1C ; COc1cc2ncnc(-c3cn(C)nc3-c3ccccc3)c2cc1NC(=O)[C@@H]1C[C@@H]1C]

Part 1. Pyridine (1 mL) and T₃PR (1 mL) were added to a mixture of 7-methoxy-4-(1-methyl-3-phenyl-1H-pyrazol-4-yl)quinazolin-6-amine (Preparation 190, 66.3 mg, 0.20 mmol) and cis-rac-(1R,2S)-2-methylcyclopropane-1-carboxylic acid (20 mg, 0.20 mmol) in THF (1 mL) and the mixture shaken at 50° C. for 5 h. The reaction mixture was evaporated to dryness by Speedvac and the residue purified by prep-HPLC to give cis-rac-(1R,2S)—N-(7-methoxy-4-(1-methyl-3-phenyl-1H-pyrazol-4-yl)quinazolin-6-yl)-2-(trifluoromethyl)cyclopropane-1-carboxamide.

Part 2. cis-Rac-(1R,2S)—N-(7-methoxy-4-(1-methyl-3-phenyl-1H-pyrazol-4-yl)quinazolin-6-yl)-2-methylcyclopropane-1-carboxamide (Part 1, 34 mg, 82.2 μmol) in MeOH was purified by chiral-HPLC (CHIRALPAK IE, 20×250 mm, 5 μm; 10% EtOH/MTBE (0.5% 2M NH₃—MeOH) to afford:

Peak 1, white solid (2 mg). Example 282: (1S,2R)—N-(7-methoxy-4-(1-methyl-3-phenyl-1H-pyrazol-4-yl)quinazolin-6-yl)-2-methylcyclopropane-1-carboxamide or (1R,2S)—N-(7-methoxy-4-(1-methyl-3-phenyl-1H-pyrazol-4-yl)quinazolin-6-yl)-2-methylcyclopropane-1-carboxamide. LCMS: m/z=414 [M+H]⁺; ¹H NMR (400 MHZ, DMSO-d₆) δ: 9.52 (s, 1H), 9.04 (s, 1H), 8.67 (d, 1H), 8.18 (s, 1H), 7.42 (s, 1H), 7.29-7.19 (m, 2H), 7.22-7.12 (m, 3H), 4.04 (d, 6H), 2.12 (td, 1H), 1.28-1.18 (m, 1H), 1.06 (d, 3H), 0.93 (td, 1H), 0.75 (ddd, 1H).

Peak 2, white solid (17.4 mg). Example 283: (1R,2S)—N-(7-methoxy-4-(1-methyl-3-phenyl-1H-pyrazol-4-yl)quinazolin-6-yl)-2-methylcyclopropane-1-carboxamide or (1S,2R)—N-(7-methoxy-4-(1-methyl-3-phenyl-1H-pyrazol-4-yl)quinazolin-6-yl)-2-methylcyclopropane-1-carboxamide. LCMS: m/z=414 [M+H]⁺; ¹H NMR (400 MHZ, DMSO-d₆) δ: 9.62 (s, 1H), 9.02 (s, 1H), 8.70 (s, 1H), 8.16 (s, 1H), 7.43 (s, 1H), 7.30-7.21 (m, 2H), 7.24-7.13 (m, 3H), 4.08 (s, 3H), 4.01 (s, 3H), 1.93 (dt, 1H), 1.26-1.14 (m, 1H), 1.08 (d, 3H), 0.98 (ddd, 1H), 0.63 (ddd, 1H).

### Example 284 and 285: (3S,4S)-3-fluoro-N-(7-methoxy-4-(1-methyl-3-phenyl-1H-pyrazol-4-yl)quinazolin-6-yl)-1-methylpiperidine-4-carboxamide and (3R,4R)-3-fluoro-N-(7-methoxy-4-(1-methyl-3-phenyl-1H-pyrazol-4-yl)quinazolin-6-yl)-1-methylpiperidine-4-carboxamide

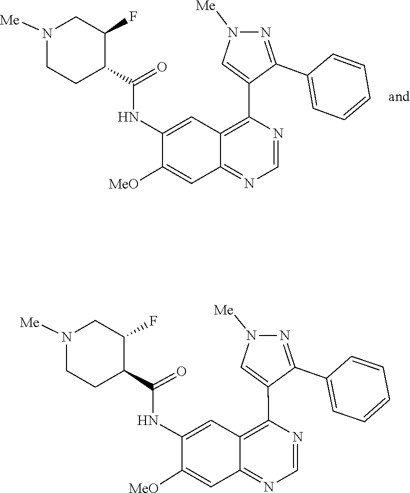 [SMILES: COc1cc2ncnc(-c3cn(C)nc3-c3ccccc3)c2cc1NC(=O)[C@@H]1CCN(C)C[C@H]1F ; COc1cc2ncnc(-c3cn(C)nc3-c3ccccc3)c2cc1NC(=O)[C@H]1CCN(C)C[C@@H]1F]

Part 1. Pyridine (1 mL) and T₃P® (1 mL) were added to a mixture of 7-methoxy-4-(1-methyl-3-phenyl-1H-pyrazol-4-yl)quinazolin-6-amine (Preparation 190, 66.3 mg, 0.20 mmol) and trans-rac-(3R,4R)-3-fluoro-1-methylpiperidine-4-carboxylic acid (32.2 mg, 0.20 mmol) in THF (1 mL) and the mixture shaken at 50° C. for 5 h. The reaction mixture was evaporated to dryness by Speedvac and the residue purified by prep-HPLC (Xtimate C18, 25×150 mm, 5 mm; MeCN/H₂O (0.05% v/v NH+OH/H₂O)) to give trans-rac-(3R,4R)-3-fluoro-N-(7-methoxy-4-(1-methyl-3-phenyl-1H-pyrazol-4-yl)quinazolin-6-yl)-1-methylpiperidine-4-carboxamide.

Part 2. trans-rac-(3R,4R)-3-fluoro-N-(7-methoxy-4-(1-methyl-3-phenyl-1H-pyrazol-4-yl)quinazolin-6-yl)-1-methylpiperidine-4-carboxamide (Part 1, 29.5 mg, 62.1 μmol) in MeOH was purified by chiral-HPLC (CHIRALPAK 1H, 20×250 mm, 5 μm; 10% EtOH/MTBE (0.5% 2M NH₃—MeOH) to afford:

Peak 1, off-white solid (3.8 mg). Example 284: (3S,4S)-3-fluoro-N-(7-methoxy-4-(1-methyl-3-phenyl-1H-pyrazol-4-yl)quinazolin-6-yl)-1-methylpiperidine-4-carboxamide or (3R,4R)-3-fluoro-N-(7-methoxy-4-(1-methyl-3-phenyl-1H-pyrazol-4-yl)quinazolin-6-yl)-1-methylpiperidine-4-carboxamide. LCMS: m/z=475 [M+H]⁺; ¹H NMR (400 MHz, DMSO-d₆) δ: 9.76 (s, 1H), 9.07 (s, 1H), 8.70 (s, 1H), 8.21 (s, 1H), 7.46 (s, 1H), 7.24 (dd, 5H), 4.96 (s, 1H), 4.05 (d, 6H), 3.12 (s, 4H), 2.09 (s, 2H), 2.01 (q, 1H), 1.69 (s, 1H).

Peak 2, off-white solid (2 mg). Example 285: (3R,4R)-3-fluoro-N-(7-methoxy-4-(1-methyl-3-phenyl-1H-pyrazol-4-yl)quinazolin-6-yl)-1-methylpiperidine-4-carboxamide or (3S,4S)-3-fluoro-N-(7-methoxy-4-(1-methyl-3-phenyl-1H-pyrazol-4-yl)quinazolin-6-yl)-1-methylpiperidine-4-carboxamide. LCMS: m/z=475 [M+H]⁺; ¹H NMR (400 MHZ, DMSO-d₆) δ: 9.58 (s, 1H), 9.05 (s, 1H), 8.74 (d, 1H), 8.21 (s, 1H), 7.43 (s, 1H), 7.20-7.14 (m, 5H), 4.80 (s, 1H), 4.05 (d, 5H), 3.17 (s, 1H), 2.86 (s, 3H), 2.53 (d, 3H), 1.85 (s, 2H), 1.24 (s, 1H).

### Example 286 and 287: (1R,2R)—N-(7-methoxy-4-(1-methyl-3-phenyl-1H-pyrazol-4-yl)quinazolin-6-yl)-2-(trifluoromethyl)cyclopropane-1-carboxamide and (1S,2S)—N-(7-methoxy-4-(1-methyl-3-phenyl-1H-pyrazol-4-yl)quinazolin-6-yl)-2-(trifluoromethyl)cyclopropane-1-carboxamide

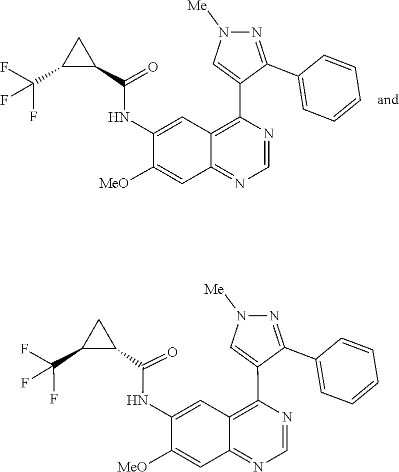 [SMILES: COc1cc2ncnc(-c3cn(C)nc3-c3ccccc3)c2cc1NC(=O)[C@@H]1C[C@H]1C(F)(F)F ; COc1cc2ncnc(-c3cn(C)nc3-c3ccccc3)c2cc1NC(=O)[C@H]1C[C@@H]1C(F)(F)F]

Part 1. To a solution of 7-methoxy-4-(1-methyl-3-phenyl-1H-pyrazol-4-yl)quinazolin-6-amine (Preparation 190, 100 mg, 0.302 mmol) in pyridine (3 mL) was added EDCl (93 mg, 0.483 mmol) and trans-rac-(1R,2S)-2-(trifluoromethyl)cyclopropanecarboxylic acid (69.7 mg, 0.453 mmol) and the mixture stirred at 70° C. for 16 h. The reaction mixture was evaporated to dryness and the residue diluted with H₂O and extracted with DCM (2×). The combined extracts were dried (Na2SO4) and evaporated to dryness. The residue was purified by column chromatography (ISCO, 0-100% EtOAc/DCM followed by 25% MeOH/DCM) to afford trans-rac-(1R,2R)—N-(7-methoxy-4-(1-methyl-3-phenyl-1H-pyrazol-4-yl)quinazolin-6-yl)-2-(trifluoromethyl)cyclopropane-1-carboxamide (105 mg. 74%)

Part 2. trans-rac-(1R,2R)—N-(7-methoxy-4-(1-methyl-3-phenyl-1H-pyrazol-4-yl)quinazolin-6-yl)-2-(trifluoromethyl)cyclopropane-1-carboxamide (Part 1) in MeOH was purified by chiral-HPLC (LUX Cellulose-2, 21.2×250 mm, 5 μm; 20% IPA/MTBE (0.5% 2M NH₃—MeOH) to afford:

Peak 1, white solid (24.3 mg). Example 286: (1R,2R)—N-(7-methoxy-4-(1-methyl-3-phenyl-1H-pyrazol-4-yl)quinazolin-6-yl)-2-(trifluoromethyl)cyclopropane-1-carboxamide or (1S,2S)—N-(7-methoxy-4-(1-methyl-3-phenyl-1H-pyrazol-4-yl)quinazolin-6-yl)-2-(trifluoromethyl)cyclopropane-1-carboxamide. LCMS: m/z=468 [M+H]⁺; ¹H NMR (400 MHZ, DMSO-d₆) δ: 10.03 (s, 1H), 9.04 (s, 1H), 8.70 (s, 1H), 8.16 (s, 1H), 7.45 (s, 1H), 7.26-7.23 (m, 2H), 7.19-7.16 (m, 3H), 4.08 (s, 3H), 4.01 (s, 3H), 2.81-2.76 (m, 1H), 2.26-2.19 (m, 1H), 1.25 (t, 2H).

Peak 2, white solid (23.9 mg). Example 287: (1S,2S)—N-(7-methoxy-4-(1-methyl-3-phenyl-1H-pyrazol-4-yl)quinazolin-6-yl)-2-(trifluoromethyl)cyclopropane-1-carboxamide or (1R,2R)—N-(7-methoxy-4-(1-methyl-3-phenyl-1H-pyrazol-4-yl)quinazolin-6-yl)-2-(trifluoromethyl)cyclopropane-1-carboxamide. LCMS: m/z=468 [M+H]⁺; ¹H NMR (400 MHZ, DMSO-d₆) δ: 10.02 (s, 1H), 9.04 (s, 1H), 8.70 (s, 1H), 8.16 (s, 1H), 7.45 (s, 1H), 7.26-7.23 (m, 2H), 7.19-7.16 (m, 3H), 4.08 (s, 3H), 4.01 (s, 3H), 2.81-2.76 (m, 1H), 2.26-2.19 (m, 1H), 1.25 (t, 2H).

### Example 288 and 289: (1R,5R)—N-(4-(1-ethyl-3-phenyl-1H-pyrazol-4-yl)-7-methoxyquinazolin-6-yl)-3-oxabicyclo[3.1.0]hexane-1-carboxamide and (1S,5S)—N-(4-(1-ethyl-3-phenyl-1H-pyrazol-4-yl)-7-methoxyquinazolin-6-yl)-3-oxabicyclo[3.1.0]hexane-1-carboxamide

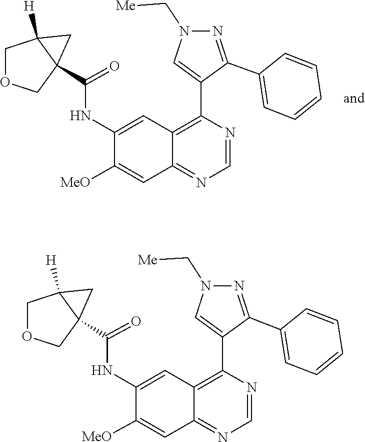 [SMILES: CCn1cc(-c2ncnc3cc(OC)c(NC(=O)[C@@]45COC[C@@H]4C5)cc23)c(-c2ccccc2)n1 ; CCn1cc(-c2ncnc3cc(OC)c(NC(=O)[C@]45COC[C@H]4C5)cc23)c(-c2ccccc2)n1]

Part 1. XPhos (33 mg, 69.3 μmol), Pd₂(dba)₃ (31.6 mg, 34.6 μmol) and K₂CO₃ (71.7 mg. 0.520 mmol) were added to 6-bromo-4-(1-ethyl-3-phenyl-1H-pyrazol-4-yl)-7-methoxyquinazoline (Preparation 94, 120 mg, 0.347 mmol) and 3-oxabicyclo[3.1.0]hexane-1-carboxamide (Preparation 64, 66.6 mg, 0.520 mmol) in dioxane (6 mL) at rt and the reaction mixture heated to 100° C. for 2 h under N₂. The resulting solution was extracted with EtOAc (3×40 mL) and the combined extracts dried (Na₂SO₄) and evaporated to dryness. The residue was purified by prep-HPLC (XBridge Shield RP18 OBD Column, 30×150 mm, 5 μm; 8-54% MeCN/H₂O (10 mM NH₄HCO₃+0.1% NH₄OH)) to afford N-(4-(1-ethyl-3-phenyl-1H-pyrazol-4-yl)-7-methoxyquinazolin-6-yl)-3-oxabicyclo[3.1.0]hexane-1-carboxamide as a white solid (30 mg, 18%). LCMS: m/z=456 [M+H]⁺.

Part 2. N-(4-(1-ethyl-3-phenyl-1H-pyrazol-4-yl)-7-methoxyquinazolin-6-yl)-3-oxabicyclo[3.1.0]hexane-1-carboxamide (Part 1, 30 mg, 65.8 μmol) in MeOH was purified by chiral-HPLC (Chiral Art Cellulose-SB, 20×250 mm, 5 μm; 10% IPA/MTBE (0.5% 2M NH₃—MeOH) to afford:

Peak 1, white solid (11.5 mg). Example 288: (1R,5R)—N-(4-(1-ethyl-3-phenyl-1H-pyrazol-4-yl)-7-methoxyquinazolin-6-yl)-3-oxabicyclo[3.1.0]hexane-1-carboxamide or (1S,5S)—N-(4-(1-ethyl-3-phenyl-1H-pyrazol-4-yl)-7-methoxyquinazolin-6-yl)-3-oxabicyclo[3.1.0]hexane-1-carboxamide. LCMS: m/z=456 [M+H]⁺; ¹H NMR (400 MHZ, DMSO-d₆) δ: 9.05 (s, 1H), 8.68 (s, 1H), 8.46 (s, 1H), 8.22 (s, 1H), 7.46 (s, 1H), 7.25 (ddt, 5H), 4.31 (q, 2H), 4.05 (s, 3H), 3.95 (s, 2H), 3.77-3.67 (m, 2H), 2.16 (ddd, 1H), 1.52 (t, 3H), 1.38 (dd, 1H), 0.87 (t, 1H).

Peak 2, white solid (12.3 mg). Example 289: (1S,5S)—N-(4-(1-ethyl-3-phenyl-1H-pyrazol-4-yl)-7-methoxyquinazolin-6-yl)-3-oxabicyclo[3.1.0]hexane-1-carboxamide or (1R,5R)—N-(4-(1-ethyl-3-phenyl-1H-pyrazol-4-yl)-7-methoxyquinazolin-6-yl)-3-oxabicyclo[3.1.0]hexane-1-carboxamide. LCMS: m/z=456 [M+H]⁺; ¹H NMR (400 MHZ, DMSO-d₆) δ: 9.05 (s, 1H), 8.68 (s, 1H), 8.46 (s, 1H), 8.22 (s, 1H), 7.46 (s, 1H), 7.25 (dq, 5H), 4.31 (q, 2H), 4.05 (s, 3H), 3.95 (s, 2H), 3.79-3.67 (m, 2H), 2.16 (ddd, 1H), 1.52 (t, 3H), 1.38 (dd, 1H), 0.87 (t, 1H).

### Example 290 and 291: (1R,5R)—N-(7-ethoxy-4-(1-methyl-3-phenyl-1H-pyrazol-4-yl)quinazolin-6-yl)-3-oxabicyclo[3.1.0]hexane-1-carboxamide and (1S,5S)—N-(7-ethoxy-4-(1-methyl-3-phenyl-1H-pyrazol-4-yl)quinazolin-6-yl)-3-oxabicyclo[3.1.0]hexane-1-carboxamide

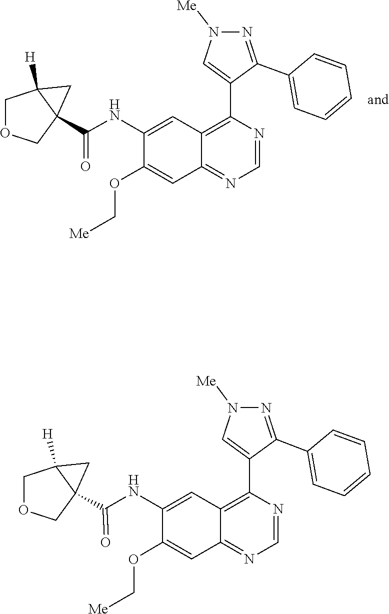 [SMILES: CCOc1cc2ncnc(-c3cn(C)nc3-c3ccccc3)c2cc1NC(=O)[C@@]12COC[C@@H]1C2 ; CCOc1cc2ncnc(-c3cn(C)nc3-c3ccccc3)c2cc1NC(=O)[C@]12COC[C@H]1C2]

The title compounds were prepared from 3-oxabicyclo[3.1.0]hexane-1-carboxamide (Preparation 64) and 6-bromo-7-ethoxy-4-(1-methyl-3-phenyl-1H-pyrazol-4-yl)quinazoline (Preparation 90) using an analogous 2-part method as described for Example 288 and 289. Chiral-HPLC (Chiral Art Cellulose-SB, 20×250 mm, 5 μm; 5% EtOH/MTBE (0.5% 2M NH₃—MeOH) to afford:

Peak 1, white solid (15.3 mg). Example 290: (1R,5R)—N-(7-ethoxy-4-(1-methyl-3-phenyl-1H-pyrazol-4-yl)quinazolin-6-yl)-3-oxabicyclo[3.1.0]hexane-1-carboxamide or (1S,5S)—N-(7-ethoxy-4-(1-methyl-3-phenyl-1H-pyrazol-4-yl)quinazolin-6-yl)-3-oxabicyclo[3.1.0]hexane-1-carboxamide. LCMS: m/z=456 [M+H]⁺; ¹H NMR (400 MHZ, DMSO-d₆) δ: 9.05 (s, 1H), 8.59 (s, 1H), 8.45 (s, 1H), 8.17 (s, 1H), 7.44 (s, 1H), 7.23-7.16 (m, 5H), 4.32 (q, 2H), 4.02 (s, 5H), 3.78-3.67 (m, 2H), 2.15 (ddd, 1H), 1.46 (t, 3H), 1.38 (dd, 1H), 1.23 (s, 1H), 0.89 (t, 1H).

Peak 2, white solid (16.4 mg). Example 291: (1S,5S)—N-(7-ethoxy-4-(1-methyl-3-phenyl-1H-pyrazol-4-yl)quinazolin-6-yl)-3-oxabicyclo[3.1.0]hexane-1-carboxamide or (1R,5R)—N-(7-ethoxy-4-(1-methyl-3-phenyl-1H-pyrazol-4-yl)quinazolin-6-yl)-3-oxabicyclo[3.1.0]hexane-1-carboxamide. LCMS: m/z=456 [M+H]⁺; ¹H NMR (400 MHZ, DMSO-d₆) δ: 9.05 (s, 1H), 8.59 (s, 1H), 8.45 (s, 1H), 8.17 (s, 1H), 7.44 (s, 1H), 7.30-7.23 (m, 2H), 7.23-7.16 (m, 3H), 4.32 (q, 2H), 4.02 (s, 5H), 3.78-3.67 (m, 2H), 2.15 (ddd, 1H), 1.46 (t, 3H), 1.38 (dd, 1H), 1.23 (s, 1H), 0.89 (t, 1H).

### Example 292 and 293: (1S,5S)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide or (1R,5R)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide

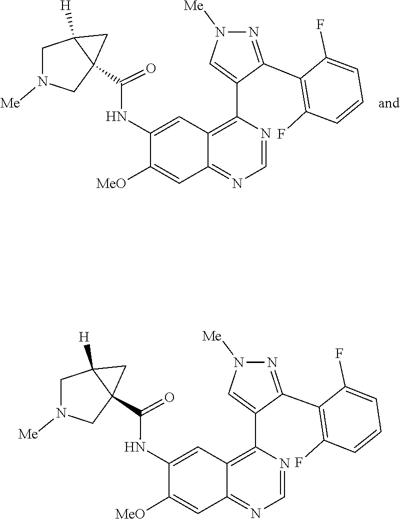 [SMILES: COc1cc2ncnc(-c3cn(C)nc3-c3c(F)cccc3F)c2cc1NC(=O)[C@@]12C[C@@H]1CN(C)C2 ; COc1cc2ncnc(-c3cn(C)nc3-c3c(F)cccc3F)c2cc1NC(=O)[C@]12C[C@H]1CN(C)C2]

Part 1. A mixture of Cs₂CO₃ (78.8 mg, 242 μmol), Pd₂(dba)₃ (14.8 mg, 16.2 μmol), Xphos (9.36 mg, 16.2 μmol), 6-bromo-4-(3-(2,6-difluorophenyl)-1-methyl-1H-pyrazol-4-yl)-7-methoxyquinazoline (Preparation 92, 70 mg, 162 μmol) and tert-butyl 1-carbamoyl-3-azabicyclo[3.1.0]hexane-3-carboxylate (Preparation 65, 73.3 mg, 324 μmol) in 1,4-dioxane (6 mL) was stirred at 100° C. for 5 h. The mixture was diluted with EtOAc (100 mL) and washed with water (2×50 mL). The combined organics were dried (Na₂SO₄) and evaporated to dryness. The residue was purified by column chromatography (18:1 DCM/MeOH) to afford tert-butyl 1-((4-(3-(2,6-difluorophenyl)-1-methyl-1H-pyrazol-4-yl)-7-methoxyquinazolin-6-yl)carbamoyl)-3-azabicyclo[3.1.0]hexane-3-carboxylate as a yellow solid (45 mg, 64%). LCMS: m/z=475 [M+H]⁺.

Part 2. TFA (1 mL) was added to tert-butyl 1-((4-(3-(2,6-difluorophenyl)-1-methyl-1H-pyrazol-4-yl)-7-methoxyquinazolin-6-yl)carbamoyl)-3-azabicyclo[3.1.0]hexane-3-carboxylate (Part 1, 45 mg, 78 μmol) in DCM (3 mL) at rt and the resulting mixture stirred for 1 h. The reaction mixture was evaporated to dryness in vacuo to afford N-(4-(3-(2,6-difluorophenyl)-1-methyl-1H-pyrazol-4-yl)-7-methoxyquinazolin-6-yl)-3-azabicyclo[3.1.0]hexane-1-carboxamide as a white solid (35 mg. 56%). LCMS: m/z=456 [M+H]⁺.

Part 3. NaBH(OAc)₃ (30.9 mg, 146 μmol) was added to N-(4-(3-(2,6-difluorophenyl)-1-methyl-1H-pyrazol-4-yl)-7-methoxyquinazolin-6-yl)-3-azabicyclo[3.1.0]hexane-1-carboxamide (Part 2, 35 mg, 73.4 μmol) and HCHO (0.1 mL, 37% in water) in DCM (5 mL) at rt and the mixture stirred at rt for 2 h. The mixture was diluted with EtOAc (100 mL) and washed with brine (2×50 mL) and the combined organics dried (Na₂SO₄) and evaporated to dryness in vacuo. The residue was purified by prep-HPLC (CHIRALPAK ID, 30×250 mm, 5 μm; 30% MeOH/MTBE (0.5% 2M NH₃—MeOH)) to give N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide as an off-white solid (20 mg).

Part 4. N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide (Part 3, 12 mg, 24.4 μmol) in MeOH was purified by chiral-HPLC (Chiral ART Amylose-SA, 20×250 mm, 5 μm; 5% (50% MeOH/DCM)/MTBE (0.5% 2M NH₃—MeOH) to afford:

Peak 1, off-white solid. Example 292: (1S,5S)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide or (1R,5R)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide. LCMS: m/z=491 [M+H]⁺; ¹H NMR (400 MHZ, DMSO-d₆) δ: 8.84 (s, 1H), 8.72 (s, 1H), 8.58 (s, 1H), 8.41 (s, 1H), 7.45-7.33 (m, 2H), 7.01 (t, 2H), 4.06 (d, 6H), 2.92 (d, 2H), 2.62 (d, 1H), 2.37 (d, 1H), 2.29 (s, 3H), 1.34 (t, 1H), 1.24 (s, 1H), 1.21 (d, 1H).

Peak 2, off-white solid. Example 293: (1R,5R)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide or (1S,5S)—N-(4-(3-(2,6-difluorophenyl)-1-methyl-1H-pyrazol-4-yl)-7-methoxyquinazolin-6-yl)-3-methyl-3-azabicyclo[3.1.0]hexane-1-carboxamide. LCMS: m/z=491 [M+H]⁺; ¹H NMR (400 MHZ, DMSO-d₆) δ: 8.84 (s, 1H), 8.72 (s, 1H), 8.58 (s, 1H), 8.40 (s, 1H), 7.45-7.33 (m, 2H), 7.01 (t, 2H), 4.06 (d, 6H), 2.62 (d, 2H), 2.36 (dd, 1H), 2.29 (s, 3H), 1.99 (s, 1H), 1.34 (t, 1H), 1.24 (s, 1H), 1.21 (s, 1H).

### Example 294: N-(7-methoxy-4-(1-methyl-3-phenyl-1H-pyrazol-4-yl)quinazolin-6-yl)-1-methyl-1H-pyrazole-4-carboxamide

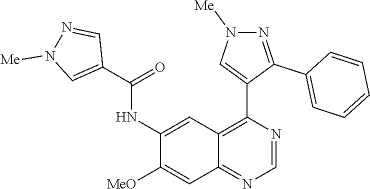 [SMILES: COc1cc2ncnc(-c3cn(C)nc3-c3ccccc3)c2cc1NC(=O)c1cnn(C)c1]

Part 1:1-Methyl-1H-pyrazole-4-carboxylic acid (200 mg, 1.58 mmol) in SOCl₂ (3 mL) was stirred at 60° C. for 1 h. The reaction mixture was evaporated to dryness in vacuo to afford 1-methyl-1H-pyrazole-4-carbonyl chloride which was used in Part 2 without further purification.

Part 2: Pyridine (7.14 mg, 90.5 μmol) was added dropwise to a mixture of 7-methoxy-4-(1-methyl-3-phenyl-1H-pyrazol-4-yl)quinazolin-6-amine (P 190, 60 mg, 0.181 mmol) and 1-methyl-1H-pyrazole-4-carbonyl chloride (Part 1, 39.1 mg, 0.271 mmol) in DCM and the mixture stirred at rt for 1 h. The reaction was evaporated to dryness in vacuo and the residue was purified by prep-HPLC (YMC-Actus Triart C18 ExRS, 30×150 mm, 5 μm; 11-45% MeCN/H₂O (10 mM NH₄HCO₃+0.1% NH₃·H₂O)) to afford the title compound as a white solid (41.6 mg, 52%). LCMS: m/z=440 [M+H]⁺; ¹H NMR (400 MHZ, DMSO-d₆) δ: 9.19 (s, 1H), 9.06 (s, 1H), 8.58 (s, 1H), 8.38 (s, 1H), 8.21 (s, 1H), 8.01 (d, 1H), 7.49 (s, 1H), 7.35-7.26 (m, 2H), 7.20 (dp, 3H), 4.09 (s, 3H), 4.03 (s, 3H), 3.89 (s, 3H).

### Example 295: N-(5-fluoro-7-methoxy-4-(1-methyl-3-phenyl-1H-pyrazol-4-yl)quinazolin-6-yl)cyclobutanecarboxamide

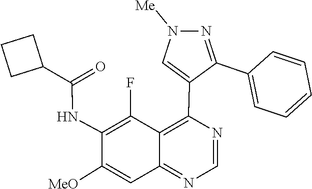 [SMILES: COc1cc2ncnc(-c3cn(C)nc3-c3ccccc3)c2c(F)c1NC(=O)C1CCC1]

Cyclobutanecarbonyl chloride (27 mg, 0.228 mmol) was added to 5-fluoro-7-methoxy-4-(1-methyl-3-phenyl-1H-pyrazol-4-yl)quinazolin-6-amine (Preparation 256, 20 mg, 57.2 μmol) and TEA (29.1 mg, 286 μmol) in DCM (3 mL) at 0° C. and the resulting mixture was stirred at rt for 2 h. The reaction mixture was evaporated to dryness in vacuo and the residue purified by prep-HPLC (XBridge Shield RP18 OBD Column, 30×150 mm, 5 μm; 27-37% MeCN/H₂O (10 mM NH+HCO₃+0.1% NH₄OH)) to afford the title compound as a white solid (11.3 mg, 37%). LCMS: m/z=432 [M+H]⁺; ¹H NMR (300 MHz, DMSO-d₆) δ: 9.18 (s, 1H), 9.03 (s, 1H), 7.91 (d, 1H), 7.26 (d, 1H), 7.14 (dd, 2H), 7.13-7.03 (m, 3H), 3.87 (d, 6H), 3.12 (s, 1H), 2.03 (d, 4H), 1.98 (s, 2H).

### Example 296-319

The title compounds were prepared from the appropriate halide and the appropriate amide using an analogous method to that described for Example 232.

Halide-1: 6-bromo-7-ethoxy-4-(1-methyl-3-phenyl-1H-pyrazol-4-yl)quinazoline (Preparation 90): Halide-2: 6-chloro-7-methoxy-4-(1-methyl-3-phenyl-1H-pyrazol-4-yl)pyrido[3,2-d]pyrimidine (Preparation 242): Halide-3: 2-((6-bromo-4-(1-methyl-3-phenyl-1H-pyrazol-4-yl)quinazolin-7-yl)oxy)-N,N-dimethylethan-1-amine (Preparation 105): Halide-4: 6-bromo-7-methoxy-4-(3-phenyl-1-propyl-1H-pyrazol-4-yl)quinazoline (Preparation 108); Halide-5: 6-bromo-7-(difluoromethoxy)-4-(1-ethyl-3-phenyl-1H-pyrazol-4-yl)quinazoline (Preparation 100): Halide-6: 6-bromo-4-(3-(4-fluorophenyl)-1-methyl-1H-pyrazol-4-yl)-7-methoxyquinazoline (Preparation 97): Halide-7: 6-chloro-7-ethoxy-4-(1-methyl-3-phenyl-1H-pyrazol-4-yl)pyrido[3,2-d]pyrimidine (Preparation 231): Halide-8: 6-bromo-4-(1-ethyl-3-phenyl-1H-pyrazol-4-yl)-7-methoxyquinazoline (Preparation 94): Halide-9: 2-(4-(6-bromo-7-methoxyquinazolin-4-yl)-3-phenyl-1H-pyrazol-1-yl)-N,N-dimethylethan-1-amine (Preparation 297): Halide-10: 6-chloro-4-(1-ethyl-3-phenyl-1H-pyrazol-4-yl)-7-methoxypyrido[3,2-d]pyrimidine (Preparation 239)

| Example | |
|---|---|
| Number | Name/Structure/Halide/RCONH₂/Data |
| 296^{A} | N-(7-ethoxy-4-(1-methyl-3-phenyl-1H-pyrazol-4-yl)quinazolin-6-yl)nicotinamide |
| | 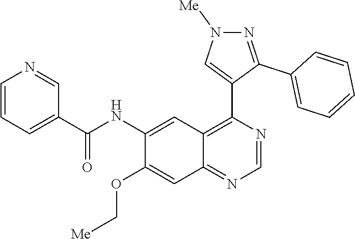 [SMILES: CCOc1cc2ncnc(-c3cn(C)nc3-c3ccccc3)c2cc1NC(=O)c1cccnc1] |
| | Halide-1; RCONH₂: pyridine-2-carboxamide |
| | HPLC: XBridge Shield RP18 OBD Column, 30 x 150 mm, 5μm; 26-36% |
| | MeCN/H₂O (10 mM NH₄HCO₃ + 0.1% NH₄OH) |
| | White solid (61.9 mg, 56%). LCMS: m/z = 451 [M + H]⁺; ¹H NMR (300 MHz, |
| | DMSO-d₆) δ: 9.89 (s, 1H), 9.12-9.03 (m, 2H), 8.78 (dd, 1H), 8.57 (d, 1H), 8.30- |
| | 8.20 (m, 2H), 7.59 (dd, 1H), 7.50 (s, 1H), 7.35-7.26 (m, 2H), 7.25-7.16 (m, 3H), |
| | 4.36 (q, 2H), 4.04 (s, 3H). |
| 297^{A} | 6-cyclopropyl-N-(7-ethoxy-4-(1-methyl-3-phenyl-1H-pyrazol-4-yl)quinazolin-6- |
| | yl)nicotinamide |
| | 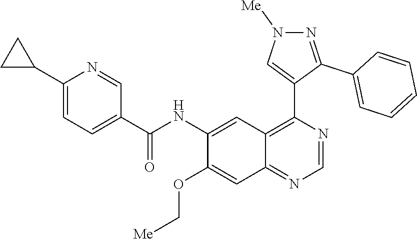 [SMILES: CCOc1cc2ncnc(-c3cn(C)nc3-c3ccccc3)c2cc1NC(=O)c1ccc(C2CC2)nc1] |
| | Halide-1; RCONH₂: 6-cyclopropylnicotinamide |
| | HPLC: XBridge Shield RP18 OBD, 30 x 150 mm, 5 μm; 41-51% MeCN/H₂O (10 |
| | mM NH₄HCO₃ + 0.1% NH₄OH) |
| | White solid (23.4 mg, 20%). LCMS: m/z = 491 [M + H]⁺; ¹H NMR (400 MHz, |
| | DMSO-d₆) δ: 9.69 (s, 1H), 9.08 (s, 1H), 8.90 (d, 1H), 8.59 (d, 1H), 8.21 (s, 1H), |
| | 8.10 (dd, 1H), 7.51-7.43 (m, 2H), 7.31 (dd, 2H), 7.23-7.17 (m, 3H), 4.36 (q, 2H), |
| | 4.04 (s, 3H), 2.22 (m, 1H), 1.48 (t, 3H), 1.10-0.97 (m, 4H). |
| 298^{A} | N-(7-ethoxy-4-(1-methyl-3-phenyl-1H-pyrazol-4-yl)quinazolin-6-yl)-1-ethyl-1H- |
| | pyrazole-4-carboxamide |
| | 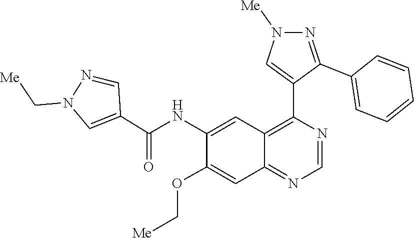 [SMILES: CCOc1cc2ncnc(-c3cn(C)nc3-c3ccccc3)c2cc1NC(=O)c1cnn(CC)c1] |
| | Halide-1; RCONH₂: 1-ethyl-1H-pyrazole-4-carboxamide |
| | HPLC: XBridge Shield RP18 OBD, 30 x 150 mm, 5 μm; 30-45% MeCN/H₂O (10 |
| | mM NH₄HCO₃ + 0.1% NH₄OH) |
| | White solid (75.5 mg, 66%). LCMS: m/z = 469 [M + H]⁺; ¹H NMR (400 MHz, |
| | DMSO-d₆) δ: 9.08 (m, 2H), 8.57 (s, 1H), 8.39 (s, 1H), 8.21 (s, 1H), 8.00 (s, 1H), |
| | 7.47 (s, 1H), 7.30 (m, 2H), 7.20 (m, 3H), 4.37 (q, 2H), 4.19 (q, 2H), 4.03 (s, 3H), |
| | 1.45 (m, 6H). |
| 299 | N-(7-(2-(dimethylamino)ethoxy)-4-(1-methyl-3-phenyl-1H-pyrazol-4- |
| | yl)quinazolin-6-yl)bicyclo[1.1.1]pentane-1-carboxamide |
| | 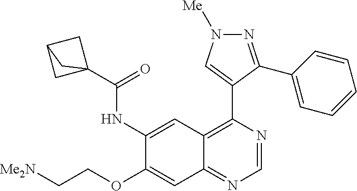 [SMILES: CN(C)CCOc1cc2ncnc(-c3cn(C)nc3-c3ccccc3)c2cc1NC(=O)C12CC(C1)C2] |
| | Halide-3; RCONH₂: bicyclo[1.1.1]pentane-1-carboxamide (Preparation 70). |
| | HPLC: XBridge Shield RP18 OBD, 30 x 150 mm, 5 μm; 34-49% MeCN/H₂O (10 |
| | mM NH₄HCO₃ + 0.1% NH₄OH). |
| | White solid (19.7 mg, 31%). LCMS: m/z = 483 [M + H]⁺; ¹H NMR (400 MHz, |
| | DMSO-d₆) δ: 9.05 (s, 1H), 8.61 (d, 2H), 8.20 (s, 1H), 7.52 (s, 1H), 7.32-7.24 (m, |
| | 2H), 7.24-7.17 (m, 3H), 4.38 (t, 2H), 4.03 (s, 3H), 2.78 (t, 2H), 2.50 (s, 1H), 2.28 |
| | (s, 6H), 2.07 (s, 6H). |
| 300 | N-(7-(2-(dimethylamino)ethoxy)-4-(1-methyl-3-phenyl-1H-pyrazol-4- |
| | yl)quinazolin-6-yl)-2-fluorobenzamide |
| | 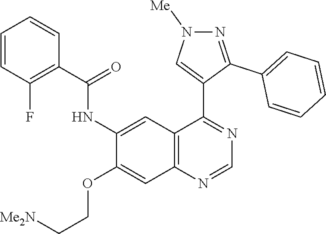 [SMILES: CN(C)CCOc1cc2ncnc(-c3cn(C)nc3-c3ccccc3)c2cc1NC(=O)c1ccccc1F] |
| | Halide-3; RCONH₂: 2-fluorobenzamide. |
| | HPLC: YMC-Actus Triart C18, 30 x 150 mm, 5 μm; 40-70% MeCN/H₂O (10 mM |
| | NH₄HCO₃ + 0.1% NH₄OH) |
| | White solid (15.9 mg, 20%). LCMS: m/z = 511 [M + H]⁺; ¹H NMR (400 MHz, |
| | DMSO-d₆) δ: 9.78 (d, 1H), 9.09 (s, 1H), 8.91 (s, 1H), 8.26 (s, 1H), 7.96-7.87 (m, |
| | 1H), 7.73-7.62 (m, 1H), 7.58 (s, 1H), 7.49-7.37 (m, 2H), 7.30 (tt, 2H), 7.25-7.16 |
| | (m, 3H), 4.41 (t, 2H), 4.06 (s, 3H), 2.76 (t, 2H), 2.23 (s, 6H). |
| 301 | 6-cyclopropyl-N-(7-methoxy-4-(3-phenyl-1-propyl-1H-pyrazol-4-yl)quinazolin-6- |
| | yl)nicotinamide |
| | 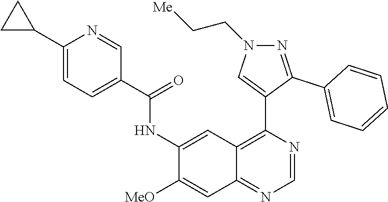 [SMILES: CCCn1cc(-c2ncnc3cc(OC)c(NC(=O)c4ccc(C5CC5)nc4)cc23)c(-c2ccccc2)n1] |
| | Halide-4; Amide: 6-cyclopropylpyridine-3-carboxamide |
| | HPLC: XBridge Prep Phenyl OBD Column, 19 x 250 mm, 5 μm; 60-80% |
| | MeCN/H₂O (10 mM NH₄HCO₃ + 0.1% NH₄OH) |
| | LCMS: m/z = 505 [M + H]⁺; ¹H NMR (400 MHz, DMSO-d₆) δ: 9.79 (s, 1H), 9.08 |
| | (s, 1H), 8.90 (d, 1H), 8.63 (s, 1H), 8.27 (s, 1H), 8.11 (dd, 1H), 7.51 (s, 1H), 7.46 |
| | (dd, 1H), 7.36-7.29 (m, 2H), 7.21 (m, 3H), 4.25 (t, 2H), 4.08 (s, 3H), 2.22 (m, 1H), |
| | 1.95 (m, 2H), 1.24 (s, 1H), 1.09-1.00 (m, 4H), 0.96 (m, 3H). |
| 302 | N-(7-(difluoromethoxy)-4-(1-ethyl-3-phenyl-1H-pyrazol-4-yl)quinazolin-6- |
| | yl)cyclopropanecarboxamide |
| | 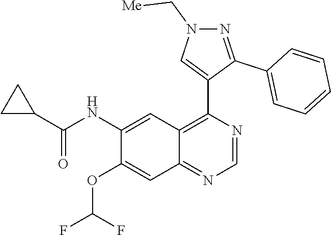 [SMILES: CCn1cc(-c2ncnc3cc(OC(F)F)c(NC(=O)C4CC4)cc23)c(-c2ccccc2)n1] |
| | Halide-5; Amide: cyclopropanecarboxamide |
| | HPLC: YMC-Actus Triart C18 ExRS, 30 x 150 mm, 5 μm; 32-65% MeCN/H₂O (10 |
| | mM NH₄HCO₃ + 0.1% NH₄OH) |
| | White solid (48.9 mg, 44%); LCMS: m/z = 450 [M + H]⁺; ¹H NMR (300 MHz, |
| | DMSO-d₆) δ: 9.94 (s, 1H), 9.13 (s, 1H), 8.75 (s, 1H), 8.29 (s, 1H), 7.97-7.34 (m, |
| | 2H), 7.35-7.26 (m, 2H), 7.22 (d, 3H), 4.32 (q, 2H), 2.20-2.10 (s, 1H), 1.52 (t, 3H), |
| | 0.82 (d, 4H). |
| 303 | N-(7-(2-(dimethylamino)ethoxy)-4-(1-methyl-3-phenyl-1H-pyrazol-4- |
| | yl)quinazolin-6-yl)-4-methylthiazole-5-carboxamide |
| | 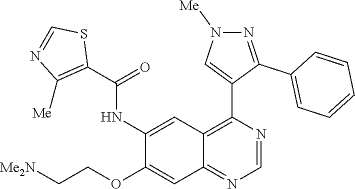 [SMILES: Cc1ncsc1C(=O)Nc1cc2c(-c3cn(C)nc3-c3ccccc3)ncnc2cc1OCCN(C)C] |
| | Halide-3; RCONH₂: 4-methylthiazole-5-carboxamide (Preparation 71). |
| | HPLC: XBridge Prep OBD C18, 30 x 150 mm, 5 μm; 22-42% MeCN/H₂O (10 mM |
| | NH₄HCO₃ + 0.1% NH₄OH) |
| | White solid (22 mg, 32%). LCMS: m/z = 514 [M + H]⁺; ¹H NMR (300 MHz, |
| | DMSO-d₆) δ:9.42 (s, 1H), 9.18 (s, 1H), 9.09 (s, 1H), 8.69 (s, 1H), 8.24 (s, 1H), 7.57 |
| | (s, 1H), 7.34-7.24 (m, 2H), 7.30-7.15 (m, 3H), 4.39 (t, 2H), 4.05 (s, 3H), 2.73 (d, |
| | 5H), 2.23 (s, 6H). |
| 304 | N-(4-(3-(4-fluorophenyl)-1-methyl-1H-pyrazol-4-yl)-7-methoxyquinazolin-6-yl)-4- |
| | methylthiazole-5-carboxamide |
| | 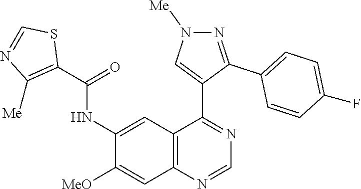 [SMILES: COc1cc2ncnc(-c3cn(C)nc3-c3ccc(F)cc3)c2cc1NC(=O)c1scnc1C] |
| | Halide-6; Amide: 4-methylthiazole-5-carboxamide (Preparation 71) |
| | HPLC: XBridge Shield RP18 OBD Column, 30 x 150 mm, 5 μm; 23-59% |
| | MeCN/H₂O (10 mM NH₄HCO₃ + 0.1% NH₄OH) |
| | Off-white solid (21.9 mg, 64%); LCMS: m/z = 475 [M + H]⁺; ¹H NMR (300 MHz, |
| | DMSO-d₆) δ: 9.52 (s, 1H), 9.13 (s, 1H), 9.07 (s, 1H), 8.53 (s, 1H), 8.22 (s, 1H), |
| | 7.49 (s, 1H), 7.38-7.25 (m, 2H), 7.09-6.96 (m, 2H), 4.03 (d, 6H), 2.64 (s, 3H). |
| 305 | N-(4-(3-(4-fluorophenyl)-1-methyl-1H-pyrazol-4-yl)-7-methoxyquinazolin-6-yl)- |
| | 1,3-dimethyl-1H-pyrazole-4-carboxamide |
| | 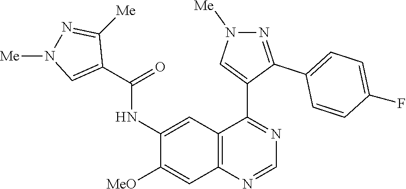 [SMILES: COc1cc2ncnc(-c3cn(C)nc3-c3ccc(F)cc3)c2cc1NC(=O)c1cn(C)nc1C] |
| | Halide-6; Amide: 1,3-dimethyl-1H-pyrazole-4-carboxamide (Preparation 72) |
| | XBridge Shield RP18 OBD Column, 30 x 150 mm, 5 μm; 23-59% MeCN/H₂O (10 |
| | mM NH₄HCO₃ + 0.1% NH₄OH) |
| | White solid (10.1 mg); LCMS: m/z = 472 [M + H]⁺; ¹H NMR (300 MHz, DMSO-d₆) |
| | δ: 8.94 (s, 1H), 8.80 (s, 1H), 8.52 (s, 1H), 8.23 (s, 1H), 8.11 (s, 1H), 7.36 (s, 1H), |
| | 7.27-7.16 (m, 2H), 6.92 (t, 2H), 3.94 (d, 6H), 3.69 (s, 3H), 2.24 (s, 3H), 1.11 (t, |
| | 2H). |
| 306^{B} | N-(7-ethoxy-4-(1-methyl-3-phenyl-1H-pyrazol-4-yl)quinazolin-6- |
| | yl)cyclopropanecarboxamide |
| | 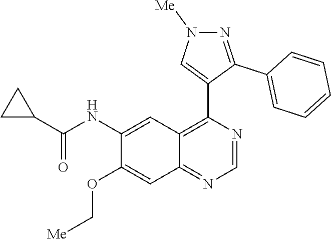 [SMILES: CCOc1cc2ncnc(-c3cn(C)nc3-c3ccccc3)c2cc1NC(=O)C1CC1] |
| | Halide-1; RCONH₂: cyclopropanecarboxamide. |
| | HPLC (XBridge Shield RP18 OBD, 30 x 150 mm, 5 μm; 32-43% MeCN/H₂O (10 |
| | mM NH₄HCO₃ + 0.1% NH₄OH) |
| | White solid (61.9 mg, 60%). LCMS: m/z = 414 [M + H]⁺; ¹H NMR (400 MHz, |
| | DMSO-d₆) δ: 9.51 (s, 1H), 9.02 (s, 1H), 8.68 (s, 1H), 8.16 (s, 1H), 7.42 (s, 1H), |
| | 7.29-7.22 (m, 2H), 7.22-7.15 (m, 3H), 4.37 (q, 2H), 4.02 (s, 3H), 2.17-2.09 (m, |
| | 1H), 1.50 (t, 3H), 1.49 (s, 1H), 0.80 (d, 4H). |
| 307 | N-(4-(1-ethyl-3-phenyl-1H-pyrazol-4-yl)-7-methoxyquinazolin-6-yl)-1- |
| | (trifluoromethyl)-1H-pyrazole-4-carboxamide |
| | 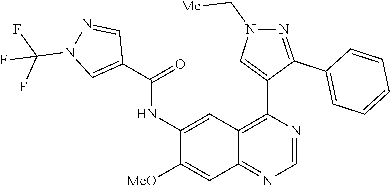 [SMILES: CCn1cc(-c2ncnc3cc(OC)c(NC(=O)c4cnn(C(F)(F)F)c4)cc23)c(-c2ccccc2)n1] |
| | Halide-8; Amide: 1-(trifluoromethyl)pyrazole-4-carboxamide |
| | HPLC: Xbridge Shield RP18 OBD, 30 x 150 mm, 5 μm; 8-56% MeCN/H₂O (10 |
| | mM NH₄HCO₃ + 0.1% NH₄OH) |
| | Off-white solid (33.7 mg, 27%); LCMS: m/z = 508 [M + H]⁺; ¹H NMR (300 MHz, |
| | DMSO-d₆) δ: 9.69 (s, 1H), 9.22 (s, 1H), 9.09 (s, 1H), 8.54 (s, 1H), 8.44 (s, 1H), |
| | 8.27 (s, 1H), 7.52 (s, 1H), 7.37-7.26 (m, 2H), 7.21 (dp, 3H), 4.32 (q, 2H), 4.09 (s, |
| | 3H), 1.53 (t, 3H). |
| 308 | N-(4-(1-(2-(dimethylamino)ethyl)-3-phenyl-1H-pyrazol-4-yl)-7- |
| | methoxyquinazolin-6-yl)bicyclo[1.1.1]pentane-1-carboxamide |
| | 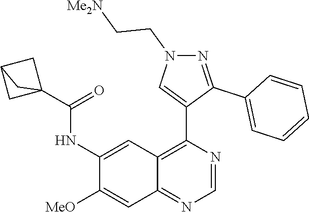 [SMILES: COc1cc2ncnc(-c3cn(CCN(C)C)nc3-c3ccccc3)c2cc1NC(=O)C12CC(C1)C2] |
| | Halide-9: RCO₂H: bicyclo[1.1.1]pentane-1-carboxamide (Preparation 70) |
| | HPLC: Atlantis HILIC OBD, 19 x 150 mm, 5 μm; 47-57% MeCN/H₂O (10 mM |
| | NH₄HCO₃ + 0.1% NH₄OH) |
| | White solid (33.7 mg, 32%); LCMS: m/z = 425 [M + H]⁺; ¹H NMR (400 MHz, |
| | DMSO-d₆) δ: 9.05 (s, 1H), 8.75 (s, 1H), 8.58 (s, 1H), 8.24 (s, 1H), 7.47 (s, 1H), |
| | 7.30 (m, 2H), 7.22 (m, 3H), 4.37 (t, 2H), 4.08 (s, 3H), 2.80 (t, 2H), 2.48 (s, 1H), |
| | 2.24 (s, 6H), 2.08 (s, 5H). |
| 309^{B} | N-(7-methoxy-4-(1-methyl-3-phenyl-1H-pyrazol-4-yl)pyrido[3,2-d]pyrimidin-6- |
| | yl)bicyclo[1.1.1]pentane-1-carboxamide |
| | 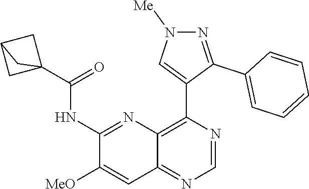 [SMILES: COc1cc2ncnc(-c3cn(C)nc3-c3ccccc3)c2nc1NC(=O)C12CC(C1)C2] |
| | Halide-2; RCONH₂: bicyclo[1.1.1]pentane-1-carboxamide (Preparation 70). |
| | HPLC: XBridge Shield RP18 OBD Column, 30 x 150 mm, 5 μm; 8-56% |
| | MeCN/H₂O (10 mM NH₄HCO₃ + 0.1% NH₄OH) |
| | White solid (43.5 mg, 36%). LCMS: m/z = 495 [M + H]⁺; ¹H NMR (300 MHz, |
| | DMSO-d₆) δ: 9.56 (s, 1H), 9.41 (s, 1H), 8.84 (s, 1H), 7.74 (s, 1H), 7.57-7.48 (m, |
| | 2H), 7.34 (dd, 3H), 4.10 (s, 3H), 4.00 (s, 3H), 3.19 (s, 1H), 2.19 (s, 6H). |
| 310^{B} | 1-isopropyl-N-(7-methoxy-4-(1-methyl-3-phenyl-1H-pyrazol-4-yl)pyrido[3,2- |
| | d]pyrimidin-6-yl)-3-methyl-1H-pyrazole-4-carboxamide |
| | 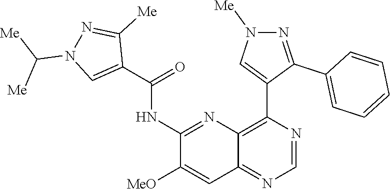 [SMILES: COc1cc2ncnc(-c3cn(C)nc3-c3ccccc3)c2nc1NC(=O)c1cn(C(C)C)nc1C] |
| | Halide-2; RCONH₂: 1-isopropyl-3-methyl-1H-pyrazole-4-carboxamide |
| | (Preparation 77). |
| | HPLC: YMC-Actus Triart C18 ExRS, 30 x 150 mm, 5 μm; 30-50% MeCN/H₂O (10 |
| | mM NH₄HCO₃ + 0.1% NH₄OH) |
| | White solid (12.9 mg, 13%). LCMS: m/z = 483 [M + H]⁺; ¹H NMR (300 MHz, |
| | DMSO-d₆) δ: 9.89 (s, 1H), 9.37 (s, 1H), 8.85 (s, 1H), 8.55 (s, 1H), 7.74 (s, 1H), |
| | 7.54 (td, 2H), 7.34 (qd, 3H), 4.47 (h, 1H), 4.10 (s, 3H), 4.00 (s, 3H), 2.47 (s, 3H), |
| | 1.45 (d, 6H). |
| 311^{A,B} | N-(7-ethoxy-4-(1-methyl-3-phenyl-1H-pyrazol-4-yl)pyrido[3,2-d]pyrimidin-6- |
| | yl)bicyclo[1.1.1]pentane-1-carboxamide |
| | 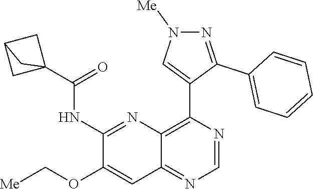 [SMILES: CCOc1cc2ncnc(-c3cn(C)nc3-c3ccccc3)c2nc1NC(=O)C12CC(C1)C2] |
| | Halide-7; RCONH₂: bicyclo[1.1.1]pentane-1-carboxamide (Preparation 70) |
| | HPLC: XBridge Shield RP18 OBD, 30 x 150 mm, 5 μm; 34-62% MeCN/H₂O (10 |
| | mM NH₄HCO₃ + 0.1% NH₄OH) |
| | White solid (22.5 mg, 23%). LCMS: m/z = 441 [M + H]⁺; ¹H NMR (300 MHz, |
| | DMSO-d₆) δ: 9.41 (d, 2H), 8.83 (s, 1H), 7.71 (s, 1H), 7.57-7.48 (m, 2H), 7.34 (dd, |
| | 3H), 4.37 (q, 2H), 4.00 (s, 3H), 2.54 (s, 1H), 2.18 (s, 6H), 1.48 (t, 3H). |
| 312 | 4-fluoro-N-(7-methoxy-4-(1-methyl-3-phenyl-1H-pyrazol-4-yl)pyrido[3,2- |
| | d]pyrimidin-6-yl)benzamide |
| | 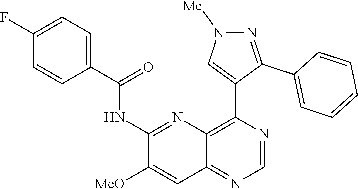 [SMILES: COc1cc2ncnc(-c3cn(C)nc3-c3ccccc3)c2nc1NC(=O)c1ccc(F)cc1] |
| | Halide-2; RCONH₂: 4-fluorobenzamide |
| | HPLC: YMC-Actus Triart C18 ExRS, 30 x 150 mm, 5 μm; 30-50% MeCN/H₂O (10 |
| | mM NH₄HCO₃ + 0.1% NH₄OH) |
| | Off-white solid (4.7 mg, 3.6%). LCMS: m/z = 455 [M + H]⁺; ¹H NMR (300 MHz, |
| | DMSO-d₆) δ: 10.59 (s, 1H), 9.18 (s, 1H), 8.89 (s, 1H), 8.17-8.07 (m, 2H), 7.80 (s, |
| | 1H), 7.51 (dd, 2H), 7.42 (t, 2H), 7.33 (p, 3H), 4.08 (s, 3H), 3.99 (s, 3H). |
| 313 | 3-fluoro-N-(7-methoxy-4-(1-methyl-3-phenyl-1H-pyrazol-4-yl)pyrido[3,2- |
| | d]pyrimidin-6-yl)benzamide |
| | 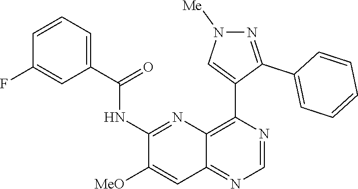 [SMILES: COc1cc2ncnc(-c3cn(C)nc3-c3ccccc3)c2nc1NC(=O)c1cccc(F)c1] |
| | Halide-2; RCONH₂: 3-fluorobenzamide |
| | HPLC: XBridge Shield RP18 OBD, 30 x 150 mm, 5 μm; 8-56% MeCN/H₂O (10 |
| | mM NH₄HCO₃ + 0.1% NH₄OH) |
| | Off-white solid (11.7 mg, 9%). LCMS: m/z = 455 [M + H]⁺; ¹H NMR (300 MHz, |
| | DMSO-d₆) δ: 10.63 (s, 1H), 9.16 (s, 1H), 8.90 (s, 1H), 7.87 (dd, 2H), 7.81 (s, 1H), |
| | 7.64 (td, 1H), 7.52 (td, 3H), 7.37-7.29 (m, 3H), 4.08 (s, 3H), 4.00 (s, 3H). |
| 314 | N-(4-(1-ethyl-3-phenyl-1H-pyrazol-4-yl)-7-methoxypyrido[3,2-d]pyrimidin-6-yl)- |
| | 1-(trifluoromethyl)-1H-pyrazole-4-carboxamide |
| | 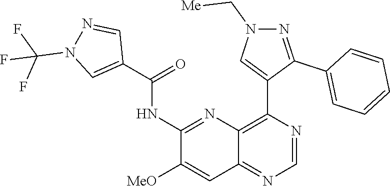 [SMILES: CCn1cc(-c2ncnc3cc(OC)c(NC(=O)c4cnn(C(F)(F)F)c4)nc23)c(-c2ccccc2)n1] |
| | Halide-10; RCONH₂: 1-(trifluoromethyl)pyrazole-4-carboxamide |
| | YMC-Actus Triart C18 ExRS, 30 x 150 mm, 5 μm; 30-50% MeCN/H₂O (10 mM |
| | NH₄HCO₃ + 0.1% NH₄OH) |
| | Off-white solid (9.9 mg, 12%). LCMS: m/z = 509 [M + H]⁺; ¹H NMR (300 MHz, |
| | DMSO-d₆) δ: 10.55 (s, 1H), 9.30 (d, 2H), 8.89 (s, 1H), 8.54 (d, 1H), 7.81 (s, 1H), |
| | 7.58-7.49 (m, 2H), 7.33 (dt, 3H), 4.29 (q, 2H), 4.11 (s, 3H), 1.56 (t, 3H). |
| 315 | N-(4-(1-ethyl-3-phenyl-1H-pyrazol-4-yl)-7-methoxypyrido[3,2-d]pyrimidin-6- |
| | yl)bicyclo[1.1.1]pentane-1-carboxamide |
| | 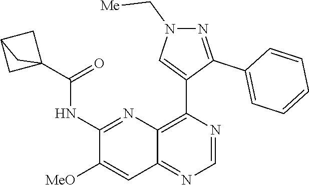 [SMILES: CCn1cc(-c2ncnc3cc(OC)c(NC(=O)C45CC(C4)C5)nc23)c(-c2ccccc2)n1] |
| | Halide-10; RCONH₂: bicyclo[1.1.1]pentane-1-carboxamide (Preparation 70) |
| | HPLC: YMC-Actus Triart C18 ExRS, 30 x 150 mm, 5 μm; 30-50% MeCN/H₂O (10 |
| | mM NH₄HCO₃ + 0.1% NH₄OH) |
| | Off-white solid (16.4 mg, 23%). LCMS: m/z = 441 [M + H]⁺; ¹H NMR (300 MHz, |
| | DMSO-d₆) δ: 9.60 (s, 1H), 9.46 (s, 1H), 8.76 (s, 1H), 7.76 (s, 1H), 7.60-7.50 (m, |
| | 2H), 7.34 (dd, 3H), 4.28 (q, 2H), 4.10 (s, 3H), 3.16 (s, 1H), 2.16 (s, 6H), 1.57 (t, |
| | 3H). |
| 316 | N-(7-methoxy-4-(1-methyl-3-phenyl-1H-pyrazol-4-yl)pyrido[3,2-d]pyrimidin-6- |
| | yl)-2-methyltetrahydrofuran-2-carboxamide |
| | 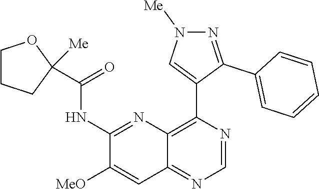 [SMILES: COc1cc2ncnc(-c3cn(C)nc3-c3ccccc3)c2nc1NC(=O)C1(C)CCCO1] |
| | Halide-2; RCONH₂: 2-methyloxolane-2-carboxamide |
| | Column chromatography (SiO₂): 20:1 DCM/MeOH |
| | Off-white solid (60 mg, 22%). LCMS: m/z = 445 [M + H]⁺; ¹H NMR (300 MHz, |
| | DMSO-d₆) δ: 10.06 (s, 1H), 9.77 (s, 1H), 8.80 (s, 1H), 7.73 (s, 1H), 7.60-7.48 (m, |
| | 2H), 7.35 (qd, 3H), 4.13 (s, 3H), 4.09-4.00 (m, 2H), 4.01 (s, 3H), 2.46-2.31 (m, |
| | 1H), 2.08-1.86 (m, 3H), 1.51 (s, 3H). |
| 317 | N-(4-(3-(2-fluorophenyl)-1-methyl-1H-pyrazol-4-yl)-7-methoxypyrido[3,2- |
| | d]pyrimidin-6-yl)-1-(trifluoromethyl)cyclopropane-1-carboxamide |
| | 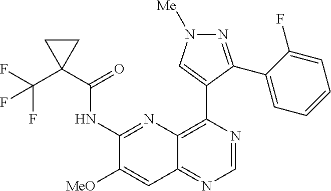 [SMILES: COc1cc2ncnc(-c3cn(C)nc3-c3ccccc3F)c2nc1NC(=O)C1(C(F)(F)F)CC1] |
| | Halide-11; RCONH₂: 1-(trifluoromethyl)cyclopropane-1-carboxamide |
| | HPLC: XBridge Shield RP18 OBD, 30 x 150 mm, 5 μm; 22-65% MeCN/H₂O (10 |
| | mM NH₄HCO₃ + 0.1% NH₄OH) |
| | Yellow solid (12.8 mg, 10%). LCMS: m/z = 487 [M + H]⁺; ¹H NMR (400 MHz, |
| | DMSO-d₆) δ: 9.72 (s, 1H), 9.39 (s, 1H), 8.79 (s, 1H), 8.33 (s, 1H), 7.75 (s, 1H), |
| | 7.54-7.50 (m, 1H), 7.45-7.39 (m, 1H), 7.27-7.22 (m, 1H), 7.17-7.11 (m, 1H), 4.09 |
| | (s, 3H), 4.02 (s, 3H), 1.64 (m, 2H), 1.47 (m, 2H). |
| 318 | 2-cyclopropyl-N-(7-methoxy-4-(1-methyl-3-phenyl-1H-pyrazol-4-yl)pyrido[3,2- |
| | d]pyrimidin-6-yl)thiazole-5-carboxamide |
| | 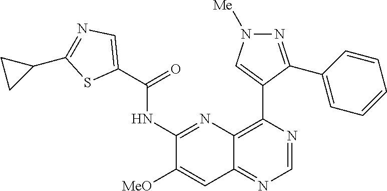 [SMILES: COc1cc2ncnc(-c3cn(C)nc3-c3ccccc3)c2nc1NC(=O)c1cnc(C2CC2)s1] |
| | Halide-2; RCONH₂: 2-cyclopropyl-1,3-thiazole-5-carboxamide |
| | HPLC: XSelect CSH C18, 30 x 150 mm, 5 μm; 37-52% MeCN/H₂O (MeCN/H₂O |
| | (10 mM NH₄HCO₃ + 0.1% NH₄OH) |
| | Yellow solid (29.8 mg, 22%). LCMS: m/z = 484 [M + H]⁺; ¹H NMR (300 MHz, |
| | DMSO-d₆) δ: 10.66 (s, 1H), 9.06 (s, 1H), 8.90 (s, 1H), 8.51 (s, 1H), 7.79 (s , 1H), |
| | 7.52-7.48 (m, 2H), 7.35-7.28 (m, 3H), 4.07 (s, 3H), 3.98 (s, 3H), 2.56-2.50 (m, 1H), |
| | 1.26-1.19 (m, 2H), 1.12-1.06 (m, 2H). |
| 319^{A} | 1-(2-(dimethylamino)ethyl)-N-(4-(1-ethyl-3-phenyl-1H-pyrazol-4-yl)-7- |
| | methoxypyrido[3,2-d]pyrimidin-6-yl)-1H-pyrazole-4-carboxamide |
| | 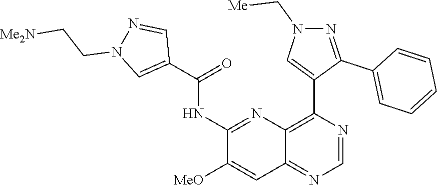 [SMILES: CCn1cc(-c2ncnc3cc(OC)c(NC(=O)c4cnn(CCN(C)C)c4)nc23)c(-c2ccccc2)n1] |
| | Halide-10; RCONH₂: 1-(2-(dimethylamino)ethyl)-1H-pyrazole-4-carboxamide |
| | (Preparation 62) |
| | HPLC: XBridge Shield RP18 OBD, 30 x 150 mm, 5 μm; 7-56% MeCN/H₂O (10 |
| | mM NH₄HCO₃ + 0.1% NH₄OH) |
| | White solid (45.5 mg, 32%). LCMS: m/z = 512 [M + H]⁺; ¹H NMR (300 MHz, |
| | DMSO-d₆) δ: 10.07 (s, 1H), 9.35 (s, 1H), 8.85 (s, 1H), 8.51 (s, 1H), 8.13 (s, 1H), |
| | 7.75 (s, 1H), 7.53 (dd, 2H), 7.33 (dd, 3H), 4.37-4.19 (m, 4H), 4.09 (s, 3H), 2.68 (t, |
| | 2H), 2.18 (s, 6H), 1.56 (t, 3H). |

^{A}toluene was used in place of dioxane
^{B}Cs₂CO₃ was used in place of K₂CO₃

### Example 320: N-(7-methoxy-4-(1-(methyl-d₃)-3-phenyl-1H-pyrazol-4-yl)pyrido[3,2-d]pyrimidin-6-yl)-1-(trifluoromethyl)cyclopropane-1-carboxamide

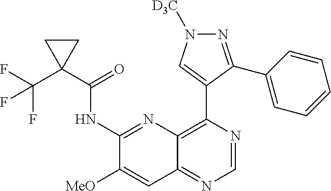 [SMILES: [2H]C([2H])([2H])n1cc(-c2ncnc3cc(OC)c(NC(=O)C4(C(F)(F)F)CC4)nc23)c(-c2ccccc2)n1]

A mixture of XPhos (64.8 mg, 0.112 mmol), Pd₂(dba)₃ (58.3 mg, 56.3 μmol), Cs₂CO₃ (275 mg, 0.844 mmol), 2-cyclopropyl-3,3,3-trifluoropropanamide (141 mg, 0.844 mmol) and a mixture of 6-chloro-7-methoxy-4-(1-(methyl-d₃)-3-phenyl-1H-pyrazol-4-yl)pyrido[3,2-d]pyrimidine and 6-chloro-7-methoxy-4-(1-(methyl-d₃)-5-phenyl-1H-pyrazol-4-yl)pyrido[3,2-d]pyrimidine (Preparation 236, 200 mg, 0.563 mmol) in dioxane (10 mL) was heated at 100° C. for 3 h under N₂. The reaction mixture was diluted with EtOAc (120 mL) and washed with water (60 mL). The combined organics were dried (Na₂SO₄) and evaporated to dryness in vacuo. The residue was purified by prep-TLC (20:1 DCM/MeOH) and by prep-HPLC (XSelect CSH Prep C18 OBD, 19×250 mm, 5 μm; 63-74% MeCN/H₂O (10 mM NH₂HCO₃+0.1% NH₄OH) to afford:

Peak 1, off-white solid (33.5 mg). Example 320: N-(7-methoxy-4-(1-(methyl-d₃)-3-phenyl-1H-pyrazol-4-yl)pyrido[3,2-d]pyrimidin-6-yl)-1-(trifluoromethyl)cyclopropane-1-carboxamide. LCMS: m/z=472 [M+H]⁺; ¹H NMR (400 MHZ, DMSO-d₆) δ: 9.77 (s, 1H), 9.16 (s, 1H), 8.88 (s, 1H), 7.77 (s, 1H), 7.54-7.46 (m, 2H), 7.38-7.28 (m, 3H), 4.08 (s, 3H), 1.62 (s, 2H), 1.54-1.42 (m, 2H).

And Peak 2, N-(7-methoxy-4-(1-(methyl-d₃)-5-phenyl-1H-pyrazol-4-yl)pyrido[3,2-d]pyrimidin-6-yl)-1-(trifluoromethyl)cyclopropane-1-carboxamide.

### Example 321: N-(7-methoxy-4-(1-methyl-3-phenyl-1H-pyrazol-4-yl)quinazolin-6-yl)-5-methylnicotinamide

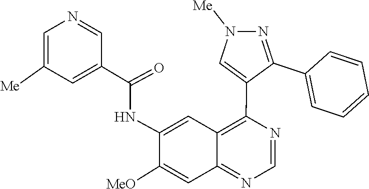 [SMILES: COc1cc2ncnc(-c3cn(C)nc3-c3ccccc3)c2cc1NC(=O)c1cncc(C)c1]

A mixture of EDCl (173 mg, 903 μmol), DIPEA (37.0 mg, 301 μmol), 7-methoxy-4-(1-methyl-3-phenyl-1H-pyrazol-4-yl)quinazolin-6-amine (Preparation 190, 100 mg, 301 μmol) and 5-methylpyridine-3-carboxylic acid (123 mg, 903 μmol) in DCM (5 mL) was stirred at rt for 2 h. The reaction mixture was diluted with EtOAc (100 mL) and washed sequentially with water (3×100 mL) and saturated brine (100 mL). The combined organics were dried (Na₂SO₄) and evaporated to dryness in vacuo. The residue was sequentially purified by prep-TLC (20:1 DCM/MeOH) and prep-HPLC (XBridge Shield RP18 OBD Column, 30×150 mm, 5 μm; 25-40% MeCN/H₂O (10 mM NH₄HCO₃+0.1% NH₄OH)) to afford the title compound as a white solid (86.8 mg, 64.2%). LCMS: m/z=451 [M+H]⁺; ¹H NMR (300 MHz, DMSO-d₆) δ: 9.92 (s, 1H), 9.09 (s, 1H), 8.87 (d, 1H), 8.65-8.58 (m, 1H), 8.56 (s, 1H), 8.23 (s, 1H), 8.09 (d, 1H), 7.52 (s, 1H), 7.35-7.17 (m, 5H), 4.05 (m, 6H), 2.39 (s, 3H).

### Example 322: N-(7-methoxy-4-(1-methyl-3-phenyl-1H-pyrazol-4-yl)quinazolin-6-yl)propionamide

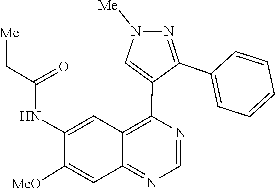 [SMILES: CCC(=O)Nc1cc2c(-c3cn(C)nc3-c3ccccc3)ncnc2cc1OC]

Propanoyl chloride (26.7 mg, 289 μmol) was added to 7-methoxy-4-(1-methyl-3-phenyl-1H-pyrazol-4-yl)quinazolin-6-amine (Preparation 190, 80 mg, 241 μmol) and TEA (48.6 mg, 482 μmol) in DCM (4 mL) at 0° C. and stirred at rt for 1 h. The reaction mixture was evaporated to dryness and the residue purified by prep-HPLC (XBridge Prep OBD C18 Column, 30×150 mm, 5 μm; 35-65% MeCN/H₂O (10 mmol/L NH₄HCO₃+0.1% NH₃·H₂O) to afford the title compound as a white solid (41.8 mg, 45%). LCMS: m/z=388 [M+H]⁺; ¹H NMR (300 MHz, DMSO-d₆) δ: 9.00 (s, 1H), 8.71 (d, 1H), 8.15 (s, 1H), 7.41 (s, 1H), 7.26 (dd, 2H), 7.20-7.13 (m, 3H), 4.02 (d, 6H), 2.44-2.33 (m, 2H), 1.02 (t, 3H).

### Example 323: N-(7-methoxy-4-(1-methyl-3-phenyl-1H-pyrazol-4-yl)quinazolin-6-yl)-6-methylnicotinamide

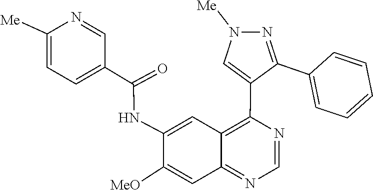 [SMILES: COc1cc2ncnc(-c3cn(C)nc3-c3ccccc3)c2cc1NC(=O)c1ccc(C)nc1]

A solution of EDCl (173 mg, 903 μmol), DMAP (111 mg, 903 μmol), 7-methoxy-4-(1-methyl-3-phenyl-1H-pyrazol-4-yl)quinazolin-6-amine (Preparation 190, 100 mg, 301 μmol) and 6-methylpyridine-3-carboxylic acid (123 mg, 903 μmol) in DCM (5 mL) was stirred at rt for 2 h. The reaction mixture was diluted with EtOAc (100 mL) and washed sequentially with water (3×100 mL) and saturated brine (100 mL). The combined organics were dried (Na₂SO₄) and evaporated to dryness. The residue was purified by prep-TLC (20:1 DCM/MeOH) followed by prep-HPLC (Xselect CSH C18 OBD, 30×150 mm, 5 μm, 20-35% MeCN/H₂O (0.1% HCO₂H)) to afford the title compound as a white solid (53.7 mg, 40%). LCMS: m/z=451 [M+H]⁺; ¹H NMR (300 MHz, DMSO-d₆) δ: 9.87 (s, 1H), 9.09 (s, 1H), 8.96 (d, 1H), 8.55 (s, 1H), 8.23 (s, 1H), 8.16 (dd, 1H), 7.52 (s, 1H), 7.42 (d, 1H), 7.35-7.27 (m, 2H), 7.21 (ddt, 3H), 4.10 (s, 3H), 4.05 (s, 3H), 2.56 (s, 3H).

### Example 324-325

The title compounds were prepared from the 7-methoxy-4-(1-methyl-3-phenyl-1H-pyrazol-4-yl)quinazolin-6-amine (Preparation 190) and the appropriate carboxylic acid (RCO₂H) using an analogous method to that described for Example 323.

| Example | |
|---|---|
| Number | Name/Structure/RCO₂H/Data |
| 324 | 5-fluoro-N-(7-methoxy-4-(1-methyl-3-phenyl-1H-pyrazol-4-yl)quinazolin-6- |
| | yl)picolinamide |
| | 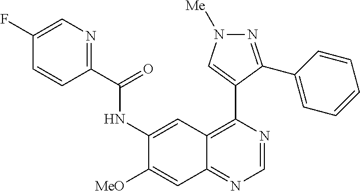 [SMILES: COc1cc2ncnc(-c3cn(C)nc3-c3ccccc3)c2cc1NC(=O)c1ccc(F)cn1] |
| | RCO₂H: 5-fluoropyridine-2-carboxylic acid |
| | HPLC: YMC-Actus Triart C18 ExRS, 30 × 150 mm, 5 μm; 34-67% MeCN/H₂O (10 |
| | mM NH₄HCO₃ + 0.1% NH₄OH) |
| | Pink solid (33.2 mg, 24%). LCMS: m/z = 455 [M + H]⁺; ¹H NMR (400 MHz, |
| | DMSO-d₆) δ: 10.48 (s, 1H), 9.07 (s, 2H), 8.78-8.73 (m, 1H), 8.26 (d, 2H), 8.05- |
| | 7.97 (m, 1H), 7.54 (s, 1H), 7.35-7.28 (m, 2H), 7.18 (d, 3H), 4.16 (s, 6H). |
| 325 | N-(7-methoxy-4-(1-methyl-3-phenyl-1H-pyrazol-4-yl)quinazolin-6- |
| | yl)nicotinamide |
| | 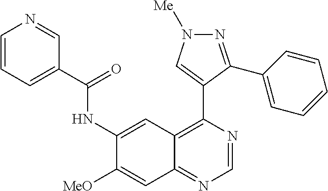 [SMILES: COc1cc2ncnc(-c3cn(C)nc3-c3ccccc3)c2cc1NC(=O)c1cccnc1] |
| | RCO₂H: pyridine-3-carboxylic acid |
| | HPLC: XBridge Shield RP18 OBD, 30 × 150 mm, 5 μm; 20-40% MeCN/H₂O (10 |
| | mM NH₄HCO₃ + 0.1% NH₄OH) |
| | White solid (127 mg, 64%). LCMS: m/z = 437 [M + H]⁺; ¹H NMR (300 MHz, |
| | DMSO-d₆) δ: 9.98 (s, 1H), 9.13-9.03 (m, 2H), 8.78 (dd, 1H), 8.56 (s, 1H), 8.31- |
| | 8.20 (m, 2H), 7.57 (ddd, 1H), 7.52 (s, 1H), 7.31 (ddt, 2H), 7.21 (dp, 3H), 4.06 (d, |
| | 6H). |

### Example 326: 4-fluoro-N-(7-methoxy-4-(1-methyl-3-phenyl-1H-pyrazol-4-yl)quinazolin-6-yl) nicotinamide

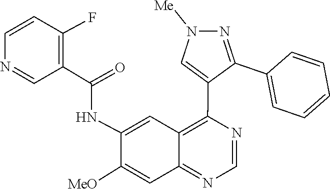 [SMILES: COc1cc2ncnc(-c3cn(C)nc3-c3ccccc3)c2cc1NC(=O)c1cnccc1F]

HATU (400 mg, 1.05 mmol) and DIPEA (156 mg, 1.20 mmol) was added to 7-methoxy-4-(1-methyl-3-phenyl-1H-pyrazol-4-yl)quinazolin-6-amine (Preparation 190, 100 mg, 301 μmol) and 4-fluoropyridine-3-carboxylic acid (127 mg, 903 μmol) in DMF (10 mL) and the mixture stirred at 50° C. for 3 h. The reaction mixture was diluted with EtOAc (120 mL) and washed with water (60 mL). The organic layer was dried (Na₂SO₄) and evaporated to dryness and the residue purified by prep-TLC (10:1 DCM/MeOH) followed by prep-HPLC (YMC-Actus Triart C18, 30×150 mm, 5 μm; 25-62% MeCN/H₂O (10 mM NH₄HCO₃+0.1% NH₄OH) to afford the title compound as an off-white solid (49.8 mg, 35%). LCMS: m/z=455 [M+H]+; ¹H NMR (400 MHZ, DMSO-d₆) δ: 10.10 (s, 1H), 9.09 (s, 1H), 8.81-8.72 (m, 2H), 8.62-8.56 (m, 1H), 8.23 (s, 1H), 7.69 (t, 1H), 7.50 (s, 1H), 7.29 (qd, 2H), 7.25-7.16 (m, 3H), 4.05 (d, 6H).

### Example 327-331

The title compounds were prepared from 7-methoxy-4-(1-methyl-3-phenyl-1H-pyrazol-4-yl) quinazolin-6-amine (Preparation 190) and the appropriate carboxylic acid (RCO₂H) using an analogous method to that described for Example 326.

| Example | |
|---|---|
| Number | Name/Structure/RCO₂H/Data |
| 327 | 5-fluoro-N-(7-methoxy-4-(1-methyl-3-phenyl-1H-pyrazol-4-yl)quinazolin-6- |
| | yl)nicotinamide |
| | 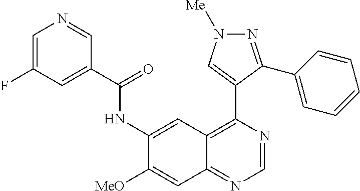 [SMILES: COc1cc2ncnc(-c3cn(C)nc3-c3ccccc3)c2cc1NC(=O)c1cncc(F)c1] |
| | RCO₂H: 5-fluoronicotinic acid |
| | HPLC: XBridge Shield RP18 OBD, 30 × 150 mm, 5 μm; 27-40% MeCN/H₂O (10 |
| | mM NH₄HCO₃ + 0.1% NH₄OH) |
| | Off-white solid (45.7 mg, 33%). LCMS: m/z = 455 [M + H]⁺; ¹H NMR (400 MHz, |
| | DMSO-d₆) δ: 10.06 (s, 1H), 9.10 (s, 1H), 8.94 (t, 1H), 8.80 (d, 1H), 8.53 (s, 1H), |
| | 8.23-8.13 (m, 2H), 7.52 (s, 1H), 7.36-7.27 (m, 2H), 7.21 (tt, 3H), 4.07 (s, 6H). |
| 328 | N-(7-methoxy-4-(1-methyl-3-phenyl-1H-pyrazol-4-yl)quinazolin-6-yl)pyridazine- |
| | 4-carboxamide |
| | 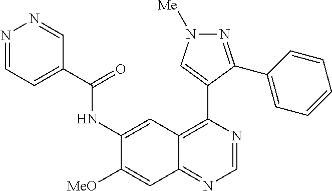 [SMILES: COc1cc2ncnc(-c3cn(C)nc3-c3ccccc3)c2cc1NC(=O)c1ccnnc1] |
| | RCO₂H: pyridazine-4-carboxamide |
| | HPLC: XBridge Shield RP18 OBD, 30 × 150 mm, 5 μm; 11-41% MeCN/H₂O (10 |
| | mM NH₄HCO₃ + 0.1% NH₄OH) |
| | Pale-yellow solid (54.4 mg, 52%). LCMS: m/z = 438 [M + H]⁺; ¹H NMR (300 MHz, |
| | DMSO-d₆) δ: 10.31 (s, 1H), 9.54 (dd, 1H), 9.46 (dd, 1H), 9.09 (s, 1H), 8.54 (s, 1H), |
| | 8.20 (s, 1H), 8.03 (dd, 1H), 7.51 (s, 1H), 7.33-7.23 (m, 2H), 7.19 (dt, 3H), 4.05 (d, |
| | 3H) ,4.01 (s, 3H). |
| 329 | N-(7-methoxy-4-(1-methyl-3-phenyl-1H-pyrazol-4-yl)quinazolin-6-yl)-6- |
| | methylpicolinamide |
| | 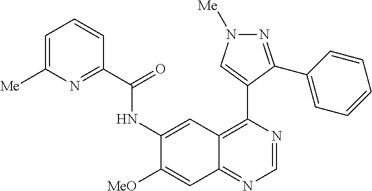 [SMILES: COc1cc2ncnc(-c3cn(C)nc3-c3ccccc3)c2cc1NC(=O)c1cccc(C)n1] |
| | RCO₂H: 6-methylpyridine-2-carboxylic acid |
| | HPLC: XBridge Shield RP18 OBD, 30 × 150 mm, 5 μm; 52-77% MeCN/H₂O (10 |
| | mM NH₄HCO₃ + 0.1% NH₄OH) |
| | White solid (5.1 mg, 4%). LCMS: m/z = 451 [M + H]⁺; ¹H NMR (300 MHz, |
| | DMSO-d₆) δ: 10.83 (s, 1H), 7.06 (d, 2H), 8.26 (s, 1H), 7.98 (d, 2H), 7.57 (d, 2H), |
| | 7.30 (d, 2H), 7.18 (m, 3H), 4.18 (s, 3H), 4.06 (s, 3H), 2.73 (s, 3H). |
| 330 | 3-fluoro-N-(7-methoxy-4-(1-methyl-3-phenyl-1H-pyrazol-4-yl)quinazolin-6-yl)-6- |
| | methylpicolinamide |
| | 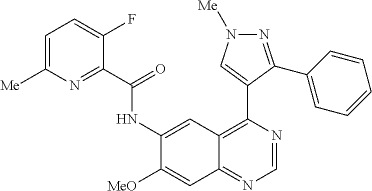 [SMILES: COc1cc2ncnc(-c3cn(C)nc3-c3ccccc3)c2cc1NC(=O)c1nc(C)ccc1F] |
| | RCO₂H: 3-fluoro-6-methylpyridine-2-carboxylic acid |
| | HPLC: YMC-Actus Triart C18 ExRS, 30 × 150 mm, 5 μm; 33-66% MeCN/H₂O (10 |
| | mM NH₄HCO₃ + 0.1% NH₄OH) |
| | Yellow solid (49.1 mg, 35%). LCMS: m/z = 469 [M + H]⁺; ¹H NMR (400 MHz, |
| | DMSO-d₆) δ: 10.65 (s, 1H), 9.07 (s, 1H), 8.97 (s, 1H), 8.24 (s, 1H), 7.85 (dd, 1H), |
| | 7.65 (dd, 1H), 7.53 (s, 1H), 7.35-7.26 (m, 2H), 7.19 (qd, 3H), 4.16 (s, 3H), 4.06 (s, |
| | 3H), 2.59 (s, 3H). |
| 331 | 2,6-difluoro-N-(7-methoxy-4-(1-methyl-3-phenyl-1H-pyrazol-4-yl)quinazolin-6- |
| | yl)benzamide |
| | 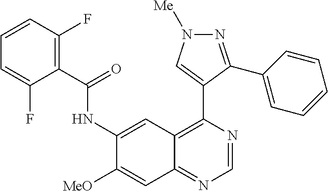 [SMILES: COc1cc2ncnc(-c3cn(C)nc3-c3ccccc3)c2cc1NC(=O)c1c(F)cccc1F] |
| | RCO₂H: 2,6-difluorobenzoic acid |
| | HPLC: XBridge Prep OBD C18, 30 × 150 mm, 5 μm; 26-56% MeCN/H₂O (10 mM |
| | NH₄HCO₃ + 0.1% NH₄OH) |
| | Yellow solid (7.5 mg, 5%). LCMS: m/z = 472 [M + H]⁺; ¹H NMR (300 MHz, |
| | DMSO-d₆) δ: 10.38 (s, 1H), 9.09 (s, 1H), 8.77 (s, 1H),8.25 (s, 1H), 7.56-7.54(m, |
| | 1H), 7.47 (s, 1H), 7.26-7.17 (m, 7H), 4.03 (s, 6H). |

### Example 332: N-(4-(1-ethyl-3-phenyl-1H-pyrazol-4-yl)-7-methoxyquinazolin-6-yl) nicotinamide

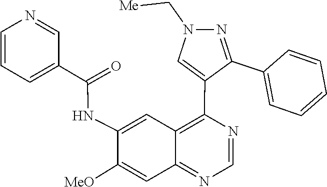 [SMILES: CCn1cc(-c2ncnc3cc(OC)c(NC(=O)c4cccnc4)cc23)c(-c2ccccc2)n1]

Nicotinoyl chloride (122 mg, 0.868 mmol) was added to TEA (132 mg, 1.30 mmol) and 4-(1-ethyl-3-phenyl-1H-pyrazol-4-yl)-7-methoxyquinazolin-6-amine (Preparation 252, 150 mg, 0.434 mmol) in THF (10 mL) at 0° C. and the resulting mixture was heated to rt for 3 h. The reaction mixture was diluted with EtOAc (120 mL) and washed with water (60 mL). The combined organics were dried (Na₂SO₄) and evaporated to dryness in vacuo. The residue was purified by prep-HPLC (XBridge Shield RP18 OBD Column, 30×150 mm, 5 μm; 4-52% MeCN/H₂O (10 mM NH₄HCO₃+0.1% NH₄OH)) to afford the title compound as a white solid (125 mg, 64%). LCMS: m/z=451 [M+H]⁺; ¹H NMR (400 MHZ, DMSO-d₆) δ: 9.96 (s, 1H), 9.12-9.04 (m, 2H), 8.77 (dd, 1H), 8.61 (s, 1H), 8.31-8.22 (m, 2H), 7.56 (dd, 2H), 7.38-7.29 (m, 2H), 7.21 (q, 3H), 4.33 (q, 2H), 4.08 (s, 3H), 1.53 (t, 3H).

### Example 333: 6-cyclopropyl-N-(4-(1-(2,2-difluoroethyl)-3-phenyl-1H-pyrazol-4-yl)-7-methoxyquinazolin-6-yl) nicotinamide

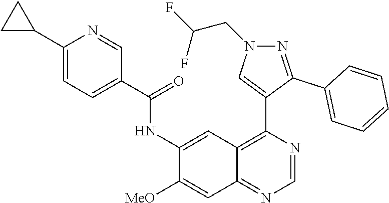 [SMILES: COc1cc2ncnc(-c3cn(CC(F)F)nc3-c3ccccc3)c2cc1NC(=O)c1ccc(C2CC2)nc1]

To a mixture of 4-(1-(2,2-difluoroethyl)-3-phenyl-1H-pyrazol-4-yl)-7-methoxypyrido[3,2-d]pyrimidin-6-amine trifluoroaceate (Preparation 223, 100 mg, 0.2622 mmol) in DCM (5 mL) was added 6-cyclopropylpyridine-3-carbonyl chloride (Part 1, Example 229, 47.6 mg, 0.2622 mmol) and pyridine (62.1 mg, 0.78 mmol) and the reaction mixture was stirred at rt for 3 h. The reaction mixture was concentrated under vacuum. The crude product was purified by Prep-HPLC (XBridge Prep OBD C18 Column: YMC-Actus Triart C18 ExRS, 30 mm×150 mm, 5 μm; Mobile Phase A: Water (10 mmol/L NH₄HCO₃+0.1% NH₃·H₂O), Mobile Phase B: MeCN; Flow rate: 60 mL/min; Gradient: 34% B to 67% B in 7 min, to give the title compound as a white solid, 26 mg 18.8%. LCMS: m/z=427 [M+H]⁺; ¹H NMR (300 MHz, DMSO-d₆) δ: 9.80 (s, 1H), 9.11 (s, 1H), 8.90 (d, 1H), 8.54 (s, 1H), 8.33 (s, 1H), 8.11 (dd, 1H), 7.54-7.43 (m, 2H), 7.31 (dd, 2H), 7.27-7.15 (m, 3H), 6.54 (t, 2H), 4.96-4.72 (m, 2H), 4.08 (s, 3H), 2.22 (s, 1H), 1.12-0.95 (m, 4H).

### Example 334 and 335: (1R,5R)—N-(4-(1-ethyl-3-(4-fluorophenyl)-1H-pyrazol-4-yl)-7-methoxyquinazolin-6-yl)-3-oxabicyclo[3.1.0]hexane-1-carboxamide and (1S,5S)—N-(4-(1-ethyl-3-(4-fluorophenyl)-1H-pyrazol-4-yl)-7-methoxyquinazolin-6-yl)-3-oxabicyclo[3.1.0]hexane-1-carboxamide

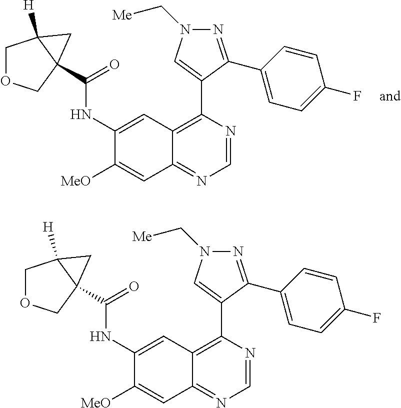 [SMILES: CCn1cc(-c2ncnc3cc(OC)c(NC(=O)[C@@]45COC[C@@H]4C5)cc23)c(-c2ccc(F)cc2)n1 ; CCn1cc(-c2ncnc3cc(OC)c(NC(=O)[C@]45COC[C@H]4C5)cc23)c(-c2ccc(F)cc2)n1]

Part 1. To a solution of 4-(1-ethyl-3-(4-fluorophenyl)-1H-pyrazol-4-yl)-7-methoxyquinazolin-6-amine (Preparation 255, 80 mg, 0.22 mmol) in THF was added 3-oxabicyclo[3.1.0]hexane-1-carboxylic acid (84.5 mg, 0.660 mmol), T3P® (0.5 mL) and pyridine (0.5 mL) and the mixture stirred at 60° C. for 3 h. The reaction mixture was evaporated to dryness in vacuo and the residue purified by prep-HPLC (XBridge Shield RP18 OBD Column, 30×150 mm, 5 μm; 11-57% MeCN/H₂O (10 mM NH₄HCO₃+0.1% NH₄OH)) to give N-(4-(1-ethyl-3-(4-fluorophenyl)-1H-pyrazol-4-yl)-7-methoxyquinazolin-6-yl)-3-oxabicyclo[3.1.0]hexane-1-carboxamide as a white solid (40 mg, 38%).

Part 2. N-(4-(1-ethyl-3-(4-fluorophenyl)-1H-pyrazol-4-yl)-7-methoxyquinazolin-6-yl)-3-oxabicyclo[3.1.0]hexane-1-carboxamide (Part 1, 40 mg, 84.5 μmol) in MeOH was purified by chiral-HPLC (Chiral Art Cellulose-SB, 20×250 mm, 5 μm; 5% MeOH/(50% Hex/MTBE (0.5% 2M NH₃—MeOH))) to afford:

Peak 1, white solid (12.4 mg). Example 334: (1R,5R)—N-(4-(1-ethyl-3-(4-fluorophenyl)-1H-pyrazol-4-yl)-7-methoxyquinazolin-6-yl)-3-oxabicyclo[3.1.0]hexane-1-carboxamide or (1S,5S)—N-(4-(1-ethyl-3-(4-fluorophenyl)-1H-pyrazol-4-yl)-7-methoxyquinazolin-6-yl)-3-oxabicyclo[3.1.0]hexane-1-carboxamide. LCMS: m/z=474 [M+H]⁺; ¹H NMR (300 MHz, DMSO-d₆) δ: 8.94 (s, 1H), 8.58 (s, 1H), 8.33 (s, 1H), 8.13 (s, 1H), 7.34 (s, 1H), 7.30-7.17 (m, 2H), 7.02-6.88 (m, 2H), 4.19 (q, 2H), 3.94 (s, 3H), 3.83 (s, 2H), 3.68-3.54 (m, 2H), 2.04 (ddd, 1H), 1.39 (t, 3H), 1.26 (dd, 1H), 0.76 (t, 1H).

Peak 2, white solid (9.1 mg). Example 335: (1S,5S)—N-(4-(1-ethyl-3-(4-fluorophenyl)-1H-pyrazol-4-yl)-7-methoxyquinazolin-6-yl)-3-oxabicyclo[3.1.0]hexane-1-carboxamide or (1R,5R)—N-(4-(1-ethyl-3-(4-fluorophenyl)-1H-pyrazol-4-yl)-7-methoxyquinazolin-6-yl)-3-oxabicyclo[3.1.0]hexane-1-carboxamide. LCMS: m/z=474 [M+H]⁺; ¹H NMR (300 MHz, DMSO-d₆) δ: 8.94 (s, 1H), 8.58 (s, 1H), 8.33 (s, 1H), 8.13 (s, 1H), 7.34 (s, 1H), 7.30-7.17 (m, 2H), 7.02-6.88 (m, 2H), 4.19 (q, 2H), 3.94 (s, 3H), 3.83 (s, 2H), 3.68-3.54 (m, 2H), 2.04 (ddd, 1H), 1.39 (t, 3H), 1.26 (dd, 1H), 0.76 (t, 1H).

### Example 336 and 337: (1S,5S)—N-(7-methoxy-4-(1-methyl-3-phenyl-1H-pyrazol-4-yl)quinazolin-6-yl)-3-methyl-3-azabicyclo[3.1.0]hexane-1-carboxamide and (1R,5R)—N-(7-methoxy-4-(1-methyl-3-phenyl-1H-pyrazol-4-yl)quinazolin-6-yl)-3-methyl-3-azabicyclo[3.1.0]hexane-1-carboxamide

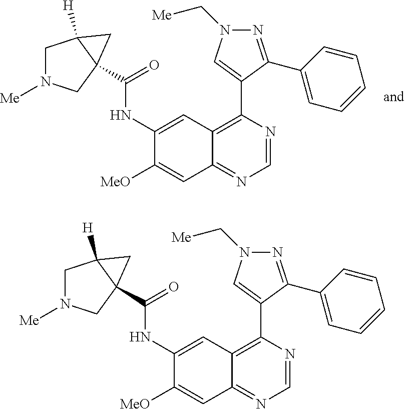 [SMILES: CCn1cc(-c2ncnc3cc(OC)c(NC(=O)[C@@]45C[C@@H]4CN(C)C5)cc23)c(-c2ccccc2)n1 ; CCn1cc(-c2ncnc3cc(OC)c(NC(=O)[C@]45C[C@H]4CN(C)C5)cc23)c(-c2ccccc2)n1]

Part 1. The mixture of tert-butyl 1-((7-methoxy-4-(1-methyl-3-phenyl-1H-pyrazol-4-yl)quinazolin-6-yl)carbamoyl)-3-azabicyclo[3.1.0]hexane-3-carboxylate (Preparation 257, 310 mg, 0.573 mmol) in TFA (1 mL) and DCM (2 mL) was stirred at 20° C. for 2 hr. The reaction mixture was extracted with EtOAc (3×20 mL), washed with brine (10 mL), dried (Na₂SO₄) and evaporated to dryness. The residue was purified by prep-TLC (10:1 DCM/MeOH) to afford N-(7-methoxy-4-(1-methyl-3-phenyl-1H-pyrazol-4-yl)quinazolin-6-yl)-3-azabicyclo[3.1.0]hexane-1-carboxamide as a yellow solid (200 mg, 79%). LCMS: m/z=441 [M+H]⁺.

Part 2. To a mixture of N-(7-methoxy-4-(1-methyl-3-phenyl-1H-pyrazol-4-yl)quinazolin-6-yl)-3-azabicyclo[3.1.0]hexane-1-carboxamide (Part 1, 200 mg, 454 μmol) in MeOH (3 mL) was added HCHO (184 mg, 2.27 mmol, 37% purity) and the mixture was stirred at 20° C. for 20 min. NaBH(OAc)₃ (288 mg, 1.36 μmol) was added and the mixture was stirred at 20° C. for 1 hr. and then evaporated to dryness. The residue was purified by prep-HPLC (YMC-Actus Triart C18 ExRS, 30×150 mm, 5 μm; 17-50% MeCN/H₂O (10 mM NH₄HCO₃+0.1% NH₄OH)) to afford N-(7-methoxy-4-(1-methyl-3-phenyl-1H-pyrazol-4-yl)quinazolin-6-yl)-3-methyl-3-azabicyclo[3.1.0]hexane-1-carboxamide as a white solid (120 mg, 58%) which was subsequently purified by chiral-SFC (Reg-AD, 30×250 mm, 5 μm; 25% IPA (0.5% 2M NH₃—MeOH) in CO₂) to afford:

Peak 1, white solid (26.1 mg). Example 336: (1S,5S)—N-(7-methoxy-4-(1-methyl-3-phenyl-1H-pyrazol-4-yl)quinazolin-6-yl)-3-methyl-3-azabicyclo[3.1.0]hexane-1-carboxamide or (1R,5R)—N-(7-methoxy-4-(1-methyl-3-phenyl-1H-pyrazol-4-yl)quinazolin-6-yl)-3-methyl-3-azabicyclo[3.1.0]hexane-1-carboxamide. LCMS: m/z=429 [M+H]⁺; ¹H NMR (400 MHZ, DMSO-d₆) δ: 9.05 (s, 1H), 8.53 (d, 2H), 8.17 (s, 1H), 7.47 (s, 1H), 7.32-7.24 (m, 2H), 7.24-7.16 (m, 3H), 4.08 (s, 3H), 4.02 (s, 3H), 3.18 (d, 1H), 2.90 (d, 1H), 2.57 (d, 1H), 2.33 (dd, 1H), 2.27 (s, 3H), 1.93 (ddd, 1H), 1.30 (t, 1H), 1.16 (dd, 1H).

Peak 2, white solid (34.1 mg). Example 337: (1R,5R)—N-(7-methoxy-4-(1-methyl-3-phenyl-1H-pyrazol-4-yl)quinazolin-6-yl)-3-methyl-3-azabicyclo[3.1.0]hexane-1-carboxamide or (1S,5S)—N-(7-methoxy-4-(1-methyl-3-phenyl-1H-pyrazol-4-yl)quinazolin-6-yl)-3-methyl-3-azabicyclo[3.1.0]hexane-1-carboxamide. LCMS: m/z=429 [M+H]⁺; ¹H NMR (400 MHZ, DMSO-d₆) δ: 9.05 (s, 1H), 8.53 (d, 2H), 8.17 (s, 1H), 7.48 (s, 1H), 7.31-7.24 (m, 2H), 7.21 (q, 3H), 4.08 (s, 3H), 4.02 (s, 3H), 3.19 (d, 1H), 2.90 (d, 1H), 2.58 (d, 1H), 2.34 (d, 1H), 2.28 (s, 3H), 1.98-1.89 (m, 1H), 1.30 (t, 1H), 1.16 (dd, 1H).

### Example 338: N-(4-(3-(2,6-difluorophenyl)-1-methyl-1H-pyrazol-4-yl)-7-methoxyquinazolin-6-yl)cyclopropanecarboxamide

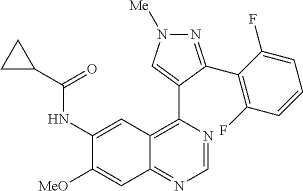 [SMILES: COc1cc2ncnc(-c3cn(C)nc3-c3c(F)cccc3F)c2cc1NC(=O)C1CC1]

To the mixture of N-(4-chloro-7-methoxyquinazolin-6-yl)cyclopropanecarboxamide (Preparation 201, 50 mg, 0.180 mmol), potassium carbonate (49.8 mg, 0.360 mmol) and 3-(2,6-difluorophenyl)-1-methyl-1H-pyrazole (Preparation 7, 72.1 mg, 0.225 mmol) in DME (0.4 mL), dioxane (0.7 mL) and H₂O (0.2 mL) was added Pd(PPh₃)₄ (20.8 mg, 0.018 mmol) and the mixture purged with N₂ and stirred at 100° C. for 1 h. The reaction mixture was filtered through a celite plug with DCM/MeOH and the filtrate evaporated to dryness in vacuo and the residue purified by ISCO (12 g SiO₂, 0-10% MeOH/DCM) to afford the title compound as a pale-yellow solid (58 mg, 74%). LCMS: m/z=429 [M+H]⁺; ¹H NMR (500 MHZ, DMSO-d₆) δ: 9.79 (d, 1H), 8.90 (t, 1H), 8.84 (d, 1H), 8.41 (d, 1H), 7.43 (d, 1H), 7.16-6.94 (m, 2H), 4.08 (dd, 5H), 2.22 (s, 1H), 0.86 (dd, 4H).

### Example 339: trans-rac-(3R,4R)-3-fluoro-N-(7-methoxy-4-(1-methyl-3-phenyl-1H-pyrazol-4-yl)quinazolin-6-yl)-1-methylpiperidine-4-carboxamide trifluoroacetate

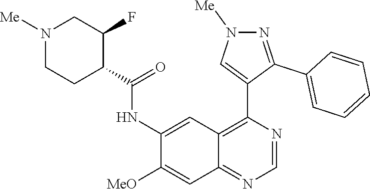 [SMILES: COc1cc2ncnc(-c3cn(C)nc3-c3ccccc3)c2cc1NC(=O)[C@@H]1CCN(C)C[C@H]1F]

Part 1. To a solution of trans-rac-(3R,4R)-1-(tert-butoxycarbonyl)-3-fluoropiperidine-4-carboxylic acid (107 mg, 0.433 mmol) in pyridine (5 mL) was added EDCl (89 mg, 0.462 mmol) and 7-methoxy-4-(1-methyl-3-phenyl-1H-pyrazol-4-yl)quinazolin-6-amine (Preparation 190, 95.7 mg, 0.289 mmol) and the resulting mixture stirred at rt overnight. The reaction mixture was evaporated to dryness and the residue purified by chromatography (SiO₂, ISCO, 0-100% 10% MeOH (+0.1% NH₄Cl)/DCM) to afford trans-rac-tert-butyl (3R,4R)-3-fluoro-4-((7-methoxy-4-(1-methyl-3-phenyl-1H-pyrazol-4-yl)quinazolin-6-yl)carbamoyl) piperidine-1-carboxylate (42.4 mg, 26.2%) which was treated with TFA (1 mL). The mixture was evaporated to dryness to afford trans-rac-(3R,4R)-3-fluoro-N-(7-methoxy-4-(1-methyl-3-phenyl-1H-pyrazol-4-yl)quinazolin-6-yl) piperidine-4-carboxamide which was used without purification.

Part 2. In a 20 mL vial was placed trans-rac-(3R,4R)-3-fluoro-N-(7-methoxy-4-(1-methyl-3-phenyl-1H-pyrazol-4-yl)quinazolin-6-yl) piperidine-4-carboxamide (Part 1, 35 mg, 0.076 mmol) and formaldehyde solution in THF (2 mL, 37% w/w) and evaporated to dryness. The residue was diluted with THF (0.5 mL) and MeOH (0.5 mL) and formaldehyde (0.5 mL, 6.16 mmol) and gently heated to obtain a clear solution. The solution was cooled to rt and DIPEA (208 μl, 1.192 mmol) added followed by NaBH(OAc)₃ (48.3 mg, 0.228 mmol) after 3 min and the reaction stirred at rt. The mixture was diluted with EtOAc and sat NaHCO₃. Reaction mixture was evaporated and the residue purified by HPLC (Gilson using 1% TFA modified MeCN/H₂O) to afford the title compound (35 mg, 26%). LCMS: m/z=475 [M+H]⁺; ¹H NMR (400 MHZ, MeOH-d₄) δ: 9.08 (d, 1H), 8.86 (s, 1H), 8.20 (s, 1H), 7.48 (d, 1H), 7.31 (d, 2H), 7.21 (q, 3H), 5.20-4.80 (m, 1H), 4.00-3.00 (m, 6H), 4.16 (d, 3H), 4.13 (d, 3H), 3.00 (s, 3H) 2.60-2.00 (m, 2H).

### Example 340: N-(7-methoxy-4-(1-methyl-3-phenyl-1H-pyrazol-4-yl)quinazolin-6-yl)bicyclo[1.1.1]pentane-1-carboxamide

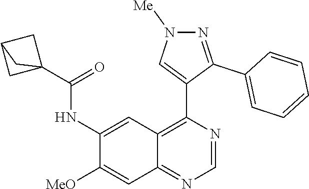 [SMILES: COc1cc2ncnc(-c3cn(C)nc3-c3ccccc3)c2cc1NC(=O)C12CC(C1)C2]

Bicyclo[1.1.1]pentane-1-carboxylic acid (18.37 mg, 0.164 mmol) and EDCl (50.3 mg, 0.262 mmol) were added to a solution of 7-methoxy-4-(1-methyl-3-phenyl-1H-pyrazol-4-yl)quinazolin-6-amine (Preparation 190, 54.3 mg, 0.164 mmol) in pyridine (1.5 mL) and DCM (1.0 mL) and the mixture stirred overnight at rt. The reaction mixture was evaporated to dryness in vacuo and the residue purified by ISCO chromatography (SiO₂, 12 g Redi ISCO: 0-100% (10% MeOH+1% NH₄Cl)/DCM to afford the title compound (44.9 mg, 63%). LCMS: m/z=426 [M+H]⁺; ¹H NMR (400 MHZ, CDCl₃) δ: 9.08 (q, 1H), 8.97 (q, 1H), 7.99 (s, 1H), 7.85 (q, 1H), 7.40 (m, 2H, 7.35 (d, 1H, 7.18 (s, 3H), 4.09 (s, 3H), 4.06 (s, 3H), 2.55 (q, 1H), 2.16 (s, 6H).

### Example 341:3-(dimethylamino)-N-(7-methoxy-4-(1-methyl-3-phenyl-1H-pyrazol-4-yl)quinazolin-6-yl)bicyclo[1.1.1]pentane-1-carboxamide trifluoroacetate

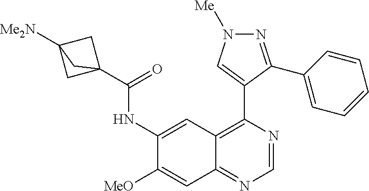 [SMILES: COc1cc2ncnc(-c3cn(C)nc3-c3ccccc3)c2cc1NC(=O)C12CC(N(C)C)(C1)C2]

To a solution of 3-(dimethylamino) bicyclo[1.1.1]pentane-1-carboxylic acid (14.05 mg, 0.091 mmol) in Pyridine (1.5 mL) was added EDCl (18.51 mg, 0.097 mmol) and 7-methoxy-4-(1-methyl-3-phenyl-1H-pyrazol-4-yl)quinazolin-6-amine (Preparation 190, 20 mg, 0.060 mmol) and the mixture stirred at rt for 3 h. The reaction was evaporated to dryness in vacuo and the residue purified by HPLC (Gilson with 0.01% TFA modified MeCN/H₂O) to afford the title compound (30.2 mg, 89%). LCMS: m/z=469 [M+H]⁺; ¹H NMR (DMSO-d₆) δ: 9.14 (s, 1H), 9.08 (s, 1H), 8.48 (s, 1H), 8.18 (s, 1H), 7.49 (s, 1H), 7.30-7.25 (m, 2H), 7.20 (tt, 3H), 4.07 (s, 3H), 4.05 (s, 3H), 2.74 (s, 6H), 2.34 (s, 6H).

### Example 342:3-fluoro-N-(7-methoxy-4-(1-methyl-3-phenyl-1H-pyrazol-4-yl)quinazolin-6-yl)bicyclo[1.1.1]pentane-1-carboxamide

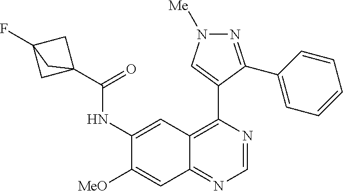 [SMILES: COc1cc2ncnc(-c3cn(C)nc3-c3ccccc3)c2cc1NC(=O)C12CC(F)(C1)C2]

To a solution of 3-fluorobicyclo[1.1.1]pentane-1-carboxylic acid (11.78 mg, 0.091 mmol) in Pyridine (1.5 mL) was added EDCl (18.51 mg, 0.097 mmol) and 7-methoxy-4-(1-methyl-3-phenyl-1H-pyrazol-4-yl)quinazolin-6-amine (Preparation 190, 20 mg, 0.060 mmol) and the mixture stirred at rt for 3 h. The reaction was evaporated to dryness in vacuo and the residue purified by HPLC (Gilson with 0.01% TFA modified MeCN/H₂O) to afford the title compound (26.5 mg, 99%). LCMS: m/z=444 [M+H]⁺; ¹H NMR (DMSO-d₆) δ: 9.07 (s, 1H), 9.04 (s, 1H), 8.49 (s, 1H), 8.19 (s, 1H), 7.48 (s, 1H), 7.28 (dd, 2H), 7.22 (s, OH), 7.24-7.17 (m, 3H), 4.05 (d, 6H), 2.55 (s, 4H), 2.41 (d, 6H).

### Example 343: tert-butyl (4-((7-methoxy-4-(1-methyl-3-phenyl-1H-pyrazol-4-yl)quinazolin-6-yl)carbamoyl)cuban-1-yl)carbamate

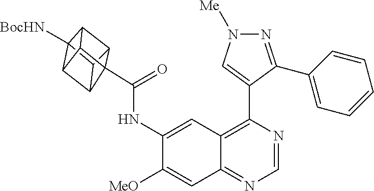 [SMILES: COc1cc2ncnc(-c3cn(C)nc3-c3ccccc3)c2cc1NC(=O)C12C3C4C1C1C2C3C41NC(=O)OC(C)(C)C]

To a solution of 4-((tert-butoxycarbonyl)amino) cubane-1-carboxylic acid (95 mg, 0.362 mmol) in Pyridine (1.5 mL) was added EDCl (93 mg, 0.483 mmol) and 7-methoxy-4-(1-methyl-3-phenyl-1H-pyrazol-4-yl)quinazolin-6-amine (Preparation 190, 100 mg, 0.320 mmol) and the mixture stirred at rt for 1 h. DCM and water and the layers separated. The aqueous layer was reextracted with DCM and the combined organics dried and evaporated to dryness. The residue was purified by chromatography (SiO₂, ISCO, 0-100% EtOAc/DCM followed by 0-25% MeOH/DCM to afford the title compound (141 mg, 81%). ¹H NMR (DMSO-d₆) δ: 9.00 (s, 1H), 8.79 (s, 1H), 8.12 (s, 1H), 7.41 (s, 1H), 7.32-7.30 (m, 2H), 7.30-7.18 (m, 3H), 4.85 (s, 3H), 4.13-4.08 (m, 6H), 1.49 (s, 9H).

### Example 344: N-(7-methoxy-4-(1-methyl-3-phenyl-1H-pyrazol-4-yl)quinazolin-6-yl)cyclopropanecarboxamide

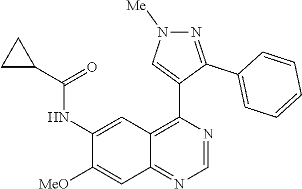 [SMILES: COc1cc2ncnc(-c3cn(C)nc3-c3ccccc3)c2cc1NC(=O)C1CC1]

To a vial containing a solution of 7-methoxy-4-(1-methyl-3-phenyl-1H-pyrazol-4-yl)quinazolin-6-amine (Preparation 190, 25 mg, 0.075 mmol) in THF (0.377 mL) under N₂ was added pyridine (15.25 μL, 0.189 mmol), cyclopropanecarboxylic acid (9.57 μL, 0.113 mmol) and T3P® (112 μl, 0.189 mmol) and the reaction stirred at 50° C. for 3 h. The solvent was removed under reduced pressure and the residue dissolved in DCM and washed with water. The combined extracts were dried (Na₂SO₄) and evaporated to dryness in vacuo. The residue was purified by ISCO (4 g SiO₂, 0-20% MeOH/DCM) followed by RP-ISCO (4-70% MeCN/H₂O (+0.1% TFA) to afford the title compound as a beige solid (16.2 mg, 54%). LCMS: m/z=400 [M+H]⁺; ¹H NMR (500 MHz, DMSO-d₆) δ: 9.69 (s, 1H), 9.03 (d, 1H), 8.69 (s, 1H), 8.16 (s, 1H), 7.44 (s, 1H), 7.26 (d, 2H), 7.19 (d, 3H), 4.08 (s, 3H), 4.02 (s, 3H), 2.15 (s, 1H), 0.78 (d, 4H).

### Example 345-354

The title compounds were prepared from 7-methoxy-4-(1-methyl-3-phenyl-1H-pyrazol-4-yl)quinazolin-6-amine (Preparation 190) and the appropriate carboxylic acid (RCO₂H) using an analogous method to that described for Example 344.

| Example | |
|---|---|
| Number | Name/Structure/RCO₂H/Data |
| 345 | N-(7-methoxy-4-(1-methyl-3-phenyl-1H-pyrazol-4-yl)quinazolin-6-yl)-3- |
| | methyloxetane-3-carboxamide |
| | 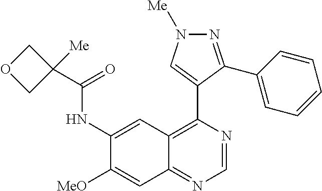 [SMILES: COc1cc2ncnc(-c3cn(C)nc3-c3ccccc3)c2cc1NC(=O)C1(C)COC1] |
| | RCO₂H: 3-methyloxetane-3-carboxylic acid |
| | ISCO (12g, SiO₂; 0-10% MeOH/DCM) |
| | Yellow solid (24.1 mg, 74%). LCMS: m/z = 430 [M + H]⁺; ¹H NMR (500 MHz, |
| | DMSO-d₆) δ: 9.32 (s, 1H), 9.07 (d, 1H), 8.51 (d, 1H), 8.21 (s, 1H), 7.45 (s, 1H), |
| | 7.27 (dd, 2H), 7.23-7.18 (m, 3H), 4.75 (t, 2H), 4.36 (d, 2H), 4.08-4.02 (m, 6H), |
| | 1.58 (s, 3H). |
| 346 | 3,3-difluoro-N-(7-methoxy-4-(1-methyl-3-phenyl-1H-pyrazol-4-yl)quinazolin-6- |
| | yl)cyclobutane-1-carboxamide |
| | 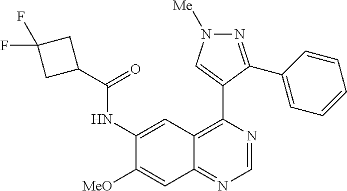 [SMILES: COc1cc2ncnc(-c3cn(C)nc3-c3ccccc3)c2cc1NC(=O)C1CC(F)(F)C1] |
| | RCO₂H: 3,3-difluorocyclobutane-1-carboxylic acid |
| | ISCO (12g, SiO₂; 0-10% MeOH/DCM) |
| | Pink solid (21.7 mg, 64%). LCMS: m/z = 450 [M + H]⁺; ¹H NMR (500 MHz, |
| | DMSO-d₆) δ: 9.63 (s, 1H), 9.07 (d, 1H), 8.75 (s, 1H), 8.21 (d, 1H), 7.45 (d, 1H), |
| | 7.27 (dd, 2H), 7.20 (d, 3H), 4.07 (dd, 6H), 3.37 (d, 1H), 2.76 (dt, 4H). |
| 347 | N-(7-methoxy-4-(1-methyl-3-phenyl-1H-pyrazol-4-yl)quinazolin-6- |
| | yl)cyclobutanecarboxamide |
| | 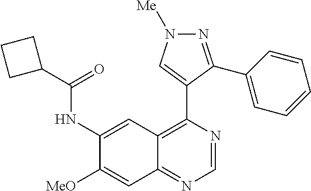 [SMILES: COc1cc2ncnc(-c3cn(C)nc3-c3ccccc3)c2cc1NC(=O)C1CCC1] |
| | RCO₂H: cyclobutanecarboxylic acid |
| | ISCO (12g, SiO₂; 0-10% MeOH/DCM) |
| | Pale green solid (38.8 mg, 89%). LCMS: m/z = 414 [M + H]⁺; ¹H NMR (500 MHz, |
| | DMSO-d₆) δ: 9.15 (d, 1H), 9.09-8.98 (m, 1H), 8.76 (s, 1H), 8.21 (d, 1H), 7.43 (d, |
| | 1H), 7.36-7.13 (m, 5H), 4.06 (d, 6H), 3.50-3.41 (m, 1H), 2.27-2.02 (m, 4H), 2.02- |
| | 1.72 (m, 2H). |
| 348 | N-(7-methoxy-4-(1-methyl-3-phenyl-1H-pyrazol-4-yl)quinazolin-6-y1)-1- |
| | methylcyclobutane-1-carboxamide |
| | 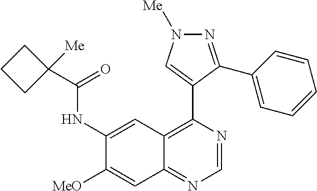 [SMILES: COc1cc2ncnc(-c3cn(C)nc3-c3ccccc3)c2cc1NC(=O)C1(C)CCC1] |
| | RCO₂H: 1-methylcyclobutane-1-carboxylic acid |
| | ISCO (12g, SiO₂; 0-10% MeOH/DCM) |
| | White solid (27.4 mg, 71%). LCMS: m/z = 428 [M + H]⁺; ¹H NMR (500 MHz, |
| | DMSO-d₆) δ: 9.07 (d, 1H), 8.68-8.51 (m, 2H), 8.21 (d, 1H), 7.47 (d, 1H), 7.28 (q, |
| | 2H), 7.21 (q, 3H), 4.07 (dd, 6H), 2.46-2.32 (m, 2H), 1.96 (q, 1H), 1.85 (dq, 2H), |
| | 1.74 (dq, 1H), 1.44 (d, 3H). |
| 349 | N-(7-methoxy-4-(1-methyl-3-phenyl-1H-pyrazol-4-yl)quinazolin-6-yl)-1- |
| | (trifluoromethyl)cyclopropane-1-carboxamide |
| | 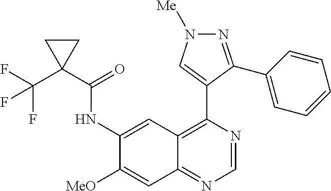 [SMILES: COc1cc2ncnc(-c3cn(C)nc3-c3ccccc3)c2cc1NC(=O)C1(C(F)(F)F)CC1] |
| | RCO₂H: 1-(trifluoromethyl)cyclopropane-1-carboxylic acid |
| | ISCO (12g, SiO₂; 0-10% MeOH/DCM) |
| | White solid (6.6 mg, 16%). LCMS: m/z = 468 [M + H]⁺; ¹H NMR (500 MHz, |
| | DMSO-d₆) δ: 9.10 (d, 1H), 8.98 (s, 1H), 8.46 (d, 1H), 8.21 (d, 1H), 7.51 (d, 1H), |
| | 7.27 (q, 2H), 7.21 (dd, 3H), 4.09 (d, 3H), 4.04 (d, 3H), 1.47 (s, 2H), 1.39 (d, 2H). |
| 350 | 2-fluoro-N-(7-methoxy-4-(1-methyl-3-phenyl-1H-pyrazol-4-yl)quinazolin-6-yl)-2- |
| | methylpropanamide |
| | 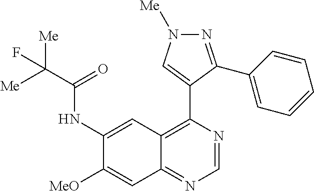 [SMILES: COc1cc2ncnc(-c3cn(C)nc3-c3ccccc3)c2cc1NC(=O)C(C)(C)F] |
| | RCO₂H: 2-fluoro-2-methylpropanoic acid |
| | Product collected by filtration (white solid, 28.1 mg, 74%). LCMS: m/z = 420 |
| | [M + H]⁺; ¹H NMR (500 MHz, DMSO-d₆) δ: 9.10 (d, 1H), 9.02 (d, 1H), 8.70 (d, |
| | 1H), 8.23 (d, 1H), 7.52 (d, 1H), 7.27 (d, 2H), 7.23-7.18 (m, 3H), 4.11 (d, 3H), 4.05 |
| | (d, 3H), 1.60 (d, 3H), 1.56 (d, 3H). |
| 351 | N-(7-methoxy-4-(1-methyl-3-phenyl-1H-pyrazol-4-yl)quinazolin-6- |
| | yl)isonicotinamide |
| | 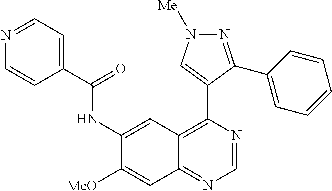 [SMILES: COc1cc2ncnc(-c3cn(C)nc3-c3ccccc3)c2cc1NC(=O)c1ccncc1] |
| | RCO₂H: isonicotinic acid |
| | ISCO (12g, SiO₂; 0-10% MeOH/DCM) and RP-ISCO (5-75% MeCN/H₂O + 0.1% |
| | TFA); LCMS: m/z = 437 [M + H]⁺; ¹H NMR (500 MHz, DMSO-d₆) δ: |
| 352 | 6-(difluoromethyl)-N-(7-methoxy-4-(1-methyl-3-phenyl-1H-pyrazol-4- |
| | yl)quinazolin-6-yl)nicotinamide |
| | 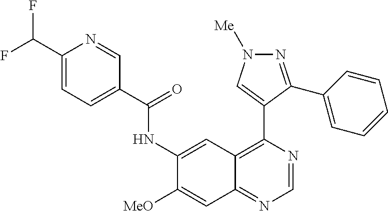 [SMILES: COc1cc2ncnc(-c3cn(C)nc3-c3ccccc3)c2cc1NC(=O)c1ccc(C(F)F)nc1] |
| | RCO₂H: 6-(difluoromethyl)nicotinic acid |
| | ISCO (12g, SiO₂; 0-10% MeOH/DCM) |
| | White solid (29.9 mg, 68%). LCMS: m/z = 487 [M + H]⁺; ¹H NMR (500 MHz, |
| | DMSO-d₆) δ: 10.14 (s, 1H), 9.17-9.08 (m, 2H), 8.58 (s, 1H), 8.44 (d, 1H), 8.24 (d, |
| | 1H), 7.92-7.86 (m, 1H), 7.54 (d, 1H), 7.32 (s, 2H), 7.23 (t, 3H), 7.19-6.93 (m, 1H), |
| | 4.07 (dd, 6H). |
| 353 | N-(4-(1-(2,2-difluoroethyl)-3-phenyl-1H-pyrazol-4-yl)-7-methoxyquinazolin-6- |
| | yl)nicotinamide |
| | 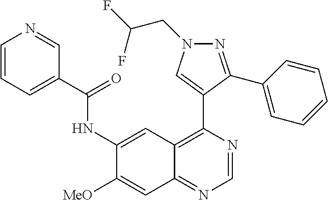 [SMILES: COc1cc2ncnc(-c3cn(CC(F)F)nc3-c3ccccc3)c2cc1NC(=O)c1cccnc1] |
| | RCO₂H: nicotinic acid |
| | ISCO (12g, SiO₂; 0-10% MeOH/DCM) |
| | White solid (24.5 mg, 64%). LCMS: m/z = 487 [M + H]⁺; ¹H NMR (500 MHz, |
| | DMSO-d₆) δ: 9.97 (d, 1H), 9.20-9.09 (m, 1H), 9.07 (s, 1H), 8.79 (d, 1H), 8.60-8.46 |
| | (m, 1H), 8.35 (t, 1H), 8.26 (d, 1H), 7.57 (dd, 2H), 7.40-7.29 (m, 2H), 7.29-7.20 (m, |
| | 3H), 6.75-6.33 (m, 1H), 4.85 (t, 2H), 4.10 (t, 3H). |
| 354 | 2-cyclopropyl-N-(7-methoxy-4-(1-methyl-3-phenyl-1H-pyrazol-4-yl)quinazolin-6- |
| | yl)-4-methylthiazole-5-carboxamide |
| | 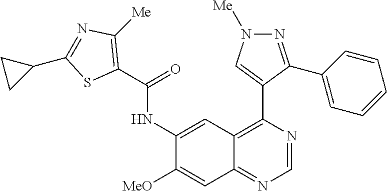 [SMILES: COc1cc2ncnc(-c3cn(C)nc3-c3ccccc3)c2cc1NC(=O)c1sc(C2CC2)nc1C] |
| | RCO₂H: 2-cyclopropyl-4-methylthiazole-5-carboxylic acid |
| | ISCO (12g, SiO₂; 0-10% MeOH/DCM) and RP-ISCO (10-70% MeCN/H₂O + 0.1% |
| | TFA); |
| | White solid (34.3 mg, 33%). LCMS: m/z = 497 [M + H]⁺; ¹H NMR (500 MHz, |
| | DMSO-d₆) δ: 9.27 (s, 1H), 9.09 (s, 1H), 8.60 (s, 1H), 8.23 (s, 1H), 7.52 (s, 1H), |
| | 7.31 (dd, 2H), 7.25-7.15 (m, 3H), 4.09 (s, 3H), 4.05 (s, 3H), 2.58 (s, 3H), 2.44 (tt, |
| | 1H), 1.19 (qd, 2H), 1.09-0.97 (m, 2H). |

### Example 355: N-(7-methoxy-4-(1-methyl-3-phenyl-1H-pyrazol-4-yl)pyrido[3,2-d]pyrimidin-6-yl)cyclopropanecarboxamide

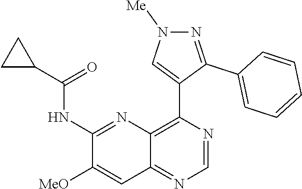 [SMILES: COc1cc2ncnc(-c3cn(C)nc3-c3ccccc3)c2nc1NC(=O)C1CC1]

A mixture of 7-methoxy-4-(1-methyl-3-phenyl-1H-pyrazol-4-yl)pyrido[3,2-d]pyrimidin-6-amine (Preparation 216, 20 mg, 0.060 mmol), cyclopropyl(1H-imidazol-1-yl) methanone (27.29 mg, 0.181 mmol), THF (5 mL), NaH (2.89 mg, 0.120 mmol) under N₂ was stirred for 2 h at 0° C. The reaction was quenched with H₂O (5 mL) and the solids removed by filtration. The filtrate was extracted with EtOAc (3×10 mL) and evaporated to dryness in vacuo. The residue was purified by column chromatography (20:1 DCM/MeOH) follows by prep-HPLC (YMC-Actus Triart C18 ExRS, 30×150 mm, 5 μm; 30-50% MeCN/H₂O (10 mM NH₄HCO₃+0.1% NH₄OH)) to afford the title compound as an off-white solid (4.9 mg, 20%). LCMS: m/z=401 [M+H]⁺; ¹H NMR (400 MHZ, DMSO-d₆) δ: 10.53 (s, 1H), 9.51 (s, 1H), 8.81 (s, 1H), 7.70 (s, 1H), 7.54 (dd, 2H), 7.40-7.29 (m, 3H), 4.11 (s, 3H), 3.97 (s, 3H), 2.31 (dd, 1H), 1.02-0.94 (m, 2H), 0.91 (dt, 2H).

### Example 356-359

The title compounds were prepared from the appropriate pyrido[3,2-d]pyrimidin-6-amine (Amine) and appropriate (1H-imidazol-1-yl) methanone using an analogous method to that described for Example 355.

Amine-1: 7-methoxy-4-(1-methyl-3-phenyl-1H-pyrazol-4-yl)pyrido[3,2-d]pyrimidin-6-amine (Preparation 216); Amine-2:4-(3-(2-fluorophenyl)-1-methyl-1H-pyrazol-4-yl)-7-methoxypyrido[3,2-d]pyrimidin-6-amine (Preparation 221); Amine-3:4-(1-(2,2-difluoroethyl)-3-phenyl-1H-pyrazol-4-yl)-7-methoxypyrido[3,2-d]pyrimidin-6-amine (Preparation 223)

| Example | |
|---|---|
| Number | Name/Structure/Amine/RC(O)imidazole/Data |
| 356 | N-(4-(3-(2-fluorophenyl)-1-methyl-1H-pyrazol-4-yl)-7-methoxypyrido[3,2- |
| | d]pyrimidin-6-yl)cyclopropanecarboxamide |
| | 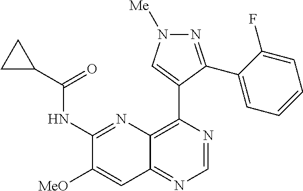 [SMILES: COc1cc2ncnc(-c3cn(C)nc3-c3ccccc3F)c2nc1NC(=O)C1CC1] |
| | Amine-2; RCOIm: cyclopropyl(1H-imidazol-1-yl)methanone |
| | HPLC: YMC-Actus Triart C18 ExRS, 30 × 150 mm, 5 μm; 31-51% MeCN/H₂O (10 |
| | mM NH₄HCO₃ + 0.1% NH₄OH) |
| | Off-white solid (52 mg, 44%). LCMS: m/z = 419 [M + H]⁺; ¹H NMR (300 MHz, |
| | DMSO-d₆) δ: 10.56 (s, 1H), 9.72 (s, 1H), 8.72 (s, 1H), 7.68 (s, 1H), 7.57-7.37 (m, |
| | 2H), 7.25 (td, 1H), 7.17 (dd, 1H), 4.11 (s, 3H), 3.99 (s, 3H), 2.39-2.29 (m, 1H), |
| | 1.05-0.86 (m, 4H). |
| 357 | 6-cyclopropyl-N-(7-methoxy-4-(1-methyl-3-phenyl-1H-pyrazol-4-yl)pyrido[3,2- |
| | d]pyrimidin-6-yl)nicotinamide |
| | 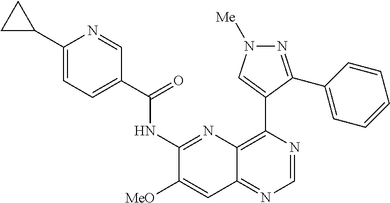 [SMILES: COc1cc2ncnc(-c3cn(C)nc3-c3ccccc3)c2nc1NC(=O)c1ccc(C2CC2)nc1] |
| | Amine-2; RCOIm : (6-cyclopropylpyridin-3-y1)(1H-imidazol-1-yl)methanone |
| | HPLC: XBridge Shield RP18 OBD, 30 × 150 mm, 5 μm; 8-56% MeCN/H₂O (10 |
| | mM NH₄HCO₃ + 0.1% NH₄OH) |
| | Off-white solid (18.4 mg, 26%). LCMS: m/z = 478 [M + H]⁺; ¹H NMR (400 MHz, |
| | DMSO-d₆) δ: 10.65 (s, 1H), 9.17 (d, 1H), 9.00 (d, 1H), 8.88 (d, 1H), 8.21 (dd, 1H), |
| | 7.79 (s, 1H), 7.54-7.46 (m, 3H), 7.32 (dd, 3H), 4.08 (d, 3H), 3.99 (s, 3H), 2.24 (tt, |
| | 1H), 1.11-0.99 (m, 4H). |
| 358 | 6-cyclopropyl-N-(4-(1-(2,2-difluoroethyl)-3-phenyl-1H-pyrazol-4-yl)-7- |
| | methoxypyrido[3,2-d]pyrimidin-6-yl)nicotinamide |
| | 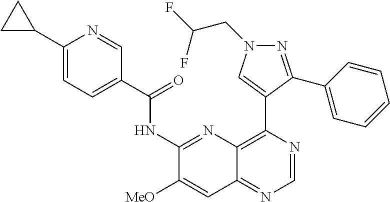 [SMILES: COc1cc2ncnc(-c3cn(CC(F)F)nc3-c3ccccc3)c2nc1NC(=O)c1ccc(C2CC2)nc1] |
| | Amine-3; RCOIm: (6-cyclopropylpyridin-3-yl)(1H-imidazol-1-yl)methanone |
| | HPLC: XBridge Shield RP18 OBD, 30 × 150 mm, 5 μm; 8-56% MeCN/H₂O (10 |
| | mM NH₄HCO₃ + 0.1% NH₄OH) |
| | Off-white solid (14.8 mg, 15%). LCMS: m/z = 528 [M + H]⁺; ¹H NMR (300 MHz, |
| | DMSO-d₆) δ: 10.65 (s, 1H), 9.24 (s, 1H), 9.00 (d, 1H), 8.93 (s, 1H), 8.20 (dd, 1H), |
| | 7.80 (s, 1H), 7.56-7.45 (m, 3H), 7.34 (dp, 3H), 6.54 (t, 1H), 4.77 (td, 2H), 4.08 (s, |
| | 3H), 2.28-2.18 (m, 1H), 1.13-0.99 (m, 4H). |
| 359 | 2-fluoro-N-(7-methoxy-4-(1-methyl-3-phenyl-1H-pyrazol-4-yl)pyrido[3,2- |
| | d]pyrimidin-6-yl)benzamide |
| | 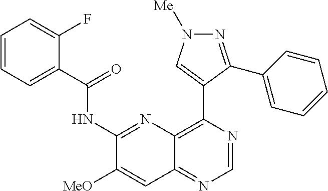 [SMILES: COc1cc2ncnc(-c3cn(C)nc3-c3ccccc3)c2nc1NC(=O)c1ccccc1F] |
| | Amine-1; RCOIm: (2-fluorophenyl)(1H-imidazol-1-yl)methanone |
| | HPLC: XBridge Shield RP18 OBD, 30 × 150 mm, 5 μm; 8-56% MeCN/H₂O (10 |
| | mM NH₄HCO₃ + 0.1% NH₄OH) |
| | Off-white solid (16.3 mg, 17%). LCMS: m/z = 455 [M + H]⁺; ¹H NMR (300 MHz, |
| | DMSO-d₆) δ: 10.62 (d, 1H), 9.35 (d, 1H), 8.84 (s, 1H), 7.95-7.83 (m, 1H), 7.77 (s, |
| | 1H), 7.62 (qd, 1H), 7.51 (dd, 2H), 7.44-7.29 (m, 5H), 4.11 (s, 3H), 4.01 (s, 3H). |

### Example 360: N-(7-methoxy-4-(1-methyl-3-phenyl-1H-pyrazol-4-yl)pyrido[3,2-d]pyrimidin-6-yl)-1-(trifluoromethyl)-1H-pyrazole-4-carboxamide

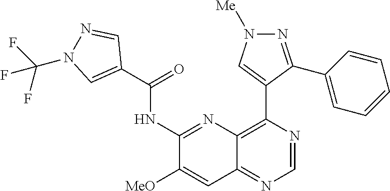 [SMILES: COc1cc2ncnc(-c3cn(C)nc3-c3ccccc3)c2nc1NC(=O)c1cnn(C(F)(F)F)c1]

Into a 20-mL pressure tank reactor purged and maintained with an inert atmosphere of nitrogen was placed 1-(trifluoromethyl)-1H-pyrazole-4-carboxamide (Preparation 76, 30 mg, 0.168 mmol), 6-chloro-7-methoxy-4-(1-methyl-3-phenyl-1H-pyrazol-4-yl)pyrido[3,2-d]pyrimidine (Preparation 242, 70.7 mg, 0.201 mmol), K₂CO₃ (46.6 mg, 0.335 mmol), XPhos (7.99 mg, 0.017 mmol), Pd₂(dba)₃ (15.3 mg, 0.017 mmol) and dioxane (5 mL) and the resulting mixture stirred for 3 h at 100° C. The reaction was quenched by the addition of 5 mL of water and the resulting solution extracted with EtOAc (3×10 mL). The combined organics were evaporated to dryness and the residue purified by column chromatography (20:1 DCM/MeOH) followed by prep-HPLC (XBridge Shield RP18 OBD Column, 30×150 mm, 5 μm; 8-56% MeCN/H₂O (10 mM NH₄HCO₃+0.1% NH₄OH)) to afford the title compound as an off-white solid (6 mg, 7.2%). LCMS: m/z=495 [M+H]⁺; ¹H NMR (300 MHZ, DMSO-d₆) δ: 10.59 (s, 1H), 9.31 (s, 1H), 9.18 (s, 1H), 8.90 (s, 1H), 8.55 (s, 1H), 7.81 (s, 1H), 7.51 (dq, 2H), 7.33 (dt, 3H), 4.10 (s, 3H), 4.00 (s, 3H).

### Example 361: N-(7-methoxy-4-(1-methyl-3-phenyl-1H-pyrazol-4-yl)pyrido[3,2-d]pyrimidin-6-yl)-1-(trifluoromethyl)cyclopropane-1-carboxamide

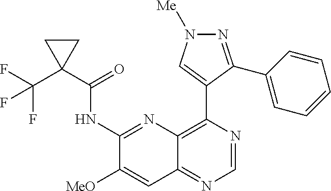 [SMILES: COc1cc2ncnc(-c3cn(C)nc3-c3ccccc3)c2nc1NC(=O)C1(C(F)(F)F)CC1]

Into a 20-mL pressure tank reactor purged and maintained with an inert atmosphere of nitrogen was placed 6-chloro-7-methoxy-4-(1-methyl-3-phenyl-1H-pyrazol-4-yl)pyrido[3,2-d]pyrimidine (Preparation 242, 70 mg, 0.199 mmol), 1-(trifluoromethyl)cyclopropane-1-carboxamide (Preparation 68, 36.6 mg, 0.239 mmol), K₂CO₃ (55.4 mg, 0.398 mmol), Pd₂(dba)₃ (18.2 mg, 0.020 mmol), XPhos (9.49 mg, 0.020 mmol) and dioxane (5 mL) and the resulting solution stirred for 3 h at 100° C. The reaction was quenched by the addition of H₂O (5 mL) and extracted with DCM (3×10 mL). The combined extracts were evaporated to dryness in vacuo and the residue purified by column chromatography (20:1 DCM/MeOH) followed by prep-HPLC Column: YMC-Actus Triart C18 ExRS, 30×150 mm, 5 μm; 30-50% MeCN/H₂O (10 mM NH₄HCO₃+0.1% NH₄OH)) to afford the title compound as a white solid (23.9 mg, 25.6%). LCMS: m/z=469 [M+H]⁺; ¹H NMR (300 MHz, DMSO-d₆) δ: 9.79 (brs, 1H), 9.17 (s, 1H), 8.88 (s, 1H), 7.78 (s, 1H), 7.51-7.48 (m, 2H), 7.33-7.31 (m, 3H), 4.08 (s, 3H), 3.99 (s, 3H), 1.62 (m, 2H), 1.48-1.47 (m, 2H).

### Example 362: N-(4-(3-(4-fluorophenyl)-1-methyl-1H-pyrazol-4-yl)-7-methoxypyrido[3,2-d]pyrimidin-6-yl)-1-(trifluoromethyl)cyclopropane-1-carboxamide

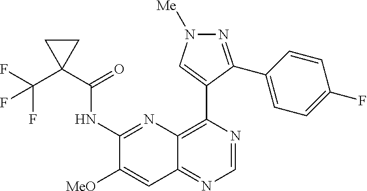 [SMILES: COc1cc2ncnc(-c3cn(C)nc3-c3ccc(F)cc3)c2nc1NC(=O)C1(C(F)(F)F)CC1]

A mixture of 6-chloro-4-(3-(4-fluorophenyl)-1-methyl-1H-pyrazol-4-yl)-7-methoxypyrido[3,2-d]pyrimidine (Preparation 238, 120 mg, 0.324 mmol), 1-(trifluoromethyl)cyclopropane-1-carboxamide (Preparation 68, 59.4 mg, 0.388 mmol), Cs₂CO₃ (158 mg, 0.485 mmol), BINAP Pd G2 (25 mg, 0.027 mmol) in dioxane (10 mL) was stirred at 100° C. for 3 h. The reaction mixture was evaporated to dryness in vacuo and the residue purified by prep-HPLC (YMC-Actus Triart C18 ExRS, 30×150 mm, 5 μm; 46-76% MeCN/H₂O (10 mM NH₄HCO₃+0.1% NH₄OH)) to afford the title compound as a yellow solid (36.7 mg, 23%). LCMS: m/z=487 [M+H]⁺, ¹H NMR (400 MHZ, DMSO-d₆) δ: 9.77 (s, 1H), 9.22 (s, 1H), 8.89 (s, 1H), 7.77 (s, 1H), 7.60-7.50 (m, 2H), 7.21-7.10 (m, 2H), 4.08 (s, 3H), 3.99 (s, 3H), 1.62 (s, 2H), 1.54-1.42 (m, 2H).

### Example 363-371

The title compounds were prepared from the appropriate chloropyrido[3,2-d]pyrimidine (Halide) and amide (RCONH₂) using an analogous method to that described for Example 362.

Halide-A: 6-chloro-7-methoxy-4-(1-methyl-3-phenyl-1H-pyrazol-4-yl)pyrido[3,2-d]pyrimidine (Preparation 242); Halide-B: 6-chloro-4-(1-(difluoromethyl)-3-phenyl-1H-pyrazol-4-yl)-7-methoxypyrido[3,2-d]pyrimidine (Preparation 244); Halide-C: 6-chloro-4-(3-(2-fluorophenyl)-1-methyl-1H-pyrazol-4-yl)-7-methoxypyrido[3,2-d]pyrimidine (Preparation 237); Halide-D: 6-chloro-4-(1-cyclopropyl-3-phenyl-1H-pyrazol-4-yl)-7-methoxypyrido[3,2-d]pyrimidine (Preparation 245); Halide-E: 6-chloro-4-(1-(2,2-difluoroethyl)-3-phenyl-1H-pyrazol-4-yl)-7-methoxypyrido[3,2-d]pyrimidine (Preparation 240); Halide-F: 6-chloro-4-(1-ethyl-3-phenyl-1H-pyrazol-4-yl)-7-methoxypyrido[3,2-d]pyrimidine (Preparation 239)

| Example | |
|---|---|
| Number | Name/Structure/Halide/RCONH₂/Data |
| 363 | (R)-N-(7-methoxy-4-(1-methyl-3-phenyl-1H-pyrazol-4-yl)pyrido[3,2-d]pyrimidin- |
| | 6-yl)-2-methyltetrahydrofuran-2-carboxamide |
| | 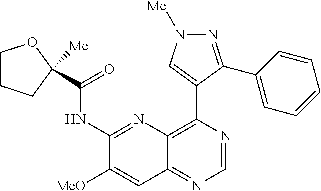 [SMILES: COc1cc2ncnc(-c3cn(C)nc3-c3ccccc3)c2nc1NC(=O)[C@@]1(C)CCCO1] |
| | Halide-A; RCONH₂: (R)-2-methyltetrahydrofuran-2-carboxamide (Preparation |
| | 67) |
| | Prep-TLC: 30:1 DCM/MeOH |
| | White solid (8.1 mg, 9%). LCMS: m/z = 445 [M + H]+; ¹H NMR (300 MHz, |
| | CDCl₃) δ: 10.11 (s, 1H), 9.90 (s, 1H), 8.86 (s, 1H), 7.68-7.54 (m, 3H), 7.37 (dd, |
| | 3H), 4.16-4.03 (m, 8H), 2.59 (t, 1H), 2.10-1.93 (m, 3H), 1.62 (s, 3H). |
| 364 | N-(4-(1-(difluoromethyl)-3-phenyl-1H-pyrazol-4-yl)-7-methoxypyrido[3,2- |
| | d]pyrimidin-6-yl)bicyclo[1.1.1]pentane-1-carboxamide |
| | 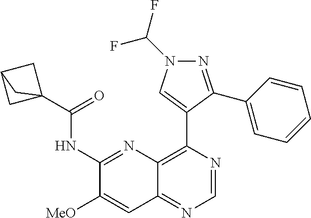 [SMILES: COc1cc2ncnc(-c3cn(C(F)F)nc3-c3ccccc3)c2nc1NC(=O)C12CC(C1)C2] |
| | Halide-B; RCONH₂: bicyclo[1.1.1]pentane-1-carboxamide (Preparation 70) |
| | HPLC: XBridge Shield RP18 OBD, 30 × 150 mm, 5 μm; 23-72% MeCN/H₂O (10 |
| | mM NH₄HCO₃ + 0.1% NH₄OH) |
| | White solid (8.4 mg, 23%). LCMS: m/z = 463 [M + H]⁺; ¹H NMR (300 MHz, |
| | DMSO-d₆) δ: 9.78 (s, 1H), 9.62 (s, 1H), 8.93 (s, 1H), 8.22-7.76 (m, 2H), 7.57-7.48 |
| | (m, 2H), 7.43-7.35 (m, 3H), 4.11 (s, 3H), 2.54 (s, 1H), 2.17 (s, 6H). |
| 365 | N-(4-(1-cyclopropyl-3-phenyl-1H-pyrazol-4-yl)-7-methoxypyrido[3,2-d]pyrimidin- |
| | 6-y1)-1 -(trifluoromethyl)-1H-pyrazole-4-carboxamide |
| | 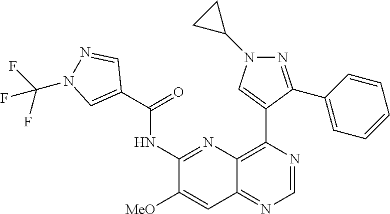 [SMILES: COc1cc2ncnc(-c3cn(C4CC4)nc3-c3ccccc3)c2nc1NC(=O)c1cnn(C(F)(F)F)c1] |
| | Halide-D; RCONH₂: 1-(trifluoromethyl)-1H-pyrazole-4-carboxamide |
| | (Preparation 76) |
| | HPLC: XBridge Shield RP18 OBD, 30 × 150 mm, 5 μm; 24-66% MeCN/H₂O (10 |
| | mM NH₄HCO₃ + 0.1% NH₄OH) |
| | White solid (5.2 mg, 13%). LCMS: m/z = 521 [M + H]⁺; ¹H NMR (400 MHz, |
| | DMSO-d₆) δ: 10.56 (s, 1H), 9.32 (d, 2H), 8.88 (s, 1H), 8.55 (s, 1H), 7.80 (s, 1H), |
| | 7.56-7.48 (m, 2H), 7.36-7.30 (m, 3H), 4.12 (s, 3H), 3.87 (m, 1H), 1.27 (m, 2H), |
| | 1.11 (m, 2H). |
| 366 | 1-(difluoromethyl)-N-(4-(3-(2-fluorophenyl)-1-methyl-1H-pyrazol-4-y1)-7- |
| | methoxypyrido[3,2-d]pyrimidin-6-yl)cyclopropane-1-carboxamide |
| | 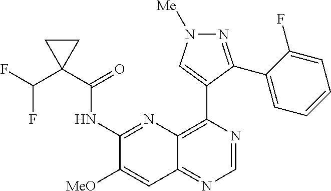 [SMILES: COc1cc2ncnc(-c3cn(C)nc3-c3ccccc3F)c2nc1NC(=O)C1(C(F)F)CC1] |
| | Halide-A; RCONH₂: 1-(difluoromethyl)cyclopropane-1-carboxamide (Preparation |
| | 69) |
| | HPLC: XBridge Shield RP18 OBD, 30 × 150 mm, 5 μm; 38-58% MeCN/H₂O (10 |
| | mM NH₄HCO₃ + 0.1% NH₄OH) |
| | White solid (31.3 mg, 21%). LCMS: m/z = 459 [M + H]⁺; ¹H NMR (300 MHz, |
| | DMSO-d₆) δ: 9.69-9.56 (m, 2H), 8.76 (s, 1H), 7.79 (s, 1H), 7.53-7.39 (m, 2H), |
| | 7.27-7.21 (m, 1H), 7.18-7.14 (m, 1H), 6.38 (t, 1H), 4.11 (s,3H), 4.06 (s, 3H), 1.30- |
| | 1.27 (m, 2H), 1.26-1.19 (m, 2H). |
| 367 | N-(4-(1-cyclopropyl-3-phenyl-1H-pyrazol-4-yl)-7-methoxypyrido[3,2-d]pyrimidin- |
| | 6-yl)-1-(trifluoromethyl)cyclopropane-1-carboxamide |
| | 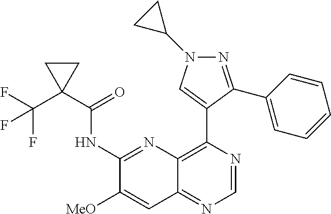 [SMILES: COc1cc2ncnc(-c3cn(C4CC4)nc3-c3ccccc3)c2nc1NC(=O)C1(C(F)(F)F)CC1] |
| | Halide-D; RCONH₂: 1-(trifluoromethyl)cyclopropane-1-carboxamide |
| | (Preparation 68) |
| | HPLC: XBridge Shield RP18 OBD, 30 × 150 mm, 5 μm; 51-71% MeCN/H₂O (10 |
| | mM NH₄HCO₃ + 0.1% NH₄OH) |
| | White solid (10.9 mg, 16%). LCMS: m/z = 495 [M + H]⁺; ¹H NMR (300 MHz, |
| | DMSO-d₆) δ: 9.72 (s, 1H), 9.29 (s, 1H), 8.86 (s, 1H), 7.77 (s, 1H), 7.52 (dd, 2H), |
| | 7.34 (dd, 3H), 4.10 (s, 3H), 3.85 (m, 1H), 1.64 (s, 2H), 1.60-1.45 (m, 2H), 1.27 (m, |
| | 2H), 1.09 (m, 2H). |
| 368 | N-(7-methoxy-4-(1-methyl-3-phenyl-1H-pyrazol-4-yl)pyrido[3,2-d]pyrimidin-6- |
| | yl)tetrahydro-1H-pyrrolizine-7a(5H)-carboxamide |
| | 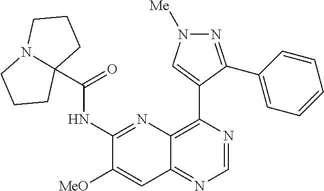 [SMILES: COc1cc2ncnc(-c3cn(C)nc3-c3ccccc3)c2nc1NC(=O)C12CCCN1CCC2] |
| | Halide-A; RCONH₂: tetrahydro-1H-pyrrolizine-7a(5H)-carboxamide (Preparation |
| | 60) |
| | HPLC: XBridge Shield RP18 OBD, 30 × 150 mm, 5 μm; 24-64% MeCN/H₂O (10 |
| | mM NH₄HCO₃ + 0.1% NH₄OH) |
| | White solid (35.1 mg, 26%). LCMS: m/z = 470 [M + H]⁺; ¹H NMR (300 MHz, |
| | DMSO-d₆) δ: 11.46 (s, 1H), 9.90 (s, 1H), 8.77 (s, 1H), 7.69 (s, 1H), 7.59-7.49 (m, |
| | 2H), 7.42-7.29 (m, 3H), 4.12 (s, 3H), 4.01 (s, 3H), 3.19 (dt, 2H), 2.82-2.68 (m, 2H), |
| | 2.29-2.14 (m, 2H), 1.97-1.81 (m, 4H), 1.72 (dd, 1H). |
| 369 | 1-(difluoromethyl)-N-(7-methoxy-4-(1-methyl-3-phenyl-1H-pyrazol-4- |
| | yl)pyrido[3,2-d]pyrimidin-6-yl)cyclopropane-1-carboxamide |
| | 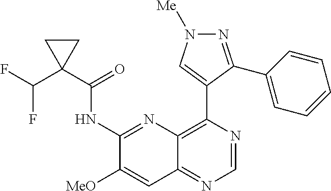 [SMILES: COc1cc2ncnc(-c3cn(C)nc3-c3ccccc3)c2nc1NC(=O)C1(C(F)F)CC1] |
| | Halide-A; RCONH₂: 1-(difluoromethyl)cyclopropane-1-carboxamide (Preparation |
| | 69) |
| | HPLC: XBridge Shield RP18 OBD, 30 × 150 mm, 5 μm; 22-62% MeCN/H₂O (10 |
| | mM NH₄HCO₃ + 0.1% NH₄OH) |
| | White solid (16.3 mg, 16%). LCMS: m/z = 451 [M + H]⁺; ¹H NMR (300 MHz, |
| | DMSO-d₆) δ: 9.66 (s, 1H), 9.36 (s, 1H), 8.84 (s, 1H), 7.76 (s, 1H), 7.56-7.47 (m, |
| | 2H), 7.33 (dd, 3H), 6.41 (t, 1H), 4.10 (s, 3H), 3.99 (s, 3H), 1.48 (d, 2H), 1.32-1.21 |
| | (m, 2H). |
| 370 | N-(4-(1-(2,2-difluoroethyl)-3-phenyl-1H-pyrazol-4-yl)-7-methoxypyrido[3,2- |
| | d]pyrimidin-6-y1)-1-(trifluoromethyl)cyclopropane-1-carboxamide |
| | 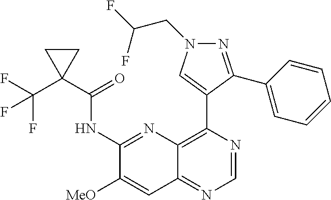 [SMILES: COc1cc2ncnc(-c3cn(CC(F)F)nc3-c3ccccc3)c2nc1NC(=O)C1(C(F)(F)F)CC1] |
| | Halide-E; RCONH₂: 1-(trifluoromethyl)cyclopropane-1-carboxamide (Preparation |
| | 68) |
| | HPLC: YMC-Actus Triart C18 ExRS, 30 × 150 mm, 5 μm; 47-67% MeCN/H₂O (10 |
| | mM NH₄HCO₃ + 0.1% NH₄OH) |
| | White solid (50.7 mg, 40%). LCMS: m/z = 519 [M + H]⁺; ¹H NMR (300 MHz, |
| | DMSO-d₆) δ: 9.63 (s, 1H), 9.09 (s, 1H), 8.81 (s, 1H), 7.67 (s, 1H), 7.38 (dq, 2H), |
| | 7.21 (p, 3H), 6.66-6.18 (m, 1H), 4.64 (td, 2H), 1.57-1.28 (m, 4H). |
| 371 | N-(4-(1-ethyl-3-phenyl-1H-pyrazol-4-yl)-7-methoxypyrido[3,2-d]pyrimidin-6-yl)- |
| | 1-(trifluoromethyl)cyclopropane-1-carboxamide |
| | 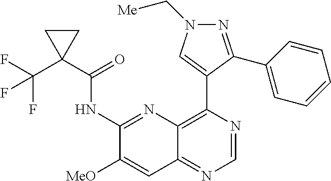 [SMILES: CCn1cc(-c2ncnc3cc(OC)c(NC(=O)C4(C(F)(F)F)CC4)nc23)c(-c2ccccc2)n1] |
| | Halide-F; RCONH₂: 1-(trifluoromethyl)cyclopropane-1-carboxamide (Preparation |
| | 68) |
| | HPLC: YMC-Actus Triart C18 ExRS, 20 × 250 mm, 5 μm; 52-82% MeCN/H₂O (10 |
| | mM NH₄HCO₃ + 0.1% NH₄OH) |
| | White solid (18.3 mg, 28%). LCMS: m/z = 467 [M + H]⁺; ¹H NMR (300 MHz, |
| | DMSO-d₆) δ: 9.75 (s, 1H), 9.27 (s, 1H), 8.88 (s, 1H), 7.78 (s, 1H), 7.52 (dd, 2H), |
| | 7.33 (dd, 3H), 4.28 (q, 2H), 4.10 (s, 3H), 1.65-1.43 (m, 7H). |

### Example 372: N-(7-methoxy-4-(3-phenyl-1H-pyrazol-4-yl)pyrido[3,2-d]pyrimidin-6-yl)-1-(trifluoromethyl)cyclopropane-1-carboxamide

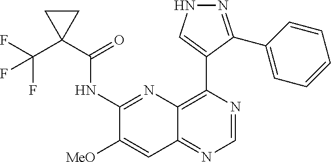 [SMILES: COc1cc2ncnc(-c3c[nH]nc3-c3ccccc3)c2nc1NC(=O)C1(C(F)(F)F)CC1]

Part 1. N-(7-methoxy-4-(3-phenyl-1-(tetrahydro-2H-pyran-2-yl)-1H-pyrazol-4-yl)pyrido[3,2-d]pyrimidin-6-yl)-1-(trifluoromethyl)cyclopropane-1-carboxamide was prepared as a yellow solid (35 mg, 68%) from 6-chloro-7-methoxy-4-(3-phenyl-1-(tetrahydro-2H-pyran-2-yl)-1H-pyrazol-4-yl)pyrido[3,2-d]pyrimidine (Preparation 234) and 1-(trifluoromethyl)cyclopropane-1-carboxamide (Preparation 68). LCMS: m/z=539 [M+H]⁺

Part 2: HCl/MeOH (4 M, 4 mL) was added to N-(7-methoxy-4-(3-phenyl-1-(tetrahydro-2H-pyran-2-yl)-1H-pyrazol-4-yl)pyrido[3,2-d]pyrimidin-6-yl)-1-(trifluoromethyl)cyclopropane-1-carboxamide (Part 1, 35 mg) in dioxane (8 mL) and the mixture stirred at rt for 1 h. The mixture was evaporated to dryness and the residue purified by prep-HPLC (YMC-Actus Triart C18 ExRS, 20×250 mm, 5 μm; 52-82% MeCN/H₂O (10 mM NH₄HCO₃+0.1% NH₄OH)) to afford the title compound as a yellow solid (6.8 mg). LCMS: m/z=455 [M+H]⁺; ¹H NMR (400 MHZ, DMSO-d₆) δ: 13.48 (s, 1H), 9.70 (s, 1H), 9.24 (s, 1H), 8.88 (s, 1H), 7.77 (s, 1H), 7.55-7.49 (m, 2H), 7.36 (s, 3H), 4.08 (s, 3H), 1.60 (s, 2H), 1.56-1.42 (m, 2H).

### Example 373 and 374: (1S,5S)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide

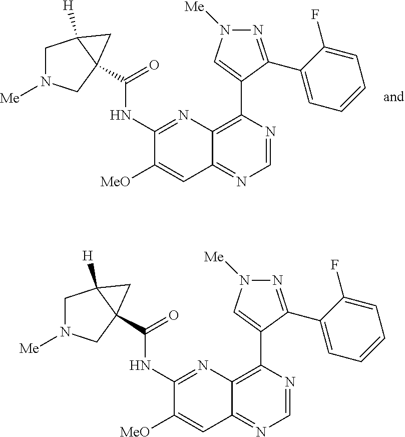 [SMILES: COc1cc2ncnc(-c3cn(C)nc3-c3ccccc3F)c2nc1NC(=O)[C@@]12C[C@@H]1CN(C)C2 ; COc1cc2ncnc(-c3cn(C)nc3-c3ccccc3F)c2nc1NC(=O)[C@]12C[C@H]1CN(C)C2]

Part 1. TFA was added to tert-butyl 1-((4-(3-(2-fluorophenyl)-1-methyl-1H-pyrazol-4-yl)-7-methoxypyrido[3,2-d]pyrimidin-6-yl)carbamoyl)-3-azabicyclo[3.1.0]hexane-3-carboxylate (Preparation 258, 100 mg, 0.178 mmol) in DCM at rt and the mixture was stirred at rt for 1 h. The reaction mixture was evaporated to dryness and the residue dissolved in DCM (10 mL). To this was added HCHO and NaBH(OAc)₃ (75.4 mg, 0.356 mmol) and the mixture stirred at rt for 2 h. The reaction mixture was extracted with EtOAc (3×50 mL) and the combined organics dried (Na₂SO₄) and evaporated to dryness in vacuo. The residue was purified by prep-HPLC (YMC-Actus Triart C18 ExRS, 30×150 mm, 5 μm; 26-53% MeCN/H₂O (10 mM NH₄HCO₃+0.1% NH₄OH)) to afford N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide as a white solid (50 mg) which was used in Part 2.

Part 2. N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide (Part 1, 50 mg) was further purified by chiral-SFC (CHIRAL ART Amylose-C NEO 30×250 mm, 5 μm; 40% MeOH (0.1% 2M NH₃—MeOH) in CO₂ to afford:

Peak 1, white solid (7.3 mg): Example 373: (1S,5S)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=474 [M+H]⁺; ¹H NMR (300 MHz, DMSO-d₆) δ: 9.52 (s, 1H), 9.24 (s, 1H), 8.74 (s, 1H), 7.68 (s, 1H), 7.54-7.48 (m, 1H), 7.44-7.38 (m, 1H), 7.27-7.21 (m, 1H), 7.17-7.10 (m, 1H), 4.07 (s, 3H), 4.00 (s, 3H), 3.25 (d, 1H), 2.94 (d, 1H), 2.73-2.68 (m, 1H), 2.45-2.38 (m, 1H), 2.30-2.25 (m, 3H), 2.14-2.08 (m, 1H), 1.41-1.38 (m, 1H), 1.34-1.30 (m, 1H).

Peak 2, white solid (12.1 mg): Example 374: (1R,5R)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(3-(2-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=474 [M+H]⁺; ¹H NMR (300 MHz, DMSO-d₆) δ: 9.52 (s, 1H), 9.24 (s, 1H), 8.74 (s, 1H), 7.68 (s, 1H), 7.54-7.48 (m, 1H), 7.46-7.38 (m, 1H), 7.27-7.21 (m, 1H), 7.17-7.10 (m, 1H), 4.07 (s, 3H), 4.00 (s, 3H), 3.25 (d, 1H), 2.94 (d, 1H), 2.73-2.68 (m, 1H), 2.43-2.38 (m, 1H), 2.30-2.26 (m, 3H), 2.14-2.08 (m, 1H), 1.41-1.38 (m, 1H), 1.34-1.30 (m, 1H).

### Example 375 and 376: (1S,5S)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide and (1R,5R)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide

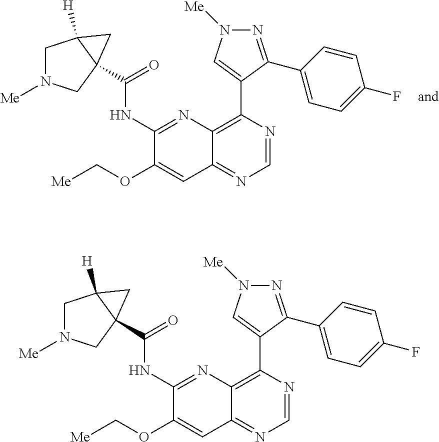 [SMILES: CCOc1cc2ncnc(-c3cn(C)nc3-c3ccc(F)cc3)c2nc1NC(=O)[C@@]12C[C@@H]1CN(C)C2 ; "and" ; CCOc1cc2ncnc(-c3cn(C)nc3-c3ccc(F)cc3)c2nc1NC(=O)[C@]12C[C@H]1CN(C)C2]

The title compounds were prepared from tert-butyl 1-((7-ethoxy-4-(3-(4-fluorophenyl)-1-methyl-1H-pyrazol-4-yl)pyrido[3,2-d]pyrimidin-6-yl)carbamoyl)-3-azabicyclo[3.1.0]hexane-3-carboxylate (Preparation 259) using an analogous 2-part procedure to that described for Example 373 and 374.

Peak 1, white solid (68.8 mg, 46%): Example 375: (1S,5S)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide or (1R,5R)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide. LCMS: m/z=488 [M+H]⁺; ¹H NMR (300 MHz, DMSO-d₆) δ: 9.35 (s, 1H), 9.25 (s, 1H), 8.84 (s, 1H), 7.69 (s, 1H), 7.62-7.50 (m, 2H), 7.26-7.09 (m, 2H), 4.34 (q, 2H), 3.98 (s, 3H), 3.23 (d, 1H), 2.95 (d, 1H), 2.68 (d, 1H), 2.39 (dd, 1H), 2.30 (s, 3H), 2.09 (q, 1H), 1.51-1.36 (m, 4H), 1.32 (dd, 1H).

Peak 2, white solid (53.4 mg, 36%): Example 376: (1R,5R)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide or (1S,5S)—N-(7-ethoxy-4-(3-(4-fluorophenyl)-1-methyl-1H-pyrazol-4-yl)pyrido[3,2-d]pyrimidin-6-yl)-3-methyl-3-azabicyclo[3.1.0]hexane-1-carboxamide. LCMS: m/z=488 [M+H]⁺; ¹H NMR (300 MHz, DMSO-d₆) δ: δ 9.35 (s, 1H), 9.26 (s, 1H), 8.84 (s, 1H), 7.69 (s, 1H), 7.56 (m, 2H), 7.26-7.10 (m, 2H), 4.34 (q, 2H), 3.98 (s, 3H), 3.24 (d, 1H), 2.95 (d, 1H), 2.69 (d, 1H), 2.42 (s, 1H), 2.30 (s, 3H), 2.10 (s, 1H), 1.52-1.37 (m, 4H), 1.32 (dd, 1H).

### Example 377 and 378: (1S,5S)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide and (1R,5R)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide

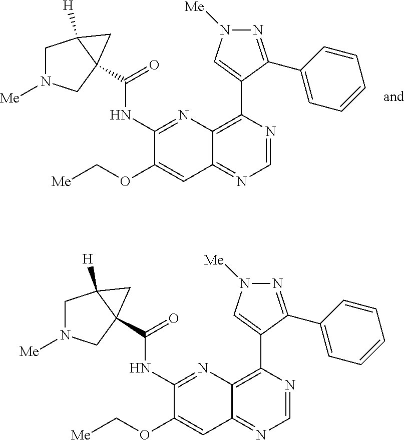 [SMILES: CCOc1cc2ncnc(-c3cn(C)nc3-c3ccccc3)c2nc1NC(=O)[C@@]12C[C@@H]1CN(C)C2 ; CCOc1cc2ncnc(-c3cn(C)nc3-c3ccccc3)c2nc1NC(=O)[C@]12C[C@H]1CN(C)C2]

Part 1. RuPhos Pd(59.3 mg, 71 μmol) and Cs₂CO₃ (345 mg, 1.06 mmol) were added to 6-chloro-7-ethoxy-4-(1-methyl-3-phenyl-1H-pyrazol-4-yl)pyrido[3,2-d]pyrimidine (Preparation 231, 260 mg, 0.710 mmol) and tert-butyl 1-carbamoyl-3-azabicyclo[3.1.0]hexane-3-carboxylate (Preparation 65, 239 mg, 1.06 mmol) in dioxane (10 mL) at rt and the resulting mixture was heated to 100° C. for 16 h under N₂. The reaction mixture was diluted with EtOAc (100 mL) and washed with water (3×100 mL), saturated brine (100 mL). The combined organics were dried (Na₂SO₄) and evaporated to dryness in vacuo. The residue was purified by prep-TLC (20:1 DCM/MeOH) to afford tert-butyl 1-((7-ethoxy-4-(1-methyl-3-phenyl-1H-pyrazol-4-yl)pyrido[3,2-d]pyrimidin-6-yl)carbamoyl)-3-azabicyclo[3.1.0]hexane-3-carboxylate as a yellow solid (260 mg, 66%). LCMS: m/z=556 [M+H]⁺.

Part 2: TFA (2 mL) was added to tert-butyl 1-((7-ethoxy-4-(1-methyl-3-phenyl-1H-pyrazol-4-yl)pyrido[3,2-d]pyrimidin-6-yl)carbamoyl)-3-azabicyclo[3.1.0]hexane-3-carboxylate (Part 1, 240 mg. 0.431 mmol) in DCM (4 mL) and the resulting mixture stirred at rt for 2 h. The reaction mixture was evaporated to dryness and the residue purified by prep-TLC (10:1 DCM/MeOH) to afford N-(7-ethoxy-4-(1-methyl-3-phenyl-1H-pyrazol-4-yl)pyrido[3,2-d]pyrimidin-6-yl)-3-azabicyclo[3.1.0]hexane-1-carboxamide as a yellow solid (160 mg, 82%). LCMS: m/z=456 [M+H]⁺.

Part 3. NaBH(OAc)₃ (174 mg, 0.822 mmol) was added to N-(7-ethoxy-4-(1-methyl-3-phenyl-1H-pyrazol-4-yl)pyrido[3,2-d]pyrimidin-6-yl)-3-azabicyclo[3.1.0]hexane-1-carboxamide (Part 2, 150 mg, 0.329 mmol) and HCHO (98.4 mg, 3.28 mmol) in DCM (10 mL). The resulting mixture stirred at rt for 2 h. The mixture was evaporated to dryness, and the residue purified by prep-HPLC (XBridge Shield RP18 OBD Column. 30×150 mm. 5 μm; 19-58% MeCN/H₂O (10 mM NH₄HCO₃+0.1% NH₄OH)) to afford N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide as a white solid (100 mg, 65%). The title compounds were obtained from N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide (Part 3, 100 mg) by chiral-HPLC (CHIRAL ART Cellulose-SC. 20×250 mm. 5 μm; 10% IPA/(3:1 Hex/DCM (0.5% 2M NH₃—MeOH).

Peak 1, white solid (44 mg, 44%): Example 377: (1S,5S)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1R,5R)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=470 [M+H]⁺; ¹H NMR (300 MHz, DMSO-d₆) δ: 9.29 (s, 2H), 8.83 (s, 1H), 7.70 (s, 1H), 7.51 (dd, 2H), 7.33 (dd, 3H), 4.35 (q, 2H), 3.99 (s, 3H), 3.3-3.26 (m, 1H), 2.95-2.61 (m, 2H), 2.42 (s, 1H), 2.31 (s, 3H), 2.11 (s, 1H), 1.52-1.33 (m, 5H).

Peak 2, white solid (41.1 mg, 41%): Example 378: (1R,5R)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1S,5S)—N-(7-ethoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=470 [M+H]⁺; ¹H NMR (300 MHz, DMSO-d₆) δ: 9.28 (d, 2H), 8.83 (s, 1H), 7.69 (s, 1H), 7.51 (dd, 2H), 7.33 (dd, 3H), 4.34 (q, 2H), 3.99 (s, 3H), 3.24 (d, 1H), 2.95 (d, 1H), 2.69 (d, 1H), 2.42 (s, 1H), 2.30 (s, 3H), 2.11 (dd, 1H), 1.46-1.32 (m, 5H).

### Example 379 and 380: (1S,5S)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide and (1R,5R)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide

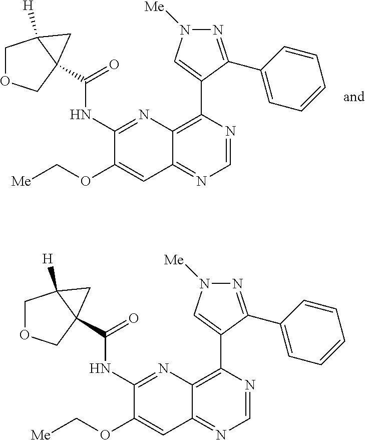 [SMILES: CCOc1cc2ncnc(-c3cn(C)nc3-c3ccccc3)c2nc1NC(=O)[C@]12COC[C@H]1C2 ; CCOc1cc2ncnc(-c3cn(C)nc3-c3ccccc3)c2nc1NC(=O)[C@@]12COC[C@@H]1C2]

Part 1. RuPhos Pd(45.6 mg, 54.6 μmol) and Cs₂CO₃ (13.3 mg, 40.9 μmol) were added to 6-chloro-7-ethoxy-4-(1-methyl-3-phenyl-1H-pyrazol-4-yl)pyrido[3,2-d]pyrimidine (Preparation 231, 200 mg, 546 μmol) and 3-oxabicyclo[3.1.0]hexane-1-carboxamide (Preparation 64, 104 mg, 818 μmol) in dioxane (10 mL) at rt and the resulting mixture heated to 80° C. for 2 h under N₂. The reaction mixture was diluted with EtOAc (100 mL), washed with water (3×100 mL) and saturated brine (100 mL). The combined organics were dried (Na₂SO₄) and evaporated to dryness and the residue purified by prep-TLC (20:1 DCM/MeOH) followed by prep-HPLC (YMC-Actus Triart C18, 30×150 mm, 5 μm; 42-52% MeCN/H₂O (10 mM NH₄HCO₃+0.1% NH₄OH)) to afford N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide as a white solid (100 mg, 40%).

Part 2. N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide (Part 1, 100 mg, 0.219 mmol) was purified by prep-HPLC to afford:

Peak 1, white solid (15.7 mg, 16%): Example 379: (1S,5S)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide or (1R,5R)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide. LCMS: m/z=457 [M+H]⁺; ¹H NMR (300 MHz, DMSO-d₆) δ: 9.48 (s, 1H), 9.19 (s, 1H), 8.85 (s, 1H), 7.71 (s, 1H), 7.55-7.45 (m, 2H), 7.36-7.28 (m, 3H), 4.34 (q, 2H), 4.10-3.96 (m, 5H), 3.79 (s, 2H), 2.32 (s, 1H), 1.59-1.40 (m, 4H), 0.98 (t, 1H).

Peak 2, white solid (20 mg, 20%): Example 380: (1R,5R)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide or (1S,5S)—N-(7-ethoxy-4-(1-methyl-3-phenyl-1H-pyrazol-4-yl)pyrido[3,2-d]pyrimidin-6-yl)-3-oxabicyclo[3.1.0]hexane-1-carboxamide. LCMS: m/z=457 [M+H]⁺; ¹H NMR (300 MHz, DMSO-d₆) δ: 9.48 (s, 1H), 9.19 (s, 1H), 8.85 (s, 1H), 7.71 (s, 1H), 7.50 (dd, 2H), 7.32 (dd, 3H), 4.34 (q, 2H), 4.10-3.96 (m, 5H), 3.79 (s, 2H), 2.32 (s, 1H), 1.54 (dd, 1H), 1.46 (t, 3H), 0.98 (t, 1H).

### Example 381 and 382: (1S,5S)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide and (1R,5R)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide

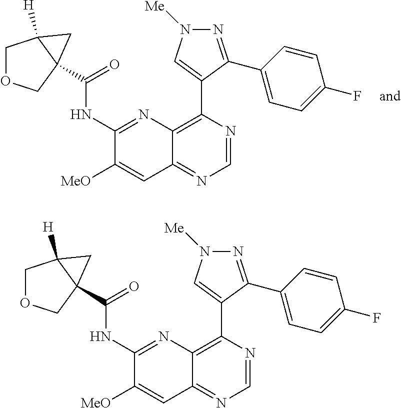 [SMILES: COc1cc2ncnc(-c3cn(C)nc3-c3ccc(F)cc3)c2nc1NC(=O)[C@]12COC[C@H]1C2 ; COc1cc2ncnc(-c3cn(C)nc3-c3ccc(F)cc3)c2nc1NC(=O)[C@@]12COC[C@@H]1C2]

Part 1. To a mixture of 3-oxabicyclo[3.1.0]hexane-1-carboxylic acid (54.8 mg, 0.428 mmol) was added TEA (216 mg, 2.14 mmol) in THF (10 mL) was added 2,4,6-trichlorobenzoyl chloride (104 mg, 0.428 mmol) at 0° C., the reaction mixture was stirred at 25° C. for 2 h. To this was added 4-(3-(4-fluorophenyl)-1-methyl-1H-pyrazol-4-yl)-7-methoxypyrido[3,2-d]pyrimidin-6-amine (Preparation 218, 150 mg, 0.428 mmol) and the mixture stirred at 60° C. overnight. The reaction mixture was evaporated to dryness and the residue diluted with EtOAc (100 mL), washed with brine (2×50 mL), dried (Na₂SO₄) and evaporated to dryness in vacuo. The residue was purified by prep-HPLC (XBridge Shield RP18 OBD Column, 30×150 mm, 5 μm; 8-56% MeCN/H₂O (10 mM NH₄HCO₃+0.1% NH₄OH)) to give N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide as a yellow solid.

Part 2. N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide (Part 1, 80 mg, 0.173 mmol) was purified by chiral-HPLC (CHIRALPAK IG, 20×250 mm, 5 μm; 5% EtOH/(Hex/DCM=3:1 (0.5% 2M NH₃—MeOH)) to afford:

Peak 1, white solid (21.5 mg, 27%): Example 381, (1S,5S)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide or (1R,5R)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide. LCMS: m/z=461 [M+H]⁺; ¹H NMR (300 MHz, DMSO-d₆) δ: 9.53 (s, 1H), 9.24 (s, 1H), 8.88 (s, 1H), 7.73 (s, 1H), 7.62-7.40 (m, 2H), 7.15 (t, 2H), 4.18-3.92 (m, 8H), 3.86-3.69 (m, 2H), 2.34 (dd, 1H), 2.08 (s, OH), 1.54 (d, 1H), 0.97 (t, 1H).

Peak 2, white solid (17.7 mg, 22%): Example 382, (1R,5R)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide or (1S,5S)—N-(4-(3-(4-fluorophenyl)-1-methyl-1H-pyrazol-4-yl)-7-methoxypyrido[3,2-d]pyrimidin-6-yl)-3-oxabicyclo[3.1.0]hexane-1-carboxamide. LCMS: m/z=461 [M+H]⁺; ¹H NMR (300 MHz, DMSO-d₆) δ: 9.52 (s, 1H), 9.24 (s, 1H), 8.88 (s, 1H), 7.72 (s, 1H), 7.64-7.42 (m, 2H), 7.16 (d, 2H), 4.12-3.96 (m, 8H), 3.79 (d, 2H), 2.42-2.27 (m, 1H), 2.08 (s, OH), 1.54 (d, 1H), 1.23 (s, OH), 0.97 (t, 1H).

### Example 383: N-(7-methoxy-4-(1-methyl-3-phenyl-1H-pyrazol-4-yl)pyrido[3,2-d]pyrimidin-6-yl)-4-(trifluoromethyl)tetrahydro-2H-pyran-4-carboxamide

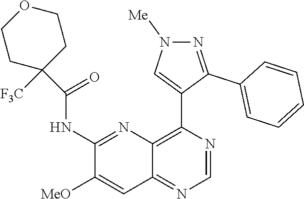 [SMILES: COc1cc2ncnc(-c3cn(C)nc3-c3ccccc3)c2nc1NC(=O)C1(C(F)(F)F)CCOCC1]

The title compound was prepared as a white solid (11 mg, 43%) from 4-(trifluoromethyl)tetrahydro-2H-pyran-4-carboxylic acid, 2,4,6-trichlorobenzoyl chloride and 6-chloro-7-methoxy-4-(1-methyl-3-phenyl-1H-pyrazol-4-yl)pyrido[3,2-d]pyrimidine (Preparation 242) using an analogous method to that described for part 1 of Example 381. Prep-HPLC (XBridge Shield RP18 OBD Column, 30×150 mm, 5 μm; 8-56% MeCN/H₂O (10 mM NH₄HCO₃+0.1% NH₄OH)). LCMS: m/z=513 [M+H]⁺; ¹H NMR (300 MHz, DMSO-d₆) δ: 10.36 (s, 1H), 8.97 (s, 1H), 8.83 (s, 1H), 7.83 (s, 1H), 7.48 (dd, 2H), 7.31 (dt, 3H), 4.05-3.94 (m, 8H), 3.48 (t, 2H), 2.53 (m, 2H), 1.86 (t, 2H).

### Example 384 and 385: (1S,5S)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide and (1R,5R)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide

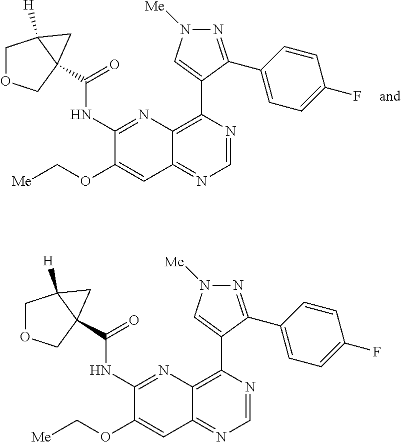 [SMILES: CCOc1cc2ncnc(-c3cn(C)nc3-c3ccc(F)cc3)c2nc1NC(=O)[C@]12COC[C@H]1C2 ; CCOc1cc2ncnc(-c3cn(C)nc3-c3ccc(F)cc3)c2nc1NC(=O)[C@@]12COC[C@@H]1C2]

Part 1. BINAP Pd(48.5 mg, 52.1 μmol) and Cs₂CO₃ (254 mg, 781 μmol) were added to 6-chloro-7-ethoxy-4-(3-(4-fluorophenyl)-1-methyl-1H-pyrazol-4-yl)pyrido[3,2-d]pyrimidine (Preparation 232, 200 mg, 521 μmol) and 3-oxabicyclo[3.1.0]hexane-1-carboxamide (Preparation 64, 99.2 mg, 781 μmol) in dioxane (10 mL) at rt and the resulting mixture heated to 100° C. for 16 h under N₂. The reaction mixture was diluted with EtOAc (100 mL), washed with water (3×100 mL) and saturated brine (100 mL). The organic layer was dried (Na₂SO₄) evaporated to dryness in vacuo. The residue was purified by prep-TLC (20:1 DCM/MeOH) followed by prep-HPLC (XBridge Prep OBD C18 Column, 30×150 mm, 5 μm; 35-55% MeCN/H₂O (10 mM NH₄HCO₃+0.1% NH₄OH to afford N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide as a white solid (80 mg, 32%).

Part 2. N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide (80 mg, 0.168 mmol) was further purified by prep-Chiral-HPLC (CHIRAL ART Amylose-SA, 20×250 mm, 5 μm; 15% EtOH/(Hex (0.5% 2M NH₃—MeOH)) to afford:

Peak 1, white solid (28.4 mg, 36%): Example 384: (1S,5S)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide or (1R,5R)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide. LCMS: m/z=475 [M+H]⁺; ¹H NMR (300 MHz, DMSO-d₆) δ: 9.46 (s, 1H), 9.25 (s, 1H), 8.86 (s, 1H), 7.70 (s, 1H), 7.55 (dd, 2H), 7.15 (t, 2H), 4.34 (q, 2H), 4.10-3.96 (m, 5H), 3.85-3.72 (m, 2H), 2.31 (S, 1H), 1.54 (dd, 1H), 1.46 (t, 3H), 0.98 (t, 1H).

Peak 2, white solid (25 mg, 31%): Example 385: (1R,5R)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide or (1S,5S)—N-(7-ethoxy-4-(3-(4-fluorophenyl)-1-methyl-1H-pyrazol-4-yl)pyrido[3,2-d]pyrimidin-6-yl)-3-oxabicyclo[3.1.0]hexane-1-carboxamide. LCMS: m/z=475 [M+H]⁺; ¹H NMR (300 MHz, DMSO-d₆) δ: 9.46 (s, 1H), 9.25 (s, 1H), 8.86 (s, 1H), 7.70 (s, 1H), 7.60-7.50 (m, 2H), 7.22-7.09 (m, 2H), 4.33 (q, 2H), 4.10-3.96 (m, 5H), 3.85-3.72 (m, 2H), 2.32 (s, 1H), 1.54 (dd, 1H), 1.46 (t, 3H), 0.98 (t, 1H).

### Example 386 and 387: (1S,5S)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide and (1R,5R)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide

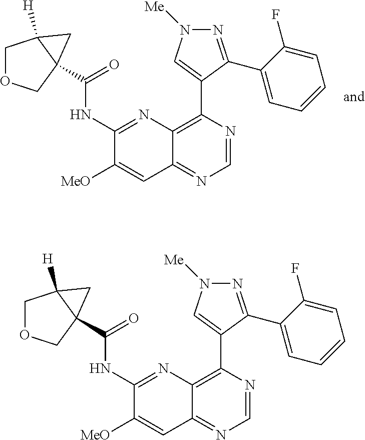 [SMILES: COc1cc2ncnc(-c3cn(C)nc3-c3ccccc3F)c2nc1NC(=O)[C@]12COC[C@H]1C2 ; COc1cc2ncnc(-c3cn(C)nc3-c3ccccc3F)c2nc1NC(=O)[C@@]12COC[C@@H]1C2]

Part 1: N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide was prepared as a white solid (30 mg) from 6-chloro-4-(3-(2-fluorophenyl)-1-methyl-1H-pyrazol-4-yl)-7-methoxypyrido[3,2-d]pyrimidine (Preparation 237) and 3-oxabicyclo[3.1.0]hexane-1-carboxamide (Preparation 64) using an analogous method to that described for Example 232, except the crude was purified by YMC-Actus Triart C18 ExRS, 30×150 mm, 5 μm; 28-51% MeCN/H₂O (10 mM NH₄HCO₃+0.1% NH₄OH)

Part 2. N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide (Part 1) was separated by chiral-HPLC (CHIRALPAK IG, 30×250 mm, 5 μm; 35% [50% MeOH/DCM (+0.1% 2M NH₃—MeOH)] in CO₂ to afford the title compounds as yellow solid.

Example 386, Peak 1 (8.8 mg): (1S,5S)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide or (1R,5R)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide. LCMS: m/z=461 [M+H]⁺; ¹H NMR (400 MHZ, DMSO-d₆) δ: 9.50 (s, 1H), 9.41 (s, 1H), 8.77 (s, 1H), 7.72 (s, 1H), 7.52 (td, 1H), 7.45-7.38 (m, 1H), 7.24 (td, 1H), 7.16-7.08 (m, 1H), 4.07-4.01 (m, 8H), 3.79 (d, 2H), 2.38-2.35 (m, 1H), 1.55 (dd, 1H), 0.98 (t, 1H).

Example 387, Peak 2 (6.8 mg): (1R,5R)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide or (1S,5S)—N-(4-(3-(2-fluorophenyl)-1-methyl-1H-pyrazol-4-yl)-7-methoxypyrido[3,2-d]pyrimidin-6-yl)-3-oxabicyclo[3.1.0]hexane-1-carboxamide. LCMS: m/z=461 [M+H]⁺; ¹H NMR (400 MHZ, DMSO-d₆) δ: 9.50 (s, 1H), 9.41 (s, 1H), 8.77 (s, 1H), 7.71 (s, 1H), 7.52 (td, 1H), 7.45-7.39 (m, 1H), 7.24 (td, 7.5 Hz, 1H), 7.16-7.09 (m, 1H), 4.07-4.01 (m, 8H), 3.79 (d, J=1.3 Hz, 2H), 2.38-2.32 (m, 1H), 1.55 (dd, 1H), 0.98 (t, 1H).

### Example 388 and 389: (1S,5S)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide and (1R,5R)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide

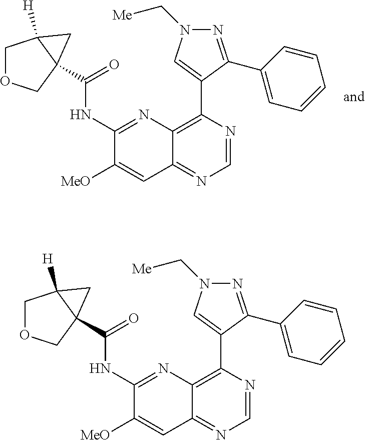 [SMILES: CCn1cc(-c2ncnc3cc(OC)c(NC(=O)[C@]45COC[C@H]4C5)nc23)c(-c2ccccc2)n1 ; CCn1cc(-c2ncnc3cc(OC)c(NC(=O)[C@@]45COC[C@@H]4C5)nc23)c(-c2ccccc2)n1]

The title compounds were prepared as white solid from 6-chloro-4-(1-ethyl-3-phenyl-1H-pyrazol-4-yl)-7-methoxypyrido[3,2-d]pyrimidine (Preparation 239) and 3-oxabicyclo[3.1.0]hexane-1-carboxamide (Preparation 64) using an analogous 2-part process as described for Examples 386 and 387.

Example 388, Peak 1(21.9 mg): (1S,5S)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide or (1R,5R)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide. LCMS: m/z=457 [M+H]⁺; ¹H NMR (400 MHZ, DMSO-d₆) δ: 9.53 (s, 1H), 9.29 (s, 1H), 8.86 (s, 1H), 7.73 (s, 1H), 7.56-7.49 (m, 2H), 7.38-7.28 (m, 3H), 4.27 (q, 2H), 4.07 (s, 5H), 3.78 (d, 2H), 2.33 (s, 1H), 1.60-1.49 (m, 4H), 0.97 (t, 1H).

Example 389, Peak 2 (11.9 mg): (1R,5R)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide or (1S,5S)—N-(4-(1-ethyl-3-phenyl-1H-pyrazol-4-yl)-7-methoxypyrido[3,2-d]pyrimidin-6-yl)-3-oxabicyclo[3.1.0]hexane-1-carboxamide. LCMS: m/z=457 [M+H]⁺; ¹H NMR (400 MHZ, DMSO-d₆) δ: 9.53 (s, 1H), 9.29 (s, 1H), 8.86 (s, 1H), 7.73 (s, 1H), 7.56-7.48 (m, 2H), 7.38-7.27 (m, 3H), 4.27 (q, 2H), 4.10-3.97 (m, 5H), 3.78 (d, 2H), 2.37-2.29 (m, 1H), 1.60-1.49 (m, 4H), 0.97 (t, 1H).

### Example 390: (S)-2-(dimethylamino)-N-(7-methoxy-4-(1-methyl-3-phenyl-1H-pyrazol-4-yl)pyrido[3,2-d]pyrimidin-6-yl) propenamide

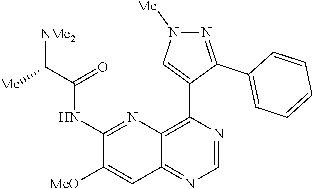 [SMILES: COc1cc2ncnc(-c3cn(C)nc3-c3ccccc3)c2nc1NC(=O)[C@H](C)N(C)C]

Part 1. A mixture of 6-chloro-7-methoxy-4-(1-methyl-3-phenyl-1H-pyrazol-4-yl)pyrido[3,2-d]pyrimidine (Preparation 242, 100 mg, 0.284 mmol), tert-butyl(S)-(1-amino-1-oxopropan-2-yl)carbamate (53 mg, 0.284 mmol), Pd₂(dba)₃CHCl₃ (29 mg, 0.028 mmol), XPhos (13.5 mg, 0.028 mmol), K₂CO₃ (78 mg, 0.568 mmol) in dioxane (5 mL) was stirred at 100° C. for 3 h under N₂. The reaction mixture was evaporated to dryness and the residue purified by column chromatography (SiO₂, 5% MeOH/DCM) to afford tert-butyl(S)-(1-((7-methoxy-4-(1-methyl-3-phenyl-1H-pyrazol-4-yl)pyrido[3,2-d]pyrimidin-6-yl)amino)-1-oxopropan-2-yl)carbamate as a white solid (80 mg, 56%). LCMS: m/z=504 [M+H]⁺.

Part 2. TFA (2 mL) was added to a solution of tert-butyl(S)-(1-((7-methoxy-4-(1-methyl-3-phenyl-1H-pyrazol-4-yl)pyrido[3,2-d]pyrimidin-6-yl)amino)-1-oxopropan-2-yl)carbamate (Part 1, 80 mg, 0.158 mmol) in DCM (5 mL) and the resulting mixture stirred at rt for 3 h. The reaction mixture was evaporated to dryness in vacuo to afford(S)-2-amino-N-(7-methoxy-4-(1-methyl-3-phenyl-1H-pyrazol-4-yl)pyrido[3,2-d]pyrimidin-6-yl) propenamide as a white solid (50 mg, 78%) which was used without further purification. LCMS: m/z=404 [M+H]⁺.

Part 3. A mixture of(S)-2-amino-N-(7-methoxy-4-(1-methyl-3-phenyl-1H-pyrazol-4-yl)pyrido[3,2-d]pyrimidin-6-yl) propenamide as a white solid (50 mg, 0.124 mmol), HCHO (1 mL) in DCM (3 mL) was stirred at rt for 1 h. To this was added NaBH(OAc)₃ (52 mg, 0.248 mmol) and stirring continued for an additional 3 h. The reaction mixture was evaporated to dryness in vacuo and the residue purified by prep-HPLC (XBridge Shield RP18 OBD, 30×150 mm, 5 μm; 23-35% MeCN/H₂O (10 mM NH₄HCO₃+0.1% NH₄OH)) to afford the title compound as a white solid (3.5 mg, 6.5%). LCMS: m/z=432 [M+H]⁺; ¹H NMR (300 MHz, DMSO-d₆) δ: 10.64 (s, 1H), 9.84 (s, 1H), 8.79 (s, 1H), 7.72 (s, 1H), 7.55 (dd, 2H), 7.36 (dd, 3H), 4.13 (s, 3H), 4.01 (s, 3H), 3.47 (t, 1H), 2.35 (s, 6H), 1.26 (d, 3H).

### Example 391: (R)—N-(4-(1-(2,2-difluoroethyl)-3-phenyl-1H-pyrazol-4-yl)-7-methoxypyrido[3,2-d]pyrimidin-6-yl)-1-methylazetidine-2-carboxamide

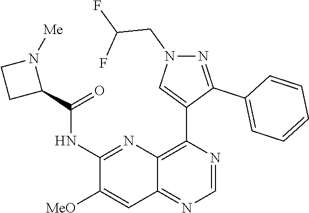 [SMILES: COc1cc2ncnc(-c3cn(CC(F)F)nc3-c3ccccc3)c2nc1NC(=O)[C@H]1CCN1C]

The title compound was prepared as a white solid (22 mg, 34%) from 6-chloro-4-(1-(2,2-difluoroethyl)-3-phenyl-1H-pyrazol-4-yl)-7-methoxypyrido[3,2-d]pyrimidine (Preparation 240) and tert-butyl (R)-2-carbamoylazetidine-1-carboxylate using an analogous 3-part procedure as described for Example 390. LCMS: m/z=480 [M+H]⁺; ¹H NMR (300 MHz, DMSO-d₆) δ: 10.56 (s, 1H), 9.82 (s, 1H), 8.70 (s, 1H), 7.62 (s, 1H), 7.43 (d t, 2H), 7.25 (d d, J=4.9, 1.9 Hz, 3H), 6.83-5.91 (m, 1H), 4.61 (td, 2H), 4.03 (s, 3H), 3.62 (t, 1H), 3.40-3.26 (m, 1H), 2.93 (q, 1H), 2.30 (s, 4H), 2.23-1.87 (m, 1H).

### Example 392 and 393: (1S,5S)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide and (1R,5R)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide

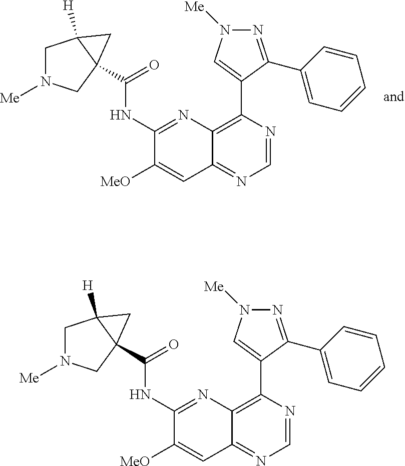 [SMILES: COc1cc2ncnc(-c3cn(C)nc3-c3ccccc3)c2nc1NC(=O)[C@@]12C[C@@H]1CN(C)C2 ; COc1cc2ncnc(-c3cn(C)nc3-c3ccccc3)c2nc1NC(=O)[C@]12C[C@H]1CN(C)C2]

Part 1. The racemic mixture of the title compounds was prepared from 6-chloro-7-methoxy-4-(1-methyl-3-phenyl-1H-pyrazol-4-yl)pyrido[3,2-d]pyrimidine (Preparation 242) and tert-butyl 1-carbamoyl-3-azabicyclo[3.1.0]hexane-3-carboxylate (Preparation 65) using an analogous method to that described for Example 390.

Part 2. The title compounds were obtained from the compound of Part 1, by chiral-HPLC (CHIRAL ART Cellulose-SC, 20×250 mm, 5 μm; 5% IPA/MtBE (0.5% 2M NH₃—MeOH)) to give:

Peak 1, (white solid, 1.2 mg), Example 392: (1S,5S)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1R,5R)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=456 [M+H]⁺; ¹H NMR (400 MHZ, MeOH-d₄) δ: 9.51 (s, 1H), 8.78 (s, 1H), 7.60 (s, 1H), 7.58-7.51 (m, 2H), 7.37 (ddd, 3H), 4.17 (s, 3H), 4.08 (s, 3H), 3.38 (d, 1H), 3.09 (d, 1H), 2.89 (d, 1H), 2.61 (dd, 1H), 2.44 (s, 3H), 2.22 (m, 1H), 1.53 (m, 1H), 1.47 (m, 1H), 1.37-1.29 (m, 4H).

Peak 2, (white solid, 1.3 mg), Example 393: (1R,5R)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1S,5S)—N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=456 [M+H]⁺; ¹H NMR (400 MHZ, DMSO-d₆) δ: 9.32 (d, 2H), 8.85 (s, 1H), 7.73 (s, 1H), 7.56-7.49 (m, 2H), 7.34 (dd, 3H), 4.08 (s, 3H), 3.99 (s, 3H), 3.25 (d, 1H), 2.95 (d, 1H), 2.70 (s, 1H), 2.44-2.37 (m, 1H), 2.31 (s, 3H), 2.12 (m, 1H), 1.41 (m, 1H), 1.32 (m, 1H), 1.24 (s, 1H).

### Example 394 and 395: (R)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide and (S)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide

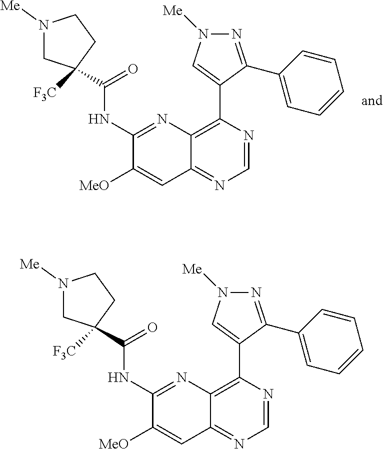 [SMILES: COc1cc2ncnc(-c3cn(C)nc3-c3ccccc3)c2nc1NC(=O)[C@@]1(C(F)(F)F)CCN(C)C1 ; COc1cc2ncnc(-c3cn(C)nc3-c3ccccc3)c2nc1NC(=O)[C@]1(C(F)(F)F)CCN(C)C1]

The title compounds were prepared from 6-chloro-7-methoxy-4-(1-methyl-3-phenyl-1H-pyrazol-4-yl)pyrido[3,2-d]pyrimidine (Preparation 242) and tert-butyl 3-carbamoyl-3-(trifluoromethyl) pyrrolidine-1-carboxylate (Preparation 298) using an analogous 2-part method as described for Example 392 and 393. Chiral-HPLC (CHIRAL ART Cellulose-SC, 20×250 mm, 5 μm; 2% EtOH/MtBE (0.1% DEA)) afforded:

Peak 1, (white solid, 6.4 mg), Example 394: (R)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide or (S)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide. LCMS: m/z=512 [M+H]⁺; ¹H NMR (400 MHZ, DMSO-d₆) δ: 11.82 (s, 1H), 9.70 (s, 1H), 8.79 (s, 1H), 7.69 (s, 1H), 7.53 (dd, 2H), 7.34 (dd, 3H), 4.11 (s, 3H), 3.98 (s, 3H), 3.50 (m, 1H), 3.22 (s, 1H), 2.59 (m, 1H), 2.54 (m, 3H), 2.40 (m, 3H).

Peak 2, (white solid, 8.2 mg), Example 395: (S)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide or (R)—N-(7-methoxy-4-(1-methyl-3-phenyl-1H-pyrazol-4-yl)pyrido[3,2-d]pyrimidin-6-yl)-1-methyl-3-(trifluoromethyl) pyrrolidine-3-carboxamide. LCMS: m/z=512 [M+H]⁺; ¹H NMR (400 MHZ, DMSO-d₆) δ: 11.82 (s, 1H), 9.70 (s, 1H), 8.79 (s, 1H), 7.69 (s, 1H), 7.58-7.49 (m, 2H), 7.40-7.30 (m, 3H), 4.11 (s, 3H), 3.98 (s, 3H), 3.50 (m, 1H), 3.22 (s, 1H), 2.59 (m, 1H), 2.54 (s, 3H), 2.48-2.34 (m, 3H).

### Example 396 and 397: (1R,5R)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide and (1S,5S)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide

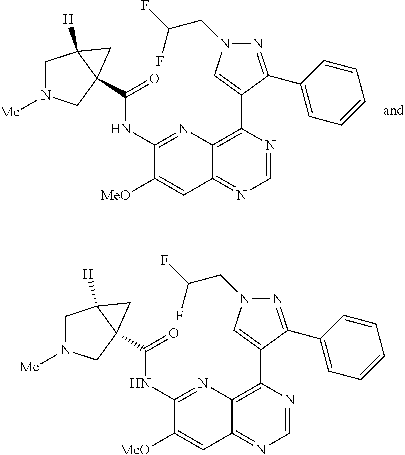 [SMILES: COc1cc2ncnc(-c3cn(CC(F)F)nc3-c3ccccc3)c2nc1NC(=O)[C@]12C[C@H]1CN(C)C2 ; COc1cc2ncnc(-c3cn(CC(F)F)nc3-c3ccccc3)c2nc1NC(=O)[C@@]12C[C@@H]1CN(C)C2]

The title compounds were prepared from 6-chloro-4-(1-(2,2-difluoroethyl)-3-phenyl-1H-pyrazol-4-yl)-7-methoxypyrido[3,2-d]pyrimidine (Preparation 240) and tert-butyl 1-carbamoyl-3-azabicyclo[3.1.0]hexane-3-carboxylate (Preparation 65) using an analogous 2-part method as described for Example 392 and 393. Chiral-HPLC (CHIRALPAK AD-H, 20×250 mm, 5 μm; 10% IPA/Hex (0.5% 2M NH₃—MeOH)) to afford:

Peak 1, (white solid, 11.2 mg), Example 396: (1R,5R)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=506 [M+H]⁺; ¹H NMR (400 MHZ, DMSO-d₆) δ: 9.34 (d, 2H), 8.90 (s, 1H), 7.75 (s, 1H), 7.57-7.47 (m, 2H), 7.35 (dt, 3H), 6.78-6.26 (m, 1H), 4.75 (td, 2H), 4.08 (s, 3H), 3.23 (d, 1H), 2.94 (d, 1H), 2.67 (d, 1H), 2.39 (dd, 1H), 2.30 (s, 3H), 2.09 (s, 1H), 1.60-1.29 (m, 2H)

Peak 2, (white solid, 10.1 mg), Example 397: (1R,5R)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(1-(2,2-difluoroethyl)-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=506 [M+H]⁺; ¹H NMR (400 MHZ, DMSO-d₆) δ: 9.34 (d, J=11.2 Hz, 2H), 8.90 (s, 1H), 7.75 (s, 1H), 7.60-7.47 (m, 2H), 7.35 (dt, J=4.5, 2.8 Hz, 3H), 6.56 (s, 1H), 4.75 (td, J=15.0, 3.8 Hz, 2H), 4.08 (s, 3H), 3.23 (d, J=8.5 Hz, 1H), 2.94 (d, J=8.9 Hz, 1H), 2.66 (s, 1H), 2.51 (p, J=1.9 Hz, 1H), 2.39-2.21 (m, 3H), 2.09 (ddd, J=8.4, 5.0, 3.4 Hz, 1H), 1.49-1.23 (m, 02H).

### Example 398 and 399: (1S,5S)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide

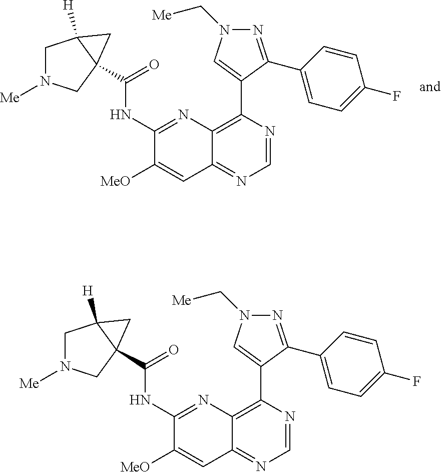 [SMILES: CCn1cc(-c2ncnc3cc(OC)c(NC(=O)[C@@]45C[C@@H]4CN(C)C5)nc23)c(-c2ccc(F)cc2)n1 ; CCn1cc(-c2ncnc3cc(OC)c(NC(=O)[C@]45C[C@H]4CN(C)C5)nc23)c(-c2ccc(F)cc2)n1]

The title compounds were prepared from 6-chloro-4-(1-ethyl-3-(4-fluorophenyl)-1H-pyrazol-4-yl)-7-methoxypyrido[3,2-d]pyrimidine (Preparation 299) and tert-butyl 1-carbamoyl-3-azabicyclo[3.1.0]hexane-3-carboxylate (Preparation 65) using an analogous 2-part method as described for Example 392 and 393. Chiral-HPLC (CHIRALPAK IC-3, 4.6×50 mm, 3 μm; 7% IPA/(3:1 Hex/DCM (+0.5% 2M NH₃—MeOH))) to afford:

Peak 1, (off-white solid, 4.8 mg), Example 398: (1S,5S)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide. LCMS: m/z=488 [M+H]⁺; ¹H NMR (400 MHZ, DMSO-d₆) δ: 9.43 (s, 1H), 9.28 (s, 1H), 8.85 (s, 1H), 7.72 (s, 1H), 7.63-7.53 (m, 2H), 7.21-7.11 (m, 2H), 4.26 (q, 2H), 4.08 (s, 3H), 3.32-3.21 (m, 1H), 2.94 (d, 1H), 2.68 (s, 1H), 2.40 (s, 1H), 2.30 (s, 3H), 2.10 (s, 1H), 1.56 (t, 3H), 1.40 (t, 1H), 1.32 (s, 1H).

Peak 1, (off-white solid, 5.4 mg), Example 399: (1R,5R)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(1-ethyl-3-(4-fluorophenyl)-1H-pyrazol-4-yl)-7-methoxypyrido[3,2-d]pyrimidin-6-yl)-3-methyl-3-azabicyclo[3.1.0]hexane-1-carboxamide. LCMS: m/z=488 [M+H]⁺; ¹H NMR (400 MHZ, DMSO-d₆) δ: 9.43 (s, 1H), 9.28 (s, 1H), 8.85 (s, 1H), 7.72 (s, 1H), 7.63-7.54 (m, 2H), 7.21-7.11 (m, 2H), 4.26 (q, 2H), 4.08 (s, 3H), 3.27 (d, 1H), 2.94 (d, 1H), 2.69 (s, 1H), 2.40 (s, 1H), 2.30 (s, 3H), 2.09 (s, 1H), 1.56 (t, 3H), 1.40 (t, 1H), 1.31 (s, 1H).

### Example 400 and 401: (1S,5S)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide and (1R,5R)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide

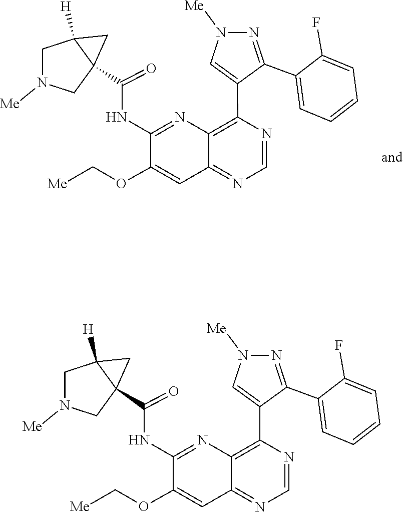 [SMILES: CCOc1cc2ncnc(-c3cn(C)nc3-c3ccccc3F)c2nc1NC(=O)[C@@]12C[C@@H]1CN(C)C2 ; CCOc1cc2ncnc(-c3cn(C)nc3-c3ccccc3F)c2nc1NC(=O)[C@]12C[C@H]1CN(C)C2]

The title compounds were prepared from 6-chloro-7-ethoxy-4-(3-(2-fluorophenyl)-1-methyl-1H-pyrazol-4-yl)pyrido[3,2-d]pyrimidine (Preparation 233) and tert-butyl 1-carbamoyl-3-azabicyclo[3.1.0]hexane-3-carboxylate (Preparation 65) using an analogous 2-part method as described for Example 392 and 393. The racemate was purified by chiral-HPLC (CHIRALPAK IG, 30×250 mm, 5 μm; 50% MeOH (+0.1% 2M NH₃—MeOH)) in CO₂ to afford:

Peak 1, (white solid, 8 mg), Example 400: (1S,5S)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide or (1R,5R)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide. LCMS: m/z=488 [M+H]⁺; ¹H NMR (400 MHZ, DMSO-d₆) δ: 9.53 (s, 1H), 9.19 (s, 1H), 8.73 (s, 1H), 7.67 (s, 1H), 7.53-7.48 (m, 1H), 7.49-7.39 (m, 1H), 7.24 (t, 1H), 7.14 (t, 1H), 4.35 (q, 2H), 4.01 (s, 3H), 3.24 (d, 1H), 2.95 (d, 1H), 2.69 (d, 1H), 2.42-2.38 (m, 1H), 2.31 (s, 3H), 2.14-2.08 (m, 1H), 1.47 (t, 3H), 1.43-1.41 (m, 1H), 1.35-1.31 (m, 1H).

Peak 2, (white solid, 10.3 mg), Example 401: (1R,5R)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide or (1S,5S)—N-(7-ethoxy-4-(3-(2-fluorophenyl)-1-methyl-1H-pyrazol-4-yl)pyrido[3,2-d]pyrimidin-6-yl)-3-methyl-3-azabicyclo[3.1.0]hexane-1-carboxamide. LCMS: m/z=488 [M+H]⁺; ¹H NMR (400 MHZ, DMSO-d₆) δ: 9.53 (s, 1H), 9.19 (s, 1H), 8.73 (s, 1H), 7.67 (s, 1H), 7.53-7.48 (m, 1H), 7.49-7.39 (m, 1H), 7.24 (t, 1H), 7.14 (t, 1H), 4.35 (q, 2H), 4.01 (s, 3H), 3.24 (d, 1H), 2.95 (d, 1H), 2.69 (d, 1H), 2.42-2.38 (m, 1H), 2.31 (s, 3H), 2.14-2.08 (m, 1H), 1.47 (t, 3H), 1.43-1.41 (m, 1H), 1.35-1.31 (m, 1H).

### Example 402:3-(dimethylamino)-N-(4-(3-(4-fluorophenyl)-1-methyl-1H-pyrazol-4-yl)-7-methoxypyrido[3,2-d]pyrimidin-6-yl)bicyclo[1.1.1]pentane-1-carboxamide

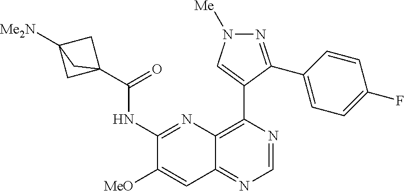 [SMILES: COc1cc2ncnc(-c3cn(C)nc3-c3ccc(F)cc3)c2nc1NC(=O)C12CC(N(C)C)(C1)C2]

Part 1. 6-Chloro-4-(3-(4-fluorophenyl)-1-methyl-1H-pyrazol-4-yl)-7-methoxypyrido[3,2-d]pyrimidine (Preparation 238, 130 mg, 0.351 mmol), tert-butyl (3-carbamoylbicyclo[1.1.1]pentan-1-yl)carbamate (Preparation 63, 79.4 mg, 0.351 mmol), K₂CO₃ (114 mg, 0.351 mmol), BINAP Pd G2 (5 mg, 5.33 μmol) in dioxane (10 mL) was stirred at 100° C. for 3 h. The reaction mixture was evaporated to dryness in vacuo and the residue purified by prep-TLC (20:1 DCM/MeOH) to afford tert-butyl (3-((4-(3-(4-fluorophenyl)-1-methyl-1H-pyrazol-4-yl)-7-methoxypyrido[3,2-d]pyrimidin-6-yl)carbamoyl)bicyclo[1.1.1]pentan-1-yl)carbamate as a yellow solid (80 mg, 41%). LCMS: m/z=560 [M+H]⁺.

Part 2. A mixture of tert-butyl (3-((4-(3-(4-fluorophenyl)-1-methyl-1H-pyrazol-4-yl)-7-methoxypyrido[3,2-d]pyrimidin-6-yl)carbamoyl)bicyclo[1.1.1]pentan-1-yl)carbamate (Part 1, 80 mg, 0.143 mmol) in DCM (10 mL) and TFA (3 mL) was stirred at rt for 1 h. The reaction mixture was evaporated to dryness and diluted with sat. Na₂CO₃ and extracted with DCM. The organics were dried (Na₂SO₄) and evaporated to dryness. The residue was purified by prep-TLC (20:1 DCM/MeOH) to afford 3-amino-N-(4-(3-(4-fluorophenyl)-1-methyl-1H-pyrazol-4-yl)-7-methoxypyrido[3,2-d]pyrimidin-6-yl)bicyclo[1.1.1]pentane-1-carboxamide as a yellow solid (50 mg, 83%). LCMS: m/z=460 [M+H]⁺.

Part 3. 3-Amino-N-(4-(3-(4-fluorophenyl)-1-methyl-1H-pyrazol-4-yl)-7-methoxypyrido[3,2-d]pyrimidin-6-yl)bicyclo[1.1.1]pentane-1-carboxamide (Part 2, 40 mg, 0.087 mmol), formaldehyde (35% aq, 0.2 mL) in MeOH (10 mL) was added NaBH₄ (9.87 mg, 0.261 mmol) and the mixture stirred at rt for 1 h. The reaction was quenched with water and evaporated to dryness and the residue purified by prep-HPLC (Xselect CSH C18 OBD, 30×150 mm 5 μm, 5-35% MeCN/H₂O (0.1% HCO₂H)) to afford the title compound as a yellow solid (3.8 mg). LCMS: m/z=488 [M+H]⁺; ¹H NMR (400 MHZ, DMSO-d₆) δ: 9.50 (d, 2H), 8.84 (s, 1H), 7.72 (s, 1H), 7.61-7.53 (m, 2H), 7.16 (t, 2H), 4.04 (d, 6H), 2.12 (d, 12H).

### Example 403: 3-(dimethylamino)-N-(4-(3-(2-fluorophenyl)-1-methyl-1H-pyrazol-4-yl)-7-methoxypyrido[3,2-d]pyrimidin-6-yl)bicyclo[1.1.1]pentane-1-carboxamide

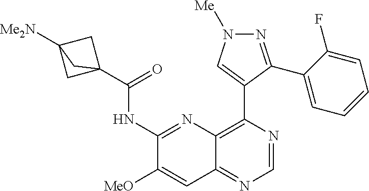 [SMILES: COc1cc2ncnc(-c3cn(C)nc3-c3ccccc3F)c2nc1NC(=O)C12CC(N(C)C)(C1)C2]

Part 1. 6-Chloro-4-(3-(2-fluorophenyl)-1-methyl-1H-pyrazol-4-yl)-7-methoxypyrido[3,2-d]pyrimidine (Preparation 237, 150 mg, 0.405 mmol), tert-butyl (3-carbamoylbicyclo[1.1.1]pentan-1-yl)carbamate (Preparation 63, 137 mg, 0.607 mmol), Cs₂CO₃ (197 mg, 0.607 mmol), BINAP Pd G2 (37 mg, 40.5 μmol) in dioxane (10 mL) was stirred at 100° C. for 3 h. The reaction mixture was extracted with EtOAc (3×50 mL). The combined organics were dried (Na₂SO₄) and evaporated to dryness in vacuo and the residue purified by prep-TLC (25:1 DCM/MeOH) to afford tert-butyl (3-((4-(3-(2-fluorophenyl)-1-methyl-1H-pyrazol-4-yl)-7-methoxypyrido[3,2-d]pyrimidin-6-yl)carbamoyl)bicyclo[1.1.1]pentan-1-yl)carbamate as a yellow solid (120 mg, 53%). LCMS: m/z=560 [M+H]⁺.

Part 2. A mixture of tert-butyl (3-((4-(3-(2-fluorophenyl)-1-methyl-1H-pyrazol-4-yl)-7-methoxypyrido[3,2-d]pyrimidin-6-yl)carbamoyl)bicyclo[1.1.1]pentan-1-yl)carbamate (Part 1, 120 mg, 0.214 mmol) in DCM (10 mL) and TFA (3 mL) was stirred at rt for 1 h. The reaction mixture was evaporated to dryness to afford 3-amino-N-(4-(3-(2-fluorophenyl)-1-methyl-1H-pyrazol-4-yl)-7-methoxypyrido[3,2-d]pyrimidin-6-yl)bicyclo[1.1.1]pentane-1-carboxamide as a yellow solid (100 mg, which was dissolved in MeOH (10 mL). To this was added NaCNBH₃ (53.9 mg, 214 μmol), HCHO (0.2 mL) and AcOH (0.5 mL) and the mixture was stirred at rt for 2 h. The resulting solution was extracted with EtOAc (3×50 mL) and the combined organics dried (Na₂SO₄) and evaporated to dryness. The residue was purified by prep-HPLC (XBridge Shield RP18 OBD, 30×150 mm, 5 μm; 11-54% MeCN/H₂O (10 mmol/L NH₄HCO₃+0.1% NH₃·H₂O)) to afford the title compound as a white solid (10.9 mg, 10%). LCMS: m/z=488 [M+H]⁺; ¹H NMR (400 MHz, DMSO-d₆) δ: 9.64 (s, 1H), 9.48 (s, 1H), 8.74 (s, 1H), 7.70 (s, 1H), 7.51 (td, 1H), 7.43 (m, 1H), 7.24 (t, 1H), 7.15 (t, 1H), 4.09 (s, 3H), 4.02 (s, 3H), 2.16 (s, 6H), 2.10 (s, 3H).

### Example 404:3-(dimethylamino)-N-(7-methoxy-4-(1-methyl-3-phenyl-1H-pyrazol-4-yl)pyrido[3,2-d]pyrimidin-6-yl)bicyclo[1.1.1]pentane-1-carboxamide

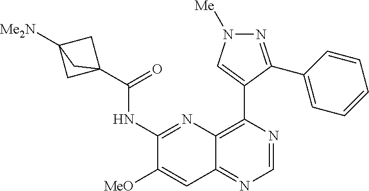 [SMILES: COc1cc2ncnc(-c3cn(C)nc3-c3ccccc3)c2nc1NC(=O)C12CC(N(C)C)(C1)C2]

Part 1. A mixture of 6-chloro-7-methoxy-4-(1-methyl-3-phenyl-1H-pyrazol-4-yl)pyrido[3,2-d]pyrimidine (Preparation 242, 115 mg, 0.326 mmol), tert-butyl (3-carbamoylbicyclo[1.1.1]pentan-1-yl)carbamate (Preparation 63, 110 mg, 0.489 mmol), K₂CO₃ (159 mg, 0.489 mmol), BINAP Pd G2 (25 mg, 0.027 mmol) in dioxane (10 mL) was stirred at 100° C. for 3 h. The reaction mixture was evaporated to dryness and the residue purified by prep-TLC 20:1 DCM/MeOH) to afford tert-butyl (3-((7-methoxy-4-(1-methyl-3-phenyl-1H-pyrazol-4-yl)pyrido[3,2-d]pyrimidin-6-yl)carbamoyl)bicyclo[1.1.1]pentan-1-yl)carbamate as a yellow solid (100 mg, 56%). LCMS: m/z=542 [M+H]⁺.

Part 2. A mixture of tert-butyl (3-((7-methoxy-4-(1-methyl-3-phenyl-1H-pyrazol-4-yl)pyrido[3,2-d]pyrimidin-6-yl)carbamoyl)bicyclo[1.1.1]pentan-1-yl)carbamate (Part 1, 110 mg, 0.125 mmol) in DCM (9 mL) and TFA (3 mL) was stirred at rt for 1 h. The reaction mixture was evaporated to dryness and the residue diluted with sat. Na₂CO₃ and extracted with DCM. The organic layer was dried (Na₂SO₄) and evaporated to dryness in vacuo. The residue was purified by prep-TLC (20:1 DCM/MeOH) to afford 3-amino-N-(7-methoxy-4-(1-methyl-3-phenyl-1H-pyrazol-4-yl)pyrido[3,2-d]pyrimidin-6-yl)bicyclo[1.1.1]pentane-1-carboxamide as a yellow solid (50 mg, 55%). LCMS: m/z=442 [M+H]⁺.

Part 3. NaBH₃CN (12.7 mg, 0.203 mmol) was added to a mixture of 3-amino-N-(7-methoxy-4-(1-methyl-3-phenyl-1H-pyrazol-4-yl)pyrido[3,2-d]pyrimidin-6-yl)bicyclo[1.1.1]pentane-1-carboxamide (Part 2, 30 mg, 67.9 μmol), formaldehyde (35% aq, 0.3 mL) and AcOH (12.7 mg, 0.203 mmol) in MeOH (10 mL) and the mixture stirred at rt for 1 h. The reaction mixture was evaporated to dryness and the residue purified by prep-HPLC (XSelect CSH C18 OBD, 30×150 mm, 5 μm; 5-30% MeCN/H₂O (0.1% HCO₂H)) to afford the title compound as a white solid (1.5 mg, 5%). LCMS: m/z=470 [M+H]⁺; ¹H NMR (400 MHZ, CDCl₃) δ: 9.61 (s, 1H), 8.82 (s, 1H), 8.46 (s, 1H), 7.60-7.54 (m, 2H), 7.43 (s, 1H), 7.32-7.26 (m, 3H), 4.05 (s, 6H), 2.33 (d, 12H).

### Example 405:3-(dimethylamino)-N-(4-(1-ethyl-3-(4-fluorophenyl)-1H-pyrazol-4-yl)-7-methoxypyrido[3,2-d]pyrimidin-6-yl)bicyclo[1.1.1]pentane-1-carboxamide

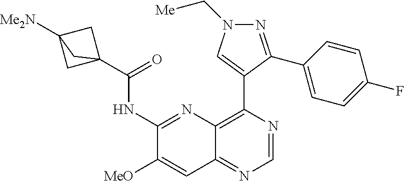 [SMILES: CCn1cc(-c2ncnc3cc(OC)c(NC(=O)C45CC(N(C)C)(C4)C5)nc23)c(-c2ccc(F)cc2)n1]

The title compound was prepared as a white solid (9.8 mg) from 6-chloro-4-(1-ethyl-3-(4-fluorophenyl)-1H-pyrazol-4-yl)-7-methoxypyrido[3,2-d]pyrimidine (Preparation 299) and tert-butyl (3-carbamoylbicyclo[1.1.1]pentan-1-yl)carbamate (Preparation 63) using an analogous 3-part method as described for Example 390. prep-HPLC (XBridge Shield RP18 OBD, 30×150 mm, 5 μm; 17-53% MeCN/H₂O (10 mM NH₄HCO₃+0.1% NH₄OH)). LCMS: m/z=503 [M+H]⁺; ¹H NMR (400 MHz, DMSO-d₆) δ: 9.55 (d, 2H), 8.84 (s, 1H), 7.72 (s, 1H), 7.63-7.54 (m, 2H), 7.22-7.11 (m, 2H), 4.27 (q, 2H), 4.09 (s, 3H), 2.15 (s, 6H), 2.08 (s, 6H), 1.56 (t, 3H).

### Example 406 and 407: (1S,5S)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide

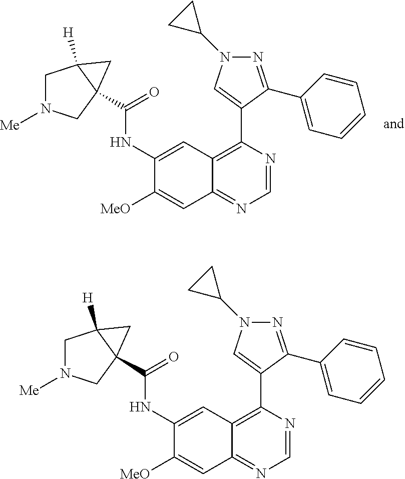 [SMILES: COc1cc2ncnc(-c3cn(C4CC4)nc3-c3ccccc3)c2cc1NC(=O)[C@@]12C[C@@H]1CN(C)C2 ; COc1cc2ncnc(-c3cn(C4CC4)nc3-c3ccccc3)c2cc1NC(=O)[C@]12C[C@H]1CN(C)C2]

Part 1. A mixture of 6-chloro-4-(1-cyclopropyl-3-phenyl-1H-pyrazol-4-yl)-7-methoxypyrido[3,2-d]pyrimidine (Preparation 245, 20 mg, 0.053 mmol), tert-butyl 1-carbamoyl-3-azabicyclo[3.1.0]hexane-3-carboxylate (Preparation 65, 23.7 mg, 0.105 mmol), BINAP Pd G2 (10 mg, 0.016 mmol), Cs₂CO₃ (34.2 mg, 0.105 mmol) in dioxane (6 mL) was stirred at 80° C. for 2 h. The reaction mixture was evaporated to dryness and residue purified by prep-TLC (35:1 DCM/MeOH) to afford tert-butyl 1-((4-(1-cyclopropyl-3-phenyl-1H-pyrazol-4-yl)-7-methoxypyrido[3,2-d]pyrimidin-6-yl)carbamoyl)-3-azabicyclo[3.1.0]hexane-3-carboxylate as a yellow solid (10 mg, 33%). LCMS: m/z=568 [M+H]⁺.

Part 2. TFA (2 mL) was added to a solution of tert-butyl 1-((4-(1-cyclopropyl-3-phenyl-1H-pyrazol-4-yl)-7-methoxypyrido[3,2-d]pyrimidin-6-yl)carbamoyl)-3-azabicyclo[3.1.0]hexane-3-carboxylate (Part 1, 40 mg, 0.070 mmol) in DCM (10 mL) and the solution stirred at rt for 2 h. The reaction mixture was evaporated to dryness in vacuo to afford N-(4-(1-cyclopropyl-3-phenyl-1H-pyrazol-4-yl)-7-methoxypyrido[3,2-d]pyrimidin-6-yl)-3-azabicyclo[3.1.0]hexane-1-carboxamide as a yellow solid (70 mg, 85%). LCMS: m/z=468 [M+H]⁺.

Part 3. NaBH(OAc)₃ (25.2 mg, 0.119 mmol) was added to solution of N-(4-(1-cyclopropyl-3-phenyl-1H-pyrazol-4-yl)-7-methoxypyrido[3,2-d]pyrimidin-6-yl)-3-azabicyclo[3.1.0]hexane-1-carboxamide (Part 2, 70 mg, 0.060 mmol), HCHO (35% aq, 2 mL) in DCM (5 mL) and the mixture stirred at rt for 2 h. The reaction was quenched with water and evaporated to dryness in vacuo. The residue was purified by prep-SFC (Chiral ND2, 3×100, 3 μm: MeOH (0.1% DEA) in CO₂) to afford the title compounds.

Peak 1, (white solid, 3.2 mg), Example 406: (1S,5S)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=482 [M+H]⁺; ¹H NMR (300 MHz, CDCl₃) δ: 9.61 (s, 1H), 8.88 (s, 1H), 8.39 (s, 1H), 7.63 (dd, 2H), 7.48 (s, 1H), 7.35 (dd, 3H), 4.12 (s, 3H), 3.81 (tt, 1H), 2.36 (d, 8H), 1.48 (s, 2H), 1.26 (s, 2H), 1.17-1.04 (m, 2H).

Peak 2, (white solid, 5.9 mg), Example 407: (1R,5R)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(1-cyclopropyl-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=482 [M+H]⁺; ¹H NMR (300 MHz, CDCl₃) δ: 9.63 (s, 1H), 8.87 (s, 1H), 8.41 (s, 1H), 7.63 (dd, 2H), 7.48 (s, 1H), 7.35 (dd, 3H), 4.12 (s, 3H), 3.80 (tt, 1H), 3.50-2.72 (m, 3H), 2.48 (s, 4H), 2.21 (s, 1H), 1.49 (dd, 3H), 1.26 (s, 1H), 1.16-1.03 (m, 2H).

### Example 408 and 409: (1S,5S)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide

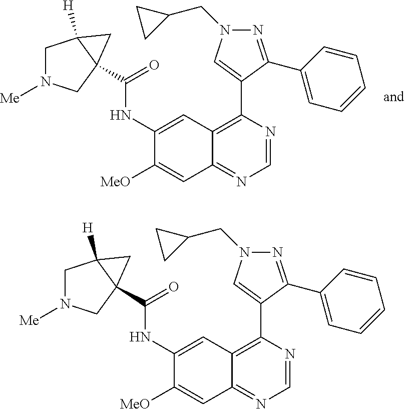 [SMILES: COc1cc2ncnc(-c3cn(CC4CC4)nc3-c3ccccc3)c2cc1NC(=O)[C@@]12C[C@@H]1CN(C)C2 ; COc1cc2ncnc(-c3cn(CC4CC4)nc3-c3ccccc3)c2cc1NC(=O)[C@]12C[C@H]1CN(C)C2]

The title compounds were prepared as white solids from 6-chloro-4-(1-(cyclopropylmethyl)-3-phenyl-1H-pyrazol-4-yl)-7-methoxypyrido[3,2-d]pyrimidine (Preparation 246) and tert-butyl 1-carbamoyl-3-azabicyclo[3.1.0]hexane-3-carboxylate (Preparation 65) using an analogous 3-part procedure to that described for Example 406 and 407. Purification by chiral-SFC (Chiral ART Amylose-C NEO, 30×250 mm, 5 μm; 35% MeOH (0.1% 2M NH₃/MeOH) in CO₂) to afford the title compounds.

Peak 1, (white solid, 3.5 mg), Example 409: (1S,5S)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=496 [M+H]⁺; ¹H NMR (300 MHz, CDCl₃) δ: 9.72 (s, 1H), 8.88 (s, 1H), 8.40 (s, 1H), 7.64 (m, 2H), 7.48 (s, 1H), 7.35 (m, 3H), 4.20-4.09 (m, 5H), 3.33 (s, 1H), 3.09 (s, 1H), 2.77 (s, 1H), 2.30 (m, 5H), 1.49 (s, 2H), 1.26 (s, 1H), 0.75-0.63 (m, 2H), 0.55 (m, 2H).

Peak 2, (white solid, 6.1 mg), Example 408: (1R,5R)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(1-(cyclopropylmethyl)-3-phenyl-1H-pyrazol-4-yl)-7-methoxypyrido[3,2-d]pyrimidin-6-yl)-3-methyl-3-azabicyclo[3.1.0]hexane-1-carboxamide. LCMS: m/z=496 [M+H]⁺; ¹H NMR (300 MHz, CDCl₃) δ: 9.71 (s, 1H), 8.88 (s, 1H), 8.40 (s, 1H), 7.64 (m, 2H), 7.48 (s, 1H), 7.35 (m, 3H), 4.20-4.09 (m, 5H), 3.39 (s, 1H), 3.12 (s, 1H), 2.82 (s, 1H), 2.47 (s, 4H), 2.16 (s, 1H), 1.49 (s, 2H), 1.26 (s, 1H), 0.75-0.59 (m, 2H), 0.59-0.50 (m, 2H).

### Example 410 and 411: (1S,5S)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide

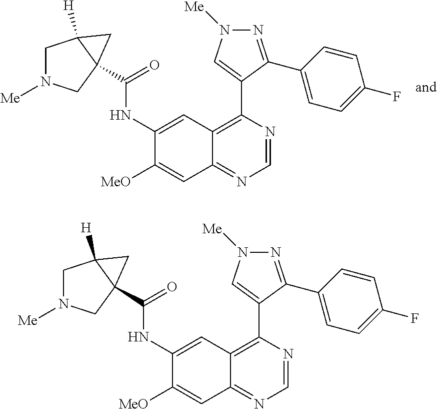 [SMILES: COc1cc2ncnc(-c3cn(C)nc3-c3ccc(F)cc3)c2cc1NC(=O)[C@@]12C[C@@H]1CN(C)C2 ; COc1cc2ncnc(-c3cn(C)nc3-c3ccc(F)cc3)c2cc1NC(=O)[C@]12C[C@H]1CN(C)C2]

The title compounds were prepared as white solids from 6-chloro-4-(3-(4-fluorophenyl)-1-methyl-1H-pyrazol-4-yl)-7-methoxypyrido[3,2-d]pyrimidine (Preparation 238) and tert-butyl 1-carbamoyl-3-azabicyclo[3.1.0]hexane-3-carboxylate (Preparation 65) using an analogous 3-part procedure to that described for Example 406 and 407. Purification by chiral-HPLC (Chiral ART Cellulose-SC, 20×250 mm, 5 μm; 10% IPA/(3:1 Hex/DCM (0.5% 2M NH₃—MeOH)) to afford the title compounds.

Peak 1, (pale-yellow solid, 7.3 mg), Example 410: (1S,5S)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=474 [M+H]⁺; ¹H NMR (400 MHZ, DMSO-d₆) δ: 9.35 (d, 2H), 8.85 (s, 1H), 7.72 (s, 1H), 7.57 (d, 2H), 7.15 (t, 2H), 4.03 (d, 6H), 3.24 (d, 1H), 2.94 (d, 1H), 2.68 (d, 1H), 2.40 (dd, 1H), 2.30 (s, 3H), 2.11 (m, 1H), 1.43-1.26 (m, 2H), 1.23 (s, 1H).

Peak 2, (white solid, 4.5 mg), Example 411: (1R,5R)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=474 [M+H]⁺; ¹H NMR (300 MHz, 400 MHZ, DMSO-d₆) δ: 9.35 (s, 1H), 9.28 (s, 1H), 8.85 (s, 1H), 7.71 (s, 1H), 7.57 (d, 2H), 7.15 (t, 2H), 4.07 (s, 3H), 3.98 (s, 3H), 3.24 (d, 1H), 2.94 (d, 1H), 2.68 (d, 1H), 2.40 (dd, 1H), 2.30 (s, 3H), 2.10 (m, 1H), 1.43-1.26 (m, 2H).

### Example 412 and 413: (1S,5S)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide

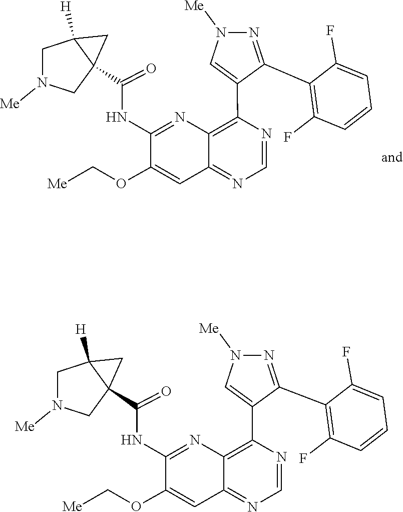 [SMILES: CCOc1cc2ncnc(-c3cn(C)nc3-c3c(F)cccc3F)c2nc1NC(=O)[C@@]12C[C@@H]1CN(C)C2 ; CCOc1cc2ncnc(-c3cn(C)nc3-c3c(F)cccc3F)c2nc1NC(=O)[C@]12C[C@H]1CN(C)C2]

The title compounds were prepared as white solids from 6-chloro-4-(3-(2,6-difluorophenyl)-1-methyl-1H-pyrazol-4-yl)-7-methoxypyrido[3,2-d]pyrimidine (Preparation 243) and tert-butyl 1-carbamoyl-3-azabicyclo[3.1.0]hexane-3-carboxylate (Preparation 65) using an analogous 3-part procedure to that described for Example 406 and 407. The racemate was purified by chiral-SFC (Chiralpak IE, 20×250 mm, 5 μm; 40% IPA/Hex (0.5% 2M NH₃—MeOH)) to afford the title compounds.

Peak 1, (pale-yellow solid, 5.9 mg), Example 412: (1S,5S)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=492 [M+H]⁺; ¹H NMR (400 MHZ, MeOH-d₄) δ: 9.97 (s, 1H), 8.63 (s, 1H), 7.52 (s, 1H), 7.45 (tt, 1H), 7.09-6.94 (m, 2H), 4.11 (d, 6H), 3.39 (d, 1H), 3.09 (d, 1H), 2.90 (d, 1H), 2.62 (dd, 1H), 2.44 (s, 3H), 2.23 (ddd, 1H), 1.57-1.43 (m, 2H).

Peak 2, (pale-yellow solid, 6 mg), Example 413: (1R,5R)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(3-(2,6-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=492 [M+H]⁺; ¹H NMR (400 MHZ, MeOH-d₄) δ: 9.97 (s, 1H), 8.63 (s, 1H), 7.52 (s, 1H), 7.45 (tt, 1H), 7.09-6.95 (m, 2H), 4.11 (d, 6H), 3.38 (d, 1H), 3.22 (s, 1H), 3.09 (d, 1H), 2.89 (d, 1H), 2.60 (dd, 1H), 2.43 (s, 3H), 2.28-2.16 (m, 1H), 1.57-1.42 (m, 2H).

### Example 414: N-(4-(1-(2-hydroxyethyl)-3-phenyl-1H-pyrazol-4-yl)-7-methoxypyrido[3,2-d]pyrimidin-6-yl)bicyclo[1.1.1]pentane-1-carboxamide

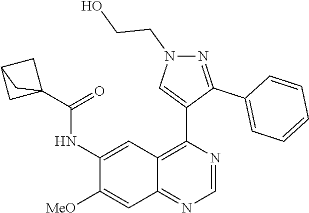 [SMILES: COc1cc2ncnc(-c3cn(CCO)nc3-c3ccccc3)c2cc1NC(=O)C12CC(C1)C2]

HCl/dioxane (4M, 5 mL) was added to a solution of N-(4-(1-(2-((tert-butyldimethylsilyl)oxy)ethyl)-3-phenyl-1H-pyrazol-4-yl)-7-methoxypyrido[3,2-d]pyrimidin-6-yl)bicyclo[1.1.1]pentane-1-carbox amide (Preparation 261, 80 mg, 0.140 mmol) in dioxane (5 mL) and the mixture stirred at rt for 1 h. The reaction mixture was evaporated to dryness in vacuo and the residue purified by prep-HPLC (YMC-Actus Triart C18, 30×150 mm, 5 μm; 38-48% MeCN/H₂O (10 mM NH₄HCO₃+0.1% NH₄OH)) to afford the title compound as a white solid (18 mg, 28%). LCMS: m/z=457 [M+H]⁺; ¹H NMR (300 MHz, DMSO-d₆) δ: 9.58 (s, 1H), 9.41 (s, 1H), 8.84 (s, 1H), 7.74 (s, 1H), 7.58-7.49 (m, 2H), 7.34 (d, 3H), 5.00 (t, 1H), 4.28 (t, 2H), 4.09 (s, 3H), 3.99-3.89 (m, 2H), 2.18 (s, 6H).

### Example 415: N-(4-(1-(2-hydroxyethyl)-3-phenyl-1H-pyrazol-4-yl)-7-methoxypyrido[3,2-d]pyrimidin-6-yl)-1-(trifluoromethyl)-1H-pyrazole-4-carboxamide

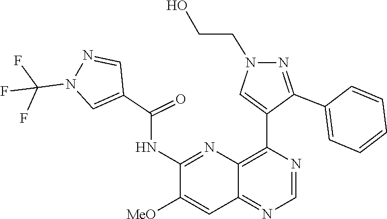 [SMILES: COc1cc2ncnc(-c3cn(CCO)nc3-c3ccccc3)c2nc1NC(=O)c1cnn(C(F)(F)F)c1]

The title compound was prepared as a white solid (13.9 mg) from N-(4-(1-(2-((tert-butyl dimethylsilyl)oxy)ethyl)-3-phenyl-1H-pyrazol-4-yl)-7-methoxypyrido[3,2-d]pyrimidin-6-yl)-1-(trifluoromethyl)-1H-pyrazole-4-carboxamide (Preparation 262) using an analogous method to that described for Example 414. The crude product was purified by prep-HPLC (YMC-Actus Triart C18, 30×150 mm, 5 μm; 26-56% MeCN/H₂O (10 mM NH₄HCO₃+0.1% NH₄OH)). LCMS: m/z=525 [M+H]⁺; ¹H NMR (400 MHZ, DMSO-d₆) δ: 10.53 (s, 1H), 9.26 (m, 2H), 8.90 (s, 1H), 8.54 (s, 1H), 7.80 (s, 1H), 7.53 (m, 2H), 7.32 (m, 3H), 4.99 (t, 1H), 4.29 (t, 2H), 4.10 (s, 3H), 3.92 (q, 2H).

### Example 416 and 417: (1R,5S)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide and (1S,5R)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide

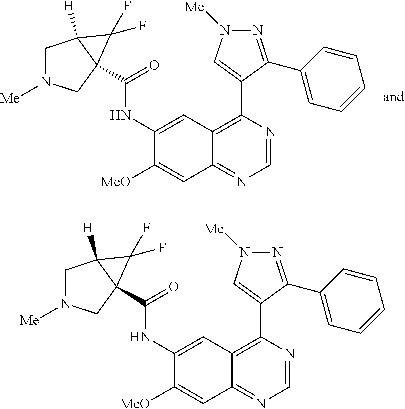 [SMILES: COc1cc2ncnc(-c3cn(C)nc3-c3ccccc3)c2cc1NC(=O)[C@]12CN(C)C[C@H]1C2(F)F ; COc1cc2ncnc(-c3cn(C)nc3-c3ccccc3)c2cc1NC(=O)[C@@]12CN(C)C[C@@H]1C2(F)F]

Part 1. To a solution of tert-butyl 6,6-difluoro-1-((7-methoxy-4-(1-methyl-3-phenyl-1H-pyrazol-4-yl)pyrido[3,2-d]pyrimidin-6-yl)carbamoyl)-3-azabicyclo[3.1.0]hexane-3-carboxylate (Preparation 260, 70 mg, 0.121 mmol) in dioxane (5 mL) was added HCl/dioxane (4M, 5 mL) and the mixture stirred at rt for 1 h. The reaction mixture was evaporated to dryness in vacuo and the residue was purified by prep-TLC (20:1 MeOH/DCM) to afford 6,6-difluoro-N-(7-methoxy-4-(1-methyl-3-phenyl-1H-pyrazol-4-yl)pyrido[3,2-d]pyrimidin-6-yl)-3-azabicyclo[3.1.0]hexane-1-carboxamide as a yellow solid (50 mg, 86%). LCMS: m/z=478 [M+H]⁺.

Part 2. To a mixture of 6,6-difluoro-N-(7-methoxy-4-(1-methyl-3-phenyl-1H-pyrazol-4-yl)pyrido[3,2-d]pyrimidin-6-yl)-3-azabicyclo[3.1.0]hexane-1-carboxamide (Part 1, 50 mg, 0.105 mmol), HCHO (35% aq, 1 mL) in DCM (10 mL) was added NaBH(OAc)₃ (44.4 mg, 0.210 mmol) and stirred at rt for 1 h. The reaction mixture was quenched with water and evaporated to dryness in vacuo. The residue was purified by prep-TLC (25:1 DCM/MeOH) to afford 6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. Purification by chiral-SFC (Chiralpak IH, 20×250 mm, 5 μm; 30% EtOH/Hex (0.5% 2M NH₃—MeOH)) to afford the title compounds.

Peak 1, (off-white solid, 3.1 mg), Example 416: (1R,5S)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1S,5R)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=492 [M+H]⁺; ¹H NMR (300 MHz, DMSO-d₆) δ: 10.53 (s, 1H), 9.19 (s, 1H), 8.87 (s, 1H), 7.76 (s, 1H), 7.50 (dd, 2H), 7.32 (dd, 3H), 4.04 (d, 6H), 2.99 (s, 3H), 2.51 (s, 3H), 2.29 (s, 2H).

Peak 2, (off-white solid, 2.1 mg), Example 417: (1S,5R)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide or (1R,5S)-6,6-difluoro-N-(7-methoxy-4-(1-methyl-3-phenyl-1H-pyrazol-4-yl)pyrido[3,2-d]pyrimidin-6-yl)-3-methyl-3-azabicyclo[3.1.0]hexane-1-carboxamide. LCMS: m/z=492 [M+H]⁺; ¹H NMR (300 MHz, DMSO-d₆) δ: 10.53 (s, 1H), 9.19 (s, 1H), 8.87 (s, 1H), 7.76 (s, 1H), 7.50 (dd, 2H), 7.32 (dd, 3H), 4.04 (d, 6H), 2.99 (s, 3H), 2.51 (s, 3H), 2.29 (s, 2H).

### Example 418: (1R,3S,5R)—N-(7-methoxy-4-(1-methyl-3-phenyl-1H-pyrazol-4-yl)pyrido[3,2-d]pyrimidin-6-yl)-2-methyl-2-azabicyclo[3.1.0]hexane-3-carboxamide

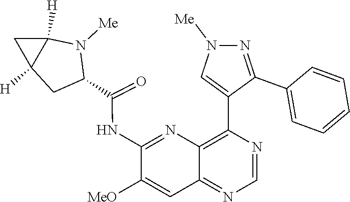 [SMILES: COc1cc2ncnc(-c3cn(C)nc3-c3ccccc3)c2nc1NC(=O)[C@@H]1C[C@H]2C[C@H]2N1C]

The title compound was prepared as a yellow solid from tert-butyl (1R,3S,5R)-3-((7-methoxy-4-(1-methyl-3-phenyl-1H-pyrazol-4-yl)pyrido[3,2-d]pyrimidin-6-yl)carbamoyl)-2-azabicyclo[3.1.0]hexane-2-carboxylate (Preparation 263) using an analogous 2-part procedure as described for Example 416. Prep-HPLC (YMC-Actus Triart C18 ExRS, 30×150 mm, 5 μm; 42-69% MeCN/H₂O (10 mM NH₄HCO₃+0.1% NH₄OH)) afforded the title compound. LCMS: m/z=456 [M+H]⁺; ¹H NMR (400 MHZ, DMSO-d₆) δ: 10.47 (s, 1H), 9.81 (s, 1H), 8.77 (s, 1H), 7.69 (s, 1H), 7.57-7.49 (m, 2H), 7.35 (qd, 3H), 4.02 (s, 3H), 3.98 (s, 3H), 2.96 (td, 1H), 2.88 (dd, 1H), 2.42 (s, 4H), 2.04 (ddd, 1H), 1.48 (dd, 1H), 0.73 (ddd, 1H), 0.22 (dt, 1H).

### Example 419: (1R,3R,5R)—N-(7-methoxy-4-(1-methyl-3-phenyl-1H-pyrazol-4-yl)pyrido[3,2-d]pyrimidin-6-yl)-2-methyl-2-azabicyclo[3.1.0]hexane-3-carboxamide

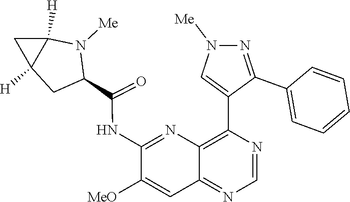 [SMILES: COc1cc2ncnc(-c3cn(C)nc3-c3ccccc3)c2nc1NC(=O)[C@H]1C[C@H]2C[C@H]2N1C]

The title compound was prepared as an off-white solid from tert-butyl (1R,3R,5R)-3-((7-methoxy-4-(1-methyl-3-phenyl-1H-pyrazol-4-yl)pyrido[3,2-d]pyrimidin-6-yl)carbamoyl)-2-azabicyclo[3.1.0]hexane-2-carboxylate (Preparation 264) using an analogous 2-part procedure as described for Example 416. Prep-HPLC (YMC-Actus Triart C18 ExRS, 30×150 mm, 5 μm; 41-71% MeCN/H₂O (10 mM NH₄HCO₃+0.1% NH₄OH)) afforded the title compound. LCMS: m/z=456 [M+H]⁺; ¹H NMR (400 MHz, DMSO-d₆) δ: 10.90 (s, 1H), 9.87 (s, 1H), 8.77 (s, 1H), 7.71 (s, 1H), 7.58-7.51 (m, 2H), 7.40-7.29 (m, 3H), 4.14 (s, 3H), 4.00 (s, 3H), 3.82 (dd, 1H), 2.73 (s, 1H), 2.59 (s, 3H), 2.44 (s, 1H), 2.39-2.23 (m, 2H), 1.54 (dq, 1H), 0.52 (q, 1H).

### Example 420:3-(dimethylamino)-N-(7-ethoxy-4-(1-methyl-3-phenyl-1H-pyrazol-4-yl)pyrido[3,2-d]pyrimidin-6-yl)bicyclo[1.1.1]pentane-1-carboxamide

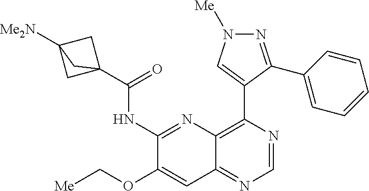 [SMILES: CCOc1cc2ncnc(-c3cn(C)nc3-c3ccccc3)c2nc1NC(=O)C12CC(N(C)C)(C1)C2]

The title compound was prepared as a white solid from 6-chloro-7-ethoxy-4-(1-methyl-3-phenyl-1H-pyrazol-4-yl)pyrido[3,2-d]pyrimidine (Preparation 231) and tert-butyl (3-carbamoylbicyclo[1.1.1]pentan-1-yl)carbamate (Preparation 63) using an analogous 3-part procedure as descried for Example 377 and 378. The crude product was purified by XBridge Shield RP18 OBD, 30×150 mm, 5 μm; 9-57% MeCN/H₂O (10 mM NH₄HCO₃+0.1% NH₄OH)). LCMS: m/z=484 [M+H]⁺; ¹H NMR (300 MHz, DMSO-d₆) δ: 9.47 (s, 1H), 9.39 (s, 1H), 8.82 (s, 1H), 7.70 (s, 1H), 7.51 (dd, 2H), 7.42-7.19 (m, 3H), 4.36 (q, 2H), 4.00 (s, 3H), 2.16 (s, 6H), 2.08 (s, 6H), 1.47 (t, 3H).

### Example 421: N-(4-(3-(2,4-difluorophenyl)-1-methyl-1H-pyrazol-4-yl)-7-methoxypyrido[3,2-d]pyrimidin-6-yl)bicyclo[1.1.1]pentane-1-carboxamide

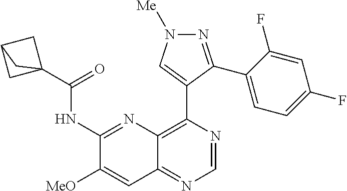 [SMILES: COc1cc2ncnc(-c3cn(C)nc3-c3ccc(F)cc3F)c2nc1NC(=O)C12CC(C1)C2]

The title compound was prepared as an off-white solid (7.6 mg, 20%) from 4-(3-(2,4-difluorophenyl)-1-methyl-1H-pyrazol-4-yl)-7-methoxypyrido[3,2-d]pyrimidin-6-amine (Preparation 217) and bicyclo[1.1.1]pentane-1-carboxylic acid using an analogous method to that describe in Part 1 of Example 381. The crude product was purified by Prep-HPLC (XBridge Shield RP18 OBD, 30×150 mm, 5 μm; 18-62% MeCN/H₂O (10 mM NH₄HCO₃+0.1% NH₄OH)). LCMS: m/z=463 [M+H]⁺; ¹H NMR (400 MHZ, DMSO-d₆) δ: 9.66 (s, 1H), 9.46 (s, 1H), 8.77 (s, 1H), 7.71 (s, 1H), 7.54 (td, 1H), 7.20 (td, 1H), 7.18-7.08 (m, 1H), 4.09 (s, 3H), 4.01 (s, 3H), 2.53 (s, 1H), 2.20 (s, 6H).

### Example 422: N-(4-(3-(3-hydroxyphenyl)-1-methyl-1H-pyrazol-4-yl)-7-methoxypyrido[3,2-d]pyrimidin-6-yl)-1-(trifluoromethyl)cyclopropane-1-carboxamide

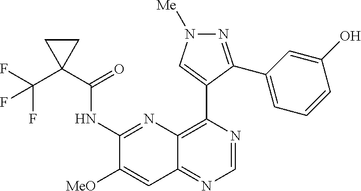 [SMILES: COc1cc2ncnc(-c3cn(C)nc3-c3cccc(O)c3)c2nc1NC(=O)C1(C(F)(F)F)CC1]

The title compound was prepared as a white solid (31.9 mg, 16%) from 3-(4-(6-amino-7-methoxypyrido[3,2-d]pyrimidin-4-yl)-1-methyl-1H-pyrazol-3-yl)phenol (Preparation 219) and 1-(trifluoromethyl)cyclopropane-1-carboxylic acid using an analogous method to that described for Example 381. The crude product was purified by Prep-HPLC (XSelect CSH C18 OBD, 19×250 mm, 5 μm; 35-50% MeCN/H₂O (0.1% HCO₂H)). LCMS: m/z=485 [M+H]⁺; ¹H NMR (300 MHZ, DMSO-d₆) δ: 9.82 (s, 1H), 9.26 (s, 1H), 9.05 (s, 1H), 8.93 (s, 1H), 7.79 (s, 1H), 7.10 (t, 1H), 6.91 (dp, 2H), 6.70 (ddd, 1H), 4.08 (s, 3H), 3.98 (s, 3H), 1.62 (s, 2H), 1.49-1.43 (m, 2H).

### Example 423 and 424: (1S,5S)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide and (1R,5R)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide

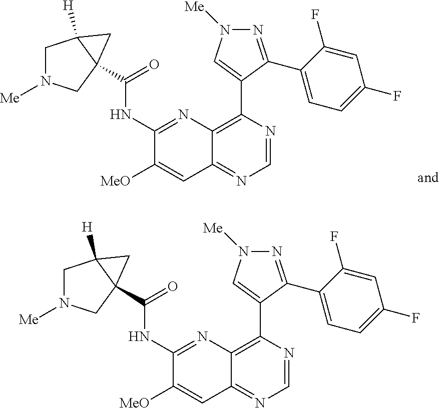 [SMILES: COc1cc2ncnc(-c3cn(C)nc3-c3ccc(F)cc3F)c2nc1NC(=O)[C@@]12C[C@@H]1CN(C)C2 ; COc1cc2ncnc(-c3cn(C)nc3-c3ccc(F)cc3F)c2nc1NC(=O)[C@]12C[C@H]1CN(C)C2]

Part 1. tert-butyl 1-((4-(3-(2,4-difluorophenyl)-1-methyl-1H-pyrazol-4-yl)-7-methoxypyrido[3,2-d]pyrimidin-6-yl)carbamoyl)-3-azabicyclo[3.1.0]hexane-3-carboxylate was prepared as an off-white solid (7.6 mg, 20%) from 4-(3-(2,4-difluorophenyl)-1-methyl-1H-pyrazol-4-yl)-7-methoxypyrido[3,2-d]pyrimidin-6-amine (Preparation 217) and 3-(tert-butoxycarbonyl)-3-azabicyclo[3.1.0]hexane-1-carboxylic acid using an analogous method to that describe in Part 1 of Example 381. LCMS: m/z=578 [M+H]⁺.

Part 2. N-(4-(3-(2,4-difluorophenyl)-1-methyl-1H-pyrazol-4-yl)-7-methoxypyrido[3,2-d]pyrimidin-6-yl)-3-azabicyclo[3.1.0]hexane-1-carboxamide was prepared as a yellow solid (16 mg, 80%) from tert-butyl 1-((4-(3-(2,4-difluorophenyl)-1-methyl-1H-pyrazol-4-yl)-7-methoxypyrido[3,2-d]pyrimidin-6-yl)carbamoyl)-3-azabicyclo[3.1.0]hexane-3-carboxylate (Part 1) using an analogous method to that described for Example 390.

Part 3. N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide was prepared as an off-white solid from N-(4-(3-(2,4-difluorophenyl)-1-methyl-1H-pyrazol-4-yl)-7-methoxypyrido[3,2-d]pyrimidin-6-yl)-3-azabicyclo[3.1.0]hexane-1-carboxamide (Part 2) using an analogous method to that described for Example 390.

Part 4. N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide (Part 3) was separated by chiral-HPLC (Chiral ART Cellulose-SC, 20×250 mm, 5 μm; 10% IPA/(3:1 Hex/DCM (+0.5% 2M NH₃—MeOH))) to afford the title compounds.

Peak 1, (white solid, 2.3 mg), Example 423: (1S,5S)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1R,5R)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=492 [M+H]⁺; ¹H NMR (400 MHZ, DMSO-d₆) δ: 9.54 (s, 1H), 9.26 (s, 1H), 8.78 (s, 1H), 7.70 (s, 1H), 7.55 (q, 1H), 7.24-7.12 (m, 1H), 7.12 (d, 1H), 4.08 (s, 3H), 4.03 (s, 1H), 4.01 (s, 3H), 3.25 (s, 2H), 2.95 (d, 1H), 2.70 (s, 1H), 2.31 (s, 3H), 2.13 (s, 1H), 1.41 (d, 1H), 1.33 (d, 1H), 1.24 (s, 1H).

Peak 2, (white solid, 3.7 mg), Example 424: (1R,5R)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1S,5S)—N-(4-(3-(2,4-difluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=492 [M+H]⁺; ¹H NMR (400 MHZ, DMSO-d₆) δ: 9.54 (s, 1H), 9.26 (s, 1H), 8.78 (s, 1H), 7.70 (s, 1H), 7.55 (q, 1H), 7.24-7.08 (m, 2H), 4.08 (s, 3H), 4.01 (s, 3H), 2.96 (d, 1H), 2.70 (d, 1H), 2.43 (s, 2H), 2.31 (s, 3H), 1.41 (t, 1H), 1.33 (dd, 1H), 1.23 (s, 2H).

### Example 425 and 426: (1R,5S)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide and (1S,5R)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide

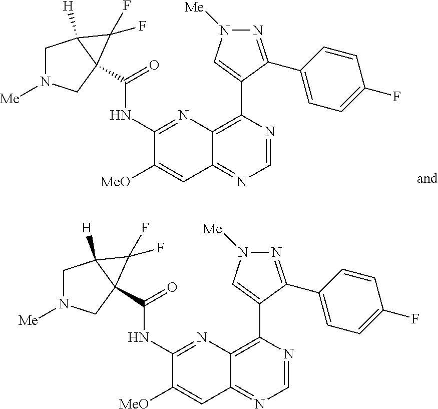 [SMILES: COc1cc2ncnc(-c3cn(C)nc3-c3ccc(F)cc3)c2nc1NC(=O)[C@]12CN(C)C[C@H]1C2(F)F ; COc1cc2ncnc(-c3cn(C)nc3-c3ccc(F)cc3)c2nc1NC(=O)[C@@]12CN(C)C[C@@H]1C2(F)F]

The title compounds were prepared from 4-(3-(4-fluorophenyl)-1-methyl-1H-pyrazol-4-yl)-7-methoxypyrido[3,2-d]pyrimidin-6-amine (Preparation 218) and 3-(tert-butoxycarbonyl)-6,6-difluoro-3-azabicyclo[3.1.0]hexane-1-carboxylic acid using an analogous 4-part method as described for Example 423 and 424. The racemate was purified by Chiral-HPLC (Chiralpak IH, 20×250 mm, 5 μm; 10% EtOH/MtBE (0.5% 2M NH₃—MeOH)) to afford:

Peak 1, (white solid, 40.3 mg), Example 425: (1R,5S)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1S,5R)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=510 [M+H]⁺; ¹H NMR (300 MHz, DMSO-d₄) δ: 10.48 (s, 1H), 9.24 (s, 1H), 8.88 (s, 1H), 7.75 (s, 1H), 7.56 (dd, 2H), 7.25-6.93 (m, 2H), 4.04 (d, 6H), 3.30 (d, 1H), 3.06-2.80 (m, 4H), 2.24 (s, 3H).

Peak 2, (white solid, 36.6 mg), Example 426: (1S,5R)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide or (1R,5S)-6,6-difluoro-N-(4-(3-(4-fluorophenyl)-1-methyl-1H-pyrazol-4-yl)-7-methoxypyrido[3,2-d]pyrimidin-6-yl)-3-methyl-3-azabicyclo[3.1.0]hexane-1-carboxamide. LCMS: m/z=510 [M+H]⁺; ¹H NMR (300 MHz, DMSO-d₄) δ: 10.48 (s, 1H), 9.24 (s. 1H), 8.88 (s, 1H), 7.75 (s, 1H), 7.56 (dd, 2H), 7.25-6.93 (m, 2H), 4.04 (d, 6H), 3.30 (d, 1H), 3.06-2.80 (m, 4H), 2.24 (s, 3H).

### Example 427 and 428: N-(7-methoxy-4-(1-methyl-3-phenyl-1H-pyrazol-4-yl)pyrido[3,2-d]pyrimidin-6-yl)-5-methyl-1-(trifluoromethyl)-1H-pyrazole-4-carboxamide and N-(7-methoxy-4-(1-methyl-3-phenyl-1H-pyrazol-4-yl)pyrido[3,2-d]pyrimidin-6-yl)-3-methyl-1-(trifluoromethyl)-1H-pyrazole-4-carboxamide

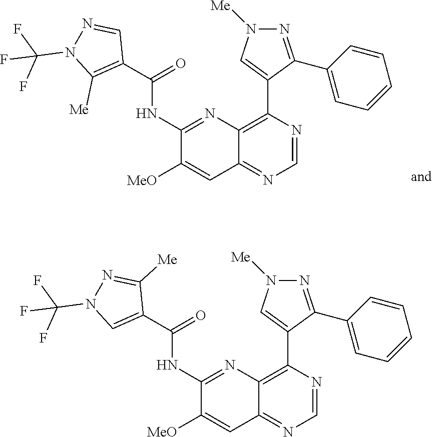 [SMILES: COc1cc2ncnc(-c3cn(C)nc3-c3ccccc3)c2nc1NC(=O)c1cnn(C(F)(F)F)c1C ; COc1cc2ncnc(-c3cn(C)nc3-c3ccccc3)c2nc1NC(=O)c1cn(C(F)(F)F)nc1C]

A solution of 3-methyl-1-(trifluoromethyl)-1H-pyrazole-4-carboxylic acid and 5-methyl-1-(trifluoromethyl)-1H-pyrazole-4-carboxylic acid (Preparation 85, 100 mg, 0.515 mmol), HOBT (104 mg, 0.773 mmol) and EDCl (148 mg, 0.773 mmol) in DCM (10 mL) was stirred at rt for 1 h. NH₃ aq (2 mL) was added and the reaction stirred for a further hour and then concentrated. The residue was purified by prep-TLC with DCM:MeOH=20:1 to give a mixture of 3-methyl-1-(trifluoromethyl)-1H-pyrazole-4-carboxamide and 5-methyl-1-(trifluoromethyl)-1H-pyrazole-4-carboxamide (80 mg, yield=80.4%) as a white solid.

A mixture of 3-methyl-1-(trifluoromethyl)-1H-pyrazole-4-carboxamide and 5-methyl-1-(trifluoromethyl)-1H-pyrazole-4-carboxamide (80 mg, 0.414 mmol), 6-chloro-7-methoxy-4-(1-methyl-3-phenyl-1H-pyrazol-4-yl)pyrido[3,2-d]pyrimidine (Preparation 242, 145 mg, 0.414 mmol), K₂CO: (86 mg, 0.621 mmol), XPhos (30 mg, 0.062 mmol) and Pd₂(dba)₃ (57 mg, 0.062 mmol) in dioxane (5 mL) was stirred at 100° C. for 3 h under N₂. The reaction mixture was diluted with water and extracted with EtOAc. The combined organics were evaporated to dryness in vacuo and the residue purified by silica gel chromatography (5% MeOH/DCM) followed by prep-HPLC (XBridge Prep OBD C18 Column, 30×150 mm, 5 μm; 34-54% MeCN/H₂O (10 mM NH₄HCO₃+0.1% NH₄OH)) to afford:

Peak 1, white solid (35.4 mg, 17%). Example 427, N-(7-methoxy-4-(1-methyl-3-phenyl-1H-pyrazol-4-yl)pyrido[3,2-d]pyrimidin-6-yl)-5-methyl-1-(trifluoromethyl)-1H-pyrazole-4-carboxamide. LCMS: m/z=509 [M+H]⁺; ¹H NMR (400 MHZ, DMSO-d₆) δ: 10.53 (s, 1H), 9.15 (s, 1H), 8.89 (s, 1H), 8.46 (s, 1H), 7.79 (s, 1H), 7.50 (dd, 2H), 7.32 (qd, 3H), 4.08 (s, 3H), 3.99 (s, 3H), 3.17 (d, 1H), 2.77 (q, 3H).

Peak 2, white solid (12.2 mg, 6%). Example 428, N-(7-methoxy-4-(1-methyl-3-phenyl-1H-pyrazol-4-yl)pyrido[3,2-d]pyrimidin-6-yl)-3-methyl-1-(trifluoromethyl)-1H-pyrazole-4-carboxamide. LCMS: m/z=509 [M+H]⁺; ¹H NMR (400 MHZ, DMSO-d₆) δ: 10.41 (s, 1H), 9.27 (s, 1H), 9.21 (s, 1H), 8.87 (s, 1H), 7.78 (s, 1H), 7.52 (dd, 2H), 7.33 (dd, 3H), 4.10 (s, 3H), 3.99 (s, 3H), 2.54 (s, 3H).

### Example 429 and 430: (R)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide and (S)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide

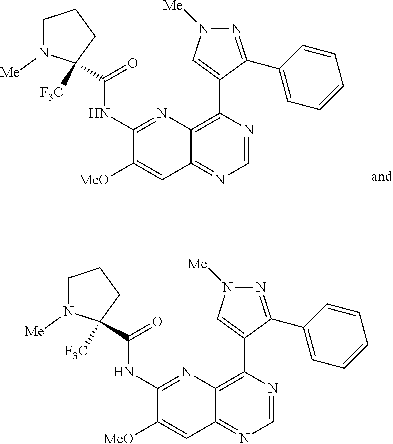 [SMILES: COc1cc2ncnc(-c3cn(C)nc3-c3ccccc3)c2nc1NC(=O)[C@@]1(C(F)(F)F)CCCN1C ; COc1cc2ncnc(-c3cn(C)nc3-c3ccccc3)c2nc1NC(=O)[C@]1(C(F)(F)F)CCCN1C]

Part 1. A mixture of 6-chloro-7-methoxy-4-(1-methyl-3-phenyl-1H-pyrazol-4-yl)pyrido[3,2-d]pyrimidine (Preparation 242, 180 mg, 0.511 mmol), 2-(trifluoromethyl) pyrrolidine-2-carboxamide (Preparation 78, 93.0 mg, 0.511 mmol), XPhos (58.9 mg, 0.102 mmol) and Pd₂(dba)₃ (46.7 mg, 0.051 mmol) in dioxane (10 mL) was stirred at 100° C. for 16 h under N₂. The reaction mixture was diluted with EtOAc (100 mL) and washed with brine (2×50 mL). The combined organics were dried (Na₂SO₄) and evaporated to dryness in vacuo and the residue was purified by prep-TLC (20% EtOAc/PE) to afford N-(7-methoxy-4-(1-methyl-3-phenyl-1H-pyrazol-4-yl)pyrido[3,2-d]pyrimidin-6-yl)-2-(trifluoromethyl) pyrrolidine-2-carboxamide as a yellow solid (100 mg, 39%). LCMS: m/z=498 [M+H]⁺.

Part 2. NaCNBH₃ (15.2 mg, 0.402 mmol) was added to formaldehyde (162 mg, 2.01 mmol) and N-(7-methoxy-4-(1-methyl-3-phenyl-1H-pyrazol-4-yl)pyrido[3,2-d]pyrimidin-6-yl)-2-(trifluoromethyl) pyrrolidine-2-carboxamide (100 mg, 0.201 mmol) in MeOH at rt and the resulting mixture was stirred at rt for 16 h. The mixture was evaporated to dryness in vacuo and the residue purified by prep-TLC (25:1 DCM/MeOH) to afford N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide as a yellow solid (50 mg, 47%)). LCMS: m/z=512 [M+H]⁺.

Part 3. N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide was separated by chiral-HPLC (Lux 5 μm Cellulose-4, 21.2×250 mm, 5 μm; 15% EtOH/Hex (0.5% 2M NH₃—MeOH)) to afford the title compounds.

Peak 1, yellow solid (15.8 mg). Example 429, (R)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide or (S)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide. LCMS: m/z=512 [M+H]⁺; ¹H NMR (300 MHZ, DMSO-d₆) δ: 9.61 (s, 1H), 8.82 (s, 1H), 7.75 (s, 1H), 7.53 (dd, 2H), 7.34 (dd, 3H), 4.12 (s, 3H), 3.99 (s, 3H), 2.91 (q, 1H), 2.66 (d, 3H), 2.49-2.29 (m, 2H), 1.95 (d, 2H), 1.44 (s, 6H), 0.91 (s, 1H), 0.80 (dd, 1H).

Peak 2, yellow solid (13.4 mg). Example 430, (S)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide or (R)—N-(7-methoxy-4-(1-methyl-3-phenyl-1H-pyrazol-4-yl)pyrido[3,2-d]pyrimidin-6-yl)-1-methyl-2-(trifluoromethyl) pyrrolidine-2-carboxamide. LCMS: m/z=512 [M+H]⁺; ¹H NMR (300 MHZ, DMSO-d₆) δ: 10.67 (s, 1H), 9.61 (s, 1H), 8.82 (s, 1H), 7.75 (s, 1H), 7.53 (dd, 2H), 7.38-7.30 (m, 3H), 4.12 (s, 3H), 3.99 (s, 3H), 2.91 (q, 1H), 2.66 (s, 3H), 2.40 (dq, 2H), 1.95 (q, 2H), 1.23 (s, 3H), 0.80 (dd, 1H).

### Example 431 and 432: (1R,5R)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide and (1S,5S)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide

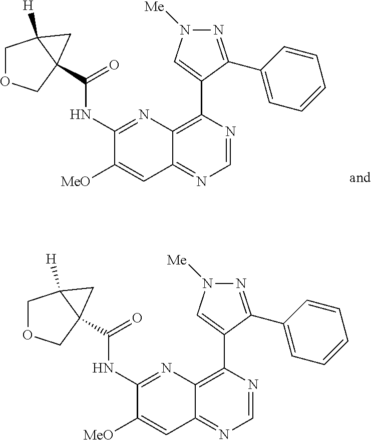 [SMILES: COc1cc2ncnc(-c3cn(C)nc3-c3ccccc3)c2nc1NC(=O)[C@@]12COC[C@@H]1C2 ; COc1cc2ncnc(-c3cn(C)nc3-c3ccccc3)c2nc1NC(=O)[C@]12COC[C@H]1C2]

N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide was prepared as a white solid (40 mg, 16%) from 6-chloro-7-methoxy-4-(1-methyl-3-phenyl-1H-pyrazol-4-yl)pyrido[3,2-d]pyrimidine (Preparation 242) and 3-oxabicyclo[3.1.0]hexane-1-carboxamide (Preparation 64) using an analogous method to that described for Example 232. LCMS: m/z=443 [M+H]⁺. The compound was further purified by chiral-HPLC (Column: CHIRALPAK 1H, 5*25 cm, 5 μm; Mobile Phase A: Hex (0.5% 2M NH₃—MeOH), Mobile Phase B: EtOH: Flow rate: 20 mL/min; Gradient: 15% isocratic) to give:

Peak 1, off-white solid (25.1 mg). Example 431: (1R,5R)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide or (1S,5S)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide. LCMS: m/z=443 [M+H]⁺; ¹H NMR (300 MHz, DMSO-d₆) δ: 9.58 (s, 1H), 9.19 (s, 1H), 8.87 (s, 1H), 7.75 (s, 1H), 7.55-7.45 (m, 2H), 7.40-7.27 (m, 3H), 4.10-3.97 (m, 8H), 3.94-3.79 (d, 2H), 2.35 (ddd, 1H), 1.54 (dd, 1H), 0.97 (t, 1H).

Peak 2, off-white solid (22.1 mg). Example 432: (1S,5S)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide or (1R,5R)—N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide. LCMS: m/z=443 [M+H]⁺; ¹H NMR (300 MHz, DMSO-d₆) δ: 9.58 (s, 1H), 9.19 (s, 1H), 8.87 (s, 1H), 7.75 (s, 1H), 7.57-7.45 (m, 2H), 7.33 (dt, 3H), 4.10-3.97 (m, 8H), 3.79 (d, 2H), 2.40-2.29 (m, 1H), 1.54 (dd, 1H), 0.97 (t, 1H).

### Example 433:3-fluoro-N-(7-methoxy-4-(1-methyl-3-phenyl-1H-pyrazol-4-yl)pyrido[3,2-d]pyrimidin-6-yl)-1-methylpyrrolidine-3-carboxamide

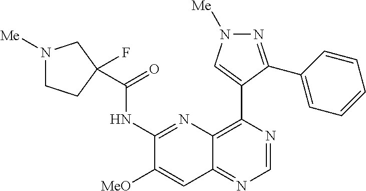 [SMILES: COc1cc2ncnc(-c3cn(C)nc3-c3ccccc3)c2nc1NC(=O)C1(F)CCN(C)C1]

Part 1. 3-fluoro-N-(7-methoxy-4-(1-methyl-3-phenyl-1H-pyrazol-4-yl)pyrido[3,2-d]pyrimidin-6-yl) pyrrolidine-3-carboxamide was prepared as a white solid (100 mg, 52%) from 6-chloro-7-methoxy-4-(1-methyl-3-phenyl-1H-pyrazol-4-yl)pyrido[3,2-d]pyrimidine (Preparation 242) and 3-fluoropyrrolidine-3-carboxamide using an analogous method to that described for Example 232. LCMS: m/z=446 [M+H]⁺.

Part 2. A mixture of 3-fluoro-N-(7-methoxy-4-(1-methyl-3-phenyl-1H-pyrazol-4-yl)pyrido[3,2-d]pyrimidin-6-yl) pyrrolidine-3-carboxamide (Part 1, 100 mg, 0.223 mmol) and formaldehyde solution (0.1 mL) in MeOH (2 mL) and AcOH (0.2 mL) at rt was stirred for 20 min before NaCNBH₃ (28.0 mg, 0.446 mmol) was added at 0° C. and the reaction mixture stirred at rt for 2 h. The resulting solution was extracted with EtOAc (3×30 mL) and the combined organics were dried (Na₂SO₄) and evaporated to dryness. The residue was purified by prep-HPLC (YMC-Actus Triart C18 ExRS, 30×150 mm, 5 μm; 29-51% MeCN/H₂O (10 mmol/L NH₄HCO₃+0.1% NH₃·H₂O)) afforded title compound as a white solid (13.1 mg, 12%). LCMS: m/z=462 [M+H]⁺; ¹H NMR (400 MHz, DMSO-d₆) δ: 10.17 (d, 1H), 9.52 (s, 1H), 8.84 (s, 1H), 7.77 (s, 1H), 7.55-7.50 (m, 2H), 7.34 (dd, 3H), 4.10 (s, 3H), 4.00 (s, 3H), 3.04 (d, 2H), 2.90 (d, 1H), 2.64 (d, 2H), 2.39 (s, 3H), 2.30 (s, 1H).

### Example 434: N-(7-(2-(dimethylamino) ethoxy)-4-(1-methyl-3-phenyl-1H-pyrazol-4-yl)pyrido[3,2-d]pyrimidin-6-yl)-1-(trifluoromethyl)cyclopropane-1-carboxamide

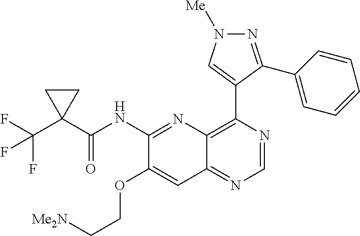 [SMILES: CN(C)CCOc1cc2ncnc(-c3cn(C)nc3-c3ccccc3)c2nc1NC(=O)C1(C(F)(F)F)CC1]

Part 1. L-Selectride (121 mg, 640 μmol) was added to a solution of N-(7-methoxy-4-(1-methyl-3-phenyl-1H-pyrazol-4-yl)pyrido[3,2-d]pyrimidin-6-yl)-1-(trifluoromethyl)cyclopropane-1-carboxamide (Example 361, 150 mg, 0.320 mmol) in THF (10 mL) at 0° C. and the reaction mixture stirred at 0° C. for 15 min and then at 80° C. for 4 h. The reaction mixture was poured into ice water and the reaction mixture was concentrated under vacuum. The mixture was diluted with EtOAc (100 mL) and washed with brine (2×50 mL). The combined organics were dried (Na₂SO₄) and evaporated to dryness in vacuo and the residue was purified by column chromatography (10:1 DCM/MeOH) to afford N-(7-hydroxy-4-(1-methyl-3-phenyl-1H-pyrazol-4-yl)pyrido[3,2-d]pyrimidin-6-yl)-1-(trifluoromethyl)cyclopropane-1-carboxamide as a yellow solid (90 mg, 62%). LCMS: m/z=455 [M+H]⁺.

Part 2. To a mixture of N-(7-hydroxy-4-(1-methyl-3-phenyl-1H-pyrazol-4-yl)pyrido[3,2-d]pyrimidin-6-yl)-1-(trifluoromethyl)cyclopropane-1-carboxamide (Part 1, 70 mg, 0.154 mmol) and 2-(dimethyl amino) ethan-1-ol (41.1 mg, 0.462 μmol) in THF (5 mL) was added sequentially PPh₃ (173 mg, 0.66 mmol) and DIAD (93 mg, 0.462 mmol) at ice bath temperature and the reaction mixture stirred at 25° C. for 16 h. under N₂. The mixture was evaporated to dryness in vacuo and the residue purified by prep-TLC (10:1 EtOAc/MeOH) and prep-HPLC (Selects CSH C18 OBD, 30×150 mm, 5 μm, 15-25% MeCN/H₂O (0.1% HCO₂H) to give the title compound as an off-white solid (16.3 mg, 14%). LCMS: m/z=526 [M+H]⁺; ¹H NMR (300 MHz, DMSO-d₆) δ: 9.19 (s, 1H), 8.87 (s, 1H), 7.81 (s, 1H), 7.49 (d, 2H), 7.32 (d, 3H), 4.38 (s, 1H), 3.99 (s, 3H), 2.73 (t, 2H), 2.26 (s, 6H), 1.63 (s, 2H), 1.59-1.29 (m, 2H).

### Example 435: N-(7-(2-(dimethylamino) ethoxy)-4-(1-methyl-3-phenyl-1H-pyrazol-4-yl)pyrido[3,2-d]pyrimidin-6-yl)-3-oxabicyclo[3.1.0]hexane-1-carboxamide

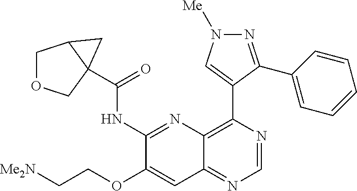 [SMILES: CN(C)CCOc1cc2ncnc(-c3cn(C)nc3-c3ccccc3)c2nc1NC(=O)C12COCC1C2]

Part 1. N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide was prepared as a yellow solid (120 mg, 16%) from 6-chloro-7-methoxy-4-(1-methyl-3-phenyl-1H-pyrazol-4-yl)pyrido[3,2-d]pyrimidine (Preparation 242) and 3-oxabicyclo[3.1.0]hexane-1-carboxamide (Preparation 64) using an analogous method to that described for Example 232. LCMS: m/z=443 [M+H]⁺.

Part 2. The title compound was prepared from N-(7-methoxy-4-(1-methyl-3-phenyl-1H-pyrazol-4-yl)pyrido[3,2-d]pyrimidin-6-yl)-3-oxabicyclo[3.1.0]hexane-1-carboxamide (Part 1) using an analogous method to that described Example 434. The crude was purified by prep-HPLC (Bridge OBD C18, 30×150 mm, 5 μm; 44-69% MeCN/H₂O (10 mM NH₄HCO₃+0.1% NH₄OH)) to afford the title compound (2.1 mg, 2.3%). LCMS: m/z=500 [M+H]⁺; ¹H NMR (300 MHz, DMSO-d₆) δ: 9.35 (s, 1H), 9.09 (s, 1H), 8.74 (s, 1H), 7.66 (s, 1H), 7.55-7.29 (m, 2H), 7.20 (m, 3H), 4.23 (d, 2H), 4.11-3.76 (m, 5H), 3.76-3.57 (m, 2H), 2.63 (d, 2H), 2.15 (s, 7H), 1.41 (m, 1H), 1.11 (s, 1H), 0.86 (t, 1H)

### Example 436:1-methoxy-N-(7-methoxy-4-(1-methyl-3-phenyl-1H-pyrazol-4-yl)pyrido[3,2-d]pyrimidin-6-yl)cyclopropane-1-carboxamide

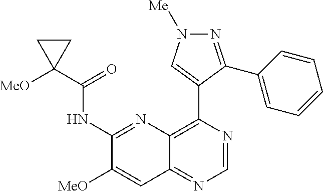 [SMILES: COc1cc2ncnc(-c3cn(C)nc3-c3ccccc3)c2nc1NC(=O)C1(OC)CC1]

To a solution of 6-chloro-7-methoxy-4-(1-methyl-3-phenyl-1H-pyrazol-4-yl)pyrido[3,2-d]pyrimidine (Preparation 242, 50 mg, 0.142 mmol) in DMF (1.5 mL) was added Pd₂(dba)₃ (6.51 mg, 7.11 μmol), BINAP (4.43 mg, 7.11 μmol), XPhos Pd G3 (5.94 mg, 7.11 μmol), NaO^{t}Bu (41 mg, 0.426 mmol) and 1-methoxycyclopropane-1-carboxamide and the mixture stirred at rt for 16 h. DCM and water were added and the organics separated. The aqueous layer was re-extracted with DCM and the combined organics dried and evaporated to dryness. The residue was purified by reverse phase ISCO C18 chromatography to afford the title compound (46 mg, 75%). LCMS: m/z=431 [M+H]⁺; ¹H NMR (300 MHz, DMSO-d₆) δ: 9.88 (d, 1H), 8.80 (d, 1H), 7.65 (s, 1H), 7.58-7.56 (m, 2H), 7.45-7.35 (m, 3H), 4.25 (s, 3H), 4.08 (s, 3H), 3.54 (s, 3H), 1.48 (q, 2H), 1.37 (q, 2H).

### Example 437: N-(7-methoxy-4-(2-methyl-4-phenylthiazol-5-yl)pyrido[3,2-d]pyrimidin-6-yl)cyclopropanecarboxamide

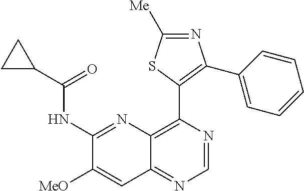 [SMILES: COc1cc2ncnc(-c3sc(C)nc3-c3ccccc3)c2nc1NC(=O)C1CC1]

The title compound was prepared as a yellow solid (3.9 mg, 8%) from 5-(6-chloro-7-methoxypyrido[3,2-d]pyrimidin-4-yl)-2-methyl-4-phenylthiazole (Preparation 241) and cyclopropanecarboxamide using an analogous method to that described for Example 232. The crude was purified by prep-HPLC (YMC-Actus Triart C18 ExRS, 30×150 mm, 5 μm; 30-54% MeCN/H₂O (10 mM NH₄HCO₃+0.1% NH₄OH). LCMS: m/z=418 [M+H]⁺; ¹H NMR (400 MHZ, DMSO-d₆) δ: 10.11 (s, 1H), 9.02 (s, 1H), 7.75 (s, 1H), 7.36-7.32 (m, 2H), 7.23 (d, 3H), 4.05 (s, 3H), 2.78 (s, 3H), 2.26 (dq, 1H), 0.88-0.76 (m, 4H).

### Example 438:4-(1-methyl-3-phenyl-1H-pyrazol-4-yl)-7-(1-(1-methylpiperidin-4-yl)-1H-pyrazol-4-yl)quinazoline

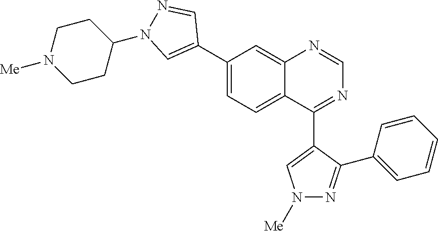 [SMILES: CN1CCC(n2cc(-c3ccc4c(-c5cn(C)nc5-c5ccccc5)ncnc4c3)cn2)CC1]

The title compound was obtained as a white solid, 20 mg, 15% yield, from 4-chloro-7-(1-(1-methylpiperidin-4-yl)-1H-pyrazol-4-yl)quinazoline (Preparation 270) and 1-methyl-3-phenyl-4-(4,4,5,5-tetramethyl-1,3,2-dioxaborolan-2-yl)-1H-pyrazole following a similar procedure to that described in Example 20. LCMS m/z=450 [M+H]⁺. ¹H NMR (300 MHz, DMSO-d₆) δ 9.16 (s, 1H), 8.58 (s, 1H), 8.28 (s, 1H), 8.19 (d, 2H), 7.89-7.73 (m, 2H), 7.32 (dt, 2H), 7.22 (p, 3H), 4.26-4.10 (m, 1H), 4.04 (s, 3H), 2.89 (d, 2H), 2.24 (s, 3H), 2.18-1.88 (m, 6H).

### Example 439:5-fluoro-7-(1-methyl-1H-pyrazol-4-yl)-4-(1-methyl-3-phenyl-1H-pyrazol-4-yl)quinazoline

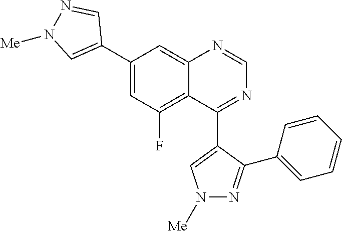 [SMILES: Cn1cc(-c2cc(F)c3c(-c4cn(C)nc4-c4ccccc4)ncnc3c2)cn1]

The title compound was obtained as an off-white solid, 50 mg, 17%, from 4-chloro-5-fluoro-7-(1-methyl-1H-pyrazol-4-yl)quinazoline (Preparation 273) and 1-methyl-3-phenyl-4-(4,4,5,5-tetramethyl-1,3,2-dioxaborolan-2-yl)-1H-pyrazole following a similar procedure to that described in Example 20. LCMS m/z=385 [M+H]⁺. ¹H NMR (DMSO-d₆, 400 MHz): ppm δ=9.20 (s, 1H), 8.49 (s, 1H), 8.21-8.13 (m, 2H), 8.06 (d, 1H), 7.64 (dd, 1H), 7.26 (dd, 2H), 7.19 (dq, 3H), 4.00 (s, 3H), 3.89 (s, 3H).

### Example 440:4-(3-(3-chlorophenyl)-1H-pyrazol-4-yl)-7-(1-methyl-1H-pyrazol-4-yl)quinazoline

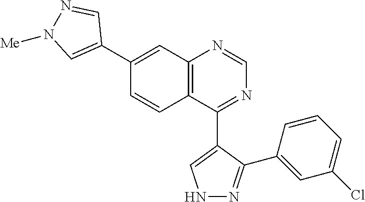 [SMILES: Cn1cc(-c2ccc3c(-c4c[nH]nc4-c4cccc(Cl)c4)ncnc3c2)cn1]

Part 1:3-(3-Chlorophenyl)-1-(tetrahydro-2H-pyran-2-yl)-4-(4,4,5,5-tetramethyl-1,3,2-dioxaborolan-2-yl)-1H-pyrazole (Preparation 47, 100 mg, 0.361 mmol) in DMSO (1 mL), 4-chloro-7-(1-methyl-1H-pyrazol-4-yl)quinazoline (Preparation 266, 105 mg, 0.433 mmol), Pd(PPh₃)₄ (41.7 mg, 0.0361 mmol) and K₃PO₄ (153 mg, 722 μmol) were added to an 8-mL sealed tube. The reaction mixture was stirred at 100° C. for 16 h. Water was added and the reaction mixture was extracted with EtOAc. The combined organic extracts were washed with brine then evaporated under reduced pressure. The crude product was purified by TLC with DCM/MeOH (20/1) to give 60 mg of 4-(3-(3-chlorophenyl)-1-(tetrahydro-2H-pyran-2-yl)-1H-pyrazol-4-yl)-7-(1-methyl-1H-pyrazol-4-yl)quinazoline as an off-white solid.

Part 2: TFA (1 mL) was added to a solution of 4-(3-(3-chlorophenyl)-1-(tetrahydro-2H-pyran-2-yl)-1H-pyrazol-4-yl)-7-(1-methyl-1H-pyrazol-4-yl)quinazoline (60 mg, 0.128 mmol) in DCM (4 mL) and the reaction was stirred at rt for 2 h. The reaction mixture was evaporated and purified by Prep-HPLC Column: XBridge Shield RP18 OBD Column, 30*150 mm, 5 um; Mobile Phase A: Water (0.05% NH₃H₂O), Mobile Phase B: MeCN; Flow rate: 60 mL/min; Gradient: 32% B to 40% B in 7 min, to give the title compound (17 mg) as a white solid. LCMS m/z=387 [M+H]⁺. ¹H NMR (400 MHz, DMSO-d₆) δ 13.65 (s, 1H), 9.16 (s, 1H), 8.47 (s, 1H), 8.17 (dd, J=12.1, 1.2 Hz, 2H), 7.94-7.81 (m, 2H), 7.56-7.46 (m, 1H), 7.37-7.18 (m, 4H), 3.90 (s, 3H).

### Example 441:7-(1-methyl-1H-pyrazol-4-yl)-4-(3-phenyl-1H-pyrazol-4-yl)quinazoline

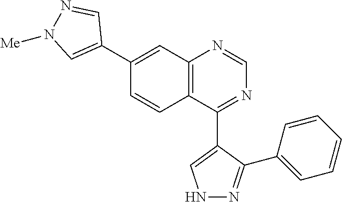 [SMILES: Cn1cc(-c2ccc3c(-c4c[nH]nc4-c4ccccc4)ncnc3c2)cn1]

7-(1-Methyl-1H-pyrazol-4-yl)-4-(3-phenyl-1-(tetrahydro-2H-pyran-2-yl)-1H-pyrazol-4-yl)quinazoline (Preparation 267, 60 mg, 0.137 mmol) in DCM (2 mL) was placed into a 20-mL sealed tube followed by addition of TFA (0.5 mL). The resulting solution was stirred at rt for 2 h, then concentrated under reduced pressure. The crude product was purified by prep-HPLC conditions: Column: XBridge Prep OBD C18 Column, 30×150 mm 5 um; Mobile Phase A: Water (10 mmol/L NH₄HCO₃+0.1% NH₃·H₂O), Mobile Phase B: MeCN; Flow rate: 60 mL/min; Gradient: 27% B to 37% B in 7 min; to afford the title compound as white solid (17 mg, 35%). LCMS m/z=353 [M+H]⁺. ¹H NMR (300 MHz, DMSO-d₆) δ 9.16 (s, 1H), 8.46 (s, 1H), 8.16 (dd, 3H), 7.90-7.72 (m, 2H), 7.42-7.32 (m, 3H), 7.32-7.22 (m, 3H), 3.90 (s, 3H).

### Example 442:7-(1-(1-methylpiperidin-4-yl)-1H-pyrazol-4-yl)-4-(3-phenyl-1H-pyrazol-4-yl)quinazoline

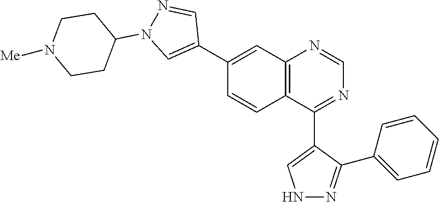 [SMILES: CN1CCC(n2cc(-c3ccc4c(-c5c[nH]nc5-c5ccccc5)ncnc4c3)cn2)CC1]

The title compound was obtained as a yellow solid, 5 mg, 20% yield, from 7-(1-(1-methylpiperidin-4-yl)-1H-pyrazol-4-yl)-4-(3-phenyl-1-(tetrahydro-2H-pyran-2-yl)-1H-pyrazol-4-yl)quinazoline (Preparation 271) following a similar procedure to that described in Example 441. LCMS m/z=436 [M+H]⁺. ¹H NMR (300 MHz, DMSO-d₆) δ 13.59 (s, 1H), 9.17 (s, 1H), 8.57 (s, 1H), 8.19 (d, 3H), 7.82 (d, 2H), 7.52-7.03 (m, 5H), 4.15 (dt, 1H), 2.97-2.77 (m, 2H), 2.22 (s, 3H), 2.13-1.79 (m, 6H).

### Example 443: 4-[3-(2-fluorophenyl)-1H-pyrazol-4-yl]-7-(1-methyl-1H-pyrazol-4-yl)quinazoline

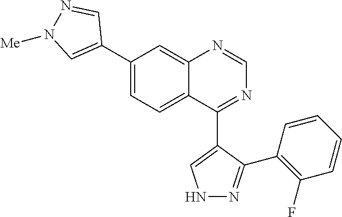 [SMILES: Cn1cc(-c2ccc3c(-c4c[nH]nc4-c4ccccc4F)ncnc3c2)cn1]

The title compound was obtained as a white solid, 10 mg, 25% yield, from 4-(3-(2-fluorophenyl)-1-(tetrahydro-2H-pyran-2-yl)-1H-pyrazol-4-yl)-7-(1-methyl-1H-pyrazol-4-yl)quinazoline (Preparation 268) following a similar procedure to that described in Example 441. LCMS m/z=371 [M+H]⁺. ¹H NMR (300 MHz, DMSO-d₆) δ 13.72 (s, 1H), 9.02 (s, 1H), 8.46 (s, 1H), 8.42-8.24 (m, 1H), 8.22-8.12 (m, 2H), 8.01 (d, 1H), 7.84 (dd, 1H), 7.62 (t, 1H), 7.36 (t, 1H), 7.26 (td, 1H), 7.05 (s, 1H), 3.91 (s, 3H).

### Example 444: 4-(3-(2,5-difluorophenyl)-1H-pyrazol-4-yl)-7-(1-methyl-1H-pyrazol-4-yl)quinazoline

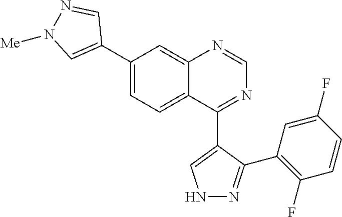 [SMILES: Cn1cc(-c2ccc3c(-c4c[nH]nc4-c4cc(F)ccc4F)ncnc3c2)cn1]

The title compound was obtained as an off-white solid, 20 mg, 31% yield, from 4-(3-(2,5-difluoro phenyl)-1-(tetrahydro-2H-pyran-2-yl)-1H-pyrazol-4-yl)-7-(1-methyl-1H-pyrazol-4-yl)quinazoline (Preparation 269) using a similar procedure to that described in Example 441. LCMS m/z=389 [M+H]⁺. ¹H NMR (300 MHz, DMSO-d₆) δ 13.79 (s, 1H), 9.03 (s, 1H), 8.50 (d, 2H), 8.16 (d, 2H), 8.05 (d, 1H), 7.88 (dd, 1H), 7.48 (s, 1H), 7.14 (d, 2H), 3.91 (s, 3H).

### Examples 445 and 446: (R)-1-(4-(1-(2,2-difluoroethyl)-3-phenyl-1H-pyrazol-4-yl)-7-(1-methyl-1H-pyrazol-4-yl)quinazolin-6-yl)ethan-1-ol and (S)-1-(4-(1-(2,2-difluoroethyl)-3-phenyl-1H-pyrazol-4-yl)-7-(1-methyl-1H-pyrazol-4-yl)quinazolin-6-yl)ethan-1-ol

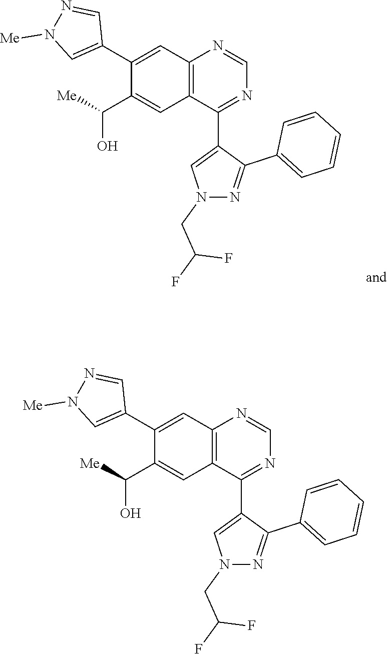 [SMILES: C[C@@H](O)c1cc2c(-c3cn(CC(F)F)nc3-c3ccccc3)ncnc2cc1-c1cnn(C)c1 ; C[C@H](O)c1cc2c(-c3cn(CC(F)F)nc3-c3ccccc3)ncnc2cc1-c1cnn(C)c1]

To a solution of 1-(4-(1-(2,2-difluoroethyl)-3-phenyl-1H-pyrazol-4-yl)-7-(1-methyl-1H-pyrazol-4-yl)quinazolin-6-yl)ethan-1-one (Preparation 279, 220 mg, 0.480 mmol) in MeOH was added NaBH₄ (18.2 mg, 0.479 mmol) at 0° C. The reaction mixture was stirred 0.5 h, then quenched with water and concentrated under reduced pressure, then diluted with water (20 mL) and extracted with EtOAc (2×20 mL). The combined organic layers were washed with brine (15 mL), then dried over Na₂SO₄ and concentrated under reduced pressure. The crude product was purified by prep-HPLC Column: XBridge Shield RP18 OBD Column, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 15% B to 45% B in 8 min: to afford 1-(4-(1-(2,2-difluoroethyl)-3-phenyl-1H-pyrazol-4-yl)-7-(1-methyl-1H-pyrazol-4-yl)quinazolin-6-yl)ethan-1-ol, as a white solid (100 mg, 45%). LCMS m/z=461 [M+H]⁺. This solid (100 mg, 0.217 mmol) was further purified by prep-HPLC Column: Lux 5 μm Cellulose-2, 2.12*25 cm, 5 μm; Mobile Phase A: Hex (0.5% 2M NH₃—MeOH), Mobile Phase B: EtOH: Flow rate: 20 mL/min; Gradient: 30% B isocratic to afford

Peak 1, Example 445, as a yellow solid (32.6 mg), (R)-1-(4-(1-(2,2-difluoroethyl)-3-phenyl-1H-pyrazol-4-yl)-7-(1-methyl-1H-pyrazol-4-yl)quinazolin-6-yl)ethan-1-ol or (S)-1-(4-(1-(2,2-difluoroethyl)-3-phenyl-1H-pyrazol-4-yl)-7-(1-methyl-1H-pyrazol-4-yl)quinazolin-6-yl)ethan-1-ol LCMS m/z=461 [M+H]⁺. ¹H NMR (400 MHZ, MeOH-d₄) δ 9.19 (s, 1H), 8.29 (s, 1H), 8.13 (s, 1H), 7.94 (d, 2H), 7.74 (d, 1H), 7.45-7.35 (m, 2H), 7.31-7.18 (m, 3H), 6.60-6.25 (m, 1H), 5.10 (q, 1H), 4.81 (td, 2H), 4.00 (s, 3H), 1.35-1.25 (m, 1H), 1.08 (d, 3H).

and Peak 2, Example 446, as a yellow solid (26.4 mg), (S)-1-(4-(1-(2,2-difluoroethyl)-3-phenyl-1H-pyrazol-4-yl)-7-(1-methyl-1H-pyrazol-4-yl)quinazolin-6-yl)ethan-1-ol or (R)-1-(4-(1-(2,2-difluoroethyl)-3-phenyl-1H-pyrazol-4-yl)-7-(1-methyl-1H-pyrazol-4-yl)quinazolin-6-yl)ethan-1-ol, LCMS m/z=461 [M+H]⁺. ¹H NMR (400 MHZ, MeOH-d₄) δ 9.19 (s, 1H), 8.29 (s, 1H), 8.13 (s, 1H), 7.94 (d, 2H), 7.74 (d, 1H), 7.45-7.35 (m, 2H), 7.31-7.18 (m, 3H), 6.60-6.25 (m, 1H), 5.10 (q, 1H), 4.81 (td, 2H), 4.00 (s, 3H), 1.35-1.25 (m, 1H), 1.08 (d, 3H).

### Example 447:1-(6-(6-methoxy-4-(1-methyl-3-phenyl-1H-pyrazol-4-yl)quinazolin-7-yl)-2,6-diazaspiro[3.3]heptan-2-yl)ethan-1-one

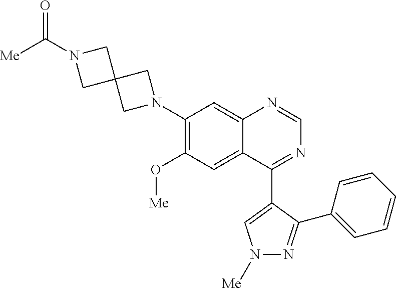 [SMILES: COc1cc2c(-c3cn(C)nc3-c3ccccc3)ncnc2cc1N1CC2(CN(C(C)=O)C2)C1]

XantPhos (11.6 mg, 0.0202 mmol), Pd₂(dba)₃ (9.25 mg, 0.101 mmol) and Cs₂CO₃ (82.1 mg, 0.252 mmol) were added to 7-bromo-6-methoxy-4-(1-methyl-3-phenyl-1H-pyrazol-4-yl)quinazoline (Preparation 281, 40 mg, 0.101 mmol) and 1-(2,6-diazaspiro[3.3]heptan-2-yl)ethan-1-one dihydrochloride (32.1 mg, 0.151 mmol) in dioxane (4 mL) at rt. The reaction mixture was heated to 100° C. for 2 h under N₂. The resulting solution was extracted with EtOAc (3×30 mL). The organic layer was dried with Na₂SO₄ and concentrated under reduced pressure. The crude product was purified by prep-HPLC YMC-Actus Triart C18 ExRS, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 14% B to 44% B in 7 min; to afford the title compound as a yellow solid (16.4 mg, 35%). LCMS m/z=455 [M+H]⁺. ¹H NMR (400 MHZ, DMSO-d₆) δ 8.88 (s, 1H), 8.21 (s, 1H), 7.35-7.19 (m, 5H), 6.72 (s, 1H), 6.53 (s, 1H), 4.26 (d, 6H), 4.01 (d, 5H), 3.39 (s, 3H), 1.75 (s, 3H).

### Example 448: N-(4-(1-methyl-3-phenyl-1H-pyrazol-4-yl)-7-(pyrimidin-4-yl)quinazolin-6-yl)propionamide

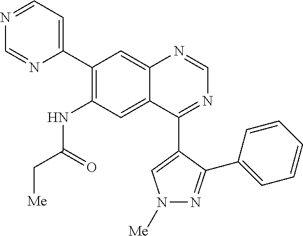 [SMILES: CCC(=O)Nc1cc2c(-c3cn(C)nc3-c3ccccc3)ncnc2cc1-c1ccncn1]

A mixture of 4-(1-methyl-3-phenyl-1H-pyrazol-4-yl)-6-propanamidoquinazolin-7-yl trifluoro methanesulfonate (Preparation 250, 100 mg, 0.197 mmol), 4-(tributylstannyl)pyrimidine (97.1 mg, 0.236 mmol), Pd(PPh₃)₄ (45.4 mg, 0.0394 mmol) and LiCl (16.5 mg, 0.394 mmol) in dioxane was stirred at 100° C. for 2 h, then extracted with EtOAc and water. The organic layer was dried over Na₂SO₄ and concentrated under reduced pressure. The residue was purified by prep-HPLC (Column: YMC-Actus Triart C18 ExRS, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 14% B to 44% B in 7 min;) to yield the title compound as a yellow solid (9.0 mg, 10%). LCMS m/z=436 [M+H]⁺. ¹H NMR (300 MHz, DMSO-d₆) δ 10.90 (s, 1H), 9.38 (d, 1H), 9.21 (s, 1H), 9.01 (d, 1H), 8.70 (s, 1H), 8.44 (s, 1H), 8.31 (s, 1H), 8.13 (dd, 1H), 7.33 (dt, 2H), 7.27-7.19 (m, 3H), 4.06 (s, 3H), 2.31 (q, 2H), 1.04 (t, 3H).

### Example 449: N-(4-(1-methyl-3-phenyl-1H-pyrazol-4-yl)-7-(pyrimidin-2-yl)quinazolin-6-yl)propionamide

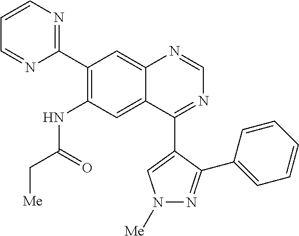 [SMILES: CCC(=O)Nc1cc2c(-c3cn(C)nc3-c3ccccc3)ncnc2cc1-c1ncccn1]

The title compound was obtained as a yellow solid, 3.1 mg, 3.6% yield, from 4-(1-methyl-3-phenyl-1H-pyrazol-4-yl)-6-propionamidoquinazolin-7-yl trifluoromethanesulfonate (Preparation 250) and 2-(tributylstannyl)pyridine, following a similar procedure to that described in Example 448. LCMS m/z=436 [M+H]⁺. ¹H NMR (300 MHz, DMSO-d₆) δ 12.31 (s, 1H), 9.15 (dd, 4H), 9.06 (s, 1H), 8.32 (s, 1H), 7.68 (t, 1H), 7.33 (dd, 2H), 7.26-7.18 (m, 3H), 4.06 (s, 3H), 2.44 (t, 2H), 1.14 (t, 3H).

### Example 450: N-(7-(3-methyl-1H-pyrazol-1-yl)-4-(1-methyl-3-phenyl-1H-pyrazol-4-yl)quinazolin-6-yl)propionamide

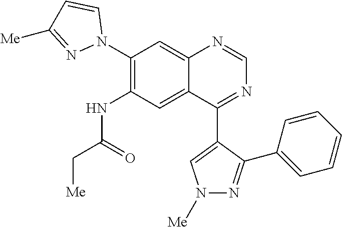 [SMILES: CCC(=O)Nc1cc2c(-c3cn(C)nc3-c3ccccc3)ncnc2cc1-n1ccc(C)n1]

A mixture of 4-(1-methyl-3-phenyl-1H-pyrazol-4-yl)-6-propanamidoquinazolin-7-yl trifluoromethane sulfonate (Preparation 250, 100 mg, 0.197 mmol) in dioxane (5 mL), 3-methyl-1H-pyrazole (32.3 mg, 0.394 mmol), Cs₂CO₃ (128 mg, 0.394 mmol) and E-Phos Pd G4 (72.4 mg, 0.0788 mmol) was stirred at 80° C. for 2 h under N₂. The mixture was diluted with EtOAc (20 mL) and washed with brine (10 mL×2). The organic layer was dried with Na₂SO₄ and concentrated under vacuum. The residue was purified by prep. HPLC (Column: XBridge Shield RP18 OBD Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 21% B to 50% B in 7 min, 50% B; to give the title compound (1.1 mg, yield: 1.26%) as an off-white solid. LCMS m/z=438 [M+H]⁺. ¹H NMR (400 MHz, DMSO-d₆) δ 10.90 (s, 1H), 9.15 (s, 1H), 8.95 (s, 1H), 8.46 (s, 1H), 8.29 (s, 1H), 8.18 (s, 1H), 7.86 (s, 1H), 7.32-7.31 (m, 2H), 7.23-7.22 (m, 3H), 4.05 (s, 3H), 2.37-2.31 (m, 2H), 2.19-2.16 (m, 3H), 1.07 (t, J=7.5 Hz, 3H).

### Example 451: N-(4-(1-methyl-3-phenyl-1H-pyrazol-4-yl)-7-(3-morpholinoprop-1-yn-1-yl)quinazolin-6-yl)propionamide

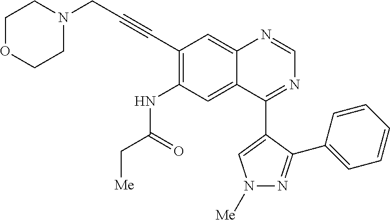 [SMILES: CCC(=O)Nc1cc2c(-c3cn(C)nc3-c3ccccc3)ncnc2cc1C#CCN1CCOCC1]

To a solution of 4-(1-methyl-3-phenyl-1H-pyrazol-4-yl)-6-propionamidoquinazolin-7-yl trifluoro methanesulfonate (Preparation 250, 50 mg, 0.099 mmol), 4-(prop-2-yn-1-yl)morpholine (14.7 mg, 0.118 mmol) and CuI (7.5 mg, 0.197 mmol)) in DMF (2 mL) were added TEA (198 mg, 1.97 mmol) and Pd(PPh₃)₄ (5.70 mg, 0.00494 mmol) at rt under N₂. The reaction mixture was stirred at 100° C. for 2 h. The reaction mixture was extracted with DCM. The solvent was evaporated and the residue purified by Column: XBridge Shield RP18 OBD Column, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 12% B to 51% B in 7 min, 51% B; to afford the title compound as a light yellow solid (7.5 mg, 15%). LCMS m/z=481 [M+H]⁺. ¹H NMR (400 MHZ, DMSO-d₆) δ 9.31 (s, 1H), 9.16 (s, 1H), 8.43 (s, 1H), 8.25 (s, 1H), 8.10 (s, 1H), 7.34-7.08 (m, 5H), 4.04 (s, 3H), 3.70-3.58 (m, 6H), 2.57 (t, 4H), 2.39 (q, 2H), 1.08 (t, 3H).

### Example 452: N-(7-ethyl-4-(1-methyl-3-phenyl-1H-pyrazol-4-yl)pyrido[3,2-d]pyrimidin-6-yl)-3-oxabicyclo[3.1.0]hexane-1-carboxamide

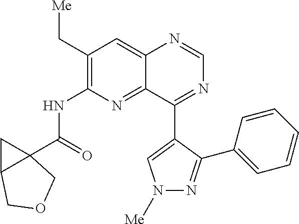 [SMILES: CCc1cc2ncnc(-c3cn(C)nc3-c3ccccc3)c2nc1NC(=O)C12COCC1C2]

EtMgBr (71.5 mg, 0.537 mmol) was added to N-(7-chloro-4-(1-methyl-3-phenyl-1H-pyrazol-4-yl)pyrido[3,2-d]pyrimidin-6-yl)-3-oxabicyclo[3.1.0]hexane-1-carboxamide (Preparation 292, 80 mg, 0.179 mmol) and tris(acetylacetonato) iron (III) (189 mg, 0.537 mmol) in THF/NMP (4 mL/1 mL) at 0° C. The reaction mixture was stirred at rt for 16 h, then diluted with EtOAc (100 mL), washed with water (3×100 mL) and brine (100 mL). The organic layer was dried over Na₂SO₄, filtered and evaporated to dryness. The crude product was purified by prep-TLC (20:1 DCM/MeOH) followed by prep-HPLC (Column: Aeris PEPTIDE 5 μm XB-C18 Axia, 21.2 mm×250 mm, 5 μm; Mobile Phase A: Water (10 mmol/L NH₄HCO₃+0.1% NH₃·H₂O), Mobile Phase B: MeCN; Flow rate: 60 mL/min; Gradient: 28% B to 58% B in 8 min, to afford the title compound as an off-white solid (23.1 mg, 29%). LCMS m/z=451 [M+H]⁺. ¹H NMR (300 MHz, DMSO-d₆) δ 9.88 (s, 1H), 9.00 (s, 1H), 8.93 (s, 1H), 8.22 (s, 1H), 7.49 (dd, 2H), 7.36-7.27 (m, 3H), 4.10-3.93 (m, 5H), 3.80 (s, 2H), 2.82 (q, 2H), 2.39-2.28 (m, 1H), 1.54 (dd, 1H), 1.28 (t, 3H), 0.97 (t, 1H).

### Example 453: N-(4-(3-(2,6-difluorophenyl)-1-methyl-1H-pyrazol-4-yl)-7-(1-methyl-1H-pyrazol-3-yl)quinazolin-6-yl)propionamide

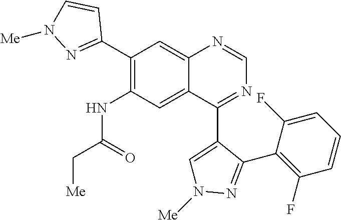 [SMILES: CCC(=O)Nc1cc2c(-c3cn(C)nc3-c3c(F)cccc3F)ncnc2cc1-c1ccn(C)n1]

A mixture of 4-(3-(2,6-difluorophenyl)-1-methyl-1H-pyrazol-4-yl)-7-(1-methyl-1H-pyrazol-3-yl)quinazolin-6-amine (Preparation 285, 25 mg, 0.0598 mmol), T₃PR (0.06 mL, 0.119 mmol), propanoic acid (5.31 mg, 0717 mmol) and pyridine (9.41 mg, 0.119 mmol) in THF (3 mL) was stirred at rt for 45 min, then diluted with EtOAc (50 mL) and washed with brine (2×20 mL). The organic layer was dried with Na₂SO₄ and concentrated under reduced pressure. The residue was purified by prep-HPLC XBridge Shield RP18 OBD Column, 30*150 mm, 5 um; Mobile Phase A: Water (10 mmol/L NH₄HCO₃+0.1% NH₃·H₂O), Mobile Phase B: MeCN: Flow rate: 60 mL/min; Gradient: 15% B to 45% B in 8 min; to yield the title compound as an off-white solid (11.1 mg, 99%). LCMS m/z=474 [M+H]⁺. ¹H NMR (300 MHz, DMSO-d₆) δ 11.75 (s, 1H), 9.41 (s, 1H), 8.91 (s, 1H), 8.54 (s, 1H), 8.42 (s, 1H), 8.01 (d, 1H), 7.62-7.34 (m, 1H), 7.24 (d, 1H), 7.05 (t, 2H), 4.10 (s, 3H), 4.05 (s, 3H), 2.54 (s, 1H), 1.24 (t, 4H).

### Example 454: N-(4-(1-methyl-3-phenyl-1H-pyrazol-4-yl)-7-(3-morpholinoprop-1-yn-1-yl)pyrido[3,2-d]pyrimidin-6-yl)bicyclo[1.1.1]pentane-1-carboxamide

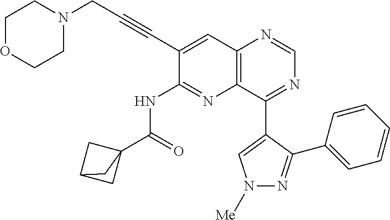 [SMILES: Cn1cc(-c2ncnc3cc(C#CCN4CCOCC4)c(NC(=O)C45CC(C4)C5)nc23)c(-c2ccccc2)n1]

2,4,6-Trichlorobenzoyl chloride (34.4 mg, 0.141 mmol) was added to a mixture of TEA (23.7 mg, 0.235 mmol) and bicyclo[1.1.1]pentane-1-carboxylic acid (15.8 mg, 0.141 mmol) in THF (4 mL) at 0° C. for 1 h. 4-(1-Methyl-3-phenyl-1H-pyrazol-4-yl)-7-(3-morpholinoprop-1-yn-1-yl)pyrido[3,2-d]pyrimidin-6-amine (Preparation 296, 20 mg, 0.0470 mmol) was added and the reaction mixture was stirred at 80° C. for 4 h under N₂. The mixture was diluted with EtOAc (100 mL) and washed with brine (2×50 mL). The organic layer was dried with Na₂SO₄ and concentrated under reduced pressure. The residue was purified by column chromatography (SiO₂, 15:1 DCM/EtOAc) followed by prep-HPLC XBridge Shield RP18 OBD Column, 30*150 mm, 5 um; Mobile Phase A: Water (10 mmol/L NH₄HCO₃+0.1% NH₃·H₂O), Mobile Phase B: MeCN; Flow rate: 60 mL/min; Gradient: 15% B to 45% B in 8 min; to afford the title compound as a yellow solid (1.4 mg, 6%). LCMS m/z=520 [M+H]⁺. ¹H NMR (400 MHZ, DMSO-d₆) δ 9.90 (s, 1H), 9.23 (s, 1H), 8.97 (s, 1H), 8.46 (s, 1H), 7.54-7.47 (m, 2H), 7.33 (dd, 2H), 4.00 (s, 2H), 3.70-3.61 (m, 5H), 2.62-2.51 (m, 7H), 2.18 (s, 6H).

### Example 458: N-(7-methoxy-4-(1-methyl-5-phenyl-1H-pyrazol-4-yl)pyrido[3,2-d]pyrimidin-6-yl)-1-(trifluoromethyl)cyclopropane-1-carboxamide

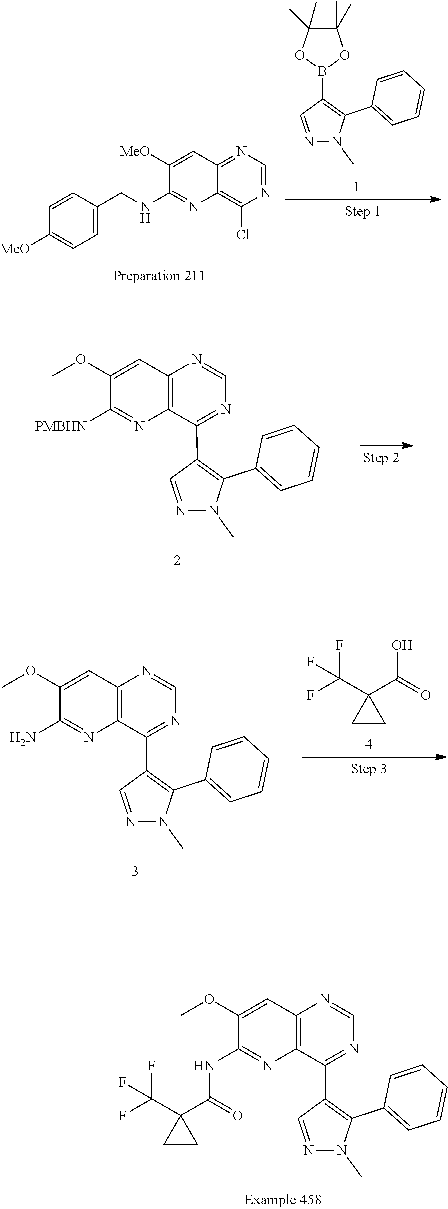

### Step 1: 7-methoxy-N-(4-methoxybenzyl)-4-(1-methyl-5-phenyl-1H-pyrazol-4-yl)pyrido[3,2-d]pyrimidin-6-amine

A mixture of 4-chloro-7-methoxy-N-(4-methoxybenzyl)pyrido[3,2-d]pyrimidin-6-amine (Preparation 211, 1.22 g, 3.7 mmol), 1-methyl-5-phenyl-4-(4,4,5,5-tetramethyl-1,3,2-dioxaborolan-2-yl)-1H-pyrazole (1.16 g, 1.1 eq.) and potassium carbonate (1.03 g, 2.0 eq.) in sulfolane (8.0 mL) and water (4.4 mL) was degassed by bubbling through Nitrogen gas for 10 minutes. [1,1′-Bis(diphenylphosphino)ferrocene]dichloropalladium (II) (268 mg, 10 mol %) was added and the mixture was heated to 90° C. (external temperature). After 1.5 the mixture was cooled down to room temperature and diluted with ethyl acetate (30 mL). The mixture was washed with water (3×20 mL) and the organic layer was dried with sodium sulfate and filtered. The evaporation of the solvent at reduced pressure afforded a red/brown oil. The purification by column chromatography (40 g silica gel, dichloromethane/methanol gradient 0-5%) afforded The title compound as a pale-orange foam (1.45 g, 3.70 mmol, 87% yield). LC-MS: (ES, m/z): RT=1.481 min, LCMS: m/z=453.2 [M+1].

### Step 2: 7-methoxy-4-(1-methyl-5-phenyl-1H-pyrazol-4-yl)pyrido[3,2-d]pyrimidin-6-amine

A mixture of 4-chloro-7-methoxy-N-(4-methoxybenzyl)pyrido[3,2-d]pyrimidin-6-amine (150 mg, 0.33 mmol) and trifluoroacetic acid (2.0 mL) was heated at 90° C. (external temperature) for 1 hr. The reaction mixture was cooled down and concentrated under reduced pressure. The residue was diluted with water (5 mL) and dichloromethane (5 mL) and the pH was adjusted to 9 with saturated aqueous sodium bicarbonate. The layers were separated, and the aqueous layer was extracted with dichloromethane and the combined organic layers were dried with sodium sulfate. Evaporation of the solvent under reduced pressure afforded a brown solid that was recrystallized from dichloromethane and heptanes to afford the title compound (80 mg, 0.24 mmol) as an off-white solid. LC-MS: (ES, m/z): RT=1.34 min, LCMS: m/z=333.2 [M+1].

### Step 3: N-(7-methoxy-4-(1-methyl-5-phenyl-1H-pyrazol-4-yl)pyrido[3,2-d]pyrimidin-6-yl)-1-(trifluoromethyl)cyclopropane-1-carboxamide

7-methoxy-N-(4-methoxybenzyl)-4-(1-methyl-5-phenyl-1H-pyrazol-4-yl)pyrido[3,2-d]pyrimidin-6-amine (80 mg, 0.24 mmol) was placed with 1-(trifluoromethyl)cyclopropane-1-carboxylic acid (56 mg, 1.5 eq.) and chloro-N,N,N′,N′-tetramethylformamidinium hexafluorophosphate (140 mg, 2.0 eq.) in dichloromethane (1.0 mL). N-Methylimidazole (75 μL, 4.0 eq.) was added and the mixture stirred at room temperature overnight. Dichloromethane (5 mL) and water (5 mL) were added, and the layers were separated. The aqueous layer was extracted with dichloromethane (2×5 mL) and the combined organic layers were washed with water. After drying with sodium sulfate and filtration the mixture was concentrated under reduced pressure to afford the title compound as an off-white solid (92 mg, 0.20 mmol). LC-MS: (ES, m/z): RT=1.667 min, LCMS: m/z=469.2 [M+1]. ¹H NMR (400 MHZ, CD₂Cl₂) δ 9.31 (s, 1H): 9.18 (s, 1H): 8.66 (s, 1H): 7.46 (s, 1H): 7.45-7.41 (m, 3H): 7.40-7.31 (m, 2H): 4.09 (s, 3H): 3.72 (s, 3H): 1.70-1.63 (m, 2H); 1.45-1.36 (m, 2H).

### Example 459: (1S)—N-[7-cyclopropoxy-4-(1-methyl-3-phenyl-1H-pyrazol-4-yl)pyrido[3,2-d]pyrimidin-6-yl]-3-oxabicyclo[3.1.0]hexane-1-carboxamide

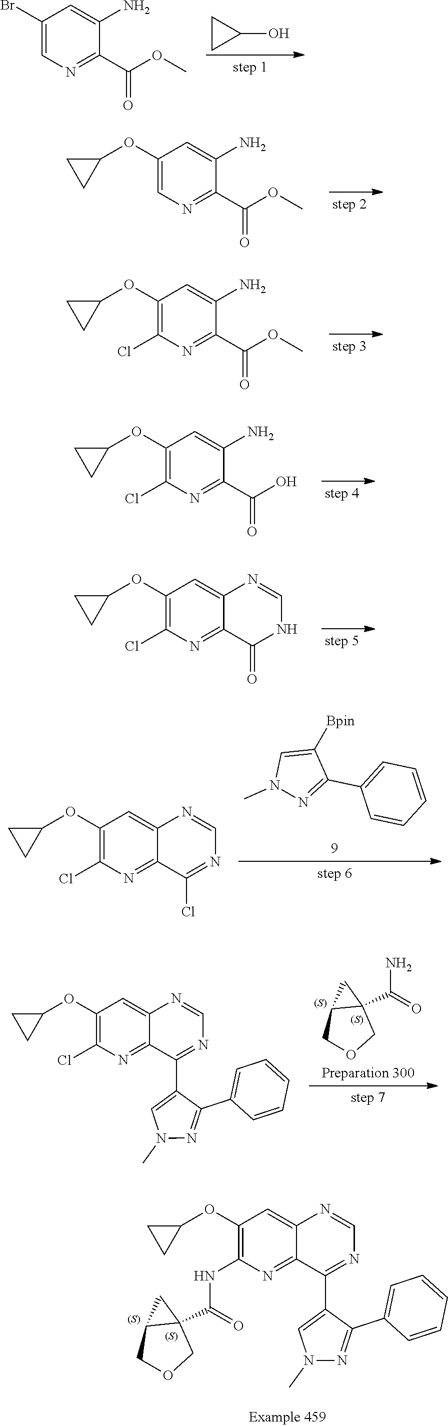

### Step 1: methyl 3-amino-5-cyclopropoxypyridine-2-carboxylate

The reaction mixture of methyl 3-amino-5-bromopyridine-2-carboxylate (4 g, 17.3 mmol), Cs₂CO₃ (8.44 g, 25.9 mmol) and Rock phos Pd(1.41 g, 1.73 mmol), cyclopropanol (1.50 g, 25.9 mmol) was added in dioxane (40 mL) was stirred at 100° C. for 3 h under N₂. The mixture was diluted with EA 200 mL and washed with brine 100 mL*2, the organic layer was dried with Na₂SO₄ and concentrated under vacuum. The residue was purified by a silica gel column with PE:EA=2:1 to afford the title compound (1 g, yield: 22.6%) as an off-white solid. LC-MS: (ES, m/z): RT=0.956 min, LCMS: m/z=209 [M+1].

### Step 2: methyl 3-amino-6-chloro-5-cyclopropoxypyridine-2-carboxylate

NCS (1.01 g, 5.76 mmol) was added to the solution of methyl 3-amino-5-cyclopropoxypyridine-2-carboxylate (1 g, 4.80 mmol) in THF (20 mL) at ice bar. The resulting mixture was stirred at r.t for 2 h. The mixture was diluted with EA 100 mL and washed with brine 50 mL*2, the organic layer was dried with Na₂SO₄ and concentrated under vacuum. The residue was purified by a silica gel column with DCM:EA=15:1. This resulted in the title compound (750 mg, yield: 87.2%) as a light yellow solid. LC-MS: (ES, m/z): RT=0.988 min, LCMS: m/z=243 [M+1].

### Step 3:3-amino-6-chloro-5-cyclopropoxypyridine-2-carboxylic Acid

LiOH (185 mg, 6.18 mmol) was added to methyl 3-amino-6-chloro-5-cyclopropoxypyridine-2-carboxylate (750 mg, 3.09 mmol) to in THF (16 mL) and H₂O (4 mL) at r.t. The resulting mixture was stirred at 40° C. for 10 h. The mixture was adjusted PH=3 with hydrochloric acid. Then diluted with EA 100 mL and washed with brine 50 mL*2, the organic layer was dried with Na₂SO₄ and concentrated under vacuum. The residue was purified by a silica gel column with DCM:MeOH=18:1. This resulted in the title compound (600 mg, 75.2%) as a yellow solid. LC-MS: (ES, m/z): RT=1.112 min, LCMS: m/z=229 [M+1].

### Step 4:6-chloro-7-cyclopropoxy-3H, 4H-pyrido[3,2-d]pyrimidin-4-one

Methanimidamide (230 mg, 5.24 mmol) was added to 3-amino-6-chloro-5-cyclopropoxypyridine-2-carboxylic acid (600 mg, 2.62 mmol) in EtOH (20 mL) at rt. The resulting mixture was stirred at 80° C. for 2 h. The mixture was concentrated under vacuum and then diluted with DCM 100 mL and washed with water 50 mL*2, the organic layer was dried with Na₂SO₄ and concentrated under vacuum. The residue was purified by a silica gel column with DCM:EA=25:1. This resulted in the title compound (300 mg, yield: 52%) as a white solid. LC-MS: (ES, m/z): RT=1.253 min, LCMS: m/z=238 [M+1].

### Step 5: 4,6-dichloro-7-cyclopropoxypyrido[3,2-d]pyrimidine

POCl₃ (593 mg, 3.78 mmol) and N—N-Diethylaniline (945 mg, 6.30 mmol) were added to 6-chloro-7-cyclopropoxy-3H,4H-pyrido[3,2-d]pyrimidin-4-one (300 mg, 1.26 mmol) in CH₃CN (15 mL) at 0° C. The resulting mixture was stirred at 80° C. for 2 h. The mixture was diluted with EA 50 mL and washed with Sodium bicarbonate aqueous solution 50 mL*2, the organic layer was dried with Na₂SO₄ and concentrated under vacuum. The residue was purified by a silica gel column with DCM:EA=25:1. This resulted in the title compound (100 mg, yield: 31%) as a white solid. LC-MS: (ES, m/z): RT=1.323 min, LCMS: m/z=256 [M+1].

### Step 6:4-{6-chloro-7-cyclopropoxypyrido[3,2-d]pyrimidin-4-yl}-1-methyl-3-phenyl-1H-pyrazole

The reaction mixture of Pd(dppf)Cl₂ (28.6 mg, 35.1 μmol), K₃PO₄ (82.2 mg, 386 μmol), 4,6-dichloro-7-cyclopropoxypyrido[3,2-d]pyrimidine (90 mg, 351 μmol) and 1-(5-methyl-1,3,4-oxadiazol-2-yl)ethan-1-ol (99.7 mg, 351 μmol) in dioxane/H₂O (9 mL/3 mL) was heated at 60° C. for 3 h under N₂. The reaction mixture was diluted with EtOAc (60 mL), and washed sequentially with water (30 mL). The organic layer was dried over Na₂SO₄, filtered and concentrated under vacuum. The crude product was purified by Prep-TLC with DCM:EA=1:1. The title compound (93 mg, yield: 70.4%) was collected as a white solid.

LC-MS: (ES, m/z): RT=1.245 min, LCMS: m/z=378 [M+1].

### Step 7: (1S)—N-[7-cyclopropoxy-4-(1-methyl-3-phenyl-1H-pyrazol-4-yl)pyrido[3,2-d]pyrimidin-6-yl]-3-oxabicyclo[3.1.0]hexane-1-carboxamide

The reaction mixture of 4-{6-chloro-7-cyclopropoxypyrido[3,2-d]pyrimidin-4-yl}-1-methyl-3-phenyl-1H-pyrazole (35 mg, 92.6 μmol), (1S,5S)-3-oxabicyclo[3.1.0]hexane-1-carboxamide (Preparation 300, 11.7 mg, 92.6 μmol), XantPhos (10.6 mg, 18.5 μmol), Pd₂(dba)₃ (9.59 mg, 9.26 μmol) and K₂CO₃ (19.0 mg, 138 μmol) in dioxane (5 mL) was heated at 100° C. for 3 hs under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:MeOH=30:1. The residue was purified by Prep-HPLC as following condition: Column: Xselect CSH F-Phenyl OBD column, 19*250 mm, 5 μm: Flow rate: 25 mL/min: Gradient: 55% B to 80% B in 10 min, 80% B: Wave Length: 254/220 nm: RT1 (min): 9.87; The title compound (1.4 mg) was collected as an off-white solid. LC-MS: (ES, m/z): RT=1.199 min, LCMS: m/z=469 [M+1]: 1H NMR (400 MHZ, DMSO-d₆) δ 9.54 (s, 1H), 9.13 (s, 1H), 8.89 (s, 1H), 7.96 (s, 1H), 7.50 (s, 2H), 7.36-7.30 (m, 3H), 4.27 (s, 1H), 4.01 (d, J=8.6 Hz, 5H), 3.78 (s, 2H), 2.32 (s, 1H), 1.52 (s, 1H), 0.96 (s, 3H), 0.85 (s, 2H).

### Example 460: (1S,5S)—N-[7-cyclopropoxy-4-(1-methyl-3-phenyl-1H-pyrazol-4-yl)pyrido[3,2-d]pyrimidin-6-yl]-3-methyl-3-azabicyclo[3.1.0]hexane-1-carboxamide

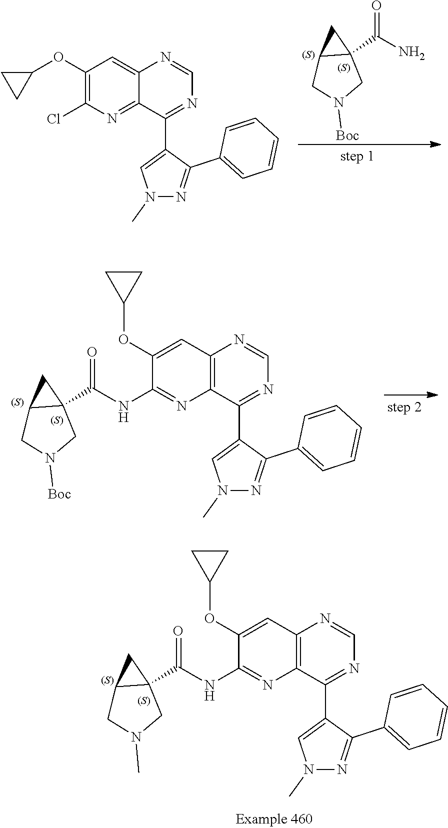

### Step 1: tert-butyl (1S,5S)-1-{[7-cyclopropoxy-4-(1-methyl-3-phenyl-1H-pyrazol-4-yl)pyrido[3,2-d]pyrimidin-6-yl]carbamoyl}-3-azabicyclo[3.1.0]hexane-3-carboxylate

The reaction mixture of XantPhos (30.4 mg, 52.8 μmol), Pd₂(dba)₃ (27.3 mg, 26.4 μmol), K₂CO₃ (54.6 mg, 396 μmol), 4-{6-chloro-7-cyclopropoxypyrido[3,2-d]pyrimidin-4-yl}-1-methyl-3-phenyl-1H-pyrazole (Product of Example 459, Step 6, 100 mg, 264 μmol) and tert-butyl (1S,5S)-1-carbamoyl-3-azabicyclo[3.1.0]hexane-3-carboxylate (Preparation 302, 119 mg, 528 μmol) in dioxane (10 mL) was heated at 100° C. for 3 h under N₂. The reaction mixture was diluted with EtOAc (50 mL), and washed sequentially with water (30 mL). The organic layer was dried over Na₂SO₄, filtered and concentrated under vacuum. The crude product was purified by Prep-TLC with DCM:MeOH=25:1. The title compound (70 mg) was collected as an off-white solid. LC-MS: (ES, m/z): RT=1.106 min, LCMS: m/z=568 [M+1],

### Step 2: (1S,5S)—N-[7-cyclopropoxy-4-(1-methyl-3-phenyl-1H-pyrazol-4-yl)pyrido[3,2-d]pyrimidin-6-yl]-3-methyl-3-azabicyclo[3.1.0]hexane-1-carboxamide

TFA (5 mL) was added to tert-butyl (1S,5S)-1-{[7-cyclopropoxy-4-(1-methyl-3-phenyl-1H-pyrazol-4-yl)pyrido[3,2-d]pyrimidin-6-yl]carbamoyl}-3-azabicyclo[3.1.0]hexane-3-carboxylate (66 mg, 116 μmol) in DCM (15 mL) at rt. The resulting mixture was stirred at rt for 1 h. The mixture was concentrated vacuum to afford 60 mg (1S,5S)—N-[7-cyclopropoxy-4-(1-methyl-3-phenyl-1H-pyrazol-4-yl)pyrido[3,2-d]pyrimidin-6-yl]-3-azabicyclo[3.1.0]hexane-1-carboxamide as a white solid. NaCNBH₃ (14.6 mg, 232 μmol) was added batchwise to (1S,5S)—N-[7-cyclopropoxy-4-(1-methyl-3-phenyl-1H-pyrazol-4-yl)pyrido[3,2-d]pyrimidin-6-yl]-3-azabicyclo[3.1.0]hexane-1-carboxamide (60 mg, 116 μmol) and HCHO (1 mL) in MeOH/CH COOH (10 mL/1 mL) at 0° C. The resulting mixture was stirred at rt for 3 h. The reaction mixture was diluted with EtOAc (30 mL), and washed sequentially with water (20 mL). The organic layer was dried over Na₂SO₄, filtered and concentrated under vacuum. The crude product was purified by Prep-TLC with DCM:MeOH=20:1. The residue was purified by Prep-HPLC as following condition: Column: Sunfire prep C18 column, 30*150 mm, 5 μm; Mobile Phase A: Water (0.1% FA), Mobile Phase B: ACN: Flow rate: 60 mL/min; Gradient: 5% B to 28% B in 7 min: Wave Length: 254/220 nm to afford the title compound (15.0 mg) as a white solid. LC-MS: (ES, m/z): RT=1.717 min, LCMS: m/z=482 [M+1]: 1H NMR (400 MHZ, DMSO-d₆) δ 9.30 (s, 1H), 9.21 (s, 1H), 8.86 (s, 1H), 7.93 (s, 1H), 7.55-7.47 (m, 2H), 7.33 (dd, J=5.1, 2.0 Hz, 3H), 4.27 (dt, J=6.0, 3.1 Hz, 1H), 3.98 (s, 3H), 3.21 (d, J=8.5 Hz, 1H), 2.94 (d, J=8.9 Hz, 1H), 2.65 (d, J=8.6 Hz, 1H), 2.38 (dd, J=8.9, 3.6 Hz, 1H), 2.29 (s, 3H), 2.08 (dq, J=8.3, 4.3, 3.9 Hz, 1H), 1.39 (t, J=4.4 Hz, 1H), 1.28 (dd, J=8.3, 3.8 Hz, 1H), 0.95 (dd, J=5.6, 2.9 Hz, 2H), 0.95-0.82 (m, 2H).

### Example 461: N-{7-cyclopropoxy-4-[3-(4-fluorophenyl)-1-methyl-1H-pyrazol-4-yl]pyrido[3,2-d]pyrimidin-6-yl}-3-(dimethylamino) bicyclo[1.1.1]pentane-1-carboxamide

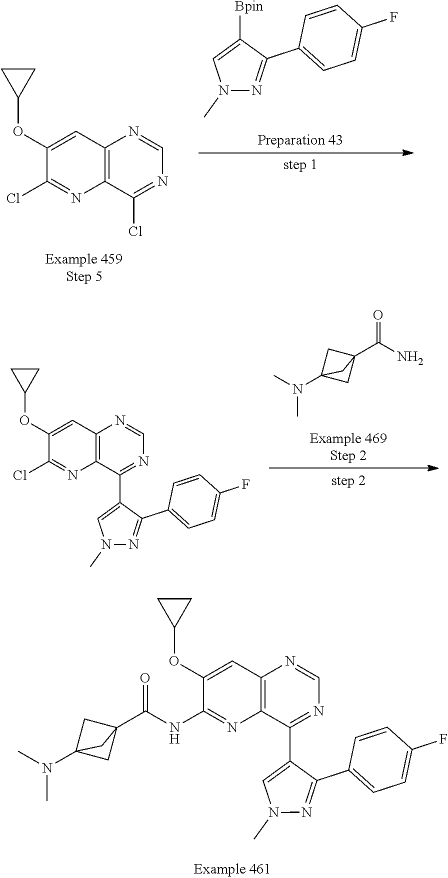

### Step 1: 4-{6-chloro-7-cyclopropoxypyrido[3,2-d]pyrimidin-4-yl}-3-(4-fluorophenyl)-1-methyl-1H-pyrazole

The reaction mixture of Pd(dppf)Cl₂ (38.1 mg, 46.8 μmol), K₃PO₄ (104 mg, 491 μmol), 4,6-dichloro-7-cyclopropoxypyrido[3,2-d]pyrimidine (Product of Example 459, Step 5, 120 mg, 468 μmol) and 3-(4-fluorophenyl)-1-methyl-4-(4,4,5,5-tetramethyl-1,3,2-dioxaborolan-2-yl)-1H-pyrazole (Preparation 43, 127 mg, 421 μmol) in dioxane/H₂O (9 mL/3 mL) was heated at 60° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:EA=15:1. The title compound (66 mg) was collected as an off-white solid. LC-MS: (ES, m/z): RT=1.070 min, LCMS: m/z=396 [M+1].

### Step 2: N-{7-cyclopropoxy-4-[3-(4-fluorophenyl)-1-methyl-1H-pyrazol-4-yl]pyrido[3,2-d]pyrimidin-6-yl}-3-(dimethylamino) bicyclo[1.1.1]pentane-1-carboxamide

The reaction mixture of 4-{6-chloro-7-cyclopropoxypyrido[3,2-d]pyrimidin-4-yl}-3-(4-fluorophenyl)-1-methyl-1H-pyrazole (55 mg, 138 μmol), 3-(dimethylamino) bicyclo[1.1.1]pentane-1-carboxamide (Product of Example 469: Step 2, 42.5 mg, 276 μmol), XantPhos (15.8 mg, 27.6 μmol), Pd₂(dba)₃ (14.2 mg, 13.8 μmol) and Cs₂CO₃ (67.4 mg, 207 μmol) in toluene (5 mL) at rt. The resulting mixture was heated at 80° C. for 3 h under N₂. The reaction mixture was diluted with EtOAc (50 mL), and washed sequentially with water (30 mL). The organic layer was dried over Na₂SO₄, filtered and concentrated under vacuum. The crude product was purified by Prep-TLC with DCM:MeOH=25:1. The residue was purified by Prep-HPLC as following condition: Column: XBridge Prep OBD C18 Column, 30*150 mm, 5 μm; Mobile Phase A: Water (10 mmol/L NH₂HCO₃), Mobile Phase B: ACN: Flow rate: 60 mL/min; Gradient: 27% B to 57% B in 7 min, 57% B: Wave Length: 220 nm: RT1 (min): 5.22: The title compound (7.1 mg) was collected as a white solid.

LC-MS: (ES, m/z): RT=5.422 min, LCMS: m/z=514 [M+1]: 1H NMR (400 MHZ, DMSO-d₆) δ 9.48 (s, 1H), 9.38 (s, 1H), 8.87 (s, 1H), 7.94 (s, 1H), 7.61-7.52 (m, 2H), 7.16 (t, J=8.9 Hz, 2H), 4.36 (s, 1H), 3.99 (s, 3H), 2.17 (s, 6H), 2.08 (s, 6H), 0.96 (d, J=5.7 Hz, 2H), 0.87 (s, 2H).

### Example 462—Intentionally Omitted

### Example 463: N-[7-cyclopropoxy-4-(1-methyl-3-phenyl-1H-pyrazol-4-yl)pyrido[3,2-d]pyrimidin-6-yl]-1-(trifluoromethyl)cyclopropane-1-carboxamide

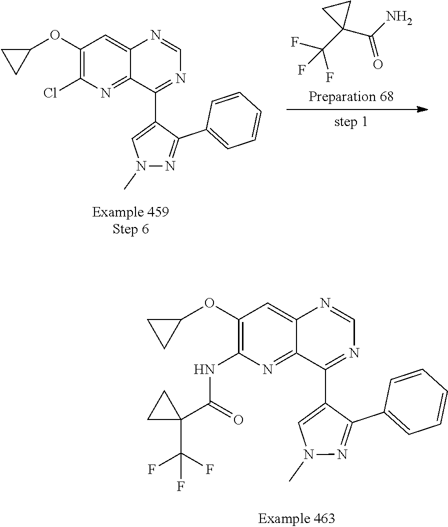 [SMILES: Cn1cc(-c2ncnc3cc(OC4CC4)c(Cl)nc23)c(-c2ccccc2)n1 ; NC(=O)C1(C(F)(F)F)CC1 ; Cn1cc(-c2ncnc3cc(OC4CC4)c(NC(=O)C4(C(F)(F)F)CC4)nc23)c(-c2ccccc2)n1]

### Step 1: N-[7-cyclopropoxy-4-(1-methyl-3-phenyl-1H-pyrazol-4-yl)pyrido[3,2-d]pyrimidin-6-yl]-1-(trifluoromethyl)cyclopropane-1-carboxamide

The reaction mixture of 4-{6-chloro-7-cyclopropoxypyrido[3,2-d]pyrimidin-4-yl}-1-methyl-3-phenyl-1H-pyrazole (Product of Example 459, step 6, 45 mg, 119 μmol), 1-(trifluoromethyl)cyclopropane-1-carboxamide (Preparation 68, 36.4 mg, 238 μmol), XantPhos (13.7 mg, 23.8 μmol), Pd₂(dba)₃ (12.3 mg, 11.9 μmol) and K₂CO₃ (24.5 mg, 178 μmol) in dioxane (5 mL) was heated at 100° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:MeOH=25:1. The residue was purified by Prep-HPLC as following condition: Column: XBridge Prep OBD C18 Column, 30*150 mm, 5 um; Mobile Phase A: Water (10 mmol/L NH₂HCO₃+0.1% NH₃·H₂O), Mobile Phase B: ACN; Flow rate: 60 mL/min; Gradient: 42% B to 67% B in 8 min, 67% B; Wave Length: 254/220 nm. The title compound (22.2 mg) was collected as a yellow solid. LC-MS: (ES, m/z): RT=1.244 min, LCMS: m/z=495 [M+1]: 1H NMR (400 MHZ, DMSO-d₆) δ 9.79 (s, 1H), 9.07 (s, 1H), 8.91 (s, 1H), 7.97 (s, 1H), 7.52-7.45 (m, 2H), 7.32 (dd, J=5.1, 2.0 Hz, 3H), 4.30 (dt, J=5.9, 3.1 Hz, 1H), 3.99 (s, 3H), 1.59 (s, 2H), 1.56-1.41 (m, 2H), 1.23 (s, 1H), 1.00-0.91 (m, 2H), 0.81 (q, J=3.8, 2.5 Hz, 2H).

### Example 464: N-[7-hydroxy-4-(1-methyl-3-phenyl-1H-pyrazol-4-yl)pyrido[3,2-d]pyrimidin-6-yl]-1-(trifluoromethyl)cyclopropane-1-carboxamide

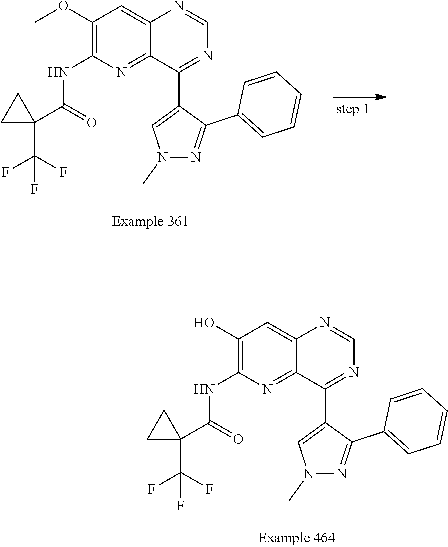 [SMILES: COc1cc2ncnc(-c3cn(C)nc3-c3ccccc3)c2nc1NC(=O)C1(C(F)(F)F)CC1 ; Cn1cc(-c2ncnc3cc(O)c(NC(=O)C4(C(F)(F)F)CC4)nc23)c(-c2ccccc2)n1]

L-Selectride (218 mg, 852 μmol) was added to N-[7-methoxy-4-(1-methyl-3-phenyl-1H-pyrazol-4-yl)pyrido[3,2-d]pyrimidin-6-yl]-1-(trifluoromethyl)cyclopropane-1-carboxamide (Example 361, 200 mg, 426 μmol) in THF (10 mL) at ice bar. The mixture was stirred at 80° C. for 2 h. The mixture was diluted with EA 100 mL and washed with brine 50 mL*2, the organic layer was dried with Na₂SO₄ and concentrated under vacuum. The residue was purified by a Prep-TLC with DCM:MeOH=25:1. The residue was purified by Column: XBridge Shield RP18 OBD Column, 30*150 mm, 5 um; Mobile Phase A: Water (10 mmol/L NH₄HCO₃), Mobile Phase B: ACN; Flow rate: 60 mL/min; Gradient: 9% B to 34% B in 8 min: Wave Length: 220/254 nm. The resulted in the tittle compound (156.5 mg, yield: 80%) as a yellow solid. LC-MS: (ES, m/z): RT=0.737 min, LCMS: m/z=455 [M+1]: 1H NMR (400 MHZ, DMSO-d₆) δ 9.81 (s, 1H), 9.33 (s, 1H), 8.71 (s, 1H), 7.50 (dd, J=6.8, 2.8 Hz, 2H), 7.32 (dd, J=5.2, 2.0 Hz, 4H), 3.98 (s, 3H), 1.62 (s, 2H), 1.50-1.42 (m, 2H).

### Example 465: N-(7-(difluoromethoxy)-4-(1-methyl-3-phenyl-1H-pyrazol-4-yl)pyrido[3,2-d]pyrimidin-6-yl)-1-(trifluoromethyl)cyclopropane-1-carboxamide

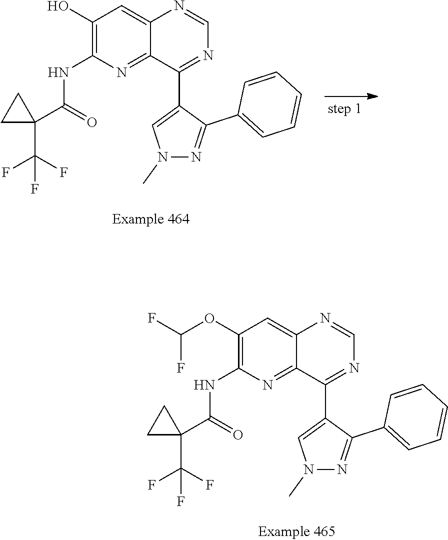 [SMILES: Cn1cc(-c2ncnc3cc(O)c(NC(=O)C4(C(F)(F)F)CC4)nc23)c(-c2ccccc2)n1 ; Cn1cc(-c2ncnc3cc(OC(F)F)c(NC(=O)C4(C(F)(F)F)CC4)nc23)c(-c2ccccc2)n1]

### Step 1: N-(7-(difluoromethoxy)-4-(1-methyl-3-phenyl-1H-pyrazol-4-yl)pyrido[3,2-d]pyrimidin-6-yl)-1-(trifluoromethyl)cyclopropane-1-carboxamide

KF (64.3 mg, 1.09 mmol) and KI (182 mg, 1.09 μmol) were added to N-[7-hydroxy-4-(1-methyl-3-phenyl-1H-pyrazol-4-yl)pyrido[3,2-d]pyrimidin-6-yl]-1-(trifluoromethyl)cyclopropane-1-carboxamide (Example 464, 100 mg, 220 μmol) and diethyl (bromodifluoromethyl)phosphonate (291 mg, 1.09 mmol) in ACN (5 mL) at 0° C. The reaction mixture was stirred at rt for 4 h. The resulting solution was extracted with 3×50 mL of EA. The organic layer was dried with Na₂SO₄ and concentrated under vacuum. The crude product was purified by Prep-HPLC as following condition: Column: XBridge Shield RP18 OBD Column, 30*150 mm, 5 um; Mobile Phase A: Water (10 mmol/L NH₄HCO₃+0.1% NH₃·H₂O), Mobile Phase B: ACN; Flow rate: 60 mL/min; Gradient: 35% B to 68% B in 7 min: Wave Length: 254/220 nm; This resulted in the title compound (15.8 mg, yield: 14%) as a light yellow solid. LC-MS: (ES, m/z): RT=1.342 min, LCMS: m/z=505 [M+1]: 1H NMR (300 MHz, DMSO-d₆) δ 10.25 (s, 1H), 9.00 (d, J=10.1 Hz, 2H), 8.08 (s, 1H), 7.67 (d, J=72.3 Hz, 1H), 7.49 (s, 2H), 7.31 (p, J=3.5 Hz, 3H), 4.01 (s, 3H), 1.61 (s, 2H), 1.48 (t, J=3.8 Hz, 2H).

### Example 466: N-[7-(difluoromethoxy)-4-[3-(4-fluorophenyl)-1-methyl-1H-pyrazol-4-yl]pyrido[3,2-d]pyrimidin-6-yl]-3-(dimethylamino) bicyclo[1.1.1]pentane-1-carboxamide

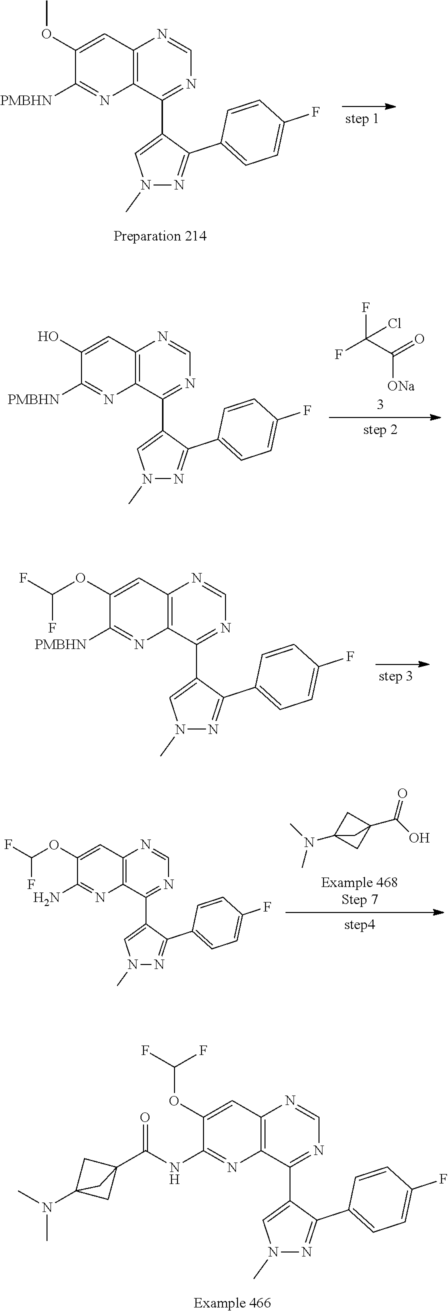

### Step 1:4-[3-(4-fluorophenyl)-1-methyl-1H-pyrazol-4-yl]-6-{[(4-methoxyphenyl)methyl]amino}pyrido[3,2-d]pyrimidin-7-ol

L-Selectride (817 mg, 3.18 mmol) was added to 4-[3-(4-fluorophenyl)-1-methyl-1H-pyrazol-4-yl]-7-methoxy-N-[(4-methoxyphenyl)methyl]pyrido[3,2-d]pyrimidin-6-amine (Preparation 214, 1.0 g, 2.12 mmol) in THF (20 mL) at r.t. The resulting mixture was stirred at 80° C. for 2 h. The mixture was diluted with EA (100 mL) and washed with brine (50 mL*2), the organic layer was dried with Na₂SO₄ and concentrated under vacuum. The residue was purified by a silica gel column with DCM:EA=25:1. This resulted in the title compound (550 mg, yield: 53.4%) as a white solid. A. LC-MS: (ES, m/z): RT=0.945 min, LCMS: m/z=457 [M+1].

### Step 2: Synthesis of 7-(difluoromethoxy)-4-[3-(4-fluorophenyl)-1-methyl-1H-pyrazol-4-yl]-N-[(4-methoxyphenyl)methyl]pyrido[3,2-d]pyrimidin-6-amine

sodium 2-chloro-2,2-difluoroacetate (332 mg, 2.18 mmol) was added to the mixture of 4-[3-(4-fluorophenyl)-1-methyl-1H-pyrazol-4-yl]-6-{[(4-methoxyphenyl)methyl]amino}pyrido[3,2-d]pyrimidin-7-ol (500 mg, 1.09 mmol) and K₂CO₃ (224 mg, 1.63 mmol) in DMF (16 mL) and H₂O (4 mL) at r.t. The resulting mixture was stirred at 80° C. for 3 h under N₂. The mixture was diluted with EA 50 mL and washed with brine 20 mL*2, the organic layer was dried with Na₂SO₄ and concentrated under vacuum. The residue was purified by a silica gel column with DCM:MeOH=18:1. This resulted in the title compound (156 mg, yield: 31.4%) as a white solid. LC-MS: (ES, m/z): RT=1.341 min, LCMS: m/z=507 [M+1].

### Step 3: 7-(difluoromethoxy)-4-[3-(4-fluorophenyl)-1-methyl-1H-pyrazol-4-yl]pyrido[3,2-d]pyrimidin-6-amine

The reaction mixture of 7-(difluoromethoxy)-4-[3-(4-fluorophenyl)-1-methyl-1H-pyrazol-4-yl]-N-[(4-methoxyphenyl)methyl]pyrido[3,2-d]pyrimidin-6-amine (156 mg, 308 μmol) in TFA (15 mL) was stirred at 60° C. for 3 h. The mixture was concentrated under vacuum and then diluted with DCM 50 mL and washed with sodium bicarbonate aqueous solution 50 mL*2, the organic layer was dried with Na₂SO₄ and concentrated under vacuum. The residue was purified by a silica gel column with DCM:MeOH=18:1. This resulted in the title compound (70 mg, 33.8%) as a white solid. LC-MS: (ES, m/z): RT=1.251 min, LCMS: m/z=387 [M+1].

### Step 4: N-[7-(difluoromethoxy)-4-[3-(4-fluorophenyl)-1-methyl-1H-pyrazol-4-yl]pyrido[3,2-d]pyrimidin-6-yl]-3-(dimethylamino) bicyclo[1.1.1]pentane-1-carboxamide

The mixture of Py (2 mL) and T₃P (2 mL), 3-(dimethylamino) bicyclo[1.1.1]pentane-1-carboxylic acid (Product of Example 468 Step 7, 60.0 mg, 387 μmol), and 7-(difluoromethoxy)-4-[3-(4-fluorophenyl)-1-methyl-1H-pyrazol-4-yl]pyrido[3,2-d]pyrimidin-6-amine (50 mg, 129 μmol) in THF (3 mL) was stirred at 50° C. for 3 h. The mixture was concentrated under vacuum. The residue was purified by a Prep-TLC with DCM:EA=18:1. The residue was purified by prep-HPLC as following condition: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 29% B to 54% B in 8 min: Wave Length: 220/254 nm: This resulted in the title compound (9.0 mg) as an off-white solid. LC-MS: (ES, m/z): RT=1.381 min, LCMS: m/z=524 [M+1]: 1H NMR (400 MHZ, DMSO-d₆) δ 10.05 (s, 1H), 9.19 (s, 1H), 8.98 (s, 1H), 8.05 (d, J=1.1 Hz, 1H), 7.60-7.50 (m, 3H), 7.20-7.10 (m, 2H), 4.00 (s, 3H), 2.16 (s, 6H), 2.07 (s, 6H).

### Example 467: (1S,5S)—N-{4-[3-(4-fluorophenyl)-1H-pyrazol-4-yl]-7-methoxypyrido[3,2-d]pyrimidin-6-yl}-3-oxabicyclo[3.1.0]hexane-1-carboxamide

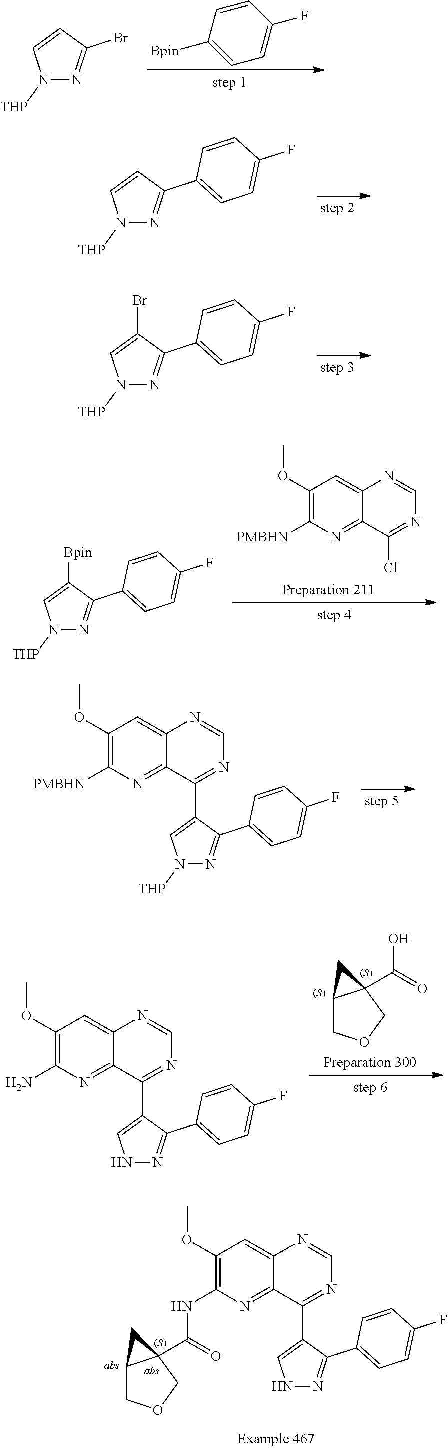

### Step 1: 3-(4-fluorophenyl)-1-(oxan-2-yl)-1H-pyrazole

The reaction mixture of Pd(pddf)Cl₂ (705 mg, 865 μmol), K₂CO₃ (1.78 g, 12.9 mmol), 3-bromo-1-(oxan-2-yl)-1H-pyrazole (2 g, 8.65 mmol) and 2-(4-fluorophenyl)-4,4,5,5-tetramethyl-1,3,2-dioxaborolane (3.84 g, 17.3 mmol) in dioxane/H₂O (20 mL/5 mL) was heated at 100° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by a silica gel column with PE:EA=3:1. The title compound (1.9 g) was collected as a yellow oil. LC-MS: (ES, m/z): RT=1.074 min, LCMS: m/z=247 [M+1].

### Step 2: 4-bromo-3-(4-fluorophenyl)-1-(oxan-2-yl)-1H-pyrazole

NBS (984 mg, 5.53 mmol) was added batchwise to 3-(4-fluorophenyl)-1-(oxan-2-yl)-1H-pyrazole (1.05 g, 4.26 mmol) in DMF (10 mL) at rt. The resulting mixture was stirred at room temperature for 16 h under N₂. The reaction mixture was diluted with EtOAc (100 mL), and washed sequentially with water (50 mL). The organic layer was dried over Na₂SO₄, filtered and concentrated under vacuum. The crude product was purified by a silica gel column with PE:EA=4:1. The title compound (846 mg) was collected as a yellow oil. LC-MS: (ES, m/z): RT=1.380 min, LCMS: m/z=325 [M+1].

### Step 3:3-(4-fluorophenyl)-1-(oxan-2-yl)-4-(4,4,5,5-tetramethyl-1,3,2-dioxaborolan-2-yl)-1H-pyrazole

The reaction mixture of Pd(pddf)Cl₂ (200 mg, 246 μmol), AcOK (364 mg, 3.68 mmol), 4-bromo-3-(4-fluorophenyl)-1-(oxan-2-yl)-1H-pyrazole (800 mg, 2.46 mmol) and 4,4,5,5-tetramethyl-2-(4,4,5,5-tetramethyl-1,3,2-dioxaborolan-2-yl)-1,3,2-dioxaborolane (1.24 g, 4.92 mmol) in DMSO (10 mL) was heated at 60° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by a silica gel column with PE:EA=3:1. The title compound (1 g) was collected as a white solid. LC-MS: (ES, m/z): RT=1.249 min, LCMS: m/z=373 [M+1].

### Step 4: 4-[3-(4-fluorophenyl)-1-(oxan-2-yl)-1H-pyrazol-4-yl]-7-methoxy-N-[(4-methoxyphenyl)methyl]pyrido[3,2-d]pyrimidin-6-amine

The reaction mixture of Pd(pddf)Cl₂ (123 mg, 151 μmol), K₂CO₃ (311 mg, 2.26 mmol), 4-chloro-7-methoxy-N-[(4-methoxyphenyl)methyl]pyrido[3,2-d]pyrimidin-6-amine (Preparation 211, 500 mg, 1.51 mmol) and 3-(4-fluorophenyl)-1-(oxan-2-yl)-4-(4,4,5,5-tetramethyl-1,3,2-dioxaborolan-2-yl)-1H-pyrazole (562 mg, 1.51 mmol) in dioxane/H₂O (8 mL/2 mL) was heated at 80° C. for 3 h under N₂. The reaction mixture was diluted with EtOAc (60 mL), and washed sequentially with water (40 mL). The organic layer was dried over Na₂SO₄, filtered and concentrated under vacuum. The crude product was purified by Prep-TLC with DCM:MeOH=25:1. The title compound (428 mg) was collected as a white solid. LC-MS: (ES, m/z): RT=1.099 min, LCMS: m/z=541 [M+1].

### Step 5: 4-[3-(4-fluorophenyl)-1H-pyrazol-4-yl]-7-methoxypyrido[3,2-d]pyrimidin-6-amine

The reaction mixture of 4-[3-(4-fluorophenyl)-1-(oxan-2-yl)-1H-pyrazol-4-yl]-7-methoxy-N-[(4-methoxyphenyl)methyl]pyrido[3,2-d]pyrimidin-6-amine (420 mg, 776 μmol) in TFA (10 mL) was heated at 60° C. for 3 h. The reaction mixture was concentrated under vacuum and then diluted with EtOAc (30 mL), and washed sequentially with (sat) NaHCO₃(aq.) (20 mL). The organic layer was dried over Na₂SO₄, filtered and concentrated under vacuum. The title compound (348 mg) was collected as a white solid. LC-MS: (ES, m/z): RT=0.617 min, LCMS: m/z=337 [M+1].

### Step 6: (1S,5S)—N-{4-[3-(4-fluorophenyl)-1H-pyrazol-4-yl]-7-methoxypyrido[3,2-d]pyrimidin-6-yl}-3-oxabicyclo[3.1.0]hexane-1-carboxamide

2,4,6-trichlorobenzoyl chloride (331 mg, 1.36 mmol) was added to (1S,5S)-3-oxabicyclo[3.1.0]hexane-1-carboxylic acid (Preparation 300, 174 mg, 1.36 mmol) and TEA (696 mg, 6.83 mmol) in THF (10 mL) at 0° C. The resulting solution was stirred at rt for 30 min. And 4-[3-(4-fluorophenyl)-1H-pyrazol-4-yl]-7-methoxypyrido[3,2-d]pyrimidin-6-amine (230 mg, 683 μmol) was added to the mixture at rt. The resulting solution was stirred at 80° C. for 16 h. The reaction mixture was diluted with EtOAc (60 mL), and washed sequentially with water (30 mL). The organic layer was dried over Na₂SO₄, filtered and concentrated under vacuum. The crude product was purified by Prep-TLC with DCM:MeOH=20:1. The residue was purified by Prep-HPLC as following condition: Column: YMC-Actus Triart C18 ExRS, 30*150 mm, 5 um; Mobile Phase A: Water (10 mmol/L NH₄HCO₃+0.1% NH₃·H₂O), Mobile Phase B: ACN: Flow rate: 60 mL/min; Gradient: 34% B to 60% B in 10 min, 60% B: Wave Length: 254; 220 nm; RT1 (min): 4.85/9.18: The title compound (42.3 mg) was collected as an off-white solid. LC-MS: (ES, m/z): RT=1.100 min, LCMS: m/z=421 [M+]; ¹H NMR (300 MHz, DMSO-d₆) δ 13.49 (s, 1H), 9.49 (s, 1H), 9.32 (s, 1H), 8.87 (s, 1H), 7.73 (s, 1H), 7.57 (dd, J=8.7, 5.6 Hz, 2H), 7.19 (s, 2H), 4.14-3.96 (m, 5H), 3.79 (d, J=1.6 Hz, 2H), 3.18 (d, J=5.2 Hz, 1H), 2.32 (t, J=7.0 Hz, 1H), 1.52 (dd, J=8.3, 4.5 Hz, 1H), 1.24 (s, 1H), 0.97 (t, J=4.9 Hz, 1H).

### Example 468: N-[7-(difluoromethoxy)-4-[3-(2-fluorophenyl)-1-methyl-1H-pyrazol-4-yl]pyrido[3,2-d]pyrimidin-6-yl]-3-(dimethylamino) bicyclo[1.1.1]pentane-1-carboxamide

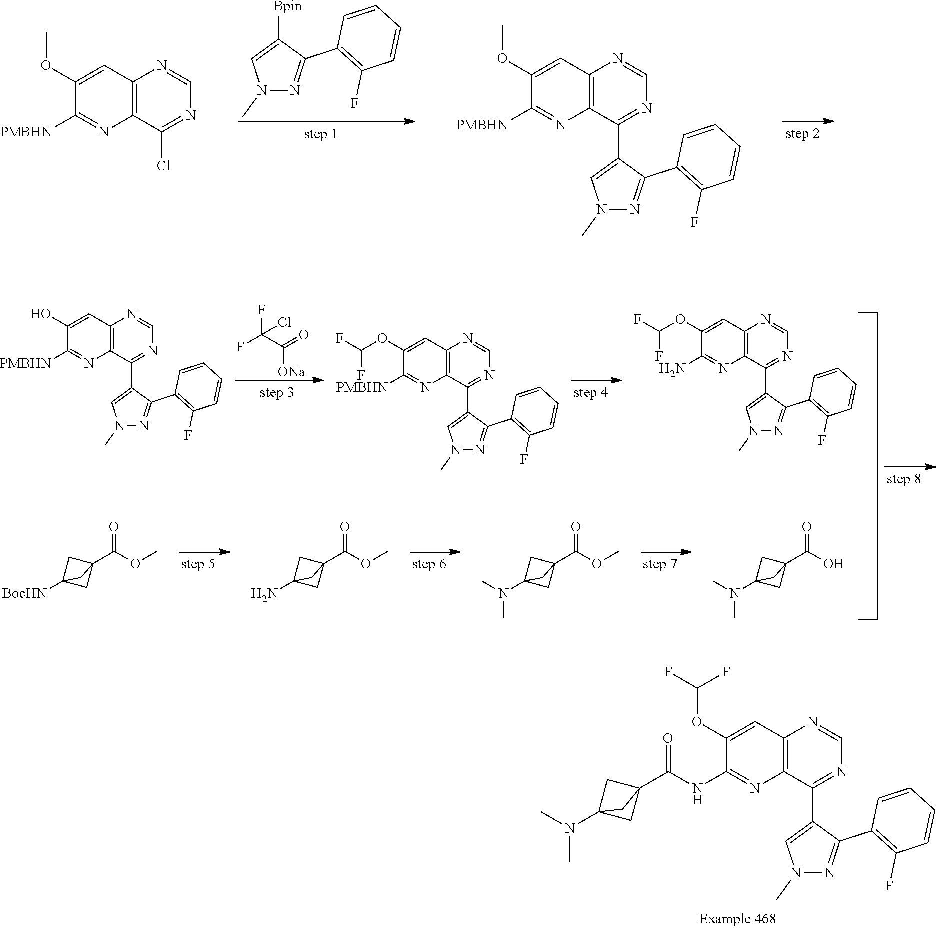

### Step 1: 4-[3-(2-fluorophenyl)-1-methyl-1H-pyrazol-4-yl]-7-methoxy-N-[(4-methoxyphenyl)methyl]pyrido[3,2-d]pyrimidin-6-amine

The reaction mixture of Pd(dppf)Cl₂ (246 mg, 302 μmol), K₂CO₃ (625 mg, 4.53 mmol), 4-chloro-7-methoxy-N-[(4-methoxyphenyl)methyl]pyrido[3,2-d]pyrimidin-6-amine (Preparation 211, 1 g. 3.02 mmol) and 3-(2-fluorophenyl)-1-methyl-4-(4,4,5,5-tetramethyl-1,3,2-dioxaborolan-2-yl)-1H-pyrazole (Preparation 37, 1.09 g, 3.62 mmol) in H₂O (9 mL) and dioxane (36 mL) was stirred at 80° C. for 2 h under N₂. The mixture was diluted with DCM 60 mL and washed with water 30 mL*2, the organic layer was dried with Na₂SO₄ and concentrated under vacuum. The residue was purified by a silica gel column with DCM:MeOH=20:1. This resulted in the title compound (800 mg, yield: 87%) as a white solid. LC-MS: (ES, m/z): RT=1.109 min, LCMS: m/z=471 [M+1].

### Step 2: 4-[3-(2-fluorophenyl)-1-methyl-1H-pyrazol-4-yl]-6-{[(4-methoxyphenyl)methyl]amino}pyrido[3,2-d]pyrimidin-7-ol

L-Selectride (686 mg, 2.67 mmol) was added to 4-[3-(2-fluorophenyl)-1-methyl-1H-pyrazol-4-yl]-7-methoxy-N-[(4-methoxyphenyl)methyl]pyrido[3,2-d]pyrimidin-6-amine (840 mg, 1.78 mmol) in THF (20 mL) at r.t. The resulting mixture was stirred at 80° C. for 2 h. The mixture was concentrated under vacuum. The residue was purified by a silica gel column with DCM:EA=25:1. This resulted in the title compound (550 mg, yield: 63.5%) as a white solid. LC-MS: (ES, m/z): RT=0.887 min, LCMS: m/z=457 [M+1].

### Step 3: 7-(difluoromethoxy)-4-[3-(2-fluorophenyl)-1-methyl-1H-pyrazol-4-yl]-N-[(4-methoxyphenyl)methyl]pyrido[3,2-d]pyrimidin-6-amine

sodium 2-chloro-2,2-difluoroacetate (166 mg, 1.09 mmol) was added to 4-[3-(2-fluorophenyl)-1-methyl-1H-pyrazol-4-yl]-6-{[(4-methoxyphenyl)methyl]amino}pyrido[3,2-d]pyrimidin-7-ol (250 mg, 547 μmol) and K₂CO₃ (113 mg, 820 μmol) in DMF (10 mL) and H₂O (2 mL) at r.t. The resulting mixture was stirred at 80° C. for 3 h. The mixture was diluted with EA 50 mL and washed with brine 20 mL*2, the organic layer was dried with Na₂SO₄ and concentrated under vacuum. The residue was purified by a silica gel column with DCM:MeOH=18:1. This resulted in the title compound (75 mg, yield: 80%) as a white solid. LC-MS: (ES, m/z): RT=1.234 min, LCMS: m/z=507 [M+1].

### Step 4: 7-(difluoromethoxy)-4-[3-(2-fluorophenyl)-1-methyl-1H-pyrazol-4-yl]pyrido[3,2-d]pyrimidin-6-amine

7-(difluoromethoxy)-4-[3-(2-fluorophenyl)-1-methyl-1H-pyrazol-4-yl]-N-[(4-methoxyphenyl)methyl]pyrido[3,2-d]pyrimidin-6-amine (75 mg, 148 μmol) was added to TFA (5 mL) at r.t. The resulting mixture was stirred at 60° C. for 3 h. The mixture was diluted with EA (150 mL) and washed with brine (70 mL*2), the organic layer was dried with Na₂SO₄ and concentrated under vacuum. The residue was purified by a silica gel column with DCM:MeOH=18:1. This resulted in the title compound (55 mg, 55.5%) as a white solid. LC-MS: (ES, m/z): RT=0.865 min, LCMS: m/z=387 [M+1].

### Step 5: methyl 3-aminobicyclo[1.1.1]pentane-1-carboxylate

TFA (5 mL) was added to methyl 3-{[(tert-butoxy) carbonyl]amino}bicyclo[1.1.1]pentane-1-carboxylate (2 g, 8.1 mmol) in DCM (20 mL) at r.t. The resulting mixture was stirred at r.t for 1 h. The mixture was diluted with DCM 100 mL and washed with Sodium bicarbonate aqueous solution 50 mL*2, the organic layer was dried with Na₂SO₄ and concentrated under vacuum. The residue was purified by a silica gel column with DCM:EA=25:1. This resulted in the title compound (1.2 g, yield: 36%) as a white solid. LC-MS: (ES, m/z): RT=0.653 min, LCMS: m/z=142 [M+1]

Step 6: methyl 3-(dimethylamino) bicyclo[1.1.1]pentane-1-carboxylate: HCHO (254 mg, 10.2 mmol) was added to methyl 3-aminobicyclo[1.1.1]pentane-1-carboxylate (1.2 g, 8.50 mmol) in MeOH (20 mL) and AcOH (2 mL) at r.t for 0.5 h. Then NaBH₃CN (642 mg, 10.2 mmol) was added to the mixture at ice bar. The resulting mixture was stirred at r.t for 2 h. The mixture was diluted with EA 100 mL and washed with brine 50 mL*2, the organic layer was dried with Na₂SO₄ and concentrated under vacuum. The residue was purified by a silica gel column with PE:EA=15:1. This result in the tittle compound (800 mg, yield: 78%) as an off-white solid. LC-MS: (ES, m/z): RT=0.569 min, LCMS: m/z=170 [M+1].

### Step 7:3-(dimethylamino) bicyclo[1.1.1]pentane-1-carboxylic Acid

LiOH (236 mg, 9.44 mmol) was added to methyl 3-(dimethylamino) bicyclo[1.1.1]pentane-1-carboxylate (800 mg, 4.72 mmol) to in THF (16 mL) and H₂O (4 mL) at r.t. The resulting mixture was stirred at 40° C. for 10 h. The mixture was added dilute hydrochloric acid until the PH test paper is alkaline. Then diluted with EA 100 mL and washed with brine 50 mL*2, the organic layer was dried with Na₂SO₄ and concentrated under vacuum. The residue was purified by a silica gel column with DCM:MeOH=18:1. This resulted in the title compound (300 mg, 42.5%) as a yellow solid. LC-MS: (ES, m/z): RT=0.767 min, LCMS: m/z=156 [M+1].

### Step 8: N-[7-(difluoromethoxy)-4-[3-(2-fluorophenyl)-1-methyl-1H-pyrazol-4-yl]pyrido[3,2-d]pyrimidin-6-yl]-3-(dimethylamino) bicyclo[1.1.1]pentane-1-carboxamide

The mixture of Py (2 mL) and T₃P (2 mL), (3R)-1-methylpyrrolidine-3-carboxylic acid (81.6 mg, 632 μmol) and 7-methoxy-4-(1-methyl-3-phenyl-1H-pyrazol-4-yl)quinazolin-6-amine (70 mg, 211 μmol) in THF (5 mL) was stirred at 50° C. for 3 hours.

The mixture was concentrated under vacuum. The residue was purified by a silica gel column with DCM:EA=18:1. The residue was purified by prep-HPLC as following condition: Column: XBridge Prep OBD C18 Column, 30*150 mm, 5 μm; Mobile Phase A: Water (10 mmol/L NH₄HCO₃+0.05% NH; H₂O), Mobile Phase B: ACN: Flow rate: 60 mL/min; Gradient: in 7 min: Wave Length: 220 nm nm: This resulted in the title compound (9.4 mg) as an off-white solid. LC-MS: (ES, m/z): RT=0.662 min, LCMS: m/z=524 [M+1]: 1H NMR (400 MHZ, DMSO-d₆) δ 9.98 (s, 1H), 9.36 (s, 1H), 8.88 (s, 1H), 8.04 (s, 1H), 7.59-7.50 (m, 3H), 7.25 (td, J=7.5, 1.2 Hz, 1H), 7.14 (ddd, J=10.5, 8.3, 1.2 Hz, 1H), 4.03 (s, 3H), 2.16 (s, 6H), 2.09 (s, 6H).

### Example 469: N-{7-cyclopropoxy-4-[3-(2-fluorophenyl)-1-methyl-1H-pyrazol-4-yl]pyrido[3,2-d]pyrimidin-6-yl}-3-(dimethylamino) bicyclo[1.1.1]pentane-1-carboxamide

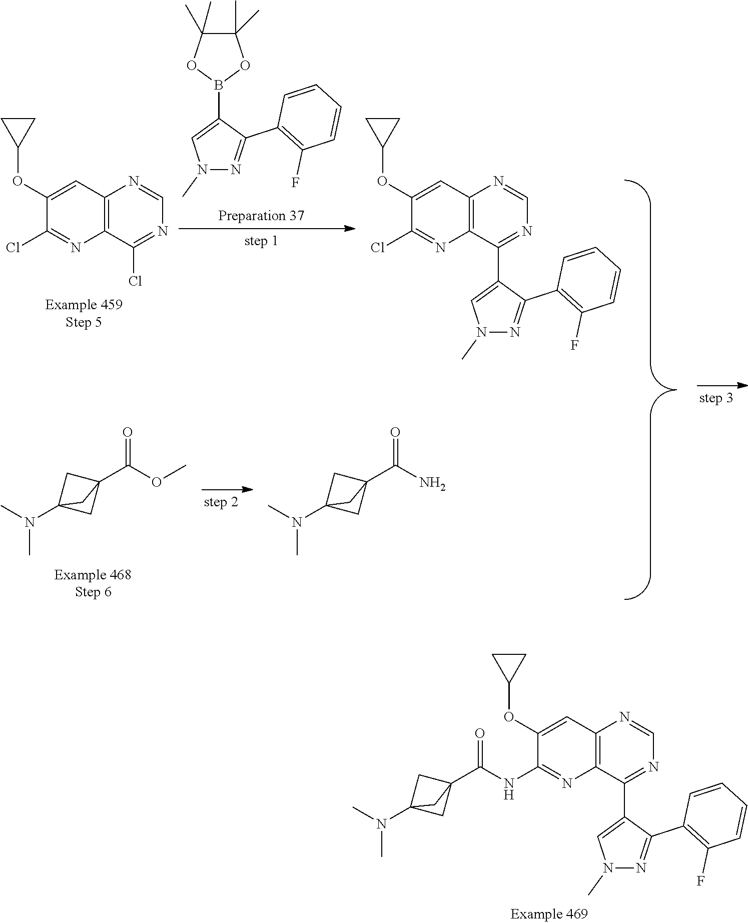

### Step 1: 6-chloro-7-cyclopropoxy-4-(3-(2-fluorophenyl)-1-methyl-1H-pyrazol-4-yl)pyrido[3,2-d]pyrimidine

The reaction mixture of Pd(dppf)cl₂ (35.7 mg, 43.8 μmol), 5-bromo-N-[5-(2-cyclopropyl-2H-1,2,3,4-tetrazol-5-yl)-4-fluoro-2-methylphenyl]pyrazolo[1,5-a]pyridine-3-carboxamide (Product of Example 459, step 5, 200 mg, 438 μmol), 3-(2-fluorophenyl)-1-methyl-4-(4,4,5,5-tetramethyl-1,3,2-dioxaborolan-2-yl)-1H-pyrazole (Preparation 37, 184 mg, 657 μmol) and K₃PO₄ (90.6 mg, 657 μmol) in H₂O (4 mL) and dioxane (16 mL) was stirred at 80° C. for 2 h under N₂. The mixture was diluted with DCM 100 mL and washed with water 50 mL*2, the organic layer was dried with Na₂SO₄ and concentrated under vacuum. The residue was purified by a silica gel column with DCM:MeOH=20:1. This resulted in the title compound (120 mg, yield: 90%) as a white solid. LC-MS: (ES, m/z): RT=0.898 min, LCMS: m/z=396 [M+1].

### Step 2:3-(dimethylamino) bicyclo[1.1.1]pentane-1-carboxamide

NH₃·H₂O (15 mL) was added to methyl 3-(dimethylamino) bicyclo[1.1.1]pentane-1-carboxylate (Example 468, Step 6, 4500 mg, 3.8 mmol) at r.t. The resulting mixture was stirred at r.t for 2 h. The mixture was concentrated under vacuum. This resulted in the title compound (500 mg, yield: 91%) as a white solid. LC-MS: (ES, m/z): RT=0.736 min, LCMS: m/z=155 [M+1].

### Step 3: N-{7-cyclopropoxy-4-[3-(2-fluorophenyl)-1-methyl-1H-pyrazol-4-yl]pyrido[3,2-d]pyrimidin-6-yl}-3-(dimethylamino) bicyclo[1.1.1]pentane-1-carboxamide

A mixture of 4-{6-chloro-7-cyclopropoxypyrido[3,2-d]pyrimidin-4-yl}-3-(2-fluorophenyl)-1-methyl-1H-pyrazole (60 mg, 151 μmol), 3-(dimethylamino) bicyclo[1.1.1]pentane-1-carboxamide (46.5 mg, 302 μmol), XantPhos (17.4 mg, 30.2 μmol), Pd₂(dba)₃ (13.8 mg, 15.1 μmol) and K₂CO₃ (302 μmol, 41.9 mg) in dioxane (6 mL) was heated at 100° C. for 3 h under N₂. The mixture was concentrated under vacuum. The residue was purified by Prep-TLC with DCM:MeOH=25:1 and further purified by Prep-HPLC as following conditions: Column: YMC-Actus Triart C18, 30*150 mm, 5 um; Mobile Phase A: Water (10 mmol/L NH₄HCO₃+0.1% NH₃·H₂O), Mobile Phase B: ACN: Flow rate: 60 mL/min; Gradient: 40% B to 55% B in 8 min, 55% B; Wave Length: 254 nm. This resulted in the title compound (9.7 mg) as an off-white solid. LC-MS: (ES, m/z): RT=1.049 min, LCMS: m/z=514 [M+1]; ¹H NMR (400 MHZ, DMSO-d₆) δ 9.56 (s, 1H), 9.39 (s, 1H), 8.76 (s, 1H), 7.91 (s, 1H), 7.52 (td, J=7.5, 1.9 Hz, 1H), 7.48-7.38 (m, 1H), 7.25 (td, J=7.5, 1.2 Hz, 1H), 7.20-7.10 (m, 1H), 4.28 (dt, J=6.0, 3.0 Hz, 1H), 4.01 (s, 3H), 2.16 (s, 6H), 2.08 (s, 6H), 0.95 (t, J=5.6 Hz, 4H).

### Example 470:6-{[(4-methoxyphenyl)methyl]amino}-4-(1-methyl-3-phenyl-1H-pyrazol-4-yl)pyrido[3,2-d]pyrimidin-7-ol

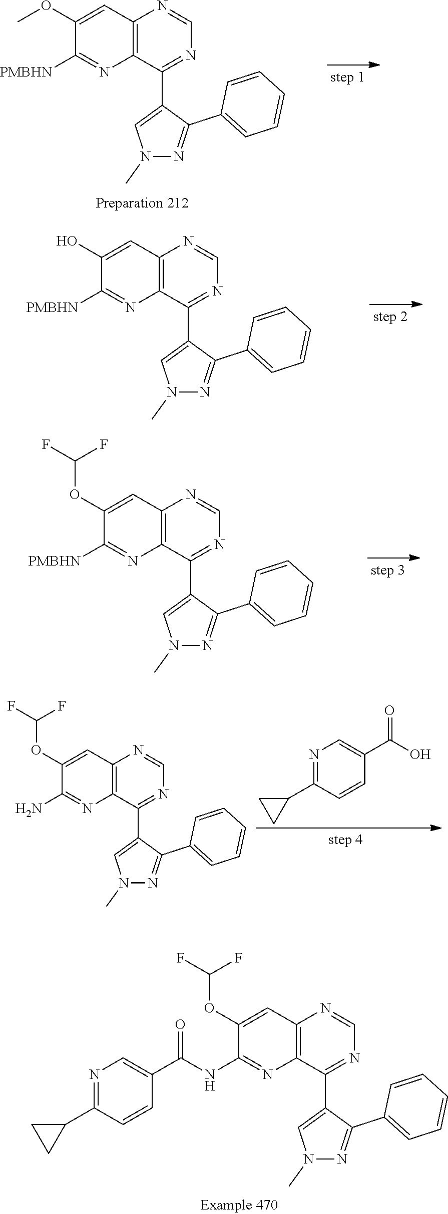

### Step 1: 6-{[(4-methoxyphenyl)methyl]amino}-4-(1-methyl-3-phenyl-1H-pyrazol-4-yl)pyrido[3,2-d]pyrimidin-7-ol

L-Selectride (678 mg, 2.64 mmol) was added to, 7-methoxy-N-[(4-methoxyphenyl)methyl]-4-(1-methyl-3-phenyl-1H-pyrazol-4-yl)pyrido[3,2-d]pyrimidin-6-amine (Preparation 212, 800 m g, 1.76 mmol) in THF (20 mL) at r.t. The resulting mixture was stirred at 80° C. for 2 h. The mixture was concentrated under vacuum. The residue was purified by a silica gel column with DCM:EA=25:1. This resulted in the title compound (550 mg, yield: 67%) as a white solid. LC-MS: (ES, m/z): RT=0.997 min, LCMS: m/z=439 [M+1].

### Step 2:7-(difluoromethoxy)-N-[(4-methoxyphenyl)methyl]-4-(1-methyl-3-phenyl-1H-pyrazol-4-yl)pyrido[3,2-d]pyrimidin-6-amine

sodium 2-chloro-2,2-difluoroacetate (362 mg, 2.38 mmol) was added to the mixture of 6-{[(4-methoxyphenyl)methyl]amino}-4-(1-methyl-3-phenyl-1H-pyrazol-4-yl)pyrido[3,2-d]pyrimidin-7-ol (700 mg, 1.59 mmol) and K₂CO₃ (328 mg, 2.38 mmol) in DMF (20 mL) and H₂O (5 mL) at r.t. The resulting mixture was stirred at 80° C. for 3 h under N₂. The mixture was diluted with EA 50 mL and washed with brine 30 mL*2, the organic layer was dried with Na₂SO₄ and concentrated under vacuum. The residue was purified by a silica gel column with DCM:MeOH=18:1. This resulted in the title compound (300 mg, yield: 60%) as a white solid. LC-MS: (ES, m/z): RT=1.120 min, LCMS: m/z=489 [M+1].

### Step 3: 7-(difluoromethoxy)-4-(1-methyl-3-phenyl-1H-pyrazol-4-yl)pyrido[3,2-d]pyrimidin-6-amine

The reaction mixture of 7-(difluoromethoxy)-N-[(4-methoxyphenyl)methyl]-4-(1-methyl-3-phenyl-1H-pyrazol-4-yl)pyrido[3,2-d]pyrimidin-6-amine (300 mg, 614 μmol) in TFA (15 mL) was stirred at 60° C. for 3 h. The mixture was concentrated under vacuum. The mixture was diluted with DCM 100 mL and washed with sodium bicarbonate aqueous solution 50 mL*2, the organic layer was dried with Na₂SO₄ and concentrated under vacuum. The residue was purified by a silica gel column with DCM:MeOH=18:1. This resulted in the title compound (200 mg, 50%) as a white solid. LC-MS: (ES, m/z): RT=0.903 min, LCMS: m/z=369 [M+1].

### Step 4:6-cyclopropyl-N-[7-(difluoromethoxy)-4-(1-methyl-3-phenyl-1H-pyrazol-4-yl)pyrido[3,2-d]pyrimidin-6-yl]pyridine-3-carboxamide

2,4,6-trichlorobenzoyl chloride (58 mg, 151 μmol) was added to TEA (46.5 mg, 405 μmol) and 6-cyclopropylpyridine-3-carboxylic acid (44.0 mg, 270 μmol) in THF (10 mL) at ice bath for 1 h. Then 7-(difluoromethoxy)-4-(1-methyl-3-phenyl-1H-pyrazol-4-yl)pyrido[3,2-d]pyrimidin-6-amine (50 mg, 135 μmol) added to the mixture at r.t. The resulting mixture was stirred at 80° C. for 4 h. The mixture was diluted with EA 60 mL and washed with brine 30 mL*2, the organic layer was dried with Na₂SO₄ and concentrated under vacuum. The residue was purified by a silica gel column with PE:EA=15:1. The residue was purified by prep-HPLC as following condition: Column: XBridge Shield RP18 OBD Column, 30*150 mm, 5 μm; Mobile Phase A: Water (10 mmol/L NH₄HCO₃), Mobile Phase B: ACN: Flow rate: 60 mL/min; Gradient: 35% B to 60% B in 8 min: Wave Length: 220/254 nm: This resulted in the title compound (9.7 mg) as an off-white solid. LC-MS: (ES, m/z): RT=0.826 min, LCMS: m/z=514 [M+1]; ¹H NMR (400 MHZ, DMSO-d₆) δ 11.08 (s, 1H), 9.01 (s, 3H), 8.21 (dd, J=8.2, 2.3 Hz, 1H), 8.12 (s, 1H), 7.58 (s, 4H), 7.55-7.46 (m, 3H), 7.34-7.28 (m, 3H), 3.99 (s, 3H), 2.24 (ddd, J=12.6, 8.1, 5.1 Hz, 1H), 1.05 (dtd, J=10.1, 8.2, 5.3 Hz, 4H).

### Example 471—Intentionally Omitted

### Example 472: 6-(7-ethyl-2H-indazol-4-yl)-7-methoxy-4-(1-methyl-3-phenyl-1H-pyrazol-4-yl)quinazoline

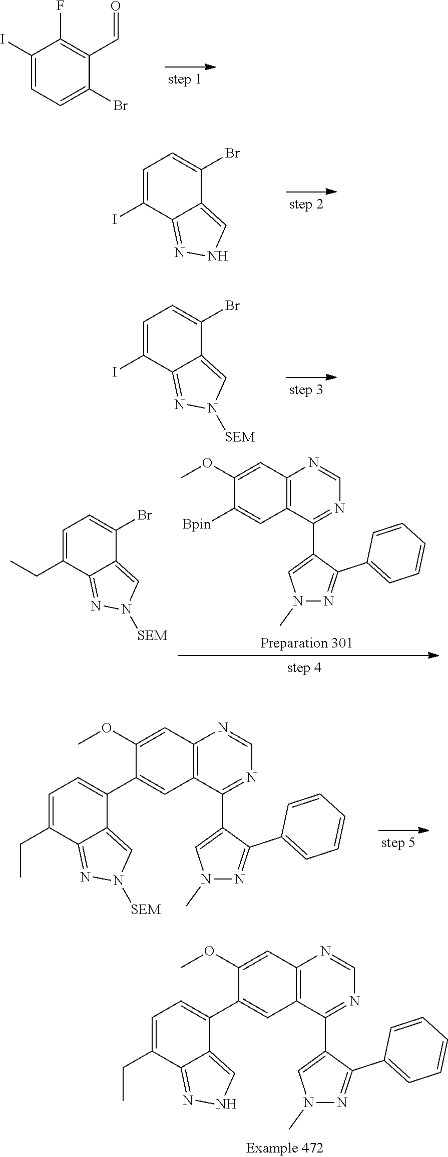

### Step 1: 4-bromo-7-iodo-2H-indazole

N₂H₄·H₂O (2.54 g, 21.2 mmol) was added to 6-bromo-2-fluoro-3-iodobenzaldehyde (1 g, 3.04 mmol) in the DMSO (16 mL) at rt. The resulting mixture was stirred at 120° C. for 2 h. The mixture was diluted with EA 100 mL and washed with brine 50 mL*2, the organic layer was dried with Na₂SO₄ and concentrated under vacuum. The residue was purified by a silica gel column with PE:EA=15:1. This result in the title compound (850 mg, 83%) as an off-white solid. LC-MS: (ES, m/z): RT=1.121 min, LCMS: m/z=323 [M+1].

### Step 2: 4-bromo-7-iodo-2-{[2-(trimethylsilyl)ethoxy]methyl}-2H-indazole

NaH (148 mg, 3.70 mmol) was added to 4-bromo-7-iodo-2H-indazole (600 mg, 1.85 mmol) in DMF (10 mL) at 0° C. . . . The resulting mixture was stirred at 0° C. for 0.5 h. Then SEM-Cl (462 mg, 2.77 mmol) was added to the mixture at 0° C. The resulting mixture was stirred at rt for 2 h. The mixture was diluted with DCM 100 mL and water 50 mL*2, the organic layer was dried with Na₂SO₄ and concentrated under vacuum. The residue was purified by a silica gel column with DCM:EA=25:1. This resulted in the title compound (300 mg, yield: 49%) as a white solid. LC-MS: (ES, m/z): RT=0.988 min, LCMS: m/z=453 [M+1].

### Step 3: 4-bromo-7-ethyl-2-{[2-(trimethylsilyl)ethoxy]methyl}-2H-indazole

The reaction mixture of 4-bromo-7-iodo-2-{[2-(trimethylsilyl)ethoxy]methyl}-2H-indazole (300 mg, 661 μmol), Et₂ZN (206 mg, 661 μmol) and Pd(dppf)cl₂ (53.9 mg, 66.1 μmol) in toluene (10 mL) was stirred at 60° C. for 3 h under N₂. The mixture was diluted with EA 100 mL and washed with brine 50 mL*2, the organic layer was dried with Na₂SO₄ and concentrated under vacuum. The residue was purified by a silica gel column with DCM:EA=15:1. This resulted in the title compound (200 mg, yield: 53%) as a light yellow solid. LC-MS: (ES, m/z): RT=0.795 min, LCMS: m/z=355 [M+1].

### Step 4: 6-(7-ethyl-2-((2-(trimethylsilyl)ethoxy)methyl)-2H-indazol-4-yl)-7-methoxy-4-(1-methyl-3-phenyl-1H-pyrazol-4-yl)quinazoline

The reaction mixture of 4-bromo-7-ethyl-2-{[2-(trimethylsilyl)ethoxy]methyl}-2H-indazole (200 mg, 562 μmol), 7-methoxy-4-(1-methyl-3-phenyl-1H-pyrazol-4-yl)-6-(4,4,5,5-tetramethyl-1,3,2-dioxaborolan-2-yl)quinazoline (Preparation 301, 248 mg, 562 μmol), Pd(dppf)Cl₂ (41.1 mg, 56.2 μmol) and K₂CO₃ (116 mg, 843 μmol) in dioxane/H₂O (8 mL/2 mL) was stirred at 80° C. for 2 h under N₂. The mixture was diluted with EA 50 mL and washed with brine 30 mL*2, the organic layer was dried with Na₂SO₄ and concentrated under vacuum. The residue was purified by a silica gel column with DCM:MeOH=20:1. This resulted in the title compound (100 mg, yield: 83%) as a yellow solid. LC-MS: (ES, m/z): RT=1.132 min, LCMS: m/z=591 [M+1].

### Step 5: 6-(7-ethyl-2H-indazol-4-yl)-7-methoxy-4-(1-methyl-3-phenyl-1H-pyrazol-4-yl)quinazoline

TFA (2 mL) was added to 6-(7-ethyl-2-{[2-(trimethylsilyl)ethoxy]methyl}-2H-indazol-4-yl)-7-methoxy-4-(1-methyl-3-phenyl-1H-pyrazol-4-yl)quinazoline (100 mg, 169 μmol) in DCM (6 mL) at r.t. The resulting mixture was stirred at r.t for 2 h. The mixture was concentrated under vacuum and diluted with DCM 100 mL and washed with sodium bicarbonate aqueous solution 50 mL*2, the organic layer was dried with Na₂SO₄ and concentrated under vacuum. The residue was purified by a silica gel column with DCM:EA=25:1. The residue was purified by prep-HPLC as following condition: Column: XBridge Shield RP18 OBD Column, 30*150 mm, 5 um; Mobile Phase A: Water (10 mmol/L NH₄HCO₃+0.1% NH₃·H₂O), Mobile Phase B: ACN; Flow rate: 60 mL/min; Gradient: 28% B to 61% B in 7 min: Wave Length: 254/220 nm: This resulted in the title compound (26.3 mg) as a white solid. LC-MS: (ES, m/z): RT=0.840 min, LCMS: m/z=461 [M+1]: 1H NMR (400 MHZ, DMSO-d₆) δ 13.17 (s, 1H), 9.18 (s, 1H), 8.25 (s, 1H), 7.68 (s, 1H), 7.61-7.51 (m, 2H), 7.34-7.24 (m, 5H), 7.10 (d, J=7.2 Hz, 1H), 6.47 (d, J=7.1 Hz, 1H), 3.94 (d, J=10.1 Hz, 6H), 2.92 (q, J=7.5 Hz, 2H), 1.30 (t, J=7.5 Hz, 3H).

### Example 473—Intentionally Omitted

### Example 474: N-{4-[3-(4-chlorophenyl)-1-methyl-1H-pyrazol-4-yl]-7-methoxypyrido[3,2-d]pyrimidin-6-yl}-3-(dimethylamino) bicyclo[1.1.1]pentane-1-carboxamide

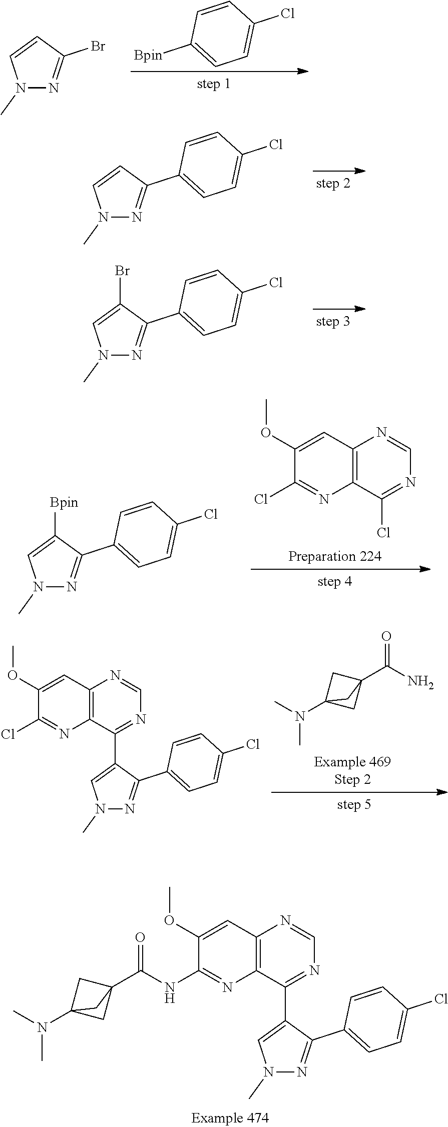

### Step 1: 3-(4-chlorophenyl)-1-methyl-1H-pyrazole

The reaction mixture of Pd(pddf)Cl₂ (1.01 g, 1.24 mmol), K₂CO₃ (2.56 g, 18.6 mmol), 3-bromo-1-methyl-1H-pyrazole (2 g, 12.4 mmol) and 2-(4-chlorophenyl)-4,4,5,5-tetramethyl-1,3,2-dioxaborolane (3.53 g, 14.8 mmol) in dioxane/H₂O (40 mL/10 mL) was heated at 80° C. for 16 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with PE:EA=3:1. The title compound (1.74 g) was collected as a white solid. LC-MS: (ES, m/z): RT=0.670 min, LCMS: m/z=193 [M+1],

### Step 2: 4-bromo-3-(4-chlorophenyl)-1-methyl-1H-pyrazole

NBS (1.86 g, 10.5 mmol) was added to 3-(4-chlorophenyl)-1-methyl-1H-pyrazole (1.573 g, 8.14 mmol) in DMF (10 mL) at rt. The resulting mixture was stirred at rt for 3 h. The reaction mixture was diluted with EtOAc (120 mL), and washed sequentially with water (60 mL). The organic layer was dried over Na₂SO₄, filtered and concentrated under vacuum. The crude product was purified by Prep-TLC with PE:EA=5:1. The title compound (2.10 g) was collected as a white solid. LC-MS: (ES, m/z): RT=0.918 min, LCMS: m/z=273 [M+1].

### Step 3: 3-(4-chlorophenyl)-1-methyl-4-(4,4,5,5-tetramethyl-1,3,2-dioxaborolan-2-yl)-1H-pyrazole

The reaction mixture of Pd(pddf)Cl₂ (600 mg, 736 μmol), AcOK (1.08 g, 11.0 mmol), 4-bromo-3-(4-chlorophenyl)-1-methyl-1H-pyrazole (2 g. 7.36 mmol) and 4,4,5,5-tetramethyl-2-(4,4,5,5-tetramethyl-1,3,2-dioxaborolan-2-yl)-1,3,2-dioxaborolane (3.73 g, 14.7 mmol) in DMSO (10 mL) was heated at 80° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:MeOH=30:1. The title compound (943 mg) was collected as a white solid. LC-MS: (ES, m/z): RT=0.800 min, LCMS: m/z=319 [M+1].

### Step 4: 4-{6-chloro-7-methoxypyrido[3,2-d]pyrimidin-4-yl}-3-(4-chlorophenyl)-1-methyl-1H-pyrazole

The reaction mixture of Pd(pddf)Cl₂ (264 mg, 324 μmol), K₃PO₄ (724 mg, 3.40 mmol), 4,6-dichloro-7-methoxypyrido[3,2-d]pyrimidine (Preparation 224, 746 mg, 3.24 mmol) and 3-(4-chlorophenyl)-1-methyl-4-(4,4,5,5-tetramethyl-1,3,2-dioxaborolan-2-yl)-1H-pyrazole (927 mg, 2.91 mmol) in dioxane/H₂O (9 mL/3 mL) was heated at 60° C. for 16 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:EA=1:1. The title compound (432 mg) was collected as a white solid. LC-MS: (ES, m/z): RT=0.692 min, LCMS: m/z=386 [M+1].

### Step 5: N-{4-[3-(4-chlorophenyl)-1-methyl-1H-pyrazol-4-yl]-7-methoxypyrido[3,2-d]pyrimidin-6-yl}-3-(dimethylamino) bicyclo[1.1.1]pentane-1-carboxamide

The reaction mixture of 4-{6-chloro-7-methoxypyrido[3,2-d]pyrimidin-4-yl}-3-(4-chlorophenyl)-1-methyl-1H-pyrazole (200 mg, 517 μmol), 3-(dimethylamino) bicyclo[1.1.1]pentane-1-carboxamide (Product of Example 469 Step 2, 158 mg, 1.03 mmol), XantPhos (59.3 mg, 103 μmol), Pd₂(dba)₃ (53.5 mg, 51.7 μmol) and Cs₂CO₃ (252 mg, 775 μmol) in dioxane (10 mL) was heated at 100° C. for 3 h under N₂. The reaction mixture was diluted with EtOAc (120 mL), and washed sequentially with water (60 mL). The organic layer was dried over Na₂SO₄, filtered and concentrated under vacuum. The crude product was purified by Prep-TLC with DCM:MeOH=20:1, filtered and evaporated to afford crude product. The title compound (32.1 mg) was collected as an off-white solid. LC-MS: (ES, m/z): RT=0.640 min, LCMS: m/z=504 [M+1]; ¹H NMR (400 MHZ, DMSO-d₆) δ 9.55 (s, 1H), 9.47 (s, 1H), 8.85 (s, 1H), 7.72 (s, 1H), 7.60-7.52 (m, 2H), 7.43-7.37 (m, 2H), 4.09 (s, 3H), 3.99 (s, 3H), 2.15 (s, 6H), 2.09 (s, 6H).

### Example 475:7-(difluoromethoxy)-6-{[(3R,4R)-3-fluoro-1-methylpiperidin-4-yl]oxy}-4-(1-methyl-3-phenyl-1H-pyrazol-4-yl)quinazoline

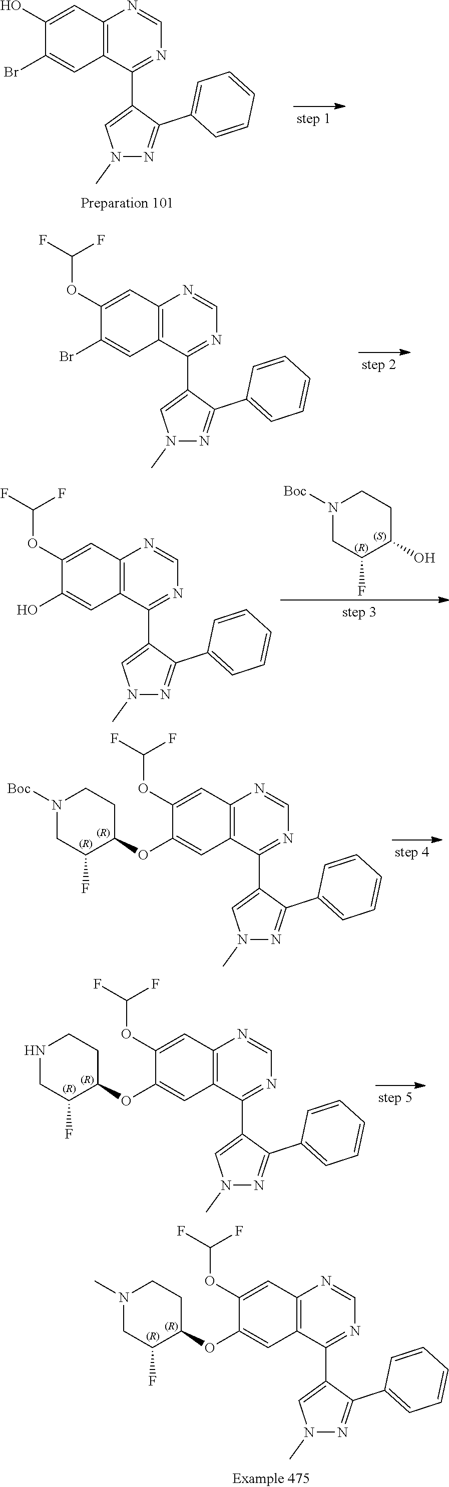

### Step 1: 6-bromo-7-(difluoromethoxy)-4-(1-methyl-3-phenyl-1H-pyrazol-4-yl)quinazoline

The reaction mixture of KF (121 mg, 2.09 mmol), KI (260 mg, 1.57 mmol), 6-bromo-4-(1-methyl-3-phenyl-1H-pyrazol-4-yl)quinazolin-7-ol (Preparation 101, 200 mg, 524 μmol) and diethyl (bromodifluoromethyl)phosphonate (419 mg, 1.57 mmol) in MeCN (15 mL) at rt. The resulting mixture was stirred at rt for 16 h. The reaction mixture was diluted with EA (50 mL), and washed with water (30 mL*3) and saturated brine (100 mL*1). The organic layer was dried over Na₂SO₄, filtered and evaporated to afford crude product. The crude product was purified by Prep-TLC with DCM:MeOH=10:1, to afford the title compound (40 mg, yield: 50%) as a yellow solid. LC-MS: (ES, m/z): RT=1.229 min, LCMS: m/z=431 [M+1],

### Step 2: 7-(difluoromethoxy)-4-(1-methyl-3-phenyl-1H-pyrazol-4-yl)quinazolin-6-ol

The reaction mixture of BrettPhos Pd G3 (33.5 mg, 37.0 μmol), 6-bromo-7-(difluoromethoxy)-4-(1-methyl-3-phenyl-1H-pyrazol-4-yl)quinazoline (80 mg, 185 μmol) and Cs₂CO₃ (241 mg, 740 μmol) in dioxane/H₂O (4 mL/1 mL) was heated at 100° C. for 4 h under N₂. The reaction mixture was diluted with EA (50 mL) and washed with water (20 mL*3) and saturated brine (20 mL*1). The organic layer was dried over Na₂SO₄, filtered and evaporated to afford crude product. The crude product was purified by Prep-TLC with DCM:MeOH=20:1, to afford the title compound (40 mg, yield: 58.7%) as a brown solid. LC-MS: (ES, m/z): RT=0.524 min, LCMS: m/z=369 [M+1].

### Step 3: tert-butyl (3R,4R)-4-{[7-(difluoromethoxy)-4-(1-methyl-3-phenyl-1H-pyrazol-4-yl)quinazolin-6-yl]oxy}-3-fluoropiperidine-1-carboxylate

DIAD (43.6 mg, 216 μmol) was added to PPh₃ (84.8 mg, 324 μmol), 7-(difluoromethoxy)-4-(1-methyl-3-phenyl-1H-pyrazol-4-yl)quinazolin-6-ol (40 mg, 108 μmol) and tert-butyl (3R,4S)-3-fluoro-4-hydroxypiperidine-1-carboxylate (71.0 mg, 324 μmol) in THF (5 mL) at 0° C. The resulting mixture was stirred at rt for 16 h. The reaction mixture was diluted with EA (50 mL), and washed with water (50 mL*3) and saturated brine (50 mL*1). The organic layer was dried over Na₂SO₄, filtered and evaporated to afford crude product. The crude product was purified by Prep-TLC with DCM:MeOH=10:1 to afford the title compound (30 mg, yield: 48.7%) as a brown solid. LC-MS: (ES, m/z): RT=1.392 min, LCMS: m/z=570 [M+1].

### Step 4: 7-(difluoromethoxy)-6-{[(3R,4R)-3-fluoropiperidin-4-yl]oxy}-4-(1-methyl-3-phenyl-1H-pyrazol-4-yl)quinazoline

TFA (0.1 mL) was added to tert-butyl (3R,4R)-4-{[7-(difluoromethoxy)-4-(1-methyl-3-phenyl-1H-pyrazol-4-yl)quinazolin-6-yl]oxy}-3-fluoropiperidine-1-carboxylate (30 mg, 52.6 μmol) in DCM (0.4 mL) at rt. The resulting mixture was hold to room temperature for 2 h. The reaction mixture was diluted with EA (50 mL), and washed with saturated aqueous NaHCO₃ (50 mL*3) and saturated brine (50 mL*1). The organic layer was dried over Na₂SO₄, filtered and evaporated to afford 20 mg crude product. This crude product was used to the next step without purification. LC-MS: (ES, m/z): RT=0.805 min, LCMS: m/z=470 [M+1].

### Step 5: 7-(difluoromethoxy)-6-{[(3R,4R)-3-fluoro-1-methylpiperidin-4-yl]oxy}-4-(1-methyl-3-phenyl-1H-pyrazol-4-yl)quinazoline

NaCNBH₃ (5.35 mg, 85.2 μmol) was added to 7-(difluoromethoxy)-6-{[(3R,4R)-3-fluoropiperidin-4-yl]oxy}-4-(1-methyl-3-phenyl-1H-pyrazol-4-yl)quinazoline (20 mg, 42.6 μmol) and HCHO (12.7 mg, 213 μmol) in MeOH/CH₃COOH (1 mL/0.1 mL) at 0° C. The resulting mixture was stirred at rt for 1 h. The reaction mixture was diluted with EA (50 mL), and washed with water (50 mL*3) and saturated brine (30 mL*1). The organic layer was dried over Na₂SO₄, filtered and evaporated to afford crude product. The crude product was purified by Prep-TLC with DCM:MeOH=20:1. The residue was purified by Prep-HPLC as following condition: Column: XBridge Shield RP18 OBD Column, 30*150 mm, 5 um; Mobile Phase A: Water (10 mmol/L NH₄HCO₃+0.1% NH₃·H₂O), Mobile Phase B: ACN: Flow rate: 60 mL/min; Gradient: 24% B to 57% B in 7 min, 57% B; Wave Length: 254/220 nm, to afford the title compound (2.2 mg, yield: 10.7%) as a white solid. LC-MS: (ES, m/z): RT=1.175 min, LCMS: m/z=484 [M+1]; ¹H NMR (400 MHZ, DMSO-d₆) δ 9.21 (s, 1H), 8.32 (s, 1H), 7.73 (d, J=10.6 Hz, 1H), 7.70-7.50 (m, 1H), 7.38-7.30 (m, 2H), 7.29-7.24 (m, 4H), 4.70-4.66 (m, 1H), 4.07 (s, 3H), 3.95 (s, 1H), 3.01 (s, 1H), 2.60 (s, 1H), 2.31-2.08 (m, 5H), 1.62 (s, 1H), 1.41 (d, J=11.1 Hz, 1H).

### Example 476: 7-cyclopropoxy-6-(((3R,4R)-3-fluoro-1-methylpiperidin-4-yl)oxy)-4-(1-methyl-3-phenyl-1H-pyrazol-4-yl)quinazoline

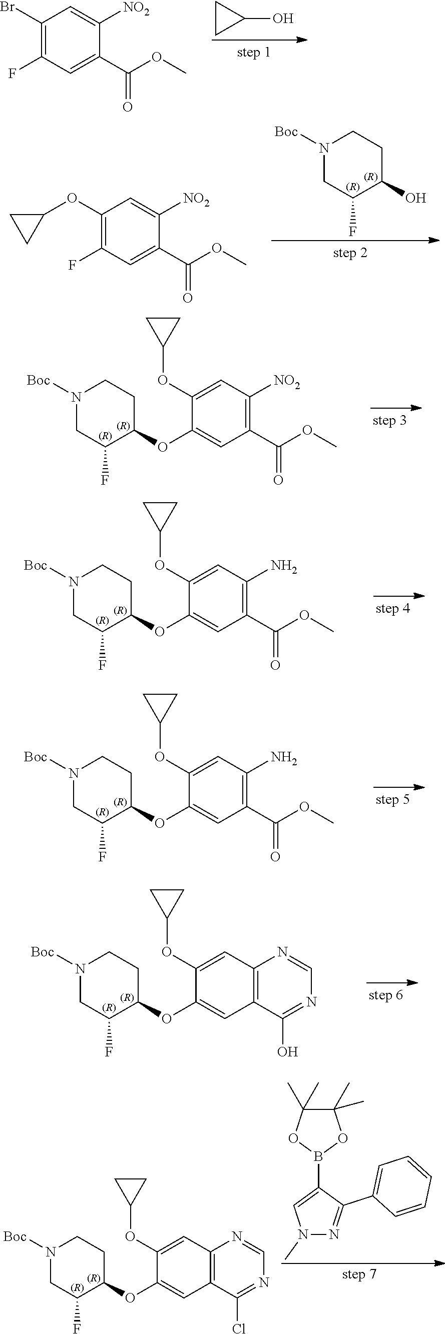

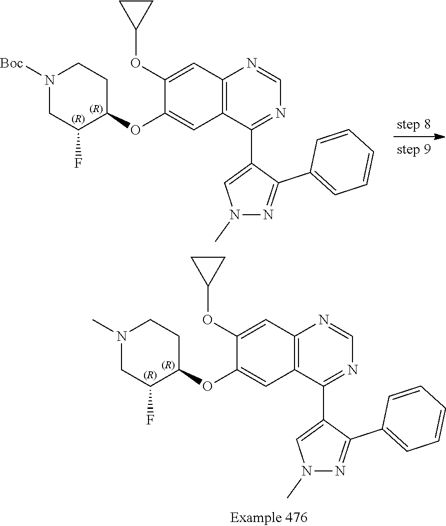

### Step 1: methyl 4-cyclopropoxy-5-fluoro-2-nitrobenzoate

The reaction mixture of methyl 4-bromo-5-fluoro-2-nitrobenzoate (4 g, 14.3 mmol), cyclopropanol (4.15 g, 71.5 mmol), RockPhos Pd G₃ (1.19 g, 1.43 mmol) and Cs₂CO₃ (9.32 g, 28.6 mmol) in toluene (40 mL) at rt. The resulting mixture was heated at 100° C. for 16 h. The reaction mixture was diluted with EA (100 mL), and washed with water (100 mL*3) and saturated brine (100 mL*1). The organic layer was dried over Na₂SO₄, filtered and evaporated to afford crude product. The crude product was purified by Prep-TLC with PE:EA=4:1 to afford the title compound (600 mg, yield: 16.4%) as a brown solid. LC-MS: (ES, m/z): RT=1.139 min, LCMS: m/z=256 [M+1],

### Step 2: tert-butyl (3R,4R)-4-[2-cyclopropoxy-5-(methoxycarbonyl)-4-nitrophenoxy]-3-fluoropiperidine-1-carboxylate

NaH (94.0 mg, 2.35 mmol) was added to methyl 4-cyclopropoxy-5-fluoro-2-nitrobenzoate (600 mg, 2.35 mmol) and tert-butyl (3R,4R)-3-fluoro-4-hydroxypiperidine-1-carboxylate (618 mg, 2.82 mmol) in DMF (20 mL) at 0° C. The resulting mixture was heated to 50° C. for 2 h. The reaction mixture was diluted with EA (150 mL), and washed with water (150 mL*3) and saturated brine (150 mL*1). The organic layer was dried over Na₂SO₄, filtered and evaporated to afford 300 mg crude product as a brown solid. This crude product was used to the next step without purification. LC-MS: (ES, m/z): RT=1.271 min, LCMS: m/z=399.2 [M-56].

### Step 3: tert-butyl (3R,4R)-4-[4-amino-2-cyclopropoxy-5-(methoxycarbonyl) phenoxy]-3-fluoropiperidine-1-carboxylate

The reaction mixture of Fe (324 mg, 5.80 mmol), NH₄Cl (313 mg, 5.80 mmol) and tert-butyl (3R,4R)-4-[2-cyclopropoxy-5-(methoxycarbonyl)-4-nitrophenoxy]-3-fluoropiperidine-1-carboxylate (330 mg, 726 μmol) in EtOH/H₂O (8 mL/2 mL) at rt. The resulting mixture was heated to 80° C. for 2 h. The mixture was filtered through a Celite pad and the filtrate was concentrated under vacuum. The crude product was purified by Prep-TLC with DCM:MeOH=20:1 to afford the title compound (260 mg, yield: 84.4%) as a yellow solid. LC-MS: (ES, m/z): RT=1.243 min, LCMS: m/z=369.2 [M-58],

### Step 4: 2-amino-5-{[(3R,4R)-1-[(tert-butoxy) carbonyl]-3-fluoropiperidin-4-yl]oxy}-4-cyclopropoxybenzoic Acid

LiOH (88.0 mg, 3.67 mmol) was added to tert-butyl (3R,4R)-4-[4-amino-2-cyclopropoxy-5-(methoxycarbonyl) phenoxy]-3-fluoropiperidine-1-carboxylate (260 mg, 612 μmol) in MeOH/H₂O (8 mL/2 mL) at rt. The resulting mixture was stirred at rt for 4 h. The mixture was concentrated under vacuum. The crude product was purified by Prep-TLC with PE:EA=4:1 to afford the title compound (210 mg, yield: 83.6%) as a yellow solid. LC-MS: (ES, m/z): RT=1.104 min, LCMS: m/z=355 [M-58].

### Step 5: tert-butyl (3R,4R)-4-[(7-cyclopropoxy-4-hydroxyquinazolin-6-yl)oxy]-3-fluoropiperidine-1-carboxylate

The reaction mixture of 2-amino-5-{[(3R,4R)-1-[(tert-butoxy) carbonyl]-3-fluoropiperidin-4-yl]oxy}-4-cyclopropoxybenzoic acid (210 mg, 511 μmol) and formamidine acetic acid salt (20.1 mg, 194 μmol) in EtOH (10 mL) at rt. The resulting mixture was heated to 80° C. for 16 h. The resulting solution was diluted with water (200 mL*2), the solid was collected by filtration, and the title compound (200 mg, yield: 93.4%) was collected as a white solid. LC-MS: (ES, m/z): RT=1.055 min, LCMS: m/z=420 [M+1].

### Step 6: tert-butyl (3R,4R)-4-[(4-chloro-7-cyclopropoxyquinazolin-6-yl)oxy]-3-fluoropiperidine-1-carboxylate

POCl₃ (383 mg, 2.50 mmol) was added to tert-butyl (3R,4R)-4-[(7-cyclopropoxy-4-hydroxyquinazolin-6-yl)oxy]-3-fluoropiperidine-1-carboxylate (210 mg, 500 μmol) and N,N-diethylaniline (525 mg, 3.50 mmol) in MeCN (10 mL) at rt. The resulting mixture was heated to 80° C. for 2 h. The resulting solution was added to ice-water, then NaHCO₃ (1.00 M) was added until pH=8. The solution was extracted with EA and concentrated under vacuum. The residue was purified by a silica gel column with DCM:EA=3:1 to afford the title compound (200 mg, yield: 91.7%) as a yellow solid. LC-MS: (ES, m/z): RT=1.094 min, LCMS: m/z=438.2 [M+1].

### Step 7: tert-butyl (3R,4R)-4-{[7-cyclopropoxy-4-(1-methyl-3-phenyl-1H-pyrazol-4-yl)quinazolin-6-yl]oxy}-3-fluoropiperidine-1-carboxylate

The reaction mixture of Pd(dppf)Cl₂ (33.3 mg, 45.6 μmol), K₂CO₃ (94.3 mg, 684 μmol), tert-butyl (3R,4R)-4-[(4-chloro-7-cyclopropoxyquinazolin-6-yl)oxy]-3-fluoropiperidine-1-carboxylate (200 mg, 456 μmol) and 1-methyl-3-phenyl-4-(4,4,5,5-tetramethyl-1,3,2-dioxaborolan-2-yl)-1H-pyrazole (155 mg, 547 μmol) dioxane/H₂O (8 mL/2 mL) was heated to 80° C. for 2 h under N₂. The reaction mixture was diluted with EA (100 mL), and washed with water (100 mL*3) and saturated brine (100 mL*1). The organic layer was dried over Na₂SO₄, filtered and evaporated to afford crude product. The crude product was purified by Prep-TLC with DCM:MeOH=20:1 to afford the title compound (40 mg, yield: 50%) as a brown solid. LC-MS: (ES, m/z): RT=1.171 min, LCMS: m/z=560.2 [M+1].

### Step 8:7-cyclopropoxy-6-{[(3R,4R)-3-fluoropiperidin-4-yl]oxy}-4-(1-methyl-3-phenyl-1H-pyrazol-4-yl)quinazoline

TFA (1 mL) was added to tert-butyl (3R,4R)-4-{[7-cyclopropoxy-4-(1-methyl-3-phenyl-1H-pyrazol-4-yl)quinazolin-6-yl]oxy}-3-fluoropiperidine-1-carboxylate (150 mg, 268 μmol) in DCM (4 mL) at rt. The resulting mixture was stirred at rt for 2 h. The reaction mixture was evaporated and then diluted with EA (50 mL), and washed with saturated aqueous NaHCO₃ (50 mL*3) and saturated brine (50 mL*1). The organic layer was dried over Na₂SO₄, filtered and evaporated to afford 110 mg crude product. This crude product was used to the next step without purification. LC-MS: (ES, m/z): RT=0.797 min, LCMS: m/z=460.2 [M+1].

### Step 9:7-cyclopropoxy-6-(((3R,4R)-3-fluoro-1-methylpiperidin-4-yl)oxy)-4-(1-methyl-3-phenyl-1H-pyrazol-4-yl)quinazoline

NaCNBH₃ (27.3 mg, 434 μmol) was added to HCHO (32.4 mg, 1.08 mmol) and 7-cyclopropoxy-6-{[(3R,4R)-3-fluoropiperidin-4-yl]oxy}-4-(1-methyl-3-phenyl-1H-pyrazol-4-yl)quinazoline (100 mg, 217 μmol) in MeOH (5 mL) at 0° C. The resulting mixture was stirred at rt for 2 h. The reaction mixture was diluted with EA (50 mL), and washed with water (20 mL*3) and saturated brine (100 mL*1). The organic layer was dried over Na₂SO₄, filtered and evaporated to afford crude product. The crude product was purified by Prep-TLC with DCM:MeOH=20:1. The residue was purified by Prep-HPLC as following conditions: (Column: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN: Flow rate: 60 mL/min; Gradient: 21% B to 46% B in 8 min: Wave Length: 220/254 nm) to afford the title compound (6.8 mg, yield: 12.8%) as a white solid. LC-MS: (ES, m/z): RT=0.980 min, LCMS: m/z=474.2 [M+1]: 1H NMR (400 MHZ, DMSO-d₆) δ 9.08 (s, 1H), 8.22 (s, 1H), 7.66 (s, 1H), 7.32-7.23 (m, 5H), 7.04 (s, 1H), 4.60-4.45 (m, 1H), 4.08-3.91 (m, 4H), 3.80-3.72 (m, 1H), 3.31 (m, 1H), 2.95-2.87 (m, 1H), 2.22 (s, 3H), 2.17-2.06 (m, 1H), 1.98-1.92 (m, 1H), 1.49 (s, 1H), 1.35-1.21 (m, 1H), 0.92 (q, J=6.6, 6.1 Hz, 2H), 0.76 (s, 2H)

### Example 477: 2-[(1R)-1-{[7-cyclopropoxy-4-(1-methyl-3-phenyl-1H-pyrazol-4-yl)pyrido[3,2-d]pyrimidin-6-yl]oxy}ethyl]-5-methyl-1,3,4-oxadiazole or 2-[(1S)-1-{[7-cyclopropoxy-4-(1-methyl-3-phenyl-1H-pyrazol-4-yl)pyrido[3,2-d]pyrimidin-6-yl]oxy}ethyl]-5-methyl-1,3,4-oxadiazole

### Example 478: 2-[(1S)-1-{[7-cyclopropoxy-4-(1-methyl-3-phenyl-1H-pyrazol-4-yl)pyrido[3,2-d]pyrimidin-6-yl]oxy}ethyl]-5-methyl-1,3,4-oxadiazole or 2-[(1R)-1-{[7-cyclopropoxy-4-(1-methyl-3-phenyl-1H-pyrazol-4-yl)pyrido[3,2-d]pyrimidin-6-yl]oxy}ethyl]-5-methyl-1,3,4-oxadiazole

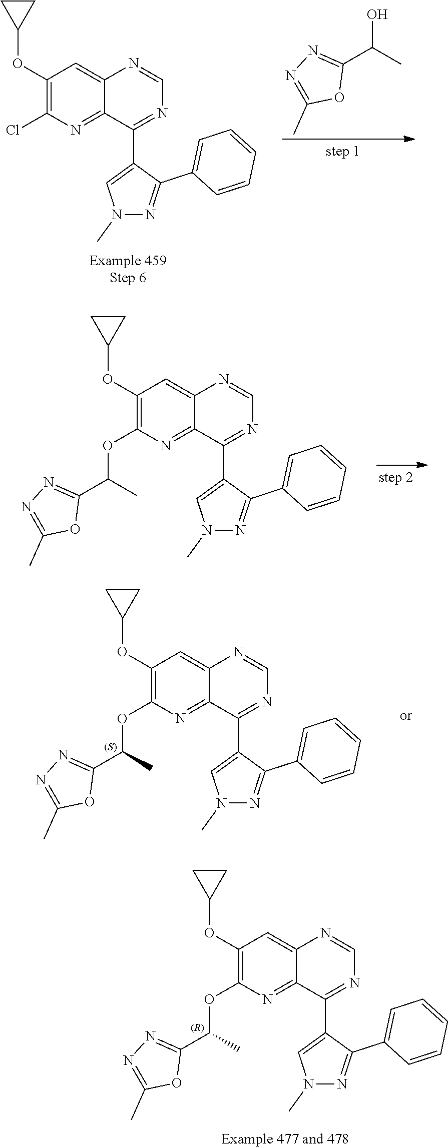

### Step 1: 2-(1-{[7-cyclopropoxy-4-(1-methyl-3-phenyl-1H-pyrazol-4-yl)pyrido[3,2-d]pyrimidin-6-yl]oxy}ethyl)-5-methyl-1,3,4-oxadiazole

t-BuOK (29.5 mg, 264 μmol) was added to 4-{6-chloro-7-cyclopropoxypyrido[3,2-d]pyrimidin-4-yl}-1-methyl-3-phenyl-1H-pyrazole (Product Example 459, Step 6, 50.0 mg, 132 μmol) and 1-(5-methyl-1,3,4-oxadiazol-2-yl)ethan-1-ol (33.8 mg, 264 μmol) in THF (5 mL) at rt. The resulting mixture was heated at 80° C. for 3 h. The reaction mixture was diluted with EtOAc (30 mL), and washed sequentially with water (20 mL). The organic layer was dried over Na₂SO₄, filtered and concentrated under vacuum. The crude product was purified by Prep-TLC with DCM:MeOH=25:1. The residue was purified by Prep-HPLC as following condition: Column: XBridge Prep OBD C18 Column, 30*150 mm, 5 μm; Flow rate: 60 mL/min; Gradient: 35% B to 45% B in 8 min: Wave Length: 254/220 nm: The title compound (56 mg) was collected as a white solid. LC-MS: (ES, m/z): RT=1.004 min, LCMS: m/z=470 [M+1].

### Step 2: Chiral Separation

2-(1-{[7-cyclopropoxy-4-(1-methyl-3-phenyl-1H-pyrazol-4-yl)pyrido[3,2-d]pyrimidin-6-yl]oxy}ethyl)-5-methyl-1,3,4-oxadiazole (56 mg, 119 μmol) in MeOH was purified by Prep-Chiral-HPLC with following condition: Column: Lux 5 μm Cellulose-4, 2.12*25 cm, 5 um; Mobile Phase A: Hex (0.5% 2M NH₃—MeOH)—HPLC, Mobile Phase B: EtOH—HPLC: Flow rate: 20 mL/min; Gradient: 50% B to 50% B in 23 min; Wave Length: 220/254 nm afforded:

Example 477: (First eluting compound, RT1 (min): 15.46, 3 mg) LC-MS: (ES, m/z): RT=1.133 min, LCMS: m/z=512 [M+1]; Chiral-HPLC(ES): RT=2.46 min, ¹H NMR (400 MHZ, DMSO-d₆) δ 8.94 (s, 1H), 8.54 (s, 1H), 7.90 (s, 1H), 7.44-7.37 (m, 2H), 7.27 (q, J=2.9 Hz, 3H), 6.13 (q, J=6.6 Hz, 1H), 4.24 (s, 1H), 4.04 (s, 3H), 2.41 (s, 3H), 1.66 (d, J=6.7 Hz, 3H), 1.23 (s, 1H), 0.95 (d, J=6.1 Hz, 2H), 0.87-0.82 (m, 2H).

Example 478: (Second eluting compound, RT2 (min): 19.486, 5.1 mg) LC-MS: (ES, m/z): RT=1.133 min, LCMS: m/z=512 [M+1]; Chiral-HPLC(ES): RT=3.21 min, ¹H NMR (400 MHZ, DMSO-d₆) δ 8.94 (s, 1H), 8.54 (s, 1H), 7.90 (s, 1H), 7.40 (dt, J=6.3, 3.7 Hz, 2H), 7.27 (p, J=3.9, 3.4 Hz, 3H), 6.13 (q, J=6.7 Hz, 1H), 4.18 (dt, J=6.1, 3.1 Hz, 1H), 4.04 (s, 3H), 2.41 (s, 3H), 1.66 (d, J=6.7 Hz, 3H), 1.23 (s, 1H), 0.95 (d, J=5.9 Hz, 2H), 0.85 (s, 2H).

### Example 479: 7-(1-methyl-1H-pyrazol-4-yl)-4-(3-methyl-5-phenyl-1H-pyrazol-4-yl)quinazoline

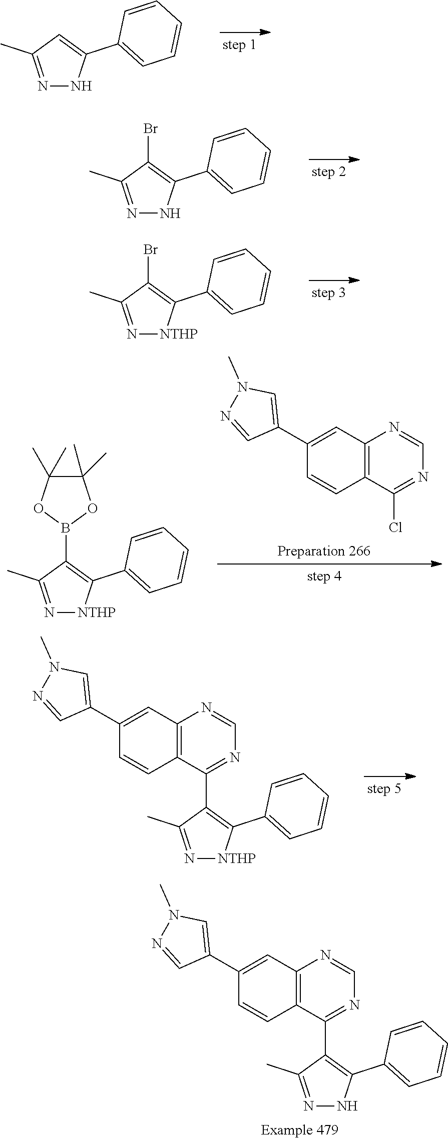

### Step 1: 4-bromo-3-methyl-5-phenyl-1H-pyrazole

To a mixture of 3-methyl-5-phenyl-1H-pyrazole (3 g, 18.9 mmol) in DMF (30 mL) was added N-bromosuccinimide (4.02 g, 22.6 mmol), the reaction mixture was stirred at rt for 3 h. LCMS is ok. The reaction mixture was added to the ice water and extracted with EA (50 mL*3). The organic phase was concentrated under vacuum. The residue was purified by flash chromatography (20% MeOH in DCM) to give the title compound (4 g, yield: 88.8%) as a yellow oil. LC-MS: (ES, m/z): RT=1.075 min, LCMS: m/z=237 [M+1]:

### Step 2: 4-bromo-3-methyl-5-phenyl-1-(tetrahydro-2H-pyran-2-yl)-1H-pyrazole

To a mixture of 4-bromo-3-methyl-5-phenyl-1H-pyrazole (4 g, 16.8 mmol) in DCM (50 mL) was added dihydropyran (7.06 g, 84.0 mmol and para-toluene sulfonate (2.89 g, 16.8 mmol), the reaction mixture was stirred at 60° C. for 4 h. LCMS is ok. The reaction mixture was added to the ice water and extracted with EA (50 mL*3). The organic phase was concentrated under vacuum. The residue was purified by flash chromatography (10% MeOH in DCM) to give the title compound (2.3 g. yield: 42.6%) as a yellow solid. LC-MS: (ES, m/z): RT=2.761 min, LCMS: m/z=278 [M+1]:

### Step 3: 3-methyl-5-phenyl-1-(tetrahydro-2H-pyran-2-yl)-4-(4,4,5,5-tetramethyl-1,3,2-dioxaborolan-2-yl)-1H-pyrazole

To a solution of 4-bromo-3-methyl-1-(oxan-2-yl)-5-phenyl-1H-pyrazole (1.7 g. 5.29 mmol) in dry DMSO (20 mL) was added 4,4,5,5-tetramethyl-2-(4,4,5,5-tetramethyl-1,3,2-dioxaborolan-2-yl)-1,3,2-dioxaborolane (2.66 g, 10.5 mmol), tetrakis(triphenylphosphine) palladium (611 mg, 529 μmol) and potassium acetate (1.03 g. 10.5 mmol) under nitrogen. The mixture was stirred at 120° C. for 6 hrs. LCMS showed the reaction was completed. The reaction mixture was cooled to room temperature. The resulting solution was diluted with 25 ml of water. The resulting solution was extracted with 2×40 mL of ethyl acetate and the organic layers combined. The resulting mixture was washed with 20 mL of brine. The mixture was dried over anhydrous sodium sulfate and concentrated under vacuum. The product was purified by chromatography with DCM:MeOH (20:1). This resulted in (800 mg, yield: 41.2%) the title compound as a light yellow solid. LC-MS: (ES, m/z): RT=1.958 min, LCMS: m/z=369 [M+1]:

### Step 4:7-(1-methyl-1H-pyrazol-4-yl)-4-(3-methyl-5-phenyl-1-(tetrahydro-2H-pyran-2-yl)-1H-pyrazol-4-yl)quinazoline

The reaction mixture of 3-methyl-5-phenyl-1-(tetrahydro-2H-pyran-2-yl)-4-(4,4,5,5-tetramethyl-1,3,2-dioxaborolan-2-yl)-1H-pyrazole 394 mg, 1.07 mmol), 4-chloro-7-(1-methyl-1H-pyrazol-4-yl)quinazoline (Preparation 266, 400 mg, 1.63 mmol), K₃PO₄ (227 mg, 1.63 mmol) and tetrakis(triphenylphosphine) palladium (62.2 mg, 53.9 μmol) in dioxnane/H₂O (20 mL/5 mL) was stirred at 80° C. for 2H. LCMS showed the reaction was completed. The reaction mixture was cooled to rt. The resulting solution was diluted with 25 mL of water. The resulting solution was extracted with 2×40 mL of EA and the organic layers were combined. The resulting mixture was washed with 20 mL of brine. The mixture was dried over anhydrous sodium sulfate and concentrated under vacuum. The product was purified by chromatography with DCM:MeOH (50:1). This resulted in 4-[3-methyl-1-(oxan-2-yl)-5-phenyl-1H-pyrazol-4-yl]-7-(1-methyl-1H-pyrazol-4-yl)quinazoline (200 mg, yield: 82.6%) as an off-white solid. LC-MS: (ES, m/z): RT=1.695 min, LCMS: m/z=451 [M+1]:

### Step 5: 7-(1-methyl-1H-pyrazol-4-yl)-4-(3-methyl-5-phenyl-1H-pyrazol-4-yl)quinazoline

TFA (0.5 mL) was added dropwise to 7-(1-methyl-1H-pyrazol-4-yl)-4-(3-methyl-5-phenyl-1-(tetrahydro-2H-pyran-2-yl)-1H-pyrazol-4-yl)quinazoline (20 mg, 44.3 μmol) in CH₂Cl₂ (2 mL) at rt. The resulting solution was stirred at rt for 2 h. The resulting solution was concentrated under vacuum. the residue was purified by Prep-TLC with DCM/MeOH (20:1). The crude product was purified by Prep-Flash-HPLC with following conditions (Column: YMC-Actus Triart C18, 30*250.5 um; Mobile Phase A: Water (10 mmol/L NH₄HCO₃+0.1% NH₃·H₂O), Mobile Phase B: ACN; Flow rate: 60 mL/min; Gradient: 20% B to 50% B in 7 min: 254,220 nm. This resulted in the title compound (5 mg, yield: 30.7%) as white solid. LC-MS: (ES, m/z): RT=1.072 min, LCMS: m/z=367 [M+1]; ¹H NMR (400 MHZ, DMSO-d₆) δ 9.26 (s, 1H), 8.42 (s, 1H), 8.19-8.10 (m, 2H), 7.72 (dd, J=8.8, 1.8 Hz, 1H), 7.59 (d, J=8.7 Hz, 1H), 7.23 (d, J=4.8 Hz, 3H), 7.18 (s, 2H), 3.89 (s, 3H), 2.54 (s, 3H).

### Example 480: 4-(1,5-dimethyl-3-phenyl-1H-pyrazol-4-yl)-7-(1-methyl-1H-pyrazol-4-yl)quinazoline

### Example 481: 4-(1,3-dimethyl-5-phenyl-1H-pyrazol-4-yl)-7-(1-methyl-1H-pyrazol-4-yl)quinazoline

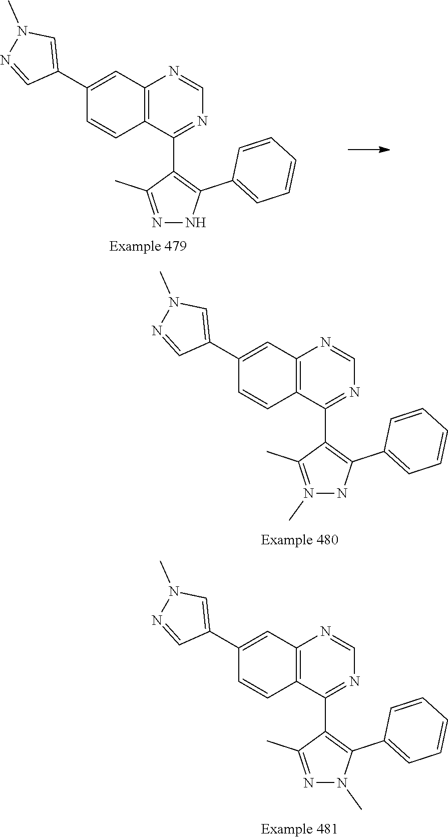

Methyl iodide (69.5 mg, 490 μmol) was added dropwise to 7-(1-methyl-1H-pyrazol-4-yl)-4-(3-methyl-5-phenyl-1H-pyrazol-4-yl)quinazoline (Example 479, 90 mg, 245 μmol) and Cs₂CO₃ (40 mg, 367.5 μmol) in DMF (2 mL) at rt. The resulting solution was stirred at rt for 2 h. The resulting solution was concentrated under vacuum, the residue was purified by Prep-TLC with DCM/MeOH (50:1). The crude product was purified by Prep-Flash-HPLC with following conditions (Column: XBridge Prep OBD C18 Column, 30×150 mm 5 um; Mobile Phase A: Water (10 mmol/L NH₄HCO₃+0.1% NH₃·H₂O), Mobile Phase B: ACN; Flow rate: 60 mL/min; Gradient: 24 B to 39 B in 7 min: 254/220 nm to afford:

Example 481 first eluting peak, 4-(1,3-dimethyl-5-phenyl-1H-pyrazol-4-yl)-7-(1-methyl-1H-pyrazol-4-yl)quinazoline (16 mg, yield: 17.8%) as a white solid. LC-MS: (ES, m/z): RT=1.147 min, LCMS: m/z=381 [M+1]; ¹H NMR (400 MHZ, DMSO-d₆) δ 9.19 (s, 1H), 8.41 (s, 1H), 8.12-8.05 (m, 2H), 7.71-7.57 (m, 2H), 7.34-7.21 (m, 6H), 3.86 (d, J=18.1 Hz, 6H), 2.17 (s, 3H).

Example 480: Second eluting peak, 4-(1,5-dimethyl-3-phenyl-1H-pyrazol-4-yl)-7-(1-methyl-1H-pyrazol-4-yl)quinazoline (20 mg, 22.2%) as a white solid. LC-MS: (ES, m/z): RT=1.156 min, LCMS: m/z=381 [M+1]: 1H NMR (400 MHZ, DMSO-d₆) δ 9.27 (s, 1H), 8.43 (s, 1H), 8.19-8.09 (m, 2H), 7.79-7.56 (m, 2H), 7.24-7.06 (m, 6H), 3.91 (d, J=20.3 Hz, 6H), 2.22 (s, 3H).

### Example 482: 4-(1,5-dimethyl-3-phenyl-1H-pyrazol-4-yl)-7-methoxyquinazolin-6-yl(S)-2,4-dimethylpiperazine-1-carboxylate

Prepared using chemistry analogous to Example 480 and Example 59.

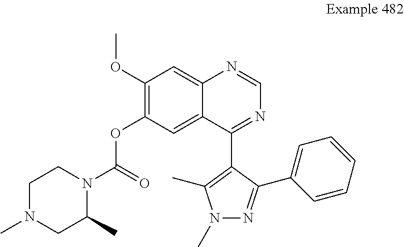 [SMILES: COc1cc2ncnc(-c3c(-c4ccccc4)nn(C)c3C)c2cc1OC(=O)N1CCN(C)C[C@@H]1C]

### Example 483: N-(4-(1-methyl-3-phenyl-1H-pyrazol-4-yl)-7-(trifluoromethyl)pyrido[3,2-d]pyrimidin-6-yl)-3-oxabicyclo[3.1.0]hexane-1-carboxamide

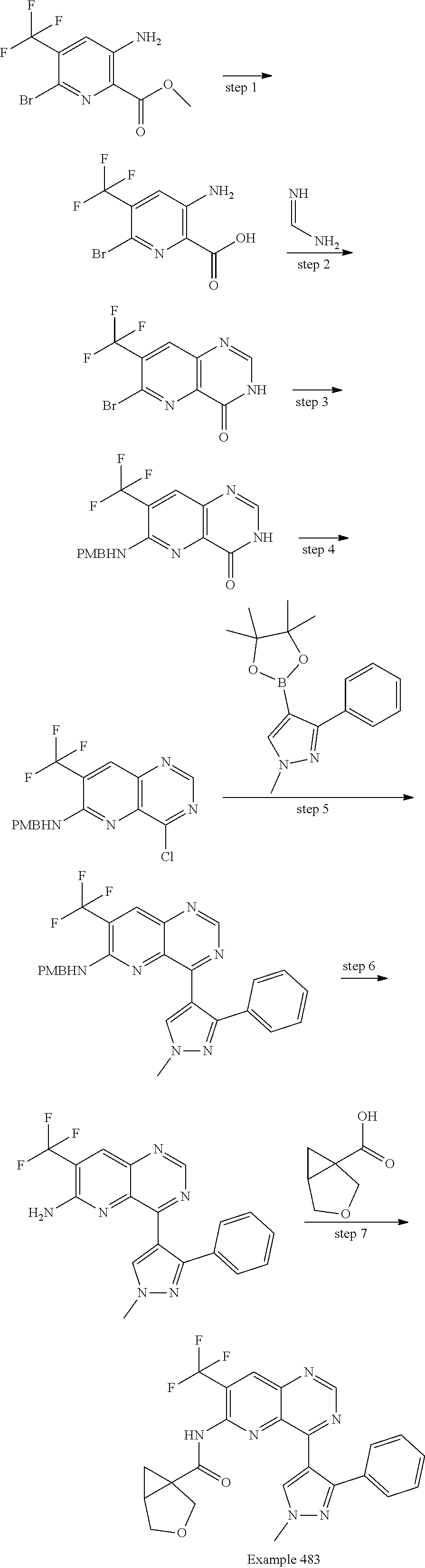

Step 1: Synthesis of 3-amino-6-bromo-5-(trifluoromethyl)pyridine-2-carboxylic acid: LiOH (250 mg, 10.0 mmol) was added to methyl 3-amino-6-bromo-5-(trifluoromethyl)pyridine-2-carboxylate (600 mg, 2.00 mmol) in THF/H₂O (12 mL/3 mL) at 0° C. The reaction mixture was stirred at rt for 2 h. The resulting solution was extracted with 3×100 mL of EA. The organic layer was dried with Na₂SO₄ and concentrated under vacuum. This resulted in the title compound (500 mg, yield: 87%) as a white solid. LC-MS: (ES, m/z): RT=0.697 min, LCMS: m/z=283 [M−1].

Step 2: Synthesis of 6-bromo-7-(trifluoromethyl)-3H,4H-pyrido[3,2-d]pyrimidin-4-one:

The reaction mixture of 3-amino-6-bromo-5-(trifluoromethyl)pyridine-2-carboxylic acid (480 mg, 1.68 mmol) and Formamidine acetate (529 mg, 5.04 mmol) in EtOH (20 mL) was heated at 80° C. for 16 h. The resulting mixture was concentrated under vacuum. The residue was diluted with EA (100 mL) and extracted with 3×100 mL of EA. The organic layer was dried with Na₂SO₄ and concentrated under vacuum. This resulted in the title compound (400 mg, yield: 81%) as a white solid. LC-MS: (ES, m/z): RT=0.533 min, LCMS: m/z=294 [M+1].

Step 3: Synthesis of 6-{[(4-methoxyphenyl)methyl]amino}-7-(trifluoromethyl)-3H,4H-pyrido[3,2-d]pyrimidin-4-one:

The reaction mixture of 6-bromo-7-(trifluoromethyl)-3H,4H-pyrido[3,2-d]pyrimidin-4-one (350 mg, 1.19 mmol) and 1-(4-methoxyphenyl) methanamine (326 mg, 2.38 mmol) in DMSO (6 mL) was heated to 80° C. for 16 h. The resulting solution was extracted with 3×100 mL of EA. The organic layer was dried with Na₂SO₄ and concentrated under vacuum. The crude product was purified by a silica gel column with DCM:MeOH=20:1. This resulted in the title compound (350 mg, yield: 84%) as a yellow solid. LC-MS: (ES, m/z): RT=0.845 min, LCMS: m/z=351 [M+1].

Step 4: Synthesis of 4-chloro-N-[(4-methoxyphenyl)methyl]-7-(trifluoromethyl)pyrido[3,2-d]pyrimidin-6-amine:

N,N-diethylaniline (1.04 g, 6.99 mmol) was added to 6-{[(4-methoxyphenyl)methyl]amino}-7-(trifluoromethyl)-3H,4H-pyrido[3,2-d]pyrimidin-4-one (350 mg, 999 μmol) and POCl₃ (763 mg, 4.99 mmol) in ACN (15 mL) at 0° C. The reaction mixture was heated at 80° C. for 2 h. The resulting solution was added to ice-water, then Na₂CO₃(aq) was adjusted to pH=8. The solution was extracted with EA and concentrated under vacuum. The crude product was purified by a silica gel column with DCM:EA=5:1. This resulted in the title compound (300 mg, yield: 81%) as a yellow solid. LC-MS: (ES, m/z): RT=1.295 min, LCMS: m/z=369 [M+1].

### Step 5: Synthesis of N-[(4-methoxyphenyl)methyl]-4-(1-methyl-3-phenyl-1H-pyrazol-4-yl)-7-(trifluoromethyl)pyrido[3,2-d]pyrimidin-6-amine

The reaction mixture of 4-chloro-N-[(4-methoxyphenyl)methyl]-7-(trifluoromethyl)pyrido[3,2-d]pyrimidin-6-amine (300 mg, 813 μmol), 1-methyl-3-phenyl-4-(4,4,5,5-tetramethyl-1,3,2-dioxaborolan-2-yl)-1H-pyrazole (231 mg, 813 μmol), Pd(PPh₃)₄ (93.9 mg, 81.3 μmol) and K₃PO₄ (257 mg, 1.21 mmol) in dioxane/H₂O (10 mL/2 mL) was heated at 80° C. for 2 h. The resulting solution was extracted with 3×30 mL of EA. The organic layer was dried with Na₂SO₄ and concentrated under vacuum. The crude product was purified by a silica gel column with DCM:MeOH=25:1. This resulted in the title compound (360 mg, yield: 90%) as a yellow solid. LC-MS: (ES, m/z): RT=1.258 min, LCMS: m/z=491 [M+1]

### Step 6:4-(1-methyl-3-phenyl-1H-pyrazol-4-yl)-7-(trifluoromethyl)pyrido[3,2-d]pyrimidin-6-amine

The reaction mixture of N-[(4-methoxyphenyl)methyl]-4-(1-methyl-3-phenyl-1H-pyrazol-4-yl)-7-(trifluoromethyl)pyrido[3,2-d]pyrimidin-6-amine (360 mg, 733 μmol) in TFA (5 mL) was heated at 60° C. for 2 h. The resulting mixture was concentrated under vacuum. The residue was diluted with EA (30 mL) and extracted with 3×30 mL of EA. The organic layer was washed with (sat) NaHCO₃(aq) (50 mL) and dried with Na₂SO₄ and concentrated under vacuum. This resulted in the title compound (210 mg, yield: 77%) as a yellow solid. LC-MS: (ES, m/z): RT=1.115 min, LCMS: m/z=371 [M+1]

Step 7: N-(4-(1-methyl-3-phenyl-1H-pyrazol-4-yl)-7-(trifluoromethyl)pyrido[3,2-d]pyrimidin-6-yl)-3-oxabicyclo[3.1.0]hexane-1-carboxamide:

TEA (329 mg, 3.23 mmol) was added to 3-oxabicyclo[3.1.0]hexane-1-carboxylic acid (206 mg, 1.61 mmol) and 2,4,6-trichlorobenzoyl chloride (392 mg, 1.61 mmol) in THF (8 mL) at 0° C. The reaction mixture was stirred at rt for 1 h. Then 4-(1-methyl-3-phenyl-1H-pyrazol-4-yl)-7-(trifluoromethyl)pyrido[3,2-d]pyrimidin-6-amine (200 mg, 540 μmol) was added in the mixture at 0° C. The reaction mixture was stirred at 80° C. for 16 h. The resulting solution was extracted with 3×60 mL of EA. The organic layer was dried with Na₂SO₄ and concentrated under vacuum. The crude product was purified by Prep-HPLC as following condition: Column: XBridge Shield RP18 OBD Column, 30*150 mm, 5 um; Mobile Phase A: Water (0.1% FA), Mobile Phase B: ACN; Flow rate: 60 mL/min; Gradient: 19% B to 59% B in 7 min: Wave Length: 254/220 nm; This resulted in the title compound (36.0 mg, yield: 13%) as a light yellow solid. LC-MS: (ES, m/z): RT=1.351 min, LCMS: m/z=481 [M+1]; ¹H NMR (300 MHz, DMSO-d₆) δ 10.13 (s, 1H), 9.17 (s, 1H), 8.87 (d, J=19.3 Hz, 2H), 7.57-7.46 (m, 2H), 7.38-7.28 (m, 3H), 4.07-3.93 (m, 5H), 3.86-3.72 (m, 2H), 2.31 (d, J=6.2 Hz, 1H), 1.52 (dd, J=8.3, 4.4 Hz, 1H), 1.00 (t, J=5.0 Hz, 1H).

### Example 484: N-(7-methoxy-4-(3-phenyl-1-(trifluoromethyl)-1H-pyrazol-4-yl)quinazolin-6-yl)bicyclo[1.1.1]pentane-1-carboxamide

### Example 485: N-(7-methoxy-4-(5-phenyl-1-(trifluoromethyl)-1H-pyrazol-4-yl)quinazolin-6-yl)bicyclo[1.1.1]pentane-1-carboxamide

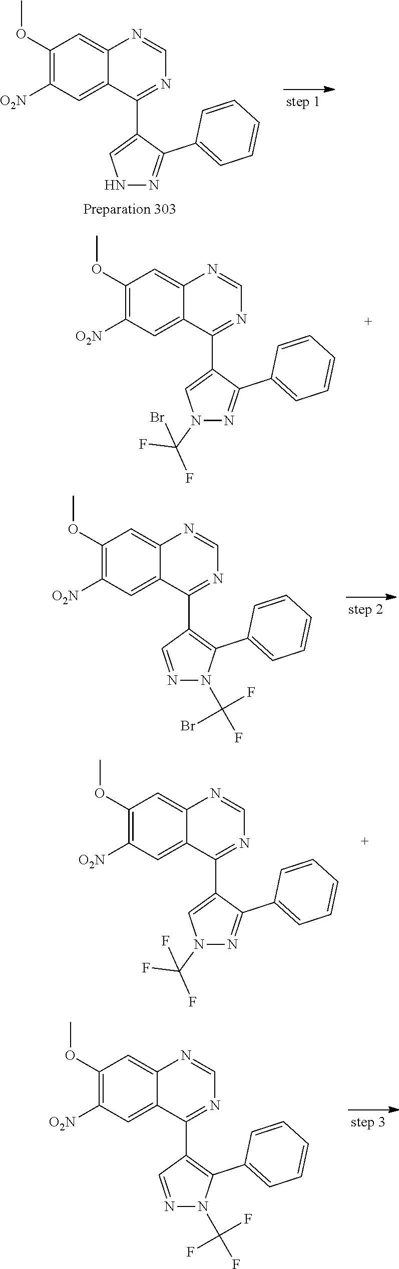 [SMILES: COc1cc2ncnc(-c3c[nH]nc3-c3ccccc3)c2cc1[N+](=O)[O-] ; COc1cc2ncnc(-c3cn(C(F)(F)Br)nc3-c3ccccc3)c2cc1[N+](=O)[O-] ; COc1cc2ncnc(-c3cnn(C(F)(F)Br)c3-c3ccccc3)c2cc1[N+](=O)[O-] ; COc1cc2ncnc(-c3cn(C(F)(F)F)nc3-c3ccccc3)c2cc1[N+](=O)[O-] ; COc1cc2ncnc(-c3cnn(C(F)(F)F)c3-c3ccccc3)c2cc1[N+](=O)[O-]]

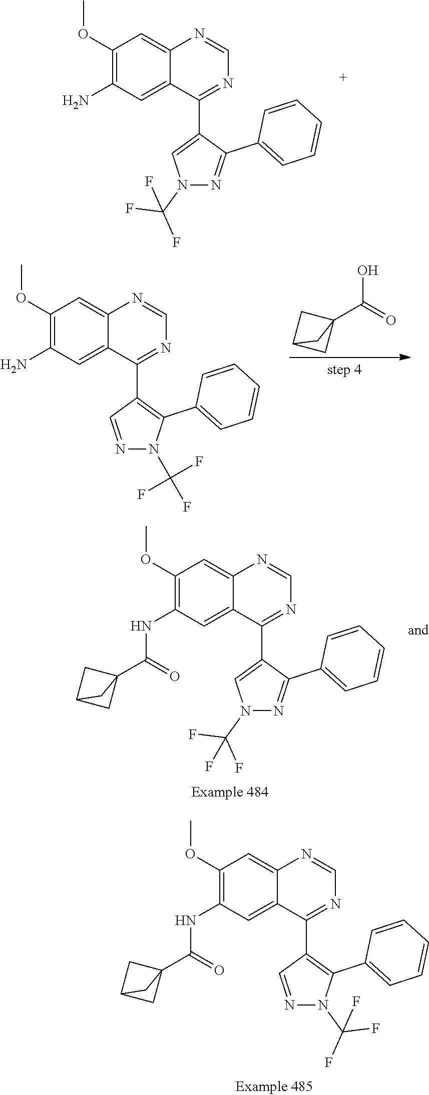

### Step 1: Synthesis mixture of 4-[1-(bromodifluoromethyl)-3-phenyl-1H-pyrazol-4-yl]-7-methoxy-6-nitroquinazoline and 4-[1-(bromodifluoromethyl)-5-phenyl-1H-pyrazol-4-yl]-7-methoxy-6-nitroquinazoline

NaH (92.0 mg, 2.30 mmol) was added to (Preparation 303, 400 mg, 1.15 mmol) in DMF (10 mL) at 0° C. The mixture was stirred at 0° C. for 30 min, then added CF₂Br₂ (478 mg, 2.30 mmol) at 0° C. The resulting mixture was hold to room temperature for 16 h. The reaction mixture was diluted with EA (100 mL), and washed with water (100 mL*3) and saturated brine (100 mL*1). The organic layer was dried over Na₂SO₄, filtered and evaporated to afford crude product. The crude product was purified by Pre-TLC with DCM:MeOH=25:1, to afford the title compound (245 mg, yield: 44.7%) as a yellow solid. LC-MS: (ES, m/z): RT=1.454 min, LCMS: m/z=476 [M+1],

### Step 2: Synthesis mixture of 7-methoxy-6-nitro-4-[3-phenyl-1-(trifluoromethyl)-1H-pyrazol-4-yl]quinazoline and 7-methoxy-6-nitro-4-[5-phenyl-1-(trifluoromethyl)-1H-pyrazol-4-yl]quinazoline

AgBF₄ (198 mg, 1.02 mmol) was added to mixture of 4-[1-(bromodifluoromethyl)-3-phenyl-1H-pyrazol-4-yl]-7-methoxy-6-nitroquinazoline and 4-[1-(bromodifluoromethyl)-5-phenyl-1H-pyrazol-4-yl]-7-methoxy-6-nitroquinazoline (244 mg, 514 μmol) in DCM (10 mL) at −78° C. under N₂. The resulting mixture was hold to room temperature for 16 h. The solid was filtered out. The filtrate was concentrated under vacuum. The crude product was purified by Pre-TLC with DCM:MeOH=20:1, to afford the title compound (150 mg, yield: 70.4%) as an off-white solid. LC-MS: (ES, m/z): RT=1.404 min, LCMS: m/z=416 [M+1],

### Step 3: Synthesis mixture of 7-methoxy-4-[3-phenyl-1-(trifluoromethyl)-1H-pyrazol-4-yl]quinazolin-6-amine and 7-methoxy-4-[5-phenyl-1-(trifluoromethyl)-1H-pyrazol-4-yl]quinazolin-6-amine

Fe (161 mg, 2.88 mmol) and NH₄Cl (155 mg, 2.88 mmol) was added to mixture of 7-methoxy-6-nitro-4-[3-phenyl-1-(trifluoromethyl)-1H-pyrazol-4-yl]quinazoline and 7-methoxy-6-nitro-4-[3-phenyl-1-(trifluoromethyl)-1H-pyrazol-4-yl]quinazoline (150 mg, 361 μmol) in EtOH/H₂O (10 mL/2.5 mL) at rt. The resulting mixture was heated to 80° C. for 2 h. The solid was filtered out. The filtrate was concentrated under vacuum. The crude product was purified by a silica gel column with DCM:MeOH=20:1, to afford the title compound (120 mg, yield: 86.3%) as a white solid. LC-MS: (ES, m/z): RT=1.307 min and 0.902 min, LCMS: m/z=386 [M+1].

### Step 4: Synthesis of N-{7-methoxy-4-[3-phenyl-1-(trifluoromethyl)-1H-pyrazol-4-yl]quinazolin-6-yl}bicyclo[1.1.1]pentane-1-carboxamide and [[PH-BPM-B3116-100, X674333]] N-{7-methoxy-4-[5-phenyl-1-(trifluoromethyl)-1H-pyrazol-4-yl]quinazolin-6-yl}bicyclo[1.1.1]pentane-1-carboxamide

T3P (2 mL) and Py (2 mL) were added to 7-methoxy-4-[3-phenyl-1-(trifluoromethyl)-1H-pyrazol-4-yl]quinazolin-6-amine (120 mg, 311 μmol) and bicyclo[1.1.1]pentane-1-carboxylic acid (69.7 mg. 622 μmol) in THF (5 mL) at rt. The resulting mixture was heated to 60° C. for 2 h. The mixture was concentrated under vacuum. The crude product was purified by Pre-TLC with DCM:MeOH=20:1. The residue was purified by Prep-HPLC as following condition: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN: Flow rate: 60 mL/min; Gradient: 29% B to 70% B in 9 min, 70% B: Wave Length: 254/220 nm. This resulted in 80 mg mixture of the title compounds

The mixture was dissolved in MeOH and was Purified by Prep-Chiral-HPLC with following condition: Column: CHIRAL ART Cellulose-SC. 2*25 cm, 5 μm; Mobile Phase A: Hex (0.5% 2M NH₃—MeOH)—HPLC, Mobile Phase B: IPA—HPLC: Flow rate: 20 mL/min; Gradient: 15% B to 15% B in 27 min: Wave Length: 220/254 nm: RT1 (min): 16.656: RT2 (min): 22.239; Sample Solvent: EtOH-HPLC to afford:

Example 484: First eluting peak (33.3 mg) LC-MS: (ES, m/z): RT=1.192 min, LCMS: m/z=480 [M+1]; 1H NMR (400 MHZ, DMSO-d₆) δ 9.15 (s, 1H), 9.07 (s, 1H), 8.74 (s, 1H), 8.57 (s, 1H), 7.54 (s, 1H), 7.38-7.23 (m, 5H), 4.10 (s, 3H), 2.47 (s, 1H), 2.09 (s, 6H).

Example 484: Second eluting peak (14.8 mg) LC-MS: (ES, m/z): RT=1.213 min, LCMS: m/z=480 [M+1]; 1H NMR (300 MHz, DMSO-d₆) δ 9.02 (s, 1H), 8.75 (s, 1H), 8.60 (s, 1H), 8.39 (s, 1H), 7.47-7.35 (m, 6H), 4.06 (s, 3H), 2.54 (s, 1H), 2.14 (s, 6H).

Example 486: N-(7-(methoxy-d3)-4-(1-(methyl-d3)-3-phenyl-1H-pyrazol-4-yl)pyrido[3,2-d]pyrimidin-6-yl)-1-(trifluoromethyl)cyclopropane-1-carboxamide

Example 487: N-(7-(methoxy-d3)-4-(1-(methyl-d3)-5-phenyl-1H-pyrazol-4-yl)pyrido[3,2-d]pyrimidin-6-yl)-1-(trifluoromethyl)cyclopropane-1-carboxamide

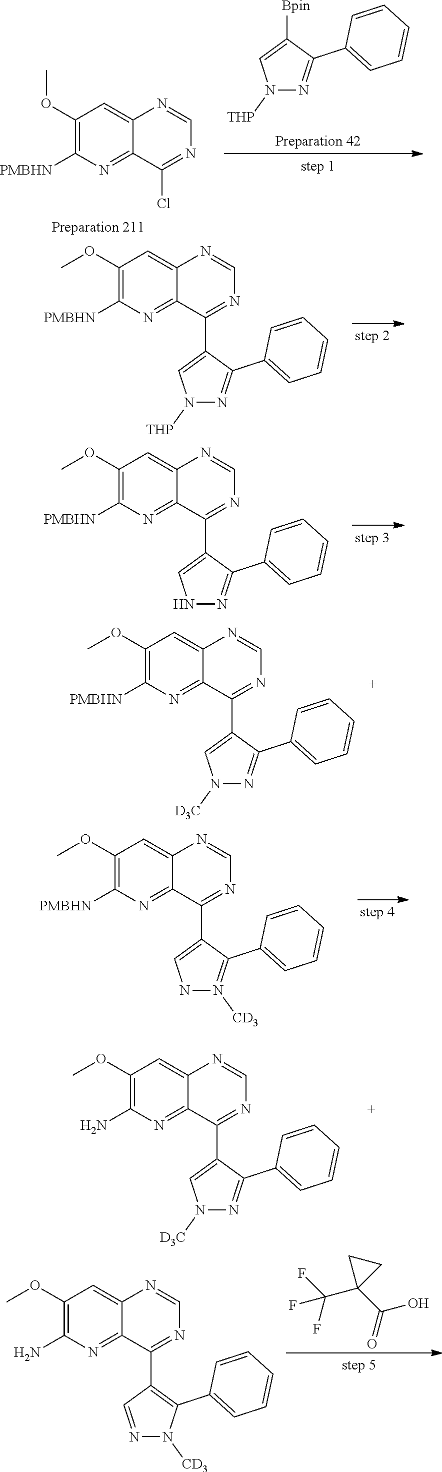

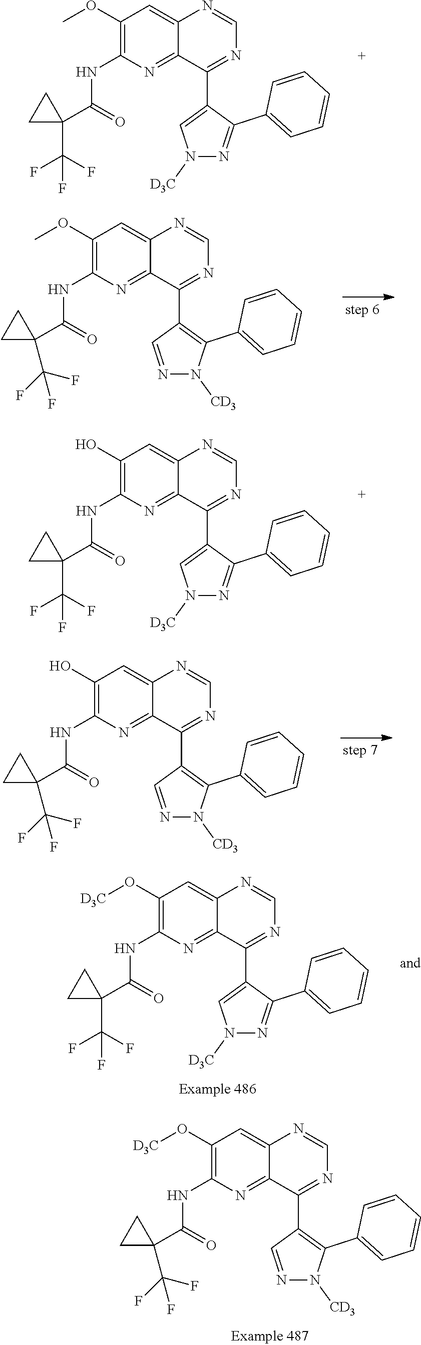

### Step 1: Synthesis of 7-methoxy-N-[(4-methoxyphenyl)methyl]-4-[1-(oxan-2-yl)-3-phenyl-1H-pyrazol-4-yl]pyrido[3,2-d]pyrimidin-6-amine

The reaction mixture of 4-chloro-7-methoxy-N-[(4-methoxyphenyl)methyl]pyrido[3,2-d]pyrimidin-6-amine (Preparation 211, 3 g, 9.06 mmol), 3-phenyl-1-(tetrahydro-2H-pyran-2-yl)-4-(4,4,5,5-tetramethyl-1,3,2-dioxaborolan-2-yl)-1H-pyrazole (Preparation 42, 2.5 g, 9.06 mmol), Pd(dppf)Cl₂ (739 mg, 906 μmol) and K₂CO₃ (1.25 g, 9.06 mmol) in H₂O (9 mL) and dioxane (36 mL) at rt. The resulting mixture was stirred at 80° C. for 2 h under N₂. The mixture was diluted with DCM 200 mL and washed with water 100 mL*2, the organic layer was dried with Na₂SO₄ and concentrated under vacuum. The residue was purified by a silica gel column with DCM:MeOH=20:1. This resulted in the title compound (2.8 g, yield: 59%) as a white solid. LC-MS: (ES, m/z): RT=1.052 min, LCMS: m/z=523 [M+1].

### Step 2: Synthesis of 7-methoxy-N-[(4-methoxyphenyl)methyl]-4-[1-(²H₃)methyl-3-phenyl-1H-pyrazol-4-yl]pyrido[3,2-d]pyrimidin-6-amine

TFA (7 mL) was added to 7-methoxy-N-[(4-methoxyphenyl)methyl]-4-[1-(oxan-2-yl)-3-phenyl-1H-pyrazol-4-yl]pyrido[3,2-d]pyrimidin-6-amine (2.8 g, 5.35 mmol) in DCM (20 mL) at r.t. The resulting mixture was stirred at r.t for 1 h. The mixture was diluted with DCM 200 mL and washed with Sodium bicarbonate aqueous solution 100 mL*2, the organic layer was dried with Na₂SO₄ and concentrated under vacuum. The residue was purified by a silica gel column with DCM:EA=25:1. This resulted in the title compound (2.0 g, yield: 55%) as a white solid. LC-MS: (ES, m/z): RT=1.126 min, LCMS: m/z=439 [M+1].

### Step 3: Synthesis mixture of 7-methoxy-N-[(4-methoxyphenyl)methyl]-4-[1-(²H₃)methyl-3-phenyl-1H-pyrazol-4-yl]pyrido[3,2-d]pyrimidin-6-amine and 7-methoxy-N-(4-methoxybenzyl)-4-(1-(methyl-d3)-5-phenyl-1H-pyrazol-4-yl)pyrido[3,2-d]pyrimidin-6-amine

DIAD (1.61 g, 8.55 mmol) was added to the solution of 7-methoxy-N-[(4-methoxyphenyl)methyl]-4-(3-phenyl-1H-pyrazol-4-yl)pyrido[3,2-d]pyrimidin-6-amine (2.5 g, 5.70 mmol), PPh₃ (2.78 g, 8.55 mmol) and CD₃OD (342 mg, 5.70 mmol) in THF (20 mL) at ice bar under N₂. The resulting mixture was stirred at r.t for 10 h under N₂. The mixture was diluted with EA 200 mL and washed with brine 70 mL*2, the organic layer was dried with Na₂SO₄ and concentrated under vacuum. The residue was purified by a silica gel column with DCM:EA=15:1. This resulted in the title compound (1.5 g, yield: 75%) as a light yellow solid. LC-MS: (ES, m/z): RT=0.897 min, LCMS: m/z=456 [M+1].

### Step 4: Synthesis mixture of 7-methoxy-4-[1-(²H₃)methyl-3-phenyl-1H-pyrazol-4-yl]pyrido[3,2-d]pyrimidin-6-amine and 7-methoxy-4-(1-(methyl-d3)-5-phenyl-1H-pyrazol-4-yl)pyrido[3,2-d]pyrimidin-6-amine

7-methoxy-N-[(4-methoxyphenyl)methyl]-4-[1-(²H₃)methyl-3-phenyl-1H-pyrazol-4-yl]pyrido[3,2-d]pyrimidin-6-amine (2 g, 4.39 mmol) was added to TFA (20 mL) at r.t. The resulting mixture was stirred at 60° C. for 3 h. The mixture was diluted with EA (150 mL) and washed with brine (70 mL*2), the organic layer was dried with Na₂SO₄ and concentrated under vacuum. The residue was purified by a silica gel column with DCM:MeOH=18:1. This resulted in the title compound (1.2 g, yield: 68%) as a white solid. LC-MS: (ES, m/z): RT=0.679 min, LCMS: m/z=336 [M+1].

### Step 5: Synthesis mixture of N-{7-hydroxy-4-[1-(²H₃)methyl-3-phenyl-1H-pyrazol-4-yl]pyrido[3,2-d]pyrimidin-6-yl}-1-(trifluoromethyl)cyclopropane-1-carboxamide and N-(7-methoxy-4-(1-(methyl-d3)-5-phenyl-1H-pyrazol-4-yl)pyrido[3,2-d]pyrimidin-6-yl)-1-(trifluoromethyl)cyclopropane-1-carboxamide

2,4,6-trichlorobenzoyl chloride (1.45 g, 5.96 mmol) and TEA (1.23 g, 8.94 mmol) were added to 1-(trifluoromethyl)cyclopropane-1-carboxylic acid (918 mg, 5.96 mmol) in THF (20 mL) at 0° C. for 1 h. Then 4-[3-(2,4-difluorophenyl)-1-methyl-1H-pyrazol-4-yl]-7-methoxypyrido[3,2-d]pyrimidin-6-amine (1.2 g, 4.87 mmol) added to the mixture at rt. The resulting mixture was stirred at 80° C. for 4 h under N₂. The mixture was diluted with EA 150 mL and washed with brine 70 mL*2, the organic layer was dried with Na₂SO₄ and concentrated under vacuum. The residue was purified by a silica gel column with PE:EA=15:1. This result in the title compound (1 g, yield: 71.4%) as an off-white solid. LC-MS: (ES, m/z): RT=1.234 min, LCMS: m/z=472 [M+1].

### Step 6: Synthesis mixture of N-{7-hydroxy-4-[1-(²H₃)methyl-3-phenyl-1H-pyrazol-4-yl]pyrido[3,2-d]pyrimidin-6-yl}-1-(trifluoromethyl)cyclopropane-1-carboxamide and N-(7-hydroxy-4-(1-(methyl-d3)-5-phenyl-1H-pyrazol-4-yl)pyrido[3,2-d]pyrimidin-6-yl)-1-(trifluoromethyl)cyclopropane-1-carboxamide

L-Selectride (339 mg, 1.06 mmol) was added to N-{7-methoxy-4-[1-(²H₃)methyl-3-phenyl-1H-pyrazol-4-yl]pyrido[3,2-d]pyrimidin-6-yl}-1-(trifluoromethyl)cyclopropane-1-carboxamide (500 mg, 1.06 mmol) in THF (10 mL) at r.t. The resulting mixture was stirred at 80° C. for 2 h. The mixture was concentrated under vacuum. The residue was purified by a silica gel column with DCM:EA=25:1. This resulted in the title compound (150 mg, yield: 31%) as a white solid. LC-MS: (ES, m/z): RT=1.116 min, LCMS: m/z=458 [M+1].

### Step 7: Synthesis of Example 486 and 487

DIAD (83.5 mg, 261 μmol) was added dropwise to the solution of N-{7-hydroxy-4-[1-(²H₃)methyl-3-phenyl-1H-pyrazol-4-yl]pyrido[3,2-d]pyrimidin-6-yl}-1-(trifluoromethyl)cyclopropane-1-carboxamide (80 mg, 174 μmol) and PPh₃ (31.3 mg, 522 μmol) and CD₃OD (31.3 mg, 522 μmol) in THF (10 mL) at ice bar under N₂. The resulting mixture was stirred at r.t for 10 h under N₂. The mixture was diluted with EA 100 mL and washed with brine 50 mL*2, the organic layer was dried with Na₂SO₄ and concentrated under vacuum. The residue was purified by a silica gel column with DCM:EA=15:1. The residue was purified by Column: Xselect CSH F-Phenyl OBD column, 19*250 mm, 5 um; Mobile Phase A: Water (0.1% FA), Mobile Phase B: MeOH—HPLC: Flow rate: 25 mL/min; Gradient: 66% B to 77% B in 10 min: Wave Length: 254 220 nm to afford:

Example 486: first eluting peak (9.9 mg) LC-MS (ES, m/z): RT=1.313 min, LCMS: m/z=475 [M+1]. ¹H NMR (400 MHZ, DMSO-d₆) δ 9.77 (s, 1H), 9.17 (s, 1H), 8.88 (s, 1H), 7.77 (s, 1H), 7.54-7.46 (m, 2H), 7.32 (dd, J=5.1, 2.0 Hz, 3H), 1.62 (s, 2H), 1.53-1.42 (m, 2H).

Example 487: second eluting peak (5.3 mg) LC-MS: (ES, m/z): RT=1.288 min, LCMS: m/z=475 [M+1]; ¹H NMR (400 MHZ, DMSO-d₆) δ 9.74 (s, 1H), 8.96 (s, 1H), 8.73 (s, 1H), 7.71 (s, 1H), 7.49-7.36 (m, 5H), 1.65 (s, 2H), 1.38 (s, 2H).

### Example 488: N-(4-(1-methyl-3-phenyl-1H-pyrazol-4-yl)-7-(trifluoromethyl)pyrido[3,2-d]pyrimidin-6-yl)-3-oxabicyclo[3.1.0]hexane-1-carboxamide

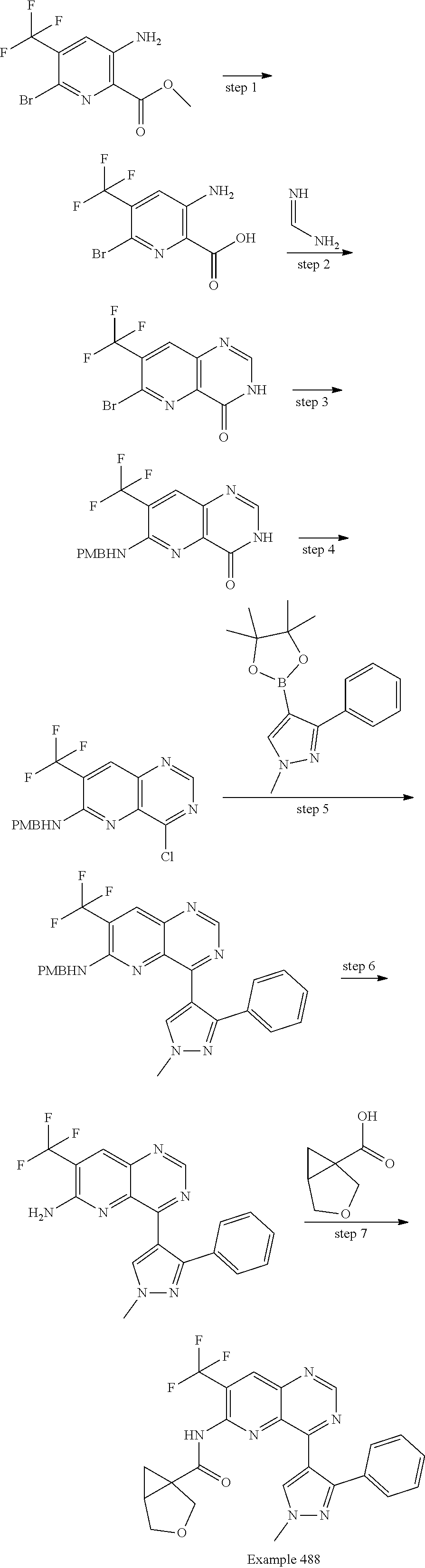

### Step 1: Synthesis of 3-amino-6-bromo-5-(trifluoromethyl)pyridine-2-carboxylic Acid

LiOH (250 mg, 10.0 mmol) was added to methyl 3-amino-6-bromo-5-(trifluoromethyl)pyridine-2-carboxylate (600 mg, 2.00 mmol) in THF/H₂O (12 mL/3 mL) at 0° C. The reaction mixture was stirred at rt for 2 h. The resulting solution was extracted with 3×100 mL of EA. The organic layer was dried with Na₂SO₄ and concentrated under vacuum. This resulted in the title compound (500 mg, yield: 87%) as a white solid. LC-MS: (ES, m/z): RT=0.697 min, LCMS: m/z=283 [M−1].

### Step 2: Synthesis of 6-bromo-7-(trifluoromethyl)-3H,4H-pyrido[3,2-d]pyrimidin-4-one

The reaction mixture of 3-amino-6-bromo-5-(trifluoromethyl)pyridine-2-carboxylic acid (480 mg, 1.68 mmol) and Formamidine acetate (529 mg, 5.04 mmol) in EtOH (20 mL) was heated at 80° C. for 16 h. The resulting mixture was concentrated under vacuum. The residue was diluted with EA (100 mL) and extracted with 3×100 mL of EA. The organic layer was dried with Na₂SO₄ and concentrated under vacuum. This resulted in the title compound (400 mg, yield: 81%) as a white solid. LC-MS: (ES, m/z): RT=0.533 min, LCMS: m/z=294 [M+1].

### Step 3: Synthesis of 6-{[(4-methoxyphenyl)methyl]amino}-7-(trifluoromethyl)-3H,4H-pyrido[3,2-d]pyrimidin-4-one

The reaction mixture of 6-bromo-7-(trifluoromethyl)-3H,4H-pyrido[3,2-d]pyrimidin-4-one (350 mg, 1.19 mmol) and 1-(4-methoxyphenyl) methanamine (326 mg, 2.38 mmol) in DMSO (6 mL) was heated to 80° C. for 16 h. The resulting solution was extracted with 3×100 mL of EA. The organic layer was dried with Na₂SO₄ and concentrated under vacuum. The crude product was purified by a silica gel column with DCM:MeOH=20:1. This resulted in the title compound (350 mg, yield: 84%) as a yellow solid. LC-MS: (ES, m/z): RT=0.845 min, LCMS: m/z=351 [M+1].

### Step 4: Synthesis of 4-chloro-N-[(4-methoxyphenyl)methyl]-7-(trifluoromethyl)pyrido[3,2-d]pyrimidin-6-amine

N,N-diethylaniline (1.04 g. 6.99 mmol) was added to 6-{[(4-methoxyphenyl)methyl]amino}-7-(trifluoromethyl)-3H,4H-pyrido[3,2-d]pyrimidin-4-one (350 mg, 999 μmol) and POCl₃ (763 mg, 4.99 mmol) in ACN (15 mL) at 0° C. The reaction mixture was heated at 80° C. for 2 h. The resulting solution was added to ice-water, then Na₂CO₃ (aq) was adjusted to pH=8. The solution was extracted with EA and concentrated under vacuum. The crude product was purified by a silica gel column with DCM:EA=5:1. This resulted in the title compound (300 mg, yield: 81%) as a yellow solid. LC-MS: (ES, m/z): RT=1.295 min, LCMS: m/z=369 [M+1].

### Step 5: Synthesis of N-[(4-methoxyphenyl)methyl]-4-(1-methyl-3-phenyl-1H-pyrazol-4-yl)-7-(trifluoromethyl)pyrido[3,2-d]pyrimidin-6-amine

The reaction mixture of 4-chloro-N-[(4-methoxyphenyl)methyl]-7-(trifluoromethyl)pyrido[3,2-d]pyrimidin-6-amine (300 mg, 813 μmol), 1-methyl-3-phenyl-4-(4,4,5,5-tetramethyl-1,3,2-dioxaborolan-2-yl)-1H-pyrazole (231 mg, 813 μmol), Pd(PPh₃)₄ (93.9 mg, 81.3 μmol) and K₃PO₄ (257 mg, 1.21 mmol) in dioxane/H₂O (10 mL/2 mL) was heated at 80° C. for 2 h. The resulting solution was extracted with 3×30 mL of EA. The organic layer was dried with Na₂SO₄ and concentrated under vacuum. The crude product was purified by a silica gel column with DCM:MeOH=25:1. This resulted in the title compound (360 mg, yield: 90%) as a yellow solid. LC-MS: (ES, m/z): RT=1.258 min, LCMS: m/z=491 [M+1]

### Step 6: Synthesis of 4-(1-methyl-3-phenyl-1H-pyrazol-4-yl)-7-(trifluoromethyl)pyrido[3,2-d]pyrimidin-6-amine

The reaction mixture of N-[(4-methoxyphenyl)methyl]-4-(1-methyl-3-phenyl-1H-pyrazol-4-yl)-7-(trifluoromethyl)pyrido[3,2-d]pyrimidin-6-amine (360 mg, 733 μmol) in TFA (5 mL) was heated at 60° C. for 2 h. The resulting mixture was concentrated under vacuum. The residue was diluted with EA (30 mL) and extracted with 3×30 mL of EA. The organic layer was washed with (sat) NaHCO₃(aq) (50 mL) and dried with Na₂SO₄ and concentrated under vacuum. This resulted in the title compound (210 mg, yield: 77%) as a yellow solid. LC-MS: (ES, m/z): RT=1.115 min, LCMS: m/z=371 [M+1]

### Step 7: Synthesis of [[PH-BPM-B3185-0,]] N-(4-(1-methyl-3-phenyl-1H-pyrazol-4-yl)-7-(trifluoromethyl)pyrido[3,2-d]pyrimidin-6-yl)-3-oxabicyclo[3.1.0]hexane-1-carboxamide

TEA (329 mg, 3.23 mmol) was added to 3-oxabicyclo[3.1.0]hexane-1-carboxylic acid (206 mg, 1.61 mmol) and 2,4,6-trichlorobenzoyl chloride (392 mg, 1.61 mmol) in THF (8 mL) at 0° C. The reaction mixture was stirred at rt for 1 h. Then 4-(1-methyl-3-phenyl-1H-pyrazol-4-yl)-7-(trifluoromethyl)pyrido[3,2-d]pyrimidin-6-amine (200 mg, 540 μmol) was added in the mixture at 0° C. The reaction mixture was stirred at 80° C. for 16 h. The resulting solution was extracted with 3×60 mL of EA. The organic layer was dried with Na₂SO₄ and concentrated under vacuum. The crude product was purified by Prep-HPLC as following condition: Column: XBridge Shield RP18 OBD Column, 30*150 mm, 5 um; Mobile Phase A: Water (0.1% FA), Mobile Phase B: ACN; Flow rate: 60 mL/min; Gradient: 19% B to 59% B in 7 min; Wave Length: 254/220 nm; This resulted in the title compound (36.0 mg, yield: 13%) as a light yellow solid. LC-MS: (ES, m/z): RT=1.351 min, LCMS: m/z=481 [M+1]; 1H NMR (300 MHz, DMSO-d₆) δ 10.13 (s, 1H), 9.17 (s, 1H), 8.87 (d, J-19.3 Hz, 2H), 7.57-7.46 (m, 2H), 7.38-7.28 (m, 3H), 4.07-3.93 (m, 5H), 3.86-3.72 (m, 2H), 2.31 (d, J=6.2 Hz, 1H), 1.52 (dd, J=8.3, 4.4 Hz, 1H), 1.00 (t, J=5.0 Hz, 1H).

The following compounds were prepared using the appropriate starting materials and the methods described above.

| Example | Structure | Data |
|---|---|---|
| 489 | 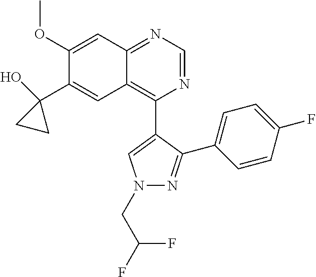 [SMILES: COc1cc2ncnc(-c3cn(CC(F)F)nc3-c3ccc(F)cc3)c2cc1C1(O)CC1] | LC-MS: (ES, m/z): RT = 0.945 min, LCMS: m/z = 441 [M + 1]; Chiral- HPLC RT = 1.549 min. ¹H NMR (300 MHz, DMSO-d₆) δ 9.12 (s, 1H), 8.36 (s, 1H), 7.72 (s, 1H), 7.41-7.30 (m, 3H), 7.08 (t, J = 8.9 Hz, 2H), 6.87-6.09 (m, 1H), 5.55 (s, 1H), 4.84 (td, J = 15.0, 3.7 Hz, 2H), 4.00 (s, 3H), 0.90-0.80 (m, 2H), 0.64-0.54 (m, 2H). |
| 490 | 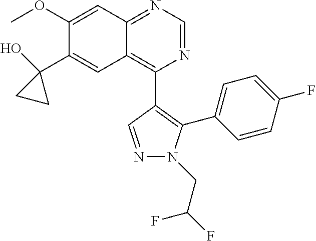 [SMILES: COc1cc2ncnc(-c3cnn(CC(F)F)c3-c3ccc(F)cc3)c2cc1C1(O)CC1] | LC-MS: (ES, m/z): RT = 1.770 min, LCMS: m/z = 441 [M + 1]; Chiral- HPLC RT = 2.018 min. ¹H NMR (400 MHz, DMSO-d₆) δ 8.99 (s, 1H), 8.14 (s, 1H), 7.89 (s, 1H), 7.44-7.33 (m, 2H), 7.31 (s, 1H), 7.28-7.17 (m, 2H), 6.46 (tt, J = 54.7, 3.8 Hz, 1H), 5.65 (s, 1H), 4.61 (td, J = 14.6, 3.8 Hz, 2H), 3.97 (s, 3H), 0.94 (t, J = 2.7 Hz, 2H), 0.93-0.83 (m, 2H). |
| 492 | 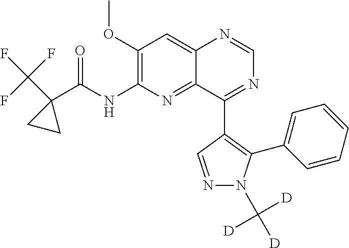 [SMILES: [2H]C([2H])([2H])n1ncc(-c2ncnc3cc(OC)c(NC(=O)C4(C(F)(F)F)CC4)nc23)c1-c1ccccc1] | LC-MS: (ES, m/z): RT = 1.197 min, LCMS: m/z = 472 [M + 1], ¹H NMR (400 MHz, DMSO-d₆) δ 9.74 (s, 1H), 8.96 (s, 1H), 8.74 (s, 1H), 7.72 (s, 1H), 7.50- 7.42 (m, 3H), 7.44-7.36 (m, 2H), 4.06 (s, 3H), 1.60 (s, 2H), 1.55-1.43 (m, 2H), 1.23 (s, 1H). |
| | Additional Product of | |
| | Example 320 | |
| 493 | 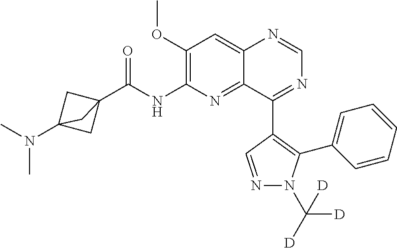 [SMILES: [2H]C([2H])([2H])n1ncc(-c2ncnc3cc(OC)c(NC(=O)C45CC(N(C)C)(C4)C5)nc23)c1-c1ccccc1] | LC-MS: (ES, m/z): RT = 1.400 min, LCMS: m/z = 473 [M + 1]; ¹H NMR (400 MHz, DMSO-d₆) δ 9.59 (s, 1H), 9.13 (s, 1H), 8.69 (s, 1H), 7.68 (s, 1H), 7.50-7.43 (m, 3H), 7.43-7.37 (m, 2H), 4.06 (s, 3H), 2.16 (s, 6H), 2.08 (s, 6H). |
| 494 | 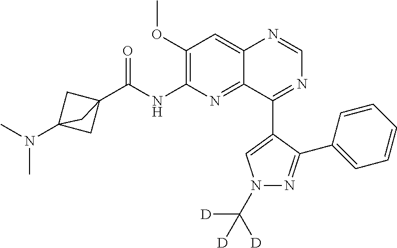 [SMILES: [2H]C([2H])([2H])n1cc(-c2ncnc3cc(OC)c(NC(=O)C45CC(N(C)C)(C4)C5)nc23)c(-c2ccccc2)n1] | LC-MS: (ES, m/z): RT = 1.095 min, LCMS: m/z = 473 [M + 1]; ¹H NMR (400 MHz, DMSO-d₆) δ 9.56 (s, 1H), 9.42 (s, 1H), 8.83 (s, 1H), 7.73 (s, 1H), 7.55-7.49 (m, 2H), 7.33 (dd, J = 5.1, 2.0 Hz, 3H), 4.09 (s, 3H), 2.16 (s, 6H), 2.09 (s, 6H). |
| 495 | 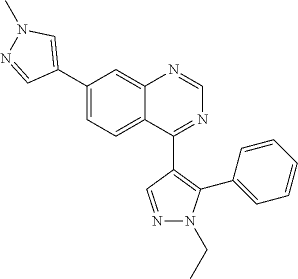 [SMILES: CCn1ncc(-c2ncnc3cc(-c4cnn(C)c4)ccc23)c1-c1ccccc1] | LC-MS: (ES, m/z): RT = 0.978 min, LCMS: m/z = 381 [M + 1]; ¹H NMR (300 MHz, DMSO-d₆) δ 9.04 (s, 1H), 8.44 (s, 1H), 8.16-8.07 (m, 2H), 8.04 (s, 1H), 7.92 (d, J = 8.8 Hz, 1H), 7.77 (dd, J = 8.8, 1.8 Hz, 1H), 7.44-7.29 (m, 5H), 4.17 (q, J = 7.2 Hz, 2H), 3.90 (s, 3H), 1.37 (t, J = 7.2 Hz, 3H). |
| 496 | 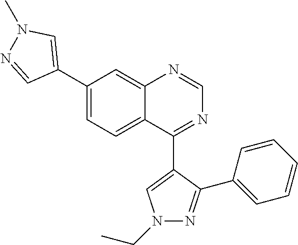 [SMILES: CCn1cc(-c2ncnc3cc(-c4cnn(C)c4)ccc23)c(-c2ccccc2)n1] | LC-MS: (ES, m/z): RT = 0.982 min, LCMS: m/z = 381 [M + 1]; ¹H NMR (300 MHz, DMSO-d₆) δ 9.16 (s, 1H), 8.45 (s, 1H), 8.33 (s, 1H), 8.17 (d, J = 1.7 Hz, 1H), 8.15 (s, 1H), 7.91-7.66 (m, 2H), 7.33 (dd, J = 6.6, 3.1 Hz, 2H), 7.23 (dt, J = 4.6, 2.8 Hz, 3H), 4.32 (q, J = 7.2 Hz, 2H), 3.90 (s, 3H), 1.53 (t, J = 7.3 Hz, 3H). |
| 497 | 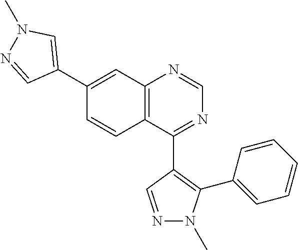 [SMILES: Cn1cc(-c2ccc3c(-c4cnn(C)c4-c4ccccc4)ncnc3c2)cn1] | LC-MS: (ES, m/z): RT = 1.301 min, LCMS: m/z = 367 [M + 1]; ¹H NMR (300 MHz, DMSO-d₆) δ 9.06 (s, 1H), 8.45 (s, 1H), 8.12 (dd, J = 7.4, 1.3 Hz, 2H), 8.00 (s, 1H), 7.89 (d, J = 8.8 Hz, 1H), 7.76 (dd, J = 8.7, 1.8 Hz, 1H), 7.44-7.27 (m, 5H), 3.90 (s, 6H). |
| 498 | 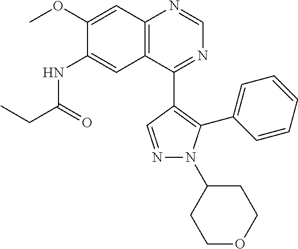 [SMILES: CCC(=O)Nc1cc2c(-c3cnn(C4CCOCC4)c3-c3ccccc3)ncnc2cc1OC] | LC-MS: (ES, m/z): RT = 1.087 min, LCMS: m/z = 458 [M + 1], ¹H NMR (300 MHz, DMSO-d₆) δ 9.37 (s, 1H), 8.87 (d, J = 4.8 Hz, 2H), 8.03 (s, 1H), 7.43-7.26 (m, 6H), 4.07- 3.90 (m, 6H), 3.37 (s, 2H), 2.46 (d, J = 7.5 Hz, 2H), 2.20 (td, J = 12.2, 4.4 Hz, 2H), 1.87 (d, J = 12.7 Hz, 2H), 1.11 (t, J = 7.5 Hz, 3H). |
| 499 | 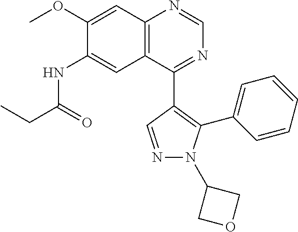 [SMILES: CCC(=O)Nc1cc2c(-c3cnn(C4COC4)c3-c3ccccc3)ncnc2cc1OC] | LC-MS: (ES, m/z): RT = 1.022 min, LCMS: m/z = 430 [M + 1], ¹H NMR (300 MHz, DMSO-d₆) δ 9.36 (s, 1H), 9.05 (s, 1H), 8.76 (s, 1H), 8.43 (s, 1H), 7.45 (s, 1H), 7.40- 7.29 (m, 2H), 7.24 (hept, J = 3.5 Hz, 3H), 5.77 (tt, J = 7.6, 6.2 Hz, 1H), 5.06 (p, J = 6.8 Hz, 4H), 4.07 (s, 3H), 2.43 (q, J = 7.5 Hz, 2H), 1.04 (t, J = 7.5 Hz, 3H). |
| 500 | 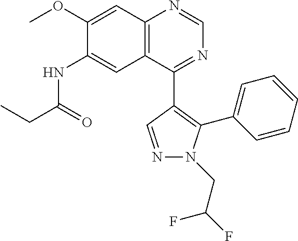 [SMILES: CCC(=O)Nc1cc2c(-c3cnn(CC(F)F)c3-c3ccccc3)ncnc2cc1OC] | LC-MS: (ES, m/z): RT = 1.154 min, LCMS: m/z = 438 [M + 1], ¹H NMR (300 MHz, DMSO-d₆) δ 9.37 (s, 1H), 8.92 (s, 1H), 8.84 (s, 1H), 8.08 (s, 1H), 7.41-7.25 (m, 6H), 6.46 (t, J = 3.9 Hz, 1H), 4.59 (td, J = 14.5, 3.9 Hz, 2H), 4.04 (s, 3H), 2.45 (d, J = 7.5 Hz, 2H), 1.10 (t, J = 7.5 Hz, 3H). |

### Experimental Data

### Biological Example 1. Biochemical EGFR Inhibition Assays

Inhibitory effects of the compounds of the disclosure were measured in biochemical assays that measure the phosphorylation activity of EGFR enzyme phosphorylates 2.5 micromolar 5-FAM-EEPLYWSFPAKKK-CONH₂ peptide substrate (ProfilerPro Kinase Peptide Substrate 22, PerkinElmer, Part #760366) in the presence of adenosine-5′-triphosphate (ATP) and varying concentrations of the test compound in 100 mM 2-[4-(2-hydroxyethyl) piperazin-1-yl]ethanesulfonic acid (HEPES), pH 7.5, 10 mM MgCl₂, 0.015% Brij-35, 1 mM dithiothreitol (DTT), 1.0% dimehylsulfoxide (DMSO). Assays were performed at 1.0 mM ATP or at ATP Km of the EGFR enzymes. Reactions proceeded until between 10% to 20% total peptides were phosphorylated at room temperature (25° C.) and were terminated with 35 mM 2,2′,2″,2″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following enzyme forms of EGFR were examples that were used in these assays:
EGFR WT (SignalChem, E10-112G)
EGFR L858R (SignalChem, E10-122BG)
EGFR (d746-750) (SignalChem, E10-122JG)
EGFR L858R C797S (SignalChem, E10-122ZG)
EGFR (d746-750) C797S (SignalChem, E10-122TG)

### Biological Example 2. Cellular EGFR Inhibition Assays

PC-9/A431 pEGFR AlphaLISA Assays

Inhibitory effects of compounds were evaluated in cellular assays that measure level of intracellular phosphorylation of EGFR in PC-9 (ECACC, #90071810, Milipore/Sigma) and A431 cell lines (ATCC, CRL-1555) using AlphaLISA sureFire ultra p-EGFR (Tyr1068) assay kit (PerkinElmer, ALSU-PEGFR-A50K). PC-9 cells were seeded at 3.125×10\hat{ }5 cells/ml in 40 μL phenol-free DMEM supplemented with 10% FBS per well of a 384 well plate (Corning, 3764), while A431 were seeded at 3.125×10\hat{ }5 cells/ml in 40 μL in phenol-free DMEM with 0.5% FBS. Cells were allowed to adhere overnight at 37° C./5% CO₂. On the next day, compounds were transferred at 4-fold, 10-point serial dilution from compound source plate to cell plates using liquid handler Echo550 and were incubated at 37° C./5% CO₂ for 4 hours. A431 cells were stimulated prior harvesting for 10 min with EGF at final concentration of 30 ng/ml in the incubator. Medium was removed from the plates and cells were lysed with 10 μL of 1× AlphaLISA lysis buffer (supplemented with 1× protease/phosphatase inhibitor cocktail) followed by shaking at 600 rpm for 30 minutes at room temperature. Lysates were transferred to Optiplate (Apricot designs) and 5 μl of 1× acceptor bead mix (prepared just before use) was added to each well followed by incubation at room temperature for 1.5-2 h in dark. Then 5 μL of freshly-made donor bead mix is added to each well under subdued lighting or green filters, was mixed well on the shaker and the plate was sealed and left for an overnight incubation at room temperature in dark. On the next day, the plate was read the Envision using standard AlphaLisa settings. Percent of pEGFR inhibition was plotted against log concentration of compounds to generate IC₅₀ values.

Biological assay data of the test compounds are provided in Table 2 below. For inhibitory activity against EGFR L858R C797S and EGFR (d746-750) C797S mutants, and for inhibition of phosphorylation of mutant EGFR in cells the following designations are used: ≤10 nM=A; 10.1-50 nM=B; and >50.1 nm=C.

TABLE 2

| | | Enz | Ext |
|---|---|---|---|
| | Enz | EGFR_Exon19- | pEGFR_PC- |
| Example | EGFR_L858R | del746-750 | 9 Ex19Del |
| number | IC₅₀ (nM) | IC₅₀ (nM) | IC₅₀ (nM) |
| 1 | 4.2 | 1.8 | 4.2 |
| 2 | 4.3 | 1.9 | 4.9 |
| 3 | 2.4 | 1.5 | 0.3 |
| 4 | 4.1 | 2.4 | 8.5 |
| 5 | 3.0 | 2.0 | 4.6 |
| 6 | 2.3 | 1.7 | 3.5 |
| 7 | 3.2 | 1.6 | 5.5 |
| 8 | 4.8 | 3.4 | 4.5 |
| 9 | 13.1 | 6.4 | 9.8 |
| 10 | 3.5 | 1.9 | 2.8 |
| 11 | 2.9 | 2.4 | 5.1 |
| 12 | 2.8 | 1.3 | 5.6 |
| 13 | 4.0 | 1.9 | 6.2 |
| 14 | 3.6 | 2.0 | 5.0 |
| 15 | 1.5 | 0.8 | 1.5 |
| 16 | 1.8 | 1.2 | 2.1 |
| 17 | 1.8 | 1.1 | 2.4 |
| 18 | 3.1 | 1.8 | 4.3 |
| 19 | 1.6 | 0.8 | 1.3 |
| 20 | 1.2 | 0.5 | 0.9 |
| 21 | 7.4 | 3.0 | 10.5 |
| 22 | 1.7 | 1.2 | 4.8 |
| 23 | 1.9 | 0.9 | 0.8 |
| 24 | 1.3 | 0.8 | 2.6 |
| 25 | 1.4 | 0.9 | 2.1 |
| 26 | 2.9 | 1.6 | 8.0 |
| 27 | 5.5 | 3.0 | 8.1 |
| 28 | 1.2 | 0.9 | 0.7 |
| 29 | 4.8 | 1.9 | 11.3 |
| 30 | 1.4 | 1.0 | 1.1 |
| 31 | 1.0 | 0.8 | 1.1 |
| 32 | 1.1 | 0.8 | 1.2 |
| 33 | 1.1 | 0.7 | 0.8 |
| 34 | 6.1 | 1.6 | 2.5 |
| 35 | 2.3 | 1.9 | 2.7 |
| 36 | 9.7 | 3.2 | 14 |
| 37 | 5.3 | 2.0 | 4.6 |
| 38 | 5.9 | 6.7 | 25.6 |
| 39 | 3.5 | 2.1 | 5.3 |
| 40 | 4.2 | 2.9 | 1.5 |
| 41 | 8.3 | 4.1 | 9.2 |
| 42 | 9.6 | 2.9 | 43.9 |
| 43 | 1.4 | 0.8 | 1.9 |
| 44 | 3.9 | 2.4 | 14.6 |
| 45 | 9.4 | 3.6 | 14.3 |
| 46 | 4.4 | 1.9 | 11.7 |
| 47 | 2.6 | 1.4 | 2.1 |
| 48 | 1.8 | 1.4 | 1.1 |
| 49 | 1.3 | 0.6 | 1.2 |
| 50 | 0.5 | 0.3 | 0.3 |
| 51 | 6.9 | 5.3 | 30.6 |
| 52 | 12.3 | 2.7 | 6.3 |
| 53 | 12.8 | 1.1 | 2.7 |
| 54 | 10.6 | 1.0 | 1.6 |
| 55 | 5.0 | 2.0 | 3.6 |
| 56 | 3.4 | 2.1 | 3.0 |
| 57 | 2.3 | 1.7 | 2.2 |
| 58 | 4.5 | 2.5 | 5.1 |
| 59 | 3.3 | 2.0 | 4.2 |
| 60 | 5.1 | 3.6 | 3.6 |
| 61 | 4.5 | 2.3 | 7.9 |
| 62 | 4.2 | 2.5 | 8.7 |
| 63 | 4.8 | 2.4 | 2.5 |
| 64 | 6.1 | 2.1 | 13.7 |
| 65 | 6.1 | 2.5 | 2.5 |
| 66 | 2.9 | 2.2 | 5.2 |
| 67 | 8.3 | 2.8 | 12.4 |
| 68 | 6.3 | 2.7 | 3.0 |
| 69 | 7.1 | 3.1 | 5.8 |
| 70 | 2.7 | 1.7 | 1.6 |
| 71 | 4.5 | 2.5 | 6.8 |
| 72 | 3.8 | 1.7 | 5.1 |
| 73 | 2.9 | 1.6 | 11.1 |
| 74 | 829.1 | 661.9 | 4.5 |
| 75 | 3.6 | 2.2 | 3.6 |
| 76 | 2.1 | 0.9 | 1.4 |
| 77 | 7.2 | 3.5 | 9.7 |
| 78 | 7.0 | 2.4 | 8.1 |
| 79 | 6.5 | 3.1 | 9.60 |
| 80 | 6.0 | 3.1 | 9.6 |
| 81 | 6.1 | 2.2 | 5.2 |
| 82 | 5.3 | 3.3 | 3.9 |
| 83 | 3.0 | 2.2 | 4.9 |
| 84 | 4.9 | 3.3 | 6.5 |
| 85 | 3.9 | 2.7 | 13.3 |
| 86 | 5.0 | 2.5 | 5.8 |
| 87 | 4.9 | 2.1 | 5.7 |
| 88 | 3.2 | 1.9 | 7.5 |
| 89 | 5.4 | 1.9 | 5.7 |
| 90 | 14.0 | 8.5 | 31.6 |
| 91 | 9.3 | 5.6 | 9.5 |
| 92 | 9.3 | 3.4 | 13.4 |
| 93 | 4.3 | 2.5 | 4.3 |
| 94 | 6.4 | 3.2 | 11.3 |
| 95 | 3.0 | 1.7 | 2.4 |
| 96 | 1.6 | 1.1 | 1.9 |
| 97 | 7.8 | 4.9 | 12.4 |
| 98 | 6.8 | 3.4 | 9.3 |
| 99 | 2.0 | 1.1 | 4.2 |
| 100 | 2.9 | 2.0 | 7.6 |
| 101 | 3.2 | 1.9 | 5.7 |
| 102 | 2.0 | 1.2 | 4.1 |
| 103 | 1.7 | 0.6 | 2.7 |
| 104 | 2.1 | 1.3 | 5.2 |
| 105 | 3.3 | 1.3 | 6.5 |
| 106 | 3.4 | 1.8 | 10.9 |
| 107 | 1.3 | 0.6 | 3.3 |
| 108 | 1.3 | 0.9 | 2.7 |
| 109 | 3.8 | 2.2 | 4.7 |
| 110 | 1.8 | 1.4 | 2.5 |
| 111 | 1.4 | 0.7 | 2.5 |
| 112 | 1.9 | 1.2 | 3.8 |
| 113 | 5.5 | 2.4 | 11.2 |
| 114 | 2.9 | 2.3 | 28.9 |
| 115 | 2.3 | 1.2 | 10.6 |
| 116 | 1.2 | 1.1 | 11.5 |
| 117 | 1.9 | 0.6 | 3.4 |
| 118 | 3.9 | 2.4 | 6.0 |
| 119 | 3.9 | 2.1 | 13.6 |
| 120 | 2.9 | 0.8 | 7.3 |
| 121 | 7.6 | 2.7 | 13.8 |
| 122 | 1.9 | 1.3 | 2.8 |
| 123 | 1.6 | 1.0 | 3.0 |
| 124 | 1.3 | 0.9 | 3.0 |
| 125 | 1.4 | 0.9 | 2.1 |
| 126 | 1.9 | 0.9 | 3.2 |
| 127 | 2.6 | 1.4 | 2.6 |
| 128 | 3.6 | 2.5 | 12.5 |
| 129 | 4.3 | 1.1 | 2.7 |
| 130 | 21.5 | 5.2 | 16.1 |
| 131 | 1.6 | 1.9 | 3.3 |
| 132 | 1.4 | 0.9 | 0.6 |
| 133 | 1.1 | 0.5 | 2.4 |
| 134 | 4.0 | 2.1 | 11.6 |
| 135 | 1.2 | 0.7 | 3.1 |
| 136 | 0.8 | 0.4 | 1.3 |
| 137 | 3.9 | 2.2 | 3.6 |
| 138 | 5.6 | 2.1 | 4.6 |
| 139 | | | 5.1 |
| 140 | 4.5 | 1.9 | 6.7 |
| 141 | 7.1 | 1.9 | 7.6 |
| 142 | 3.2 | 2.8 | 7.1 |
| 143 | 4.5 | 1.7 | 3.2 |
| 144 | | | 8.3 |
| 145 | | | 5.9 |
| 146 | 8.2 | 4.0 | 10.8 |
| 147 | 13.7 | 1.2 | 2.3 |
| 148 | 7.0 | 3.0 | 8.3 |
| 149 | 4.9 | 2.1 | 12.8 |
| 150 | 2.2 | 1.2 | 1.5 |
| 151 | 8.2 | 1.9 | 8.7 |
| 152 | 2.0 | 1.0 | 1.9 |
| 153 | 1.0 | 0.5 | 0.9 |
| 154 | 3.8 | 1.1 | 13 |
| 155 | 7.3 | 2.0 | 5.1 |
| 156 | 3.2 | | 2.4 |
| 157 | 1.0 | 0.6 | 1.0 |
| 158 | 1.7 | 1.4 | 0.2 |
| 159 | 3.0 | 1.5 | 3.8 |
| 160 | 20.0 | 2.9 | 8.3 |
| 161 | 4.6 | 2.5 | 8.1 |
| 162 | 7.0 | 2.6 | 6.0 |
| 163 | 3.6 | 1.4 | 5.5 |
| 164 | 2.9 | 1.7 | 6.4 |
| 165 | 3.4 | 1.7 | 10.2 |
| 166 | 1.7 | 0.7 | 3.2 |
| 167 | 1.9 | 1.1 | 1.7 |
| 168 | 1.5 | 1.0 | 0.8 |
| 169 | 1.7 | 0.9 | 1.9 |
| 170 | 2.0 | 0.9 | 1.2 |
| 171 | 5.2 | 2.9 | 4.8 |
| 172 | 6.7 | 2.2 | 11.7 |
| 173 | 9.0 | 3.6 | 13.8 |
| 174 | 2.8 | 2.3 | 3.4 |
| 175 | 1.6 | 1.0 | 1.2 |
| 176 | 5.4 | 3.0 | 11 |
| 177 | 4.3 | 2.1 | 6.1 |
| 178 | 6.2 | 3.3 | 5.4 |
| 179 | 5.5 | 2.8 | 4.0 |
| 180 | 3.8 | 2.2 | 4.0 |
| 181 | 4.3 | 2.7 | 4.5 |
| 182 | 4.1 | 1.8 | 5.2 |
| 183 | 8.0 | 3.6 | 10.7 |
| 184 | 3.5 | 1.8 | 7.9 |
| 185 | 2.1 | 1.3 | 3.1 |
| 186 | 3.2 | 2.3 | 4.0 |
| 187 | 2.6 | 1.5 | 4.0 |
| 188 | 2.0 | 0.8 | 1.3 |
| 189 | 4.0 | 1.9 | 4.3 |
| 190 | 1.1 | 0.8 | 1.5 |
| 191 | 3.8 | 1.8 | 6.4 |
| 192 | 1.1 | 0.7 | 1.6 |
| 193 | 1.5 | 0.7 | 2.9 |
| 194 | 1.4 | 0.7 | 1.7 |
| 195 | 3.8 | 1.6 | 5.9 |
| 196 | 1.7 | 0.9 | 1.5 |
| 197 | 1.8 | 0.8 | 2.5 |
| 198 | 1.3 | 0.8 | 1.1 |
| 199 | 1.6 | 1.1 | 1.9 |
| 200 | 5.7 | 2.4 | 8.4 |
| 201 | 2.0 | 1.4 | 3.8 |
| 202 | 0.9 | 0.6 | 0.6 |
| 203 | 2.1 | 1.1 | 4.9 |
| 204 | 2.1 | 1.3 | 1.4 |
| 205 | 1.2 | 0.8 | 0.8 |
| 206 | 0.8 | 0.4 | 0.5 |
| 207 | 1.4 | 0.8 | 1.0 |
| 208 | 1.1 | 0.7 | 1.8 |
| 209 | 1.5 | 0.8 | 0.9 |
| 210 | 1.1 | 0.6 | 1.7 |
| 211 | 2.8 | 1.5 | 3.3 |
| 212 | 0.4 | 0.2 | 1.0 |
| 213 | 0.9 | 0.6 | 0.6 |
| 214 | 1.6 | 0.9 | 1.2 |
| 215 | 2.7 | 1.6 | 1.1 |
| 216 | 2.1 | 0.9 | 3.8 |
| 217 | 1.7 | 0.9 | 1.2 |
| 218 | 2.1 | 1.0 | 2.7 |
| 219 | 1.9 | 1.1 | 1.7 |
| 220 | 5.0 | 1.7 | 4.7 |
| 221 | 1.8 | 1.0 | 2.3 |
| 222 | 1.5 | 0.9 | 1.5 |
| 223 | 2.9 | 2.1 | 3.2 |
| 224 | 5.1 | 3.0 | 3.9 |
| 225 | 14.0 | 9.0 | 5.1 |
| 226 | 1.6 | 1.0 | 1.4 |
| 227 | 15.2 | 2.7 | 6.7 |
| 228 | 2.8 | 1.7 | 5.6 |
| 229 | 3.5 | 1.7 | 3.7 |
| 230 | 1.5 | 0.9 | 2.3 |
| 231 | 1.5 | 1.0 | 2.4 |
| 232 | 2.7 | 1.5 | 4.1 |
| 233 | 2.9 | 1.7 | 2.5 |
| 234 | 1.9 | 1.1 | 2.8 |
| 235 | 1.6 | 0.9 | 1.5 |
| 236 | 3.8 | 1.4 | 3.9 |
| 237 | 5.4 | 2.0 | 6.6 |
| 238 | 12.0 | 8.2 | 26 |
| 239 | 4.1 | 1.7 | 4.0 |
| 240 | 1.8 | 0.8 | 1.4 |
| 241 | 3.0 | 1.5 | 2 |
| 242 | 7.0 | 2.1 | 6.6 |
| 243 | 4.9 | 2.4 | 4.4 |
| 244 | 2.2 | 1.3 | 2.3 |
| 245 | 1.9 | 1.0 | 2.6 |
| 246 | 5.6 | 2.5 | 7.8 |
| 247 | 1.0 | 0.7 | 0.8 |
| 248 | 2.0 | 1.5 | 1.3 |
| 249 | 2.2 | 1.4 | 2.1 |
| 250 | 1.4 | 1.0 | 0.9 |
| 251 | | | 3.5 |
| 252 | | | 3.2 |
| 253 | 2.4 | 1.9 | 3.9 |
| 254 | 2.1 | 0.8 | 2.5 |
| 255 | 2.7 | 1.8 | 3.8 |
| 256 | 0.8 | 0.4 | 2.0 |
| 257 | 1.5 | 0.9 | 4.8 |
| 258 | 3.8 | 0.9 | 4.2 |
| 259 | 2.6 | 1.1 | 4.2 |
| 260 | 6.8 | 1.2 | 3.2 |
| 261 | 2.8 | 1.3 | 6.1 |
| 262 | 1.1 | 0.6 | 1.1 |
| 263 | 1.7 | 0.7 | 2.7 |
| 264 | 0.6 | 0.4 | 0.3 |
| 265 | 3.2 | 1.8 | 2.3 |
| 266 | 1.5 | 1.0 | 4.9 |
| 267 | 108.7 | 9.8 | 12.1 |
| 268 | 1.8 | 1.1 | 0.8 |
| 269 | 1.3 | 0.9 | 0.9 |
| 270 | 2.1 | 1.4 | 1.4 |
| 271 | 1.3 | 0.8 | 1.0 |
| 272 | 1.2 | 1.0 | 0.5 |
| 273 | 1.5 | 1.0 | 0.6 |
| 274 | 1.5 | 0.9 | 0.6 |
| 275 | 1.9 | 1.3 | 1.9 |
| 276 | 2.0 | 1.6 | 1.3 |
| 277 | 3.7 | 1.9 | 6.0 |
| 278 | 4.5 | 2.3 | 7.8 |
| 279 | 3.3 | 1.8 | 6.5 |
| 280 | 2.4 | 1.7 | 2.5 |
| 281 | 2.2 | 1.4 | 2.3 |
| 282 | 2.2 | 1.6 | 0.7 |
| 283 | 1.7 | 0.9 | 1.1 |
| 284 | 8.7 | 4.7 | 18.7 |
| 285 | 1.3 | 0.8 | 0.5 |
| 286 | 3.5 | 1.6 | 4.3 |
| 287 | 4.5 | 1.2 | 13 |
| 288 | 1.9 | 1.2 | 2.0 |
| 289 | 1.5 | 0.8 | 1.5 |
| 290 | 1.3 | 0.9 | 1.5 |
| 291 | 1.6 | 1.0 | 1.7 |
| 292 | 145.0 | 3.2 | 9.6 |
| 293 | 277.0 | 3.4 | 8.9 |
| 294 | 1.0 | 0.5 | 1.1 |
| 295 | 4.6 | 2.1 | 5.3 |
| 296 | 1.4 | 0.7 | 1.1 |
| 297 | 0.9 | 0.5 | 0.9 |
| 298 | 1.6 | 0.9 | 1.0 |
| 299 | 15.3 | 5.4 | 7.1 |
| 300 | 5.0 | 1.7 | 3.5 |
| 301 | 2.7 | 2.0 | 0.8 |
| 302 | 1.4 | 0.7 | 1.6 |
| 303 | 11.0 | 2.9 | 8.8 |
| 304 | 1.8 | 1.0 | 1.2 |
| 305 | 5.1 | 3.3 | 3.5 |
| 306 | 1.4 | 1.1 | 0.6 |
| 307 | 1.7 | 0.9 | 1.4 |
| 308 | 7.3 | 3.5 | 6.1 |
| 309 | 3.1 | 1.9 | 2.8 |
| 310 | 3.4 | 1.3 | 3.1 |
| 311 | 3.4 | 1.9 | 2.1 |
| 312 | 6.9 | 3.2 | 8.8 |
| 313 | 2.7 | 0.8 | 3.8 |
| 314 | 1.4 | 0.8 | 1.1 |
| 315 | 1.7 | 1.1 | 0.6 |
| 316 | 3.5 | 0.8 | 3.1 |
| 317 | 2.7 | 1.2 | 1.5 |
| 318 | 0.7 | 0.4 | 0.7 |
| 319 | 1.1 | 0.7 | 0.4 |
| 320 | 2.0 | 0.6 | 1.1 |
| 321 | 5.2 | 2.7 | 5.3 |
| 322 | 2.1 | 1.7 | 1.6 |
| 323 | 3.3 | 1.6 | 3.9 |
| 324 | 3.8 | 2.0 | 2.6 |
| 325 | 1.7 | 1.0 | 2.0 |
| 326 | 1.2 | 0.8 | 1.0 |
| 327 | 10.4 | 5.1 | 10.4 |
| 328 | 2.3 | 1.1 | 4.5 |
| 329 | 1.4 | 0.9 | 1.3 |
| 330 | 1.0 | 0.7 | 0.8 |
| 331 | 1.7 | 1.0 | 1.5 |
| 332 | 2.3 | 1.6 | 2.1 |
| 333 | 1.3 | 0.8 | 1.7 |
| 334 | 2.4 | 1.5 | 2.2 |
| 335 | 2.9 | 1.6 | 3.0 |
| 336 | | | 4.0 |
| 337 | | | 5.9 |
| 338 | 1.9 | 1.2 | 2.3 |
| 339 | 1.7 | 1.0 | 0.8 |
| 340 | 3.3 | 1.6 | 2.5 |
| 341 | 6.4 | 3.0 | 8.3 |
| 342 | 6.7 | 3.5 | 11.2 |
| 343 | | | 4.5 |
| 344 | 1.6 | 1.0 | 0.8 |
| 345 | 4.7 | 1.9 | 7.8 |
| 346 | 2.1 | 1.3 | 0.9 |
| 347 | 2.4 | 1.2 | 0.6 |
| 348 | 3.2 | 1.7 | 3.4 |
| 349 | 2.0 | 1.0 | 3.5 |
| 350 | 3.1 | 1.5 | 4.4 |
| 351 | 3.1 | 1.7 | 4.6 |
| 352 | 3.6 | 1.7 | 4.9 |
| 353 | 2.3 | 1.3 | 1.6 |
| 354 | 2.6 | 1.2 | 0.8 |
| 355 | 1.7 | 1.0 | 0.8 |
| 356 | 1.9 | 1.1 | 1.2 |
| 357 | 2.2 | 1.1 | 4.4 |
| 358 | 1.8 | 1.1 | 1.0 |
| 359 | 1.6 | 0.7 | 1.6 |
| 360 | 1.8 | 1.0 | 2.7 |
| 361 | 1.6 | 0.9 | 1.2 |
| 362 | 1.1 | 0.5 | 0.7 |
| 363 | 5.4 | 1.7 | 3.5 |
| 364 | 2.1 | 1.0 | 4.0 |
| 365 | 1.8 | 0.9 | 2.0 |
| 366 | 1.4 | 0.8 | 1.4 |
| 367 | 1.6 | 0.9 | 0.5 |
| 368 | 3.9 | 1.5 | 4.6 |
| 369 | 1.3 | 0.7 | 0.9 |
| 370 | 1.1 | 0.7 | 0.4 |
| 371 | 1.4 | 0.9 | 0.8 |
| 372 | 1.6 | 0.7 | 1.5 |
| 373 | 2.2 | 0.8 | 2.3 |
| 374 | 4.3 | 1.4 | 3.6 |
| 375 | 2.4 | 0.9 | 2.5 |
| 376 | 1.3 | 0.4 | 2.7 |
| 377 | 2.2 | 1.3 | 2.8 |
| 378 | 1.3 | 0.7 | 1.2 |
| 379 | 2.5 | 0.7 | 0.8 |
| 380 | 2.5 | 0.7 | 1.4 |
| 381 | 1.9 | 0.7 | 1.4 |
| 382 | 2.4 | 0.6 | 1.6 |
| 383 | 4.5 | 1.7 | 5.7 |
| 384 | 1.1 | 0.3 | 1.1 |
| 385 | 1.7 | 0.4 | 0.7 |
| 386 | 2.1 | 1.1 | 1.7 |
| 387 | 2.0 | 0.9 | 1.5 |
| 388 | 4.8 | 0.5 | 0.4 |
| 389 | 6.4 | 0.7 | 0.4 |
| 390 | 9.3 | 3.9 | 8.7 |
| 391 | 2.5 | 1.3 | 0.7 |
| 392 | 6.1 | 2.2 | 5.9 |
| 393 | 2.1 | 0.9 | 3.5 |
| 394 | 6.9 | 2.7 | 9.3 |
| 395 | 3.1 | 1.2 | 5.3 |
| 396 | 1.7 | 1.1 | 0.5 |
| 397 | 2.3 | 1.0 | 1.6 |
| 398 | 2.6 | 1.4 | 1.3 |
| 399 | 1.2 | 0.9 | 1.3 |
| 400 | 141.0 | 1.6 | 2.0 |
| 401 | 53.0 | 1.2 | 2.3 |
| 402 | 3.1 | 1.3 | 3.8 |
| 403 | 4.2 | 1.2 | 5 |
| 404 | 5.9 | 0.7 | 3.6 |
| 405 | 2.0 | 0.8 | 1.7 |
| 406 | 1.2 | 0.7 | 0.9 |
| 407 | 2.1 | 0.7 | 1.4 |
| 408 | 1.5 | 0.7 | 0.7 |
| 409 | 1.0 | 0.6 | 0.8 |
| 410 | 2.9 | 1.1 | 2.5 |
| 411 | 2.6 | 1.3 | 2.3 |
| 412 | 26.0 | | 6.7 |
| 413 | 10.0 | | 23.2 |
| 414 | 2.1 | 1.3 | 1.2 |
| 415 | 3.0 | 1.5 | 7.8 |
| 416 | 1.8 | 1.0 | 0.8 |
| 417 | 1.5 | 0.8 | 0.9 |
| 418 | 1.8 | 0.6 | 1.8 |
| 419 | 1.6 | 1.0 | 0.4 |
| 420 | 30.4 | 1.7 | 5.9 |
| 421 | 1.3 | 1.8 | 1.9 |
| 422 | 6.7 | 1.0 | 4.1 |
| 423 | 4.1 | 1.6 | 3.8 |
| 424 | 2.3 | 1.2 | 1.5 |
| 425 | 2.8 | 1.0 | 0.8 |
| 426 | 2.8 | 1.1 | 1.3 |
| 427 | 2.0 | 0.7 | 2.1 |
| 428 | 3.9 | 1.2 | 7.7 |
| 429 | 5.9 | 2.7 | 11.4 |
| 430 | 19.0 | 3.7 | 16 |
| 431 | 1.4 | 0.6 | 1.2 |
| 432 | 1.9 | 0.9 | 2.5 |
| 433 | 24.9 | 1.6 | 10.5 |
| 434 | 6.8 | 2.0 | 3.5 |
| 435 | 25.8 | 3.1 | 9.7 |
| 436 | 8.0 | 1.0 | 4.6 |
| 437 | 1.4 | 0.8 | 4.2 |
| 438 | 1.2 | 0.8 | 0.3 |
| 439 | 0.7 | 0.4 | 1.7 |
| 440 | 0.9 | | 1.1 |
| 441 | 1.1 | 0.7 | 0.40 |
| 442 | 1.4 | 1.1 | 0.4 |
| 443 | 0.8 | 0.4 | 0.3 |
| 444 | 0.5 | 0.4 | 0.5 |
| 445 | 1.5 | 1.0 | 2.7 |
| 446 | 0.9 | 0.6 | 0.7 |
| 447 | 1.6 | 0.9 | 1.6 |
| 448 | | | 6.5 |
| 449 | 1.3 | 0.9 | 1.9 |
| 450 | 1.2 | 0.7 | 1.0 |
| 451 | 3.5 | 2.0 | 2.8 |
| 452 | 28.9 | 5.3 | 14.8 |
| 453 | 1.3 | 0.7 | 0.4 |
| 454 | 18.1 | 4.8 | 11 |
| 458 | | | |
| 459 | 3.8 | 1.0 | 1.2 |
| 460 | 5.7 | 1.5 | 3.2 |
| 461 | 12.7 | 2.9 | 7.2 |
| 463 | 3.9 | 0.8 | 1.7 |
| 464 | 0.9 | 0.3 | 1.4 |
| 465 | 4.9 | 0.8 | 3.5 |
| 466 | 39.0 | 7.3 | 31.2 |
| 467 | 5.5 | 1.3 | 5.3 |
| 468 | 60.4 | 7.4 | 22.0 |
| 469 | 11.1 | 2.4 | 2.7 |
| 470 | 26.0 | 4.5 | 23.8 |
| 472 | 7.6 | 1.9 | 2.7 |
| 474 | 5.6 | 2.0 | 3.6 |
| 475 | 65.5 | 21.0 | 71.6 |
| 476 | 5.4 | 1.3 | |
| 477 | 7.7 | 9.0 | 25.0 |
| 478 | 10.5 | 2.8 | 3.6 |
| 479 | 2.7 | 2.1 | 25.9 |
| 480 | 5.6 | 4.4 | 69.1 |
| 481 | 70.5 | 78 | 953 |
| 482 | 207.0 | 97.0 | 320.0 |
| 483 | 748.5 | 299.5 | 1706 |
| 484 | 48.7 | 13.4 | 184 |
| 485 | 121 | 76.3 | 1208 |
| 486 | 2.2 | 0.7 | 1.5 |
| 487 | 50.0 | 19.5 | 173.1 |
| 488 | 416.0 | 126 | 980.8 |
| 489 | 6.9 | 3.9 | 13.8 |
| 490 | 82 | 237 | 836 |
| 492 | 70.7 | 47.3 | 636 |
| 493 | 92.4 | 31.9 | 257.6 |
| 494 | 4.4 | 1.2 | 3.6 |
| 495 | 1.2 | 2.3 | 15.3 |
| 496 | 0.7 | 0.6 | 0.4 |
| 497 | 1.4 | 1.8 | 38.6 |
| 498 | 306 | 349 | 589 |
| 499 | 17 | 33 | 495 |
| 500 | 2.9 | 7.0 | 55.7 |
| Preparation 190 | 1.3 | 0.8 | 5.4 |

Additional Examples 455-457 and 458A were also prepared and tested, and were found to have inhibitory activities of greater than 10 μM in all three of the EGFR L858R. EGFR (d746-750) and EGFR PC9 biological assays described above in Biological Examples 1 and 2, see Table 3 below.

TABLE 3

| Example | |
|---|---|
| Number | Structure |
| 455 | 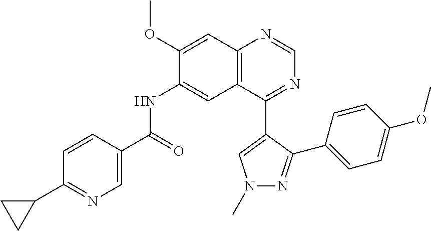 [SMILES: COc1ccc(-c2nn(C)cc2-c2ncnc3cc(OC)c(NC(=O)c4ccc(C5CC5)nc4)cc23)cc1] |
| 456 | 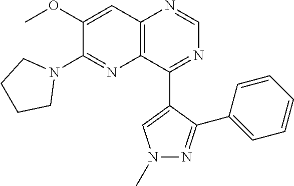 [SMILES: COc1cc2ncnc(-c3cn(C)nc3-c3ccccc3)c2nc1N1CCCC1] |
| 457 | 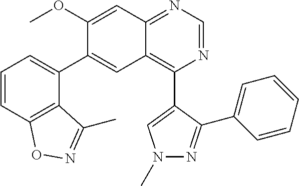 [SMILES: COc1cc2ncnc(-c3cn(C)nc3-c3ccccc3)c2cc1-c1cccc2onc(C)c12] |
| 458A | 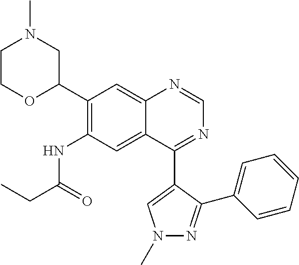 [SMILES: CCC(=O)Nc1cc2c(-c3cn(C)nc3-c3ccccc3)ncnc2cc1C1CN(C)CCO1] |

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

What is claimed is:

1. A compound of Formula (A): 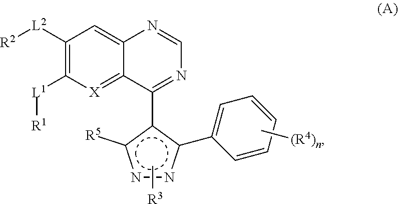
or a pharmaceutically acceptable salt thereof, wherein
X is CR^{x} or N;
R^{x} is H or F;
L¹ is a bond, NH, —NHC(O)—*, —NHC(O)O—*, O, or —OC(O)—*; wherein -* represents the point which attaches to R¹;
L² is a bond or O;
R¹ is H; or
C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl; or
C₃-C₈cycloalkyl, phenyl, 4 to 12 membered heterocyclyl, or 5 to 12 membered heteroaryl, wherein the cycloalkyl, phenyl, heterocyclyl and heteroaryl represented by R¹ are each optionally substituted with 1 to 4 groups independently selected from R¹¹;
each R¹¹ is independently selected from halo, deuterium, OR^{a}, C(O)R^{a}, C(O)NR^{a}R^{b}, NR^{a}C(O)OR^{a}, NR^{a}R^{b}, S(O)₂R^{a}, C₁-C₄alkyl, C₃-C₆cycloalkyl, phenyl, 4 to 12 membered heterocyclyl and 5 to 12 membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄alkyl, OR^{a} and NR^{a}R^{b}, or two R¹¹ which are attached to the same carbon atom are taken together to form ═O;
R² is
H, C₁-C₄alkyl, C₃-C₈cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl; or 4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl;
R³ is attached to either nitrogen atom in the pyrazole ring, and is selected from H, deuterium, C₁-C₄alkyl, C₃-C₆cycloalkyl, and 4 to 6 membered heterocyclyl, wherein the alkyl, cycloalkyl and heterocyclyl represented by R³ are each optionally substituted with 1 to 4 groups independently selected from halo, deuterium, OR^{a}, NR^{a}R^{b} and C₃-C₆cycloalkyl:
each R⁴ is independently selected from halo, deuterium and OR^{a};
R⁵ is selected from H, deuterium, halo and C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl
each R^{a} is independently selected from H, deuterium, C₁-C₄alkyl, C₁-C₄haloalkyl, C₃-C₆cycloalkyl and 4 to 6 membered heterocyclyl;
each R^{b} is independently selected from H, deuterium and C₁-C₄alkyl; and
n is 0, 1, 2, 3, 4 or 5.

2. The compound of claim 1, wherein the compound is of Formula (B) or (C): 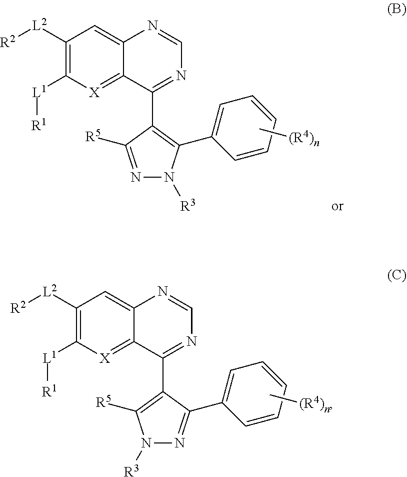
or a pharmaceutically acceptable salt thereof.

3. The compound of any one of claims 1-2, or a pharmaceutically acceptable salt thereof, wherein when L¹ and L² are each a bond, R¹ is H, R² is not H.

4. The compound of any one of claims 1-3, or a pharmaceutically acceptable salt thereof, wherein
R² is C₁-C₄alkyl, C₃-C₈cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl: or 4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl.

5. The compound of any one of claims 1-4, or a pharmaceutically acceptable salt thereof, wherein R⁵ is H, F or methyl.

6. A compound of Formula (I): 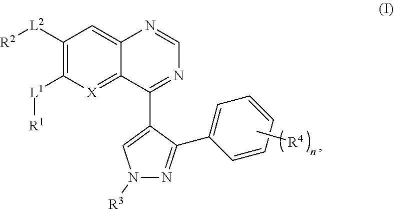
or a pharmaceutically acceptable salt thereof, wherein:
X is CR^{x} or N;
R^{x} is H or F;
L¹ is a bond, NH, —NHC(O)—*, —NHC(O)O—*, O, or —OC(O)—*; wherein -* represents the point which attaches to R¹;
L² is a bond or O;
R¹ is H; or
C₁-C₄alkyl optionally substituted with 1 to 4 groups independently selected from halo, deuterium, OR^{a}, NR^{a}R^{b}, C₃-C₆cycloalkyl, 4 to 12 membered heterocyclyl and 5 to 10 membered heteroaryl, wherein the heterocyclyl and heteroaryl are each optionally substituted with 1 to 4 groups independently selected from halo, deuterium and C₁-C₄alkyl; or
C₃-C₈cycloalkyl, phenyl, 4 to 12 membered heterocyclyl, or 5 to 12 membered heteroaryl, wherein the cycloalkyl, phenyl, heterocyclyl and heteroaryl represented by R¹ are each optionally substituted with 1 to 4 groups independently selected from R¹¹;
each R¹¹ is independently selected from halo, deuterium, OR^{a}, C(O)R^{a}, C(O)NR^{a}R^{b}, NR^{a}C(O)OR^{a}, NR^{a}R^{b}, S(O)₂R^{a}, C₁-C₄alkyl, C₃-C₆cycloalkyl, phenyl, 4 to 12 membered heterocyclyl and 5 to 12 membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄alkyl, OR^{a} and NR^{a}R^{b}, or two R¹¹ which are attached to the same carbon atom are taken together to form ═O;
R² is:
C₁-C₄alkyl, C₃-C₆cycloalkyl, C₂-C₄alkenyl or C₂-C₄alkynyl, each of which is optionally substituted with 1 to 4 groups independently selected from halo, deuterium, OR^{a}, NR^{a}R^{b} and 4 to 12 membered heterocyclyl optionally substituted with 1 to 4 groups independently selected from halo, deuterium and C₁-C₄alkyl; or 4 to 12 membered heterocyclyl or 5 to 12 membered heteroaryl, wherein the heterocyclyl and heteroaryl represented by R² are each optionally substituted with 1 to 4 groups selected from deuterium, C₁-C₄alkyl, C(O)R^{a} and 4 to 12 membered heterocyclyl which is optionally substituted with 1 to 4 groups selected from halo, deuterium and C₁-C₄alkyl;
R³ is H, deuterium, C₁-C₄alkyl, C₃-C₆cycloalkyl or 4 to 6 membered heterocyclyl, wherein the alkyl, cycloalkyl and heterocyclyl represented by R³ are each optionally substituted with 1 to 4 groups independently selected from halo, deuterium, OR^{a}, NR^{a}R^{b} and C₃-C₆cycloalkyl;
each R⁴ is independently selected from halo, deuterium and OR^{a};
each R^{a} is independently selected from H, deuterium, C₁-C₄alkyl, C1-C₄haloalkyl, C₃-C₆cycloalkyl and 4 to 6 membered heterocyclyl;
each R^{b} is independently selected from H, deuterium and C₁-C₄alkyl; and
n is 0, 1, 2, 3, 4 or 5.

7. The compound of claim 6, wherein the compound is represented by one of the following structural formulas: 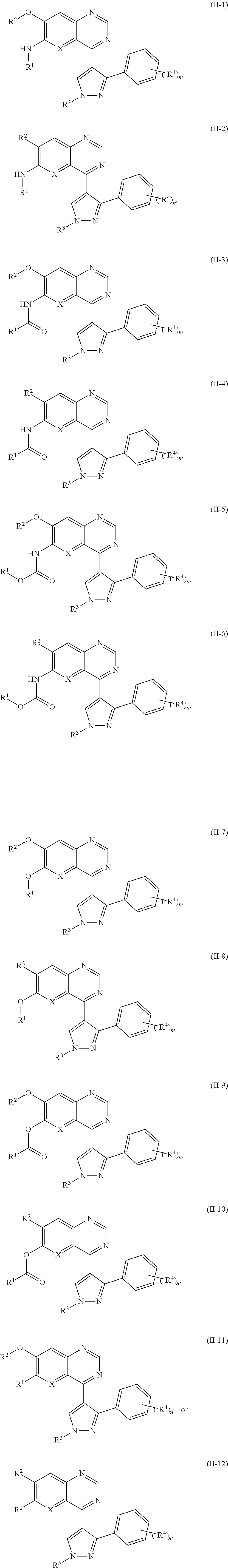
or a pharmaceutically acceptable salt thereof.

8. The compound of claim 6, wherein the compound is represented by one of the following structural formulas: 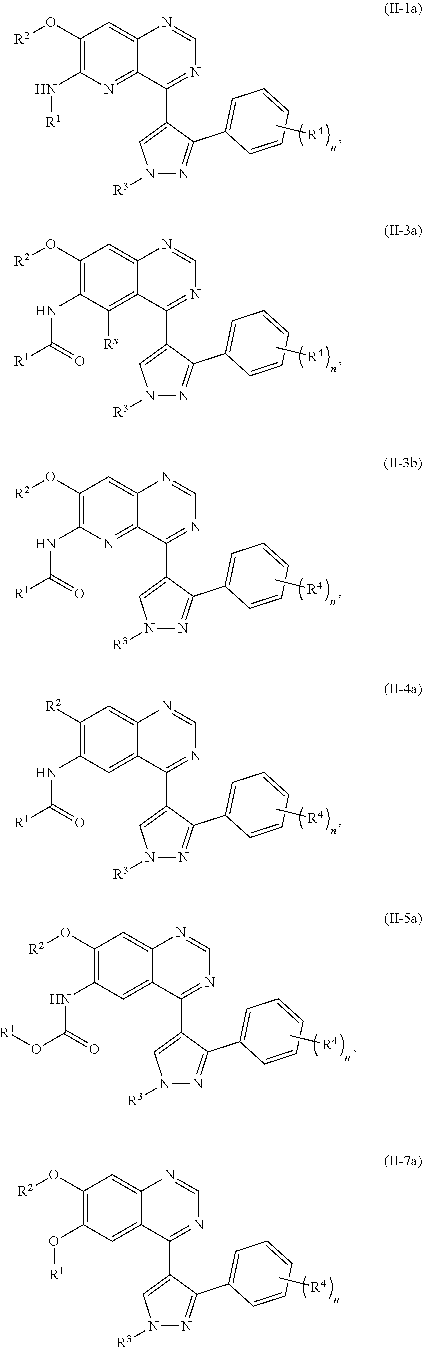 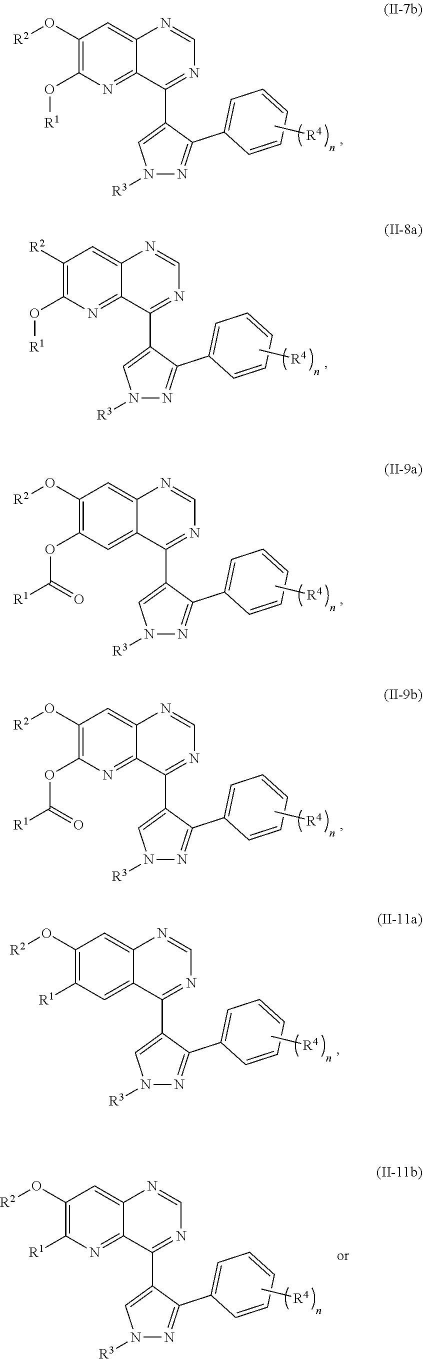 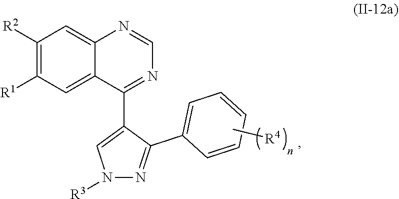
or a pharmaceutically acceptable salt thereof.

9. The compound of any one of claims 1-8, or a pharmaceutically acceptable salt thereof, wherein R^{x} is H.

10. The compound of any one of claims 1-9, or a pharmaceutically acceptable salt thereof, wherein:
R¹ is H: or
C₁-C₃alkyl optionally substituted with 1 to 2 groups independently selected from halo, OR^{a}, NR^{a}R^{b}, C₃-C₈cycloalkyl, 4 to 6 membered heterocyclyl and 5 to 6 membered heteroaryl, wherein the heterocyclyl and heteroaryl are each optionally substituted with 1 to 2 groups independently selected from halo and —CH₃; or
C₃-C₈cycloalkyl, phenyl, 4 to 9 membered heterocyclyl or 5 to 10 membered heteroaryl, wherein the cycloalkyl, phenyl, heterocyclyl and heteroaryl in the group represented by R¹ are each optionally substituted with 1 to 3 groups independently selected from R¹¹; and
each R^{a} is independently H or —CH₃; and each R^{b} is —CH₃.

11. The compound of any one of claims 1-10, or a pharmaceutically acceptable salt thereof, wherein:
R¹ is H; or
R¹ is selected from —CH₃, —CH₂CH₃, —CH₂CH₃CH₃ and —CH(CH₃)₂, each of which is optionally substituted with 1-3 groups selected from F, —OH, —OCH₃, —N(CH₃)₂, cyclopropyl, morpholinyl, oxadiazolyl, oxetanyl, oxazolyl, pyridinyl, tetrahydrofuranyl, tetrazolyl, thiazolyl and triazolyl, wherein the pyridinyl, thiadiazolyl, thiazolyl and triazolyl are each optionally substituted with F, —CH₃ or —CH₂CH₃; or
R¹ is selected from azabicyclo[3.1.0]hexanyl, azetidinyl, 1H-benzo[d]imidazolyl, bicyclo[1.1.1]pentanyl, cubanyl, cyclobutyl, cyclopropyl, dihydroisoquinolinyl, dihydropyrrolyl, dihydro-2H-benzo[b][1,4]oxazinyl, dioxepanyl, hexahydro-1H-pyrrolizinyl, imidazolidinyl, indazolyl, isoindolinyl, isothiazolyl, morpholinyl, octahydropyrrolo[1,2-a]pyrazinyl, oxabicyclo[3.1.0]hexanyl, 6-oxa-3-azabicyclo[3.1.1]heptanyl, oxabicyclo[3.3]heptanyl, 7-oxa-4-azaspiro[2.5]octanyl, oxetanyl, phenyl, piperidinyl, pyrazolyl, pyridazinyl, pyridinyl, pyrimidinyl, pyrrolidinyl, pyrrolopyridinyl, tetrahydrofuranyl, tetrahydropyranyl and thiazolyl, each of which is optionally substituted with 1 to 3 groups independently selected from R¹¹.

12. The compound of any one of claims 1-11, or a pharmaceutically acceptable salt thereof, wherein R¹ is selected from H, —CH₃, —CH₂(R¹¹), —CH(R¹¹)₂, —CH₂CH₃, —CH(R¹¹)—CH₃, —CH(CH₃)₂, —C(CH₃)₂—R¹⁴, —CH—CH—CH₂—R¹¹, —CH(CH₃)CH₂—R¹¹,

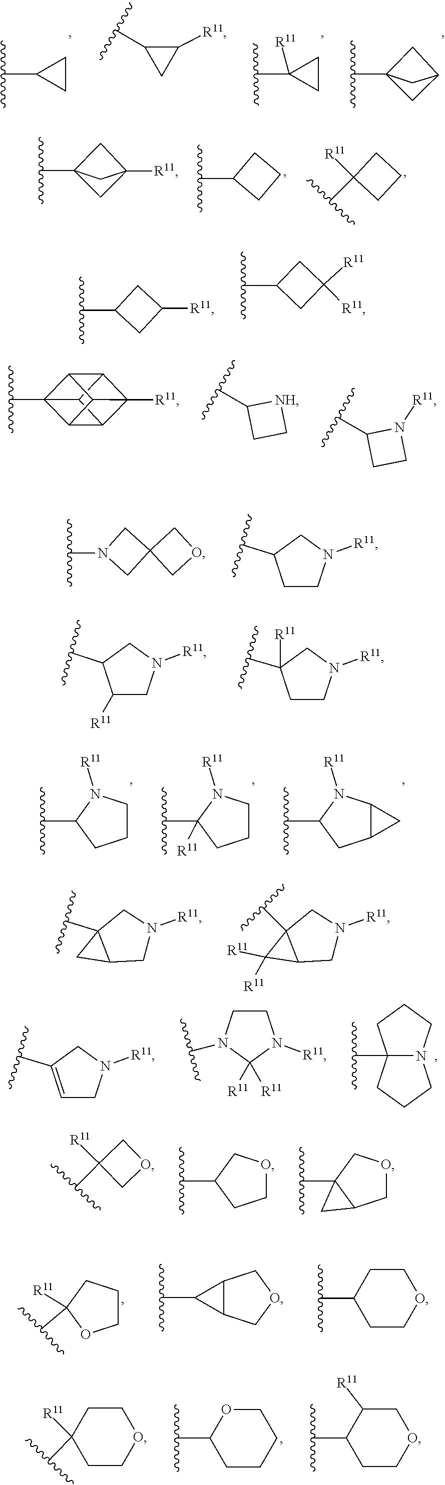

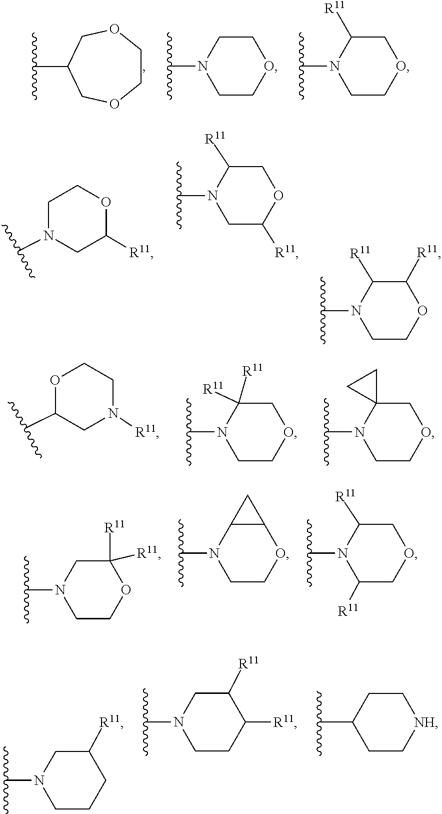

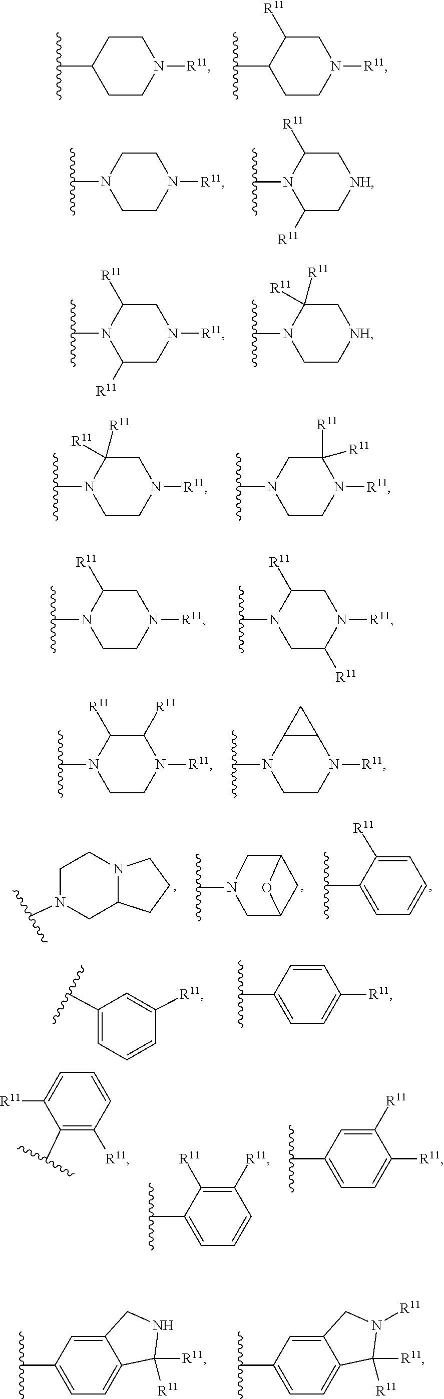

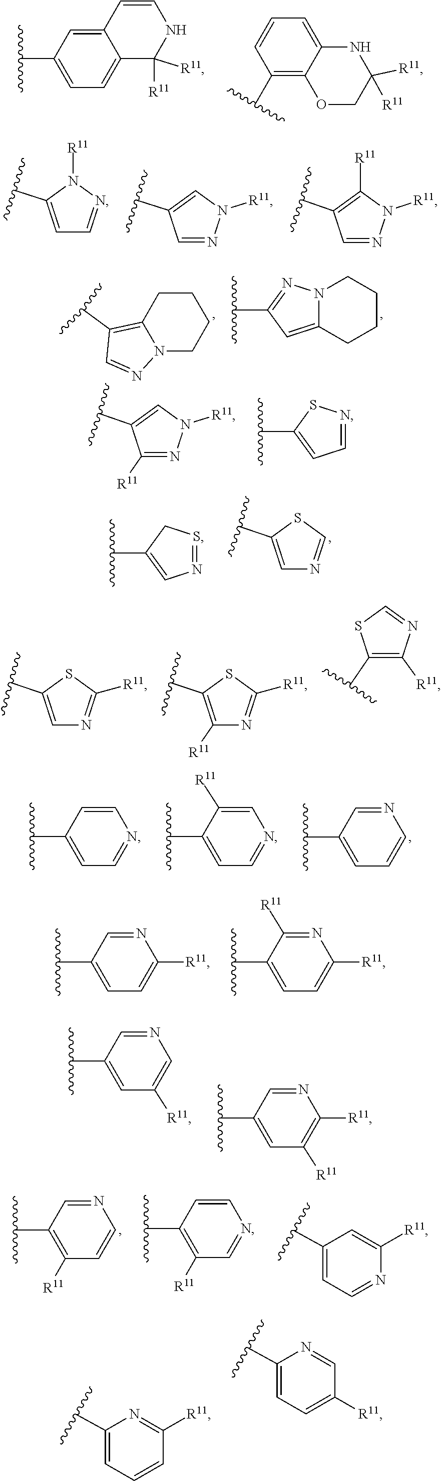

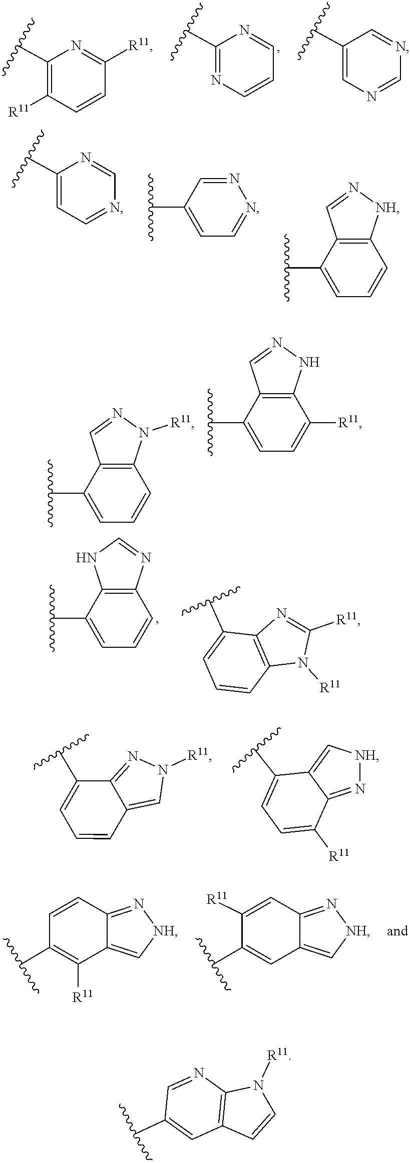

13. The compound of any one of claims 1-12, or a pharmaceutically acceptable salt thereof, wherein:
each R¹¹ is independently selected from halo, OR^{a}, C(O)R^{a}, C(O)NR^{a}R^{b}, NR^{a}C(O)OR^{a}, NR^{a}R^{b}, C₁-C₄alkyl, C₃-C₆cycloalkyl, 4 to 12 membered heterocyclyl, and 5 to 12 membered heteroaryl, wherein the alkyl, cycloalkyl, heterocyclyl, and heteroaryl represented by R¹¹ are each optionally substituted with 1 to 3 groups selected from deuterium, halo, OR^{a} and NR^{a}R^{b}, or two R¹¹ which are attached to the same carbon atom are taken together to form ═O;
each R^{a} is independently selected from H, C₁-C₄alkyl, C₃-C₆cycloalkyl, and 4 to 6 membered heterocyclyl; and
each R^{b} is independently selected from H and C₁-C₄alkyl.

14. The compound of any one of claims 1-13, or a pharmaceutically acceptable salt thereof, wherein:
each R¹¹ is independently selected from halo, OR^{a}, C(O)R^{a}, C(O)NR^{a}R^{b}, NR^{a}C(O)OR^{a}, NR^{a}R^{b}, C₁-C₃alkyl, morpholinyl, oxazolyl, oxadiazolyl, oxetanyl, pyridinyl, tetrahydrofuranyl, tetrazolyl, thiadiazolyl and thiazolyl, wherein the alkyl, morpholinyl, oxazolyl, oxadiazolyl, oxetanyl, pyridinyl, tetrahydrofuranyl, tetrazolyl, thiadiazolyl and thiazolyl represented by R¹¹ are each optionally substituted with 1 to 3 groups selected from deuterium, halo, OR^{a} and NR^{a}R^{b}, or two R¹¹ which are attached to the same carbon atom are taken together to form ═O;
each R^{a} is independently selected from H, —CH₃, —CH₂CH₃, —C(CH₃)₃, 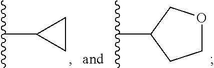
and
each R^{b} is independently H or —CH₃.

15. The compound of any one of claims 1-14, or a pharmaceutically acceptable salt thereof, wherein each R¹¹ is independently selected from Cl, F, —OH, —OCH₃, —C(O)CH₂CH₃, —N(CH₃)₂, —NHC(O)OC(CH₃)₃, —CH₃, —CD₃, —CHF₂, —CF₃, —CH₂OH, —CH₂OCH₃, —CH₂—N(CH₃)₂, —CH₂CH₃, —CH₂CF₃, —CH₂CH₂—N(CH₃)₂, —CH(CH₃)₂, —C(CH₃)₂—OH, 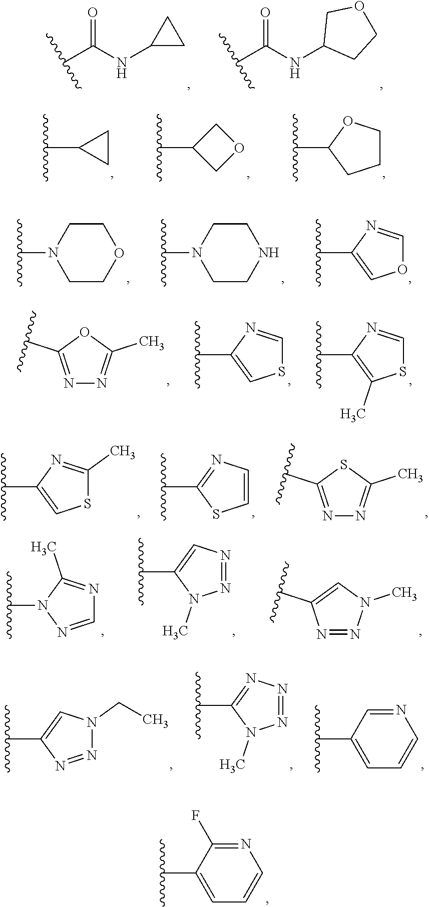
or two R¹¹ which are attached to the same carbon atom are taken together to form ═O.

16. The compound of any one of claims 1-15, or a pharmaceutically acceptable salt thereof, wherein L² is a O.

17. The compound of any one of claims 1-16, or a pharmaceutically acceptable salt thereof, wherein:
R² is C₁-C₄alkyl, C₃-C₈cycloalkyl, C₂-C₄alkenyl or C₂-C₄alkynyl, each of which is optionally substituted with 1 to 3 groups independently selected from halo, OR^{a}, NR^{a}R^{b} and 4 to 12 membered heterocyclyl, or 4 to 12 membered heterocyclyl or 5 to 12 membered heteroaryl, wherein the heterocyclyl and heteroaryl represented by R² are each optionally substituted with 1 to 4 groups selected from C₁-C₄alkyl, C(O)R^{a} and 4 to 12 membered heterocyclyl which is optionally substituted with 1 to 3 groups selected from halo, and C₁-C₄alkyl;
each R^{a} is independently selected from H and C₁-C₄alkyl; and
each R^{b} is independently selected from H and C₁-C₄alkyl.

18. The compound of any one of claims 1-17, or a pharmaceutically acceptable salt thereof, wherein:
R² is C₁-C₃alkyl, C₃-C₈cycloalkyl or C₃-C₄alkynyl, each of which is optionally substituted with 1 to 2 groups independently selected from halo, OR^{a}, NR^{a}R^{b} and 4 to 6 membered heterocyclyl, or 5 to 7 membered heterocyclyl or 5 to 6 membered heteroaryl, wherein the heterocyclyl and heteroaryl in the group represented by R² are each optionally substituted with 1 to 2 groups selected from C₁-C₃alkyl, C(O)R^{a} and 6 membered heterocyclyl optionally substituted with C₁-C₃alkyl;
each R^{a} is independently selected from H and —CH₃; and
each R^{b} is —CH₃.

19. The compound of any one of claims 1-18, or a pharmaceutically acceptable salt thereof, wherein:
R² is —CH₃, —CH₂CH₃, —CH(CH₃)₂, cyclopropyl or 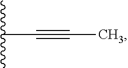 [SMILES: *C#CC]
each of which is optionally substituted with F, —OCH₃, —N(CH₃)₂, morpholinyl or oxetanyl: or
R² is diazaspiro[3.3]heptanyl, pyrazolyl, pyrimidinyl or tetrahydrofuranyl, each of which is optionally substituted with C₁-C₃alkyl, C(O) C₁-C₄alkyl or piperidinyl optionally substituted with —CH₃.

20. The compound of any one of claims 1-19, or a pharmaceutically acceptable salt thereof, wherein:
R² is —CH₃, —CHF₂, —CH₂CH₃, —CH₂CH₂—OCH₃, —CH₂CH₂—N(CH₃)₂, —CH(CH₃)₂, cyclopropyl, 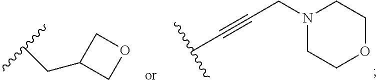 [SMILES: *CC1COC1 ; "or" ; *C#CCN1CCOCC1]
or
R² is 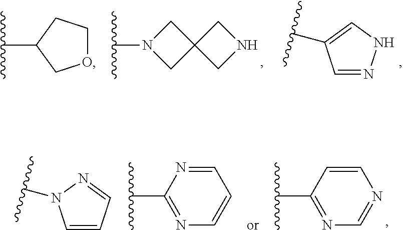
each of which is optionally substituted with —CH₃, C(O)CH₃ or

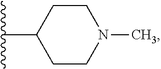 [SMILES: *C1CCN(C)CC1]

21. The compound of any one of claims 1-20, or a pharmaceutically acceptable salt thereof, wherein:
R³ is H, C₁-C₄alkyl, C₃-C₆cycloalkyl or 4 to 6 membered heterocyclyl, wherein the alkyl, cycloalkyl and heterocyclyl represented by R³ are each optionally substituted with 1 to 3 groups independently selected from halo, deuterium, OR^{a}, NR^{a}R^{b} and C₃-C₆cycloalkyl;
each R^{a} is independently H or C₁-C₄alkyl; and
each R^{b} is independently H or C₁-C₄alkyl.

22. The compound of any one of claims 1-21, or a pharmaceutically acceptable salt thereof, wherein R³ is H, cyclopropyl, oxetanyl, tetrahydropyanyl or C₁-C₃alkyl optionally substituted with 1 to 3 groups independently selected from halo, deuterium, OH, N(CH₃)₂ and cyclopropyl.

23. The compound of any one of claims 1-22, or a pharmaceutically acceptable salt thereof, wherein R³ is selected from H, —CH₃, —CHF₂, —CD₃, —CH₂CH₃, —CH₂CHF₂, —CH₂CF₃, —CH₂CH₂—OH, —CH₂CH₂—N(CH₃)₂, —CH₂CH₃CH₃, —CH(CH₃)₂,

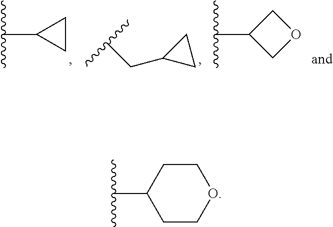

24. The compound of any one of claims 1-23, or a pharmaceutically acceptable salt thereof, wherein:
each R⁴ is halo or OR^{a};
each R^{a} is independently selected from H and C₁-C₄alkyl; and
n is 0, 1, 2 or 3.

25. The compound of any one of claims 1-24, or a pharmaceutically acceptable salt thereof, wherein n is 0.

26. The compound of any one of claims 1-25, or a pharmaceutically acceptable salt thereof, wherein n is 1 or 2, and each R⁴ is independently selected from F and OH.

27. The compound of any one of claim 1-4 or 6-26, wherein the compound is represented by one of the following structural formulas: 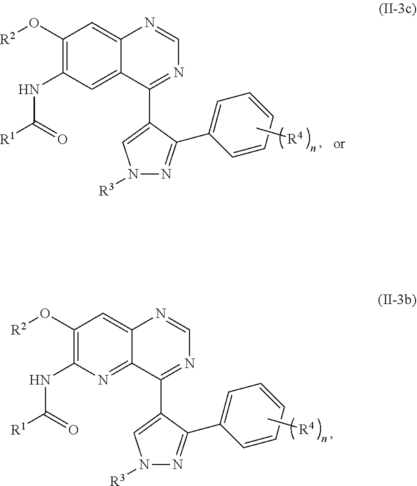
or a pharmaceutically acceptable salt thereof.

28. The compound of any one of claim 1-4 or 6-26, wherein the compound is represented by one of the following structural formulas: 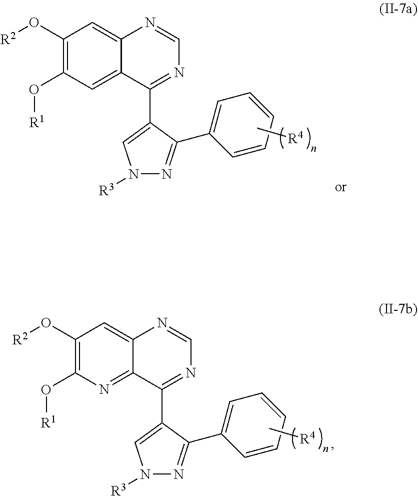
or a pharmaceutically acceptable salt thereof.

29. The compound of claim 27 or 28, wherein:
R¹ is:
C₁-C₄alkyl optionally substituted with 5 to 10 membered heteroaryl, wherein the 5 to 10 membered heteroaryl is optionally substituted with C₁-C₃alkyl, or
C₃-C₈cycloalkyl, 4 to 12 membered heterocyclyl or 5 to 12 membered heteroaryl, wherein the cycloalkyl, heterocyclyl and heteroaryl represented by R¹ are each optionally substituted with 1 to 3 groups independently selected from R¹¹;
each R¹¹ is independently selected from halo, NR^{a}R^{b}, C₃-C₆cycloalkyl, and C₁-C₄alkyl optionally substituted with 1 to 3 halo, and each R^{a} is independently selected from H and C₁-C₄alkyl;
R² and R³ are each independently C₁-C₄alkyl; and
R⁴ is halo, where n is 0, 1, or 2.

30. The compound of any one of claims 27-29, or a pharmaceutically acceptable salt thereof, wherein:
R¹ is:
C₁-C₃alkyl optionally substituted with 5 membered heteroaryl wherein the 5 membered heteroaryl is optionally substituted with C₁-C₃alkyl, or
C₃-C₈cycloalkyl, 5-7 membered heterocyclyl or 5-6 membered heteroaryl, wherein the cycloalkyl, heterocyclyl and heteroaryl represented by R¹ are each optionally substituted with 1 to 2 groups independently selected from R¹¹;
each R¹¹ is independently selected from halo, N(CH₃)₂, C₃-C₈cycloalkyl, and C₁-C₃alkyl optionally substituted with 1 to 3 halo;
R² and R³ are each independently C₁-C₃alkyl;
R⁴ is halo; and
n is 0, 1, or 2.

31. The compound of any one of claims 27-30, or a pharmaceutically acceptable salt thereof, wherein:
R¹ is —CH₂CH₃ substituted with oxadiazolyl optionally substituted with —CH₃; or
R¹ is selected from azabicyclo[3.1.0]hexanyl, bicyclo[1.1.1]pentanyl, cyclopropyl, 3-oxabicyclo[3.1.0]hexanyl, piperidinyl, pyrazolyl and pyridinyl, each of which is optionally substituted with 1 to 2 groups independently selected from R¹¹,
each R¹¹ is independently selected from F, —N(CH₃)₂, —CH₃, —CF₃,

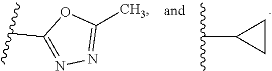

32. The compound of any one of claims 27-31, or a pharmaceutically acceptable salt thereof, wherein R¹ is selected from 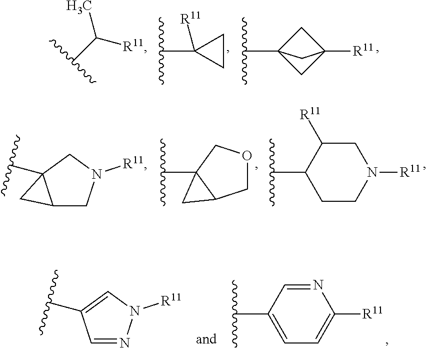
and each R¹¹ is independently selected from F, —N(CH₃)₂, —CH₃, —CF₃,

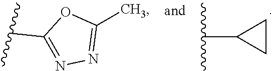

33. The compound of any one of claims 27-32, or a pharmaceutically acceptable salt thereof, wherein:
R² is C₁-C₄alkyl;
R³ is C₁-C₄alkyl;
each R⁴ is independently halo; and
n is 0, 1 or 2.

34. The compound of any one of claims 27-33, or a pharmaceutically acceptable salt thereof, wherein:
R² is —CH₃ or —CH₂CH₃;
R³ is —CH₃;
R⁴ is F; and
n is 0 or 1.

35. The compound of any one of claims 6, 9, and 16-26, wherein the compound is represented by Formula (III): 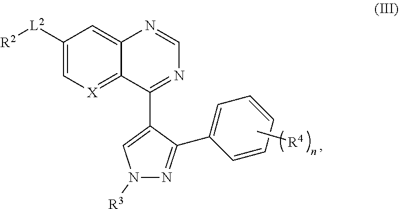
or a pharmaceutically acceptable salt thereof.

36. The compound of any one of claims 6 and 16-26, wherein the compound is represented by Formula (III-1) or (III-2): 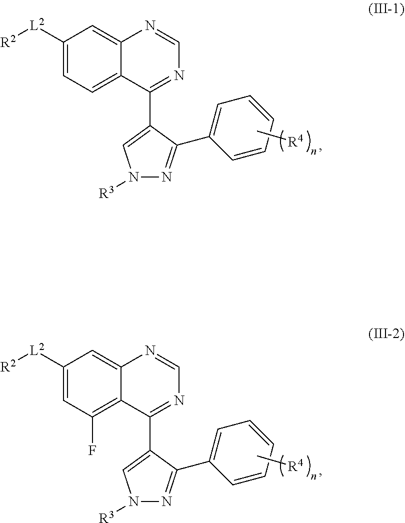
or a pharmaceutically acceptable salt thereof.

37. The compound of claim 35 or 36, or a pharmaceutically acceptable salt thereof, wherein L² is a bond.

38. A pharmaceutical composition comprising a pharmaceutically acceptable carrier and a compound of any one of claims 1-37, or a pharmaceutically acceptable salt thereof.

39. A method of treating a cancer, comprising administering a subject in need thereof an effective amount of a compound of any one of claims 1-37, or a pharmaceutically acceptable salt thereof, or a pharmaceutical composition of claim 38.

40. The method of claim 39, wherein the cancer is lung cancer, colon cancer, urothelial cancer, breast cancer, prostate cancer, brain cancers (glioma), ovarian cancer, gastric cancer, pancreatic cancer, head and neck cancer, bladder cancer, or mesothelioma.

41. The method of claim 39, wherein the cancer is non-small cell lung cancer.

42. The method of any one of claims 39-41, wherein the cancer in the subject in need thereof has metastasized.

43. The method of any one of claims 39-42, wherein the cancer is characterized by an epidermal growth factor receptor (EGFR) L858R mutation.

44. The method of any one of claims 39-42, wherein the cancer is characterized by an epidermal growth factor receptor (EGFR) exon 19 deletion.

45. The method of any one of claims 39-44, wherein the cancer is further characterized by an epidermal growth factor receptor (EGFR) C797S mutation.

46. The method of any one of claims 39-45, wherein the cancer is further characterized by an epidermal growth factor receptor (EGFR) T790M mutation.

47. The method of any one of claims 39-46, further comprises administering the subject in need thereof an effective amount of afatinib or osimertinib.

48. A method of inhibiting epidermal growth factor receptor (EGFR), comprising administering to a subject in need thereof an effective amount of a compound of any of claims 1-37, or a pharmaceutically acceptable salt thereof, or a pharmaceutical composition of claim 38.
